(19) United States
(12) Patent Application Publication
(10) Pub. No.: US 2025/0143305 A1
(43) Pub. Date: May 8, 2025

(54) PYRAZOLE COMPOUND AND PEST CONTROL AGENT CONTAINING THE SAME AS ACTIVE INGREDIENT

(71) Applicant: MITSUI CHEMICALS CROP & LIFE SOLUTIONS, INC., Chuo-ku, Tokyo (JP)

(72) Inventors: Hideaki IKISHIMA, Chiba-shi, Chiba (JP); Shun OKAYA, Chiba-shi, Chiba (JP); Yuuki NODA, Chiba-shi, Chiba (JP); Masaki IMAI, Mobara-shi, Chiba (JP); Takaya YASUDOMI, Mobara-shi, Chiba (JP); Hirotoshi KINJO, Moriyama-shi, Shiga (JP); Hiroki ISHII, Chiba-shi, Chiba (JP); Yukai FUKUTANI, Chiba-shi, Chiba (JP)

(73) Assignee: MITSUI CHEMICALS CROP & LIFE SOLUTIONS, INC., Chuo-ku, Tokyo (JP)

(21) Appl. No.: 18/835,946

(22) PCT Filed: Mar. 10, 2023
(86) PCT No.: PCT/JP2023/009209

§ 371 Date: Aug. 5, 2024

(30) Foreign Application Priority Data
Mar. 11, 2022 (JP) 2022-038675

(51) Int. Cl. A01N 47/18; A01N 43/90; A01P 7/04; C07D 487/04

(52) U.S. Cl. CPC A01N 47/18; A01N 43/90; A01P 7/04; C07D 487/04

(57) ABSTRACT

An object of the present invention is to provide a novel compound for controlling pests. A pyrazole amide compound of the present invention is a novel compound, and can control pests. The pyrazole amide compound of the present invention includes a compound represented by the following Formula (1-1) or Formula (1-2) and a salt thereof.

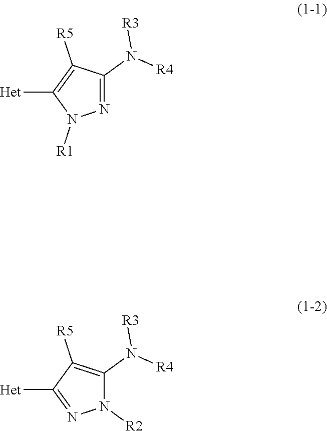

### TECHNICAL FIELD

The present invention relates to a pyrazole compound and a salt thereof, and an arthropod pest control agent containing the compound as an active ingredient.

### BACKGROUND ART

Controlling pests plays an extremely important role in stably and continuously supplying agricultural and horticultural crops. So far, a wide variety of pest control agents have been developed and put into practical use.

However, in recent years, there have been many pests that have acquired drug resistance by using such an existing drug for many years. Therefore, damage in crop production such as agriculture and horticulture is still a serious problem, and it is desired to develop a novel agent that exhibits not only on sensitivity but also effects on resistant pests.

A number of pyrazole compounds having a substituent at the 3-position or 5-position of a pyrazole ring are known. In particular, focusing on an azolopyrimidyl pyrazole compound such as an imidazo[1,2-c]pyrimidine ring and a [1,2,4]triazolo[1,5-c]pyrimidine ring, as a compound having adenosine A2A receptor antagonistic activity, an azolopyrimidyl pyrazole compound having no substituent (see, for example, Patent Literature 1) or an alkyl substituent (see, for example, Patent Literature 2) at the 5-position of a pyrazole ring is disclosed.

### CITATION LIST

### Patent Literatures

Patent Literature 1: CN111018858 (2020)
Patent Literature 2: US20070066620

### SUMMARY OF INVENTION

### Technical Problem

In the azolopyrimidyl pyrazole compound described in the specification of Patent Literature 2, the substituent at the pyrazole 5-position is limited to an alkyl group such as a methyl group, and an azolopyrimidyl pyrazole compound having a substituted amino group or a substituted amide group is not disclosed at all. In addition, the use of the compound group relates to the pharmaceutical field, and is different from the technical field belonging to a pest control agent or an arthropod pest control agent according to the present invention.

An object of the present invention is to provide a compound having an excellent control effect on pests, particularly arthropod pests, mainly in the agricultural field, and an intermediate that is useful for producing the same.

### Solution to Problem

In order to solve the above problems, the present inventors have focused on an azolopyrimidyl pyrazole compound, and as a result of intensive studies, the present inventors have found that a novel azolopyrimidyl pyrazole compound group in which a substituted amino group, a substituted amide group, or the like is introduced at the 3-position or 5-position of the pyrazole ring exhibits an excellent control effect on arthropod pests, thereby completing the present invention.

That is, the present invention includes the following aspects.

A compound represented by Formula (1-1) or Formula (1-2):

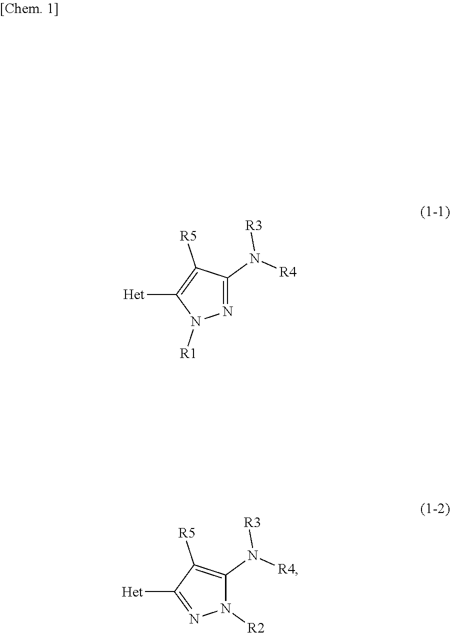

or a salt thereof
[wherein the formula, R1 represents
a hydrogen atom,
a C1 to C6 alkyl group optionally substituted with a substituent A,
a C1 to C6 haloalkyl group,
a C3 to C8 cycloalkyl group optionally substituted with a substituent B,
a C2 to C6 alkenyl group optionally substituted with a substituent A,
a C2 to C6 haloalkenyl group,
a C2 to C6 alkynyl group optionally substituted with a substituent A,
a C2 to C6 haloalkynyl group,
a phenyl group optionally substituted with 0 to 5 substituents B,
a 5- or 6-membered aromatic heterocyclic group optionally substituted with 0 to 4 substituents B,
Rx1Rx2NC(═O)— (where Rx1 and Rx2 each independently represent a hydrogen atom, a hydroxyl group, a cyano group, a C1 to C6 alkyl group optionally substituted with a substituent A, a C1 to C6 haloalkyl group, a C3 to C8 cycloalkyl group optionally substituted with a substituent B, a C2 to C6 alkenyl group optionally substituted with a substituent A, a C2 to C6 haloalkenyl group, a C2 to C6 alkynyl group optionally substituted with a substituent A, a C2 to C6 haloalkynyl group, a C1 to C6 alkoxy group, a C1 to C6 haloalkoxy group, a C3 to C8 cycloalkoxy group, a phenyl group optionally substituted with 0 to 5 substituents B, or a 5- or 6-membered aromatic heterocyclic group optionally substituted with 0 to 4 substituents B, or Rx1 and Rx2 together with a nitrogen atom to which they are bonded form an aziridinyl group, an azetidinyl group, a pyrrolidinyl group, a piperidinyl group, a piperazinyl group, a morpholinyl group, a thiomorpholinyl group, a dioxothiomorpholinyl group, a thiazolidinyl group, an azepanyl group, or an azocanyl group),
Rx3C(═O)— (where Rx3 represents a hydrogen atom, a hydroxyl group, a halogen atom, a C1 to C6 alkyl group optionally substituted with a substituent A, a C1 to C6 haloalkyl group, a C3 to C8 cycloalkyl group optionally substituted with a substituent B, a C2 to C6 alkenyl group optionally substituted with a substituent A, a C2 to C6 haloalkenyl group, a C2 to C6 alkynyl group optionally substituted with a substituent A, a C2 to C6 haloalkynyl group, a phenyl group optionally substituted with 0 to 5 substituents B, or a 5- or 6-membered aromatic heterocyclic group optionally substituted with 0 to 4 substituents B),
Rx3OC(═O)— (where Rx3 has the same meaning as above),
Rx4S(O)p- (where Rx4 represents a hydroxyl group, a halogen atom, a C1 to C6 alkyl group optionally substituted with a substituent A, a C1 to C6 haloalkyl group, a C3 to C8 cycloalkyl group optionally substituted with a substituent B, a C2 to C6 alkenyl group optionally substituted with a substituent A, a C2 to C6 haloalkenyl group, a C2 to C6 alkynyl group optionally substituted with a substituent A, a C2 to C6 haloalkynyl group, or a phenyl group optionally substituted with 0 to 5 substituents B, and p represents an integer of 0, 1, or 2), or
a 3- to 6-membered ring group containing one or two oxygen atoms;
R2 represents
a hydrogen atom,
a C1 to C6 alkyl group optionally substituted with a substituent A,
a C1 to C6 haloalkyl group,
a C3 to C8 cycloalkyl group optionally substituted with a substituent B,
a C2 to C6 alkenyl group optionally substituted with a substituent A,
a C2 to C6 haloalkenyl group,
a C2 to C6 alkynyl group optionally substituted with a substituent A,
a C2 to C6 haloalkynyl group,
a phenyl group optionally substituted with 0 to 5 substituents B,
a 5- or 6-membered aromatic heterocyclic group optionally substituted with 0 to 4 substituents B,
Rx1Rx2NC(═O)— (where, Rx1 and Rx2 have the same meanings as above),
Rx3C(═O)— (where, Rx3 has the same meaning as above),
Rx3OC(═O)— (where, Rx3 has the same meaning as above),
Rx4S(O)p- (where, Rx4 and p have the same meanings as above), or
a 3- to 6-membered ring group containing one or two oxygen atoms;
R3 represents
a hydrogen atom,
a C1 to C6 alkyl group optionally substituted with a substituent A,
a C1 to C6 haloalkyl group,
a C3 to C8 cycloalkyl group optionally substituted with a substituent B,
a C2 to C6 alkenyl group optionally substituted with a substituent A,
a C2 to C6 haloalkenyl group,
a C2 to C6 alkynyl group optionally substituted with a substituent A,
a C2 to C6 haloalkynyl group,
Rx1Rx2NC(═O)— (where, Rx1 and Rx2 have the same meanings as above),
Rx3C(═O)— (where, Rx3 has the same meaning as above),
Rx3OC(═O)— (where, Rx3 has the same meaning as above), or
Rx4S(O)p- (where, Rx4 and p have the same meanings as above);
R4 represents
a hydrogen atom,
a C1 to C6 alkyl group optionally substituted with a substituent A,
a C1 to C6 haloalkyl group,
a C3 to C8 cycloalkyl group optionally substituted with a substituent B,
a C2 to C6 alkenyl group optionally substituted with a substituent A,
a C2 to C6 haloalkenyl group,
a C2 to C6 alkynyl group optionally substituted with a substituent A,
a C2 to C6 haloalkynyl group,
Rx1Rx2NC(═O)— (where, Rx1 and Rx2 have the same meanings as above),
Rx3C(═O)— (where, Rx3 has the same meaning as above),
Rx3OC(═O)— (where, Rx3 has the same meaning as above), or
Rx4S(O)p- (where, Rx4 and p have the same meanings as above);
R5 represents
a cyano group,
a halogen atom,
a C1 to C6 alkoxy group optionally substituted with a substituent A,
a C1 to C6 haloalkoxy group,
a C3 to C8 cycloalkoxy group optionally substituted with a substituent A,
a C2 to C6 alkenyl group optionally substituted with a substituent A,
a C2 to C6 haloalkenyl group,
a C2 to C6 alkynyl group optionally substituted with a substituent A,
a C2 to C6 haloalkynyl group, or
Rx4S(O)p- (where, Rx4 and p have the same meanings as above);
Het represents Formula (Het);

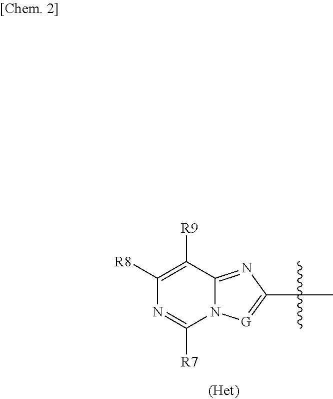

G represents C—R6 or a nitrogen atom;
R6 represents
a hydrogen atom,
a cyano group,
a halogen atom,
a C1 to C6 alkyl group optionally substituted with a substituent A,
a C1 to C6 haloalkyl group,
a C3 to C8 cycloalkyl group optionally substituted with a substituent B,
a C2 to C6 alkenyl group optionally substituted with a substituent A,
a C2 to C6 haloalkenyl group,
a C2 to C6 alkynyl group optionally substituted with a substituent A, or
a C2 to C6 haloalkynyl group;
R7 represents
a hydrogen atom,
a hydroxyl group,
a cyano group,
a halogen atom,
a C1 to C6 alkyl group optionally substituted with a substituent A,
a C1 to C6 haloalkyl group,
a C3 to C8 cycloalkyl group optionally substituted with a substituent B,
a C2 to C6 alkenyl group optionally substituted with a substituent A,
a C2 to C6 haloalkenyl group,
a C2 to C6 alkynyl group optionally substituted with a substituent A,
a C2 to C6 haloalkynyl group,
a C1 to C6 alkoxy group optionally substituted with a substituent A,
a C1 to C6 haloalkoxy group,
a C3 to C8 cycloalkoxy group optionally substituted with a substituent A, or
Rx4S(O)p- (where, Rx4 and p have the same meanings as above);
R8 represents
a hydrogen atom,
a hydroxyl group,
a cyano group,
a halogen atom,
a C1 to C6 alkyl group optionally substituted with a substituent A,
a C1 to C6 haloalkyl group,
a C3 to C8 cycloalkyl group optionally substituted with a substituent B,
a C1 to C6 alkoxy group optionally substituted with a substituent A,
a C1 to C6 haloalkoxy group,
a C3 to C8 cycloalkoxy group optionally substituted with a substituent A, or
Rx4S(O)p- (where, Rx4 and p have the same meanings as above);
R9 represents
a hydrogen atom,
a hydroxyl group,
a cyano group,
a halogen atom,
a C1 to C6 alkyl group optionally substituted with a substituent A,
a C1 to C6 haloalkyl group,
a C3 to C8 cycloalkyl group optionally substituted with a substituent B,
a C1 to C6 alkoxy group optionally substituted with a substituent A,
a C1 to C6 haloalkoxy group,
a C3 to C8 cycloalkoxy group optionally substituted with a substituent A, or
Rx4S(O)p- (where, Rx4 and p have the same meanings as above);
the substituent A is at least one selected from the group consisting of
a hydroxyl group,
a cyano group,
a nitro group,
a halogen atom,
a C3 to C8 cycloalkyl group,
a C1 to C6 alkoxy group,
a C1 to C6 haloalkoxy group,
a C3 to C8 cycloalkoxy group,
a phenyl group optionally substituted with 0 to 5 substituents C,
a 5- or 6-membered aromatic heterocyclic group optionally substituted with 0 to 4 substituents C, and
a 3- to 6-membered ring group containing one or two oxygen atoms;
the substituent B is at least one selected from the group consisting of
a hydroxyl group,
a cyano group,
a nitro group,
a halogen atom,
a C1 to C6 alkyl group,
a C1 to C6 haloalkyl group,
a C3 to C8 cycloalkyl group,
a C1 to C6 alkoxy group,
a C1 to C6 haloalkoxy group,
a C3 to C8 cycloalkoxy group, and
Rx5S(O)p- (where Rx5 represents a hydroxyl group, a halogen atom, a C1 to C6 alkyl group, a C1 to C6 haloalkyl group, a C3 to C8 cycloalkyl group, a C2 to C6 alkenyl group, a C2 to C6 haloalkenyl group, a C2 to C6 alkynyl group, or a C2 to C6 haloalkynyl group, and p has the same meaning as above); and
the substituent C is at least one independently selected from the group consisting of
a cyano group,
a halogen atom,
a C1 to C6 alkyl group,
a C1 to C6 haloalkyl group,
a C3 to C8 cycloalkyl group,
a C1 to C6 alkoxy group,
a C3 to C6 haloalkoxy group,
a C3 to C8 cycloalkoxy group, and
Rx5S(O)p- (where, Rx5 and p have the same meanings as above)].
   [2]

The compound or the salt thereof described in [1], wherein R1 represents
a hydrogen atom,
a C1 to C6 alkyl group optionally substituted with a substituent A,
a C1 to C6 haloalkyl group,
a C3 to C8 cycloalkyl group optionally substituted with a substituent B,
a C2 to C6 alkenyl group optionally substituted with a substituent A,
a C2 to C6 alkynyl group optionally substituted with a substituent A, or
Rx1Rx2NC(═O)— (where Rx1 and Rx2 each independently represent a hydrogen atom, a hydroxyl group, a cyano group, a C1 to C6 alkyl group optionally substituted with a substituent A, a C1 to C6 haloalkyl group, a C3 to C8 cycloalkyl group optionally substituted with a substituent B, a C2 to C6 alkenyl group optionally substituted with a substituent A, a C2 to C6 haloalkenyl group, a C2 to C6 alkynyl group optionally substituted with a substituent A, a C2 to C6 haloalkynyl group, a C1 to C6 alkoxy group, a C1 to C6 haloalkoxy group, a C3 to C8 cycloalkoxy group, a phenyl group optionally substituted with 0 to 5 substituents B, or a 5- or 6-membered aromatic heterocyclic group optionally substituted with 0 to 4 substituents B, or Rx1 and Rx2 together with a nitrogen atom to which they are bonded form an aziridinyl group, an azetidinyl group, a pyrrolidinyl group, a piperidinyl group, a piperazinyl group, a morpholinyl group, a thiomorpholinyl group, a dioxothiomorpholinyl group, a thiazolidinyl group, an azepanyl group, or an azocanyl group);
R2 represents
a hydrogen atom,
a C1 to C6 alkyl group optionally substituted with a substituent A,
a C1 to C6 haloalkyl group,
a C3 to C8 cycloalkyl group optionally substituted with a substituent B,
a C2 to C6 alkenyl group optionally substituted with a substituent A,
a C2 to C6 alkynyl group optionally substituted with a substituent A, or
Rx1Rx2NC(═O)— (where Rx1 and Rx2 have the same meanings as above);
R3 represents
a hydrogen atom,
a C1 to C6 alkyl group optionally substituted with a substituent A,
a C1 to C6 haloalkyl group,
a C3 to C6 cycloalkyl group optionally substituted with a substituent B,
a C2 to C6 alkenyl group optionally substituted with a substituent A,
a C2 to C6 alkynyl group optionally substituted with a substituent A,
Rx1Rx2NC(═O)— (where Rx1 and Rx2 have the same meanings as above),
Rx3C(═O)— (where Rx3 represents a hydrogen atom, a hydroxyl group, a halogen atom, a C1 to C6 alkyl group optionally substituted with a substituent A, a C1 to C6 haloalkyl group, a C3 to C8 cycloalkyl group optionally substituted with a substituent B, a C2 to C6 alkenyl group optionally substituted with a substituent A, a C2 to C6 haloalkenyl group, a C2 to C6 alkynyl group optionally substituted with a substituent A, a C2 to C6 haloalkynyl group, a phenyl group optionally substituted with 0 to 5 substituents B, or a 5- or 6-membered aromatic heterocyclic group optionally substituted with 0 to 4 substituents B), or
Rx3OC(═O)— (where Rx3 has the same meaning as above);
R4 represents
a hydrogen atom,
a C1 to C6 alkyl group optionally substituted with a substituent A,
a C1 to C6 haloalkyl group,
a C3 to C8 cycloalkyl group optionally substituted with a substituent B,
a C2 to C6 alkenyl group optionally substituted with a substituent A,
a C2 to C6 alkynyl group optionally substituted with a substituent A,
Rx1Rx2NC(═O)— (where Rx1 and Rx2 have the same meanings as above),
Rx3C(═O)— (where Rx3 has the same meaning as above),
Rx3OC(═O)— (where Rx3 has the same meaning as above), or
Rx4S(O)p- (where Rx4 represents a hydroxyl group, a halogen atom, a C1 to C6 alkyl group optionally substituted with a substituent A, a C1 to C6 haloalkyl group, a C3 to C8 cycloalkyl group optionally substituted with a substituent B, a C2 to C6 alkenyl group optionally substituted with a substituent A, a C2 to C6 haloalkenyl group, a C2 to C6 alkynyl group optionally substituted with a substituent A, a C2 to C6 haloalkynyl group, or a phenyl group optionally substituted with 0 to 5 substituents B, and p represents an integer of 0, 1, or 2);
R5 represents
a cyano group,
a halogen atom,
a C1 to C6 alkoxy group optionally substituted with a substituent A,
a C1 to C6 haloalkoxy group,
a C3 to C8 cycloalkoxy group optionally substituted with a substituent A, or
Rx4S(O)p- (where Rx4 and p have the same meanings as above);
R6 represents
a hydrogen atom,
a cyano group,
a halogen atom,
a C1 to C6 alkyl group optionally substituted with a substituent A,
a C1 to C6 haloalkyl group, or
a C3 to C8 cycloalkyl group optionally substituted with a substituent B;
R7 represents
a hydrogen atom,
a hydroxyl group,
a cyano group,
a halogen atom,
a C1 to C6 alkyl group optionally substituted with a substituent A,
a C1 to C6 haloalkyl group,
a C3 to C8 cycloalkyl group optionally substituted with a substituent B,
a C1 to C6 alkoxy group optionally substituted with a substituent A,
a C1 to C6 haloalkoxy group,
a C3 to C8 cycloalkoxy group optionally substituted with a substituent A, or
Rx4S(O)p- (where Rx4 and p have the same meanings as above);
R8 represents
a hydrogen atom,
a halogen atom,
a C1 to C6 alkyl group optionally substituted with a substituent A,
a C1 to C6 haloalkyl group,
a C3 to C8 cycloalkyl group optionally substituted with a substituent B,
a C1 to C6 haloalkoxy group, or
Rx4S(O)p- (where Rx4 and p have the same meanings as above); and
R9 represents
a hydrogen atom,
a halogen atom,
a C1 to C6 alkyl group optionally substituted with a substituent A,
a C1 to C6 haloalkyl group, or
Rx4S(O)p- (where Rx4 and p have the same meanings as above).
   [3]

The compound or the salt thereof described in [2], wherein R1 represents
a hydrogen atom,
a C1 to C6 alkyl group optionally substituted with a substituent A, or
a C1 to C6 haloalkyl group;
R2 represents
a hydrogen atom,
a C1 to C6 alkyl group optionally substituted with a substituent A,
a C1 to C6 haloalkyl group,
a C3 to C8 cycloalkyl group optionally substituted with a substituent B, or
Rx1Rx2NC(═O)— (where Rx1 and Rx2 each independently represent a hydrogen atom, a hydroxyl group, a cyano group, a C1 to C6 alkyl group optionally substituted with a substituent A, a C1 to C6 haloalkyl group, a C3 to C8 cycloalkyl group optionally substituted with a substituent B, a C2 to C6 alkenyl group optionally substituted with a substituent A, a C2 to C6 haloalkenyl group, a C2 to C6 alkynyl group optionally substituted with a substituent A, a C2 to C6 haloalkynyl group, a C1 to C6 alkoxy group, a C1 to C6 haloalkoxy group, a C3 to C8 cycloalkoxy group, a phenyl group optionally substituted with 0 to 5 substituents B, or a 5- or 6-membered aromatic heterocyclic group optionally substituted with 0 to 4 substituents B, or Rx1 and Rx2 together with a nitrogen atom to which they are bonded form an aziridinyl group, an azetidinyl group, a pyrrolidinyl group, a piperidinyl group, a piperazinyl group, a morpholinyl group, a thiomorpholinyl group, a dioxothiomorpholinyl group, a thiazolidinyl group, an azepanyl group, or an azocanyl group);
R3 represents
a hydrogen atom,
a C1 to C6 alkyl group optionally substituted with a substituent A,
a C1 to C6 haloalkyl group,
a C3 to C8 cycloalkyl group optionally substituted with a substituent B,
Rx1Rx2NC(═O)— (where Rx1 and Rx2 have the same meanings as above),
Rx3C(═O)— (where Rx3 represents a hydrogen atom, a hydroxyl group, a halogen atom, a C1 to C6 alkyl group optionally substituted with a substituent A, a C1 to C6 haloalkyl group, a C3 to C8 cycloalkyl group optionally substituted with a substituent B, a C2 to C6 alkenyl group optionally substituted with a substituent A, a C2 to C6 haloalkenyl group, a C2 to C6 alkynyl group optionally substituted with a substituent A, a C2 to C6 haloalkynyl group, a phenyl group optionally substituted with 0 to 5 substituents B, or a 5- or 6-membered aromatic heterocyclic group optionally substituted with 0 to 4 substituents B), or
Rx3OC(═O)— (where Rx3 has the same meaning as above);
R4 represents
a hydrogen atom,
a C1 to C6 alkyl group optionally substituted with a substituent A,
a C1 to C6 haloalkyl group,
a C3 to C8 cycloalkyl group optionally substituted with a substituent B,
Rx1Rx2NC(═O)— (where Rx1 and Rx2 have the same meanings as above),
Rx3C(═O)— (where Rx3 has the same meaning as above),
Rx3OC(═O)— (where Rx3 has the same meaning as above), or
Rx4S(O)p-(where Rx4 represents a hydroxyl group, a halogen atom, a C1 to C6 alkyl group optionally substituted with a substituent A, a C1 to C6 haloalkyl group, a C3 to C8 cycloalkyl group optionally substituted with a substituent B, a C2 to C6 alkenyl group optionally substituted with a substituent A, a C2 to C6 haloalkenyl group, a C2 to C6 alkynyl group optionally substituted with a substituent A, a C2 to C6 haloalkynyl group, or a phenyl group optionally substituted with 0 to 5 substituents B, and p represents an integer of 0, 1, or 2);
R5 represents
a cyano group,
a halogen atom,
a C1 to C6 alkoxy group optionally substituted with a substituent A, or
Rx4S(O)p- (where Rx4 and p have the same meanings as above);
R6 represents
a hydrogen atom,
a halogen atom,
a C1 to C6 alkyl group optionally substituted with a substituent A, or
a C1 to C6 haloalkyl group;
R7 represents
a hydrogen atom,
a hydroxyl group,
a cyano group,
a halogen atom,
a C1 to C6 alkyl group optionally substituted with a substituent A,
a C1 to C6 haloalkyl group,
a C3 to C8 cycloalkyl group optionally substituted with a substituent B,
a C1 to C6 alkoxy group optionally substituted with a substituent A,
a C1 to C6 haloalkoxy group, or
Rx4S(O)p- (where Rx4 and p have the same meanings as above);
R8 represents
a halogen atom,
a C1 to C6 haloalkyl group,
a C1 to C6 haloalkoxy group, or
Rx4S(O)p- (where Rx4 and p have the same meanings as above); and
R9 represents
a hydrogen atom,
a halogen atom, or
a C1 to C6 haloalkyl group.
   [4]

The compound or the salt thereof described in [3], wherein R1 represents
a C1 to C6 alkyl group optionally substituted with a substituent A, or
a C1 to C6 haloalkyl group;
R2 represents
a C1 to C6 alkyl group optionally substituted with a substituent A,
a C1 to C6 haloalkyl group, or
a C3 to C8 cycloalkyl group optionally substituted with a substituent B;
R3 represents
a hydrogen atom,
a C1 to C6 alkyl group optionally substituted with a substituent A,
a C1 to C6 haloalkyl group, or
Rx3C(═O)— (where Rx3 represents a hydrogen atom, a hydroxyl group, a halogen atom, a C1 to C6 alkyl group optionally substituted with a substituent A, a C1 to C6 haloalkyl group, a C3 to C8 cycloalkyl group optionally substituted with a substituent B, a C2 to C6 alkenyl group optionally substituted with a substituent A, a C2 to C6 haloalkenyl group, a C2 to C6 alkynyl group optionally substituted with a substituent A, a C2 to C6 haloalkynyl group, a phenyl group optionally substituted with 0 to 5 substituents B, or a 5- or 6-membered aromatic heterocyclic group optionally substituted with 0 to 4 substituents B);
R4 represents
a hydrogen atom,
a C1 to C6 alkyl group optionally substituted with a substituent A,
Rx1Rx2NC(═O)— (where Rx1 and Rx2 each independently represent a hydrogen atom, a hydroxyl group, a cyano group, a C1 to C6 alkyl group optionally substituted with a substituent A, a C1 to C6 haloalkyl group, a C3 to C8 cycloalkyl group optionally substituted with a substituent B, a C2 to C6 alkenyl group optionally substituted with a substituent A, a C2 to C6 haloalkenyl group, a C2 to C6 alkynyl group optionally substituted with a substituent A, a C2 to C6 haloalkynyl group, a C1 to C6 alkoxy group, a C1 to C6 haloalkoxy group, a C3 to C8 cycloalkoxy group, a phenyl group optionally substituted with 0 to 5 substituents B, or a 5- or 6-membered aromatic heterocyclic group optionally substituted with 0 to 4 substituents B, or Rx1 and Rx2 together with a nitrogen atom to which they are bonded form an aziridinyl group, an azetidinyl group, a pyrrolidinyl group, a piperidinyl group, a piperazinyl group, a morpholinyl group, a thiomorpholinyl group, a dioxothiomorpholinyl group, a thiazolidinyl group, an azepanyl group, or an azocanyl group),
Rx3C(═O)— (where Rx3 has the same meaning as above),
Rx3OC(═O)— (where Rx3 has the same meaning as above), or
Rx4S(O)p- (where Rx4 represents a hydroxyl group, a halogen atom, a C1 to C6 alkyl group optionally substituted with a substituent A, a C1 to C6 haloalkyl group, a C3 to C8 cycloalkyl group optionally substituted with a substituent B, a C2 to C6 alkenyl group optionally substituted with a substituent A, a C2 to C6 haloalkenyl group, a C2 to C6 alkynyl group optionally substituted with a substituent A, a C2 to C6 haloalkynyl group, or a phenyl group optionally substituted with 0 to 5 substituents B, and p represents an integer of 0, 1, or 2);
R5 represents
Rx4S(O)p- (where Rx4 and p have the same meanings as above);
R6 represents
a hydrogen atom,
a halogen atom, or
a C1 to C6 alkyl group optionally substituted with a substituent A;
R7 represents
a hydrogen atom,
a hydroxyl group,
a halogen atom,
a C1 to C6 alkyl group optionally substituted with a substituent A,
a C3 to C8 cycloalkyl group optionally substituted with a substituent B,
a C1 to C6 alkoxy group optionally substituted with a substituent A,
a C1 to C6 haloalkoxy group, or
Rx4S(O)p- (where Rx4 and p have the same meanings as above);
R8 represents
a C1 to C6 haloalkyl group; and
R9 represents
a hydrogen atom.
   [5]

The compound or the salt thereof described in any one of [1] to [4], wherein R1 represents a pyrimidin-5-ylmethyl group or a 2,2,2-trifluoroethyl group.

[6]

The compound or the salt thereof described in any one of [1] to [4] and [5], wherein R2 represents a methyl group, an ethyl group, a propyl group, a pyrimidin-5-ylmethyl group, a 2,2,2-trifluoroethyl group, or a cyclopropyl group.

[7]

The compound or the salt thereof described in any one of [1] to [4], [5], and [6], wherein R3 represents a hydrogen atom, a methyl group, a 2,2,2-trifluoroethyl group, an acetyl group, a cyclohexylcarbonyl group, or a 4-trifluoromethoxybenzoyl group.

[8]

The compound or the salt thereof described in any one of [1] to [4] and [5] to [7], wherein R4 represents a hydrogen atom, a methyl group, a methylcarbamoyl group, a dimethylcarbamoyl group, an acetyl group, a propionyl group, an isopropionyl group, a butyryl group, an isobutyryl group, a pentanoyl group, a 3-methyl-1-butyryl group, a 3-ethyl-1-pentanoyl group, a (1H-pyrazol-1-yl)acetyl group, a (1H-1,2,4-triazol-1-yl)acetyl group, a cyclopropylmethylcarbonyl group, a 2-chloroacetyl group, a 2,2-difluoroacetyl group, a 2,2-dichloroacetyl group, a 2,2,2-trifluoroacetyl group, a 2-chloro-2,2-difluoroacetyl group, a 2,2-dichloro-2-fluoroacetyl group, a 2,2,2-trichloroacetyl group, a 3,3-difluoropropionyl group, a 2,2,3,3-tetrafluoropropionyl group, a 2,2,3,3,3-pentafluoropropionyl group, a 2,2-difluoropropionyl group, a 3,3,3-trifluoropropionyl group, a 3-chloro-2,2,3,3-tetrafluoropropionyl group, a 3,3,3-trifluoro-2,2-dimethylpropionyl group, a 4,4,4-trifluorobutyryl group, a 2,2,3,3,4,4,4-heptafluorobutyryl group, a 4,4,4-trifluoro-3-(trifluoromethyl)butyryl group, a 2,2,3,3,4,4,5,5,5-nonafluoropentanoyl group, a 2,2,3,3,4,4,5,5,6,6,6-undecafluorohexanoyl group, a cyclopropylcarbonyl group, a 1-fluorocyclopropyl-1-carbonyl group, a 1-trifluoromethylcyclopropyl-1-carbonyl group, a 1-cyanocyclopropyl-1-carbonyl group, a 1-methylcyclopropyl-1-carbonyl group, a 2,2,3,3-tetramethyl-1-cyclopropylcarbonyl group, a cyclobutylcarbonyl group, a 1-trifluoromethylcyclobutyl-1-carbonyl group, a cyclopentylcarbonyl group, a cyclohexylcarbonyl group, an acryloyl group, a 2-fluoroacryloyl group, a 3-ethoxyacryloyl group, a benzoyl group, a 2-fluorobenzoyl group, a 3-fluorobenzoyl group, a 4-fluorobenzoyl group, a 2-chlorobenzoyl group, a 3-chlorobenzoyl group, a 4-chlorobenzoyl group, a 2-methylbenzoyl group, a 3-methylbenzoyl group, a 4-methylbenzoyl group, a 4-ethylbenzoyl group, a 3,4-dimethylbenzoyl group, a 3,5-dimethylbenzoyl group, a 2-trifluoromethylbenzoyl group, a 3-trifluoromethylbenzoyl group, a 4-trifluoromethylbenzoyl group, a 2-methoxybenzoyl group, a 3-methoxybenzoyl group, a 4-methoxybenzoyl group, a 4-trifluoromethoxybenzoyl group, a picolinoyl group, a 3-chloropicolinoyl group, a 4-chloropicolinoyl group, a 5-chloropicolinoyl group, a 6-chloropicolinoyl group, a 6-methylpicolinoyl group, a nicotinoyl group, a 2-chloronicotinoyl group, a 4-chloronicotinoyl group, a 5-chloronicotinoyl group, a 6-chloronicotinoyl group, a 6-trifluoromethylpicolinoyl group, a 2-chloro isonicotinoyl group, a 3-chloroisonicotinoyl group, a pyrimidine-2-carbonyl group, a pyrimidine-4-carbonyl group, a 2-chloro-pyrimidine-4-carbonyl group, a 2-methyl-pyrimidine-4-carbonyl group, a 2-trifluoromethyl-pyrimidine-4-carbonyl group, a 6-methyl-pyrimidine-4-carbonyl group, a pyrazine-2-carbonyl group, a methoxycarbonyl group, an ethoxycarbonyl group, a propyloxycarbonyl group, a t-butyloxycarbonyl group, a 2,2,2-trifluoroethoxycarbonyl group, or a methylsulfonyl group.

[9]

The compound or the salt thereof described in any one of [1] to [4] and [5] to [8], wherein R5 represents an ethylsulfanyl group, an ethylsulfinyl group, or an ethylsulfonyl group.

[10]

The compound or the salt thereof described in any one of [1] to [4] and [5] to [9], wherein R6 represents a hydrogen atom, a chlorine atom, a bromine atom, or a methyl group.

[11]

The compound or the salt thereof described in any one of [1] to [4] and [5] to [10], wherein R7 represents a hydrogen atom, a hydroxyl group, a chlorine atom, a methyl group, a cyclopropyl group, a methoxy group, an ethoxy group, a propyloxy group, a 2,2,2-trifluoroethoxy group, a methylsulfanyl group, or a methylsulfonyl group.

[12]

The compound or the salt thereof described in any one of [1] to [4] and [5] to [11], wherein R8 represents a difluoromethyl group, a trifluoromethyl group, a dichloromethyl group, a chlorodifluoromethyl group, a pentafluoroethyl group, a 2-fluoroisopropyl group, or a heptafluoropropyl group.

[13]

The compound or the salt thereof described in any one of [1] to [4] and [5] to [12], wherein R9 represents a hydrogen atom.

[14]

A pest control agent containing the compound or the salt thereof described in any one of [1] to [4] and [5] to [13] as an active ingredient.

[15]

The pest control agent described in [14], wherein the pest is an arthropod pest.

[16]

A method for controlling a pest, comprising applying the pest control agent according to any one of [1] to [4] and [5] to [13] to a plant, a seed of a plant, or soil for cultivating a plant.

[17]

The method described in [16], wherein the pest is an arthropod pest.

[18]

A use of the compound or the salt thereof described in any one of [1] to [4] and [5] to [13] as a pest control agent.

[19]

The use described in [18], wherein the pest is an arthropod pest.

[20]

A compound represented by Formula (1-3) or Formula (1-4),

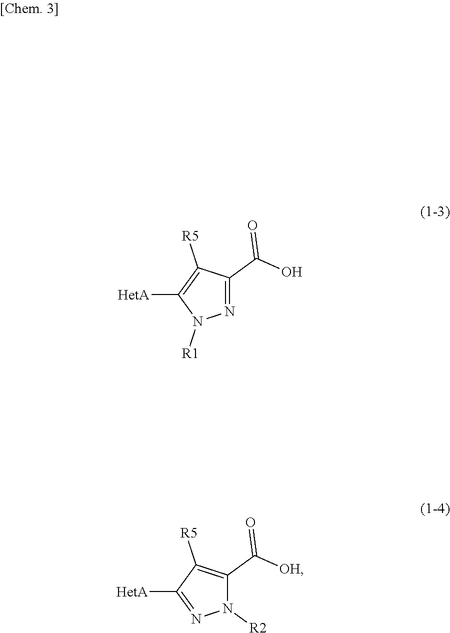

or a salt thereof
[wherein, R1 represents
a hydrogen atom,
a C1 to C6 alkyl group optionally substituted with a substituent A,
a C1 to C6 haloalkyl group,
a C3 to C8 cycloalkyl group optionally substituted with a substituent B,
a C2 to C6 alkenyl group optionally substituted with a substituent A,
a C2 to C6 haloalkenyl group,
a C2 to C6 alkynyl group optionally substituted with a substituent A,
a C2 to C6 haloalkynyl group,
a phenyl group optionally substituted with 0 to 5 substituents B,
a 5- or 6-membered aromatic heterocyclic group optionally substituted with 0 to 4 substituents B,
Rx1Rx2NC(═O)— (where Rx1 and Rx2 each independently represent a hydrogen atom, a hydroxyl group, a cyano group, a C1 to C6 alkyl group optionally substituted with a substituent A, a C1 to C6 haloalkyl group, a C3 to C8 cycloalkyl group optionally substituted with a substituent B, a C2 to C6 alkenyl group optionally substituted with a substituent A, a C2 to C6 haloalkenyl group, a C2 to C6 alkynyl group optionally substituted with a substituent A, a C2 to C6 haloalkynyl group, a C1 to C6 alkoxy group, a C1 to C6 haloalkoxy group, a C3 to C8 cycloalkoxy group, a phenyl group optionally substituted with 0 to 5 substituents B, or a 5- or 6-membered aromatic heterocyclic group optionally substituted with 0 to 4 substituents B, or Rx1 and Rx2 together with a nitrogen atom to which they are bonded form an aziridinyl group, an azetidinyl group, a pyrrolidinyl group, a piperidinyl group, a piperazinyl group, a morpholinyl group, a thiomorpholinyl group, a dioxothiomorpholinyl group, a thiazolidinyl group, an azepanyl group, or an azocanyl group),
Rx3C(═O)— (where Rx3 represents a hydrogen atom, a hydroxyl group, a halogen atom, a C1 to C6 alkyl group optionally substituted with a substituent A, a C1 to C6 haloalkyl group, a C3 to C8 cycloalkyl group optionally substituted with a substituent B, a C2 to C6 alkenyl group optionally substituted with a substituent A, a C2 to C6 haloalkenyl group, a C2 to C6 alkynyl group optionally substituted with a substituent A, a C2 to C6 haloalkynyl group, a phenyl group optionally substituted with 0 to 5 substituents B, or a 5- or 6-membered aromatic heterocyclic group optionally substituted with 0 to 4 substituents B),
Rx3OC(═O)— (where Rx3 has the same meaning as above),
Rx4S(O)p- (where Rx4 represents a hydroxyl group, a halogen atom, a C1 to C6 alkyl group optionally substituted with a substituent A, a C1 to C6 haloalkyl group, a C3 to C8 cycloalkyl group optionally substituted with a substituent B, a C2 to C6 alkenyl group optionally substituted with a substituent A, a C2 to C6 haloalkenyl group, a C2 to C6 alkynyl group optionally substituted with a substituent A, a C2 to C6 haloalkynyl group, or a phenyl group optionally substituted with 0 to 5 substituents B, and p represents an integer of 0, 1, or 2), or
a 3- to 6-membered ring group containing one or two oxygen atoms;
R2 represents
a hydrogen atom,
a C1 to C6 alkyl group optionally substituted with a substituent A,
a C1 to C6 haloalkyl group,
a C3 to C8 cycloalkyl group optionally substituted with a substituent B,
a C2 to C6 alkenyl group optionally substituted with a substituent A,
a C2 to C6 haloalkenyl group,
a C2 to C6 alkynyl group optionally substituted with a substituent A,
a C2 to C6 haloalkynyl group,
a phenyl group optionally substituted with 0 to 5 substituents B,
a 5- or 6-membered aromatic heterocyclic group optionally substituted with 0 to 4 substituents B,
Rx1Rx2NC(═O)— (where, Rx1 and Rx2 have the same meanings as above),
Rx3C(═O)— (where, Rx3 has the same meaning as above),
Rx3OC(═O)— (where, Rx3 has the same meaning as above),
Rx4S(O)p- (where, Rx4 and p have the same meanings as above), or
a 3- to 6-membered ring group containing one or two oxygen atoms;
R5 represents
a cyano group,
a halogen atom,
a C1 to C6 alkoxy group optionally substituted with a substituent A,
a C1 to C6 haloalkoxy group,
a C3 to C8 cycloalkoxy group optionally substituted with a substituent A,
a C2 to C6 alkenyl group optionally substituted with a substituent A,
a C2 to C6 haloalkenyl group,
a C2 to C6 alkynyl group optionally substituted with a substituent A,
a C2 to C6 haloalkynyl group, or
Rx4S(O)p- (where, Rx4 and p have the same meanings as above);
HetA represents Formula (HetA);

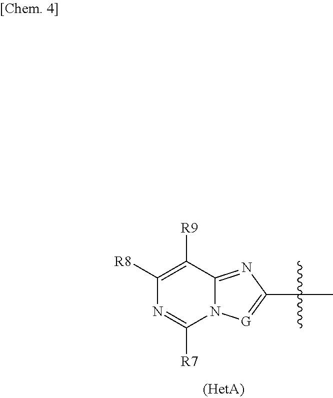

G represents C—R6 or a nitrogen atom;
R6 represents
a hydrogen atom,
a cyano group,
a halogen atom,
a C1 to C6 alkyl group optionally substituted with a substituent A,
a C1 to C6 haloalkyl group,
a C3 to C8 cycloalkyl group optionally substituted with a substituent B,
a C2 to C6 alkenyl group optionally substituted with a substituent A,
a C2 to C6 haloalkenyl group,
a C2 to C6 alkynyl group optionally substituted with a substituent A, or
a C2 to C6 haloalkynyl group;
R7 represents
a hydrogen atom,
a hydroxyl group,
a cyano group,
a halogen atom,
a C1 to C6 alkyl group optionally substituted with a substituent A,
a C1 to C6 haloalkyl group,
a C3 to C8 cycloalkyl group optionally substituted with a substituent B,
a C2 to C6 alkenyl group optionally substituted with a substituent A,
a C2 to C6 haloalkenyl group,
a C2 to C6 alkynyl group optionally substituted with a substituent A,
a C2 to C6 haloalkynyl group,
a C1 to C6 alkoxy group optionally substituted with a substituent A,
a C1 to C6 haloalkoxy group,
a C3 to C8 cycloalkoxy group optionally substituted with a substituent A, or
Rx4S(O)p- (where, Rx4 and p have the same meanings as above);
R8 represents
a hydrogen atom,
a hydroxyl group,
a cyano group,
a halogen atom,
a C1 to C6 alkyl group optionally substituted with a substituent A,
a C1 to C6 haloalkyl group,
a C3 to C8 cycloalkyl group optionally substituted with a substituent B,
a C1 to C6 alkoxy group optionally substituted with a substituent A,
a C1 to C6 haloalkoxy group,
a C3 to C8 cycloalkoxy group optionally substituted with a substituent A, or
Rx4S(O)p- (where, Rx4 and p have the same meanings as above);
R9 represents
a hydrogen atom,
a hydroxyl group,
a cyano group,
a halogen atom,
a C1 to C6 alkyl group optionally substituted with a substituent A,
a C1 to C6 haloalkyl group,
a C3 to C8 cycloalkyl group optionally substituted with a substituent B,
a C1 to C6 alkoxy group optionally substituted with a substituent A,
a C1 to C6 haloalkoxy group,
a C3 to C8 cycloalkoxy group optionally substituted with a substituent A, or
Rx4S(O)p- (where, Rx4 and p have the same meanings as above);
the substituent A is at least one selected from the group consisting of
a hydroxyl group,
a cyano group,
a nitro group,
a halogen atom,
a C3 to C8 cycloalkyl group,
a C1 to C6 alkoxy group,
a C1 to C6 haloalkoxy group,
a C3 to C8 cycloalkoxy group,
a phenyl group optionally substituted with 0 to 5 substituents C,
a 5- or 6-membered aromatic heterocyclic group optionally substituted with 0 to 4 substituents C, and
a 3- to 6-membered ring group containing one or two oxygen atoms;
the substituent B is at least one selected from the group consisting of
a hydroxyl group,
a cyano group,
a nitro group,
a halogen atom,
a C1 to C6 alkyl group,
a C1 to C6 haloalkyl group,
a C3 to C8 cycloalkyl group,
a C1 to C6 alkoxy group,
a C1 to C6 haloalkoxy group,
a C3 to C8 cycloalkoxy group, and
Rx5S(O)p- (where Rx5 represents a hydroxyl group, a halogen atom, a C1 to C6 alkyl group, a C1 to C6 haloalkyl group, a C3 to C8 cycloalkyl group, a C2 to C6 alkenyl group, a C2 to C6 haloalkenyl group, a C2 to C6 alkynyl group, or a C2 to C6 haloalkynyl group, and p has the same meaning as above); and
the substituent C is at least one independently selected from the group consisting of
a cyano group,
a halogen atom,
a C1 to C6 alkyl group,
a C1 to C6 haloalkyl group,
a C3 to C8 cycloalkyl group,
a C1 to C6 alkoxy group,
a C1 to C6 haloalkoxy group,
a C3 to C8 cycloalkoxy group, and
Rx5S(O)p- (where, Rx5 and p have the same meanings as above)].

### Advantageous Effects of Invention

According to the present invention, it is possible to provide a novel compound effective as a pest control agent, particularly, an arthropod pest control agent.

### MODE FOR CARRYING OUT THE INVENTION

Hereinafter, embodiments for carrying out the present invention will be described in detail.

Note that each of the terms used in the claims and the specification is defined as commonly used in the art unless otherwise specified.

Abbreviations used in the present specification are described below.

DMF represents N,N-dimethylformamide, THY represents tetrahydrofuran, Me represents a methyl group, Et represents an ethyl group, Pr represents a propyl group, Bu represents a butyl group, Pent represents a pentyl group, Hex represents a hexyl group, Hept represents a heptyl group, Oct represents an octyl group, Ac represents an acetyl group, Ph represents a phenyl group, Py represents a pyridyl group, c represents cyclo, i represents iso, sec represents secondary, t represents tertiary, ═ represents a double bond, and ≡ represents a triple bond. For Pr, Bu, Pent, and Hex in the columns of the table, when there is no prefix, it means normal.

Hereinafter, definitions of the terms used in the present specification will be described.

The description of Cx to Cy represents having x to y carbon atoms. Here, x and y represent integers, and it is understood that all integers present between x and y are also individually disclosed. For example, C1 to C6 means having 1, 2, 3, 4, 5, or 6 carbon atoms, C2 to C6 means having 2, 3, 4, 5, or 6 carbon atoms, C3 to C8 means having 3, 4, 5, 6, 7, or 8 carbon atoms, and C3 to C6 means having 3, 4, 5, or 6 carbon atoms, respectively.

The term “optionally substituted” means substitution with one substituent or unsubstitution. On the other hand, for example, when the number of substituents is specified as “optionally substituted with 0 to 5”, it is understood that all integers present between 0 and 5 are also individually disclosed. That is, it means that there are no substituents or the number of substituents is 1, 2, 3, 4, or 5. Similarly, “optionally substituted with 0 to 4” means that there are no substituents or the number of substituents is 1, 2, 3, or 4. When the substituent is composed of two or more substituents, the substituents each represent an independent substituent, and may be the same or different.

Examples of the halogen atom include a fluorine atom, a chlorine atom, a bromine atom, and an iodine atom.

The C1 to C6 alkyl group may be linear or branched, and examples thereof include a methyl group, an ethyl group, a propyl group, an isopropyl group, a butyl group, an isobutyl group, a sec-butyl group, a t-butyl group, a pentyl group, an isopentyl group, a 2-methylbutyl group, a neopentyl group, a 1-ethylpropyl group, a hexyl group, a 4-methylpentyl group, a 3-methylpentyl group, a 2-methylpentyl group, a 1-methylpentyl group, a 3,3-dimethylbutyl group, a 2,2-dimethylbutyl group, a 1,1-dimethylbutyl group, a 1,2-dimethylbutyl group, a 1,3-dimethylbutyl group, a 2,3-dimethylbutyl group, a 2-ethylbutyl group, a 1-isopropylpropyl group, a 1,1,2-trimethylpropyl group, and a 1,2,2-trimethylpropyl group.

The C1 to C6 haloalkyl group represents a group in which a hydrogen atom is optionally substituted with one or two or more halogen atoms in the C1 to C6 alkyl group. When substituted with two or more halogen atoms, the halogen atoms may be the same as or different from each other, and the number of substitutions is not particularly limited as long as the halogen atom can be present as a substituent. Specific examples of the C1 to C6 haloalkyl group include a monofluoromethyl group, a difluoromethyl group, a trifluoromethyl group, a monochloromethyl group, a dichloromethyl group, a trichloromethyl group, a monobromomethyl group, a dibromomethyl group, a monoiodomethyl group, a diiodomethyl group, a chlorodifluoromethyl group, a bromodifluoromethyl group, a dichlorofluoromethyl group, a 1-fluoroethyl group, a 2-fluoroethyl group, a 1,1-difluoroethyl group, a 2,2-difluoroethyl group, a 2,2,2-trifluoroethyl group, a 1,1,2,2-tetrafluoroethyl group, a 1,2,2,2-tetrafluoroethyl group, a pentafluoroethyl group, a 2,2,2-trichloroethyl group, a 2-chloro-1,1,2,2-tetrafluoroethyl group, a 3,3-difluoropropyl group, a 3,3,3-trifluoropropyl group, a 3,3,3-trichloropropyl group, a 2,3,3,3-tetrafluoropropyl group, a 2,2,3,3,3-pentafluoropropyl group, a 1,2,2,3,3,3-hexafluoropropyl group, a heptafluoropropyl group, a 1,1,1,3,3,3-hexafluoro-2-methylpropyl group, a 1,1,1-trifluoro-2-methylpropyl group, a heptafluoroisopropyl group, a 2,2,2-trifluoro-1-(trifluoromethyl)ethyl group, a 2,2,3,3,4,4,4-heptafluorobutyl group, a 1,2,2,3,3,4,4,4-octafluorobutyl group, a nonafluorobutyl group, a nonafluoro-sec-butyl group, a 3,3,4,4,5,5,5-heptafluoropentyl group, a 2,3,3,4,4,5,5,5-octafluoropentyl group, a 2,2,3,3,4,4,5,5,5-nonafluoropentyl group, an undecafluoropentyl group, and a tridecafluorohexyl group.

Examples of the C3 to C8 cycloalkyl group include a cyclopropyl group, a cyclobutyl group, a cyclopentyl group, a cyclohexyl group, a cycloheptyl group, and a cyclooctyl group.

The C2 to C6 alkenyl group represents a linear or branched unsaturated hydrocarbon group having one or two or more double bonds. In addition, when there is a geometric isomer, the geometric isomer is only one of an E form and a Z form, or a mixture of the E form and the Z form in an arbitrary ratio, and is not particularly limited as long as the number of carbon atoms is within a specified range. Specific examples of the C2 to C6 alkenyl group include a vinyl group, a 1-propenyl group, an allyl group, a 1-butenyl group, a 2-butenyl group, a 3-butenyl group, a 2-methylallyl group, a 1-pentenyl group, a 2-pentenyl group, a 3-pentenyl group, a 4-pentenyl group, a 3-methyl-2-butenyl group, a 1-hexenyl group, a 2-hexenyl group, a 3-hexenyl group, a 4-hexenyl group, a 5-hexenyl group, a 4-methyl-3-pentenyl group, and a 3-methyl-2-pentenyl group.

The C2 to C6 haloalkenyl group represents a group in which a hydrogen atom is optionally substituted with one or two or more halogen atoms in the C2 to C6 alkenyl group. When substituted with two or more halogen atoms, the halogen atoms may be the same as or different from each other, and the number of substitutions is not particularly limited as long as the halogen atom can be present as a substituent. Specific examples of the C2 to C6 haloalkenyl group include a 2-fluorovinyl group, a 2,2-difluorovinyl group, a 2,2-dichlorovinyl group, a 3-fluoroallyl group, a 3,3-difluoroallyl group, a 2,3,3-trifluoroallyl group, a 3,3-dichloroallyl group, a 4,4-difluoro-3-butenyl group, a 5,5-difluoro-4-pentenyl group, and a 6,6-difluoro-5-hexenyl group.

The C2 to C6 alkynyl group represents a linear or branched unsaturated hydrocarbon group having one or two or more triple bonds. Specific examples of the C2 to C6 alkynyl group include an ethynyl group, a 1-propynyl group, a propargyl group, a 1-butynyl group, a 2-butynyl group, a 3-butynyl group, a 1-pentynyl group, a 2-pentynyl group, a 3-pentynyl group, a 4-pentynyl group, a 1,1-dimethyl-2-propynyl group, a 1-hexynyl group, a 2-hexynyl group, a 3-hexynyl group, a 4-hexynyl group, and a 5-hexynyl group.

The C2 to C6 haloalkynyl group represents a group in which a hydrogen atom is optionally substituted with one or two or more halogen atoms in the C2 to C6 alkynyl group. When substituted with two or more halogen atoms, the halogen atoms may be the same as or different from each other, and the number of substitutions is not particularly limited as long as the halogen atom can be present as a substituent. Specific examples of the C2 to C6 haloalkynyl group include a 2-fluoroethynyl group, a 2-chloroethynyl group, a 2-bromoethynyl group, a 2-iodoethynyl group, a 3,3-difluoro-1-propynyl group, a 3-chloro-3,3-difluoro-1-propynyl group, a 3-bromo-3,3-difluoro-1-propynyl group, a 3,3,3-trifluoro-1-propynyl group, a 4,4-difluoro-1-butynyl group, a 4,4-difluoro-2-butynyl group, a 4-chloro-4,4-difluoro-1-butynyl group, a 4-chloro-4,4-difluoro-2-butynyl group, a 4-bromo-4,4-difluoro-1-butynyl group, a 4-bromo-4,4-difluoro-2-butynyl group, a 4,4,4-trifluoro-1-butynyl group, a 4,4,4-trifluoro-2-butynyl group, a 5,5-difluoro-3-pentynyl group, a 5-chloro-5,5-difluoro-3-pentynyl group, a 5-bromo-5,5-difluoro-3-pentynyl group, a 5,5,5-trifluoro-3-pentynyl group, a 6,6-difluoro-4-hexynyl group, a 6-chloro-6,6-difluoro-4-hexynyl group, a 6-bromo-6,6-difluoro-4-hexynyl group, and a 6,6,6-trifluoro-4-hexynyl group.

The C1 to C6 alkoxy group represents a group in which the C1 to C6 alkyl group is bonded via an oxygen atom. Specific examples of the C1 to C6 alkoxy group include a methoxy group, an ethoxy group, a propyloxy group, an isopropyloxy group, a butoxy group, an isobutoxy group, a sec-butoxy group, a t-butoxy group, a pentyloxy group, an isopentyloxy group, a 1-methylbutoxy group, a 2-methylbutoxy group, a neopentyloxy group, a 1-ethylpropyloxy group, a 1,2-dimethylpropyloxy group, a hexyloxy group, a 1-methylpentyloxy group, a 2-methylpentyloxy group, a 3-methylpentyloxy group, a 4-methylpentyloxy group, a 1,1-dimethylbutoxy group, a 2,2-dimethylbutoxy group, a 3,3-dimethylbutoxy group, a 1,2-dimethylbutoxy group, a 1,3-dimethylbutoxy group, a 2,3-dimethylbutoxy group, a 2-ethylbutoxy group, a 1-isopropylpropyloxy group, a 1,1,2-trimethylpropyloxy group, and a 1,2,2-trimethylpropyloxy group.

The C1 to C6 haloalkoxy group represents a group in which a hydrogen atom is optionally substituted with one or two or more halogen atoms in the C1 to C6 alkoxy group. When substituted with two or more halogen atoms, the halogen atoms may be the same as or different from each other, and the number of substitutions is not particularly limited as long as the halogen atom can be present as a substituent. Specific examples of the C1 to C6 haloalkoxy group include a difluoromethoxy group, a trifluoromethoxy group, a chlorodifluoromethoxy group, a bromodifluoromethoxy group, a 2-fluoroethoxy group, a 2,2-difluoroethoxy group, a 2,2,2-trifluoroethoxy group, a 1,1,2,2-tetrafluoroethoxy group, a 1,2,2,2-tetrafluoroethoxy group, a pentafluoroethoxy group, a 2,2,2-trichloroethoxy group, a 3,3-difluoropropyloxy group, a 3,3,3-trifluoropropyloxy group, a 2,3,3,3-tetrafluoropropyloxy group, a 2,2,3,3,3-pentafluoropropyloxy group, a 1,2,2,3,3,3-hexafluoropropyloxy group, a heptafluoropropyloxy group, a heptafluoroisopropyloxy group, a 2,2,2-trifluoro-1-(trifluoromethyl)-ethoxy group, a 2,2,3,3,4,4,4-heptafluorobutoxy group, a 1,2,2,3,3,4,4,4-octafluorobutoxy group, a nonafluorobutoxy group, a nonafluoro-sec-butoxy group, a 3,3,4,4,5,5,5-heptafluoropentyloxy group, a 2,3,3,4,4,5,5,5-octafluoropentyloxy group, a 2,2,3,3,4,4,5,5,5-nonafluoropentyloxy group, an undecafluoropentyloxy group, and a tridecafluorohexyloxy group.

The C3 to C8 cycloalkoxy group represents a group in which the C3 to C8 cycloalkyl group is bonded via an oxygen atom. Specific examples of the C3 to C8 cycloalkoxy group include a cyclopropyloxy group, a cyclobutoxy group, a cyclopentyloxy group, a cyclohexyloxy group, a cycloheptyloxy group, and a cyclooctyloxy group.

The 5- or 6-membered aromatic heterocyclic group represents a cyclic aromatic substituent selected from the group consisting of a nitrogen atom, an oxygen atom, and a sulfur atom and containing at least one heteroatom as a constituent atom of a ring. Specific examples of the 5- or 6-membered aromatic heterocyclic group include a pyridyl group, a pyridazinyl group, a pyrimidinyl group, a pyrazinyl group, a triazinyl group, a tetrazinyl group, a thienyl group, a thiazolyl group, an isothiazolyl group, a thiadiazolyl group, a furyl group, a pyrrolyl group, an imidazolyl group, a pyrazolyl group, an oxazolyl group, an isoxazolyl group, a triazolyl group, an oxadiazolyl group, a thiadiazolyl group, and a tetrazolyl group.

Specific examples of the 3- to 6-membered ring group containing one or two oxygen atoms include an oxiranyl group, an oxetanyl group, an oxolanyl group, an oxanyl group, a 1,3-dioxolanyl group, a 1,3-dioxanyl group, a 1,4-dioxanyl group, a dihydrofuran-2(3H)-one group, and a tetrahydro-2H-pyran-2-one group.

The pyrazole compound of the present invention includes a compound represented by the following Formula (1-1) or Formula (1-2) and a salt thereof.

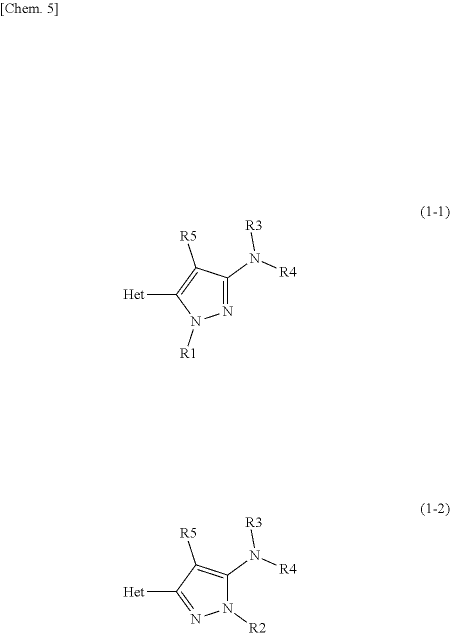

Hereinafter, Formula (1-1) or Formula (1-2) will be described.

In Formula (1-1), R1 represents a hydrogen atom, a C1 to C6 alkyl group optionally substituted with a substituent A, a C1 to C6 haloalkyl group, a C3 to C8 cycloalkyl group optionally substituted with a substituent B, a C2 to C6 alkenyl group optionally substituted with a substituent A, a C2 to C6 haloalkenyl group, a C2 to C6 alkynyl group optionally substituted with a substituent A, a C2 to C6 haloalkynyl group, a phenyl group optionally substituted with 0 to 5 substituents B, a 5- or 6-membered aromatic heterocyclic group optionally substituted with 0 to 4 substituents B, Rx1Rx2NC(═O)— (where Rx1 and Rx2 each independently represent a hydrogen atom, a hydroxyl group, a cyano group, a C1 to C6 alkyl group optionally substituted with a substituent A, a C1 to C6 haloalkyl group, a C3 to C8 cycloalkyl group optionally substituted with a substituent B, a C2 to C6 alkenyl group optionally substituted with a substituent A, a C2 to C6 haloalkenyl group, a C2 to C6 alkynyl group optionally substituted with a substituent A, a C2 to C6 haloalkynyl group, a C1 to C6 alkoxy group, a C1 to C6 haloalkoxy group, a C3 to C8 cycloalkoxy group, a phenyl group optionally substituted with 0 to 5 substituents B, or a 5- or 6-membered aromatic heterocyclic group optionally substituted with 0 to 4 substituents B, or Rx1 and Rx2 together with a nitrogen atom to which they are bonded form an aziridinyl group, an azetidinyl group, a pyrrolidinyl group, a piperidinyl group, a piperazinyl group, a morpholinyl group, a thiomorpholinyl group, a dioxothiomorpholinyl group, a thiazolidinyl group, an azepanyl group, or an azocanyl group), Rx3C(═O)— (where Rx3 represents a hydrogen atom, a hydroxyl group, a halogen atom, a C1 to C6 alkyl group optionally substituted with a substituent A, a C1 to C6 haloalkyl group, a C3 to C8 cycloalkyl group optionally substituted with a substituent B, a C2 to C6 alkenyl group optionally substituted with a substituent A, a C2 to C6 haloalkenyl group, a C2 to C6 alkynyl group optionally substituted with a substituent A, a C2 to C6 haloalkynyl group, a phenyl group optionally substituted with 0 to 5 substituents B, or a 5- or 6-membered aromatic heterocyclic group optionally substituted with 0 to 4 substituents B), Rx3OC(═O)— (where Rx3 has the same meaning as above), Rx4S(O)p- (where Rx4 represents a hydroxyl group, a halogen atom, a C1 to C6 alkyl group optionally substituted with a substituent A, a C1 to C6 haloalkyl group, a C3 to C8 cycloalkyl group optionally substituted with a substituent B, a C2 to C6 alkenyl group optionally substituted with a substituent A, a C2 to C6 haloalkenyl group, a C2 to C6 alkynyl group optionally substituted with a substituent A, a C2 to C6 haloalkynyl group, or a phenyl group optionally substituted with 0 to 5 substituents B, and p represents an integer of 0, 1, or 2), or a 3- to 6-membered ring group containing one or two oxygen atoms.

Among them, R1 is preferably a hydrogen atom, a C1 to C6 alkyl group optionally substituted with a substituent A, a C1 to C6 haloalkyl group, a C3 to C8 cycloalkyl group optionally substituted with a substituent B, a C2 to C6 alkenyl group optionally substituted with a substituent A, a C2 to C6 alkynyl group optionally substituted with a substituent A, or Rx1Rx2NC(═O)— (where Rx1 and Rx2 have the same meanings as above),
R1 is more preferably a hydrogen atom, a C1 to C6 alkyl group optionally substituted with a substituent A, or a C1 to C6 haloalkyl group, and
R1 is particularly preferably a C1 to C6 alkyl group optionally substituted with a substituent A or a C1 to C6 haloalkyl group.

R1 in Formula (1-1) contains a hydrogen atom.

The C1 to C6 alkyl group of the “C1 to C6 alkyl group optionally substituted with a substituent A” in R1 in Formula (1-1) has the same meaning as above, and is preferably a methyl group, an ethyl group, a propyl group, an isopropyl group, a butyl group, an isobutyl group, a sec-butyl group, a pentyl group, or a hexyl group, more preferably a methyl group, an ethyl group, a propyl group, an isopropyl group, a butyl group, an isobutyl group, a sec-butyl group, or a pentyl group, and particularly preferably a methyl group, an ethyl group, a propyl group, an isopropyl group, a butyl group, an isobutyl group, or a sec-butyl group. When having the substituent described in the substituent A, a hydrogen atom in the C1 to C6 alkyl group is optionally substituted with the substituent described in the substituent A.

The “C1 to C6 alkyl group optionally substituted with a substituent A” is preferably a methyl group, an ethyl group, a propyl group, an isopropyl group, a butyl group, an isobutyl group, a sec-butyl group, a pentyl group, a hexyl group, a cyanomethyl group, a 1-cyanoethyl group, a 2-cyanoethyl group, a 3-cyanopropyl group, a 2-cyanopropan-2-yl group, a cyclopropylmethyl group, a cyclobutylmethyl group, a methoxymethyl group, an ethoxymethyl group, a methoxyethyl group, an ethoxyethyl group, an isopropyloxymethyl group, an isopropyloxyethyl group,
a benzyl group, a 1-phenethyl group, a 2-phenethyl group, a 4-cyanophenylmethyl group, a 2-fluorophenylmethyl group, a 3-fluorophenylmethyl group, a 4-fluorophenylmethyl group, a 2-chlorophenylmethyl group, a 3-chlorophenylmethyl group, a 3,5-dichlorophenylmethyl group, a 4-chlorophenylmethyl group, a 2-methylphenylmethyl group, a 3-methylphenylmethyl group, a 4-methylphenylmethyl group, a 2-trifluoromethylphenylmethyl group, a 3-trifluoromethylphenylmethyl group, a 4-trifluoromethylphenylmethyl group, a 2-methoxyphenylmethyl group, a 3-methoxyphenylmethyl group, a 4-methoxyphenylmethyl group, a 4-phenoxyphenylmethyl group, a 4-methanesulfonylphenylmethyl group, a trifluoromethylsulfanylphenylmethyl group,
a pyridin-2-ylmethyl group, a pyridin-2-ylmethyl-N-oxide group, a 3-fluoropyridin-2-ylmethyl group, a 4-fluoropyridin-2-ylmethyl group, a 5-fluoropyridin-2-ylmethyl group, a 6-fluoropyridin-2-ylmethyl group, a 3-chloropyridin-2-ylmethyl group, a 4-chloropyridin-2-ylmethyl group, a 5-chloropyridin-2-ylmethyl group, a 6-chloropyridin-2-ylmethyl group, a 3-bromopyridin-2-ylmethyl group, a 4-bromopyridin-2-ylmethyl group, a 5-bromopyridin-2-ylmethyl group, a 6-bromopyridin-2-ylmethyl group, a 3-methylpyridin-2-ylmethyl group, a 4-methylpyridin-2-ylmethyl group, a 5-methylpyridin-2-ylmethyl group, a 6-methylpyridin-2-ylmethyl group, a 3-trifluoromethylpyridin-2-ylmethyl group, a 4-trifluoromethylpyridin-2-ylmethyl group, a 5-trifluoromethylpyridin-2-ylmethyl group, a 6-trifluoromethylpyridin-2-ylmethyl group, a 3-cyclopropylpyridin-2-ylmethyl group, a 4-cyclopropylpyridin-2-ylmethyl group, a 5-cyclopropylpyridin-2-ylmethyl group, a 6-cyclopropylpyridin-2-ylmethyl group, a 3-methoxypyridin-2-ylmethyl group, a 4-methoxypyridin-2-ylmethyl group, a 5-methoxypyridin-2-ylmethyl group, a 6-methoxypyridin-2-ylmethyl group,
a pyridin-3-ylmethyl group, a 2-fluoropyridin-3-ylmethyl group, a 4-fluoropyridin-3-ylmethyl group, a 5-fluoropyridin-3-ylmethyl group, a 6-fluoropyridin-3-ylmethyl group, a 2-chloropyridin-3-ylmethyl group, a 4-chloropyridin-3-ylmethyl group, a 5-chloropyridin-3-ylmethyl group, a 6-chloropyridin-3-ylmethyl group, a 2-bromopyridin-3-ylmethyl group, a 4-bromopyridin-3-ylmethyl group, a 5-bromopyridin-3-ylmethyl group, a 6-bromopyridin-3-ylmethyl group, a 2-methylpyridin-3-ylmethyl group, a 4-methylpyridin-3-ylmethyl group, a 5-methylpyridin-3-ylmethyl group, a 6-methylpyridin-3-ylmethyl group, a 2-trifluoromethylpyridin-3-ylmethyl group, a 4-trifluoromethylpyridin-3-ylmethyl group, a 5-trifluoromethylpyridin-3-ylmethyl group, a 6-trifluoromethylpyridin-3-ylmethyl group, a 2-methoxypyridin-3-ylmethyl group, a 4-methoxypyridin-3-ylmethyl group, a 5-methoxypyridin-3-ylmethyl group, a 6-methoxypyridin-3-ylmethyl group,
a pyridin-4-ylmethyl group, a 2-fluoropyridin-4-ylmethyl group, a 3-fluoropyridin-4-ylmethyl group, a 2-chloropyridin-4-ylmethyl group, a 3-chloropyridin-4-ylmethyl group, a 2-bromopyridin-4-ylmethyl group, a 3-bromopyridin-4-ylmethyl group, a 2-methylpyridin-4-ylmethyl group, a 3-methylpyridin-4-ylmethyl group, a 2-trifluoromethylpyridin-4-ylmethyl group, a 3-trifluoromethylpyridin-4-ylmethyl group, a 2-methoxypyridin-4-ylmethyl group, a 3-methoxypyridin-4-ylmethyl group,
a pyridazin-3-ylmethyl group, a 4-fluoropyridazin-3-ylmethyl group, a 5-fluoropyridazin-3-ylmethyl group, a 6-fluoropyridazin-3-ylmethyl group, a 4-chloropyridazin-3-ylmethyl group, a 5-chloropyridazin-3-ylmethyl group, a 6-chloropyridazin-3-ylmethyl group, a 4-bromopyridazin-3-ylmethyl group, a 5-bromopyridazin-3-ylmethyl group, a 6-bromopyridazin-3-ylmethyl group, a 4-methylpyridazin-3-ylmethyl group, a 5-methylpyridazin-3-ylmethyl group, a 6-methylpyridazin-3-ylmethyl group, a 4-trifluoromethylpyridazin-3-ylmethyl group, a 5-trifluoromethylpyridazin-3-ylmethyl group, a 6-trifluoromethylpyridazin-3-ylmethyl group, a 4-methoxypyridazin-3-ylmethyl group, a 5-methoxypyridazin-3-ylmethyl group, a 6-methoxypyridazin-3-ylmethyl group,
a pyridazin-4-ylmethyl group, a 3-fluoropyridazin-4-ylmethyl group, a 5-fluoropyridazin-4-ylmethyl group, a 6-fluoropyridazin-4-ylmethyl group, a 3-chloropyridazin-4-ylmethyl group, a 5-chloropyridazin-4-ylmethyl group, a 6-chloropyridazin-4-ylmethyl group, a 3-bromopyridazin-4-ylmethyl group, a 5-bromopyridazin-4-ylmethyl group, a 6-bromopyridazin-4-ylmethyl group, a 3-methylpyridazin-4-ylmethyl group, a 5-methylpyridazin-4-ylmethyl group, a 6-methylpyridazin-4-ylmethyl group, a 3-trifluoromethylpyridazin-4-ylmethyl group, a 5-trifluoromethylpyridazin-4-ylmethyl group, a 6-trifluoromethylpyridazin-4-ylmethyl group, a 3-methoxypyridazin-4-ylmethyl group, a 5-methoxypyridazin-4-ylmethyl group, a 6-methoxypyridazin-4-ylmethyl group,
a pyrimidin-2-ylmethyl group, a 4-fluoropyrimidin-2-ylmethyl group, a 5-fluoropyrimidin-2-ylmethyl group, a 4-chloropyrimidin-2-ylmethyl group, a 5-chloropyrimidin-2-ylmethyl group, a 4-bromo pyrimidin-2-ylmethyl group, a 5-bromopyrimidin-2-ylmethyl group, a 4-methylpyrimidin-2-ylmethyl group, a 5-methylpyrimidin-2-ylmethyl group, a 4-trifluoromethylpyrimidin-2-ylmethyl group, a 5-trifluoromethylpyrimidin-2-ylmethyl group, a 4-methoxypyrimidin-2-ylmethyl group, a 5-methoxypyrimidin-2-ylmethyl group,
a pyrimidin-4-ylmethyl group, a 2-fluoropyrimidin-4-ylmethyl group, a 5-fluoropyrimidin-4-ylmethyl group, a 6-fluoropyrimidin-4-ylmethyl group, a 2-chloropyrimidin-4-ylmethyl group, a 5-chloropyrimidin-4-ylmethyl group, a 6-chloropyrimidin-4-ylmethyl group, a 2-bromopyrimidin-4-ylmethyl group, a 5-bromopyrimidin-4-ylmethyl group, a 6-bromopyrimidin-4-ylmethyl group, a 2-methylpyrimidin-4-ylmethyl group, a 5-methylpyrimidin-4-ylmethyl group, a 6-methylpyrimidin-4-ylmethyl group, a 2-trifluoromethylpyrimidin-4-ylmethyl group, a 5-trifluoromethylpyrimidin-4-ylmethyl group, a 6-trifluoromethylpyrimidin-4-ylmethyl group, a 2-methoxypyrimidin-4-ylmethyl group, a 5-methoxypyrimidin-4-ylmethyl group, a 6-methoxypyrimidin-4-ylmethyl group,
a pyrimidin-5-ylmethyl group, a 2-fluoropyrimidin-5-ylmethyl group, a 4-fluoropyrimidin-5-ylmethyl group, a 6-fluoropyrimidin-5-ylmethyl group, a 2-chloropyrimidin-5-ylmethyl group, a 4-chloropyrimidin-5-ylmethyl group, a 6-chloropyrimidin-5-ylmethyl group, a 2-bromopyrimidin-5-ylmethyl group, a 4-bromopyrimidin-5-ylmethyl group, a 6-bromopyrimidin-5-ylmethyl group, a 2-methylpyrimidin-5-ylmethyl group, a 4-methylpyrimidin-5-ylmethyl group, a 6-methylpyrimidin-5-ylmethyl group, a 2-trifluoromethylpyrimidin-5-ylmethyl group, a 4-trifluoromethylpyrimidin-5-ylmethyl group, a 6-trifluoromethylpyrimidin-5-ylmethyl group, a 2-methoxypyrimidin-5-ylmethyl group, a 4-methoxypyrimidin-5-ylmethyl group, a 6-methoxypyrimidin-5-ylmethyl group,
a pyrazin-2-ylmethyl group, a 3-fluoropyrazin-2-ylmethyl group, a 5-fluoropyrazin-2-ylmethyl group, a 6-fluoropyrazin-2-ylmethyl group, a 3-chloropyrazin-2-ylmethyl group, a 5-chloropyrazin-2-ylmethyl group, a 6-chloropyrazin-2-ylmethyl group, a 3-bromopyrazin-2-ylmethyl group, a 5-bromopyrazin-2-ylmethyl group, a 6-bromopyrazin-2-ylmethyl group, a 3-methylpyrazin-2-ylmethyl group, a 5-methylpyrazin-2-ylmethyl group, a 6-methylpyrazin-2-ylmethyl group, a 3-trifluoromethylpyrazin-2-ylmethyl group, a 5-trifluoromethylpyrazin-2-ylmethyl group, a 6-trifluoromethylpyrazin-2-ylmethyl group, a 3-methoxypyrazin-2-ylmethyl group, a 5-methoxypyrazin-2-ylmethyl group, a 6-methoxypyrazin-2-ylmethyl group, a 1-(pyridin-2-yl)ethyl group, a 1-(3-fluoropyridin-2-yl)ethyl group, a 1-(4-fluoropyridin-2-yl)ethyl group, a 1-(5-fluoropyridin-2-yl)ethyl group, a 1-(6-fluoropyridin-2-yl)ethyl group, a 1-(3-chloropyridin-2-yl)ethyl group, a 1-(4-chloropyridin-2-yl)ethyl group, a 1-(5-chloropyridin-2-yl)ethyl group, a 1-(6-chloropyridin-2-yl)ethyl group, a 1-(3-bromopyridin-2-yl)ethyl group, a 1-(4-bromopyridin-2-yl)ethyl group, a 1-(5-bromopyridin-2-yl)ethyl group, a 1-(6-bromopyridin-2-yl)ethyl group, a 1-(3-methylpyridin-2-yl)ethyl group, a 1-(4-(32-methylpyridin-2-yl)ethyl group, a 1-(5-methylpyridin-2-yl)ethyl group, a 1-(4-methylpyridin-2-yl)ethyl group, a 1-(3-trifluoromethylpyridin-2-yl)ethyl group, a 1-(4-trifluoromethylpyridin-2-yl)ethyl group, a 1-(5-trifluoromethylpyridin-2-yl)ethyl group, a 1-(6-trifluoromethylpyridin-2-yl)ethyl group, a 1-(3-methoxypyridin-2-yl)ethyl group, a 1-(4-methoxypyridin-2-yl)ethyl group, a 1-(5-methoxypyridin-2-yl)ethyl group, a 1-(6-methoxypyridin-2-yl)ethyl group,
a 1-(pyridin-3-yl)ethyl group, a 1-(2-fluoropyridin-3-yl)ethyl group, a 1-(4-fluoropyridin-3-yl)ethyl group, a 1-(5-fluoropyridine-3-yl)ethyl group, a 1-(6-fluoropyridin-3-yl)ethyl group, a 1-(2-chloropyridin-3-yl)ethyl group, a 1-(4-chloropyridin-3-yl)ethyl group, a 1-(5-chloropyridin-3-yl)ethyl group, a 1-(6-chloropyridin-3-yl)ethyl group, a 1-(2-bromopyridin-3-yl)ethyl group, a 1-(4-bromopyridin-3-yl)ethyl group, a 1-(5-bromopyridin-3-yl)ethyl group, a 1-(6-bromopyridin-3-yl)ethyl group, a 1-(2-methylpyridin-3-yl)ethyl group, a 1-(4-methylpyridin-3-yl)ethyl group, a 1-(5-methylpyridin-3-yl)ethyl group, a 1-(6-methylpyridin-3-yl)ethyl group, a 1-(2-trifluoromethylpyridin-3-yl)ethyl group, a 1-(4-trifluoromethylpyridin-3-yl)ethyl group, a 1-(5-trifluoromethylpyridin-3-yl)ethyl group,
a 1-(6-trifluoromethylpyridin-3-yl)ethyl group, a 1-(2-methoxypyridin-3-yl)ethyl group, a 1-(4-methoxypyridin-3-yl)ethyl group, a 1-(5-methoxypyridin-3-yl)ethyl group, a 1-(6-methoxypyridin-3-yl)ethyl group, a 1-(pyridin-4-yl)ethyl group, a 1-(2-fluoropyridin-4-yl)ethyl group, a 1-(3-fluoropyridin-4-yl)ethyl group, a 1-(2-chloropyridin-4-yl)ethyl group, a 1-(3-chloropyridin-4-yl)ethyl group, a 1-(2-bromopyridin-4-yl)ethyl group, a 1-(3-bromopyridin-4-yl)ethyl group, a 1-(2-methylpyridin-4-yl)ethyl group, a 1-(3-methylpyridin-4-yl)ethyl group, a 1-(2-trifluoromethylpyridin-4-yl)ethyl group, a 1-(3-trifluoromethylpyridin-4-yl)ethyl group, a 1-(2-methoxypyridin-4-yl)ethyl group, a 1-(3-methoxypyridin-4-yl)ethyl group,
a 1-(pyridazin-3-yl)ethyl group, a 1-(4-fluoropyridazin-3-yl)ethyl group, a 1-(5-fluoropyridazin-3-yl)ethyl group, a 1-(6-fluoropyridazin-3-yl)ethyl group, a 1-(4-chloropyridazin-3-yl)methyl group, a 1-(5-chloropyridazin-3-yl)ethyl group, a 1-(6-chloropyridazin-3-yl)ethyl group, a 1-(4-bromopyridazin-3-yl)ethyl group, a 1-(5-bromopyridazin-3-yl)ethyl group, a 1-(6-bromopyridazin-3-yl)ethyl group, a 1-(4-methylpyridazin-3-yl)ethyl group, a 1-(5-methylpyridazin-3-yl)ethyl group, a 1-(6-methylpyridazin-3-yl)ethyl group, a 1-(4-trifluoromethylpyridazin-3-yl)ethyl group, a 1-(5-trifluoromethylpyridazin-3-yl)ethyl group, a 1-(6-trifluoromethylpyridazin-3-yl)ethyl group, a 1-(4-methoxypyridazin-3-yl)ethyl group, a 1-(5-methoxypyridazin-3-yl)ethyl group, a 1-(6-methoxypyridazin-3-yl)ethyl group,
a 1-(pyridazin-4-yl)ethyl group, a 1-(3-fluoropyridazin-4-yl)ethyl group, a 1-(5-fluoropyridazin-4-yl)ethyl group, a 1-(6-fluoropyridazine-4-yl)ethyl group, a 1-(3-chloropyridazin-4-yl)ethyl group, a 1-(5-chloropyridazin-4-yl)ethyl group, a 1-(6-chloropyridazin-4-yl)ethyl group, a 1-(3-bromopyridazin-4-yl)ethyl group, a 1-(5-bromopyridazin-4-yl)ethyl group, a 1-(6-bromopyridazin-4-yl)ethyl group, a 1-(6-methylpyridazin-4-yl)ethyl group, a 1-(5-methylpyridazin-4-yl)ethyl group, a 1-(6-methylpyridazin-4-yl)ethyl group, a 1-(3-trifluoromethylpyridazin-4-yl)ethyl group, a 1-(5-trifluoromethylpyridazin-4-yl)ethyl group, a 1-(6-trifluoromethylpyridazin-4-yl)ethyl group, a 1-(3-methoxypyridazin-4-yl)ethyl group, a 1-(5-methoxypyridazin-4-yl)ethyl group, a 1-(6-methoxypyridazin-4-yl)ethyl group,
a 1-(pyrimidin-2-yl)ethyl group, a 1-(4-fluoropyrimidin-2-yl)ethyl group, a 1-(5-fluoropyrimidin-2-yl)ethyl group, a 1-(4-chloropyrimidin-2-yl)ethyl group, a 1-(5-chloropyrimidin-2-yl)ethyl group, a 1-(4-bromopyrimidin-2-yl)ethyl group, a 1-(5-bromopyrimidin-2-yl)ethyl group, a 1-(4-methylpyrimidin-2-yl)ethyl group, a 1-(5-methylpyrimidin-2-yl)ethyl group, a 1-(4-trifluoromethylpyrimidin-2-yl)ethyl group, a 1-(5-trifluoromethylpyrimidin-2-yl)ethyl group, a 1-(4-methoxypyrimidin-2-yl)ethyl group, a 1-(5-methoxypyrimidin-2-yl)ethyl group, a 1-(pyrimidin-4-yl)ethyl group, a 1-(2-fluoropyrimidin-4-yl)ethyl group, a 1-(5-fluoropyrimidin-4-yl)ethyl group, a 1-(6-fluoropyrimidine-4-yl)ethyl group, a 1-(2-chloropyrimidin-4-yl)ethyl group, a 1-(5-chloropyrimidin-4-yl)ethyl group, a 1-(6-chloropyrimidin-4-yl)ethyl group, a 1-(2-bromopyrimidin-4-yl)ethyl group, a 1-(5-bromopyrimidin-4-yl)ethyl group, a 1-(6-bromopyrimidin-4-yl)ethyl group, a 1-(2-methylpyrimidin-4-yl)ethyl group, a 1-(5-methylpyrimidin-4-yl)ethyl group, a 1-(6-methylpyrimidin-4-yl)ethyl group, a 1-(2-trifluoromethylpyrimidin-4-yl)ethyl group, a 1-(5-trifluoromethylpyrimidin-4-yl)ethyl group, a 1-(6-trifluoromethylpyrimidin-4-yl)ethyl group, a 1-(2-methoxypyrimidin-4-yl)ethyl group, a 1-(5-methoxypyrimidin-4-yl)ethyl group, a 1-(6-methoxypyrimidin-4-yl)ethyl group,
a 1-(pyrimidin-5-yl)ethyl group, a 1-(2-fluoropyrimidin-5-yl)ethyl group, a 1-(4-fluoropyrimidin-5-yl)ethyl group, a 1-(6-fluoropyrimidine-5-yl)ethyl group, a 1-(2-chloropyrimidin-5-yl)ethyl group, a 1-(4-chloropyrimidin-5-yl)ethyl group, a 1-(6-chloropyrimidin-5-yl)ethyl group, a 1-(2-bromopyrimidin-5-yl)ethyl group, a 1-(4-bromopyrimidin-5-yl)ethyl group, a 1-(6-bromopyrimidin-5-yl)ethyl group, a 1-(2-methylpyrimidin-5-yl)ethyl group, a 1-(4-methylpyrimidin-5-yl)ethyl group, a 1-(6-methylpyrimidin-5-yl)ethyl group, a 1-(2-trifluoromethylpyrimidin-5-yl)ethyl group, a 1-(4-trifluoromethylpyrimidin-5-yl)ethyl group, a 1-(6-trifluoromethylpyrimidin-5-yl)ethyl group, a 1-(2-methoxypyrimidin-5-yl)ethyl group, a 1-(4-methoxypyrimidin-5-yl)ethyl group, a 1-(6-methoxypyrimidin-5-yl)ethyl group,
a 1-(pyrazin-2-yl)ethyl group, a 1-(3-fluoropyrazin-2-yl)ethyl group, a 1-(5-fluoropyrazin-2-yl)ethyl group, a 1-(6-fluoropyrazine-2-yl)ethyl group, a 1-(3-chloropyrazin-2-yl)ethyl group, a 1-(5-chloropyrazin-2-yl)ethyl group, a 1-(6-chloropyrazin-2-yl)ethyl group, a 1-(3-bromopyrazin-2-yl)ethyl group, a 1-(5-bromopyrazin-2-yl)ethyl group, a 1-(6-bromopyrazin-2-yl)ethyl group, a 1-(3-methylpyrazin-2-yl)ethyl group, a 1-(5-methylpyrazin-2-yl)ethyl group, a 1-(6-methylpyrazin-2-yl)ethyl group, a 1-(3-trifluoromethylpyrazin-2-yl)ethyl group, a 1-(5-trifluoromethylpyrazin-2-yl)ethyl group, a 1-(6-trifluoromethylpyrazin-2-yl)ethyl group, a 1-(3-methoxypyrazin-2-yl)ethyl group, a 1-(5-methoxypyrazin-2-yl)ethyl group, a 1-(6-methoxypyrazin-2-yl)ethyl group,
a 3-methyl-1,2,4-oxadiazolyl group, a 5-fluoro-3-methyl-1,2,4-oxadiazolyl group, a 5-chloro-3-methyl-1,2,4-oxadiazolyl group, a 5-bromo-3-methyl-1,2,4-oxadiazolyl group, a 5-methyl-3-methyl-1,2,4-oxadiazolyl group, a 5-trifluoromethyl-3-methyl-1,2,4-oxadiazolyl group, a 5-methoxy-3-methyl-1,2,4-oxadiazolyl group, a thiazol-2-ylmethyl group, a thiazol-4-ylmethyl group, a thiazol-5-ylmethyl group,
a tetrahydrofuran-2-ylmethyl group, a tetrahydrofuran-3-ylmethyl group, a tetrahydro-2H-pyran-2-ylmethyl group, a tetrahydro-2H-pyran-4-ylmethyl group, a 1,3-dioxolan-2-ylmethyl group, or a 3-(dihydrofuran-2(3H)-one)methyl group,
more preferably a methyl group, an ethyl group, a propyl group, an isopropyl group, a butyl group, an isobutyl group, a sec-butyl group, a pentyl group, a cyanomethyl group, a 1-cyanoethyl group, a 2-cyanoethyl group, a cyclopropylmethyl group, a methoxymethyl group, an ethoxymethyl group, a methoxyethyl group, an ethoxyethyl group, an isopropyloxymethyl group, a benzyl group, a 1-phenethyl group, a 2-phenethyl group, a 4-cyanophenylmethyl group, a 2-fluorophenylmethyl group, a 3-fluorophenylmethyl group, a 4-fluorophenylmethyl group, a 2-chlorophenylmethyl group, a 3-chlorophenylmethyl group, a 3,5-dichlorophenylmethyl group, a 4-chlorophenylmethyl group, a 2-methylphenylmethyl group, a 3-methylphenylmethyl group, a 4-methylphenylmethyl group, a 2-trifluoromethylphenylmethyl group, a 3-trifluoromethylphenylmethyl group, a 4-trifluoromethylphenylmethyl group, a 3-cyclopropylpyridin-2-ylmethyl group, a 4-cyclopropylpyridin-2-ylmethyl group, a 5-cyclopropylpyridin-2-ylmethyl group, a 6-cyclopropylpyridin-2-ylmethyl group, a 2-methoxyphenylmethyl group, a 3-methoxyphenylmethyl group, a 4-methoxyphenylmethyl group, a 4-phenoxyphenylmethyl group, a 4-methanesulfonylphenylmethyl group, a 4-trifluoromethylsulfanylphenylmethyl group, a 4-trifluorosulfinyiphenylmethyl group, a 4-trifluorosulfonylphenylmethyl group, a pyridin-2-ylmethyl group, a pyridin-2-ylmethyl-N-oxide group, a 3-chloropyridin-2-ylmethyl group, a 4-chloropyridin-2-ylmethyl group, a 5-chloropyridin-2-ylmethyl group, a 6-chloropyridin-2-ylmethyl group, a 3-bromopyridin-2-ylmethyl group, a 4-bromopyridin-2-ylmethyl group, a 5-bromopyridin-2-ylmethyl group, a 6-bromopyridin-2-ylmethyl group, a 3-methylpyridin-2-ylmethyl group, a 4-methylpyridin-2-ylmethyl group, a 5-methylpyridin-2-ylmethyl group, a 6-methylpyridin-2-ylmethyl group, a 3-trifluoromethylpyridin-2-ylmethyl group, a 4-trifluoromethylpyridin-2-ylmethyl group, a 5-trifluoromethylpyridine-2-ylmethyl group,
a 6-trifluoromethylpyridin-2-ylmethyl group, a 3-cyclopropylpyridin-2-ylmethyl group, a 4-cyclopropylpyridin-2-ylmethyl group, a 5-cyclopropylpyridin-2-ylmethyl group, a 6-cyclopropylpyridin-2-ylmethyl group, a 3-methoxypyridin-2-ylmethyl group, a 4-methoxypyridin-2-ylmethyl group, a 5-methoxypyridin-2-ylmethyl group, a 6-methoxypyridin-2-ylmethyl group, a pyridin-3-ylmethyl group, a 2-chloropyridin-3-ylmethyl group, a 4-chloropyridin-3-ylmethyl group, a 5-chloropyridin-3-ylmethyl group, a 6-chloropyridin-3-ylmethyl group, a 2-methylpyridin-3-ylmethyl group, a 4-methylpyridin-3-ylmethyl group, a 5-methylpyridin-3-ylmethyl group, a 6-methylpyridin-3-ylmethyl group, a 2-trifluoromethylpyridin-3-ylmethyl group, a 4-trifluoromethylpyridin-3-ylmethyl group, a 5-trifluoromethylpyridin-3-ylmethyl group, a 6-trifluoromethylpyridin-3-ylmethyl group, a pyridin-4-ylmethyl group, a 2-chloropyridin-4-ylmethyl group, a 3-chloropyridin-4-ylmethyl group, a 2-methylpyridin-4-ylmethyl group, a 3-methylpyridin-4-ylmethyl group, a 2-trifluoromethylpyridin-4-ylmethyl group, a 3-trifluoromethylpyridin-4-ylmethyl group, a pyridazin-3-ylmethyl group, a 4-chloropyridazin-3-ylmethyl group, a 5-chloropyridazin-3-ylmethyl group, a 6-chloropyridazin-3-ylmethyl group, a 4-methylpyridazin-3-ylmethyl group, a 5-methylpyridazin-3-ylmethyl group, a 6-methylpyridazin-3-ylmethyl group, a 4-trifluoromethylpyridazin-3-ylmethyl group, a 5-trifluoromethylpyridazin-3-ylmethyl group, a 6-trifluoromethylpyridazin-3-ylmethyl group, a pyridazin-4-ylmethyl group, a 3-chloropyridazin-4-ylmethyl group, a 5-chloropyridazin-4-ylmethyl group, a 6-chloropyridazin-4-ylmethyl group, a 3-methylpyridazin-4-ylmethyl group, a 5-methylpyridazin-4-ylmethyl group, a 6-methylpyridazin-4-ylmethyl group, a 3-trifluoromethylpyridazin-4-ylmethyl group, a 5-trifluoromethylpyridazin-4-ylmethyl group, a 6-trifluoromethylpyridazin-4-ylmethyl group, a 3-methoxypyridazin-4-ylmethyl group, a pyrimidin-2-ylmethyl group,
a 4-chloropyrimidin-2-ylmethyl group, a 5-chloropyrimidin-2-ylmethyl group, a 4-methylpyrimidin-2-ylmethyl group, a 5-methylpyrimidin-2-ylmethyl group, a 4-trifluoromethylpyrimidin-2-ylmethyl group, a 5-trifluoromethylpyrimidin-2-ylmethyl group, a pyrimidin-4-ylmethyl group, a 2-chloropyrimidin-4-ylmethyl group, a 5-chloropyrimidin-4-ylmethyl group, a 6-chloropyrimidin-4-ylmethyl group, a 2-methylpyrimidin-4-ylmethyl group, a 5-methylpyrimidin-4-ylmethyl group, a 6-methylpyrimidin-4-ylmethyl group, a 2-trifluoromethylpyrimidin-4-ylmethyl group, a 5-trifluoromethylpyrimidin-4-ylmethyl group, a 6-trifluoromethylpyrimidin-4-ylmethyl group, a pyrimidin-5-ylmethyl group, a 2-chloropyrimidin-5-ylmethyl group, a 4-chloropyrimidin-5-ylmethyl group, a 6-chloropyrimidin-5-ylmethyl group, a 2-methylpyrimidin-5-ylmethyl group, a 4-methylpyrimidin-5-ylmethyl group, a 6-methylpyrimidin-5-ylmethyl group, a 2-trifluoromethylpyrimidin-5-ylmethyl group, a 4-trifluoromethylpyrimidin-5-ylmethyl group, a 6-trifluoromethylpyrimidin-5-ylmethyl group, a pyrazin-2-ylmethyl group, a 3-chloropyrazin-2-ylmethyl group, a 5-chloropyrazin-2-ylmethyl group, a 6-chloropyrazin-2-ylmethyl group, a 3-methylpyrazin-2-ylmethyl group, a 5-methylpyrazin-2-ylmethyl group, a 6-methylpyrazin-2-ylmethyl group, a 3-trifluoromethylpyrazin-2-ylmethyl group, a 5-trifluoromethylpyrazin-2-ylmethyl group, a 6-trifluoromethylpyrazin-2-ylmethyl group, a 1-(pyridin-2-yl)ethyl group, a 1-(3-chloropyridin-2-yl)ethyl group, a 1-(4-chloropyridin-2-yl)ethyl group, a 1-(5-chloropyridin-2-yl)ethyl group, a 1-(6-chloropyridin-2-yl)ethyl group, a 1-(3-methylpyridin-2-yl)ethyl group, a 1-(4-methylpyridin-2-yl)ethyl group, a 1-(5-methylpyridin-2-yl)ethyl group, a 1-(6-methylpyridin-2-yl)ethyl group, a 1-(3-trifluoromethylpyridin-2-yl)ethyl group, a 1-(4-trifluoromethylpyridin-2-yl)ethyl group, a 1-(5-trifluoromethylpyridin-2-yl)ethyl group, a 1-(6-trifluoromethylpyridin-2-yl)ethyl group,
a 1-(pyridin-3-yl)ethyl group, a 1-(2-chloropyridin-3-yl)ethyl group, a 1-(4-chloropyridin-3-yl)ethyl group, a 1-(5-chloropyridin-3-yl)ethyl group, a 1-(6-chloropyridin-3-yl)ethyl group, a 1-(2-methylpyridin-3-yl)ethyl group, a 1-(4-methylpyridin-3-yl)ethyl group, a 1-(5-methylpyridin-3-yl)ethyl group, a 1-(6-methylpyridin-3-yl)ethyl group, a 1-(2-trifluoromethylpyridin-3-yl)ethyl group, a 1-(4-trifluoromethylpyridin-3-yl)ethyl group, a 1-(5-trifluoromethylpyridin-3-yl)ethyl group, a 1-(6-trifluoromethylpyridin-3-yl)ethyl group, a 1-(pyridin-4-yl)ethyl group, a 1-(2-chloropyridin-4-yl)ethyl group, a 1-(3-chloro pyridin-4-yl)ethyl group, a 1-(2-methylpyridin-4-yl)ethyl group, a 1-(3-methylpyridin-4-yl)ethyl group, a 1-(2-trifluoromethylpyridin-4-yl)ethyl group, a 1-(3-trifluoromethylpyridin-4-yl)ethyl group, a 1-(pyridazin-3-yl)ethyl group, a 1-(4-chloropyridazin-3-yl)methyl group, a 1-(5-chloropyridazin-3-yl)ethyl group, a 1-(6-chloropyridazin-3-yl)ethyl group, a 1-(4-methylpyridazin-3-yl)ethyl group, a 1-(5-methylpyridazin-3-yl)ethyl group, a 1-(6-methylpyridazin-3-yl)ethyl group, a 1-(4-trifluoromethylpyridazin-3-yl)ethyl group, a 1-(5-trifluoromethylpyridazin-3-yl)ethyl group, a 1-(6-trifluoromethylpyridazin-3-yl)ethyl group, a 1-(pyridazin-4-yl)ethyl group, a 1-(3-chloropyridazin-4-yl)ethyl group, a 1-(5-chloropyridazin-4-yl)ethyl group, a 1-(6-chloropyridazin)-4-yl)ethyl group, a 1-(3-methylpyridazin-4-yl)ethyl group, a 1-(5-methylpyridazin-4-yl)ethyl group, a 1-(6-methylpyridazin-4-yl)ethyl group, a 1-(3-trifluoromethylpyridazin-4-yl)ethyl group, a 1-(5-trifluoromethylpyridazin-4-yl)ethyl group, a 1-(6-trifluoromethylpyridazin-4-yl)ethyl group, a 1-(pyrimidin-2-yl)ethyl group, a 1-(4-chloropyrimidin)-2-yl)ethyl group, a 1-(5-chloropyrimidin-2-yl)ethyl group, a 1-(4-methylpyrimidin-2-yl)ethyl group, a 1-(5-methylpyrimidin-2-yl)ethyl group, a 1-(4-trifluoromethylpyrimidin-2-yl)ethyl group, a 1-(5-trifluoromethylpyrimidin-2-yl)ethyl group, a 1-(pyrimidin-4-yl)ethyl group, a 1-(2-chloropyrimidin-4-yl)ethyl group, a 1-(5-chloropyrimidin-4)-yl)ethyl group, a 1-(6-chloropyrimidin-4-yl)ethyl group, a 1-(2-methylpyrimidin-4-yl)ethyl group, a 1-(5-methylpyrimidin-4-yl)ethyl group, a 1-(6-methylpyrimidin-4-yl)ethyl group, a 1-(2-trifluoromethylpyrimidin-4-yl)ethyl group, a 1-(5-trifluoromethylpyrimidin-4-yl)ethyl group, a 1-(6-trifluoromethylpyrimidin-4-yl)ethyl group, a 1-(pyrimidin-5-yl)ethyl group, a 1-(2-chloropyrimidin-5-yl)ethyl group, a 1-(4-chloropyrimidin-5-yl)ethyl group, a 1-(6-chloropyrimidin-5-yl)ethyl group, a 1-(2-methylpyrimidin-5-yl)ethyl group, a 1-(4-methylpyrimidin-5-yl)ethyl group, a 1-(6-methylpyrimidin-5-yl)ethyl group, a 1-(2-trifluoromethylpyrimidin-5-yl)ethyl group, a 1-(4-trifluoromethylpyrimidin-5-yl)ethyl group, a 1-(6-trifluoromethylpyrimidin-5-yl)ethyl group, a 1-(pyrazin-2-yl)ethyl group, a 1-(3-chloropyrazin-2-yl)ethyl group, a 1-(5-chloropyrazin-2-yl)ethyl group, a 1-(6-chloropyrazin-2-yl)ethyl group, a 1-(3-methylpyrazin-2-yl)ethyl group, a 1-(5-methylpyrazin-2-yl)ethyl group, a 1-(6-methylpyrazin-2-yl)ethyl group, a 1-(3-trifluoromethylpyrazin-2-yl)ethyl group, a 1-(5-trifluoromethylpyrazin-2-yl)ethyl group, a 1-(6-trifluoromethylpyrazin-2-yl)ethyl group, a 3-methyl-1,2,4-oxadiazolyl group, a 5-trifluoromethyl-3-methyl-1,2,4-oxadiazolyl group, a thiazol-2-ylmethyl group, a thiazol-4-ylmethyl group, a thiazol-5-ylmethyl group, a propan-2-one group, a methoxycarbonylmethyl group, a methoxycarbonylethyl group, an ethoxycarbonylmethyl group, an ethoxycarbonylethyl group, a dimethylaminocarbonylmethyl group, an ethylmethylamino carbonylmethyl group, a methylsulfanylmethyl group, a methylsulfinylmethyl group, a methylsulfonylmethyl group, an ethylsulfanylmethyl group, an ethylsulfinylmethyl group, an ethylsulfonylmethyl group, a trifluoromethylsulfanylmethyl group, a trifluoromethylsulfinylmethyl group, a trifluorosulfonylmethyl group, a tetrahydrofuran-2-ylmethyl group, a tetrahydrofuran-3-ylmethyl group, a tetrahydro-2H-pyran-2-ylmethyl group, a tetrahydro-2H-pyran-4-ylmethyl group, a 1,3-dioxolan-2-ylmethyl group, or a 3-(dihydrofuran-2(3H)-one)methyl group,
still more preferably a methyl group, an ethyl group, a propyl group, an isopropyl group, a butyl group, an isobutyl group, a sec-butyl group, a cyanomethyl group, a 2-cyanoethyl group, a cyclopropylmethyl group, a methoxymethyl group, an ethoxymethyl group, a methoxyethyl group, an ethoxyethyl group, or a pyrimidin-5-ylmethyl group, and
particularly preferably a pyrimidin-5-ylmethyl group.

The “C1 to C6 haloalkyl group” in R1 in Formula (1-1) has the same meaning as above, and is preferably a difluoromethyl group, a trifluoromethyl group, a 2,2-difluoroethyl group, a 2,2,2-trifluoroethyl group, a 1,2,2,2-tetrafluoroethyl group, a 1,1,2,2,2-pentafluoroethyl group, a 2,2,3,3,3-pentafluoropropyl group, a 1,1,2,2,3,3,3-heptafluoropropyl group, a 2,2,3,3,4,4,4-heptafluorobutyl group, or a 2,2,3,3,4,4,5,5,5-nonafluoropentyl group, more preferably a difluoromethyl group, a 2,2,2-trifluoroethyl group, a 1,1,2,2,2-pentafluoroethyl group, a 1,1,2,2,3,3,3-heptafluoropropyl group, a 2,2,3,3,4,4,4-heptafluorobutyl group, or a 2,2,3,3,4,4,5,5,5-nonafluoropentyl group, still more preferably a difluoromethyl group, a 2,2,2-trifluoroethyl group, a 2,2,3,3,3-pentafluoropropyl group, or a 2,2,3,3,4,4,4-heptafluorobutyl group, and particularly preferably a 2,2,2-trifluoroethyl group.

The C3 to C8 cycloalkyl group of the “C3 to C8 cycloalkyl group optionally substituted with a substituent B” in R1 in Formula (1-1) has the same meaning as above, and is preferably a cyclopropyl group, a cyclobutyl group, a cyclopentyl group, or a cyclohexyl group, more preferably a cyclopropyl group or a cyclobutyl group, and particularly preferably a cyclopropyl group. When having a substituent B, one hydrogen atom in the C3 to C8 cycloalkyl group is substituted with the substituent B. The “C3 to C8 cycloalkyl group optionally substituted with a substituent B” is preferably a cyclopropyl group, a cyclobutyl group, a cyclopentyl group, a cyclohexyl group, a 1-(cyclopropane-1-carbonitrile) group, a 2-(cyclopropane-1-carbonitrile) group, a 1-(1-nitrocyclopropyl) group, a 1-(2-nitrocyclopropyl) group, a 1-(1-cyclopropylcyclopropyl) group, a 1-(2-cyclopropylcyclopropyl) group, a 1-(2-methoxycyclopropyl) group, a 1-(2-ethoxycyclopropyl) group, a 1-(1-phenylcyclopropyl) group, a 1-(2-phenylcyclopropyl) group, a 1-(cyclopropylethane-1-one) group, a 2-(cyclopropylethan-1-one) group, a 1-(1-methoxycarbonylcyclopropyl) group, a 1-(2-methoxycarbonylcyclopropyl) group, a 1-(1-ethoxycarbonylcyclopropyl) group, or a 1-(2-ethoxycarbonylcyclopropyl) group,
more preferably a cyclopropyl group, a cyclobutyl group, a 1-(cyclopropane-1-carbonitrile) group, a 2-(cyclopropane-1-carbonitrile) group, a 1-(1-nitrocyclopropyl) group, a 1-(2-nitrocyclopropyl) group, a 1-(1-methoxycarbonylcyclopropyl) group, a 1-(2-methoxycarbonylcyclopropyl) group, a 1-(1-ethoxycarbonylcyclopropyl) group, or a 1-(2-ethoxycarbonylcyclopropyl) group,
still more preferably a cyclopropyl group, a 1-(cyclopropane-1-carbonitrile) group, or a 1-(1-methoxycarbonylcyclopropyl) group, and
particularly preferably a cyclopropyl group or a 1-(cyclopropane-1-carbonitrile) group.

The C2 to C6 alkenyl group of the “C2 to C6 alkenyl group optionally substituted with a substituent A” in R1 in Formula (1-1) has the same meaning as above, and is preferably a vinyl group, a 1-propenyl group, an allyl group, a 2-methylallyl group, a 1-butenyl group, a 2-butenyl group, or a 3-butenyl group, more preferably a vinyl group, a 1-propenyl group, an allyl group, a 2-methylallyl group, or a 3-butenyl group, and particularly preferably an allyl group, a 2-methylallyl group, or a 3-butenyl group. When having the substituent described in the substituent A, a hydrogen atom in the C2 to C6 alkenyl group is optionally substituted with the substituent described in the substituent A.

The “C2 to C6 haloalkenyl group” in R1 in Formula (1-1) has the same meaning as above, is preferably a 2-fluorovinyl group, a 2,2-difluorovinyl group, a 3-fluoroallyl group, or a 3,3-difluoroallyl group, more preferably a 3-fluoroallyl group or a 3,3-difluoroallyl group, and particularly preferably a 3,3-difluoroallyl group.

The C2 to C6 alkynyl group of the “C2 to C6 alkynyl group optionally substituted with a substituent A” in R1 in Formula (1-1) has the same meaning as above, and is preferably an ethynyl group, a 1-propynyl group, a propargyl group, a 1-butynyl group, a 2-butynyl group, or a 3-butynyl group, more preferably an ethynyl group, a 1-propynyl group, a propargyl group, or a 2-butynyl group, and particularly preferably a propargyl group or a 2-butynyl group. When having a substituent A, one hydrogen atom in the C2 to C6 alkynyl group is substituted with the substituent A.

The “C2 to C6 haloalkynyl group” in R1 in Formula (1-1) has the same meaning as above, and is preferably a 3,3-difluoro-1-propynyl group, a 3,3,3-trifluoro-1-propynyl group, a 4,4-difluoro-1-butynyl group, a 4,4-difluoro-2-butynyl group, a 4,4,4-trifluoro-1-butynyl group, or a 4,4,4-trifluoro-2-butynyl group, more preferably a 3,3-difluoro-1-propynyl group, a 3,3,3-trifluoro-1-propynyl group, or a 4,4,4-trifluoro-1-butynyl group, and particularly preferably a 3,3-difluoro-1-propynyl group or a 3,3,3-trifluoro-1-propynyl group.

In the “phenyl group optionally substituted with 0 to 5 substituents B” in R1 in Formula (1-1), hydrogen atoms in the phenyl group are optionally substituted with 1 to 5 independent substituents B.

The “phenyl group optionally substituted with 0 to 5 substituents B” is preferably a phenyl group, a cyanophenyl group, a fluorophenyl group, a chlorophenyl group, a tolyl group, a methoxyphenyl group, a phenoxyphenyl group, a methylsulfanylphenyl group, a methylsulfinylphenyl group, a methylsulfonylphenyl group, a trifluoromethylsulfanylphenyl group, a trifluoromethylsulfinylphenyl group, or a trifluoromethylsulfonylphenyl group, more preferably a phenyl group, a cyanophenyl group, a fluorophenyl group, a chlorophenyl group, a tolyl group, a methoxyphenyl group, a methylsulfonylphenyl group, a trifluoromethylsulfanylphenyl group, a trifluoromethylsulfinylphenyl group, or a trifluoromethylsulfonylphenyl group, and particularly preferably a phenyl group.

The 5- or 6-membered aromatic heterocyclic group of the “5- or 6-membered aromatic heterocyclic group optionally substituted with 0 to 4 substituents B” in R1 in Formula (1-1) has the same meaning as above, and is preferably a pyridyl group, a pyridazinyl group, a pyrimidinyl group, a pyrazinyl group, a thienyl group, a thiazolyl group, an isothiazolyl group, an imidazolyl group, a pyrazolyl group, an oxazolyl group, an isoxazolyl group, a triazolyl group, an oxadiazolyl group, a thiadiazolyl group, or a tetrazolyl group, more preferably a pyridyl group, a pyridazinyl group, a pyrimidinyl group, a pyrazinyl group, a thienyl group, a thiazolyl group, an imidazolyl group, a pyrazolyl group, an oxazolyl group, an isoxazolyl group, a triazolyl group, or an oxadiazolyl group, and particularly preferably a pyridyl group or a pyrimidinyl group.

The “5- or 6-membered aromatic heterocyclic group optionally substituted with 0 to 4 substituents B” is preferably a pyridyl group, a 2-cyanopyridyl group, a 3-cyanopyridyl group, a 4-cyanopyridyl group, a 2-fluoropyridyl group, a 3-fluoropyridyl group, a 4-fluoropyridyl group, a 2-chloropyridyl group, a 3-chloropyridyl group, a 4-chloropyridyl group, a 2-bromopyridyl group, a 3-bromopyridyl group, a 4-bromopyridyl group, a 2-iodopyridyl group, a 3-iodopyridyl group, a 4-iodopyridyl group, a 2-methylpyridyl group, a 3-methylpyridyl group, a 4-methylpyridyl group, a 2-trifluoromethylpyridyl group, a 3-trifluoromethylpyridyl group, a 4-trifluoromethylpyridyl group, a 2-methoxypyridyl group, a 3-methoxypyridyl group, a 4-methoxypyridyl group, a pyridazinyl group, a 3-cyanopyridazinyl group, a 4-cyanopyridazinyl group, a 3-fluoropyridazinyl group, a 4-fluoropyridazinyl group, a 3-chloropyridazinyl group, a 4-chloropyridazinyl group, a 3-bromopyridazinyl group, a 4-bromopyridazinyl group, a 3-iodopyridazinyl group, a 4-iodopyridazinyl group, a 3-methylpyridazinyl group, a 4-methylpyridazinyl group, a 3-trifluoromethylpyridazinyl group, a 4-trifluoromethylpyridazinyl group, a 3-methoxypyridazinyl group, a 4-methoxypyridazinyl group, a pyrimidinyl group, a 2-cyanopyrimidinyl group, a 4-cyanopyrimidinyl group, a 5-cyanopyrimidinyl group, a 2-fluoropyrimidinyl group, a 4-fluoropyrimidinyl group, a 5-fluoropyrimidinyl group, a 2-chloropyrimidinyl group, a 4-chloropyrimidinyl group, a 5-chloropyrimidinyl group, a 2-bromopyrimidinyl group, a 4-bromopyrimidinyl group, a 5-bromopyrimidinyl group, a 2-iodopyrimidinyl group, a 4-iodopyrimidinyl group, a 5-iodopyrimidinyl group, a 2-methylpyrimidinyl group, a 4-methylpyrimidinyl group, a 5-methylpyrimidinyl group, a 2-trifluoromethylpyrimidinyl group, a 4-trifluoromethylpyrimidinyl group, a 5-trifluoromethylpyrimidinyl group, a 2-methoxypyrimidinyl group, a 4-methoxypyrimidinyl group, a 5-methoxypyrimidinyl group, a pyrazinyl group, a 2-cyanopyrazinyl group, a 2-fluoropyrazinyl group, a 2-chloropyrazinyl group, a 2-bromopyrazinyl group, a 2-iodopyrazinyl group, a 2-methylpyrazinyl group, a 2-trifluoromethylpyrazinyl group, or a 2-methoxypyrazinyl group,
more preferably a pyridyl group, a 2-cyanopyridyl group, a 3-cyanopyridyl group, a 4-cyanopyridyl group, a 2-fluoropyridyl group, a 3-fluoropyridyl group, a 4-fluoropyridyl group, a 2-chloropyridyl group, a 3-chloropyridyl group, a 4-chloropyridyl group, a 2-bromopyridyl group, a 3-bromopyridyl group, a 4-bromopyridyl group, a 2-iodopyridyl group, a 3-iodopyridyl group, a 4-iodopyridyl group, a 2-methylpyridyl group, a 3-methylpyridyl group, a 4-methylpyridyl group, a 2-trifluoromethylpyridyl group, a 3-trifluoromethylpyridyl group, a 4-trifluoromethylpyridyl group, a 2-methoxypyridyl group, a 3-methoxypyridyl group, a 4-methoxypyridyl group, a pyrimidyl group, a 2-cyanopyrimidinyl group, a 4-cyanopyrimidinyl group, a 5-cyanopyrimidinyl group, a 2-fluoropyrimidinyl group, a 4-fluoropyrimidinyl group, a 5-fluoropyrimidinyl group, a 2-chloropyrimidinyl group, a 4-chloropyrimidinyl group, a 5-chloropyrimidinyl group, a 2-bromopyrimidinyl group, a 4-bromopyrimidinyl group, a 5-bromopyrimidinyl group, a 2-iodopyrimidinyl group, a 4-iodopyrimidinyl group, a 5-iodopyrimidinyl group, a 2-methylpyrimidinyl group, a 4-methylpyrimidinyl group, a 5-methylpyrimidinyl group, a 2-trifluoromethylpyrimidinyl group, a 4-trifluoromethylpyrimidinyl group, a 5-trifluoromethylpyrimidinyl group, a 2-methoxypyrimidinyl group, a 4-methoxypyrimidinyl group, or a 5-methoxypyrimidinyl group, and
particularly preferably a pyridyl group, a 3-trifluoromethylpyridyl group, or a pyrimidyl group.

Each term of Rx1Rx2NC(═O)— (where Rx1 and Rx2 each independently represent a hydrogen atom, a hydroxyl group, a cyano group, a C1 to C6 alkyl group optionally substituted with a substituent A, a C1 to C6 haloalkyl group, a C3 to C8 cycloalkyl group optionally substituted with a substituent B, a C2 to C6 alkenyl group optionally substituted with a substituent A, a C2 to C6 haloalkenyl group, a C2 to C6 alkynyl group optionally substituted with a substituent A, a C2 to C6 haloalkynyl group, a C1 to C6 alkoxy group, a C1 to C6 haloalkoxy group, a C3 to C8 cycloalkoxy group, a phenyl group optionally substituted with 0 to 5 substituents B, or a 5- or 6-membered aromatic heterocyclic group optionally substituted with 0 to 4 substituents B, or Rx1 and Rx2 together with a nitrogen atom to which they are bonded form an aziridinyl group, an azetidinyl group, a pyrrolidinyl group, a piperidinyl group, a piperazinyl group, a morpholinyl group, a thiomorpholinyl group, a dioxothiomorpholinyl group, a thiazolidinyl group, an azepanyl group, or an azocanyl group) in R1 in Formula (1-1) has the same meaning as above.

The “Rx1Rx2NC(═O)—” is preferably a carbamoyl group, a methylcarbamoyl group, an ethylcarbamoyl group, a propylcarbamoyl group, an isopropylcarbamoyl group, a butylcarbamoyl group, an isobutylcarbamoyl group, a sec-butylcarbamoyl group, a t-butylcarbamoyl group, a pentylcarbamoyl group, an isopentylcarbamoyl group, a 2-methylbutylcarbamoyl group, a neopentylcarbamoyl group, a 1-ethylpropylcarbamoyl group, a hexylcarbamoyl group, a methoxymethylcarbamoyl group, an ethoxymethylcarbamoyl group, a propyloxymethylcarbamoyl group, an isopropyloxymethylcarbamoyl group, a 2-methoxyethylcarbamoyl group, a 2-ethoxyethylcarbamoyl group, a cyanomethylcarbamoyl group, a 1-cyanoethylcarbamoyl group, a 2-cyanoethylcarbamoyl group, a cyclopropylmethylcarbamoyl group, a cyclobutylmethylcarbamoyl group,
a benzylcarbamoyl group, a 1-phenethylcarbamoyl group, a 1,1-difluoroethylcarbamoyl group, a 2,2-difluoroethylcarbamoyl group, a 2,2,2-trifluoroethylcarbamoyl group, a 2,2,3,3,3-pentafluoropropylcarbamoyl group, a 2,2,3,3,4,4,4-heptafluorobutylcarbamoyl group, a 3,3,4,4,5,5,5-heptafluoropentylcarbamoyl group, a cyclopropylcarbamoyl group, a 1-cyanocyclopropylcarbamoyl group, an allylcarbamoyl group, a 3,3-difluoroallylcarbamoyl group, a propargylcarbamoyl group, a methoxycarbamoyl group, a trifluoroethoxycarbamoyl group, a phenylcarbamoyl group, a 4-cyanophenylcarbamoyl group, a 2-fluorophenylcarbamoyl group, a 3-fluorophenylcarbamoyl group, a 4-fluorophenylcarbamoyl group, a 2-chlorophenylcarbamoyl group, a 3-chlorophenylcarbamoyl group, a 4-chlorophenylcarbamoyl group, a 2-trifluoromethylphenylcarbamoyl group, a 3-trifluoromethylphenylcarbamoyl group, a 4-trifluoromethylphenylcarbamoyl group, a 2-methoxyphenylcarbamoyl group, a 3-methoxyphenylcarbamoyl group, a 4-methoxyphenylcarbamoyl group, a pyrrolidinecarbonyl group, a piperidinecarbonyl group, a piperazinecarbonyl group, a morpholinecarbonyl group, a thiomorpholinecarbonyl group, a dioxothiomorpholinecarbonyl group, a thiazolidinecarbonyl group, an azepanecarbonyl group, an azocancarbonyl group, a dimethylcarbamoyl group, an ethylmethylcarbamoyl group, a diethylcarbamoyl group, a methylpropylcarbamoyl group, an ethylpropylcarbamoyl group, an isopropylmethylcarbamoyl group, an isopropylethylcarbamoyl group, a diisopropylcarbamoyl group, a butylmethylcarbamoyl group, a butylethylcarbamoyl group, a sec-butylmethylcarbamoyl group, a sec-butylethylcarbamoyl group, an isobutylmethylcarbamoyl group, an isobutylethylcarbamoyl group,
a t-butylmethylcarbamoyl group, a t-butylethylcarbamoyl group, a methylpentylcarbamoyl group, an ethylpentylearbamoyl group, an isopentylmethylcarbamoyl group, an isopentylethylcarbamoyl group, a methyl(2-methylbutyl)carbamoyl group, an ethyl(2-methylbutyl)carbamoyl group, a hexylmethylcarbamoyl group, a hexylethylcarbamoyl group, a (methoxymethyl)methylcarbamoyl group, an (ethoxymethyl)methylcarbamoyl group, a (methoxyethyl)methylcarbamoyl group, an (ethoxyethyl)methylcarbamoyl group, a (propyloxymethyl)methylcarbamoyl group, an (isopropyloxymethyl)methylcarbamoyl group, a (methoxymethyl)ethylcarbamoyl group, an (ethoxymethyl)ethylcarbamoyl group, a (cyanomethyl)methylcarbamoyl group, a (cyanomethyl)ethylcarbamoyl group, a (cyclopropylmethyl)methylcarbamoyl group, a (cyclopropylmethyl)ethylcarbamoyl group, a (benzyl)methylcarbamoyl group, a methyl(pyridin-3-ylmethyl)carbamoyl group, a methyl(2,2,2-trifluoroethyl)carbamoyl group, a methyl(2,2,3,3,3-pentafluoropropyl)carbamoyl group, a methyl(2,2,3,3,4,4,4-heptafluorobutyl)carbamoyl group, a methyl(3,3,4,4,5,5,5-heptafluoropentyl)carbamoyl group, a (cyclopropyl)methylcarbamoyl group, a (cyclopropyl)ethylcarbamoyl group, a (1-cyanocyclopropyl)methylcarbamoyl group, a (1-cyanocyclopropyl)ethylcarbamoyl group, a (cyclobutyl)methylcarbamoyl group, a (cyclopentyl)methylcarbamoyl group, a (cyclohexyl)methylcarbamoyl group, an (allyl)methylcarbamoyl group, a methyl(2-propyn-1-yl)carbamoyl group, an ethyl(2-propyn-1-yl)carbamoyl group, a (methoxy) methylcarbamoyl group, an (ethoxy)methylcarbamoyl group, a (trifluoroethoxy)methylcarbamoyl group, a methylphenylcarbamoyl group, a diphenylcarbamoyl group, a methyl(pyridin-2-yl)carbamoyl group,
a methyl(6-methyl-pyridin-2-yl)carbamoyl group, a methyl(pyridin-3-yl)carbamoyl group, or a methyl(pyridin-4-yl)carbamoyl group,
more preferably a methylcarbonyl group, an ethylcarbamoyl group, a pyrrolidinecarbonyl group, a piperidinecarbonyl group, a piperazinecarbonyl group, a morpholinecarbonyl group, a thiomorpholinecarbonyl group, a dioxothiomorpholinecarbonyl group, a thiazolidinecarbonyl group, an azepanecarbonyl group, an azocancarbonyl group, a dimethylcarbamoyl group, an ethylmethylcarbamoyl group, a diethylcarbamoyl group, a methylpropylcarbamoyl group, an ethylpropylcarbamoyl group, an isopropylmethylcarbamoyl group, an isopropylethylcarbamoyl group, a diisopropylcarbamoyl group, a butylmethylcarbamoyl group, a butylethylcarbamoyl group, a sec-butylmethylcarbamoyl group, a see-butylethylcarbamoyl group, an isobutylmethylcarbamoyl group, an isobutylethylcarbamoyl group, a t-butylmethylcarbamoyl group, a t-butylethylcarbamoyl group, a methylpentylcarbamoyl group, an ethylpentylcarbamoyl group, an isopentylmethylcarbamoyl group, an isopentylethylcarbamoyl group, a methyl(2-methylbutyl)carbamoyl group, an ethyl(2-methylbutyl)carbamoyl group, a hexylmethylcarbamoyl group, a hexylethylcarbamoyl group, a (methoxymethyl)methylcarbamoyl group, an (ethoxymethyl)methylcarbamoyl group, a (methoxyethyl)methylcarbamoyl group, an (ethoxyethyl)methylcarbamoyl group, a (propyloxymethyl)methylcarbamoyl group, an (isopropyloxymethyl)methylcarbamoyl group, a (methoxymethyl)ethylcarbamoyl group, an (ethoxymethyl)ethylcarbamoyl group, a (cyanomethyl)methylcarbamoyl group, a (cyanomethyl)ethylcarbamoyl group, a (cyclopropylmethyl)methylcarbamoyl group, a (cyclopropylmethyl)ethylcarbamoyl group, a (benzyl)methylcarbamoyl group, a methyl(pyridin-3-ylmethyl)carbamoyl group, a methyl(2,2,2-trifluoroethyl)carbamoyl group, a methyl(2,2,3,3,3-pentafluoropropyl)carbamoyl group, a methyl(2,2,3,3,4,4,4-heptafluorobutyl)carbamoyl group, a methyl(3,3,4,4,5,5,5-heptafluoropentyl)carbamoyl group, a (cyclopropyl)methylcarbamoyl group, a (cyclopropyl)ethylcarbamoyl group, a (1-cyanocyclopropyl)methylcarbamoyl group, a (1-cyanocyclopropyl)ethylcarbamoyl group, a (cyclobutyl)methylcarbamoyl group, a (cyclopentyl)methylcarbamoyl group, a (cyclohexyl)methylcarbamoyl group, an (allyl)methylcarbamoyl group, a methyl(2-propyn-1-yl)carbamoyl group, an ethyl(2-propyn-1-yl)carbamoyl group, a (methoxy)methylcarbamoyl group, an (ethoxy)methylcarbamoyl group, a (trifluoroethoxy)methylcarbamoyl group, a methylphenylcarbamoyl group, a diphenylcarbamoyl group, a methyl(pyridin-2-yl)carbamoyl group, a methyl(6-methyl-pyridin-2-yl)carbamoyl group, a methyl(pyridin-3-yl)carbamoyl group, or a methyl(pyridin-4-yl)carbamoyl group,
still more preferably an ethylcarbamoyl group, a pyrrolidinecarbonyl group, a piperidinecarbonyl group, a morpholinecarbonyl group, a thiomorpholinecarbonyl group, a dioxothiomorpholinecarbonyl group, a thiazolidinecarbonyl group, an azepanecarbonyl group, an azocancarbonyl group, a dimethylcarbamoyl group, an ethylmethylcarbamoyl group, a diethylcarbamoyl group, a methylpropylcarbamoyl group, an isopropylmethylcarbamoyl group, a diisopropylcarbamoyl group, a butylmethylcarbamoyl group, a butylethylcarbamoyl group, a sec-butylmethylcarbamoyl group, a sec-butylethylcarbamoyl group, an isobutylmethylcarbamoyl group, an isobutylethylcarbamoyl group, a t-butylmethylcarbamoyl group, a (methoxyethyl)methylcarbamoyl group, a (cyanomethyl)methylcarbamoyl group, a (cyanomethyl)ethylcarbamoyl group, a (cyclopropylmethyl)methylcarbamoyl group, a (cyclo propylmethyl)ethylcarbamoyl group, a (benzyl)methylcarbamoyl group, a methyl(pyridin-3-ylmethyl)carbamoyl group, a methyl(2,2,2-trifluoroethyl)carbamoyl group, a (cyclopropyl)methylcarbamoyl group, a (cyclopentyl)methylcarbamoyl group, a (cyclohexyl)methylcarbamoyl group, an (allyl)methylcarbamoyl group, a methyl(2-propyn-1-yl)carbamoyl group, an ethyl(2-propyn-1-yl)carbamoyl group, a (methoxy)methylcarbamoyl group, an (ethoxy)methylcarbamoyl group, a (trifluoroethoxy)methylcarbamoyl group, a methyl phenylcarbamoyl group, a diphenylcarbamoyl group, a methyl(pyridin-2-yl)carbamoyl group, a methyl(6-methyl-pyridin-2-yl)carbamoyl group, a methyl(pyridin-3-yl)carbamoyl group, or a methyl(pyridin-4-yl)carbamoyl group, and
particularly preferably an ethylcarbamoyl group, a pyrrolidinecarbonyl group, a piperidinecarbonyl group, a morpholinecarbonyl group, a thiomorpholinecarbonyl group, a thiazolidinecarbonyl group, an azepanecarbonyl group, an azocancarbonyl group, a dimethylcarbamoyl group, an ethylmethylcarbamoyl group, a diethylcarbamoyl group, a methylpropylcarbamoyl group, an isopropylmethylcarbamoyl group, a diisopropylcarbamoyl group, a butylmethylcarbamoyl group, an isobutylmethylcarbamoyl group, a t-butylmethylcarbamoyl group, a (methoxyethyl)methylcarbamoyl group, a (cyanomethyl)methylcarbamoyl group, a (cyclopropylmethyl)methylcarbamoyl group, a methyl(pyridin-3-ylmethyl)carbamoyl group, a methyl(2,2,2-trifluoroethyl)carbamoyl group, a (cyclopropyl)methylcarbamoyl group, a methyl(2-propyn-1-yl)carbamoyl group, a (methoxy)methylcarbamoyl group, a methylphenylcarbamoyl group, a diphenylcarbamoyl group, a methyl(pyridin-2-yl)carbamoyl group, a methyl(6-methyl-pyridin-2-yl)carbamoyl group, a methyl(pyridin-3-yl)carbamoyl group, or a methyl(pyridin-4-yl)carbamoyl group.

Each term of “Rx3C(═O)—” (where Rx3 represents a hydrogen atom, a hydroxyl group, a halogen atom, a C1 to C6 alkyl group optionally substituted with a substituent A, a C1 to C6 haloalkyl group, a C3 to C8 cycloalkyl group optionally substituted with a substituent B, a C2 to C6 alkenyl group optionally substituted with a substituent A, a C2 to C6 haloalkenyl group, a C2 to C6 alkynyl group optionally substituted with a substituent A, a C2 to C6 haloalkynyl group, a phenyl group optionally substituted with 0 to 5 substituents B, or a 5- or 6-membered aromatic heterocyclic group optionally substituted with 0 to 4 substituents B) in R1 in Formula (1-1) has the same meaning as above.

The “Rx3C(═O)—” is preferably an acetyl group, a 1-cyanoacetyl group, a 1-methoxyacetyl group, a propionyl group, an isobutyryl group, a difluoroacetyl group, a trifluoroacetyl group, a benzoyl group, a 4-fluorobenzoyl group, or a 4-trifluorobenzoyl group, more preferably an acetyl group, a propionyl group, a difluoroacetyl group, a trifluoroacetyl group, or a benzoyl group, and particularly preferably an acetyl group, a trifluoroacetyl group, or a benzoyl group.

Each term of “Rx3OC(═O)—” (where Rx3 represents a hydrogen atom, a hydroxyl group, a halogen atom, a C1 to C6 alkyl group optionally substituted with a substituent A, a C1 to C6 haloalkyl group, a C3 to C8 cycloalkyl group optionally substituted with a substituent B, a C2 to C6 alkenyl group optionally substituted with a substituent A, a C2 to C6 haloalkenyl group, a C2 to C6 alkynyl group optionally substituted with a substituent A, a C2 to C6 haloalkynyl group, a phenyl group optionally substituted with 0 to 5 substituents B, or a 5- or 6-membered aromatic heterocyclic group optionally substituted with 0 to 4 substituents B) in R1 in Formula (1-1) has the same meaning as above.

The “Rx3OC(═O)—” is preferably a methoxycarbonyl group, an ethoxycarbonyl group, an isopropyloxycarbonyl group, a difluoromethoxycarbonyl group, a trifluoromethoxycarbonyl group, a cyclopropyloxycarbonyl group, a cyclopentyloxycarbonyl group, an allyloxycarbonyl group, a propargyloxycarbonyl group, a phenyloxycarbonyl group, or a 4-nitrophenyloxycarbonyl group, more preferably a methoxycarbonyl group, an ethoxycarbonyl group, an isopropyloxycarbonyl group, a cyclopropyloxycarbonyl group, a propargyloxycarbonyl group, a phenyloxycarbonyl group, or a 4-nitrophenyloxycarbonyl group, and particularly preferably a methoxycarbonyl group or an ethoxycarbonyl group.

Each term of “Rx4S(O)p-” (where Rx4 represents a hydroxyl group, a halogen atom, a C1 to C6 alkyl group optionally substituted with a substituent A, a C1 to C6 haloalkyl group, a C3 to C8 cycloalkyl group optionally substituted with a substituent B, a C2 to C6 alkenyl group optionally substituted with a substituent A, a C2 to C6 haloalkenyl group, a C2 to C6 alkynyl group optionally substituted with a substituent A, a C2 to C6 haloalkynyl group, or a phenyl group optionally substituted with 0 to 5 substituents B, and p represents an integer of 0, 1, or 2) in R1 in Formula (1-1) has the same meaning as above.

The “Rx4S(O)p-” is preferably a methylsulfonyl group, an ethylsulfonyl group, a trifluoromethylsulfonyl group, a 1,1,2,2,2-pentafluoroethylsulfonyl group, a cyclopropylsulfonyl group, a cyclopentylsulfonyl group, a phenylsulfonyl group, a 4-fluorophenylsulfonyl group, a 4-chlorophenylsulfonyl group, a 4-methylphenylsulfonyl group, or a 4-trifluoromethylphenylsulfonyl group, more preferably a methylsulfonyl group, an ethylsulfonyl group, a trifluoromethylsulfonyl group, a 4-methylphenylsulfonyl group, or a 4-trifluoromethylphenylsulfonyl group, and particularly preferably a methylsulfonyl group, an ethylsulfonyl group, or a 4-methylphenylsulfonyl group.

The term “3- to 6-membered ring group containing one or two oxygen atoms” in R1 in Formula (1-1) has the same meaning as above. The “3- to 6-membered ring group containing one or two oxygen atoms” is preferably an oxiran-2-yl group, an oxetan-3-yl group, a tetrahydrofuran-2-yl group, a tetrahydrofuran-3-yl group, a tetrahydro-2H-pyran-2-yl group, a tetrahydro-2H-pyran-3-yl group, a tetrahydro-2H-pyran-4-yl group, a 1,4-dioxan-2-yl group, a 3-dihydrofuran-2(3H)-one group, a 4-dihydrofuran-2(3H)-one group, a 3-tetrahydro-2H-pyran-2-one group, or a 4-tetrahydro-2H-pyran-2-one group, more preferably a tetrahydrofuran-2-yl group, a tetrahydrofiran-3-yl group, a tetrahydro-2H-pyran-2-yl group, a tetrahydro-2H-pyran-3-yl group, a tetrahydro-2H-pyran-4-yl group, a 3-dihydro Furan-2(3H)-one group, or a 4-dihydrofuran-2(3H)-one group, still more preferably a tetrahydro-2H-pyran-2-yl group, a tetrahydro-2H-pyran-4-yl group, or a 3-dihydrofuran-2(3H)-one group, and particularly preferably a tetrahydro-2H-pyran-4-yl group or a 3-dihydrofuran-2(3H)-one group.

In Formula (1-2), R2 represents a hydrogen atom, a C1 to C6 alkyl group optionally substituted with a substituent A, a C1 to C6 haloalkyl group, a C3 to C8 cycloalkyl group optionally substituted with a substituent B, a C2 to C6 alkenyl group optionally substituted with a substituent A, a C2 to C6 haloalkenyl group, a C2 to C6 alkynyl group optionally substituted with a substituent A, a C2 to C6 haloalkynyl group, a phenyl group optionally substituted with 0 to 5 substituents B, a 5- or 6-membered aromatic heterocyclic group optionally substituted with 0 to 4 substituents B, Rx1Rx2NC(═O)— (where Rx1 and Rx2 each independently represent a hydrogen atom, a hydroxyl group, a cyano group, a C1 to C6 alkyl group optionally substituted with a substituent A, a C1 to C6 haloalkyl group, a C3 to C8 cycloalkyl group optionally substituted with a substituent B, a C2 to C6 alkenyl group optionally substituted with a substituent A, a C2 to C6 haloalkenyl group, a C2 to C6 alkynyl group optionally substituted with a substituent A, a C2 to C6 haloalkynyl group, a C1 to C6 alkoxy group, a C1 to C6 haloalkoxy group, a C3 to C8 cycloalkoxy group, a phenyl group optionally substituted with 0 to 5 substituents B, or a 5- or 6-membered aromatic heterocyclic group optionally substituted with 0 to 4 substituents B, or Rx1 and Rx2 together with a nitrogen atom to which they are bonded form an aziridinyl group, an azetidinyl group, a pyrrolidinyl group, a piperidinyl group, a piperazinyl group, a morpholinyl group, a thiomorpholinyl group, a dioxothiomorpholinyl group, a thiazolidinyl group, an azepanyl group, or an azocanyl group), Rx3C(═O)— (where Rx3 represents a hydrogen atom, a hydroxyl group, a halogen atom, a C1 to C6 alkyl group optionally substituted with a substituent A, a C1 to C6 haloalkyl group, a C3 to C8 cycloalkyl group optionally substituted with a substituent B, a C2 to C6 alkenyl group optionally substituted with a substituent A, a C2 to C6 haloalkenyl group, a C2 to C6 alkynyl group optionally substituted with a substituent A, a C2 to C6 haloalkynyl group, a phenyl group optionally substituted with 0 to 5 substituents B, or a 5- or 6-membered aromatic heterocyclic group optionally substituted with 0 to 4 substituents B), Rx3OC(═O)— (where Rx3 has the same meaning as above), Rx4S(O)p- (where Rx4 represents a hydroxyl group, a halogen atom, a C1 to C6 alkyl group optionally substituted with a substituent A, a C1 to C6 haloalkyl group, a C3 to C8 cycloalkyl group optionally substituted with a substituent B, a C2 to C6 alkenyl group optionally substituted with a substituent A, a C2 to C6 haloalkenyl group, a C2 to C6 alkynyl group optionally substituted with a substituent A, a C2 to C6 haloalkynyl group, or a phenyl group optionally substituted with 0 to 5 substituents B, and p represents an integer of 0, 1, or 2), or a 3- to 6-membered ring group containing one or two oxygen atoms.

Among them, R2 is preferably a hydrogen atom, a C1 to C6 alkyl group optionally substituted with a substituent A, a C1 to C6 haloalkyl group, a C3 to C8 cycloalkyl group optionally substituted with a substituent B, a C2 to C6 alkenyl group optionally substituted with a substituent A, a C2 to C6 alkynyl group optionally substituted with a substituent A, or Rx1Rx2NC(═O)— (where Rx1 and Rx2 have the same meanings as above).

Further, R2 is preferably a hydrogen atom, a C1 to C6 alkyl group optionally substituted with a substituent A, a C1 to C6 haloalkyl group, a C3 to C8 cycloalkyl group optionally substituted with a substituent B, or Rx1Rx2NC(═O)— (where Rx1 and Rx2 have the same meanings as above), and

R2 is particularly preferably a C1 to C6 alkyl group optionally substituted with a substituent A, a C1 to C6 haloalkyl group, or a C3 to C8 cycloalkyl group optionally substituted with a substituent B.

R2 in Formula (1-2) contains a hydrogen atom.

The C1 to C6 alkyl group of the “C1 to C6 alkyl group optionally substituted with a substituent A” in R2 in Formula (1-2) has the same meaning as above, and is preferably a methyl group, an ethyl group, a propyl group, an isopropyl group, a butyl group, an isobutyl group, a sec-butyl group, a pentyl group, or a hexyl group, more preferably a methyl group, an ethyl group, a propyl group, an isopropyl group, a butyl group, an isobutyl group, a sec-butyl group, or a pentyl group, and particularly preferably a methyl group, an ethyl group, a propyl group, an isopropyl group, a butyl group, an isobutyl group, or a sec-butyl group. When having the substituent described in the substituent A, a hydrogen atom in the C1 to C6 alkyl group is optionally substituted with the substituent described in the substituent A.

The “C1 to C6 alkyl group optionally substituted with a substituent A” is preferably a methyl group, an ethyl group, a propyl group, an isopropyl group, a butyl group, an isobutyl group, a sec-butyl group, a pentyl group, a hexyl group, a cyanomethyl group, a 1-cyanoethyl group, a 2-cyanoethyl group, a 3-cyanopropyl group, a 2-cyanopropan-2-yl group, a cyclopropylmethyl group, a cyclobutylmethyl group, a methoxymethyl group, an ethoxymethyl group, a methoxyethyl group, an ethoxyethyl group, an isopropyloxymethyl group, an isopropyloxyethyl group,
a benzyl group, a 1-phenethyl group, a 2-phenethyl group, a 4-cyanophenylmethyl group, a 2-fluorophenylmethyl group, a 3-fluorophenylmethyl group, a 4-fluorophenylmethyl group, a 2-chlorophenylmethyl group, a 3-chlorophenylmethyl group, a 3,5-dichlorophenylmethyl group, a 4-chlorophenylmethyl group, a 2-methylphenylmethyl group, a 3-methylphenylmethyl group, a 4-methylphenylmethyl group, a 2-trifluoromethylphenylmethyl group, a 3-trifluoromethylphenylmethyl group, a 4-trifluoromethylphenylmethyl group, a 2-methoxyphenylmethyl group, a 3-methoxyphenylmethyl group, a 4-methoxyphenylmethyl group, a 4-phenoxyphenylmethyl group, a 4-methanesulfonylphenylmethyl group, a trifluoromethylsulfanylphenylmethyl group,
a pyridin-2-ylmethyl group, a pyridin-2-ylmethyl-N-oxide group, a 3-fluoropyridin-2-ylmethyl group, a 4-fluoropyridin-2-ylmethyl group, a 5-fluoropyridin-2-ylmethyl group, a 6-fluoropyridin-2-ylmethyl group, a 3-chloropyridin-2-ylmethyl group, a 4-chloropyridin-2-ylmethyl group, a 5-chloropyridin-2-ylmethyl group, a 6-chloropyridin-2-ylmethyl group, a 3-bromopyridin-2-ylmethyl group, a 4-bromopyridin-2-ylmethyl group, a 5-bromopyridin-2-ylmethyl group, a 6-bromopyridin-2-ylmethyl group, a 3-methylpyridin-2-ylmethyl group, a 4-methylpyridin-2-ylmethyl group, a 5-methylpyridin-2-ylmethyl group, a 6-methylpyridin-2-ylmethyl group, a 3-trifluoromethylpyridin-2-ylmethyl group, a 4-trifluoromethylpyridin-2-ylmethyl group, a 5-trifluoromethylpyridin-2-ylmethyl group, a 6-trifluoromethylpyridin-2-ylmethyl group, a 3-cyclopropylpyridin-2-ylmethyl group, a 4-cyclopropylpyridin-2-ylmethyl group, a 5-cyclopropylpyridin-2-ylmethyl group, a 6-cyclopropylpyridin-2-ylmethyl group, a 3-methoxypyridin-2-ylmethyl group, a 4-methoxypyridin-2-ylmethyl group, a 5-methoxypyridin-2-ylmethyl group, a 6-methoxypyridin-2-ylmethyl group,
a pyridin-3-ylmethyl group, a 2-fluoropyridin-3-ylmethyl group, a 4-fluoropyridin-3-ylmethyl group, a 5-fluoropyridin-3-ylmethyl group, a 6-fluoropyridin-3-ylmethyl group, a 2-chloropyridin-3-ylmethyl group, a 4-chloropyridin-3-ylmethyl group, a 5-chloropyridin-3-ylmethyl group, a 6-chloropyridin-3-ylmethyl group, a 2-bromopyridin-3-ylmethyl group, a 4-bromopyridin-3-ylmethyl group, a 5-bromopyridin-3-ylmethyl group, a 6-bromopyridin-3-ylmethyl group, a 2-methylpyridin-3-ylmethyl group, a 4-methylpyridin-3-ylmethyl group, a 5-methylpyridin-3-ylmethyl group, a 6-methylpyridin-3-ylmethyl group, a 2-trifluoromethylpyridin-3-ylmethyl group, a 4-trifluoromethylpyridin-3-ylmethyl group, a 5-trifluoromethylpyridin-3-ylmethyl group, a 6-trifluoromethylpyridin-3-ylmethyl group, a 2-methoxypyridin-3-ylmethyl group, a 4-methoxypyridin-3-ylmethyl group, a 5-methoxypyridin-3-ylmethyl group, a 6-methoxypyridin-3-ylmethyl group,
a pyridin-4-ylmethyl group, a 2-fluoropyridin-4-ylmethyl group, a 3-fluoropyridin-4-ylmethyl group, a 2-chloropyridin-4-ylmethyl group, a 3-chloropyridin-4-ylmethyl group, a 2-bromopyridin-4-ylmethyl group, a 3-bromopyridin-4-ylmethyl group, a 2-methylpyridin-4-ylmethyl group, a 3-methylpyridin-4-ylmethyl group, a 2-trifluoromethylpyridin-4-ylmethyl group, a 3-trifluoromethylpyridin-4-ylmethyl group, a 2-methoxypyridin-4-ylmethyl group, a 3-methoxypyridin-4-ylmethyl group,
a pyridazin-3-ylmethyl group, a 4-fluoropyridazin-3-ylmethyl group, a 5-fluoropyridazin-3-ylmethyl group, a 6-fluoropyridazin-3-ylmethyl group, a 4-chloropyridazin-3-ylmethyl group, a 5-chloropyridazin-3-ylmethyl group, a 6-chloropyridazin-3-ylmethyl group, a 4-bromopyridazin-3-ylmethyl group, a 5-bromopyridazin-3-ylmethyl group, a 6-bromopyridazin-3-ylmethyl group, a 4-methylpyridazin-3-ylmethyl group, a 5-methylpyridazin-3-ylmethyl group, a 6-methylpyridazin-3-ylmethyl group, a 4-trifluoromethylpyridazin-3-ylmethyl group, a 5-trifluoromethylpyridazin-3-ylmethyl group, a 6-trifluoromethylpyridazin-3-ylmethyl group, a 4-methoxypyridazin-3-ylmethyl group, a 5-methoxypyridazin-3-ylmethyl group, a 6-methoxypyridazin-3-ylmethyl group,
a pyridazin-4-ylmethyl group, a 3-fluoropyridazin-4-ylmethyl group, a 5-fluoropyridazin-4-ylmethyl group, a 6-fluoropyridazin-4-ylmethyl group, a 3-chloropyridazin-4-ylmethyl group, a 5-chloropyridazin-4-ylmethyl group, a 6-chloropyridazin-4-ylmethyl group, a 3-bromopyridazin-4-ylmethyl group, a 5-bromopyridazin-4-ylmethyl group, a 6-bromopyridazin-4-ylmethyl group, a 3-methylpyridazin-4-ylmethyl group, a 5-methylpyridazin-4-ylmethyl group, a 6-methylpyridazin-4-ylmethyl group, a 3-trifluoromethylpyridazin-4-ylmethyl group, a 5-trifluoromethylpyridazin-4-ylmethyl group, a 6-trifluoromethylpyridazin-4-ylmethyl group, a 3-methoxypyridazin-4-ylmethyl group, a 5-methoxypyridazin-4-ylmethyl group, a 6-methoxypyridazin-4-ylmethyl group,
a pyrimidin-2-ylmethyl group, a 4-fluoropyrimidin-2-ylmethyl group, a 5-fluoropyrimidin-2-ylmethyl group, a 4-chloropyrimidin-2-ylmethyl group, a 5-chloropyrimidin-2-ylmethyl group, a 4-bromopyrimidin-2-ylmethyl group, a 5-bromopyrimidin-2-ylmethyl group, a 4-methylpyrimidin-2-ylmethyl group, a 5-methylpyrimidin-2-ylmethyl group, a 4-trifluoromethylpyrimidin-2-ylmethyl group, a 5-trifluoromethylpyrimidin-2-ylmethyl group, a 4-methoxypyrimidin-2-ylmethyl group, a 5-methoxypyrimidin-2-ylmethyl group,
a pyrimidin-4-ylmethyl group, a 2-fluoropyrimidin-4-ylmethyl group, a 5-fluoropyrimidin-4-ylmethyl group, a 6-fluoropyrimidin-4-ylmethyl group, a 2-chloropyrimidin-4-ylmethyl group, a 5-chloropyrimidin-4-ylmethyl group, a 6-chloropyrimidin-4-ylmethyl group, a 2-bromopyrimidin-4-ylmethyl group, a 5-bromopyrimidin-4-ylmethyl group, a 6-bromopyrimidin-4-ylmethyl group, a 2-methylpyrimidin-4-ylmethyl group, a 5-methylpyrimidin-4-ylmethyl group, a 6-methylpyrimidin-4-ylmethyl group, a 2-trifluoromethylpyrimidin-4-ylmethyl group, a 5-trifluoromethylpyrimidin-4-ylmethyl group, a 6-trifluoromethylpyrimidin-4-ylmethyl group, a 2-methoxypyrimidin-4-ylmethyl group, a 5-methoxypyrimidin-4-ylmethyl group, a 6-methoxypyrimidin-4-ylmethyl group,
a pyrimidin-5-ylmethyl group, a 2-fluoropyrimidin-5-ylmethyl group, a 4-fluoropyrimidin-5-ylmethyl group, a 6-fluoropyrimidin-5-ylmethyl group, a 2-chloropyrimidin-5-ylmethyl group, a 4-chloropyrimidin-5-ylmethyl group, a 6-chloropyrimidin-5-ylmethyl group, a 2-bromopyrimidin-5-ylmethyl group, a 4-bromopyrimidin-5-ylmethyl group, a 6-bromopyrimidin-5-ylmethyl group, a 2-methylpyrimidin-5-ylmethyl group, a 4-methylpyrimidin-5-ylmethyl group, a 6-methylpyrimidin-5-ylmethyl group, a 2-trifluoromethylpyrimidin-5-ylmethyl group, a 4-trifluoromethylpyrimidin-5-ylmethyl group, a 6-trifluoromethylpyrimidin-5-ylmethyl group, a 2-methoxypyrimidin-5-ylmethyl group, a 4-methoxypyrimidin-5-ylmethyl group, a 6-methoxypyrimidin-5-ylmethyl group,
a pyrazin-2-ylmethyl group, a 3-fluoropyrazin-2-ylmethyl group, a 5-fluoropyrazin-2-ylmethyl group, a 6-fluoropyrazin-2-ylmethyl group, a 3-chloropyrazin-2-ylmethyl group, a 5-chloropyrazin-2-ylmethyl group, a 6-chloropyrazin-2-ylmethyl group, a 3-bromopyrazin-2-ylmethyl group, a 5-bromopyrazin-2-ylmethyl group, a 6-bromopyrazin-2-ylmethyl group, a 3-methylpyrazin-2-ylmethyl group, a 5-methylpyrazin-2-ylmethyl group, a 6-methylpyrazin-2-ylmethyl group, a 3-trifluoromethylpyrazin-2-ylmethyl group, a 5-trifluoromethylpyrazin-2-ylmethyl group, a 6-trifluoromethylpyrazin-2-ylmethyl group, a 3-methoxypyrazin-2-ylmethyl group, a 5-methoxypyrazin-2-ylmethyl group, a 6-methoxypyrazin-2-ylmethyl group,
a 1-(pyridin-2-yl)ethyl group, a 1-(3-fluoropyridin-2-yl)ethyl group, a 1-(4-fluoropyridin-2-yl)ethyl group, a 1-(5-fluoropyridin-2-yl)ethyl group, a 1-(6-fluoropyridin-2-yl)ethyl group, a 1-(3-chloropyridin-2-yl)ethyl group, a 1-(4-chloropyridin-2-yl)ethyl group, a 1-(5-chloropyridin-2-yl)ethyl group, a 1-(6-chloropyridin-2-yl)ethyl group, a 1-(3-bromopyridin-2-yl)ethyl group, a 1-(4-bromopyridin-2-yl)ethyl group, a 1-(5-bromopyridin-2-yl)ethyl group, a 1-(6-bromopyridin-2-yl) yl)ethyl group, a 1-(3-methylpyridin-2-yl)ethyl group, a 1-(4-methylpyridin-2-yl)ethyl group, a 1-(5-methylpyridin-2-yl)ethyl group, a 1-(6-methylpyridin-2-yl)ethyl group, a 1-(3-trifluoromethylpyridin-2-yl)ethyl group, a 1-(4-trifluoromethylpyridin-2-yl)ethyl group, a 1-(5-trifluoromethylpyridin-2-yl)ethyl group, a 1-(6-trifluoromethylpyridin-2-yl)ethyl group, a 1-(3-methoxypyridin-2-yl)ethyl group, a 1-(4-methoxypyridin-2-yl)ethyl group, a 1-(5-methoxypyridin-2-yl)ethyl group, a 1-(6-methoxypyridin-2-yl)ethyl group,
a 1-(pyridin-3-yl)ethyl group, a 1-(2-fluoropyridin-3-yl)ethyl group, a 1-(4-fluoropyridin-3-yl)ethyl group, a 1-(5-fluoropyridin-3-yl)ethyl group, a 1-(6-fluoropyridin-3-yl)ethyl group, a 1-(2-chloropyridin-3-yl)ethyl group, a 1-(4-chloropyridin-3-yl)ethyl group, a 1-(5-chloropyridin-3-yl)ethyl group, a 1-(6-chloropyridin-3-yl)ethyl group, a 1-(2-bromopyridin-3-yl)ethyl group, a 1-(4-bromopyridin-3-yl)ethyl group, a 1-(5-bromopyridin-3-yl)ethyl group, a 1-(6-bromopyridin-3-yl)ethyl group, a 1-(2-methylpyridin-3-yl)ethyl group, a 1-(4-methylpyridin-3-yl)ethyl group, a 1-(5-methylpyridin-3-yl)ethyl group, a 1-(6-methylpyridin-3-yl)ethyl group, a 1-(2-trifluoromethylpyridin-3-yl)ethyl group, a 1-(4-trifluoromethylpyridin-3-yl)ethyl group, a 1-(5-trifluoromethylpyridin-3-yl)ethyl group, a 1-(6-trifluoromethylpyridin-3-yl)ethyl group, a 1-(2-methoxypyridin-3-yl)ethyl group, a 1-(4-methoxypyridin-3-yl)ethyl group, a 1-(5-methoxypyridin-3-yl)ethyl group, a 1-(6-methoxypyridin-3-yl)ethyl group,
a 1-(pyridin-4-yl)ethyl group, a 1-(2-fluoropyridin-4-yl)ethyl group, a 1-(3-fluoropyridin-4-yl)ethyl group, a 1-(2-chloropyridin-4-yl)ethyl group, a 1-(3-chloropyridin-4-yl)ethyl group, a 1-(2-bromopyridin-4-yl)ethyl group, a 1-(3-bromopyridin-4-yl)ethyl group, a 1-(2-methylpyridin-4-yl)ethyl group, a 1-(3-methylpyridin-4-yl)ethyl group, a 1-(2-trifluoromethylpyridin-4-yl)ethyl group, a 1-(3-trifluoromethylpyridin-4-yl)ethyl group, a 1-(2-methoxypyridin-4-yl)ethyl group, a 1-(3-methoxypyridin-4-yl)ethyl group,
a 1-(pyridazin-3-yl)ethyl group, a 1-(4-fluoropyridazin-3-yl)ethyl group, a 1-(5-fluoropyridazin-3-yl)ethyl group, a 1-(6-fluoropyridazin-3-yl)ethyl group, a 1-(4-chloropyridazin-3-yl)methyl group, a 1-(5-chloropyridazin-3-yl)ethyl group, a 1-(6-chloropyridazin-3-yl)ethyl group, a 1-(4-bromopyridazin-3-yl)ethyl group, a 1-(5-bromopyridazin-3-yl)ethyl group, a 1-(6-bromopyridazin-3-yl)ethyl group, a 1-(4-methylpyridazin-3-yl)ethyl group, a 1-(5-methylpyridazin-3-yl)ethyl group, a 1-(6-methylpyridazin-3-yl)ethyl group, a 1-(4-trifluoromethylpyridazin-3-yl)ethyl group, a 1-(5-trifluoromethylpyridazin-3-yl)ethyl group, a 1-(6-trifluoromethylpyridazin-3-yl)ethyl group, a 1-(4-methoxypyridazin-3-yl)ethyl group, a 1-(5-methoxypyridazin-3-yl)ethyl group, a 1-(6-methoxypyridazin-3-yl)ethyl group,
a 1-(pyridazin-4-yl)ethyl group, a 1-(3-fluoropyridazin-4-yl)ethyl group, a 1-(5-fluoropyridazin-4-yl)ethyl group, a 1-(6-fluoropyridazine-4-yl)ethyl group, a 1-(3-chloropyridazin-4-yl)ethyl group, a 1-(5-chloropyridazin-4-yl)ethyl group, a 1-(6-chloropyridazin-4-yl)ethyl group, a 1-(3-bromopyridazin-4-yl)ethyl group, a 1-(5-bromopyridazin-4-yl)ethyl group, a 1-(6-bromopyridazin-4-yl)ethyl group, a 1-(3-methylpyridazin-4-yl)ethyl group, a 1-(5-methylpyridazin-4-yl)ethyl group, a 1-(6-methylpyridazin-4-yl)ethyl group, a 1-(3-trifluoromethylpyridazin-4-yl)ethyl group, a 1-(5-trifluoromethylpyridazin-4-yl)ethyl group, a 1-(6-trifluoromethylpyridazin-4-yl)ethyl group, a 1-(3-methoxypyridazin-4-yl)ethyl group, a 1-(5-methoxypyridazin-4-yl)ethyl group, a 1-(6-methoxypyridazin-4-yl)ethyl group,
a 1-(pyrimidin-2-yl)ethyl group, a 1-(4-fluoropyrimidin-2-yl)ethyl group, a 1-(5-fluoropyrimidin-2-yl)ethyl group, a 1-(4-chloropyrimidin-2-yl)ethyl group, a 1-(5-chloropyrimidin-2-yl)ethyl group, a 1-(4-bromopyrimidin-2-yl)ethyl group, a 1-(5-bromopyrimidin-2-yl)ethyl group, a 1-(4-methylpyrimidin-2-yl)ethyl group, a 1-(5-methylpyrimidin-2-yl)ethyl group, a 1-(4-trifluoromethylpyrimidin-2-yl)ethyl group, a 1-(5-trifluoromethylpyrimidin-2-yl)ethyl group, a 1-(4-methoxypyrimidin-2-yl)ethyl group, a 1-(5-methoxypyrimidin-2-yl)ethyl group,
a 1-(pyrimidin-4-yl)ethyl group, a 1-(2-fluoropyrimidin-4-yl)ethyl group, a 1-(5-fluoropyrimidin-4-yl)ethyl group, a 1-(6-fluoropyrimidine-4-yl)ethyl group, a 1-(2-chloropyrimidin-4-yl)ethyl group, a 1-(5-chloropyrimidin-4-yl)ethyl group, a 1-(6-chloropyrimidin-4-yl)ethyl group, a 1-(2-bromopyrimidin-4-yl)ethyl group, a 1-(5-bromopyrimidin-4-yl)ethyl group, a 1-(6-bromopyrimidin-4-yl)ethyl group, a 1-(2-methylpyrimidin-4-yl)ethyl group, a 1-(5-methylpyrimidin-4-yl)ethyl group, a 1-(6-methylpyrimidin-4-yl)ethyl group, a 1-(2-trifluoromethylpyrimidin-4-yl)ethyl group, a 1-(5-trifluoromethylpyrimidin-4-yl)ethyl group, a 1-(6-trifluoromethylpyrimidin-4-yl)ethyl group, a 1-(2-methoxypyrimidin-4-yl)ethyl group, a 1-(5-methoxypyrimidin-4-yl)ethyl group, a 1-(6-methoxypyrimidin-4-yl)ethyl group,
a 1-(pyrimidin-5-yl)ethyl group, a 1-(2-fluoropyrimidin-5-yl)ethyl group, a 1-(4-fluoropyrimidin-5-yl)ethyl group, a 1-(6-fluoropyrimidine-5-yl)ethyl group, a 1-(2-chloropyrimidin-5-yl)ethyl group, a 1-(4-chloropyrimidin-5-yl)ethyl group, a 1-(6-chloropyrimidin-5-yl)ethyl group, a 1-(2-bromopyrimidin-5-yl)ethyl group, a 1-(4-bromopyrimidin-5-yl)ethyl group, a 1-(6-bromopyrimidin-5-yl)ethyl group, a 1-(2-methylpyrimidin-5-yl)ethyl group, a 1-(4-methylpyrimidin-5-yl)ethyl group, a 1-(6-methylpyrimidin-5-yl)ethyl group, a 1-(2-trifluoromethylpyrimidin-5-yl)ethyl group, a 1-(4-trifluoromethylpyrimidin-5-yl)ethyl group, a 1-(6-trifluoromethylpyrimidin-5-yl)ethyl group, a 1-(2-methoxypyrimidin-5-yl)ethyl group, a 1-(4-methoxypyrimidin-5-yl)ethyl group, a 1-(6-methoxypyrimidin-5-yl)ethyl group,
a 1-(pyrazin-2-yl)ethyl group, a 1-(3-fluoropyrazin-2-yl)ethyl group, a 1-(5-fluoropyrazin-2-yl)ethyl group, a 1-(6-fluoropyrazine-2-yl)ethyl group, a 1-(3-chloropyrazin-2-yl)ethyl group, a 1-(5-chloropyrazin-2-yl)ethyl group, a 1-(6-chloropyrazin-2-yl)ethyl group, a 1-(3-bromopyrazin-2-yl)ethyl group, a 1-(5-bromopyrazin-2-yl)ethyl group, a 1-(6-bromopyrazin-2-yl)ethyl group, a 1-(3-methylpyrazin-2-yl)ethyl group, a 1-(5-methylpyrazin-2-yl)ethyl group, a 1-(6-methylpyrazin-2-yl)ethyl group, a 1-(3-trifluoromethylpyrazin-2-yl)ethyl group, a 1-(5-trifluoromethylpyrazin-2-yl)ethyl group, a 1-(6-trifluoromethylpyrazin-2-yl)ethyl group, a 1-(3-methoxypyrazin-2-yl)ethyl group, a 1-(5-methoxypyrazin-2-yl)ethyl group, a 1-(6-methoxypyrazin-2-yl)ethyl group,
a 3-methyl-1,2,4-oxadiazolyl group, a 5-fluoro-3-methyl-1,2,4-oxadiazolyl group, a 5-chloro-3-methyl-1,2,4-oxadiazolyl group, a 5-bromo-3-methyl-1,2,4-oxadiazolyl group, a 5-methyl-3-methyl-1,2,4-oxadiazolyl group, a 5-trifluoromethyl-3-methyl-1,2,4-oxadiazolyl group, a 5-methoxy-3-methyl-1,2,4-oxadiazolyl group, a thiazol-2-ylmethyl group, a thiazol-4-ylmethyl group, a thiazol-5-ylmethyl group,
a tetrahydrofuran-2-ylmethyl group, a tetrahydrofuran-3-ylmethyl group, a tetrahydro-2H-pyran-2-ylmethyl group, a tetrahydro-2H-pyran-4-ylmethyl group, a 1,3-dioxolan-2-ylmethyl group, or a 3-(dihydrofuran-2(3H)-one)methyl group,
more preferably a methyl group, an ethyl group, a propyl group, an isopropyl group, a butyl group, an isobutyl group, a sec-butyl group, a pentyl group, a cyanomethyl group, a 1-cyanoethyl group, a 2-cyanoethyl group, a cyclopropylmethyl group, a methoxymethyl group, an ethoxymethyl group, a methoxyethyl group, an ethoxyethyl group, an isopropyloxymethyl group, a benzyl group, a 1-phenethyl group, a 2-phenethyl group, a 4-cyanophenylmethyl group, a 2-fluorophenylmethyl group, a 3-fluorophenylmethyl group, a 4-fluorophenylmethyl group, a 2-chlorophenylmethyl group, a 3-chlorophenylmethyl group, a 3,5-dichlorophenylmethyl group, a 4-chlorophenylmethyl group, a 2-methylphenylmethyl group, a 3-methylphenylmethyl group, a 4-methylphenylmethyl group, a 2-trifluoromethylphenylmethyl group, a 3-trifluoromethylphenylmethyl group, a 4-trifluoromethylphenylmethyl group, a 3-cyclopropylpyridin-2-ylmethyl group, a 4-cyclopropylpyridin-2-ylmethyl group, a 5-cyclopropylpyridin-2-ylmethyl group, a 6-cyclopropylpyridin-2-ylmethyl group, a 2-methoxyphenylmethyl group, a 3-methoxyphenylmethyl group, a 4-methoxyphenylmethyl group, a 4-phenoxyphenylmethyl group, a 4-methanesulfonylphenylmethyl group, a 4-trifluoromethylsulfanylphenylmethyl group, a 4-trifluorosulfinylphenylmethyl group, a 4-trifluorosulfonylphenylmethyl group, a pyridin-2-ylmethyl group, a pyridin-2-ylmethyl-N-oxide group, a 3-chloropyridin-2-ylmethyl group, a 4-chloropyridin-2-ylmethyl group, a 5-chloropyridin-2-ylmethyl group, a 6-chloropyridin-2-ylmethyl group, a 3-bromopyridin-2-ylmethyl group, a 4-bromopyridin-2-ylmethyl group, a 5-bromopyridin-2-ylmethyl group, a 6-bromopyridin-2-ylmethyl group, a 3-methylpyridin-2-ylmethyl group, a 4-methylpyridin-2-ylmethyl group, a 5-methylpyridin-2-ylmethyl group, a 6-methylpyridin-2-ylmethyl group, a 3-trifluoromethylpyridin-2-ylmethyl group, a 4-trifluoromethylpyridin-2-ylmethyl group, a 5-trifluoromethylpyridine-2-ylmethyl group, a 6-trifluoromethylpyridin-2-ylmethyl group,
a 3-cyclopropylpyridin-2-ylmethyl group, a 4-cyclopropylpyridin-2-ylmethyl group, a 5-cyclopropylpyridin-2-ylmethyl group, a 6-cyclopropylpyridin-2-ylmethyl group, a 3-methoxypyridin-2-ylmethyl group, a 4-methoxypyridin-2-ylmethyl group, a 5-methoxypyridin-2-ylmethyl group, a 6-methoxypyridin-2-ylmethyl group, a pyridin-3-ylmethyl group, a 2-chloropyridin-3-ylmethyl group, a 4-chloropyridin-3-ylmethyl group, a 5-chloropyridin-3-ylmethyl group, a 6-chloropyridin-3-ylmethyl group, a 2-methylpyridin-3-ylmethyl group, a 4-methylpyridin-3-ylmethyl group, a 5-methylpyridin-3-ylmethyl group, a 6-methylpyridin-3-ylmethyl group, a 2-trifluoromethylpyridin-3-ylmethyl group, a 4-trifluoromethylpyridin-3-ylmethyl group, a 5-trifluoromethylpyridin-3-ylmethyl group, a 6-trifluoromethylpyridin-3-ylmethyl group, a pyridin-4-ylmethyl group, a 2-chloropyridin-4-ylmethyl group, a 3-chloropyridin-4-ylmethyl group, a 2-methylpyridin-4-ylmethyl group, a 3-methylpyridin-4-ylmethyl group, a 2-trifluoromethylpyridin-4-ylmethyl group, a 3-trifluoromethylpyridin-4-ylmethyl group, a pyridazin-3-ylmethyl group, a 4-chloropyridazin-3-ylmethyl group, a 5-chloropyridazin-3-ylmethyl group, a 6-chloropyridazin-3-ylmethyl group, a 4-methylpyridazin-3-ylmethyl group, a 5-methylpyridazin-3-ylmethyl group, a 6-methylpyridazin-3-ylmethyl group, a 4-trifluoromethylpyridazin-3-ylmethyl group, a 5-trifluoromethylpyridazin-3-ylmethyl group, a 6-trifluoromethylpyridazin-3-ylmethyl group, a pyridazin-4-ylmethyl group, a 3-chloropyridazin-4-ylmethyl group, a 5-chloropyridazin-4-ylmethyl group, a 6-chloropyridazin-4-ylmethyl group, a 3-methylpyridazin-4-ylmethyl group, a 5-methylpyridazin-4-ylmethyl group, a 6-methylpyridazin-4-ylmethyl group, a 3-trifluoromethylpyridazin-4-ylmethyl group, a 5-trifluoromethylpyridazin-4-ylmethyl group, a 6-trifluoromethylpyridazin-4-ylmethyl group, a 3-methoxypyridazin-4-ylmethyl group, a pyrimidin-2-ylmethyl group,
a 4-chloropyrimidin-2-ylmethyl group, a 5-chloropyrimidin-2-ylmethyl group, a 4-methylpyrimidin-2-ylmethyl group, a 5-methylpyrimidin-2-ylmethyl group, a 4-trifluoromethylpyrimidin-2-ylmethyl group, a 5-trifluoromethylpyrimidin-2-ylmethyl group, a pyrimidin-4-ylmethyl group, a 2-chloropyrimidin-4-ylmethyl group, a 5-chloropyrimidin-4-ylmethyl group, a 6-chloropyrimidin-4-ylmethyl group, a 2-methylpyrimidin-4-ylmethyl group, a 5-methylpyrimidin-4-ylmethyl group, a 6-methylpyrimidin-4-ylmethyl group, a 2-trifluoromethylpyrimidin-4-ylmethyl group, a 5-trifluoromethylpyrimidin-4-ylmethyl group, a 6-trifluoromethylpyrimidin-4-ylmethyl group, a pyrimidin-5-ylmethyl group, a 2-chloropyrimidin-5-ylmethyl group, a 4-chloropyrimidin-5-ylmethyl group, a 6-chloropyrimidin-5-ylmethyl group, a 2-methylpyrimidin-5-ylmethyl group, a 4-methylpyrimidin-5-ylmethyl group, a 6-methylpyrimidin-5-ylmethyl group, a 2-trifluoromethylpyrimidin-5-ylmethyl group, a 4-trifluoromethylpyrimidin-5-ylmethyl group, a 6-trifluoromethylpyrimidin-5-ylmethyl group, a pyrazin-2-ylmethyl group, a 3-chloropyrazin-2-ylmethyl group, a 5-chloropyrazin-2-ylmethyl group, a 6-chloropyrazin-2-ylmethyl group, a 3-methylpyrazin-2-ylmethyl group, a 5-methylpyrazin-2-ylmethyl group, a 6-methylpyrazin-2-ylmethyl group, a 3-trifluoromethylpyrazin-2-ylmethyl group, a 5-trifluoromethylpyrazin-2-ylmethyl group, a 6-trifluoromethylpyrazin-2-ylmethyl group, a 1-(pyridin-2-yl)ethyl group, a 1-(3-chloropyridin-2-yl)ethyl group, a 1-(-chloropyridin-2-yl)ethyl group, a 1-(5-chloropyridin-2-yl)ethyl group, a 1-(6-chloropyridin-2-yl)ethyl group, a 1-(3-methylpyridin-2-yl)ethyl group, a 1-(6-methylpyridin-2-yl)ethyl group, a 1-(5-methylpyridin-2-yl)ethyl group, a 1-(6-methylpyridin-2-yl)ethyl group, a 1-(3-trifluoromethylpyridin-2-yl)ethyl group, a trifluoromethylpyridin-2-yl)ethyl group, a 1-(5-trifluoromethylpyridin-2-yl)ethyl group, a 1-(6-trifluoromethylpyridin-2-yl)ethyl group,
a 1-(pyridin-3-yl)ethyl group, a 1-(2-chloropyridin-3-yl)ethyl group, a 1-(4-chloropyridin-3-yl)ethyl group, a 1-(5-chloropyridin-3-yl)ethyl group, a 1-(6-chloropyridin-3-yl)ethyl group, a 1-(2-methylpyridin-3-yl)ethyl group, a 1-(4-methylpyridin-3-yl)ethyl group, a 1-(5-methylpyridin-3-yl)ethyl group, a 1-(6-methylpyridin-3-yl)ethyl group, a 1-(2-trifluoromethylpyridin-3-yl)ethyl group, a 1-(4-trifluoromethylpyridin-3-yl)ethyl group, a 1-(5-trifluoromethylpyridin-3-yl)ethyl group, a 1-(6-trifluoromethylpyridin-3-yl)ethyl group, a 1-(pyridin-4-yl)ethyl group, a 1-(2-chloropyridin-4-yl)ethyl group, a 1-(3-chloropyridin-4-yl)ethyl group, a 1-(2-methylpyridin-4-yl)ethyl group, a 1-(3-methylpyridin-4-yl)ethyl group, a 1-(2-trifluoromethylpyridin-4-yl)ethyl group, a 1-(3-trifluoromethylpyridin-4-yl)ethyl group, a 1-(pyridazin-3-yl)ethyl group, a 1-(4-chloropyridazin-3-yl)methyl group, a 1-(5-chloropyridazine-3-yl)ethyl group, a 1-(6-chloropyridazin-3-yl)ethyl group, a 1-(4-methylpyridazin-3-yl)ethyl group, a 1-(5-methylpyridazin-3-yl)ethyl group, a 1-(6-methylpyridazin-3-yl)ethyl group, a 1-(4-trifluoromethylpyridazin-3-yl)ethyl group, a 1-(5-trifluoromethylpyridazin-3-yl)ethyl group, a 1-(6-trifluoromethylpyridazin-3-yl)ethyl group, a 1-(pyridazin-4-yl)ethyl group, a 1-(3-chloropyridazin-4-yl)ethyl group, a 1-(5-chloropyridazin-4-yl) ethyl group, a 1-(6-chloropyridazin-4-yl)ethyl group, a 1-(3-methylpyridazin-4-yl)ethyl group, a 1-(5-methylpyridazin-4-yl)ethyl group, a 1-(6-methylpyridazin-4-yl)ethyl group, a 1-(3-trifluoromethylpyridazin-4-yl)ethyl group, a 1-(5-trifluoromethylpyridazin-4-yl)ethyl group, a 1-(6-trifluoromethylpyridazin-4-yl)ethyl group, a 1-(pyrimidin-2-yl)ethyl group, a 1-(4-chloropyrimidin-2-yl)ethyl group, a 1-(5-chloropyrimidin-2-yl)ethyl group, a 1-(4-methylpyrimidin-2-yl)ethyl group, a 1-(5-methylpyrimidin-2-yl)ethyl group, a 1-(4-trifluoromethylpyrimidin-2-yl)ethyl group, a 1-(5-trifluoromethylpyrimidin-2-yl)ethyl group, a 1-(pyrimidin-4-yl)ethyl group, a 1-(2-chloropyrimidin-4-yl)ethyl group, a 1-(5-chloropyrimidin-4)-yl)ethyl group, a 1-(6-chloropyrimidin-4-yl)ethyl group, a 1-(2-methylpyrimidin-4-yl)ethyl group, a 1-(5-methylpyrimidin-4-yl)ethyl group, a 1-(6-methylpyrimidin-4-yl)ethyl group, a 1-(2-trifluoromethylpyrimidin-4-yl)ethyl group, a 1-(5-trifluoromethylpyrimidin-4-yl)ethyl group, a 1-(6-trifluoromethylpyrimidin-4-yl)ethyl group, a 1-(pyrimidin-5-yl)ethyl group, a 1-(2-chloropyrimidin-5-yl)ethyl group, a 1-(4-chloropyrimidin-5-yl)ethyl group, a 1-(6-chloropyrimidin-5-yl)ethyl group, a 1-(2-methylpyrimidin-5-yl)ethyl group, a 1-(4-methylpyrimidin-5-yl)ethyl group, a 1-(6-methylpyrimidin-5-yl)ethyl group, a 1-(2-trifluoromethylpyrimidin-5-yl)ethyl group, a 1-(4-trifluoromethylpyrimidin-5-yl)ethyl group, a 1-(6-trifluoromethylpyrimidin-5-yl)ethyl group, a 1-(pyrazin-2-yl)ethyl group, a 1-(3-chloropyrazin-2-yl)ethyl group, a 1-(5-chloropyrazin-2-yl)ethyl group, a 1-(6-chloropyrazin-2-yl)ethyl group, a 1-(3-methylpyrazin-2-yl)ethyl group, a 1-(5-methylpyrazin-2-yl)ethyl group, a 1-(6-methylpyrazin-2-yl)ethyl group, a 1-(3-trifluoromethylpyrazin-2-yl)ethyl group, a 1-(5-trifluoromethylpyrazin-2-yl)ethyl group, a 1-(6-trifluoromethylpyrazin-2-yl)ethyl group, a 3-methyl-1,2,4-oxadiazolyl group, a 5-trifluoromethyl-3-methyl-1,2,4-oxadiazolyl group, a thiazol-2-ylmethyl group, a thiazol-4-ylmethyl group, a thiazol-5-ylmethyl group, a propan-2-one group, a methoxycarbonylmethyl group, a methoxycarbonylethyl group, an ethoxycarbonylmethyl group, an ethoxycarbonylethyl group, a dimethylaminocarbonylmethyl group, an ethylmethylaminocarbonylmethyl group, a methylsulfanylmethyl group, a methylsulfinylmethyl group, a methylsulfonylmethyl group, an ethylsulfanylmethyl group, an ethylsulfinylmethyl group, an ethylsulfonylmethyl group, a trifluoromethylsulfanylmethyl group, a trifluoromethylsulfinylmethyl group, a trifluorosulfonylmethyl group, a tetrahydrofuran-2-ylmethyl group, a tetrahydrofuran-3-ylmethyl group, a tetrahydro-2H-pyran-2-ylmethyl group, a tetrahydro-2H-pyran-4-ylmethyl group, a 1,3-dioxolan-2-ylmethyl group, or a 3-(dihydrofuran-2(3H)-one)methyl group, and still more preferably a methyl group, an ethyl group, a propyl group, an isopropyl group, a butyl group, an isobutyl group, a sec-butyl group, a cyanomethyl group, a 2-cyanoethyl group, a cyclopropylmethyl group, a methoxymethyl group, an ethoxymethyl group, a methoxyethyl group, an ethoxyethyl group, or a pyrimidin-5-ylmethyl group.

A methyl group, an ethyl group, a propyl group, or a pyrimidin-5-ylmethyl group is particularly preferable.

The “C1 to C6 haloalkyl group” in R2 in Formula (1-2) has the same meaning as above, and is preferably a difluoromethyl group, a trifluoromethyl group, a 2,2-difluoroethyl group, a 2,2,2-trifluoroethyl group, a 1,2,2,2-tetrafluoroethyl group, a 1,1,2,2,2-pentafluoroethyl group, a 2,2,3,3,3-pentafluoropropyl group, a 1,1,2,2,3,3,3-heptafluoropropyl group, a 2,2,3,3,4,4,4-heptafluorobutyl group, or a 2,2,3,3,4,4,5,5,5-nonafluoropentyl group, more preferably a difluoromethyl group, a 2,2,2-trifluoroethyl group, a 1,1,2,2,2-pentafluoroethyl group, a 1,1,2,2,3,3,3-heptafluoropropyl group, a 2,2,3,3,4,4,4-heptafluorobutyl group, or a 2,2,3,3,4,4,5,5,5-nonafluoropentyl group, still more preferably a difluoromethyl group, a 2,2,2-trifluoroethyl group, a 2,2,3,3,3-pentafluoropropyl group, or a 2,2,3,3,4,4,4-heptafluorobutyl group, and particularly preferably a 2,2,2-trifluoroethyl group.

The C3 to C8 cycloalkyl group of the “C3 to C8 cycloalkyl group optionally substituted with a substituent B” in R2 in Formula (1-2) has the same meaning as above, and is preferably a cyclopropyl group, a cyclobutyl group, a cyclopentyl group, or a cyclohexyl group, more preferably a cyclopropyl group or a cyclobutyl group, and particularly preferably a cyclopropyl group. When having a substituent B, one hydrogen atom in the C3 to C8 cycloalkyl group is substituted with the substituent B. The “C3 to C8 cycloalkyl group optionally substituted with a substituent B” is preferably a cyclopropyl group, a cyclobutyl group, a cyclopentyl group, a cyclohexyl group, a 1-(cyclopropane-1-carbonitrile) group, a 2-(cyclopropane-1-carbonitrile) group, a 1-(1-nitrocyclopropyl) group, a 1-(2-nitrocyclopropyl) group, a 1-(1-cyclopropylcyclopropyl) group, a 1-(2-cyclopropylcyclopropyl) group, a 1-(2-methoxycyclopropyl) group, a 1-(2-ethoxycyclopropyl) group, a 1-(1-phenylcyclopropyl) group, a 1-(2-phenylcyclopropyl) group, a 1-(cyclopropylethane-1-one) group, a 2-(cyclopropylethan-1-one) group, a 1-(1-methoxycarbonylcyclopropyl) group, a 1-(2-methoxycarbonylcyclopropyl) group, a 1-(1-ethoxycarbonylcyclopropyl) group, or a 1-(2-ethoxycarbonylcyclopropyl) group,
more preferably a cyclopropyl group, a cyclobutyl group, a 1-(cyclopropane-1-carbonitrile) group, a 2-(cyclopropane-1-carbonitrile) group, a 1-(1-nitrocyclopropyl) group, a 1-(2-nitrocyclopropyl) group, a 1-(1-methoxycarbonylcyclopropyl) group, a 1-(2-methoxycarbonylcyclopropyl) group, a 1-(1-ethoxycarbonylcyclopropyl) group, or a 1-(2-ethoxycarbonylcyclopropyl) group,
still more preferably a cyclopropyl group, a 1-(cyclopropane-1-carbonitrile) group, or a 1-(1-methoxycarbonylcyclopropyl) group, and
particularly preferably a cyclopropyl group.

The C2 to C6 alkenyl group of the “C2 to C6 alkenyl group optionally substituted with a substituent A” in R2 in Formula (1-2) has the same meaning as above, and is preferably a vinyl group, a 1-propenyl group, an allyl group, a 2-methylallyl group, a 1-butenyl group, a 2-butenyl group, or a 3-butenyl group, more preferably a vinyl group, a 1-propenyl group, an allyl group, a 2-methylallyl group, or a 3-butenyl group, and particularly preferably an allyl group, a 2-methylallyl group, or a 3-butenyl group. When having the substituent described in the substituent A, a hydrogen atom in the C2 to C6 alkenyl group is optionally substituted with the substituent described in the substituent A.

The “C2 to C6 haloalkenyl group” in R2 in Formula (1-2) has the same meaning as above, is preferably a 2-fluorovinyl group, a 2,2-difluorovinyl group, a 3-fluoroallyl group, or a 3,3-difluoroallyl group, more preferably a 3-fluoroallyl group or a 3,3-difluoroallyl group, and particularly preferably a 3,3-difluoroallyl group.

The C2 to C6 alkynyl group of the “C2 to C6 alkynyl group optionally substituted with a substituent A” in R2 in Formula (1-2) has the same meaning as above, and is preferably an ethynyl group, a 1-propynyl group, a propargyl group, a 1-butynyl group, a 2-butynyl group, or a 3-butynyl group, more preferably an ethynyl group, a 1-propynyl group, a propargyl group, or a 2-butynyl group, and particularly preferably a propargyl group or a 2-butynyl group. When having a substituent A, one hydrogen atom in the C2 to C6 alkynyl group is substituted with the substituent A.

The “C2 to C6 haloalkynyl group” in R2 in Formula (1-2) has the same meaning as above, and is preferably a 3,3-difluoro-1-propynyl group, a 3,3,3-trifluoro-1-propynyl group, a 4,4-difluoro-1-butynyl group, a 4,4-difluoro-2-butynyl group, a 4,4,4-trifluoro-1-butynyl group, or a 4,4,4-trifluoro-2-butynyl group, more preferably a 3,3-difluoro-1-propynyl group, a 3,3,3-trifluoro-1-propynyl group, or a 4,4,4-trifluoro-1-butynyl group, and particularly preferably a 3,3-difluoro-1-propynyl group or a 3,3,3-trifluoro-1-propynyl group.

In the “phenyl group optionally substituted with 0 to 5 substituents B” in R2 in Formula (1-2), hydrogen atoms in the phenyl group are optionally substituted with 1 to 5 independent substituents B. The “phenyl group optionally substituted with 0 to 5 substituents B” is preferably a phenyl group, a cyanophenyl group, a fluorophenyl group, a chlorophenyl group, a tolyl group, a methoxyphenyl group, a phenoxyphenyl group, a methylsulfanylphenyl group, a methylsulfinylphenyl group, a methylsulfonylphenyl group, a trifluoromethylsulfanylphenyl group, a trifluoromethylsulfinylphenyl group, or a trifluoromethylsulfonylphenyl group, more preferably a phenyl group, a cyanophenyl group, a fluorophenyl group, a chlorophenyl group, a tolyl group, a methoxyphenyl group, a methylsulfonylphenyl group, a trifluoromethylsulfanylphenyl group, a trifluoromethylsulfinylphenyl group, or a trifluoromethylsulfonylphenyl group, and particularly preferably a phenyl group.

The 5- or 6-membered aromatic heterocyclic group of the “5- or 6-membered aromatic heterocyclic group optionally substituted with 0 to 4 substituents B” in R2 in Formula (1-2) has the same meaning as above, and is preferably a pyridyl group, a pyridazinyl group, a pyrimidinyl group, a pyrazinyl group, a thienyl group, a thiazolyl group, an isothiazolyl group, an imidazolyl group, a pyrazolyl group, an oxazolyl group, an isoxazolyl group, a triazolyl group, an oxadiazolyl group, a thiadiazolyl group, or a tetrazolyl group, more preferably a pyridyl group, a pyridazinyl group, a pyrimidinyl group, a pyrazinyl group, a thienyl group, a thiazolyl group, an imidazolyl group, a pyrazolyl group, an oxazolyl group, an isoxazolyl group, a triazolyl group, or an oxadiazolyl group, and particularly preferably a pyridyl group or a pyrimidinyl group.

The “5- or 6-membered aromatic heterocyclic group optionally substituted with 0 to 4 substituents B” is preferably a pyridyl group, a 2-cyanopyridyl group, a 3-cyanopyridyl group, a 4-cyanopyridyl group, a 2-fluoropyridyl group, a 3-fluoropyridyl group, a 4-fluoropyridyl group, a 2-chloropyridyl group, a 3-chloropyridyl group, a 4-chloropyridyl group, a 2-bromopyridyl group, a 3-bromopyridyl group, a 4-bromopyridyl group, a 2-iodopyridyl group, a 3-iodopyridyl group, a 4-iodopyridyl group, a 2-methylpyridyl group, a 3-methylpyridyl group, a 4-methylpyridyl group, a 2-trifluoromethylpyridyl group, a 3-trifluoromethylpyridyl group, a 4-trifluoromethylpyridyl group, a 2-methoxypyridyl group, a 3-methoxypyridyl group, a 4-methoxypyridyl group, a pyridazinyl group, a 3-cyanopyridazinyl group, a 4-cyanopyridazinyl group, a 3-fluoropyridazinyl group, a 4-fluoropyridazinyl group, a 3-chloropyridazinyl group, a 4-chloropyridazinyl group, a 3-bromopyridazinyl group, a 4-bromopyridazinyl group, a 3-iodopyridazinyl group, a 4-iodopyridazinyl group, a 3-methylpyridazinyl group, a 4-methylpyridazinyl group, a 3-trifluoromethylpyridazinyl group, a 4-trifluoromethylpyridazinyl group, a 3-methoxypyridazinyl group, a 4-methoxypyridazinyl group, a pyrimidinyl group, a 2-cyanopyrimidinyl group, a 4-cyanopyrimidinyl group, a 5-cyanopyrimidinyl group, a 2-fluoropyrimidinyl group, a 4-fluoropyrimidinyl group, a 5-fluoropyrimidinyl group, a 2-chloropyrimidinyl group, a 4-chloropyrimidinyl group, a 5-chloropyrimidinyl group, a 2-bromopyrimidinyl group, a 4-bromopyrimidinyl group, a 5-bromopyrimidinyl group, a 2-iodopyrimidinyl group, a 4-iodopyrimidinyl group, a 5-iodopyrimidinyl group, a 2-methylpyrimidinyl group, a 4-methylpyrimidinyl group, a 5-methylpyrimidinyl group, a 2-trifluoromethylpyrimidinyl group, a 4-trifluoromethylpyrimidinyl group, a 5-trifluoromethylpyrimidinyl group, a 2-methoxypyrimidinyl group, a 4-methoxypyrimidinyl group, a 5-methoxypyrimidinyl group, a pyrazinyl group, a 2-cyanopyrazinyl group, a 2-fluoropyrazinyl group, a 2-chloropyrazinyl group, a 2-bromopyrazinyl group, a 2-iodopyrazinyl group, a 2-methylpyrazinyl group, a 2-trifluoromethylpyrazinyl group, or a 2-methoxypyrazinyl group, more preferably a pyridyl group, a 2-cyanopyridyl group, a 3-cyanopyridyl group, a 4-cyanopyridyl group, a 2-fluoropyridyl group, a 3-fluoropyridyl group, a 4-fluoropyridyl group, a 2-chloropyridyl group, a 3-chloropyridyl group, a 4-chloropyridyl group, a 2-bromopyridyl group, a 3-bromopyridyl group, a 4-bromopyridyl group, a 2-iodopyridyl group, a 3-iodopyridyl group, a 4-iodopyridyl group, a 2-methylpyridyl group, a 3-methylpyridyl group, a 4-methylpyridyl group, a 2-trifluoromethylpyridyl group, a 3-trifluoromethylpyridyl group, a 4-trifluoromethylpyridyl group, a 2-methoxypyridyl group, a 3-methoxypyridyl group, a 4-methoxypyridyl group, a pyrimidyl group, a 2-cyanopyrimidinyl group, a 4-cyanopyrimidinyl group, a 5-cyanopyrimidinyl group, a 2-fluoropyrimidinyl group, a 4-fluoropyrimidinyl group, a 5-fluoropyrimidinyl group, a 2-chloropyrimidinyl group, a 4-chloropyrimidinyl group, a 5-chloropyrimidinyl group, a 2-bromopyrimidinyl group, a 4-bromopyrimidinyl group, a 5-bromopyrimidinyl group, a 2-iodopyrimidinyl group, a 4-iodopyrimidinyl group, a 5-iodopyrimidinyl group, a 2-methylpyrimidinyl group, a 4-methylpyrimidinyl group, a 5-methylpyrimidinyl group, a 2-trifluoromethylpyrimidinyl group, a 4-trifluoromethylpyrimidinyl group, a 5-trifluoromethylpyrimidinyl group, a 2-methoxypyrimidinyl group, a 4-methoxypyrimidinyl group, or a 5-methoxypyrimidinyl group, and
particularly preferably a pyridyl group, a 3-trifluoromethylpyridyl group, or a pyrimidyl group.

Each term of Rx1Rx2NC(═O)— (where Rx1 and Rx2 each independently represent a hydrogen atom, a hydroxyl group, a cyano group, a C1 to C6 alkyl group optionally substituted with a substituent A, a C1 to C6 haloalkyl group, a C3 to C8 cycloalkyl group optionally substituted with a substituent B, a C2 to C6 alkenyl group optionally substituted with a substituent A, a C2 to C6 haloalkenyl group, a C2 to C6 alkynyl group optionally substituted with a substituent A, a C2 to C6 haloalkynyl group, a C1 to C6 alkoxy group, a C1 to C6 haloalkoxy group, a C3 to C8 cycloalkoxy group, a phenyl group optionally substituted with 0 to 5 substituents B, or a 5- or 6-membered aromatic heterocyclic group optionally substituted with 0 to 4 substituents B, or Rx1 and Rx2 together with a nitrogen atom to which they are bonded form an aziridinyl group, an azetidinyl group, a pyrrolidinyl group, a piperidinyl group, a piperazinyl group, a morpholinyl group, a thiomorpholinyl group, a dioxothiomorpholinyl group, a thiazolidinyl group, an azepanyl group, or an azocanyl group) in R2 in Formula (1-2) has the same meaning as above.

The “Rx1Rx2NC(═O)—” is preferably a carbamoyl group, a methylcarbonyl group, an ethylcarbamoyl group, a propylcarbamoyl group, an isopropylcarbamoyl group, a butylcarbamoyl group, an isobutylcarbamoyl group, a sec-butylcarbamoyl group, a t-butylcarbamoyl group, a pentylcarbamoyl group, an isopentylcarbamoyl group, a 2-methylbutylcarbamoyl group, a neopentylcarbamoyl group, a 1-ethylpropylcarbamoyl group, a hexylcarbamoyl group, a methoxymethylcarbamoyl group, an ethoxymethylcarbamoyl group, a propyloxymethylcarbamoyl group, an isopropyloxymethylcarbamoyl group, a 2-methoxyethylcarbamoyl group, a 2-ethoxyethylcarbamoyl group, a cyanomethylcarbamoyl group, a 1-cyanoethylcarbamoyl group, a 2-cyanoethylcarbamoyl group, a cyclopropylmethylcarbamoyl group, a cyclobutylmethylcarbamoyl group,
a benzylcarbamoyl group, a 1-phenethylcarbamoyl group, a 1,1-difluoroethylcarbamoyl group, a 2,2-difluoroethylcarbamoyl group, a 2,2,2-trifluoroethylcarbamoyl group, a 2,2,3,3,3-pentafluoropropylcarbamoyl group, a 2,2,3,3,4,4,4-heptafluorobutylcarbamoyl group, a 3,3,4,4,5,5,5-heptafluoropentylcarbamoyl group, a cyclopropylcarbamoyl group, a 1-cyanocyclopropylcarbamoyl group, an allylcarbamoyl group, a 3,3-difluoroallylcarbamoyl group, a propargylcarbamoyl group, a methoxycarbamoyl group, a trifluoroethoxycarbamoyl group, a phenylcarbamoyl group, a 4-cyanophenylcarbamoyl group, a 2-fluorophenylcarbamoyl group, a 3-fluorophenylcarbamoyl group, a 4-fluorophenylcarbamoyl group, a 2-chlorophenylcarbamoyl group, a 3-chlorophenylcarbamoyl group, a 4-chlorophenylcarbamoyl group, a 2-trifluoromethylphenylcarbamoyl group, a 3-trifluoromethylphenylcarbamoyl group, a 4-trifluoromethylphenylcarbamoyl group, a 2-methoxyphenylcarbamoyl group, a 3-methoxyphenylcarbamoyl group, a 4-methoxyphenylcarbamoyl group, a pyrrolidinecarbonyl group, a piperidinecarbonyl group, a piperazinecarbonyl group, a morpholinecarbonyl group, a thiomorpholinecarbonyl group, a dioxothiomorpholinecarbonyl group, a thiazolidinecarbonyl group, an azepanecarbonyl group, an azocancarbonyl group, a dimethylcarbamoyl group, an ethylmethylcarbamoyl group, a diethylcarbamoyl group, a methylpropylcarbamoyl group, an ethylpropylcarbamoyl group, an isopropylmethylcarbamoyl group, an isopropylethylcarbamoyl group, a diisopropylcarbamoyl group, a butylmethylcarbamoyl group, a butylethylcarbamoyl group, a sec-butylmethylcarbamoyl group, a sec-butylethylcarbamoyl group, an isobutylmethylcarbamoyl group, an isobutylethyl carbamoyl group,
a t-butylmethylcarbamoyl group, a t-butylethylcarbamoyl group, a methylpentylcarbamoyl group, an ethylpentylate carbamoyl group, an isopentylmethylcarbamoyl group, an isopentylethylcarbamoyl group, a methyl(2-methylbutyl)carbamoyl group, an ethyl(2-methylbutyl)carbamoyl group, a hexylmethylcarbamoyl group, a hexylethylcarbamoyl group, a (methoxymethyl)methylcarbamoyl group, an (ethoxymethyl)methylcarbamoyl group, a (methoxyethyl)methylcarbamoyl group, an (ethoxyethyl)methylcarbamoyl group, a (propyloxymethyl)methylcarbamoyl group, an (isopropyloxymethyl)methylcarbamoyl group, a (methoxymethyl)ethylcarbamoyl group, an (ethoxymethyl)ethylcarbamoyl group, a (cyanomethyl)methylcarbamoyl group, a (cyanomethyl)ethylcarbamoyl group, a (cyclopropylmethyl)methylcarbamoyl group, a (cyclopropylmethyl)ethylcarbamoyl group, a (benzyl)methylcarbamoyl group, a methyl(pyridin-3-ylmethyl)carbamoyl group, a methyl(2,2,2-trifluoroethyl)carbamoyl group, a methyl(2,2,3,3,3-pentafluoropropyl)carbamoyl group, a methyl(2,2,3,3,4,4,4-heptafluorobutyl)carbamoyl group, a methyl(3,3,4,4,5,5,5-heptafluoropentyl)carbamoyl group, a (cyclopropyl)methylcarbamoyl group, a (cyclopropyl)ethylcarbamoyl group, a (1-cyanocyclopropyl)methylcarbamoyl group, a (1-cyanocyclopropyl)ethylcarbamoyl group, a (cyclobutyl)methylcarbamoyl group, a (cyclopentyl)methylcarbamoyl group, a (cyclohexyl)methylcarbamoyl group, an (allyl)methylcarbamoyl group, a methyl(2-propyn-1-yl)carbamoyl group, an ethyl(2-propyn-1-yl)carbamoyl group, a (methoxy)methylcarbamoyl group, an (ethoxy)methylcarbamoyl group, a (trifluoroethoxy)methylcarbamoyl group, a methylphenylcarbamoyl group, a diphenylcarbamoyl group, a methyl(pyridin-2-yl)carbamoyl group,
a methyl(6-methyl-pyridin-2-yl)carbamoyl group, a methyl(pyridin-3-yl)carbamoyl group, or a methyl(pyridin-4-yl)carbamoyl group,
more preferably a methylcarbonyl group, an ethylcarbamoyl group, a pyrrolidinecarbonyl group, a piperidinecarbonyl group, a piperazinecarbonyl group, a morpholinecarbonyl group, a thiomorpholinecarbonyl group, a dioxothiomorpholinecarbonyl group, a thiazolidinecarbonyl group, an azepanecarbonyl group, an azocancarbonyl group, a dimethylcarbamoyl group, an ethylmethylcarbamoyl group, a diethylcarbamoyl group, a methylpropylcarbamoyl group, an ethylpropylcarbamoyl group, an isopropylmethylcarbamoyl group, an isopropylethylcarbamoyl group, a diisopropylcarbamoyl group, a butylmethylcarbamoyl group, a butylethylcarbamoyl group, a sec-butylmethylcarbamoyl group, a sec-butylethylcarbamoyl group, an isobutylmethylcarbamoyl group, an isobutylethylcarbamoyl group, a t-butylmethylcarbamoyl group, a t-butylethylcarbamoyl group, a methylpentylcarbamoyl group, an ethylpentylate carbamoyl group, an isopentylmethylcarbamoyl group, an isopentylethylcarbamoyl group, a methyl(2-methylbutyl)carbamoyl group, an ethyl(2-methylbutyl)carbamoyl group, a hexylmethylcarbamoyl group, a hexylethylcarbamoyl group, a (methoxymethyl)methylcarbamoyl group, an (ethoxymethyl)methylcarbamoyl group, a (methoxyethyl)methylcarbamoyl group, an (ethoxyethyl)methylcarbamoyl group, a (propyloxymethyl)methylcarbamoyl group, an (isopropyloxymethyl)methylcarbamoyl group, a (methoxymethyl)ethylcarbamoyl group, an (ethoxymethyl)ethylcarbamoyl group, a (cyanomethyl)methylcarbamoyl group, a (cyanomethyl)ethylcarbamoyl group, a (cyclopropylmethyl)methylcarbamoyl group, a (cyclopropylmethyl)ethylcarbamoyl group, a (benzyl)methylcarbamoyl group, a methyl(pyridin-3-ylmethyl)carbamoyl group, a methyl(2,2,2-trifluoroethyl)carbamoyl group, a methyl(2,2,3,3,3-pentafluoropropyl)carbamoyl group, a methyl(2,2,3,3,4,4,4-heptafluorobutyl)carbamoyl group, a methyl(3,3,4,4,5,5,5-heptafluoropentyl)carbamoyl group, a (cyclopropyl)methylcarbamoyl group, a (cyclopropyl)ethylcarbamoyl group, a (1-cyanocyclopropyl)methylcarbamoyl group, a (1-cyanocyclopropyl)ethylcarbamoyl group, a (cyclobutyl)methylcarbamoyl group, a (cyclopentyl)methylcarbamoyl group, a (cyclohexyl)methylcarbamoyl group, an (allyl)methylcarbamoyl group, a methyl(2-propyn-1-yl)carbamoyl group, an ethyl(2-propyn-1-yl)carbamoyl group, a (methoxy)methylcarbamoyl group, an (ethoxy)methylcarbamoyl group, a (trifluoroethoxy)methylcarbamoyl group, a methylphenylcarbamoyl group, a diphenylcarbamoyl group, a methyl(pyridin-2-yl)carbamoyl group, a methyl(6-methyl-pyridin-2-yl)carbamoyl group, a methyl(pyridin-3-yl)carbamoyl group, or a methyl(pyridin-4-yl)carbamoyl group,
still more preferably an ethylcarbamoyl group, a pyrrolidinecarbonyl group, a piperidinecarbonyl group, a morpholinecarbonyl group, a thiomorpholinecarbonyl group, a dioxothiomorpholinecarbonyl group, a thiazolidinecarbonyl group, an azepanecarbonyl group, an azocancarbonyl group, a dimethylcarbamoyl group, an ethylmethylcarbamoyl group, a diethylcarbamoyl group, a methylpropylcarbamoyl group, an isopropylmethylcarbamoyl group, a diisopropylcarbamoyl group, a butylmethylcarbamoyl group, a butylethylcarbamoyl group, a sec-butylmethylcarbamoyl group, a sec-butylethylcarbamoyl group, an isobutylmethylcarbamoyl group, an isobutylethylcarbamoyl group, a t-butylmethylcarbamoyl group, a (methoxyethyl)methylcarbamoyl group, a (cyanomethyl)methylcarbamoyl group, a (cyanomethyl)ethylcarbamoyl group, a (cyclopropylmethyl)methylcarbamoyl group, a (cyclo propylmethyl)ethylcarbamoyl group, a (benzyl)methylcarbamoyl group, a methyl(pyridin-3-ylmethyl)carbamoyl group, a methyl(2,2,2-trifluoroethyl)carbamoyl group, a (cyclopropyl)methylcarbamoyl group, a (cyclopentyl)methylcarbamoyl group, a (cyclohexyl)methylcarbamoyl group, an (allyl)methylcarbamoyl group, a methyl(2-propyn-1-yl)carbamoyl group, an ethyl(2-propyn-1-yl)carbamoyl group, a (methoxy)methylcarbamoyl group, an (ethoxy)methylcarbamoyl group, a (trifluoroethoxy)methylcarbamoyl group, a methyl phenylcarbamoyl group, a diphenylcarbamoyl group, a methyl(pyridin-2-yl)carbamoyl group, a methyl(6-methyl-pyridin-2-yl)carbamoyl group, a methyl(pyridin-3-yl)carbamoyl group, or a methyl(pyridin-4-yl)carbamoyl group, and
particularly preferably an ethylcarbamoyl group, a pyrrolidinecarbonyl group, a piperidinecarbonyl group, a morpholinecarbonyl group, a thiomorpholinecarbonyl group, a thiazolidinecarbonyl group, an azepanecarbonyl group, an azocancarbonyl group, a dimethylearbamoyl group, an ethylmethylcarbamoyl group, a diethylcarbamoyl group, a methylpropylcarbamoyl group, an isopropylmethylcarbamoyl group, a diisopropylcarbamoyl group, a butylmethylcarbamoyl group, an isobutylmethylcarbamoyl group, a t-butylmethylcarbamoyl group, a (methoxyethyl)methylcarbamoyl group, a (cyanomethyl)methylcarbamoyl group, a (cyclopropylmethyl)methylcarbamoyl group, a methyl(pyridin-3-ylmethyl)carbamoyl group, a methyl(2,2,2-trifluoroethyl)carbamoyl group, a (cyclopropyl)methylcarbamoyl group, a methyl(2-propyn-1-yl)carbamoyl group, a (methoxy)methylcarbamoyl group, a methylphenylcarbamoyl group, a diphenylcarbamoyl group, a methyl(pyridin-2-yl)carbamoyl group, a methyl(6-methyl-pyridin-2-yl)carbamoyl group, a methyl(pyridin-3-yl)carbamoyl group, or a methyl(pyridin-4-yl)carbamoyl group.

Each term of “Rx3C(═O)—” (where Rx3 represents a hydrogen atom, a hydroxyl group, a halogen atom, a C1 to C6 alkyl group optionally substituted with a substituent A, a C1 to C6 haloalkyl group, a C3 to C8 cycloalkyl group optionally substituted with a substituent B, a C2 to C6 alkenyl group optionally substituted with a substituent A, a C2 to C6 haloalkenyl group, a C2 to C6 alkynyl group optionally substituted with a substituent A, a C2 to C6 haloalkynyl group, a phenyl group optionally substituted with 0 to 5 substituents B, or a 5- or 6-membered aromatic heterocyclic group optionally substituted with 0 to 4 substituents B) in R2 in Formula (1-2) has the same meaning as above.

The “Rx3C(═O)—” is preferably an acetyl group, a 1-cyanoacetyl group, a 1-methoxyacetyl group, a propionyl group, an isobutyryl group, a difluoroacetyl group, a trifluoroacetyl group, a benzoyl group, a 4-fluorobenzoyl group, or a 4-trifluorobenzoyl group, more preferably an acetyl group, a propionyl group, a difluoroacetyl group, a trifluoroacetyl group, or a benzoyl group, and particularly preferably an acetyl group, a trifluoroacetyl group, or a benzoyl group.

Each term of “Rx3OC(═O)—” (where Rx3 represents a hydrogen atom, a hydroxyl group, a halogen atom, a C1 to C6 alkyl group optionally substituted with a substituent A, a C1 to C6 haloalkyl group, a C3 to C8 cycloalkyl group optionally substituted with a substituent B, a C2 to C6 alkenyl group optionally substituted with a substituent A, a C2 to C6 haloalkenyl group, a C2 to C6 alkynyl group optionally substituted with a substituent A, a C2 to C6 haloalkynyl group, a phenyl group optionally substituted with 0 to 5 substituents B, or a 5- or 6-membered aromatic heterocyclic group optionally substituted with 0 to 4 substituents B) in R2 in Formula (1-2) has the same meaning as above.

The “Rx3OC(═O)—” is preferably a methoxycarbonyl group, an ethoxycarbonyl group, an isopropyloxycarbonyl group, a difluoromethoxycarbonyl group, a trifluoromethoxycarbonyl group, a cyclopropyloxycarbonyl group, a cyclopentyloxycarbonyl group, an allyloxycarbonyl group, a propargyloxycarbonyl group, a phenyloxycarbonyl group, or a 4-nitrophenyloxycarbonyl group, more preferably a methoxycarbonyl group, an ethoxycarbonyl group, an isopropyloxycarbonyl group, a cyclopropyloxycarbonyl group, a propargyloxycarbonyl group, a phenyloxycarbonyl group, or a 4-nitrophenyloxycarbonyl group, and particularly preferably a methoxycarbonyl group or an ethoxycarbonyl group.

Each term of “Rx4S(O)p-” (where Rx4 represents a hydroxyl group, a halogen atom, a C1 to C6 alkyl group optionally substituted with a substituent A, a C1 to C6 haloalkyl group, a C3 to C8 cycloalkyl group optionally substituted with a substituent B, a C2 to C6 alkenyl group optionally substituted with a substituent A, a C2 to C6 haloalkenyl group, a C2 to C6 alkynyl group optionally substituted with a substituent A, a C2 to C6 haloalkynyl group, or a phenyl group optionally substituted with 0 to 5 substituents B, and p represents an integer of 0, 1, or 2) in R2 in Formula (1-2) has the same meaning as above. The “Rx4S(O)p-” is preferably a methylsulfonyl group, an ethylsulfonyl group, a trifluoromethylsulfonyl group, a 1,1,2,2,2-pentafluoroethylsulfonyl group, a cyclopropylsulfonyl group, a cyclopentylsulfonyl group, a phenylsulfonyl group, a 4-fluorophenylsulfonyl group, a 4-chlorophenylsulfonyl group, a 4-methylphenylsulfonyl group, or a 4-trifluoromethylphenylsulfonyl group, more preferably a methylsulfonyl group, an ethylsulfonyl group, a trifluoromethylsulfonyl group, a 4-methylphenylsulfonyl group, or a 4-trifluoromethylphenylsulfonyl group, and particularly preferably a methylsulfonyl group, an ethylsulfonyl group, or a 4-methylphenylsulfonyl group.

The term “3- to 6-membered ring group containing one or two oxygen atoms” in R2 in Formula (1-2) has the same meaning as above. The “3- to 6-membered ring group containing one or two oxygen atoms” is preferably an oxiran-2-yl group, an oxetan-3-yl group, a tetrahydrofuran-2-yl group, a tetrahydrofuran-3-yl group, a tetrahydro-2H-pyran-2-yl group, a tetrahydro-2H-pyran-3-yl group, a tetrahydro-2H-pyran-4-yl group, a 1,4-dioxan-2-yl group, a 3-dihydrofuran-2(3H)-one group, a 4-dihydrofuran-2(3H)-one group, a 3-tetrahydro-2H-pyran-2-one group, or a 4-tetrahydro-2H-pyran-2-one group, more preferably a tetrahydrofuran-2-yl group, a tetrahydrofuran-3-yl group, a tetrahydro-2H-pyran-2-yl group, a tetrahydro-2H-pyran-3-yl group, a tetrahydro-2H-pyran-4-yl group, a 3-dihydro Furan-2(3H)-one group, or a 4-dihydrofuran-2(3H)-one group, still more preferably a tetrahydro-2H-pyran-2-yl group, a tetrahydro-2H-pyran-4-yl group, or a 3-dihydrofuran-2(3H)-one group, and particularly preferably a tetrahydro-2H-pyran-4-yl group or a 3-dihydrofuran-2(3H)-one group.

In Formula (1-1) or Formula (1-2), R3 represents a hydrogen atom, a C1 to C6 alkyl group optionally substituted with a substituent A, a C1 to C6 haloalkyl group, a C3 to C8 cycloalkyl group optionally substituted with a substituent B, a C2 to C6 alkenyl group optionally substituted with a substituent A, a C2 to C6 haloalkenyl group, a C2 to C6 alkynyl group optionally substituted with a substituent A, a C2 to C6 haloalkynyl group, Rx1Rx2NC(═O)— (where Rx1 and Rx2 each independently represent a hydrogen atom, a hydroxyl group, a cyano group, a C1 to C6 alkyl group optionally substituted with a substituent A, a C1 to C6 haloalkyl group, a C3 to C8 cycloalkyl group optionally substituted with a substituent B, a C2 to C6 alkenyl group optionally substituted with a substituent A, a C2 to C6 haloalkenyl group, a C2 to C6 alkynyl group optionally substituted with a substituent A, a C2 to C6 haloalkynyl group, a C1 to C6 alkoxy group, a C1 to C6 haloalkoxy group, a C3 to C8 cycloalkoxy group, a phenyl group optionally substituted with 0 to 5 substituents B, or a 5- or 6-membered aromatic heterocyclic group optionally substituted with 0 to 4 substituents B, or Rx1 and Rx2 together with a nitrogen atom to which they are bonded form an aziridinyl group, an azetidinyl group, a pyrrolidinyl group, a piperidinyl group, a piperazinyl group, a morpholinyl group, a thiomorpholinyl group, a dioxothiomorpholinyl group, a thiazolidinyl group, an azepanyl group, or an azocanyl group), Rx3C(═O)— (where Rx3 represents a hydrogen atom, a hydroxyl group, a halogen atom, a C1 to C6 alkyl group optionally substituted with a substituent A, a C1 to C6 haloalkyl group, a C3 to C8 cycloalkyl group optionally substituted with a substituent B, a C2 to C6 alkenyl group optionally substituted with a substituent A, a C2 to C6 haloalkenyl group, a C2 to C6 alkynyl group optionally substituted with a substituent A, a C2 to C6 haloalkynyl group, a phenyl group optionally substituted with 0 to 5 substituents B, or a 5- or 6-membered aromatic heterocyclic group optionally substituted with 0 to 4 substituents B), Rx3OC(═O)— (where Rx3 has the same meaning as above), or Rx4S(O)p- (where Rx4 represents a hydroxyl group, a halogen atom, a C1 to C6 alkyl group optionally substituted with a substituent A, a C1 to C6 haloalkyl group, a C3 to C8 cycloalkyl group optionally substituted with a substituent B, a C2 to C6 alkenyl group optionally substituted with a substituent A, a C2 to C6 haloalkenyl group, a C2 to C6 alkynyl group optionally substituted with a substituent A, a C2 to C6 haloalkynyl group, or a phenyl group optionally substituted with 0 to 5 substituents B, and p represents an integer of 0, 1, or 2).

Among them, R3 is preferably a hydrogen atom, a C1 to C6 alkyl group optionally substituted with a substituent A, a C1 to C6 haloalkyl group, a C3 to C8 cycloalkyl group optionally substituted with a substituent B, a C2 to C6 alkenyl group optionally substituted with a substituent A, a C2 to C6 alkynyl group optionally substituted with a substituent A, Rx1Rx2NC(═O)— (where Rx1 and Rx2 have the same meanings as above), Rx3C(═O)— (where Rx3 has the same meaning as above), or Rx3OC(═O)— (where Rx3 has the same meaning as above).

Furthermore, R3 is preferably a hydrogen atom, a C1 to C6 alkyl group optionally substituted with a substituent A, a C1 to C6 haloalkyl group, a C3 to C8 cycloalkyl group optionally substituted with a substituent B, Rx1Rx2NC(═O)— (where Rx1 and Rx2 have the same meanings as above), Rx3C(═O)— (where Rx3 has the same meaning as above), or Rx3OC(═O)— (where Rx3 has the same meaning as above).

In particular, R3 is preferably a hydrogen atom, a C1 to C6 alkyl group optionally substituted with a substituent A, a C1 to C6 haloalkyl group, or Rx3C(═O)— (where Rx3 has the same meaning as above).

R3 in Formula (1-1) or Formula (1-2) contains a hydrogen atom.

The C1 to C6 alkyl group of the “C1 to C6 alkyl group optionally substituted with a substituent A” in R3 in Formula (1-1) or Formula (1-2) has the same meaning as above, and is preferably a methyl group, an ethyl group, a propyl group, an isopropyl group, a butyl group, or an isobutyl group, more preferably a methyl group, an ethyl group, a propyl group, or an isopropyl group, still more preferably a methyl group, an ethyl group, or a propyl group, and particularly preferably a methyl group. When having a substituent A, one hydrogen atom in the C1 to C6 alkyl group is substituted with the substituent A.

The “C1 to C6 haloalkyl group” in R3 in Formula (1-1) or Formula (1-2) has the same meaning as above, and is preferably a difluoromethyl group, a trifluoromethyl group, a 2,2-difluoroethyl group, a 2,2,2-trifluoroethyl group, a 1,2,2,2-tetrafluoroethyl group, a 1,1,2,2,2-pentafluoroethyl group, or a 1,1,2,2,3,3,3-heptafluoropropyl group, more preferably a difluoromethyl group, a trifluoromethyl group, a 2,2-difluoroethyl group, a 2,2,2-trifluoroethyl group, a 1,2,2,2-tetrafluoroethyl group, or a 1,1,2,2,3,3,3-heptafluoropropyl group, still more preferably a difluoromethyl group, a trifluoromethyl group, a 2,2-difluoroethyl group, or a 2,2,2-trifluoroethyl group, and particularly preferably a 2,2,2-trifluoroethyl group.

The C3 to C8 cycloalkyl group of the “C3 to C8 cycloalkyl group optionally substituted with a substituent B” in R3 in Formula (1-1) or Formula (1-2) has the same meaning as above, and is preferably a cyclopropyl group, a cyclobutyl group, a cyclopentyl group, a cyclohexyl group, or a cycloheptyl group, more preferably a cyclopropyl group, a cyclobutyl group, a cyclopentyl group, or a cyclohexyl group, and particularly preferably a cyclopropyl group or a cyclobutyl group. When having the substituent described in the substituent A, a hydrogen atom in the C3 to C8 cycloalkyl group is optionally substituted with the substituent described in the substituent A.

The C2 to C6 alkenyl group of the “C2 to C6 alkenyl group optionally substituted with a substituent A” in R3 in Formula (1-1) or Formula (1-2) has the same meaning as above, and is preferably a vinyl group, a 1-propenyl group, an allyl group, a 2-methylallyl group, a 1-butenyl group, a 2-butenyl group, or a 3-butenyl group, more preferably a vinyl group, a 1-propenyl group, an allyl group, a 2-methylallyl group, or a 3-butenyl group, and particularly preferably an allyl group, a 2-methylallyl group, or a 3-butenyl group. When having the substituent described in the substituent A, a hydrogen atom in the C2 to C6 alkenyl group is optionally substituted with the substituent described in the substituent A.

The “C2 to C6 haloalkenyl group” in R3 in Formula (1-1) or Formula (1-2) has the same meaning as above, is preferably a 2-fluorovinyl group, a 2,2-difluorovinyl group, a 3-fluoroallyl group, or a 3,3-difluoroallyl group, more preferably a 3-fluoroallyl group or a 3,3-difluoroallyl group, and particularly preferably a 3,3-difluoroallyl group.

The C2 to C6 alkynyl group of the “C2 to C6 alkynyl group optionally substituted with a substituent A” in R3 in Formula (1-1) or Formula (1-2) has the same meaning as above, and is preferably an ethynyl group, a 1-propynyl group, a propargyl group, a 1-butynyl group, a 2-butynyl group, or a 3-butynyl group, more preferably an ethynyl group, a 1-propynyl group, a propargyl group, or a 2-butynyl group, and particularly preferably a propargyl group or a 2-butynyl group. When having a substituent A, one hydrogen atom in the C2 to C6 alkynyl group is substituted with the substituent A.

The “C2 to C6 haloalkynyl group” in R3 in Formula (1-1) or Formula (1-2) has the same meaning as above, and is preferably a 3,3-difluoro-1-propynyl group, a 3,3,3-trifluoro-1-propynyl group, a 4,4-difluoro-1-butynyl group, a 4,4-difluoro-2-butynyl group, a 4,4,4-trifluoro-1-butynyl group, or a 4,4,4-trifluoro-2-butynyl group, more preferably a 3,3-difluoro-1-propynyl group, a 3,3,3-trifluoro-1-propynyl group, or a 4,4,4-trifluoro-1-butynyl group, and particularly preferably a 3,3-difluoro-1-propynyl group or a 3,3,3-trifluoro-1-propynyl group.

Each term of Rx1Rx2NC(═O)— (where Rx1 and Rx2 each independently represent a hydrogen atom, a hydroxyl group, a cyano group, a C1 to C6 alkyl group optionally substituted with a substituent A, a C1 to C6 haloalkyl group, a C3 to C8 cycloalkyl group optionally substituted with a substituent B, a C2 to C6 alkenyl group optionally substituted with a substituent A, a C2 to C6 haloalkenyl group, a C2 to C6 alkynyl group optionally substituted with a substituent A, a C2 to C6 haloalkynyl group, a C1 to C6 alkoxy group, a C1 to C6 haloalkoxy group, a C3 to C8 cycloalkoxy group, a phenyl group optionally substituted with 0 to 5 substituents B, or a 5- or 6-membered aromatic heterocyclic group optionally substituted with 0 to 4 substituents B, or Rx1 and Rx2 together with a nitrogen atom to which they are bonded form an aziridinyl group, an azetidinyl group, a pyrrolidinyl group, a piperidinyl group, a piperazinyl group, a morpholinyl group, a thiomorpholinyl group, a dioxothiomorpholinyl group, a thiazolidinyl group, an azepanyl group, or an azocanyl group) in R3 in Formula (1-1) or Formula (1-2) has the same meaning as above.

The “Rx1Rx2NC(═O)—” is preferably a carbamoyl group, a methylcarbamoyl group, an ethylcarbamoyl group, a propylcarbamoyl group, an isopropylcarbamoyl group, a butylcarbamoyl group, an isobutylcarbamoyl group, a sec-butylcarbamoyl group, a t-butylcarbamoyl group, a pentylcarbamoyl group, an isopentylcarbamoyl group, a 2-methylbutylcarbamoyl group, a neopentylcarbamoyl group, a 1-ethylpropylcarbamoyl group, a hexylcarbamoyl group, a methoxymethylcarbamoyl group, an ethoxymethylcarbamoyl group, a propyloxymethylcarbamoyl group, an isopropyloxymethylcarbamoyl group, a 2-methoxyethylcarbamoyl group, a 2-ethoxyethylcarbamoyl group, a cyanomethylcarbamoyl group, a 1-cyanoethylcarbamoyl group, a 2-cyanoethylcarbamoyl group, a cyclopropylmethylcarbamoyl group, a cyclobutylmethylcarbamoyl group, a benzylcarbamoyl group, a 1-phenethylcarbamoyl group, a 1-difluoroethylcarbamoyl group, a 2,2-difluoroethylcarbamoyl group, a 2,2,2-trifluoroethylearbamoyl group, a 2,2,3,3,3-pentafluoropropylcarbamoyl group, a 2,2,3,3,4,4,4-heptafluorobutylcarbamoyl group, a 3,3,4,4,5,5,5-heptafluoropentylcarbamoyl group, a cyclopropylcarbamoyl group, a 1-cyanocyclopropylcarbamoyl group, an allylcarbamoyl group, a 3,3-difluoroallylcarbamoyl group, a propargylcarbamoyl group, a methoxycarbamoyl group, a trifluoroethoxycarbamoyl group, a phenylcarbamoyl group, a 4-cyanophenylcarbamoyl group, a 2-fluorophenylcarbamoyl group, a 3-fluorophenylcarbamoyl group, a 4-fluorophenylcarbamoyl group, a 2-chlorophenylcarbamoyl group, a 3-chlorophenylcarbamoyl group, a 4-chlorophenylcarbamoyl group, a 2-trifluoromethylphenylcarbamoyl group, a 3-trifluoromethylphenylcarbamoyl group, a 4-trifluoromethylphenylcarbamoyl group, a 2-methoxyphenylcarbamoyl group, a 3-methoxyphenylcarbamoyl group, a 4-methoxyphenylcarbamoyl group, a pyrrolidinecarbonyl group, a piperidinecarbonyl group, a piperazinecarbonyl group, a morpholinecarbonyl group, a thiomorpholinecarbonyl group, a dioxothiomorpholinecarbonyl group, a thiazolidinecarbonyl group, an azepanecarbonyl group, an azocancarbonyl group, a dimethylcarbamoyl group, an ethylmethylcarbamoyl group, a diethylcarbamoyl group, a methylpropylcarbamoyl group, an ethylpropylcarbamoyl group, an isopropylmethylcarbamoyl group, an isopropylethylcarbamoyl group, a diisopropylcarbamoyl group, a butylmethylcarbamoyl group, a butylethylcarbamoyl group, a sec-butylmethylcarbamoyl group, a sec-butylethylcarbamoyl group, an isobutylmethylcarbamoyl group, an isobutylethyl carbamoyl group, a t-butylmethylcarbamoyl group, a t-butylethylcarbamoyl group, a methylpentylcarbamoyl group, an ethylpentylcarbamoyl group, an isopentylmethylcarbamoyl group, an isopentylethylcarbamoyl group, a methyl(2-methylbutyl)carbamoyl group, an ethyl(2-methylbutyl)carbamoyl group, a hexylmethylcarbamoyl group, a hexylethylcarbamoyl group, a (methoxymethyl)methylcarbamoyl group, an (ethoxymethyl)methylcarbamoyl group, a (methoxyethyl)methylcarbamoyl group, an (ethoxyethyl)methylcarbamoyl group, a (propyloxymethyl)methylcarbamoyl group, an (isopropyloxymethyl)methylcarbamoyl group, a (methoxymethyl)ethylcarbamoyl group, an (ethoxymethyl)ethylcarbamoyl group, a (cyanomethyl)methylcarbamoyl group, a (cyanomethyl)ethylcarbamoyl group, a (cyclopropylmethyl)methylcarbamoyl group, a (cyclopropylmethyl)ethylcarbamoyl group, a (benzyl)methylcarbamoyl group, a methyl(pyridin-3-ylmethyl)carbamoyl group, a methyl(2,2,2-trifluoroethyl)carbamoyl group, a methyl(2,2,3,3,3-pentafluoropropyl)carbamoyl group, a methyl(2,2,3,3,4,4,4-heptafluorobutyl)carbamoyl group, a methyl(3,3,4,4,5,5,5-heptafluoropentyl)carbamoyl group, a (cyclopropyl)methylcarbamoyl group, a (cyclopropyl)ethylcarbamoyl group, a (1-cyanocyclopropyl)methylcarbamoyl group, a (1-cyanocyclopropyl)ethylcarbamoyl group, a (cyclobutyl)methylcarbamoyl group, a (cyclopentyl)methylcarbamoyl group, a (cyclohexyl)methylcarbamoyl group, an (allyl)methylcarbamoyl group, a methyl(2-propyn-1-yl)carbamoyl group, an ethyl(2-propyn-1-yl)carbamoyl group, a (methoxy) methylcarbamoyl group, an (ethoxy)methylcarbamoyl group, a (trifluoroethoxy)methylcarbamoyl group, a methylphenylcarbamoyl group, a diphenylcarbamoyl group, a methyl(pyridin-2-yl)carbamoyl group, a methyl(6-methyl-pyridin-2-yl)carbamoyl group, a methyl(pyridin-3-yl)carbamoyl group, or a methyl(pyridin-4-yl)carbamoyl group, more preferably a carbamoyl group, a methylcarbamoyl group, an ethylcarbamoyl group, a pyrrolidinecarbonyl group, a piperidinecarbonyl group, a 4-cyanopiperidinecarbonyl group, a 4-fluoropiperidinecarbonyl group, a 4,4-difluoropiperidinecarbonyl group, a 4-methylpiperidinecarbonyl group, a 4-trifluoromethylpiperidinecarbonyl group, a 4-methoxycarbonylpiperidinecarbonyl group, a piperazinecarbonyl group, a morpholinecarbonyl group, a thiomorpholinecarbonyl group, a dioxothiomorpholinecarbonyl group, a thiazolidinecarbonyl group, an azepanecarbonyl group, an azocancarbonyl group, a dimethylcarbamoyl group, an ethylmethylcarbamoyl group, a diethylcarbamoyl group, a methylpropylcarbamoyl group, an ethylpropylcarbamoyl group, an isopropylmethylcarbamoyl group, an isopropylethylcarbamoyl group, a diisopropylcarbamoyl group, a butylmethylcarbamoyl group, a butylethylcarbamoyl group, a sec-butylmethylcarbamoyl group, a sec-butylethylcarbamoyl group, an isobutylmethylcarbamoyl group, an isobutylethylcarbamoyl group, a t-butylmethylcarbamoyl group, a t-butylethylcarbamoyl group, a methylpentylcarbamoyl group, an ethylpentylcarbamoyl group, an isopentylmethylcarbamoyl group, an isopentylethylcarbamoyl group, a methyl(2-methylbutyl)carbamoyl group, an ethyl(2-methylbutyl)carbamoyl group, a hexylmethylcarbamoyl group, a hexylethylcarbamoyl group, a (methoxymethyl)methylcarbamoyl group, an (ethoxymethyl)methylcarbamoyl group, a (methoxyethyl)methylcarbamoyl group, an (ethoxyethyl)methylcarbamoyl group, a (propyloxymethyl)methylcarbamoyl group, an (isopropyloxymethyl)methylcarbamoyl group, a (methoxymethyl)ethylcarbamoyl group, an (ethoxymethyl)ethylcarbamoyl group, a (cyanomethyl)methylcarbamoyl group, a (cyanomethyl)ethylcarbamoyl group, a (cyclopropylmethyl)methylcarbamoyl group, a (cyclopropylmethyl)ethylcarbamoyl group, a (benzyl)methylcarbamoyl group, a methyl(pyridin-3-ylmethyl)carbamoyl group, a methyl(2,2,2-trifluoroethyl)carbamoyl group, a methyl(2,2,3,3,3-pentafluoropropyl)carbamoyl group, a methyl(2,2,3,3,4,4,4-heptafluorobutyl)carbamoyl group, a methyl(3,3,4,4,5,5,5-heptafluoropentyl)carbamoyl group, a (cyclopropyl)methylcarbamoyl group, a (cyclopropyl)ethylcarbamoyl group, a (1-cyanocyclopropyl)methylcarbamoyl group, a (1-cyanocyclopropyl)ethylcarbamoyl group, a (cyclobutyl)methylcarbamoyl group, a (cyclopentyl)methylcarbamoyl group, a (cyclohexyl)methylcarbamoyl group, an (allyl)methylcarbamoyl group, a methyl(2-propyn-1-yl)carbamoyl group, an ethyl(2-propyn-1-yl)carbamoyl group, a (methoxy)methylcarbamoyl group, an (ethoxy)methylcarbamoyl group, a (trifluoroethoxy)methylcarbamoyl group, a methylphenylcarbamoyl group, a diphenylcarbamoyl group, a methyl(pyridin-2-yl)carbamoyl group, a methyl(6-methyl-pyridin-2-yl)carbamoyl group, a methyl(pyridin-3-yl)carbamoyl group, or a methyl(pyridin-4-yl)carbamoyl group, and
particularly preferably a carbamoyl group, a methylcarbamoyl group, a pyrrolidinecarbonyl group, a piperidinecarbonyl group, a dimethylcarbamoyl group, or an ethylmethylcarbamoyl group.

Each term of “Rx3C(═O)—” (where Rx3 represents a hydrogen atom, a hydroxyl group, a halogen atom, a C1 to C6 alkyl group optionally substituted with a substituent A, a C1 to C6 haloalkyl group, a C3 to C8 cycloalkyl group optionally substituted with a substituent B, a C2 to C6 alkenyl group optionally substituted with a substituent A, a C2 to C6 haloalkenyl group, a C2 to C6 alkynyl group optionally substituted with a substituent A, a C2 to C6 haloalkynyl group, a phenyl group optionally substituted with 0 to 5 substituents B, or a 5- or 6-membered aromatic heterocyclic group optionally substituted with 0 to 4 substituents B) in R3 in Formula (1-1) or Formula (1-2) has the same meaning as above.

The “Rx3C(═O)—” preferably a formyl group, an acetyl group, a propionyl group, an isopropionyl group, a butyryl group, an isobutyryl group, a t-butyryl group, a pentanoyl group, a hexanoyl group, a (1H-pyrazol-1-yl)acetyl group, a (1H-1,2,4-triazol-1-yl)acetyl group, a 2-fluoroacetyl group, a 2-chloroacetyl group, a 2-bromoacetyl group, a 2,2-difluoroacetyl group, a 2,2-dichloroacetyl group, a 2,2-dibromoacetyl group, a 2,2,2-trifluoroacetyl group, a 2-chloro-2,2-difluoroacetyl group, a 2,2,2-trichloroacetyl group, a 2,2,3,3-tetrafluoropropionyl group, a 2,2,3,3,3-pentafluoropropionyl group, a 2,2-difluoropropionyl group, a 3,3,3-trifluoropropionyl group, a 2,2,3,3,4,4-hexafluorobutyryl group, a 2,2,3,3,4,4,4-heptafluorobutyryl group, a 2,2,3,3,4,4,5,5-octafluoropentanoyl group, a 2,2,3,3,4,4,5,5,5-nonafluoropentanoyl group, a 2,2,3,3,4,4,5,5,6,6-decafluorohexanoyl group, a 2,2,3,3,4,4,5,5,6,6,6-undecafluorohexanoyl group, a cyclopropylcarbonyl group, a 1-fluorocyclopropyl-1-carbonyl group, a 1-trifluoromethylcyclopropyl-1-carbonyl group, a 1-cyanocyclopropyl-1-carbonyl group, a 1-methylcyclopropyl-1-carbonyl group, a 2,2,3,3-tetramethyl-1-cyclopropylcarbonyl group, a cyclobutylcarbonyl group, a 1-trifluoromethylcyclobutyl-1-carbonyl group, a cyclopentylcarbonyl group, a cyclohexylcarbonyl group, a benzoyl group, a 2-fluorobenzoyl group, a 3-fluorobenzoyl group, a 4-fluorobenzoyl group, a 2-chlorobenzoyl group, a 3-chlorobenzoyl group, a 4-chlorobenzoyl group, a 2-bromobenzoyl group, a 3-bromobenzoyl group, a 4-bromobenzoyl group, a 2-methylbenzoyl group, a 3-methylbenzoyl group, a 4-methylbenzoyl group, a 2-trifluoromethylbenzoyl group, a 3-trifluoromethylbenzoyl group, a 4-trifluoromethylbenzoyl group, a 2-trifluoromethoxybenzoyl group, a 3-trifluoromethoxybenzoyl group, a 4-trifluoromethoxybenzoyl group, a picolinoyl group, a nicotinoyl group, or an isonicotinoyl group,
more preferably an acetyl group, a propionyl group, an isopropionyl group, a butyryl group, an isobutyryl group, a t-butyryl group, a pentanoyl group, a hexanoyl group, a 2-(1H-pyrazol-1-yl)acetyl group, a 2-(1H-1,2,4-triazol-1-yl)acetyl group, a 2-chloroacetyl group, a 2,2-difluoroacetyl group, a 2,2-dichloroacetyl group, a 2,2-dibromoacetyl group, a 2,2,2-trifluoroacetyl group, a 2-chloro-2,2-difluoroacetyl group, a 2,2,2-trichloroacetyl group, a 2,2,3,3-tetrafluoropropionyl group, a 2,2,3,3,3-pentafluoropropionyl group, a 2,2-difluoropropionyl group, a 3,3,3-trifluoropropionyl group, a 2,2,3,3,4,4-hexafluorobutyryl group, a 2,2,3,3,4,4,4-heptafluorobutyryl group, a 2,2,3,3,4,4,5,5-octafluoropentanoyl group, a 2,2,3,3,4,4,5,5,5-nonafluoropentanoyl group, a 2,2,3,3,4,4,5,5,6,6-decafluorohexanoyl group, a 2,2,3,3,4,4,5,5,6,6,6-undecafluorohexanoyl group, a cyclopropylcarbonyl group, a 1-fluorocyclopropyl-1-carbonyl group, a 1-trifluoromethylcyclopropyl-1-carbonyl group, a 1-cyanocyclopropyl-1-carbonyl group, a 1-methylcyclopropyl-1-carbonyl group, a 2,2,3,3-tetramethyl-1-cyclopropylcarbonyl group, a cyclobutylcarbonyl group, a 1-trifluoromethylcyclobutyl-1-carbonyl group, a cyclopentylcarbonyl group, a cyclohexylcarbonyl group, a benzoyl group, a 2-fluorobenzoyl group, a 3-fluorobenzoyl group, a 4-fluorobenzoyl group, a 2-chlorobenzoyl group, a 3-chlorobenzoyl group, a 4-chlorobenzoyl group, a 2-trifluoromethylbenzoyl group, a 3-trifluoromethylbenzoyl group, a 4-trifluoromethylbenzoyl group, a 2-trifluoromethoxybenzoyl group, a 3-trifluoromethoxybenzoyl group, a 4-trifluoromethoxybenzoyl group, a picolinoyl group, a nicotinoyl group, or an isonicotinoyl group,
still more preferably an acetyl group, a propionyl group, an isopropionyl group, a butyryl group, an isobutyryl group, a t-butyryl group, a pentanoyl group, a hexanoyl group, a 2-(1H-pyrazol-1-yl)acetyl group, a 2-(1H-1,2,4-triazol-1-yl)acetyl group, a 2-chloroacetyl group, a 2,2-difluoroacetyl group, a 2,2-dichloroacetyl group, a 2,2-dibromoacetyl group, a 2,2,2-trifluoroacetyl group, a 2-chloro-2,2-difluoroacetyl group, a 2,2,2-trichloroacetyl group, a 2,2,3,3-tetrafluoropropionyl group, a 2,2,3,3,3-pentafluoropropionyl group, a 2,2-difluoropropionyl group, a 3,3,3-trifluoropropionyl group, a 2,2,3,3,4,4-hexafluorobutyryl group, a 2,2,3,3,4,4,4-heptafluorobutyryl group, a 2,2,3,3,4,4,5,5-octafluoropentanoyl group, a 2,2,3,3,4,4,5,5,5-nonafluoropentanoyl group, a 2,2,3,3,4,4,5,5,6,6-decafluorohexanoyl group, a 2,2,3,3,4,4,5,5,6,6,6-undecafluorohexanoyl group, a cyclohexylcarbonyl group, a benzoyl group, a 4-trifluoromethoxybenzoyl group, a picolinoyl group, or a nicotinoyl group, and
particularly preferably an acetyl group, a cyclohexylcarbonyl group, or a 4-trifluoromethoxybenzoyl group.

Each term of “Rx3OC(═O)—” (where Rx3 represents a hydrogen atom, a hydroxyl group, a halogen atom, a C1 to C6 alkyl group optionally substituted with a substituent A, a C1 to C6 haloalkyl group, a C3 to C8 cycloalkyl group optionally substituted with a substituent B, a C2 to C6 alkenyl group optionally substituted with a substituent A, a C2 to C6 haloalkenyl group, a C2 to C6 alkynyl group optionally substituted with a substituent A, a C2 to C6 haloalkynyl group, a phenyl group optionally substituted with 0 to 5 substituents B, or a 5- or 6-membered aromatic heterocyclic group optionally substituted with 0 to 4 substituents B) in R3 in Formula (1-1) or Formula (1-2) has the same meaning as above.

The “Rx3OC(═O)—” is preferably a methoxycarbonyl group, an ethoxycarbonyl group, a propyloxycarbonyl group, an isopropyloxycarbonyl group, a t-butyloxycarbonyl group, a difluoromethoxycarbonyl group, a trifluoromethoxycarbonyl group, a cyclopropyloxycarbonyl group, a cyclopentyloxycarbonyl group, an allyloxycarbonyl group, a propargyloxycarbonyl group, a phenyloxycarbonyl group, or a 4-nitrophenyloxycarbonyl group, more preferably a methoxycarbonyl group, an ethoxycarbonyl group, a propyloxycarbonyl group, an isopropyloxycarbonyl group, a t-butyloxycarbonyl group, a cyclopropyloxycarbonyl group, a propargyloxycarbonyl group, a phenyloxycarbonyl group, or a 4-nitrophenyloxycarbonyl group, and particularly preferably a methoxycarbonyl group, an ethoxycarbonyl group, a propyloxycarbonyl group, or a t-butyloxycarbonyl group.

Each term of “Rx4S(O)p-” (where Rx4 represents a hydroxyl group, a halogen atom, a C1 to C6 alkyl group optionally substituted with a substituent A, a C1 to C6 haloalkyl group, a C3 to C8 cycloalkyl group optionally substituted with a substituent B, a C2 to C6 alkenyl group optionally substituted with a substituent A, a C2 to C6 haloalkenyl group, a C2 to C6 alkynyl group optionally substituted with a substituent A, a C2 to C6 haloalkynyl group, or a phenyl group optionally substituted with 0 to 5 substituents B, and p represents an integer of 0, 1, or 2) in R3 in Formula (1-1) or Formula (1-2) has the same meaning as above.

The “Rx4S(O)p-” is preferably a methylsulfonyl group, an ethylsulfonyl group, a trifluoromethylsulfonyl group, a 1,1,2,2,2-pentafluoroethylsulfonyl group, a cyclopropylsulfonyl group, a cyclopentylsulfonyl group, a phenylsulfonyl group, a 4-fluorophenylsulfonyl group, a 4-chlorophenylsulfonyl group, a 4-methylphenylsulfonyl group, or a 4-trifluoromethylphenylsulfonyl group, more preferably a methylsulfonyl group, an ethylsulfonyl group, a trifluoromethylsulfonyl group, a 4-methylphenylsulfonyl group, or a 4-trifluoromethylphenylsulfonyl group, and particularly preferably a methylsulfonyl group, an ethylsulfonyl group, or a 4-methylphenylsulfonyl group.

In Formula (1-1) or Formula (1-2), R4 represents a hydrogen atom, a C1 to C6 alkyl group optionally substituted with a substituent A, a C1 to C6 haloalkyl group, a C3 to C8 cycloalkyl group optionally substituted with a substituent B, a C2 to C6 alkenyl group optionally substituted with a substituent A, a C2 to C6 haloalkenyl group, a C2 to C6 alkynyl group optionally substituted with a substituent A, a C2 to C6 haloalkynyl group, Rx1Rx2NC(═O)— (where Rx1 and Rx2 each independently represent a hydrogen atom, a hydroxyl group, a cyano group, a C1 to C6 alkyl group optionally substituted with a substituent A, a C1 to C6 haloalkyl group, a C3 to C8 cycloalkyl group optionally substituted with a substituent B, a C2 to C6 alkenyl group optionally substituted with a substituent A, a C2 to C6 haloalkenyl group, a C2 to C6 alkynyl group optionally substituted with a substituent A, a C2 to C6 haloalkynyl group, a C1 to C6 alkoxy group, a C1 to C6 haloalkoxy group, a C3 to C8 cycloalkoxy group, a phenyl group optionally substituted with 0 to 5 substituents B, or a 5- or 6-membered aromatic heterocyclic group optionally substituted with 0 to 4 substituents B, or Rx1 and Rx2 together with a nitrogen atom to which they are bonded form an aziridinyl group, an azetidinyl group, a pyrrolidinyl group, a piperidinyl group, a piperazinyl group, a morpholinyl group, a thiomorpholinyl group, a dioxothiomorpholinyl group, a thiazolidinyl group, an azepanyl group, or an azocanyl group), Rx3C(═O)— (where Rx3 represents a hydrogen atom, a hydroxyl group, a halogen atom, a C1 to C6 alkyl group optionally substituted with a substituent A, a C1 to C6 haloalkyl group, a C3 to C8 cycloalkyl group optionally substituted with a substituent B, a C2 to C6 alkenyl group optionally substituted with a substituent A, a C2 to C6 haloalkenyl group, a C2 to C6 alkynyl group optionally substituted with a substituent A, a C2 to C6 haloalkynyl group, a phenyl group optionally substituted with 0 to 5 substituents B, or a 5- or 6-membered aromatic heterocyclic group optionally substituted with 0 to 4 substituents B), Rx3OC(═O)— (where Rx3 has the same meaning as above), or Rx4S(O)p- (where Rx4 represents a hydroxyl group, a halogen atom, a C1 to C6 alkyl group optionally substituted with a substituent A, a C1 to C6 haloalkyl group, a C3 to C8 cycloalkyl group optionally substituted with a substituent B, a C2 to C6 alkenyl group optionally substituted with a substituent A, a C2 to C6 haloalkenyl group, a C2 to C6 alkynyl group optionally substituted with a substituent A, a C2 to C6 haloalkynyl group, or a phenyl group optionally substituted with 0 to 5 substituents B, and p represents an integer of 0, 1, or 2).

Among them, R4 is preferably a hydrogen atom, a C1 to C6 alkyl group optionally substituted with a substituent A, a C1 to C6 haloalkyl group, a C3 to C8 cycloalkyl group optionally substituted with a substituent B, a C2 to C6 alkenyl group optionally substituted with a substituent A, a C2 to C6 alkynyl group optionally substituted with a substituent A, Rx1Rx2NC(═O)— (where Rx1 and Rx2 have the same meanings as above), Rx3C(═O)— (where Rx3 has the same meaning as above), Rx3OC(═O)— (where Rx3 has the same meaning as above), or Rx4S(O)p- (where Rx4 and p have the same meanings as above).

Furthermore, R4 is preferably a hydrogen atom, a C1 to C6 alkyl group optionally substituted with a substituent A,
a C1 to C6 haloalkyl group, a C3 to C8 cycloalkyl group optionally substituted with a substituent B, Rx1Rx2NC(═O)— (where Rx1 and Rx2 have the same meanings as above), Rx3C(═O)— (where Rx3 has the same meaning as above), Rx3OC(═O)— (where Rx3 has the same meaning as above), or Rx4S(O)p- (where Rx4 and p have the same meanings as above).

Particularly, R4 is preferably a hydrogen atom, a C1 to C6 alkyl group optionally substituted with a substituent A, Rx1Rx2NC(═O)— (where Rx1 and Rx2 have the same meanings as above), Rx3C(═O)— (where Rx3 has the same meaning as above), Rx3OC(═O)— (where Rx3 has the same meaning as above), or Rx4S(O)p- (where Rx4 and p have the same meanings as above).

R4 in Formula (1-1) or Formula (1-2) contains a hydrogen atom.

The C1 to C6 alkyl group of the “C1 to C6 alkyl group optionally substituted with a substituent A” in R4 in Formula (1-1) or Formula (1-2) has the same meaning as above, and is preferably a methyl group, an ethyl group, a propyl group, an isopropyl group, a butyl group, or an isobutyl group, more preferably a methyl group, an ethyl group, or an isopropyl group, and particularly preferably a methyl group. When having a substituent A, one hydrogen atom in the C1 to C6 alkyl group is substituted with the substituent A.

The “C1 to C6 haloalkyl group” in R4 in Formula (1-1) or Formula (1-2) has the same meaning as above, and is preferably a difluoromethyl group, a trifluoromethyl group, a 2,2-difluoroethyl group, a 2,2,2-trifluoroethyl group, a 1,2,2,2-tetrafluoroethyl group, a 1,1,2,2,2-pentafluoroethyl group, or a 1,1,2,2,3,3,3-heptafluoropropyl group, more preferably a difluoromethyl group, a trifluoromethyl group, a 2,2-difluoroethyl group, or a 2,2,2-trifluoroethyl group, and particularly preferably a 2,2-difluoroethyl group or a 2,2,2-trifluoroethyl group.

The C3 to C8 cycloalkyl group of the “C3 to C8 cycloalkyl group optionally substituted with a substituent B” in R4 in Formula (1-1) or Formula (1-2) has the same meaning as above, and is preferably a cyclopropyl group, a cyclobutyl group, a cyclopentyl group, a cyclohexyl group, or a cycloheptyl group, more preferably a cyclopropyl group, a cyclobutyl group, a cyclopentyl group, or a cyclohexyl group, and particularly preferably a cyclopropyl group or a cyclobutyl group. When having the substituent described in the substituent A, a hydrogen atom in the C3 to C8 cycloalkyl group is optionally substituted with the substituent described in the substituent A.

The C2 to C6 alkenyl group of the “C2 to C6 alkenyl group optionally substituted with a substituent A” in R4 in Formula (1-1) or Formula (1-2) has the same meaning as above, and is preferably a vinyl group, a 1-propenyl group, an allyl group, a 2-methylallyl group, a 1-butenyl group, a 2-butenyl group, or a 3-butenyl group, more preferably a vinyl group, a 1-propenyl group, an allyl group, a 2-methylallyl group, or a 3-butenyl group, and particularly preferably an allyl group, a 2-methylallyl group, or a 3-butenyl group. When having the substituent described in the substituent A, a hydrogen atom in the C2 to C6 alkenyl group is optionally substituted with the substituent described in the substituent A.

The “C2 to C6 haloalkenyl group” in R4 in Formula (1-1) or Formula (1-2) has the same meaning as above, and is preferably a 2-fluorovinyl group, a 2,2-difluorovinyl group, a 3-fluoroallyl group, or a 3,3-difluoroallyl group, more preferably a 3-fluoroallyl group or a 3,3-difluoroallyl group, and particularly preferably a 3,3-difluoroallyl group.

The C2 to C6 alkynyl group of the “C2 to C6 alkynyl group optionally substituted with a substituent A” in R4 in Formula (1-1) or Formula (1-2) has the same meaning as above, and is preferably an ethynyl group, a 1-propynyl group, a propargyl group, a 1-butynyl group, a 2-butynyl group, or a 3-butynyl group, more preferably an ethynyl group, a 1-propynyl group, a propargyl group, or a 2-butynyl group, and particularly preferably a propargyl group or a 2-butynyl group. When having a substituent A, one hydrogen atom in the C2 to C6 alkynyl group is substituted with the substituent A.

The “C2 to C6 haloalkynyl group” in R4 in Formula (1-1) or Formula (1-2) has the same meaning as above, and is preferably a 3,3-difluoro-1-propynyl group, a 3,3,3-trifluoro-1-propynyl group, a 4,4-difluoro-1-butynyl group, a 4,4-difluoro-2-butynyl group, a 4,4,4-trifluoro-1-butynyl group, or a 4,4,4-trifluoro-2-butynyl group, more preferably a 3,3-difluoro-1-propynyl group, a 3,3,3-trifluoro-1-propynyl group, or a 4,4,4-trifluoro-1-butynyl group, and particularly preferably a 3,3-difluoro-1-propynyl group or a 3,3,3-trifluoro-1-propynyl group.

Each term of Rx1Rx2NC(═O)— (where Rx1 and Rx2 each independently represent a hydrogen atom, a hydroxyl group, a cyano group, a C1 to C6 alkyl group optionally substituted with a substituent A, a C1 to C6 haloalkyl group, a C3 to C8 cycloalkyl group optionally substituted with a substituent B, a C2 to C6 alkenyl group optionally substituted with a substituent A, a C2 to C6 haloalkenyl group, a C2 to C6 alkynyl group optionally substituted with a substituent A, a C2 to C6 haloalkynyl group, a C1 to C6 alkoxy group, a C1 to C6 haloalkoxy group, a C3 to C8 cycloalkoxy group, a phenyl group optionally substituted with 0 to 5 substituents B, or a 5- or 6-membered aromatic heterocyclic group optionally substituted with 0 to 4 substituents B, or Rx1 and Rx2 together with a nitrogen atom to which they are bonded form an aziridinyl group, an azetidinyl group, a pyrrolidinyl group, a piperidinyl group, a piperazinyl group, a morpholinyl group, a thiomorpholinyl group, a dioxothiomorpholinyl group, a thiazolidinyl group, an azepanyl group, or an azocanyl group) in R4 in Formula (1-1) or Formula (1-2) has the same meaning as above.

The “Rx1Rx2NC(═O)—” is preferably a carbamoyl group, a methylcarbamoyl group, an ethylcarbamoyl group, a propylcarbamoyl group, an isopropylcarbamoyl group, a butylcarbamoyl group, an isobutylcarbamoyl group, a sec-butylcarbamoyl group, a t-butylcarbamoyl group, a pentylcarbamoyl group, an isopentylcarbamoyl group, a 2-methylbutylcarbamoyl group, a neopentylcarbamoyl group, a 1-ethylpropylcarbamoyl group, a hexylcarbamoyl group, a methoxymethylcarbamoyl group, an ethoxymethylcarbamoyl group, a propyloxymethylcarbamoyl group, an isopropyloxymethylcarbamoyl group, a 2-methoxyethylcarbamoyl group, a 2-ethoxyethylcarbamoyl group, a cyanomethylcarbamoyl group, a 1-cyanoethylcarbamoyl group, a 2-cyanoethylcarbamoyl group, a cyclopropylmethylcarbamoyl group, a cyclobutylmethylcarbamoyl group, a benzylcarbamoyl group, a 1-phenethylcarbamoyl group, a 1-difluoroethylcarbamoyl group, a 2,2-difluoroethylcarbamoyl group, a 2,2,2-trifluoroethylcarbamoyl group, a 2,2,3,3,3-pentafluoropropylcarbamoyl group, a 2,2,3,3,4,4,4-heptafluorobutylcarbamoyl group, a 3,3,4,4,5,5,5-heptafluoropentylcarbamoyl group, a cyclopropylcarbamoyl group, a 1-cyanocyclopropylcarbamoyl group, an allylcarbamoyl group, a 3,3-difluoroallylcarbamoyl group, a propargylcarbamoyl group, a methoxycarbamoyl group, a trifluoroethoxycarbamoyl group, a phenylcarbamoyl group, a 4-cyanophenylcarbamoyl group, a 2-fluorophenylcarbamoyl group, a 3-fluorophenylcarbamoyl group, a 4-fluorophenylcarbamoyl group, a 2-chlorophenylcarbamoyl group, a 3-chlorophenylcarbamoyl group, a 4-chlorophenylcarbamoyl group, a 2-trifluoromethylphenylcarbamoyl group, a 3-trifluoromethylphenylcarbamoyl group, a 4-trifluoromethylphenylcarbamoyl group, a 2-methoxyphenylcarbamoyl group, a 3-methoxyphenylcarbamoyl group, a 4-methoxyphenylcarbamoyl group, a pyrrolidinecarbonyl group, a piperidinecarbonyl group, a piperazinecarbonyl group, a morpholinecarbonyl group, a thiomorpholinecarbonyl group, a dioxothiomorpholinecarbonyl group, a thiazolidinecarbonyl group, an azepanecarbonyl group, an azocancarbonyl group, a dimethylcarbamoyl group, an ethylmethylcarbamoyl group, a diethylcarbamoyl group, a methylpropylcarbamoyl group, an ethylpropylcarbamoyl group, an isopropylmethylcarbamoyl group, an isopropylethylcarbamoyl group, a diisopropylcarbamoyl group, a butylmethylcarbamoyl group, a butylethylcarbamoyl group, a sec-butylmethylcarbamoyl group, a sec-butylethylcarbamoyl group, an isobutylmethylcarbamoyl group, an isobutylethyl carbamoyl group, a t-butylmethylcarbamoyl group, a t-butylethylcarbamoyl group, a methylpentylcarbamoyl group, an ethylpentylcarbamoyl group, an isopentylmethylcarbamoyl group, an isopentylethylcarbamoyl group, a methyl(2-methylbutyl)carbamoyl group, an ethyl(2-methylbutyl)carbamoyl group, a hexylmethylcarbamoyl group, a hexylethylcarbamoyl group, a (methoxymethyl)methylcarbamoyl group, an (ethoxymethyl)methylcarbamoyl group, a (methoxyethyl)methylcarbamoyl group, an (ethoxyethyl)methylcarbamoyl group, a (propyloxymethyl)methylcarbamoyl group, an (isopropyloxymethyl)methylcarbamoyl group, a (methoxymethyl)ethylcarbamoyl group, an (ethoxymethyl)ethylcarbamoyl group, a (cyanomethyl)methylcarbamoyl group, a (cyanomethyl)ethylcarbamoyl group, a (cyclopropylmethyl)methylcarbamoyl group, a (cyclopropylmethyl)ethylcarbamoyl group, a (benzyl)methylcarbamoyl group, a methyl(pyridin-3-ylmethyl)carbamoyl group, a methyl(2,2,2-trifluoroethyl)carbamoyl group, a methyl(2,2,3,3,3-pentafluoropropyl)carbamoyl group, a methyl(2,2,3,3,4,4,4-heptafluorobutyl)carbamoyl group, a methyl(3,3,4,4,5,5,5-heptafluoropentyl)carbamoyl group, a (cyclopropyl)methylcarbamoyl group, a (cyclopropyl)ethylcarbamoyl group, a (1-cyanocyclopropyl)methylcarbamoyl group, a (1-cyanocyclopropyl)ethylcarbamoyl group, a (cyclobutyl)methylcarbamoyl group, a (cyclopentyl)methylcarbamoyl group, a (cyclohexyl)methylcarbamoyl group, an (allyl)methylcarbamoyl group, a methyl(2-propyn-1-yl)carbamoyl group, an ethyl(2-propyn-1-yl)carbamoyl group, a (methoxy) methylcarbamoyl group, an (ethoxy)methylcarbamoyl group, a (trifluoroethoxy)methylcarbamoyl group, a methylphenylcarbamoyl group, a diphenylcarbamoyl group, a methyl(pyridin-2-yl)carbamoyl group, a methyl(6-methyl-pyridin-2-yl)carbamoyl group, a methyl(pyridin-3-yl)carbamoyl group, or a methyl(pyridin-4-yl)carbamoyl group, more preferably a carbamoyl group, a methylcarbamoyl group, an ethylcarbamoyl group, a pyrrolidinecarbonyl group, a piperidinecarbonyl group, a 4-cyanopiperidinecarbonyl group, a 4-fluoropiperidinecarbonyl group, a 4,4-difluoropiperidinecarbonyl group, a 4-methylpiperidinecarbonyl group, a 4-trifluoromethylpiperidinecarbonyl group, a 4-methoxycarbonylpiperidinecarbonyl group, a piperazinecarbonyl group, a morpholinecarbonyl group, a thiomorpholinecarbonyl group, a dioxothiomorpholinecarbonyl group, a thiazolidinecarbonyl group, an azepanecarbonyl group, an azocancarbonyl group, a dimethylcarbamoyl group, an ethylmethylcarbamoyl group, a diethylcarbamoyl group, a methylpropylcarbamoyl group, an ethylpropylcarbamoyl group, an isopropylmethylcarbamoyl group, an isopropylethylcarbamoyl group, a diisopropylcarbamoyl group, a butylmethylcarbamoyl group, a butylethylcarbamoyl group, a sec-butylmethylcarbamoyl group, a sec-butylethylcarbamoyl group, an isobutylmethylcarbamoyl group, an isobutylethylcarbamoyl group, a t-butylmethylcarbamoyl group, a t-butylethylcarbamoyl group, a methylpentylcarbamoyl group, an ethylpentylcarbamoyl group, an isopentylmethylcarbamoyl group, an isopentylethylcarbamoyl group, a methyl(2-methylbutyl)carbamoyl group, an ethyl(2-methylbutyl)carbamoyl group, a hexylmethylcarbamoyl group, a hexylethylcarbamoyl group, a (methoxymethyl)methylcarbamoyl group, an (ethoxymethyl)methylcarbamoyl group, a (methoxyethyl)methylcarbamoyl group, an (ethoxyethyl)methylcarbamoyl group, a (propyloxymethyl)methylcarbamoyl group, an (isopropyloxymethyl)methylcarbamoyl group, a (methoxymethyl)ethylcarbamoyl group, an (ethoxymethyl)ethylcarbamoyl group, a (cyanomethyl)methylcarbamoyl group, a (cyanomethyl)ethylcarbamoyl group, a (cyclopropylmethyl)methylcarbamoyl group, a (cyclopropylmethyl)ethylcarbamoyl group, a (benzyl)methylcarbamoyl group, a methyl(pyridin-3-ylmethyl)carbamoyl group, a methyl(2,2,2-trifluoroethyl)carbamoyl group, a methyl(2,2,3,3,3-pentafluoropropyl)carbamoyl group, a methyl(2,2,3,3,4,4,4-heptafluorobutyl)carbamoyl group, a methyl(3,3,4,4,5,5,5-heptafluoropentyl)carbamoyl group, a (cyclopropyl)methylcarbamoyl group, a (cyclopropyl)ethylcarbamoyl group, a (1-cyanocyclopropyl)methylcarbamoyl group, a (1-cyanocyclopropyl)ethylcarbamoyl group, a (cyclobutyl)methylcarbamoyl group, a (cyclopentyl)methylcarbamoyl group, a (cyclohexyl)methylcarbamoyl group, an (allyl)methylcarbamoyl group, a methyl(2-propyn-1-yl)carbamoyl group, an ethyl(2-propyn-1-yl)carbamoyl group, a (methoxy)methylcarbamoyl group, an (ethoxy)methylcarbamoyl group, a (trifluoroethoxy)methylcarbamoyl group, a methylphenylcarbamoyl group, a diphenylcarbamoyl group, a methyl(pyridin-2-yl)carbamoyl group, a methyl(6-methyl-pyridin-2-yl)carbamoyl group, a methyl(pyridin-3-yl)carbamoyl group, or a methyl(pyridin-4-yl)carbamoyl group,
still more preferably a carbamoyl group, a methylcarbamoyl group, a pyrrolidinecarbonyl group, a piperidinecarbonyl group, a dimethylcarbamoyl group, or an ethylmethylcarbamoyl group, and
particularly preferably a methylcarbamoyl group or a dimethylcarbamoyl group.

Each term of “Rx3C(═O)—” (where Rx3 represents a hydrogen atom, a hydroxyl group, a halogen atom, a C1 to C6 alkyl group optionally substituted with a substituent A, a C1 to C6 haloalkyl group, a C3 to C8 cycloalkyl group optionally substituted with a substituent B, a C2 to C6 alkenyl group optionally substituted with a substituent A, a C2 to C6 haloalkenyl group, a C2 to C6 alkynyl group optionally substituted with a substituent A, a C2 to C6 haloalkynyl group, a phenyl group optionally substituted with 0 to 5 substituents B, or a 5- or 6-membered aromatic heterocyclic group optionally substituted with 0 to 4 substituents B) in R4 in Formula (1-1) or Formula (1-2) has the same meaning as above.

The “Rx3C(═O)—” is preferably a formyl group, an acetyl group, a propionyl group, an isopropionyl group, a butyryl group, an isobutyryl group, a t-butyryl group, a pentanoyl group, a 3-methyl-1-butyryl group, a 3-ethyl-1-pentanoyl group, a hexanoyl group, a (1H-pyrazol-1-yl)acetyl group, a (1H-1,2,4-triazol-l-yl)acetyl group, a cyclopropylmethylcarbonyl group, a 2-fluoroacetyl group, a 2-chloroacetyl group, a 2-bromoacetyl group, a 2,2-difluoroacetyl group, a 2,2-dichloroacetyl group, a 2,2-dibromoacetyl group, a 2,2,2-trifluoroacetyl group, a 2-chloro-2,2-difluoroacetyl group, a 2,2-dichloro-2-fluoroacetyl group, a 2,2,2-trichloroacetyl group, a 3,3-difluoropropionyl group, a 2,2,3,3-tetrafluoropropionyl group, a 2,2,3,3,3-pentafluoropropionyl group, a 2,2-difluoropropionyl group, a 3,3,3-trifluoropropionyl group, a 3-chloro-2,2,3,3-tetrafluoropropionyl group, a 3,3,3-trifluoro-2,2-dimethylpropionyl group, a 4,4,4-trifluorobutyryl group, a 2,2,3,3,4,4-hexafluorobutyryl group, a 2,2,3,3,4,4,4-heptafluorobutyryl group, a 4,4,4-trifluoro-3-(trifluoromethyl)butyryl group, a 2,2,3,3,4,4,5,5-octafluoropentanoyl group, a 2,2,3,3,4,4,5,5,5-nonafluoropentanoyl group, a 2,2,3,3,4,4,5,5,6,6-decafluorohexanoyl group, a 2,2,3,3,4,4,5,5,6,6,6-undecafluorohexanoyl group, a cyclopropylcarbonyl group, a 1-fluorocyclopropyl-1-carbonyl group, a 1-trifluoromethylcyclopropyl-1-carbonyl group, a 1-cyanocyclopropyl-1-carbonyl group, a 1-methylcyclopropyl-1-carbonyl group, a 2,2,3,3-tetramethyl-1-cyclopropylcarbonyl group, a cyclobutylcarbonyl group, a 1-trifluoromethylcyclobutyl-1-carbonyl group, a cyclopentylcarbonyl group, a cyclohexylcarbonyl group, an acryloyl group, a methacryloyl group, a 2-fluoroacryloyl group, a 2-chloroacryloyl group, a 3-ethoxyacryloyl group, a benzoyl group, a 2-fluorobenzoyl group, a 3-fluorobenzoyl group, a 4-fluorobenzoyl group, a 2-chlorobenzoyl group, a 3-chlorobenzoyl group, a 4-chlorobenzoyl group, a 2-bromobenzoyl group, a 3-bromobenzoyl group, a 4-bromobenzoyl group, a 2-methylbenzoyl group, a 3-methylbenzoyl group, a 4-methylbenzoyl group, a 2-ethylbenzoyl group, a 3-ethylbenzoyl group, a 4-ethylbenzoyl group, a 3,4-dimethylbenzoyl group, a 3,5-dimethylbenzoyl group, a 2-trifluoromethylbenzoyl group, a 3-trifluoromethylbenzoyl group, a 4-trifluoromethylbenzoyl group, a 2-methoxybenzoyl group, a 3-methoxybenzoyl group, a 4-methoxybenzoyl group, a 2-trifluoromethoxybenzoyl group, a 3-trifluoromethoxybenzoyl group, a 4-trifluoromethoxybenzoyl group, a picolinoyl group, a 3-chloropicolinoyl group, a 4-chloropicolinoyl group, a 5-chloropicolinoyl group, a 6-chloropicolinoyl group, a 3-methylpicolinoyl group, a 4-methylpicolinoyl group, a 5-methylpicolinoyl group, a 6-methylpicolinoyl group, a 3-trifluoromethylpicolinoyl group, a 4-trifluoromethylpicolinoyl group, a 5-trifluoromethylpicolinoyl group, a 6-trifluoromethylpicolinoyl group, a nicotinoyl group, a 2-chloronicotinoyl group, a 4-chloronicotinoyl group, a 5-chloronicotinoyl group, a 6-chloronicotinoyl group, a 2-methylnicotinoyl group, a 4-methylnicotinoyl group, a 5-methylnicotinoyl group, a 6-methylnicotinoyl group, a 2-trifluoromethylnicotinoyl group, a 4-trifluoromethylnicotinoyl group, a 5-trifluoromethylnicotinoyl group, a 6-trifluoromethylnicotinoyl group, an isonicotinoyl group, a 2-chloroisonicotinoyl group, a 3-chloroisonicotinoyl group, a pyrimidine-2-carbonyl group, a pyrimidine-4-carbonyl group, a 2-chloro-pyrimidine-4-carbonyl group, a 2-methyl-pyrimidine-4-carbonyl group, a 2-trifluoromethyl-pyrimidine-4-carbonyl group, a 6-methyl-pyrimidine-4-carbonyl group, a pyrimidine-5-carbonyl group, or a pyrazine-2-carbonyl group,
more preferably a formyl group, an acetyl group, a propionyl group, an isopropionyl group, a butyryl group, an isobutyryl group, a t-butyryl group, a pentanoyl group, a 3-methyl-1-butyryl group, a 3-ethyl-1-pentanoyl group, a hexanoyl group, a (1H-pyrazol-1-yl)acetyl group, a (1H-1,2,4-triazol-1-yl)acetyl group, a cyclopropylmethylcarbonyl group, a 2-fluoroacetyl group, a 2-chloroacetyl group, a 2-bromoacetyl group, a 2,2-difluoroacetyl group, a 2,2-dichloroacetyl group, a 2,2-dibromoacetyl group, a 2,2,2-trifluoroacetyl group, a 2-chloro-2,2-difluoroacetyl group, a 2,2-dichloro-2-fluoroacetyl group, a 2,2,2-trichloroacetyl group, a 3,3-difluoropropionyl group, a 2,2,3,3-tetrafluoropropionyl group, a 2,2,3,3,3-pentafluoropropionyl group, a 2,2-difluoropropionyl group, a 3,3,3-trifluoropropionyl group, a 3-chloro-2,2,3,3-tetrafluoropropionyl group, a 3,3,3-trifluoro-2,2-dimethylpropionyl group, a 4,4,4-trifluorobutyryl group, a 2,2,3,3,4,4-hexafluorobutyryl group, a 2,2,3,3,4,4,4-heptafluorobutyryl group, a 4,4,4-trifluoro-3-(trifluoromethyl)butyryl group, a 2,2,3,3,4,4,5,5-octafluoropentanoyl group, a 2,2,3,3,4,4,5,5,5-nonafluoropentanoyl group, a 2,2,3,3,4,4,5,5,6,6-decafluorohexanoyl group, a 2,2,3,3,4,4,5,5,6,6,6-undecafluorohexanoyl group, a cyclopropylcarbonyl group, a 1-fluorocyclopropyl-1-carbonyl group, a 1-trifluoromethylcyclopropyl-1-carbonyl group, a 1-cyanocyclopropyl-1-carbonyl group, a 1-methylcyclopropyl-1-carbonyl group, a 2,2,3,3-tetramethyl-1-cyclopropylcarbonyl group, a cyclobutylcarbonyl group, a 1-trifluoromethylcyclobutyl-1-carbonyl group, a cyclopentylcarbonyl group, a cyclohexylcarbonyl group, an acryloyl group, a 2-fluoroacryloyl group, a 3-ethoxyacryloyl group, a benzoyl group, a 2-fluorobenzoyl group, a 3-fluorobenzoyl group, a 4-fluorobenzoyl group, a 2-chlorobenzoyl group, a 3-chlorobenzoyl group, a 4-chlorobenzoyl group, a 2-bromobenzoyl group, a 3-bromobenzoyl group, a 4-bromobenzoyl group, a 2-methylbenzoyl group, a 3-methylbenzoyl group, a 4-methylbenzoyl group, a 4-ethylbenzoyl group, a 3,4-dimethylbenzoyl group, a 3,5-dimethylbenzoyl group, a 2-trifluoromethylbenzoyl group, a 3-trifluoromethylbenzoyl group, a 4-trifluoromethylbenzoyl group, a 2-methoxybenzoyl group, a 3-methoxybenzoyl group, a 4-methoxybenzoyl group, a 2-trifluoromethoxybenzoyl group, a 3-trifluoromethoxybenzoyl group, a 4-trifluoromethoxybenzoyl group, a picolinoyl group, a 3-chloropicolinoyl group, a 4-chloropicolinoyl group, a 5-chloropicolinoyl group, a 6-chloropicolinoyl group, a 3-methylpicolinoyl group, a 4-methylpicolinoyl group, a 5-methylpicolinoyl group, a 6-methylpicolinoyl group, a 3-trifluoromethylpicolinoyl group, a 4-trifluoromethylpicolinoyl group, a 5-trifluoromethylpicolinoyl group, a 6-trifluoromethylpicolinoyl group, a nicotinoyl group, a 2-chloronicotinoyl group, a 4-chloronicotinoyl group, a 5-chloronicotinoyl group, a 6-chloronicotinoyl group, a 2-methylnicotinoyl group, a 4-methylnicotinoyl group, a 5-methylnicotinoyl group, a 6-methylnicotinoyl group, a 2-trifluoromethylnicotinoyl group, a 4-trifluoromethylnicotinoyl group, a 5-trifluoromethylnicotinoyl group, a 6-trifluoromethylnicotinoyl group, an isonicotinoyl group, a 2-chloroisonicotinoyl group, a 3-chloroisonicotinoyl group, a pyrimidine-2-carbonyl group, a pyrimidine-4-carbonyl group, a 2-chloro-pyrimidine-4-carbonyl group, a 2-methyl-pyrimidine-4-carbonyl group, a 2-trifluoromethyl-pyrimidine-4-carbonyl group, a 6-methyl-pyrimidine-4-carbonyl group, a pyrimidine-5-carbonyl group, or a pyrazine-2-carbonyl group, still more preferably an acetyl group, a propionyl group, an isopropionyl group, a butyryl group, an isobutyryl group, a t-butyryl group, a pentanoyl group, a 3-methyl-1-butyryl group, a 3-ethyl-1-pentanoyl group, a hexanoyl group, a (1H-pyrazol-1-yl)acetyl group, a (1H-1,2,4-triazol-1-yl)acetyl group, a cyclopropylmethylcarbonyl group, a 2-fluoroacetyl group, a 2-chloroacetyl group, a 2-bromoacetyl group, a 2,2-difluoroacetyl group, a 2,2-dichloroacetyl group, a 2,2-dibromoacetyl group, a 2,2,2-trifluoroacetyl group, a 2-chloro-2,2-difluoroacetyl group, a 2,2-dichloro-2-fluoroacetyl group, a 2,2,2-trichloroacetyl group, a 3,3-difluoropropionyl group, a 2,2,3,3-tetrafluoropropionyl group, a 2,2,3,3,3-pentafluoropropionyl group, a 2,2-difluoropropionyl group, a 3,3,3-trifluoropropionyl group, a 3-chloro-2,2,3,3-tetrafluoropropionyl group, a 3,3,3-trifluoro-2,2-dimethylpropionyl group, a 4,4,4-trifluorobutyryl group, a 2,2,3,3,4,4-hexafluorobutyryl group, a 2,2,3,3,4,4,4-heptafluorobutyryl group, a 4,4,4-trifluoro-3-(trifluoromethyl)butyryl group, a 2,2,3,3,4,4,5,5-octafluoropentanoyl group, a 2,2,3,3,4,4,5,5,5-nonafluoropentanoyl group, a 2,2,3,3,4,4,5,5,6,6-decafluorohexanoyl group, a 2,2,3,3,4,4,5,5,6,6,6-undecafluorohexanoyl group, a cyclopropylcarbonyl group, a 1-fluorocyclopropyl-1-carbonyl group, a 1-trifluoromethylcyclopropyl-1-carbonyl group, a 1-cyanocyclopropyl-1-carbonyl group, a 1-methylcyclopropyl-1-carbonyl group, a 2,2,3,3-tetramethyl-1-cyclopropylcarbonyl group, a cyclobutylcarbonyl group, a 1-trifluoromethylcyclobutyl-1-carbonyl group, a cyclopentylcarbonyl group, a cyclohexylcarbonyl group, an acryloyl group, a 2-fluoroacryloyl group, a 3-ethoxyacryloyl group, a benzoyl group, a 2-fluorobenzoyl group, a 3-fluorobenzoyl group, a 4-fluorobenzoyl group, a 2-chlorobenzoyl group, a 3-chlorobenzoyl group, a 4-chlorobenzoyl group, a 2-methylbenzoyl group, a 3-methylbenzoyl group, a 4-methylbenzoyl group, a 4-ethylbenzoyl group, a 3,4-dimethylbenzoyl group, a 3,5-dimethylbenzoyl group, a 2-trifluoromethylbenzoyl group, a 3-trifluoromethylbenzoyl group, a 4-trifluoromethylbenzoyl group, a 2-methoxybenzoyl group, a 3-methoxybenzoyl group, a 4-methoxybenzoyl group, a 4-trifluoromethoxybenzoyl group, a picolinoyl group, a 3-chloropicolinoyl group, a 4-chloropicolinoyl group, a 5-chloropicolinoyl group, a 6-chloropicolinoyl group, a 6-methylpicolinoyl group, a 3-trifluoromethylpicolinoyl group, a 4-trifluoromethylpicolinoyl group, a 5-trifluoromethylpicolinoyl group, a 6-trifluoromethylpicolinoyl group, a nicotinoyl group, a 2-chloronicotinoyl group, a 4-chloronicotinoyl group, a 5-chloronicotinoyl group, a 6-chloronicotinoyl group, a 6-trifluoromethylnicotinoyl group, a 2-chloroisonicotinoyl group, a 3-chloroisonicotinoyl group, a pyrimidine-2-carbonyl group, a pyrimidine-4-carbonyl group, a 2-chloro-pyrimidine-4-carbonyl group, a 2-methyl-pyrimidine-4-carbonyl group, a 2-trifluoromethyl-pyrimidine-4-carbonyl group, a 6-methyl-pyrimidine-4-carbonyl group, or a pyrazine-2-carbonyl group, and particularly preferably an acetyl group, a propionyl group, an isopropionyl group, a butyryl group, an isobutyryl group, a pentanoyl group, a 3-methyl-1-butyryl group, a 3-ethyl-1-pentanoyl group, a (1H-pyrazol-1-yl)acetyl group, a (11H-1,2,4-triazol-1-yl)acetyl group, a cyclopropylmethylcarbonyl group, a 2-chloroacetyl group, a 2,2-difluoroacetyl group, a 2,2-dichloroacetyl group, a 2,2,2-trifluoroacetyl group, a 2-chloro-2,2-difluoroacetyl group, a 2,2-dichloro-2-fluoroacetyl group, a 2,2,2-trichloroacetyl group, a 3,3-difluoropropionyl group, a 2,2,3,3-tetrafluoropropionyl group, a 2,2,3,3,3-pentafluoropropionyl group, a 2,2-difluoropropionyl group, a 3,3,3-trifluoropropionyl group, a 3-chloro-2,2,3,3-tetrafluoropropionyl group, a 3,3,3-trifluoro-2,2-dimethylpropionyl group, a 4,4,4-trifluorobutyryl group, a 2,2,3,3,4,4,4-heptafluorobutyryl group, a 4,4,4-trifluoro-3-(trifluoromethyl)butyryl group, a 2,2,3,3,4,4,5,5,5-nonafluoropentanoyl group, a 2,2,3,3,4,4,5,5,6,6,6-undecafluorohexanoyl group, a cyclopropylcarbonyl group, a 1-fluorocyclopropyl-1-carbonyl group, a 1-trifluoromethycyclopropyl-1-carbonyl group, a 1-cyanocyclopropyl-1-carbonyl group, a 1-methylcyclopropyl-1-carbonyl group, a 2,2,3,3-tetramethyl-1-cyclopropylcarbonyl group, a cyclobutylcarbonyl group, a 1-trifluoromethylcyclobutyl-1-carbonyl group, a cyclopentylcarbonyl group, a cyclohexylcarbonyl group, an acryloyl group, a 2-fluoroacryloyl group, a 3-ethoxyacryloyl group, a benzoyl group, a 2-fluorobenzoyl group, a 3-fluorobenzoyl group, a 4-fluorobenzoyl group, a 2-chlorobenzoyl group, a 3-chlorobenzoyl group, a 4-chlorobenzoyl group, a 2-methylbenzoyl group, a 3-methylbenzoyl group, a 4-methylbenzoyl group, a 4-ethylbenzoyl group, a 3,4-dimethylbenzoyl group, a 3,5-dimethylbenzoyl group, a 2-trifluoromethylbenzoyl group, a 3-trifluoromethylbenzoyl group, a 4-trifluoromethylbenzoyl group, a 2-methoxybenzoyl group, a 3-methoxybenzoyl group, a 4-methoxybenzoyl group, a 4-trifluoromethoxybenzoyl group, a picolinoyl group, a 3-chloropicolinoyl group, a 4-chloropicolinoyl group, a 5-chloropicolinoyl group, a 6-chloropicolinoyl group, a 6-methylpicolinoyl group, a 6-trifluoromethylpicolinoyl group, a nicotinoyl group, a 2-chloronicotinoyl group, a 4-chloronicotinoyl group, a 5-chloronicotinoyl group, a 6-chloronicotinoyl group, a 2-chloroisonicotinoyl group, a 3-chloroisonicotinoyl group, a pyrimidine-2-carbonyl group, a pyrimidine-4-carbonyl group, a 2-chloro-pyrimidine-4-carbonyl group, a 2-methyl-pyrimidine-4-carbonyl group, a 2-trifluoromethyl-pyrimidine-4-carbonyl group, a 6-methyl-pyrimidine-4-carbonyl group, or a pyrazine-2-carbonyl group.

Each term of “Rx3OC(═O)—” (where Rx3 represents a hydrogen atom, a hydroxyl group, a halogen atom, a C1 to C6 alkyl group optionally substituted with a substituent A, a C1 to C6 haloalkyl group, a C3 to C8 cycloalkyl group optionally substituted with a substituent B, a C2 to C6 alkenyl group optionally substituted with a substituent A, a C2 to C6 haloalkenyl group, a C2 to C6 alkynyl group optionally substituted with a substituent A, a C2 to C6 haloalkynyl group, a phenyl group optionally substituted with 0 to 5 substituents B, or a 5- or 6-membered aromatic heterocyclic group optionally substituted with 0 to 4 substituents B) in R4 in Formula (1-1) or Formula (1-2) has the same meaning as above. The “Rx3OC(═O)—” is preferably a methoxycarbonyl group, an ethoxycarbonyl group, a propyloxycarbonyl group, an isopropyloxycarbonyl group, a t-butyloxycarbonyl group, a difluoromethoxycarbonyl group, a trifluoromethoxycarbonyl group, a 2,2,2-trifluoroethoxycarbonyl group, a cyclopropyloxycarbonyl group, a cyclopentyloxycarbonyl group, an allyloxycarbonyl group, a propargyloxycarbonyl group, a phenyloxycarbonyl group, or a 4-nitrophenyloxycarbonyl group, more preferably a methoxycarbonyl group, an ethoxycarbonyl group, a propyloxycarbonyl group, an isopropyloxycarbonyl group, a t-butyloxycarbonyl group, a cyclopropyloxycarbonyl group, a propargyloxycarbonyl group, a 2,2,2-trifluoroethoxycarbonyl group, a phenyloxycarbonyl group, or a 4-nitrophenyloxycarbonyl group, and particularly preferably a methoxycarbonyl group, an ethoxycarbonyl group, a propyloxycarbonyl group, a t-butyloxycarbonyl group, or a 2,2,2-trifluoroethoxycarbonyl group.

Each term of “Rx4S(O)p-” (where Rx4 represents a hydroxyl group, a halogen atom, a C1 to C6 alkyl group optionally substituted with a substituent A, a C1 to C6 haloalkyl group, a C3 to C8 cycloalkyl group optionally substituted with a substituent B, a C2 to C6 alkenyl group optionally substituted with a substituent A, a C2 to C6 haloalkenyl group, a C2 to C6 alkynyl group optionally substituted with a substituent A, a C2 to C6 haloalkynyl group, or a phenyl group optionally substituted with 0 to 5 substituents B, and p represents an integer of 0, 1, or 2) in R4 in Formula (1-1) or Formula (1-2) has the same meaning as above.

The “Rx4S(O)p-” is preferably a methylsulfonyl group, an ethylsulfonyl group, a trifluoromethylsulfonyl group, a 1,1,2,2,2-pentafluoroethylsulfonyl group, a cyclopropylsulfonyl group, a cyclopentylsulfonyl group, a phenylsulfonyl group, a 4-fluorophenylsulfonyl group, a 4-chlorophenylsulfonyl group, a 4-methylphenylsulfonyl group, or a 4-trifluoromethylphenylsulfonyl group, more preferably a methylsulfonyl group, an ethylsulfonyl group, a trifluoromethylsulfonyl group, a 4-methylphenylsulfonyl group, or a 4-trifluoromethylphenylsulfonyl group, and particularly preferably a methylsulfonyl group.

R5 in Formula (1-1) or Formula (1-2) represents a cyano group, a halogen atom, a C1 to C6 alkoxy group optionally substituted with a substituent A, a C1 to C6 haloalkoxy group, a C3 to C8 cycloalkoxy group optionally substituted with a substituent A, a C2 to C6 alkenyl group optionally substituted with a substituent A, a C2 to C6 haloalkenyl group, a C2 to C6 alkynyl group optionally substituted with a substituent A, a C2 to C6 haloalkynyl group, or Rx4S(O)p- (where Rx4 represents a hydroxyl group, a halogen atom, a C1 to C6 alkyl group optionally substituted with a substituent A, a C1 to C6 haloalkyl group, a C3 to C8 cycloalkyl group optionally substituted with a substituent B, a C2 to C6 alkenyl group optionally substituted with a substituent A, a C2 to C6 haloalkenyl group, a C2 to C6 alkynyl group optionally substituted with a substituent A, a C2 to C6 haloalkynyl group, or a phenyl group optionally substituted with 0 to 5 substituents B, and p represents an integer of 0, 1, or 2).

Among them, R5 is preferably a cyano group, a halogen atom, a C1 to C6 alkoxy group optionally substituted with a substituent A, a C1 to C6 haloalkoxy group, a C3 to C8 cycloalkoxy group optionally substituted with a substituent A, or Rx4S(O)p-(where Rx4 and p have the same meanings as above),

R5 is more preferably a cyano group, a halogen atom, a C1 to C6 alkoxy group optionally substituted with a substituent A, or Rx4S(O)p- (where Rx4 and p have the same meanings as above), and

R5 is particularly preferably Rx4S(O)p- (where Rx4 and p have the same meanings as above).

R5 in Formula (1-1) or Formula (1-2) contains a cyano group.

The “halogen atom” in R5 in Formula (1-1) or Formula (1-2) has the same meaning as above, and is preferably a fluorine atom, a chlorine atom, a bromine atom, or an iodine atom, more preferably a fluorine atom, a chlorine atom, or a bromine atom, and particularly preferably a chlorine atom or a bromine atom.

The C1 to C6 alkoxy group of the “C1 to C6 alkoxy group optionally substituted with a substituent A” in R5 in Formula (1-1) or Formula (1-2) has the same meaning as above, and is preferably a methoxy group, an ethoxy group, a propyloxy group, an isopropyloxy group, a butoxy group, or an isobutoxy group, more preferably a methoxy group, an ethoxy group, a propyloxy group, or an isopropyloxy group, and particularly preferably a methoxy group or an ethoxy group. When having the substituent described in the substituent A, a hydrogen atom in the C1 to C6 alkoxy group is optionally substituted with the substituent described in the substituent A.

The “C1 to C6 haloalkoxy group” in R5 in Formula (1-1) or Formula (1-2) has the same meaning as above, and is preferably a difluoromethoxy group, a trifluoromethoxy group, a 2,2-difluoroethoxy group, a 2,2,2-trifluoroethoxy group, a 1,2,2,2-tetrafluoroethoxy group, a 1,1,2,2,2-pentafluoroethoxy group, or a 1,1,2,2,3,3,3-heptafluoropropyloxy group, more preferably a trifluoromethoxy group, a 1,2,2,2-tetrafluoroethoxy group, a 1,1,2,2,2-pentafluoroethoxy group, or a 1,1,2,2,3,3,3-heptafluoropropyloxy group, and particularly preferably a trifluoromethoxy group or a 1,1,2,2,2-pentafluoroethoxy group.

The C3 to C8 cycloalkoxy group of the “C3 to C8 cycloalkoxy group optionally substituted with a substituent A” in R5 in Formula (1-1) or Formula (1-2) has the same meaning as above, and is preferably a cyclopropyloxy group, a cyclobutoxy group, a cyclopentyloxy group, or a cyclohexyloxy group, more preferably a cyclopropyloxy group or a cyclobutoxy group, and particularly preferably a cyclopropyloxy group. When having a substituent A, one hydrogen atom in the C3 to C8 cycloalkoxy group is substituted with the substituent A.

The C2 to C6 alkenyl group of the “C2 to C6 alkenyl group optionally substituted with a substituent A” in R5 in Formula (1-1) or Formula (1-2) has the same meaning as above, and is preferably a vinyl group, a 1-propenyl group, or an allyl group, more preferably a vinyl group or an allyl group, and particularly preferably an allyl group. When having a substituent A, one hydrogen atom in the C2 to C6 alkenyl group is substituted with the substituent A.

The “C2 to C6 haloalkenyl group” in R5 in Formula (1-1) or Formula (1-2) has the same meaning as above, and is preferably a 2-fluorovinyl group, a 2,2-difluorovinyl group, a 3-fluoroallyl group, or a 3,3-difluoroallyl group, more preferably a 3-fluoroallyl group or a 3,3-difluoroallyl group, and particularly preferably a 3,3-difluoroallyl group.

The C2 to C6 alkynyl group of the “C2 to C6 alkynyl group optionally substituted with a substituent A” in R5 in Formula (1-1) or Formula (1-2) has the same meaning as above, and is preferably an ethynyl group, a 1-propynyl group, a propargyl group, a 2-butynyl group, or a 3-butynyl group, more preferably an ethynyl group, a 1-propynyl group, or a propargyl group, and particularly preferably an ethynyl group or a propargyl group. When having a substituent A, one hydrogen atom in the C2 to C6 alkynyl group is substituted with the substituent A.

The “C2 to C6 haloalkynyl group” in R5 in Formula (1-1) or Formula (1-2) has the same meaning as above, and is preferably a 2-fluoroethynyl group, a 2-chloroethynyl group, a 2-bromoethynyl group, a 2-iodoethynyl group, a 3,3-difluoro-1-propynyl group, a 3-chloro-3,3-difluoro-1-propynyl group, a 3-bromo-3,3-difluoro-1-propynyl group, or a 3,3,3-trifluoro-1-propynyl group, more preferably a 2-fluoroethynyl group, a 2-chloroethynyl group, a 2-bromoethynyl group, a 2-iodoethynyl group, a 3,3-difluoro-1-propynyl group, or a 3,3,3-trifluoro-1-propynyl group, and particularly preferably a 2-fluoroethynyl group, a 2-chloroethynyl group, or a 3,3,3-trifluoro-1-propynyl group.

Each term of “Rx4S(O)p-” (where Rx4 represents a hydroxyl group, a halogen atom, a C1 to C6 alkyl group optionally substituted with a substituent A, a C1 to C6 haloalkyl group, a C3 to C8 cycloalkyl group optionally substituted with a substituent B, a C2 to C6 alkenyl group optionally substituted with a substituent A, a C2 to C6 haloalkenyl group, a C2 to C6 alkynyl group optionally substituted with a substituent A, a C2 to C6 haloalkynyl group, or a phenyl group optionally substituted with 0 to 5 substituents B, and p represents an integer of 0, 1, or 2) in R5 in Formula (1-1) or Formula (1-2) has the same meaning as above.

The “Rx4S(O)p-” is preferably a methylsulfanyl group, a methylsulfinyl group, a methylsulfonyl group, an ethylsulfanyl group, an ethylsulfinyl group, an ethylsulfonyl group, a propylsulfanyl group, a propylsulfinyl group, a propylsulfonyl group, a butylsulfanyl group, a butylsulfinyl group, a butylsulfonyl group, a pentylsulfanyl group, a pentylsulfinyl group, a pentylsulfonyl group, a hexylsulfanyl group, a hexylsulfinyl group, a hexylsulfonyl group, an isopropylsulfanyl group, an isopropylsulfinyl group, an isopropylsulfonyl group, a trifluoromethylsulfanyl group, a trifluoromethylsulfinyl group, a trifluoromethylsulfonyl group, a 1,1,2,2,2-pentafluoroethylsulfanyl group, a 1,1,2,2,2-pentafluoroethylsulfinyl group, a 1,1,2,2,2-pentafluoroethylsulfonyl group, a cyclopropylsulfanyl group, a cyclopropylsulfinyl group, a cyclopropylsulfonyl group, a cyclopentylsulfanyl group, a cyclopentylsulfinyl group, a cyclopentylsulfonyl group, a phenylsulfanyl group, a phenylsulfinyl group, a phenylsulfonyl group, a 4-fluorophenylsulfanyl group, a 4-fluorophenylsulfinyl group, a 4-fluorophenylsulfonyl group, a 4-chlorophenylsulfanyl group, a 4-chlorophenylsulfinyl group, a 4-chlorophenylsulfonyl group, a 4-methylphenylsulfanyl group, a 4-methylphenylsulfinyl group, a 4-methylphenylsulfonyl group, a 4-trifluoromethylphenylsulfanyl group, a 4-trifluoromethylphenylsulfinyl group, or a 4-trifluoromethylphenylsulfonyl group, more preferably a methylsulfanyl group, a methylsulfinyl group, a methylsulfonyl group, an ethylsulfanyl group, an ethylsulfinyl group, an ethylsulfonyl group, a propylsulfanyl group, a propylsulfinyl group, a propylsulfonyl group, a butylsulfanyl group, a butylsulfinyl group, a butylsulfonyl group, a pentylsulfanyl group, a pentylsulfinyl group, a pentylsulfonyl group, a hexylsulfanyl group, a hexylsulfinyl group, a hexylsulfonyl group, an isopropylsulfanyl group, an isopropylsulfinyl group, an isopropylsulfonyl group, a trifluoromethylsulfanyl group, a trifluoromethylsulfinyl group, a trifluoromethylsulfonyl group, a 1,1,2,2,2-pentafluoroethylsulfanyl group, a 1,1,2,2,2-pentafluoroethylsulfinyl group, a 1,1,2,2,2-pentafluoroethylsulfonyl group, a cyclopropylsulfanyl group, a cyclopropylsulfinyl group, a cyclopropylsulfonyl group, a cyclopentylsulfanyl group, a cyclopentylsulfinyl group, or a cyclopentylsulfonyl group,
still more preferably a methylsulfanyl group, a methylsulfinyl group, a methylsulfonyl group, an ethylsulfanyl group, an ethylsulfinyl group, an ethylsulfonyl group, a propylsulfanyl group, a propylsulfinyl group, a propylsulfonyl group, an isopropylsulfanyl group, an isopropylsulfinyl group, an isopropylsulfonyl group, a trifluoromethylsulfanyl group, a trifluoromethylsulfinyl group, a trifluoromethylsulfonyl group, a 1,1,2,2,2-pentafluoroethylsulfanyl group, a 1,1,2,2,2-pentafluoroethylsulfinyl group, a 1,1,2,2,2-pentafluoroethylsulfonyl group, a cyclopropylsulfanyl group, a cyclopropylsulfinyl group, or a cyclopropylsulfonyl group, and particularly preferably an ethylsulfanyl group, an ethylsulfinyl group, or an ethylsulfonyl group.

Het represents Formula (Het).

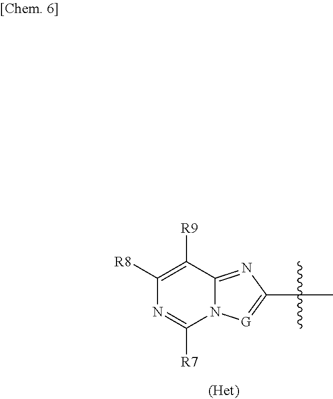

G in Formula (Het) represents C—R6 or a nitrogen atom. In addition, a binding site cut by a wavy line in Formula (Het) represents a site where Het is bonded in Formulas (1-1) and (1-2).

R6 in Formula (Het) represents a hydrogen atom, a cyano group, a halogen atom, a C1 to C6 alkyl group optionally substituted with a substituent A, a C1 to C6 haloalkyl group, a C3 to C8 cycloalkyl group optionally substituted with a substituent B, a C2 to C6 alkenyl group optionally substituted with a substituent A, a C2 to C6 haloalkenyl group, a C2 to C6 alkynyl group optionally substituted with a substituent A, or a C2 to C6 haloalkynyl group.

Among them, R6 is preferably a hydrogen atom, a cyano group, a halogen atom, a C1 to C6 alkyl group optionally substituted with a substituent A, a C1 to C6 haloalkyl group, or a C3 to C8 cycloalkyl group optionally substituted with a substituent B, R6 is more preferably a hydrogen atom, a halogen atom, a C1 to C6 alkyl group optionally substituted with a substituent A, or a C1 to C6 haloalkyl group, and R6 is particularly preferably a hydrogen atom, a halogen atom, or a C1 to C6 alkyl group optionally substituted with a substituent A.

R6 in Formula (Het) contains a hydrogen atom and a cyano group.

The “halogen atom” in R6 in Formula (Het) has the same meaning as above, and is preferably a fluorine atom, a chlorine atom, a bromine atom, or an iodine atom, more preferably a fluorine atom, a chlorine atom, or a bromine atom, and particularly preferably a chlorine atom or a bromine atom.

The C1 to C6 alkyl group of the “C1 to C6 alkyl group optionally substituted with a substituent A” in R6 in Formula (Het) has the same meaning as above, and is preferably a methyl group, an ethyl group, a propyl group, an isopropyl group, a butyl group, or an isobutyl group, more preferably a methyl group, an ethyl group, or a propyl group, and particularly preferably a methyl group. When having a substituent A, one hydrogen atom in the C1 to C6 alkyl group is substituted with the substituent A.

The “C1 to C6 haloalkyl group” in R6 in Formula (Het) has the same meaning as above, and is preferably a difluoromethyl group, a trifluoromethyl group, a dichloromethyl group, a dibromomethyl group, a 2,2-difluoroethyl group, a 2,2,2-trifluoroethyl group, a 3,3-difluoropropyl group, a 3,3,3- a trifluoropropyl group, or a 2,2,3,3,3-pentafluoropropyl group, more preferably a difluoromethyl group, a trifluoromethyl group, a 2,2-difluoroethyl group, a 2,2,2-trifluoroethyl group, a 3,3,3-trifluoropropyl group, or a 2,2,3,3,3-pentafluoropropyl group, and particularly preferably a difluoromethyl group, a trifluoromethyl group, a 2,2-difluoroethyl group, or a 2,2,2-trifluoroethyl group.

The C3 to C8 cycloalkyl group of the “C3 to C8 cycloalkyl group optionally substituted with a substituent B” in R6 in Formula (Het) has the same meaning as above, and is preferably a cyclopropyl group, a cyclobutyl group, a cyclopentyl group, or a cyclohexyl group, more preferably a cyclopropyl group or a cyclobutyl group, and particularly preferably a cyclopropyl group. When having a substituent A, one hydrogen atom in the C3 to C8 cycloalkyl group is substituted with the substituent A.

The C2 to C6 alkenyl group of the “C2 to C6 alkenyl group optionally substituted with a substituent A” in R6 in Formula (Het) has the same meaning as above, and is preferably a vinyl group, a 1-propenyl group, or an allyl group, more preferably a vinyl group or an allyl group, and particularly preferably an allyl group. When having a substituent A, one hydrogen atom in the C2 to C6 alkenyl group is substituted with the substituent A.

The “C2 to C6 haloalkenyl group” in R6 in Formula (Het) has the same meaning as above, is preferably a 2-fluorovinyl group, a 2,2-difluorovinyl group, a 3-fluoroallyl group, or a 3,3-difluoroallyl group, more preferably a 3-fluoroallyl group or a 3,3-difluoroallyl group, and particularly preferably a 3,3-difluoroallyl group.

The C2 to C6 alkynyl group of the “C2 to C6 alkynyl group optionally substituted with a substituent A” in R6 in Formula (Het) has the same meaning as above, and is preferably an ethynyl group, a 1-propynyl group, a propargyl group, a 2-butynyl group, or a 3-butynyl group, more preferably an ethynyl group, a 1-propynyl group, or a propargyl group, and particularly preferably an ethynyl group or a propargyl group. When having a substituent A, one hydrogen atom in the C2 to C6 alkynyl group is substituted with the substituent A.

The “C2 to C6 haloalkynyl group” in R6 in Formula (Het) has the same meaning as above, and is preferably a 2-fluoroethynyl group, a 2-chloroethynyl group, a 2-bromoethynyl group, a 2-iodoethynyl group, a 3,3-difluoro-1-propynyl group, a 3-chloro-3,3-difluoro-1-propynyl group, a 3-bromo-3,3-difluoro-1-propynyl group, or a 3,3,3-trifluoro-1-propynyl group, more preferably a 2-fluoroethynyl group, a 2-chloroethynyl group, a 2-bromoethynyl group, a 2-iodoethynyl group, a 3,3-difluoro-1-propynyl group, or a 3,3,3-trifluoro-1-propynyl group, and particularly preferably a 2-fluoroethynyl group, a 2-chloroethynyl group, or a 3,3,3-trifluoro-1-propynyl group.

R7 in Formula (Het) represents a hydrogen atom, a hydroxyl group, a cyano group, a halogen atom, a C1 to C6 alkyl group optionally substituted with a substituent A, a C1 to C6 haloalkyl group, a C3 to C8 cycloalkyl group optionally substituted with a substituent B, a C2 to C6 alkenyl group optionally substituted with a substituent A, a C2 to C6 haloalkenyl group, a C2 to C6 alkynyl group optionally substituted with a substituent A, a C2 to C6 haloalkynyl group, a C1 to C6 alkoxy group optionally substituted with a substituent A, a C1 to C6 haloalkoxy group, a C3 to C8 cycloalkoxy group optionally substituted with a substituent A, or Rx4S(O)p- (where Rx4 and p have the same meanings as above).

Among them, R7 is preferably a hydrogen atom, a hydroxyl group, a cyano group, a halogen atom, a C1 to C6 alkyl group optionally substituted with a substituent A, a C1 to C6 haloalkyl group, a C3 to C8 cycloalkyl group optionally substituted with a substituent B, a C1 to C6 alkoxy group optionally substituted with a substituent A, a C1 to C6 haloalkoxy group, a C3 to C8 cycloalkoxy group optionally substituted with a substituent A, or Rx4S(O)p- (where Rx4 and p have the same meanings as above), R7 is more preferably a hydrogen atom, a hydroxyl group, a cyano group, a halogen atom, a C1 to C6 alkyl group optionally substituted with a substituent A, a C1 to C6 haloalkyl group, a C3 to C8 cycloalkyl group optionally substituted with a substituent B, a C1 to C6 alkoxy group optionally substituted with a substituent A, a C1 to C6 haloalkoxy group, or a Rx4S(O)p- (where Rx4 and p have the same meanings as above), and R7 is particularly preferably a hydrogen atom, a hydroxyl group, a halogen atom, a C1 to C6 alkyl group optionally substituted with a substituent A, a C3 to C8 cycloalkyl group optionally substituted with a substituent B, a C1 to C6 alkoxy group optionally substituted with a substituent A, a C1 to C6 haloalkoxy group, or Rx4S(O)p-(where Rx4 and p have the same meanings as above).

R7 in Formula (Het) contains a hydrogen atom, a hydroxyl group, and a cyano group.

The halogen atom in R7 in Formula (Het) has the same meaning as above, and is preferably a fluorine atom, a chlorine atom, a bromine atom, or an iodine atom, more preferably a fluorine atom, a chlorine atom, or a bromine atom, and particularly preferably a chlorine atom.

The C1 to C6 alkyl group of the “C1 to C6 alkyl group optionally substituted with a substituent A” in R7 in Formula (Het) has the same meaning as above, and is preferably a methyl group, an ethyl group, a propyl group, an isopropyl group, a butyl group, or an isobutyl group, more preferably a methyl group, an ethyl group, or an isopropyl group, and particularly preferably a methyl group. When having a substituent A, one hydrogen atom in the C1 to C6 alkyl group is substituted with the substituent A.

The “C1 to C6 haloalkyl group” in R7 in Formula (Het) has the same meaning as above, and is preferably a difluoromethyl group, a trifluoromethyl group, a 2,2-difluoroethyl group, a 2,2,2-trifluoroethyl group, a 3,3-difluoropropyl group, or a 3,3,3-trifluoropropyl group, more preferably a difluoromethyl group, a trifluoromethyl group, a 2,2-difluoroethyl group, or a 2,2,2-trifluoroethyl group, and particularly preferably a difluoromethyl group or a trifluoromethyl group.

The C3 to C8 cycloalkyl group of the “C3 to C8 cycloalkyl group optionally substituted with a substituent B” in R7 in Formula (Het) has the same meaning as above, and is preferably a cyclopropyl group, a cyclobutyl group, a cyclopentyl group, or a cyclohexyl group, more preferably a cyclopropyl group or a cyclobutyl group, and particularly preferably a cyclopropyl group. When having a substituent B, one hydrogen atom in the C3 to C8 cycloalkyl group is substituted with the substituent B.

The C2 to C6 alkenyl group of the “C2 to C6 alkenyl group optionally substituted with a substituent A” in R7 in Formula (Het) has the same meaning as above, and is preferably a vinyl group, a 1-propenyl group, an allyl group, a 1-butenyl group, a 2-butenyl group, or a 3-butenyl group, more preferably a vinyl group, a 1-propenyl group, or an allyl group, and particularly preferably a vinyl group or an allyl group. When having a substituent A, one hydrogen atom in the C2 to C6 alkenyl group is substituted with the substituent A.

The “C2 to C6 haloalkenyl group” in R7 in Formula (Het) has the same meaning as above, is preferably a 2-fluorovinyl group, a 2,2-difluorovinyl group, a 2,2-dichlorovinyl group, a 3-fluoroallyl group, a 3,3-difluoroallyl group, or a 3,3-dichloroallyl group, more preferably a 2-fluorovinyl group, a 2,2-difluorovinyl group, a 3-fluoroallyl group, or a 3,3-difluoroallyl group, and particularly preferably a 2,2-difluorovinyl group or a 3,3-difluoroallyl group.

The C2 to C6 alkynyl group of the “C2 to C6 alkynyl group optionally substituted with a substituent A” in R7 in Formula (Het) has the same meaning as above, and is preferably an ethynyl group, a 1-propynyl group, a propargyl group, a 1-butynyl group, a 2-butynyl group, or a 3-butynyl group, more preferably an ethynyl group, a 1-propynyl group, or a propargyl group, and particularly preferably an ethynyl group. When having the substituent in the substituent A, one hydrogen atom in the C2 to C6 alkynyl group is substituted with the substituent A.

The “C2 to C6 haloalkynyl group” in R7 in Formula (Het) has the same meaning as above, and is preferably a 3,3-difluoro-1-propynyl group, a 3,3,3-trifluoro-1-propynyl group, a 4,4-difluoro-1-butynyl group, a 4,4-difluoro-2-butynyl group, a 4,4,4-trifluoro-1-butynyl group, or a 4,4,4-trifluoro-2-butynyl group, more preferably a 3,3-difluoro-1-propynyl group, a 3,3,3-trifluoro-1-propynyl group, or a 4,4,4-trifluoro-1-butynyl group, and particularly preferably a 3,3-difluoro-1-propynyl group or a 3,3,3-trifluoro-1-propynyl group.

The C1 to C6 alkoxy group of the “C1 to C6 alkoxy group optionally substituted with a substituent A” in R7 in Formula (Het) has the same meaning as above, and is preferably a methoxy group, an ethoxy group, a propyloxy group, an isopropyloxy group, a butoxy group, or an isobutoxy group, more preferably a methoxy group, an ethoxy group, a propyloxy group, or an isopropyloxy group, and particularly preferably a methoxy group, an ethoxy group, or a propyloxy group. When having the substituent described in the substituent A, a hydrogen atom in the C1 to C6 alkoxy group is optionally substituted with the substituent described in the substituent A.

The “C1 to C6 haloalkoxy group” in R7 in Formula (Het) has the same meaning as above, and is preferably a difluoromethoxy group, a trifluoromethoxy group, a 2,2-difluoroethoxy group, a 2,2,2-trifluoroethoxy group, a 1,2,2,2-tetrafluoroethoxy group, a 1,1,2,2,2-pentafluoroethoxy group, or a 1,1,2,2,3,3,3-heptafluoropropyloxy group, more preferably a trifluoromethoxy group, a 2,2,2-trifluoroethoxy group, a 1,2,2,2-tetrafluoroethoxy group, a 1,1,2,2,2-pentafluoroethoxy group, or a 1,1,2,2,3,3,3-heptafluoropropyloxy group, and particularly preferably a 2,2,2-trifluoroethoxy group.

The C3 to C8 cycloalkoxy group of the “C3 to C8 cycloalkoxy group optionally substituted with a substituent A” in R7 in Formula (Het) has the same meaning as above, and is preferably a cyclopropyloxy group, a cyclobutoxy group, a cyclopentyloxy group, or a cyclohexyloxy group, more preferably a cyclopropyloxy group or a cyclobutoxy group, and particularly preferably a cyclopropyloxy group. When having a substituent A, one hydrogen atom in the C3 to C8 cycloalkoxy group is substituted with the substituent A.

Each term of “Rx4S(O)p-” (where Rx4 represents a hydroxyl group, a halogen atom, a C1 to C6 alkyl group optionally substituted with a substituent A, a C1 to C6 haloalkyl group, a C3 to C8 cycloalkyl group optionally substituted with a substituent B, a C2 to C6 alkenyl group optionally substituted with a substituent A, a C2 to C6 haloalkenyl group, a C2 to C6 alkynyl group optionally substituted with a substituent A, a C2 to C6 haloalkynyl group, or a phenyl group optionally substituted with 0 to 5 substituents B, and p represents an integer of 0, 1, or 2) in R7 in Formula (Het) has the same meaning as above. Note that, regarding the “phenyl group optionally substituted with 0 to 5 substituents B”, when having the substituent B, hydrogen atoms in the phenyl group are optionally substituted with 1 to 5 independent substituents B. Rx4 is preferably a C1 to C6 alkyl group optionally substituted with a substituent A, a C1 to C6 haloalkyl group, a C3 to C8 cycloalkyl group optionally substituted with a substituent B, or a phenyl group optionally substituted with 0 to 5 substituents B, more preferably a C1 to C6 alkyl group optionally substituted with a substituent A, a C1 to C6 haloalkyl group, or a C3 to C8 cycloalkyl group optionally substituted with a substituent B, and particularly preferably a C1 to C6 haloalkyl group. The “Rx4S(O)p-” is preferably a methylsulfanyl group, a methylsulfinyl group, a methylsulfonyl group, an ethylsulfanyl group, an ethylsulfinyl group, an ethylsulfonyl group, a trifluoromethylsulfanyl group, a trifluoromethylsulfinyl group, a trifluoromethylsulfonyl group, a 1,1,2,2,2-pentafluoroethylsulfanyl group, a 1,1,2,2,2-pentafluoroethylsulfinyl group, a 1,1,2,2,2-pentafluoroethylsulfonyl group, a cyclopropylsulfanyl group, a cyclopropylsulfinyl group, a cyclopropylsulfonyl group, a phenylsulfanyl group, a phenylsulfinyl group, or a phenylsulfonyl group, more preferably a methylsulfanyl group, a methylsulfinyl group, a methylsulfonyl group, a trifluoromethylsulfanyl group, a trifluoromethylsulfinyl group, a trifluoromethylsulfonyl group, a 1,1,2,2,2-pentafluoroethylsulfanyl group, a 1,1,2,2,2-pentafluoroethylsulfinyl group, or a 1,1,2,2,2-pentafluoroethylsulfonyl group, still more preferably a methylsulfanyl group, a methylsulfinyl group, a methylsulfonyl group, a trifluoromethylsulfanyl group, a trifluoromethylsulfinyl group, or a trifluoromethylsulfonyl group, and particularly preferably a methylsulfanyl group or a methylsulfonyl group.

R8 in Formula (Het) represents a hydrogen atom, a hydroxyl group, a cyano group, a halogen atom, a C1 to C6 alkyl group optionally substituted with a substituent A, a C1 to C6 haloalkyl group, a C3 to C8 cycloalkyl group optionally substituted with a substituent B, a C1 to C6 alkoxy group optionally substituted with a substituent A, a C1 to C6 haloalkoxy group, a C3 to C8 cycloalkoxy group optionally substituted with a substituent A, or Rx4S(O)p- (where Rx4 and p have the same meanings as above).

Among them, R8 is preferably a hydrogen atom, a halogen atom, a C1 to C6 alkyl group optionally substituted with a substituent A, a C1 to C6 haloalkyl group, a C3 to C8 cycloalkyl group optionally substituted with a substituent B, a C1 to C6 haloalkoxy group, or Rx4S(O)p- (where Rx4 and p have the same meanings as above), R8 is more preferably a halogen atom, a C1 to C6 haloalkyl group, a C1 to C6 haloalkoxy group, or Rx4S(O)p- (where Rx4 and p have the same meanings as above), and R8 is particularly preferably a C1 to C6 haloalkyl group.

R8 in Formula (Het) contains a hydrogen atom, a hydroxyl group, and a cyano group.

The halogen atom in R8 in Formula (Het) has the same meaning as above, and is preferably a fluorine atom, a chlorine atom, a bromine atom, or an iodine atom, more preferably a fluorine atom, a chlorine atom, or a bromine atom, and particularly preferably a fluorine atom or a chlorine atom.

The C1 to C6 alkyl group of the “C1 to C6 alkyl group optionally substituted with a substituent A” in R8 in Formula (Het) has the same meaning as above, and is preferably a methyl group, an ethyl group, a propyl group, an isopropyl group, a butyl group, or an isobutyl group, more preferably a methyl group, an ethyl group, or an isopropyl group, and particularly preferably a methyl group or an ethyl group. When having a substituent A, one hydrogen atom in the C1 to C6 alkyl group is substituted with the substituent A.

The “C1 to C6 haloalkyl group” in R8 in Formula (Het) has the same meaning as above, and is preferably a difluoromethyl group, a trifluoromethyl group, a dichloromethyl group, a trichloromethyl group, a monobromomethyl group, a dibromomethyl group, a monoiodomethyl group, a diiodomethyl group, a chlorodifluoromethyl group, a bromodifluoromethyl group, a dichlorofluoromethyl group, a 1-fluoroethyl group, a 2-fluoroethyl group, a 1,1-difluoroethyl group, a 2,2-difluoroethyl group, a 2,2,2-trifluoroethyl group, a 1,1,2,2-tetrafluoroethyl group, a 1,2,2,2-tetrafluoroethyl group, a pentafluoroethyl group, a 2,2,2-trichloroethyl group, a 2-chloro-1,1,2,2-tetrafluoroethyl group, a 2-fluoropropyl group, a 3,3-difluoropropyl group, a 3,3,3-trifluoropropyl group, a 3,3,3-trichloropropyl group, a 2,3,3,3-tetrafluoropropyl group, a 2,2,3,3,3-pentafluoropropyl group, a 1,2,2,3,3,3-hexafluoropropyl group, a heptafluoropropyl group, a 1,1,1,3,3,3-hexafluoro-2-methylpropyl group, a 1,1,1-trifluoro-2-methylpropyl group, a 2-fluoroisopropyl group, a heptafluoroisopropyl group, a 2,2,2-trifluoro-1-(trifluoromethyl)ethyl group, a 2,2,3,3,4,4,4-heptafluorobutyl group, a 1,2,2,3,3,4,4,4-octafluorobutyl group, a nonafluorobutyl group, a nonafluoro-sec-butyl group, a 3,3,4,4,5,5,5-heptafluoropentyl group, a 2,3,3,4,4,5,5,5-octafluoropentyl group, a 2,2,3,3,4,4,5,5,5-nonafluoropentyl group, an undecafluoropentyl group, or a tridecafluorohexyl group,
more preferably a difluoromethyl group, a trifluoromethyl group, a dichloromethyl group, a trichloromethyl group, a monobromomethyl group, a dibromomethyl group, a monoiodomethyl group, a diiodomethyl group, a chlorodifluoromethyl group, a bromodifluoromethyl group, a dichlorofluoromethyl group, a 1-fluoroethyl group, a 2-fluoroethyl group, a 1,1-difluoroethyl group, a 2,2-difluoroethyl group, a 2,2,2-trifluoroethyl group, a 1,1,2,2-tetrafluoroethyl group, a 1,2,2,2-tetrafluoroethyl group, a pentafluoroethyl group, a 2,2,2-trichloroethyl group, a 2-chloro-1,1,2,2-tetrafluoroethyl group, a 2-fluoropropyl group, a 3,3-difluoropropyl group, a 3,3,3-trifluoropropyl group, a 3,3,3-trichloropropyl group, a 2,3,3,3-tetrafluoropropyl group, a 2,2,3,3,3-pentafluoropropyl group, a 1,2,2,3,3,3-hexafluoropropyl group, a heptafluoropropyl group, a 1,1,1,3,3,3-hexafluoro-2-methylpropyl group, a 1,1,1-trifluoro-2-methylpropyl group, a 2-fluoroisopropyl group, or a heptafluoroisopropyl group,
still more preferably a difluoromethyl group, a trifluoromethyl group, a dichloromethyl group, a trichloromethyl group, a monobromomethyl group, a dibromomethyl group, a monoiodomethyl group, a diiodomethyl group, a chlorodifluoromethyl group, a bromodifluoromethyl group, a dichlorofluoromethyl group, a 1-fluoroethyl group, a 2-fluoroethyl group, a 1,1-difluoroethyl group, a 2,2-difluoroethyl group, a 2,2,2-trifluoroethyl group, a 1,1,2,2-tetrafluoroethyl group, a 1,2,2,2-tetrafluoroethyl group, a pentafluoroethyl group, a 2,2,2-trichloroethyl group, a 2-chloro-1,1,2,2-tetrafluoroethyl group, a 2-fluoroisopropyl group, or a heptafluoropropyl group, and
particularly preferably a difluoromethyl group, a trifluoromethyl group, a dichloromethyl group, a chlorodifluoromethyl group, a pentafluoroethyl group, a 2-fluoroisopropyl group, or a heptafluoropropyl group.

The C3 to C8 cycloalkyl group of the “C3 to C8 cycloalkyl group optionally substituted with a substituent B” in R8 in Formula (Het) has the same meaning as above, and is preferably a cyclopropyl group, a cyclobutyl group, a cyclopentyl group, or a cyclohexyl group, more preferably a cyclopropyl group or a cyclobutyl group, and particularly preferably a cyclopropyl group. When having a substituent B, one hydrogen atom in the C3 to C8 cycloalkyl group is substituted with the substituent B.

The C1 to C6 alkoxy group of the “C1 to C6 alkoxy group optionally substituted with a substituent A” in R8 in Formula (Het) has the same meaning as above, and is preferably a methoxy group, an ethoxy group, a propyloxy group, an isopropyloxy group, a butoxy group, or an isobutoxy group, more preferably a methoxy group, an ethoxy group, a propyloxy group, or an isopropyloxy group, and particularly preferably a methoxy group or an ethoxy group. When having the substituent described in the substituent A, a hydrogen atom in the C1 to C6 alkoxy group is optionally substituted with the substituent described in the substituent A.

The “C1 to C6 haloalkoxy group” in R8 in Formula (Het) has the same meaning as above, and is preferably a difluoromethoxy group, a trifluoromethoxy group, a 2,2-difluoroethoxy group, a 2,2,2-trifluoroethoxy group, a 1,2,2,2-tetrafluoroethoxy group, a 1,1,2,2,2-pentafluoroethoxy group, or a 1,1,2,2,3,3,3-heptafluoropropyloxy group, more preferably a trifluoromethoxy group, a 1,2,2,2-tetrafluoroethoxy group, a 1,1,2,2,2-pentafluoroethoxy group, or a 1,1,2,2,3,3,3-heptafluoropropyloxy group, and particularly preferably a trifluoromethoxy group or a 1,1,2,2,2-pentafluoroethoxy group.

The C3 to C8 cycloalkoxy group of the “C3 to C8 cycloalkoxy group optionally substituted with a substituent A” in R8 in Formula (Het) has the same meaning as above, and is preferably a cyclopropyloxy group, a cyclobutoxy group, a cyclopentyloxy group, or a cyclohexyloxy group, more preferably a cyclopropyloxy group or a cyclobutoxy group, and particularly preferably a cyclopropyloxy group. When having a substituent A, one hydrogen atom in the C3 to C8 cycloalkoxy group is substituted with the substituent A.

Each term of “Rx4S(O)p-” (where Rx4 represents a hydroxyl group, a halogen atom, a C1 to C6 alkyl group optionally substituted with a substituent A, a C1 to C6 haloalkyl group, a C3 to C8 cycloalkyl group optionally substituted with a substituent B, a C2 to C6 alkenyl group optionally substituted with a substituent A, a C2 to C6 haloalkenyl group, a C2 to C6 alkynyl group optionally substituted with a substituent A, a C2 to C6 haloalkynyl group, or a phenyl group optionally substituted with 0 to 5 substituents B, and p represents an integer of 0, 1, or 2) in R8 in Formula (Het) has the same meaning as above. Note that, regarding the “phenyl group optionally substituted with 0 to 5 substituents B”, when having the substituent B, hydrogen atoms in the phenyl group are optionally substituted with 1 to 5 independent substituents B. Rx4 is preferably a C1 to C6 alkyl group optionally substituted with a substituent A, a C1 to C6 haloalkyl group, a C3 to C8 cycloalkyl group optionally substituted with a substituent B, or a phenyl group optionally substituted with 0 to 5 substituents B, more preferably a C1 to C6 alkyl group optionally substituted with a substituent A, a C1 to C6 haloalkyl group, or a C3 to C8 cycloalkyl group optionally substituted with a substituent B, and particularly preferably a C1 to C6 haloalkyl group. The “Rx4S(O)p-” is preferably a methylsulfanyl group, a methylsulfinyl group, a methylsulfonyl group, an ethylsulfanyl group, an ethylsulfinyl group, an ethylsulfonyl group, a trifluoromethylsulfanyl group, a trifluoromethylsulfinyl group, a trifluoromethylsulfonyl group, a 1,1,2,2,2-pentafluoroethylsulfanyl group, a 1,1,2,2,2-pentafluoroethylsulfinyl group, a 1,1,2,2,2-pentafluoroethylsulfonyl group, a cyclopropylsulfanyl group, a cyclopropylsulfinyl group, a cyclopropylsulfonyl group, a phenylsulfanyl group, a phenylsulfinyl group, or a phenylsulfonyl group, more preferably a methylsulfanyl group, a methylsulfinyl group, a methylsulfonyl group, a trifluoromethylsulfanyl group, a trifluoromethylsulfinyl group, a trifluoromethylsulfonyl group, a 1,1,2,2,2-pentafluoroethylsulfanyl group, a 1,1,2,2,2-pentafluoroethylsulfinyl group, or a 1,1,2,2,2-pentafluoroethylsulfonyl group, and particularly preferably a trifluoromethylsulfanyl group, a trifluoromethylsulfinyl group, or a trifluoromethylsulfonyl group.

R9 in Formula (Het) represents a hydrogen atom, a hydroxyl group, a cyano group, a halogen atom, a C1 to C6 alkyl group optionally substituted with a substituent A, a C1 to C6 haloalkyl group, a C3 to C8 cycloalkyl group optionally substituted with a substituent B, a C1 to C6 alkoxy group optionally substituted with a substituent A, a C1 to C6 haloalkoxy group, a C3 to C8 cycloalkoxy group optionally substituted with a substituent A, or Rx4S(O)p- (where Rx4 and p have the same meanings as above).

Among them, R9 is preferably a hydrogen atom, a halogen atom, a C1 to C6 alkyl group optionally substituted with a substituent A, a C1 to C6 haloalkyl group, or Rx4S(O)p- (where Rx4 and p have the same meanings as above),
R9 is more preferably a hydrogen atom, a halogen atom, or a C1 to C6 haloalkyl group, and
R9 is particularly preferably a hydrogen atom.

R9 in Formula (Het) contains a hydrogen atom, a hydroxyl group, and a cyano group.

The halogen atom in R9 in Formula (Het) has the same meaning as above, and is preferably a fluorine atom, a chlorine atom, a bromine atom, or an iodine atom, more preferably a fluorine atom, a chlorine atom, or a bromine atom, and particularly preferably a fluorine atom or a chlorine atom.

The C1 to C6 alkyl group of the “C1 to C6 alkyl group optionally substituted with a substituent A” in R9 in Formula (Het) has the same meaning as above, and is preferably a methyl group, an ethyl group, a propyl group, an isopropyl group, a butyl group, or an isobutyl group, more preferably a methyl group, an ethyl group, or an isopropyl group, and particularly preferably a methyl group or an ethyl group. When having a substituent A, one hydrogen atom in the C1 to C6 alkyl group is substituted with the substituent A.

The “C1 to C6 haloalkyl group” in R9 in Formula (Het) has the same meaning as above, and is preferably a difluoromethyl group, a trifluoromethyl group, a 2,2-difluoroethyl group, a 2,2,2-trifluoroethyl group, a 1,2,2,2-tetrafluoroethyl group, a 1,1,2,2,2-pentafluoroethyl group, or a 1,1,2,2,3,3,3-heptafluoropropyl group, more preferably a trifluoromethyl group, a 1,2,2,2-tetrafluoroethyl group, a 1,1,2,2,2-pentafluoroethyl group, or a 1,1,2,2,3,3,3-heptafluoropropyl group, and particularly preferably a trifluoromethyl group or a 1,1,2,2,2-pentafluoroethyl group.

The C3 to C8 cycloalkyl group of the “C3 to C8 cycloalkyl group optionally substituted with a substituent B” in R9 in Formula (Het) has the same meaning as above, and is preferably a cyclopropyl group, a cyclobutyl group, a cyclopentyl group, or a cyclohexyl group, more preferably a cyclopropyl group or a cyclobutyl group, and particularly preferably a cyclopropyl group. When having a substituent B, one hydrogen atom in the C3 to C8 cycloalkyl group is substituted with the substituent B.

The C1 to C6 alkoxy group of the “C1 to C6 alkoxy group optionally substituted with a substituent A” in R9 in Formula (Het) has the same meaning as above, and is preferably a methoxy group, an ethoxy group, a propyloxy group, an isopropyloxy group, a butoxy group, or an isobutoxy group, more preferably a methoxy group, an ethoxy group, a propyloxy group, or an isopropyloxy group, and particularly preferably a methoxy group or an ethoxy group. When having a substituent A, a hydrogen atom in the C1 to C6 alkoxy group is substituted with the substituent A.

The “C1 to C6 haloalkoxy group” in R9 in Formula (Het) has the same meaning as above, and is preferably a difluoromethoxy group, a trifluoromethoxy group, a 2,2-difluoroethoxy group, a 2,2,2-trifluoroethoxy group, a 1,2,2,2-tetrafluoroethoxy group, a 1,1,2,2,2-pentafluoroethoxy group, or a 1,1,2,2,3,3,3-heptafluoropropyloxy group, more preferably a trifluoromethoxy group, a 1,2,2,2-tetrafluoroethoxy group, a 1,1,2,2,2-pentafluoroethoxy group, or a 1,1,2,2,3,3,3-heptafluoropropyloxy group, and particularly preferably a trifluoromethoxy group or a 1,1,2,2,2-pentafluoroethoxy group.

The C3 to C8 cycloalkoxy group of the “C3 to C8 cycloalkoxy group optionally substituted with a substituent A” in R9 in Formula (Het) has the same meaning as above, and is preferably a cyclopropyloxy group, a cyclobutoxy group, a cyclopentyloxy group, or a cyclohexyloxy group, more preferably a cyclopropyloxy group or a cyclobutoxy group, and particularly preferably a cyclopropyloxy group. When having a substituent A, one hydrogen atom in the C3 to C8 cycloalkoxy group is substituted with the substituent A.

Each term of “Rx4S(O)p-” (where Rx4 represents a hydroxyl group, a halogen atom, a C1 to C6 alkyl group optionally substituted with a substituent A, a C1 to C6 haloalkyl group, a C3 to C8 cycloalkyl group optionally substituted with a substituent B, a C2 to C6 alkenyl group optionally substituted with a substituent A, a C2 to C6 haloalkenyl group, a C2 to C6 alkynyl group optionally substituted with a substituent A, a C2 to C6 haloalkynyl group, or a phenyl group optionally substituted with 0 to 5 substituents B, and p represents an integer of 0, 1, or 2) in R9 in Formula (Het) has the same meaning as above. Note that, regarding the “phenyl group optionally substituted with 0 to 5 substituents B”, when having the substituent B, hydrogen atoms in the phenyl group are optionally substituted with 1 to 5 independent substituents B. Rx4 is preferably a C1 to C6 alkyl group optionally substituted with a substituent A, a C1 to C6 haloalkyl group, a C3 to C8 cycloalkyl group optionally substituted with a substituent B, or a phenyl group optionally substituted with 0 to 5 substituents B, more preferably a C1 to C6 alkyl group optionally substituted with a substituent A, a C1 to C6 haloalkyl group, or a C3 to C8 cycloalkyl group optionally substituted with a substituent B, and particularly preferably a C1 to C6 haloalkyl group. The “Rx4S(O)p-” is preferably a methylsulfanyl group, a methylsulfinyl group, a methylsulfonyl group, an ethylsulfanyl group, an ethylsulfinyl group, an ethylsulfonyl group, a trifluoromethylsulfanyl group, a trifluoromethylsulfinyl group, a trifluoromethylsulfonyl group, a 1,1,2,2,2-pentafluoroethylsulfanyl group, a 1,1,2,2,2-pentafluoroethylsulfinyl group, a 1,1,2,2,2-pentafluoroethylsulfonyl group, a cyclopropylsulfanyl group, a cyclopropylsulfinyl group, a cyclopropylsulfonyl group, a phenylsulfanyl group, a phenylsulfinyl group, or a phenylsulfonyl group, more preferably a methylsulfanyl group, a methylsulfinyl group, a methylsulfonyl group, a trifluoromethylsulfanyl group, a trifluoromethylsulfinyl group, a trifluoromethylsulfonyl group, a 1,1,2,2,2-pentafluoroethylsulfanyl group, a 1,1,2,2,2-pentafluoroethylsulfinyl group, or a 1,1,2,2,2-pentafluoroethylsulfonyl group, and particularly preferably a trifluoromethylsulfanyl group, a trifluoromethylsulfinyl group, or a trifluoromethylsulfonyl group.

The “substituent A” is selected from the group consisting of a hydroxyl group, a cyano group, a nitro group, a halogen atom, a C3 to C8 cycloalkyl group, a C1 to C6 alkoxy group, a C1 to C6 haloalkoxy group, a C3 to C8 cycloalkoxy group, a phenyl group optionally substituted with 0 to 5 substituents C, a 5- or 6-membered aromatic heterocyclic group optionally substituted with 0 to 4 substituents C, and a 3- to 6-membered ring group containing one or two oxygen atoms.

Among them, the “substituent A” is selected from the group consisting of a cyano group, a halogen atom, a C3 to C8 cycloalkyl group, a C1 to C6 alkoxy group, a C1 to C6 haloalkoxy group, a phenyl group optionally substituted with 0 to 5 substituents C, a 5- or 6-membered aromatic heterocyclic group optionally substituted with 0 to 4 substituents C, and a 3- to 6-membered ring group containing one or two oxygen atoms.

Particularly, the “substituent A” is selected from the group consisting of a cyano group, a halogen atom, a C3 to C8 cycloalkyl group, a C1 to C6 alkoxy group, and a 5- or 6-membered aromatic heterocyclic group optionally substituted with 0 to 4 substituents C.

Preferred specific examples of the “substituent A” include a hydroxyl group; a cyano group; a nitro group;
as a halogen atom, a fluorine atom, a chlorine atom, a bromine atom, and an iodine atom;
as a C3 to C8 cycloalkyl group, a cyclopropyl group, a cyclobutyl group, a cyclopentyl group, and a cyclohexyl group;
as a C1 to C6 alkoxy group, a methoxy group, an ethoxy group, a propyloxy group, and an isopropyloxy group;
as a C1 to C6 haloalkoxy group, a difluoromethoxy group, a trifluoromethoxy group, a 2,2-difluoroethoxy group, a 2,2,2-trifluoroethoxy group, a 2,2,3,3-tetrafluoropropyloxy group, and a 2,2,3,3,3-pentafluoropropyloxy group;
a C3 to C8 cycloalkoxy group, a cyclopropyloxy group, a cyclobutyloxy group, a cyclopentyloxy group, and a cyclohexyloxy group;
as a phenyl group optionally substituted with 0 to 5 substituents C, a phenyl group, a cyanophenyl group, a fluorophenyl group, a chlorophenyl group, a dichlorophenyl group, a bromophenyl group, an iodophenyl group, a tolyl group, an ethylphenyl group, an isopropylphenyl group, a difluoromethylphenyl group, a trifluoromethylphenyl group, a pentafluoroethylphenyl group, a cyclopropylphenyl group, a cyclobutylphenyl group, a cyclopentylphenyl group, a methoxyphenyl group, an ethoxyphenyl group, an isopropyloxyphenyl group, a trifluoromethoxyphenyl group, a 2,2-difluoroethoxyphenyl group, a 2,2,2-trifluoroethoxyphenyl group, a 2,2,3,3,3-pentafluoropropyloxyphenyl group, a methylsulfanylphenyl group, a methylsulfinylphenyl group, a methylsulfonylphenyl group, an ethylsulfanylphenyl group, an ethylsulfinylphenyl group, an ethylsulfonylphenyl group, a trifluoromethylsulfanylphenyl group, a trifluoromethylsulfinylphenyl group, and a trifluoromethylsulfonylphenyl group;
as a 5- or 6-membered aromatic heterocyclic group optionally substituted with 0 to 4 substituents C, a pyridyl group, a pyridine-N-oxide group, a 2-fluoropyridyl group, a 3-fluoropyridyl group, a 4-fluoropyridyl group, a 2-chloropyridyl group, a 3-chloropyridyl group, a 4-chloropyridyl group, a 2-bromopyridyl group, a 3-bromopyridyl group, a 4-bromopyridyl group, a 2-iodopyridyl group, a 3-iodopyridyl group, a 4-iodopyridyl group, a 2-methylpyridyl group, a 3-methylpyridyl group, a 4-methylpyridyl group, a 2-trifluoromethylpyridyl group, a 3-trifluoromethylpyridyl group, a 4-trifluoromethylpyridyl group, a 2-cyclopropylpyridyl group, a 3-cyclopropylpyridyl group, a 4-cyclopropylpyridyl group, a 2-methoxypyridyl group, a 3-methoxypyridyl group, a 4-methoxypyridyl group, a pyridazinyl group, a 3-fluoropyridazinyl group, a 4-fluoropyridazinyl group, a 3-chloropyridazinyl group, a 4-chioropyridazinyl group, a 3-bromopyridazinyl group, a 4-bromopyridazinyl group, a 3-methylpyridazinyl group, a 4-methylpyridazinyl group, a pyrimidinyl group, a 2-fluoropyrimidinyl group, a 4-fluoropyrimidinyl group, a 5-fluoropyrimidinyl group, a 2-chloropyrimidinyl group, a 4-chloropyrimidinyl group, a 5-chloropyrimidinyl group, a 2-bromopyrimidinyl group, a 4-bromopyrimidinyl group, a 5-bromopyrimidinyl group, a 2-methylpyrimidinyl group, a 4-methylpyrimidinyl group, a 5-methylpyrimidinyl group, a 2-trifluoromethylpyrimidinyl group, a 4-trifluoromethylpyrimidinyl group, a 5-trifluoromethylpyrimidinyl group, a 2-methoxypyrimidinyl group, a 4-methoxypyrimidinyl group, a 5-methoxypyrimidinyl group, a pyrazinyl group, a 2-fluoropyrazinyl group, a 2-chloropyrazinyl group, a 2-bromopyrazinyl group, a 2-methylpyrazinyl group, a 2-trifluoromethylpyrazinyl group, a 2-methoxypyrazinyl group, a pyrazolyl group, a triazolyl group, a 1,2,4-oxadiazolyl group, a (5-trifluoromethyl)-1,2,4-oxadiazolyl group, and a thiazolyl group; and as a 3- to 6-membered ring group containing one or two oxygen atoms, an oxiran-2-yl group, an oxetan-3-yl group, a tetrahydrofuran-2-yl group, a tetrahydrofuran-3-yl group, a tetrahydro-2H-pyran-2-yl group, a tetrahydro-2H-pyran-3-yl group, a tetrahydro-2H-pyran-4-yl group, a 1,4-dioxan-2-yl group, a 3-dihydrofuran-2(3H)-one group, a 4-dihydrofuran-2(3H)-one group, a 3-tetrahydro-2H-pyran-2-one group, a 4-tetrahydro-2H-pyran-2-one group, and a 2-oxotetrahydrofuran-3-yl group.

More preferred specific examples of the “substituent A” include a cyano group;
as a halogen atom, a fluorine atom, a chlorine atom, and a bromine atom;
as a C3 to C8 cycloalkyl group, a cyclopropyl group, a cyclobutyl group, a cyclopentyl group, and a cyclohexyl group;
as a C1 to C6 alkoxy group, a methoxy group, an ethoxy group, a propyloxy group, and an isopropyloxy group;
as a C1 to C6 haloalkoxy group, a difluoromethoxy group, a trifluoromethoxy group, a 2,2-difluoroethoxy group, a 2,2,2-trifluoroethoxy group, a 2,2,3,3-tetrafluoropropyloxy group, and a 2,2,3,3,3-pentafluoropropyloxy group;
as a phenyl group optionally substituted with 0 to 5 substituents C, a phenyl group, a cyanophenyl group, a fluorophenyl group, a chlorophenyl group, a dichlorophenyl group, a bromophenyl group, an iodophenyl group, a tolyl group, an ethylphenyl group, an isopropylphenyl group, a difluoromethylphenyl group, a trifluoromethylphenyl group, a cyclopropylphenyl group, a methoxyphenyl group, an ethoxyphenyl group, an isopropyloxyphenyl group, a trifluoromethoxyphenyl group, a 2,2-difluoroethoxyphenyl group, a 2,2,2-trifluoroethoxyphenyl group, a 2,2,3,3,3-pentafluoropropyloxyphenyl group, a methylsulfanylphenyl group, a methylsulfinylphenyl group, a methylsulfonylphenyl group, a trifluoromethylsulfanylphenyl group, a trifluoromethylsulfinylphenyl group, and a trifluoromethylsulfonylphenyl group;
as a 5- or 6-membered aromatic heterocyclic group optionally substituted with 0 to 4 substituents C, a pyridyl group, a pyridine-N-oxide group, a 2-fluoropyridyl group, a 3-fluoropyridyl group, a 4-fluoropyridyl group, a 2-chloropyridyl group, a 3-chloropyridyl group, a 4-chloropyridyl group, a 2-bromopyridyl group, a 3-bromopyridyl group, a 4-bromopyridyl group, a 2-iodopyridyl group, a 3-iodopyridyl group, a 4-iodopyridyl group, a 2-methylpyridyl group, a 3-methylpyridyl group, a 4-methylpyridyl group, a 2-trifluoromethylpyridyl group, a 3-trifluoromethylpyridyl group, a 4-trifluoromethylpyridyl group, a 2-cyclopropylpyridyl group, a 3-cyclopropylpyridyl group, a 4-cyclopropylpyridyl group, a 2-methoxypyridyl group, a 3-methoxypyridyl group, a 4-methoxypyridyl group, a pyridazinyl group, a 3-fluoropyridazinyl group, a 4-fluoropyridazinyl group, a 3-chloropyridyldazinyl group, a 4-chloropyridazinyl group, a 3-bromopyridazinyl group, a 4-bromopyridazinyl group, a 3-methylpyridazinyl group, a 4-methylpyridazinyl group, a pyrimidinyl group, a 2-fluoropyrimidinyl group, a 4-fluoropyrimidinyl group, a 5-fluoropyrimidinyl group, a 2-chloropyrimidinyl group, a 4-chloropyrimidinyl group, a 5-chloropyrimidinyl group, a 2-bromopyrimidinyl group, a 4-bromopyrimidinyl group, a 5-bromopyrimidinyl group, a 2-methylpyrimidinyl group, a 4-methylpyrimidinyl group, a 5-methylpyrimidinyl group, a 2-trifluoromethylpyrimidinyl group, a 4-trifluoromethylpyrimidinyl group, a 5-trifluoromethylpyrimidinyl group, a 2-methoxypyrimidinyl group, a 4-methoxypyrimidinyl group, a 5-methoxypyrimidinyl group, a pyrazinyl group, a 2-fluoropyrazinyl group, a 2-chloropyrazinyl group, a 2-bromopyrazinyl group, a 2-methylpyrazinyl group, a 2-trifluoromethylpyrazinyl group, a 2-methoxypyrazinyl group, a pyrazolyl group, a triazolyl group, a 1,2,4-oxadiazolyl group, a (5-trifluoromethyl)-1,2,4-oxadiazolyl group, and a thiazolyl group; and
as a 3- to 6-membered ring group containing one or two oxygen atoms, a tetrahydrofuran-2-yl group, a tetrahydrofuran-3-yl group, a tetrahydro-2H-pyran-2-yl group, a tetrahydro-2H-pyran-3-yl group, a tetrahydro-2H-pyran-4-yl group, a 3-dihydrofuran-2(3H)-one group, a 4-dihydrofuran-2(3H)-one group, a 3-tetrahydro-2H-pyran-2-one group, a 4-tetrahydro-2H-pyran-2-one group, and a 2-oxotetrahydrofuran-3-yl group.

Particularly preferred specific examples of the “substituent A” include a cyano group;
as a halogen atom, a fluorine atom;
as a C3 to C8 cycloalkyl group, a cyclopropyl group;
as a C1 to C6 alkoxy group, a methoxy group and an ethoxy group;
as a phenyl group optionally substituted with 0 to 5 substituents C, a phenyl group, a cyanophenyl group, a fluorophenyl group, a chlorophenyl group, a dichlorophenyl group, a tolyl group, a trifluoromethylphenyl group, a cyclopropylphenyl group, a methoxyphenyl group, a trifluoromethoxyphenyl group, a methylsulfonyiphenyl group, a trifluoromethylsulfanylphenyl group, and a trifluoromethylsulfonylphenyl group;
as a 5- or 6-membered aromatic heterocyclic group optionally substituted with 0 to 4 substituents C, a pyridyl group, a pyridine-N-oxide group, a 2-fluoropyridyl group, a 3-fluoropyridyl group, a 4-fluoropyridyl group, a 2-chloropyridyl group, a 3-chloropyridyl group, a 4-chloropyridyl group, a 2-bromopyridyl group, a 3-bromopyridyl group, a 4-bromopyridyl group, a 2-methylpyridyl group, a 3-methylpyridyl group, a 4-methylpyridyl group, a 2-trifluoromethylpyridyl group, a 3-trifluoromethylpyridyl group, a 4-trifluoromethylpyridyl group, a 2-cyclopropylpyridyl group, a 2-methoxypyridyl group, a 3-methoxypyridyl group, a 4-methoxypyridyl group, a pyridazinyl group, a 3-fluoropyridazinyl group, a 4-fluoropyridazinyl group, a 3-chloropyridyldazinyl group, a 4-chloropyridazinyl group, a 3-methylpyridazinyl group, a 4-methylpyridazinyl group, a pyrimidinyl group, a 2-fluoropyrimidinyl group, a 4-fluoropyrimidinyl group, a 5-fluoropyrimidinyl group, a 2-chloropyrimidinyl group, a 4-chloropyrimidinyl group, a 5-chloropyrimidinyl group, a 2-methylpyrimidinyl group, a 4-methylpyrimidinyl group, a 5-methylpyrimidinyl group, a 2-trifluoromethylpyrimidinyl group, a 4-trifluoromethylpyrimidinyl group, a 5-trifluoromethylpyrimidinyl group, a 2-methoxypyrimidinyl group, a 4-methoxypyrimidinyl group, a 5-methoxypyrimidinyl group, a pyrazinyl group, a 2-fluoropyrazinyl group, a 2-chloropyrazinyl group, a 2-methylpyrazinyl group, a 2-trifluoromethylpyrazinyl group, a 2-methoxypyrazinyl group, a pyrazolyl group, a triazolyl group, a 1,2,4-oxadiazolyl group, a (5-trifluoromethyl)-1,2,4-oxadiazolyl group, and a thiazolyl group; and
as a 3- to 6-membered ring group containing one or two oxygen atoms, a tetrahydrofuran-2-yl group, a tetrahydrofuran-3-yl group, a tetrahydro-2H-pyran-2-yl group, a tetrahydro-2H-pyran-3-yl group, a tetrahydro-2H-pyran-4-yl group, a 3-dihydrofuran-2(3H)-one group, and a 2-oxotetrahydrofuran-3-yl group.

The “substituent B” is selected from the group consisting of a hydroxyl group, a cyano group, a nitro group, a halogen atom, a C1 to C6 alkyl group, a C1 to C6 haloalkyl group, a C3 to C8 cycloalkyl group, a C1 to C6 alkoxy group, a C1 to C6 haloalkoxy group, a C3 to C8 cycloalkoxy group, and Rx5S(O)p- (where Rx5 represents a hydroxyl group, a halogen atom, a C1 to C6 alkyl group, a C1 to C6 haloalkyl group, a C3 to C8 cycloalkyl group, a C2 to C6 alkenyl group, a C2 to C6 haloalkenyl group, a C2 to C6 alkynyl group, or a C2 to C6 haloalkynyl group, and p has the same meaning as above).

Among them, the “substituent B” is selected from the group consisting of a hydroxyl group, a cyano group, a halogen atom, a C1 to C6 alkyl group, a C1 to C6 haloalkyl group, a C3 to C8 cycloalkyl group, a C1 to C6 alkoxy group, a C1 to C6 haloalkoxy group, a C3 to C8 cycloalkoxy group, and Rx5S(O)p- (where Rx5 and p have the same meanings as above).

Particularly, the “substituent B” is selected from the group consisting of a halogen atom, a C1 to C6 alkyl group, a C1 to C6 haloalkyl group, a C1 to C6 alkoxy group, and a C1 to C6 haloalkoxy group.

Preferred specific examples of the “substituent B” include a hydroxyl group; a cyano group; a nitro group;
as a halogen atom, a fluorine atom, a chlorine atom, a bromine atom, and an iodine atom;
as a C1 to C6 alkyl group, a methyl group, an ethyl group, a propyl group, an isopropyl group, a butyl group, an isobutyl group, a sec-butyl group, a t-butyl group, a pentyl group, an isopentyl group, a 2-methylbutyl group, a neopentyl group, a 1-ethylpropyl group, and a hexyl group; as a C1 to C6 haloalkyl group, a difluoromethyl group, a trifluoromethyl group, a dichloromethyl group, a trichloromethyl group, a dibromomethyl group, a 2,2-difluoroethyl group, a 2,2,2-trifluoroethyl group, a 1,1,2,2-tetrafluoroethyl group, a 1,2,2,2-tetrafluoroethyl group, a pentafluoroethyl group, a 2,2,3,3,3-pentafluoropropyl group, and a heptafluoropropyl group;
as a C3 to C8 cycloalkyl group, a cyclopropyl group, a cyclobutyl group, a cyclopentyl group, and a cyclohexyl group;
as a C1 to C6 alkoxy group, a methoxy group, an ethoxy group, a propyloxy group, and an isopropyloxy group;
as a C1 to C6 haloalkoxy group, a difluoromethoxy group, a trifluoromethoxy group, a 2,2-difluoroethoxy group, a 2,2,2-trifluoroethoxy group, a 2,2,3,3-tetrafluoropropyloxy group, and a 2,2,3,3,3-pentafluoropropyloxy group;
as a C3 to C8 cycloalkoxy group, a cyclopropyloxy group, a cyclobutyloxy group, a cyclopentyloxy group, and a cyclohexyloxy group; and as Rx5S(O)p-, a methylsulfanyl group, a methylsulfinyl group, a methylsulfonyl group, an ethylsulfanyl group, an ethylsulfinyl group, an ethylsulfonyl group, a propylsulfanyl group, a propylsulfinyl group, a propylsulfonyl group, a trifluoromethylsulfanyl group, a trifluoromethylsulfinyl group, and a trifluoromethylsulfonyl group.

More preferred specific examples of the “substituent B” include a hydroxyl group; a cyano group;
as a halogen atom, a fluorine atom, a chlorine atom, a bromine atom, and an iodine atom;
as a C1 to C6 alkyl group, a methyl group, an ethyl group, a propyl group, an isopropyl group, a butyl group, an isobutyl group, a sec-butyl group, and a t-butyl group;
as a C1 to C6 haloalkyl group, a difluoromethyl group, a trifluoromethyl group, a 2,2-difluoroethyl group, a 2,2,2-trifluoroethyl group, a 1,1,2,2-tetrafluoroethyl group, a pentafluoroethyl group, and a heptafluoropropyl group;
as a C3 to C8 cycloalkyl group, a cyclopropyl group;
as a C1 to C6 alkoxy group, a methoxy group, an ethoxy group, a propyloxy group, and an isopropyloxy group;
as a C1 to C6 haloalkoxy group, a trifluoromethoxy group and a 2,2,2-trifluoroethoxy group;
as a C3 to C8 cycloalkoxy group, a cyclopropyloxy group and a cyclopentyloxy group; and
as Rx5S(O)p-, a methylsulfanyl group, a methylsulfinyl group, a methylsulfonyl group, an ethylsulfanyl group, an ethylsulfinyl group, an ethylsulfonyl group, a trifluoromethylsulfanyl group, a trifluoromethylsulfinyl group, and a trifluoromethylsulfonyl group.

Still more preferred specific examples of the “substituent B” include a cyano group;
as a halogen atom, a fluorine atom, a chlorine atom, a bromine atom, and an iodine atom;
as a C1 to C6 alkyl group, a methyl group, an ethyl group, a propyl group, and an isopropyl group;
as a C1 to C6 haloalkyl group, a difluoromethyl group, a trifluoromethyl group, a pentafluoroethyl group, and a heptafluoropropyl group;
as a C1 to C6 alkoxy group, a methoxy group, an ethoxy group, and a propyloxy group;
as a C1 to C6 haloalkoxy group, a trifluoromethoxy group; and
as Rx5S(O)p-, a methylsulfanyl group, a methylsulfinyl group, a methylsulfonyl group, a trifluoromethylsulfanyl group, a trifluoromethylsulfinyl group, and a trifluoromethylsulfonyl group.

The “substituent C” is selected from the group consisting of a cyano group, a halogen atom, a C1 to C6 alkyl group, a C1 to C6 haloalkyl group, a C3 to C8 cycloalkyl group, a C1 to C6 alkoxy group, a C1 to C6 haloalkoxy group, a C3 to C8 cycloalkoxy group, and Rx5S(O)p- (where Rx5 and p have the same meanings as above).

Among them, the “substituent C” is selected from the group consisting of a cyano group, a halogen atom, a C1 to C6 alkyl group, a C1 to C6 haloalkyl group, a C3 to C8 cycloalkyl group, a C1 to C6 alkoxy group, and a C1 to C6 haloalkoxy group.

Particularly, the “substituent C” is selected from the group consisting of a cyano group, a halogen atom, a C1 to C6 alkyl group, a C1 to C6 haloalkyl group, and a C1 to C6 alkoxy group.

Preferred specific examples of the “substituent C” include a cyano group;
as a halogen atom, a fluorine atom, a chlorine atom, a bromine atom, and an iodine atom;
as a C1 to C6 alkyl group, a methyl group, an ethyl group, a propyl group, an isopropyl group, a butyl group, an isobutyl group, a sec-butyl group, a t-butyl group, a pentyl group, an isopentyl group, a 2-methylbutyl group, a neopentyl group, a 1-ethylpropyl group, and a hexyl group; as a C1 to C6 haloalkyl group, a difluoromethyl group, a trifluoromethyl group, a dichloromethyl group, a trichloromethyl group, a dibromomethyl group, a 2,2-difluoroethyl group, a 2,2,2-trifluoroethyl group, a 1,1,2,2-tetrafluoroethyl group, a 1,2,2,2-tetrafluoroethyl group, a pentafluoroethyl group, a 2,2,3,3,3-pentafluoropropyl group, and a heptafluoropropyl group;
as a C3 to C8 cycloalkyl group, a cyclopropyl group, a cyclobutyl group, a cyclopentyl group, and a cyclohexyl group;
as a C1 to C6 alkoxy group, a methoxy group, an ethoxy group, a propyloxy group, and an isopropyloxy group;
as a C1 to C6 haloalkoxy group, a difluoromethoxy group, a trifluoromethoxy group, a 2,2-difluoroethoxy group, a 2,2,2-trifluoroethoxy group, a 2,2,3,3-tetrafluoropropyloxy group, and a 2,2,3,3,3-pentafluoropropyloxy group;
as a C3 to C8 cycloalkoxy group, a cyclopropyloxy group, a cyclobutyloxy group, a cyclopentyloxy group, and a cyclohexyloxy group; and
as Rx5S(O)p-, a methylsulfanyl group, a methylsulfinyl group, a methylsulfonyl group, an ethylsulfanyl group, an ethylsulfinyl group, an ethylsulfonyl group, a propylsulfanyl group, a propylsulfinyl group, a propylsulfonyl group, a trifluoromethylsulfanyl group, a trifluoromethylsulfinyl group, and a trifluoromethylsulfonyl group.

More preferred specific examples of the “substituent C” include a cyano group;
as a halogen atom, a fluorine atom, a chlorine atom, a bromine atom, and an iodine atom;
as a C1 to C6 alkyl group, a methyl group, an ethyl group, a propyl group, an isopropyl group, a butyl group, an isobutyl group, a sec-butyl group, and a t-butyl group;
as a C1 to C6 haloalkyl group, a difluoromethyl group, a trifluoromethyl group, a 2,2-difluoroethyl group, a 2,2,2-trifluoroethyl group, a 1,1,2,2-tetrafluoroethyl group, a pentafluoroethyl group, and a heptafluoropropyl group;
as a C3 to C8 cycloalkyl group, a cyclopropyl group;
as a C1 to C6 alkoxy group, a methoxy group, an ethoxy group, a propyloxy group, and an isopropyloxy group;
as a C1 to C6 haloalkoxy group, a trifluoromethoxy group and a 2,2,2-trifluoroethoxy group;
as a C3 to C8 cycloalkoxy group, a cyclopropyloxy group and a cyclopentyloxy group; and
as Rx5S(O)p-, a methylsulfanyl group, a methylsulfinyl group, a methylsulfonyl group, an ethylsulfanyl group, an ethylsulfinyl group, an ethylsulfonyl group, a trifluoromethylsulfanyl group, a trifluoromethylsulfinyl group, and a trifluoromethylsulfonyl group.

Still more preferred specific examples of the “substituent C” include a cyano group;
as a halogen atom, a fluorine atom, a chlorine atom, a bromine atom, and an iodine atom;
as a C1 to C6 alkyl group, a methyl group, an ethyl group, a propyl group, and an isopropyl group;
as a C1 to C6 haloalkyl group, a difluoromethyl group, a trifluoromethyl group, a pentafluoroethyl group, and a heptafluoropropyl group;
as a C1 to C6 alkoxy group, a methoxy group, an ethoxy group, and a propyloxy group; and
as Rx5S(O)p-, a methylsulfanyl group, a methylsulfinyl group, a methylsulfonyl group, a trifluoromethylsulfanyl group, a trifluoromethylsulfinyl group, and a trifluoromethylsulfonyl group.

The compounds represented by Formula (1-1), Formula (1-2), Formula (1-3), and Formula (1-4) may contain one or more asymmetric atoms. An isomer ratio in this case is a mixing ratio of individual or arbitrary proportions, and is not particularly limited.

The compounds represented by Formula (1-1), Formula (1-2), Formula (1-3), and Formula (1-4) may contain one or two axial asymmetries. An isomer ratio in this case is a mixing ratio of individual or arbitrary proportions, and is not particularly limited.

The compounds represented by Formula (1-1), Formula (1-2), Formula (1-3), and Formula (1-4) may contain a geometric isomer. An isomer ratio in this case is a mixing ratio of individual or arbitrary proportions, and is not particularly limited.

The compounds represented by Formula (1-1), Formula (1-2), Formula (1-3), and Formula (1-4) may contain a rotamer. An isomer ratio in this case is a mixing ratio of individual or arbitrary proportions, and is not particularly limited.

The compounds represented by Formula (1-1), Formula (1-2), Formula (1-3), and Formula (1-4) may be capable of forming salts. Examples thereof include acid salts such as hydrochloric acid, sulfuric acid, acetic acid, fumaric acid, and maleic acid, and metal salts such as sodium, potassium, and calcium, but are not limited thereto. A salt that can be used as an arthropod pest control agent for agriculture and horticulture is preferable.

The ranges of all compounds obtained by arbitrarily combining the preferred ranges for R1, R2, R3, R4, R5, Het, G, R6, R7, R8, R9, the substituent A, the substituent B, the substituent C, Rx1, Rx2, Rx3, Rx4, Rx5, and p described above are also described as the ranges of Formula (1-1) or Formula (1-2), or its production intermediate compound Formula (1-3) or Formula (1-4) of the present invention.

Specific compounds of the present invention may be represented by a combination of Structural Formulas C-1 to C-224 (where the binding site cut by the wavy line represents a site where Het in Table 2 is bound) representing Het (where Het has the same meaning as above) shown in Table 1, Structural Formulas Pyra-1 to Pyra-48 (where in Table 2 R1, R2, Het, and p have the same meanings as above, and Z represents —NR3R4) shown in Table 2, substituents R1-1 to R1-203 and R1-500 to R1-1315 (where, in the structural formula of Table 3, the binding site cut by the wavy line represents a site where R1 in Table 2 is bound) representing R1 (where R1 has the same meaning as above) shown in Table 3, and substituents R2-1 to R2-203 and R2-500 to R2-1315 (where, in Table 3, the binding site cut by the wavy line represents a site where R2 in Table 2 is bound) representing R2 (where R2 has the same meaning as above), and substituents Z-1 to Z-6414 representing Z (where Z represents —NR3R4) shown in Table 4. These compounds are for illustrative purposes and the present invention is not limited thereto.

TABLE 1

| | |
|---|---|
| 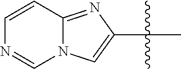 [SMILES: *c1cn2cnccc2n1] | C-1 |
| 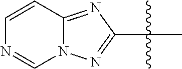 [SMILES: *c1nc2ccncn2n1] | C-2 |
| 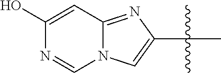 [SMILES: *c1cn2cnc(O)cc2n1] | C-3 |
| 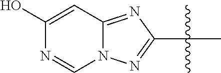 [SMILES: *c1nc2cc(O)ncn2n1] | C-4 |
| 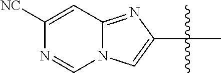 [SMILES: *c1cn2cnc(C#N)cc2n1] | C-5 |
| 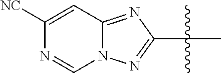 [SMILES: *c1nc2cc(C#N)ncn2n1] | C-6 |
| 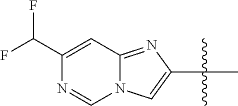 [SMILES: *c1cn2cnc(C(F)F)cc2n1] | C-7 |
| 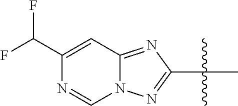 [SMILES: *c1nc2cc(C(F)F)ncn2n1] | C-8 |
| 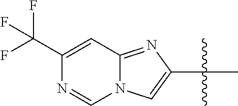 [SMILES: *c1cn2cnc(C(F)(F)F)cc2n1] | C-9 |
| 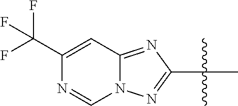 [SMILES: *c1nc2cc(C(F)(F)F)ncn2n1] | C-10 |
| 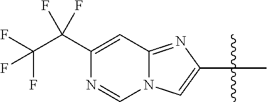 [SMILES: *c1cn2cnc(C(F)(F)C(F)(F)F)cc2n1] | C-11 |
| 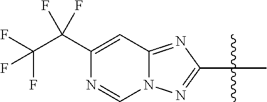 [SMILES: *c1nc2cc(C(F)(F)C(F)(F)F)ncn2n1] | C-12 |
| 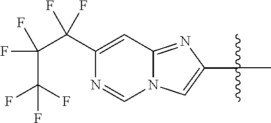 [SMILES: *c1cn2cnc(C(F)(F)C(F)(F)C(F)(F)F)cc2n1] | C-13 |
| 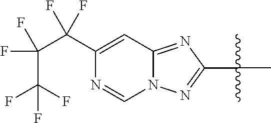 [SMILES: *c1nc2cc(C(F)(F)C(F)(F)C(F)(F)F)ncn2n1] | C-14 |
| 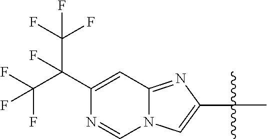 [SMILES: *c1cn2cnc(C(F)(C(F)(F)F)C(F)(F)F)cc2n1] | C-15 |
| 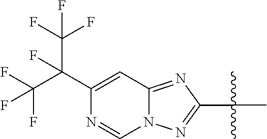 [SMILES: *c1nc2cc(C(F)(C(F)(F)F)C(F)(F)F)ncn2n1] | C-16 |
| 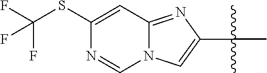 [SMILES: *c1cn2cnc(SC(F)(F)F)cc2n1] | C-17 |
| 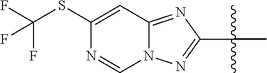 [SMILES: *c1nc2cc(SC(F)(F)F)ncn2n1] | C-18 |
| 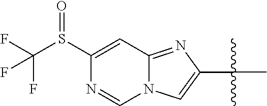 [SMILES: *c1cn2cnc(S(=O)C(F)(F)F)cc2n1] | C-19 |
| 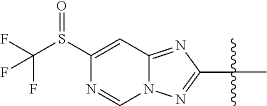 [SMILES: *c1nc2cc(S(=O)C(F)(F)F)ncn2n1] | C-20 |
| 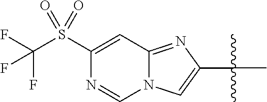 [SMILES: *c1cn2cnc(S(=O)(=O)C(F)(F)F)cc2n1] | C-21 |
| 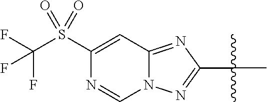 [SMILES: *c1nc2cc(S(=O)(=O)C(F)(F)F)ncn2n1] | C-22 |
| 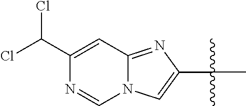 [SMILES: *c1cn2cnc(C(Cl)Cl)cc2n1] | C-23 |
| 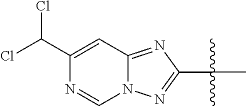 [SMILES: *c1nc2cc(C(Cl)Cl)ncn2n1] | C-24 |
| 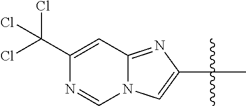 [SMILES: *c1cn2cnc(C(Cl)(Cl)Cl)cc2n1] | C-25 |
| 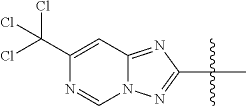 [SMILES: *c1nc2cc(C(Cl)(Cl)Cl)ncn2n1] | C-26 |
| 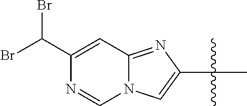 [SMILES: *c1cn2cnc(C(Br)Br)cc2n1] | C-27 |
| 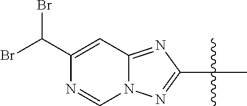 [SMILES: *c1nc2cc(C(Br)Br)ncn2n1] | C-28 |
| 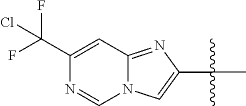 [SMILES: *c1cn2cnc(C(F)(F)Cl)cc2n1] | C-29 |
| 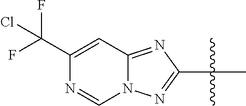 [SMILES: *c1nc2cc(C(F)(F)Cl)ncn2n1] | C-30 |
| 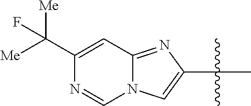 [SMILES: *c1cn2cnc(C(C)(C)F)cc2n1] | C-31 |
| 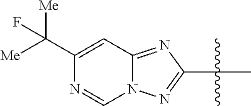 [SMILES: *c1nc2cc(C(C)(C)F)ncn2n1] | C-32 |
| 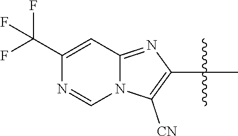 [SMILES: *c1nc2cc(C(F)(F)F)ncn2c1C#N] | C-33 |
| 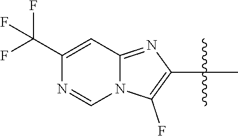 [SMILES: *c1nc2cc(C(F)(F)F)ncn2c1F] | C-34 |
| 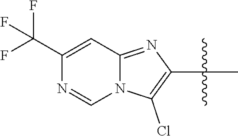 [SMILES: *c1nc2cc(C(F)(F)F)ncn2c1Cl] | C-35 |
| 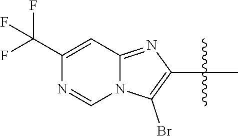 [SMILES: *c1nc2cc(C(F)(F)F)ncn2c1Br] | C-36 |
| 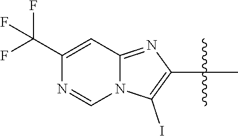 [SMILES: *c1nc2cc(C(F)(F)F)ncn2c1I] | C-37 |
| 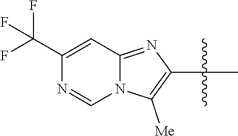 [SMILES: *c1nc2cc(C(F)(F)F)ncn2c1C] | C-38 |
| 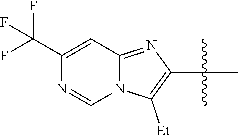 [SMILES: *c1nc2cc(C(F)(F)F)ncn2c1CC] | C-39 |
| 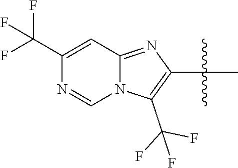 [SMILES: *c1nc2cc(C(F)(F)F)ncn2c1C(F)(F)F] | C-40 |
| 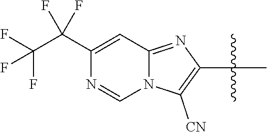 [SMILES: *c1nc2cc(C(F)(F)C(F)(F)F)ncn2c1C#N] | C-41 |
| 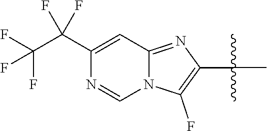 [SMILES: *c1nc2cc(C(F)(F)C(F)(F)F)ncn2c1F] | C-42 |
| 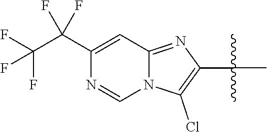 [SMILES: *c1nc2cc(C(F)(F)C(F)(F)F)ncn2c1Cl] | C-43 |
| 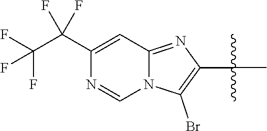 [SMILES: *c1nc2cc(C(F)(F)C(F)(F)F)ncn2c1Br] | C-44 |
| 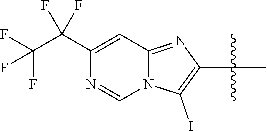 [SMILES: *c1nc2cc(C(F)(F)C(F)(F)F)ncn2c1I] | C-45 |
| 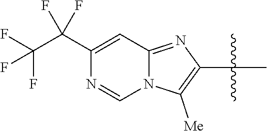 [SMILES: *c1nc2cc(C(F)(F)C(F)(F)F)ncn2c1C] | C-46 |
| 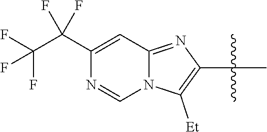 [SMILES: *c1nc2cc(C(F)(F)C(F)(F)F)ncn2c1CC] | C-47 |
| 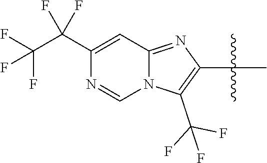 [SMILES: *c1nc2cc(C(F)(F)C(F)(F)F)ncn2c1C(F)(F)F] | C-48 |
| 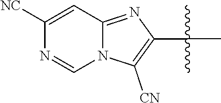 [SMILES: *c1nc2cc(C#N)ncn2c1C#N] | C-49 |
| 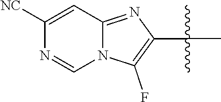 [SMILES: *c1nc2cc(C#N)ncn2c1F] | C-50 |
| 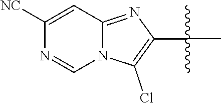 [SMILES: *c1nc2cc(C#N)ncn2c1Cl] | C-51 |
| 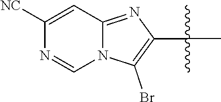 [SMILES: *c1nc2cc(C#N)ncn2c1Br] | C-52 |
| 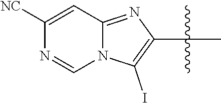 [SMILES: *c1nc2cc(C#N)ncn2c1I] | C-53 |
| 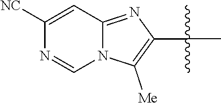 [SMILES: *c1nc2cc(C#N)ncn2c1C] | C-54 |
| 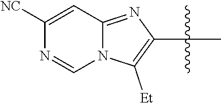 [SMILES: *c1nc2cc(C#N)ncn2c1CC] | C-55 |
| 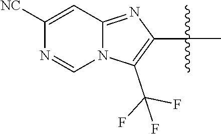 [SMILES: *c1nc2cc(C#N)ncn2c1C(F)(F)F] | C-56 |
| 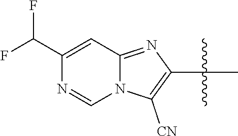 [SMILES: *c1nc2cc(C(F)F)ncn2c1C#N] | C-57 |
| 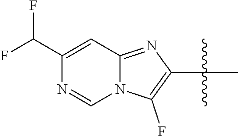 [SMILES: *c1nc2cc(C(F)F)ncn2c1F] | C-58 |
| 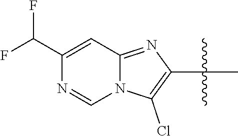 [SMILES: *c1nc2cc(C(F)F)ncn2c1Cl] | C-59 |
| 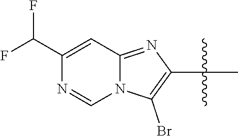 [SMILES: *c1nc2cc(C(F)F)ncn2c1Br] | C-60 |
| 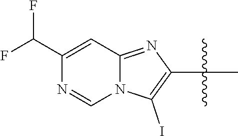 [SMILES: *c1nc2cc(C(F)F)ncn2c1I] | C-61 |
| 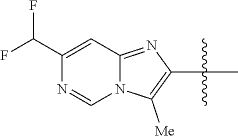 [SMILES: *c1nc2cc(C(F)F)ncn2c1C] | C-62 |
| 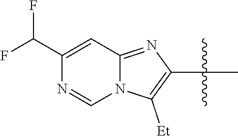 [SMILES: *c1nc2cc(C(F)F)ncn2c1CC] | C-63 |
| 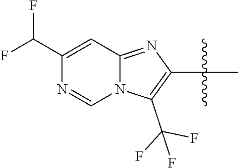 [SMILES: *c1nc2cc(C(F)F)ncn2c1C(F)(F)F] | C-64 |
| 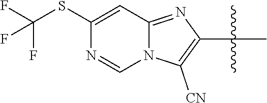 [SMILES: *c1nc2cc(SC(F)(F)F)ncn2c1C#N] | C-65 |
| 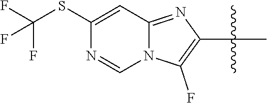 [SMILES: *c1nc2cc(SC(F)(F)F)ncn2c1F] | C-66 |
| 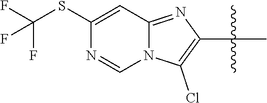 [SMILES: *c1nc2cc(SC(F)(F)F)ncn2c1Cl] | C-67 |
| 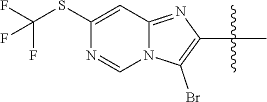 [SMILES: *c1nc2cc(SC(F)(F)F)ncn2c1Br] | C-68 |
| 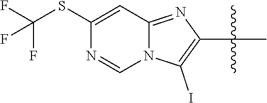 [SMILES: *c1nc2cc(SC(F)(F)F)ncn2c1I] | C-69 |
| 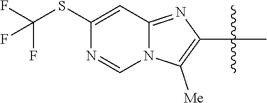 [SMILES: *c1nc2cc(SC(F)(F)F)ncn2c1C] | C-70 |
| 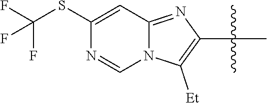 [SMILES: *c1nc2cc(SC(F)(F)F)ncn2c1CC] | C-71 |
| 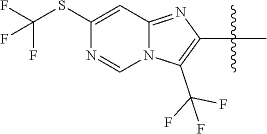 [SMILES: *c1nc2cc(SC(F)(F)F)ncn2c1C(F)(F)F] | C-72 |
| 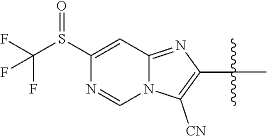 [SMILES: *c1nc2cc(S(=O)C(F)(F)F)ncn2c1C#N] | C-73 |
| 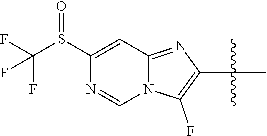 [SMILES: *c1nc2cc(S(=O)C(F)(F)F)ncn2c1F] | C-74 |
| 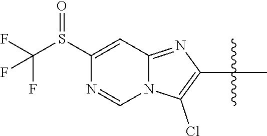 [SMILES: *c1nc2cc(S(=O)C(F)(F)F)ncn2c1Cl] | C-75 |
| 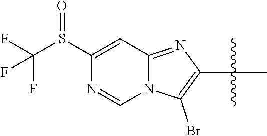 [SMILES: *c1nc2cc(S(=O)C(F)(F)F)ncn2c1Br] | C-76 |
| 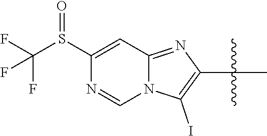 [SMILES: *c1nc2cc(S(=O)C(F)(F)F)ncn2c1I] | C-77 |
| 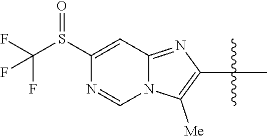 [SMILES: *c1nc2cc(S(=O)C(F)(F)F)ncn2c1C] | C-78 |
| 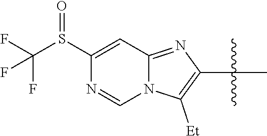 [SMILES: *c1nc2cc(S(=O)C(F)(F)F)ncn2c1CC] | C-79 |
| 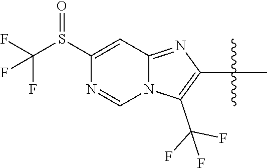 [SMILES: *c1nc2cc(S(=O)C(F)(F)F)ncn2c1C(F)(F)F] | C-80 |
| 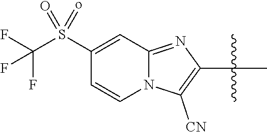 | C-81 |
| 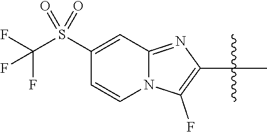 [SMILES: *c1nc2cc(S(=O)(=O)C(F)(F)F)ccn2c1F] | C-82 |
| 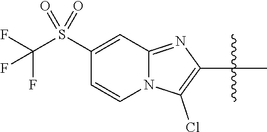 [SMILES: *c1nc2cc(S(=O)(=O)C(F)(F)F)ccn2c1Cl] | C-83 |
| 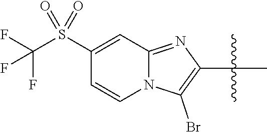 [SMILES: *c1nc2cc(S(=O)(=O)C(F)(F)F)ccn2c1Br] | C-84 |
| 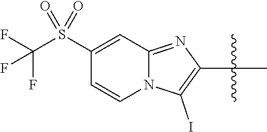 [SMILES: *c1nc2cc(S(=O)(=O)C(F)(F)F)ccn2c1I] | C-85 |
| 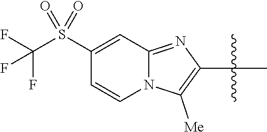 [SMILES: *c1nc2cc(S(=O)(=O)C(F)(F)F)ccn2c1C] | C-86 |
| 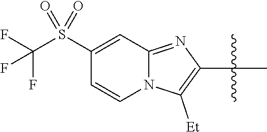 [SMILES: *c1nc2cc(S(=O)(=O)C(F)(F)F)ccn2c1CC] | C-87 |
| 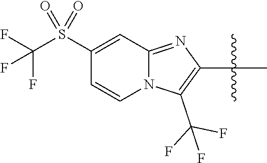 [SMILES: *c1nc2cc(S(=O)(=O)C(F)(F)F)ccn2c1C(F)(F)F] | C-88 |
| 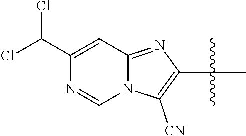 [SMILES: *c1nc2cc(C(Cl)Cl)ncn2c1C#N] | C-89 |
| 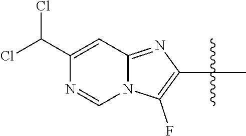 [SMILES: *c1nc2cc(C(Cl)Cl)ncn2c1F] | C-90 |
| 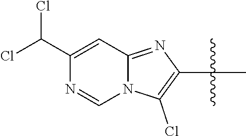 [SMILES: *c1nc2cc(C(Cl)Cl)ncn2c1Cl] | C-91 |
| 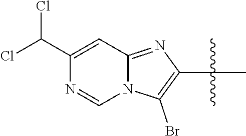 [SMILES: *c1nc2cc(C(Cl)Cl)ncn2c1Br] | C-92 |
| 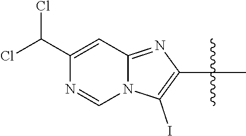 [SMILES: *c1nc2cc(C(Cl)Cl)ncn2c1I] | C-93 |
| 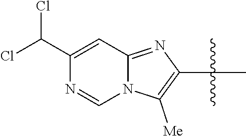 [SMILES: *c1nc2cc(C(Cl)Cl)ncn2c1C] | C-94 |
| 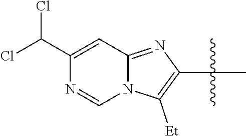 [SMILES: *c1nc2cc(C(Cl)Cl)ncn2c1CC] | C-95 |
| 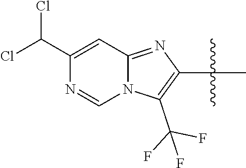 [SMILES: *c1nc2cc(C(Cl)Cl)ncn2c1C(F)(F)F] | C-96 |
| 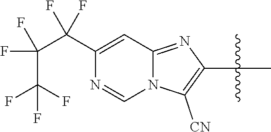 [SMILES: *c1nc2cc(C(F)(F)C(F)(F)C(F)(F)F)ncn2c1C#N] | C-97 |
| 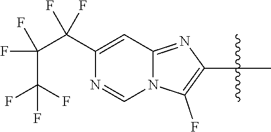 [SMILES: *c1nc2cc(C(F)(F)C(F)(F)C(F)(F)F)ncn2c1F] | C-98 |
| 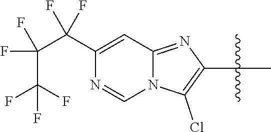 [SMILES: *c1nc2cc(C(F)(F)C(F)(F)C(F)(F)F)ncn2c1Cl] | C-99 |
| 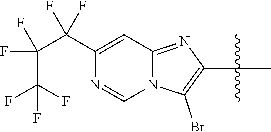 [SMILES: *c1nc2cc(C(F)(F)C(F)(F)C(F)(F)F)ncn2c1Br] | C-100 |
| 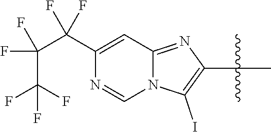 [SMILES: *c1nc2cc(C(F)(F)C(F)(F)C(F)(F)F)ncn2c1I] | C-101 |
| 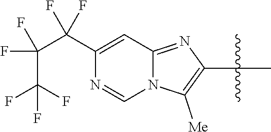 [SMILES: *c1nc2cc(C(F)(F)C(F)(F)C(F)(F)F)ncn2c1C] | C-102 |
| 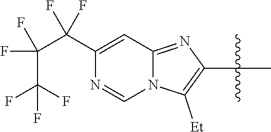 [SMILES: *c1nc2cc(C(F)(F)C(F)(F)C(F)(F)F)ncn2c1CC] | C-103 |
| 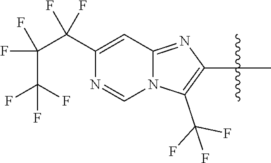 [SMILES: *c1nc2cc(C(F)(F)C(F)(F)C(F)(F)F)ncn2c1C(F)(F)F] | C-104 |
| 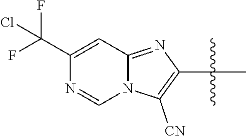 [SMILES: *c1nc2cc(C(F)(F)Cl)ncn2c1C#N] | C-105 |
| 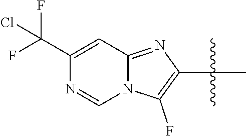 [SMILES: *c1nc2cc(C(F)(F)Cl)ncn2c1F] | C-106 |
| 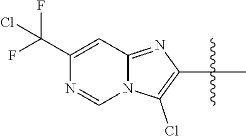 [SMILES: *c1nc2cc(C(F)(F)Cl)ncn2c1Cl] | C-107 |
| 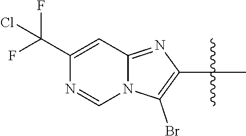 [SMILES: *c1nc2cc(C(F)(F)Cl)ncn2c1Br] | C-108 |
| 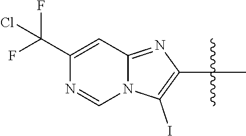 [SMILES: *c1nc2cc(C(F)(F)Cl)ncn2c1I] | C-109 |
| 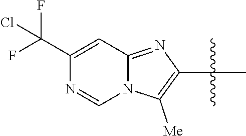 [SMILES: *c1nc2cc(C(F)(F)Cl)ncn2c1C] | C-110 |
| 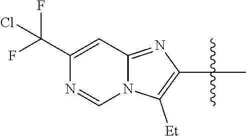 [SMILES: *c1nc2cc(C(F)(F)Cl)ncn2c1CC] | C-111 |
| 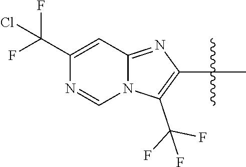 [SMILES: *c1nc2cc(C(F)(F)Cl)ncn2c1C(F)(F)F] | C-112 |
| 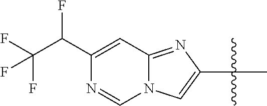 [SMILES: *c1cn2cnc(C(F)C(F)(F)F)cc2n1] | C-113 |
| 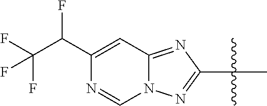 [SMILES: *c1nc2cc(C(F)C(F)(F)F)ncn2n1] | C-114 |
| 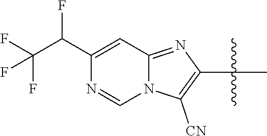 [SMILES: *c1nc2cc(C(F)C(F)(F)F)ncn2c1C#N] | C-115 |
| 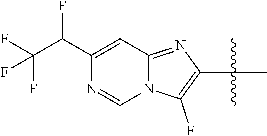 [SMILES: *c1nc2cc(C(F)C(F)(F)F)ncn2c1F] | C-116 |
| 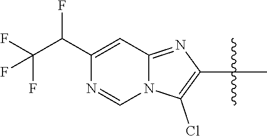 [SMILES: *c1nc2cc(C(F)C(F)(F)F)ncn2c1Cl] | C-117 |
| 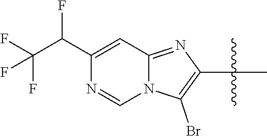 [SMILES: *c1nc2cc(C(F)C(F)(F)F)ncn2c1Br] | C-118 |
| 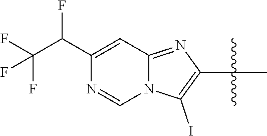 [SMILES: *c1nc2cc(C(F)C(F)(F)F)ncn2c1I] | C-119 |
| 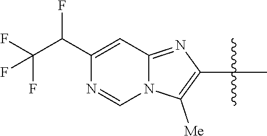 [SMILES: *c1nc2cc(C(F)C(F)(F)F)ncn2c1C] | C-120 |
| 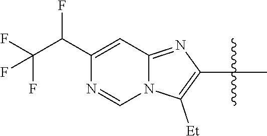 [SMILES: *c1nc2cc(C(F)C(F)(F)F)ncn2c1CC] | C-121 |
| 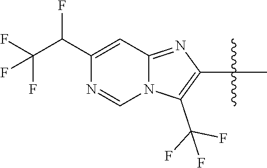 [SMILES: *c1nc2cc(C(F)C(F)(F)F)ncn2c1C(F)(F)F] | C-122 |
| 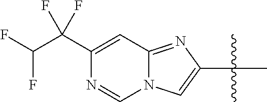 [SMILES: *c1cn2cnc(C(F)(F)C(F)F)cc2n1] | C-123 |
| 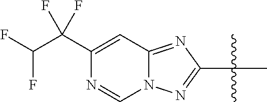 [SMILES: *c1nc2cc(C(F)(F)C(F)F)ncn2n1] | C-124 |
| 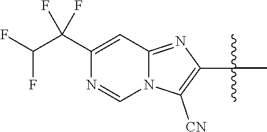 [SMILES: *c1nc2cc(C(F)(F)C(F)F)ncn2c1C#N] | C-125 |
| 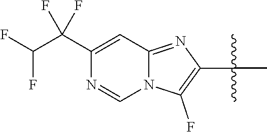 [SMILES: *c1nc2cc(C(F)(F)C(F)F)ncn2c1F] | C-126 |
| 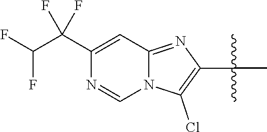 [SMILES: *c1nc2cc(C(F)(F)C(F)F)ncn2c1Cl] | C-127 |
| 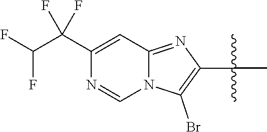 [SMILES: *c1nc2cc(C(F)(F)C(F)F)ncn2c1Br] | C-128 |
| 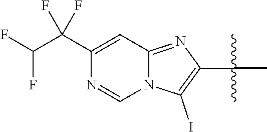 [SMILES: *c1nc2cc(C(F)(F)C(F)F)ncn2c1I] | C-129 |
| 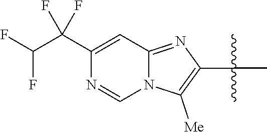 [SMILES: *c1nc2cc(C(F)(F)C(F)F)ncn2c1C] | C-130 |
| 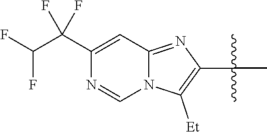 [SMILES: *c1nc2cc(C(F)(F)C(F)F)ncn2c1CC] | C-131 |
| 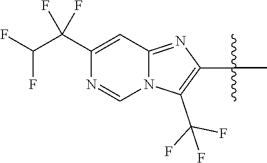 [SMILES: *c1nc2cc(C(F)(F)C(F)F)ncn2c1C(F)(F)F] | C-132 |
| 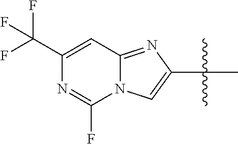 [SMILES: *c1cn2c(F)nc(C(F)(F)F)cc2n1] | C-133 |
| 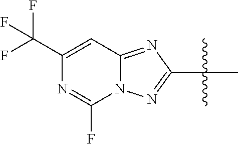 [SMILES: *c1nc2cc(C(F)(F)F)nc(F)n2n1] | C-134 |
| 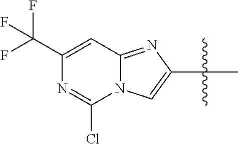 [SMILES: *c1cn2c(Cl)nc(C(F)(F)F)cc2n1] | C-135 |
| 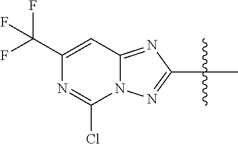 [SMILES: *c1nc2cc(C(F)(F)F)nc(Cl)n2n1] | C-136 |
| 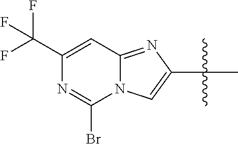 [SMILES: *c1cn2c(Br)nc(C(F)(F)F)cc2n1] | C-137 |
| 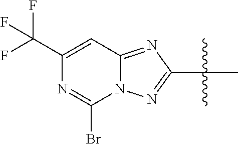 [SMILES: *c1nc2cc(C(F)(F)F)nc(Br)n2n1] | C-138 |
| 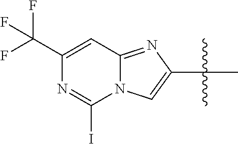 [SMILES: *c1cn2c(I)nc(C(F)(F)F)cc2n1] | C-139 |
| 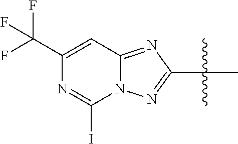 [SMILES: *c1nc2cc(C(F)(F)F)nc(I)n2n1] | C-140 |
| 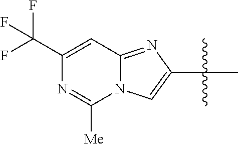 [SMILES: *c1cn2c(C)nc(C(F)(F)F)cc2n1] | C-141 |
| 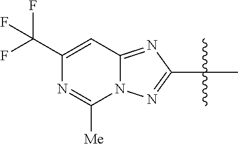 [SMILES: *c1nc2cc(C(F)(F)F)nc(C)n2n1] | C-142 |
| 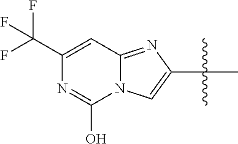 [SMILES: *c1cn2c(O)nc(C(F)(F)F)cc2n1] | C-143 |
| 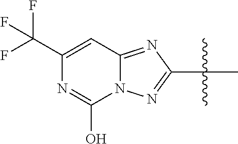 [SMILES: *c1nc2cc(C(F)(F)F)nc(O)n2n1] | C-144 |
| 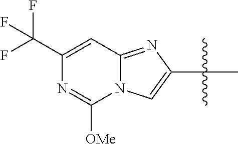 [SMILES: *c1cn2c(OC)nc(C(F)(F)F)cc2n1] | C-145 |
| 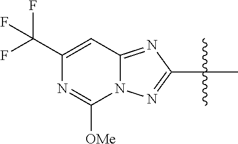 [SMILES: *c1nc2cc(C(F)(F)F)nc(OC)n2n1] | C-146 |
| 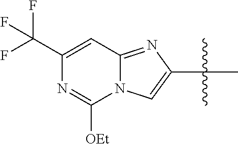 [SMILES: *c1cn2c(OCC)nc(C(F)(F)F)cc2n1] | C-147 |
| 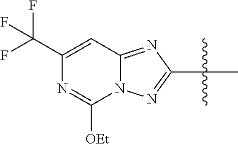 [SMILES: *c1nc2cc(C(F)(F)F)nc(OCC)n2n1] | C-148 |
| 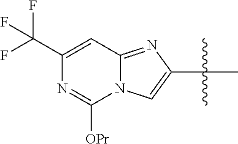 | C-149 |
| 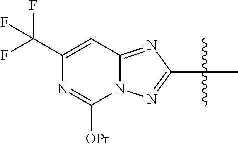 | C-150 |
| 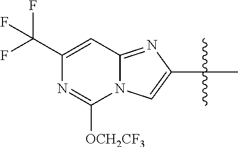 | C-151 |
| 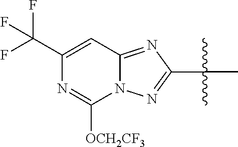 | C-152 |
| 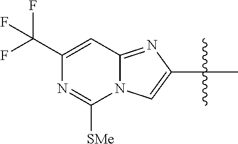 [SMILES: *c1cn2c(SC)nc(C(F)(F)F)cc2n1] | C-153 |
| 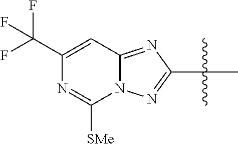 [SMILES: *c1nc2cc(C(F)(F)F)nc(SC)n2n1] | C-154 |
| 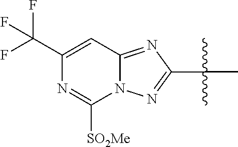 [SMILES: *c1nc2cc(C(F)(F)F)nc(S(C)(=O)=O)n2n1] | C-155 |
| 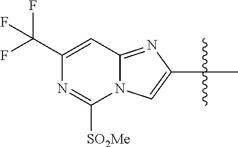 [SMILES: *c1cn2c(S(C)(=O)=O)nc(C(F)(F)F)cc2n1] | C-156 |
| 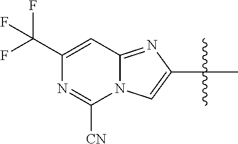 [SMILES: *c1cn2c(C#N)nc(C(F)(F)F)cc2n1] | C-157 |
| 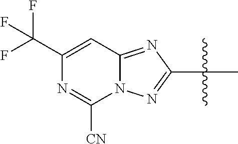 [SMILES: *c1nc2cc(C(F)(F)F)nc(C#N)n2n1] | C-158 |
| 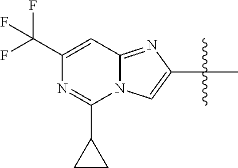 [SMILES: *c1cn2c(C3CC3)nc(C(F)(F)F)cc2n1] | C-159 |
| 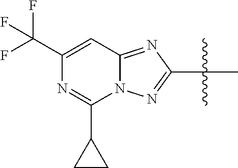 [SMILES: *c1nc2cc(C(F)(F)F)nc(C3CC3)n2n1] | C-160 |
| 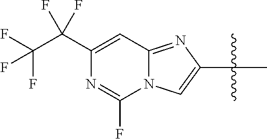 [SMILES: *c1cn2c(F)nc(C(F)(F)C(F)(F)F)cc2n1] | C-161 |
| 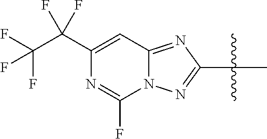 [SMILES: *c1nc2cc(C(F)(F)C(F)(F)F)nc(F)n2n1] | C-162 |
| 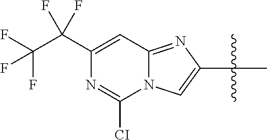 | C-163 |
| 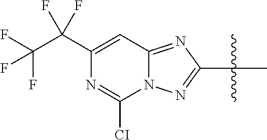 | C-164 |
| 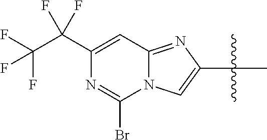 [SMILES: *c1cn2c(Br)nc(C(F)(F)C(F)(F)F)cc2n1] | C-165 |
| 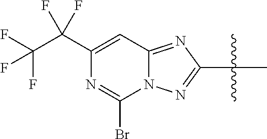 [SMILES: *c1nc2cc(C(F)(F)C(F)(F)F)nc(Br)n2n1] | C-166 |
| 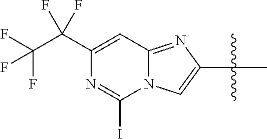 [SMILES: *c1cn2c(I)nc(C(F)(F)C(F)(F)F)cc2n1] | C-167 |
| 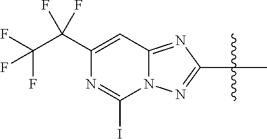 [SMILES: *c1nc2cc(C(F)(F)C(F)(F)F)nc(I)n2n1] | C-168 |
| 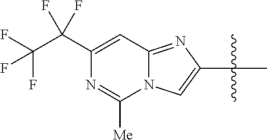 [SMILES: *c1cn2c(C)nc(C(F)(F)C(F)(F)F)cc2n1] | C-169 |
| 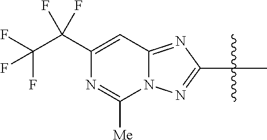 [SMILES: *c1nc2cc(C(F)(F)C(F)(F)F)nc(C)n2n1] | C-170 |
| 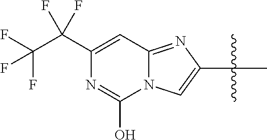 [SMILES: *c1cn2c(O)nc(C(F)(F)C(F)(F)F)cc2n1] | C-171 |
| 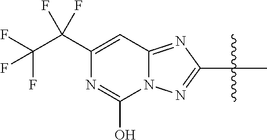 [SMILES: *c1nc2cc(C(F)(F)C(F)(F)F)nc(O)n2n1] | C-172 |
| 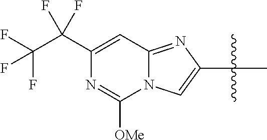 [SMILES: *c1cn2c(OC)nc(C(F)(F)C(F)(F)F)cc2n1] | C-173 |
| 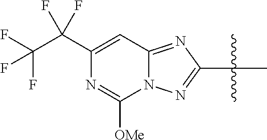 [SMILES: *c1nc2cc(C(F)(F)C(F)(F)F)nc(OC)n2n1] | C-174 |
| 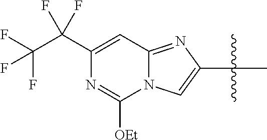 [SMILES: *c1cn2c(OCC)nc(C(F)(F)C(F)(F)F)cc2n1] | C-175 |
| 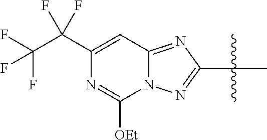 [SMILES: *c1nc2cc(C(F)(F)C(F)(F)F)nc(OCC)n2n1] | C-176 |
| 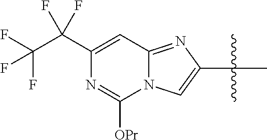 | C-177 |
| 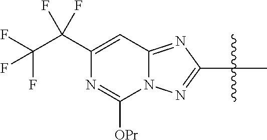 | C-178 |
| 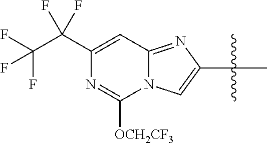 | C-179 |
| 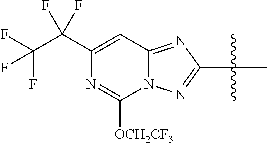 | C-180 |
| 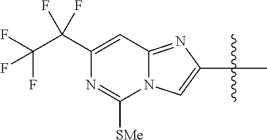 [SMILES: *c1cn2c(SC)nc(C(F)(F)C(F)(F)F)cc2n1] | C-181 |
| 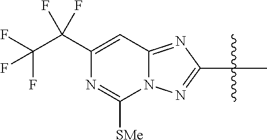 [SMILES: *c1nc2cc(C(F)(F)C(F)(F)F)nc(SC)n2n1] | C-182 |
| 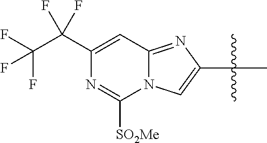 [SMILES: *c1cn2c(S(C)(=O)=O)nc(C(F)(F)C(F)(F)F)cc2n1] | C-183 |
| 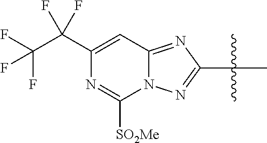 [SMILES: *c1nc2cc(C(F)(F)C(F)(F)F)nc(S(C)(=O)=O)n2n1] | C-184 |
| 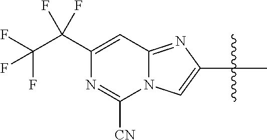 [SMILES: *c1cn2c(C#N)nc(C(F)(F)C(F)(F)F)cc2n1] | C-185 |
| 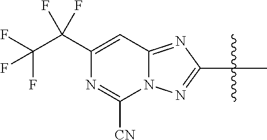 [SMILES: *c1nc2cc(C(F)(F)C(F)(F)F)nc(C#N)n2n1] | C-186 |
| 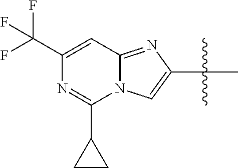 [SMILES: *c1cn2c(C3CC3)nc(C(F)(F)F)cc2n1] | C-187 |
| 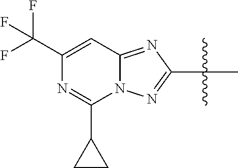 [SMILES: *c1nc2cc(C(F)(F)F)nc(C3CC3)n2n1] | C-188 |
| 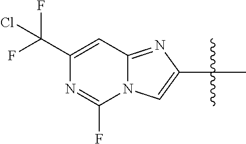 [SMILES: *c1cn2c(F)nc(C(F)(F)Cl)cc2n1] | C-189 |
| 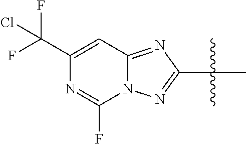 [SMILES: *c1nc2cc(C(F)(F)Cl)nc(F)n2n1] | C-190 |
| 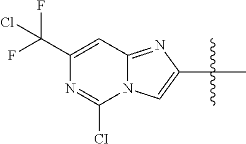 | C-191 |
| 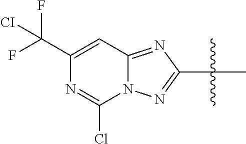 | C-192 |
| 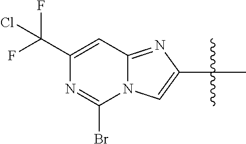 [SMILES: *c1cn2c(Br)nc(C(F)(F)Cl)cc2n1] | C-193 |
| 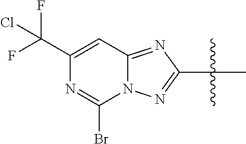 [SMILES: *c1nc2cc(C(F)(F)Cl)nc(Br)n2n1] | C-194 |
| 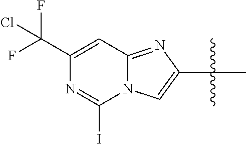 [SMILES: *c1cn2c(I)nc(C(F)(F)Cl)cc2n1] | C-195 |
| 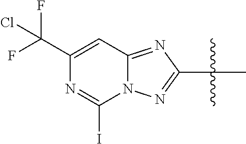 [SMILES: *c1nc2cc(C(F)(F)Cl)nc(I)n2n1] | C-196 |
| 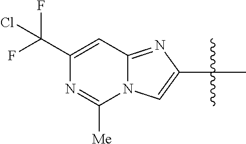 [SMILES: *c1cn2c(C)nc(C(F)(F)Cl)cc2n1] | C-197 |
| 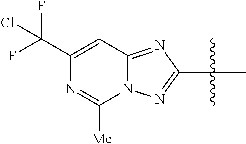 [SMILES: *c1nc2cc(C(F)(F)Cl)nc(C)n2n1] | C-198 |
| 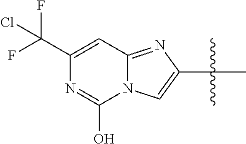 [SMILES: *c1cn2c(O)nc(C(F)(F)Cl)cc2n1] | C-199 |
| 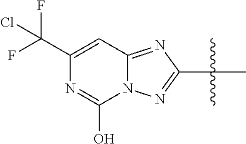 [SMILES: *c1nc2cc(C(F)(F)Cl)nc(O)n2n1] | C-200 |
| 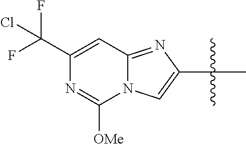 [SMILES: *c1cn2c(OC)nc(C(F)(F)Cl)cc2n1] | C-201 |
| 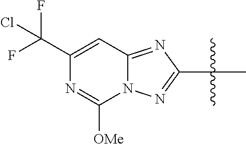 [SMILES: *c1nc2cc(C(F)(F)Cl)nc(OC)n2n1] | C-202 |
| 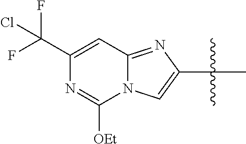 [SMILES: *c1cn2c(OCC)nc(C(F)(F)Cl)cc2n1] | C-203 |
| 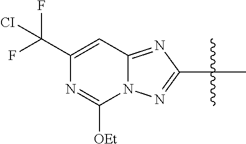 | C-204 |
| 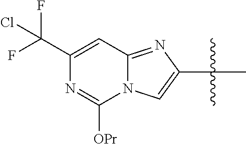 | C-205 |
| 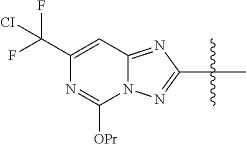 | C-206 |
| 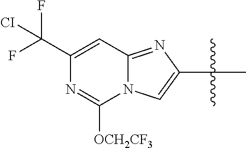 | C-207 |
| 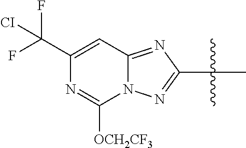 | C-208 |
| 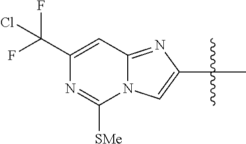 [SMILES: *c1cn2c(SC)nc(C(F)(F)Cl)cc2n1] | C-209 |
| 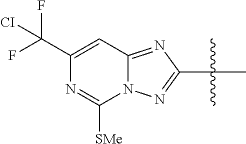 | C-210 |
| 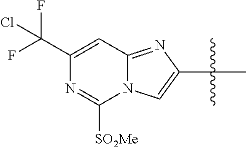 [SMILES: *c1cn2c(S(C)(=O)=O)nc(C(F)(F)Cl)cc2n1] | C-211 |
| 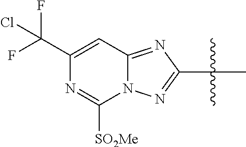 [SMILES: *c1nc2cc(C(F)(F)Cl)nc(S(C)(=O)=O)n2n1] | C-212 |
| 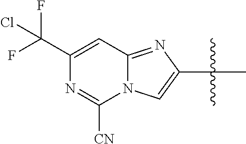 [SMILES: *c1cn2c(C#N)nc(C(F)(F)Cl)cc2n1] | C-213 |
| 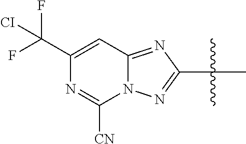 | C-214 |
| 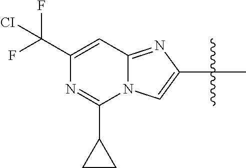 | C-215 |
| 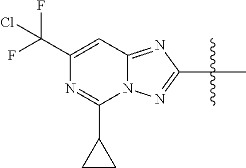 [SMILES: *c1nc2cc(C(F)(F)Cl)nc(C3CC3)n2n1] | C-216 |
| 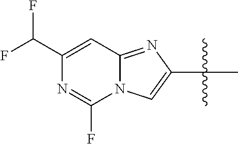 [SMILES: *c1cn2c(F)nc(C(F)F)cc2n1] | C-217 |
| 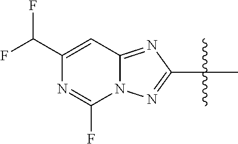 [SMILES: *c1nc2cc(C(F)F)nc(F)n2n1] | C-218 |
| 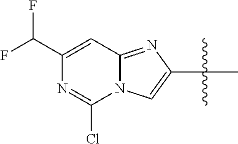 [SMILES: *c1cn2c(Cl)nc(C(F)F)cc2n1] | C-219 |
| 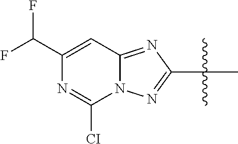 | C-220 |
| 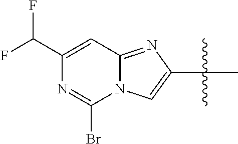 [SMILES: *c1cn2c(Br)nc(C(F)F)cc2n1] | C-221 |
| 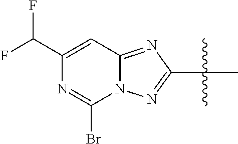 [SMILES: *c1nc2cc(C(F)F)nc(Br)n2n1] | C-222 |
| 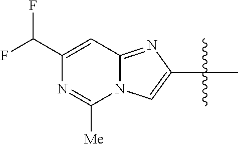 [SMILES: *c1cn2c(C)nc(C(F)F)cc2n1] | C-223 |
| 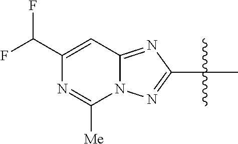 [SMILES: *c1nc2cc(C(F)F)nc(C)n2n1] | C-224 |

TABLE 2

| | |
|---|---|
| 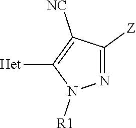 | Pyra-1 |
| 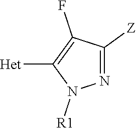 | Pyra-2 |
| 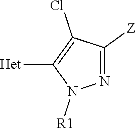 | Pyra-3 |
| 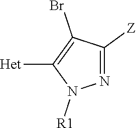 | Pyra-4 |
| 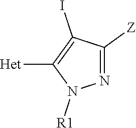 | Pyra-5 |
| 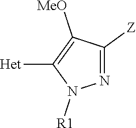 | Pyra-6 |
| 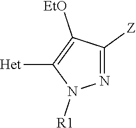 | Pyra-7 |
| 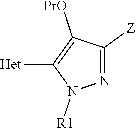 | Pyra-8 |
| 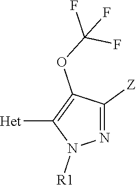 | Pyra-9 |
| 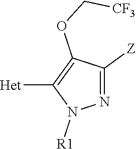 | Pyra-10 |
| 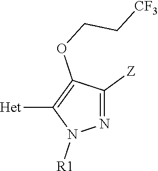 | Pyra-11 |
| 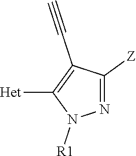 | Pyra-12 |
| 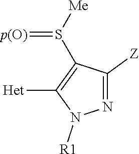 | Pyra-13 |
| 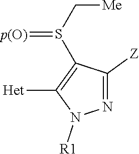 | Pyra-14 |
| 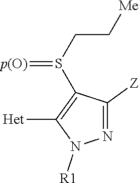 | Pyra-15 |
| 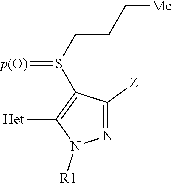 | Pyra-16 |
| 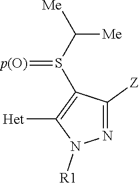 | Pyra-17 |
| 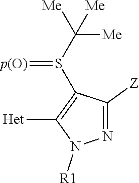 | Pyra-18 |
| 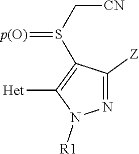 | Pyra-19 |
| 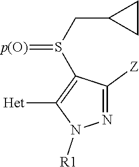 | Pyra-20 |
| 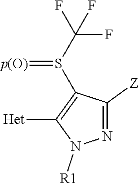 | Pyra-21 |
| 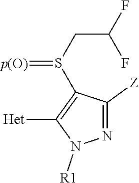 | Pyra-22 |
| 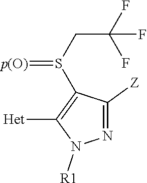 | Pyra-23 |
| 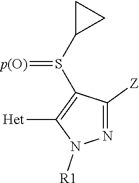 | Pyra-24 |
| 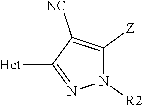 | Pyra-25 |
| 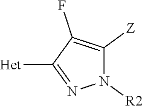 | Pyra-26 |
| 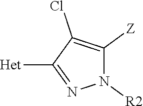 | Pyra-27 |
| 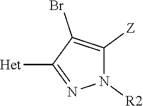 | Pyra-28 |
| 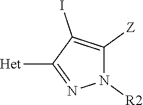 | Pyra-29 |
| 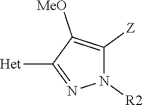 | Pyra-30 |
| 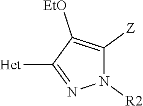 | Pyra-31 |
| 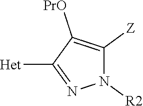 | Pyra-32 |
| 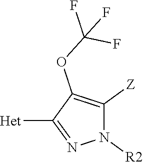 | Pyra-33 |
| 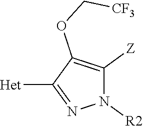 | Pyra-34 |
| 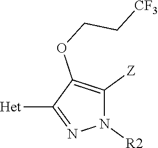 | Pyra-35 |
| 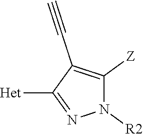 | Pyra-36 |
| 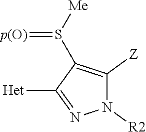 | Pyra-37 |
| 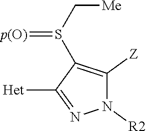 | Pyra-38 |
| 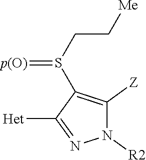 | Pyra-39 |
| 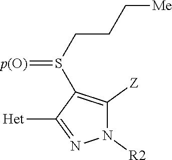 | Pyra-40 |
| 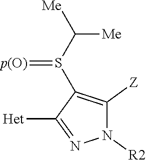 | Pyra-41 |
| 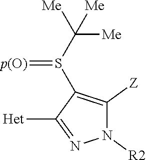 | Pyra-42 |
| 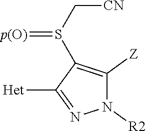 | Pyra-43 |
| 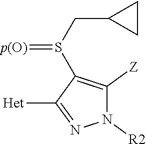 | Pyra-44 |
| 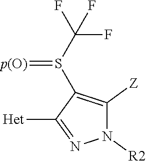 | Pyra-45 |
| 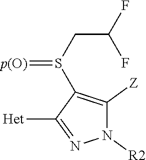 | Pyra-46 |
| 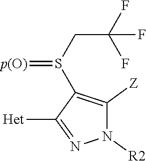 | Pyra-47 |
| 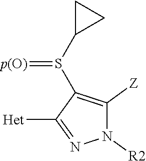 | Pyra-48 |

Hereinafter, for example, the description of “2-F-Ph” in Tables 3 and 4 means a phenyl group to which a fluorine atom is bonded at the 2-position, the description of “2-Me-Ph” means a phenyl group to which a methyl group is bonded at the 2-position, 2-Py represents a pyridin-2-yl group, 3-Py represents a pyridin-3-yl group, 4-Py represents a pyridin-4-yl group, 1-Pyra represents a 1H-pyrazol-1-yl group, 1-Tria represents a 1H-triazol-l-yl group, and other descriptions are the same.

TABLE 3

| No. | Substituent example of R1 | No. | Substituent example of R2 |
|---|---|---|---|
| R1-1 | —H | R2-1 | —H |
| R1-2 | —Me | R2-2 | —Me |
| R1-3 | —Et | R2-3 | —Et |
| R1-4 | —Pr | R2-4 | —Pr |
| R1-5 | —i-Pr | R2-5 | —i-Pr |
| R1-6 | —Bu | R2-6 | —Bu |
| R1-7 | —sec-Bu | R2-7 | —sec-Bu |
| R1-8 | —i-Bu | R2-8 | —i-Bu |
| R1-9 | —t-Bu | R2-9 | —t-Bu |
| R1-10 | —Pent | R2-10 | —Pent |
| R1-11 | —Hex | R2-11 | —Hex |
| R1-12 | —CH(CH₃)CH₂CH₂CH₃ | R2-12 | —CH(CH₃)CH₂CH₂CH₃ |
| R1-13 | —CH(CH₃)CH(CH₃)₂ | R2-13 | —CH(CH₃)CH(CH₃)₂ |
| R1-14 | —C(CH₃)₂CH₂CH₃ | R2-14 | —C(CH₃)₂CH₂CH₃ |
| R1-15 | —CH(CH₂CH₃)₂ | R2-15 | —CH(CH₂CH₃)₂ |
| R1-16 | —CH₂CH₂CH(CH₃)₂ | R2-16 | —CH₂CH₂CH(CH₃)₂ |
| R1-17 | —CH₂CH₂CH₂CH(CH₃)₂ | R2-17 | —CH₂CH₂CH₂CH(CH₃)₂ |
| R1-18 | —CH₂CH₂CH(CH₃)CH₂CH₃ | R2-18 | —CH₂CH₂CH(CH₃)CH₂CH₃ |
| R1-19 | —CH₂CH(CH₃)CH₂CH₂CH₃ | R2-19 | —CH₂CH(CH₃)CH₂CH₂CH₃ |
| R1-20 | —CH(CH₃)CH₂CH₂CH₂CH₃ | R2-20 | —CH(CH₃)CH₂CH₂CH₂CH₃ |
| R1-21 | —CH(CH₃)CH₂CH(CH₃)₂ | R2-21 | —CH(CH₃)CH₂CH(CH₃)₂ |
| R1-22 | —CH(CH₃)CH(CH₃)CH₂CH₃ | R2-22 | —CH(CH₃)CH(CH₃)CH₂CH₃ |
| R1-23 | —C(CH₃)₂CH₂CH₂CH₃ | R2-23 | —C(CH₃)₂CH₂CH₂CH₃ |
| R1-24 | —CH(CH₃)C(CH₃)₃ | R2-24 | —CH(CH₃)C(CH₃)₃ |
| R1-25 | —C(CH₃)₂CH(CH₃)₂ | R2-25 | —C(CH₃)₂CH(CH₃)₂ |
| R1-26 | —CH₂CH₂C(CH₃)₃ | R2-26 | —CH₂CH₂C(CH₃)₃ |
| R1-27 | —CH₂CH(CH₃)CH(CH₃)₂ | R2-27 | —CH₂CH(CH₃)CH(CH₃)₂ |
| R1-28 | —CH₂C(CH₃)₂CH₂CH₃ | R2-28 | —CH₂C(CH₃)₂CH₂CH₃ |
| R1-29 | —CFH₂ | R2-29 | —CFH₂ |
| R1-30 | —CF₂H | R2-30 | —CF₂H |
| R1-31 | —CF₃ | R2-31 | —CF₃ |
| R1-32 | —CH₂Cl | R2-32 | —CH₂Cl |
| R1-33 | —CHCl₂ | R2-33 | —CHCl₂ |
| R1-34 | —CCl₃ | R2-34 | —CCl₃ |
| R1-35 | —CH₂Br | R2-35 | —CH₂Br |
| R1-36 | —CHBr₂ | R2-36 | —CHBr₂ |
| R1-37 | —CBr₃ | R2-37 | —CBr₃ |
| R1-38 | —CH₂I | R2-38 | —CH₂I |
| R1-39 | —CHI₂ | R2-39 | —CHI₂ |
| R1-40 | —Cl₃ | R2-40 | —Cl₃ |
| R1-41 | —CH₂CF₂H | R2-41 | —CH₂CF₂H |
| R1-42 | —CH₂CF₃ | R2-42 | —CH₂CF₃ |
| R1-43 | —CH₂CH₂CF₂H | R2-43 | —CH₂CH₂CF₂H |
| R1-44 | —CH₂CH₂CF₃ | R2-44 | —CH₂CH₂CF₃ |
| R1-45 | —CH₂CH₂CH₂CF₂H | R2-45 | —CH₂CH₂CH₂CF₂H |
| R1-46 | —CH₂CH₂CH₂CF₃ | R2-46 | —CH₂CH₂CH₂CF₃ |
| R1-47 | —CF₂CF₂H | R2-47 | —CF₂CF₂H |
| R1-48 | —CF₂CF₃ | R2-48 | —CF₂CF₃ |
| R1-49 | —CFHCF₃ | R2-49 | —CFHCF₃ |
| R1-50 | —CH₂CF₂CF₂H | R2-50 | —CH₂CF₂CF₂H |
| R1-51 | —CH₂CF₂CF₃ | R2-51 | —CH₂CF₂CF₃ |
| R1-52 | —CF₂CF₂CF₃ | R2-52 | —CF₂CF₂CF₃ |
| R1-53 | —CH₂CF₂CF₂CF₃ | R2-53 | —CH₂CF₂CF₂CF₃ |
| R1-54 | —CF₂CF₂CF₂CF₃ | R2-54 | —CF₂CF₂CF₂CF₃ |
| R1-55 | —CH₂CF₂CF₂CF₂CF₃ | R2-55 | —CH₂CF₂CF₂CF₂CF₃ |
| R1-56 | c-Pr | R2-56 | c-Pr |
| R1-57 | c-Bu | R2-57 | c-Bu |
| R1-58 | c-Pent | R2-58 | c-Pent |
| R1-59 | c-Hex | R2-59 | c-Hex |
| R1-60 | c-Hept | R2-60 | c-Hept |
| R1-61 | c-Oct | R2-61 | c-Oct |
| R1-62 | —CH=CH₂ | R2-62 | —CH=CH₂ |
| R1-63 | —CH₂CH=CH₂ | R2-63 | —CH₂CH=CH₂ |
| R1-64 | —CH=CHCH₃ | R2-64 | —CH=CHCH₃ |
| R1-65 | —CH₂C(CH₃)=CH₂ | R2-65 | —CH₂C(CH₃)=CH₂ |
| R1-66 | —CH₂CH₂CH=CH₂ | R2-66 | —CH₂CH₂CH=CH₂ |
| R1-67 | —CH₂CH=CHCH₃ | R2-67 | —CH₂CH=CHCH₃ |
| R1-68 | —CH=CHCH₂CH₃ | R2-68 | —CH=CHCH₂CH₃ |
| R1-69 | —CH₂CH=C(CH₃)₂ | R2-69 | —CH₂CH=C(CH₃)₂ |
| R1-70 | —CH₂CH₂CH=C(CH₃)₂ | R2-70 | —CH₂CH₂CH=C(CH₃)₂ |
| R1-71 | —CH=CFH | R2-71 | —CH=CFH |
| R1-72 | —CH=CF₂ | R2-72 | —CH=CF₂ |
| R1-73 | —CH=CCl₂ | R2-73 | —CH=CCl₂ |
| R1-74 | —CH₂CH=CFH | R2-74 | —CH₂CH=CFH |
| R1-75 | —CH₂CH=CF₂ | R2-75 | —CH₂CH=CF₂ |
| R1-76 | —CH₂CH=CCl₂ | R2-76 | —CH₂CH=CCl₂ |
| R1-77 | —CH₂CH₂CH=CF₂ | R2-77 | —CH₂CH₂CH=CF₂ |
| R1-78 | —CH₂CH₂CH₂CH=CF₂ | R2-78 | —CH₂CH₂CH₂CH=CF₂ |
| R1-79 | —CH₂CH₂CH₂CH₂CH=CF₂ | R2-79 | —CH₂CH₂CH₂CH₂CH=CF₂ |
| R1-80 | —C≡CH | R2-80 | —C≡CH |
| R1-81 | —C≡CCH₃ | R2-81 | —C≡CCH₃ |
| R1-82 | —CH₂C≡CH | R2-82 | —CH₂C≡CH |
| R1-83 | —C≡CCH₂CH₃ | R2-83 | —C≡CCH₂CH₃ |
| R1-84 | —CH₂C≡CCH₃ | R2-84 | —CH₂C≡CCH₃ |
| R1-85 | —CH₂CH₂C≡CH | R2-85 | —CH₂CH₂C≡CH |
| R1-86 | —C≡CCH₂CH₂CH₃ | R2-86 | —C≡CCH₂CH₂CH₃ |
| R1-87 | —CH₂C≡CCH₂CH₃ | R2-87 | —CH₂C≡CCH₂CH₃ |
| R1-88 | —C(CH₃)₂C≡CH | R2-88 | —C(CH₃)₂C≡CH |
| R1-89 | —C≡CF | R2-89 | —C≡CF |
| R1-90 | —C≡CCF₂H | R2-90 | —C≡CCF₂H |
| R1-91 | —C≡CCF₃ | R2-91 | —C≡CCF₃ |
| R1-92 | —C≡CCH₂CF₂H | R2-92 | —C≡CCH₂CF₂H |
| R1-93 | —C≡CCH₂CF₃ | R2-93 | —C≡CCH₂CF₃ |
| R1-94 | —CH₂C≡CHCF₂H | R2-94 | —CH₂C≡CHCF₂H |
| R1-95 | —CH₂C≡CCF₃ | R2-95 | —CH₂C≡CCF₃ |
| R1-96 | —Ph | R2-96 | —Ph |
| R1-97 | —2-F—Ph | R2-97 | —2-F—Ph |
| R1-98 | —3-F—Ph | R2-98 | —3-F—Ph |
| R1-99 | —4-F—Ph | R2-99 | —4-F—Ph |
| R1-100 | —2-Cl—Ph | R2-100 | —2-Cl—Ph |
| R1-101 | —3-Cl—Ph | R2-101 | —3-Cl—Ph |
| R1-102 | —4-Cl—Ph | R2-102 | —4-Cl—Ph |
| R1-103 | —2-Br—Ph | R2-103 | —2-Br—Ph |
| R1-104 | —3-Br—Ph | R2-104 | —3-Br—Ph |
| R1-105 | —4-Br—Ph | R2-105 | —4-Br—Ph |
| R1-106 | —2-I—Ph | R2-106 | —2-I—Ph |
| R1-107 | —3-I—Ph | R2-107 | —3-I—Ph |
| R1-108 | —4-I—Ph | R2-108 | —4-I—Ph |
| R1-109 | —2-N≡C—Ph | R2-109 | —2-N≡C—Ph |
| R1-110 | —3-N≡C—Ph | R2-110 | —3-N≡C—Ph |
| R1-111 | —4-N≡C—Ph | R2-111 | —4-N≡C—Ph |
| R1-112 | —2-Me—Ph | R2-112 | —2-Me—Ph |
| R1-113 | —3-Me—Ph | R2-113 | —3-Me—Ph |
| R1-114 | —4-Me—Ph | R2-114 | —4-Me—Ph |
| R1-115 | —2-Et—Ph | R2-115 | —2-Et—Ph |
| R1-116 | —3-Et—Ph | R2-116 | —3-Et—Ph |
| R1-117 | —4-Et—Ph | R2-117 | —4-Et—Ph |
| R1-118 | —2-i-Pr—Ph | R2-118 | —2-i-Pr—Ph |
| R1-119 | —3-i-Pr—Ph | R2-119 | —3-i-Pr—Ph |
| R1-120 | —4-i-Pr—Ph | R2-120 | —4-i-Pr—Ph |
| R1-121 | —2-CF₂H—Ph | R2-121 | —2-CF₂H—Ph |
| R1-122 | —3-CF₂H—Ph | R2-122 | —3-CF₂H—Ph |
| R1-123 | —4-CF₂H—Ph | R2-123 | —4-CF₂H—Ph |
| R1-124 | —2-CF₃—Ph | R2-124 | —2-CF₃—Ph |
| R1-125 | —3-CF₃—Ph | R2-125 | —3-CF₃—Ph |
| R1-126 | —4-CF₃—Ph | R2-126 | —4-CF₃—Ph |
| R1-127 | —2-c-Pr—Ph | R2-127 | —2-c-Pr—Ph |
| R1-128 | —3-c-Pr—Ph | R2-128 | —3-c-Pr—Ph |
| R1-129 | —4-c-Pr—Ph | R2-129 | —4-c-Pr—Ph |
| R1-130 | —2-OMe—Ph | R2-130 | —2-OMe—Ph |
| R1-131 | —3-OMe—Ph | R2-131 | —3-OMe—Ph |
| R1-132 | —4-OMe—Ph | R2-132 | —4-OMe—Ph |
| R1-133 | —2-OEt—Ph | R2-133 | —2-OEt—Ph |
| R1-134 | —3-OEt—Ph | R2-134 | —3-OEt—Ph |
| R1-135 | —4-OEt—Ph | R2-135 | —4-OEt—Ph |
| R1-136 | —2-OCF₂H—Ph | R2-136 | —2-OCF₂H—Ph |
| R1-137 | —3-OCF₂H—Ph | R2-137 | —3-OCF₂H—Ph |
| R1-138 | —4-OCF₂H—Ph | R2-138 | —4-OCF₂H—Ph |
| R1-139 | —2-OCF₃—Ph | R2-139 | —2-OCF₃—Ph |
| R1-140 | —3-OCF₃—Ph | R2-140 | —3-OCF₃—Ph |
| R1-141 | —4-OCF₃—Ph | R2-141 | —4-OCF₃—Ph |
| R1-142 | —2-SMe—Ph | R2-142 | —2-SMe—Ph |
| R1-143 | —3-SMe—Ph | R2-143 | —3-SMe—Ph |
| R1-144 | —4-SMe—Ph | R2-144 | —4-SMe—Ph |
| R1-145 | —2-S(═O)Me—Ph | R2-145 | —2-S(═O)Me—Ph |
| R1-146 | —3-S(═O)Me—Ph | R2-146 | —3-S(═O)Me—Ph |
| R1-147 | —4-S(═O)Me—Ph | R2-147 | —4-S(═O)Me—Ph |
| R1-148 | —2-SO₂Me—Ph | R2-148 | —2-SO₂Me—Ph |
| R1-149 | —3-SO₂Me—Ph | R2-149 | —3-SO₂Me—Ph |
| R1-150 | —4-SO₂Me—Ph | R2-150 | —4-SO₂Me—Ph |
| R1-151 | —2-SCF₃—Ph | R2-151 | —2-SCF₃—Ph |
| R1-152 | —3-SCF₃—Ph | R2-152 | —3-SCF₃—Ph |
| R1-153 | —4-SCF₃—Ph | R2-153 | —4-SCF₃—Ph |
| R1-154 | —2-S(═O)CF₃—Ph | R2-154 | —2-S(═O)CF₃—Ph |
| R1-155 | —3-S(═O)CF₃—Ph | R2-155 | —3-S(═O)CF₃—Ph |
| R1-156 | —4-S(═O)CF₃—Ph | R2-156 | —4-S(═O)CF₃—Ph |
| R1-157 | —2-SO₂CF₃—Ph | R2-157 | —2-SO₂CF₃—Ph |
| R1-158 | —3-SO₂CF₃—Ph | R2-158 | —3-SO₂CF₃—Ph |
| R1-159 | —4-SO₂CF₃—Ph | R2-159 | —4-SO₂CF₃—Ph |
| R1-160 | —C(═O)H | R2-160 | —C(═O)H |
| R1-161 | —C(═O)Me | R2-161 | —C(═O)Me |
| R1-162 | —C(═O)CH₂OMe | R2-162 | —C(═O)CH₂OMe |
| R1-163 | —C(═O)CH₂C≡N | R2-163 | —C(═O)CH₂C≡N |
| R1-164 | —C(═O)Et | R2-164 | —C(═O)Et |
| R1-165 | —C(═O)CF₂H | R2-165 | —C(═O)CF₂H |
| R1-166 | —C(═O)CF₃ | R2-166 | —C(═O)CF₃ |
| R1-167 | —C(═O)c-Pr | R2-167 | —C(═O)c-Pr |
| R1-168 | —C(═O)Ph | R2-168 | —C(═O)Ph |
| R1-169 | —C(═O)-4-F—Ph | R2-169 | —C(═O)-4-F—Ph |
| R1-170 | —C(═O)-4-CF₃—Ph | R2-170 | —C(═O)-4-CF₃—Ph |
| R1-171 | —C(═O)OMe | R2-171 | —C(═O)OMe |
| R1-172 | —C(═O)OEt | R2-172 | —C(═O)OEt |
| R1-173 | —C(═O)OPr | R2-173 | —C(═O)OPr |
| R1-174 | —C(═O)O-i-Pr | R2-174 | —C(═O)O-i-Pr |
| R1-175 | —C(═O)O-c-Pr | R2-175 | —C(═O)O-c-Pr |
| R1-176 | —C(═O)OCH₂C≡CH | R2-176 | —C(═O)OCH₂C≡CH |
| R1-177 | —C(═O)OPh | R2-177 | —C(═O)OPh |
| R1-178 | —C(═O)O-4-NO₂—Ph | R2-178 | —C(═O)O-4-NO₂—Ph |
| R1-179 | —SO₂Me | R2-179 | —SO₂Me |
| R1-180 | —SO₂Et | R2-180 | —SO₂Et |
| R1-181 | —SO₂CF₃ | R2-181 | —SO₂CF₃ |
| R1-182 | —SO₂CF₂H | R2-182 | —SO₂CF₂H |
| R1-183 | —SO₂CH₂CF₃ | R2-183 | —SO₂CH₂CF₃ |
| R1-184 | —SO₂CH₂-c-Pr | R2-184 | —SO₂CH₂-c-Pr |
| R1-185 | —SO₂Ph | R2-185 | —SO₂Ph |
| R1-186 | —SO₂(4-F—Ph) | R2-186 | —SO₂(4-F—Ph) |
| R1-187 | —SO₂(4-Cl—Ph) | R2-187 | —SO₂(4-Cl—Ph) |
| R1-188 | —SO₂(4-Me—Ph) | R2-188 | —SO₂(4-Me—Ph) |
| R1-189 | —SO₂(4-CF₃—Ph) | R2-189 | —SO₂(4-CF₃—Ph) |
| R1-190 | —CH₂C≡N | R2-190 | —CH₂C≡N |
| R1-191 | —C(Me)C≡N | R2-191 | —C(Me)C≡N |
| R1-192 | —CH₂CH₂C≡N | R2-192 | —CH₂CH₂C≡N |
| R1-193 | —CH₂CH₂CH₂C≡N | R2-193 | —CH₂CH₂CH₂C≡N |
| R1-194 | —CH₂-c-Pr | R2-194 | —CH₂-c-Pr |
| R1-195 | —CH₂OH | R2-195 | —CH₂OH |
| R1-196 | —CH₂-c-Bu | R2-196 | —CH₂-c-Bu |
| R1-197 | —CH₂OMe | R2-197 | —CH₂OMe |
| R1-198 | —CH₂OEt | R2-198 | —CH₂OEt |
| R1-199 | —CH₂OPr | R2-199 | —CH₂OPr |
| R1-200 | —CH₂O-i-Pr | R2-200 | —CH₂O-i-Pr |
| R1-201 | —CH₂O-c-Pr | R2-201 | —CH₂O-c-Pr |
| R1-202 | —CH₂CH₂OMe | R2-202 | —CH₂CH₂OMe |
| R1-203 | —CH₂CH₂OEt | R2-203 | —CH₂CH₂OEt |

Substituent examples of R1 and R2

| | | | | | |
|---|---|---|---|---|---|
| R1-, R2-500 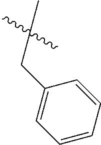 [SMILES: *Cc1ccccc1] | R1-, R2-501 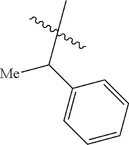 [SMILES: *C(C)c1ccccc1] | R1-, R2-502 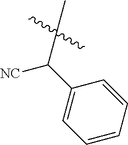 [SMILES: *C(C#N)c1ccccc1] | R1-, R2-503 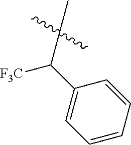 [SMILES: *C(c1ccccc1)C(F)(F)F] | R1-, R2-504 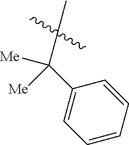 [SMILES: *C(C)(C)c1ccccc1] | R1-, R2-505 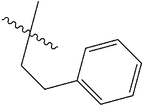 [SMILES: *CCc1ccccc1] |
| R1, R2-506 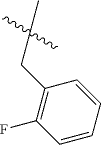 [SMILES: *Cc1ccccc1F] | R1, R2-507 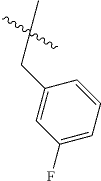 [SMILES: *Cc1cccc(F)c1] | R1, R2-508 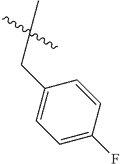 [SMILES: *Cc1ccc(F)cc1] | R1, R2-509 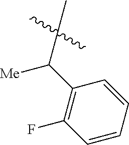 [SMILES: *C(C)c1ccccc1F] | R1, R2-510 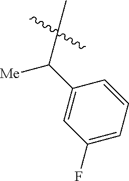 [SMILES: *C(C)c1cccc(F)c1] | R1, R2-511 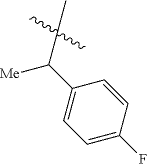 [SMILES: *C(C)c1ccc(F)cc1] |
| R1-, R2-512 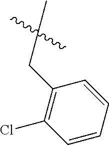 [SMILES: *Cc1ccccc1Cl] | R1-, R2-513 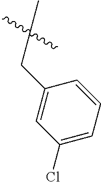 [SMILES: *Cc1cccc(Cl)c1] | R1-, R2-514 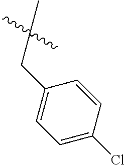 [SMILES: *Cc1ccc(Cl)cc1] | R1-, R2-515 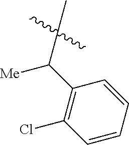 [SMILES: *C(C)c1ccccc1Cl] | R1-, R2-516 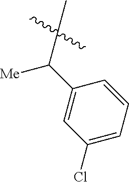 [SMILES: *C(C)c1cccc(Cl)c1] | R1-, R2-517 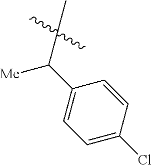 [SMILES: *C(C)c1ccc(Cl)cc1] |
| R1, R2-518 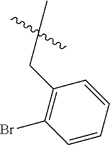 [SMILES: *Cc1ccccc1Br] | R1, R2-519 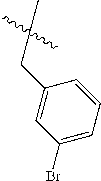 [SMILES: *Cc1cccc(Br)c1] | R1, R2-520 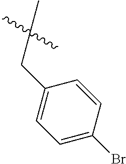 [SMILES: *Cc1ccc(Br)cc1] | R1, R2-521 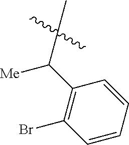 [SMILES: *C(C)c1ccccc1Br] | R1, R2-522 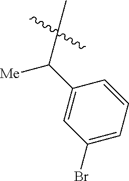 [SMILES: *C(C)c1cccc(Br)c1] | R1, R2-523 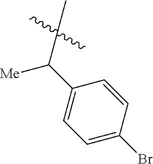 [SMILES: *C(C)c1ccc(Br)cc1] |
| R1-, R2-524 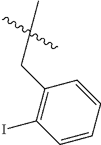 [SMILES: *Cc1ccccc1I] | R1-, R2-525 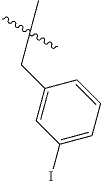 [SMILES: *Cc1cccc(I)c1] | R1-, R2-526 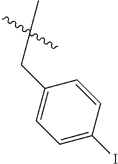 [SMILES: *Cc1ccc(I)cc1] | R1-, R2-527 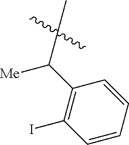 [SMILES: *C(C)c1ccccc1I] | R1-, R2-528 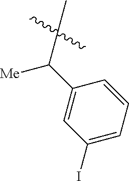 [SMILES: *C(C)c1cccc(I)c1] | R1-, R2-529 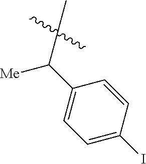 [SMILES: *C(C)c1ccc(I)cc1] |
| R1-, R2-530 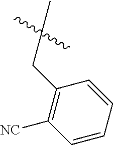 [SMILES: *Cc1ccccc1C#N] | R1-, R2-531 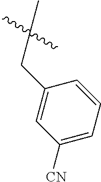 [SMILES: *Cc1cccc(C#N)c1] | R1-, R2-532 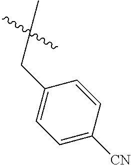 [SMILES: *Cc1ccc(C#N)cc1] | R1-, R2-533 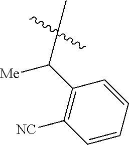 [SMILES: *C(C)c1ccccc1C#N] | R1-, R2-534 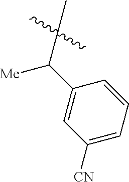 [SMILES: *C(C)c1cccc(C#N)c1] | R1-, R2-535 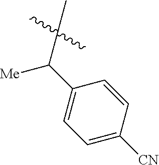 [SMILES: *C(C)c1ccc(C#N)cc1] |
| R1-, R2-536 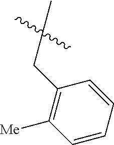 [SMILES: *Cc1ccccc1C] | R1-, R2-537 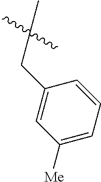 [SMILES: *Cc1cccc(C)c1] | R1-, R2-538 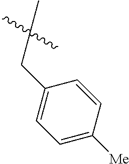 [SMILES: *Cc1ccc(C)cc1] | R1-, R2-539 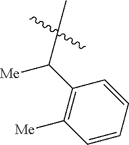 [SMILES: *C(C)c1ccccc1C] | R1-, R2-540 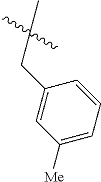 [SMILES: *Cc1cccc(C)c1] | R1-, R2-541 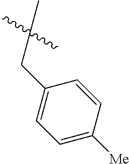 [SMILES: *Cc1ccc(C)cc1] |
| R1-, R2-542 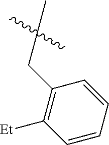 [SMILES: *Cc1ccccc1CC] | R1-, R2-543 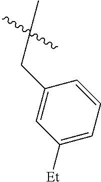 [SMILES: *Cc1cccc(CC)c1] | R1-, R2-544 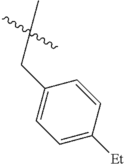 [SMILES: *Cc1ccc(CC)cc1] | R1-, R2-545 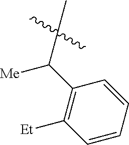 [SMILES: *C(C)c1ccccc1CC] | R1-, R2-546 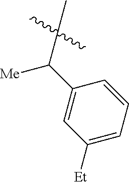 [SMILES: *C(C)c1cccc(CC)c1] | R1-, R2-547 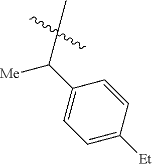 [SMILES: *C(C)c1ccc(CC)cc1] |
| R1-, R2-548 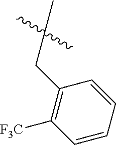 [SMILES: *Cc1ccccc1C(F)(F)F] | R1-, R2-549 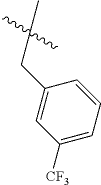 [SMILES: *Cc1cccc(C(F)(F)F)c1] | R1-, R2-550 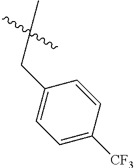 [SMILES: *Cc1ccc(C(F)(F)F)cc1] | R1-, R2-551 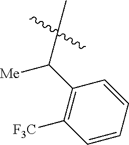 [SMILES: *C(C)c1ccccc1C(F)(F)F] | R1-, R2-552 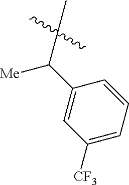 [SMILES: *C(C)c1cccc(C(F)(F)F)c1] | R1-, R2-553 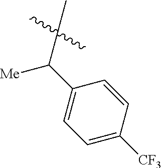 [SMILES: *C(C)c1ccc(C(F)(F)F)cc1] |
| R1-, R2-554 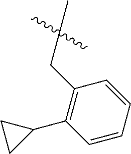 [SMILES: *Cc1ccccc1C1CC1] | R1-, R2-555 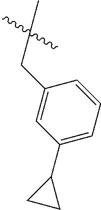 [SMILES: *Cc1cccc(C2CC2)c1] | R1-, R2-556 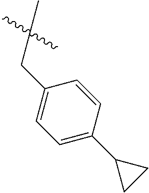 [SMILES: *Cc1ccc(C2CC2)cc1] | R1-, R2-557 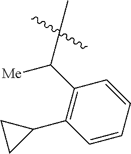 [SMILES: *C(C)c1ccccc1C1CC1] | R1-, R2-558 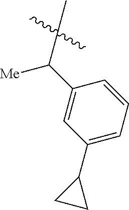 [SMILES: *C(C)c1cccc(C2CC2)c1] | R1-, R2-559 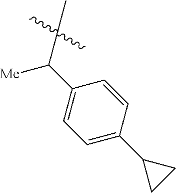 [SMILES: *C(C)c1ccc(C2CC2)cc1] |
| R1-, R2-560 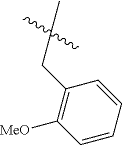 [SMILES: *Cc1ccccc1OC] | R1-, R2-561 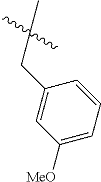 [SMILES: *Cc1cccc(OC)c1] | R1-, R2-562 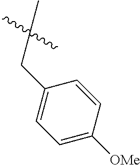 [SMILES: *Cc1ccc(OC)cc1] | R1-, R2-563 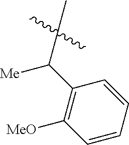 [SMILES: *C(C)c1ccccc1OC] | R1-, R2-564 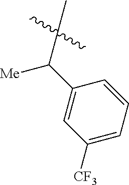 [SMILES: *C(C)c1cccc(C(F)(F)F)c1] | R1-, R2-565 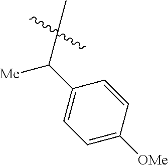 [SMILES: *C(C)c1ccc(OC)cc1] |
| R1-, R2-566 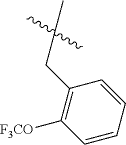 [SMILES: *Cc1ccccc1OC(F)(F)F] | R1-, R2-567 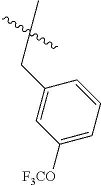 [SMILES: *Cc1cccc(OC(F)(F)F)c1] | R1-, R2-568 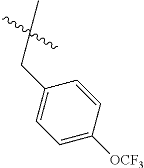 [SMILES: *Cc1ccc(OC(F)(F)F)cc1] | R1-, R2-569 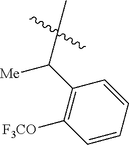 [SMILES: *C(C)c1ccccc1OC(F)(F)F] | R1-, R2-570 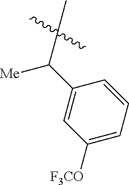 [SMILES: *C(C)c1cccc(OC(F)(F)F)c1] | R1-, R2-571 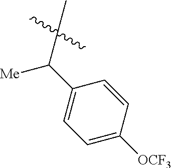 [SMILES: *C(C)c1ccc(OC(F)(F)F)cc1] |
| R1-, R2-572 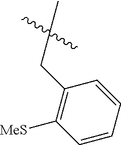 [SMILES: *Cc1ccccc1SC] | R1-, R2-573 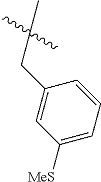 [SMILES: *Cc1cccc(SC)c1] | R1-, R2-574 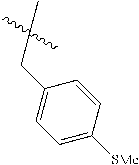 [SMILES: *Cc1ccc(SC)cc1] | R1-, R2-575 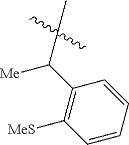 [SMILES: *C(C)c1ccccc1SC] | R1-, R2-576 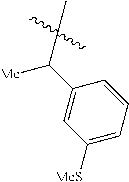 [SMILES: *C(C)c1cccc(SC)c1] | R1-, R2-577 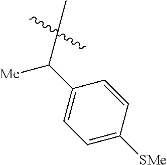 [SMILES: *C(C)c1ccc(SC)cc1] |
| R1-, R2-578 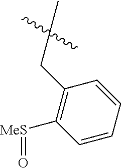 | R1-, R2-579 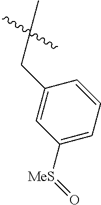 | R1-, R2-580 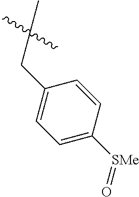 | R1-, R2-581 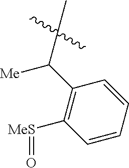 | R1-, R2-582 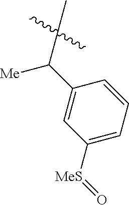 | R1-, R2-583 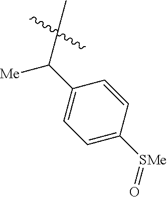 |
| R1-, R2-584 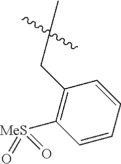 | R1-, R2-585 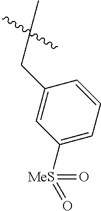 | R1-, R2-586 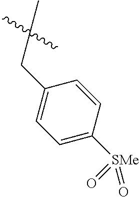 | R1-, R2-587 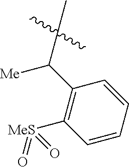 | R1-, R2-588 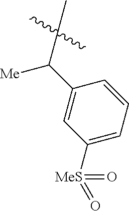 | R1-, R2-589 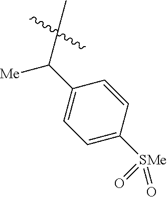 |
| R1-, R2-590 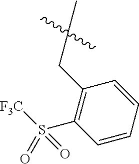 [SMILES: *Cc1ccccc1S(=O)(=O)C(F)(F)F] | R1-, R2-591 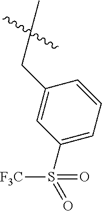 [SMILES: *Cc1cccc(S(=O)(=O)C(F)(F)F)c1] | R1-, R2-592 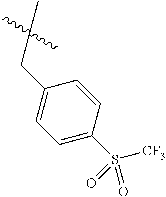 [SMILES: *Cc1ccc(S(=O)(=O)C(F)(F)F)cc1] | R1-, R2-593 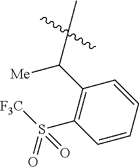 [SMILES: *C(C)c1ccccc1S(=O)(=O)C(F)(F)F] | R1-, R2-594 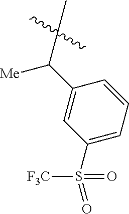 [SMILES: *C(C)c1cccc(S(=O)(=O)C(F)(F)F)c1] | R1-, R2-595 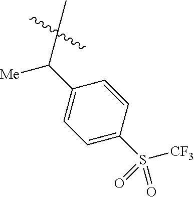 [SMILES: *C(C)c1ccc(S(=O)(=O)C(F)(F)F)cc1] |
| R1-, R2-596 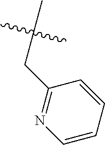 [SMILES: *Cc1ccccn1] | R1-, R2-597 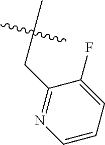 [SMILES: *Cc1ncccc1F] | R1-, R2-598 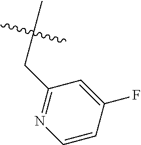 [SMILES: *Cc1cc(F)ccn1] | R1-, R2-599 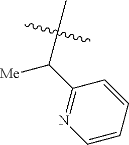 [SMILES: *C(C)c1ccccn1] | R1-, R2-600 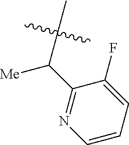 [SMILES: *C(C)c1ncccc1F] | R1-, R2-601 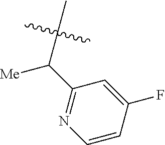 [SMILES: *C(C)c1cc(F)ccn1] |
| R1-, R2-602 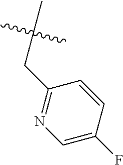 [SMILES: *Cc1ccc(F)cn1] | R1-, R2-603 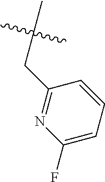 [SMILES: *Cc1cccc(F)n1] | R1-, R2-604 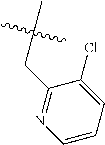 [SMILES: *Cc1ncccc1Cl] | R1-, R2-605 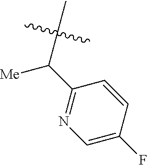 [SMILES: *C(C)c1ccc(F)cn1] | R1-, R2-606 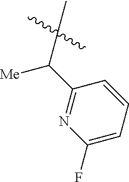 [SMILES: *C(C)c1cccc(F)n1] | R1-, R2-607 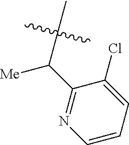 [SMILES: *C(C)c1ncccc1Cl] |
| R1-, R2-608 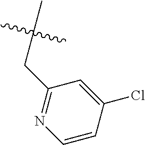 [SMILES: *Cc1cc(Cl)ccn1] | R1-, R2-609 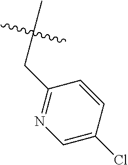 [SMILES: *Cc1ccc(Cl)cn1] | R1-, R2-610 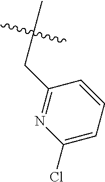 [SMILES: *Cc1cccc(Cl)n1] | R1-, R2-611 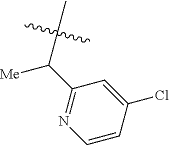 [SMILES: *C(C)c1cc(Cl)ccn1] | R1-, R2-612 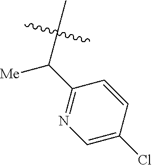 [SMILES: *C(C)c1ccc(Cl)cn1] | R1-, R2-613 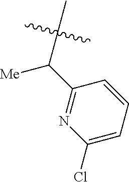 [SMILES: *C(C)c1cccc(Cl)n1] |
| R1-, R2-614 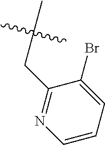 [SMILES: *Cc1ncccc1Br] | R1-, R2-615 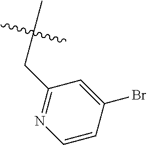 [SMILES: *Cc1cc(Br)ccn1] | R1-, R2-616 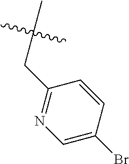 [SMILES: *Cc1ccc(Br)cn1] | R1-, R2-617 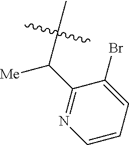 [SMILES: *C(C)c1ncccc1Br] | R1-, R2-618 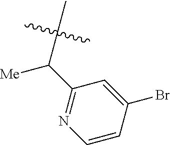 [SMILES: *C(C)c1cc(Br)ccn1] | R1-, R2-619 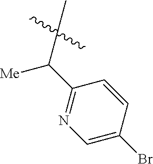 [SMILES: *C(C)c1ccc(Br)cn1] |
| R1-, R2-620 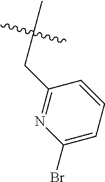 [SMILES: *Cc1cccc(Br)n1] | R1-, R2-621 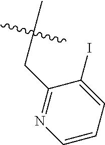 [SMILES: *Cc1ncccc1I] | R1-, R2-622 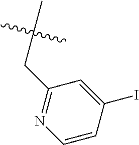 [SMILES: *Cc1cc(I)ccn1] | R1-, R2-623 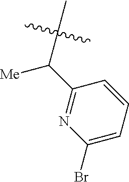 [SMILES: *C(C)c1cccc(Br)n1] | R1-, R2-624 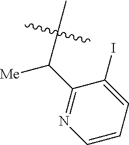 [SMILES: *C(C)c1ncccc1I] | R1-, R2-625 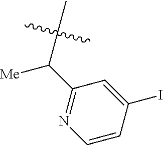 [SMILES: *C(C)c1cc(I)ccn1] |
| R1-, R2-626 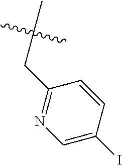 [SMILES: *Cc1ccc(I)cn1] | R1-, R2-627 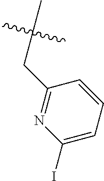 [SMILES: *Cc1cccc(I)n1] | R1-, R2-628 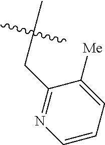 [SMILES: *Cc1ncccc1C] | R1-, R2-629 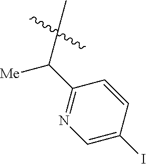 [SMILES: *C(C)c1ccc(I)cn1] | R1-, R2-630 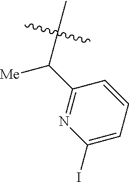 [SMILES: *C(C)c1cccc(I)n1] | R1-, R2-631 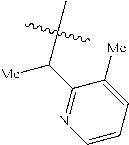 [SMILES: *C(C)c1ncccc1C] |
| R1-, R2-632 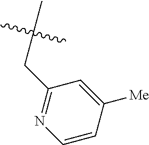 [SMILES: *Cc1cc(C)ccn1] | R1-, R2-633 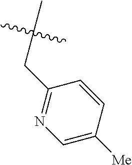 [SMILES: *Cc1ccc(C)cn1] | R1-, R2-634 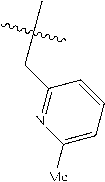 [SMILES: *Cc1cccc(C)n1] | R1-, R2-635 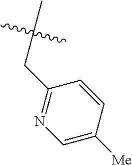 [SMILES: *Cc1ccc(C)cn1] | R1-, R2-636 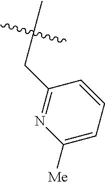 [SMILES: *Cc1cccc(C)n1] | R1-, R2-637 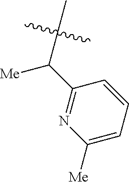 [SMILES: *C(C)c1cccc(C)n1] |
| R1-, R2-638 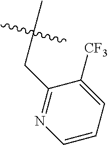 [SMILES: *Cc1ncccc1C(F)(F)F] | R1-, R2-639 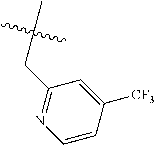 [SMILES: *Cc1cc(C(F)(F)F)ccn1] | R1-, R2-640 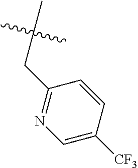 [SMILES: *Cc1ccc(C(F)(F)F)cn1] | R1-, R2-641 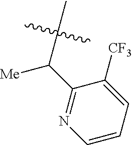 [SMILES: *C(C)c1ncccc1C(F)(F)F] | R1-, R2-642 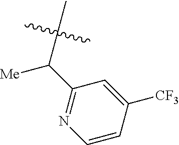 [SMILES: *C(C)c1cc(C(F)(F)F)ccn1] | R1-, R2-643 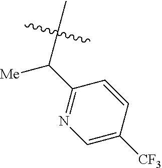 [SMILES: *C(C)c1ccc(C(F)(F)F)cn1] |
| R1-, R2-644 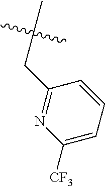 [SMILES: *Cc1cccc(C(F)(F)F)n1] | R1-, R2-645 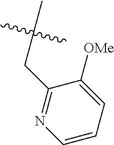 [SMILES: *Cc1ncccc1OC] | R1-, R2-646 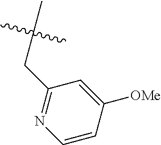 [SMILES: *Cc1cc(OC)ccn1] | R1-, R2-647 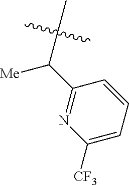 [SMILES: *C(C)c1cccc(C(F)(F)F)n1] | R1-, R2-648 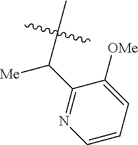 [SMILES: *C(C)c1ncccc1OC] | R1-, R2-649 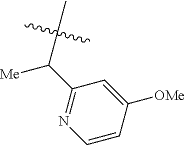 [SMILES: *C(C)c1cc(OC)ccn1] |
| R1-, R2-650 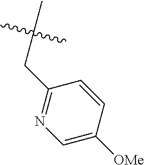 [SMILES: *Cc1ccc(OC)cn1] | R1-, R2-651 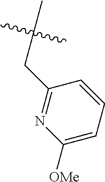 [SMILES: *Cc1cccc(OC)n1] | R1-, R2-652 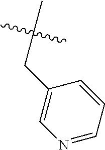 [SMILES: *Cc1cccnc1] | R1-, R2-653 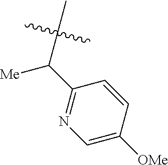 [SMILES: *C(C)c1ccc(OC)cn1] | R1-, R2-654 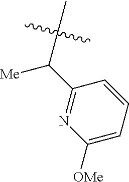 [SMILES: *C(C)c1cccc(OC)n1] | R1-, R2-655 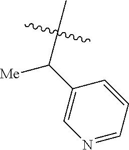 [SMILES: *C(C)c1cccnc1] |
| R1-, R2-656 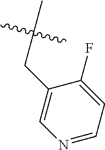 [SMILES: *Cc1cnccc1F] | R1-, R2-657 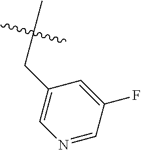 [SMILES: *Cc1cncc(F)c1] | R1-, R2-658 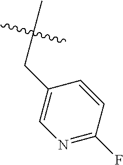 [SMILES: *Cc1ccc(F)nc1] | R1-, R2-659 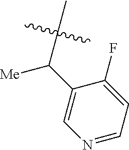 [SMILES: *C(C)c1cnccc1F] | R1-, R2-660 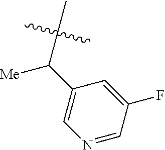 [SMILES: *C(C)c1cncc(F)c1] | R1-, R2-661 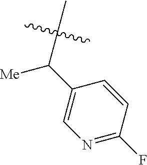 [SMILES: *C(C)c1ccc(F)nc1] |
| R1-, R2-662 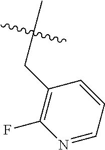 [SMILES: *Cc1cccnc1F] | R1-, R2-663 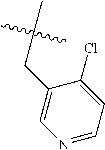 [SMILES: *Cc1cnccc1Cl] | R1-, R2-664 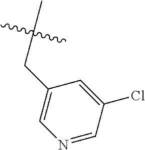 [SMILES: *Cc1cncc(Cl)c1] | R1-, R2-665 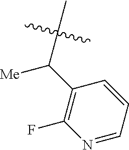 [SMILES: *C(C)c1cccnc1F] | R1-, R2-666 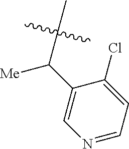 [SMILES: *C(C)c1cnccc1Cl] | R1-, R2-667 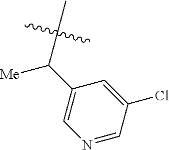 [SMILES: *C(C)c1cncc(Cl)c1] |
| R1-, R2-668 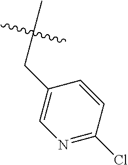 [SMILES: *Cc1ccc(Cl)nc1] | R1-, R2-669 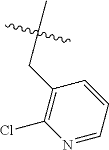 [SMILES: *Cc1cccnc1Cl] | R1-, R2-670 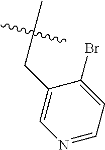 [SMILES: *Cc1cnccc1Br] | R1-, R2-671 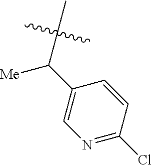 [SMILES: *C(C)c1ccc(Cl)nc1] | R1-, R2-672 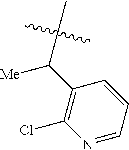 [SMILES: *C(C)c1cccnc1Cl] | R1-, R2-673 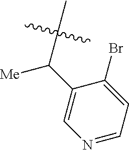 [SMILES: *C(C)c1cnccc1Br] |
| R1-, R2-674 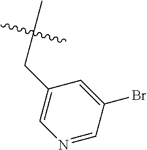 [SMILES: *Cc1cncc(Br)c1] | R1-, R2-675 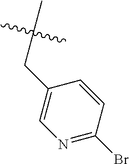 [SMILES: *Cc1ccc(Br)nc1] | R1-, R2-676 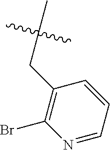 [SMILES: *Cc1cccnc1Br] | R1-, R2-677 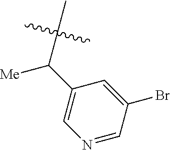 [SMILES: *C(C)c1cncc(Br)c1] | R1-, R2-678 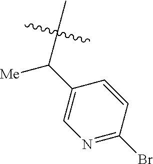 [SMILES: *C(C)c1ccc(Br)nc1] | R1-, R2-679 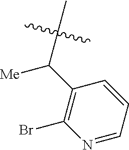 [SMILES: *C(C)c1cccnc1Br] |
| R1-, R2-680 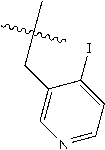 [SMILES: *Cc1cnccc1I] | R1-, R2-681 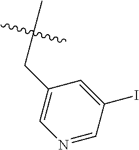 [SMILES: *Cc1cncc(I)c1] | R1-, R2-682 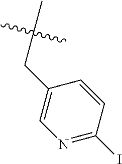 [SMILES: *Cc1ccc(I)nc1] | R1-, R2-683 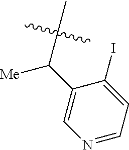 [SMILES: *C(C)c1cnccc1I] | R1-, R2-684 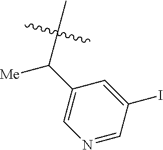 [SMILES: *C(C)c1cncc(I)c1] | R1-, R2-685 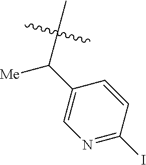 [SMILES: *C(C)c1ccc(I)nc1] |
| R1-, R2-686 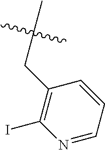 [SMILES: *Cc1cccnc1I] | R1-, R2-687 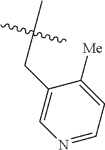 [SMILES: *Cc1cnccc1C] | R1-, R2-688 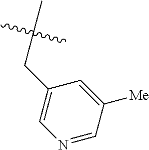 [SMILES: *Cc1cncc(C)c1] | R1-, R2-689 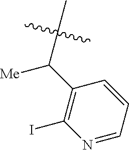 [SMILES: *C(C)c1cccnc1I] | R1-, R2-690 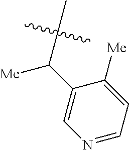 [SMILES: *C(C)c1cnccc1C] | R1-, R2-691 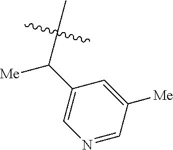 [SMILES: *C(C)c1cncc(C)c1] |
| R1-, R2-692 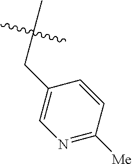 [SMILES: *Cc1ccc(C)nc1] | R1-, R2-693 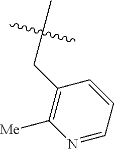 [SMILES: *Cc1cccnc1C] | R1-, R2-694 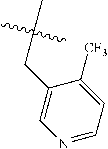 [SMILES: *Cc1cnccc1C(F)(F)F] | R1-, R2-695 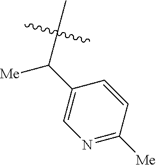 [SMILES: *C(C)c1ccc(C)nc1] | R1-, R2-696 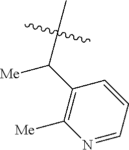 [SMILES: *C(C)c1cccnc1C] | R1-, R2-697 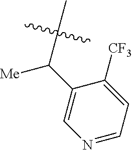 [SMILES: *C(C)c1cnccc1C(F)(F)F] |
| R1-, R2-698 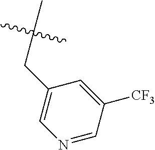 [SMILES: *Cc1cncc(C(F)(F)F)c1] | R1-, R2-699 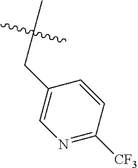 [SMILES: *Cc1ccc(C(F)(F)F)nc1] | R1-, R2-700 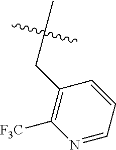 [SMILES: *Cc1cccnc1C(F)(F)F] | R1-, R2-701 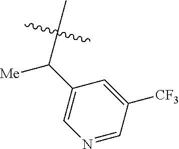 [SMILES: *C(C)c1cncc(C(F)(F)F)c1] | R1-, R2-702 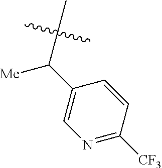 [SMILES: *C(C)c1ccc(C(F)(F)F)nc1] | R1-, R2-703 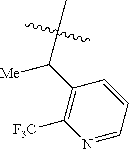 [SMILES: *C(C)c1cccnc1C(F)(F)F] |
| R1-, R2-704 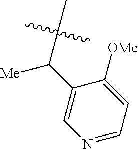 [SMILES: *C(C)c1cnccc1OC] | R1-, R2-705 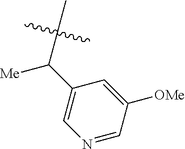 [SMILES: *C(C)c1cncc(OC)c1] | R1-, R2-706 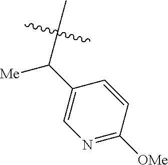 [SMILES: *C(C)c1ccc(OC)nc1] | R1-, R2-707 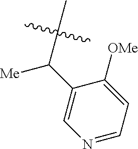 [SMILES: *C(C)c1cnccc1OC] | R1-, R2-708 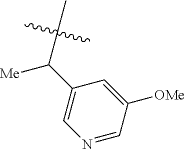 [SMILES: *C(C)c1cncc(OC)c1] | R1-, R2-709 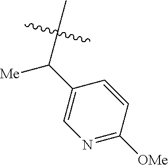 [SMILES: *C(C)c1ccc(OC)nc1] |
| R1-, R2-710 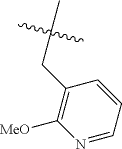 [SMILES: *Cc1cccnc1OC] | R1-, R2-711 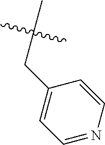 [SMILES: *Cc1ccncc1] | R1-, R2-712 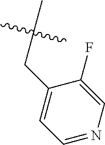 [SMILES: *Cc1ccncc1F] | R1-, R2-713 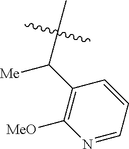 [SMILES: *C(C)c1cccnc1OC] | R1-, R2-714 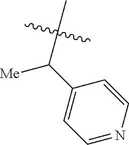 [SMILES: *C(C)c1ccncc1] | R1-, R2-715 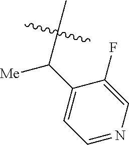 [SMILES: *C(C)c1ccncc1F] |
| R1-, R2-716 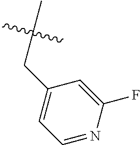 [SMILES: *Cc1ccnc(F)c1] | R1-, R2-717 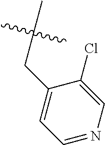 [SMILES: *Cc1ccncc1Cl] | R1-, R2-718 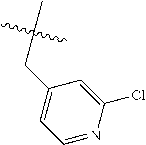 [SMILES: *Cc1ccnc(Cl)c1] | R1-, R2-719 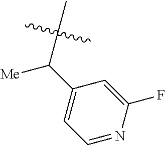 [SMILES: *C(C)c1ccnc(F)c1] | R1-, R2-720 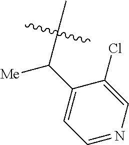 [SMILES: *C(C)c1ccncc1Cl] | R1-, R2-721 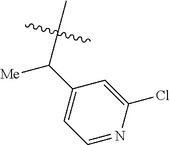 [SMILES: *C(C)c1ccnc(Cl)c1] |
| R1-, R2-722 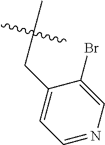 [SMILES: *Cc1ccncc1Br] | R1-, R2-723 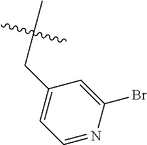 [SMILES: *Cc1ccnc(Br)c1] | R1-, R2-724 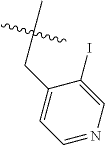 [SMILES: *Cc1ccncc1I] | R1-, R2-725 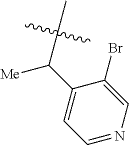 [SMILES: *C(C)c1ccncc1Br] | R1-, R2-726 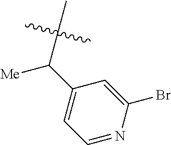 [SMILES: *C(C)c1ccnc(Br)c1] | R1-, R2-727 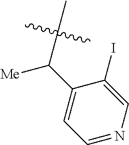 [SMILES: *C(C)c1ccncc1I] |
| R1-, R2-728 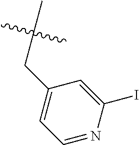 [SMILES: *Cc1ccnc(I)c1] | R1-, R2-729 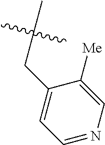 [SMILES: *Cc1ccncc1C] | R1-, R2-730 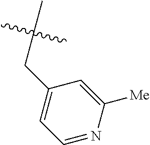 [SMILES: *Cc1ccnc(C)c1] | R1-, R2-731 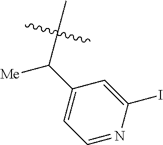 [SMILES: *C(C)c1ccnc(I)c1] | R1-, R2-732 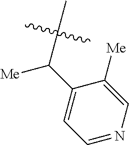 [SMILES: *C(C)c1ccncc1C] | R1-, R2-733 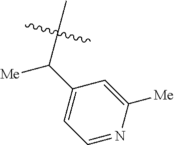 [SMILES: *C(C)c1ccnc(C)c1] |
| R1-, R2-734 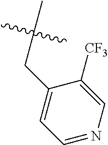 [SMILES: *Cc1ccncc1C(F)(F)F] | R1-, R2-735 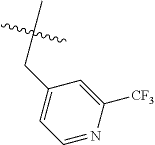 [SMILES: *Cc1ccnc(C(F)(F)F)c1] | R1-, R2-736 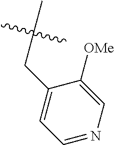 [SMILES: *Cc1ccncc1OC] | R1-, R2-737 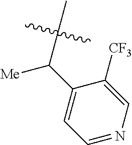 [SMILES: *C(C)c1ccncc1C(F)(F)F] | R1-, R2-738 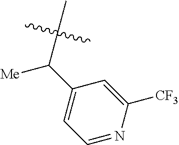 [SMILES: *C(C)c1ccnc(C(F)(F)F)c1] | R1-, R2-739 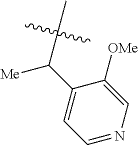 [SMILES: *C(C)c1ccncc1OC] |
| R1-, R2-740 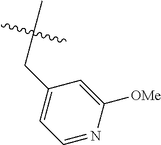 [SMILES: *Cc1ccnc(OC)c1] | R1-, R2-741 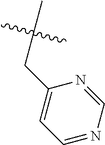 [SMILES: *Cc1ccncn1] | R1-, R2-742 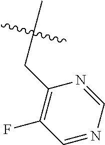 [SMILES: *Cc1ncncc1F] | R1-, R2-743 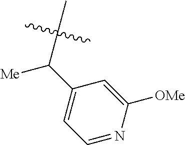 [SMILES: *C(C)c1ccnc(OC)c1] | R1-, R2-744 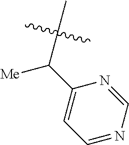 [SMILES: *C(C)c1ccncn1] | R1-, R2-745 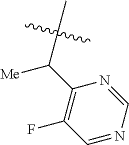 [SMILES: *C(C)c1ncncc1F] |
| R1-, R2-746 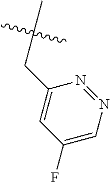 [SMILES: *Cc1cc(F)cnn1] | R1-, R2-747 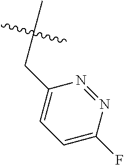 [SMILES: *Cc1ccc(F)nn1] | R1-, R2-748 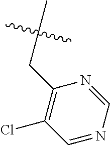 [SMILES: *Cc1ncncc1Cl] | R1-, R2-749 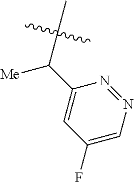 [SMILES: *C(C)c1cc(F)cnn1] | R1-, R2-750 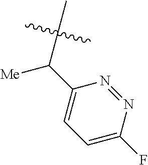 [SMILES: *C(C)c1ccc(F)nn1] | R1-, R2-751 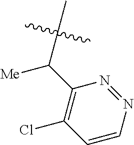 [SMILES: *C(C)c1nnccc1Cl] |
| R1-, R2-752 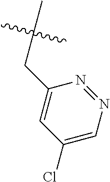 [SMILES: *Cc1cc(Cl)cnn1] | R1-, R2-753 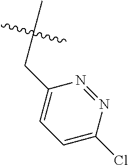 [SMILES: *Cc1ccc(Cl)nn1] | R1-, R2-754 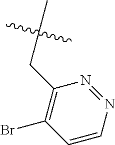 [SMILES: *Cc1nnccc1Br] | R1-, R2-755 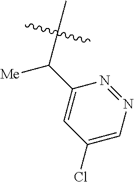 [SMILES: *C(C)c1cc(Cl)cnn1] | R1-, R2-756 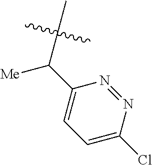 [SMILES: *C(C)c1ccc(Cl)nn1] | R1-, R2-757 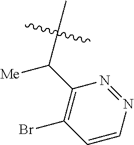 [SMILES: *C(C)c1nnccc1Br] |
| R1-, R2-758 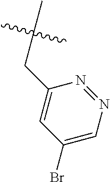 [SMILES: *Cc1cc(Br)cnn1] | R1-, R2-759 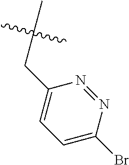 [SMILES: *Cc1ccc(Br)nn1] | R1-, R2-760 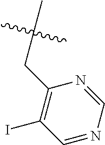 [SMILES: *Cc1ncncc1I] | R1-, R2-761 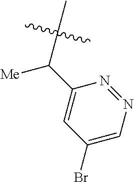 [SMILES: *C(C)c1cc(Br)cnn1] | R1-, R2-762 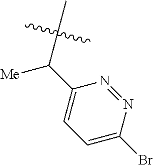 [SMILES: *C(C)c1ccc(Br)nn1] | R1-, R2-763 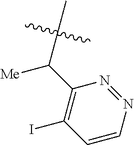 [SMILES: *C(C)c1nnccc1I] |
| R1-, R2-764 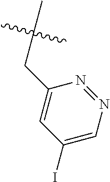 [SMILES: *Cc1cc(I)cnn1] | R1-, R2-765 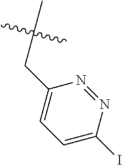 [SMILES: *Cc1ccc(I)nn1] | R1-, R2-766 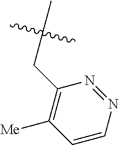 [SMILES: *Cc1nnccc1C] | R1-, R2-767 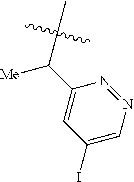 [SMILES: *C(C)c1cc(I)cnn1] | R1-, R2-768 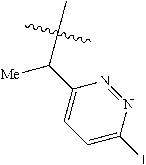 [SMILES: *C(C)c1ccc(I)nn1] | R1-, R2-769 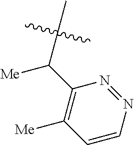 [SMILES: *C(C)c1nnccc1C] |
| R1-, R2-770 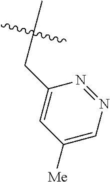 [SMILES: *Cc1cc(C)cnn1] | R1-, R2-771 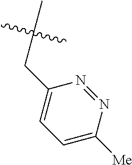 [SMILES: *Cc1ccc(C)nn1] | R1-, R2-772 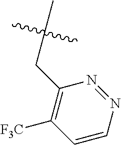 [SMILES: *Cc1nnccc1C(F)(F)F] | R1-, R2-773 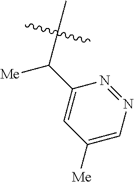 [SMILES: *C(C)c1cc(C)cnn1] | R1-, R2-774 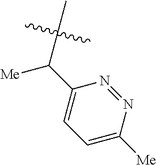 [SMILES: *C(C)c1ccc(C)nn1] | R1-, R2-775 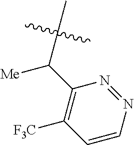 [SMILES: *C(C)c1nnccc1C(F)(F)F] |
| R1-, R2-776 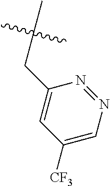 [SMILES: *Cc1cc(C(F)(F)F)cnn1] | R1-, R2-777 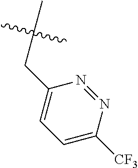 [SMILES: *Cc1ccc(C(F)(F)F)nn1] | R1-, R2-778 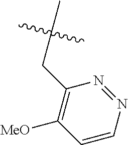 [SMILES: *Cc1nnccc1OC] | R1-, R2-779 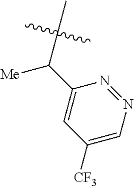 [SMILES: *C(C)c1cc(C(F)(F)F)cnn1] | R1-, R2-780 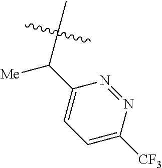 [SMILES: *C(C)c1ccc(C(F)(F)F)nn1] | R1-, R2-781 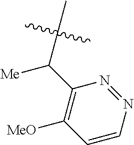 [SMILES: *C(C)c1nnccc1OC] |
| R1-, R2-782 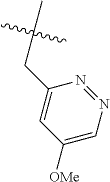 [SMILES: *Cc1cc(OC)cnn1] | R1-, R2-783 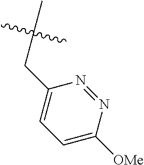 [SMILES: *Cc1ccc(OC)nn1] | R1-, R2-784 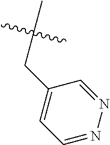 [SMILES: *Cc1ccnnc1] | R1-, R2-785 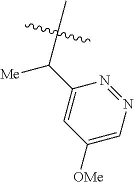 [SMILES: *C(C)c1cc(OC)cnn1] | R1-, R2-786 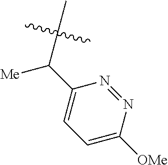 [SMILES: *C(C)c1ccc(OC)nn1] | R1-, R2-787 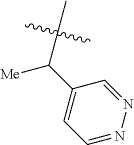 [SMILES: *C(C)c1ccnnc1] |
| R1-, R2-788 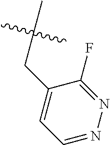 [SMILES: *Cc1ccnnc1F] | R1-, R2-789 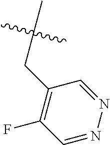 [SMILES: *Cc1cnncc1F] | R1-, R2-790 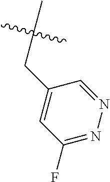 [SMILES: *Cc1cnnc(F)c1] | R1-, R2-791 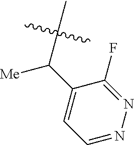 [SMILES: *C(C)c1ccnnc1F] | R1-, R2-792 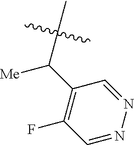 [SMILES: *C(C)c1cnncc1F] | R1-, R2-793 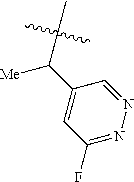 [SMILES: *C(C)c1cnnc(F)c1] |
| R1-, R2-794 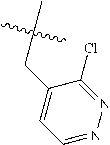 [SMILES: *Cc1ccnnc1Cl] | R1-, R2-795 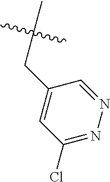 [SMILES: *Cc1cnnc(Cl)c1] | R1-, R2-796 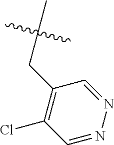 [SMILES: *Cc1cnncc1Cl] | R1-, R2-797 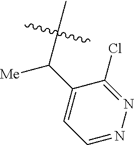 [SMILES: *C(C)c1ccnnc1Cl] | R1-, R2-798 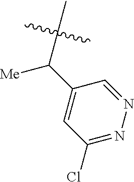 [SMILES: *C(C)c1cnnc(Cl)c1] | R1-, R2-799 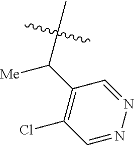 [SMILES: *C(C)c1cnncc1Cl] |
| R1-, R2-800 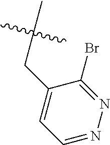 [SMILES: *Cc1ccnnc1Br] | R1-, R2-801 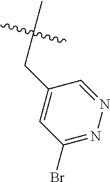 [SMILES: *Cc1cnnc(Br)c1] | R1-, R2-802 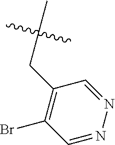 [SMILES: *Cc1cnncc1Br] | R1-, R2-803 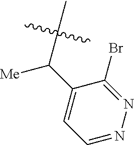 [SMILES: *C(C)c1ccnnc1Br] | R1-, R2-804 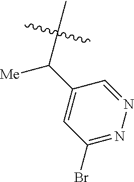 [SMILES: *C(C)c1cnnc(Br)c1] | R1-, R2-805 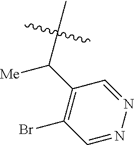 [SMILES: *C(C)c1cnncc1Br] |
| R1-, R2-806 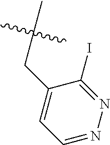 [SMILES: *Cc1ccnnc1I] | R1-, R2-807 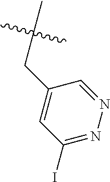 [SMILES: *Cc1cnnc(I)c1] | R1-, R2-808 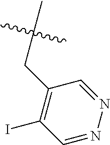 [SMILES: *Cc1cnncc1I] | R1-, R2-809 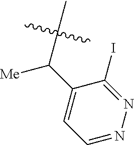 [SMILES: *C(C)c1ccnnc1I] | R1-, R2-810 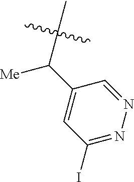 [SMILES: *C(C)c1cnnc(I)c1] | R1-, R2-811 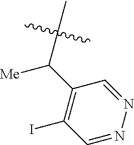 [SMILES: *C(C)c1cnncc1I] |
| R1-, R2-812 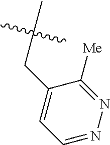 [SMILES: *Cc1ccnnc1C] | R1-, R2-813 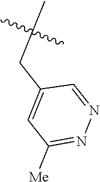 [SMILES: *Cc1cnnc(C)c1] | R1-, R2-814 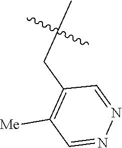 [SMILES: *Cc1cnncc1C] | R1-, R2-815 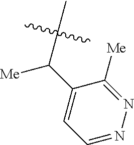 [SMILES: *C(C)c1ccnnc1C] | R1-, R2-816 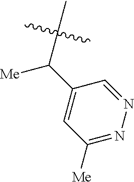 [SMILES: *C(C)c1cnnc(C)c1] | R1-, R2-817 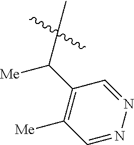 [SMILES: *C(C)c1cnncc1C] |
| R1-, R2-818 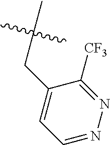 [SMILES: *Cc1ccnnc1C(F)(F)F] | R1-, R2-819 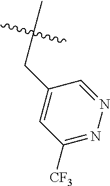 [SMILES: *Cc1cnnc(C(F)(F)F)c1] | R1-, R2-820 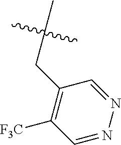 [SMILES: *Cc1cnncc1C(F)(F)F] | R1-, R2-821 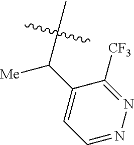 [SMILES: *C(C)c1ccnnc1C(F)(F)F] | R1-, R2-822 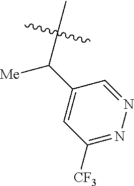 [SMILES: *C(C)c1cnnc(C(F)(F)F)c1] | R1-, R2-823 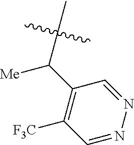 [SMILES: *C(C)c1cnncc1C(F)(F)F] |
| R1-, R2-824 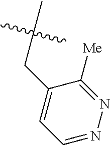 [SMILES: *Cc1ccnnc1C] | R1-, R2-825 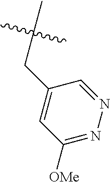 [SMILES: *Cc1cnnc(OC)c1] | R1-, R2-826 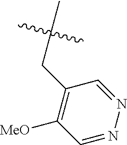 [SMILES: *Cc1cnncc1OC] | R1-, R2-827 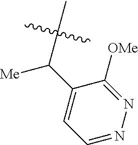 [SMILES: *C(C)c1ccnnc1OC] | R1-, R2-828 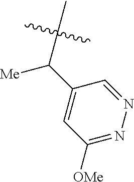 [SMILES: *C(C)c1cnnc(OC)c1] | R1-, R2-829 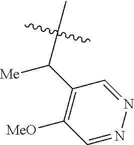 [SMILES: *C(C)c1cnncc1OC] |
| R1-, R2-830 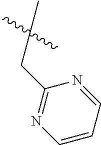 [SMILES: *Cc1ncccn1] | R1-, R2-831 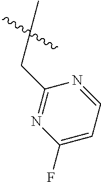 [SMILES: *Cc1nccc(F)n1] | R1-, R2-832 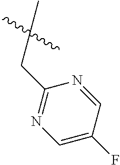 [SMILES: *Cc1ncc(F)cn1] | R1-, R2-833 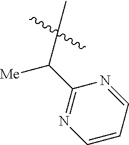 [SMILES: *C(C)c1ncccn1] | R1-, R2-834 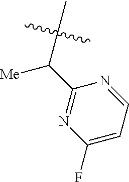 [SMILES: *C(C)c1nccc(F)n1] | R1-, R2-835 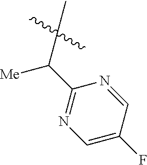 [SMILES: *C(C)c1ncc(F)cn1] |
| R1-, R2-836 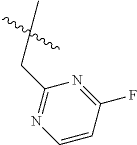 [SMILES: *Cc1nccc(F)n1] | R1-, R2-837 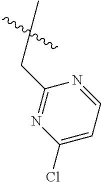 [SMILES: *Cc1nccc(Cl)n1] | R1-, R2-838 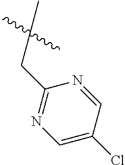 [SMILES: *Cc1ncc(Cl)cn1] | R1-, R2-839 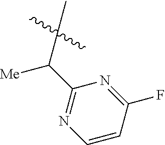 [SMILES: *C(C)c1nccc(F)n1] | R1-, R2-840 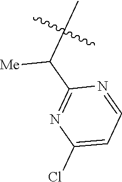 [SMILES: *C(C)c1nccc(Cl)n1] | R1-, R2-841 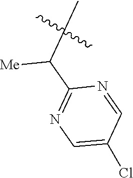 [SMILES: *C(C)c1ncc(Cl)cn1] |
| R1-, R2-842 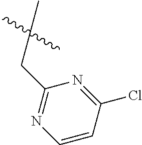 [SMILES: *Cc1nccc(Cl)n1] | R1-, R2-843 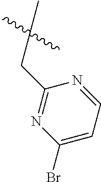 [SMILES: *Cc1nccc(Br)n1] | R1-, R2-844 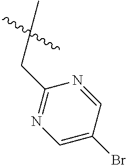 [SMILES: *Cc1ncc(Br)cn1] | R1-, R2-845 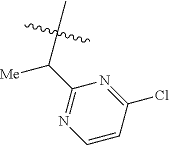 [SMILES: *C(C)c1nccc(Cl)n1] | R1-, R2-846 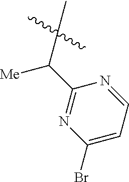 [SMILES: *C(C)c1nccc(Br)n1] | R1-, R2-847 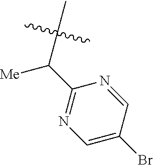 [SMILES: *C(C)c1ncc(Br)cn1] |
| R1-, R2-848 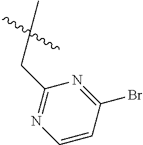 [SMILES: *Cc1nccc(Br)n1] | R1-, R2-849 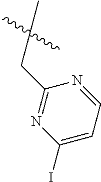 [SMILES: *Cc1nccc(I)n1] | R1-, R2-850 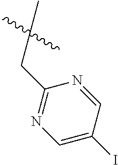 [SMILES: *Cc1ncc(I)cn1] | R1-, R2-851 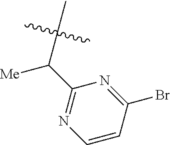 [SMILES: *C(C)c1nccc(Br)n1] | R1-, R2-852 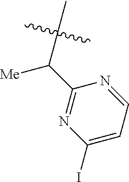 [SMILES: *C(C)c1nccc(I)n1] | R1-, R2-853 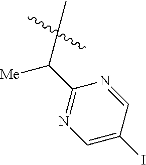 [SMILES: *C(C)c1ncc(I)cn1] |
| R1-, R2-854 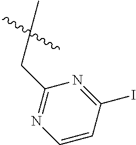 [SMILES: *Cc1nccc(I)n1] | R1-, R2-855 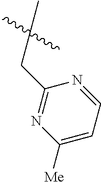 [SMILES: *Cc1nccc(C)n1] | R1-, R2-856 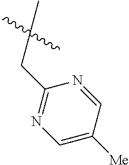 [SMILES: *Cc1ncc(C)cn1] | R1-, R2-857 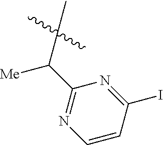 [SMILES: *C(C)c1nccc(I)n1] | R1-, R2-858 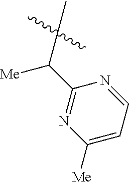 [SMILES: *C(C)c1nccc(C)n1] | R1-, R2-859 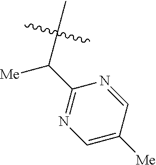 [SMILES: *C(C)c1ncc(C)cn1] |
| R1-, R2-860 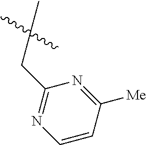 [SMILES: *Cc1nccc(C)n1] | R1-, R2-861 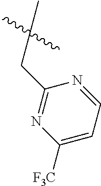 [SMILES: *Cc1nccc(C(F)(F)F)n1] | R1-, R2-862 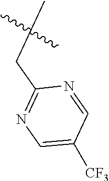 [SMILES: *Cc1ncc(C(F)(F)F)cn1] | R1-, R2-863 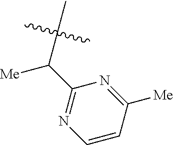 [SMILES: *C(C)c1nccc(C)n1] | R1-, R2-864 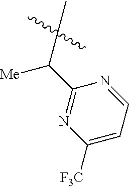 [SMILES: *C(C)c1nccc(C(F)(F)F)n1] | R1-, R2-865 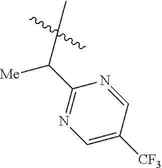 [SMILES: *C(C)c1ncc(C(F)(F)F)cn1] |
| R1-, R2-866 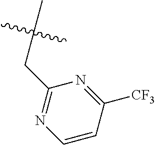 [SMILES: *Cc1nccc(C(F)(F)F)n1] | R1-, R2-867 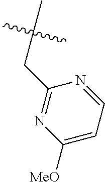 [SMILES: *Cc1nccc(OC)n1] | R1-, R2-868 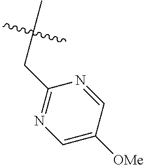 [SMILES: *Cc1ncc(OC)cn1] | R1-, R2-869 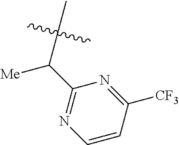 [SMILES: *C(C)c1nccc(C(F)(F)F)n1] | R1-, R2-870 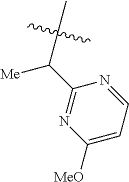 [SMILES: *C(C)c1nccc(OC)n1] | R1-, R2-871 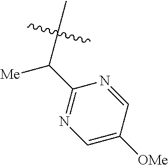 [SMILES: *C(C)c1ncc(OC)cn1] |
| R1-, R2-872 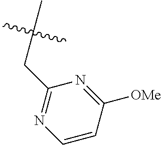 [SMILES: *Cc1nccc(OC)n1] | R1-, R2-873 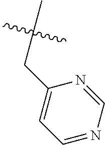 [SMILES: *Cc1ccncn1] | R1-, R2-874 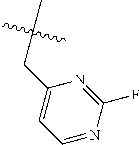 [SMILES: *Cc1ccnc(F)n1] | R1-, R2-875 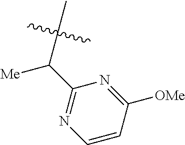 [SMILES: *C(C)c1nccc(OC)n1] | R1-, R2-876 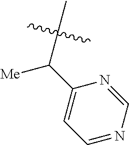 [SMILES: *C(C)c1ccncn1] | R1-, R2-877 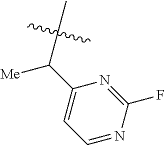 [SMILES: *C(C)c1ccnc(F)n1] |
| R1-, R2-878 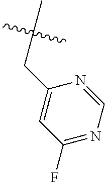 [SMILES: *Cc1cc(F)ncn1] | R1-, R2-879 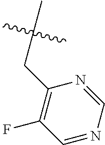 [SMILES: *Cc1ncncc1F] | R1-, R2-880 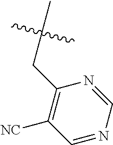 [SMILES: *Cc1ncncc1C#N] | R1-, R2-881 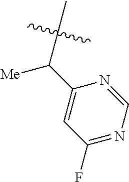 [SMILES: *C(C)c1cc(F)ncn1] | R1-, R2-882 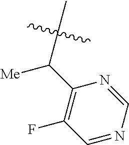 [SMILES: *C(C)c1ncncc1F] | R1-, R2-883 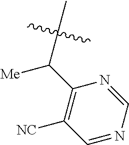 [SMILES: *C(C)c1ncncc1C#N] |
| R1-, R2-884 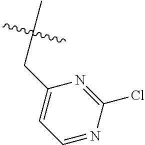 [SMILES: *Cc1ccnc(Cl)n1] | R1-, R2-885 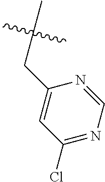 [SMILES: *Cc1cc(Cl)ncn1] | R1-, R2-886 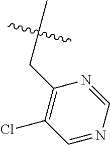 [SMILES: *Cc1ncncc1Cl] | R1-, R2-887 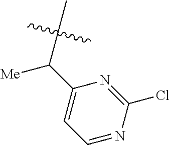 [SMILES: *C(C)c1ccnc(Cl)n1] | R1-, R2-888 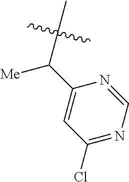 [SMILES: *C(C)c1cc(Cl)ncn1] | R1-, R2-889 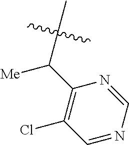 [SMILES: *C(C)c1ncncc1Cl] |
| R1-, R2-890 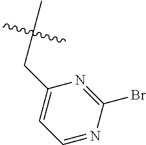 [SMILES: *Cc1ccnc(Br)n1] | R1-, R2-891 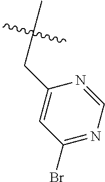 [SMILES: *Cc1cc(Br)ncn1] | R1-, R2-892 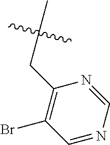 [SMILES: *Cc1ncncc1Br] | R1-, R2-893 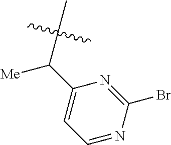 [SMILES: *C(C)c1ccnc(Br)n1] | R1-, R2-894 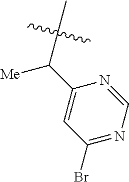 [SMILES: *C(C)c1cc(Br)ncn1] | R1-, R2-895 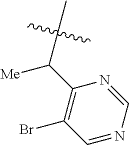 [SMILES: *C(C)c1ncncc1Br] |
| R1-, R2-896 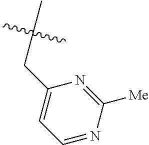 [SMILES: *Cc1ccnc(C)n1] | R1-, R2-897 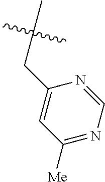 [SMILES: *Cc1cc(C)ncn1] | R1-, R2-898 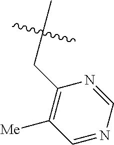 [SMILES: *Cc1ncncc1C] | R1-, R2-899 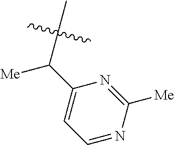 [SMILES: *C(C)c1ccnc(C)n1] | R1-, R2-900 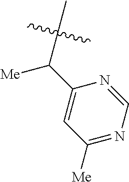 [SMILES: *C(C)c1cc(C)ncn1] | R1-, R2-901 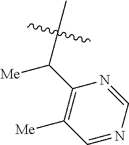 [SMILES: *C(C)c1ncncc1C] |
| R1-, R2-902 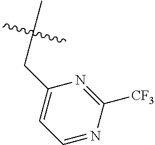 [SMILES: *Cc1ccnc(C(F)(F)F)n1] | R1-, R2-903 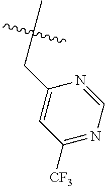 [SMILES: *Cc1cc(C(F)(F)F)ncn1] | R1-, R2-904 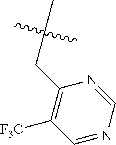 [SMILES: *Cc1ncncc1C(F)(F)F] | R1-, R2-905 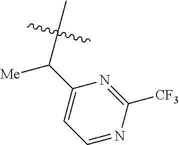 [SMILES: *C(C)c1ccnc(C(F)(F)F)n1] | R1-, R2-906 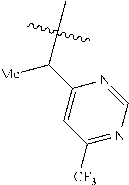 [SMILES: *C(C)c1cc(C(F)(F)F)ncn1] | R1-, R2-907 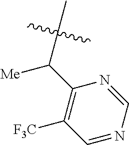 [SMILES: *C(C)c1ncncc1C(F)(F)F] |
| R1-, R2-908 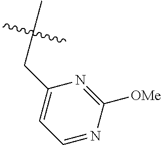 [SMILES: *Cc1ccnc(OC)n1] | R1-, R2-909 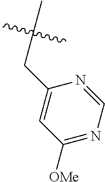 [SMILES: *Cc1cc(OC)ncn1] | R1-, R2-910 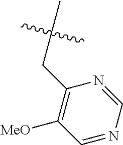 [SMILES: *Cc1ncncc1OC] | R1-, R2-911 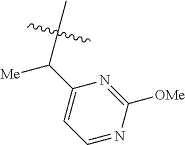 [SMILES: *C(C)c1ccnc(OC)n1] | R1-, R2-912 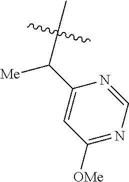 [SMILES: *C(C)c1cc(OC)ncn1] | R1-, R2-913 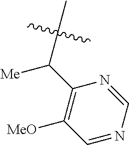 [SMILES: *C(C)c1ncncc1OC] |
| R1-, R2-914 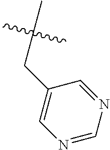 [SMILES: *Cc1cncnc1] | R1-, R2-915 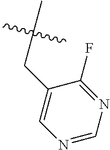 [SMILES: *Cc1cncnc1F] | R1-, R2-916 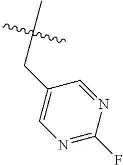 [SMILES: *Cc1cnc(F)nc1] | R1-, R2-917 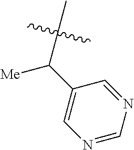 [SMILES: *C(C)c1cncnc1] | R1-, R2-918 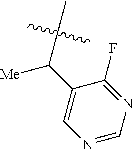 [SMILES: *C(C)c1cncnc1F] | R1-, R2-919 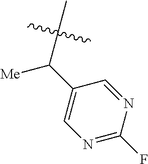 [SMILES: *C(C)c1cnc(F)nc1] |
| R1-, R2-920 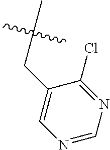 [SMILES: *Cc1cncnc1Cl] | R1-, R2-921 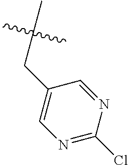 [SMILES: *Cc1cnc(Cl)nc1] | R1-, R2-922 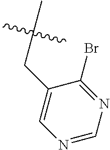 [SMILES: *Cc1cncnc1Br] | R1-, R2-923 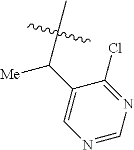 [SMILES: *C(C)c1cncnc1Cl] | R1-, R2-924 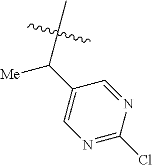 [SMILES: *C(C)c1cnc(Cl)nc1] | R1-, R2-925 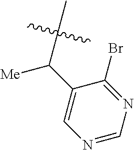 [SMILES: *C(C)c1cncnc1Br] |
| R1-, R2-926 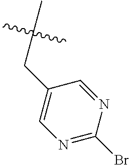 [SMILES: *Cc1cnc(Br)nc1] | R1-, R2-927 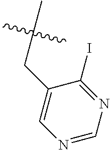 [SMILES: *Cc1cncnc1I] | R1-, R2-928 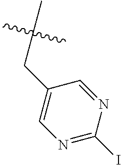 [SMILES: *Cc1cnc(I)nc1] | R1-, R2-929 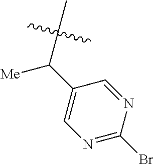 [SMILES: *C(C)c1cnc(Br)nc1] | R1-, R2-930 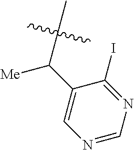 [SMILES: *C(C)c1cncnc1I] | R1-, R2-931 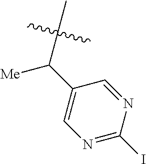 [SMILES: *C(C)c1cnc(I)nc1] |
| R1-, R2-932 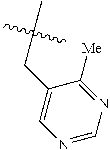 [SMILES: *Cc1cncnc1C] | R1-, R2-933 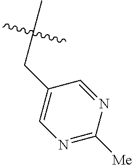 [SMILES: *Cc1cnc(C)nc1] | R1-, R2-934 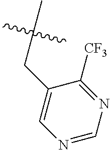 [SMILES: *Cc1cncnc1C(F)(F)F] | R1-, R2-935 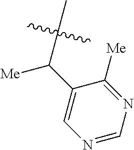 [SMILES: *C(C)c1cncnc1C] | R1-, R2-936 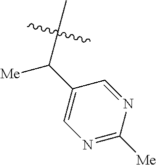 [SMILES: *C(C)c1cnc(C)nc1] | R1-, R2-937 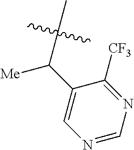 [SMILES: *C(C)c1cncnc1C(F)(F)F] |
| R1-, R2-938 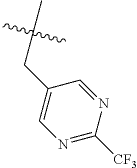 [SMILES: *Cc1cnc(C(F)(F)F)nc1] | R1-, R2-939 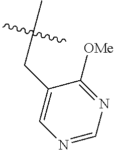 [SMILES: *Cc1cncnc1OC] | R1-, R2-940 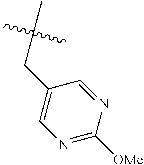 [SMILES: *Cc1cnc(OC)nc1] | R1-, R2-941 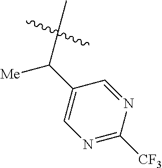 [SMILES: *C(C)c1cnc(C(F)(F)F)nc1] | R1-, R2-942 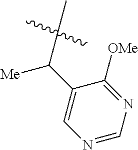 [SMILES: *C(C)c1cncnc1OC] | R1-, R2-943 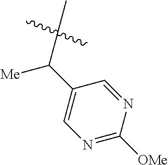 [SMILES: *C(C)c1cnc(OC)nc1] |
| R1-, R2-944 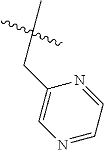 [SMILES: *Cc1cnccn1] | R1-, R2-945 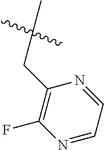 [SMILES: *Cc1nccnc1F] | R1-, R2-946 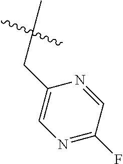 [SMILES: *Cc1cnc(F)cn1] | R1-, R2-947 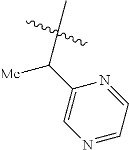 [SMILES: *C(C)c1cnccn1] | R1-, R2-948 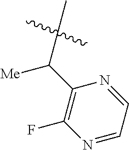 [SMILES: *C(C)c1nccnc1F] | R1-, R2-949 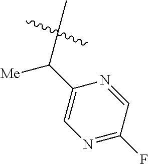 [SMILES: *C(C)c1cnc(F)cn1] |
| R1-, R2-950 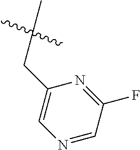 [SMILES: *Cc1cncc(F)n1] | R1-, R2-951 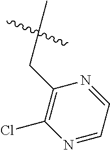 [SMILES: *Cc1nccnc1Cl] | R1-, R2-952 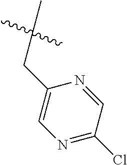 [SMILES: *Cc1cnc(Cl)cn1] | R1-, R2-953 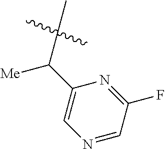 [SMILES: *C(C)c1cncc(F)n1] | R1-, R2-954 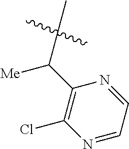 [SMILES: *C(C)c1nccnc1Cl] | R1-, R2-955 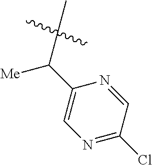 [SMILES: *C(C)c1cnc(Cl)cn1] |
| R1-, R2-956 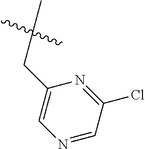 [SMILES: *Cc1cncc(Cl)n1] | R1-, R2-957 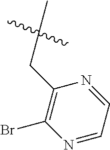 [SMILES: *Cc1nccnc1Br] | R1-, R2-958 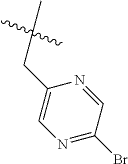 [SMILES: *Cc1cnc(Br)cn1] | R1-, R2-959 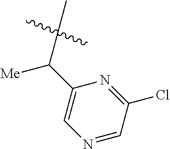 [SMILES: *C(C)c1cncc(Cl)n1] | R1-, R2-960 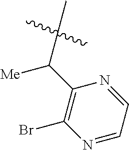 [SMILES: *C(C)c1nccnc1Br] | R1-, R2-961 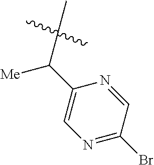 [SMILES: *C(C)c1cnc(Br)cn1] |
| R1-, R2-962 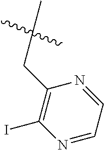 [SMILES: *Cc1nccnc1I] | R1-, R2-963 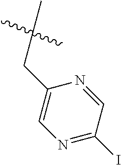 [SMILES: *Cc1cnc(I)cn1] | R1-, R2-964 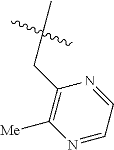 [SMILES: *Cc1nccnc1C] | R1-, R2-965 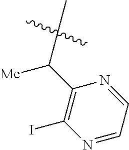 [SMILES: *C(C)c1nccnc1I] | R1-, R2-966 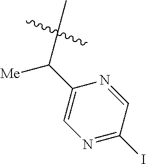 [SMILES: *C(C)c1cnc(I)cn1] | R1-, R2-967 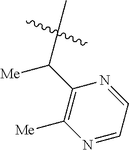 [SMILES: *C(C)c1nccnc1C] |
| R1-, R2-968 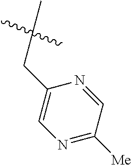 [SMILES: *Cc1cnc(C)cn1] | R1-, R2-969 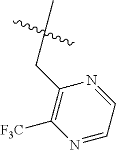 [SMILES: *Cc1nccnc1C(F)(F)F] | R1-, R2-970 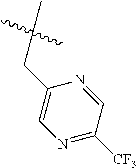 [SMILES: *Cc1cnc(C(F)(F)F)cn1] | R1-, R2-971 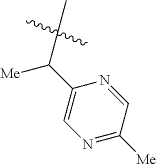 [SMILES: *C(C)c1cnc(C)cn1] | R1-, R2-972 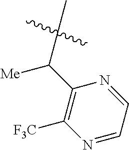 [SMILES: *C(C)c1nccnc1C(F)(F)F] | R1-, R2-973 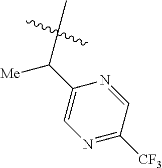 [SMILES: *C(C)c1cnc(C(F)(F)F)cn1] |
| R1-, R2-974 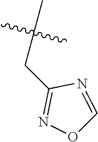 [SMILES: *Cc1ncon1] | R1-, R2-975 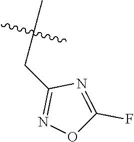 [SMILES: *Cc1noc(F)n1] | R1-, R2-976 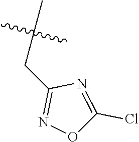 [SMILES: *Cc1noc(Cl)n1] | R1-, R2-977 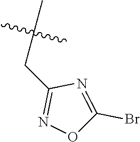 [SMILES: *Cc1noc(Br)n1] | R1-, R2-978 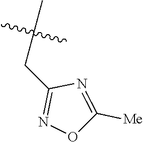 [SMILES: *Cc1noc(C)n1] | R1-, R2-979 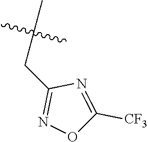 [SMILES: *Cc1noc(C(F)(F)F)n1] |
| R1-, R2-980 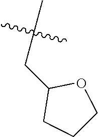 [SMILES: *CC1CCCO1] | R1-, R2-981 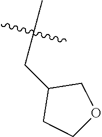 [SMILES: *CC1CCOC1] | R1-, R2-982 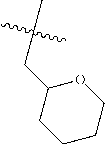 [SMILES: *CC1CCCCO1] | R1-, R2-983 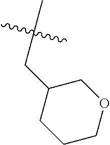 [SMILES: *CC1CCCOC1] | R1-, R2-984 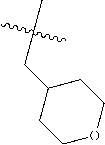 [SMILES: *CC1CCOCC1] | R1-, R2-985 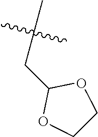 [SMILES: *CC1OCCO1] |
| R1-, R2-986 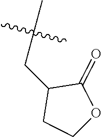 [SMILES: *CC1CCOC1=O] | R1-, R2-987 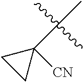 [SMILES: *C1(C#N)CC1] | R1-, R2-988 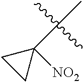 [SMILES: *C1([N+](=O)[O-])CC1] | R1-, R2-989 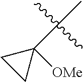 [SMILES: *C1(OC)CC1] | R1-, R2-990 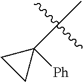 [SMILES: *C1(c2ccccc2)CC1] | R1-, R2-991 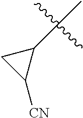 [SMILES: *C1CC1C#N] |
| R1-, R2-992 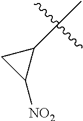 [SMILES: *C1CC1[N+](=O)[O-]] | R1-, R2-993 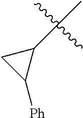 [SMILES: *C1CC1c1ccccc1] | R1-, R2-994 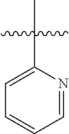 [SMILES: *c1ccccn1] | R1-, R2-995 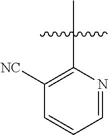 [SMILES: *c1ncccc1C#N] | R1-, R2-996 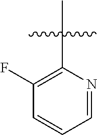 [SMILES: *c1ncccc1F] | R1-, R2-997 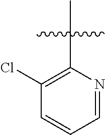 [SMILES: *c1ncccc1Cl] |
| R1-, R2-998 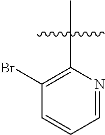 [SMILES: *c1ncccc1Br] | R1-, R2-999 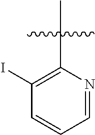 [SMILES: *c1ncccc1I] | R1-, R2-1000 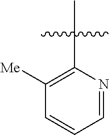 [SMILES: *c1ncccc1C] | R1-, R2-1001 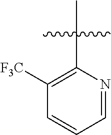 [SMILES: *c1ncccc1C(F)(F)F] | R1-, R2-1002 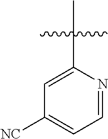 [SMILES: *c1cc(C#N)ccn1] | R1-, R2-1003 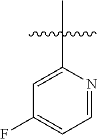 [SMILES: *c1cc(F)ccn1] |
| R1-, R2-1004 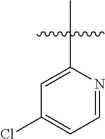 [SMILES: *c1cc(Cl)ccn1] | R1-, R2-1005 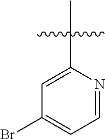 [SMILES: *c1cc(Br)ccn1] | R1-, R2-1006 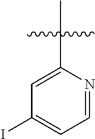 [SMILES: *c1cc(I)ccn1] | R1-, R2-1007 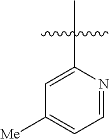 [SMILES: *c1cc(C)ccn1] | R1-, R2-1008 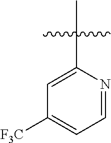 [SMILES: *c1cc(C(F)(F)F)ccn1] | R1-, R2-1009 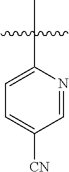 [SMILES: *c1ccc(C#N)cn1] |
| R1-, R2-1010 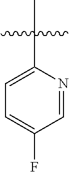 [SMILES: *c1ccc(F)cn1] | R1-, R2-1011 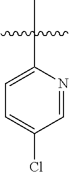 [SMILES: *c1ccc(Cl)cn1] | R1-, R2-1012 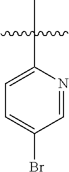 [SMILES: *c1ccc(Br)cn1] | R1-, R2-1013 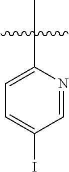 [SMILES: *c1ccc(I)cn1] | R1-, R2-1014 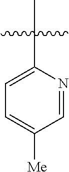 [SMILES: *c1ccc(C)cn1] | R1-, R2-1015 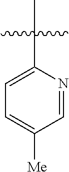 [SMILES: *c1ccc(C)cn1] |
| R1-, R2-1016 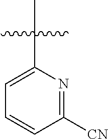 [SMILES: *c1cccc(C#N)n1] | R1-, R2-1017 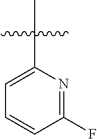 [SMILES: *c1cccc(F)n1] | R1-, R2-1018 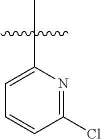 [SMILES: *c1cccc(Cl)n1] | R1-, R2-1019 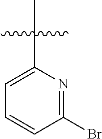 [SMILES: *c1cccc(Br)n1] | R1-, R2-1020 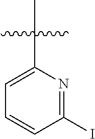 [SMILES: *c1cccc(I)n1] | R1-, R2-1021 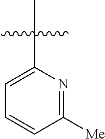 [SMILES: *c1cccc(C)n1] |
| R1-, R2-1022 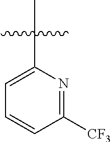 [SMILES: *c1cccc(C(F)(F)F)n1] | R1-, R2-1023 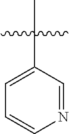 [SMILES: *c1cccnc1] | R1-, R2-1024 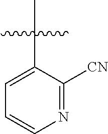 [SMILES: *c1cccnc1C#N] | R1-, R2-1025 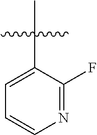 [SMILES: *c1cccnc1F] | R1-, R2-1026 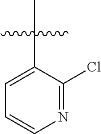 [SMILES: *c1cccnc1Cl] | R1-, R2-1027 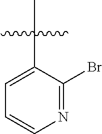 [SMILES: *c1cccnc1Br] |
| R1-, R2-1028 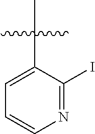 [SMILES: *c1cccnc1I] | R1-, R2-1029 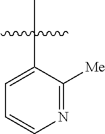 [SMILES: *c1cccnc1C] | R1-, R2-1030 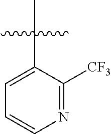 [SMILES: *c1cccnc1C(F)(F)F] | R1-, R2-1031 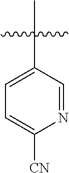 [SMILES: *c1ccc(C#N)nc1] | R1-, R2-1032 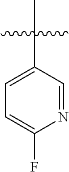 [SMILES: *c1ccc(F)nc1] | R1-, R2-1033 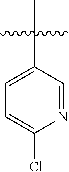 [SMILES: *c1ccc(Cl)nc1] |
| R1-, R2-1034 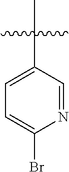 [SMILES: *c1ccc(Br)nc1] | R1-, R2-1035 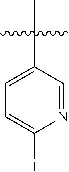 [SMILES: *c1ccc(I)nc1] | R1-, R2-1036 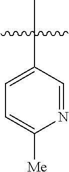 [SMILES: *c1ccc(C)nc1] | R1-, R2-1037 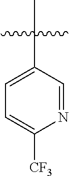 [SMILES: *c1ccc(C(F)(F)F)nc1] | R1-, R2-1038 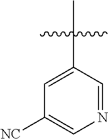 [SMILES: *c1cncc(C#N)c1] | R1-, R2-1039 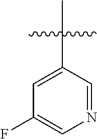 [SMILES: *c1cncc(F)c1] |
| R1-, R2-1040 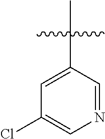 [SMILES: *c1cncc(Cl)c1] | R1-, R2-1041 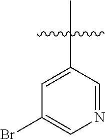 [SMILES: *c1cncc(Br)c1] | R1-, R2-1042 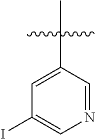 [SMILES: *c1cncc(I)c1] | R1-, R2-1043 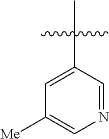 [SMILES: *c1cncc(C)c1] | R1-, R2-1044 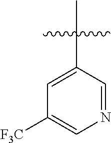 [SMILES: *c1cncc(C(F)(F)F)c1] | R1-, R2-1045 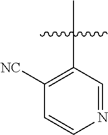 [SMILES: *c1cnccc1C#N] |
| R1-, R2-1046 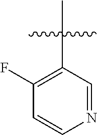 [SMILES: *c1cnccc1F] | R1-, R2-1047 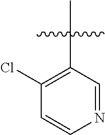 [SMILES: *c1cnccc1Cl] | R1-, R2-1048 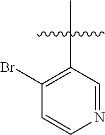 [SMILES: *c1cnccc1Br] | R1-, R2-1049 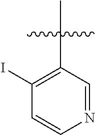 [SMILES: *c1cnccc1I] | R1-, R2-1050 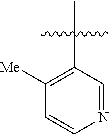 [SMILES: *c1cnccc1C] | R1-, R2-1051 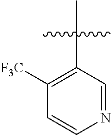 [SMILES: *c1cnccc1C(F)(F)F] |
| R1-, R2-1052 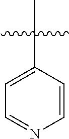 [SMILES: *c1ccncc1] | R1-, R2-1053 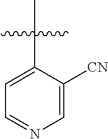 [SMILES: *c1ccncc1C#N] | R1-, R2-1054 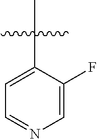 [SMILES: *c1ccncc1F] | R1-, R2-1055 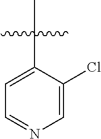 [SMILES: *c1ccncc1Cl] | R1-, R2-1056 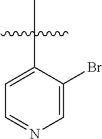 [SMILES: *c1ccncc1Br] | R1-, R2-1057 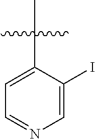 [SMILES: *c1ccncc1I] |
| R1-, R2-1058 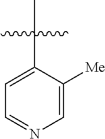 [SMILES: *c1ccncc1C] | R1-, R2-1059 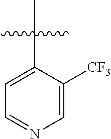 [SMILES: *c1ccncc1C(F)(F)F] | R1-, R2-1060 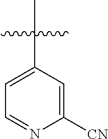 [SMILES: *c1ccnc(C#N)c1] | R1-, R2-1061 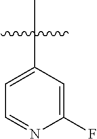 [SMILES: *c1ccnc(F)c1] | R1-, R2-1062 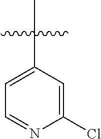 [SMILES: *c1ccnc(Cl)c1] | R1-, R2-1063 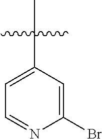 [SMILES: *c1ccnc(Br)c1] |
| R1-, R2-1064 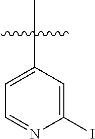 [SMILES: *c1ccnc(I)c1] | R1-, R2-1065 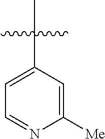 [SMILES: *c1ccnc(C)c1] | R1-, R2-1066 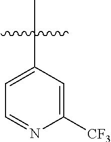 [SMILES: *c1ccnc(C(F)(F)F)c1] | R1-, R2-1067 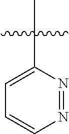 [SMILES: *c1cccnn1] | R1-, R2-1068 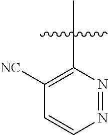 [SMILES: *c1nnccc1C#N] | R1-, R2-1069 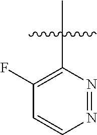 [SMILES: *c1nnccc1F] |
| R1-, R2-1070 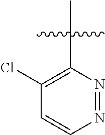 [SMILES: *c1nnccc1Cl] | R1-, R2-1071 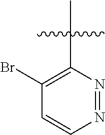 [SMILES: *c1nnccc1Br] | R1-, R2-1072 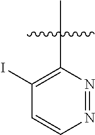 [SMILES: *c1nnccc1I] | R1-, R2-1073 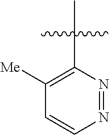 [SMILES: *c1nnccc1C] | R1-, R2-1074 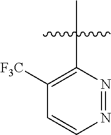 [SMILES: *c1nnccc1C(F)(F)F] | R1-, R2-1075 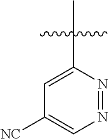 [SMILES: *c1cc(C#N)cnn1] |
| R1-, R2-1076 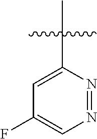 [SMILES: *c1cc(F)cnn1] | R1-, R2-1077 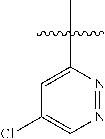 [SMILES: *c1cc(Cl)cnn1] | R1-, R2-1078 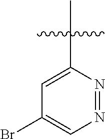 [SMILES: *c1cc(Br)cnn1] | R1-, R2-1079 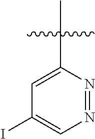 [SMILES: *c1cc(I)cnn1] | R1-, R2-1080 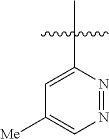 [SMILES: *c1cc(C)cnn1] | R1-, R2-1081 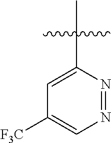 [SMILES: *c1cc(C(F)(F)F)cnn1] |
| R1-, R2-1082 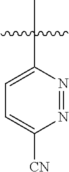 [SMILES: *c1ccc(C#N)nn1] | R1-, R2-1083 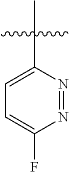 [SMILES: *c1ccc(F)nn1] | R1-, R2-1084 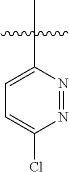 [SMILES: *c1ccc(Cl)nn1] | R1-, R2-1085 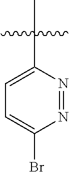 [SMILES: *c1ccc(Br)nn1] | R1-, R2-1086 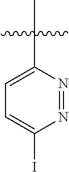 [SMILES: *c1ccc(I)nn1] | R1-, R2-1087 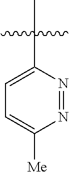 [SMILES: *c1ccc(C)nn1] |
| R1-, R2-1088 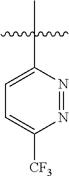 [SMILES: *c1ccc(C(F)(F)F)nn1] | R1-, R2-1089 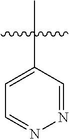 [SMILES: *c1ccnnc1] | R1-, R2-1090 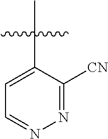 [SMILES: *c1ccnnc1C#N] | R1-, R2-1091 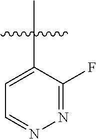 [SMILES: *c1ccnnc1F] | R1-, R2-1092 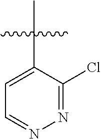 [SMILES: *c1ccnnc1Cl] | R1-, R2-1093 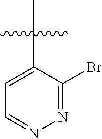 [SMILES: *c1ccnnc1Br] |
| R1-, R2-1094 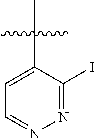 [SMILES: *c1ccnnc1I] | R1-, R2-1095 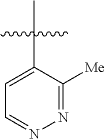 [SMILES: *c1ccnnc1C] | R1-, R2-1096 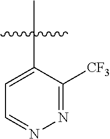 [SMILES: *c1ccnnc1C(F)(F)F] | R1-, R2-1097 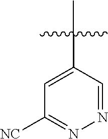 [SMILES: *c1cnnc(C#N)c1] | R1-, R2-1098 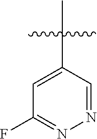 [SMILES: *c1cnnc(F)c1] | R1-, R2-1099 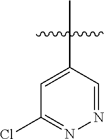 [SMILES: *c1cnnc(Cl)c1] |
| R1-, R2-1100 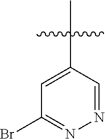 [SMILES: *c1cnnc(Br)c1] | R1-, R2-1101 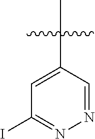 [SMILES: *c1cnnc(I)c1] | R1-, R2-1102 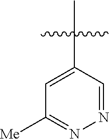 [SMILES: *c1cnnc(C)c1] | R1-, R2-1103 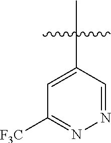 [SMILES: *c1cnnc(C(F)(F)F)c1] | R1-, R2-1104 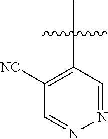 [SMILES: *c1cnncc1C#N] | R1-, R2-1105 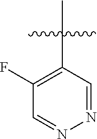 [SMILES: *c1cnncc1F] |
| R1-, R2-1106 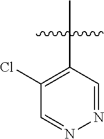 [SMILES: *c1cnncc1Cl] | R1-, R2-1107 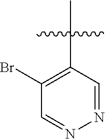 [SMILES: *c1cnncc1Br] | R1-, R2-1108 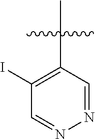 [SMILES: *c1cnncc1I] | R1-, R2-1109 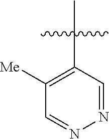 [SMILES: *c1cnncc1C] | R1-, R2-1110 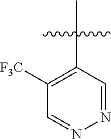 [SMILES: *c1cnncc1C(F)(F)F] | R1-, R2-1111 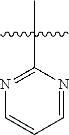 [SMILES: *c1ncccn1] |
| R1-, R2-1112 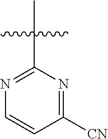 [SMILES: *c1nccc(C#N)n1] | R1-, R2-1113 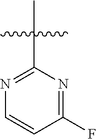 [SMILES: *c1nccc(F)n1] | R1-, R2-1114 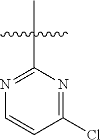 [SMILES: *c1nccc(Cl)n1] | R1-, R2-1115 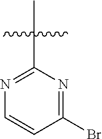 [SMILES: *c1nccc(Br)n1] | R1-, R2-1116 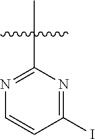 [SMILES: *c1nccc(I)n1] | R1-, R2-1117 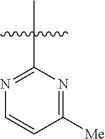 [SMILES: *c1nccc(C)n1] |
| R1-, R2-1118 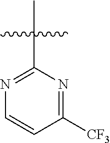 [SMILES: *c1nccc(C(F)(F)F)n1] | R1-, R2-1119 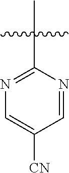 [SMILES: *c1ncc(C#N)cn1] | R1-, R2-1120 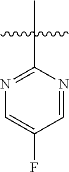 [SMILES: *c1ncc(F)cn1] | R1-, R2-1121 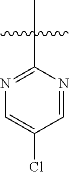 [SMILES: *c1ncc(Cl)cn1] | R1-, R2-1122 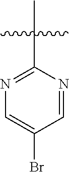 [SMILES: *c1ncc(Br)cn1] | R1-, R2-1123 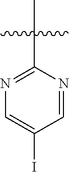 [SMILES: *c1ncc(I)cn1] |
| R1-, R2-1124 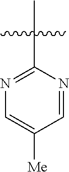 [SMILES: *c1ncc(C)cn1] | R1-, R2-1125 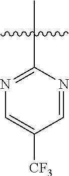 [SMILES: *c1ncc(C(F)(F)F)cn1] | R1-, R2-1126 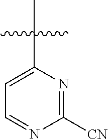 [SMILES: *c1ccnc(C#N)n1] | R1-, R2-1127 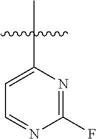 [SMILES: *c1ccnc(F)n1] | R1-, R2-1128 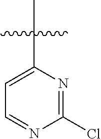 [SMILES: *c1ccnc(Cl)n1] | R1-, R2-1129 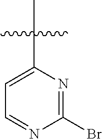 [SMILES: *c1ccnc(Br)n1] |
| R1-, R2-1130 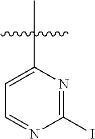 [SMILES: *c1ccnc(I)n1] | R1-, R2-1131 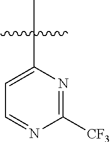 [SMILES: *c1ccnc(C(F)(F)F)n1] | R1-, R2-1132 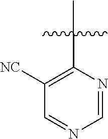 [SMILES: *c1ncncc1C#N] | R1-, R2-1133 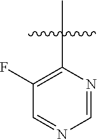 [SMILES: *c1ncncc1F] | R1-, R2-1134 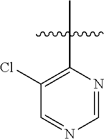 [SMILES: *c1ncncc1Cl] | R1-, R2-1135 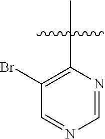 [SMILES: *c1ncncc1Br] |
| R1-, R2-1136 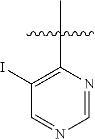 [SMILES: *c1ncncc1I] | R1-, R2-1137 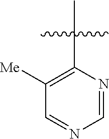 [SMILES: *c1ncncc1C] | R1-, R2-1138 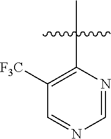 [SMILES: *c1ncncc1C(F)(F)F] | R1-, R2-1139 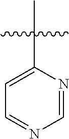 [SMILES: *c1ccncn1] | R1-, R2-1140 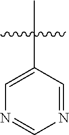 [SMILES: *c1cncnc1] | R1-, R2-1141 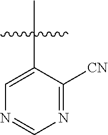 [SMILES: *c1cncnc1C#N] |
| R1-, R2-1142 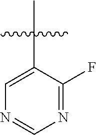 [SMILES: *c1cncnc1F] | R1-, R2-1143 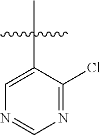 [SMILES: *c1cncnc1Cl] | R1-, R2-1144 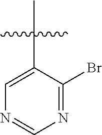 [SMILES: *c1cncnc1Br] | R1-, R2-1145 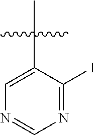 [SMILES: *c1cncnc1I] | R1-, R2-1146 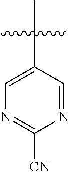 [SMILES: *c1cnc(C#N)nc1] | R1-, R2-1147 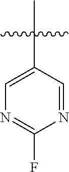 [SMILES: *c1cnc(F)nc1] |
| R1-, R2-1148 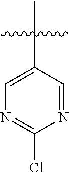 [SMILES: *c1cnc(Cl)nc1] | R1-, R2-1149 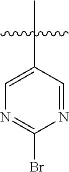 [SMILES: *c1cnc(Br)nc1] | R1-, R2-1150 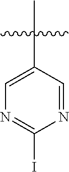 [SMILES: *c1cnc(I)nc1] | R1-, R2-1151 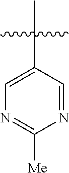 [SMILES: *c1cnc(C)nc1] | R1-, R2-1152 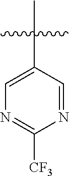 [SMILES: *c1cnc(C(F)(F)F)nc1] | R1-, R2-1153 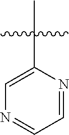 [SMILES: *c1cnccn1] |
| R1-, R2-1154 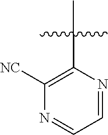 [SMILES: *c1nccnc1C#N] | R1-, R2-1155 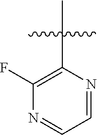 [SMILES: *c1nccnc1F] | R1-, R2-1156 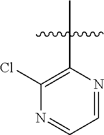 [SMILES: *c1nccnc1Cl] | R1-, R2-1157 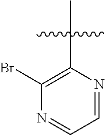 [SMILES: *c1nccnc1Br] | R1-, R2-1158 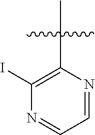 [SMILES: *c1nccnc1I] | R1-, R2-1159 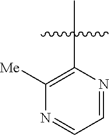 [SMILES: *c1nccnc1C] |
| R1-, R2-1160 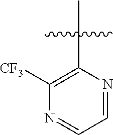 [SMILES: *c1nccnc1C(F)(F)F] | R1-, R2-1161 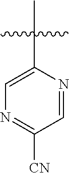 [SMILES: *c1cnc(C#N)cn1] | R1-, R2-1162 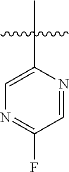 [SMILES: *c1cnc(F)cn1] | R1-, R2-1163 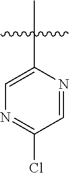 [SMILES: *c1cnc(Cl)cn1] | R1-, R2-1164 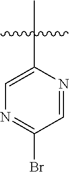 [SMILES: *c1cnc(Br)cn1] | R1-, R2-1165 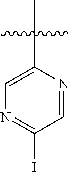 [SMILES: *c1cnc(I)cn1] |
| R1-, R2-1166 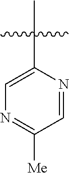 [SMILES: *c1cnc(C)cn1] | R1-, R2-1167 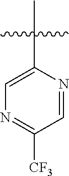 [SMILES: *c1cnc(C(F)(F)F)cn1] | R1-, R2-1168 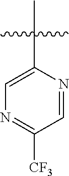 [SMILES: *c1cnc(C(F)(F)F)cn1] | R1-, R2-1169 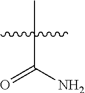 [SMILES: *C(N)=O] | R1-, R2-1170 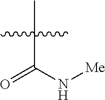 [SMILES: *C(=O)NC] | R1-, R2-1171 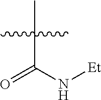 [SMILES: *C(=O)NCC] |
| R1-, R2-1172 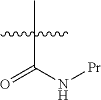 | R1-, R2-1173 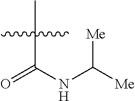 [SMILES: *C(=O)NC(C)C] | R1-, R2-1174 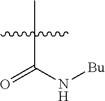 | R1-, R2-1175 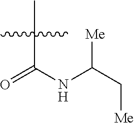 [SMILES: *C(=O)NC(C)CC] | R1-, R2-1176 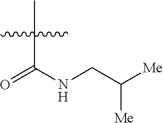 [SMILES: *C(=O)NCC(C)C] | R1-, R2-1177 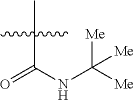 [SMILES: *C(=O)NC(C)(C)C] |
| R1-, R2-1178 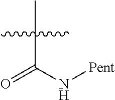 | R1-, R2-1179 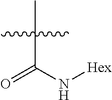 | R1-, R2-1180 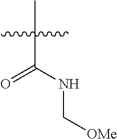 [SMILES: *C(=O)NCOC] | R1-, R2-1181 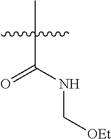 [SMILES: *C(=O)NCOCC] | R1-, R2-1182 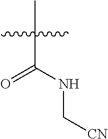 [SMILES: *C(=O)NCC#N] | R1-, R2-1183 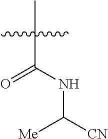 [SMILES: *C(=O)NC(C)C#N] |
| R1-, R2-1184 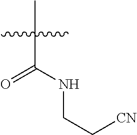 [SMILES: *C(=O)NCCC#N] | R1-, R2-1185 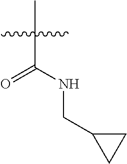 [SMILES: *C(=O)NCC1CC1] | R1-, R2-1186 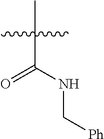 [SMILES: *C(=O)NCc1ccccc1] | R1-, R2-1187 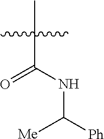 [SMILES: *C(=O)NC(C)c1ccccc1] | R1-, R2-1188 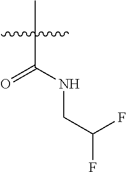 [SMILES: *C(=O)NCC(F)F] | R1-, R2-1189 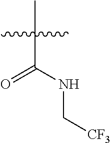 [SMILES: *C(=O)NCC(F)(F)F] |
| R1-, R2-1190 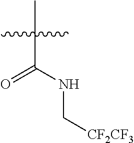 | R1-, R2-1191 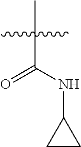 [SMILES: *C(=O)NC1CC1] | R1-, R2-1192 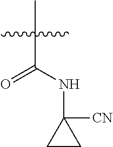 [SMILES: *C(=O)NC1(C#N)CC1] | R1-, R2-1193 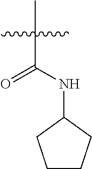 [SMILES: *C(=O)NC1CCCC1] | R1-, R2-1194 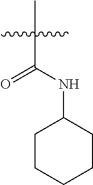 [SMILES: *C(=O)NC1CCCCC1] | R1-, R2-1195 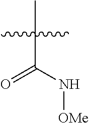 [SMILES: *C(=O)NOC] |
| R1-, R2-1196 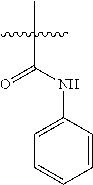 [SMILES: *C(=O)Nc1ccccc1] | R1-, R2-1197 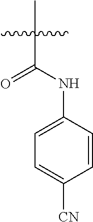 [SMILES: *C(=O)Nc1ccc(C#N)cc1] | R1-, R2-1198 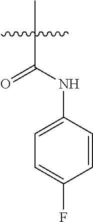 [SMILES: *C(=O)Nc1ccc(F)cc1] | R1-, R2-1199 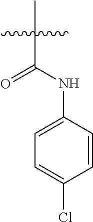 [SMILES: *C(=O)Nc1ccc(Cl)cc1] | R1-, R2-1200 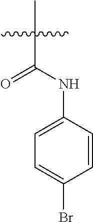 [SMILES: *C(=O)Nc1ccc(Br)cc1] | R1-, R2-1201 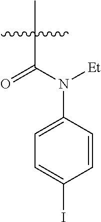 [SMILES: *C(=O)N(CC)c1ccc(I)cc1] |
| R1-, R2-1202 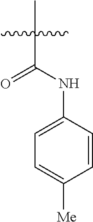 [SMILES: *C(=O)Nc1ccc(C)cc1] | R1-, R2-1203 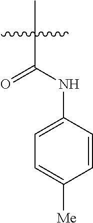 [SMILES: *C(=O)Nc1ccc(C)cc1] | R1-, R2-1204 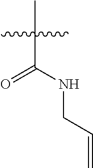 [SMILES: *C(=O)NCC=C] | R1-, R2-1205 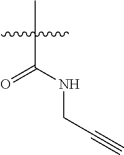 [SMILES: *C(=O)NCC#C] | R1-, R2-1206 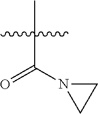 [SMILES: *C(=O)N1CC1] | R1-, R2-1207 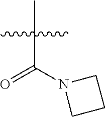 [SMILES: *C(=O)N1CCC1] |
| R1-, R2-1208 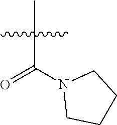 [SMILES: *C(=O)N1CCCC1] | R1-, R2-1209 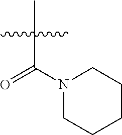 [SMILES: *C(=O)N1CCCCC1] | R1-, R2-1210 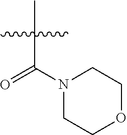 [SMILES: *C(=O)N1CCOCC1] | R1-, R2-1211 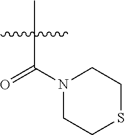 [SMILES: *C(=O)N1CCSCC1] | R1-, R2-1212 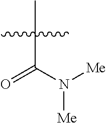 [SMILES: *C(=O)N(C)C] | R1-, R2-1213 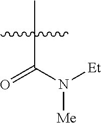 [SMILES: *C(=O)N(C)CC] |
| R1-, R2-1214 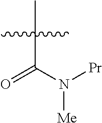 | R1-, R2-1215 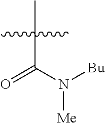 | R1-, R2-1216 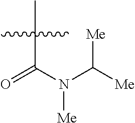 [SMILES: *C(=O)N(C)C(C)C] | R1-, R2-1217 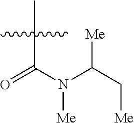 [SMILES: *C(=O)N(C)C(C)CC] | R1-, R2-1218 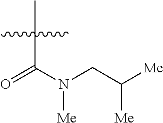 [SMILES: *C(=O)N(C)CC(C)C] | R1-, R2-1219 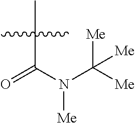 [SMILES: *C(=O)N(C)C(C)(C)C] |
| R1-, R2-1220 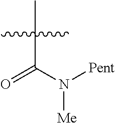 | R1-, R2-1221 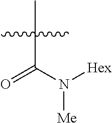 | R1-, R2-1222 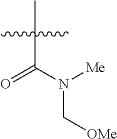 [SMILES: *C(=O)N(C)COC] | R1-, R2-1223 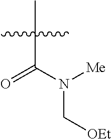 [SMILES: *C(=O)N(C)COCC] | R1-, R2-1224 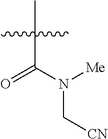 [SMILES: *C(=O)N(C)CC#N] | R1-, R2-1225 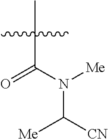 [SMILES: *C(=O)N(C)C(C)C#N] |
| R1-, R2-1226 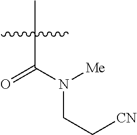 [SMILES: *C(=O)N(C)CCC#N] | R1-, R2-1227 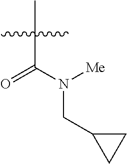 [SMILES: *C(=O)N(C)CC1CC1] | R1-, R2-1228 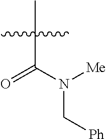 [SMILES: *C(=O)N(C)Cc1ccccc1] | R1-, R2-1229 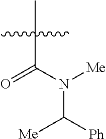 [SMILES: *C(=O)N(C)C(C)c1ccccc1] | R1-, R2-1230 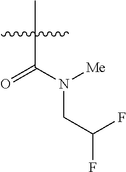 [SMILES: *C(=O)N(C)CC(F)F] | R1-, R2-1231 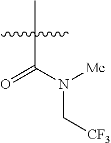 [SMILES: *C(=O)N(C)CC(F)(F)F] |
| R1-, R2-1232 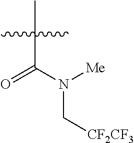 | R1-, R2-1233 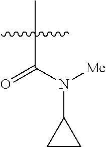 [SMILES: *C(=O)N(C)C1CC1] | R1-, R2-1234 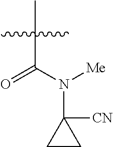 [SMILES: *C(=O)N(C)C1(C#N)CC1] | R1-, R2-1235 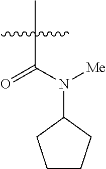 [SMILES: *C(=O)N(C)C1CCCC1] | R1-, R2-1236 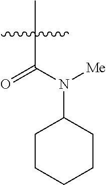 [SMILES: *C(=O)N(C)C1CCCCC1] | R1-, R2-1237 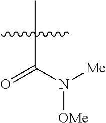 [SMILES: *C(=O)N(C)OC] |
| R1-, R2-1238 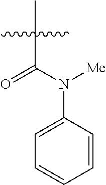 [SMILES: *C(=O)N(C)c1ccccc1] | R1-, R2-1239 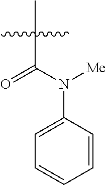 [SMILES: *C(=O)N(C)c1ccccc1] | R1-, R2-1240 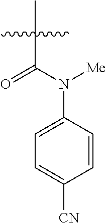 [SMILES: *C(=O)N(C)c1ccc(C#N)cc1] | R1-, R2-1241 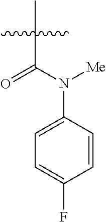 [SMILES: *C(=O)N(C)c1ccc(F)cc1] | R1-, R2-1242 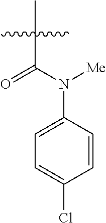 [SMILES: *C(=O)N(C)c1ccc(Cl)cc1] | R1-, R2-1243 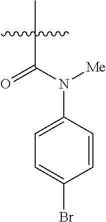 [SMILES: *C(=O)N(C)c1ccc(Br)cc1] |
| R1-, R2-1244 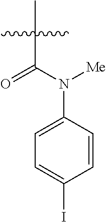 [SMILES: *C(=O)N(C)c1ccc(I)cc1] | R1-, R2-1245 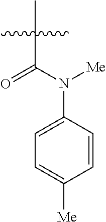 [SMILES: *C(=O)N(C)c1ccc(C)cc1] | R1-, R2-1246 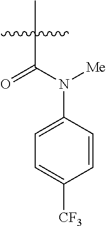 [SMILES: *C(=O)N(C)c1ccc(C(F)(F)F)cc1] | R1-, R2-1247 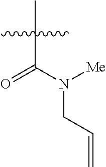 [SMILES: *C(=O)N(C)CC=C] | R1-, R2-1248 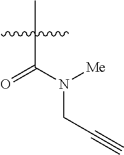 [SMILES: *C(=O)N(C)CC#C] | R1-, R2-1249 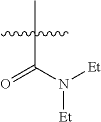 [SMILES: *C(=O)N(CC)CC] |
| R1-, R2-1250 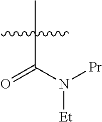 | R1-, R2-1251 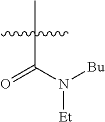 | R1-, R2-1252 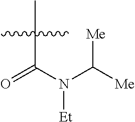 [SMILES: *C(=O)N(CC)C(C)C] | R1-, R2-1253 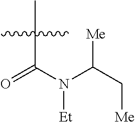 [SMILES: *C(=O)N(CC)C(C)CC] | R1-, R2-1254 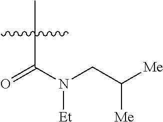 [SMILES: *C(=O)N(CC)CC(C)C] | R1-, R2-1255 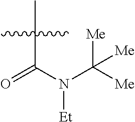 [SMILES: *C(=O)N(CC)C(C)(C)C] |
| R1-, R2-1256 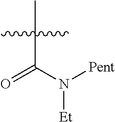 | R1-, R2-1257 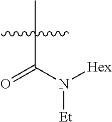 | R1-, R2-1258 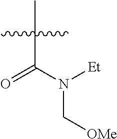 [SMILES: *C(=O)N(CC)COC] | R1-, R2-1259 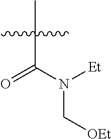 [SMILES: *C(=O)N(CC)COCC] | R1-, R2-1260 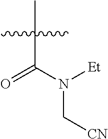 [SMILES: *C(=O)N(CC)CC#N] | R1-, R2-1261 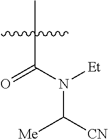 [SMILES: *C(=O)N(CC)C(C)C#N] |
| R1-, R2-1262 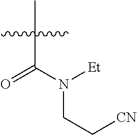 [SMILES: *C(=O)N(CC)CCC#N] | R1-, R2-1263 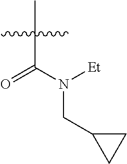 [SMILES: *C(=O)N(CC)CC1CC1] | R1-, R2-1264 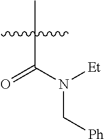 [SMILES: *C(=O)N(CC)Cc1ccccc1] | R1-, R2-1265 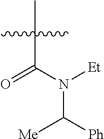 [SMILES: *C(=O)N(CC)C(C)c1ccccc1] | R1-, R2-1266 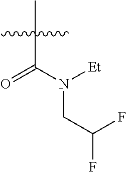 [SMILES: *C(=O)N(CC)CC(F)F] | R1-, R2-1267 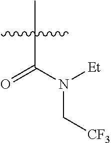 [SMILES: *C(=O)N(CC)CC(F)(F)F] |
| R1-, R2-1268 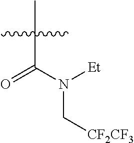 | R1-, R2-1269 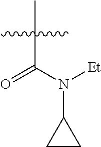 [SMILES: *C(=O)N(CC)C1CC1] | R1-, R2-1270 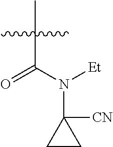 [SMILES: *C(=O)N(CC)C1(C#N)CC1] | R1-, R2-1271 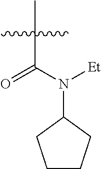 [SMILES: *C(=O)N(CC)C1CCCC1] | R1-, R2-1272 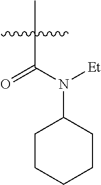 [SMILES: *C(=O)N(CC)C1CCCCC1] | R1-, R2-1273 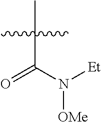 [SMILES: *C(=O)N(CC)OC] |
| R1-, R2-1274 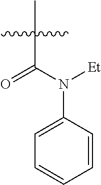 [SMILES: *C(=O)N(CC)c1ccccc1] | R1-, R2-1275 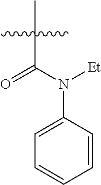 [SMILES: *C(=O)N(CC)c1ccccc1] | R1-, R2-1276 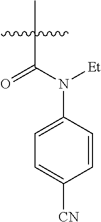 [SMILES: *C(=O)N(CC)c1ccc(C#N)cc1] | R1-, R2-1277 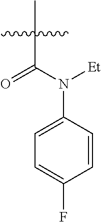 [SMILES: *C(=O)N(CC)c1ccc(F)cc1] | R1-, R2-1278 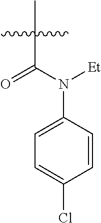 [SMILES: *C(=O)N(CC)c1ccc(Cl)cc1] | R1-, R2-1279 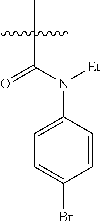 [SMILES: *C(=O)N(CC)c1ccc(Br)cc1] |
| R1-, R2-1280 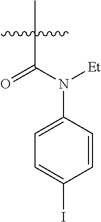 [SMILES: *C(=O)N(CC)c1ccc(I)cc1] | R1-, R2-1281 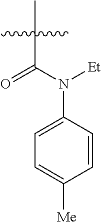 [SMILES: *C(=O)N(CC)c1ccc(C)cc1] | R1-, R2-1282 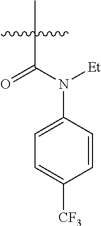 [SMILES: *C(=O)N(CC)c1ccc(C(F)(F)F)cc1] | R1-, R2-1283 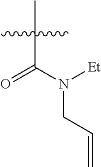 [SMILES: *C(=O)N(CC)CC=C] | R1-, R2-1284 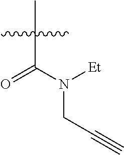 [SMILES: *C(=O)N(CC)CC#C] | R1-, R2-1285 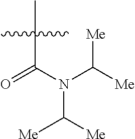 [SMILES: *C(=O)N(C(C)C)C(C)C] |
| R1-, R2-1286 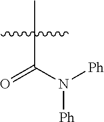 [SMILES: *C(=O)N(c1ccccc1)c1ccccc1] | R1-, R2-1287 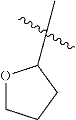 [SMILES: *C1CCCO1] | R1-, R2-1288 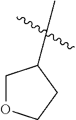 [SMILES: *C1CCOC1] | R1-, R2-1289 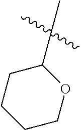 [SMILES: *C1CCCCO1] | R1-, R2-1290 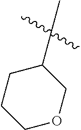 [SMILES: *C1CCCOC1] | R1-, R2-1291 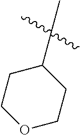 [SMILES: *C1CCOCC1] |
| R1-, R2-1292 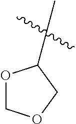 [SMILES: *C1COCO1] | R1-, R2-1293 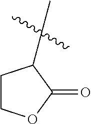 [SMILES: *C1CCOC1=O] | R1-, R2-1294 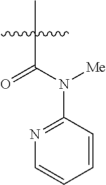 [SMILES: *C(=O)N(C)c1ccccn1] | R1-, R2-1295 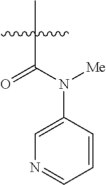 [SMILES: *C(=O)N(C)c1cccnc1] | R1-, R2-1296 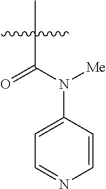 [SMILES: *C(=O)N(C)c1ccncc1] | R1-, R2-1297 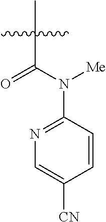 [SMILES: *C(=O)N(C)c1ccc(C#N)cn1] |
| R1-, R2-1298 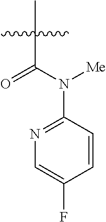 [SMILES: *C(=O)N(C)c1ccc(F)cn1] | R1-, R2-1299 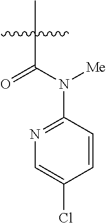 [SMILES: *C(=O)N(C)c1ccc(Cl)cn1] | R1-, R2-1300 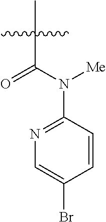 [SMILES: *C(=O)N(C)c1ccc(Br)cn1] | R1-, R2-1301 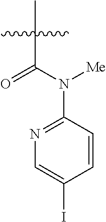 [SMILES: *C(=O)N(C)c1ccc(I)cn1] | R1-, R2-1302 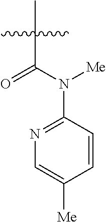 [SMILES: *C(=O)N(C)c1ccc(C)cn1] | R1-, R2-1303 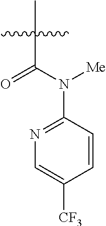 [SMILES: *C(=O)N(C)c1ccc(C(F)(F)F)cn1] |
| R1-, R2-1304 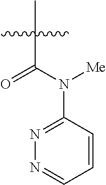 [SMILES: *C(=O)N(C)c1cccnn1] | R1-, R2-1305 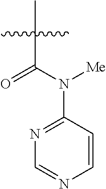 [SMILES: *C(=O)N(C)c1ccncn1] | R1-, R2-1306 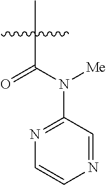 [SMILES: *C(=O)N(C)c1cnccn1] | R1-, R2-1307 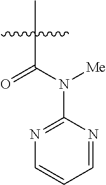 [SMILES: *C(=O)N(C)c1ncccn1] | R1-, R2-1308 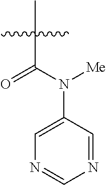 [SMILES: *C(=O)N(C)c1cncnc1] | R1-, R2-1309 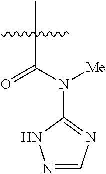 [SMILES: *C(=O)N(C)c1ncn[nH]1] |
| R1-, R2-1310 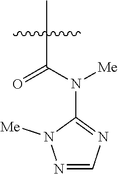 [SMILES: *C(=O)N(C)c1ncnn1C] | R1-, R2-1311 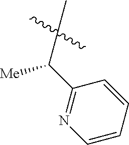 [SMILES: *[C@@H](C)c1ccccn1] | R1-, R2-1312 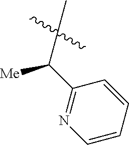 [SMILES: *[C@H](C)c1ccccn1] | R1-, R2-1313 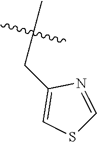 [SMILES: *Cc1cscn1] | R1-, R2-1314 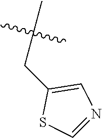 [SMILES: *Cc1cncs1] | R1-, R2-1315 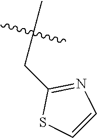 [SMILES: *Cc1nccs1] |

TABLE 4

| | R3 | | | R3 | | | R3 | |
|---|---|---|---|---|---|---|---|---|
| | substituent | | | substituent | | | substituent | |
| No. | example | R4 substituent example | No. | example | R4 substituent example | No. | example | R4 substituent example |
| Z-1 | —H | —H | Z-2 | —Me | —H | Z-3 | —Et | —H |
| Z-4 | —H | —Me | Z-5 | —Me | —Me | Z-6 | —Et | —Me |
| Z-7 | —H | —Et | Z-8 | —Me | —Et | Z-9 | —Et | —Et |
| Z-10 | —H | —Pr | Z-11 | —Me | —Pr | Z-12 | —Et | —Pr |
| Z-13 | —H | —i-Pr | Z-14 | —Me | —i-Pr | Z-15 | —Et | —i-Pr |
| Z-16 | —H | —Bu | Z-17 | —Me | —Bu | Z-18 | —Et | —Bu |
| Z-19 | —H | —sec-Bu | Z-20 | —Me | —sec-Bu | Z-21 | —Et | —sec-Bu |
| Z-22 | —H | —I-Bu | Z-23 | —Me | —I-Bu | Z-24 | —Et | —I-Bu |
| Z-25 | —H | —t-Bu | Z-26 | —Me | —t-Bu | Z-27 | —Et | —t-Bu |
| Z-28 | —H | —Pent | Z-29 | —Me | —Pent | Z-30 | —Et | —Pent |
| Z-31 | —H | —Hex | Z-32 | —Me | —Hex | Z-33 | —Et | —Hex |
| Z-34 | —H | —CH(CH₃)CH₂CH₂CH₃ | Z-35 | —Me | —CH(CH₃)CH₂CH₂CH₃ | Z-36 | —Et | —CH(CH₃)CH₂CH₂CH₃ |
| Z-37 | —H | —CH(CH₃)CH(CH₃)₂ | Z-38 | —Me | —CH(CH₃)CH(CH₃)₂ | Z-39 | —Et | —CH(CH₃)CH(CH₃)₂ |
| Z-40 | —H | —C(CH₃)₂CH₂CH₃ | Z-41 | —Me | —C(CH₃)₂CH₂CH₃ | Z-42 | —Et | —C(CH₃)₂CH₂CH₃ |
| Z-43 | —H | —CH(CH₂CH₃)₂ | Z-44 | —Me | —CH(CH₂CH₃)₂ | Z-45 | —Et | —CH(CH₂CH₃)₂ |
| Z-46 | —H | —CH₂CH₂CH(CH₃)₂ | Z-47 | —Me | —CH₂CH₂CH(CH₃)₂ | Z-48 | —Et | —CH₂CH₂CH(CH₃)₂ |
| Z-49 | —H | —CH₂CH₂CH₂CH(CH₃)₂ | Z-50 | —Me | —CH₂CH₂CH₂CH(CH₃)₂ | Z-51 | —Et | —CH₂CH₂CH₂CH(CH₃)₂ |
| Z-52 | —H | —CH₂CH₂CH(CH₃)CH₂CH₃ | Z-53 | —Me | —CH₂CH₂CH(CH₃)CH₂CH₃ | Z-54 | —Et | —CH₂CH₂CH(CH₃)CH₂CH₃ |
| Z-55 | —H | —CH₂CH(CH₃)CH₂CH₂CH₃ | Z-56 | —Me | —CH₂CH(CH₃)CH₂CH₂CH₃ | Z-57 | —Et | —CH₂CH(CH₃)CH₂CH₂CH₃ |
| Z-58 | —H | —CH(CH₃)CH₂CH₂CH₂CH₃ | Z-59 | —Me | —CH(CH₃)CH₂CH₂CH₂CH₃ | Z-60 | —Et | —CH(CH₃)CH₂CH₂CH₂CH₃ |
| Z-61 | —H | —CH(CH3)CH₂CH(CH₃)₂ | Z-62 | —Me | —CH(CH3)CH₂CH(CH₃)₂ | Z-63 | —Et | —CH(CH3)CH₂CH(CH₃)₂ |
| Z-64 | —H | —CH(CH₃)CH(CH₃)CH₂CH₃ | Z-65 | —Me | —CH(CH₃)CH(CH₃)CH₂CH₃ | Z-66 | —Et | —CH(CH₃)CH(CH₃)CH₂CH₃ |
| Z-67 | —H | —C(CH₃)₂CH₂CH₂CH₃ | Z-68 | —Me | —C(CH₃)₂CH₂CH₂CH₃ | Z-69 | —Et | —C(CH₃)₂CH₂CH₂CH₃ |
| Z-70 | —H | —CH(CH₃)C(CH₃)₃ | Z-71 | —Me | —CH(CH₃)C(CH₃)₃ | Z-72 | —Et | —CH(CH₃)C(CH₃)₃ |
| Z-73 | —H | —C(CH₃)₂CH(CH₃)₂ | Z-74 | —Me | —C(CH₃)₂CH(CH₃)₂ | Z-75 | —Et | —C(CH₃)₂CH(CH₃)₂ |
| Z-76 | —H | —CH₂CH₂C(CH₃)₃ | Z-77 | —Me | —CH₂CH₂C(CH₃)₃ | Z-78 | —Et | —CH₂CH₂C(CH₃)₃ |
| Z-79 | —H | —CH₂CH(CH₃)CH(CH₃)₂ | Z-80 | —Me | —CH₂CH(CH₃)CH(CH₃)₂ | Z-81 | —Et | —CH₂CH(CH₃)CH(CH₃)₂ |
| Z-82 | —H | —CH₂C(CH₃)₂CH₂CH₃ | Z-83 | —Me | —CH₂C(CH₃)₂CH₂CH₃ | Z-84 | —Et | —CH₂C(CH₃)₂CH₂CH₃ |
| Z-85 | —H | —CFH₂ | Z-86 | —Me | —CFH₂ | Z-87 | —Et | —CFH₂ |
| Z-88 | —H | —CF₂H | Z-89 | —Me | —CF₂H | Z-90 | —Et | —CF₂H |
| Z-91 | —H | —CF₃ | Z-92 | —Me | —CF₃ | Z-93 | —Et | —CF₃ |
| Z-94 | —H | —CH₂Cl | Z-95 | —Me | —CH₂Cl | Z-96 | —Et | —CH₂Cl |
| Z-97 | —H | —CHCl₂ | Z-98 | —Me | —CHCl₂ | Z-99 | —Et | —CHCl₂ |
| Z-100 | —H | —CCl₃ | Z-101 | —Me | —CCl₃ | Z-102 | —Et | —CCl₃ |
| Z-103 | —H | —CF₂Cl | Z-104 | —Me | —CF₂Cl | Z-105 | —Et | —CF₂Cl |
| Z-106 | —H | —CCl₂F | Z-107 | —Me | —CCl₂F | Z-108 | —Et | —CCl₂F |
| Z-109 | —H | —CH₂Br | Z-110 | —Me | —CH₂Br | Z-111 | —Et | —CH₂Br |
| Z-112 | —H | —CHBr₂ | Z-113 | —Me | —CHBr₂ | Z-114 | —Et | —CHBr₂ |
| Z-115 | —H | —CBr₃ | Z-116 | —Me | —CBr₃ | Z-117 | —Et | —CBr₃ |
| Z-118 | —H | —CH₂l | Z-119 | —Me | —CH₂l | Z-120 | —Et | —CH₂l |
| Z-121 | —H | —CHl₂ | Z-122 | —Me | —CHl₂ | Z-123 | —Et | —CHl₂ |
| Z-124 | —H | —CH₂CF₂H | Z-125 | —Me | —CH₂CF₂H | Z-126 | —Et | —CH₂CF₂H |
| Z-127 | —H | —CH₂CF₃ | Z-128 | —Me | —CH₂CF₃ | Z-129 | —Et | —CH₂CF₃ |
| Z-130 | —H | —CF₂CH₃ | Z-131 | —Me | —CF₂CH₃ | Z-132 | —Et | —CF₂CH₃ |
| Z-133 | —H | —CH₂CH₂CF₂H | Z-134 | —Me | —CH₂CH₂CF₂H | Z-135 | —Et | —CH₂CH₂CF₂H |
| Z-136 | —H | —CH₂CH₂CF₃ | Z-137 | —Me | —CH₂CH₂CF₃ | Z-138 | —Et | —CH₂CH₂CF₃ |
| Z-139 | —H | —CH₂CH₂CH₂CF₂H | Z-140 | —Me | —CH₂CH₂CH₂CF₂H | Z-141 | —Et | —CH₂CH₂CH₂CF₂H |
| Z-142 | —H | —CH₂CH₂CH₂CF₃ | Z-143 | —Me | —CH₂CH₂CH₂CF₃ | Z-144 | —Et | —CH₂CH₂CH₂CF₃ |
| Z-145 | —H | —CF₂CF₂H | Z-146 | —Me | —CF₂CF₂H | Z-147 | —Et | —CF₂CF₂H |
| Z-148 | —H | —CF₂CF₂Cl | Z-149 | —Me | —CF₂CF₂Cl | Z-150 | —Et | —CF₂CF₂Cl |
| Z-151 | —H | —CF₂CF₃ | Z-152 | —Me | —CF₂CF₃ | Z-153 | —Et | —CF₂CF₃ |
| Z-154 | —H | —CFHCF₃ | Z-155 | —Me | —CFHCF₃ | Z-156 | —Et | —CFHCF₃ |
| Z-157 | —H | —CH₂CF₂CF₂H | Z-158 | —Me | —CH₂CF₂CF₂H | Z-159 | —Et | —CH₂CF₂CF₂H |
| Z-160 | —H | —CH₂CF₂CF₃ | Z-161 | —Me | —CH₂CF₂CF₃ | Z-162 | —Et | —CH₂CF₂CF₃ |
| Z-163 | —H | —CF₂CF₂CF₃ | Z-164 | —Me | —CF₂CF₂CF₃ | Z-165 | —Et | —CF₂CF₂CF₃ |
| Z-166 | —H | —CH₂CF₂CF₂CF₃ | Z-167 | —Me | —CH₂CF₂CF₂CF₃ | Z-168 | —Et | —CH₂CF₂CF₂CF₃ |
| Z-169 | —H | —CF₂CF₂CF₂CF₃ | Z-170 | —Me | —CF₂CF₂CF₂CF₃ | Z-171 | —Et | —CF₂CF₂CF₂CF₃ |
| Z-172 | —H | —CH₂CF₂CF₂CF₂CF₃ | Z-173 | —Me | —CH₂CF₂CF₂CF₂CF₃ | Z-174 | —Et | —CH₂CF₂CF₂CF₂CF₃ |
| Z-175 | —H | c-Pr | Z-176 | —Me | c-Pr | Z-177 | —Et | c-Pr |
| Z-178 | —H | c-Bu | Z-179 | —Me | c-Bu | Z-180 | —Et | c-Bu |
| Z-181 | —H | c-Pent | Z-182 | —Me | c-Pent | Z-183 | —Et | c-Pent |
| Z-184 | —H | c-Hex | Z-185 | —Me | c-Hex | Z-186 | —Et | c-Hex |
| Z-187 | —H | c-Hept | Z-188 | —Me | c-Hept | Z-189 | —Et | c-Hept |
| Z-190 | —H | c-Oct | Z-191 | —Me | c-Oct | Z-192 | —Et | c-Oct |
| Z-193 | —H | —CH═CH₂ | Z-194 | —Me | —CH═CH₂ | Z-195 | —Et | —CH═CH₂ |
| Z-196 | —H | —CH₂CH═CH₂ | Z-197 | —Me | —CH₂CH═CH₂ | Z-198 | —Et | —CH₂CH═CH₂ |
| Z-199 | —H | —CH═CHCH₃ | Z-200 | —Me | —CH═CHCH₃ | Z-201 | —Et | —CH═CHCH₃ |
| Z-202 | —H | —CH₂C(CH₃)═CH₂ | Z-203 | —Me | —CH₂C(CH₃)═CH₂ | Z-204 | —Et | —CH₂C(CH₃)═CH₂ |
| Z-205 | —H | —CH₂CH₂CH═CH₂ | Z-206 | —Me | —CH₂CH₂CH═CH₂ | Z-207 | —Et | —CH₂CH₂CH═CH₂ |
| Z-208 | —H | —CH₂CH═CHCH₃ | Z-209 | —Me | —CH₂CH═CHCH₃ | Z-210 | —Et | —CH₂CH═CHCH₃ |
| Z-211 | —H | —CH═CHCH₂CH₃ | Z-212 | —Me | —CH═CHCH₂CH₃ | Z-213 | —Et | —CH═CHCH₂CH₃ |
| Z-214 | —H | —CH₂CH═C(CH₃)₂ | Z-215 | —Me | —CH₂CH═C(CH₃)₂ | Z-216 | —Et | —CH₂CH═C(CH₃)₂ |
| Z-217 | —H | —CH₂CH₂CH═C(CH₃)₂ | Z-218 | —Me | —CH₂CH₂CH═C(CH₃)₂ | Z-219 | —Et | —CH₂CH₂CH═C(CH₃)₂ |
| Z-220 | —H | —CH═CFH | Z-221 | —Me | —CH═CFH | Z-222 | —Et | —CH═CFH |
| Z-223 | —H | —CH═CF₂ | Z-224 | —Me | —CH═CF₂ | Z-225 | —Et | —CH═CF₂ |
| Z-226 | —H | —CH═CCl₂ | Z-227 | —Me | —CH═CCl₂ | Z-228 | —Et | —CH═CCl₂ |
| Z-229 | —H | —CH₂CH═CFH | Z-230 | —Me | —CH₂CH═CFH | Z-231 | —Et | —CH₂CH═CFH |
| Z-232 | —H | —CH₂CH═CF₂ | Z-233 | —Me | —CH₂CH═CF₂ | Z-234 | —Et | —CH₂CH═CF₂ |
| Z-235 | —H | —CH₂CH═CCl₂ | Z-236 | —Me | —CH₂CH═CCl₂ | Z-237 | —Et | —CH₂CH═CCl₂ |
| Z-238 | —H | —CH₂CH₂CH═CF₂ | Z-239 | —Me | —CH₂CH₂CH═CF₂ | Z-240 | —Et | —CH₂CH₂CH═CF₂ |
| Z-241 | —H | —CH₂CH₂CH₂CH═CF₂ | Z-242 | —Me | —CH₂CH₂CH₂CH═CF₂ | Z-243 | —Et | —CH₂CH₂CH₂CH═CF₂ |
| Z-244 | —H | —CH₂CH₂CH₂CH₂CH═CF₂ | Z-245 | —Me | —CH₂CH₂CH₂CH₂CH═CF₂ | Z-246 | —Et | —CH₂CH₂CH₂CH₂CH═CF₂ |
| Z-247 | —H | —C≡CH | Z-248 | —Me | —C≡CH | Z-249 | —Et | —C≡CH |
| Z-250 | —H | —C≡CCH₃ | Z-251 | —Me | —C≡CCH₃ | Z-252 | —Et | —C≡CCH₃ |
| Z-253 | —H | —CH₂C≡CH | Z-254 | —Me | —CH₂C≡CH | Z-255 | —Et | —CH₂C≡CH |
| Z-256 | —H | —C≡CCH₂CH₃ | Z-257 | —Me | —C≡CCH₂CH₃ | Z-258 | —Et | —C≡CCH₂CH₃ |
| Z-259 | —H | —CH₂C≡CCH₃ | Z-260 | —Me | —CH₂C≡CCH₃ | Z-261 | —Et | —CH₂C≡CCH₃ |
| Z-262 | —H | —CH₂CH₂C≡CH | Z-263 | —Me | —CH₂CH₂C≡CH | Z-264 | —Et | —CH₂CH₂C≡CH |
| Z-265 | —H | —C≡CCH₂CH₂CH₃ | Z-266 | —Me | —C≡CCH₂CH₂CH₃ | Z-267 | —Et | —C≡CCH₂CH₂CH₃ |
| Z-268 | —H | —CH₂C≡CCH₂CH₃ | Z-269 | —Me | —CH₂C≡CCH₂CH₃ | Z-270 | —Et | —CH₂C≡CCH₂CH₃ |
| Z-271 | —H | —C(CH₃)₂C≡CH | Z-272 | —Me | —C(CH₃)₂C≡CH | Z-273 | —Et | —C(CH₃)₂C≡CH |
| Z-274 | —H | —C≡CF | Z-275 | —Me | —C≡CF | Z-276 | —Et | —C≡CF |
| Z-277 | —H | —C≡CCF₂H | Z-278 | —Me | —C≡CCF₂H | Z-279 | —Et | —C≡CCF₂H |
| Z-280 | —H | —C≡CCF₃ | Z-281 | —Me | —C≡CCF₃ | Z-282 | —Et | —C≡CCF₃ |
| Z-283 | —H | —C≡CCH₂CF₂H | Z-284 | —Me | —C≡CCH₂CF₂H | Z-285 | —Et | —C≡CCH₂CF₂H |
| Z-286 | —H | —C≡CCH₂CF₃ | Z-287 | —Me | —C≡CCH₂CF₃ | Z-288 | —Et | —C≡CCH₂CF₃ |
| Z-289 | —H | —CH₂C≡CHCF₂H | Z-290 | —Me | —CH₂C≡CHCF₂H | Z-291 | —Et | —CH₂C≡CHCF₂H |
| Z-292 | —H | —CH₂C≡CCF₃ | Z-293 | —Me | —CH₂C≡CCF₃ | Z-294 | —Et | —CH₂C≡CCF₃ |
| Z-295 | —H | —C(═O)NH₂ | Z-296 | —Me | —C(═O)NH₂ | Z-297 | —Et | —C(═O)NH₂ |
| Z-298 | —H | —C(═O)NHMe | Z-299 | —Me | —C(═O)NHMe | Z-300 | —Et | —C(═O)NHMe |
| Z-301 | —H | —C(═O)NHEt | Z-302 | —Me | —C(═O)NHEt | Z-303 | —Et | —C(═O)NHEt |
| Z-304 | —H | —C(═O)NHPr | Z-305 | —Me | —C(═O)NHPr | Z-306 | —Et | —C(═O)NHPr |
| Z-307 | —H | —C(═O)NH—i-Pr | Z-308 | —Me | —C(═O)NH—i-Pr | Z-309 | —Et | —C(═O)NH—i-Pr |
| Z-310 | —H | —C(═O)NHBu | Z-311 | —Me | —C(═O)NHBu | Z-312 | —Et | —C(═O)NHBu |
| Z-313 | —H | —C(═O)NH—sec-Bu | Z-314 | —Me | —C(═O)NH—sec-Bu | Z-315 | —Et | —C(═O)NH—sec-Bu |
| Z-316 | —H | —C(═O)NH—i-Bu | Z-317 | —Me | —C(═O)NH—i-Bu | Z-318 | —Et | —C(═O)NH—i-Bu |
| Z-319 | —H | —C(═O)NH—t-Bu | Z-320 | —Me | —C(═O)NH—t-Bu | Z-321 | —Et | —C(═O)NH—t-Bu |
| Z-322 | —H | —C(═O)NHPent | Z-323 | —Me | —C(═O)NHPent | Z-324 | —Et | —C(═O)NHPent |
| Z-325 | —H | —C(═O)NHHex | Z-326 | —Me | —C(═O)NHHex | Z-327 | —Et | —C(═O)NHHex |
| Z-328 | —H | —C(═O)NHCH₂C≡N | Z-329 | —Me | —C(═O)NHCH₂C≡N | Z-330 | —Et | —C(═O)NHCH₂C≡N |
| Z-331 | —H | —C(═O)NHCH₂—c-Pr | Z-332 | —Me | —C(═O)NHCH₂—c-Pr | Z-333 | —Et | —C(═O)NHCH₂—c-Pr |
| Z-334 | —H | —C(═O)NHCH₂OMe | Z-335 | —Me | —C(═O)NHCH₂OMe | Z-336 | —Et | —C(═O)NHCH₂OMe |
| Z-337 | —H | —C(═O)NHCH₂CH₂OMe | Z-338 | —Me | —C(═O)NHCH₂CH₂OMe | Z-339 | —Et | —C(═O)NHCH₂CH₂OMe |
| Z-340 | —H | —C(═O)NHCFH₂ | Z-341 | —Me | —C(═O)NHCFH₂ | Z-342 | —Et | —C(═O)NHCFH₂ |
| Z-343 | —H | —C(═O)NHCF₂H | Z-344 | —Me | —C(═O)NHCF₂H | Z-345 | —Et | —C(═O)NHCF₂H |
| Z-346 | —H | —C(═O)NHCF₃ | Z-347 | —Me | —C(═O)NHCF₃ | Z-348 | —Et | —C(═O)NHCF₃ |
| Z-349 | —H | —C(═O)NHCH₂Cl | Z-350 | —Me | —C(═O)NHCH₂Cl | Z-351 | —Et | —C(═O)NHCH₂Cl |
| Z-352 | —H | —C(═O)NHCHCl₂ | Z-353 | —Me | —C(═O)NHCHCl₂ | Z-354 | —Et | —C(═O)NHCHCl₂ |
| Z-355 | —H | —C(═O)NHCCl₃ | Z-356 | —Me | —C(═O)NHCCl₃ | Z-357 | —Et | —C(═O)NHCCl₃ |
| Z-358 | —H | —C(═O)NHCH₂Br | Z-359 | —Me | —C(═O)NHCH₂Br | Z-360 | —Et | —C(═O)NHCH₂Br |
| Z-361 | —H | —C(═O)NHCHBr₂ | Z-362 | —Me | —C(═O)NHCHBr₂ | Z-363 | —Et | —C(═O)NHCHBr₂ |
| Z-364 | —H | —C(═O)NHCBr₃ | Z-365 | —Me | —C(═O)NHCBr₃ | Z-366 | —Et | —C(═O)NHCBr₃ |
| Z-367 | —H | —C(═O)NHCH₂l | Z-368 | —Me | —C(═O)NHCH₂l | Z-369 | —Et | —C(═O)NHCH₂l |
| Z-370 | —H | —C(═O)NHCHl₂ | Z-371 | —Me | —C(═O)NHCHl₂ | Z-372 | —Et | —C(═O)NHCHl₂ |
| Z-373 | —H | —C(═O)NHCH₂CF₂H | Z-374 | —Me | —C(═O)NHCH₂CF₂H | Z-375 | —Et | —C(═O)NHCH₂CF₂H |
| Z-376 | —H | —C(═O)NHCH₂CF₃ | Z-377 | —Me | —C(═O)NHCH₂CF₃ | Z-378 | —Et | —C(═O)NHCH₂CF₃ |
| Z-379 | —H | —C(═O)NHCH₂CH₂CF₂H | Z-380 | —Me | —C(═O)NHCH₂CH₂CF₂H | Z-381 | —Et | —C(═O)NHCH₂CH₂CF₂H |
| Z-382 | —H | —C(═O)NHCH₂CH₂CF₃ | Z-383 | —Me | —C(═O)NHCH₂CH₂CF₃ | Z-384 | —Et | —C(═O)NHCH₂CH₂CF₃ |
| Z-385 | —H | —C(═O)NHCH₂CH₂CH₂CF₂H | Z-386 | —Me | —C(═O)NHCH₂CH₂CH₂CF₂H | Z-387 | —Et | —C(═O)NHCH₂CH₂CH₂CF₂H |
| Z-388 | —H | —C(═O)NHCH₂CH₂CH₂CF₃ | Z-389 | —Me | —C(═O)NHCH₂CH₂CH₂CF₃ | Z-390 | —Et | —C(═O)NHCH₂CH₂CH₂CF₃ |
| Z-391 | —H | —C(═O)NHCF₂CF₂H | Z-392 | —Me | —C(═O)NHCF₂CF₂H | Z-393 | —Et | —C(═O)NHCF₂CF₂H |
| Z-394 | —H | —C(═O)NHCF₂CF₃ | Z-395 | —Me | —C(═O)NHCF₂CF₃ | Z-396 | —Et | —C(═O)NHCF₂CF₃ |
| Z-397 | —H | —C(═O)NHCFHCF₃ | Z-398 | —Me | —C(═O)NHCFHCF₃ | Z-399 | —Et | —C(═O)NHCFHCF₃ |
| Z-400 | —H | —C(═O)NHCH₂CF₂CF₂H | Z-401 | —Me | —C(═O)NHCH₂CF₂CF₂H | Z-402 | —Et | —C(═O)NHCH₂CF₂CF₂H |
| Z-403 | —H | —C(═O)NHCH₂CF₂CF₃ | Z-404 | —Me | —C(═O)NHCH₂CF₂CF₃ | Z-405 | —Et | —C(═O)NHCH₂CF₂CF₃ |
| Z-406 | —H | —C(═O)NHCF₂CF₂CF₃ | Z-407 | —Me | —C(═O)NHCF₂CF₂CF₃ | Z-408 | —Et | —C(═O)NHCF₂CF₂CF₃ |
| Z-409 | —H | —C(═O)NHCH₂CF₂CF₂CF₃ | Z-410 | —Me | —C(═O)NHCH₂CF₂CF₂CF₃ | Z-411 | —Et | —C(═O)NHCH₂CF₂CF₂CF₃ |
| Z-412 | —H | —C(═O)NHCF₂CF₂CF₂CF₃ | Z-413 | —Me | —C(═O)NHCF₂CF₂CF₂CF₃ | Z-414 | —Et | —C(═O)NHCF₂CF₂CF₂CF₃ |
| Z-415 | —H | —C(═O)NHCH₂CF₂CF₂CF₂CF₃ | Z-416 | —Me | —C(═O)NHCH₂CF₂CF₂CF₂CF₃ | Z-417 | —Et | —C(═O)NHCH₂CF₂CF₂CF₂CF₃ |
| Z-418 | —H | —C(═O)NH—c-Pr | Z-419 | —Me | —C(═O)NH—c-Pr | Z-420 | —Et | —C(═O)NH—c-Pr |
| Z-421 | —H | —C(═O)NH—c-Bu | Z-422 | —Me | —C(═O)NH—c-Bu | Z-423 | —Et | —C(═O)NH—c-Bu |
| Z-424 | —H | —C(═O)NH—c-Pent | Z-425 | —Me | —C(═O)NH—c-Pent | Z-426 | —Et | —C(═O)NH—c-Pent |
| Z-427 | —H | —C(═O)NH—c-Hex | Z-428 | —Me | —C(═O)NH—c-Hex | Z-429 | —Et | —C(═O)NH—c-Hex |
| Z-430 | —H | —C(═O)NH—c-Hept | Z-431 | —Me | —C(═O)NH—c-Hept | Z-432 | —Et | —C(═O)NH—c-Hept |
| Z-433 | —H | —C(═O)NH—c-Oct | Z-434 | —Me | —C(═O)NH—c-Oct | Z-435 | —Et | —C(═O)NH—c-Oct |
| Z-436 | —H | —C(═O)NHCH₂CH═CH₂ | Z-437 | —Me | —C(═O)NHCH₂CH═CH₂ | Z-438 | —Et | —C(═O)NHCH₂CH═CH₂ |
| Z-439 | —H | —C(═O)NHCH₂C(CH₃)═CH₂ | Z-440 | —Me | —C(═O)NHCH₂C(CH₃)═CH₂ | Z-441 | —Et | —C(═O)NHCH₂C(CH₃)═CH₂ |
| Z-442 | —H | —C(═O)NHCH₂CH₂CH═CH₂ | Z-443 | —Me | —C(═O)NHCH₂CH₂CH═CH₂ | Z-444 | —Et | —C(═O)NHCH₂CH₂CH═CH₂ |
| Z-445 | —H | —C(═O)NHCH₂CH═CHCH₃ | Z-446 | —Me | —C(═O)NHCH₂CH═CHCH₃ | Z-447 | —Et | —C(═O)NHCH₂CH═CHCH₃ |
| Z-448 | —H | —C(═O)NHCH₂CH═C(CH₃)₂ | Z-449 | —Me | —C(═O)NHCH₂CH═C(CH₃)₂ | Z-450 | —Et | —C(═O)NHCH₂CH═C(CH₃)₂ |
| Z-451 | —H | —C(═O)NHCH₂CH₂CH═C(CH₃)₂ | Z-452 | —Me | —C(═O)NHCH₂CH₂CH═C(CH₃)₂ | Z-453 | —Et | —C(═O)NHCH₂CH₂CH═C(CH₃)₂ |
| Z-454 | —H | —C(═O)NHCH₂CH═CFH | Z-455 | —Me | —C(═O)NHCH₂CH═CFH | Z-456 | —Et | —C(═O)NHCH₂CH═CFH |
| Z-457 | —H | —C(═O)NHCH₂CH═CF₂ | Z-458 | —Me | —C(═O)NHCH₂CH═CF₂ | Z-459 | —Et | —C(═O)NHCH₂CH═CF₂ |
| Z-460 | —H | —C(═O)NHCH₂CH═CCl₂ | Z-461 | —Me | —C(═O)NHCH₂CH═CCl₂ | Z-462 | —Et | —C(═O)NHCH₂CH═CCl₂ |
| Z-463 | —H | —C(═O)NHCH₂CH₂CH═CF₂ | Z-464 | —Me | —C(═O)NHCH₂CH₂CH═CF₂ | Z-465 | —Et | —C(═O)NHCH₂CH₂CH═CF₂ |
| Z-466 | —H | —C(═O)NHCH₂CH₂CH₂CH═CF₂ | Z-467 | —Me | —C(═O)NHCH₂CH₂CH₂CH═CF₂ | Z-468 | —Et | —C(═O)NHCH₂CH₂CH₂CH═CF₂ |
| Z-469 | —H | —C(═O)NHCH₂CH₂CH₂CH₂CH═CF₂ | Z-470 | —Me | —C(═O)NHCH₂CH₂CH₂CH₂CH═CF₂ | Z-471 | —Et | —C(═O)NHCH₂CH₂CH₂CH₂CH═CF₂ |
| Z-472 | —H | —C(═O)NHCH₂C≡CH | Z-473 | —Me | —C(═O)NHCH₂C≡CH | Z-474 | —Et | —C(═O)NHCH₂C≡CH |
| Z-475 | —H | —C(═O)NHCH₂C≡CCH₃ | Z-476 | —Me | —C(═O)NHCH₂C≡CCH₃ | Z-477 | —Et | —C(═O)NHCH₂C≡CCH₃ |
| Z-478 | —H | —C(═O)NHCH₂CH₂C≡CH | Z-479 | —Me | —C(═O)NHCH₂CH₂C≡CH | Z-480 | —Et | —C(═O)NHCH₂CH₂C≡CH |
| Z-481 | —H | —C(═O)NHCH₂C≡CCH₂CH₃ | Z-482 | —Me | —C(═O)NHCH₂C≡CCH₂CH₃ | Z-483 | —Et | —C(═O)NHCH₂C≡CCH₂CH₃ |
| Z-484 | —H | —C(═O)NHC(CH₃)₂C≡CH | 2-485 | —Me | —C(═O)NHC(CH₃)₂C≡CH | Z-486 | —Et | —C(═O)NHC(CH₃)₂C≡CH |
| Z-487 | —H | —C(═O)NHCH₂C≡CHCF₂H | Z-488 | —Me | —C(═O)NHCH₂C≡CHCF₂H | Z-489 | —Et | —C(═O)NHCH₂C≡CHCF₂H |
| Z-490 | —H | —C(═O)NHCH₂C≡CCF₃ | Z-491 | —Me | —C(═O)NHCH₂C≡CCF₃ | Z-492 | —Et | —C(═O)NHCH₂C≡CCF₃ |
| Z-493 | —H | —C(═O)H | Z-494 | —Me | —C(═O)H | Z-495 | —Et | —C(═O)H |
| Z-496 | —H | —C(═O)Me | Z-497 | —Me | —C(═O)Me | Z-498 | —Et | —C(═O)Me |
| Z-499 | —H | —C(═O)Et | Z-500 | —Me | —C(═O)Et | Z-501 | —Et | —C(═O)Et |
| Z-502 | —H | —C(═O)Pr | Z-503 | —Me | —C(═O)Pr | Z-504 | —Et | —C(═O)Pr |
| Z-505 | —H | —C(═O)—i-Pr | 2-506 | —Me | —C(═O)—i-Pr | Z-507 | —Et | —C(═O)—i-Pr |
| Z-508 | —H | —C(═O)Bu | Z-509 | —Me | —C(═O)Bu | Z-510 | —Et | —C(═O)Bu |
| Z-511 | —H | —C(═O)—sec-Bu | Z-512 | —Me | —C(═O)—sec-Bu | Z-513 | —Et | —C(═O)—sec-Bu |
| Z-514 | —H | —C(═O)—i-Bu | Z-515 | —Me | —C(═O)—i-Bu | Z-516 | —Et | —C(═O)—i-Bu |
| Z-517 | —H | —C(═O)—t-Bu | Z-518 | —Me | —C(═O)—t-Bu | Z-519 | —Et | —C(═O)—t-Bu |
| Z-520 | —H | —C(═O)Pent | Z-521 | —Me | —C(═O)Pent | Z-522 | —Et | —C(═O)Pent |
| Z-523 | —H | —C(═O)Hex | Z-524 | —Me | —C(═O)Hex | Z-525 | —Et | —C(═O)Hex |
| Z-526 | —H | —C(═O)CH(CH₃)CH₂CH₂CH₃ | Z-527 | —Me | —C(═O)CH(CH₃)CH₂CH₂CH₃ | Z-528 | —Et | —C(═O)CH(CH₃)CH₂CH₂CH₃ |
| Z-529 | —H | —C(═O)CH(CH₃)CH(CH₃)₂ | Z-530 | —Me | —C(═O)CH(CH₃)CH(CH₃)₂ | Z-531 | —Et | —C(═O)CH(CH₃)CH(CH₃)₂ |
| 2-532 | —H | —C(═O)C(CH₃)₂CH₂CH₃ | Z-533 | —Me | —C(═O)C(CH₃)₂CH₂CH₃ | Z-534 | —Et | —C(═O)C(CH₃)₂CH₂CH₃ |
| Z-535 | —H | —C(═O)CH(CH₂CH₃)₂ | Z-536 | —Me | —C(═O)CH(CH₂CH₃)₂ | Z-537 | —Et | —C(═O)CH(CH₂CH₃)₂ |
| Z-538 | —H | —C(═O)CH₂CH₂CH(CH₃)₂ | Z-539 | —Me | —C(═O)CH₂CH₂CH(CH₃)₂ | Z-540 | —Et | —C(═O)CH₂CH₂CH(CH₃)₂ |
| Z-541 | —H | —C(═O)CH₂CH₂CH₂CH(CH₃)₂ | Z-542 | —Me | —C(═O)CH₂CH₂CH₂CH(CH₃)₂ | Z-543 | —Et | —C(═O)CH₂CH₂CH₂CH(CH₃)₂ |
| Z-544 | —H | —C(═O)CH₂CH₂CH(CH₃)CH₂CH₃ | Z-545 | —Me | —C(═O)CH₂CH₂CH(CH₃)CH₂CH₃ | Z-546 | —Et | —C(═O)CH₂CH₂CH(CH₃)CH₂CH₃ |
| Z-547 | —H | —C(═O)CH₂CH(CH₃)CH₂CH₂CH₃ | Z-548 | —Me | —C(═O)CH₂CH(CH₃)CH₂CH₂CH₃ | Z-549 | —Et | —C(═O)CH₂CH(CH₃)CH₂CH₂CH₃ |
| Z-550 | —H | —C(═O)CH(CH₃)CH₂CH₂CH₂CH₃ | Z-551 | —Me | —C(═O)CH(CH₃)CH₂CH₂CH₂CH₃ | Z-552 | —Et | —C(═O)CH(CH₃)CH₂CH₂CH₂CH₃ |
| Z-553 | —H | —C(═O)CH(CH₃)CH₂CH(CH₃)₂ | Z-554 | —Me | —C(═O)CH(CH₃)CH₂CH(CH₃)₂ | Z-555 | —Et | —C(═O)CH(CH₃)CH₂CH(CH₃)₂ |
| Z-556 | —H | —C(═O)CH(CH₃)CH(CH₃)CH₂CH₃ | Z-557 | —Me | —C(═O)CH(CH₃)CH(CH₃)CH₂CH₃ | Z-558 | —Et | —C(═O)CH(CH₃)CH(CH₃)CH₂CH₃ |
| Z-559 | —H | —C(═O)C(CH₃)₂CH₂CH₂CH₃ | Z-560 | —Me | —C(═O)C(CH₃)₂CH₂CH₂CH₃ | Z-561 | —Et | —C(═O)C(CH₃)₂CH₂CH₂CH₃ |
| Z-562 | —H | —C(═O)CH(CH₃)C(CH₃)₃ | Z-563 | —Me | —C(═O)CH(CH₃)C(CH₃)₃ | Z-564 | —Et | —C(═O)CH(CH₃)C(CH₃)₃ |
| Z-565 | —H | —C(═O)C(CH₃)₂CH(CH₃)₂ | Z-566 | —Me | —C(═O)C(CH₃)₂CH(CH₃)₂ | Z-567 | —Et | —C(═O)C(CH₃)₂CH(CH₃)₂ |
| Z-568 | —H | —C(═O)CH₂CH₂C(CH₃) | Z-569 | —Me | —C(═O)CH₂CH₂C(CH₃) | Z-570 | —Et | —C(═O)CH₂CH₂C(CH₃) |
| Z-571 | —H | —C(═O)CH₂CH(CH₃)CH(CH₃)₂ | Z-572 | —Me | —C(═O)CH₂CH(CH₃)CH(CH₃)₂ | Z-573 | —Et | —C(═O)CH₂CH(CH₃)CH(CH₃)₂ |
| Z-574 | —H | —C(═O)CH₂C(CH₃)₂CH₂CH₃ | Z-575 | —Me | —C(═O)CH₂C(CH₃)₂CH₂CH₃ | Z-576 | —Et | —C(═O)CH₂C(CH₃)₂CH₂CH₃ |
| Z-577 | —H | —C(═O)CFH₂ | Z-578 | —Me | —C(═O)CFH₂ | Z-579 | —Et | —C(═O)CFH₂ |
| Z-580 | —H | —C(═O)CF₂H | Z-581 | —Me | —C(═O)CF₂H | Z-582 | —Et | —C(═O)CF₂H |
| Z-583 | —H | —C(═O)CF₂Cl | Z-584 | —Me | —C(═O)CF₂Cl | Z-585 | —Et | —C(═O)CF₂Cl |
| Z-586 | —H | —C(═O)CF₃ | Z-587 | —Me | —C(═O)CF₃ | Z-588 | —Et | —C(═O)CF₃ |
| Z-589 | —H | —C(═O)CH₂Cl | Z-590 | —Me | —C(═O)CH₂Cl | Z-591 | —Et | —C(═O)CH₂Cl |
| Z-592 | —H | —C(═O)CHCl₂ | Z-593 | —Me | —C(═O)CHCl₂ | Z-594 | —Et | —C(═O)CHCl₂ |
| Z-595 | —H | —C(═O)CCl₂F | Z-596 | —Me | —C(═O)CCl₂F | Z-597 | —Et | —C(═O)CCl₂F |
| Z-598 | —H | —C(═O)CCl₃ | Z-599 | —Me | —C(═O)CCl₃ | Z-600 | —Et | —C(═O)CCl₃ |
| Z-601 | —H | —C(═O)CH₂Br | Z-602 | —Me | —C(═O)CH₂Br | Z-603 | —Et | —C(═O)CH₂Br |
| Z-604 | —H | —C(═O)CHBr₂ | Z-605 | —Me | —C(═O)CHBr₂ | Z-606 | —Et | —C(═O)CHBr₂ |
| Z-607 | —H | —C(═O)CBr₃ | Z-608 | —Me | —C(═O)CBr₃ | Z-609 | —Et | —C(═O)CBr₃ |
| Z-610 | —H | —C(═O)CH₂l | Z-611 | —Me | —C(═O)CH₂l | Z-612 | —Et | —C(═O)CH₂l |
| Z-613 | —H | —C(═O)CHl₂ | Z-614 | —Me | —C(═O)CHl₂ | Z-615 | —Et | —C(═O)CHl₂ |
| Z-616 | —H | —C(═O)CH₂CF₂H | Z-617 | —Me | —C(═O)CH₂CF₂H | Z-618 | —Et | —C(═O)CH₂CF₂H |
| Z-619 | —H | —C(═O)CH₂CF₃ | Z-620 | —Me | —C(═O)CH₂CF₃ | Z-621 | —Et | —C(═O)CH₂CF₃ |
| Z-622 | —H | —C(═O)CH₂CH₂CF₂H | Z-623 | —Me | —C(═O)CH₂CH₂CF₂H | Z-624 | —Et | —C(═O)CH₂CH₂CF₂H |
| Z-625 | —H | —C(═O)CH₂CH₂CF₃ | Z-626 | —Me | —C(═O)CH₂CH₂CF₃ | Z-627 | —Et | —C(═O)CH₂CH₂CF₃ |
| Z-628 | —H | —C(═O)CH₂CH₂CH₂CF₂H | Z-629 | —Me | —C(═O)CH₂CH₂CH₂CF₂H | Z-630 | —Et | —C(═O)CH₂CH₂CH₂CF₂H |
| Z-631 | —H | —C(═O)CH₂CH₂CH₂CF₃ | Z-632 | —Me | —C(═O)CH₂CH₂CH₂CF₃ | Z-633 | —Et | —C(═O)CH₂CH₂CH₂CF₃ |
| Z-634 | —H | —C(═O)CF₂CH₃ | Z-635 | —Me | —C(═O)CF₂CH₃ | Z-636 | —Et | —C(═O)CF₂CH₃ |
| Z-637 | —H | —C(═O)CF₂CF₂H | Z-638 | —Me | —C(═O)CF₂CF₂H | Z-639 | —Et | —C(═O)CF₂CF₂H |
| Z-640 | —H | —C(═O)CF₂CF₃ | Z-641 | —Me | —C(═O)CF₂CF₃ | Z-642 | —Et | —C(═O)CF₂CF₃ |
| Z-643 | —H | —C(═O)CF₂CClF₂ | Z-644 | —Me | —C(═O)CF₂CClF₂ | Z-645 | —Et | —C(═O)CF₂CClF₂ |
| Z-646 | —H | —C(═O)CFHCF₃ | Z-647 | —Me | —C(═O)CFHCF₃ | Z-648 | —Et | —C(═O)CFHCF₃ |
| Z-649 | —H | —C(═O)CH₂CF₂CF₂H | Z-650 | —Me | —C(═O)CH₂CF₂CF₂H | Z-651 | —Et | —C(═O)CH₂CF₂CF₂H |
| Z-652 | —H | —C(═O)CH₂CF₂CF₃ | Z-653 | —Me | —C(═O)CH₂CF₂CF₃ | Z-654 | —Et | —C(═O)CH₂CF₂CF₃ |
| Z-655 | —H | —C(═O)CF₂CF₂CF₃ | Z-656 | —Me | —C(═O)CF₂CF₂CF₃ | Z-657 | —Et | —C(═O)CF₂CF₂CF₃ |
| Z-658 | —H | —C(═O)CH₂CF₂CF₂CF₃ | Z-659 | —Me | —C(═O)CH₂CF₂CF₂CF₃ | Z-660 | —Et | —C(═O)CH₂CF₂CF₂CF₃ |
| Z-661 | —H | —C(═O)CF₂CF₂CF₂CF₃ | Z-662 | —Me | —C(═O)CF₂CF₂CF₂CF₃ | Z-663 | —Et | —C(═O)CF₂CF₂CF₂CF₃ |
| Z-664 | —H | —C(═O)CH₂CF₂CF₂CF₂CF₃ | Z-665 | —Me | —C(═O)CH₂CF₂CF₂CF₂CF₃ | Z-666 | —Et | —C(═O)CH₂CF₂CF₂CF₂CF₃ |
| Z-667 | —H | —C(═O)CF₂CF₂CF₂CF₂CF₃ | Z-668 | —Me | —C(═O)CF₂CF₂CF₂CF₂CF₃ | Z-669 | —Et | —C(═O)CF₂CF₂CF₂CF₂CF₃ |
| Z-670 | —H | —C(═O)—c-Pr | Z-671 | —Me | —C(═O)—c-Pr | Z-672 | —Et | —C(═O)—c-Pr |
| Z-673 | —H | —C(═O)—c-Bu | Z-674 | —Me | —C(═O)—c-Bu | Z-675 | —Et | —C(═O)—c-Bu |
| Z-676 | —H | —C(═O)—c-Pent | Z-677 | —Me | —C(═O)—c-Pent | Z-678 | —Et | —C(═O)—c-Pent |
| Z-679 | —H | —C(═O)—c-Hex | Z-680 | —Me | —C(═O)—c-Hex | Z-681 | —Et | —C(═O)—c-Hex |
| Z-682 | —H | —C(═O)—c-Hept | Z-683 | —Me | —C(═O)—c-Hept | Z-684 | —Et | —C(═O)—c-Hept |
| Z-685 | —H | —C(═O)—c-Oct | Z-686 | —Me | —C(═O)—c-Oct | Z-687 | —Et | —C(═O)—c-Oct |
| Z-688 | —H | —C(═O)CH═CH₂ | Z-689 | —Me | —C(═O)CH═CH₂ | Z-690 | —Et | —C(═O)CH═CH₂ |
| Z-691 | —H | —C(═O)CH₂CH═CH₂ | Z-692 | —Me | —C(═O)CH₂CH═CH₂ | Z-693 | —Et | —C(═O)CH₂CH═CH₂ |
| Z-694 | —H | —C(═O)CH═CHCH₃ | Z-695 | —Me | —C(═O)CH═CHCH₃ | Z-696 | —Et | —C(═O)CH═CHCH₃ |
| Z-697 | —H | —C(═O)CH₂C(CH₃)═CH₂ | Z-698 | —Me | —C(═O)CH₂C(CH₃)═CH₂ | Z-699 | —Et | —C(═O)CH₂C(CH₃)═CH₂ |
| Z-700 | —H | —C(═O)CH₂CH₂CH═CH₂ | Z-701 | —Me | —C(═O)CH₂CH₂CH═CH₂ | Z-702 | —Et | —C(═O)CH₂CH₂CH═CH₂ |
| Z-703 | —H | —C(═O)CH₂CH═CHCH₃ | Z-704 | —Me | —C(═O)CH₂CH═CHCH₃ | Z-705 | —Et | —C(═O)CH₂CH═CHCH₃ |
| Z-706 | —H | —C(═O)CH═CHCH₂CH₃ | Z-707 | —Me | —C(═O)CH═CHCH₂CH₃ | Z-708 | —Et | —C(═O)CH═CHCH₂CH₃ |
| Z-709 | —H | —C(═O)CH₂CH═C(CH₃)₂ | Z-710 | —Me | —C(═O)CH₂CH═C(CH₃)₂ | Z-711 | —Et | —C(═O)CH₂CH═C(CH₃)₂ |
| Z-712 | —H | —C(═O)CH₂CH₂CH═C(CH₃)₂ | Z-713 | —Me | —C(═O)CH₂CH₂CH═C(CH₃)₂ | Z-714 | —Et | —C(═O)CH₂CH₂CH═C(CH₃)₂ |
| Z-715 | —H | —C(═O)CH═CFH | Z-716 | —Me | —C(═O)CH═CFH | Z-717 | —Et | —C(═O)CH═CFH |
| Z-718 | —H | —C(═O)CH═CF₂ | Z-719 | —Me | —C(═O)CH═CF₂ | Z-720 | —Et | —C(═O)CH═CF₂ |
| Z-721 | —H | —C(═O)CH═CCl₂ | Z-722 | —Me | —C(═O)CH═CCl₂ | Z-723 | —Et | —C(═O)CH═CCl₂ |
| Z-724 | —H | —C(═O)CH₂CH═CFH | Z-725 | —Me | —C(═O)CH₂CH═CFH | Z-726 | —Et | —C(═O)CH₂CH═CFH |
| Z-727 | —H | —C(═O)CH₂CH═CF₂ | Z-728 | —Me | —C(═O)CH₂CH═CF₂ | Z-729 | —Et | —C(═O)CH₂CH═CF₂ |
| Z-730 | —H | —C(═O)CH₂CH═CCl₂ | Z-731 | —Me | —C(═O)CH₂CH═CCl₂ | Z-732 | —Et | —C(═O)CH₂CH═CCl₂ |
| Z-733 | —H | —C(═O)CH₂CH₂CH═CF₂ | Z-734 | —Me | —C(═O)CH₂CH₂CH═CF₂ | Z-735 | —Et | —C(═O)CH₂CH₂CH═CF₂ |
| Z-736 | —H | —C(═O)CH₂CH₂CH₂CH═CF₂ | Z-737 | —Me | —C(═O)CH₂CH₂CH₂CH═CF₂ | Z-738 | —Et | —C(═O)CH₂CH₂CH₂CH═CF₂ |
| Z-739 | —H | —C(═O)CH₂CH₂CH₂CH₂CH═CF₂ | Z-740 | —Me | —C(═O)CH₂CH₂CH₂CH₂CH═CF₂ | Z-741 | —Et | —C(═O)CH₂CH₂CH₂CH₂CH═CF₂ |
| Z-742 | —H | —C(═O)C≡CH | Z-743 | —Me | —C(═O)C≡CH | Z-744 | —Et | —C(═O)C≡CH |
| Z-745 | —H | —C(═O)C≡CCH₃ | Z-746 | —Me | —C(═O)C≡CCH₃ | Z-747 | —Et | —C(═O)C≡CCH₃ |
| Z-748 | —H | —C(═O)CH₂C≡CH | Z-749 | —Me | —C(═O)CH₂C≡CH | Z-750 | —Et | —C(═O)CH₂C≡CH |
| Z-751 | —H | —C(═O)C≡CCH₂CH₃ | Z-752 | —Me | —C(═O)C≡CCH₂CH₃ | Z-753 | —Et | —C(═O)C≡CCH₂CH₃ |
| Z-754 | —H | —C(═O)CH₂C≡CCH₃ | Z-755 | —Me | —C(═O)CH₂C≡CCH₃ | Z-756 | —Et | —C(═O)CH₂C≡CCH₃ |
| Z-757 | —H | —C(═O)CH₂CH₂C≡CH | Z-758 | —Me | —C(═O)CH₂CH₂C≡CH | Z-759 | —Et | —C(═O)CH₂CH₂C≡CH |
| Z-760 | —H | —C(═O)C≡CCH₂CH₂CH₃ | Z-761 | —Me | —C(═O)C≡CCH₂CH₂CH₃ | Z-762 | —Et | —C(═O)C≡CCH₂CH₂CH₃ |
| Z-763 | —H | —C(═O)CH₂C≡CCH₂CH₃ | Z-764 | —Me | —C(═O)CH₂C≡CCH₂CH₃ | Z-765 | —Et | —C(═O)CH₂C≡CCH₂CH₃ |
| Z-766 | —H | —C(═O)C(CH₃)₂C≡CH | Z-767 | —Me | —C(═O)C(CH₃)₂C≡CH | Z-768 | —Et | —C(═O)C(CH₃)₂C≡CH |
| Z-769 | —H | —C(═O)C≡CF | Z-770 | —Me | —C(═O)C≡CF | Z-771 | —Et | —C(═O)C≡CF |
| Z-772 | —H | —C(═O)C≡CCF₂H | Z-773 | —Me | —C(═O)C≡CCF₂H | Z-774 | —Et | —C(═O)C≡CCF₂H |
| Z-775 | —H | —C(═O)C≡CCF₃ | Z-776 | —Me | —C(═O)C≡CCF₃ | Z-777 | —Et | —C(═O)C≡CCF₃ |
| Z-778 | —H | —C(═O)C≡CCH₂CF₂H | Z-779 | —Me | —C(═O)C≡CCH₂CF₂H | Z-780 | —Et | —C(═O)C≡CCH₂CF₂H |
| Z-781 | —H | —C(═O)C≡CCH₂CF₃ | Z-782 | —Me | —C(═O)C≡CCH₂CF₃ | Z-783 | —Et | —C(═O)C≡CCH₂CF₃ |
| Z-784 | —H | —C(═O)CH₂C≡CHCF₂H | Z-785 | —Me | —C(═O)CH₂C≡CHCF₂H | Z-786 | —Et | —C(═O)CH₂C≡CHCF₂H |
| Z-787 | —H | —C(═O)CH₂C≡CCF₃ | Z-788 | —Me | —C(═O)CH₂C≡CCF₃ | Z-789 | —Et | —C(═O)CH₂C≡CCF₃ |
| Z-790 | —H | —C(═O)CH₂C≡N | Z-791 | —Me | —C(═O)CH₂C≡N | Z-792 | —Et | —C(═O)CH₂C≡N |
| Z-793 | —H | —C(═O)C(Me)C≡N | Z-794 | —Me | —C(═O)C(Me)C≡N | Z-795 | —Et | —C(═O)C(Me)C≡N |
| Z-796 | —H | —C(═O)CH₂CH₂C≡N | Z-797 | —Me | —C(═O)CH₂CH₂C≡N | Z-798 | —Et | —C(═O)CH₂CH₂C≡N |
| Z-799 | —H | —C(═O)CH₂CH₂CH₂C≡N | Z-800 | —Me | —C(═O)CH₂CH₂CH₂C≡N | Z-801 | —Et | —C(═O)CH₂CH₂CH₂C≡N |
| Z-802 | —H | —C(═O)CH₂OH | Z-803 | —Me | —C(═O)CH₂OH | Z-804 | —Et | —C(═O)CH₂OH |
| Z-805 | —H | —C(═O)CH₂OMe | Z-806 | —Me | —C(═O)CH₂OMe | Z-807 | —Et | —C(═O)CH₂OMe |
| Z-808 | —H | —C(═O)CH₂OEt | Z-809 | —Me | —C(═O)CH₂OEt | Z-810 | —Et | —C(═O)CH₂OEt |
| Z-811 | —H | —C(═O)CH₂OPr | Z-812 | —Me | —C(═O)CH₂OPr | Z-813 | —Et | —C(═O)CH₂OPr |
| Z-814 | —H | —C(═O)CH₂CH₂OMe | Z-815 | —Me | —C(═O)CH₂CH₂OMe | Z-816 | —Et | —C(═O)CH₂CH₂OMe |
| Z-817 | —H | —C(═O)CH₂CH₂OEt | Z-818 | —Me | —C(═O)CH₂CH₂OEt | Z-819 | —Et | —C(═O)CH₂CH₂OEt |
| Z-820 | —H | —C(═O)CH₂—(1-Pyra) | Z-821 | —Me | —C(═O)CH₂—(1-Pyra) | Z-822 | —Et | —C(═O)CH₂—(1-Pyra) |
| Z-823 | —H | —C(═O)CH₂—(1-Tria) | Z-824 | —Me | —C(═O)CH₂—(1-Tria) | Z-825 | —Et | —C(═O)CH₂—(1-Tria) |
| Z-826 | —H | —C(═O)OH | Z-827 | —Me | —C(═O)OH | Z-828 | —Et | —C(═O)OH |
| Z-829 | —H | —C(═O)OMe | Z-830 | —Me | —C(═O)OMe | Z-831 | —Et | —C(═O)OMe |
| Z-832 | —H | —C(═O)OEt | Z-833 | —Me | —C(═O)OEt | Z-834 | —Et | —C(═O)OEt |
| Z-835 | —H | —C(═O)OPr | Z-836 | —Me | —C(═O)OPr | Z-837 | —Et | —C(═O)OPr |
| Z-838 | —H | —C(═O)O—i-Pr | Z-839 | —Me | —C(═O)O—i-Pr | Z-840 | —Et | —C(═O)O—i-Pr |
| Z-841 | —H | —C(═O)OBu | Z-842 | —Me | —C(═O)OBu | Z-843 | —Et | —C(═O)OBu |
| Z-844 | —H | —C(═O)O—sec-Bu | Z-845 | —Me | —C(═O)O—sec-Bu | Z-846 | —Et | —C(═O)O—sec-Bu |
| Z-847 | —H | —C(═O)O—i-Bu | Z-848 | —Me | —C(═O)O—i-Bu | Z-849 | —Et | —C(═O)O—i-Bu |
| Z-850 | —H | —C(═O)O—t-Bu | Z-851 | —Me | —C(═O)O—t-Bu | Z-852 | —Et | —C(═O)O—t-Bu |
| Z-853 | —H | —C(═O)OPent | Z-854 | —Me | —C(═O)OPent | Z-855 | —Et | —C(═O)OPent |
| Z-856 | —H | —C(═O)OHex | Z-857 | —Me | —C(═O)OHex | Z-858 | —Et | —C(═O)OHex |
| Z-859 | —H | —C(═O)OCH(CH₃)CH₂CH₂CH₃ | Z-860 | —Me | —C(═O)OCH(CH₃)CH₂CH₂CH₃ | Z-861 | —Et | —C(═O)OCH(CH₃)CH₂CH₂CH₃ |
| Z-862 | —H | —C(═O)OCH(CH₃)CH(CH₃)₂ | Z-863 | —Me | —C(═O)OCH(CH₃)CH(CH₃)₂ | Z-864 | —Et | —C(═O)OCH(CH₃)CH(CH₃)₂ |
| Z-865 | —H | —C(═O)OC(CH₃)₂CH₂CH₃ | Z-866 | —Me | —C(═O)OC(CH₃)₂CH₂CH₃ | Z-867 | —Et | —C(═O)OC(CH₃)₂CH₂CH₃ |
| Z-868 | —H | —C(═O)OCH(CH₂CH₃)₂ | Z-869 | —Me | —C(═O)OCH(CH₂CH₃)₂ | Z-870 | —Et | —C(═O)OCH(CH₂CH₃)₂ |
| Z-871 | —H | —C(═O)OCH₂CH₂CH(CH₃)₂ | Z-872 | —Me | —C(═O)OCH₂CH₂CH(CH₃)₂ | Z-873 | —Et | —C(═O)OCH₂CH₂CH(CH₃)₂ |
| Z-874 | —H | —C(═O)OCH₂CH₂CH₂CH(CH₃)₂ | Z-875 | —Me | —C(═O)OCH₂CH₂CH₂CH(CH₃)₂ | Z-876 | —Et | —C(═O)OCH₂CH₂CH₂CH(CH₃)₂ |
| Z-877 | —H | —C(═O)OCH₂CH₂CH(CH₃)CH₂CH₃ | Z-878 | —Me | —C(═O)OCH₂CH₂CH(CH₃)CH₂CH₃ | Z-879 | —Et | —C(═O)OCH₂CH₂CH(CH₃)CH₂CH₃ |
| Z-880 | —H | —C(═O)OCH₂CH(CH₃)CH₂CH₂CH₃ | Z-881 | —Me | —C(═O)OCH₂CH(CH₃)CH₂CH₂CH₃ | Z-882 | —Et | —C(═O)OCH₂CH(CH₃)CH₂CH₂CH₃ |
| Z-883 | —H | —C(═O)OCH(CH₃)CH₂CH₂CH₂CH₃ | Z-884 | —Me | —C(═O)OCH(CH₃)CH₂CH₂CH₂CH₃ | Z-885 | —Et | —C(═O)OCH(CH₃)CH₂CH₂CH₂CH₃ |
| Z-886 | —H | —C(═O)OCH(CH₃)CH₂CH(CH₃)₂ | Z-887 | —Me | —C(═O)OCH(CH₃)CH₂CH(CH₃)₂ | Z-888 | —Et | —C(═O)OCH(CH₃)CH₂CH(CH₃)₂ |
| Z-889 | —H | —C(═O)OCH(CH₃)CH(CH₃)CH₂CH₃ | Z-890 | —Me | —C(═O)OCH(CH₃)CH(CH₃)CH₂CH₃ | Z-891 | —Et | —C(═O)OCH(CH₃)CH(CH₃)CH₂CH₃ |
| Z-892 | —H | —C(═O)OC(CH₃)₂CH₂CH₂CH₃ | Z-893 | —Me | —C(═O)OC(CH₃)₂CH₂CH₂CH₃ | Z-894 | —Et | —C(═O)OC(CH₃)₂CH₂CH₂CH₃ |
| Z-895 | —H | —C(═O)OCH(CH₃)C(CH₃)₃ | Z-896 | —Me | —C(═O)OCH(CH₃)C(CH₃)₃ | Z-897 | —Et | —C(═O)OCH(CH₃)C(CH₃)₃ |
| Z-898 | —H | —C(═O)OC(CH₃)₂CH(CH₃)₂ | Z-899 | —Me | —C(═O)OC(CH₃)₂CH(CH₃)₂ | Z-900 | —Et | —C(═O)OC(CH₃)₂CH(CH₃)₂ |
| Z-901 | —H | —C(═O)OCH₂CH₂C(CH₃)₃ | Z-902 | —Me | —C(═O)OCH₂CH₂C(CH₃)₃ | Z-903 | —Et | —C(═O)OCH₂CH₂C(CH₃)₃ |
| Z-904 | —H | —C(═O)OCH₂CH(CH₃)CH(CH₃)₂ | Z-905 | —Me | —C(═O)OCH₂CH(CH₃)CH(CH₃)₂ | Z-906 | —Et | —C(═O)OCH₂CH(CH₃)CH(CH₃)₂ |
| Z-907 | —H | —C(═O)OCH₂C(CH₃)₂CH₂CH₃ | Z-908 | —Me | —C(═O)OCH₂C(CH₃)₂CH₂CH₃ | Z-909 | —Et | —C(═O)OCH₂C(CH₃)₂CH₂CH₃ |
| Z-910 | —H | —C(═O)OCFH₂ | Z-911 | —Me | —C(═O)OCFH₂ | Z-912 | —Et | —C(═O)OCFH₂ |
| Z-913 | —H | —C(═O)OCF₂H | Z-914 | —Me | —C(═O)OCF₂H | Z-915 | —Et | —C(═O)OCF₂H |
| Z-916 | —H | —C(═O)OCF₃ | Z-917 | —Me | —C(═O)OCF₃ | Z-918 | —Et | —C(═O)OCF₃ |
| Z-919 | —H | —C(═O)OCH₂Cl | Z-920 | —Me | —C(═O)OCH₂Cl | Z-921 | —Et | —C(═O)OCH₂Cl |
| Z-922 | —H | —C(═O)OCHCl₂ | Z-923 | —Me | —C(═O)OCHCl₂ | Z-924 | —Et | —C(═O)OCHCl₂ |
| Z-925 | —H | —C(═O)OCCl₃ | Z-926 | —Me | —C(═O)OCCl₃ | Z-927 | —Et | —C(═O)OCCl₃ |
| Z-928 | —H | —C(═O)OCH₂Br | Z-929 | —Me | —C(═O)OCH₂Br | Z-930 | —Et | —C(═O)OCH₂Br |
| Z-931 | —H | —C(═O)OCHBr₂ | Z-932 | —Me | —C(═O)OCHBr₂ | Z-933 | —Et | —C(═O)OCHBr₂ |
| Z-934 | —H | —C(═O)OCBr₃ | Z-935 | —Me | —C(═O)OCBr₃ | Z-936 | —Et | —C(═O)OCBr₃ |
| Z-937 | —H | —C(═O)OCH₂l | Z-938 | —Me | —C(═O)OCH₂l | Z-939 | —Et | —C(═O)OCH₂l |
| Z-940 | —H | —C(═O)OCHl₂ | Z-941 | —Me | —C(═O)OCHl₂ | Z-942 | —Et | —C(═O)OCHl₂ |
| Z-943 | —H | —C(═O)OCH₂CF₂H | Z-944 | —Me | —C(═O)OCH₂CF₂H | Z-945 | —Et | —C(═O)OCH₂CF₂H |
| Z-946 | —H | —C(═O)OCH₂CF₃ | Z-947 | —Me | —C(═O)OCH₂CF₃ | Z-948 | —Et | —C(═O)OCH₂CF₃ |
| Z-949 | —H | —C(═O)OCH₂CH₂CF₂H | Z-950 | —Me | —C(═O)OCH₂CH₂CF₂H | Z-951 | —Et | —C(═O)OCH₂CH₂CF₂H |
| Z-952 | —H | —C(═O)OCH₂CH₂CF₃ | Z-953 | —Me | —C(═O)OCH₂CH₂CF₃ | Z-954 | —Et | —C(═O)OCH₂CH₂CF₃ |
| Z-955 | —H | —C(═O)OCH₂CH₂CH₂CF₂H | Z-956 | —Me | —C(═O)OCH₂CH₂CH₂CF₂H | Z-957 | —Et | —C(═O)OCH₂CH₂CH₂CF₂H |
| Z-958 | —H | —C(═O)OCH₂CH₂CH₂CF₃ | Z-959 | —Me | —C(═O)OCH₂CH₂CH₂CF₃ | Z-960 | —Et | —C(═O)OCH₂CH₂CH₂CF₃ |
| Z-961 | —H | —C(═O)OCF₂CF₂H | Z-962 | —Me | —C(═O)OCF₂CF₂H | Z-963 | —Et | —C(═O)OCF₂CF₂H |
| Z-964 | —H | —C(═O)OCF₂CF₃ | Z-965 | —Me | —C(═O)OCF₂CF₃ | Z-966 | —Et | —C(═O)OCF₂CF₃ |
| Z-967 | —H | —C(═O)OCFHCF₃ | Z-968 | —Me | —C(═O)OCFHCF₃ | Z-969 | —Et | —C(═O)OCFHCF₃ |
| Z-970 | —H | —C(═O)OCH₂CF₂CF₂H | Z-971 | —Me | —C(═O)OCH₂CF₂CF₂H | Z-972 | —Et | —C(═O)OCH₂CF₂CF₂H |
| Z-973 | —H | —C(═O)OCH₂CF₂CF₃ | Z-974 | —Me | —C(═O)OCH₂CF₂CF₃ | Z-975 | —Et | —C(═O)OCH₂CF₂CF₃ |
| Z-976 | —H | —C(═O)OCF₂CF₂CF₃ | Z-977 | —Me | —C(═O)OCF₂CF₂CF₃ | Z-978 | —Et | —C(═O)OCF₂CF₂CF₃ |
| Z-979 | —H | —C(═O)OCH₂CF₂CF₂CF₃ | Z-980 | —Me | —C(═O)OCH₂CF₂CF₂CF₃ | Z-981 | —Et | —C(═O)OCH₂CF₂CF₂CF₃ |
| Z-982 | —H | —C(═O)OCF₂CF₂CF₂CF₃ | Z-983 | —Me | —C(═O)OCF₂CF₂CF₂CF₃ | Z-984 | —Et | —C(═O)OCF₂CF₂CF₂CF₃ |
| Z-985 | —H | —C(═O)OCH₂CF₂CF₂CF₂CF₃ | Z-986 | —Me | —C(═O)OCH₂CF₂CF₂CF₂CF₃ | Z-987 | —Et | —C(═O)OCH₂CF₂CF₂CF₂CF₃ |
| Z-988 | —H | —C(═O)O—c-Pr | Z-989 | —Me | —C(═O)O—c-Pr | Z-990 | —Et | —C(═O)O—c-Pr |
| Z-991 | —H | —C(═O)O—c-Bu | Z-992 | —Me | —C(═O)O—c-Bu | Z-993 | —Et | —C(═O)O—c-Bu |
| Z-994 | —H | —C(═O)O—c-Pent | Z-995 | —Me | —C(═O)O—c-Pent | Z-996 | —Et | —C(═O)O—c-Pent |
| Z-997 | —H | —C(═O)O—c-Hex | Z-998 | —Me | —C(═O)O—c-Hex | Z-999 | —Et | —C(═O)O—c-Hex |
| Z-1000 | —H | —C(═O)O—c-Hept | Z-1001 | —Me | —C(═O)O—c-Hept | Z-1002 | —Et | —C(═O)O—c-Hept |
| Z-1003 | —H | —C(═O)O—c-Oct | Z-1004 | —Me | —C(═O)O—c-Oct | Z-1005 | —Et | —C(═O)O—c-Oct |
| Z-1006 | —H | —C(═O)OCH═CH₂ | Z-1007 | —Me | —C(═O)OCH═CH₂ | Z-1008 | —Et | —C(═O)OCH═CH₂ |
| Z-1009 | —H | —C(═O)OCH₂CH═CH₂ | Z-1010 | —Me | —C(═O)OCH₂CH═CH₂ | Z-1011 | —Et | —C(═O)OCH₂CH═CH₂ |
| Z-1012 | —H | —C(═O)OCH═CHCH₃ | Z-1013 | —Me | —C(═O)OCH═CHCH₃ | Z-1014 | —Et | —C(═O)OCH═CHCH₃ |
| Z-1015 | —H | —C(═O)OCH₂C(CH₃)═CH₂ | Z-1016 | —Me | —C(═O)OCH₂C(CH₃)═CH₂ | Z-1017 | —Et | —C(═O)OCH₂C(CH₃)═CH₂ |
| Z-1018 | —H | —C(═O)OCH₂CH₂CH═CH₂ | Z-1019 | —Me | —C(═O)OCH₂CH₂CH═CH₂ | Z-1020 | —Et | —C(═O)OCH₂CH₂CH═CH₂ |
| Z-1021 | —H | —C(═O)OCH₂CH═CHCH₃ | Z-1022 | —Me | —C(═O)OCH₂CH═CHCH₃ | Z-1023 | —Et | —C(═O)OCH₂CH═CHCH₃ |
| Z-1024 | —H | —C(═O)OCH═CHCH₂CH₃ | Z-1025 | —Me | —C(═O)OCH═CHCH₂CH₃ | Z-1026 | —Et | —C(═O)OCH═CHCH₂CH₃ |
| Z-1027 | —H | —C(═O)OCH₂CH═C(CH₃)₂ | Z-1028 | —Me | —C(═O)OCH₂CH═C(CH₃)₂ | Z-1029 | —Et | —C(═O)OCH₂CH═C(CH₃)₂ |
| Z-1030 | —H | —C(═O)OCH2CH₂CH═C(CH₃)₂ | Z-1031 | —Me | —C(═O)OCH2CH₂CH═C(CH₃)₂ | Z-1032 | —Et | —C(═O)OCH2CH₂CH═C(CH₃)₂ |
| Z-1033 | —H | —C(═O)OCH═CFH | Z-1034 | —Me | —C(═O)OCH═CFH | Z-1035 | —Et | —C(═O)OCH═CFH |
| Z-1036 | —H | —C(═O)OCH═CF₂ | Z-1037 | —Me | —C(═O)OCH═CF₂ | Z-1038 | —Et | —C(═O)OCH═CF₂ |
| Z-1039 | —H | —C(═O)OCH═CCl₂ | Z-1040 | —Me | —C(═O)OCH═CCl₂ | Z-1041 | —Et | —C(═O)OCH═CCl₂ |
| Z-1042 | —H | —C(═O)OCH₂CH═CFH | Z-1043 | —Me | —C(═O)OCH₂CH═CFH | Z-1044 | —Et | —C(═O)OCH₂CH═CFH |
| Z-1045 | —H | —C(═O)OCH₂CH═CF₂ | Z-1046 | —Me | —C(═O)OCH₂CH═CF₂ | Z-1047 | —Et | —C(═O)OCH₂CH═CF₂ |
| Z-1048 | —H | —C(═O)OCH₂CH═CCl₂ | Z-1049 | —Me | —C(═O)OCH₂CH═CCl₂ | Z-1050 | —Et | —C(═O)OCH₂CH═CCl₂ |
| Z-1051 | —H | —C(═O)OCH₂CH₂CH═CF₂ | Z-1052 | —Me | —C(═O)OCH₂CH₂CH═CF₂ | Z-1053 | —Et | —C(═O)OCH₂CH₂CH═CF₂ |
| Z-1054 | —H | —C(═O)OCH₂CH₂CH₂CH═CF₂ | Z-1055 | —Me | —C(═O)OCH₂CH₂CH₂CH═CF₂ | Z-1056 | —Et | —C(═O)OCH₂CH₂CH₂CH═CF₂ |
| Z-1057 | —H | —C(═O)OCH₂CH₂CH₂CH₂CH═CF₂ | Z-1058 | —Me | —C(═O)OCH₂CH₂CH₂CH₂CH═CF₂ | Z-1059 | —Et | —C(═O)OCH₂CH₂CH₂CH₂CH═CF₂ |
| Z-1060 | —H | —C(═Q)OCH₂C≡CH | Z-1061 | —Me | —C(═Q)OCH₂C≡CH | Z-1062 | —Et | —C(═Q)OCH₂C≡CH |
| Z-1063 | —H | —C(═O)OCH₂C≡CCH₃ | Z-1064 | —Me | —C(═O)OCH₂C≡CCH₃ | Z-1065 | —Et | —C(═O)OCH₂C≡CCH₃ |
| Z-1066 | —H | —C(═O)OCH₂CH₂C≡CH | Z-1067 | —Me | —C(═O)OCH₂CH₂C≡CH | Z-1068 | —Et | —C(═O)OCH₂CH₂C≡CH |
| Z-1069 | —H | —C(═O)OCH₂C≡CCH₂CH₃ | Z-1070 | —Me | —C(═O)OCH₂C≡CCH₂CH₃ | Z-1071 | —Et | —C(═O)OCH₂C≡CCH₂CH₃ |
| Z-1072 | —H | —C(═O)OC(CH₃)₂C≡CH | Z-1073 | —Me | —C(═O)OC(CH₃)₂C≡CH | Z-1074 | —Et | —C(═O)OC(CH₃)₂C≡CH |
| Z-1075 | —H | —C(═O)OCH₂C≡CHCF₂H | Z-1076 | —Me | —C(═O)OCH₂C≡CHCF₂H | Z-1077 | —Et | —C(═O)OCH₂C≡CHCF₂H |
| Z-1078 | —H | —C(═O)OCH₂C≡CCF₃ | Z-1079 | —Me | —C(═O)OCH₂C≡CCF₃ | Z-1080 | —Et | —C(═O)OCH₂C≡CCF₃ |
| Z-1081 | —H | —C(═O)Ph | Z-1082 | —Me | —C(═O)Ph | Z-1083 | —Et | —C(═O)Ph |
| Z-1084 | —H | —C(═O)(2-Py) | Z-1085 | —Me | —C(═O)(2-Py) | Z-1086 | —Et | —C(═O)(2-Py) |
| Z-1087 | —H | —C(═O)(3-Py) | Z-1088 | —Me | —C(═O)(3-Py) | Z-1089 | —Et | —C(═O)(3-Py) |
| Z-1090 | —H | —C(═O)(4-Py) | Z-1091 | —Me | —C(═O)(4-Py) | Z-1092 | —Et | —C(═O)(4-Py) |
| Z-1093 | —H | —C(═O)CF₂Me | Z-1094 | —Me | —C(═O)CF₂Me | Z-1095 | —Et | —C(═O)CF₂Me |
| Z-1096 | —H | —C(═O)NMe2 | Z-1097 | —Me | —C(═O)NMe2 | Z-1098 | —Et | —C(═O)NMe2 |
| Z-1099 | —H | —C(═O)—(1-CF₃—c-Pr) | Z-1100 | —Me | —C(═O)—(1-CF₃—c-Pr) | Z-1101 | —Et | —C(═O)—(1-CF₃—c-Pr) |
| Z-1102 | —H | —C(═O)—(1-F—c-Pr) | Z-1103 | —Me | —C(═O)—(1-F—c-Pr) | Z-1104 | —Et | —C(═O)—(1-F—c-Pr) |
| Z-1105 | —H | —SO₂Me | Z-1106 | —Me | —SO₂Me | Z-1107 | —Et | —SO₂Me |
| Z-1108 | —H | —C(═O)CF═CH₂ | Z-1109 | —Me | —C(═O)CF═CH₂ | Z-1110 | —Et | —C(═O)CF═CH₂ |
| Z-1111 | —H | —C(═O)—(4-Cl—Ph) | Z-1112 | —Me | —C(═O)—(4-Cl—Ph) | Z-1113 | —Et | —C(═O)—(4-Cl—Ph) |
| Z-1114 | —H | —C(═O)—(3-Cl—Ph) | Z-1115 | —Me | —C(═O)—(3-Cl—Ph) | Z-1116 | —Et | —C(═O)—(3-Cl—Ph) |
| Z-1117 | —H | —C(═O)—(3-CF₃—Ph) | Z-1118 | —Me | —C(═O)—(3-CF₃—Ph) | Z-1119 | —Et | —C(═O)—(3-CF₃—Ph) |
| Z-1120 | —H | —C(═O)—(2-Cl—Ph) | Z-1121 | —Me | —C(═O)—(2-Cl—Ph) | Z-1122 | —Et | —C(═O)—(2-Cl—Ph) |
| Z-1123 | —H | —C(═O)—(2-CF₃—Ph) | Z-1124 | —Me | —C(═O)—(2-CF₃—Ph) | Z-1125 | —Et | —C(═O)—(2-CF₃—Ph) |
| Z-1126 | —H | —C(═O)—(4-CF₃—Ph) | Z-1127 | —Me | —C(═O)—(4-CF₃—Ph) | Z-1128 | —Et | —C(═O)—(4-CF₃—Ph) |
| Z-1129 | —H | —C(═O)—(3-F—Ph) | Z-1130 | —Me | —C(═O)—(3-F—Ph) | Z-1131 | —Et | —C(═O)—(3-F—Ph) |
| Z-1132 | —H | —C(═O)—(4-F—Ph) | Z-1133 | —Me | —C(═O)—(4-F—Ph) | Z-1134 | —Et | —C(═O)—(4-F—Ph) |
| Z-1135 | —H | —C(═O)—(2-F—Ph) | Z-1136 | —Me | —C(═O)—(2-F—Ph) | Z-1137 | —Et | —C(═O)—(2-F—Ph) |
| Z-1138 | —H | —C(═O)—(4-OCF₃—Ph) | Z-1139 | —Me | —C(═O)—(4-OCF₃—Ph) | Z-1140 | —Et | —C(═O)—(4-OCF₃—Ph) |
| Z-1141 | —H | —C(═O)—(6-Cl—3-Py) | Z-1142 | —Me | —C(═O)—(6-Cl—3-Py) | Z-1143 | —Et | —C(═O)—(6-Cl—3-Py) |
| Z-1144 | —H | —C(═O)—(6-CF3—2-Py) | Z-1145 | —Me | —C(═O)—(6-CF3—2-Py) | Z-1146 | —Et | —C(═O)—(6-CF3—2-Py) |
| Z-1147 | —H | —C(═O)—(1-CN—c-Pr) | Z-1148 | —Me | —C(═O)—(1-CN—c-Pr) | Z-1149 | —Et | —C(═O)—(1-CN—c-Pr) |
| Z-1150 | —H | —C(═O)—(3-Cl—2-Py) | Z-1151 | —Me | —C(═O)—(3-Cl—2-Py) | Z-1152 | —Et | —C(═O)—(3-Cl—2-Py) |
| Z-1153 | —H | —C(═O)—(2-Me—Ph) | Z-1154 | —Me | —C(═O)—(2-Me—Ph) | Z-1155 | —Et | —C(═O)—(2-Me—Ph) |
| Z-1156 | —H | —C(═O)—(3-Me—Ph) | Z-1157 | —Me | —C(═O)—(3-Me—Ph) | Z-1158 | —Et | —C(═O)—(3-Me—Ph) |
| Z-1159 | —H | —C(═O)—(4-Me—Ph) | Z-1160 | —Me | —C(═O)—(4-Me—Ph) | Z-1161 | —Et | —C(═O)—(4-Me—Ph) |
| Z-1162 | —H | —C(═O)—(2-MeO—Ph) | Z-1163 | —Me | —C(═O)—(2-MeO—Ph) | Z-1164 | —Et | —C(═O)—(2-MeO—Ph) |
| Z-1165 | —H | —C(═O)—(3-MeO—Ph) | Z-1166 | —Me | —C(═O)—(3-MeO—Ph) | Z-1167 | —Et | —C(═O)—(3-MeO—Ph) |
| Z-1168 | —H | —C(═O)—(4-MeO—Ph) | Z-1169 | —Me | —C(═O)—(4-MeO—Ph) | Z-1170 | —Et | —C(═O)—(4-MeO—Ph) |
| Z-1171 | —H | —C(═O)—(4-Cl—2-Py) | Z-1172 | —Me | —C(═O)—(4-Cl—2-Py) | Z-1173 | —Et | —C(═O)—(4-Cl—2-Py) |
| Z-1174 | —H | —C(═O)—(5-Cl—2-Py) | Z-1175 | —Me | —C(═O)—(5-Cl—2-Py) | Z-1176 | —Et | —C(═O)—(5-Cl—2-Py) |
| Z-1177 | —H | —C(═O)—(6-Cl—2-Py) | Z-1178 | —Me | —C(═O)—(6-Cl—2-Py) | Z-1179 | —Et | —C(═O)—(6-Cl—2-Py) |
| Z-1180 | —H | —C(═O)—(2-Cl—3-Py) | Z-1181 | —Me | —C(═O)—(2-Cl—3-Py) | Z-1182 | —Et | —C(═O)—(2-Cl—3-Py) |
| Z-1183 | —H | —C(═O)—(2-Cl—4-Py) | Z-1184 | —Me | —C(═O)—(2-Cl—4-Py) | Z-1185 | —Et | —C(═O)—(2-Cl—4-Py) |
| Z-1186 | —H | —C(═O)—(3-Cl—4-Py) | Z-1187 | —Me | —C(═O)—(3-Cl—4-Py) | Z-1188 | —Et | —C(═O)—(3-Cl—4-Py) |
| Z-1189 | —H | —C(═O)—(3,4-di-Me—Ph) | Z-1190 | —Me | —C(═O)—(3,4-di-Me—Ph) | Z-1191 | —Et | —C(═O)—(3,4-di-Me—Ph) |
| Z-1192 | —H | —C(═O)—(3,5-di-Me—Ph) | Z-1193 | —Me | —C(═O)—(3,5-di-Me—Ph) | Z-1194 | —Et | —C(═O)—(3,5-di-Me—Ph) |
| Z-1195 | —H | —C(═O)—(4-Cl—3-Py) | Z-1196 | —Me | —C(═O)—(4-Cl—3-Py) | Z-1197 | —Et | —C(═O)—(4-Cl—3-Py) |
| Z-1198 | —H | —C(═O)—(5-Cl—3-Py) | Z-1199 | —Me | —C(═O)—(5-Cl—3-Py) | Z-1200 | —Et | —C(═O)—(5-Cl—3-Py) |
| Z-1201 | —H | —C(═O)—(4-Pyrimidine) | Z-1202 | —Me | —C(═O)—(4-Pyrimidine) | Z-1203 | —Et | —C(═O)—(4-Pyrimidine) |
| Z-1204 | —H | —C(═O)—(2-Cl—4-Pyrimidine) | Z-1205 | —Me | —C(═O)—(2-Cl—4-Pyrimidine) | Z-1206 | —Et | —C(═O)—(2-Cl—4-Pyrimidine) |
| Z-1207 | —H | —C(═O)—(4-Et—Ph) | Z-1208 | —Me | —C(═O)—(4-Et—Ph) | Z-1209 | —Et | —C(═O)—(4-Et—Ph) |
| Z-1210 | —H | —C(═O)—(2-Me—4-Pyrimidine) | Z-1211 | —Me | —C(═O)—(2-Me—4-Pyrimidine) | Z-1212 | —Et | —C(═O)—(2-Me—4-Pyrimidine) |
| Z-1213 | —H | —C(═O)—(6-Me—4-Pyrimidine) | Z-1214 | —Me | —C(═O)—(6-Me—4-Pyrimidine) | Z-1215 | —Et | —C(═O)—(6-Me—4-Pyrimidine) |
| Z-1216 | —H | —C(═O)—(6-Me—2-Py) | Z-1217 | —Me | —C(═O)—(6-Me—2-Py) | Z-1218 | —Et | —C(═O)—(6-Me—2-Py) |
| Z-1219 | —H | —C(═O)—(2-CF₃—4-Pyrimidine) | Z-1220 | —Me | —C(═O)—(2-CF₃—4-Pyrimidine) | Z-1221 | —Et | —C(═O)—(2-CF₃—4-Pyrimidine) |
| Z-1222 | —H | —C(═O)—(3-Pyridazine) | Z-1223 | —Me | —C(═O)—(3-Pyridazine) | Z-1224 | —Et | —C(═O)—(3-Pyridazine) |
| Z-1225 | —H | —C(═O)—(1-Me—c-Pr) | Z-1226 | —Me | —C(═O)—(1-Me—c-Pr) | Z-1227 | —Et | —C(═O)—(1-Me—c-Pr) |
| Z-1228 | —H | —C(═O)—(1-CF₃—c-Bu) | Z-1229 | —Me | —C(═O)—(1-CF₃—c-Bu) | Z-1230 | —Et | —C(═O)—(1-CF₃—c-Bu) |
| Z-1231 | —H | —C(═O)—(2-Pyrimidine) | Z-1232 | —Me | —C(═O)—(2-Pyrimidine) | Z-1233 | —Et | —C(═O)—(2-Pyrimidine) |
| Z-1234 | —H | —C(═O)—(2-Pyrazine) | Z-1235 | —Me | —C(═O)—(2-Pyrazine) | Z-1236 | —Et | —C(═O)—(2-Pyrazine) |
| Z-1237 | —H | —C(═O)CH═CHOEt | Z-1238 | —Me | —C(═O)CH═CHOEt | Z-1239 | —Et | —C(═O)CH═CHOEt |
| Z-1240 | —H | —C(═O)CH₂CHCF₃CF₃ | Z-1241 | —Me | —C(═O)CH₂CHCF₃CF₃ | Z-1242 | —Et | —C(═O)CH₂CHCF₃CF₃ |
| Z-1243 | —H | —C(═O)CH₂—(c-Pr) | Z-1244 | —Me | —C(═O)CH₂—(c-Pr) | Z-1245 | —Et | —C(═O)CH₂—(c-Pr) |
| Z-1246 | —H | —C(═O)—(2,2,3,3-tetra-Me—c-Pr) | Z-1247 | —Me | —C(═O)—(2,2,3,3-tetra-Me—c-Pr) | Z-1248 | —Et | —C(═O)—(2,2,3,3-tetra-Me—c-Pr) |
| Z-1249 | —H | —C(═O)CMe₂CF₃ | Z-1250 | —Me | —C(═O)CMe₂CF₃ | Z-1251 | —Et | —C(═O)CMe₂CF₃ |
| Z-1252 | —H | —C(═O)CF₂Me | Z-1253 | —Me | —C(═O)CF₂Me | Z-1254 | —Et | —C(═O)CF₂Me |
| Z-1255 | —H | —C(═O)NMe₂ | Z-1256 | —Me | —C(═O)NMe₂ | Z-1257 | —Et | —C(═O)NMe₂ |
| Z-1258 | —H | —C(═O)—(1-CF₃—c-Pr) | Z-1259 | —Me | —C(═O)—(1-CF₃—c-Pr) | Z-1260 | —Et | —C(═O)—(1-CF₃—c-Pr) |
| Z-1261 | —H | —C(═O)—(1-F—c-Pr) | Z-1262 | —Me | —C(═O)—(1-F—c-Pr) | Z-1263 | —Et | —C(═O)—(1-F—c-Pr) |
| Z-1264 | —H | —SO₂Me | Z-1265 | —Me | —SO₂Me | Z-1266 | —Et | —SO₂Me |
| Z-1267 | —H | —C(═O)CF═CH₂ | Z-1268 | —Me | —C(═O)CF═CH₂ | Z-1269 | —Et | —C(═O)CF═CH₂ |
| Z-1270 | —H | —C(═O)—(4-Cl—Ph) | Z-1271 | —Me | —C(═O)—(4-Cl—Ph) | Z-1272 | —Et | —C(═O)—(4-Cl—Ph) |
| Z-1273 | —H | —C(═O)—(3-Cl—Ph) | Z-1274 | —Me | —C(═O)—(3-Cl—Ph) | Z-1275 | —Et | —C(═O)—(3-Cl—Ph) |
| Z-1276 | —H | —C(═O)—(3-CF₃—Ph) | Z-1277 | —Me | —C(═O)—(3-CF₃—Ph) | Z-1278 | —Et | —C(═O)—(3-CF₃—Ph) |
| Z-1279 | —H | —C(═O)—(2-Cl—Ph) | Z-1280 | —Me | —C(═O)—(2-Cl—Ph) | Z-1281 | —Et | —C(═O)—(2-Cl—Ph) |
| Z-1282 | —H | —C(═O)—(2-CF₃—Ph) | Z-1283 | —Me | —C(═O)—(2-CF₃—Ph) | Z-1284 | —Et | —C(═O)—(2-CF₃—Ph) |
| Z-1285 | —H | —C(═O)—(4-CF₃—Ph) | Z-1286 | —Me | —C(═O)—(4-CF₃—Ph) | Z-1287 | —Et | —C(═O)—(4-CF₃—Ph) |
| Z-1288 | —H | —C(═O)—(3-F—Ph) | Z-1289 | —Me | —C(═O)—(3-F—Ph) | Z-1290 | —Et | —C(═O)—(3-F—Ph) |
| Z-1291 | —H | —C(═O)—(4-F—Ph) | Z-1292 | —Me | —C(═O)—(4-F—Ph) | Z-1293 | —Et | —C(═O)—(4-F—Ph) |
| Z-1294 | —H | —C(═O)—(2-F—Ph) | Z-1295 | —Me | —C(═O)—(2-F—Ph) | Z-1296 | —Et | —C(═O)—(2-F—Ph) |
| Z-1297 | —H | —C(═O)—(4-OCF₃—Ph) | Z-1298 | —Me | —C(═O)—(4-OCF₃—Ph) | Z-1299 | —Et | —C(═O)—(4-OCF₃—Ph) |
| Z-1300 | —H | —C(═O)—(6-Cl—3-Py) | Z-1301 | —Me | —C(═O)—(6-Cl—3-Py) | Z-1302 | —Et | —C(═O)—(6-Cl—3-Py) |
| Z-1303 | —H | —C(═O)—(6-CF3—2-Py) | Z-1304 | —Me | —C(═O)—(6-CF3—2-Py) | Z-1305 | —Et | —C(═O)—(6-CF3—2-Py) |
| Z-1306 | —H | —C(═O)—(1-CN—c-Pr) | Z-1307 | —Me | —C(═O)—(1-CN—c-Pr) | Z-1308 | —Et | —C(═O)—(1-CN—c-Pr) |
| Z-1309 | —H | —C(═O)—(3-Cl—2-Py) | Z-1310 | —Me | —C(═O)—(3-Cl—2-Py) | Z-1311 | —Et | —C(═O)—(3-Cl—2-Py) |
| Z-1312 | —H | —C(═O)—(2-Me—Ph) | Z-1313 | —Me | —C(═O)—(2-Me—Ph) | Z-1314 | —Et | —C(═O)—(2-Me—Ph) |
| Z-1315 | —H | —C(═O)—(3-Me—Ph) | Z-1316 | —Me | —C(═O)—(3-Me—Ph) | Z-1317 | —Et | —C(═O)—(3-Me—Ph) |
| Z-1318 | —H | —C(═O)—(4-Me—Ph) | Z-1319 | —Me | —C(═O)—(4-Me—Ph) | Z-1320 | —Et | —C(═O)—(4-Me—Ph) |
| Z-1321 | —H | —C(═O)—(2-MeO—Ph) | Z-1322 | —Me | —C(═O)—(2-MeO—Ph) | Z-1323 | —Et | —C(═O)—(2-MeO—Ph) |
| Z-1324 | —H | —C(═O)—(3-MeO—Ph) | Z-1325 | —Me | —C(═O)—(3-MeO—Ph) | Z-1326 | —Et | —C(═O)—(3-MeO—Ph) |
| Z-1327 | —H | —C(═O)—(4-MeO—Ph) | Z-1328 | —Me | —C(═O)—(4-MeO—Ph) | Z-1329 | —Et | —C(═O)—(4-MeO—Ph) |
| Z-1330 | —H | —C(═O)—(4-Cl—2-Py) | Z-1331 | —Me | —C(═O)—(4-Cl—2-Py) | Z-1332 | —Et | —C(═O)—(4-Cl—2-Py) |
| Z-1333 | —H | —C(═O)—(5-Cl—2-Py) | Z-1334 | —Me | —C(═O)—(5-Cl—2-Py) | Z-1335 | —Et | —C(═O)—(5-Cl—2-Py) |
| Z-1336 | —H | —C(═O)—(6-Cl—2-Py) | Z-1337 | —Me | —C(═O)—(6-Cl—2-Py) | Z-1338 | —Et | —C(═O)—(6-Cl—2-Py) |
| Z-1339 | —H | —C(═O)—(2-Cl—3-Py) | Z-1340 | —Me | —C(═O)—(2-Cl—3-Py) | Z-1341 | —Et | —C(═O)—(2-Cl—3-Py) |
| Z-1342 | —H | —C(═O)—(2-Cl—4-Py) | Z-1343 | —Me | —C(═O)—(2-Cl—4-Py) | Z-1344 | —Et | —C(═O)—(2-Cl—4-Py) |
| Z-1345 | —H | —C(═O)—(3-Cl—4-Py) | Z-1346 | —Me | —C(═O)—(3-Cl—4-Py) | Z-1347 | —Et | —C(═O)—(3-Cl—4-Py) |
| Z-1348 | —H | —C(═O)—(3,4-di-Me—Ph) | Z-1349 | —Me | —C(═O)—(3,4-di-Me—Ph) | Z-1350 | —Et | —C(═O)—(3,4-di-Me—Ph) |
| Z-1351 | —H | —C(═O)—(3,5-di-Me—Ph) | Z-1352 | —Me | —C(═O)—(3,5-di-Me—Ph) | Z-1353 | —Et | —C(═O)—(3,5-di-Me—Ph) |
| Z-1354 | —H | —C(═O)—(4-Cl—3-Py) | Z-1355 | —Me | —C(═O)—(4-Cl—3-Py) | Z-1356 | —Et | —C(═O)—(4-Cl—3-Py) |
| Z-1357 | —H | —C(═O)—(5-Cl—3-Py) | Z-1358 | —Me | —C(═O)—(5-Cl—3-Py) | Z-1359 | —Et | —C(═O)—(5-Cl—3-Py) |
| Z-1360 | —H | —C(═O)—(4-Pyrimidine) | Z-1361 | —Me | —C(═O)—(4-Pyrimidine) | Z-1362 | —Et | —C(═O)—(4-Pyrimidine) |
| Z-1363 | —H | —C(═O)—(2-Cl—4-Pyrimidine) | Z-1364 | —Me | —C(═O)—(2-Cl—4-Pyrimidine) | Z-1365 | —Et | —C(═O)—(2-Cl—4-Pyrimidine) |
| Z-1366 | —H | —C(═O)—(4-Et—Ph) | Z-1367 | —Me | —C(═O)—(4-Et—Ph) | Z-1368 | —Et | —C(═O)—(4-Et—Ph) |
| Z-1369 | —H | —C(═O)—(2-Me—4-Pyrimidine) | Z-1370 | —Me | —C(═O)—(2-Me—4-Pyrimidine) | Z-1371 | —Et | —C(═O)—(2-Me—4-Pyrimidine) |
| Z-1372 | —H | —C(═O)—(6-Me—4-Pyrimidine) | Z-1373 | —Me | —C(═O)—(6-Me—4-Pyrimidine) | Z-1374 | —Et | —C(═O)—(6-Me—4-Pyrimidine) |
| Z-1375 | —H | —C(═O)—(6-Me—2-Py) | Z-1376 | —Me | —C(═O)—(6-Me—2-Py) | Z-1377 | —Et | —C(═O)—(6-Me—2-Py) |
| Z-1378 | —H | —C(═O)—(2-CF₃—4-Pyrimidine) | Z-1379 | —Me | —C(═O)—(2-CF₃—4-Pyrimidine) | Z-1380 | —Et | —C(═O)—(2-CF₃—4-Pyrimidine) |
| Z-1381 | —H | —C(═O)—(3-Pyridazine) | Z-1382 | —Me | —C(═O)—(3-Pyridazine) | Z-1383 | —Et | —C(═O)—(3-Pyridazine) |
| Z-1384 | —H | —C(═O)—(1-Me—c-Pr) | Z-1385 | —Me | —C(═O)—(1-Me—c-Pr) | Z-1386 | —Et | —C(═O)—(1-Me—c-Pr) |
| Z-1387 | —H | —C(═O)—(1-CF₃—c-Bu) | Z-1388 | —Me | —C(═O)—(1-CF₃—c-Bu) | Z-1389 | —Et | —C(═O)—(1-CF₃—c-Bu) |
| Z-1390 | —H | —C(═O)—(2-Pyrimidine) | Z-1391 | —Me | —C(═O)—(2-Pyrimidine) | Z-1392 | —Et | —C(═O)—(2-Pyrimidine) |
| Z-1393 | —H | —C(═O)—(2-Pyrazine) | Z-1394 | —Me | —C(═O)—(2-Pyrazine) | Z-1395 | —Et | —C(═O)—(2-Pyrazine) |
| Z-1396 | —H | —C(═O)CH═CHOEt | Z-1397 | —Me | —C(═O)CH═CHOEt | Z-1398 | —Et | —C(═O)CH═CHOEt |
| Z-1399 | —H | —C(═O)CH₂CHCF₃CF₃ | Z-1400 | —Me | —C(═O)CH₂CHCF₃CF₃ | Z-1401 | —Et | —C(═O)CH₂CHCF₃CF₃ |
| Z-1402 | —H | —C(═O)CH₂—(c-Pr) | Z-1403 | —Me | —C(═O)CH₂—(c-Pr) | Z-1404 | —Et | —C(═O)CH₂—(c-Pr) |
| Z-1405 | —H | —C(═O)—(2,2,3,3-tetra-Me—c-Pr) | Z-1406 | —Me | —C(═O)—(2,2,3,3-tetra-Me—c-Pr) | Z-1407 | —Et | —C(═O)—(2,2,3,3-tetra-Me—c-Pr) |
| Z-1408 | —H | —C(═O)CMe₂CF₃ | Z-1409 | —Me | —C(═O)CMe₂CF₃ | Z-1410 | —Et | —C(═O)CMe₂CF₃ |
| Z-1411 | —CH₂CF₃ | —H | Z-1412 | —Pr | —H | Z-1413 | —c-Pr | —H |
| Z-1414 | —CH₂CF₃ | —Me | Z-1415 | —Pr | —Me | Z-1416 | —c-Pr | —Me |
| Z-1417 | —CH₂CF₃ | —Et | Z-1418 | —Pr | —Et | Z-1419 | —c-Pr | —Et |
| Z-1420 | —CH₂CF₃ | —Pr | Z-1421 | —Pr | —Pr | Z-1422 | —c-Pr | —Pr |
| Z-1423 | —CH₂CF₃ | —i-Pr | Z-1424 | —Pr | —i-Pr | Z-1425 | —c-Pr | —i-Pr |
| Z-1426 | —CH₂CF₃ | —Bu | Z-1427 | —Pr | —Bu | Z-1428 | —c-Pr | —Bu |
| Z-1429 | —CH₂CF₃ | —sec-Bu | Z-1430 | —Pr | —sec-Bu | Z-1431 | —c-Pr | —sec-Bu |
| Z-1432 | —CH₂CF₃ | —I-Bu | Z-1433 | —Pr | —I-Bu | Z-1434 | —c-Pr | —I-Bu |
| Z-1435 | —CH₂CF₃ | —t-Bu | Z-1436 | —Pr | —t-Bu | Z-1437 | —c-Pr | —t-Bu |
| Z-1438 | —CH₂CF₃ | —Pent | Z-1439 | —Pr | —Pent | Z-1440 | —c-Pr | —Pent |
| Z-1441 | —CH₂CF₃ | —Hex | Z-1442 | —Pr | —Hex | Z-1443 | —c-Pr | —Hex |
| Z-1444 | —CH₂CF₃ | —CH(CH₃)CH₂CH₂CH₃ | Z-1445 | —Pr | —CH(CH₃)CH₂CH₂CH₃ | Z-1446 | —c-Pr | —CH(CH₃)CH₂CH₂CH₃ |
| Z-1447 | —CH₂CF₃ | —CH(CH₃)CH(CH₃)₂ | Z-1448 | —Pr | —CH(CH₃)CH(CH₃)₂ | Z-1449 | —c-Pr | —CH(CH₃)CH(CH₃)₂ |
| Z-1450 | —CH₂CF₃ | —C(CH₃)₂CH₂CH₃ | Z-1451 | —Pr | —C(CH₃)₂CH₂CH₃ | Z-1452 | —c-Pr | —C(CH₃)₂CH₂CH₃ |
| Z-1453 | —CH₂CF₃ | —CH(CH₂CH₃)₂ | Z-1454 | —Pr | —CH(CH₂CH₃)₂ | Z-1455 | —c-Pr | —CH(CH₂CH₃)₂ |
| Z-1456 | —CH₂CF₃ | —CH₂CH₂CH(CH₃)₂ | Z-1457 | —Pr | —CH₂CH₂CH(CH₃)₂ | Z-1458 | —c-Pr | —CH₂CH₂CH(CH₃)₂ |
| Z-1459 | —CH₂CF₃ | —CH₂CH₂CH₂CH(CH₃)₂ | Z-1460 | —Pr | —CH₂CH₂CH₂CH(CH₃)₂ | Z-1461 | —c-Pr | —CH₂CH₂CH₂CH(CH₃)₂ |
| Z-1462 | —CH₂CF₃ | —CH₂CH₂CH(CH₃)CH₂CH₃ | Z-1463 | —Pr | —CH₂CH₂CH(CH₃)CH₂CH₃ | Z-1464 | —c-Pr | —CH₂CH₂CH(CH₃)CH₂CH₃ |
| Z-1465 | —CH₂CF₃ | —CH₂CH(CH₃)CH₂CH₂CH₃ | Z-1466 | —Pr | —CH₂CH(CH₃)CH₂CH₂CH₃ | Z-1467 | —c-Pr | —CH₂CH(CH₃)CH₂CH₂CH₃ |
| Z-1468 | —CH₂CF₃ | —CH(CH₃)CH₂CH₂CH₂CH₃ | Z-1469 | —Pr | —CH(CH₃)CH₂CH₂CH₂CH₃ | Z-1470 | —c-Pr | —CH(CH₃)CH₂CH₂CH₂CH₃ |
| Z-1471 | —CH₂CF₃ | —CH(CH3)CH₂CH(CH₃)₂ | Z-1472 | —Pr | —CH(CH3)CH₂CH(CH₃)₂ | Z-1473 | —c-Pr | —CH(CH3)CH₂CH(CH₃)₂ |
| Z-1474 | —CH₂CF₃ | —CH(CH₃)CH(CH₃)CH₂CH₃ | Z-1475 | —Pr | —CH(CH₃)CH(CH₃)CH₂CH₃ | Z-1476 | —c-Pr | —CH(CH₃)CH(CH₃)CH₂CH₃ |
| 2-1477 | —CH₂CF₃ | —C(CH₃)₂CH₂CH₂CH₃ | Z-1478 | —Pr | —C(CH₃)₂CH₂CH₂CH₃ | Z-1479 | —c-Pr | —C(CH₃)₂CH₂CH₂CH₃ |
| Z-1480 | —CH₂CF₃ | —CH(CH₃)C(CH₃)₃ | Z-1481 | —Pr | —CH(CH₃)C(CH₃)₃ | Z-1482 | —c-Pr | —CH(CH₃)C(CH₃)₃ |
| Z-1483 | —CH₂CF₃ | —C(CH₃)₂CH(CH₃)₂ | Z-1484 | —Pr | —C(CH₃)₂CH(CH₃)₂ | Z-1485 | —c-Pr | —C(CH₃)₂CH(CH₃)₂ |
| Z-1486 | —CH₂CF₃ | —CH₂CH₂C(CH₃)₃ | Z-1487 | —Pr | —CH₂CH₂C(CH₃)₃ | Z-1488 | —c-Pr | —CH₂CH₂C(CH₃)₃ |
| Z-1489 | —CH₂CF₃ | —CH₂CH(CH₃)CH(CH₃)₂ | Z-1490 | —Pr | —CH₂CH(CH₃)CH(CH₃)₂ | Z-1491 | —c-Pr | —CH₂CH(CH₃)CH(CH₃)₂ |
| Z-1492 | —CH₂CF₃ | —CH₂C(CH₃)₂CH₂CH₃ | Z-1493 | —Pr | —CH₂C(CH₃)₂CH₂CH₃ | Z-1494 | —c-Pr | —CH₂C(CH₃)₂CH₂CH₃ |
| Z-1495 | —CH₂CF₃ | —CFH₂ | Z-1496 | —Pr | —CFH₂ | Z-1497 | —c-Pr | —CFH₂ |
| Z-1498 | —CH₂CF₃ | —CF₂H | Z-1499 | —Pr | —CF₂H | Z-1500 | —c-Pr | —CF₂H |
| Z-1501 | —CH₂CF₃ | —CF₃ | Z-1502 | —Pr | —CF₃ | Z-1503 | —c-Pr | —CF₃ |
| Z-1504 | —CH₂CF₃ | —CH₂Cl | Z-1505 | —Pr | —CH₂Cl | Z-1506 | —c-Pr | —CH₂Cl |
| Z-1507 | —CH₂CF₃ | —CHCl₂ | Z-1508 | —Pr | —CHCl₂ | Z-1509 | —c-Pr | —CHCl₂ |
| Z-1510 | —CH₂CF₃ | —CCl₃ | Z-1511 | —Pr | —CCl₃ | Z-1512 | —c-Pr | —CCl₃ |
| Z-1513 | —CH₂CF₃ | —CF₂Cl | Z-1514 | —Pr | —CF₂Cl | Z-1515 | —c-Pr | —CF₂Cl |
| Z-1516 | —CH₂CF₃ | —CCl₂F | Z-1517 | —Pr | —CCl₂F | Z-1518 | —c-Pr | —CCl₂F |
| Z-1519 | —CH₂CF₃ | —CH₂Br | Z-1520 | —Pr | —CH₂Br | Z-1521 | —c-Pr | —CH₂Br |
| Z-1522 | —CH₂CF₃ | —CHBr₂ | Z-1523 | —Pr | —CHBr₂ | Z-1524 | —c-Pr | —CHBr₂ |
| Z-1525 | —CH₂CF₃ | —CBr₃ | Z-1526 | —Pr | —CBr₃ | Z-1527 | —c-Pr | —CBr₃ |
| Z-1528 | —CH₂CF₃ | —CH₂l | Z-1529 | —Pr | —CH₂l | Z-1530 | —c-Pr | —CH₂l |
| Z-1531 | —CH₂CF₃ | —CHl₂ | Z-1532 | —Pr | —CHl₂ | Z-1533 | —c-Pr | —CHl₂ |
| Z-1534 | —CH₂CF₃ | —CH₂CF₂H | Z-1535 | —Pr | —CH₂CF₂H | Z-1536 | —c-Pr | —CH₂CF₂H |
| Z-1537 | —CH₂CF₃ | —CH₂CF₃ | Z-1538 | —Pr | —CH₂CF₃ | Z-1539 | —c-Pr | —CH₂CF₃ |
| Z-1540 | —CH₂CF₃ | —CF₂CH₃ | Z-1541 | —Pr | —CF₂CH₃ | Z-1542 | —c-Pr | —CF₂CH₃ |
| Z-1543 | —CH₂CF₃ | —CH₂CH₂CF₂H | Z-1544 | —Pr | —CH₂CH₂CF₂H | Z-1545 | —c-Pr | —CH₂CH₂CF₂H |
| Z-1546 | —CH₂CF₃ | —CH₂CH₂CF₃ | Z-1547 | —Pr | —CH₂CH₂CF₃ | Z-1548 | —c-Pr | —CH₂CH₂CF₃ |
| Z-1549 | —CH₂CF₃ | —CH₂CH₂CH₂CF₂H | Z-1550 | —Pr | —CH₂CH₂CH₂CF₂H | Z-1551 | —c-Pr | —CH₂CH₂CH₂CF₂H |
| Z-1552 | —CH₂CF₃ | —CH₂CH₂CH₂CF₃ | Z-1553 | —Pr | —CH₂CH₂CH₂CF₃ | Z-1554 | —c-Pr | —CH₂CH₂CH₂CF₃ |
| Z-1555 | —CH₂CF₃ | —CF₂CF₂H | Z-1556 | —Pr | —CF₂CF₂H | Z-1557 | —c-Pr | —CF₂CF₂H |
| Z-1558 | —CH₂CF₃ | —CF₂CF₂Cl | Z-1559 | —Pr | —CF₂CF₂Cl | Z-1560 | —c-Pr | —CF₂CF₂Cl |
| Z-1561 | —CH₂CF₃ | —CF₂CF₃ | Z-1562 | —Pr | —CF₂CF₃ | Z-1563 | —c-Pr | —CF₂CF₃ |
| Z-1564 | —CH₂CF₃ | —CFHCF₃ | Z-1565 | —Pr | —CFHCF₃ | Z-1566 | —c-Pr | —CFHCF₃ |
| Z-1567 | —CH₂CF₃ | —CH₂CF₂CF₂H | Z-1568 | —Pr | —CH₂CF₂CF₂H | Z-1569 | —c-Pr | —CH₂CF₂CF₂H |
| Z-1570 | —CH₂CF₃ | —CH₂CF₂CF₃ | Z-1571 | —Pr | —CH₂CF₂CF₃ | Z-1572 | —c-Pr | —CH₂CF₂CF₃ |
| Z-1573 | —CH₂CF₃ | —CF₂CF₂CF₃ | Z-1574 | —Pr | —CF₂CF₂CF₃ | Z-1575 | —c-Pr | —CF₂CF₂CF₃ |
| Z-1576 | —CH₂CF₃ | —CH₂CF₂CF₂CF₃ | Z-1577 | —Pr | —CH₂CF₂CF₂CF₃ | Z-1578 | —c-Pr | —CH₂CF₂CF₂CF₃ |
| Z-1579 | —CH₂CF₃ | —CF₂CF₂CF₂CF₃ | Z-1580 | —Pr | —CF₂CF₂CF₂CF₃ | Z-1581 | —c-Pr | —CF₂CF₂CF₂CF₃ |
| Z-1582 | —CH₂CF₃ | —CH₂CF₂CF₂CF₂CF₃ | Z-1583 | —Pr | —CH₂CF₂CF₂CF₂CF₃ | Z-1584 | —c-Pr | —CH₂CF₂CF₂CF₂CF₃ |
| Z-1585 | —CH₂CF₃ | c-Pr | Z-1586 | —Pr | c-Pr | Z-1587 | —c-Pr | c-Pr |
| Z-1588 | —CH₂CF₃ | c-Bu | Z-1589 | —Pr | c-Bu | Z-1590 | —c-Pr | c-Bu |
| Z-1591 | —CH₂CF₃ | c-Pent | Z-1592 | —Pr | c-Pent | Z-1593 | —c-Pr | c-Pent |
| Z-1594 | —CH₂CF₃ | c-Hex | Z-1595 | —Pr | c-Hex | Z-1596 | —c-Pr | c-Hex |
| Z-1597 | —CH₂CF₃ | c-Hept | Z-1598 | —Pr | c-Hept | Z-1599 | —c-Pr | c-Hept |
| Z-1600 | —CH₂CF₃ | c-Oct | Z-1601 | —Pr | c-Oct | Z-1602 | —c-Pr | c-Oct |
| Z-1603 | —CH₂CF₃ | —CH═CH₂ | Z-1604 | —Pr | —CH═CH₂ | Z-1605 | —c-Pr | —CH═CH₂ |
| Z-1606 | —CH₂CF₃ | —CH₂CH═CH₂ | Z-1607 | —Pr | —CH₂CH═CH₂ | Z-1608 | —c-Pr | —CH₂CH═CH₂ |
| Z-1609 | —CH₂CF₃ | —CH═CHCH₃ | Z-1610 | —Pr | —CH═CHCH₃ | Z-1611 | —c-Pr | —CH═CHCH₃ |
| Z-1612 | —CH₂CF₃ | —CH₂C(CH₃)═CH₂ | Z-1613 | —Pr | —CH₂C(CH₃)═CH₂ | Z-1614 | —c-Pr | —CH₂C(CH₃)═CH₂ |
| Z-1615 | —CH₂CF₃ | —CH₂CH₂CH═CH₂ | Z-1616 | —Pr | —CH₂CH₂CH═CH₂ | Z-1617 | —c-Pr | —CH₂CH₂CH═CH₂ |
| Z-1618 | —CH₂CF₃ | —CH₂CH═CHCH₃ | Z-1619 | —Pr | —CH₂CH═CHCH₃ | Z-1620 | —c-Pr | —CH₂CH═CHCH₃ |
| Z-1621 | —CH₂CF₃ | —CH═CHCH₂CH₃ | Z-1622 | —Pr | —CH═CHCH₂CH₃ | Z-1623 | —c-Pr | —CH═CHCH₂CH₃ |
| Z-1624 | —CH₂CF₃ | —CH₂CH═C(CH₃)₂ | Z-1625 | —Pr | —CH₂CH═C(CH₃)₂ | Z-1626 | —c-Pr | —CH₂CH═C(CH₃)₂ |
| Z-1627 | —CH₂CF₃ | —CH₂CH₂CH═C(CH₃)₂ | Z-1628 | —Pr | —CH₂CH₂CH═C(CH₃)₂ | Z-1629 | —c-Pr | —CH₂CH₂CH═C(CH₃)₂ |
| Z-1630 | —CH₂CF₃ | —CH═CFH | Z-1631 | —Pr | —CH═CFH | Z-1632 | —c-Pr | —CH═CFH |
| Z-1633 | —CH₂CF₃ | —CH═CF₂ | Z-1634 | —Pr | —CH═CF₂ | Z-1635 | —c-Pr | —CH═CF₂ |
| Z-1636 | —CH₂CF₃ | —CH═CCl₂ | Z-1637 | —Pr | —CH═CCl₂ | Z-1638 | —c-Pr | —CH═CCl₂ |
| Z-1639 | —CH₂CF₃ | —CH₂CH═CFH | Z-1640 | —Pr | —CH₂CH═CFH | Z-1641 | —c-Pr | —CH₂CH═CFH |
| Z-1642 | —CH₂CF₃ | —CH₂CH═CF₂ | Z-1643 | —Pr | —CH₂CH═CF₂ | Z-1644 | —c-Pr | —CH₂CH═CF₂ |
| Z-1645 | —CH₂CF₃ | —CH₂CH═CCl₂ | Z-1646 | —Pr | —CH₂CH═CCl₂ | Z-1647 | —c-Pr | —CH₂CH═CCl₂ |
| Z-1648 | —CH₂CF₃ | —CH₂CH₂CH═CF₂ | Z-1649 | —Pr | —CH₂CH₂CH═CF₂ | Z-1650 | —c-Pr | —CH₂CH₂CH═CF₂ |
| Z-1651 | —CH₂CF₃ | —CH₂CH₂CH₂CH═CF₂ | Z-1652 | —Pr | —CH₂CH₂CH₂CH═CF₂ | Z-1653 | —c-Pr | —CH₂CH₂CH₂CH═CF₂ |
| Z-1654 | —CH₂CF₃ | —CH₂CH₂CH₂CH₂CH═CF₂ | Z-1655 | —Pr | —CH₂CH₂CH₂CH₂CH═CF₂ | Z-1656 | —c-Pr | —CH₂CH₂CH₂CH₂CH═CF₂ |
| Z-1657 | —CH₂CF₃ | —C≡CH | Z-1658 | —Pr | —C≡CH | Z-1659 | —c-Pr | —C≡CH |
| Z-1660 | —CH₂CF₃ | —C≡CCH₃ | Z-1661 | —Pr | —C≡CCH₃ | Z-1662 | —c-Pr | —C≡CCH₃ |
| Z-1663 | —CH₂CF₃ | —CH₂C≡CH | Z-1664 | —Pr | —CH₂C≡CH | Z-1665 | —c-Pr | —CH₂C≡CH |
| Z-1666 | —CH₂CF₃ | —C≡CCH₂CH₃ | Z-1667 | —Pr | —C≡CCH₂CH₃ | Z-1668 | —c-Pr | —C≡CCH₂CH₃ |
| Z-1669 | —CH₂CF₃ | —CH₂C≡CCH₃ | Z-1670 | —Pr | —CH₂C≡CCH₃ | Z-1671 | —c-Pr | —CH₂C≡CCH₃ |
| Z-1672 | —CH₂CF₃ | —CH₂CH₂C≡CH | Z-1673 | —Pr | —CH₂CH₂C≡CH | Z-1674 | —c-Pr | —CH₂CH₂C≡CH |
| Z-1675 | —CH₂CF₃ | —C≡CCH₂CH₂CH₃ | Z-1676 | —Pr | —C≡CCH₂CH₂CH₃ | Z-1677 | —c-Pr | —C≡CCH₂CH₂CH₃ |
| Z-1678 | —CH₂CF₃ | —CH₂C≡CCH₂CH₃ | Z-1679 | —Pr | —CH₂C≡CCH₂CH₃ | Z-1680 | —c-Pr | —CH₂C≡CCH₂CH₃ |
| Z-1681 | —CH₂CF₃ | —C(CH₃)₂C≡CH | Z-1682 | —Pr | —C(CH₃)₂C≡CH | Z-1683 | —c-Pr | —C(CH₃)₂C≡CH |
| Z-1684 | —CH₂CF₃ | —C≡CF | Z-1685 | —Pr | —C≡CF | Z-1686 | —c-Pr | —C≡CF |
| Z-1687 | —CH₂CF₃ | —C≡CCF₂H | 2-1688 | —Pr | —C≡CCF₂H | Z-1689 | —c-Pr | —C≡CCF₂H |
| Z-1690 | —CH₂CF₃ | —C≡CCF₃ | Z-1691 | —Pr | —C≡CCF₃ | Z-1692 | —c-Pr | —C≡CCF₃ |
| Z-1693 | —CH₂CF₃ | —C≡CCH₂CF₂H | Z-1694 | —Pr | —C≡CCH₂CF₂H | Z-1695 | —c-Pr | —C≡CCH₂CF₂H |
| Z-1696 | —CH₂CF₃ | —C≡CCH₂CF₃ | Z-1697 | —Pr | —C≡CCH₂CF₃ | Z-1698 | —c-Pr | —C≡CCH₂CF₃ |
| Z-1699 | —CH₂CF₃ | —CH₂C≡CHCF₂H | Z-1700 | —Pr | —CH₂C≡CHCF₂H | Z-1701 | —c-Pr | —CH₂C≡CHCF₂H |
| Z-1702 | —CH₂CF₃ | —CH₂C≡CCF₃ | Z-1703 | —Pr | —CH₂C≡CCF₃ | Z-1704 | —c-Pr | —CH₂C≡CCF₃ |
| Z-1705 | —CH₂CF₃ | —C(═O)NH₂ | Z-1706 | —Pr | —C(═O)NH₂ | Z-1707 | —c-Pr | —C(═O)NH₂ |
| Z-1708 | —CH₂CF₃ | —C(═O)NHMe | Z-1709 | —Pr | —C(═O)NHMe | Z-1710 | —c-Pr | —C(═O)NHMe |
| Z-1711 | —CH₂CF₃ | —C(═O)NHEt | Z-1712 | —Pr | —C(═O)NHEt | Z-1713 | —c-Pr | —C(═O)NHEt |
| Z-1714 | —CH₂CF₃ | —C(═O)NHPr | Z-1715 | —Pr | —C(═O)NHPr | Z-1716 | —c-Pr | —C(═O)NHPr |
| Z-1717 | —CH₂CF₃ | —C(═O)NH—i-Pr | Z-1718 | —Pr | —C(═O)NH—i-Pr | Z-1719 | —c-Pr | —C(═O)NH—i-Pr |
| Z-1720 | —CH₂CF₃ | —C(═O)NHBu | Z-1721 | —Pr | —C(═O)NHBu | Z-1722 | —c-Pr | —C(═O)NHBu |
| Z-1723 | —CH₂CF₃ | —C(═O)NH—sec-Bu | Z-1724 | —Pr | —C(═O)NH—sec-Bu | Z-1725 | —c-Pr | —C(═O)NH—sec-Bu |
| Z-1726 | —CH₂CF₃ | —C(═O)NH—i-Bu | Z-1727 | —Pr | —C(═O)NH—i-Bu | Z-1728 | —c-Pr | —C(═O)NH—i-Bu |
| Z-1729 | —CH₂CF₃ | —C(═O)NH—t-Bu | Z-1730 | —Pr | —C(═O)NH—t-Bu | Z-1731 | —c-Pr | —C(═O)NH—t-Bu |
| Z-1732 | —CH₂CF₃ | —C(═O)NHPent | Z-1733 | —Pr | —C(═O)NHPent | Z-1734 | —c-Pr | —C(═O)NHPent |
| Z-1735 | —CH₂CF₃ | —C(═O)NHHex | Z-1736 | —Pr | —C(═O)NHHex | Z-1737 | —c-Pr | —C(═O)NHHex |
| Z-1738 | —CH₂CF₃ | —C(═O)NHCH₂C≡N | Z-1739 | —Pr | —C(═O)NHCH₂C≡N | Z-1740 | —c-Pr | —C(═O)NHCH₂C≡N |
| Z-1741 | —CH₂CF₃ | —C(═O)NHCH₂—c-Pr | Z-1742 | —Pr | —C(═O)NHCH₂—c-Pr | Z-1743 | —c-Pr | —C(═O)NHCH₂—c-Pr |
| Z-1744 | —CH₂CF₃ | —C(═O)NHCH₂OMe | Z-1745 | —Pr | —C(═O)NHCH₂OMe | Z-1746 | —c-Pr | —C(═O)NHCH₂OMe |
| Z-1747 | —CH₂CF₃ | —C(═O)NHCH₂CH₂OMe | Z-1748 | —Pr | —C(═O)NHCH₂CH₂OMe | Z-1749 | —c-Pr | —C(═O)NHCH₂CH₂OMe |
| Z-1750 | —CH₂CF₃ | —C(═O)NHCFH₂ | Z-1751 | —Pr | —C(═O)NHCFH₂ | Z-1752 | —c-Pr | —C(═O)NHCFH₂ |
| Z-1753 | —CH₂CF₃ | —C(═O)NHCF₂H | Z-1754 | —Pr | —C(═O)NHCF₂H | Z-1755 | —c-Pr | —C(═O)NHCF₂H |
| Z-1756 | —CH₂CF₃ | —C(═O)NHCF₃ | Z-1757 | —Pr | —C(═O)NHCF₃ | Z-1758 | —c-Pr | —C(═O)NHCF₃ |
| Z-1759 | —CH₂CF₃ | —C(═O)NHCH₂Cl | Z-1760 | —Pr | —C(═O)NHCH₂Cl | Z-1761 | —c-Pr | —C(═O)NHCH₂Cl |
| Z-1762 | —CH₂CF₃ | —C(═O)NHCHCl₂ | Z-1763 | —Pr | —C(═O)NHCHCl₂ | Z-1764 | —c-Pr | —C(═O)NHCHCl₂ |
| Z-1765 | —CH₂CF₃ | —C(═O)NHCCl₃ | Z-1766 | —Pr | —C(═O)NHCCl₃ | Z-1767 | —c-Pr | —C(═O)NHCCl₃ |
| Z-1768 | —CH₂CF₃ | —C(═O)NHCH₂Br | Z-1769 | —Pr | —C(═O)NHCH₂Br | Z-1770 | —c-Pr | —C(═O)NHCH₂Br |
| Z-1771 | —CH₂CF₃ | —C(═O)NHCHBr₂ | Z-1772 | —Pr | —C(═O)NHCHBr₂ | Z-1773 | —c-Pr | —C(═O)NHCHBr₂ |
| Z-1774 | —CH₂CF₃ | —C(═O)NHCBr₃ | Z-1775 | —Pr | —C(═O)NHCBr₃ | Z-1776 | —c-Pr | —C(═O)NHCBr₃ |
| Z-1777 | —CH₂CF₃ | —C(═O)NHCH₂l | Z-1778 | —Pr | —C(═O)NHCH₂l | Z-1779 | —c-Pr | —C(═O)NHCH₂l |
| Z-1780 | —CH₂CF₃ | —C(═O)NHCHl₂ | Z-1781 | —Pr | —C(═O)NHCHl₂ | Z-1782 | —c-Pr | —C(═O)NHCHl₂ |
| Z-1783 | —CH₂CF₃ | —C(═O)NHCH₂CF₂H | Z-1784 | —Pr | —C(═O)NHCH₂CF₂H | Z-1785 | —c-Pr | —C(═O)NHCH₂CF₂H |
| Z-1786 | —CH₂CF₃ | —C(═O)NHCH₂CF₃ | Z-1787 | —Pr | —C(═O)NHCH₂CF₃ | Z-1788 | —c-Pr | —C(═O)NHCH₂CF₃ |
| Z-1789 | —CH₂CF₃ | —C(═O)NHCH₂CH₂CF₂H | Z-1790 | —Pr | —C(═O)NHCH₂CH₂CF₂H | Z-1791 | —c-Pr | —C(═O)NHCH₂CH₂CF₂H |
| Z-1792 | —CH₂CF₃ | —C(═O)NHCH₂CH₂CF₃ | Z-1793 | —Pr | —C(═O)NHCH₂CH₂CF₃ | Z-1794 | —c-Pr | —C(═O)NHCH₂CH₂CF₃ |
| Z-1795 | —CH₂CF₃ | —C(═O)NHCH₂CH₂CH₂CF₂H | Z-1796 | —Pr | —C(═O)NHCH₂CH₂CH₂CF₂H | Z-1797 | —c-Pr | —C(═O)NHCH₂CH₂CH₂CF₂H |
| Z-1798 | —CH₂CF₃ | —C(═O)NHCH₂CH₂CH₂CF₃ | Z-1799 | —Pr | —C(═O)NHCH₂CH₂CH₂CF₃ | Z-1800 | —c-Pr | —C(═O)NHCH₂CH₂CH₂CF₃ |
| Z-1801 | —CH₂CF₃ | —C(═O)NHCF₂CF₂H | Z-1802 | —Pr | —C(═O)NHCF₂CF₂H | Z-1803 | —c-Pr | —C(═O)NHCF₂CF₂H |
| Z-1804 | —CH₂CF₃ | —C(═O)NHCF₂CF₃ | Z-1805 | —Pr | —C(═O)NHCF₂CF₃ | Z-1806 | —c-Pr | —C(═O)NHCF₂CF₃ |
| Z-1807 | —CH₂CF₃ | —C(═O)NHCFHCF₃ | Z-1808 | —Pr | —C(═O)NHCFHCF₃ | Z-1809 | —c-Pr | —C(═O)NHCFHCF₃ |
| Z-1810 | —CH₂CF₃ | —C(═O)NHCH₂CF₂CF₂H | Z-1811 | —Pr | —C(═O)NHCH₂CF₂CF₂H | Z-1812 | —c-Pr | —C(═O)NHCH₂CF₂CF₂H |
| Z-1813 | —CH₂CF₃ | —C(═O)NHCH₂CF₂CF₃ | Z-1814 | —Pr | —C(═O)NHCH₂CF₂CF₃ | Z-1815 | —c-Pr | —C(═O)NHCH₂CF₂CF₃ |
| Z-1816 | —CH₂CF₃ | —C(═O)NHCF₂CF₂CF₃ | Z-1817 | —Pr | —C(═O)NHCF₂CF₂CF₃ | Z-1818 | —c-Pr | —C(═O)NHCF₂CF₂CF₃ |
| Z-1819 | —CH₂CF₃ | —C(═O)NHCH₂CF₂CF₂CF₃ | Z-1820 | —Pr | —C(═O)NHCH₂CF₂CF₂CF₃ | Z-1821 | —c-Pr | —C(═O)NHCH₂CF₂CF₂CF₃ |
| Z-1822 | —CH₂CF₃ | —C(═O)NHCF₂CF₂CF₂CF₃ | Z-1823 | —Pr | —C(═O)NHCF₂CF₂CF₂CF₃ | Z-1824 | —c-Pr | —C(═O)NHCF₂CF₂CF₂CF₃ |
| Z-1825 | —CH₂CF₃ | —C(═O)NHCH₂CF₂CF₂CF₂CF₃ | Z-1826 | —Pr | —C(═O)NHCH₂CF₂CF₂CF₂CF₃ | Z-1827 | —c-Pr | —C(═O)NHCH₂CF₂CF₂CF₂CF₃ |
| Z-1828 | —CH₂CF₃ | —C(═O)NH—c-Pr | Z-1829 | —Pr | —C(═O)NH—c-Pr | Z-1830 | —c-Pr | —C(═O)NH—c-Pr |
| Z-1831 | —CH₂CF₃ | —C(═O)NH—c-Bu | Z-1832 | —Pr | —C(═O)NH—c-Bu | Z-1833 | —c-Pr | —C(═O)NH—c-Bu |
| Z-1834 | —CH₂CF₃ | —C(═O)NH—c-Pent | Z-1835 | —Pr | —C(═O)NH—c-Pent | Z-1836 | —c-Pr | —C(═O)NH—c-Pent |
| Z-1837 | —CH₂CF₃ | —C(═O)NH—c-Hex | Z-1838 | —Pr | —C(═O)NH—c-Hex | Z-1839 | —c-Pr | —C(═O)NH—c-Hex |
| Z-1840 | —CH₂CF₃ | —C(═O)NH—c-Hept | Z-1841 | —Pr | —C(═O)NH—c-Hept | Z-1842 | —c-Pr | —C(═O)NH—c-Hept |
| Z-1843 | —CH₂CF₃ | —C(═O)NH—c-Oct | Z-1844 | —Pr | —C(═O)NH—c-Oct | Z-1845 | —c-Pr | —C(═O)NH—c-Oct |
| Z-1846 | —CH₂CF₃ | —C(═O)NHCH₂CH═CH₂ | Z-1847 | —Pr | —C(═O)NHCH₂CH═CH₂ | Z-1848 | —c-Pr | —C(═O)NHCH₂CH═CH₂ |
| Z-1849 | —CH₂CF₃ | —C(═O)NHCH₂C(CH₃)═CH₂ | Z-1850 | —Pr | —C(═O)NHCH₂C(CH₃)═CH₂ | Z-1851 | —c-Pr | —C(═O)NHCH₂C(CH₃)═CH₂ |
| Z-1852 | —CH₂CF₃ | —C(═O)NHCH₂CH₂CH═CH₂ | Z-1853 | —Pr | —C(═O)NHCH₂CH₂CH═CH₂ | Z-1854 | —c-Pr | —C(═O)NHCH₂CH₂CH═CH₂ |
| Z-1855 | —CH₂CF₃ | —C(═O)NHCH₂CH═CHCH₃ | Z-1856 | —Pr | —C(═O)NHCH₂CH═CHCH₃ | Z-1857 | —c-Pr | —C(═O)NHCH₂CH═CHCH₃ |
| Z-1858 | —CH₂CF₃ | —C(═O)NHCH₂CH═C(CH₃)₂ | Z-1859 | —Pr | —C(═O)NHCH₂CH═C(CH₃)₂ | Z-1860 | —c-Pr | —C(═O)NHCH₂CH═C(CH₃)₂ |
| Z-1861 | —CH₂CF₃ | —C(═O)NHCH₂CH₂CH═C(CH₃)₂ | Z-1862 | —Pr | —C(═O)NHCH₂CH₂CH═C(CH₃)₂ | Z-1863 | —c-Pr | —C(═O)NHCH₂CH₂CH═C(CH₃)₂ |
| Z-1864 | —CH₂CF₃ | —C(═O)NHCH₂CH═CFH | Z-1865 | —Pr | —C(═O)NHCH₂CH═CFH | Z-1866 | —c-Pr | —C(═O)NHCH₂CH═CFH |
| Z-1867 | —CH₂CF₃ | —C(═O)NHCH₂CH═CF₂ | Z-1868 | —Pr | —C(═O)NHCH₂CH═CF₂ | Z-1869 | —c-Pr | —C(═O)NHCH₂CH═CF₂ |
| Z-1870 | —CH₂CF₃ | —C(═O)NHCH₂CH═CCl₂ | Z-1871 | —Pr | —C(═O)NHCH₂CH═CCl₂ | Z-1872 | —c-Pr | —C(═O)NHCH₂CH═CCl₂ |
| Z-1873 | —CH₂CF₃ | —C(═O)NHCH₂CH₂CH═CF₂ | Z-1874 | —Pr | —C(═O)NHCH₂CH₂CH═CF₂ | Z-1875 | —c-Pr | —C(═O)NHCH₂CH₂CH═CF₂ |
| Z-1876 | —CH₂CF₃ | —C(═O)NHCH₂CH₂CH₂CH═CF₂ | Z-1877 | —Pr | —C(═O)NHCH₂CH₂CH₂CH═CF₂ | Z-1878 | —c-Pr | —C(═O)NHCH₂CH₂CH₂CH═CF₂ |
| Z-1879 | —CH₂CF₃ | —C(═O)NHCH₂CH₂CH₂CH₂CH═CF₂ | Z-1880 | —Pr | —C(═O)NHCH₂CH₂CH₂CH₂CH═CF₂ | Z-1881 | —c-Pr | —C(═O)NHCH₂CH₂CH₂CH₂CH═CF₂ |
| Z-1882 | —CH₂CF₃ | —C(═O)NHCH₂C≡CH | Z-1883 | —Pr | —C(═O)NHCH₂C≡CH | Z-1884 | —c-Pr | —C(═O)NHCH₂C≡CH |
| Z-1885 | —CH₂CF₃ | —C(═O)NHCH₂C≡CCH₃ | Z-1886 | —Pr | —C(═O)NHCH₂C≡CCH₃ | Z-1887 | —c-Pr | —C(═O)NHCH₂C≡CCH₃ |
| Z-1888 | —CH₂CF₃ | —C(═O)NHCH₂CH₂C≡CH | Z-1889 | —Pr | —C(═O)NHCH₂CH₂C≡CH | Z-1890 | —c-Pr | —C(═O)NHCH₂CH₂C≡CH |
| Z-1891 | —CH₂CF₃ | —C(═O)NHCH₂C≡CCH₂CH₃ | Z-1892 | —Pr | —C(═O)NHCH₂C≡CCH₂CH₃ | 2-1893 | —c-Pr | —C(═O)NHCH₂C≡CCH₂CH₃ |
| Z-1894 | —CH₂CF₃ | —C(═O)NHC(CH₃)₂C≡CH | Z-1895 | —Pr | —C(═O)NHC(CH₃)₂C≡CH | Z-1896 | —c-Pr | —C(═O)NHC(CH₃)₂C≡CH |
| Z-1897 | —CH₂CF₃ | —C(═O)NHCH₂C≡CHCF₂H | Z-1898 | —Pr | —C(═O)NHCH₂C≡CHCF₂H | Z-1899 | —c-Pr | —C(═O)NHCH₂C≡CHCF₂H |
| Z-1900 | —CH₂CF₃ | —C(═O)NHCH₂C≡CCF₃ | Z-1901 | —Pr | —C(═O)NHCH₂C≡CCF₃ | Z-1902 | —c-Pr | —C(═O)NHCH₂C≡CCF₃ |
| Z-1903 | —CH₂CF₃ | —C(═O)H | Z-1904 | —Pr | —C(═O)H | Z-1905 | —c-Pr | —C(═O)H |
| Z-1906 | —CH₂CF₃ | —C(═O)Me | Z-1907 | —Pr | —C(═O)Me | Z-1908 | —c-Pr | —C(═O)Me |
| Z-1909 | —CH₂CF₃ | —C(═O)Et | Z-1910 | —Pr | —C(═O)Et | Z-1911 | —c-Pr | —C(═O)Et |
| Z-1912 | —CH₂CF₃ | —C(═O)Pr | Z-1913 | —Pr | —C(═O)Pr | Z-1914 | —c-Pr | —C(═O)Pr |
| Z-1915 | —CH₂CF₃ | —C(═O)—i-Pr | Z-1916 | —Pr | —C(═O)—i-Pr | Z-1917 | —c-Pr | —C(═O)—i-Pr |
| Z-1918 | —CH₂CF₃ | —C(═O)Bu | Z-1919 | —Pr | —C(═O)Bu | Z-1920 | —c-Pr | —C(═O)Bu |
| Z-1921 | —CH₂CF₃ | —C(═O)—sec-Bu | Z-1922 | —Pr | —C(═O)—sec-Bu | Z-1923 | —c-Pr | —C(═O)—sec-Bu |
| Z-1924 | —CH₂CF₃ | —C(═O)—i-Bu | Z-1925 | —Pr | —C(═O)—i-Bu | Z-1926 | —c-Pr | —C(═O)—i-Bu |
| Z-1927 | —CH₂CF₃ | —C(═O)—t-Bu | Z-1928 | —Pr | —C(═O)—t-Bu | Z-1929 | —c-Pr | —C(═O)—t-Bu |
| Z-1930 | —CH₂CF₃ | —C(═O)Pent | Z-1931 | —Pr | —C(═O)Pent | Z-1932 | —c-Pr | —C(═O)Pent |
| Z-1933 | —CH₂CF₃ | —C(═O)Hex | Z-1934 | —Pr | —C(═O)Hex | Z-1935 | —c-Pr | —C(═O)Hex |
| Z-1936 | —CH₂CF₃ | —C(═O)CH(CH₃)CH₂CH₂CH₃ | Z-1937 | —Pr | —C(═O)CH(CH₃)CH₂CH₂CH₃ | Z-1938 | —c-Pr | —C(═O)CH(CH₃)CH₂CH₂CH₃ |
| Z-1939 | —CH₂CF₃ | —C(═O)CH(CH₃)CH(CH₃)₂ | Z-1940 | —Pr | —C(═O)CH(CH₃)CH(CH₃)₂ | Z-1941 | —c-Pr | —C(═O)CH(CH₃)CH(CH₃)₂ |
| Z-1942 | —CH₂CF₃ | —C(═O)C(CH₃)₂CH₂CH₃ | Z-1943 | —Pr | —C(═O)C(CH₃)₂CH₂CH₃ | Z-1944 | —c-Pr | —C(═O)C(CH₃)₂CH₂CH₃ |
| Z-1945 | —CH₂CF₃ | —C(═O)CH(CH₂CH₃)₂ | Z-1946 | —Pr | —C(═O)CH(CH₂CH₃)₂ | Z-1947 | —c-Pr | —C(═O)CH(CH₂CH₃)₂ |
| Z-1948 | —CH₂CF₃ | —C(═O)CH₂CH₂CH(CH₃)₂ | Z-1949 | —Pr | —C(═O)CH₂CH₂CH(CH₃)₂ | Z-1950 | —c-Pr | —C(═O)CH₂CH₂CH(CH₃)₂ |
| Z-1951 | —CH₂CF₃ | —C(═O)CH₂CH₂CH₂CH(CH₃)₂ | Z-1952 | —Pr | —C(═O)CH₂CH₂CH₂CH(CH₃)₂ | 2-1953 | —c-Pr | —C(═O)CH₂CH₂CH₂CH(CH₃)₂ |
| Z-1954 | —CH₂CF₃ | —C(═O)CH₂CH₂CH(CH₃)CH₂CH₃ | Z-1955 | —Pr | —C(═O)CH₂CH₂CH(CH₃)CH₂CH₃ | Z-1956 | —c-Pr | —C(═O)CH₂CH₂CH(CH₃)CH₂CH₃ |
| Z-1957 | —CH₂CF₃ | —C(═O)CH₂CH(CH₃)CH₂CH₂CH₃ | Z-1958 | —Pr | —C(═O)CH₂CH(CH₃)CH₂CH₂CH₃ | Z-1959 | —c-Pr | —C(═O)CH₂CH(CH₃)CH₂CH₂CH₃ |
| Z-1960 | —CH₂CF₃ | —C(═O)CH(CH₃)CH₂CH₂CH₂CH₃ | Z-1961 | —Pr | —C(═O)CH(CH₃)CH₂CH₂CH₂CH₃ | Z-1962 | —c-Pr | —C(═O)CH(CH₃)CH₂CH₂CH₂CH₃ |
| Z-1963 | —CH₂CF₃ | —C(═O)CH(CH₃)CH₂CH(CH₃)₂ | Z-1964 | —Pr | —C(═O)CH(CH₃)CH₂CH(CH₃)₂ | Z-1965 | —c-Pr | —C(═O)CH(CH₃)CH₂CH(CH₃)₂ |
| Z-1966 | —CH₂CF₃ | —C(═O)CH(CH₃)CH(CH₃)CH₂CH₃ | Z-1967 | —Pr | —C(═O)CH(CH₃)CH(CH₃)CH₂CH₃ | Z-1968 | —c-Pr | —C(═O)CH(CH₃)CH(CH₃)CH₂CH₃ |
| Z-1969 | —CH₂CF₃ | —C(═O)C(CH₃)₂CH₂CH₂CH₃ | Z-1970 | —Pr | —C(═O)C(CH₃)₂CH₂CH₂CH₃ | Z-1971 | —c-Pr | —C(═O)C(CH₃)₂CH₂CH₂CH₃ |
| Z-1972 | —CH₂CF₃ | —C(═O)CH(CH₃)C(CH₃)₃ | Z-1973 | —Pr | —C(═O)CH(CH₃)C(CH₃)₃ | Z-1974 | —c-Pr | —C(═O)CH(CH₃)C(CH₃)₃ |
| Z-1975 | —CH₂CF₃ | —C(═O)C(CH₃)₂CH(CH₃)₂ | Z-1976 | —Pr | —C(═O)C(CH₃)₂CH(CH₃)₂ | Z-1977 | —c-Pr | —C(═O)C(CH₃)₂CH(CH₃)₂ |
| Z-1978 | —CH₂CF₃ | —C(═O)CH₂CH₂C(CH₃) | Z-1979 | —Pr | —C(═O)CH₂CH₂C(CH₃) | Z-1980 | —c-Pr | —C(═O)CH₂CH₂C(CH₃) |
| Z-1981 | —CH₂CF₃ | —C(═O)CH₂CH(CH₃)CH(CH₃)₂ | Z-1982 | —Pr | —C(═O)CH₂CH(CH₃)CH(CH₃)₂ | Z-1983 | —c-Pr | —C(═O)CH₂CH(CH₃)CH(CH₃)₂ |
| Z-1984 | —CH₂CF₃ | —C(═O)CH₂C(CH₃)₂CH₂CH₃ | Z-1985 | —Pr | —C(═O)CH₂C(CH₃)₂CH₂CH₃ | Z-1986 | —c-Pr | —C(═O)CH₂C(CH₃)₂CH₂CH₃ |
| Z-1987 | —CH₂CF₃ | —C(═O)CFH₂ | Z-1988 | —Pr | —C(═O)CFH₂ | Z-1989 | —c-Pr | —C(═O)CFH₂ |
| Z-1990 | —CH₂CF₃ | —C(═O)CF₂H | Z-1991 | —Pr | —C(═O)CF₂H | Z-1992 | —c-Pr | —C(═O)CF₂H |
| Z-1993 | —CH₂CF₃ | —C(═O)CF₂Cl | Z-1994 | —Pr | —C(═O)CF₂Cl | Z-1995 | —c-Pr | —C(═O)CF₂Cl |
| Z-1996 | —CH₂CF₃ | —C(═O)CF₃ | Z-1997 | —Pr | —C(═O)CF₃ | Z-1998 | —c-Pr | —C(═O)CF₃ |
| Z-1999 | —CH₂CF₃ | —C(═O)CH₂Cl | Z-2000 | —Pr | —C(═O)CH₂Cl | Z-2001 | —c-Pr | —C(═O)CH₂Cl |
| Z-2002 | —CH₂CF₃ | —C(═O)CHCl₂ | Z-2003 | —Pr | —C(═O)CHCl₂ | Z-2004 | —c-Pr | —C(═O)CHCl₂ |
| Z-2005 | —CH₂CF₃ | —C(═O)CCl₂F | Z-2006 | —Pr | —C(═O)CCl₂F | Z-2007 | —c-Pr | —C(═O)CCl₂F |
| Z-2008 | —CH₂CF₃ | —C(═O)CCl₃ | Z-2009 | —Pr | —C(═O)CCl₃ | Z-2010 | —c-Pr | —C(═O)CCl₃ |
| Z-2011 | —CH₂CF₃ | —C(═O)CH₂Br | Z-2012 | —Pr | —C(═O)CH₂Br | Z-2013 | —c-Pr | —C(═O)CH₂Br |
| Z-2014 | —CH₂CF₃ | —C(═O)CHBr₂ | Z-2015 | —Pr | —C(═O)CHBr₂ | Z-2016 | —c-Pr | —C(═O)CHBr₂ |
| Z-2017 | —CH₂CF₃ | —C(═O)CBr₃ | Z-2018 | —Pr | —C(═O)CBr₃ | Z-2019 | —c-Pr | —C(═O)CBr₃ |
| Z-2020 | —CH₂CF₃ | —C(═O)CH₂l | Z-2021 | —Pr | —C(═O)CH₂l | Z-2022 | —c-Pr | —C(═O)CH₂l |
| Z-2023 | —CH₂CF₃ | —C(═O)CHl₂ | Z-2024 | —Pr | —C(═O)CHl₂ | Z-2025 | —c-Pr | —C(═O)CHl₂ |
| Z-2026 | —CH₂CF₃ | —C(═O)CH₂CF₂H | Z-2027 | —Pr | —C(═O)CH₂CF₂H | Z-2028 | —c-Pr | —C(═O)CH₂CF₂H |
| Z-2029 | —CH₂CF₃ | —C(═O)CH₂CF₃ | Z-2030 | —Pr | —C(═O)CH₂CF₃ | Z-2031 | —c-Pr | —C(═O)CH₂CF₃ |
| Z-2032 | —CH₂CF₃ | —C(═O)CH₂CH₂CF₂H | Z-2033 | —Pr | —C(═O)CH₂CH₂CF₂H | Z-2034 | —c-Pr | —C(═O)CH₂CH₂CF₂H |
| Z-2035 | —CH₂CF₃ | —C(═O)CH₂CH₂CF₃ | Z-2036 | —Pr | —C(═O)CH₂CH₂CF₃ | Z-2037 | —c-Pr | —C(═O)CH₂CH₂CF₃ |
| Z-2038 | —CH₂CF₃ | —C(═O)CH₂CH₂CH₂CF₂H | Z-2039 | —Pr | —C(═O)CH₂CH₂CH₂CF₂H | Z-2040 | —c-Pr | —C(═O)CH₂CH₂CH₂CF₂H |
| Z-2041 | —CH₂CF₃ | —C(═O)CH₂CH₂CH₂CF₃ | Z-2042 | —Pr | —C(═O)CH₂CH₂CH₂CF₃ | Z-2043 | —c-Pr | —C(═O)CH₂CH₂CH₂CF₃ |
| Z-2044 | —CH₂CF₃ | —C(═O)CF₂CH₃ | Z-2045 | —Pr | —C(═O)CF₂CH₃ | Z-2046 | —c-Pr | —C(═O)CF₂CH₃ |
| Z-2047 | —CH₂CF₃ | —C(═O)CF₂CF₂H | Z-2048 | —Pr | —C(═O)CF₂CF₂H | Z-2049 | —c-Pr | —C(═O)CF₂CF₂H |
| Z-2050 | —CH₂CF₃ | —C(═O)CF₂CF₃ | Z-2051 | —Pr | —C(═O)CF₂CF₃ | Z-2052 | —c-Pr | —C(═O)CF₂CF₃ |
| Z-2053 | —CH₂CF₃ | —C(═O)CF₂CClF₂ | Z-2054 | —Pr | —C(═O)CF₂CClF₂ | Z-2055 | —c-Pr | —C(═O)CF₂CClF₂ |
| Z-2056 | —CH₂CF₃ | —C(═O)CFHCF₃ | Z-2057 | —Pr | —C(═O)CFHCF₃ | Z-2058 | —c-Pr | —C(═O)CFHCF₃ |
| Z-2059 | —CH₂CF₃ | —C(═O)CH₂CF₂CF₂H | Z-2060 | —Pr | —C(═O)CH₂CF₂CF₂H | Z-2061 | —c-Pr | —C(═O)CH₂CF₂CF₂H |
| Z-2062 | —CH₂CF₃ | —C(═O)CH₂CF₂CF₃ | Z-2063 | —Pr | —C(═O)CH₂CF₂CF₃ | Z-2064 | —c-Pr | —C(═O)CH₂CF₂CF₃ |
| Z-2065 | —CH₂CF₃ | —C(═O)CF₂CF₂CF₃ | Z-2066 | —Pr | —C(═O)CF₂CF₂CF₃ | Z-2067 | —c-Pr | —C(═O)CF₂CF₂CF₃ |
| Z-2068 | —CH₂CF₃ | —C(═O)CH₂CF₂CF₂CF₃ | Z-2069 | —Pr | —C(═O)CH₂CF₂CF₂CF₃ | Z-2070 | —c-Pr | —C(═O)CH₂CF₂CF₂CF₃ |
| Z-2071 | —CH₂CF₃ | —C(═O)CF₂CF₂CF₂CF₃ | Z-2072 | —Pr | —C(═O)CF₂CF₂CF₂CF₃ | Z-2073 | —c-Pr | —C(═O)CF₂CF₂CF₂CF₃ |
| Z-2074 | —CH₂CF₃ | —C(═O)CH₂CF₂CF₂CF₂CF₃ | Z-2075 | —Pr | —C(═O)CH₂CF₂CF₂CF₂CF₃ | Z-2076 | —c-Pr | —C(═O)CH₂CF₂CF₂CF₂CF₃ |
| Z-2077 | —CH₂CF₃ | —C(═O)CF₂CF₂CF₂CF₂CF₃ | Z-2078 | —Pr | —C(═O)CF₂CF₂CF₂CF₂CF₃ | Z-2079 | —c-Pr | —C(═O)CF₂CF₂CF₂CF₂CF₃ |
| Z-2080 | —CH₂CF₃ | —C(═O)—c-Pr | Z-2081 | —Pr | —C(═O)—c-Pr | Z-2082 | —c-Pr | —C(═O)—c-Pr |
| Z-2083 | —CH₂CF₃ | —C(═O)—c-Bu | Z-2084 | —Pr | —C(═O)—c-Bu | Z-2085 | —c-Pr | —C(═O)—c-Bu |
| Z-2086 | —CH₂CF₃ | —C(═O)—c-Pent | Z-2087 | —Pr | —C(═O)—c-Pent | Z-2088 | —c-Pr | —C(═O)—c-Pent |
| Z-2089 | —CH₂CF₃ | —C(═O)—c-Hex | Z-2090 | —Pr | —C(═O)—c-Hex | Z-2091 | —c-Pr | —C(═O)—c-Hex |
| Z-2092 | —CH₂CF₃ | —C(═O)—c-Hept | Z-2093 | —Pr | —C(═O)—c-Hept | Z-2094 | —c-Pr | —C(═O)—c-Hept |
| Z-2095 | —CH₂CF₃ | —C(═O)—c-Oct | Z-2096 | —Pr | —C(═O)—c-Oct | Z-2097 | —c-Pr | —C(═O)—c-Oct |
| Z-2098 | —CH₂CF₃ | —C(═O)CH═CH₂ | Z-2099 | —Pr | —C(═O)CH═CH₂ | Z-2100 | —c-Pr | —C(═O)CH═CH₂ |
| Z-2101 | —CH₂CF₃ | —C(═O)CH₂CH═CH₂ | Z-2102 | —Pr | —C(═O)CH₂CH═CH₂ | Z-2103 | —c-Pr | —C(═O)CH₂CH═CH₂ |
| Z-2104 | —CH₂CF₃ | —C(═O)CH═CHCH₃ | Z-2105 | —Pr | —C(═O)CH═CHCH₃ | Z-2106 | —c-Pr | —C(═O)CH═CHCH₃ |
| Z-2107 | —CH₂CF₃ | —C(═O)CH₂C(CH₃)═CH₂ | Z-2108 | —Pr | —C(═O)CH₂C(CH₃)═CH₂ | Z-2109 | —c-Pr | —C(═O)CH₂C(CH₃)═CH₂ |
| Z-2110 | —CH₂CF₃ | —C(═O)CH₂CH₂CH═CH₂ | Z-2111 | —Pr | —C(═O)CH₂CH₂CH═CH₂ | Z-2112 | —c-Pr | —C(═O)CH₂CH₂CH═CH₂ |
| Z-2113 | —CH₂CF₃ | —C(═O)CH₂CH═CHCH₃ | Z-2114 | —Pr | —C(═O)CH₂CH═CHCH₃ | Z-2115 | —c-Pr | —C(═O)CH₂CH═CHCH₃ |
| Z-2116 | —CH₂CF₃ | —C(═O)CH═CHCH₂CH₃ | Z-2117 | —Pr | —C(═O)CH═CHCH₂CH₃ | Z-2118 | —c-Pr | —C(═O)CH═CHCH₂CH₃ |
| Z-2119 | —CH₂CF₃ | —C(═O)CH₂CH═C(CH₃)₂ | Z-2120 | —Pr | —C(═O)CH₂CH═C(CH₃)₂ | Z-2121 | —c-Pr | —C(═O)CH₂CH═C(CH₃)₂ |
| Z-2122 | —CH₂CF₃ | —C(═O)CH₂CH₂CH═C(CH₃)₂ | Z-2123 | —Pr | —C(═O)CH₂CH₂CH═C(CH₃)₂ | Z-2124 | —c-Pr | —C(═O)CH₂CH₂CH═C(CH₃)₂ |
| Z-2125 | —CH₂CF₃ | —C(═O)CH═CFH | Z-2126 | —Pr | —C(═O)CH═CFH | Z-2127 | —c-Pr | —C(═O)CH═CFH |
| Z-2128 | —CH₂CF₃ | —C(═O)CH═CF₂ | Z-2129 | —Pr | —C(═O)CH═CF₂ | Z-2130 | —c-Pr | —C(═O)CH═CF₂ |
| Z-2131 | —CH₂CF₃ | —C(═O)CH═CCl₂ | Z-2132 | —Pr | —C(═O)CH═CCl₂ | Z-2133 | —c-Pr | —C(═O)CH═CCl₂ |
| Z-2134 | —CH₂CF₃ | —C(═O)CH₂CH═CFH | Z-2135 | —Pr | —C(═O)CH₂CH═CFH | Z-2136 | —c-Pr | —C(═O)CH₂CH═CFH |
| Z-2137 | —CH₂CF₃ | —C(═O)CH₂CH═CF₂ | Z-2138 | —Pr | —C(═O)CH₂CH═CF₂ | Z-2139 | —c-Pr | —C(═O)CH₂CH═CF₂ |
| Z-2140 | —CH₂CF₃ | —C(═O)CH₂CH═CCl₂ | Z-2141 | —Pr | —C(═O)CH₂CH═CCl₂ | 2-2142 | —c-Pr | —C(═O)CH₂CH═CCl₂ |
| Z-2143 | —CH₂CF₃ | —C(═O)CH₂CH₂CH═CF₂ | Z-2144 | —Pr | —C(═O)CH₂CH₂CH═CF₂ | Z-2145 | —c-Pr | —C(═O)CH₂CH₂CH═CF₂ |
| Z-2146 | —CH₂CF₃ | —C(═O)CH₂CH₂CH₂CH═CF₂ | Z-2147 | —Pr | —C(═O)CH₂CH₂CH₂CH═CF₂ | Z-2148 | —c-Pr | —C(═O)CH₂CH₂CH₂CH═CF₂ |
| Z-2149 | —CH₂CF₃ | —C(═O)CH₂CH₂CH₂CH₂CH═CF₂ | Z-2150 | —Pr | —C(═O)CH₂CH₂CH₂CH₂CH═CF₂ | Z-2151 | —c-Pr | —C(═O)CH₂CH₂CH₂CH₂CH═CF₂ |
| Z-2152 | —CH₂CF₃ | —C(═O)C≡CH | Z-2153 | —Pr | —C(═O)C≡CH | Z-2154 | —c-Pr | —C(═O)C≡CH |
| Z-2155 | —CH₂CF₃ | —C(═O)C≡CCH₃ | Z-2156 | —Pr | —C(═O)C≡CCH₃ | Z-2157 | —c-Pr | —C(═O)C≡CCH₃ |
| Z-2158 | —CH₂CF₃ | —C(═O)CH₂C≡CH | Z-2159 | —Pr | —C(═O)CH₂C≡CH | Z-2160 | —c-Pr | —C(═O)CH₂C≡CH |
| Z-2161 | —CH₂CF₃ | —C(═O)C≡CCH₂CH₃ | Z-2162 | —Pr | —C(═O)C≡CCH₂CH₃ | Z-2163 | —c-Pr | —C(═O)C≡CCH₂CH₃ |
| Z-2164 | —CH₂CF₃ | —C(═O)CH₂C≡CCH₃ | Z-2165 | —Pr | —C(═O)CH₂C≡CCH₃ | Z-2166 | —c-Pr | —C(═O)CH₂C≡CCH₃ |
| Z-2167 | —CH₂CF₃ | —C(═O)CH₂CH₂C≡CH | Z-2168 | —Pr | —C(═O)CH₂CH₂C≡CH | Z-2169 | —c-Pr | —C(═O)CH₂CH₂C≡CH |
| Z-2170 | —CH₂CF₃ | —C(═O)C≡CCH₂CH₂CH₃ | Z-2171 | —Pr | —C(═O)C≡CCH₂CH₂CH₃ | Z-2172 | —c-Pr | —C(═O)C≡CCH₂CH₂CH₃ |
| Z-2173 | —CH₂CF₃ | —C(═O)CH₂C≡CCH₂CH₃ | Z-2174 | —Pr | —C(═O)CH₂C≡CCH₂CH₃ | Z-2175 | —c-Pr | —C(═O)CH₂C≡CCH₂CH₃ |
| Z-2176 | —CH₂CF₃ | —C(═O)C(CH₃)₂C≡CH | Z-2177 | —Pr | —C(═O)C(CH₃)₂C≡CH | Z-2178 | —c-Pr | —C(═O)C(CH₃)₂C≡CH |
| Z-2179 | —CH₂CF₃ | —C(═O)C≡CF | Z-2180 | —Pr | —C(═O)C≡CF | Z-2181 | —c-Pr | —C(═O)C≡CF |
| Z-2182 | —CH₂CF₃ | —C(═O)C≡CCF₂H | Z-2183 | —Pr | —C(═O)C≡CCF₂H | Z-2184 | —c-Pr | —C(═O)C≡CCF₂H |
| Z-2185 | —CH₂CF₃ | —C(═O)C≡CCF₃ | Z-2186 | —Pr | —C(═O)C≡CCF₃ | Z-2187 | —c-Pr | —C(═O)C≡CCF₃ |
| Z-2188 | —CH₂CF₃ | —C(═O)C≡CCH₂CF₂H | Z-2189 | —Pr | —C(═O)C≡CCH₂CF₂H | Z-2190 | —c-Pr | —C(═O)C≡CCH₂CF₂H |
| Z-2191 | —CH₂CF₃ | —C(═O)C≡CCH₂CF₃ | Z-2192 | —Pr | —C(═O)C≡CCH₂CF₃ | Z-2193 | —c-Pr | —C(═O)C≡CCH₂CF₃ |
| Z-2194 | —CH₂CF₃ | —C(═O)CH₂C≡CHCF₂H | Z-2195 | —Pr | —C(═O)CH₂C≡CHCF₂H | Z-2196 | —c-Pr | —C(═O)CH₂C≡CHCF₂H |
| Z-2197 | —CH₂CF₃ | —C(═O)CH₂C≡CCF₃ | Z-2198 | —Pr | —C(═O)CH₂C≡CCF₃ | Z-2199 | —c-Pr | —C(═O)CH₂C≡CCF₃ |
| Z-2200 | —CH₂CF₃ | —C(═O)CH₂C≡N | Z-2201 | —Pr | —C(═O)CH₂C≡N | Z-2202 | —c-Pr | —C(═O)CH₂C≡N |
| Z-2203 | —CH₂CF₃ | —C(═O)C(Me)C≡N | Z-2204 | —Pr | —C(═O)C(Me)C≡N | Z-2205 | —c-Pr | —C(═O)C(Me)C≡N |
| Z-2206 | —CH₂CF₃ | —C(═O)CH₂CH₂C≡N | Z-2207 | —Pr | —C(═O)CH₂CH₂C≡N | Z-2208 | —c-Pr | —C(═O)CH₂CH₂C≡N |
| Z-2209 | —CH₂CF₃ | —C(═O)CH₂CH₂CH₂C≡N | Z-2210 | —Pr | —C(═O)CH₂CH₂CH₂C≡N | Z-2211 | —c-Pr | —C(═O)CH₂CH₂CH₂C≡N |
| Z-2212 | —CH₂CF₃ | —C(═O)CH₂OH | Z-2213 | —Pr | —C(═O)CH₂OH | Z-2214 | —c-Pr | —C(═O)CH₂OH |
| Z-2215 | —CH₂CF₃ | —C(═O)CH₂OMe | Z-2216 | —Pr | —C(═O)CH₂OMe | Z-2217 | —c-Pr | —C(═O)CH₂OMe |
| Z-2218 | —CH₂CF₃ | —C(═O)CH₂OEt | Z-2219 | —Pr | —C(═O)CH₂OEt | Z-2220 | —c-Pr | —C(═O)CH₂OEt |
| Z-2221 | —CH₂CF₃ | —C(═O)CH₂OPr | Z-2222 | —Pr | —C(═O)CH₂OPr | Z-2223 | —c-Pr | —C(═O)CH₂OPr |
| Z-2224 | —CH₂CF₃ | —C(═O)CH₂CH₂OMe | Z-2225 | —Pr | —C(═O)CH₂CH₂OMe | Z-2226 | —c-Pr | —C(═O)CH₂CH₂OMe |
| Z-2227 | —CH₂CF₃ | —C(═O)CH₂CH₂OEt | Z-2228 | —Pr | —C(═O)CH₂CH₂OEt | Z-2229 | —c-Pr | —C(═O)CH₂CH₂OEt |
| Z-2230 | —CH₂CF₃ | —C(═O)CH₂—(1-Pyra) | Z-2231 | —Pr | —C(═O)CH₂—(1-Pyra) | Z-2232 | —c-Pr | —C(═O)CH₂—(1-Pyra) |
| Z-2233 | —CH₂CF₃ | —C(═O)CH₂—(1-Tria) | Z-2234 | —Pr | —C(═O)CH₂—(1-Tria) | Z-2235 | —c-Pr | —C(═O)CH₂—(1-Tria) |
| Z-2236 | —CH₂CF₃ | —C(═O)OH | Z-2237 | —Pr | —C(═O)OH | Z-2238 | —c-Pr | —C(═O)OH |
| Z-2239 | —CH₂CF₃ | —C(═O)OMe | Z-2240 | —Pr | —C(═O)OMe | Z-2241 | —c-Pr | —C(═O)OMe |
| Z-2242 | —CH₂CF₃ | —C(═O)OEt | Z-2243 | —Pr | —C(═O)OEt | Z-2244 | —c-Pr | —C(═O)OEt |
| Z-2245 | —CH₂CF₃ | —C(═O)OPr | Z-2246 | —Pr | —C(═O)OPr | Z-2247 | —c-Pr | —C(═O)OPr |
| Z-2248 | —CH₂CF₃ | —C(═O)O—i-Pr | Z-2249 | —Pr | —C(═O)O—i-Pr | Z-2250 | —c-Pr | —C(═O)O—i-Pr |
| Z-2251 | —CH₂CF₃ | —C(═O)OBu | Z-2252 | —Pr | —C(═O)OBu | Z-2253 | —c-Pr | —C(═O)OBu |
| Z-2254 | —CH₂CF₃ | —C(═O)O—sec-Bu | Z-2255 | —Pr | —C(═O)O—sec-Bu | Z-2256 | —c-Pr | —C(═O)O—sec-Bu |
| Z-2257 | —CH₂CF₃ | —C(═O)O—i-Bu | Z-2258 | —Pr | —C(═O)O—i-Bu | Z-2259 | —c-Pr | —C(═O)O—i-Bu |
| Z-2260 | —CH₂CF₃ | —C(═O)O—t-Bu | Z-2261 | —Pr | —C(═O)O—t-Bu | Z-2262 | —c-Pr | —C(═O)O—t-Bu |
| Z-2263 | —CH₂CF₃ | —C(═O)OPent | Z-2264 | —Pr | —C(═O)OPent | Z-2265 | —c-Pr | —C(═O)OPent |
| Z-2266 | —CH₂CF₃ | —C(═O)OHex | Z-2267 | —Pr | —C(═O)OHex | Z-2268 | —c-Pr | —C(═O)OHex |
| Z-2269 | —CH₂CF₃ | —C(═O)OCH(CH₃)CH₂CH₂CH₃ | Z-2270 | —Pr | —C(═O)OCH(CH₃)CH₂CH₂CH₃ | Z-2271 | —c-Pr | —C(═O)OCH(CH₃)CH₂CH₂CH₃ |
| Z-2272 | —CH₂CF₃ | —C(═O)OCH(CH₃)CH(CH₃)₂ | Z-2273 | —Pr | —C(═O)OCH(CH₃)CH(CH₃)₂ | Z-2274 | —c-Pr | —C(═O)OCH(CH₃)CH(CH₃)₂ |
| Z-2275 | —CH₂CF₃ | —C(═O)OC(CH₃)₂CH₂CH₃ | Z-2276 | —Pr | —C(═O)OC(CH₃)₂CH₂CH₃ | Z-2277 | —c-Pr | —C(═O)OC(CH₃)₂CH₂CH₃ |
| Z-2278 | —CH₂CF₃ | —C(═O)OCH(CH₂CH₃)₂ | Z-2279 | —Pr | —C(═O)OCH(CH₂CH₃)₂ | Z-2280 | —c-Pr | —C(═O)OCH(CH₂CH₃)₂ |
| Z-2281 | —CH₂CF₃ | —C(═O)OCH₂CH₂CH(CH₃)₂ | Z-2282 | —Pr | —C(═O)OCH₂CH₂CH(CH₃)₂ | Z-2283 | —c-Pr | —C(═O)OCH₂CH₂CH(CH₃)₂ |
| Z-2284 | —CH₂CF₃ | —C(═O)OCH₂CH₂CH₂CH(CH₃)₂ | Z-2285 | —Pr | —C(═O)OCH₂CH₂CH₂CH(CH₃)₂ | Z-2286 | —c-Pr | —C(═O)OCH₂CH₂CH₂CH(CH₃)₂ |
| Z-2287 | —CH₂CF₃ | —C(═O)OCH₂CH₂CH(CH₃)CH₂CH₃ | Z-2288 | —Pr | —C(═O)OCH₂CH₂CH(CH₃)CH₂CH₃ | Z-2289 | —c-Pr | —C(═O)OCH₂CH₂CH(CH₃)CH₂CH₃ |
| Z-2290 | —CH₂CF₃ | —C(═O)OCH₂CH(CH₃)CH₂CH₂CH₃ | Z-2291 | —Pr | —C(═O)OCH₂CH(CH₃)CH₂CH₂CH₃ | Z-2292 | —c-Pr | —C(═O)OCH₂CH(CH₃)CH₂CH₂CH₃ |
| Z-2293 | —CH₂CF₃ | —C(═O)OCH(CH₃)CH₂CH₂CH₂CH₃ | Z-2294 | —Pr | —C(═O)OCH(CH₃)CH₂CH₂CH₂CH₃ | Z-2295 | —c-Pr | —C(═O)OCH(CH₃)CH₂CH₂CH₂CH₃ |
| Z-2296 | —CH₂CF₃ | —C(═O)OCH(CH₃)CH₂CH(CH₃)₂ | Z-2297 | —Pr | —C(═O)OCH(CH₃)CH₂CH(CH₃)₂ | Z-2298 | —c-Pr | —C(═O)OCH(CH₃)CH₂CH(CH₃)₂ |
| Z-2299 | —CH₂CF₃ | —C(═O)OCH(CH₃)CH(CH₃)CH₂CH₃ | Z-2300 | —Pr | —C(═O)OCH(CH₃)CH(CH₃)CH₂CH₃ | Z-2301 | —c-Pr | —C(═O)OCH(CH₃)CH(CH₃)CH₂CH₃ |
| Z-2302 | —CH₂CF₃ | —C(═O)OC(CH₃)₂CH₂CH₂CH₃ | Z-2303 | —Pr | —C(═O)OC(CH₃)₂CH₂CH₂CH₃ | Z-2304 | —c-Pr | —C(═O)OC(CH₃)₂CH₂CH₂CH₃ |
| Z-2305 | —CH₂CF₃ | —C(═O)OCH(CH₃)C(CH₃)₃ | Z-2306 | —Pr | —C(═O)OCH(CH₃)C(CH₃)₃ | Z-2307 | —c-Pr | —C(═O)OCH(CH₃)C(CH₃)₃ |
| Z-2308 | —CH₂CF₃ | —C(═O)OC(CH₃)₂CH(CH₃)₂ | Z-2309 | —Pr | —C(═O)OC(CH₃)₂CH(CH₃)₂ | Z-2310 | —c-Pr | —C(═O)OC(CH₃)₂CH(CH₃)₂ |
| Z-2311 | —CH₂CF₃ | —C(═O)OCH₂CH₂C(CH₃)₃ | Z-2312 | —Pr | —C(═O)OCH₂CH₂C(CH₃)₃ | Z-2313 | —c-Pr | —C(═O)OCH₂CH₂C(CH₃)₃ |
| Z-2314 | —CH₂CF₃ | —C(═O)OCH₂CH(CH₃)CH(CH₃)₂ | Z-2315 | —Pr | —C(═O)OCH₂CH(CH₃)CH(CH₃)₂ | Z-2316 | —c-Pr | —C(═O)OCH₂CH(CH₃)CH(CH₃)₂ |
| Z-2317 | —CH₂CF₃ | —C(═O)OCH₂C(CH₃)₂CH₂CH₃ | Z-2318 | —Pr | —C(═O)OCH₂C(CH₃)₂CH₂CH₃ | Z-2319 | —c-Pr | —C(═O)OCH₂C(CH₃)₂CH₂CH₃ |
| Z-2320 | —CH₂CF₃ | —C(═O)OCFH₂ | Z-2321 | —Pr | —C(═O)OCFH₂ | Z-2322 | —c-Pr | —C(═O)OCFH₂ |
| Z-2323 | —CH₂CF₃ | —C(═O)OCF₂H | Z-2324 | —Pr | —C(═O)OCF₂H | Z-2325 | —c-Pr | —C(═O)OCF₂H |
| Z-2326 | —CH₂CF₃ | —C(═O)OCF₃ | Z-2327 | —Pr | —C(═O)OCF₃ | Z-2328 | —c-Pr | —C(═O)OCF₃ |
| Z-2329 | —CH₂CF₃ | —C(═O)OCH₂Cl | Z-2330 | —Pr | —C(═O)OCH₂Cl | Z-2331 | —c-Pr | —C(═O)OCH₂Cl |
| Z-2332 | —CH₂CF₃ | —C(═O)OCHCl₂ | Z-2333 | —Pr | —C(═O)OCHCl₂ | Z-2334 | —c-Pr | —C(═O)OCHCl₂ |
| Z-2335 | —CH₂CF₃ | —C(═O)OCCl₃ | Z-2336 | —Pr | —C(═O)OCCl₃ | Z-2337 | —c-Pr | —C(═O)OCCl₃ |
| Z-2338 | —CH₂CF₃ | —C(═O)OCH₂Br | Z-2339 | —Pr | —C(═O)OCH₂Br | Z-2340 | —c-Pr | —C(═O)OCH₂Br |
| Z-2341 | —CH₂CF₃ | —C(═O)OCHBr₂ | Z-2342 | —Pr | —C(═O)OCHBr₂ | Z-2343 | —c-Pr | —C(═O)OCHBr₂ |
| Z-2344 | —CH₂CF₃ | —C(═O)OCBr₃ | Z-2345 | —Pr | —C(═O)OCBr₃ | Z-2346 | —c-Pr | —C(═O)OCBr₃ |
| Z-2347 | —CH₂CF₃ | —C(═O)OCH₂l | Z-2348 | —Pr | —C(═O)OCH₂l | Z-2349 | —c-Pr | —C(═O)OCH₂l |
| Z-2350 | —CH₂CF₃ | —C(═O)OCHl₂ | Z-2351 | —Pr | —C(═O)OCHl₂ | Z-2352 | —c-Pr | —C(═O)OCHl₂ |
| Z-2353 | —CH₂CF₃ | —C(═O)OCH₂CF₂H | Z-2354 | —Pr | —C(═O)OCH₂CF₂H | Z-2355 | —c-Pr | —C(═O)OCH₂CF₂H |
| Z-2356 | —CH₂CF₃ | —C(═O)OCH₂CF₃ | Z-2357 | —Pr | —C(═O)OCH₂CF₃ | Z-2358 | —c-Pr | —C(═O)OCH₂CF₃ |
| Z-2359 | —CH₂CF₃ | —C(═O)OCH₂CH₂CF₂H | Z-2360 | —Pr | —C(═O)OCH₂CH₂CF₂H | Z-2361 | —c-Pr | —C(═O)OCH₂CH₂CF₂H |
| Z-2362 | —CH₂CF₃ | —C(═O)OCH₂CH₂CF₃ | Z-2363 | —Pr | —C(═O)OCH₂CH₂CF₃ | Z-2364 | —c-Pr | —C(═O)OCH₂CH₂CF₃ |
| Z-2365 | —CH₂CF₃ | —C(═O)OCH₂CH₂CH₂CF₂H | Z-2366 | —Pr | —C(═O)OCH₂CH₂CH₂CF₂H | Z-2367 | —c-Pr | —C(═O)OCH₂CH₂CH₂CF₂H |
| Z-2368 | —CH₂CF₃ | —C(═O)OCH₂CH₂CH₂CF₃ | Z-2369 | —Pr | —C(═O)OCH₂CH₂CH₂CF₃ | Z-2370 | —c-Pr | —C(═O)OCH₂CH₂CH₂CF₃ |
| Z-2371 | —CH₂CF₃ | —C(═O)OCF₂CF₂H | Z-2372 | —Pr | —C(═O)OCF₂CF₂H | Z-2373 | —c-Pr | —C(═O)OCF₂CF₂H |
| Z-2374 | —CH₂CF₃ | —C(═O)OCF₂CF₃ | Z-2375 | —Pr | —C(═O)OCF₂CF₃ | Z-2376 | —c-Pr | —C(═O)OCF₂CF₃ |
| Z-2377 | —CH₂CF₃ | —C(═O)OCFHCF₃ | Z-2378 | —Pr | —C(═O)OCFHCF₃ | Z-2379 | —c-Pr | —C(═O)OCFHCF₃ |
| Z-2380 | —CH₂CF₃ | —C(═O)OCH₂CF₂CF₂H | Z-2381 | —Pr | —C(═O)OCH₂CF₂CF₂H | Z-2382 | —c-Pr | —C(═O)OCH₂CF₂CF₂H |
| Z-2383 | —CH₂CF₃ | —C(═O)OCH₂CF₂CF₃ | Z-2384 | —Pr | —C(═O)OCH₂CF₂CF₃ | Z-2385 | —c-Pr | —C(═O)OCH₂CF₂CF₃ |
| Z-2386 | —CH₂CF₃ | —C(═O)OCF₂CF₂CF₃ | Z-2387 | —Pr | —C(═O)OCF₂CF₂CF₃ | Z-2388 | —c-Pr | —C(═O)OCF₂CF₂CF₃ |
| Z-2389 | —CH₂CF₃ | —C(═O)OCH₂CF₂CF₂CF₃ | Z-2390 | —Pr | —C(═O)OCH₂CF₂CF₂CF₃ | Z-2391 | —c-Pr | —C(═O)OCH₂CF₂CF₂CF₃ |
| Z-2392 | —CH₂CF₃ | —C(═O)OCF₂CF₂CF₂CF₃ | Z-2393 | —Pr | —C(═O)OCF₂CF₂CF₂CF₃ | Z-2394 | —c-Pr | —C(═O)OCF₂CF₂CF₂CF₃ |
| Z-2395 | —CH₂CF₃ | —C(═O)OCH₂CF₂CF₂CF₂CF₃ | Z-2396 | —Pr | —C(═O)OCH₂CF₂CF₂CF₂CF₃ | Z-2397 | —c-Pr | —C(═O)OCH₂CF₂CF₂CF₂CF₃ |
| Z-2398 | —CH₂CF₃ | —C(═O)O—c-Pr | Z-2399 | —Pr | —C(═O)O—c-Pr | Z-2400 | —c-Pr | —C(═O)O—c-Pr |
| Z-2401 | —CH₂CF₃ | —C(═O)O—c-Bu | Z-2402 | —Pr | —C(═O)O—c-Bu | Z-2403 | —c-Pr | —C(═O)O—c-Bu |
| Z-2404 | —CH₂CF₃ | —C(═O)O—c-Pent | Z-2405 | —Pr | —C(═O)O—c-Pent | Z-2406 | —c-Pr | —C(═O)O—c-Pent |
| Z-2407 | —CH₂CF₃ | —C(═O)O—c-Hex | Z-2408 | —Pr | —C(═O)O—c-Hex | Z-2409 | —c-Pr | —C(═O)O—c-Hex |
| Z-2410 | —CH₂CF₃ | —C(═O)O—c-Hept | Z-2411 | —Pr | —C(═O)O—c-Hept | Z-2412 | —c-Pr | —C(═O)O—c-Hept |
| Z-2413 | —CH₂CF₃ | —C(═O)O—c-Oct | Z-2414 | —Pr | —C(═O)O—c-Oct | Z-2415 | —c-Pr | —C(═O)O—c-Oct |
| 2-2416 | —CH₂CF₃ | —C(═O)OCH═CH₂ | Z-2417 | —Pr | —C(═O)OCH═CH₂ | Z-2418 | —c-Pr | —C(═O)OCH═CH₂ |
| Z-2419 | —CH₂CF₃ | —C(═O)OCH₂CH═CH₂ | Z-2420 | —Pr | —C(═O)OCH₂CH═CH₂ | Z-2421 | —c-Pr | —C(═O)OCH₂CH═CH₂ |
| Z-2422 | —CH₂CF₃ | —C(═O)OCH═CHCH₃ | Z-2423 | —Pr | —C(═O)OCH═CHCH₃ | Z-2424 | —c-Pr | —C(═O)OCH═CHCH₃ |
| Z-2425 | —CH₂CF₃ | —C(═O)OCH₂C(CH₃)═CH₂ | Z-2426 | —Pr | —C(═O)OCH₂C(CH₃)═CH₂ | Z-2427 | —c-Pr | —C(═O)OCH₂C(CH₃)═CH₂ |
| Z-2428 | —CH₂CF₃ | —C(═O)OCH₂CH₂CH═CH₂ | Z-2429 | —Pr | —C(═O)OCH₂CH₂CH═CH₂ | Z-2430 | —c-Pr | —C(═O)OCH₂CH₂CH═CH₂ |
| Z-2431 | —CH₂CF₃ | —C(═O)OCH₂CH═CHCH₃ | Z-2432 | —Pr | —C(═O)OCH₂CH═CHCH₃ | Z-2433 | —c-Pr | —C(═O)OCH₂CH═CHCH₃ |
| Z-2434 | —CH₂CF₃ | —C(═O)OCH═CHCH₂CH₃ | Z-2435 | —Pr | —C(═O)OCH═CHCH₂CH₃ | Z-2436 | —c-Pr | —C(═O)OCH═CHCH₂CH₃ |
| Z-2437 | —CH₂CF₃ | —C(═O)OCH₂CH═C(CH₃)₂ | Z-2438 | —Pr | —C(═O)OCH₂CH═C(CH₃)₂ | Z-2439 | —c-Pr | —C(═O)OCH₂CH═C(CH₃)₂ |
| Z-2440 | —CH₂CF₃ | —C(═O)OCH2CH₂CH═C(CH₃)₂ | Z-2441 | —Pr | —C(═O)OCH2CH₂CH═C(CH₃)₂ | Z-2442 | —c-Pr | —C(═O)OCH2CH₂CH═C(CH₃)₂ |
| Z-2443 | —CH₂CF₃ | —C(═O)OCH═CFH | Z-2444 | —Pr | —C(═O)OCH═CFH | Z-2445 | —c-Pr | —C(═O)OCH═CFH |
| Z-2446 | —CH₂CF₃ | —C(═O)OCH═CF₂ | Z-2447 | —Pr | —C(═O)OCH═CF₂ | Z-2448 | —c-Pr | —C(═O)OCH═CF₂ |
| Z-2449 | —CH₂CF₃ | —C(═O)OCH═CCl₂ | Z-2450 | —Pr | —C(═O)OCH═CCl₂ | Z-2451 | —c-Pr | —C(═O)OCH═CCl₂ |
| Z-2452 | —CH₂CF₃ | —C(═O)OCH₂CH═CFH | Z-2453 | —Pr | —C(═O)OCH₂CH═CFH | Z-2454 | —c-Pr | —C(═O)OCH₂CH═CFH |
| Z-2455 | —CH₂CF₃ | —C(═O)OCH₂CH═CF₂ | Z-2456 | —Pr | —C(═O)OCH₂CH═CF₂ | Z-2457 | —c-Pr | —C(═O)OCH₂CH═CF₂ |
| Z-2458 | —CH₂CF₃ | —C(═O)OCH₂CH═CCl₂ | Z-2459 | —Pr | —C(═O)OCH₂CH═CCl₂ | Z-2460 | —c-Pr | —C(═O)OCH₂CH═CCl₂ |
| Z-2461 | —CH₂CF₃ | —C(═O)OCH₂CH₂CH═CF₂ | Z-2462 | —Pr | —C(═O)OCH₂CH₂CH═CF₂ | Z-2463 | —c-Pr | —C(═O)OCH₂CH₂CH═CF₂ |
| Z-2464 | —CH₂CF₃ | —C(═O)OCH₂CH₂CH₂CH═CF₂ | Z-2465 | —Pr | —C(═O)OCH₂CH₂CH₂CH═CF₂ | Z-2466 | —c-Pr | —C(═O)OCH₂CH₂CH₂CH═CF₂ |
| Z-2467 | —CH₂CF₃ | —C(═O)OCH₂CH₂CH₂CH₂CH═CF₂ | Z-2468 | —Pr | —C(═O)OCH₂CH₂CH₂CH₂CH═CF₂ | Z-2469 | —c-Pr | —C(═O)OCH₂CH₂CH₂CH₂CH═CF₂ |
| Z-2470 | —CH₂CF₃ | —C(═Q)OCH₂C≡CH | Z-2471 | —Pr | —C(═Q)OCH₂C≡CH | Z-2472 | —c-Pr | —C(═Q)OCH₂C≡CH |
| Z-2473 | —CH₂CF₃ | —C(═O)OCH₂C≡CCH₃ | Z-2474 | —Pr | —C(═O)OCH₂C≡CCH₃ | Z-2475 | —c-Pr | —C(═O)OCH₂C≡CCH₃ |
| Z-2476 | —CH₂CF₃ | —C(═O)OCH₂CH₂C≡CH | Z-2477 | —Pr | —C(═O)OCH₂CH₂C≡CH | Z-2478 | —c-Pr | —C(═O)OCH₂CH₂C≡CH |
| Z-2479 | —CH₂CF₃ | —C(═O)OCH₂C≡CCH₂CH₃ | Z-2480 | —Pr | —C(═O)OCH₂C≡CCH₂CH₃ | Z-2481 | —c-Pr | —C(═O)OCH₂C≡CCH₂CH₃ |
| Z-2482 | —CH₂CF₃ | —C(═O)OC(CH₃)₂C≡CH | Z-2483 | —Pr | —C(═O)OC(CH₃)₂C≡CH | Z-2484 | —c-Pr | —C(═O)OC(CH₃)₂C≡CH |
| Z-2485 | —CH₂CF₃ | —C(═O)OCH₂C≡CHCF₂H | Z-2486 | —Pr | —C(═O)OCH₂C≡CHCF₂H | Z-2487 | —c-Pr | —C(═O)OCH₂C≡CHCF₂H |
| Z-2488 | —CH₂CF₃ | —C(═O)OCH₂C≡CCF₃ | Z-2489 | —Pr | —C(═O)OCH₂C≡CCF₃ | Z-2490 | —c-Pr | —C(═O)OCH₂C≡CCF₃ |
| Z-2491 | —CH₂CF₃ | —C(═O)Ph | Z-2492 | —Pr | —C(═O)Ph | Z-2493 | —c-Pr | —C(═O)Ph |
| Z-2494 | —CH₂CF₃ | —C(═O)(2-Py) | Z-2495 | —Pr | —C(═O)(2-Py) | Z-2496 | —c-Pr | —C(═O)(2-Py) |
| Z-2497 | —CH₂CF₃ | —C(═O)(3-Py) | Z-2498 | —Pr | —C(═O)(3-Py) | Z-2499 | —c-Pr | —C(═O)(3-Py) |
| Z-2500 | —CH₂CF₃ | —C(═O)(4-Py) | Z-2501 | —Pr | —C(═O)(4-Py) | Z-2502 | —c-Pr | —C(═O)(4-Py) |
| Z-2503 | —CH₂CF₃ | —C(═O)CF₂Me | Z-2504 | —Pr | —C(═O)CF₂Me | Z-2505 | —c-Pr | —C(═O)CF₂Me |
| Z-2506 | —CH₂CF₃ | —C(═O)NMe2 | Z-2507 | —Pr | —C(═O)NMe2 | Z-2508 | —c-Pr | —C(═O)NMe2 |
| Z-2509 | —CH₂CF₃ | —C(═O)—(1-CF₃—c-Pr) | Z-2510 | —Pr | —C(═O)—(1-CF₃—c-Pr) | Z-2511 | —c-Pr | —C(═O)—(1-CF₃—c-Pr) |
| Z-2512 | —CH₂CF₃ | —C(═O)—(1-F—c-Pr) | Z-2513 | —Pr | —C(═O)—(1-F—c-Pr) | Z-2514 | —c-Pr | —C(═O)—(1-F—c-Pr) |
| Z-2515 | —CH₂CF₃ | —SO₂Me | Z-2516 | —Pr | —SO₂Me | Z-2517 | —c-Pr | —SO₂Me |
| Z-2518 | —CH₂CF₃ | —C(═O)CF═CH₂ | Z-2519 | —Pr | —C(═O)CF═CH₂ | Z-2520 | —c-Pr | —C(═O)CF═CH₂ |
| Z-2521 | —CH₂CF₃ | —C(═O)—(4-Cl—Ph) | Z-2522 | —Pr | —C(═O)—(4-Cl—Ph) | Z-2523 | —c-Pr | —C(═O)—(4-Cl—Ph) |
| Z-2524 | —CH₂CF₃ | —C(═O)—(3-Cl—Ph) | Z-2525 | —Pr | —C(═O)—(3-Cl—Ph) | Z-2526 | —c-Pr | —C(═O)—(3-Cl—Ph) |
| Z-2527 | —CH₂CF₃ | —C(═O)—(3-CF₃—Ph) | Z-2528 | —Pr | —C(═O)—(3-CF₃—Ph) | Z-2529 | —c-Pr | —C(═O)—(3-CF₃—Ph) |
| Z-2530 | —CH₂CF₃ | —C(═O)—(2-Cl—Ph) | Z-2531 | —Pr | —C(═O)—(2-Cl—Ph) | Z-2532 | —c-Pr | —C(═O)—(2-Cl—Ph) |
| Z-2533 | —CH₂CF₃ | —C(═O)—(2-CF₃—Ph) | Z-2534 | —Pr | —C(═O)—(2-CF₃—Ph) | Z-2535 | —c-Pr | —C(═O)—(2-CF₃—Ph) |
| Z-2536 | —CH₂CF₃ | —C(═O)—(4-CF₃—Ph) | Z-2537 | —Pr | —C(═O)—(4-CF₃—Ph) | Z-2538 | —c-Pr | —C(═O)—(4-CF₃—Ph) |
| Z-2539 | —CH₂CF₃ | —C(═O)—(3-F—Ph) | Z-2540 | —Pr | —C(═O)—(3-F—Ph) | Z-2541 | —c-Pr | —C(═O)—(3-F—Ph) |
| Z-2542 | —CH₂CF₃ | —C(═O)—(4-F—Ph) | Z-2543 | —Pr | —C(═O)—(4-F—Ph) | Z-2544 | —c-Pr | —C(═O)—(4-F—Ph) |
| Z-2545 | —CH₂CF₃ | —C(═O)—(2-F—Ph) | Z-2546 | —Pr | —C(═O)—(2-F—Ph) | Z-2547 | —c-Pr | —C(═O)—(2-F—Ph) |
| Z-2548 | —CH₂CF₃ | —C(═O)—(4-OCF₃—Ph) | Z-2549 | —Pr | —C(═O)—(4-OCF₃—Ph) | Z-2550 | —c-Pr | —C(═O)—(4-OCF₃—Ph) |
| Z-2551 | —CH₂CF₃ | —C(═O)—(6-Cl—3-Py) | Z-2552 | —Pr | —C(═O)—(6-Cl—3-Py) | Z-2553 | —c-Pr | —C(═O)—(6-Cl—3-Py) |
| Z-2554 | —CH₂CF₃ | —C(═O)—(6-CF3—2-Py) | Z-2555 | —Pr | —C(═O)—(6-CF3—2-Py) | Z-2556 | —c-Pr | —C(═O)—(6-CF3—2-Py) |
| Z-2557 | —CH₂CF₃ | —C(═O)—(1-CN—c-Pr) | Z-2558 | —Pr | —C(═O)—(1-CN—c-Pr) | Z-2559 | —c-Pr | —C(═O)—(1-CN—c-Pr) |
| Z-2560 | —CH₂CF₃ | —C(═O)—(3-Cl—2-Py) | Z-2561 | —Pr | —C(═O)—(3-Cl—2-Py) | Z-2562 | —c-Pr | —C(═O)—(3-Cl—2-Py) |
| Z-2563 | —CH₂CF₃ | —C(═O)—(2-Me—Ph) | Z-2564 | —Pr | —C(═O)—(2-Me—Ph) | Z-2565 | —c-Pr | —C(═O)—(2-Me—Ph) |
| Z-2566 | —CH₂CF₃ | —C(═O)—(3-Me—Ph) | Z-2567 | —Pr | —C(═O)—(3-Me—Ph) | Z-2568 | —c-Pr | —C(═O)—(3-Me—Ph) |
| Z-2569 | —CH₂CF₃ | —C(═O)—(4-Me—Ph) | Z-2570 | —Pr | —C(═O)—(4-Me—Ph) | Z-2571 | —c-Pr | —C(═O)—(4-Me—Ph) |
| Z-2572 | —CH₂CF₃ | —C(═O)—(2-MeO—Ph) | Z-2573 | —Pr | —C(═O)—(2-MeO—Ph) | Z-2574 | —c-Pr | —C(═O)—(2-MeO—Ph) |
| Z-2575 | —CH₂CF₃ | —C(═O)—(3-MeO—Ph) | Z-2576 | —Pr | —C(═O)—(3-MeO—Ph) | Z-2577 | —c-Pr | —C(═O)—(3-MeO—Ph) |
| Z-2578 | —CH₂CF₃ | —C(═O)—(4-MeO—Ph) | Z-2579 | —Pr | —C(═O)—(4-MeO—Ph) | Z-2580 | —c-Pr | —C(═O)—(4-MeO—Ph) |
| Z-2581 | —CH₂CF₃ | —C(═O)—(4-Cl—2-Py) | 2-2582 | —Pr | —C(═O)—(4-Cl—2-Py) | Z-2583 | —c-Pr | —C(═O)—(4-Cl—2-Py) |
| Z-2584 | —CH₂CF₃ | —C(═O)—(5-Cl—2-Py) | Z-2585 | —Pr | —C(═O)—(5-Cl—2-Py) | Z-2586 | —c-Pr | —C(═O)—(5-Cl—2-Py) |
| Z-2587 | —CH₂CF₃ | —C(═O)—(6-Cl—2-Py) | Z-2588 | —Pr | —C(═O)—(6-Cl—2-Py) | Z-2589 | —c-Pr | —C(═O)—(6-Cl—2-Py) |
| Z-2590 | —CH₂CF₃ | —C(═O)—(2-Cl—3-Py) | Z-2591 | —Pr | —C(═O)—(2-Cl—3-Py) | Z-2592 | —c-Pr | —C(═O)—(2-Cl—3-Py) |
| Z-2593 | —CH₂CF₃ | —C(═O)—(2-Cl—4-Py) | Z-2594 | —Pr | —C(═O)—(2-Cl—4-Py) | Z-2595 | —c-Pr | —C(═O)—(2-Cl—4-Py) |
| Z-2596 | —CH₂CF₃ | —C(═O)—(3-Cl—4-Py) | Z-2597 | —Pr | —C(═O)—(3-Cl—4-Py) | Z-2598 | —c-Pr | —C(═O)—(3-Cl—4-Py) |
| Z-2599 | —CH₂CF₃ | —C(═O)—(3,4-di-Me—Ph) | Z-2600 | —Pr | —C(═O)—(3,4-di-Me—Ph) | Z-2601 | —c-Pr | —C(═O)—(3,4-di-Me—Ph) |
| Z-2602 | —CH₂CF₃ | —C(═O)—(3,5-di-Me—Ph) | Z-2603 | —Pr | —C(═O)—(3,5-di-Me—Ph) | Z-2604 | —c-Pr | —C(═O)—(3,5-di-Me—Ph) |
| Z-2605 | —CH₂CF₃ | —C(═O)—(4-Cl—3-Py) | Z-2606 | —Pr | —C(═O)—(4-Cl—3-Py) | Z-2607 | —c-Pr | —C(═O)—(4-Cl—3-Py) |
| Z-2608 | —CH₂CF₃ | —C(═O)—(5-Cl—3-Py) | Z-2609 | —Pr | —C(═O)—(5-Cl—3-Py) | Z-2610 | —c-Pr | —C(═O)—(5-Cl—3-Py) |
| Z-2611 | —CH₂CF₃ | —C(═O)—(4-Pyrimidine) | Z-2612 | —Pr | —C(═O)—(4-Pyrimidine) | Z-2613 | —c-Pr | —C(═O)—(4-Pyrimidine) |
| Z-2614 | —CH₂CF₃ | —C(═O)—(2-Cl—4-Pyrimidine) | Z-2615 | —Pr | —C(═O)—(2-Cl—4-Pyrimidine) | Z-2616 | —c-Pr | —C(═O)—(2-Cl—4-Pyrimidine) |
| Z-2617 | —CH₂CF₃ | —C(═O)—(4-Et—Ph) | Z-2618 | —Pr | —C(═O)—(4-Et—Ph) | Z-2619 | —c-Pr | —C(═O)—(4-Et—Ph) |
| Z-2620 | —CH₂CF₃ | —C(═O)—(2-Me—4-Pyrimidine) | Z-2621 | —Pr | —C(═O)—(2-Me—4-Pyrimidine) | Z-2622 | —c-Pr | —C(═O)—(2-Me—4-Pyrimidine) |
| Z-2623 | —CH₂CF₃ | —C(═O)—(6-Me—4-Pyrimidine) | Z-2624 | —Pr | —C(═O)—(6-Me—4-Pyrimidine) | Z-2625 | —c-Pr | —C(═O)—(6-Me—4-Pyrimidine) |
| Z-2626 | —CH₂CF₃ | —C(═O)—(6-Me—2-Py) | Z-2627 | —Pr | —C(═O)—(6-Me—2-Py) | Z-2628 | —c-Pr | —C(═O)—(6-Me—2-Py) |
| Z-2629 | —CH₂CF₃ | —C(═O)—(2-CF₃—4-Pyrimidine) | Z-2630 | —Pr | —C(═O)—(2-CF₃—4-Pyrimidine) | Z-2631 | —c-Pr | —C(═O)—(2-CF₃—4-Pyrimidine) |
| Z-2632 | —CH₂CF₃ | —C(═O)—(3-Pyridazine) | Z-2633 | —Pr | —C(═O)—(3-Pyridazine) | Z-2634 | —c-Pr | —C(═O)—(3-Pyridazine) |
| Z-2635 | —CH₂CF₃ | —C(═O)—(1-Me—c-Pr) | Z-2636 | —Pr | —C(═O)—(1-Me—c-Pr) | Z-2637 | —c-Pr | —C(═O)—(1-Me—c-Pr) |
| Z-2638 | —CH₂CF₃ | —C(═O)—(1-CF₃—c-Bu) | Z-2639 | —Pr | —C(═O)—(1-CF₃—c-Bu) | Z-2640 | —c-Pr | —C(═O)—(1-CF₃—c-Bu) |
| Z-2641 | —CH₂CF₃ | —C(═O)—(2-Pyrimidine) | Z-2642 | —Pr | —C(═O)—(2-Pyrimidine) | Z-2643 | —c-Pr | —C(═O)—(2-Pyrimidine) |
| Z-2644 | —CH₂CF₃ | —C(═O)—(2-Pyrazine) | Z-2645 | —Pr | —C(═O)—(2-Pyrazine) | Z-2646 | —c-Pr | —C(═O)—(2-Pyrazine) |
| Z-2647 | —CH₂CF₃ | —C(═O)CH═CHOEt | Z-2648 | —Pr | —C(═O)CH═CHOEt | Z-2649 | —c-Pr | —C(═O)CH═CHOEt |
| Z-2650 | —CH₂CF₃ | —C(═O)CH₂CHCF₃CF₃ | Z-2651 | —Pr | —C(═O)CH₂CHCF₃CF₃ | Z-2652 | —c-Pr | —C(═O)CH₂CHCF₃CF₃ |
| Z-2653 | —CH₂CF₃ | —C(═O)CH₂—(c-Pr) | Z-2654 | —Pr | —C(═O)CH₂—(c-Pr) | Z-2655 | —c-Pr | —C(═O)CH₂—(c-Pr) |
| Z-2656 | —CH₂CF₃ | —C(═O)—(2,2,3,3-tetra-Me—c-Pr) | Z-2657 | —Pr | —C(═O)—(2,2,3,3-tetra-Me—c-Pr) | Z-2658 | —c-Pr | —C(═O)—(2,2,3,3-tetra-Me—c-Pr) |
| Z-2659 | —CH₂CF₃ | —C(═O)CMe₂CF₃ | Z-2660 | —Pr | —C(═O)CMe₂CF₃ | Z-2661 | —c-Pr | —C(═O)CMe₂CF₃ |
| Z-2662 | —CH₂CF₂CF₃ | —H | Z-2663 | —C(═O)Me | —H | Z-2664 | —C(═O)Et | —H |
| Z-2665 | —CH₂CF₂CF₃ | —Me | Z-2666 | —C(═O)Me | —Me | Z-2667 | —C(═O)Et | —Me |
| Z-2668 | —CH₂CF₂CF₃ | —Et | Z-2669 | —C(═O)Me | —Et | Z-2670 | —C(═O)Et | —Et |
| Z-2671 | —CH₂CF₂CF₃ | —Pr | Z-2672 | —C(═O)Me | —Pr | Z-2673 | —C(═O)Et | —Pr |
| Z-2674 | —CH₂CF₂CF₃ | —i-Pr | Z-2675 | —C(═O)Me | —i-Pr | Z-2676 | —C(═O)Et | —i-Pr |
| Z-2677 | —CH₂CF₂CF₃ | —Bu | Z-2678 | —C(═O)Me | —Bu | Z-2679 | —C(═O)Et | —Bu |
| Z-2680 | —CH₂CF₂CF₃ | —sec-Bu | Z-2681 | —C(═O)Me | —sec-Bu | Z-2682 | —C(═O)Et | —sec-Bu |
| Z-2683 | —CH₂CF₂CF₃ | —I-Bu | Z-2684 | —C(═O)Me | —I-Bu | Z-2685 | —C(═O)Et | —I-Bu |
| Z-2686 | —CH₂CF₂CF₃ | —t-Bu | Z-2687 | —C(═O)Me | —t-Bu | Z-2688 | —C(═O)Et | —t-Bu |
| Z-2689 | —CH₂CF₂CF₃ | —Pent | Z-2690 | —C(═O)Me | —Pent | Z-2691 | —C(═O)Et | —Pent |
| Z-2692 | —CH₂CF₂CF₃ | —Hex | Z-2693 | —C(═O)Me | —Hex | Z-2694 | —C(═O)Et | —Hex |
| Z-2695 | —CH₂CF₂CF₃ | —CH(CH₃)CH₂CH₂CH₃ | Z-2696 | —C(═O)Me | —CH(CH₃)CH₂CH₂CH₃ | Z-2697 | —C(═O)Et | —CH(CH₃)CH₂CH₂CH₃ |
| Z-2698 | —CH₂CF₂CF₃ | —CH(CH₃)CH(CH₃)₂ | Z-2699 | —C(═O)Me | —CH(CH₃)CH(CH₃)₂ | Z-2700 | —C(═O)Et | —CH(CH₃)CH(CH₃)₂ |
| Z-2701 | —CH₂CF₂CF₃ | —C(CH₃)₂CH₂CH₃ | Z-2702 | —C(═O)Me | —C(CH₃)₂CH₂CH₃ | Z-2703 | —C(═O)Et | —C(CH₃)₂CH₂CH₃ |
| Z-2704 | —CH₂CF₂CF₃ | —CH(CH₂CH₃)₂ | Z-2705 | —C(═O)Me | —CH(CH₂CH₃)₂ | Z-2706 | —C(═O)Et | —CH(CH₂CH₃)₂ |
| Z-2707 | —CH₂CF₂CF₃ | —CH₂CH₂CH(CH₃)₂ | Z-2708 | —C(═O)Me | —CH₂CH₂CH(CH₃)₂ | Z-2709 | —C(═O)Et | —CH₂CH₂CH(CH₃)₂ |
| Z-2710 | —CH₂CF₂CF₃ | —CH₂CH₂CH₂CH(CH₃)₂ | Z-2711 | —C(═O)Me | —CH₂CH₂CH₂CH(CH₃)₂ | Z-2712 | —C(═O)Et | —CH₂CH₂CH₂CH(CH₃)₂ |
| Z-2713 | —CH₂CF₂CF₃ | —CH₂CH₂CH(CH₃)CH₂CH₃ | Z-2714 | —C(═O)Me | —CH₂CH₂CH(CH₃)CH₂CH₃ | Z-2715 | —C(═O)Et | —CH₂CH₂CH(CH₃)CH₂CH₃ |
| Z-2716 | —CH₂CF₂CF₃ | —CH₂CH(CH₃)CH₂CH₂CH₃ | Z-2717 | —C(═O)Me | —CH₂CH(CH₃)CH₂CH₂CH₃ | Z-2718 | —C(═O)Et | —CH₂CH(CH₃)CH₂CH₂CH₃ |
| Z-2719 | —CH₂CF₂CF₃ | —CH(CH₃)CH₂CH₂CH₂CH₃ | Z-2720 | —C(═O)Me | —CH(CH₃)CH₂CH₂CH₂CH₃ | Z-2721 | —C(═O)Et | —CH(CH₃)CH₂CH₂CH₂CH₃ |
| Z-2722 | —CH₂CF₂CF₃ | —CH(CH3)CH₂CH(CH₃)₂ | Z-2723 | —C(═O)Me | —CH(CH3)CH₂CH(CH₃)₂ | Z-2724 | —C(═O)Et | —CH(CH3)CH₂CH(CH₃)₂ |
| Z-2725 | —CH₂CF₂CF₃ | —CH(CH₃)CH(CH₃)CH₂CH₃ | Z-2726 | —C(═O)Me | —CH(CH₃)CH(CH₃)CH₂CH₃ | Z-2727 | —C(═O)Et | —CH(CH₃)CH(CH₃)CH₂CH₃ |
| Z-2728 | —CH₂CF₂CF₃ | —C(CH₃)₂CH₂CH₂CH₃ | Z-2729 | —C(═O)Me | —C(CH₃)₂CH₂CH₂CH₃ | Z-2730 | —C(═O)Et | —C(CH₃)₂CH₂CH₂CH₃ |
| Z-2731 | —CH₂CF₂CF₃ | —CH(CH₃)C(CH₃)₃ | Z-2732 | —C(═O)Me | —CH(CH₃)C(CH₃)₃ | Z-2733 | —C(═O)Et | —CH(CH₃)C(CH₃)₃ |
| Z-2734 | —CH₂CF₂CF₃ | —C(CH₃)₂CH(CH₃)₂ | Z-2735 | —C(═O)Me | —C(CH₃)₂CH(CH₃)₂ | Z-2736 | —C(═O)Et | —C(CH₃)₂CH(CH₃)₂ |
| Z-2737 | —CH₂CF₂CF₃ | —CH₂CH₂C(CH₃)₃ | Z-2738 | —C(═O)Me | —CH₂CH₂C(CH₃)₃ | Z-2739 | —C(═O)Et | —CH₂CH₂C(CH₃)₃ |
| Z-2740 | —CH₂CF₂CF₃ | —CH₂CH(CH₃)CH(CH₃)₂ | Z-2741 | —C(═O)Me | —CH₂CH(CH₃)CH(CH₃)₂ | Z-2742 | —C(═O)Et | —CH₂CH(CH₃)CH(CH₃)₂ |
| Z-2743 | —CH₂CF₂CF₃ | —CH₂C(CH₃)₂CH₂CH₃ | Z-2744 | —C(═O)Me | —CH₂C(CH₃)₂CH₂CH₃ | Z-2745 | —C(═O)Et | —CH₂C(CH₃)₂CH₂CH₃ |
| Z-2746 | —CH₂CF₂CF₃ | —CFH₂ | Z-2747 | —C(═O)Me | —CFH₂ | Z-2748 | —C(═O)Et | —CFH₂ |
| Z-2749 | —CH₂CF₂CF₃ | —CF₂H | Z-2750 | —C(═O)Me | —CF₂H | Z-2751 | —C(═O)Et | —CF₂H |
| Z-2752 | —CH₂CF₂CF₃ | —CF₃ | Z-2753 | —C(═O)Me | —CF₃ | Z-2754 | —C(═O)Et | —CF₃ |
| Z-2755 | —CH₂CF₂CF₃ | —CH₂Cl | Z-2756 | —C(═O)Me | —CH₂Cl | Z-2757 | —C(═O)Et | —CH₂Cl |
| Z-2758 | —CH₂CF₂CF₃ | —CHCl₂ | Z-2759 | —C(═O)Me | —CHCl₂ | Z-2760 | —C(═O)Et | —CHCl₂ |
| Z-2761 | —CH₂CF₂CF₃ | —CCl₃ | Z-2762 | —C(═O)Me | —CCl₃ | Z-2763 | —C(═O)Et | —CCl₃ |
| Z-2764 | —CH₂CF₂CF₃ | —CF₂Cl | Z-2765 | —C(═O)Me | —CF₂Cl | Z-2766 | —C(═O)Et | —CF₂Cl |
| Z-2767 | —CH₂CF₂CF₃ | —CCl₂F | Z-2768 | —C(═O)Me | —CCl₂F | Z-2769 | —C(═O)Et | —CCl₂F |
| Z-2770 | —CH₂CF₂CF₃ | —CH₂Br | Z-2771 | —C(═O)Me | —CH₂Br | Z-2772 | —C(═O)Et | —CH₂Br |
| Z-2773 | —CH₂CF₂CF₃ | —CHBr₂ | Z-2774 | —C(═O)Me | —CHBr₂ | Z-2775 | —C(═O)Et | —CHBr₂ |
| Z-2776 | —CH₂CF₂CF₃ | —CBr₃ | Z-2777 | —C(═O)Me | —CBr₃ | Z-2778 | —C(═O)Et | —CBr₃ |
| Z-2779 | —CH₂CF₂CF₃ | —CH₂l | Z-2780 | —C(═O)Me | —CH₂l | Z-2781 | —C(═O)Et | —CH₂l |
| Z-2782 | —CH₂CF₂CF₃ | —CHl₂ | Z-2783 | —C(═O)Me | —CHl₂ | Z-2784 | —C(═O)Et | —CHl₂ |
| Z-2785 | —CH₂CF₂CF₃ | —CH₂CF₂H | Z-2786 | —C(═O)Me | —CH₂CF₂H | Z-2787 | —C(═O)Et | —CH₂CF₂H |
| Z-2788 | —CH₂CF₂CF₃ | —CH₂CF₃ | Z-2789 | —C(═O)Me | —CH₂CF₃ | Z-2790 | —C(═O)Et | —CH₂CF₃ |
| Z-2791 | —CH₂CF₂CF₃ | —CF₂CH₃ | Z-2792 | —C(═O)Me | —CF₂CH₃ | Z-2793 | —C(═O)Et | —CF₂CH₃ |
| Z-2794 | —CH₂CF₂CF₃ | —CH₂CH₂CF₂H | Z-2795 | —C(═O)Me | —CH₂CH₂CF₂H | Z-2796 | —C(═O)Et | —CH₂CH₂CF₂H |
| Z-2797 | —CH₂CF₂CF₃ | —CH₂CH₂CF₃ | Z-2798 | —C(═O)Me | —CH₂CH₂CF₃ | Z-2799 | —C(═O)Et | —CH₂CH₂CF₃ |
| Z-2800 | —CH₂CF₂CF₃ | —CH₂CH₂CH₂CF₂H | Z-2801 | —C(═O)Me | —CH₂CH₂CH₂CF₂H | Z-2802 | —C(═O)Et | —CH₂CH₂CH₂CF₂H |
| Z-2803 | —CH₂CF₂CF₃ | —CH₂CH₂CH₂CF₃ | Z-2804 | —C(═O)Me | —CH₂CH₂CH₂CF₃ | Z-2805 | —C(═O)Et | —CH₂CH₂CH₂CF₃ |
| Z-2806 | —CH₂CF₂CF₃ | —CF₂CF₂H | Z-2807 | —C(═O)Me | —CF₂CF₂H | Z-2808 | —C(═O)Et | —CF₂CF₂H |
| Z-2809 | —CH₂CF₂CF₃ | —CF₂CF₂Cl | Z-2810 | —C(═O)Me | —CF₂CF₂Cl | Z-2811 | —C(═O)Et | —CF₂CF₂Cl |
| Z-2812 | —CH₂CF₂CF₃ | —CF₂CF₃ | Z-2813 | —C(═O)Me | —CF₂CF₃ | Z-2814 | —C(═O)Et | —CF₂CF₃ |
| Z-2815 | —CH₂CF₂CF₃ | —CFHCF₃ | Z-2816 | —C(═O)Me | —CFHCF₃ | Z-2817 | —C(═O)Et | —CFHCF₃ |
| 2-2818 | —CH₂CF₂CF₃ | —CH₂CF₂CF₂H | Z-2819 | —C(═O)Me | —CH₂CF₂CF₂H | Z-2820 | —C(═O)Et | —CH₂CF₂CF₂H |
| Z-2821 | —CH₂CF₂CF₃ | —CH₂CF₂CF₃ | Z-2822 | —C(═O)Me | —CH₂CF₂CF₃ | Z-2823 | —C(═O)Et | —CH₂CF₂CF₃ |
| Z-2824 | —CH₂CF₂CF₃ | —CF₂CF₂CF₃ | Z-2825 | —C(═O)Me | —CF₂CF₂CF₃ | Z-2826 | —C(═O)Et | —CF₂CF₂CF₃ |
| Z-2827 | —CH₂CF₂CF₃ | —CH₂CF₂CF₂CF₃ | Z-2828 | —C(═O)Me | —CH₂CF₂CF₂CF₃ | Z-2829 | —C(═O)Et | —CH₂CF₂CF₂CF₃ |
| Z-2830 | —CH₂CF₂CF₃ | —CF₂CF₂CF₂CF₃ | Z-2831 | —C(═O)Me | —CF₂CF₂CF₂CF₃ | Z-2832 | —C(═O)Et | —CF₂CF₂CF₂CF₃ |
| Z-2833 | —CH₂CF₂CF₃ | —CH₂CF₂CF₂CF₂CF₃ | Z-2834 | —C(═O)Me | —CH₂CF₂CF₂CF₂CF₃ | Z-2835 | —C(═O)Et | —CH₂CF₂CF₂CF₂CF₃ |
| Z-2836 | —CH₂CF₂CF₃ | c-Pr | Z-2837 | —C(═O)Me | c-Pr | Z-2838 | —C(═O)Et | c-Pr |
| Z-2839 | —CH₂CF₂CF₃ | c-Bu | Z-2840 | —C(═O)Me | c-Bu | Z-2841 | —C(═O)Et | c-Bu |
| Z-2842 | —CH₂CF₂CF₃ | c-Pent | Z-2843 | —C(═O)Me | c-Pent | Z-2844 | —C(═O)Et | c-Pent |
| Z-2845 | —CH₂CF₂CF₃ | c-Hex | Z-2846 | —C(═O)Me | c-Hex | Z-2847 | —C(═O)Et | c-Hex |
| Z-2848 | —CH₂CF₂CF₃ | c-Hept | Z-2849 | —C(═O)Me | c-Hept | Z-2850 | —C(═O)Et | c-Hept |
| Z-2851 | —CH₂CF₂CF₃ | c-Oct | Z-2852 | —C(═O)Me | c-Oct | Z-2853 | —C(═O)Et | c-Oct |
| Z-2854 | —CH₂CF₂CF₃ | —CH═CH₂ | Z-2855 | —C(═O)Me | —CH═CH₂ | Z-2856 | —C(═O)Et | —CH═CH₂ |
| Z-2857 | —CH₂CF₂CF₃ | —CH₂CH═CH₂ | Z-2858 | —C(═O)Me | —CH₂CH═CH₂ | Z-2859 | —C(═O)Et | —CH₂CH═CH₂ |
| Z-2860 | —CH₂CF₂CF₃ | —CH═CHCH₃ | Z-2861 | —C(═O)Me | —CH═CHCH₃ | Z-2862 | —C(═O)Et | —CH═CHCH₃ |
| Z-2863 | —CH₂CF₂CF₃ | —CH₂C(CH₃)═CH₂ | Z-2864 | —C(═O)Me | —CH₂C(CH₃)═CH₂ | Z-2865 | —C(═O)Et | —CH₂C(CH₃)═CH₂ |
| Z-2866 | —CH₂CF₂CF₃ | —CH₂CH₂CH═CH₂ | Z-2867 | —C(═O)Me | —CH₂CH₂CH═CH₂ | Z-2868 | —C(═O)Et | —CH₂CH₂CH═CH₂ |
| Z-2869 | —CH₂CF₂CF₃ | —CH₂CH═CHCH₃ | Z-2870 | —C(═O)Me | —CH₂CH═CHCH₃ | Z-2871 | —C(═O)Et | —CH₂CH═CHCH₃ |
| Z-2872 | —CH₂CF₂CF₃ | —CH═CHCH₂CH₃ | Z-2873 | —C(═O)Me | —CH═CHCH₂CH₃ | Z-2874 | —C(═O)Et | —CH═CHCH₂CH₃ |
| Z-2875 | —CH₂CF₂CF₃ | —CH₂CH═C(CH₃)₂ | Z-2876 | —C(═O)Me | —CH₂CH═C(CH₃)₂ | Z-2877 | —C(═O)Et | —CH₂CH═C(CH₃)₂ |
| Z-2878 | —CH₂CF₂CF₃ | —CH₂CH₂CH═C(CH₃)₂ | Z-2879 | —C(═O)Me | —CH₂CH₂CH═C(CH₃)₂ | Z-2880 | —C(═O)Et | —CH₂CH₂CH═C(CH₃)₂ |
| Z-2881 | —CH₂CF₂CF₃ | —CH═CFH | Z-2882 | —C(═O)Me | —CH═CFH | Z-2883 | —C(═O)Et | —CH═CFH |
| Z-2884 | —CH₂CF₂CF₃ | —CH═CF₂ | Z-2885 | —C(═O)Me | —CH═CF₂ | Z-2886 | —C(═O)Et | —CH═CF₂ |
| Z-2887 | —CH₂CF₂CF₃ | —CH═CCl₂ | Z-2888 | —C(═O)Me | —CH═CCl₂ | Z-2889 | —C(═O)Et | —CH═CCl₂ |
| Z-2890 | —CH₂CF₂CF₃ | —CH₂CH═CFH | Z-2891 | —C(═O)Me | —CH₂CH═CFH | Z-2892 | —C(═O)Et | —CH₂CH═CFH |
| Z-2893 | —CH₂CF₂CF₃ | —CH₂CH═CF₂ | Z-2894 | —C(═O)Me | —CH₂CH═CF₂ | Z-2895 | —C(═O)Et | —CH₂CH═CF₂ |
| Z-2896 | —CH₂CF₂CF₃ | —CH₂CH═CCl₂ | Z-2897 | —C(═O)Me | —CH₂CH═CCl₂ | Z-2898 | —C(═O)Et | —CH₂CH═CCl₂ |
| Z-2899 | —CH₂CF₂CF₃ | —CH₂CH₂CH═CF₂ | Z-2900 | —C(═O)Me | —CH₂CH₂CH═CF₂ | Z-2901 | —C(═O)Et | —CH₂CH₂CH═CF₂ |
| Z-2902 | —CH₂CF₂CF₃ | —CH₂CH₂CH₂CH═CF₂ | Z-2903 | —C(═O)Me | —CH₂CH₂CH₂CH═CF₂ | Z-2904 | —C(═O)Et | —CH₂CH₂CH₂CH═CF₂ |
| Z-2905 | —CH₂CF₂CF₃ | —CH₂CH₂CH₂CH₂CH═CF₂ | Z-2906 | —C(═O)Me | —CH₂CH₂CH₂CH₂CH═CF₂ | Z-2907 | —C(═O)Et | —CH₂CH₂CH₂CH₂CH═CF₂ |
| Z-2908 | —CH₂CF₂CF₃ | —C≡CH | Z-2909 | —C(═O)Me | —C≡CH | Z-2910 | —C(═O)Et | —C≡CH |
| Z-2911 | —CH₂CF₂CF₃ | —C≡CCH₃ | Z-2912 | —C(═O)Me | —C≡CCH₃ | Z-2913 | —C(═O)Et | —C≡CCH₃ |
| Z-2914 | —CH₂CF₂CF₃ | —CH₂C≡CH | Z-2915 | —C(═O)Me | —CH₂C≡CH | Z-2916 | —C(═O)Et | —CH₂C≡CH |
| Z-2917 | —CH₂CF₂CF₃ | —C≡CCH₂CH₃ | Z-2918 | —C(═O)Me | —C≡CCH₂CH₃ | Z-2919 | —C(═O)Et | —C≡CCH₂CH₃ |
| Z-2920 | —CH₂CF₂CF₃ | —CH₂C≡CCH₃ | Z-2921 | —C(═O)Me | —CH₂C≡CCH₃ | Z-2922 | —C(═O)Et | —CH₂C≡CCH₃ |
| Z-2923 | —CH₂CF₂CF₃ | —CH₂CH₂C≡CH | Z-2924 | —C(═O)Me | —CH₂CH₂C≡CH | Z-2925 | —C(═O)Et | —CH₂CH₂C≡CH |
| Z-2926 | —CH₂CF₂CF₃ | —C≡CCH₂CH₂CH₃ | Z-2927 | —C(═O)Me | —C≡CCH₂CH₂CH₃ | Z-2928 | —C(═O)Et | —C≡CCH₂CH₂CH₃ |
| Z-2929 | —CH₂CF₂CF₃ | —CH₂C≡CCH₂CH₃ | Z-2930 | —C(═O)Me | —CH₂C≡CCH₂CH₃ | Z-2931 | —C(═O)Et | —CH₂C≡CCH₂CH₃ |
| Z-2932 | —CH₂CF₂CF₃ | —C(CH₃)₂C≡CH | Z-2933 | —C(═O)Me | —C(CH₃)₂C≡CH | Z-2934 | —C(═O)Et | —C(CH₃)₂C≡CH |
| Z-2935 | —CH₂CF₂CF₃ | —C≡CF | Z-2936 | —C(═O)Me | —C≡CF | Z-2937 | —C(═O)Et | —C≡CF |
| Z-2938 | —CH₂CF₂CF₃ | —C≡CCF₂H | Z-2939 | —C(═O)Me | —C≡CCF₂H | Z-2940 | —C(═O)Et | —C≡CCF₂H |
| Z-2941 | —CH₂CF₂CF₃ | —C≡CCF₃ | Z-2942 | —C(═O)Me | —C≡CCF₃ | Z-2943 | —C(═O)Et | —C≡CCF₃ |
| Z-2944 | —CH₂CF₂CF₃ | —C≡CCH₂CF₂H | Z-2945 | —C(═O)Me | —C≡CCH₂CF₂H | Z-2946 | —C(═O)Et | —C≡CCH₂CF₂H |
| Z-2947 | —CH₂CF₂CF₃ | —C≡CCH₂CF₃ | Z-2948 | —C(═O)Me | —C≡CCH₂CF₃ | Z-2949 | —C(═O)Et | —C≡CCH₂CF₃ |
| Z-2950 | —CH₂CF₂CF₃ | —CH₂C≡CHCF₂H | Z-2951 | —C(═O)Me | —CH₂C≡CHCF₂H | Z-2952 | —C(═O)Et | —CH₂C≡CHCF₂H |
| Z-2953 | —CH₂CF₂CF₃ | —CH₂C≡CCF₃ | Z-2954 | —C(═O)Me | —CH₂C≡CCF₃ | Z-2955 | —C(═O)Et | —CH₂C≡CCF₃ |
| Z-2956 | —CH₂CF₂CF₃ | —C(═O)NH₂ | Z-2957 | —C(═O)Me | —C(═O)NH₂ | Z-2958 | —C(═O)Et | —C(═O)NH₂ |
| Z-2959 | —CH₂CF₂CF₃ | —C(═O)NHMe | Z-2960 | —C(═O)Me | —C(═O)NHMe | Z-2961 | —C(═O)Et | —C(═O)NHMe |
| Z-2962 | —CH₂CF₂CF₃ | —C(═O)NHEt | Z-2963 | —C(═O)Me | —C(═O)NHEt | Z-2964 | —C(═O)Et | —C(═O)NHEt |
| Z-2965 | —CH₂CF₂CF₃ | —C(═O)NHPr | Z-2966 | —C(═O)Me | —C(═O)NHPr | Z-2967 | —C(═O)Et | —C(═O)NHPr |
| Z-2968 | —CH₂CF₂CF₃ | —C(═O)NH—i-Pr | Z-2969 | —C(═O)Me | —C(═O)NH—i-Pr | Z-2970 | —C(═O)Et | —C(═O)NH—i-Pr |
| Z-2971 | —CH₂CF₂CF₃ | —C(═O)NHBu | Z-2972 | —C(═O)Me | —C(═O)NHBu | Z-2973 | —C(═O)Et | —C(═O)NHBu |
| Z-2974 | —CH₂CF₂CF₃ | —C(═O)NH—sec-Bu | Z-2975 | —C(═O)Me | —C(═O)NH—sec-Bu | Z-2976 | —C(═O)Et | —C(═O)NH—sec-Bu |
| Z-2977 | —CH₂CF₂CF₃ | —C(═O)NH—i-Bu | Z-2978 | —C(═O)Me | —C(═O)NH—i-Bu | Z-2979 | —C(═O)Et | —C(═O)NH—i-Bu |
| Z-2980 | —CH₂CF₂CF₃ | —C(═O)NH—t-Bu | Z-2981 | —C(═O)Me | —C(═O)NH—t-Bu | Z-2982 | —C(═O)Et | —C(═O)NH—t-Bu |
| Z-2983 | —CH₂CF₂CF₃ | —C(═O)NHPent | Z-2984 | —C(═O)Me | —C(═O)NHPent | Z-2985 | —C(═O)Et | —C(═O)NHPent |
| Z-2986 | —CH₂CF₂CF₃ | —C(═O)NHHex | Z-2987 | —C(═O)Me | —C(═O)NHHex | Z-2988 | —C(═O)Et | —C(═O)NHHex |
| Z-2989 | —CH₂CF₂CF₃ | —C(═O)NHCH₂C≡N | Z-2990 | —C(═O)Me | —C(═O)NHCH₂C≡N | Z-2991 | —C(═O)Et | —C(═O)NHCH₂C≡N |
| Z-2992 | —CH₂CF₂CF₃ | —C(═O)NHCH₂—c-Pr | Z-2993 | —C(═O)Me | —C(═O)NHCH₂—c-Pr | Z-2994 | —C(═O)Et | —C(═O)NHCH₂—c-Pr |
| Z-2995 | —CH₂CF₂CF₃ | —C(═O)NHCH₂OMe | Z-2996 | —C(═O)Me | —C(═O)NHCH₂OMe | Z-2997 | —C(═O)Et | —C(═O)NHCH₂OMe |
| Z-2998 | —CH₂CF₂CF₃ | —C(═O)NHCH₂CH₂OMe | Z-2999 | —C(═O)Me | —C(═O)NHCH₂CH₂OMe | Z-3000 | —C(═O)Et | —C(═O)NHCH₂CH₂OMe |
| Z-3001 | —CH₂CF₂CF₃ | —C(═O)NHCFH₂ | Z-3002 | —C(═O)Me | —C(═O)NHCFH₂ | Z-3003 | —C(═O)Et | —C(═O)NHCFH₂ |
| Z-3004 | —CH₂CF₂CF₃ | —C(═O)NHCF₂H | Z-3005 | —C(═O)Me | —C(═O)NHCF₂H | Z-3006 | —C(═O)Et | —C(═O)NHCF₂H |
| Z-3007 | —CH₂CF₂CF₃ | —C(═O)NHCF₃ | Z-3008 | —C(═O)Me | —C(═O)NHCF₃ | Z-3009 | —C(═O)Et | —C(═O)NHCF₃ |
| Z-3010 | —CH₂CF₂CF₃ | —C(═O)NHCH₂Cl | Z-3011 | —C(═O)Me | —C(═O)NHCH₂Cl | Z-3012 | —C(═O)Et | —C(═O)NHCH₂Cl |
| Z-3013 | —CH₂CF₂CF₃ | —C(═O)NHCHCl₂ | Z-3014 | —C(═O)Me | —C(═O)NHCHCl₂ | Z-3015 | —C(═O)Et | —C(═O)NHCHCl₂ |
| Z-3016 | —CH₂CF₂CF₃ | —C(═O)NHCCl₃ | Z-3017 | —C(═O)Me | —C(═O)NHCCl₃ | Z-3018 | —C(═O)Et | —C(═O)NHCCl₃ |
| Z-3019 | —CH₂CF₂CF₃ | —C(═O)NHCH₂Br | Z-3020 | —C(═O)Me | —C(═O)NHCH₂Br | Z-3021 | —C(═O)Et | —C(═O)NHCH₂Br |
| Z-3022 | —CH₂CF₂CF₃ | —C(═O)NHCHBr₂ | Z-3023 | —C(═O)Me | —C(═O)NHCHBr₂ | Z-3024 | —C(═O)Et | —C(═O)NHCHBr₂ |
| Z-3025 | —CH₂CF₂CF₃ | —C(═O)NHCBr₃ | Z-3026 | —C(═O)Me | —C(═O)NHCBr₃ | Z-3027 | —C(═O)Et | —C(═O)NHCBr₃ |
| Z-3028 | —CH₂CF₂CF₃ | —C(═O)NHCH₂l | Z-3029 | —C(═O)Me | —C(═O)NHCH₂l | Z-3030 | —C(═O)Et | —C(═O)NHCH₂l |
| Z-3031 | —CH₂CF₂CF₃ | —C(═O)NHCHl₂ | Z-3032 | —C(═O)Me | —C(═O)NHCHl₂ | Z-3033 | —C(═O)Et | —C(═O)NHCHl₂ |
| Z-3034 | —CH₂CF₂CF₃ | —C(═O)NHCH₂CF₂H | Z-3035 | —C(═O)Me | —C(═O)NHCH₂CF₂H | Z-3036 | —C(═O)Et | —C(═O)NHCH₂CF₂H |
| Z-3037 | —CH₂CF₂CF₃ | —C(═O)NHCH₂CF₃ | Z-3038 | —C(═O)Me | —C(═O)NHCH₂CF₃ | Z-3039 | —C(═O)Et | —C(═O)NHCH₂CF₃ |
| Z-3040 | —CH₂CF₂CF₃ | —C(═O)NHCH₂CH₂CF₂H | Z-3041 | —C(═O)Me | —C(═O)NHCH₂CH₂CF₂H | Z-3042 | —C(═O)Et | —C(═O)NHCH₂CH₂CF₂H |
| Z-3043 | —CH₂CF₂CF₃ | —C(═O)NHCH₂CH₂CF₃ | Z-3044 | —C(═O)Me | —C(═O)NHCH₂CH₂CF₃ | Z-3045 | —C(═O)Et | —C(═O)NHCH₂CH₂CF₃ |
| Z-3046 | —CH₂CF₂CF₃ | —C(═O)NHCH₂CH₂CH₂CF₂H | Z-3047 | —C(═O)Me | —C(═O)NHCH₂CH₂CH₂CF₂H | Z-3048 | —C(═O)Et | —C(═O)NHCH₂CH₂CH₂CF₂H |
| Z-3049 | —CH₂CF₂CF₃ | —C(═O)NHCH₂CH₂CH₂CF₃ | Z-3050 | —C(═O)Me | —C(═O)NHCH₂CH₂CH₂CF₃ | Z-3051 | —C(═O)Et | —C(═O)NHCH₂CH₂CH₂CF₃ |
| Z-3052 | —CH₂CF₂CF₃ | —C(═O)NHCF₂CF₂H | Z-3053 | —C(═O)Me | —C(═O)NHCF₂CF₂H | Z-3054 | —C(═O)Et | —C(═O)NHCF₂CF₂H |
| Z-3055 | —CH₂CF₂CF₃ | —C(═O)NHCF₂CF₃ | Z-3056 | —C(═O)Me | —C(═O)NHCF₂CF₃ | Z-3057 | —C(═O)Et | —C(═O)NHCF₂CF₃ |
| Z-3058 | —CH₂CF₂CF₃ | —C(═O)NHCFHCF₃ | Z-3059 | —C(═O)Me | —C(═O)NHCFHCF₃ | Z-3060 | —C(═O)Et | —C(═O)NHCFHCF₃ |
| Z-3061 | —CH₂CF₂CF₃ | —C(═O)NHCH₂CF₂CF₂H | Z-3062 | —C(═O)Me | —C(═O)NHCH₂CF₂CF₂H | Z-3063 | —C(═O)Et | —C(═O)NHCH₂CF₂CF₂H |
| Z-3064 | —CH₂CF₂CF₃ | —C(═O)NHCH₂CF₂CF₃ | Z-3065 | —C(═O)Me | —C(═O)NHCH₂CF₂CF₃ | Z-3066 | —C(═O)Et | —C(═O)NHCH₂CF₂CF₃ |
| Z-3067 | —CH₂CF₂CF₃ | —C(═O)NHCF₂CF₂CF₃ | Z-3068 | —C(═O)Me | —C(═O)NHCF₂CF₂CF₃ | Z-3069 | —C(═O)Et | —C(═O)NHCF₂CF₂CF₃ |
| Z-3070 | —CH₂CF₂CF₃ | —C(═O)NHCH₂CF₂CF₂CF₃ | Z-3071 | —C(═O)Me | —C(═O)NHCH₂CF₂CF₂CF₃ | Z-3072 | —C(═O)Et | —C(═O)NHCH₂CF₂CF₂CF₃ |
| Z-3073 | —CH₂CF₂CF₃ | —C(═O)NHCF₂CF₂CF₂CF₃ | Z-3074 | —C(═O)Me | —C(═O)NHCF₂CF₂CF₂CF₃ | Z-3075 | —C(═O)Et | —C(═O)NHCF₂CF₂CF₂CF₃ |
| Z-3076 | —CH₂CF₂CF₃ | —C(═O)NHCH₂CF₂CF₂CF₂CF₃ | Z-3077 | —C(═O)Me | —C(═O)NHCH₂CF₂CF₂CF₂CF₃ | Z-3078 | —C(═O)Et | —C(═O)NHCH₂CF₂CF₂CF₂CF₃ |
| Z-3079 | —CH₂CF₂CF₃ | —C(═O)NH—c-Pr | Z-3080 | —C(═O)Me | —C(═O)NH—c-Pr | Z-3081 | —C(═O)Et | —C(═O)NH—c-Pr |
| Z-3082 | —CH₂CF₂CF₃ | —C(═O)NH—c-Bu | Z-3083 | —C(═O)Me | —C(═O)NH—c-Bu | Z-3084 | —C(═O)Et | —C(═O)NH—c-Bu |
| Z-3085 | —CH₂CF₂CF₃ | —C(═O)NH—c-Pent | Z-3086 | —C(═O)Me | —C(═O)NH—c-Pent | Z-3087 | —C(═O)Et | —C(═O)NH—c-Pent |
| Z-3088 | —CH₂CF₂CF₃ | —C(═O)NH—c-Hex | Z-3089 | —C(═O)Me | —C(═O)NH—c-Hex | Z-3090 | —C(═O)Et | —C(═O)NH—c-Hex |
| 2-3091 | —CH₂CF₂CF₃ | —C(═O)NH—c-Hept | Z-3092 | —C(═O)Me | —C(═O)NH—c-Hept | Z-3093 | —C(═O)Et | —C(═O)NH—c-Hept |
| Z-3094 | —CH₂CF₂CF₃ | —C(═O)NH—c-Oct | Z-3095 | —C(═O)Me | —C(═O)NH—c-Oct | Z-3096 | —C(═O)Et | —C(═O)NH—c-Oct |
| Z-3097 | —CH₂CF₂CF₃ | —C(═O)NHCH₂CH═CH₂ | Z-3098 | —C(═O)Me | —C(═O)NHCH₂CH═CH₂ | Z-3099 | —C(═O)Et | —C(═O)NHCH₂CH═CH₂ |
| Z-3100 | —CH₂CF₂CF₃ | —C(═O)NHCH₂C(CH₃)═CH₂ | Z-3101 | —C(═O)Me | —C(═O)NHCH₂C(CH₃)═CH₂ | Z-3102 | —C(═O)Et | —C(═O)NHCH₂C(CH₃)═CH₂ |
| Z-3103 | —CH₂CF₂CF₃ | —C(═O)NHCH₂CH₂CH═CH₂ | Z-3104 | —C(═O)Me | —C(═O)NHCH₂CH₂CH═CH₂ | Z-3105 | —C(═O)Et | —C(═O)NHCH₂CH₂CH═CH₂ |
| Z-3106 | —CH₂CF₂CF₃ | —C(═O)NHCH₂CH═CHCH₃ | Z-3107 | —C(═O)Me | —C(═O)NHCH₂CH═CHCH₃ | Z-3108 | —C(═O)Et | —C(═O)NHCH₂CH═CHCH₃ |
| Z-3109 | —CH₂CF₂CF₃ | —C(═O)NHCH₂CH═C(CH₃)₂ | Z-3110 | —C(═O)Me | —C(═O)NHCH₂CH═C(CH₃)₂ | Z-3111 | —C(═O)Et | —C(═O)NHCH₂CH═C(CH₃)₂ |
| Z-3112 | —CH₂CF₂CF₃ | —C(═O)NHCH₂CH₂CH═C(CH₃)₂ | Z-3113 | —C(═O)Me | —C(═O)NHCH₂CH₂CH═C(CH₃)₂ | Z-3114 | —C(═O)Et | —C(═O)NHCH₂CH₂CH═C(CH₃)₂ |
| Z-3115 | —CH₂CF₂CF₃ | —C(═O)NHCH₂CH═CFH | Z-3116 | —C(═O)Me | —C(═O)NHCH₂CH═CFH | Z-3117 | —C(═O)Et | —C(═O)NHCH₂CH═CFH |
| Z-3118 | —CH₂CF₂CF₃ | —C(═O)NHCH₂CH═CF₂ | Z-3119 | —C(═O)Me | —C(═O)NHCH₂CH═CF₂ | Z-3120 | —C(═O)Et | —C(═O)NHCH₂CH═CF₂ |
| Z-3121 | —CH₂CF₂CF₃ | —C(═O)NHCH₂CH═CCl₂ | Z-3122 | —C(═O)Me | —C(═O)NHCH₂CH═CCl₂ | Z-3123 | —C(═O)Et | —C(═O)NHCH₂CH═CCl₂ |
| Z-3124 | —CH₂CF₂CF₃ | —C(═O)NHCH₂CH₂CH═CF₂ | Z-3125 | —C(═O)Me | —C(═O)NHCH₂CH₂CH═CF₂ | Z-3126 | —C(═O)Et | —C(═O)NHCH₂CH₂CH═CF₂ |
| Z-3127 | —CH₂CF₂CF₃ | —C(═O)NHCH₂CH₂CH₂CH═CF₂ | Z-3128 | —C(═O)Me | —C(═O)NHCH₂CH₂CH₂CH═CF₂ | Z-3129 | —C(═O)Et | —C(═O)NHCH₂CH₂CH₂CH═CF₂ |
| 2-3130 | —CH₂CF₂CF₃ | —C(═O)NHCH₂CH₂CH₂CH₂CH═CF₂ | Z-3131 | —C(═O)Me | —C(═O)NHCH₂CH₂CH₂CH₂CH═CF₂ | Z-3132 | —C(═O)Et | —C(═O)NHCH₂CH₂CH₂CH₂CH═CF₂ |
| Z-3133 | —CH₂CF₂CF₃ | —C(═O)NHCH₂C≡CH | Z-3134 | —C(═O)Me | —C(═O)NHCH₂C≡CH | Z-3135 | —C(═O)Et | —C(═O)NHCH₂C≡CH |
| Z-3136 | —CH₂CF₂CF₃ | —C(═O)NHCH₂C≡CCH₃ | Z-3137 | —C(═O)Me | —C(═O)NHCH₂C≡CCH₃ | Z-3138 | —C(═O)Et | —C(═O)NHCH₂C≡CCH₃ |
| Z-3139 | —CH₂CF₂CF₃ | —C(═O)NHCH₂CH₂C≡CH | Z-3140 | —C(═O)Me | —C(═O)NHCH₂CH₂C≡CH | Z-3141 | —C(═O)Et | —C(═O)NHCH₂CH₂C≡CH |
| Z-3142 | —CH₂CF₂CF₃ | —C(═O)NHCH₂C≡CCH₂CH₃ | Z-3143 | —C(═O)Me | —C(═O)NHCH₂C≡CCH₂CH₃ | Z-3144 | —C(═O)Et | —C(═O)NHCH₂C≡CCH₂CH₃ |
| Z-3145 | —CH₂CF₂CF₃ | —C(═O)NHC(CH₃)₂C≡CH | Z-3146 | —C(═O)Me | —C(═O)NHC(CH₃)₂C≡CH | Z-3147 | —C(═O)Et | —C(═O)NHC(CH₃)₂C≡CH |
| Z-3148 | —CH₂CF₂CF₃ | —C(═O)NHCH₂C≡CHCF₂H | Z-3149 | —C(═O)Me | —C(═O)NHCH₂C≡CHCF₂H | Z-3150 | —C(═O)Et | —C(═O)NHCH₂C≡CHCF₂H |
| Z-3151 | —CH₂CF₂CF₃ | —C(═O)NHCH₂C≡CCF₃ | Z-3152 | —C(═O)Me | —C(═O)NHCH₂C≡CCF₃ | Z-3153 | —C(═O)Et | —C(═O)NHCH₂C≡CCF₃ |
| Z-3154 | —CH₂CF₂CF₃ | —C(═O)H | Z-3155 | —C(═O)Me | —C(═O)H | Z-3156 | —C(═O)Et | —C(═O)H |
| Z-3157 | —CH₂CF₂CF₃ | —C(═O)Me | Z-3158 | —C(═O)Me | —C(═O)Me | Z-3159 | —C(═O)Et | —C(═O)Me |
| Z-3160 | —CH₂CF₂CF₃ | —C(═O)Et | Z-3161 | —C(═O)Me | —C(═O)Et | Z-3162 | —C(═O)Et | —C(═O)Et |
| Z-3163 | —CH₂CF₂CF₃ | —C(═O)Pr | Z-3164 | —C(═O)Me | —C(═O)Pr | Z-3165 | —C(═O)Et | —C(═O)Pr |
| Z-3166 | —CH₂CF₂CF₃ | —C(═O)—i-Pr | Z-3167 | —C(═O)Me | —C(═O)—i-Pr | Z-3168 | —C(═O)Et | —C(═O)—i-Pr |
| Z-3169 | —CH₂CF₂CF₃ | —C(═O)Bu | Z-3170 | —C(═O)Me | —C(═O)Bu | Z-3171 | —C(═O)Et | —C(═O)Bu |
| Z-3172 | —CH₂CF₂CF₃ | —C(═O)—sec-Bu | Z-3173 | —C(═O)Me | —C(═O)—sec-Bu | Z-3174 | —C(═O)Et | —C(═O)—sec-Bu |
| Z-3175 | —CH₂CF₂CF₃ | —C(═O)—i-Bu | Z-3176 | —C(═O)Me | —C(═O)—i-Bu | Z-3177 | —C(═O)Et | —C(═O)—i-Bu |
| Z-3178 | —CH₂CF₂CF₃ | —C(═O)—t-Bu | Z-3179 | —C(═O)Me | —C(═O)—t-Bu | Z-3180 | —C(═O)Et | —C(═O)—t-Bu |
| Z-3181 | —CH₂CF₂CF₃ | —C(═O)Pent | Z-3182 | —C(═O)Me | —C(═O)Pent | Z-3183 | —C(═O)Et | —C(═O)Pent |
| Z-3184 | —CH₂CF₂CF₃ | —C(═O)Hex | Z-3185 | —C(═O)Me | —C(═O)Hex | Z-3186 | —C(═O)Et | —C(═O)Hex |
| Z-3187 | —CH₂CF₂CF₃ | —C(═O)CH(CH₃)CH₂CH₂CH₃ | Z-3188 | —C(═O)Me | —C(═O)CH(CH₃)CH₂CH₂CH₃ | Z-3189 | —C(═O)Et | —C(═O)CH(CH₃)CH₂CH₂CH₃ |
| Z-3190 | —CH₂CF₂CF₃ | —C(═O)CH(CH₃)CH(CH₃)₂ | Z-3191 | —C(═O)Me | —C(═O)CH(CH₃)CH(CH₃)₂ | Z-3192 | —C(═O)Et | —C(═O)CH(CH₃)CH(CH₃)₂ |
| Z-3193 | —CH₂CF₂CF₃ | —C(═O)C(CH₃)₂CH₂CH₃ | Z-3194 | —C(═O)Me | —C(═O)C(CH₃)₂CH₂CH₃ | Z-3195 | —C(═O)Et | —C(═O)C(CH₃)₂CH₂CH₃ |
| Z-3196 | —CH₂CF₂CF₃ | —C(═O)CH(CH₂CH₃)₂ | Z-3197 | —C(═O)Me | —C(═O)CH(CH₂CH₃)₂ | Z-3198 | —C(═O)Et | —C(═O)CH(CH₂CH₃)₂ |
| Z-3199 | —CH₂CF₂CF₃ | —C(═O)CH₂CH₂CH(CH₃)₂ | Z-3200 | —C(═O)Me | —C(═O)CH₂CH₂CH(CH₃)₂ | Z-3201 | —C(═O)Et | —C(═O)CH₂CH₂CH(CH₃)₂ |
| Z-3202 | —CH₂CF₂CF₃ | —C(═O)CH₂CH₂CH₂CH(CH₃)₂ | Z-3203 | —C(═O)Me | —C(═O)CH₂CH₂CH₂CH(CH₃)₂ | Z-3204 | —C(═O)Et | —C(═O)CH₂CH₂CH₂CH(CH₃)₂ |
| Z-3205 | —CH₂CF₂CF₃ | —C(═O)CH₂CH₂CH(CH₃)CH₂CH₃ | Z-3206 | —C(═O)Me | —C(═O)CH₂CH₂CH(CH₃)CH₂CH₃ | Z-3207 | —C(═O)Et | —C(═O)CH₂CH₂CH(CH₃)CH₂CH₃ |
| Z-3208 | —CH₂CF₂CF₃ | —C(═O)CH₂CH(CH₃)CH₂CH₂CH₃ | Z-3209 | —C(═O)Me | —C(═O)CH₂CH(CH₃)CH₂CH₂CH₃ | Z-3210 | —C(═O)Et | —C(═O)CH₂CH(CH₃)CH₂CH₂CH₃ |
| Z-3211 | —CH₂CF₂CF₃ | —C(═O)CH(CH₃)CH₂CH₂CH₂CH₃ | Z-3212 | —C(═O)Me | —C(═O)CH(CH₃)CH₂CH₂CH₂CH₃ | Z-3213 | —C(═O)Et | —C(═O)CH(CH₃)CH₂CH₂CH₂CH₃ |
| Z-3214 | —CH₂CF₂CF₃ | —C(═O)CH(CH₃)CH₂CH(CH₃)₂ | Z-3215 | —C(═O)Me | —C(═O)CH(CH₃)CH₂CH(CH₃)₂ | Z-3216 | —C(═O)Et | —C(═O)CH(CH₃)CH₂CH(CH₃)₂ |
| Z-3217 | —CH₂CF₂CF₃ | —C(═O)CH(CH₃)CH(CH₃)CH₂CH₃ | Z-3218 | —C(═O)Me | —C(═O)CH(CH₃)CH(CH₃)CH₂CH₃ | Z-3219 | —C(═O)Et | —C(═O)CH(CH₃)CH(CH₃)CH₂CH₃ |
| Z-3220 | —CH₂CF₂CF₃ | —C(═O)C(CH₃)₂CH₂CH₂CH₃ | Z-3221 | —C(═O)Me | —C(═O)C(CH₃)₂CH₂CH₂CH₃ | Z-3222 | —C(═O)Et | —C(═O)C(CH₃)₂CH₂CH₂CH₃ |
| Z-3223 | —CH₂CF₂CF₃ | —C(═O)CH(CH₃)C(CH₃)₃ | Z-3224 | —C(═O)Me | —C(═O)CH(CH₃)C(CH₃)₃ | Z-3225 | —C(═O)Et | —C(═O)CH(CH₃)C(CH₃)₃ |
| Z-3226 | —CH₂CF₂CF₃ | —C(═O)C(CH₃)₂CH(CH₃)₂ | Z-3227 | —C(═O)Me | —C(═O)C(CH₃)₂CH(CH₃)₂ | Z-3228 | —C(═O)Et | —C(═O)C(CH₃)₂CH(CH₃)₂ |
| Z-3229 | —CH₂CF₂CF₃ | —C(═O)CH₂CH₂C(CH₃) | Z-3230 | —C(═O)Me | —C(═O)CH₂CH₂C(CH₃) | Z-3231 | —C(═O)Et | —C(═O)CH₂CH₂C(CH₃) |
| Z-3232 | —CH₂CF₂CF₃ | —C(═O)CH₂CH(CH₃)CH(CH₃)₂ | Z-3233 | —C(═O)Me | —C(═O)CH₂CH(CH₃)CH(CH₃)₂ | Z-3234 | —C(═O)Et | —C(═O)CH₂CH(CH₃)CH(CH₃)₂ |
| Z-3235 | —CH₂CF₂CF₃ | —C(═O)CH₂C(CH₃)₂CH₂CH₃ | Z-3236 | —C(═O)Me | —C(═O)CH₂C(CH₃)₂CH₂CH₃ | Z-3237 | —C(═O)Et | —C(═O)CH₂C(CH₃)₂CH₂CH₃ |
| Z-3238 | —CH₂CF₂CF₃ | —C(═O)CFH₂ | Z-3239 | —C(═O)Me | —C(═O)CFH₂ | Z-3240 | —C(═O)Et | —C(═O)CFH₂ |
| Z-3241 | —CH₂CF₂CF₃ | —C(═O)CF₂H | Z-3242 | —C(═O)Me | —C(═O)CF₂H | Z-3243 | —C(═O)Et | —C(═O)CF₂H |
| Z-3244 | —CH₂CF₂CF₃ | —C(═O)CF₂Cl | Z-3245 | —C(═O)Me | —C(═O)CF₂Cl | Z-3246 | —C(═O)Et | —C(═O)CF₂Cl |
| Z-3247 | —CH₂CF₂CF₃ | —C(═O)CF₃ | Z-3248 | —C(═O)Me | —C(═O)CF₃ | Z-3249 | —C(═O)Et | —C(═O)CF₃ |
| Z-3250 | —CH₂CF₂CF₃ | —C(═O)CH₂Cl | Z-3251 | —C(═O)Me | —C(═O)CH₂Cl | Z-3252 | —C(═O)Et | —C(═O)CH₂Cl |
| Z-3253 | —CH₂CF₂CF₃ | —C(═O)CHCl₂ | Z-3254 | —C(═O)Me | —C(═O)CHCl₂ | Z-3255 | —C(═O)Et | —C(═O)CHCl₂ |
| Z-3256 | —CH₂CF₂CF₃ | —C(═O)CCl₂F | Z-3257 | —C(═O)Me | —C(═O)CCl₂F | Z-3258 | —C(═O)Et | —C(═O)CCl₂F |
| Z-3259 | —CH₂CF₂CF₃ | —C(═O)CCl₃ | Z-3260 | —C(═O)Me | —C(═O)CCl₃ | Z-3261 | —C(═O)Et | —C(═O)CCl₃ |
| Z-3262 | —CH₂CF₂CF₃ | —C(═O)CH₂Br | Z-3263 | —C(═O)Me | —C(═O)CH₂Br | Z-3264 | —C(═O)Et | —C(═O)CH₂Br |
| Z-3265 | —CH₂CF₂CF₃ | —C(═O)CHBr₂ | Z-3266 | —C(═O)Me | —C(═O)CHBr₂ | Z-3267 | —C(═O)Et | —C(═O)CHBr₂ |
| Z-3268 | —CH₂CF₂CF₃ | —C(═O)CBr₃ | Z-3269 | —C(═O)Me | —C(═O)CBr₃ | Z-3270 | —C(═O)Et | —C(═O)CBr₃ |
| Z-3271 | —CH₂CF₂CF₃ | —C(═O)CH₂l | Z-3272 | —C(═O)Me | —C(═O)CH₂l | Z-3273 | —C(═O)Et | —C(═O)CH₂l |
| Z-3274 | —CH₂CF₂CF₃ | —C(═O)CHl₂ | Z-3275 | —C(═O)Me | —C(═O)CHl₂ | Z-3276 | —C(═O)Et | —C(═O)CHl₂ |
| Z-3277 | —CH₂CF₂CF₃ | —C(═O)CH₂CF₂H | Z-3278 | —C(═O)Me | —C(═O)CH₂CF₂H | Z-3279 | —C(═O)Et | —C(═O)CH₂CF₂H |
| Z-3280 | —CH₂CF₂CF₃ | —C(═O)CH₂CF₃ | Z-3281 | —C(═O)Me | —C(═O)CH₂CF₃ | Z-3282 | —C(═O)Et | —C(═O)CH₂CF₃ |
| Z-3283 | —CH₂CF₂CF₃ | —C(═O)CH₂CH₂CF₂H | Z-3284 | —C(═O)Me | —C(═O)CH₂CH₂CF₂H | Z-3285 | —C(═O)Et | —C(═O)CH₂CH₂CF₂H |
| Z-3286 | —CH₂CF₂CF₃ | —C(═O)CH₂CH₂CF₃ | Z-3287 | —C(═O)Me | —C(═O)CH₂CH₂CF₃ | Z-3288 | —C(═O)Et | —C(═O)CH₂CH₂CF₃ |
| Z-3289 | —CH₂CF₂CF₃ | —C(═O)CH₂CH₂CH₂CF₂H | Z-3290 | —C(═O)Me | —C(═O)CH₂CH₂CH₂CF₂H | Z-3291 | —C(═O)Et | —C(═O)CH₂CH₂CH₂CF₂H |
| Z-3292 | —CH₂CF₂CF₃ | —C(═O)CH₂CH₂CH₂CF₃ | Z-3293 | —C(═O)Me | —C(═O)CH₂CH₂CH₂CF₃ | Z-3294 | —C(═O)Et | —C(═O)CH₂CH₂CH₂CF₃ |
| Z-3295 | —CH₂CF₂CF₃ | —C(═O)CF₂CH₃ | Z-3296 | —C(═O)Me | —C(═O)CF₂CH₃ | Z-3297 | —C(═O)Et | —C(═O)CF₂CH₃ |
| Z-3298 | —CH₂CF₂CF₃ | —C(═O)CF₂CF₂H | Z-3299 | —C(═O)Me | —C(═O)CF₂CF₂H | Z-3300 | —C(═O)Et | —C(═O)CF₂CF₂H |
| Z-3301 | —CH₂CF₂CF₃ | —C(═O)CF₂CF₃ | Z-3302 | —C(═O)Me | —C(═O)CF₂CF₃ | Z-3303 | —C(═O)Et | —C(═O)CF₂CF₃ |
| Z-3304 | —CH₂CF₂CF₃ | —C(═O)CF₂CClF₂ | Z-3305 | —C(═O)Me | —C(═O)CF₂CClF₂ | Z-3306 | —C(═O)Et | —C(═O)CF₂CClF₂ |
| Z-3307 | —CH₂CF₂CF₃ | —C(═O)CFHCF₃ | Z-3308 | —C(═O)Me | —C(═O)CFHCF₃ | Z-3309 | —C(═O)Et | —C(═O)CFHCF₃ |
| Z-3310 | —CH₂CF₂CF₃ | —C(═O)CH₂CF₂CF₂H | Z-3311 | —C(═O)Me | —C(═O)CH₂CF₂CF₂H | Z-3312 | —C(═O)Et | —C(═O)CH₂CF₂CF₂H |
| Z-3313 | —CH₂CF₂CF₃ | —C(═O)CH₂CF₂CF₃ | Z-3314 | —C(═O)Me | —C(═O)CH₂CF₂CF₃ | Z-3315 | —C(═O)Et | —C(═O)CH₂CF₂CF₃ |
| Z-3316 | —CH₂CF₂CF₃ | —C(═O)CF₂CF₂CF₃ | Z-3317 | —C(═O)Me | —C(═O)CF₂CF₂CF₃ | Z-3318 | —C(═O)Et | —C(═O)CF₂CF₂CF₃ |
| Z-3319 | —CH₂CF₂CF₃ | —C(═O)CH₂CF₂CF₂CF₃ | Z-3320 | —C(═O)Me | —C(═O)CH₂CF₂CF₂CF₃ | Z-3321 | —C(═O)Et | —C(═O)CH₂CF₂CF₂CF₃ |
| Z-3322 | —CH₂CF₂CF₃ | —C(═O)CF₂CF₂CF₂CF₃ | Z-3323 | —C(═O)Me | —C(═O)CF₂CF₂CF₂CF₃ | Z-3324 | —C(═O)Et | —C(═O)CF₂CF₂CF₂CF₃ |
| Z-3325 | —CH₂CF₂CF₃ | —C(═O)CH₂CF₂CF₂CF₂CF₃ | Z-3326 | —C(═O)Me | —C(═O)CH₂CF₂CF₂CF₂CF₃ | Z-3327 | —C(═O)Et | —C(═O)CH₂CF₂CF₂CF₂CF₃ |
| Z-3328 | —CH₂CF₂CF₃ | —C(═O)CF₂CF₂CF₂CF₂CF₃ | Z-3329 | —C(═O)Me | —C(═O)CF₂CF₂CF₂CF₂CF₃ | Z-3330 | —C(═O)Et | —C(═O)CF₂CF₂CF₂CF₂CF₃ |
| Z-3331 | —CH₂CF₂CF₃ | —C(═O)—c-Pr | Z-3332 | —C(═O)Me | —C(═O)—c-Pr | Z-3333 | —C(═O)Et | —C(═O)—c-Pr |
| Z-3334 | —CH₂CF₂CF₃ | —C(═O)—c-Bu | Z-3335 | —C(═O)Me | —C(═O)—c-Bu | Z-3336 | —C(═O)Et | —C(═O)—c-Bu |
| Z-3337 | —CH₂CF₂CF₃ | —C(═O)—c-Pent | Z-3338 | —C(═O)Me | —C(═O)—c-Pent | Z-3339 | —C(═O)Et | —C(═O)—c-Pent |
| Z-3340 | —CH₂CF₂CF₃ | —C(═O)—c-Hex | Z-3341 | —C(═O)Me | —C(═O)—c-Hex | Z-3342 | —C(═O)Et | —C(═O)—c-Hex |
| Z-3343 | —CH₂CF₂CF₃ | —C(═O)—c-Hept | Z-3344 | —C(═O)Me | —C(═O)—c-Hept | Z-3345 | —C(═O)Et | —C(═O)—c-Hept |
| Z-3346 | —CH₂CF₂CF₃ | —C(═O)—c-Oct | Z-3347 | —C(═O)Me | —C(═O)—c-Oct | Z-3348 | —C(═O)Et | —C(═O)—c-Oct |
| Z-3349 | —CH₂CF₂CF₃ | —C(═O)CH═CH₂ | Z-3350 | —C(═O)Me | —C(═O)CH═CH₂ | Z-3351 | —C(═O)Et | —C(═O)CH═CH₂ |
| Z-3352 | —CH₂CF₂CF₃ | —C(═O)CH₂CH═CH₂ | Z-3353 | —C(═O)Me | —C(═O)CH₂CH═CH₂ | Z-3354 | —C(═O)Et | —C(═O)CH₂CH═CH₂ |
| Z-3355 | —CH₂CF₂CF₃ | —C(═O)CH═CHCH₃ | Z-3356 | —C(═O)Me | —C(═O)CH═CHCH₃ | Z-3357 | —C(═O)Et | —C(═O)CH═CHCH₃ |
| Z-3358 | —CH₂CF₂CF₃ | —C(═O)CH₂C(CH₃)═CH₂ | Z-3359 | —C(═O)Me | —C(═O)CH₂C(CH₃)═CH₂ | Z-3360 | —C(═O)Et | —C(═O)CH₂C(CH₃)═CH₂ |
| Z-3361 | —CH₂CF₂CF₃ | —C(═O)CH₂CH₂CH═CH₂ | Z-3362 | —C(═O)Me | —C(═O)CH₂CH₂CH═CH₂ | Z-3363 | —C(═O)Et | —C(═O)CH₂CH₂CH═CH₂ |
| Z-3364 | —CH₂CF₂CF₃ | —C(═O)CH₂CH═CHCH₃ | Z-3365 | —C(═O)Me | —C(═O)CH₂CH═CHCH₃ | Z-3366 | —C(═O)Et | —C(═O)CH₂CH═CHCH₃ |
| Z-3367 | —CH₂CF₂CF₃ | —C(═O)CH═CHCH₂CH₃ | Z-3368 | —C(═O)Me | —C(═O)CH═CHCH₂CH₃ | Z-3369 | —C(═O)Et | —C(═O)CH═CHCH₂CH₃ |
| Z-3370 | —CH₂CF₂CF₃ | —C(═O)CH₂CH═C(CH₃)₂ | Z-3371 | —C(═O)Me | —C(═O)CH₂CH═C(CH₃)₂ | Z-3372 | —C(═O)Et | —C(═O)CH₂CH═C(CH₃)₂ |
| Z-3373 | —CH₂CF₂CF₃ | —C(═O)CH₂CH₂CH═C(CH₃)₂ | Z-3374 | —C(═O)Me | —C(═O)CH₂CH₂CH═C(CH₃)₂ | Z-3375 | —C(═O)Et | —C(═O)CH₂CH₂CH═C(CH₃)₂ |
| Z-3376 | —CH₂CF₂CF₃ | —C(═O)CH═CFH | Z-3377 | —C(═O)Me | —C(═O)CH═CFH | Z-3378 | —C(═O)Et | —C(═O)CH═CFH |
| Z-3379 | —CH₂CF₂CF₃ | —C(═O)CH═CF₂ | Z-3380 | —C(═O)Me | —C(═O)CH═CF₂ | Z-3381 | —C(═O)Et | —C(═O)CH═CF₂ |
| Z-3382 | —CH₂CF₂CF₃ | —C(═O)CH═CCl₂ | Z-3383 | —C(═O)Me | —C(═O)CH═CCl₂ | Z-3384 | —C(═O)Et | —C(═O)CH═CCl₂ |
| Z-3385 | —CH₂CF₂CF₃ | —C(═O)CH₂CH═CFH | Z-3386 | —C(═O)Me | —C(═O)CH₂CH═CFH | Z-3387 | —C(═O)Et | —C(═O)CH₂CH═CFH |
| Z-3388 | —CH₂CF₂CF₃ | —C(═O)CH₂CH═CF₂ | Z-3389 | —C(═O)Me | —C(═O)CH₂CH═CF₂ | Z-3390 | —C(═O)Et | —C(═O)CH₂CH═CF₂ |
| Z-3391 | —CH₂CF₂CF₃ | —C(═O)CH₂CH═CCl₂ | Z-3392 | —C(═O)Me | —C(═O)CH₂CH═CCl₂ | Z-3393 | —C(═O)Et | —C(═O)CH₂CH═CCl₂ |
| Z-3394 | —CH₂CF₂CF₃ | —C(═O)CH₂CH₂CH═CF₂ | Z-3395 | —C(═O)Me | —C(═O)CH₂CH₂CH═CF₂ | Z-3396 | —C(═O)Et | —C(═O)CH₂CH₂CH═CF₂ |
| Z-3397 | —CH₂CF₂CF₃ | —C(═O)CH₂CH₂CH₂CH═CF₂ | Z-3398 | —C(═O)Me | —C(═O)CH₂CH₂CH₂CH═CF₂ | Z-3399 | —C(═O)Et | —C(═O)CH₂CH₂CH₂CH═CF₂ |
| Z-3400 | —CH₂CF₂CF₃ | —C(═O)CH₂CH₂CH₂CH₂CH═CF₂ | Z-3401 | —C(═O)Me | —C(═O)CH₂CH₂CH₂CH₂CH═CF₂ | Z-3402 | —C(═O)Et | —C(═O)CH₂CH₂CH₂CH₂CH═CF₂ |
| Z-3403 | —CH₂CF₂CF₃ | —C(═O)C≡CH | Z-3404 | —C(═O)Me | —C(═O)C≡CH | Z-3405 | —C(═O)Et | —C(═O)C≡CH |
| Z-3406 | —CH₂CF₂CF₃ | —C(═O)C≡CCH₃ | Z-3407 | —C(═O)Me | —C(═O)C≡CCH₃ | Z-3408 | —C(═O)Et | —C(═O)C≡CCH₃ |
| Z-3409 | —CH₂CF₂CF₃ | —C(═O)CH₂C≡CH | Z-3410 | —C(═O)Me | —C(═O)CH₂C≡CH | Z-3411 | —C(═O)Et | —C(═O)CH₂C≡CH |
| Z-3412 | —CH₂CF₂CF₃ | —C(═O)C≡CCH₂CH₃ | Z-3413 | —C(═O)Me | —C(═O)C≡CCH₂CH₃ | Z-3414 | —C(═O)Et | —C(═O)C≡CCH₂CH₃ |
| Z-3415 | —CH₂CF₂CF₃ | —C(═O)CH₂C≡CCH₃ | Z-3416 | —C(═O)Me | —C(═O)CH₂C≡CCH₃ | Z-3417 | —C(═O)Et | —C(═O)CH₂C≡CCH₃ |
| Z-3418 | —CH₂CF₂CF₃ | —C(═O)CH₂CH₂C≡CH | Z-3419 | —C(═O)Me | —C(═O)CH₂CH₂C≡CH | Z-3420 | —C(═O)Et | —C(═O)CH₂CH₂C≡CH |
| Z-3421 | —CH₂CF₂CF₃ | —C(═O)C≡CCH₂CH₂CH₃ | Z-3422 | —C(═O)Me | —C(═O)C≡CCH₂CH₂CH₃ | Z-3423 | —C(═O)Et | —C(═O)C≡CCH₂CH₂CH₃ |
| Z-3424 | —CH₂CF₂CF₃ | —C(═O)CH₂C≡CCH₂CH₃ | Z-3425 | —C(═O)Me | —C(═O)CH₂C≡CCH₂CH₃ | Z-3426 | —C(═O)Et | —C(═O)CH₂C≡CCH₂CH₃ |
| Z-3427 | —CH₂CF₂CF₃ | —C(═O)C(CH₃)₂C≡CH | Z-3428 | —C(═O)Me | —C(═O)C(CH₃)₂C≡CH | Z-3429 | —C(═O)Et | —C(═O)C(CH₃)₂C≡CH |
| Z-3430 | —CH₂CF₂CF₃ | —C(═O)C≡CF | Z-3431 | —C(═O)Me | —C(═O)C≡CF | Z-3432 | —C(═O)Et | —C(═O)C≡CF |
| Z-3433 | —CH₂CF₂CF₃ | —C(═O)C≡CCF₂H | Z-3434 | —C(═O)Me | —C(═O)C≡CCF₂H | Z-3435 | —C(═O)Et | —C(═O)C≡CCF₂H |
| Z-3436 | —CH₂CF₂CF₃ | —C(═O)C≡CCF₃ | Z-3437 | —C(═O)Me | —C(═O)C≡CCF₃ | Z-3438 | —C(═O)Et | —C(═O)C≡CCF₃ |
| Z-3439 | —CH₂CF₂CF₃ | —C(═O)C≡CCH₂CF₂H | Z-3440 | —C(═O)Me | —C(═O)C≡CCH₂CF₂H | Z-3441 | —C(═O)Et | —C(═O)C≡CCH₂CF₂H |
| Z-3442 | —CH₂CF₂CF₃ | —C(═O)C≡CCH₂CF₃ | Z-3443 | —C(═O)Me | —C(═O)C≡CCH₂CF₃ | Z-3444 | —C(═O)Et | —C(═O)C≡CCH₂CF₃ |
| Z-3445 | —CH₂CF₂CF₃ | —C(═O)CH₂C≡CHCF₂H | Z-3446 | —C(═O)Me | —C(═O)CH₂C≡CHCF₂H | Z-3447 | —C(═O)Et | —C(═O)CH₂C≡CHCF₂H |
| Z-3448 | —CH₂CF₂CF₃ | —C(═O)CH₂C≡CCF₃ | Z-3449 | —C(═O)Me | —C(═O)CH₂C≡CCF₃ | Z-3450 | —C(═O)Et | —C(═O)CH₂C≡CCF₃ |
| Z-3451 | —CH₂CF₂CF₃ | —C(═O)CH₂C≡N | Z-3452 | —C(═O)Me | —C(═O)CH₂C≡N | Z-3453 | —C(═O)Et | —C(═O)CH₂C≡N |
| Z-3454 | —CH₂CF₂CF₃ | —C(═O)C(Me)C≡N | Z-3455 | —C(═O)Me | —C(═O)C(Me)C≡N | Z-3456 | —C(═O)Et | —C(═O)C(Me)C≡N |
| Z-3457 | —CH₂CF₂CF₃ | —C(═O)CH₂CH₂C≡N | Z-3458 | —C(═O)Me | —C(═O)CH₂CH₂C≡N | Z-3459 | —C(═O)Et | —C(═O)CH₂CH₂C≡N |
| Z-3460 | —CH₂CF₂CF₃ | —C(═O)CH₂CH₂CH₂C≡N | Z-3461 | —C(═O)Me | —C(═O)CH₂CH₂CH₂C≡N | Z-3462 | —C(═O)Et | —C(═O)CH₂CH₂CH₂C≡N |
| Z-3463 | —CH₂CF₂CF₃ | —C(═O)CH₂OH | Z-3464 | —C(═O)Me | —C(═O)CH₂OH | Z-3465 | —C(═O)Et | —C(═O)CH₂OH |
| Z-3466 | —CH₂CF₂CF₃ | —C(═O)CH₂OMe | Z-3467 | —C(═O)Me | —C(═O)CH₂OMe | Z-3468 | —C(═O)Et | —C(═O)CH₂OMe |
| Z-3469 | —CH₂CF₂CF₃ | —C(═O)CH₂OEt | Z-3470 | —C(═O)Me | —C(═O)CH₂OEt | Z-3471 | —C(═O)Et | —C(═O)CH₂OEt |
| Z-3472 | —CH₂CF₂CF₃ | —C(═O)CH₂OPr | Z-3473 | —C(═O)Me | —C(═O)CH₂OPr | Z-3474 | —C(═O)Et | —C(═O)CH₂OPr |
| Z-3475 | —CH₂CF₂CF₃ | —C(═O)CH₂CH₂OMe | Z-3476 | —C(═O)Me | —C(═O)CH₂CH₂OMe | Z-3477 | —C(═O)Et | —C(═O)CH₂CH₂OMe |
| Z-3478 | —CH₂CF₂CF₃ | —C(═O)CH₂CH₂OEt | Z-3479 | —C(═O)Me | —C(═O)CH₂CH₂OEt | Z-3480 | —C(═O)Et | —C(═O)CH₂CH₂OEt |
| Z-3481 | —CH₂CF₂CF₃ | —C(═O)CH₂—(1-Pyra) | Z-3482 | —C(═O)Me | —C(═O)CH₂—(1-Pyra) | Z-3483 | —C(═O)Et | —C(═O)CH₂—(1-Pyra) |
| Z-3484 | —CH₂CF₂CF₃ | —C(═O)CH₂—(1-Tria) | Z-3485 | —C(═O)Me | —C(═O)CH₂—(1-Tria) | Z-3486 | —C(═O)Et | —C(═O)CH₂—(1-Tria) |
| Z-3487 | —CH₂CF₂CF₃ | —C(═O)OH | Z-3488 | —C(═O)Me | —C(═O)OH | Z-3489 | —C(═O)Et | —C(═O)OH |
| Z-3490 | —CH₂CF₂CF₃ | —C(═O)OMe | Z-3491 | —C(═O)Me | —C(═O)OMe | Z-3492 | —C(═O)Et | —C(═O)OMe |
| Z-3493 | —CH₂CF₂CF₃ | —C(═O)OEt | Z-3494 | —C(═O)Me | —C(═O)OEt | Z-3495 | —C(═O)Et | —C(═O)OEt |
| Z-3496 | —CH₂CF₂CF₃ | —C(═O)OPr | Z-3497 | —C(═O)Me | —C(═O)OPr | Z-3498 | —C(═O)Et | —C(═O)OPr |
| Z-3499 | —CH₂CF₂CF₃ | —C(═O)O—i-Pr | Z-3500 | —C(═O)Me | —C(═O)O—i-Pr | Z-3501 | —C(═O)Et | —C(═O)O—i-Pr |
| Z-3502 | —CH₂CF₂CF₃ | —C(═O)OBu | Z-3503 | —C(═O)Me | —C(═O)OBu | Z-3504 | —C(═O)Et | —C(═O)OBu |
| Z-3505 | —CH₂CF₂CF₃ | —C(═O)O—sec-Bu | Z-3506 | —C(═O)Me | —C(═O)O—sec-Bu | Z-3507 | —C(═O)Et | —C(═O)O—sec-Bu |
| Z-3508 | —CH₂CF₂CF₃ | —C(═O)O—i-Bu | Z-3509 | —C(═O)Me | —C(═O)O—i-Bu | Z-3510 | —C(═O)Et | —C(═O)O—i-Bu |
| Z-3511 | —CH₂CF₂CF₃ | —C(═O)O—t-Bu | Z-3512 | —C(═O)Me | —C(═O)O—t-Bu | Z-3513 | —C(═O)Et | —C(═O)O—t-Bu |
| Z-3514 | —CH₂CF₂CF₃ | —C(═O)OPent | Z-3515 | —C(═O)Me | —C(═O)OPent | Z-3516 | —C(═O)Et | —C(═O)OPent |
| Z-3517 | —CH₂CF₂CF₃ | —C(═O)OHex | Z-3518 | —C(═O)Me | —C(═O)OHex | Z-3519 | —C(═O)Et | —C(═O)OHex |
| Z-3520 | —CH₂CF₂CF₃ | —C(═O)OCH(CH₃)CH₂CH₂CH₃ | Z-3521 | —C(═O)Me | —C(═O)OCH(CH₃)CH₂CH₂CH₃ | Z-3522 | —C(═O)Et | —C(═O)OCH(CH₃)CH₂CH₂CH₃ |
| Z-3523 | —CH₂CF₂CF₃ | —C(═O)OCH(CH₃)CH(CH₃)₂ | Z-3524 | —C(═O)Me | —C(═O)OCH(CH₃)CH(CH₃)₂ | Z-3525 | —C(═O)Et | —C(═O)OCH(CH₃)CH(CH₃)₂ |
| Z-3526 | —CH₂CF₂CF₃ | —C(═O)OC(CH₃)₂CH₂CH₃ | Z-3527 | —C(═O)Me | —C(═O)OC(CH₃)₂CH₂CH₃ | Z-3528 | —C(═O)Et | —C(═O)OC(CH₃)₂CH₂CH₃ |
| Z-3529 | —CH₂CF₂CF₃ | —C(═O)OCH(CH₂CH₃)₂ | Z-3530 | —C(═O)Me | —C(═O)OCH(CH₂CH₃)₂ | Z-3531 | —C(═O)Et | —C(═O)OCH(CH₂CH₃)₂ |
| Z-3532 | —CH₂CF₂CF₃ | —C(═O)OCH₂CH₂CH(CH₃)₂ | Z-3533 | —C(═O)Me | —C(═O)OCH₂CH₂CH(CH₃)₂ | Z-3534 | —C(═O)Et | —C(═O)OCH₂CH₂CH(CH₃)₂ |
| Z-3535 | —CH₂CF₂CF₃ | —C(═O)OCH₂CH₂CH₂CH(CH₃)₂ | Z-3536 | —C(═O)Me | —C(═O)OCH₂CH₂CH₂CH(CH₃)₂ | Z-3537 | —C(═O)Et | —C(═O)OCH₂CH₂CH₂CH(CH₃)₂ |
| Z-3538 | —CH₂CF₂CF₃ | —C(═O)OCH₂CH₂CH(CH₃)CH₂CH₃ | Z-3539 | —C(═O)Me | —C(═O)OCH₂CH₂CH(CH₃)CH₂CH₃ | Z-3540 | —C(═O)Et | —C(═O)OCH₂CH₂CH(CH₃)CH₂CH₃ |
| Z-3541 | —CH₂CF₂CF₃ | —C(═O)OCH₂CH(CH₃)CH₂CH₂CH₃ | Z-3542 | —C(═O)Me | —C(═O)OCH₂CH(CH₃)CH₂CH₂CH₃ | Z-3543 | —C(═O)Et | —C(═O)OCH₂CH(CH₃)CH₂CH₂CH₃ |
| Z-3544 | —CH₂CF₂CF₃ | —C(═O)OCH(CH₃)CH₂CH₂CH₂CH₃ | Z-3545 | —C(═O)Me | —C(═O)OCH(CH₃)CH₂CH₂CH₂CH₃ | Z-3546 | —C(═O)Et | —C(═O)OCH(CH₃)CH₂CH₂CH₂CH₃ |
| Z-3547 | —CH₂CF₂CF₃ | —C(═O)OCH(CH₃)CH₂CH(CH₃)₂ | Z-3548 | —C(═O)Me | —C(═O)OCH(CH₃)CH₂CH(CH₃)₂ | Z-3549 | —C(═O)Et | —C(═O)OCH(CH₃)CH₂CH(CH₃)₂ |
| Z-3550 | —CH₂CF₂CF₃ | —C(═O)OCH(CH₃)CH(CH₃)CH₂CH₃ | Z-3551 | —C(═O)Me | —C(═O)OCH(CH₃)CH(CH₃)CH₂CH₃ | Z-3552 | —C(═O)Et | —C(═O)OCH(CH₃)CH(CH₃)CH₂CH₃ |
| Z-3553 | —CH₂CF₂CF₃ | —C(═O)OC(CH₃)₂CH₂CH₂CH₃ | Z-3554 | —C(═O)Me | —C(═O)OC(CH₃)₂CH₂CH₂CH₃ | Z-3555 | —C(═O)Et | —C(═O)OC(CH₃)₂CH₂CH₂CH₃ |
| Z-3556 | —CH₂CF₂CF₃ | —C(═O)OCH(CH₃)C(CH₃)₃ | Z-3557 | —C(═O)Me | —C(═O)OCH(CH₃)C(CH₃)₃ | Z-3558 | —C(═O)Et | —C(═O)OCH(CH₃)C(CH₃)₃ |
| Z-3559 | —CH₂CF₂CF₃ | —C(═O)OC(CH₃)₂CH(CH₃)₂ | Z-3560 | —C(═O)Me | —C(═O)OC(CH₃)₂CH(CH₃)₂ | Z-3561 | —C(═O)Et | —C(═O)OC(CH₃)₂CH(CH₃)₂ |
| Z-3562 | —CH₂CF₂CF₃ | —C(═O)OCH₂CH₂C(CH₃)₃ | Z-3563 | —C(═O)Me | —C(═O)OCH₂CH₂C(CH₃)₃ | Z-3564 | —C(═O)Et | —C(═O)OCH₂CH₂C(CH₃)₃ |
| Z-3565 | —CH₂CF₂CF₃ | —C(═O)OCH₂CH(CH₃)CH(CH₃)₂ | Z-3566 | —C(═O)Me | —C(═O)OCH₂CH(CH₃)CH(CH₃)₂ | Z-3567 | —C(═O)Et | —C(═O)OCH₂CH(CH₃)CH(CH₃)₂ |
| Z-3568 | —CH₂CF₂CF₃ | —C(═O)OCH₂C(CH₃)₂CH₂CH₃ | Z-3569 | —C(═O)Me | —C(═O)OCH₂C(CH₃)₂CH₂CH₃ | Z-3570 | —C(═O)Et | —C(═O)OCH₂C(CH₃)₂CH₂CH₃ |
| Z-3571 | —CH₂CF₂CF₃ | —C(═O)OCFH₂ | Z-3572 | —C(═O)Me | —C(═O)OCFH₂ | Z-3573 | —C(═O)Et | —C(═O)OCFH₂ |
| Z-3574 | —CH₂CF₂CF₃ | —C(═O)OCF₂H | Z-3575 | —C(═O)Me | —C(═O)OCF₂H | Z-3576 | —C(═O)Et | —C(═O)OCF₂H |
| Z-3577 | —CH₂CF₂CF₃ | —C(═O)OCF₃ | Z-3578 | —C(═O)Me | —C(═O)OCF₃ | Z-3579 | —C(═O)Et | —C(═O)OCF₃ |
| Z-3580 | —CH₂CF₂CF₃ | —C(═O)OCH₂Cl | Z-3581 | —C(═O)Me | —C(═O)OCH₂Cl | Z-3582 | —C(═O)Et | —C(═O)OCH₂Cl |
| Z-3583 | —CH₂CF₂CF₃ | —C(═O)OCHCl₂ | Z-3584 | —C(═O)Me | —C(═O)OCHCl₂ | Z-3585 | —C(═O)Et | —C(═O)OCHCl₂ |
| Z-3586 | —CH₂CF₂CF₃ | —C(═O)OCCl₃ | Z-3587 | —C(═O)Me | —C(═O)OCCl₃ | Z-3588 | —C(═O)Et | —C(═O)OCCl₃ |
| Z-3589 | —CH₂CF₂CF₃ | —C(═O)OCH₂Br | Z-3590 | —C(═O)Me | —C(═O)OCH₂Br | Z-3591 | —C(═O)Et | —C(═O)OCH₂Br |
| Z-3592 | —CH₂CF₂CF₃ | —C(═O)OCHBr₂ | Z-3593 | —C(═O)Me | —C(═O)OCHBr₂ | Z-3594 | —C(═O)Et | —C(═O)OCHBr₂ |
| Z-3595 | —CH₂CF₂CF₃ | —C(═O)OCBr₃ | Z-3596 | —C(═O)Me | —C(═O)OCBr₃ | Z-3597 | —C(═O)Et | —C(═O)OCBr₃ |
| Z-3598 | —CH₂CF₂CF₃ | —C(═O)OCH₂l | Z-3599 | —C(═O)Me | —C(═O)OCH₂l | Z-3600 | —C(═O)Et | —C(═O)OCH₂l |
| Z-3601 | —CH₂CF₂CF₃ | —C(═O)OCHl₂ | Z-3602 | —C(═O)Me | —C(═O)OCHl₂ | Z-3603 | —C(═O)Et | —C(═O)OCHl₂ |
| Z-3604 | —CH₂CF₂CF₃ | —C(═O)OCH₂CF₂H | Z-3605 | —C(═O)Me | —C(═O)OCH₂CF₂H | Z-3606 | —C(═O)Et | —C(═O)OCH₂CF₂H |
| Z-3607 | —CH₂CF₂CF₃ | —C(═O)OCH₂CF₃ | Z-3608 | —C(═O)Me | —C(═O)OCH₂CF₃ | Z-3609 | —C(═O)Et | —C(═O)OCH₂CF₃ |
| Z-3610 | —CH₂CF₂CF₃ | —C(═O)OCH₂CH₂CF₂H | Z-3611 | —C(═O)Me | —C(═O)OCH₂CH₂CF₂H | Z-3612 | —C(═O)Et | —C(═O)OCH₂CH₂CF₂H |
| Z-3613 | —CH₂CF₂CF₃ | —C(═O)OCH₂CH₂CF₃ | Z-3614 | —C(═O)Me | —C(═O)OCH₂CH₂CF₃ | Z-3615 | —C(═O)Et | —C(═O)OCH₂CH₂CF₃ |
| Z-3616 | —CH₂CF₂CF₃ | —C(═O)OCH₂CH₂CH₂CF₂H | Z-3617 | —C(═O)Me | —C(═O)OCH₂CH₂CH₂CF₂H | Z-3618 | —C(═O)Et | —C(═O)OCH₂CH₂CH₂CF₂H |
| Z-3619 | —CH₂CF₂CF₃ | —C(═O)OCH₂CH₂CH₂CF₃ | Z-3620 | —C(═O)Me | —C(═O)OCH₂CH₂CH₂CF₃ | Z-3621 | —C(═O)Et | —C(═O)OCH₂CH₂CH₂CF₃ |
| Z-3622 | —CH₂CF₂CF₃ | —C(═O)OCF₂CF₂H | Z-3623 | —C(═O)Me | —C(═O)OCF₂CF₂H | Z-3624 | —C(═O)Et | —C(═O)OCF₂CF₂H |
| Z-3625 | —CH₂CF₂CF₃ | —C(═O)OCF₂CF₃ | Z-3626 | —C(═O)Me | —C(═O)OCF₂CF₃ | Z-3627 | —C(═O)Et | —C(═O)OCF₂CF₃ |
| Z-3628 | —CH₂CF₂CF₃ | —C(═O)OCFHCF₃ | Z-3629 | —C(═O)Me | —C(═O)OCFHCF₃ | Z-3630 | —C(═O)Et | —C(═O)OCFHCF₃ |
| Z-3631 | —CH₂CF₂CF₃ | —C(═O)OCH₂CF₂CF₂H | Z-3632 | —C(═O)Me | —C(═O)OCH₂CF₂CF₂H | Z-3633 | —C(═O)Et | —C(═O)OCH₂CF₂CF₂H |
| Z-3634 | —CH₂CF₂CF₃ | —C(═O)OCH₂CF₂CF₃ | Z-3635 | —C(═O)Me | —C(═O)OCH₂CF₂CF₃ | Z-3636 | —C(═O)Et | —C(═O)OCH₂CF₂CF₃ |
| Z-3637 | —CH₂CF₂CF₃ | —C(═O)OCF₂CF₂CF₃ | Z-3638 | —C(═O)Me | —C(═O)OCF₂CF₂CF₃ | Z-3639 | —C(═O)Et | —C(═O)OCF₂CF₂CF₃ |
| Z-3640 | —CH₂CF₂CF₃ | —C(═O)OCH₂CF₂CF₂CF₃ | Z-3641 | —C(═O)Me | —C(═O)OCH₂CF₂CF₂CF₃ | Z-3642 | —C(═O)Et | —C(═O)OCH₂CF₂CF₂CF₃ |
| Z-3643 | —CH₂CF₂CF₃ | —C(═O)OCF₂CF₂CF₂CF₃ | Z-3644 | —C(═O)Me | —C(═O)OCF₂CF₂CF₂CF₃ | Z-3645 | —C(═O)Et | —C(═O)OCF₂CF₂CF₂CF₃ |
| Z-3646 | —CH₂CF₂CF₃ | —C(═O)OCH₂CF₂CF₂CF₂CF₃ | Z-3647 | —C(═O)Me | —C(═O)OCH₂CF₂CF₂CF₂CF₃ | Z-3648 | —C(═O)Et | —C(═O)OCH₂CF₂CF₂CF₂CF₃ |
| Z-3649 | —CH₂CF₂CF₃ | —C(═O)O—c-Pr | Z-3650 | —C(═O)Me | —C(═O)O—c-Pr | Z-3651 | —C(═O)Et | —C(═O)O—c-Pr |
| Z-3652 | —CH₂CF₂CF₃ | —C(═O)O—c-Bu | Z-3653 | —C(═O)Me | —C(═O)O—c-Bu | Z-3654 | —C(═O)Et | —C(═O)O—c-Bu |
| Z-3655 | —CH₂CF₂CF₃ | —C(═O)O—c-Pent | Z-3656 | —C(═O)Me | —C(═O)O—c-Pent | Z-3657 | —C(═O)Et | —C(═O)O—c-Pent |
| Z-3658 | —CH₂CF₂CF₃ | —C(═O)O—c-Hex | Z-3659 | —C(═O)Me | —C(═O)O—c-Hex | Z-3660 | —C(═O)Et | —C(═O)O—c-Hex |
| Z-3661 | —CH₂CF₂CF₃ | —C(═O)O—c-Hept | Z-3662 | —C(═O)Me | —C(═O)O—c-Hept | Z-3663 | —C(═O)Et | —C(═O)O—c-Hept |
| Z-3664 | —CH₂CF₂CF₃ | —C(═O)O—c-Oct | Z-3665 | —C(═O)Me | —C(═O)O—c-Oct | Z-3666 | —C(═O)Et | —C(═O)O—c-Oct |
| Z-3667 | —CH₂CF₂CF₃ | —C(═O)OCH═CH₂ | Z-3668 | —C(═O)Me | —C(═O)OCH═CH₂ | Z-3669 | —C(═O)Et | —C(═O)OCH═CH₂ |
| Z-3670 | —CH₂CF₂CF₃ | —C(═O)OCH₂CH═CH₂ | Z-3671 | —C(═O)Me | —C(═O)OCH₂CH═CH₂ | Z-3672 | —C(═O)Et | —C(═O)OCH₂CH═CH₂ |
| Z-3673 | —CH₂CF₂CF₃ | —C(═O)OCH═CHCH₃ | Z-3674 | —C(═O)Me | —C(═O)OCH═CHCH₃ | Z-3675 | —C(═O)Et | —C(═O)OCH═CHCH₃ |
| Z-3676 | —CH₂CF₂CF₃ | —C(═O)OCH₂C(CH₃)═CH₂ | Z-3677 | —C(═O)Me | —C(═O)OCH₂C(CH₃)═CH₂ | Z-3678 | —C(═O)Et | —C(═O)OCH₂C(CH₃)═CH₂ |
| Z-3679 | —CH₂CF₂CF₃ | —C(═O)OCH₂CH₂CH═CH₂ | Z-3680 | —C(═O)Me | —C(═O)OCH₂CH₂CH═CH₂ | Z-3681 | —C(═O)Et | —C(═O)OCH₂CH₂CH═CH₂ |
| Z-3682 | —CH₂CF₂CF₃ | —C(═O)OCH₂CH═CHCH₃ | Z-3683 | —C(═O)Me | —C(═O)OCH₂CH═CHCH₃ | Z-3684 | —C(═O)Et | —C(═O)OCH₂CH═CHCH₃ |
| Z-3685 | —CH₂CF₂CF₃ | —C(═O)OCH═CHCH₂CH₃ | Z-3686 | —C(═O)Me | —C(═O)OCH═CHCH₂CH₃ | Z-3687 | —C(═O)Et | —C(═O)OCH═CHCH₂CH₃ |
| Z-3688 | —CH₂CF₂CF₃ | —C(═O)OCH₂CH═C(CH₃)₂ | Z-3689 | —C(═O)Me | —C(═O)OCH₂CH═C(CH₃)₂ | Z-3690 | —C(═O)Et | —C(═O)OCH₂CH═C(CH₃)₂ |
| Z-3691 | —CH₂CF₂CF₃ | —C(═O)OCH2CH₂CH═C(CH₃)₂ | Z-3692 | —C(═O)Me | —C(═O)OCH2CH₂CH═C(CH₃)₂ | Z-3693 | —C(═O)Et | —C(═O)OCH2CH₂CH═C(CH₃)₂ |
| Z-3694 | —CH₂CF₂CF₃ | —C(═O)OCH═CFH | Z-3695 | —C(═O)Me | —C(═O)OCH═CFH | Z-3696 | —C(═O)Et | —C(═O)OCH═CFH |
| Z-3697 | —CH₂CF₂CF₃ | —C(═O)OCH═CF₂ | Z-3698 | —C(═O)Me | —C(═O)OCH═CF₂ | Z-3699 | —C(═O)Et | —C(═O)OCH═CF₂ |
| Z-3700 | —CH₂CF₂CF₃ | —C(═O)OCH═CCl₂ | Z-3701 | —C(═O)Me | —C(═O)OCH═CCl₂ | Z-3702 | —C(═O)Et | —C(═O)OCH═CCl₂ |
| Z-3703 | —CH₂CF₂CF₃ | —C(═O)OCH₂CH═CFH | Z-3704 | —C(═O)Me | —C(═O)OCH₂CH═CFH | Z-3705 | —C(═O)Et | —C(═O)OCH₂CH═CFH |
| Z-3706 | —CH₂CF₂CF₃ | —C(═O)OCH₂CH═CF₂ | Z-3707 | —C(═O)Me | —C(═O)OCH₂CH═CF₂ | Z-3708 | —C(═O)Et | —C(═O)OCH₂CH═CF₂ |
| Z-3709 | —CH₂CF₂CF₃ | —C(═O)OCH₂CH═CCl₂ | Z-3710 | —C(═O)Me | —C(═O)OCH₂CH═CCl₂ | Z-3711 | —C(═O)Et | —C(═O)OCH₂CH═CCl₂ |
| Z-3712 | —CH₂CF₂CF₃ | —C(═O)OCH₂CH₂CH═CF₂ | Z-3713 | —C(═O)Me | —C(═O)OCH₂CH₂CH═CF₂ | Z-3714 | —C(═O)Et | —C(═O)OCH₂CH₂CH═CF₂ |
| Z-3715 | —CH₂CF₂CF₃ | —C(═O)OCH₂CH₂CH₂CH═CF₂ | Z-3716 | —C(═O)Me | —C(═O)OCH₂CH₂CH₂CH═CF₂ | Z-3717 | —C(═O)Et | —C(═O)OCH₂CH₂CH₂CH═CF₂ |
| Z-3718 | —CH₂CF₂CF₃ | —C(═O)OCH₂CH₂CH₂CH₂CH═CF₂ | Z-3719 | —C(═O)Me | —C(═O)OCH₂CH₂CH₂CH₂CH═CF₂ | Z-3720 | —C(═O)Et | —C(═O)OCH₂CH₂CH₂CH₂CH═CF₂ |
| Z-3721 | —CH₂CF₂CF₃ | —C(═Q)OCH₂C≡CH | Z-3722 | —C(═O)Me | —C(═Q)OCH₂C≡CH | Z-3723 | —C(═O)Et | —C(═Q)OCH₂C≡CH |
| Z-3724 | —CH₂CF₂CF₃ | —C(═O)OCH₂C≡CCH₃ | Z-3725 | —C(═O)Me | —C(═O)OCH₂C≡CCH₃ | Z-3726 | —C(═O)Et | —C(═O)OCH₂C≡CCH₃ |
| Z-3727 | —CH₂CF₂CF₃ | —C(═O)OCH₂CH₂C≡CH | Z-3728 | —C(═O)Me | —C(═O)OCH₂CH₂C≡CH | Z-3729 | —C(═O)Et | —C(═O)OCH₂CH₂C≡CH |
| Z-3730 | —CH₂CF₂CF₃ | —C(═O)OCH₂C≡CCH₂CH₃ | Z-3731 | —C(═O)Me | —C(═O)OCH₂C≡CCH₂CH₃ | Z-3732 | —C(═O)Et | —C(═O)OCH₂C≡CCH₂CH₃ |
| Z-3733 | —CH₂CF₂CF₃ | —C(═O)OC(CH₃)₂C≡CH | Z-3734 | —C(═O)Me | —C(═O)OC(CH₃)₂C≡CH | Z-3735 | —C(═O)Et | —C(═O)OC(CH₃)₂C≡CH |
| Z-3736 | —CH₂CF₂CF₃ | —C(═O)OCH₂C≡CHCF₂H | Z-3737 | —C(═O)Me | —C(═O)OCH₂C≡CHCF₂H | Z-3738 | —C(═O)Et | —C(═O)OCH₂C≡CHCF₂H |
| Z-3739 | —CH₂CF₂CF₃ | —C(═O)OCH₂C≡CCF₃ | Z-3740 | —C(═O)Me | —C(═O)OCH₂C≡CCF₃ | Z-3741 | —C(═O)Et | —C(═O)OCH₂C≡CCF₃ |
| Z-3742 | —CH₂CF₂CF₃ | —C(═O)Ph | Z-3743 | —C(═O)Me | —C(═O)Ph | Z-3744 | —C(═O)Et | —C(═O)Ph |
| Z-3745 | —CH₂CF₂CF₃ | —C(═O)(2-Py) | Z-3746 | —C(═O)Me | —C(═O)(2-Py) | Z-3747 | —C(═O)Et | —C(═O)(2-Py) |
| Z-3748 | —CH₂CF₂CF₃ | —C(═O)(3-Py) | Z-3749 | —C(═O)Me | —C(═O)(3-Py) | Z-3750 | —C(═O)Et | —C(═O)(3-Py) |
| Z-3751 | —CH₂CF₂CF₃ | —C(═O)(4-Py) | Z-3752 | —C(═O)Me | —C(═O)(4-Py) | Z-3753 | —C(═O)Et | —C(═O)(4-Py) |
| Z-3754 | —CH₂CF₂CF₃ | —C(═O)CF₂Me | Z-3755 | —C(═O)Me | —C(═O)CF₂Me | Z-3756 | —C(═O)Et | —C(═O)CF₂Me |
| Z-3757 | —CH₂CF₂CF₃ | —C(═O)NMe2 | Z-3758 | —C(═O)Me | —C(═O)NMe2 | Z-3759 | —C(═O)Et | —C(═O)NMe2 |
| Z-3760 | —CH₂CF₂CF₃ | —C(═O)—(1-CF₃—c-Pr) | Z-3761 | —C(═O)Me | —C(═O)—(1-CF₃—c-Pr) | Z-3762 | —C(═O)Et | —C(═O)—(1-CF₃—c-Pr) |
| Z-3763 | —CH₂CF₂CF₃ | —C(═O)—(1-F—c-Pr) | Z-3764 | —C(═O)Me | —C(═O)—(1-F—c-Pr) | Z-3765 | —C(═O)Et | —C(═O)—(1-F—c-Pr) |
| Z-3766 | —CH₂CF₂CF₃ | —SO₂Me | Z-3767 | —C(═O)Me | —SO₂Me | Z-3768 | —C(═O)Et | —SO₂Me |
| Z-3769 | —CH₂CF₂CF₃ | —C(═O)CF═CH₂ | Z-3770 | —C(═O)Me | —C(═O)CF═CH₂ | Z-3771 | —C(═O)Et | —C(═O)CF═CH₂ |
| Z-3772 | —CH₂CF₂CF₃ | —C(═O)—(4-Cl—Ph) | Z-3773 | —C(═O)Me | —C(═O)—(4-Cl—Ph) | Z-3774 | —C(═O)Et | —C(═O)—(4-Cl—Ph) |
| Z-3775 | —CH₂CF₂CF₃ | —C(═O)—(3-Cl—Ph) | Z-3776 | —C(═O)Me | —C(═O)—(3-Cl—Ph) | Z-3777 | —C(═O)Et | —C(═O)—(3-Cl—Ph) |
| Z-3778 | —CH₂CF₂CF₃ | —C(═O)—(3-CF₃—Ph) | Z-3779 | —C(═O)Me | —C(═O)—(3-CF₃—Ph) | Z-3780 | —C(═O)Et | —C(═O)—(3-CF₃—Ph) |
| Z-3781 | —CH₂CF₂CF₃ | —C(═O)—(2-Cl—Ph) | Z-3782 | —C(═O)Me | —C(═O)—(2-Cl—Ph) | Z-3783 | —C(═O)Et | —C(═O)—(2-Cl—Ph) |
| Z-3784 | —CH₂CF₂CF₃ | —C(═O)—(2-CF₃—Ph) | Z-3785 | —C(═O)Me | —C(═O)—(2-CF₃—Ph) | Z-3786 | —C(═O)Et | —C(═O)—(2-CF₃—Ph) |
| Z-3787 | —CH₂CF₂CF₃ | —C(═O)—(4-CF₃—Ph) | Z-3788 | —C(═O)Me | —C(═O)—(4-CF₃—Ph) | Z-3789 | —C(═O)Et | —C(═O)—(4-CF₃—Ph) |
| Z-3790 | —CH₂CF₂CF₃ | —C(═O)—(3-F—Ph) | Z-3791 | —C(═O)Me | —C(═O)—(3-F—Ph) | Z-3792 | —C(═O)Et | —C(═O)—(3-F—Ph) |
| Z-3793 | —CH₂CF₂CF₃ | —C(═O)—(4-F—Ph) | Z-3794 | —C(═O)Me | —C(═O)—(4-F—Ph) | Z-3795 | —C(═O)Et | —C(═O)—(4-F—Ph) |
| Z-3796 | —CH₂CF₂CF₃ | —C(═O)—(2-F—Ph) | Z-3797 | —C(═O)Me | —C(═O)—(2-F—Ph) | Z-3798 | —C(═O)Et | —C(═O)—(2-F—Ph) |
| Z-3799 | —CH₂CF₂CF₃ | —C(═O)—(4-OCF₃—Ph) | Z-3800 | —C(═O)Me | —C(═O)—(4-OCF₃—Ph) | Z-3801 | —C(═O)Et | —C(═O)—(4-OCF₃—Ph) |
| Z-3802 | —CH₂CF₂CF₃ | —C(═O)—(6-Cl—3-Py) | Z-3803 | —C(═O)Me | —C(═O)—(6-Cl—3-Py) | Z-3804 | —C(═O)Et | —C(═O)—(6-Cl—3-Py) |
| Z-3805 | —CH₂CF₂CF₃ | —C(═O)—(6-CF3—2-Py) | Z-3806 | —C(═O)Me | —C(═O)—(6-CF3—2-Py) | Z-3807 | —C(═O)Et | —C(═O)—(6-CF3—2-Py) |
| Z-3808 | —CH₂CF₂CF₃ | —C(═O)—(1-CN—c-Pr) | Z-3809 | —C(═O)Me | —C(═O)—(1-CN—c-Pr) | Z-3810 | —C(═O)Et | —C(═O)—(1-CN—c-Pr) |
| Z-3811 | —CH₂CF₂CF₃ | —C(═O)—(3-Cl—2-Py) | Z-3812 | —C(═O)Me | —C(═O)—(3-Cl—2-Py) | Z-3813 | —C(═O)Et | —C(═O)—(3-Cl—2-Py) |
| Z-3814 | —CH₂CF₂CF₃ | —C(═O)—(2-Me—Ph) | Z-3815 | —C(═O)Me | —C(═O)—(2-Me—Ph) | Z-3816 | —C(═O)Et | —C(═O)—(2-Me—Ph) |
| Z-3817 | —CH₂CF₂CF₃ | —C(═O)—(3-Me—Ph) | Z-3818 | —C(═O)Me | —C(═O)—(3-Me—Ph) | Z-3819 | —C(═O)Et | —C(═O)—(3-Me—Ph) |
| Z-3820 | —CH₂CF₂CF₃ | —C(═O)—(4-Me—Ph) | Z-3821 | —C(═O)Me | —C(═O)—(4-Me—Ph) | Z-3822 | —C(═O)Et | —C(═O)—(4-Me—Ph) |
| Z-3823 | —CH₂CF₂CF₃ | —C(═O)—(2-MeO—Ph) | Z-3824 | —C(═O)Me | —C(═O)—(2-MeO—Ph) | Z-3825 | —C(═O)Et | —C(═O)—(2-MeO—Ph) |
| Z-3826 | —CH₂CF₂CF₃ | —C(═O)—(3-MeO—Ph) | Z-3827 | —C(═O)Me | —C(═O)—(3-MeO—Ph) | Z-3828 | —C(═O)Et | —C(═O)—(3-MeO—Ph) |
| Z-3829 | —CH₂CF₂CF₃ | —C(═O)—(4-MeO—Ph) | Z-3830 | —C(═O)Me | —C(═O)—(4-MeO—Ph) | Z-3831 | —C(═O)Et | —C(═O)—(4-MeO—Ph) |
| Z-3832 | —CH₂CF₂CF₃ | —C(═O)—(4-Cl—2-Py) | Z-3833 | —C(═O)Me | —C(═O)—(4-Cl—2-Py) | Z-3834 | —C(═O)Et | —C(═O)—(4-Cl—2-Py) |
| Z-3835 | —CH₂CF₂CF₃ | —C(═O)—(5-Cl—2-Py) | Z-3836 | —C(═O)Me | —C(═O)—(5-Cl—2-Py) | Z-3837 | —C(═O)Et | —C(═O)—(5-Cl—2-Py) |
| Z-3838 | —CH₂CF₂CF₃ | —C(═O)—(6-Cl—2-Py) | Z-3839 | —C(═O)Me | —C(═O)—(6-Cl—2-Py) | Z-3840 | —C(═O)Et | —C(═O)—(6-Cl—2-Py) |
| Z-3841 | —CH₂CF₂CF₃ | —C(═O)—(2-Cl—3-Py) | Z-3842 | —C(═O)Me | —C(═O)—(2-Cl—3-Py) | Z-3843 | —C(═O)Et | —C(═O)—(2-Cl—3-Py) |
| Z-3844 | —CH₂CF₂CF₃ | —C(═O)—(2-Cl—4-Py) | Z-3845 | —C(═O)Me | —C(═O)—(2-Cl—4-Py) | Z-3846 | —C(═O)Et | —C(═O)—(2-Cl—4-Py) |
| Z-3847 | —CH₂CF₂CF₃ | —C(═O)—(3-Cl—4-Py) | Z-3848 | —C(═O)Me | —C(═O)—(3-Cl—4-Py) | Z-3849 | —C(═O)Et | —C(═O)—(3-Cl—4-Py) |
| Z-3850 | —CH₂CF₂CF₃ | —C(═O)—(3,4-di-Me—Ph) | Z-3851 | —C(═O)Me | —C(═O)—(3,4-di-Me—Ph) | Z-3852 | —C(═O)Et | —C(═O)—(3,4-di-Me—Ph) |
| Z-3853 | —CH₂CF₂CF₃ | —C(═O)—(3,5-di-Me—Ph) | Z-3854 | —C(═O)Me | —C(═O)—(3,5-di-Me—Ph) | Z-3855 | —C(═O)Et | —C(═O)—(3,5-di-Me—Ph) |
| Z-3856 | —CH₂CF₂CF₃ | —C(═O)—(4-Cl—3-Py) | Z-3857 | —C(═O)Me | —C(═O)—(4-Cl—3-Py) | Z-3858 | —C(═O)Et | —C(═O)—(4-Cl—3-Py) |
| Z-3859 | —CH₂CF₂CF₃ | —C(═O)—(5-Cl—3-Py) | Z-3860 | —C(═O)Me | —C(═O)—(5-Cl—3-Py) | Z-3861 | —C(═O)Et | —C(═O)—(5-Cl—3-Py) |
| Z-3862 | —CH₂CF₂CF₃ | —C(═O)—(4-Pyrimidine) | Z-3863 | —C(═O)Me | —C(═O)—(4-Pyrimidine) | Z-3864 | —C(═O)Et | —C(═O)—(4-Pyrimidine) |
| Z-3865 | —CH₂CF₂CF₃ | —C(═O)—(2-Cl—4-Pyrimidine) | Z-3866 | —C(═O)Me | —C(═O)—(2-Cl—4-Pyrimidine) | Z-3867 | —C(═O)Et | —C(═O)—(2-Cl—4-Pyrimidine) |
| Z-3868 | —CH₂CF₂CF₃ | —C(═O)—(4-Et—Ph) | Z-3869 | —C(═O)Me | —C(═O)—(4-Et—Ph) | Z-3870 | —C(═O)Et | —C(═O)—(4-Et—Ph) |
| Z-3871 | —CH₂CF₂CF₃ | —C(═O)—(2-Me—4-Pyrimidine) | Z-3872 | —C(═O)Me | —C(═O)—(2-Me—4-Pyrimidine) | Z-3873 | —C(═O)Et | —C(═O)—(2-Me—4-Pyrimidine) |
| Z-3874 | —CH₂CF₂CF₃ | —C(═O)—(6-Me—4-Pyrimidine) | Z-3875 | —C(═O)Me | —C(═O)—(6-Me—4-Pyrimidine) | Z-3876 | —C(═O)Et | —C(═O)—(6-Me—4-Pyrimidine) |
| Z-3877 | —CH₂CF₂CF₃ | —C(═O)—(6-Me—2-Py) | Z-3878 | —C(═O)Me | —C(═O)—(6-Me—2-Py) | Z-3879 | —C(═O)Et | —C(═O)—(6-Me—2-Py) |
| Z-3880 | —CH₂CF₂CF₃ | —C(═O)—(2-CF₃—4-Pyrimidine) | Z-3881 | —C(═O)Me | —C(═O)—(2-CF₃—4-Pyrimidine) | Z-3882 | —C(═O)Et | —C(═O)—(2-CF₃—4-Pyrimidine) |
| Z-3883 | —CH₂CF₂CF₃ | —C(═O)—(3-Pyridazine) | Z-3884 | —C(═O)Me | —C(═O)—(3-Pyridazine) | Z-3885 | —C(═O)Et | —C(═O)—(3-Pyridazine) |
| Z-3886 | —CH₂CF₂CF₃ | —C(═O)—(1-Me—c-Pr) | Z-3887 | —C(═O)Me | —C(═O)—(1-Me—c-Pr) | Z-3888 | —C(═O)Et | —C(═O)—(1-Me—c-Pr) |
| Z-3889 | —CH₂CF₂CF₃ | —C(═O)—(1-CF₃—c-Bu) | Z-3890 | —C(═O)Me | —C(═O)—(1-CF₃—c-Bu) | Z-3891 | —C(═O)Et | —C(═O)—(1-CF₃—c-Bu) |
| Z-3892 | —CH₂CF₂CF₃ | —C(═O)—(2-Pyrimidine) | Z-3893 | —C(═O)Me | —C(═O)—(2-Pyrimidine) | Z-3894 | —C(═O)Et | —C(═O)—(2-Pyrimidine) |
| Z-3895 | —CH₂CF₂CF₃ | —C(═O)—(2-Pyrazine) | Z-3896 | —C(═O)Me | —C(═O)—(2-Pyrazine) | Z-3897 | —C(═O)Et | —C(═O)—(2-Pyrazine) |
| Z-3898 | —CH₂CF₂CF₃ | —C(═O)CH═CHOEt | Z-3899 | —C(═O)Me | —C(═O)CH═CHOEt | Z-3900 | —C(═O)Et | —C(═O)CH═CHOEt |
| Z-3901 | —CH₂CF₂CF₃ | —C(═O)CH₂CHCF₃CF₃ | Z-3902 | —C(═O)Me | —C(═O)CH₂CHCF₃CF₃ | Z-3903 | —C(═O)Et | —C(═O)CH₂CHCF₃CF₃ |
| Z-3904 | —CH₂CF₂CF₃ | —C(═O)CH₂—(c-Pr) | Z-3905 | —C(═O)Me | —C(═O)CH₂—(c-Pr) | Z-3906 | —C(═O)Et | —C(═O)CH₂—(c-Pr) |
| Z-3907 | —CH₂CF₂CF₃ | —C(═O)—(2,2,3,3-tetra-Me—c-Pr) | Z-3908 | —C(═O)Me | —C(═O)—(2,2,3,3-tetra-Me—c-Pr) | Z-3909 | —C(═O)Et | —C(═O)—(2,2,3,3-tetra-Me—c-Pr) |
| Z-3910 | —CH₂CF₂CF₃ | —C(═O)CMe₂CF₃ | Z-3911 | —C(═O)Me | —C(═O)CMe₂CF₃ | Z-3912 | —C(═O)Et | —C(═O)CMe₂CF₃ |
| Z-3913 | —C(═O)—c-Pr | —H | Z-3914 | —C(═O)OMe | —H | Z-3915 | —C(═O)Et | —H |
| Z-3916 | —C(═O)—c-Pr | —Me | Z-3917 | —C(═O)OMe | —Me | Z-3918 | —C(═O)Et | —Me |
| Z-3919 | —C(═O)—c-Pr | —Et | Z-3920 | —C(═O)OMe | —Et | Z-3921 | —C(═O)Et | —Et |
| Z-3922 | —C(═O)—c-Pr | —Pr | Z-3923 | —C(═O)OMe | —Pr | Z-3924 | —C(═O)Et | —Pr |
| Z-3925 | —C(═O)—c-Pr | —i-Pr | Z-3926 | —C(═O)OMe | —i-Pr | Z-3927 | —C(═O)Et | —i-Pr |
| Z-3928 | —C(═O)—c-Pr | —Bu | Z-3929 | —C(═O)OMe | —Bu | 2-3930 | —C(═O)Et | —Bu |
| Z-3931 | —C(═O)—c-Pr | —sec-Bu | Z-3932 | —C(═O)OMe | —sec-Bu | Z-3933 | —C(═O)Et | —sec-Bu |
| Z-3934 | —C(═O)—c-Pr | —I-Bu | Z-3935 | —C(═O)OMe | —I-Bu | Z-3936 | —C(═O)Et | —I-Bu |
| Z-3937 | —C(═O)—c-Pr | —t-Bu | Z-3938 | —C(═O)OMe | —t-Bu | Z-3939 | —C(═O)Et | —t-Bu |
| Z-3940 | —C(═O)—c-Pr | —Pent | Z-3941 | —C(═O)OMe | —Pent | Z-3942 | —C(═O)Et | —Pent |
| Z-3943 | —C(═O)—c-Pr | —Hex | Z-3944 | —C(═O)OMe | —Hex | Z-3945 | —C(═O)Et | —Hex |
| Z-3946 | —C(═O)—c-Pr | —CH(CH₃)CH₂CH₂CH₃ | Z-3947 | —C(═O)OMe | —CH(CH₃)CH₂CH₂CH₃ | Z-3948 | —C(═O)Et | —CH(CH₃)CH₂CH₂CH₃ |
| Z-3949 | —C(═O)—c-Pr | —CH(CH₃)CH(CH₃)₂ | Z-3950 | —C(═O)OMe | —CH(CH₃)CH(CH₃)₂ | Z-3951 | —C(═O)Et | —CH(CH₃)CH(CH₃)₂ |
| Z-3952 | —C(═O)—c-Pr | —C(CH₃)₂CH₂CH₃ | Z-3953 | —C(═O)OMe | —C(CH₃)₂CH₂CH₃ | Z-3954 | —C(═O)Et | —C(CH₃)₂CH₂CH₃ |
| Z-3955 | —C(═O)—c-Pr | —CH(CH₂CH₃)₂ | Z-3956 | —C(═O)OMe | —CH(CH₂CH₃)₂ | Z-3957 | —C(═O)Et | —CH(CH₂CH₃)₂ |
| Z-3958 | —C(═O)—c-Pr | —CH₂CH₂CH(CH₃)₂ | Z-3959 | —C(═O)OMe | —CH₂CH₂CH(CH₃)₂ | Z-3960 | —C(═O)Et | —CH₂CH₂CH(CH₃)₂ |
| Z-3961 | —C(═O)—c-Pr | —CH₂CH₂CH₂CH(CH₃)₂ | Z-3962 | —C(═O)OMe | —CH₂CH₂CH₂CH(CH₃)₂ | Z-3963 | —C(═O)Et | —CH₂CH₂CH₂CH(CH₃)₂ |
| Z-3964 | —C(═O)—c-Pr | —CH₂CH₂CH(CH₃)CH₂CH₃ | Z-3965 | —C(═O)OMe | —CH₂CH₂CH(CH₃)CH₂CH₃ | Z-3966 | —C(═O)Et | —CH₂CH₂CH(CH₃)CH₂CH₃ |
| Z-3967 | —C(═O)—c-Pr | —CH₂CH(CH₃)CH₂CH₂CH₃ | Z-3968 | —C(═O)OMe | —CH₂CH(CH₃)CH₂CH₂CH₃ | Z-3969 | —C(═O)Et | —CH₂CH(CH₃)CH₂CH₂CH₃ |
| Z-3970 | —C(═O)—c-Pr | —CH(CH₃)CH₂CH₂CH₂CH₃ | Z-3971 | —C(═O)OMe | —CH(CH₃)CH₂CH₂CH₂CH₃ | Z-3972 | —C(═O)Et | —CH(CH₃)CH₂CH₂CH₂CH₃ |
| Z-3973 | —C(═O)—c-Pr | —CH(CH3)CH₂CH(CH₃)₂ | Z-3974 | —C(═O)OMe | —CH(CH3)CH₂CH(CH₃)₂ | Z-3975 | —C(═O)Et | —CH(CH3)CH₂CH(CH₃)₂ |
| Z-3976 | —C(═O)—c-Pr | —CH(CH₃)CH(CH₃)CH₂CH₃ | Z-3977 | —C(═O)OMe | —CH(CH₃)CH(CH₃)CH₂CH₃ | Z-3978 | —C(═O)Et | —CH(CH₃)CH(CH₃)CH₂CH₃ |
| Z-3979 | —C(═O)—c-Pr | —C(CH₃)₂CH₂CH₂CH₃ | Z-3980 | —C(═O)OMe | —C(CH₃)₂CH₂CH₂CH₃ | Z-3981 | —C(═O)Et | —C(CH₃)₂CH₂CH₂CH₃ |
| Z-3982 | —C(═O)—c-Pr | —CH(CH₃)C(CH₃)₃ | Z-3983 | —C(═O)OMe | —CH(CH₃)C(CH₃)₃ | Z-3984 | —C(═O)Et | —CH(CH₃)C(CH₃)₃ |
| Z-3985 | —C(═O)—c-Pr | —C(CH₃)₂CH(CH₃)₂ | Z-3986 | —C(═O)OMe | —C(CH₃)₂CH(CH₃)₂ | Z-3987 | —C(═O)Et | —C(CH₃)₂CH(CH₃)₂ |
| Z-3988 | —C(═O)—c-Pr | —CH₂CH₂C(CH₃)₃ | Z-3989 | —C(═O)OMe | —CH₂CH₂C(CH₃)₃ | Z-3990 | —C(═O)Et | —CH₂CH₂C(CH₃)₃ |
| Z-3991 | —C(═O)—c-Pr | —CH₂CH(CH₃)CH(CH₃)₂ | Z-3992 | —C(═O)OMe | —CH₂CH(CH₃)CH(CH₃)₂ | Z-3993 | —C(═O)Et | —CH₂CH(CH₃)CH(CH₃)₂ |
| Z-3994 | —C(═O)—c-Pr | —CH₂C(CH₃)₂CH₂CH₃ | Z-3995 | —C(═O)OMe | —CH₂C(CH₃)₂CH₂CH₃ | Z-3996 | —C(═O)Et | —CH₂C(CH₃)₂CH₂CH₃ |
| Z-3997 | —C(═O)—c-Pr | —CFH₂ | Z-3998 | —C(═O)OMe | —CFH₂ | Z-3999 | —C(═O)Et | —CFH₂ |
| Z-4000 | —C(═O)—c-Pr | —CF₂H | Z-4001 | —C(═O)OMe | —CF₂H | Z-4002 | —C(═O)Et | —CF₂H |
| Z-4003 | —C(═O)—c-Pr | —CF₃ | Z-4004 | —C(═O)OMe | —CF₃ | Z-4005 | —C(═O)Et | —CF₃ |
| Z-4006 | —C(═O)—c-Pr | —CH₂Cl | Z-4007 | —C(═O)OMe | —CH₂Cl | Z-4008 | —C(═O)Et | —CH₂Cl |
| Z-4009 | —C(═O)—c-Pr | —CHCl₂ | Z-4010 | —C(═O)OMe | —CHCl₂ | Z-4011 | —C(═O)Et | —CHCl₂ |
| Z-4012 | —C(═O)—c-Pr | —CCl₃ | Z-4013 | —C(═O)OMe | —CCl₃ | Z-4014 | —C(═O)Et | —CCl₃ |
| Z-4015 | —C(═O)—c-Pr | —CF₂Cl | Z-4016 | —C(═O)OMe | —CF₂Cl | Z-4017 | —C(═O)Et | —CF₂Cl |
| Z-4018 | —C(═O)—c-Pr | —CCl₂F | Z-4019 | —C(═O)OMe | —CCl₂F | Z-4020 | —C(═O)Et | —CCl₂F |
| Z-4021 | —C(═O)—c-Pr | —CH₂Br | Z-4022 | —C(═O)OMe | —CH₂Br | Z-4023 | —C(═O)Et | —CH₂Br |
| Z-4024 | —C(═O)—c-Pr | —CHBr₂ | Z-4025 | —C(═O)OMe | —CHBr₂ | Z-4026 | —C(═O)Et | —CHBr₂ |
| Z-4027 | —C(═O)—c-Pr | —CBr₃ | Z-4028 | —C(═O)OMe | —CBr₃ | Z-4029 | —C(═O)Et | —CBr₃ |
| Z-4030 | —C(═O)—c-Pr | —CH₂l | Z-4031 | —C(═O)OMe | —CH₂l | Z-4032 | —C(═O)Et | —CH₂l |
| Z-4033 | —C(═O)—c-Pr | —CHl₂ | Z-4034 | —C(═O)OMe | —CHl₂ | Z-4035 | —C(═O)Et | —CHl₂ |
| Z-4036 | —C(═O)—c-Pr | —CH₂CF₂H | Z-4037 | —C(═O)OMe | —CH₂CF₂H | Z-4038 | —C(═O)Et | —CH₂CF₂H |
| Z-4039 | —C(═O)—c-Pr | —CH₂CF₃ | Z-4040 | —C(═O)OMe | —CH₂CF₃ | Z-4041 | —C(═O)Et | —CH₂CF₃ |
| Z-4042 | —C(═O)—c-Pr | —CF₂CH₃ | Z-4043 | —C(═O)OMe | —CF₂CH₃ | Z-4044 | —C(═O)Et | —CF₂CH₃ |
| Z-4045 | —C(═O)—c-Pr | —CH₂CH₂CF₂H | Z-4046 | —C(═O)OMe | —CH₂CH₂CF₂H | Z-4047 | —C(═O)Et | —CH₂CH₂CF₂H |
| Z-4048 | —C(═O)—c-Pr | —CH₂CH₂CF₃ | Z-4049 | —C(═O)OMe | —CH₂CH₂CF₃ | Z-4050 | —C(═O)Et | —CH₂CH₂CF₃ |
| Z-4051 | —C(═O)—c-Pr | —CH₂CH₂CH₂CF₂H | Z-4052 | —C(═O)OMe | —CH₂CH₂CH₂CF₂H | Z-4053 | —C(═O)Et | —CH₂CH₂CH₂CF₂H |
| Z-4054 | —C(═O)—c-Pr | —CH₂CH₂CH₂CF₃ | Z-4055 | —C(═O)OMe | —CH₂CH₂CH₂CF₃ | Z-4056 | —C(═O)Et | —CH₂CH₂CH₂CF₃ |
| Z-4057 | —C(═O)—c-Pr | —CF₂CF₂H | Z-4058 | —C(═O)OMe | —CF₂CF₂H | Z-4059 | —C(═O)Et | —CF₂CF₂H |
| Z-4060 | —C(═O)—c-Pr | —CF₂CF₂Cl | Z-4061 | —C(═O)OMe | —CF₂CF₂Cl | Z-4062 | —C(═O)Et | —CF₂CF₂Cl |
| Z-4063 | —C(═O)—c-Pr | —CF₂CF₃ | Z-4064 | —C(═O)OMe | —CF₂CF₃ | Z-4065 | —C(═O)Et | —CF₂CF₃ |
| Z-4066 | —C(═O)—c-Pr | —CFHCF₃ | Z-4067 | —C(═O)OMe | —CFHCF₃ | Z-4068 | —C(═O)Et | —CFHCF₃ |
| Z-4069 | —C(═O)—c-Pr | —CH₂CF₂CF₂H | Z-4070 | —C(═O)OMe | —CH₂CF₂CF₂H | Z-4071 | —C(═O)Et | —CH₂CF₂CF₂H |
| Z-4072 | —C(═O)—c-Pr | —CH₂CF₂CF₃ | Z-4073 | —C(═O)OMe | —CH₂CF₂CF₃ | Z-4074 | —C(═O)Et | —CH₂CF₂CF₃ |
| Z-4075 | —C(═O)—c-Pr | —CF₂CF₂CF₃ | Z-4076 | —C(═O)OMe | —CF₂CF₂CF₃ | Z-4077 | —C(═O)Et | —CF₂CF₂CF₃ |
| Z-4078 | —C(═O)—c-Pr | —CH₂CF₂CF₂CF₃ | Z-4079 | —C(═O)OMe | —CH₂CF₂CF₂CF₃ | Z-4080 | —C(═O)Et | —CH₂CF₂CF₂CF₃ |
| Z-4081 | —C(═O)—c-Pr | —CF₂CF₂CF₂CF₃ | Z-4082 | —C(═O)OMe | —CF₂CF₂CF₂CF₃ | Z-4083 | —C(═O)Et | —CF₂CF₂CF₂CF₃ |
| Z-4084 | —C(═O)—c-Pr | —CH₂CF₂CF₂CF₂CF₃ | Z-4085 | —C(═O)OMe | —CH₂CF₂CF₂CF₂CF₃ | Z-4086 | —C(═O)Et | —CH₂CF₂CF₂CF₂CF₃ |
| Z-4087 | —C(═O)—c-Pr | c-Pr | Z-4088 | —C(═O)OMe | c-Pr | Z-4089 | —C(═O)Et | c-Pr |
| Z-4090 | —C(═O)—c-Pr | c-Bu | Z-4091 | —C(═O)OMe | c-Bu | Z-4092 | —C(═O)Et | c-Bu |
| Z-4093 | —C(═O)—c-Pr | c-Pent | Z-4094 | —C(═O)OMe | c-Pent | Z-4095 | —C(═O)Et | c-Pent |
| Z-4096 | —C(═O)—c-Pr | c-Hex | Z-4097 | —C(═O)OMe | c-Hex | Z-4098 | —C(═O)Et | c-Hex |
| Z-4099 | —C(═O)—c-Pr | c-Hept | Z-4100 | —C(═O)OMe | c-Hept | Z-4101 | —C(═O)Et | c-Hept |
| Z-4102 | —C(═O)—c-Pr | c-Oct | Z-4103 | —C(═O)OMe | c-Oct | Z-4104 | —C(═O)Et | c-Oct |
| Z-4105 | —C(═O)—c-Pr | —CH═CH₂ | Z-4106 | —C(═O)OMe | —CH═CH₂ | Z-4107 | —C(═O)Et | —CH═CH₂ |
| Z-4108 | —C(═O)—c-Pr | —CH₂CH═CH₂ | Z-4109 | —C(═O)OMe | —CH₂CH═CH₂ | Z-4110 | —C(═O)Et | —CH₂CH═CH₂ |
| Z-4111 | —C(═O)—c-Pr | —CH═CHCH₃ | Z-4112 | —C(═O)OMe | —CH═CHCH₃ | Z-4113 | —C(═O)Et | —CH═CHCH₃ |
| Z-4114 | —C(═O)—c-Pr | —CH₂C(CH₃)═CH₂ | Z-4115 | —C(═O)OMe | —CH₂C(CH₃)═CH₂ | Z-4116 | —C(═O)Et | —CH₂C(CH₃)═CH₂ |
| Z-4117 | —C(═O)—c-Pr | —CH₂CH₂CH═CH₂ | Z-4118 | —C(═O)OMe | —CH₂CH₂CH═CH₂ | Z-4119 | —C(═O)Et | —CH₂CH₂CH═CH₂ |
| Z-4120 | —C(═O)—c-Pr | —CH₂CH═CHCH₃ | Z-4121 | —C(═O)OMe | —CH₂CH═CHCH₃ | Z-4122 | —C(═O)Et | —CH₂CH═CHCH₃ |
| Z-4123 | —C(═O)—c-Pr | —CH═CHCH₂CH₃ | Z-4124 | —C(═O)OMe | —CH═CHCH₂CH₃ | Z-4125 | —C(═O)Et | —CH═CHCH₂CH₃ |
| Z-4126 | —C(═O)—c-Pr | —CH₂CH═C(CH₃)₂ | Z-4127 | —C(═O)OMe | —CH₂CH═C(CH₃)₂ | Z-4128 | —C(═O)Et | —CH₂CH═C(CH₃)₂ |
| Z-4129 | —C(═O)—c-Pr | —CH₂CH₂CH═C(CH₃)₂ | Z-4130 | —C(═O)OMe | —CH₂CH₂CH═C(CH₃)₂ | Z-4131 | —C(═O)Et | —CH₂CH₂CH═C(CH₃)₂ |
| Z-4132 | —C(═O)—c-Pr | —CH═CFH | Z-4133 | —C(═O)OMe | —CH═CFH | Z-4134 | —C(═O)Et | —CH═CFH |
| Z-4135 | —C(═O)—c-Pr | —CH═CF₂ | Z-4136 | —C(═O)OMe | —CH═CF₂ | Z-4137 | —C(═O)Et | —CH═CF₂ |
| Z-4138 | —C(═O)—c-Pr | —CH═CCl₂ | Z-4139 | —C(═O)OMe | —CH═CCl₂ | Z-4140 | —C(═O)Et | —CH═CCl₂ |
| Z-4141 | —C(═O)—c-Pr | —CH₂CH═CFH | Z-4142 | —C(═O)OMe | —CH₂CH═CFH | Z-4143 | —C(═O)Et | —CH₂CH═CFH |
| Z-4144 | —C(═O)—c-Pr | —CH₂CH═CF₂ | Z-4145 | —C(═O)OMe | —CH₂CH═CF₂ | Z-4146 | —C(═O)Et | —CH₂CH═CF₂ |
| Z-4147 | —C(═O)—c-Pr | —CH₂CH═CCl₂ | Z-4148 | —C(═O)OMe | —CH₂CH═CCl₂ | Z-4149 | —C(═O)Et | —CH₂CH═CCl₂ |
| Z-4150 | —C(═O)—c-Pr | —CH₂CH₂CH═CF₂ | Z-4151 | —C(═O)OMe | —CH₂CH₂CH═CF₂ | Z-4152 | —C(═O)Et | —CH₂CH₂CH═CF₂ |
| Z-4153 | —C(═O)—c-Pr | —CH₂CH₂CH₂CH═CF₂ | Z-4154 | —C(═O)OMe | —CH₂CH₂CH₂CH═CF₂ | Z-4155 | —C(═O)Et | —CH₂CH₂CH₂CH═CF₂ |
| Z-4156 | —C(═O)—c-Pr | —CH₂CH₂CH₂CH₂CH═CF₂ | Z-4157 | —C(═O)OMe | —CH₂CH₂CH₂CH₂CH═CF₂ | Z-4158 | —C(═O)Et | —CH₂CH₂CH₂CH₂CH═CF₂ |
| Z-4159 | —C(═O)—c-Pr | —C≡CH | Z-4160 | —C(═O)OMe | —C≡CH | Z-4161 | —C(═O)Et | —C≡CH |
| Z-4162 | —C(═O)—c-Pr | —C≡CCH₃ | Z-4163 | —C(═O)OMe | —C≡CCH₃ | Z-4164 | —C(═O)Et | —C≡CCH₃ |
| Z-4165 | —C(═O)—c-Pr | —CH₂C≡CH | Z-4166 | —C(═O)OMe | —CH₂C≡CH | Z-4167 | —C(═O)Et | —CH₂C≡CH |
| Z-4168 | —C(═O)—c-Pr | —C≡CCH₂CH₃ | Z-4169 | —C(═O)OMe | —C≡CCH₂CH₃ | Z-4170 | —C(═O)Et | —C≡CCH₂CH₃ |
| Z-4171 | —C(═O)—c-Pr | —CH₂C≡CCH₃ | Z-4172 | —C(═O)OMe | —CH₂C≡CCH₃ | Z-4173 | —C(═O)Et | —CH₂C≡CCH₃ |
| Z-4174 | —C(═O)—c-Pr | —CH₂CH₂C≡CH | Z-4175 | —C(═O)OMe | —CH₂CH₂C≡CH | Z-4176 | —C(═O)Et | —CH₂CH₂C≡CH |
| Z-4177 | —C(═O)—c-Pr | —C≡CCH₂CH₂CH₃ | Z-4178 | —C(═O)OMe | —C≡CCH₂CH₂CH₃ | Z-4179 | —C(═O)Et | —C≡CCH₂CH₂CH₃ |
| Z-4180 | —C(═O)—c-Pr | —CH₂C≡CCH₂CH₃ | Z-4181 | —C(═O)OMe | —CH₂C≡CCH₂CH₃ | Z-4182 | —C(═O)Et | —CH₂C≡CCH₂CH₃ |
| Z-4183 | —C(═O)—c-Pr | —C(CH₃)₂C≡CH | Z-4184 | —C(═O)OMe | —C(CH₃)₂C≡CH | Z-4185 | —C(═O)Et | —C(CH₃)₂C≡CH |
| Z-4186 | —C(═O)—c-Pr | —C≡CF | Z-4187 | —C(═O)OMe | —C≡CF | Z-4188 | —C(═O)Et | —C≡CF |
| Z-4189 | —C(═O)—c-Pr | —C≡CCF₂H | Z-4190 | —C(═O)OMe | —C≡CCF₂H | Z-4191 | —C(═O)Et | —C≡CCF₂H |
| Z-4192 | —C(═O)—c-Pr | —C≡CCF₃ | Z-4193 | —C(═O)OMe | —C≡CCF₃ | Z-4194 | —C(═O)Et | —C≡CCF₃ |
| Z-4195 | —C(═O)—c-Pr | —C≡CCH₂CF₂H | Z-4196 | —C(═O)OMe | —C≡CCH₂CF₂H | Z-4197 | —C(═O)Et | —C≡CCH₂CF₂H |
| Z-4198 | —C(═O)—c-Pr | —C≡CCH₂CF₃ | Z-4199 | —C(═O)OMe | —C≡CCH₂CF₃ | Z-4200 | —C(═O)Et | —C≡CCH₂CF₃ |
| Z-4201 | —C(═O)—c-Pr | —CH₂C≡CHCF₂H | Z-4202 | —C(═O)OMe | —CH₂C≡CHCF₂H | Z-4203 | —C(═O)Et | —CH₂C≡CHCF₂H |
| Z-4204 | —C(═O)—c-Pr | —CH₂C≡CCF₃ | Z-4205 | —C(═O)OMe | —CH₂C≡CCF₃ | Z-4206 | —C(═O)Et | —CH₂C≡CCF₃ |
| Z-4207 | —C(═O)—c-Pr | —C(═O)NH₂ | Z-4208 | —C(═O)OMe | —C(═O)NH₂ | Z-4209 | —C(═O)Et | —C(═O)NH₂ |
| Z-4210 | —C(═O)—c-Pr | —C(═O)NHMe | 2-4211 | —C(═O)OMe | —C(═O)NHMe | Z-4212 | —C(═O)Et | —C(═O)NHMe |
| Z-4213 | —C(═O)—c-Pr | —C(═O)NHEt | Z-4214 | —C(═O)OMe | —C(═O)NHEt | Z-4215 | —C(═O)Et | —C(═O)NHEt |
| Z-4216 | —C(═O)—c-Pr | —C(═O)NHPr | Z-4217 | —C(═O)OMe | —C(═O)NHPr | Z-4218 | —C(═O)Et | —C(═O)NHPr |
| Z-4219 | —C(═O)—c-Pr | —C(═O)NH—i-Pr | Z-4220 | —C(═O)OMe | —C(═O)NH—i-Pr | Z-4221 | —C(═O)Et | —C(═O)NH—i-Pr |
| Z-4222 | —C(═O)—c-Pr | —C(═O)NHBu | Z-4223 | —C(═O)OMe | —C(═O)NHBu | Z-4224 | —C(═O)Et | —C(═O)NHBu |
| Z-4225 | —C(═O)—c-Pr | —C(═O)NH—sec-Bu | Z-4226 | —C(═O)OMe | —C(═O)NH—sec-Bu | Z-4227 | —C(═O)Et | —C(═O)NH—sec-Bu |
| Z-4228 | —C(═O)—c-Pr | —C(═O)NH—i-Bu | Z-4229 | —C(═O)OMe | —C(═O)NH—i-Bu | Z-4230 | —C(═O)Et | —C(═O)NH—i-Bu |
| Z-4231 | —C(═O)—c-Pr | —C(═O)NH—t-Bu | Z-4232 | —C(═O)OMe | —C(═O)NH—t-Bu | Z-4233 | —C(═O)Et | —C(═O)NH—t-Bu |
| Z-4234 | —C(═O)—c-Pr | —C(═O)NHPent | Z-4235 | —C(═O)OMe | —C(═O)NHPent | Z-4236 | —C(═O)Et | —C(═O)NHPent |
| Z-4237 | —C(═O)—c-Pr | —C(═O)NHHex | Z-4238 | —C(═O)OMe | —C(═O)NHHex | Z-4239 | —C(═O)Et | —C(═O)NHHex |
| Z-4240 | —C(═O)—c-Pr | —C(═O)NHCH₂C≡N | Z-4241 | —C(═O)OMe | —C(═O)NHCH₂C≡N | Z-4242 | —C(═O)Et | —C(═O)NHCH₂C≡N |
| Z-4243 | —C(═O)—c-Pr | —C(═O)NHCH₂—c-Pr | Z-4244 | —C(═O)OMe | —C(═O)NHCH₂—c-Pr | Z-4245 | —C(═O)Et | —C(═O)NHCH₂—c-Pr |
| Z-4246 | —C(═O)—c-Pr | —C(═O)NHCH₂OMe | Z-4247 | —C(═O)OMe | —C(═O)NHCH₂OMe | Z-4248 | —C(═O)Et | —C(═O)NHCH₂OMe |
| Z-4249 | —C(═O)—c-Pr | —C(═O)NHCH₂CH₂OMe | Z-4250 | —C(═O)OMe | —C(═O)NHCH₂CH₂OMe | Z-4251 | —C(═O)Et | —C(═O)NHCH₂CH₂OMe |
| Z-4252 | —C(═O)—c-Pr | —C(═O)NHCFH₂ | Z-4253 | —C(═O)OMe | —C(═O)NHCFH₂ | Z-4254 | —C(═O)Et | —C(═O)NHCFH₂ |
| Z-4255 | —C(═O)—c-Pr | —C(═O)NHCF₂H | Z-4256 | —C(═O)OMe | —C(═O)NHCF₂H | Z-4257 | —C(═O)Et | —C(═O)NHCF₂H |
| Z-4258 | —C(═O)—c-Pr | —C(═O)NHCF₃ | Z-4259 | —C(═O)OMe | —C(═O)NHCF₃ | Z-4260 | —C(═O)Et | —C(═O)NHCF₃ |
| Z-4261 | —C(═O)—c-Pr | —C(═O)NHCH₂Cl | Z-4262 | —C(═O)OMe | —C(═O)NHCH₂Cl | Z-4263 | —C(═O)Et | —C(═O)NHCH₂Cl |
| Z-4264 | —C(═O)—c-Pr | —C(═O)NHCHCl₂ | Z-4265 | —C(═O)OMe | —C(═O)NHCHCl₂ | Z-4266 | —C(═O)Et | —C(═O)NHCHCl₂ |
| Z-4267 | —C(═O)—c-Pr | —C(═O)NHCCl₃ | Z-4268 | —C(═O)OMe | —C(═O)NHCCl₃ | Z-4269 | —C(═O)Et | —C(═O)NHCCl₃ |
| Z-4270 | —C(═O)—c-Pr | —C(═O)NHCH₂Br | Z-4271 | —C(═O)OMe | —C(═O)NHCH₂Br | Z-4272 | —C(═O)Et | —C(═O)NHCH₂Br |
| Z-4273 | —C(═O)—c-Pr | —C(═O)NHCHBr₂ | Z-4274 | —C(═O)OMe | —C(═O)NHCHBr₂ | Z-4275 | —C(═O)Et | —C(═O)NHCHBr₂ |
| Z-4276 | —C(═O)—c-Pr | —C(═O)NHCBr₃ | Z-4277 | —C(═O)OMe | —C(═O)NHCBr₃ | Z-4278 | —C(═O)Et | —C(═O)NHCBr₃ |
| Z-4279 | —C(═O)—c-Pr | —C(═O)NHCH₂l | Z-4280 | —C(═O)OMe | —C(═O)NHCH₂l | Z-4281 | —C(═O)Et | —C(═O)NHCH₂l |
| Z-4282 | —C(═O)—c-Pr | —C(═O)NHCHl₂ | Z-4283 | —C(═O)OMe | —C(═O)NHCHl₂ | Z-4284 | —C(═O)Et | —C(═O)NHCHl₂ |
| Z-4285 | —C(═O)—c-Pr | —C(═O)NHCH₂CF₂H | Z-4286 | —C(═O)OMe | —C(═O)NHCH₂CF₂H | Z-4287 | —C(═O)Et | —C(═O)NHCH₂CF₂H |
| Z-4288 | —C(═O)—c-Pr | —C(═O)NHCH₂CF₃ | Z-4289 | —C(═O)OMe | —C(═O)NHCH₂CF₃ | Z-4290 | —C(═O)Et | —C(═O)NHCH₂CF₃ |
| Z-4291 | —C(═O)—c-Pr | —C(═O)NHCH₂CH₂CF₂H | Z-4292 | —C(═O)OMe | —C(═O)NHCH₂CH₂CF₂H | Z-4293 | —C(═O)Et | —C(═O)NHCH₂CH₂CF₂H |
| Z-4294 | —C(═O)—c-Pr | —C(═O)NHCH₂CH₂CF₃ | Z-4295 | —C(═O)OMe | —C(═O)NHCH₂CH₂CF₃ | Z-4296 | —C(═O)Et | —C(═O)NHCH₂CH₂CF₃ |
| Z-4297 | —C(═O)—c-Pr | —C(═O)NHCH₂CH₂CH₂CF₂H | Z-4298 | —C(═O)OMe | —C(═O)NHCH₂CH₂CH₂CF₂H | Z-4299 | —C(═O)Et | —C(═O)NHCH₂CH₂CH₂CF₂H |
| Z-4300 | —C(═O)—c-Pr | —C(═O)NHCH₂CH₂CH₂CF₃ | Z-4301 | —C(═O)OMe | —C(═O)NHCH₂CH₂CH₂CF₃ | Z-4302 | —C(═O)Et | —C(═O)NHCH₂CH₂CH₂CF₃ |
| Z-4303 | —C(═O)—c-Pr | —C(═O)NHCF₂CF₂H | Z-4304 | —C(═O)OMe | —C(═O)NHCF₂CF₂H | Z-4305 | —C(═O)Et | —C(═O)NHCF₂CF₂H |
| Z-4306 | —C(═O)—c-Pr | —C(═O)NHCF₂CF₃ | Z-4307 | —C(═O)OMe | —C(═O)NHCF₂CF₃ | Z-4308 | —C(═O)Et | —C(═O)NHCF₂CF₃ |
| Z-4309 | —C(═O)—c-Pr | —C(═O)NHCFHCF₃ | Z-4310 | —C(═O)OMe | —C(═O)NHCFHCF₃ | Z-4311 | —C(═O)Et | —C(═O)NHCFHCF₃ |
| Z-4312 | —C(═O)—c-Pr | —C(═O)NHCH₂CF₂CF₂H | Z-4313 | —C(═O)OMe | —C(═O)NHCH₂CF₂CF₂H | Z-4314 | —C(═O)Et | —C(═O)NHCH₂CF₂CF₂H |
| Z-4315 | —C(═O)—c-Pr | —C(═O)NHCH₂CF₂CF₃ | Z-4316 | —C(═O)OMe | —C(═O)NHCH₂CF₂CF₃ | Z-4317 | —C(═O)Et | —C(═O)NHCH₂CF₂CF₃ |
| Z-4318 | —C(═O)—c-Pr | —C(═O)NHCF₂CF₂CF₃ | Z-4319 | —C(═O)OMe | —C(═O)NHCF₂CF₂CF₃ | Z-4320 | —C(═O)Et | —C(═O)NHCF₂CF₂CF₃ |
| Z-4321 | —C(═O)—c-Pr | —C(═O)NHCH₂CF₂CF₂CF₃ | Z-4322 | —C(═O)OMe | —C(═O)NHCH₂CF₂CF₂CF₃ | Z-4323 | —C(═O)Et | —C(═O)NHCH₂CF₂CF₂CF₃ |
| Z-4324 | —C(═O)—c-Pr | —C(═O)NHCF₂CF₂CF₂CF₃ | Z-4325 | —C(═O)OMe | —C(═O)NHCF₂CF₂CF₂CF₃ | Z-4326 | —C(═O)Et | —C(═O)NHCF₂CF₂CF₂CF₃ |
| Z-4327 | —C(═O)—c-Pr | —C(═O)NHCH₂CF₂CF₂CF₂CF₃ | Z-4328 | —C(═O)OMe | —C(═O)NHCH₂CF₂CF₂CF₂CF₃ | Z-4329 | —C(═O)Et | —C(═O)NHCH₂CF₂CF₂CF₂CF₃ |
| Z-4330 | —C(═O)—c-Pr | —C(═O)NH—c-Pr | Z-4331 | —C(═O)OMe | —C(═O)NH—c-Pr | Z-4332 | —C(═O)Et | —C(═O)NH—c-Pr |
| Z-4333 | —C(═O)—c-Pr | —C(═O)NH—c-Bu | Z-4334 | —C(═O)OMe | —C(═O)NH—c-Bu | Z-4335 | —C(═O)Et | —C(═O)NH—c-Bu |
| Z-4336 | —C(═O)—c-Pr | —C(═O)NH—c-Pent | Z-4337 | —C(═O)OMe | —C(═O)NH—c-Pent | Z-4338 | —C(═O)Et | —C(═O)NH—c-Pent |
| Z-4339 | —C(═O)—c-Pr | —C(═O)NH—c-Hex | Z-4340 | —C(═O)OMe | —C(═O)NH—c-Hex | Z-4341 | —C(═O)Et | —C(═O)NH—c-Hex |
| Z-4342 | —C(═O)—c-Pr | —C(═O)NH—c-Hept | Z-4343 | —C(═O)OMe | —C(═O)NH—c-Hept | Z-4344 | —C(═O)Et | —C(═O)NH—c-Hept |
| Z-4345 | —C(═O)—c-Pr | —C(═O)NH—c-Oct | Z-4346 | —C(═O)OMe | —C(═O)NH—c-Oct | Z-4347 | —C(═O)Et | —C(═O)NH—c-Oct |
| Z-4348 | —C(═O)—c-Pr | —C(═O)NHCH₂CH═CH₂ | Z-4349 | —C(═O)OMe | —C(═O)NHCH₂CH═CH₂ | Z-4350 | —C(═O)Et | —C(═O)NHCH₂CH═CH₂ |
| Z-4351 | —C(═O)—c-Pr | —C(═O)NHCH₂C(CH₃)═CH₂ | Z-4352 | —C(═O)OMe | —C(═O)NHCH₂C(CH₃)═CH₂ | Z-4353 | —C(═O)Et | —C(═O)NHCH₂C(CH₃)═CH₂ |
| Z-4354 | —C(═O)—c-Pr | —C(═O)NHCH₂CH₂CH═CH₂ | Z-4355 | —C(═O)OMe | —C(═O)NHCH₂CH₂CH═CH₂ | Z-4356 | —C(═O)Et | —C(═O)NHCH₂CH₂CH═CH₂ |
| Z-4357 | —C(═O)—c-Pr | —C(═O)NHCH₂CH═CHCH₃ | Z-4358 | —C(═O)OMe | —C(═O)NHCH₂CH═CHCH₃ | Z-4359 | —C(═O)Et | —C(═O)NHCH₂CH═CHCH₃ |
| Z-4360 | —C(═O)—c-Pr | —C(═O)NHCH₂CH═C(CH₃)₂ | Z-4361 | —C(═O)OMe | —C(═O)NHCH₂CH═C(CH₃)₂ | Z-4362 | —C(═O)Et | —C(═O)NHCH₂CH═C(CH₃)₂ |
| Z-4363 | —C(═O)—c-Pr | —C(═O)NHCH₂CH₂CH═C(CH₃)₂ | Z-4364 | —C(═O)OMe | —C(═O)NHCH₂CH₂CH═C(CH₃)₂ | Z-4365 | —C(═O)Et | —C(═O)NHCH₂CH₂CH═C(CH₃)₂ |
| Z-4366 | —C(═O)—c-Pr | —C(═O)NHCH₂CH═CFH | Z-4367 | —C(═O)OMe | —C(═O)NHCH₂CH═CFH | Z-4368 | —C(═O)Et | —C(═O)NHCH₂CH═CFH |
| Z-4369 | —C(═O)—c-Pr | —C(═O)NHCH₂CH═CF₂ | Z-4370 | —C(═O)OMe | —C(═O)NHCH₂CH═CF₂ | Z-4371 | —C(═O)Et | —C(═O)NHCH₂CH═CF₂ |
| Z-4372 | —C(═O)—c-Pr | —C(═O)NHCH₂CH═CCl₂ | Z-4373 | —C(═O)OMe | —C(═O)NHCH₂CH═CCl₂ | Z-4374 | —C(═O)Et | —C(═O)NHCH₂CH═CCl₂ |
| Z-4375 | —C(═O)—c-Pr | —C(═O)NHCH₂CH₂CH═CF₂ | Z-4376 | —C(═O)OMe | —C(═O)NHCH₂CH₂CH═CF₂ | Z-4377 | —C(═O)Et | —C(═O)NHCH₂CH₂CH═CF₂ |
| Z-4378 | —C(═O)—c-Pr | —C(═O)NHCH₂CH₂CH₂CH═CF₂ | Z-4379 | —C(═O)OMe | —C(═O)NHCH₂CH₂CH₂CH═CF₂ | Z-4380 | —C(═O)Et | —C(═O)NHCH₂CH₂CH₂CH═CF₂ |
| Z-4381 | —C(═O)—c-Pr | —C(═O)NHCH₂CH₂CH₂CH₂CH═CF₂ | Z-4382 | —C(═O)OMe | —C(═O)NHCH₂CH₂CH₂CH₂CH═CF₂ | Z-4383 | —C(═O)Et | —C(═O)NHCH₂CH₂CH₂CH₂CH═CF₂ |
| Z-4384 | —C(═O)—c-Pr | —C(═O)NHCH₂C≡CH | Z-4385 | —C(═O)OMe | —C(═O)NHCH₂C≡CH | Z-4386 | —C(═O)Et | —C(═O)NHCH₂C≡CH |
| Z-4387 | —C(═O)—c-Pr | —C(═O)NHCH₂C≡CCH₃ | Z-4388 | —C(═O)OMe | —C(═O)NHCH₂C≡CCH₃ | Z-4389 | —C(═O)Et | —C(═O)NHCH₂C≡CCH₃ |
| Z-4390 | —C(═O)—c-Pr | —C(═O)NHCH₂CH₂C≡CH | Z-4391 | —C(═O)OMe | —C(═O)NHCH₂CH₂C≡CH | Z-4392 | —C(═O)Et | —C(═O)NHCH₂CH₂C≡CH |
| Z-4393 | —C(═O)—c-Pr | —C(═O)NHCH₂C≡CCH₂CH₃ | Z-4394 | —C(═O)OMe | —C(═O)NHCH₂C≡CCH₂CH₃ | Z-4395 | —C(═O)Et | —C(═O)NHCH₂C≡CCH₂CH₃ |
| Z-4396 | —C(═O)—c-Pr | —C(═O)NHC(CH₃)₂C≡CH | Z-4397 | —C(═O)OMe | —C(═O)NHC(CH₃)₂C≡CH | Z-4398 | —C(═O)Et | —C(═O)NHC(CH₃)₂C≡CH |
| Z-4399 | —C(═O)—c-Pr | —C(═O)NHCH₂C≡CHCF₂H | Z-4400 | —C(═O)OMe | —C(═O)NHCH₂C≡CHCF₂H | Z-4401 | —C(═O)Et | —C(═O)NHCH₂C≡CHCF₂H |
| Z-4402 | —C(═O)—c-Pr | —C(═O)NHCH₂C≡CCF₃ | Z-4403 | —C(═O)OMe | —C(═O)NHCH₂C≡CCF₃ | Z-4404 | —C(═O)Et | —C(═O)NHCH₂C≡CCF₃ |
| Z-4405 | —C(═O)—c-Pr | —C(═O)H | Z-4406 | —C(═O)OMe | —C(═O)H | Z-4407 | —C(═O)Et | —C(═O)H |
| Z-4408 | —C(═O)—c-Pr | —C(═O)Me | Z-4409 | —C(═O)OMe | —C(═O)Me | Z-4410 | —C(═O)Et | —C(═O)Me |
| Z-4411 | —C(═O)—c-Pr | —C(═O)Et | Z-4412 | —C(═O)OMe | —C(═O)Et | Z-4413 | —C(═O)Et | —C(═O)Et |
| Z-4414 | —C(═O)—c-Pr | —C(═O)Pr | Z-4415 | —C(═O)OMe | —C(═O)Pr | Z-4416 | —C(═O)Et | —C(═O)Pr |
| Z-4417 | —C(═O)—c-Pr | —C(═O)—i-Pr | Z-4418 | —C(═O)OMe | —C(═O)—i-Pr | Z-4419 | —C(═O)Et | —C(═O)—i-Pr |
| Z-4420 | —C(═O)—c-Pr | —C(═O)Bu | Z-4421 | —C(═O)OMe | —C(═O)Bu | Z-4422 | —C(═O)Et | —C(═O)Bu |
| Z-4423 | —C(═O)—c-Pr | —C(═O)—sec-Bu | Z-4424 | —C(═O)OMe | —C(═O)—sec-Bu | Z-4425 | —C(═O)Et | —C(═O)—sec-Bu |
| Z-4426 | —C(═O)—c-Pr | —C(═O)—i-Bu | Z-4427 | —C(═O)OMe | —C(═O)—i-Bu | Z-4428 | —C(═O)Et | —C(═O)—i-Bu |
| Z-4429 | —C(═O)—c-Pr | —C(═O)—t-Bu | Z-4430 | —C(═O)OMe | —C(═O)—t-Bu | Z-4431 | —C(═O)Et | —C(═O)—t-Bu |
| Z-4432 | —C(═O)—c-Pr | —C(═O)Pent | Z-4433 | —C(═O)OMe | —C(═O)Pent | Z-4434 | —C(═O)Et | —C(═O)Pent |
| Z-4435 | —C(═O)—c-Pr | —C(═O)Hex | Z-4436 | —C(═O)OMe | —C(═O)Hex | Z-4437 | —C(═O)Et | —C(═O)Hex |
| Z-4438 | —C(═O)—c-Pr | —C(═O)CH(CH₃)CH₂CH₂CH₃ | Z-4439 | —C(═O)OMe | —C(═O)CH(CH₃)CH₂CH₂CH₃ | Z-4440 | —C(═O)Et | —C(═O)CH(CH₃)CH₂CH₂CH₃ |
| Z-4441 | —C(═O)—c-Pr | —C(═O)CH(CH₃)CH(CH₃)₂ | Z-4442 | —C(═O)OMe | —C(═O)CH(CH₃)CH(CH₃)₂ | Z-4443 | —C(═O)Et | —C(═O)CH(CH₃)CH(CH₃)₂ |
| Z-4444 | —C(═O)—c-Pr | —C(═O)C(CH₃)₂CH₂CH₃ | Z-4445 | —C(═O)OMe | —C(═O)C(CH₃)₂CH₂CH₃ | Z-4446 | —C(═O)Et | —C(═O)C(CH₃)₂CH₂CH₃ |
| Z-4447 | —C(═O)—c-Pr | —C(═O)CH(CH₂CH₃)₂ | Z-4448 | —C(═O)OMe | —C(═O)CH(CH₂CH₃)₂ | Z-4449 | —C(═O)Et | —C(═O)CH(CH₂CH₃)₂ |
| Z-4450 | —C(═O)—c-Pr | —C(═O)CH₂CH₂CH(CH₃)₂ | Z-4451 | —C(═O)OMe | —C(═O)CH₂CH₂CH(CH₃)₂ | Z-4452 | —C(═O)Et | —C(═O)CH₂CH₂CH(CH₃)₂ |
| Z-4453 | —C(═O)—c-Pr | —C(═O)CH₂CH₂CH₂CH(CH₃)₂ | Z-4454 | —C(═O)OMe | —C(═O)CH₂CH₂CH₂CH(CH₃)₂ | Z-4455 | —C(═O)Et | —C(═O)CH₂CH₂CH₂CH(CH₃)₂ |
| Z-4456 | —C(═O)—c-Pr | —C(═O)CH₂CH₂CH(CH₃)CH₂CH₃ | Z-4457 | —C(═O)OMe | —C(═O)CH₂CH₂CH(CH₃)CH₂CH₃ | Z-4458 | —C(═O)Et | —C(═O)CH₂CH₂CH(CH₃)CH₂CH₃ |
| Z-4459 | —C(═O)—c-Pr | —C(═O)CH₂CH(CH₃)CH₂CH₂CH₃ | Z-4460 | —C(═O)OMe | —C(═O)CH₂CH(CH₃)CH₂CH₂CH₃ | Z-4461 | —C(═O)Et | —C(═O)CH₂CH(CH₃)CH₂CH₂CH₃ |
| Z-4462 | —C(═O)—c-Pr | —C(═O)CH(CH₃)CH₂CH₂CH₂CH₃ | Z-4463 | —C(═O)OMe | —C(═O)CH(CH₃)CH₂CH₂CH₂CH₃ | Z-4464 | —C(═O)Et | —C(═O)CH(CH₃)CH₂CH₂CH₂CH₃ |
| Z-4465 | —C(═O)—c-Pr | —C(═O)CH(CH₃)CH₂CH(CH₃)₂ | Z-4466 | —C(═O)OMe | —C(═O)CH(CH₃)CH₂CH(CH₃)₂ | Z-4467 | —C(═O)Et | —C(═O)CH(CH₃)CH₂CH(CH₃)₂ |
| Z-4468 | —C(═O)—c-Pr | —C(═O)CH(CH₃)CH(CH₃)CH₂CH₃ | Z-4469 | —C(═O)OMe | —C(═O)CH(CH₃)CH(CH₃)CH₂CH₃ | Z-4470 | —C(═O)Et | —C(═O)CH(CH₃)CH(CH₃)CH₂CH₃ |
| Z-4471 | —C(═O)—c-Pr | —C(═O)C(CH₃)₂CH₂CH₂CH₃ | Z-4472 | —C(═O)OMe | —C(═O)C(CH₃)₂CH₂CH₂CH₃ | Z-4473 | —C(═O)Et | —C(═O)C(CH₃)₂CH₂CH₂CH₃ |
| Z-4474 | —C(═O)—c-Pr | —C(═O)CH(CH₃)C(CH₃)₃ | Z-4475 | —C(═O)OMe | —C(═O)CH(CH₃)C(CH₃)₃ | Z-4476 | —C(═O)Et | —C(═O)CH(CH₃)C(CH₃)₃ |
| Z-4477 | —C(═O)—c-Pr | —C(═O)C(CH₃)₂CH(CH₃)₂ | Z-4478 | —C(═O)OMe | —C(═O)C(CH₃)₂CH(CH₃)₂ | Z-4479 | —C(═O)Et | —C(═O)C(CH₃)₂CH(CH₃)₂ |
| Z-4480 | —C(═O)—c-Pr | —C(═O)CH₂CH₂C(CH₃) | Z-4481 | —C(═O)OMe | —C(═O)CH₂CH₂C(CH₃) | Z-4482 | —C(═O)Et | —C(═O)CH₂CH₂C(CH₃) |
| Z-4483 | —C(═O)—c-Pr | —C(═O)CH₂CH(CH₃)CH(CH₃)₂ | Z-4484 | —C(═O)OMe | —C(═O)CH₂CH(CH₃)CH(CH₃)₂ | Z-4485 | —C(═O)Et | —C(═O)CH₂CH(CH₃)CH(CH₃)₂ |
| Z-4486 | —C(═O)—c-Pr | —C(═O)CH₂C(CH₃)₂CH₂CH₃ | Z-4487 | —C(═O)OMe | —C(═O)CH₂C(CH₃)₂CH₂CH₃ | Z-4488 | —C(═O)Et | —C(═O)CH₂C(CH₃)₂CH₂CH₃ |
| Z-4489 | —C(═O)—c-Pr | —C(═O)CFH₂ | Z-4490 | —C(═O)OMe | —C(═O)CFH₂ | Z-4491 | —C(═O)Et | —C(═O)CFH₂ |
| Z-4492 | —C(═O)—c-Pr | —C(═O)CF₂H | Z-4493 | —C(═O)OMe | —C(═O)CF₂H | Z-4494 | —C(═O)Et | —C(═O)CF₂H |
| Z-4495 | —C(═O)—c-Pr | —C(═O)CF₂Cl | Z-4496 | —C(═O)OMe | —C(═O)CF₂Cl | Z-4497 | —C(═O)Et | —C(═O)CF₂Cl |
| Z-4498 | —C(═O)—c-Pr | —C(═O)CF₃ | Z-4499 | —C(═O)OMe | —C(═O)CF₃ | Z-4500 | —C(═O)Et | —C(═O)CF₃ |
| Z-4501 | —C(═O)—c-Pr | —C(═O)CH₂Cl | Z-4502 | —C(═O)OMe | —C(═O)CH₂Cl | Z-4503 | —C(═O)Et | —C(═O)CH₂Cl |
| Z-4504 | —C(═O)—c-Pr | —C(═O)CHCl₂ | Z-4505 | —C(═O)OMe | —C(═O)CHCl₂ | Z-4506 | —C(═O)Et | —C(═O)CHCl₂ |
| Z-4507 | —C(═O)—c-Pr | —C(═O)CCl₂F | Z-4508 | —C(═O)OMe | —C(═O)CCl₂F | Z-4509 | —C(═O)Et | —C(═O)CCl₂F |
| Z-4510 | —C(═O)—c-Pr | —C(═O)CCl₃ | Z-4511 | —C(═O)OMe | —C(═O)CCl₃ | Z-4512 | —C(═O)Et | —C(═O)CCl₃ |
| Z-4513 | —C(═O)—c-Pr | —C(═O)CH₂Br | Z-4514 | —C(═O)OMe | —C(═O)CH₂Br | Z-4515 | —C(═O)Et | —C(═O)CH₂Br |
| Z-4516 | —C(═O)—c-Pr | —C(═O)CHBr₂ | Z-4517 | —C(═O)OMe | —C(═O)CHBr₂ | Z-4518 | —C(═O)Et | —C(═O)CHBr₂ |
| Z-4519 | —C(═O)—c-Pr | —C(═O)CBr₃ | Z-4520 | —C(═O)OMe | —C(═O)CBr₃ | Z-4521 | —C(═O)Et | —C(═O)CBr₃ |
| Z-4522 | —C(═O)—c-Pr | —C(═O)CH₂l | Z-4523 | —C(═O)OMe | —C(═O)CH₂l | Z-4524 | —C(═O)Et | —C(═O)CH₂l |
| Z-4525 | —C(═O)—c-Pr | —C(═O)CHl₂ | Z-4526 | —C(═O)OMe | —C(═O)CHl₂ | Z-4527 | —C(═O)Et | —C(═O)CHl₂ |
| Z-4528 | —C(═O)—c-Pr | —C(═O)CH₂CF₂H | Z-4529 | —C(═O)OMe | —C(═O)CH₂CF₂H | Z-4530 | —C(═O)Et | —C(═O)CH₂CF₂H |
| Z-4531 | —C(═O)—c-Pr | —C(═O)CH₂CF₃ | Z-4532 | —C(═O)OMe | —C(═O)CH₂CF₃ | Z-4533 | —C(═O)Et | —C(═O)CH₂CF₃ |
| Z-4534 | —C(═O)—c-Pr | —C(═O)CH₂CH₂CF₂H | Z-4535 | —C(═O)OMe | —C(═O)CH₂CH₂CF₂H | Z-4536 | —C(═O)Et | —C(═O)CH₂CH₂CF₂H |
| Z-4537 | —C(═O)—c-Pr | —C(═O)CH₂CH₂CF₃ | Z-4538 | —C(═O)OMe | —C(═O)CH₂CH₂CF₃ | Z-4539 | —C(═O)Et | —C(═O)CH₂CH₂CF₃ |
| Z-4540 | —C(═O)—c-Pr | —C(═O)CH₂CH₂CH₂CF₂H | Z-4541 | —C(═O)OMe | —C(═O)CH₂CH₂CH₂CF₂H | Z-4542 | —C(═O)Et | —C(═O)CH₂CH₂CH₂CF₂H |
| Z-4543 | —C(═O)—c-Pr | —C(═O)CH₂CH₂CH₂CF₃ | Z-4544 | —C(═O)OMe | —C(═O)CH₂CH₂CH₂CF₃ | Z-4545 | —C(═O)Et | —C(═O)CH₂CH₂CH₂CF₃ |
| Z-4546 | —C(═O)—c-Pr | —C(═O)CF₂CH₃ | Z-4547 | —C(═O)OMe | —C(═O)CF₂CH₃ | Z-4548 | —C(═O)Et | —C(═O)CF₂CH₃ |
| Z-4549 | —C(═O)—c-Pr | —C(═O)CF₂CF₂H | Z-4550 | —C(═O)OMe | —C(═O)CF₂CF₂H | Z-4551 | —C(═O)Et | —C(═O)CF₂CF₂H |
| Z-4552 | —C(═O)—c-Pr | —C(═O)CF₂CF₃ | Z-4553 | —C(═O)OMe | —C(═O)CF₂CF₃ | Z-4554 | —C(═O)Et | —C(═O)CF₂CF₃ |
| Z-4555 | —C(═O)—c-Pr | —C(═O)CF₂CClF₂ | Z-4556 | —C(═O)OMe | —C(═O)CF₂CClF₂ | Z-4557 | —C(═O)Et | —C(═O)CF₂CClF₂ |
| Z-4558 | —C(═O)—c-Pr | —C(═O)CFHCF₃ | Z-4559 | —C(═O)OMe | —C(═O)CFHCF₃ | Z-4560 | —C(═O)Et | —C(═O)CFHCF₃ |
| Z-4561 | —C(═O)—c-Pr | —C(═O)CH₂CF₂CF₂H | Z-4562 | —C(═O)OMe | —C(═O)CH₂CF₂CF₂H | Z-4563 | —C(═O)Et | —C(═O)CH₂CF₂CF₂H |
| Z-4564 | —C(═O)—c-Pr | —C(═O)CH₂CF₂CF₃ | Z-4565 | —C(═O)OMe | —C(═O)CH₂CF₂CF₃ | Z-4566 | —C(═O)Et | —C(═O)CH₂CF₂CF₃ |
| Z-4567 | —C(═O)—c-Pr | —C(═O)CF₂CF₂CF₃ | Z-4568 | —C(═O)OMe | —C(═O)CF₂CF₂CF₃ | Z-4569 | —C(═O)Et | —C(═O)CF₂CF₂CF₃ |
| Z-4570 | —C(═O)—c-Pr | —C(═O)CH₂CF₂CF₂CF₃ | Z-4571 | —C(═O)OMe | —C(═O)CH₂CF₂CF₂CF₃ | Z-4572 | —C(═O)Et | —C(═O)CH₂CF₂CF₂CF₃ |
| Z-4573 | —C(═O)—c-Pr | —C(═O)CF₂CF₂CF₂CF₃ | Z-4574 | —C(═O)OMe | —C(═O)CF₂CF₂CF₂CF₃ | Z-4575 | —C(═O)Et | —C(═O)CF₂CF₂CF₂CF₃ |
| Z-4576 | —C(═O)—c-Pr | —C(═O)CH₂CF₂CF₂CF₂CF₃ | Z-4577 | —C(═O)OMe | —C(═O)CH₂CF₂CF₂CF₂CF₃ | Z-4578 | —C(═O)Et | —C(═O)CH₂CF₂CF₂CF₂CF₃ |
| Z-4579 | —C(═O)—c-Pr | —C(═O)CF₂CF₂CF₂CF₂CF₃ | Z-4580 | —C(═O)OMe | —C(═O)CF₂CF₂CF₂CF₂CF₃ | Z-4581 | —C(═O)Et | —C(═O)CF₂CF₂CF₂CF₂CF₃ |
| Z-4582 | —C(═O)—c-Pr | —C(═O)—c-Pr | Z-4583 | —C(═O)OMe | —C(═O)—c-Pr | Z-4584 | —C(═O)Et | —C(═O)—c-Pr |
| Z-4585 | —C(═O)—c-Pr | —C(═O)—c-Bu | Z-4586 | —C(═O)OMe | —C(═O)—c-Bu | Z-4587 | —C(═O)Et | —C(═O)—c-Bu |
| Z-4588 | —C(═O)—c-Pr | —C(═O)—c-Pent | Z-4589 | —C(═O)OMe | —C(═O)—c-Pent | Z-4590 | —C(═O)Et | —C(═O)—c-Pent |
| Z-4591 | —C(═O)—c-Pr | —C(═O)—c-Hex | Z-4592 | —C(═O)OMe | —C(═O)—c-Hex | Z-4593 | —C(═O)Et | —C(═O)—c-Hex |
| Z-4594 | —C(═O)—c-Pr | —C(═O)—c-Hept | Z-4595 | —C(═O)OMe | —C(═O)—c-Hept | Z-4596 | —C(═O)Et | —C(═O)—c-Hept |
| Z-4597 | —C(═O)—c-Pr | —C(═O)—c-Oct | Z-4598 | —C(═O)OMe | —C(═O)—c-Oct | Z-4599 | —C(═O)Et | —C(═O)—c-Oct |
| Z-4600 | —C(═O)—c-Pr | —C(═O)CH═CH₂ | Z-4601 | —C(═O)OMe | —C(═O)CH═CH₂ | Z-4602 | —C(═O)Et | —C(═O)CH═CH₂ |
| Z-4603 | —C(═O)—c-Pr | —C(═O)CH₂CH═CH₂ | Z-4604 | —C(═O)OMe | —C(═O)CH₂CH═CH₂ | Z-4605 | —C(═O)Et | —C(═O)CH₂CH═CH₂ |
| Z-4606 | —C(═O)—c-Pr | —C(═O)CH═CHCH₃ | Z-4607 | —C(═O)OMe | —C(═O)CH═CHCH₃ | Z-4608 | —C(═O)Et | —C(═O)CH═CHCH₃ |
| Z-4609 | —C(═O)—c-Pr | —C(═O)CH₂C(CH₃)═CH₂ | Z-4610 | —C(═O)OMe | —C(═O)CH₂C(CH₃)═CH₂ | Z-4611 | —C(═O)Et | —C(═O)CH₂C(CH₃)═CH₂ |
| Z-4612 | —C(═O)—c-Pr | —C(═O)CH₂CH₂CH═CH₂ | Z-4613 | —C(═O)OMe | —C(═O)CH₂CH₂CH═CH₂ | Z-4614 | —C(═O)Et | —C(═O)CH₂CH₂CH═CH₂ |
| Z-4615 | —C(═O)—c-Pr | —C(═O)CH₂CH═CHCH₃ | Z-4616 | —C(═O)OMe | —C(═O)CH₂CH═CHCH₃ | Z-4617 | —C(═O)Et | —C(═O)CH₂CH═CHCH₃ |
| Z-4618 | —C(═O)—c-Pr | —C(═O)CH═CHCH₂CH₃ | Z-4619 | —C(═O)OMe | —C(═O)CH═CHCH₂CH₃ | Z-4620 | —C(═O)Et | —C(═O)CH═CHCH₂CH₃ |
| Z-4621 | —C(═O)—c-Pr | —C(═O)CH₂CH═C(CH₃)₂ | Z-4622 | —C(═O)OMe | —C(═O)CH₂CH═C(CH₃)₂ | Z-4623 | —C(═O)Et | —C(═O)CH₂CH═C(CH₃)₂ |
| Z-4624 | —C(═O)—c-Pr | —C(═O)CH₂CH₂CH═C(CH₃)₂ | Z-4625 | —C(═O)OMe | —C(═O)CH₂CH₂CH═C(CH₃)₂ | Z-4626 | —C(═O)Et | —C(═O)CH₂CH₂CH═C(CH₃)₂ |
| Z-4627 | —C(═O)—c-Pr | —C(═O)CH═CFH | Z-4628 | —C(═O)OMe | —C(═O)CH═CFH | Z-4629 | —C(═O)Et | —C(═O)CH═CFH |
| Z-4630 | —C(═O)—c-Pr | —C(═O)CH═CF₂ | Z-4631 | —C(═O)OMe | —C(═O)CH═CF₂ | Z-4632 | —C(═O)Et | —C(═O)CH═CF₂ |
| Z-4633 | —C(═O)—c-Pr | —C(═O)CH═CCl₂ | Z-4634 | —C(═O)OMe | —C(═O)CH═CCl₂ | Z-4635 | —C(═O)Et | —C(═O)CH═CCl₂ |
| Z-4636 | —C(═O)—c-Pr | —C(═O)CH₂CH═CFH | Z-4637 | —C(═O)OMe | —C(═O)CH₂CH═CFH | Z-4638 | —C(═O)Et | —C(═O)CH₂CH═CFH |
| Z-4639 | —C(═O)—c-Pr | —C(═O)CH₂CH═CF₂ | Z-4640 | —C(═O)OMe | —C(═O)CH₂CH═CF₂ | Z-4641 | —C(═O)Et | —C(═O)CH₂CH═CF₂ |
| Z-4642 | —C(═O)—c-Pr | —C(═O)CH₂CH═CCl₂ | Z-4643 | —C(═O)OMe | —C(═O)CH₂CH═CCl₂ | Z-4644 | —C(═O)Et | —C(═O)CH₂CH═CCl₂ |
| Z-4645 | —C(═O)—c-Pr | —C(═O)CH₂CH₂CH═CF₂ | Z-4646 | —C(═O)OMe | —C(═O)CH₂CH₂CH═CF₂ | Z-4647 | —C(═O)Et | —C(═O)CH₂CH₂CH═CF₂ |
| Z-4648 | —C(═O)—c-Pr | —C(═O)CH₂CH₂CH₂CH═CF₂ | Z-4649 | —C(═O)OMe | —C(═O)CH₂CH₂CH₂CH═CF₂ | Z-4650 | —C(═O)Et | —C(═O)CH₂CH₂CH₂CH═CF₂ |
| Z-4651 | —C(═O)—c-Pr | —C(═O)CH₂CH₂CH₂CH₂CH═CF₂ | Z-4652 | —C(═O)OMe | —C(═O)CH₂CH₂CH₂CH₂CH═CF₂ | Z-4653 | —C(═O)Et | —C(═O)CH₂CH₂CH₂CH₂CH═CF₂ |
| Z-4654 | —C(═O)—c-Pr | —C(═O)C≡CH | Z-4655 | —C(═O)OMe | —C(═O)C≡CH | Z-4656 | —C(═O)Et | —C(═O)C≡CH |
| Z-4657 | —C(═O)—c-Pr | —C(═O)C≡CCH₃ | Z-4658 | —C(═O)OMe | —C(═O)C≡CCH₃ | Z-4659 | —C(═O)Et | —C(═O)C≡CCH₃ |
| Z-4660 | —C(═O)—c-Pr | —C(═O)CH₂C≡CH | Z-4661 | —C(═O)OMe | —C(═O)CH₂C≡CH | Z-4662 | —C(═O)Et | —C(═O)CH₂C≡CH |
| Z-4663 | —C(═O)—c-Pr | —C(═O)C≡CCH₂CH₃ | Z-4664 | —C(═O)OMe | —C(═O)C≡CCH₂CH₃ | Z-4665 | —C(═O)Et | —C(═O)C≡CCH₂CH₃ |
| Z-4666 | —C(═O)—c-Pr | —C(═O)CH₂C≡CCH₃ | Z-4667 | —C(═O)OMe | —C(═O)CH₂C≡CCH₃ | Z-4668 | —C(═O)Et | —C(═O)CH₂C≡CCH₃ |
| Z-4669 | —C(═O)—c-Pr | —C(═O)CH₂CH₂C≡CH | Z-4670 | —C(═O)OMe | —C(═O)CH₂CH₂C≡CH | Z-4671 | —C(═O)Et | —C(═O)CH₂CH₂C≡CH |
| Z-4672 | —C(═O)—c-Pr | —C(═O)C≡CCH₂CH₂CH₃ | Z-4673 | —C(═O)OMe | —C(═O)C≡CCH₂CH₂CH₃ | Z-4674 | —C(═O)Et | —C(═O)C≡CCH₂CH₂CH₃ |
| Z-4675 | —C(═O)—c-Pr | —C(═O)CH₂C≡CCH₂CH₃ | Z-4676 | —C(═O)OMe | —C(═O)CH₂C≡CCH₂CH₃ | Z-4677 | —C(═O)Et | —C(═O)CH₂C≡CCH₂CH₃ |
| Z-4678 | —C(═O)—c-Pr | —C(═O)C(CH₃)₂C≡CH | Z-4679 | —C(═O)OMe | —C(═O)C(CH₃)₂C≡CH | Z-4680 | —C(═O)Et | —C(═O)C(CH₃)₂C≡CH |
| Z-4681 | —C(═O)—c-Pr | —C(═O)C≡CF | Z-4682 | —C(═O)OMe | —C(═O)C≡CF | Z-4683 | —C(═O)Et | —C(═O)C≡CF |
| Z-4684 | —C(═O)—c-Pr | —C(═O)C≡CCF₂H | Z-4685 | —C(═O)OMe | —C(═O)C≡CCF₂H | Z-4686 | —C(═O)Et | —C(═O)C≡CCF₂H |
| Z-4687 | —C(═O)—c-Pr | —C(═O)C≡CCF₃ | Z-4688 | —C(═O)OMe | —C(═O)C≡CCF₃ | Z-4689 | —C(═O)Et | —C(═O)C≡CCF₃ |
| Z-4690 | —C(═O)—c-Pr | —C(═O)C≡CCH₂CF₂H | Z-4691 | —C(═O)OMe | —C(═O)C≡CCH₂CF₂H | Z-4692 | —C(═O)Et | —C(═O)C≡CCH₂CF₂H |
| Z-4693 | —C(═O)—c-Pr | —C(═O)C≡CCH₂CF₃ | Z-4694 | —C(═O)OMe | —C(═O)C≡CCH₂CF₃ | Z-4695 | —C(═O)Et | —C(═O)C≡CCH₂CF₃ |
| Z-4696 | —C(═O)—c-Pr | —C(═O)CH₂C≡CHCF₂H | Z-4697 | —C(═O)OMe | —C(═O)CH₂C≡CHCF₂H | Z-4698 | —C(═O)Et | —C(═O)CH₂C≡CHCF₂H |
| Z-4699 | —C(═O)—c-Pr | —C(═O)CH₂C≡CCF₃ | Z-4700 | —C(═O)OMe | —C(═O)CH₂C≡CCF₃ | Z-4701 | —C(═O)Et | —C(═O)CH₂C≡CCF₃ |
| Z-4702 | —C(═O)—c-Pr | —C(═O)CH₂C≡N | Z-4703 | —C(═O)OMe | —C(═O)CH₂C≡N | Z-4704 | —C(═O)Et | —C(═O)CH₂C≡N |
| Z-4705 | —C(═O)—c-Pr | —C(═O)C(Me)C≡N | Z-4706 | —C(═O)OMe | —C(═O)C(Me)C≡N | Z-4707 | —C(═O)Et | —C(═O)C(Me)C≡N |
| Z-4708 | —C(═O)—c-Pr | —C(═O)CH₂CH₂C≡N | Z-4709 | —C(═O)OMe | —C(═O)CH₂CH₂C≡N | Z-4710 | —C(═O)Et | —C(═O)CH₂CH₂C≡N |
| Z-4711 | —C(═O)—c-Pr | —C(═O)CH₂CH₂CH₂C≡N | Z-4712 | —C(═O)OMe | —C(═O)CH₂CH₂CH₂C≡N | Z-4713 | —C(═O)Et | —C(═O)CH₂CH₂CH₂C≡N |
| Z-4714 | —C(═O)—c-Pr | —C(═O)CH₂OH | Z-4715 | —C(═O)OMe | —C(═O)CH₂OH | Z-4716 | —C(═O)Et | —C(═O)CH₂OH |
| Z-4717 | —C(═O)—c-Pr | —C(═O)CH₂OMe | Z-4718 | —C(═O)OMe | —C(═O)CH₂OMe | Z-4719 | —C(═O)Et | —C(═O)CH₂OMe |
| Z-4720 | —C(═O)—c-Pr | —C(═O)CH₂OEt | Z-4721 | —C(═O)OMe | —C(═O)CH₂OEt | Z-4722 | —C(═O)Et | —C(═O)CH₂OEt |
| Z-4723 | —C(═O)—c-Pr | —C(═O)CH₂OPr | Z-4724 | —C(═O)OMe | —C(═O)CH₂OPr | Z-4725 | —C(═O)Et | —C(═O)CH₂OPr |
| Z-4726 | —C(═O)—c-Pr | —C(═O)CH₂CH₂OMe | Z-4727 | —C(═O)OMe | —C(═O)CH₂CH₂OMe | Z-4728 | —C(═O)Et | —C(═O)CH₂CH₂OMe |
| Z-4729 | —C(═O)—c-Pr | —C(═O)CH₂CH₂OEt | Z-4730 | —C(═O)OMe | —C(═O)CH₂CH₂OEt | Z-4731 | —C(═O)Et | —C(═O)CH₂CH₂OEt |
| Z-4732 | —C(═O)—c-Pr | —C(═O)CH₂—(1-Pyra) | Z-4733 | —C(═O)OMe | —C(═O)CH₂—(1-Pyra) | Z-4734 | —C(═O)Et | —C(═O)CH₂—(1-Pyra) |
| Z-4735 | —C(═O)—c-Pr | —C(═O)CH₂—(1-Tria) | Z-4736 | —C(═O)OMe | —C(═O)CH₂—(1-Tria) | Z-4737 | —C(═O)Et | —C(═O)CH₂—(1-Tria) |
| Z-4738 | —C(═O)—c-Pr | —C(═O)OH | Z-4739 | —C(═O)OMe | —C(═O)OH | Z-4740 | —C(═O)Et | —C(═O)OH |
| Z-4741 | —C(═O)—c-Pr | —C(═O)OMe | Z-4742 | —C(═O)OMe | —C(═O)OMe | Z-4743 | —C(═O)Et | —C(═O)OMe |
| Z-4744 | —C(═O)—c-Pr | —C(═O)OEt | Z-4745 | —C(═O)OMe | —C(═O)OEt | Z-4746 | —C(═O)Et | —C(═O)OEt |
| Z-4747 | —C(═O)—c-Pr | —C(═O)OPr | Z-4748 | —C(═O)OMe | —C(═O)OPr | Z-4749 | —C(═O)Et | —C(═O)OPr |
| Z-4750 | —C(═O)—c-Pr | —C(═O)O—i-Pr | Z-4751 | —C(═O)OMe | —C(═O)O—i-Pr | Z-4752 | —C(═O)Et | —C(═O)O—i-Pr |
| Z-4753 | —C(═O)—c-Pr | —C(═O)OBu | Z-4754 | —C(═O)OMe | —C(═O)OBu | Z-4755 | —C(═O)Et | —C(═O)OBu |
| Z-4756 | —C(═O)—c-Pr | —C(═O)O—sec-Bu | Z-4757 | —C(═O)OMe | —C(═O)O—sec-Bu | Z-4758 | —C(═O)Et | —C(═O)O—sec-Bu |
| Z-4759 | —C(═O)—c-Pr | —C(═O)O—i-Bu | Z-4760 | —C(═O)OMe | —C(═O)O—i-Bu | Z-4761 | —C(═O)Et | —C(═O)O—i-Bu |
| Z-4762 | —C(═O)—c-Pr | —C(═O)O—t-Bu | Z-4763 | —C(═O)OMe | —C(═O)O—t-Bu | Z-4764 | —C(═O)Et | —C(═O)O—t-Bu |
| Z-4765 | —C(═O)—c-Pr | —C(═O)OPent | Z-4766 | —C(═O)OMe | —C(═O)OPent | Z-4767 | —C(═O)Et | —C(═O)OPent |
| Z-4768 | —C(═O)—c-Pr | —C(═O)OHex | Z-4769 | —C(═O)OMe | —C(═O)OHex | Z-4770 | —C(═O)Et | —C(═O)OHex |
| Z-4771 | —C(═O)—c-Pr | —C(═O)OCH(CH₃)CH₂CH₂CH₃ | Z-4772 | —C(═O)OMe | —C(═O)OCH(CH₃)CH₂CH₂CH₃ | Z-4773 | —C(═O)Et | —C(═O)OCH(CH₃)CH₂CH₂CH₃ |
| Z-4774 | —C(═O)—c-Pr | —C(═O)OCH(CH₃)CH(CH₃)₂ | Z-4775 | —C(═O)OMe | —C(═O)OCH(CH₃)CH(CH₃)₂ | Z-4776 | —C(═O)Et | —C(═O)OCH(CH₃)CH(CH₃)₂ |
| Z-4777 | —C(═O)—c-Pr | —C(═O)OC(CH₃)₂CH₂CH₃ | Z-4778 | —C(═O)OMe | —C(═O)OC(CH₃)₂CH₂CH₃ | Z-4779 | —C(═O)Et | —C(═O)OC(CH₃)₂CH₂CH₃ |
| Z-4780 | —C(═O)—c-Pr | —C(═O)OCH(CH₂CH₃)₂ | Z-4781 | —C(═O)OMe | —C(═O)OCH(CH₂CH₃)₂ | Z-4782 | —C(═O)Et | —C(═O)OCH(CH₂CH₃)₂ |
| Z-4783 | —C(═O)—c-Pr | —C(═O)OCH₂CH₂CH(CH₃)₂ | Z-4784 | —C(═O)OMe | —C(═O)OCH₂CH₂CH(CH₃)₂ | Z-4785 | —C(═O)Et | —C(═O)OCH₂CH₂CH(CH₃)₂ |
| Z-4786 | —C(═O)—c-Pr | —C(═O)OCH₂CH₂CH₂CH(CH₃)₂ | Z-4787 | —C(═O)OMe | —C(═O)OCH₂CH₂CH₂CH(CH₃)₂ | Z-4788 | —C(═O)Et | —C(═O)OCH₂CH₂CH₂CH(CH₃)₂ |
| Z-4789 | —C(═O)—c-Pr | —C(═O)OCH₂CH₂CH(CH₃)CH₂CH₃ | Z-4790 | —C(═O)OMe | —C(═O)OCH₂CH₂CH(CH₃)CH₂CH₃ | Z-4791 | —C(═O)Et | —C(═O)OCH₂CH₂CH(CH₃)CH₂CH₃ |
| Z-4792 | —C(═O)—c-Pr | —C(═O)OCH₂CH(CH₃)CH₂CH₂CH₃ | Z-4793 | —C(═O)OMe | —C(═O)OCH₂CH(CH₃)CH₂CH₂CH₃ | Z-4794 | —C(═O)Et | —C(═O)OCH₂CH(CH₃)CH₂CH₂CH₃ |
| Z-4795 | —C(═O)—c-Pr | —C(═O)OCH(CH₃)CH₂CH₂CH₂CH₃ | Z-4796 | —C(═O)OMe | —C(═O)OCH(CH₃)CH₂CH₂CH₂CH₃ | Z-4797 | —C(═O)Et | —C(═O)OCH(CH₃)CH₂CH₂CH₂CH₃ |
| Z-4798 | —C(═O)—c-Pr | —C(═O)OCH(CH₃)CH₂CH(CH₃)₂ | Z-4799 | —C(═O)OMe | —C(═O)OCH(CH₃)CH₂CH(CH₃)₂ | Z-4800 | —C(═O)Et | —C(═O)OCH(CH₃)CH₂CH(CH₃)₂ |
| Z-4801 | —C(═O)—c-Pr | —C(═O)OCH(CH₃)CH(CH₃)CH₂CH₃ | Z-4802 | —C(═O)OMe | —C(═O)OCH(CH₃)CH(CH₃)CH₂CH₃ | Z-4803 | —C(═O)Et | —C(═O)OCH(CH₃)CH(CH₃)CH₂CH₃ |
| Z-4804 | —C(═O)—c-Pr | —C(═O)OC(CH₃)₂CH₂CH₂CH₃ | Z-4805 | —C(═O)OMe | —C(═O)OC(CH₃)₂CH₂CH₂CH₃ | Z-4806 | —C(═O)Et | —C(═O)OC(CH₃)₂CH₂CH₂CH₃ |
| Z-4807 | —C(═O)—c-Pr | —C(═O)OCH(CH₃)C(CH₃)₃ | Z-4808 | —C(═O)OMe | —C(═O)OCH(CH₃)C(CH₃)₃ | Z-4809 | —C(═O)Et | —C(═O)OCH(CH₃)C(CH₃)₃ |
| Z-4810 | —C(═O)—c-Pr | —C(═O)OC(CH₃)₂CH(CH₃)₂ | Z-4811 | —C(═O)OMe | —C(═O)OC(CH₃)₂CH(CH₃)₂ | Z-4812 | —C(═O)Et | —C(═O)OC(CH₃)₂CH(CH₃)₂ |
| Z-4813 | —C(═O)—c-Pr | —C(═O)OCH₂CH₂C(CH₃)₃ | Z-4814 | —C(═O)OMe | —C(═O)OCH₂CH₂C(CH₃)₃ | Z-4815 | —C(═O)Et | —C(═O)OCH₂CH₂C(CH₃)₃ |
| Z-4816 | —C(═O)—c-Pr | —C(═O)OCH₂CH(CH₃)CH(CH₃)₂ | Z-4817 | —C(═O)OMe | —C(═O)OCH₂CH(CH₃)CH(CH₃)₂ | Z-4818 | —C(═O)Et | —C(═O)OCH₂CH(CH₃)CH(CH₃)₂ |
| Z-4819 | —C(═O)—c-Pr | —C(═O)OCH₂C(CH₃)₂CH₂CH₃ | Z-4820 | —C(═O)OMe | —C(═O)OCH₂C(CH₃)₂CH₂CH₃ | Z-4821 | —C(═O)Et | —C(═O)OCH₂C(CH₃)₂CH₂CH₃ |
| Z-4822 | —C(═O)—c-Pr | —C(═O)OCFH₂ | Z-4823 | —C(═O)OMe | —C(═O)OCFH₂ | Z-4824 | —C(═O)Et | —C(═O)OCFH₂ |
| Z-4825 | —C(═O)—c-Pr | —C(═O)OCF₂H | Z-4826 | —C(═O)OMe | —C(═O)OCF₂H | Z-4827 | —C(═O)Et | —C(═O)OCF₂H |
| Z-4828 | —C(═O)—c-Pr | —C(═O)OCF₃ | Z-4829 | —C(═O)OMe | —C(═O)OCF₃ | Z-4830 | —C(═O)Et | —C(═O)OCF₃ |
| Z-4831 | —C(═O)—c-Pr | —C(═O)OCH₂Cl | Z-4832 | —C(═O)OMe | —C(═O)OCH₂Cl | Z-4833 | —C(═O)Et | —C(═O)OCH₂Cl |
| Z-4834 | —C(═O)—c-Pr | —C(═O)OCHCl₂ | Z-4835 | —C(═O)OMe | —C(═O)OCHCl₂ | Z-4836 | —C(═O)Et | —C(═O)OCHCl₂ |
| Z-4837 | —C(═O)—c-Pr | —C(═O)OCCl₃ | Z-4838 | —C(═O)OMe | —C(═O)OCCl₃ | Z-4839 | —C(═O)Et | —C(═O)OCCl₃ |
| Z-4840 | —C(═O)—c-Pr | —C(═O)OCH₂Br | Z-4841 | —C(═O)OMe | —C(═O)OCH₂Br | Z-4842 | —C(═O)Et | —C(═O)OCH₂Br |
| Z-4843 | —C(═O)—c-Pr | —C(═O)OCHBr₂ | Z-4844 | —C(═O)OMe | —C(═O)OCHBr₂ | Z-4845 | —C(═O)Et | —C(═O)OCHBr₂ |
| Z-4846 | —C(═O)—c-Pr | —C(═O)OCBr₃ | Z-4847 | —C(═O)OMe | —C(═O)OCBr₃ | Z-4848 | —C(═O)Et | —C(═O)OCBr₃ |
| Z-4849 | —C(═O)—c-Pr | —C(═O)OCH₂l | Z-4850 | —C(═O)OMe | —C(═O)OCH₂l | Z-4851 | —C(═O)Et | —C(═O)OCH₂l |
| Z-4852 | —C(═O)—c-Pr | —C(═O)OCHl₂ | Z-4853 | —C(═O)OMe | —C(═O)OCHl₂ | Z-4854 | —C(═O)Et | —C(═O)OCHl₂ |
| Z-4855 | —C(═O)—c-Pr | —C(═O)OCH₂CF₂H | Z-4856 | —C(═O)OMe | —C(═O)OCH₂CF₂H | Z-4857 | —C(═O)Et | —C(═O)OCH₂CF₂H |
| Z-4858 | —C(═O)—c-Pr | —C(═O)OCH₂CF₃ | Z-4859 | —C(═O)OMe | —C(═O)OCH₂CF₃ | Z-4860 | —C(═O)Et | —C(═O)OCH₂CF₃ |
| Z-4861 | —C(═O)—c-Pr | —C(═O)OCH₂CH₂CF₂H | Z-4862 | —C(═O)OMe | —C(═O)OCH₂CH₂CF₂H | Z-4863 | —C(═O)Et | —C(═O)OCH₂CH₂CF₂H |
| Z-4864 | —C(═O)—c-Pr | —C(═O)OCH₂CH₂CF₃ | Z-4865 | —C(═O)OMe | —C(═O)OCH₂CH₂CF₃ | Z-4866 | —C(═O)Et | —C(═O)OCH₂CH₂CF₃ |
| Z-4867 | —C(═O)—c-Pr | —C(═O)OCH₂CH₂CH₂CF₂H | Z-4868 | —C(═O)OMe | —C(═O)OCH₂CH₂CH₂CF₂H | Z-4869 | —C(═O)Et | —C(═O)OCH₂CH₂CH₂CF₂H |
| Z-4870 | —C(═O)—c-Pr | —C(═O)OCH₂CH₂CH₂CF₃ | Z-4871 | —C(═O)OMe | —C(═O)OCH₂CH₂CH₂CF₃ | Z-4872 | —C(═O)Et | —C(═O)OCH₂CH₂CH₂CF₃ |
| Z-4873 | —C(═O)—c-Pr | —C(═O)OCF₂CF₂H | Z-4874 | —C(═O)OMe | —C(═O)OCF₂CF₂H | Z-4875 | —C(═O)Et | —C(═O)OCF₂CF₂H |
| Z-4876 | —C(═O)—c-Pr | —C(═O)OCF₂CF₃ | Z-4877 | —C(═O)OMe | —C(═O)OCF₂CF₃ | Z-4878 | —C(═O)Et | —C(═O)OCF₂CF₃ |
| Z-4879 | —C(═O)—c-Pr | —C(═O)OCFHCF₃ | Z-4880 | —C(═O)OMe | —C(═O)OCFHCF₃ | Z-4881 | —C(═O)Et | —C(═O)OCFHCF₃ |
| Z-4882 | —C(═O)—c-Pr | —C(═O)OCH₂CF₂CF₂H | Z-4883 | —C(═O)OMe | —C(═O)OCH₂CF₂CF₂H | Z-4884 | —C(═O)Et | —C(═O)OCH₂CF₂CF₂H |
| Z-4885 | —C(═O)—c-Pr | —C(═O)OCH₂CF₂CF₃ | Z-4886 | —C(═O)OMe | —C(═O)OCH₂CF₂CF₃ | Z-4887 | —C(═O)Et | —C(═O)OCH₂CF₂CF₃ |
| Z-4888 | —C(═O)—c-Pr | —C(═O)OCF₂CF₂CF₃ | Z-4889 | —C(═O)OMe | —C(═O)OCF₂CF₂CF₃ | Z-4890 | —C(═O)Et | —C(═O)OCF₂CF₂CF₃ |
| Z-4891 | —C(═O)—c-Pr | —C(═O)OCH₂CF₂CF₂CF₃ | Z-4892 | —C(═O)OMe | —C(═O)OCH₂CF₂CF₂CF₃ | Z-4893 | —C(═O)Et | —C(═O)OCH₂CF₂CF₂CF₃ |
| Z-4894 | —C(═O)—c-Pr | —C(═O)OCF₂CF₂CF₂CF₃ | Z-4895 | —C(═O)OMe | —C(═O)OCF₂CF₂CF₂CF₃ | Z-4896 | —C(═O)Et | —C(═O)OCF₂CF₂CF₂CF₃ |
| Z-4897 | —C(═O)—c-Pr | —C(═O)OCH₂CF₂CF₂CF₂CF₃ | Z-4898 | —C(═O)OMe | —C(═O)OCH₂CF₂CF₂CF₂CF₃ | Z-4899 | —C(═O)Et | —C(═O)OCH₂CF₂CF₂CF₂CF₃ |
| Z-4900 | —C(═O)—c-Pr | —C(═O)O—c-Pr | Z-4901 | —C(═O)OMe | —C(═O)O—c-Pr | Z-4902 | —C(═O)Et | —C(═O)O—c-Pr |
| Z-4903 | —C(═O)—c-Pr | —C(═O)O—c-Bu | Z-4904 | —C(═O)OMe | —C(═O)O—c-Bu | Z-4905 | —C(═O)Et | —C(═O)O—c-Bu |
| Z-4906 | —C(═O)—c-Pr | —C(═O)O—c-Pent | Z-4907 | —C(═O)OMe | —C(═O)O—c-Pent | Z-4908 | —C(═O)Et | —C(═O)O—c-Pent |
| Z-4909 | —C(═O)—c-Pr | —C(═O)O—c-Hex | Z-4910 | —C(═O)OMe | —C(═O)O—c-Hex | Z-4911 | —C(═O)Et | —C(═O)O—c-Hex |
| Z-4912 | —C(═O)—c-Pr | —C(═O)O—c-Hept | Z-4913 | —C(═O)OMe | —C(═O)O—c-Hept | Z-4914 | —C(═O)Et | —C(═O)O—c-Hept |
| Z-4915 | —C(═O)—c-Pr | —C(═O)O—c-Oct | Z-4916 | —C(═O)OMe | —C(═O)O—c-Oct | Z-4917 | —C(═O)Et | —C(═O)O—c-Oct |
| Z-4918 | —C(═O)—c-Pr | —C(═O)OCH═CH₂ | Z-4919 | —C(═O)OMe | —C(═O)OCH═CH₂ | Z-4920 | —C(═O)Et | —C(═O)OCH═CH₂ |
| Z-4921 | —C(═O)—c-Pr | —C(═O)OCH₂CH═CH₂ | Z-4922 | —C(═O)OMe | —C(═O)OCH₂CH═CH₂ | Z-4923 | —C(═O)Et | —C(═O)OCH₂CH═CH₂ |
| Z-4924 | —C(═O)—c-Pr | —C(═O)OCH═CHCH₃ | Z-4925 | —C(═O)OMe | —C(═O)OCH═CHCH₃ | Z-4926 | —C(═O)Et | —C(═O)OCH═CHCH₃ |
| Z-4927 | —C(═O)—c-Pr | —C(═O)OCH₂C(CH₃)═CH₂ | Z-4928 | —C(═O)OMe | —C(═O)OCH₂C(CH₃)═CH₂ | Z-4929 | —C(═O)Et | —C(═O)OCH₂C(CH₃)═CH₂ |
| Z-4930 | —C(═O)—c-Pr | —C(═O)OCH₂CH₂CH═CH₂ | Z-4931 | —C(═O)OMe | —C(═O)OCH₂CH₂CH═CH₂ | Z-4932 | —C(═O)Et | —C(═O)OCH₂CH₂CH═CH₂ |
| Z-4933 | —C(═O)—c-Pr | —C(═O)OCH₂CH═CHCH₃ | Z-4934 | —C(═O)OMe | —C(═O)OCH₂CH═CHCH₃ | Z-4935 | —C(═O)Et | —C(═O)OCH₂CH═CHCH₃ |
| Z-4936 | —C(═O)—c-Pr | —C(═O)OCH═CHCH₂CH₃ | Z-4937 | —C(═O)OMe | —C(═O)OCH═CHCH₂CH₃ | Z-4938 | —C(═O)Et | —C(═O)OCH═CHCH₂CH₃ |
| Z-4939 | —C(═O)—c-Pr | —C(═O)OCH₂CH═C(CH₃)₂ | Z-4940 | —C(═O)OMe | —C(═O)OCH₂CH═C(CH₃)₂ | Z-4941 | —C(═O)Et | —C(═O)OCH₂CH═C(CH₃)₂ |
| Z-4942 | —C(═O)—c-Pr | —C(═O)OCH2CH₂CH═C(CH₃)₂ | Z-4943 | —C(═O)OMe | —C(═O)OCH2CH₂CH═C(CH₃)₂ | Z-4944 | —C(═O)Et | —C(═O)OCH2CH₂CH═C(CH₃)₂ |
| Z-4945 | —C(═O)—c-Pr | —C(═O)OCH═CFH | Z-4946 | —C(═O)OMe | —C(═O)OCH═CFH | Z-4947 | —C(═O)Et | —C(═O)OCH═CFH |
| Z-4948 | —C(═O)—c-Pr | —C(═O)OCH═CF₂ | Z-4949 | —C(═O)OMe | —C(═O)OCH═CF₂ | Z-4950 | —C(═O)Et | —C(═O)OCH═CF₂ |
| Z-4951 | —C(═O)—c-Pr | —C(═O)OCH═CCl₂ | Z-4952 | —C(═O)OMe | —C(═O)OCH═CCl₂ | Z-4953 | —C(═O)Et | —C(═O)OCH═CCl₂ |
| Z-4954 | —C(═O)—c-Pr | —C(═O)OCH₂CH═CFH | Z-4955 | —C(═O)OMe | —C(═O)OCH₂CH═CFH | Z-4956 | —C(═O)Et | —C(═O)OCH₂CH═CFH |
| Z-4957 | —C(═O)—c-Pr | —C(═O)OCH₂CH═CF₂ | Z-4958 | —C(═O)OMe | —C(═O)OCH₂CH═CF₂ | Z-4959 | —C(═O)Et | —C(═O)OCH₂CH═CF₂ |
| Z-4960 | —C(═O)—c-Pr | —C(═O)OCH₂CH═CCl₂ | Z-4961 | —C(═O)OMe | —C(═O)OCH₂CH═CCl₂ | Z-4962 | —C(═O)Et | —C(═O)OCH₂CH═CCl₂ |
| Z-4963 | —C(═O)—c-Pr | —C(═O)OCH₂CH₂CH═CF₂ | Z-4964 | —C(═O)OMe | —C(═O)OCH₂CH₂CH═CF₂ | Z-4965 | —C(═O)Et | —C(═O)OCH₂CH₂CH═CF₂ |
| Z-4966 | —C(═O)—c-Pr | —C(═O)OCH₂CH₂CH₂CH═CF₂ | Z-4967 | —C(═O)OMe | —C(═O)OCH₂CH₂CH₂CH═CF₂ | Z-4968 | —C(═O)Et | —C(═O)OCH₂CH₂CH₂CH═CF₂ |
| Z-4969 | —C(═O)—c-Pr | —C(═O)OCH₂CH₂CH₂CH₂CH═CF₂ | Z-4970 | —C(═O)OMe | —C(═O)OCH₂CH₂CH₂CH₂CH═CF₂ | Z-4971 | —C(═O)Et | —C(═O)OCH₂CH₂CH₂CH₂CH═CF₂ |
| Z-4972 | —C(═O)—c-Pr | —C(═Q)OCH₂C≡CH | Z-4973 | —C(═O)OMe | —C(═Q)OCH₂C≡CH | Z-4974 | —C(═O)Et | —C(═Q)OCH₂C≡CH |
| Z-4975 | —C(═O)—c-Pr | —C(═O)OCH₂C≡CCH₃ | Z-4976 | —C(═O)OMe | —C(═O)OCH₂C≡CCH₃ | Z-4977 | —C(═O)Et | —C(═O)OCH₂C≡CCH₃ |
| Z-4978 | —C(═O)—c-Pr | —C(═O)OCH₂CH₂C≡CH | Z-4979 | —C(═O)OMe | —C(═O)OCH₂CH₂C≡CH | Z-4980 | —C(═O)Et | —C(═O)OCH₂CH₂C≡CH |
| Z-4981 | —C(═O)—c-Pr | —C(═O)OCH₂C≡CCH₂CH₃ | Z-4982 | —C(═O)OMe | —C(═O)OCH₂C≡CCH₂CH₃ | Z-4983 | —C(═O)Et | —C(═O)OCH₂C≡CCH₂CH₃ |
| Z-4984 | —C(═O)—c-Pr | —C(═O)OC(CH₃)₂C≡CH | Z-4985 | —C(═O)OMe | —C(═O)OC(CH₃)₂C≡CH | Z-4986 | —C(═O)Et | —C(═O)OC(CH₃)₂C≡CH |
| Z-4987 | —C(═O)—c-Pr | —C(═O)OCH₂C≡CHCF₂H | Z-4988 | —C(═O)OMe | —C(═O)OCH₂C≡CHCF₂H | Z-4989 | —C(═O)Et | —C(═O)OCH₂C≡CHCF₂H |
| Z-4990 | —C(═O)—c-Pr | —C(═O)OCH₂C≡CCF₃ | Z-4991 | —C(═O)OMe | —C(═O)OCH₂C≡CCF₃ | Z-4992 | —C(═O)Et | —C(═O)OCH₂C≡CCF₃ |
| Z-4993 | —C(═O)—c-Pr | —C(═O)Ph | 2-4994 | —C(═O)OMe | —C(═O)Ph | Z-4995 | —C(═O)Et | —C(═O)Ph |
| Z-4996 | —C(═O)—c-Pr | —C(═O)(2-Py) | Z-4997 | —C(═O)OMe | —C(═O)(2-Py) | Z-4998 | —C(═O)Et | —C(═O)(2-Py) |
| Z-4999 | —C(═O)—c-Pr | —C(═O)(3-Py) | Z-5000 | —C(═O)OMe | —C(═O)(3-Py) | Z-5001 | —C(═O)Et | —C(═O)(3-Py) |
| Z-5002 | —C(═O)—c-Pr | —C(═O)(4-Py) | Z-5003 | —C(═O)OMe | —C(═O)(4-Py) | Z-5004 | —C(═O)Et | —C(═O)(4-Py) |
| Z-5005 | —C(═O)—c-Pr | —C(═O)CF₂Me | Z-5006 | —C(═O)OMe | —C(═O)CF₂Me | Z-5007 | —C(═O)Et | —C(═O)CF₂Me |
| Z-5008 | —C(═O)—c-Pr | —C(═O)NMe2 | Z-5009 | —C(═O)OMe | —C(═O)NMe2 | Z-5010 | —C(═O)Et | —C(═O)NMe2 |
| Z-5011 | —C(═O)—c-Pr | —C(═O)—(1-CF₃—c-Pr) | Z-5012 | —C(═O)OMe | —C(═O)—(1-CF₃—c-Pr) | Z-5013 | —C(═O)Et | —C(═O)—(1-CF₃—c-Pr) |
| Z-5014 | —C(═O)—c-Pr | —C(═O)—(1-F—c-Pr) | Z-5015 | —C(═O)OMe | —C(═O)—(1-F—c-Pr) | Z-5016 | —C(═O)Et | —C(═O)—(1-F—c-Pr) |
| Z-5017 | —C(═O)—c-Pr | —SO₂Me | Z-5018 | —C(═O)OMe | —SO₂Me | Z-5019 | —C(═O)Et | —SO₂Me |
| Z-5020 | —C(═O)—c-Pr | —C(═O)CF═CH₂ | Z-5021 | —C(═O)OMe | —C(═O)CF═CH₂ | Z-5022 | —C(═O)Et | —C(═O)CF═CH₂ |
| Z-5023 | —C(═O)—c-Pr | —C(═O)—(4-Cl—Ph) | Z-5024 | —C(═O)OMe | —C(═O)—(4-Cl—Ph) | Z-5025 | —C(═O)Et | —C(═O)—(4-Cl—Ph) |
| Z-5026 | —C(═O)—c-Pr | —C(═O)—(3-Cl—Ph) | Z-5027 | —C(═O)OMe | —C(═O)—(3-Cl—Ph) | Z-5028 | —C(═O)Et | —C(═O)—(3-Cl—Ph) |
| Z-5029 | —C(═O)—c-Pr | —C(═O)—(3-CF₃—Ph) | Z-5030 | —C(═O)OMe | —C(═O)—(3-CF₃—Ph) | Z-5031 | —C(═O)Et | —C(═O)—(3-CF₃—Ph) |
| Z-5032 | —C(═O)—c-Pr | —C(═O)—(2-Cl—Ph) | Z-5033 | —C(═O)OMe | —C(═O)—(2-Cl—Ph) | Z-5034 | —C(═O)Et | —C(═O)—(2-Cl—Ph) |
| Z-5035 | —C(═O)—c-Pr | —C(═O)—(2-CF₃—Ph) | Z-5036 | —C(═O)OMe | —C(═O)—(2-CF₃—Ph) | Z-5037 | —C(═O)Et | —C(═O)—(2-CF₃—Ph) |
| Z-5038 | —C(═O)—c-Pr | —C(═O)—(4-CF₃—Ph) | Z-5039 | —C(═O)OMe | —C(═O)—(4-CF₃—Ph) | Z-5040 | —C(═O)Et | —C(═O)—(4-CF₃—Ph) |
| Z-5041 | —C(═O)—c-Pr | —C(═O)—(3-F—Ph) | Z-5042 | —C(═O)OMe | —C(═O)—(3-F—Ph) | Z-5043 | —C(═O)Et | —C(═O)—(3-F—Ph) |
| Z-5044 | —C(═O)—c-Pr | —C(═O)—(4-F—Ph) | Z-5045 | —C(═O)OMe | —C(═O)—(4-F—Ph) | Z-5046 | —C(═O)Et | —C(═O)—(4-F—Ph) |
| Z-5047 | —C(═O)—c-Pr | —C(═O)—(2-F—Ph) | Z-5048 | —C(═O)OMe | —C(═O)—(2-F—Ph) | Z-5049 | —C(═O)Et | —C(═O)—(2-F—Ph) |
| Z-5050 | —C(═O)—c-Pr | —C(═O)—(4-OCF₃—Ph) | Z-5051 | —C(═O)OMe | —C(═O)—(4-OCF₃—Ph) | Z-5052 | —C(═O)Et | —C(═O)—(4-OCF₃—Ph) |
| Z-5053 | —C(═O)—c-Pr | —C(═O)—(6-Cl—3-Py) | Z-5054 | —C(═O)OMe | —C(═O)—(6-Cl—3-Py) | Z-5055 | —C(═O)Et | —C(═O)—(6-Cl—3-Py) |
| Z-5056 | —C(═O)—c-Pr | —C(═O)—(6-CF3—2-Py) | Z-5057 | —C(═O)OMe | —C(═O)—(6-CF3—2-Py) | Z-5058 | —C(═O)Et | —C(═O)—(6-CF3—2-Py) |
| Z-5059 | —C(═O)—c-Pr | —C(═O)—(1-CN—c-Pr) | Z-5060 | —C(═O)OMe | —C(═O)—(1-CN—c-Pr) | Z-5061 | —C(═O)Et | —C(═O)—(1-CN—c-Pr) |
| Z-5062 | —C(═O)—c-Pr | —C(═O)—(3-Cl—2-Py) | Z-5063 | —C(═O)OMe | —C(═O)—(3-Cl—2-Py) | Z-5064 | —C(═O)Et | —C(═O)—(3-Cl—2-Py) |
| Z-5065 | —C(═O)—c-Pr | —C(═O)—(2-Me—Ph) | Z-5066 | —C(═O)OMe | —C(═O)—(2-Me—Ph) | Z-5067 | —C(═O)Et | —C(═O)—(2-Me—Ph) |
| Z-5068 | —C(═O)—c-Pr | —C(═O)—(3-Me—Ph) | Z-5069 | —C(═O)OMe | —C(═O)—(3-Me—Ph) | Z-5070 | —C(═O)Et | —C(═O)—(3-Me—Ph) |
| Z-5071 | —C(═O)—c-Pr | —C(═O)—(4-Me—Ph) | Z-5072 | —C(═O)OMe | —C(═O)—(4-Me—Ph) | Z-5073 | —C(═O)Et | —C(═O)—(4-Me—Ph) |
| Z-5074 | —C(═O)—c-Pr | —C(═O)—(2-MeO—Ph) | Z-5075 | —C(═O)OMe | —C(═O)—(2-MeO—Ph) | Z-5076 | —C(═O)Et | —C(═O)—(2-MeO—Ph) |
| Z-5077 | —C(═O)—c-Pr | —C(═O)—(3-MeO—Ph) | Z-5078 | —C(═O)OMe | —C(═O)—(3-MeO—Ph) | Z-5079 | —C(═O)Et | —C(═O)—(3-MeO—Ph) |
| Z-5080 | —C(═O)—c-Pr | —C(═O)—(4-MeO—Ph) | Z-5081 | —C(═O)OMe | —C(═O)—(4-MeO—Ph) | Z-5082 | —C(═O)Et | —C(═O)—(4-MeO—Ph) |
| Z-5083 | —C(═O)—c-Pr | —C(═O)—(4-Cl—2-Py) | Z-5084 | —C(═O)OMe | —C(═O)—(4-Cl—2-Py) | Z-5085 | —C(═O)Et | —C(═O)—(4-Cl—2-Py) |
| Z-5086 | —C(═O)—c-Pr | —C(═O)—(5-Cl—2-Py) | Z-5087 | —C(═O)OMe | —C(═O)—(5-Cl—2-Py) | Z-5088 | —C(═O)Et | —C(═O)—(5-Cl—2-Py) |
| Z-5089 | —C(═O)—c-Pr | —C(═O)—(6-Cl—2-Py) | Z-5090 | —C(═O)OMe | —C(═O)—(6-Cl—2-Py) | Z-5091 | —C(═O)Et | —C(═O)—(6-Cl—2-Py) |
| Z-5092 | —C(═O)—c-Pr | —C(═O)—(2-Cl—3-Py) | Z-5093 | —C(═O)OMe | —C(═O)—(2-Cl—3-Py) | Z-5094 | —C(═O)Et | —C(═O)—(2-Cl—3-Py) |
| Z-5095 | —C(═O)—c-Pr | —C(═O)—(2-Cl—4-Py) | Z-5096 | —C(═O)OMe | —C(═O)—(2-Cl—4-Py) | Z-5097 | —C(═O)Et | —C(═O)—(2-Cl—4-Py) |
| Z-5098 | —C(═O)—c-Pr | —C(═O)—(3-Cl—4-Py) | Z-5099 | —C(═O)OMe | —C(═O)—(3-Cl—4-Py) | Z-5100 | —C(═O)Et | —C(═O)—(3-Cl—4-Py) |
| Z-5101 | —C(═O)—c-Pr | —C(═O)—(3,4-di-Me—Ph) | Z-5102 | —C(═O)OMe | —C(═O)—(3,4-di-Me—Ph) | Z-5103 | —C(═O)Et | —C(═O)—(3,4-di-Me—Ph) |
| Z-5104 | —C(═O)—c-Pr | —C(═O)—(3,5-di-Me—Ph) | Z-5105 | —C(═O)OMe | —C(═O)—(3,5-di-Me—Ph) | Z-5106 | —C(═O)Et | —C(═O)—(3,5-di-Me—Ph) |
| Z-5107 | —C(═O)—c-Pr | —C(═O)—(4-Cl—3-Py) | Z-5108 | —C(═O)OMe | —C(═O)—(4-Cl—3-Py) | Z-5109 | —C(═O)Et | —C(═O)—(4-Cl—3-Py) |
| Z-5110 | —C(═O)—c-Pr | —C(═O)—(5-Cl—3-Py) | Z-5111 | —C(═O)OMe | —C(═O)—(5-Cl—3-Py) | Z-5112 | —C(═O)Et | —C(═O)—(5-Cl—3-Py) |
| Z-5113 | —C(═O)—c-Pr | —C(═O)—(4-Pyrimidine) | Z-5114 | —C(═O)OMe | —C(═O)—(4-Pyrimidine) | Z-5115 | —C(═O)Et | —C(═O)—(4-Pyrimidine) |
| Z-5116 | —C(═O)—c-Pr | —C(═O)—(2-Cl—4-Pyrimidine) | Z-5117 | —C(═O)OMe | —C(═O)—(2-Cl—4-Pyrimidine) | Z-5118 | —C(═O)Et | —C(═O)—(2-Cl—4-Pyrimidine) |
| Z-5119 | —C(═O)—c-Pr | —C(═O)—(4-Et—Ph) | Z-5120 | —C(═O)OMe | —C(═O)—(4-Et—Ph) | Z-5121 | —C(═O)Et | —C(═O)—(4-Et—Ph) |
| Z-5122 | —C(═O)—c-Pr | —C(═O)—(2-Me—4-Pyrimidine) | Z-5123 | —C(═O)OMe | —C(═O)—(2-Me—4-Pyrimidine) | Z-5124 | —C(═O)Et | —C(═O)—(2-Me—4-Pyrimidine) |
| Z-5125 | —C(═O)—c-Pr | —C(═O)—(6-Me—4-Pyrimidine) | Z-5126 | —C(═O)OMe | —C(═O)—(6-Me—4-Pyrimidine) | Z-5127 | —C(═O)Et | —C(═O)—(6-Me—4-Pyrimidine) |
| Z-5128 | —C(═O)—c-Pr | —C(═O)—(6-Me—2-Py) | Z-5129 | —C(═O)OMe | —C(═O)—(6-Me—2-Py) | Z-5130 | —C(═O)Et | —C(═O)—(6-Me—2-Py) |
| Z-5131 | —C(═O)—c-Pr | —C(═O)—(2-CF₃—4-Pyrimidine) | Z-5132 | —C(═O)OMe | —C(═O)—(2-CF₃—4-Pyrimidine) | Z-5133 | —C(═O)Et | —C(═O)—(2-CF₃—4-Pyrimidine) |
| Z-5134 | —C(═O)—c-Pr | —C(═O)—(3-Pyridazine) | Z-5135 | —C(═O)OMe | —C(═O)—(3-Pyridazine) | Z-5136 | —C(═O)Et | —C(═O)—(3-Pyridazine) |
| Z-5137 | —C(═O)—c-Pr | —C(═O)—(1-Me—c-Pr) | Z-5138 | —C(═O)OMe | —C(═O)—(1-Me—c-Pr) | Z-5139 | —C(═O)Et | —C(═O)—(1-Me—c-Pr) |
| Z-5140 | —C(═O)—c-Pr | —C(═O)—(1-CF₃—c-Bu) | Z-5141 | —C(═O)OMe | —C(═O)—(1-CF₃—c-Bu) | Z-5142 | —C(═O)Et | —C(═O)—(1-CF₃—c-Bu) |
| Z-5143 | —C(═O)—c-Pr | —C(═O)—(2-Pyrimidine) | Z-5144 | —C(═O)OMe | —C(═O)—(2-Pyrimidine) | Z-5145 | —C(═O)Et | —C(═O)—(2-Pyrimidine) |
| Z-5146 | —C(═O)—c-Pr | —C(═O)—(2-Pyrazine) | Z-5147 | —C(═O)OMe | —C(═O)—(2-Pyrazine) | Z-5148 | —C(═O)Et | —C(═O)—(2-Pyrazine) |
| Z-5149 | —C(═O)—c-Pr | —C(═O)CH═CHOEt | Z-5150 | —C(═O)OMe | —C(═O)CH═CHOEt | Z-5151 | —C(═O)Et | —C(═O)CH═CHOEt |
| Z-5152 | —C(═O)—c-Pr | —C(═O)CH₂CHCF₃CF₃ | Z-5153 | —C(═O)OMe | —C(═O)CH₂CHCF₃CF₃ | Z-5154 | —C(═O)Et | —C(═O)CH₂CHCF₃CF₃ |
| Z-5155 | —C(═O)—c-Pr | —C(═O)CH₂—(c-Pr) | Z-5156 | —C(═O)OMe | —C(═O)CH₂—(c-Pr) | Z-5157 | —C(═O)Et | —C(═O)CH₂—(c-Pr) |
| Z-5158 | —C(═O)—c-Pr | —C(═O)—(2,2,3,3-tetra-Me—c-Pr) | Z-5159 | —C(═O)OMe | —C(═O)—(2,2,3,3-tetra-Me—c-Pr) | Z-5160 | —C(═O)Et | —C(═O)—(2,2,3,3-tetra-Me—c-Pr) |
| Z-5161 | —C(═O)—c-Pr | —C(═O)CMe₂CF₃ | Z-5162 | —C(═O)OMe | —C(═O)CMe₂CF₃ | Z-5163 | —C(═O)Et | —C(═O)CMe₂CF₃ |
| Z-5164 | —C(═O)—c-Hex | —H | Z-5165 | —C(═O)—Ph | —H | Z-5166 | —C(═O)—4-OCF₃—Ph | —H |
| Z-5167 | —C(═O)—c-Hex | —Me | Z-5168 | —C(═O)—Ph | —Me | Z-5169 | —C(═O)—4-OCF₃—Ph | —Me |
| Z-5170 | —C(═O)—c-Hex | —Et | Z-5171 | —C(═O)—Ph | —Et | Z-5172 | —C(═O)—4-OCF₃—Ph | —Et |
| Z-5173 | —C(═O)—c-Hex | —Pr | Z-5174 | —C(═O)—Ph | —Pr | Z-5175 | —C(═O)—4-OCF₃—Ph | —Pr |
| Z-5176 | —C(═O)—c-Hex | —i-Pr | Z-5177 | —C(═O)—Ph | —i-Pr | Z-5178 | —C(═O)—4-OCF₃—Ph | —i-Pr |
| Z-5179 | —C(═O)—c-Hex | —Bu | Z-5180 | —C(═O)—Ph | —Bu | Z-5181 | —C(═O)—4-OCF₃—Ph | —Bu |
| Z-5182 | —C(═O)—c-Hex | —sec-Bu | Z-5183 | —C(═O)—Ph | —sec-Bu | Z-5184 | —C(═O)—4-OCF₃—Ph | —sec-Bu |
| Z-5185 | —C(═O)—c-Hex | —I-Bu | Z-5186 | —C(═O)—Ph | —I-Bu | Z-5187 | —C(═O)—4-OCF₃—Ph | —I-Bu |
| Z-5188 | —C(═O)—c-Hex | —t-Bu | Z-5189 | —C(═O)—Ph | —t-Bu | Z-5190 | —C(═O)—4-OCF₃—Ph | —t-Bu |
| Z-5191 | —C(═O)—c-Hex | —Pent | Z-5192 | —C(═O)—Ph | —Pent | Z-5193 | —C(═O)—4-OCF₃—Ph | —Pent |
| Z-5194 | —C(═O)—c-Hex | —Hex | Z-5195 | —C(═O)—Ph | —Hex | Z-5196 | —C(═O)—4-OCF₃—Ph | —Hex |
| Z-5197 | —C(═O)—c-Hex | —CH(CH₃)CH₂CH₂CH₃ | Z-5198 | —C(═O)—Ph | —CH(CH₃)CH₂CH₂CH₃ | Z-5199 | —C(═O)—4-OCF₃—Ph | —CH(CH₃)CH₂CH₂CH₃ |
| Z-5200 | —C(═O)—c-Hex | —CH(CH₃)CH(CH₃)₂ | Z-5201 | —C(═O)—Ph | —CH(CH₃)CH(CH₃)₂ | Z-5202 | —C(═O)—4-OCF₃—Ph | —CH(CH₃)CH(CH₃)₂ |
| Z-5203 | —C(═O)—c-Hex | —C(CH₃)₂CH₂CH₃ | Z-5204 | —C(═O)—Ph | —C(CH₃)₂CH₂CH₃ | Z-5205 | —C(═O)—4-OCF₃—Ph | —C(CH₃)₂CH₂CH₃ |
| Z-5206 | —C(═O)—c-Hex | —CH(CH₂CH₃)₂ | Z-5207 | —C(═O)—Ph | —CH(CH₂CH₃)₂ | Z-5208 | —C(═O)—4-OCF₃—Ph | —CH(CH₂CH₃)₂ |
| Z-5209 | —C(═O)—c-Hex | —CH₂CH₂CH(CH₃)₂ | Z-5210 | —C(═O)—Ph | —CH₂CH₂CH(CH₃)₂ | Z-5211 | —C(═O)—4-OCF₃—Ph | —CH₂CH₂CH(CH₃)₂ |
| Z-5212 | —C(═O)—c-Hex | —CH₂CH₂CH₂CH(CH₃)₂ | Z-5213 | —C(═O)—Ph | —CH₂CH₂CH₂CH(CH₃)₂ | Z-5214 | —C(═O)—4-OCF₃—Ph | —CH₂CH₂CH₂CH(CH₃)₂ |
| Z-5215 | —C(═O)—c-Hex | —CH₂CH₂CH(CH₃)CH₂CH₃ | Z-5216 | —C(═O)—Ph | —CH₂CH₂CH(CH₃)CH₂CH₃ | Z-5217 | —C(═O)—4-OCF₃—Ph | —CH₂CH₂CH(CH₃)CH₂CH₃ |
| Z-5218 | —C(═O)—c-Hex | —CH₂CH(CH₃)CH₂CH₂CH₃ | Z-5219 | —C(═O)—Ph | —CH₂CH(CH₃)CH₂CH₂CH₃ | Z-5220 | —C(═O)—4-OCF₃—Ph | —CH₂CH(CH₃)CH₂CH₂CH₃ |
| Z-5221 | —C(═O)—c-Hex | —CH(CH₃)CH₂CH₂CH₂CH₃ | Z-5222 | —C(═O)—Ph | —CH(CH₃)CH₂CH₂CH₂CH₃ | Z-5223 | —C(═O)—4-OCF₃—Ph | —CH(CH₃)CH₂CH₂CH₂CH₃ |
| Z-5224 | —C(═O)—c-Hex | —CH(CH3)CH₂CH(CH₃)₂ | Z-5225 | —C(═O)—Ph | —CH(CH3)CH₂CH(CH₃)₂ | Z-5226 | —C(═O)—4-OCF₃—Ph | —CH(CH3)CH₂CH(CH₃)₂ |
| Z-5227 | —C(═O)—c-Hex | —CH(CH₃)CH(CH₃)CH₂CH₃ | Z-5228 | —C(═O)—Ph | —CH(CH₃)CH(CH₃)CH₂CH₃ | Z-5229 | —C(═O)—4-OCF₃—Ph | —CH(CH₃)CH(CH₃)CH₂CH₃ |
| Z-5230 | —C(═O)—c-Hex | —C(CH₃)₂CH₂CH₂CH₃ | Z-5231 | —C(═O)—Ph | —C(CH₃)₂CH₂CH₂CH₃ | Z-5232 | —C(═O)—4-OCF₃—Ph | —C(CH₃)₂CH₂CH₂CH₃ |
| Z-5233 | —C(═O)—c-Hex | —CH(CH₃)C(CH₃)₃ | Z-5234 | —C(═O)—Ph | —CH(CH₃)C(CH₃)₃ | Z-5235 | —C(═O)—4-OCF₃—Ph | —CH(CH₃)C(CH₃)₃ |
| Z-5236 | —C(═O)—c-Hex | —C(CH₃)₂CH(CH₃)₂ | Z-5237 | —C(═O)—Ph | —C(CH₃)₂CH(CH₃)₂ | Z-5238 | —C(═O)—4-OCF₃—Ph | —C(CH₃)₂CH(CH₃)₂ |
| Z-5239 | —C(═O)—c-Hex | —CH₂CH₂C(CH₃)₃ | Z-5240 | —C(═O)—Ph | —CH₂CH₂C(CH₃)₃ | Z-5241 | —C(═O)—4-OCF₃—Ph | —CH₂CH₂C(CH₃)₃ |
| Z-5242 | —C(═O)—c-Hex | —CH₂CH(CH₃)CH(CH₃)₂ | Z-5243 | —C(═O)—Ph | —CH₂CH(CH₃)CH(CH₃)₂ | Z-5244 | —C(═O)—4-OCF₃—Ph | —CH₂CH(CH₃)CH(CH₃)₂ |
| Z-5245 | —C(═O)—c-Hex | —CH₂C(CH₃)₂CH₂CH₃ | Z-5246 | —C(═O)—Ph | —CH₂C(CH₃)₂CH₂CH₃ | Z-5247 | —C(═O)—4-OCF₃—Ph | —CH₂C(CH₃)₂CH₂CH₃ |
| Z-5248 | —C(═O)—c-Hex | —CFH₂ | Z-5249 | —C(═O)—Ph | —CFH₂ | Z-5250 | —C(═O)—4-OCF₃—Ph | —CFH₂ |
| Z-5251 | —C(═O)—c-Hex | —CF₂H | Z-5252 | —C(═O)—Ph | —CF₂H | Z-5253 | —C(═O)—4-OCF₃—Ph | —CF₂H |
| Z-5254 | —C(═O)—c-Hex | —CF₃ | Z-5255 | —C(═O)—Ph | —CF₃ | Z-5256 | —C(═O)—4-OCF₃—Ph | —CF₃ |
| Z-5257 | —C(═O)—c-Hex | —CH₂Cl | Z-5258 | —C(═O)—Ph | —CH₂Cl | Z-5259 | —C(═O)—4-OCF₃—Ph | —CH₂Cl |
| Z-5260 | —C(═O)—c-Hex | —CHCl₂ | Z-5261 | —C(═O)—Ph | —CHCl₂ | Z-5262 | —C(═O)—4-OCF₃—Ph | —CHCl₂ |
| Z-5263 | —C(═O)—c-Hex | —CCl₃ | Z-5264 | —C(═O)—Ph | —CCl₃ | Z-5265 | —C(═O)—4-OCF₃—Ph | —CCl₃ |
| Z-5266 | —C(═O)—c-Hex | —CF₂Cl | Z-5267 | —C(═O)—Ph | —CF₂Cl | Z-5268 | —C(═O)—4-OCF₃—Ph | —CF₂Cl |
| Z-5269 | —C(═O)—c-Hex | —CCl₂F | Z-5270 | —C(═O)—Ph | —CCl₂F | Z-5271 | —C(═O)—4-OCF₃—Ph | —CCl₂F |
| Z-5272 | —C(═O)—c-Hex | —CH₂Br | Z-5273 | —C(═O)—Ph | —CH₂Br | Z-5274 | —C(═O)—4-OCF₃—Ph | —CH₂Br |
| Z-5275 | —C(═O)—c-Hex | —CHBr₂ | Z-5276 | —C(═O)—Ph | —CHBr₂ | Z-5277 | —C(═O)—4-OCF₃—Ph | —CHBr₂ |
| Z-5278 | —C(═O)—c-Hex | —CBr₃ | Z-5279 | —C(═O)—Ph | —CBr₃ | Z-5280 | —C(═O)—4-OCF₃—Ph | —CBr₃ |
| Z-5281 | —C(═O)—c-Hex | —CH₂l | Z-5282 | —C(═O)—Ph | —CH₂l | Z-5283 | —C(═O)—4-OCF₃—Ph | —CH₂l |
| Z-5284 | —C(═O)—c-Hex | —CHl₂ | Z-5285 | —C(═O)—Ph | —CHl₂ | Z-5286 | —C(═O)—4-OCF₃—Ph | —CHl₂ |
| Z-5287 | —C(═O)—c-Hex | —CH₂CF₂H | Z-5288 | —C(═O)—Ph | —CH₂CF₂H | Z-5289 | —C(═O)—4-OCF₃—Ph | —CH₂CF₂H |
| Z-5290 | —C(═O)—c-Hex | —CH₂CF₃ | Z-5291 | —C(═O)—Ph | —CH₂CF₃ | Z-5292 | —C(═O)—4-OCF₃—Ph | —CH₂CF₃ |
| Z-5293 | —C(═O)—c-Hex | —CF₂CH₃ | Z-5294 | —C(═O)—Ph | —CF₂CH₃ | Z-5295 | —C(═O)—4-OCF₃—Ph | —CF₂CH₃ |
| Z-5296 | —C(═O)—c-Hex | —CH₂CH₂CF₂H | Z-5297 | —C(═O)—Ph | —CH₂CH₂CF₂H | Z-5298 | —C(═O)—4-OCF₃—Ph | —CH₂CH₂CF₂H |
| Z-5299 | —C(═O)—c-Hex | —CH₂CH₂CF₃ | Z-5300 | —C(═O)—Ph | —CH₂CH₂CF₃ | Z-5301 | —C(═O)—4-OCF₃—Ph | —CH₂CH₂CF₃ |
| Z-5302 | —C(═O)—c-Hex | —CH₂CH₂CH₂CF₂H | Z-5303 | —C(═O)—Ph | —CH₂CH₂CH₂CF₂H | Z-5304 | —C(═O)—4-OCF₃—Ph | —CH₂CH₂CH₂CF₂H |
| Z-5305 | —C(═O)—c-Hex | —CH₂CH₂CH₂CF₃ | Z-5306 | —C(═O)—Ph | —CH₂CH₂CH₂CF₃ | Z-5307 | —C(═O)—4-OCF₃—Ph | —CH₂CH₂CH₂CF₃ |
| Z-5308 | —C(═O)—c-Hex | —CF₂CF₂H | Z-5309 | —C(═O)—Ph | —CF₂CF₂H | Z-5310 | —C(═O)—4-OCF₃—Ph | —CF₂CF₂H |
| Z-5311 | —C(═O)—c-Hex | —CF₂CF₂Cl | Z-5312 | —C(═O)—Ph | —CF₂CF₂Cl | Z-5313 | —C(═O)—4-OCF₃—Ph | —CF₂CF₂Cl |
| Z-5314 | —C(═O)—c-Hex | —CF₂CF₃ | Z-5315 | —C(═O)—Ph | —CF₂CF₃ | Z-5316 | —C(═O)—4-OCF₃—Ph | —CF₂CF₃ |
| Z-5317 | —C(═O)—c-Hex | —CFHCF₃ | Z-5318 | —C(═O)—Ph | —CFHCF₃ | Z-5319 | —C(═O)—4-OCF₃—Ph | —CFHCF₃ |
| Z-5320 | —C(═O)—c-Hex | —CH₂CF₂CF₂H | Z-5321 | —C(═O)—Ph | —CH₂CF₂CF₂H | Z-5322 | —C(═O)—4-OCF₃—Ph | —CH₂CF₂CF₂H |
| Z-5323 | —C(═O)—c-Hex | —CH₂CF₂CF₃ | Z-5324 | —C(═O)—Ph | —CH₂CF₂CF₃ | Z-5325 | —C(═O)—4-OCF₃—Ph | —CH₂CF₂CF₃ |
| Z-5326 | —C(═O)—c-Hex | —CF₂CF₂CF₃ | Z-5327 | —C(═O)—Ph | —CF₂CF₂CF₃ | Z-5328 | —C(═O)—4-OCF₃—Ph | —CF₂CF₂CF₃ |
| Z-5329 | —C(═O)—c-Hex | —CH₂CF₂CF₂CF₃ | Z-5330 | —C(═O)—Ph | —CH₂CF₂CF₂CF₃ | Z-5331 | —C(═O)—4-OCF₃—Ph | —CH₂CF₂CF₂CF₃ |
| Z-5332 | —C(═O)—c-Hex | —CF₂CF₂CF₂CF₃ | Z-5333 | —C(═O)—Ph | —CF₂CF₂CF₂CF₃ | Z-5334 | —C(═O)—4-OCF₃—Ph | —CF₂CF₂CF₂CF₃ |
| Z-5335 | —C(═O)—c-Hex | —CH₂CF₂CF₂CF₂CF₃ | Z-5336 | —C(═O)—Ph | —CH₂CF₂CF₂CF₂CF₃ | Z-5337 | —C(═O)—4-OCF₃—Ph | —CH₂CF₂CF₂CF₂CF₃ |
| Z-5338 | —C(═O)—c-Hex | c-Pr | Z-5339 | —C(═O)—Ph | c-Pr | Z-5340 | —C(═O)—4-OCF₃—Ph | c-Pr |
| Z-5341 | —C(═O)—c-Hex | c-Bu | Z-5342 | —C(═O)—Ph | c-Bu | Z-5343 | —C(═O)—4-OCF₃—Ph | c-Bu |
| Z-5344 | —C(═O)—c-Hex | c-Pent | Z-5345 | —C(═O)—Ph | c-Pent | Z-5346 | —C(═O)—4-OCF₃—Ph | c-Pent |
| Z-5347 | —C(═O)—c-Hex | c-Hex | Z-5348 | —C(═O)—Ph | c-Hex | Z-5349 | —C(═O)—4-OCF₃—Ph | c-Hex |
| Z-5350 | —C(═O)—c-Hex | c-Hept | Z-5351 | —C(═O)—Ph | c-Hept | Z-5352 | —C(═O)—4-OCF₃—Ph | c-Hept |
| Z-5353 | —C(═O)—c-Hex | c-Oct | Z-5354 | —C(═O)—Ph | c-Oct | Z-5355 | —C(═O)—4-OCF₃—Ph | c-Oct |
| Z-5356 | —C(═O)—c-Hex | —CH═CH₂ | Z-5357 | —C(═O)—Ph | —CH═CH₂ | Z-5358 | —C(═O)—4-OCF₃—Ph | —CH═CH₂ |
| Z-5359 | —C(═O)—c-Hex | —CH₂CH═CH₂ | Z-5360 | —C(═O)—Ph | —CH₂CH═CH₂ | Z-5361 | —C(═O)—4-OCF₃—Ph | —CH₂CH═CH₂ |
| Z-5362 | —C(═O)—c-Hex | —CH═CHCH₃ | Z-5363 | —C(═O)—Ph | —CH═CHCH₃ | Z-5364 | —C(═O)—4-OCF₃—Ph | —CH═CHCH₃ |
| Z-5365 | —C(═O)—c-Hex | —CH₂C(CH₃)═CH₂ | Z-5366 | —C(═O)—Ph | —CH₂C(CH₃)═CH₂ | Z-5367 | —C(═O)—4-OCF₃—Ph | —CH₂C(CH₃)═CH₂ |
| Z-5368 | —C(═O)—c-Hex | —CH₂CH₂CH═CH₂ | Z-5369 | —C(═O)—Ph | —CH₂CH₂CH═CH₂ | Z-5370 | —C(═O)—4-OCF₃—Ph | —CH₂CH₂CH═CH₂ |
| Z-5371 | —C(═O)—c-Hex | —CH₂CH═CHCH₃ | Z-5372 | —C(═O)—Ph | —CH₂CH═CHCH₃ | Z-5373 | —C(═O)—4-OCF₃—Ph | —CH₂CH═CHCH₃ |
| Z-5374 | —C(═O)—c-Hex | —CH═CHCH₂CH₃ | Z-5375 | —C(═O)—Ph | —CH═CHCH₂CH₃ | Z-5376 | —C(═O)—4-OCF₃—Ph | —CH═CHCH₂CH₃ |
| Z-5377 | —C(═O)—c-Hex | —CH₂CH═C(CH₃)₂ | Z-5378 | —C(═O)—Ph | —CH₂CH═C(CH₃)₂ | Z-5379 | —C(═O)—4-OCF₃—Ph | —CH₂CH═C(CH₃)₂ |
| Z-5380 | —C(═O)—c-Hex | —CH₂CH₂CH═C(CH₃)₂ | Z-5381 | —C(═O)—Ph | —CH₂CH₂CH═C(CH₃)₂ | Z-5382 | —C(═O)—4-OCF₃—Ph | —CH₂CH₂CH═C(CH₃)₂ |
| Z-5383 | —C(═O)—c-Hex | —CH═CFH | Z-5384 | —C(═O)—Ph | —CH═CFH | Z-5385 | —C(═O)—4-OCF₃—Ph | —CH═CFH |
| Z-5386 | —C(═O)—c-Hex | —CH═CF₂ | Z-5387 | —C(═O)—Ph | —CH═CF₂ | Z-5388 | —C(═O)—4-OCF₃—Ph | —CH═CF₂ |
| Z-5389 | —C(═O)—c-Hex | —CH═CCl₂ | Z-5390 | —C(═O)—Ph | —CH═CCl₂ | Z-5391 | —C(═O)—4-OCF₃—Ph | —CH═CCl₂ |
| Z-5392 | —C(═O)—c-Hex | —CH₂CH═CFH | Z-5393 | —C(═O)—Ph | —CH₂CH═CFH | Z-5394 | —C(═O)—4-OCF₃—Ph | —CH₂CH═CFH |
| Z-5395 | —C(═O)—c-Hex | —CH₂CH═CF₂ | Z-5396 | —C(═O)—Ph | —CH₂CH═CF₂ | Z-5397 | —C(═O)—4-OCF₃—Ph | —CH₂CH═CF₂ |
| Z-5398 | —C(═O)—c-Hex | —CH₂CH═CCl₂ | Z-5399 | —C(═O)—Ph | —CH₂CH═CCl₂ | Z-5400 | —C(═O)—4-OCF₃—Ph | —CH₂CH═CCl₂ |
| Z-5401 | —C(═O)—c-Hex | —CH₂CH₂CH═CF₂ | Z-5402 | —C(═O)—Ph | —CH₂CH₂CH═CF₂ | Z-5403 | —C(═O)—4-OCF₃—Ph | —CH₂CH₂CH═CF₂ |
| Z-5404 | —C(═O)—c-Hex | —CH₂CH₂CH₂CH═CF₂ | Z-5405 | —C(═O)—Ph | —CH₂CH₂CH₂CH═CF₂ | Z-5406 | —C(═O)—4-OCF₃—Ph | —CH₂CH₂CH₂CH═CF₂ |
| Z-5407 | —C(═O)—c-Hex | —CH₂CH₂CH₂CH₂CH═CF₂ | Z-5408 | —C(═O)—Ph | —CH₂CH₂CH₂CH₂CH═CF₂ | Z-5409 | —C(═O)—4-OCF₃—Ph | —CH₂CH₂CH₂CH₂CH═CF₂ |
| Z-5410 | —C(═O)—c-Hex | —C≡CH | Z-5411 | —C(═O)—Ph | —C≡CH | Z-5412 | —C(═O)—4-OCF₃—Ph | —C≡CH |
| Z-5413 | —C(═O)—c-Hex | —C≡CCH₃ | Z-5414 | —C(═O)—Ph | —C≡CCH₃ | Z-5415 | —C(═O)—4-OCF₃—Ph | —C≡CCH₃ |
| Z-5416 | —C(═O)—c-Hex | —CH₂C≡CH | Z-5417 | —C(═O)—Ph | —CH₂C≡CH | Z-5418 | —C(═O)—4-OCF₃—Ph | —CH₂C≡CH |
| Z-5419 | —C(═O)—c-Hex | —C≡CCH₂CH₃ | Z-5420 | —C(═O)—Ph | —C≡CCH₂CH₃ | Z-5421 | —C(═O)—4-OCF₃—Ph | —C≡CCH₂CH₃ |
| Z-5422 | —C(═O)—c-Hex | —CH₂C≡CCH₃ | Z-5423 | —C(═O)—Ph | —CH₂C≡CCH₃ | Z-5424 | —C(═O)—4-OCF₃—Ph | —CH₂C≡CCH₃ |
| Z-5425 | —C(═O)—c-Hex | —CH₂CH₂C≡CH | Z-5426 | —C(═O)—Ph | —CH₂CH₂C≡CH | Z-5427 | —C(═O)—4-OCF₃—Ph | —CH₂CH₂C≡CH |
| Z-5428 | —C(═O)—c-Hex | —C≡CCH₂CH₂CH₃ | Z-5429 | —C(═O)—Ph | —C≡CCH₂CH₂CH₃ | Z-5430 | —C(═O)—4-OCF₃—Ph | —C≡CCH₂CH₂CH₃ |
| Z-5431 | —C(═O)—c-Hex | —CH₂C≡CCH₂CH₃ | Z-5432 | —C(═O)—Ph | —CH₂C≡CCH₂CH₃ | Z-5433 | —C(═O)—4-OCF₃—Ph | —CH₂C≡CCH₂CH₃ |
| Z-5434 | —C(═O)—c-Hex | —C(CH₃)₂C≡CH | Z-5435 | —C(═O)—Ph | —C(CH₃)₂C≡CH | Z-5436 | —C(═O)—4-OCF₃—Ph | —C(CH₃)₂C≡CH |
| Z-5437 | —C(═O)—c-Hex | —C≡CF | Z-5438 | —C(═O)—Ph | —C≡CF | Z-5439 | —C(═O)—4-OCF₃—Ph | —C≡CF |
| Z-5440 | —C(═O)—c-Hex | —C≡CCF₂H | Z-5441 | —C(═O)—Ph | —C≡CCF₂H | Z-5442 | —C(═O)—4-OCF₃—Ph | —C≡CCF₂H |
| Z-5443 | —C(═O)—c-Hex | —C≡CCF₃ | Z-5444 | —C(═O)—Ph | —C≡CCF₃ | Z-5445 | —C(═O)—4-OCF₃—Ph | —C≡CCF₃ |
| Z-5446 | —C(═O)—c-Hex | —C≡CCH₂CF₂H | Z-5447 | —C(═O)—Ph | —C≡CCH₂CF₂H | Z-5448 | —C(═O)—4-OCF₃—Ph | —C≡CCH₂CF₂H |
| Z-5449 | —C(═O)—c-Hex | —C≡CCH₂CF₃ | Z-5450 | —C(═O)—Ph | —C≡CCH₂CF₃ | Z-5451 | —C(═O)—4-OCF₃—Ph | —C≡CCH₂CF₃ |
| Z-5452 | —C(═O)—c-Hex | —CH₂C≡CHCF₂H | Z-5453 | —C(═O)—Ph | —CH₂C≡CHCF₂H | Z-5454 | —C(═O)—4-OCF₃—Ph | —CH₂C≡CHCF₂H |
| Z-5455 | —C(═O)—c-Hex | —CH₂C≡CCF₃ | Z-5456 | —C(═O)—Ph | —CH₂C≡CCF₃ | Z-5457 | —C(═O)—4-OCF₃—Ph | —CH₂C≡CCF₃ |
| Z-5458 | —C(═O)—c-Hex | —C(═O)NH₂ | Z-5459 | —C(═O)—Ph | —C(═O)NH₂ | Z-5460 | —C(═O)—4-OCF₃—Ph | —C(═O)NH₂ |
| Z-5461 | —C(═O)—c-Hex | —C(═O)NHMe | Z-5462 | —C(═O)—Ph | —C(═O)NHMe | Z-5463 | —C(═O)—4-OCF₃—Ph | —C(═O)NHMe |
| Z-5464 | —C(═O)—c-Hex | —C(═O)NHEt | Z-5465 | —C(═O)—Ph | —C(═O)NHEt | Z-5466 | —C(═O)—4-OCF₃—Ph | —C(═O)NHEt |
| Z-5467 | —C(═O)—c-Hex | —C(═O)NHPr | Z-5468 | —C(═O)—Ph | —C(═O)NHPr | Z-5469 | —C(═O)—4-OCF₃—Ph | —C(═O)NHPr |
| Z-5470 | —C(═O)—c-Hex | —C(═O)NH—i-Pr | Z-5471 | —C(═O)—Ph | —C(═O)NH—i-Pr | Z-5472 | —C(═O)—4-OCF₃—Ph | —C(═O)NH—i-Pr |
| Z-5473 | —C(═O)—c-Hex | —C(═O)NHBu | Z-5474 | —C(═O)—Ph | —C(═O)NHBu | Z-5475 | —C(═O)—4-OCF₃—Ph | —C(═O)NHBu |
| Z-5476 | —C(═O)—c-Hex | —C(═O)NH—sec-Bu | Z-5477 | —C(═O)—Ph | —C(═O)NH—sec-Bu | Z-5478 | —C(═O)—4-OCF₃—Ph | —C(═O)NH—sec-Bu |
| Z-5479 | —C(═O)—c-Hex | —C(═O)NH—i-Bu | Z-5480 | —C(═O)—Ph | —C(═O)NH—i-Bu | Z-5481 | —C(═O)—4-OCF₃—Ph | —C(═O)NH—i-Bu |
| Z-5482 | —C(═O)—c-Hex | —C(═O)NH—t-Bu | Z-5483 | —C(═O)—Ph | —C(═O)NH—t-Bu | Z-5484 | —C(═O)—4-OCF₃—Ph | —C(═O)NH—t-Bu |
| Z-5485 | —C(═O)—c-Hex | —C(═O)NHPent | Z-5486 | —C(═O)—Ph | —C(═O)NHPent | Z-5487 | —C(═O)—4-OCF₃—Ph | —C(═O)NHPent |
| Z-5488 | —C(═O)—c-Hex | —C(═O)NHHex | Z-5489 | —C(═O)—Ph | —C(═O)NHHex | Z-5490 | —C(═O)—4-OCF₃—Ph | —C(═O)NHHex |
| Z-5491 | —C(═O)—c-Hex | —C(═O)NHCH₂C≡N | Z-5492 | —C(═O)—Ph | —C(═O)NHCH₂C≡N | Z-5493 | —C(═O)—4-OCF₃—Ph | —C(═O)NHCH₂C≡N |
| Z-5494 | —C(═O)—c-Hex | —C(═O)NHCH₂—c-Pr | Z-5495 | —C(═O)—Ph | —C(═O)NHCH₂—c-Pr | Z-5496 | —C(═O)—4-OCF₃—Ph | —C(═O)NHCH₂—c-Pr |
| Z-5497 | —C(═O)—c-Hex | —C(═O)NHCH₂OMe | Z-5498 | —C(═O)—Ph | —C(═O)NHCH₂OMe | Z-5499 | —C(═O)—4-OCF₃—Ph | —C(═O)NHCH₂OMe |
| Z-5500 | —C(═O)—c-Hex | —C(═O)NHCH₂CH₂OMe | Z-5501 | —C(═O)—Ph | —C(═O)NHCH₂CH₂OMe | Z-5502 | —C(═O)—4-OCF₃—Ph | —C(═O)NHCH₂CH₂OMe |
| Z-5503 | —C(═O)—c-Hex | —C(═O)NHCFH₂ | Z-5504 | —C(═O)—Ph | —C(═O)NHCFH₂ | Z-5505 | —C(═O)—4-OCF₃—Ph | —C(═O)NHCFH₂ |
| Z-5506 | —C(═O)—c-Hex | —C(═O)NHCF₂H | Z-5507 | —C(═O)—Ph | —C(═O)NHCF₂H | Z-5508 | —C(═O)—4-OCF₃—Ph | —C(═O)NHCF₂H |
| Z-5509 | —C(═O)—c-Hex | —C(═O)NHCF₃ | Z-5510 | —C(═O)—Ph | —C(═O)NHCF₃ | Z-5511 | —C(═O)—4-OCF₃—Ph | —C(═O)NHCF₃ |
| Z-5512 | —C(═O)—c-Hex | —C(═O)NHCH₂Cl | Z-5513 | —C(═O)—Ph | —C(═O)NHCH₂Cl | Z-5514 | —C(═O)—4-OCF₃—Ph | —C(═O)NHCH₂Cl |
| Z-5515 | —C(═O)—c-Hex | —C(═O)NHCHCl₂ | Z-5516 | —C(═O)—Ph | —C(═O)NHCHCl₂ | Z-5517 | —C(═O)—4-OCF₃—Ph | —C(═O)NHCHCl₂ |
| Z-5518 | —C(═O)—c-Hex | —C(═O)NHCCl₃ | Z-5519 | —C(═O)—Ph | —C(═O)NHCCl₃ | Z-5520 | —C(═O)—4-OCF₃—Ph | —C(═O)NHCCl₃ |
| Z-5521 | —C(═O)—c-Hex | —C(═O)NHCH₂Br | Z-5522 | —C(═O)—Ph | —C(═O)NHCH₂Br | Z-5523 | —C(═O)—4-OCF₃—Ph | —C(═O)NHCH₂Br |
| Z-5524 | —C(═O)—c-Hex | —C(═O)NHCHBr₂ | Z-5525 | —C(═O)—Ph | —C(═O)NHCHBr₂ | Z-5526 | —C(═O)—4-OCF₃—Ph | —C(═O)NHCHBr₂ |
| Z-5527 | —C(═O)—c-Hex | —C(═O)NHCBr₃ | Z-5528 | —C(═O)—Ph | —C(═O)NHCBr₃ | Z-5529 | —C(═O)—4-OCF₃—Ph | —C(═O)NHCBr₃ |
| Z-5530 | —C(═O)—c-Hex | —C(═O)NHCH₂l | Z-5531 | —C(═O)—Ph | —C(═O)NHCH₂l | Z-5532 | —C(═O)—4-OCF₃—Ph | —C(═O)NHCH₂l |
| Z-5533 | —C(═O)—c-Hex | —C(═O)NHCHl₂ | Z-5534 | —C(═O)—Ph | —C(═O)NHCHl₂ | Z-5535 | —C(═O)—4-OCF₃—Ph | —C(═O)NHCHl₂ |
| Z-5536 | —C(═O)—c-Hex | —C(═O)NHCH₂CF₂H | Z-5537 | —C(═O)—Ph | —C(═O)NHCH₂CF₂H | Z-5538 | —C(═O)—4-OCF₃—Ph | —C(═O)NHCH₂CF₂H |
| Z-5539 | —C(═O)—c-Hex | —C(═O)NHCH₂CF₃ | Z-5540 | —C(═O)—Ph | —C(═O)NHCH₂CF₃ | Z-5541 | —C(═O)—4-OCF₃—Ph | —C(═O)NHCH₂CF₃ |
| Z-5542 | —C(═O)—c-Hex | —C(═O)NHCH₂CH₂CF₂H | Z-5543 | —C(═O)—Ph | —C(═O)NHCH₂CH₂CF₂H | Z-5544 | —C(═O)—4-OCF₃—Ph | —C(═O)NHCH₂CH₂CF₂H |
| Z-5545 | —C(═O)—c-Hex | —C(═O)NHCH₂CH₂CF₃ | Z-5546 | —C(═O)—Ph | —C(═O)NHCH₂CH₂CF₃ | Z-5547 | —C(═O)—4-OCF₃—Ph | —C(═O)NHCH₂CH₂CF₃ |
| Z-5548 | —C(═O)—c-Hex | —C(═O)NHCH₂CH₂CH₂CF₂H | Z-5549 | —C(═O)—Ph | —C(═O)NHCH₂CH₂CH₂CF₂H | Z-5550 | —C(═O)—4-OCF₃—Ph | —C(═O)NHCH₂CH₂CH₂CF₂H |
| Z-5551 | —C(═O)—c-Hex | —C(═O)NHCH₂CH₂CH₂CF₃ | Z-5552 | —C(═O)—Ph | —C(═O)NHCH₂CH₂CH₂CF₃ | Z-5553 | —C(═O)—4-OCF₃—Ph | —C(═O)NHCH₂CH₂CH₂CF₃ |
| Z-5554 | —C(═O)—c-Hex | —C(═O)NHCF₂CF₂H | Z-5555 | —C(═O)—Ph | —C(═O)NHCF₂CF₂H | Z-5556 | —C(═O)—4-OCF₃—Ph | —C(═O)NHCF₂CF₂H |
| Z-5557 | —C(═O)—c-Hex | —C(═O)NHCF₂CF₃ | Z-5558 | —C(═O)—Ph | —C(═O)NHCF₂CF₃ | Z-5559 | —C(═O)—4-OCF₃—Ph | —C(═O)NHCF₂CF₃ |
| Z-5560 | —C(═O)—c-Hex | —C(═O)NHCFHCF₃ | Z-5561 | —C(═O)—Ph | —C(═O)NHCFHCF₃ | Z-5562 | —C(═O)—4-OCF₃—Ph | —C(═O)NHCFHCF₃ |
| Z-5563 | —C(═O)—c-Hex | —C(═O)NHCH₂CF₂CF₂H | Z-5564 | —C(═O)—Ph | —C(═O)NHCH₂CF₂CF₂H | Z-5565 | —C(═O)—4-OCF₃—Ph | —C(═O)NHCH₂CF₂CF₂H |
| Z-5566 | —C(═O)—c-Hex | —C(═O)NHCH₂CF₂CF₃ | Z-5567 | —C(═O)—Ph | —C(═O)NHCH₂CF₂CF₃ | Z-5568 | —C(═O)—4-OCF₃—Ph | —C(═O)NHCH₂CF₂CF₃ |
| Z-5569 | —C(═O)—c-Hex | —C(═O)NHCF₂CF₂CF₃ | Z-5570 | —C(═O)—Ph | —C(═O)NHCF₂CF₂CF₃ | Z-5571 | —C(═O)—4-OCF₃—Ph | —C(═O)NHCF₂CF₂CF₃ |
| Z-5572 | —C(═O)—c-Hex | —C(═O)NHCH₂CF₂CF₂CF₃ | Z-5573 | —C(═O)—Ph | —C(═O)NHCH₂CF₂CF₂CF₃ | Z-5574 | —C(═O)—4-OCF₃—Ph | —C(═O)NHCH₂CF₂CF₂CF₃ |
| Z-5575 | —C(═O)—c-Hex | —C(═O)NHCF₂CF₂CF₂CF₃ | Z-5576 | —C(═O)—Ph | —C(═O)NHCF₂CF₂CF₂CF₃ | Z-5577 | —C(═O)—4-OCF₃—Ph | —C(═O)NHCF₂CF₂CF₂CF₃ |
| Z-5578 | —C(═O)—c-Hex | —C(═O)NHCH₂CF₂CF₂CF₂CF₃ | Z-5579 | —C(═O)—Ph | —C(═O)NHCH₂CF₂CF₂CF₂CF₃ | Z-5580 | —C(═O)—4-OCF₃—Ph | —C(═O)NHCH₂CF₂CF₂CF₂CF₃ |
| Z-5581 | —C(═O)—c-Hex | —C(═O)NH—c-Pr | Z-5582 | —C(═O)—Ph | —C(═O)NH—c-Pr | Z-5583 | —C(═O)—4-OCF₃—Ph | —C(═O)NH—c-Pr |
| Z-5584 | —C(═O)—c-Hex | —C(═O)NH—c-Bu | Z-5585 | —C(═O)—Ph | —C(═O)NH—c-Bu | Z-5586 | —C(═O)—4-OCF₃—Ph | —C(═O)NH—c-Bu |
| Z-5587 | —C(═O)—c-Hex | —C(═O)NH—c-Pent | Z-5588 | —C(═O)—Ph | —C(═O)NH—c-Pent | Z-5589 | —C(═O)—4-OCF₃—Ph | —C(═O)NH—c-Pent |
| Z-5590 | —C(═O)—c-Hex | —C(═O)NH—c-Hex | Z-5591 | —C(═O)—Ph | —C(═O)NH—c-Hex | Z-5592 | —C(═O)—4-OCF₃—Ph | —C(═O)NH—c-Hex |
| Z-5593 | —C(═O)—c-Hex | —C(═O)NH—c-Hept | Z-5594 | —C(═O)—Ph | —C(═O)NH—c-Hept | Z-5595 | —C(═O)—4-OCF₃—Ph | —C(═O)NH—c-Hept |
| Z-5596 | —C(═O)—c-Hex | —C(═O)NH—c-Oct | Z-5597 | —C(═O)—Ph | —C(═O)NH—c-Oct | Z-5598 | —C(═O)—4-OCF₃—Ph | —C(═O)NH—c-Oct |
| Z-5599 | —C(═O)—c-Hex | —C(═O)NHCH₂CH═CH₂ | Z-5600 | —C(═O)—Ph | —C(═O)NHCH₂CH═CH₂ | Z-5601 | —C(═O)—4-OCF₃—Ph | —C(═O)NHCH₂CH═CH₂ |
| Z-5602 | —C(═O)—c-Hex | —C(═O)NHCH₂C(CH₃)═CH₂ | Z-5603 | —C(═O)—Ph | —C(═O)NHCH₂C(CH₃)═CH₂ | Z-5604 | —C(═O)—4-OCF₃—Ph | —C(═O)NHCH₂C(CH₃)═CH₂ |
| Z-5605 | —C(═O)—c-Hex | —C(═O)NHCH₂CH₂CH═CH₂ | Z-5606 | —C(═O)—Ph | —C(═O)NHCH₂CH₂CH═CH₂ | Z-5607 | —C(═O)—4-OCF₃—Ph | —C(═O)NHCH₂CH₂CH═CH₂ |
| Z-5608 | —C(═O)—c-Hex | —C(═O)NHCH₂CH═CHCH₃ | Z-5609 | —C(═O)—Ph | —C(═O)NHCH₂CH═CHCH₃ | Z-5610 | —C(═O)—4-OCF₃—Ph | —C(═O)NHCH₂CH═CHCH₃ |
| Z-5611 | —C(═O)—c-Hex | —C(═O)NHCH₂CH═C(CH₃)₂ | Z-5612 | —C(═O)—Ph | —C(═O)NHCH₂CH═C(CH₃)₂ | Z-5613 | —C(═O)—4-OCF₃—Ph | —C(═O)NHCH₂CH═C(CH₃)₂ |
| Z-5614 | —C(═O)—c-Hex | —C(═O)NHCH₂CH₂CH═C(CH₃)₂ | Z-5615 | —C(═O)—Ph | —C(═O)NHCH₂CH₂CH═C(CH₃)₂ | Z-5616 | —C(═O)—4-OCF₃—Ph | —C(═O)NHCH₂CH₂CH═C(CH₃)₂ |
| Z-5617 | —C(═O)—c-Hex | —C(═O)NHCH₂CH═CFH | Z-5618 | —C(═O)—Ph | —C(═O)NHCH₂CH═CFH | Z-5619 | —C(═O)—4-OCF₃—Ph | —C(═O)NHCH₂CH═CFH |
| Z-5620 | —C(═O)—c-Hex | —C(═O)NHCH₂CH═CF₂ | Z-5621 | —C(═O)—Ph | —C(═O)NHCH₂CH═CF₂ | Z-5622 | —C(═O)—4-OCF₃—Ph | —C(═O)NHCH₂CH═CF₂ |
| Z-5623 | —C(═O)—c-Hex | —C(═O)NHCH₂CH═CCl₂ | Z-5624 | —C(═O)—Ph | —C(═O)NHCH₂CH═CCl₂ | Z-5625 | —C(═O)—4-OCF₃—Ph | —C(═O)NHCH₂CH═CCl₂ |
| Z-5626 | —C(═O)—c-Hex | —C(═O)NHCH₂CH₂CH═CF₂ | Z-5627 | —C(═O)—Ph | —C(═O)NHCH₂CH₂CH═CF₂ | Z-5628 | —C(═O)—4-OCF₃—Ph | —C(═O)NHCH₂CH₂CH═CF₂ |
| Z-5629 | —C(═O)—c-Hex | —C(═O)NHCH₂CH₂CH₂CH═CF₂ | Z-5630 | —C(═O)—Ph | —C(═O)NHCH₂CH₂CH₂CH═CF₂ | Z-5631 | —C(═O)—4-OCF₃—Ph | —C(═O)NHCH₂CH₂CH₂CH═CF₂ |
| Z-5632 | —C(═O)—c-Hex | —C(═O)NHCH₂CH₂CH₂CH₂CH═CF₂ | Z-5633 | —C(═O)—Ph | —C(═O)NHCH₂CH₂CH₂CH₂CH═CF₂ | Z-5634 | —C(═O)—4-OCF₃—Ph | —C(═O)NHCH₂CH₂CH₂CH₂CH═CF₂ |
| Z-5635 | —C(═O)—c-Hex | —C(═O)NHCH₂C≡CH | Z-5636 | —C(═O)—Ph | —C(═O)NHCH₂C≡CH | Z-5637 | —C(═O)—4-OCF₃—Ph | —C(═O)NHCH₂C≡CH |
| Z-5638 | —C(═O)—c-Hex | —C(═O)NHCH₂C≡CCH₃ | Z-5639 | —C(═O)—Ph | —C(═O)NHCH₂C≡CCH₃ | Z-5640 | —C(═O)—4-OCF₃—Ph | —C(═O)NHCH₂C≡CCH₃ |
| Z-5641 | —C(═O)—c-Hex | —C(═O)NHCH₂CH₂C≡CH | Z-5642 | —C(═O)—Ph | —C(═O)NHCH₂CH₂C≡CH | Z-5643 | —C(═O)—4-OCF₃—Ph | —C(═O)NHCH₂CH₂C≡CH |
| Z-5644 | —C(═O)—c-Hex | —C(═O)NHCH₂C≡CCH₂CH₃ | Z-5645 | —C(═O)—Ph | —C(═O)NHCH₂C≡CCH₂CH₃ | Z-5646 | —C(═O)—4-OCF₃—Ph | —C(═O)NHCH₂C≡CCH₂CH₃ |
| Z-5647 | —C(═O)—c-Hex | —C(═O)NHC(CH₃)₂C≡CH | Z-5648 | —C(═O)—Ph | —C(═O)NHC(CH₃)₂C≡CH | Z-5649 | —C(═O)—4-OCF₃—Ph | —C(═O)NHC(CH₃)₂C≡CH |
| Z-5650 | —C(═O)—c-Hex | —C(═O)NHCH₂C≡CHCF₂H | Z-5651 | —C(═O)—Ph | —C(═O)NHCH₂C≡CHCF₂H | Z-5652 | —C(═O)—4-OCF₃—Ph | —C(═O)NHCH₂C≡CHCF₂H |
| Z-5653 | —C(═O)—c-Hex | —C(═O)NHCH₂C≡CCF₃ | Z-5654 | —C(═O)—Ph | —C(═O)NHCH₂C≡CCF₃ | Z-5655 | —C(═O)—4-OCF₃—Ph | —C(═O)NHCH₂C≡CCF₃ |
| Z-5656 | —C(═O)—c-Hex | —C(═O)H | Z-5657 | —C(═O)—Ph | —C(═O)H | Z-5658 | —C(═O)—4-OCF₃—Ph | —C(═O)H |
| Z-5659 | —C(═O)—c-Hex | —C(═O)Me | Z-5660 | —C(═O)—Ph | —C(═O)Me | Z-5661 | —C(═O)—4-OCF₃—Ph | —C(═O)Me |
| Z-5662 | —C(═O)—c-Hex | —C(═O)Et | Z-5663 | —C(═O)—Ph | —C(═O)Et | Z-5664 | —C(═O)—4-OCF₃—Ph | —C(═O)Et |
| Z-5665 | —C(═O)—c-Hex | —C(═O)Pr | Z-5666 | —C(═O)—Ph | —C(═O)Pr | Z-5667 | —C(═O)—4-OCF₃—Ph | —C(═O)Pr |
| Z-5668 | —C(═O)—c-Hex | —C(═O)—i-Pr | Z-5669 | —C(═O)—Ph | —C(═O)—i-Pr | Z-5670 | —C(═O)—4-OCF₃—Ph | —C(═O)—i-Pr |
| Z-5671 | —C(═O)—c-Hex | —C(═O)Bu | Z-5672 | —C(═O)—Ph | —C(═O)Bu | Z-5673 | —C(═O)—4-OCF₃—Ph | —C(═O)Bu |
| Z-5674 | —C(═O)—c-Hex | —C(═O)—sec-Bu | Z-5675 | —C(═O)—Ph | —C(═O)—sec-Bu | Z-5676 | —C(═O)—4-OCF₃—Ph | —C(═O)—sec-Bu |
| Z-5677 | —C(═O)—c-Hex | —C(═O)—i-Bu | Z-5678 | —C(═O)—Ph | —C(═O)—i-Bu | Z-5679 | —C(═O)—4-OCF₃—Ph | —C(═O)—i-Bu |
| Z-5680 | —C(═O)—c-Hex | —C(═O)—t-Bu | Z-5681 | —C(═O)—Ph | —C(═O)—t-Bu | Z-5682 | —C(═O)—4-OCF₃—Ph | —C(═O)—t-Bu |
| Z-5683 | —C(═O)—c-Hex | —C(═O)Pent | Z-5684 | —C(═O)—Ph | —C(═O)Pent | Z-5685 | —C(═O)—4-OCF₃—Ph | —C(═O)Pent |
| Z-5686 | —C(═O)—c-Hex | —C(═O)Hex | Z-5687 | —C(═O)—Ph | —C(═O)Hex | Z-5688 | —C(═O)—4-OCF₃—Ph | —C(═O)Hex |
| Z-5689 | —C(═O)—c-Hex | —C(═O)CH(CH₃)CH₂CH₂CH₃ | Z-5690 | —C(═O)—Ph | —C(═O)CH(CH₃)CH₂CH₂CH₃ | Z-5691 | —C(═O)—4-OCF₃—Ph | —C(═O)CH(CH₃)CH₂CH₂CH₃ |
| Z-5692 | —C(═O)—c-Hex | —C(═O)CH(CH₃)CH(CH₃)₂ | Z-5693 | —C(═O)—Ph | —C(═O)CH(CH₃)CH(CH₃)₂ | Z-5694 | —C(═O)—4-OCF₃—Ph | —C(═O)CH(CH₃)CH(CH₃)₂ |
| Z-5695 | —C(═O)—c-Hex | —C(═O)C(CH₃)₂CH₂CH₃ | Z-5696 | —C(═O)—Ph | —C(═O)C(CH₃)₂CH₂CH₃ | Z-5697 | —C(═O)—4-OCF₃—Ph | —C(═O)C(CH₃)₂CH₂CH₃ |
| Z-5698 | —C(═O)—c-Hex | —C(═O)CH(CH₂CH₃)₂ | Z-5699 | —C(═O)—Ph | —C(═O)CH(CH₂CH₃)₂ | Z-5700 | —C(═O)—4-OCF₃—Ph | —C(═O)CH(CH₂CH₃)₂ |
| Z-5701 | —C(═O)—c-Hex | —C(═O)CH₂CH₂CH(CH₃)₂ | Z-5702 | —C(═O)—Ph | —C(═O)CH₂CH₂CH(CH₃)₂ | Z-5703 | —C(═O)—4-OCF₃—Ph | —C(═O)CH₂CH₂CH(CH₃)₂ |
| Z-5704 | —C(═O)—c-Hex | —C(═O)CH₂CH₂CH₂CH(CH₃)₂ | Z-5705 | —C(═O)—Ph | —C(═O)CH₂CH₂CH₂CH(CH₃)₂ | Z-5706 | —C(═O)—4-OCF₃—Ph | —C(═O)CH₂CH₂CH₂CH(CH₃)₂ |
| Z-5707 | —C(═O)—c-Hex | —C(═O)CH₂CH₂CH(CH₃)CH₂CH₃ | Z-5708 | —C(═O)—Ph | —C(═O)CH₂CH₂CH(CH₃)CH₂CH₃ | Z-5709 | —C(═O)—4-OCF₃—Ph | —C(═O)CH₂CH₂CH(CH₃)CH₂CH₃ |
| Z-5710 | —C(═O)—c-Hex | —C(═O)CH₂CH(CH₃)CH₂CH₂CH₃ | Z-5711 | —C(═O)—Ph | —C(═O)CH₂CH(CH₃)CH₂CH₂CH₃ | Z-5712 | —C(═O)—4-OCF₃—Ph | —C(═O)CH₂CH(CH₃)CH₂CH₂CH₃ |
| Z-5713 | —C(═O)—c-Hex | —C(═O)CH(CH₃)CH₂CH₂CH₂CH₃ | Z-5714 | —C(═O)—Ph | —C(═O)CH(CH₃)CH₂CH₂CH₂CH₃ | Z-5715 | —C(═O)—4-OCF₃—Ph | —C(═O)CH(CH₃)CH₂CH₂CH₂CH₃ |
| Z-5716 | —C(═O)—c-Hex | —C(═O)CH(CH₃)CH₂CH(CH₃)₂ | Z-5717 | —C(═O)—Ph | —C(═O)CH(CH₃)CH₂CH(CH₃)₂ | Z-5718 | —C(═O)—4-OCF₃—Ph | —C(═O)CH(CH₃)CH₂CH(CH₃)₂ |
| Z-5719 | —C(═O)—c-Hex | —C(═O)CH(CH₃)CH(CH₃)CH₂CH₃ | Z-5720 | —C(═O)—Ph | —C(═O)CH(CH₃)CH(CH₃)CH₂CH₃ | Z-5721 | —C(═O)—4-OCF₃—Ph | —C(═O)CH(CH₃)CH(CH₃)CH₂CH₃ |
| Z-5722 | —C(═O)—c-Hex | —C(═O)C(CH₃)₂CH₂CH₂CH₃ | Z-5723 | —C(═O)—Ph | —C(═O)C(CH₃)₂CH₂CH₂CH₃ | Z-5724 | —C(═O)—4-OCF₃—Ph | —C(═O)C(CH₃)₂CH₂CH₂CH₃ |
| Z-5725 | —C(═O)—c-Hex | —C(═O)CH(CH₃)C(CH₃)₃ | Z-5726 | —C(═O)—Ph | —C(═O)CH(CH₃)C(CH₃)₃ | Z-5727 | —C(═O)—4-OCF₃—Ph | —C(═O)CH(CH₃)C(CH₃)₃ |
| Z-5728 | —C(═O)—c-Hex | —C(═O)C(CH₃)₂CH(CH₃)₂ | Z-5729 | —C(═O)—Ph | —C(═O)C(CH₃)₂CH(CH₃)₂ | Z-5730 | —C(═O)—4-OCF₃—Ph | —C(═O)C(CH₃)₂CH(CH₃)₂ |
| Z-5731 | —C(═O)—c-Hex | —C(═O)CH₂CH₂C(CH₃) | Z-5732 | —C(═O)—Ph | —C(═O)CH₂CH₂C(CH₃) | Z-5733 | —C(═O)—4-OCF₃—Ph | —C(═O)CH₂CH₂C(CH₃) |
| Z-5734 | —C(═O)—c-Hex | —C(═O)CH₂CH(CH₃)CH(CH₃)₂ | Z-5735 | —C(═O)—Ph | —C(═O)CH₂CH(CH₃)CH(CH₃)₂ | Z-5736 | —C(═O)—4-OCF₃—Ph | —C(═O)CH₂CH(CH₃)CH(CH₃)₂ |
| Z-5737 | —C(═O)—c-Hex | —C(═O)CH₂C(CH₃)₂CH₂CH₃ | Z-5738 | —C(═O)—Ph | —C(═O)CH₂C(CH₃)₂CH₂CH₃ | Z-5739 | —C(═O)—4-OCF₃—Ph | —C(═O)CH₂C(CH₃)₂CH₂CH₃ |
| Z-5740 | —C(═O)—c-Hex | —C(═O)CFH₂ | Z-5741 | —C(═O)—Ph | —C(═O)CFH₂ | Z-5742 | —C(═O)—4-OCF₃—Ph | —C(═O)CFH₂ |
| Z-5743 | —C(═O)—c-Hex | —C(═O)CF₂H | Z-5744 | —C(═O)—Ph | —C(═O)CF₂H | Z-5745 | —C(═O)—4-OCF₃—Ph | —C(═O)CF₂H |
| Z-5746 | —C(═O)—c-Hex | —C(═O)CF₂Cl | Z-5747 | —C(═O)—Ph | —C(═O)CF₂Cl | Z-5748 | —C(═O)—4-OCF₃—Ph | —C(═O)CF₂Cl |
| Z-5749 | —C(═O)—c-Hex | —C(═O)CF₃ | Z-5750 | —C(═O)—Ph | —C(═O)CF₃ | Z-5751 | —C(═O)—4-OCF₃—Ph | —C(═O)CF₃ |
| Z-5752 | —C(═O)—c-Hex | —C(═O)CH₂Cl | Z-5753 | —C(═O)—Ph | —C(═O)CH₂Cl | Z-5754 | —C(═O)—4-OCF₃—Ph | —C(═O)CH₂Cl |
| Z-5755 | —C(═O)—c-Hex | —C(═O)CHCl₂ | Z-5756 | —C(═O)—Ph | —C(═O)CHCl₂ | Z-5757 | —C(═O)—4-OCF₃—Ph | —C(═O)CHCl₂ |
| Z-5758 | —C(═O)—c-Hex | —C(═O)CCl₂F | Z-5759 | —C(═O)—Ph | —C(═O)CCl₂F | Z-5760 | —C(═O)—4-OCF₃—Ph | —C(═O)CCl₂F |
| Z-5761 | —C(═O)—c-Hex | —C(═O)CCl₃ | Z-5762 | —C(═O)—Ph | —C(═O)CCl₃ | Z-5763 | —C(═O)—4-OCF₃—Ph | —C(═O)CCl₃ |
| Z-5764 | —C(═O)—c-Hex | —C(═O)CH₂Br | Z-5765 | —C(═O)—Ph | —C(═O)CH₂Br | Z-5766 | —C(═O)—4-OCF₃—Ph | —C(═O)CH₂Br |
| Z-5767 | —C(═O)—c-Hex | —C(═O)CHBr₂ | Z-5768 | —C(═O)—Ph | —C(═O)CHBr₂ | Z-5769 | —C(═O)—4-OCF₃—Ph | —C(═O)CHBr₂ |
| Z-5770 | —C(═O)—c-Hex | —C(═O)CBr₃ | Z-5771 | —C(═O)—Ph | —C(═O)CBr₃ | Z-5772 | —C(═O)—4-OCF₃—Ph | —C(═O)CBr₃ |
| Z-5773 | —C(═O)—c-Hex | —C(═O)CH₂l | Z-5774 | —C(═O)—Ph | —C(═O)CH₂l | Z-5775 | —C(═O)—4-OCF₃—Ph | —C(═O)CH₂l |
| Z-5776 | —C(═O)—c-Hex | —C(═O)CHl₂ | Z-5777 | —C(═O)—Ph | —C(═O)CHl₂ | Z-5778 | —C(═O)—4-OCF₃—Ph | —C(═O)CHl₂ |
| Z-5779 | —C(═O)—c-Hex | —C(═O)CH₂CF₂H | Z-5780 | —C(═O)—Ph | —C(═O)CH₂CF₂H | Z-5781 | —C(═O)—4-OCF₃—Ph | —C(═O)CH₂CF₂H |
| Z-5782 | —C(═O)—c-Hex | —C(═O)CH₂CF₃ | Z-5783 | —C(═O)—Ph | —C(═O)CH₂CF₃ | Z-5784 | —C(═O)—4-OCF₃—Ph | —C(═O)CH₂CF₃ |
| Z-5785 | —C(═O)—c-Hex | —C(═O)CH₂CH₂CF₂H | Z-5786 | —C(═O)—Ph | —C(═O)CH₂CH₂CF₂H | Z-5787 | —C(═O)—4-OCF₃—Ph | —C(═O)CH₂CH₂CF₂H |
| Z-5788 | —C(═O)—c-Hex | —C(═O)CH₂CH₂CF₃ | Z-5789 | —C(═O)—Ph | —C(═O)CH₂CH₂CF₃ | Z-5790 | —C(═O)—4-OCF₃—Ph | —C(═O)CH₂CH₂CF₃ |
| Z-5791 | —C(═O)—c-Hex | —C(═O)CH₂CH₂CH₂CF₂H | Z-5792 | —C(═O)—Ph | —C(═O)CH₂CH₂CH₂CF₂H | Z-5793 | —C(═O)—4-OCF₃—Ph | —C(═O)CH₂CH₂CH₂CF₂H |
| Z-5794 | —C(═O)—c-Hex | —C(═O)CH₂CH₂CH₂CF₃ | Z-5795 | —C(═O)—Ph | —C(═O)CH₂CH₂CH₂CF₃ | Z-5796 | —C(═O)—4-OCF₃—Ph | —C(═O)CH₂CH₂CH₂CF₃ |
| Z-5797 | —C(═O)—c-Hex | —C(═O)CF₂CH₃ | Z-5798 | —C(═O)—Ph | —C(═O)CF₂CH₃ | Z-5799 | —C(═O)—4-OCF₃—Ph | —C(═O)CF₂CH₃ |
| Z-5800 | —C(═O)—c-Hex | —C(═O)CF₂CF₂H | Z-5801 | —C(═O)—Ph | —C(═O)CF₂CF₂H | Z-5802 | —C(═O)—4-OCF₃—Ph | —C(═O)CF₂CF₂H |
| Z-5803 | —C(═O)—c-Hex | —C(═O)CF₂CF₃ | Z-5804 | —C(═O)—Ph | —C(═O)CF₂CF₃ | Z-5805 | —C(═O)—4-OCF₃—Ph | —C(═O)CF₂CF₃ |
| Z-5806 | —C(═O)—c-Hex | —C(═O)CF₂CClF₂ | Z-5807 | —C(═O)—Ph | —C(═O)CF₂CClF₂ | Z-5808 | —C(═O)—4-OCF₃—Ph | —C(═O)CF₂CClF₂ |
| Z-5809 | —C(═O)—c-Hex | —C(═O)CFHCF₃ | Z-5810 | —C(═O)—Ph | —C(═O)CFHCF₃ | Z-5811 | —C(═O)—4-OCF₃—Ph | —C(═O)CFHCF₃ |
| Z-5812 | —C(═O)—c-Hex | —C(═O)CH₂CF₂CF₂H | Z-5813 | —C(═O)—Ph | —C(═O)CH₂CF₂CF₂H | Z-5814 | —C(═O)—4-OCF₃—Ph | —C(═O)CH₂CF₂CF₂H |
| Z-5815 | —C(═O)—c-Hex | —C(═O)CH₂CF₂CF₃ | Z-5816 | —C(═O)—Ph | —C(═O)CH₂CF₂CF₃ | Z-5817 | —C(═O)—4-OCF₃—Ph | —C(═O)CH₂CF₂CF₃ |
| Z-5818 | —C(═O)—c-Hex | —C(═O)CF₂CF₂CF₃ | Z-5819 | —C(═O)—Ph | —C(═O)CF₂CF₂CF₃ | Z-5820 | —C(═O)—4-OCF₃—Ph | —C(═O)CF₂CF₂CF₃ |
| Z-5821 | —C(═O)—c-Hex | —C(═O)CH₂CF₂CF₂CF₃ | Z-5822 | —C(═O)—Ph | —C(═O)CH₂CF₂CF₂CF₃ | Z-5823 | —C(═O)—4-OCF₃—Ph | —C(═O)CH₂CF₂CF₂CF₃ |
| Z-5824 | —C(═O)—c-Hex | —C(═O)CF₂CF₂CF₂CF₃ | Z-5825 | —C(═O)—Ph | —C(═O)CF₂CF₂CF₂CF₃ | Z-5826 | —C(═O)—4-OCF₃—Ph | —C(═O)CF₂CF₂CF₂CF₃ |
| Z-5827 | —C(═O)—c-Hex | —C(═O)CH₂CF₂CF₂CF₂CF₃ | Z-5828 | —C(═O)—Ph | —C(═O)CH₂CF₂CF₂CF₂CF₃ | Z-5829 | —C(═O)—4-OCF₃—Ph | —C(═O)CH₂CF₂CF₂CF₂CF₃ |
| Z-5830 | —C(═O)—c-Hex | —C(═O)CF₂CF₂CF₂CF₂CF₃ | Z-5831 | —C(═O)—Ph | —C(═O)CF₂CF₂CF₂CF₂CF₃ | Z-5832 | —C(═O)—4-OCF₃—Ph | —C(═O)CF₂CF₂CF₂CF₂CF₃ |
| Z-5833 | —C(═O)—c-Hex | —C(═O)—c-Pr | Z-5834 | —C(═O)—Ph | —C(═O)—c-Pr | Z-5835 | —C(═O)—4-OCF₃—Ph | —C(═O)—c-Pr |
| Z-5836 | —C(═O)—c-Hex | —C(═O)—c-Bu | Z-5837 | —C(═O)—Ph | —C(═O)—c-Bu | Z-5838 | —C(═O)—4-OCF₃—Ph | —C(═O)—c-Bu |
| Z-5839 | —C(═O)—c-Hex | —C(═O)—c-Pent | Z-5840 | —C(═O)—Ph | —C(═O)—c-Pent | Z-5841 | —C(═O)—4-OCF₃—Ph | —C(═O)—c-Pent |
| Z-5842 | —C(═O)—c-Hex | —C(═O)—c-Hex | Z-5843 | —C(═O)—Ph | —C(═O)—c-Hex | Z-5844 | —C(═O)—4-OCF₃—Ph | —C(═O)—c-Hex |
| Z-5845 | —C(═O)—c-Hex | —C(═O)—c-Hept | Z-5846 | —C(═O)—Ph | —C(═O)—c-Hept | Z-5847 | —C(═O)—4-OCF₃—Ph | —C(═O)—c-Hept |
| Z-5848 | —C(═O)—c-Hex | —C(═O)—c-Oct | Z-5849 | —C(═O)—Ph | —C(═O)—c-Oct | Z-5850 | —C(═O)—4-OCF₃—Ph | —C(═O)—c-Oct |
| Z-5851 | —C(═O)—c-Hex | —C(═O)CH═CH₂ | Z-5852 | —C(═O)—Ph | —C(═O)CH═CH₂ | Z-5853 | —C(═O)—4-OCF₃—Ph | —C(═O)CH═CH₂ |
| Z-5854 | —C(═O)—c-Hex | —C(═O)CH₂CH═CH₂ | Z-5855 | —C(═O)—Ph | —C(═O)CH₂CH═CH₂ | Z-5856 | —C(═O)—4-OCF₃—Ph | —C(═O)CH₂CH═CH₂ |
| Z-5857 | —C(═O)—c-Hex | —C(═O)CH═CHCH₃ | Z-5858 | —C(═O)—Ph | —C(═O)CH═CHCH₃ | Z-5859 | —C(═O)—4-OCF₃—Ph | —C(═O)CH═CHCH₃ |
| Z-5860 | —C(═O)—c-Hex | —C(═O)CH₂C(CH₃)═CH₂ | Z-5861 | —C(═O)—Ph | —C(═O)CH₂C(CH₃)═CH₂ | Z-5862 | —C(═O)—4-OCF₃—Ph | —C(═O)CH₂C(CH₃)═CH₂ |
| Z-5863 | —C(═O)—c-Hex | —C(═O)CH₂CH₂CH═CH₂ | Z-5864 | —C(═O)—Ph | —C(═O)CH₂CH₂CH═CH₂ | Z-5865 | —C(═O)—4-OCF₃—Ph | —C(═O)CH₂CH₂CH═CH₂ |
| Z-5866 | —C(═O)—c-Hex | —C(═O)CH₂CH═CHCH₃ | Z-5867 | —C(═O)—Ph | —C(═O)CH₂CH═CHCH₃ | Z-5868 | —C(═O)—4-OCF₃—Ph | —C(═O)CH₂CH═CHCH₃ |
| Z-5869 | —C(═O)—c-Hex | —C(═O)CH═CHCH₂CH₃ | Z-5870 | —C(═O)—Ph | —C(═O)CH═CHCH₂CH₃ | Z-5871 | —C(═O)—4-OCF₃—Ph | —C(═O)CH═CHCH₂CH₃ |
| Z-5872 | —C(═O)—c-Hex | —C(═O)CH₂CH═C(CH₃)₂ | Z-5873 | —C(═O)—Ph | —C(═O)CH₂CH═C(CH₃)₂ | Z-5874 | —C(═O)—4-OCF₃—Ph | —C(═O)CH₂CH═C(CH₃)₂ |
| Z-5875 | —C(═O)—c-Hex | —C(═O)CH₂CH₂CH═C(CH₃)₂ | Z-5876 | —C(═O)—Ph | —C(═O)CH₂CH₂CH═C(CH₃)₂ | Z-5877 | —C(═O)—4-OCF₃—Ph | —C(═O)CH₂CH₂CH═C(CH₃)₂ |
| Z-5878 | —C(═O)—c-Hex | —C(═O)CH═CFH | Z-5879 | —C(═O)—Ph | —C(═O)CH═CFH | Z-5880 | —C(═O)—4-OCF₃—Ph | —C(═O)CH═CFH |
| Z-5881 | —C(═O)—c-Hex | —C(═O)CH═CF₂ | Z-5882 | —C(═O)—Ph | —C(═O)CH═CF₂ | Z-5883 | —C(═O)—4-OCF₃—Ph | —C(═O)CH═CF₂ |
| Z-5884 | —C(═O)—c-Hex | —C(═O)CH═CCl₂ | Z-5885 | —C(═O)—Ph | —C(═O)CH═CCl₂ | Z-5886 | —C(═O)—4-OCF₃—Ph | —C(═O)CH═CCl₂ |
| Z-5887 | —C(═O)—c-Hex | —C(═O)CH₂CH═CFH | Z-5888 | —C(═O)—Ph | —C(═O)CH₂CH═CFH | Z-5889 | —C(═O)—4-OCF₃—Ph | —C(═O)CH₂CH═CFH |
| Z-5890 | —C(═O)—c-Hex | —C(═O)CH₂CH═CF₂ | Z-5891 | —C(═O)—Ph | —C(═O)CH₂CH═CF₂ | Z-5892 | —C(═O)—4-OCF₃—Ph | —C(═O)CH₂CH═CF₂ |
| Z-5893 | —C(═O)—c-Hex | —C(═O)CH₂CH═CCl₂ | Z-5894 | —C(═O)—Ph | —C(═O)CH₂CH═CCl₂ | Z-5895 | —C(═O)—4-OCF₃—Ph | —C(═O)CH₂CH═CCl₂ |
| Z-5896 | —C(═O)—c-Hex | —C(═O)CH₂CH₂CH═CF₂ | Z-5897 | —C(═O)—Ph | —C(═O)CH₂CH₂CH═CF₂ | Z-5898 | —C(═O)—4-OCF₃—Ph | —C(═O)CH₂CH₂CH═CF₂ |
| Z-5899 | —C(═O)—c-Hex | —C(═O)CH₂CH₂CH₂CH═CF₂ | Z-5900 | —C(═O)—Ph | —C(═O)CH₂CH₂CH₂CH═CF₂ | Z-5901 | —C(═O)—4-OCF₃—Ph | —C(═O)CH₂CH₂CH₂CH═CF₂ |
| Z-5902 | —C(═O)—c-Hex | —C(═O)CH₂CH₂CH₂CH₂CH═CF₂ | Z-5903 | —C(═O)—Ph | —C(═O)CH₂CH₂CH₂CH₂CH═CF₂ | Z-5904 | —C(═O)—4-OCF₃—Ph | —C(═O)CH₂CH₂CH₂CH₂CH═CF₂ |
| Z-5905 | —C(═O)—c-Hex | —C(═O)C≡CH | Z-5906 | —C(═O)—Ph | —C(═O)C≡CH | Z-5907 | —C(═O)—4-OCF₃—Ph | —C(═O)C≡CH |
| Z-5908 | —C(═O)—c-Hex | —C(═O)C≡CCH₃ | Z-5909 | —C(═O)—Ph | —C(═O)C≡CCH₃ | Z-5910 | —C(═O)—4-OCF₃—Ph | —C(═O)C≡CCH₃ |
| Z-5911 | —C(═O)—c-Hex | —C(═O)CH₂C≡CH | Z-5912 | —C(═O)—Ph | —C(═O)CH₂C≡CH | Z-5913 | —C(═O)—4-OCF₃—Ph | —C(═O)CH₂C≡CH |
| Z-5914 | —C(═O)—c-Hex | —C(═O)C≡CCH₂CH₃ | Z-5915 | —C(═O)—Ph | —C(═O)C≡CCH₂CH₃ | Z-5916 | —C(═O)—4-OCF₃—Ph | —C(═O)C≡CCH₂CH₃ |
| Z-5917 | —C(═O)—c-Hex | —C(═O)CH₂C≡CCH₃ | Z-5918 | —C(═O)—Ph | —C(═O)CH₂C≡CCH₃ | Z-5919 | —C(═O)—4-OCF₃—Ph | —C(═O)CH₂C≡CCH₃ |
| Z-5920 | —C(═O)—c-Hex | —C(═O)CH₂CH₂C≡CH | Z-5921 | —C(═O)—Ph | —C(═O)CH₂CH₂C≡CH | Z-5922 | —C(═O)—4-OCF₃—Ph | —C(═O)CH₂CH₂C≡CH |
| Z-5923 | —C(═O)—c-Hex | —C(═O)C≡CCH₂CH₂CH₃ | Z-5924 | —C(═O)—Ph | —C(═O)C≡CCH₂CH₂CH₃ | Z-5925 | —C(═O)—4-OCF₃—Ph | —C(═O)C≡CCH₂CH₂CH₃ |
| Z-5926 | —C(═O)—c-Hex | —C(═O)CH₂C≡CCH₂CH₃ | Z-5927 | —C(═O)—Ph | —C(═O)CH₂C≡CCH₂CH₃ | Z-5928 | —C(═O)—4-OCF₃—Ph | —C(═O)CH₂C≡CCH₂CH₃ |
| Z-5929 | —C(═O)—c-Hex | —C(═O)C(CH₃)₂C≡CH | Z-5930 | —C(═O)—Ph | —C(═O)C(CH₃)₂C≡CH | Z-5931 | —C(═O)—4-OCF₃—Ph | —C(═O)C(CH₃)₂C≡CH |
| Z-5932 | —C(═O)—c-Hex | —C(═O)C≡CF | Z-5933 | —C(═O)—Ph | —C(═O)C≡CF | Z-5934 | —C(═O)—4-OCF₃—Ph | —C(═O)C≡CF |
| Z-5935 | —C(═O)—c-Hex | —C(═O)C≡CCF₂H | Z-5936 | —C(═O)—Ph | —C(═O)C≡CCF₂H | Z-5937 | —C(═O)—4-OCF₃—Ph | —C(═O)C≡CCF₂H |
| Z-5938 | —C(═O)—c-Hex | —C(═O)C≡CCF₃ | Z-5939 | —C(═O)—Ph | —C(═O)C≡CCF₃ | Z-5940 | —C(═O)—4-OCF₃—Ph | —C(═O)C≡CCF₃ |
| Z-5941 | —C(═O)—c-Hex | —C(═O)C≡CCH₂CF₂H | Z-5942 | —C(═O)—Ph | —C(═O)C≡CCH₂CF₂H | Z-5943 | —C(═O)—4-OCF₃—Ph | —C(═O)C≡CCH₂CF₂H |
| Z-5944 | —C(═O)—c-Hex | —C(═O)C≡CCH₂CF₃ | Z-5945 | —C(═O)—Ph | —C(═O)C≡CCH₂CF₃ | Z-5946 | —C(═O)—4-OCF₃—Ph | —C(═O)C≡CCH₂CF₃ |
| Z-5947 | —C(═O)—c-Hex | —C(═O)CH₂C≡CHCF₂H | Z-5948 | —C(═O)—Ph | —C(═O)CH₂C≡CHCF₂H | Z-5949 | —C(═O)—4-OCF₃—Ph | —C(═O)CH₂C≡CHCF₂H |
| Z-5950 | —C(═O)—c-Hex | —C(═O)CH₂C≡CCF₃ | Z-5951 | —C(═O)—Ph | —C(═O)CH₂C≡CCF₃ | Z-5952 | —C(═O)—4-OCF₃—Ph | —C(═O)CH₂C≡CCF₃ |
| Z-5953 | —C(═O)—c-Hex | —C(═O)CH₂C≡N | Z-5954 | —C(═O)—Ph | —C(═O)CH₂C≡N | Z-5955 | —C(═O)—4-OCF₃—Ph | —C(═O)CH₂C≡N |
| Z-5956 | —C(═O)—c-Hex | —C(═O)C(Me)C≡N | Z-5957 | —C(═O)—Ph | —C(═O)C(Me)C≡N | Z-5958 | —C(═O)—4-OCF₃—Ph | —C(═O)C(Me)C≡N |
| Z-5959 | —C(═O)—c-Hex | —C(═O)CH₂CH₂C≡N | Z-5960 | —C(═O)—Ph | —C(═O)CH₂CH₂C≡N | Z-5961 | —C(═O)—4-OCF₃—Ph | —C(═O)CH₂CH₂C≡N |
| Z-5962 | —C(═O)—c-Hex | —C(═O)CH₂CH₂CH₂C≡N | Z-5963 | —C(═O)—Ph | —C(═O)CH₂CH₂CH₂C≡N | Z-5964 | —C(═O)—4-OCF₃—Ph | —C(═O)CH₂CH₂CH₂C≡N |
| Z-5965 | —C(═O)—c-Hex | —C(═O)CH₂OH | Z-5966 | —C(═O)—Ph | —C(═O)CH₂OH | Z-5967 | —C(═O)—4-OCF₃—Ph | —C(═O)CH₂OH |
| Z-5968 | —C(═O)—c-Hex | —C(═O)CH₂OMe | Z-5969 | —C(═O)—Ph | —C(═O)CH₂OMe | Z-5970 | —C(═O)—4-OCF₃—Ph | —C(═O)CH₂OMe |
| Z-5971 | —C(═O)—c-Hex | —C(═O)CH₂OEt | Z-5972 | —C(═O)—Ph | —C(═O)CH₂OEt | Z-5973 | —C(═O)—4-OCF₃—Ph | —C(═O)CH₂OEt |
| Z-5974 | —C(═O)—c-Hex | —C(═O)CH₂OPr | Z-5975 | —C(═O)—Ph | —C(═O)CH₂OPr | Z-5976 | —C(═O)—4-OCF₃—Ph | —C(═O)CH₂OPr |
| Z-5977 | —C(═O)—c-Hex | —C(═O)CH₂CH₂OMe | Z-5978 | —C(═O)—Ph | —C(═O)CH₂CH₂OMe | Z-5979 | —C(═O)—4-OCF₃—Ph | —C(═O)CH₂CH₂OMe |
| Z-5980 | —C(═O)—c-Hex | —C(═O)CH₂CH₂OEt | Z-5981 | —C(═O)—Ph | —C(═O)CH₂CH₂OEt | Z-5982 | —C(═O)—4-OCF₃—Ph | —C(═O)CH₂CH₂OEt |
| Z-5983 | —C(═O)—c-Hex | —C(═O)CH₂—(1-Pyra) | Z-5984 | —C(═O)—Ph | —C(═O)CH₂—(1-Pyra) | Z-5985 | —C(═O)—4-OCF₃—Ph | —C(═O)CH₂—(1-Pyra) |
| Z-5986 | —C(═O)—c-Hex | —C(═O)CH₂—(1-Tria) | Z-5987 | —C(═O)—Ph | —C(═O)CH₂—(1-Tria) | Z-5988 | —C(═O)—4-OCF₃—Ph | —C(═O)CH₂—(1-Tria) |
| Z-5989 | —C(═O)—c-Hex | —C(═O)OH | Z-5990 | —C(═O)—Ph | —C(═O)OH | Z-5991 | —C(═O)—4-OCF₃—Ph | —C(═O)OH |
| Z-5992 | —C(═O)—c-Hex | —C(═O)OMe | Z-5993 | —C(═O)—Ph | —C(═O)OMe | Z-5994 | —C(═O)—4-OCF₃—Ph | —C(═O)OMe |
| Z-5995 | —C(═O)—c-Hex | —C(═O)OEt | Z-5996 | —C(═O)—Ph | —C(═O)OEt | Z-5997 | —C(═O)—4-OCF₃—Ph | —C(═O)OEt |
| Z-5998 | —C(═O)—c-Hex | —C(═O)OPr | Z-5999 | —C(═O)—Ph | —C(═O)OPr | Z-6000 | —C(═O)—4-OCF₃—Ph | —C(═O)OPr |
| Z-6001 | —C(═O)—c-Hex | —C(═O)O—i-Pr | Z-6002 | —C(═O)—Ph | —C(═O)O—i-Pr | Z-6003 | —C(═O)—4-OCF₃—Ph | —C(═O)O—i-Pr |
| Z-6004 | —C(═O)—c-Hex | —C(═O)OBu | Z-6005 | —C(═O)—Ph | —C(═O)OBu | Z-6006 | —C(═O)—4-OCF₃—Ph | —C(═O)OBu |
| Z-6007 | —C(═O)—c-Hex | —C(═O)O—sec-Bu | Z-6008 | —C(═O)—Ph | —C(═O)O—sec-Bu | Z-6009 | —C(═O)—4-OCF₃—Ph | —C(═O)O—sec-Bu |
| Z-6010 | —C(═O)—c-Hex | —C(═O)O—i-Bu | Z-6011 | —C(═O)—Ph | —C(═O)O—i-Bu | Z-6012 | —C(═O)—4-OCF₃—Ph | —C(═O)O—i-Bu |
| Z-6013 | —C(═O)—c-Hex | —C(═O)O—t-Bu | Z-6014 | —C(═O)—Ph | —C(═O)O—t-Bu | Z-6015 | —C(═O)—4-OCF₃—Ph | —C(═O)O—t-Bu |
| Z-6016 | —C(═O)—c-Hex | —C(═O)OPent | Z-6017 | —C(═O)—Ph | —C(═O)OPent | Z-6018 | —C(═O)—4-OCF₃—Ph | —C(═O)OPent |
| Z-6019 | —C(═O)—c-Hex | —C(═O)OHex | Z-6020 | —C(═O)—Ph | —C(═O)OHex | Z-6021 | —C(═O)—4-OCF₃—Ph | —C(═O)OHex |
| Z-6022 | —C(═O)—c-Hex | —C(═O)OCH(CH₃)CH₂CH₂CH₃ | Z-6023 | —C(═O)—Ph | —C(═O)OCH(CH₃)CH₂CH₂CH₃ | Z-6024 | —C(═O)—4-OCF₃—Ph | —C(═O)OCH(CH₃)CH₂CH₂CH₃ |
| Z-6025 | —C(═O)—c-Hex | —C(═O)OCH(CH₃)CH(CH₃)₂ | Z-6026 | —C(═O)—Ph | —C(═O)OCH(CH₃)CH(CH₃)₂ | Z-6027 | —C(═O)—4-OCF₃—Ph | —C(═O)OCH(CH₃)CH(CH₃)₂ |
| Z-6028 | —C(═O)—c-Hex | —C(═O)OC(CH₃)₂CH₂CH₃ | Z-6029 | —C(═O)—Ph | —C(═O)OC(CH₃)₂CH₂CH₃ | Z-6030 | —C(═O)—4-OCF₃—Ph | —C(═O)OC(CH₃)₂CH₂CH₃ |
| Z-6031 | —C(═O)—c-Hex | —C(═O)OCH(CH₂CH₃)₂ | Z-6032 | —C(═O)—Ph | —C(═O)OCH(CH₂CH₃)₂ | Z-6033 | —C(═O)—4-OCF₃—Ph | —C(═O)OCH(CH₂CH₃)₂ |
| Z-6034 | —C(═O)—c-Hex | —C(═O)OCH₂CH₂CH(CH₃)₂ | Z-6035 | —C(═O)—Ph | —C(═O)OCH₂CH₂CH(CH₃)₂ | Z-6036 | —C(═O)—4-OCF₃—Ph | —C(═O)OCH₂CH₂CH(CH₃)₂ |
| Z-6037 | —C(═O)—c-Hex | —C(═O)OCH₂CH₂CH₂CH(CH₃)₂ | Z-6038 | —C(═O)—Ph | —C(═O)OCH₂CH₂CH₂CH(CH₃)₂ | Z-6039 | —C(═O)—4-OCF₃—Ph | —C(═O)OCH₂CH₂CH₂CH(CH₃)₂ |
| Z-6040 | —C(═O)—c-Hex | —C(═O)OCH₂CH₂CH(CH₃)CH₂CH₃ | Z-6041 | —C(═O)—Ph | —C(═O)OCH₂CH₂CH(CH₃)CH₂CH₃ | Z-6042 | —C(═O)—4-OCF₃—Ph | —C(═O)OCH₂CH₂CH(CH₃)CH₂CH₃ |
| Z-6043 | —C(═O)—c-Hex | —C(═O)OCH₂CH(CH₃)CH₂CH₂CH₃ | Z-6044 | —C(═O)—Ph | —C(═O)OCH₂CH(CH₃)CH₂CH₂CH₃ | Z-6045 | —C(═O)—4-OCF₃—Ph | —C(═O)OCH₂CH(CH₃)CH₂CH₂CH₃ |
| Z-6046 | —C(═O)—c-Hex | —C(═O)OCH(CH₃)CH₂CH₂CH₂CH₃ | Z-6047 | —C(═O)—Ph | —C(═O)OCH(CH₃)CH₂CH₂CH₂CH₃ | Z-6048 | —C(═O)—4-OCF₃—Ph | —C(═O)OCH(CH₃)CH₂CH₂CH₂CH₃ |
| Z-6049 | —C(═O)—c-Hex | —C(═O)OCH(CH₃)CH₂CH(CH₃)₂ | Z-6050 | —C(═O)—Ph | —C(═O)OCH(CH₃)CH₂CH(CH₃)₂ | Z-6051 | —C(═O)—4-OCF₃—Ph | —C(═O)OCH(CH₃)CH₂CH(CH₃)₂ |
| Z-6052 | —C(═O)—c-Hex | —C(═O)OCH(CH₃)CH(CH₃)CH₂CH₃ | Z-6053 | —C(═O)—Ph | —C(═O)OCH(CH₃)CH(CH₃)CH₂CH₃ | Z-6054 | —C(═O)—4-OCF₃—Ph | —C(═O)OCH(CH₃)CH(CH₃)CH₂CH₃ |
| Z-6055 | —C(═O)—c-Hex | —C(═O)OC(CH₃)₂CH₂CH₂CH₃ | Z-6056 | —C(═O)—Ph | —C(═O)OC(CH₃)₂CH₂CH₂CH₃ | Z-6057 | —C(═O)—4-OCF₃—Ph | —C(═O)OC(CH₃)₂CH₂CH₂CH₃ |
| Z-6058 | —C(═O)—c-Hex | —C(═O)OCH(CH₃)C(CH₃)₃ | Z-6059 | —C(═O)—Ph | —C(═O)OCH(CH₃)C(CH₃)₃ | Z-6060 | —C(═O)—4-OCF₃—Ph | —C(═O)OCH(CH₃)C(CH₃)₃ |
| Z-6061 | —C(═O)—c-Hex | —C(═O)OC(CH₃)₂CH(CH₃)₂ | Z-6062 | —C(═O)—Ph | —C(═O)OC(CH₃)₂CH(CH₃)₂ | Z-6063 | —C(═O)—4-OCF₃—Ph | —C(═O)OC(CH₃)₂CH(CH₃)₂ |
| Z-6064 | —C(═O)—c-Hex | —C(═O)OCH₂CH₂C(CH₃)₃ | Z-6065 | —C(═O)—Ph | —C(═O)OCH₂CH₂C(CH₃)₃ | Z-6066 | —C(═O)—4-OCF₃—Ph | —C(═O)OCH₂CH₂C(CH₃)₃ |
| Z-6067 | —C(═O)—c-Hex | —C(═O)OCH₂CH(CH₃)CH(CH₃)₂ | Z-6068 | —C(═O)—Ph | —C(═O)OCH₂CH(CH₃)CH(CH₃)₂ | Z-6069 | —C(═O)—4-OCF₃—Ph | —C(═O)OCH₂CH(CH₃)CH(CH₃)₂ |
| Z-6070 | —C(═O)—c-Hex | —C(═O)OCH₂C(CH₃)₂CH₂CH₃ | Z-6071 | —C(═O)—Ph | —C(═O)OCH₂C(CH₃)₂CH₂CH₃ | Z-6072 | —C(═O)—4-OCF₃—Ph | —C(═O)OCH₂C(CH₃)₂CH₂CH₃ |
| Z-6073 | —C(═O)—c-Hex | —C(═O)OCFH₂ | Z-6074 | —C(═O)—Ph | —C(═O)OCFH₂ | Z-6075 | —C(═O)—4-OCF₃—Ph | —C(═O)OCFH₂ |
| Z-6076 | —C(═O)—c-Hex | —C(═O)OCF₂H | Z-6077 | —C(═O)—Ph | —C(═O)OCF₂H | Z-6078 | —C(═O)—4-OCF₃—Ph | —C(═O)OCF₂H |
| Z-6079 | —C(═O)—c-Hex | —C(═O)OCF₃ | Z-6080 | —C(═O)—Ph | —C(═O)OCF₃ | Z-6081 | —C(═O)—4-OCF₃—Ph | —C(═O)OCF₃ |
| Z-6082 | —C(═O)—c-Hex | —C(═O)OCH₂Cl | Z-6083 | —C(═O)—Ph | —C(═O)OCH₂Cl | Z-6084 | —C(═O)—4-OCF₃—Ph | —C(═O)OCH₂Cl |
| Z-6085 | —C(═O)—c-Hex | —C(═O)OCHCl₂ | Z-6086 | —C(═O)—Ph | —C(═O)OCHCl₂ | Z-6087 | —C(═O)—4-OCF₃—Ph | —C(═O)OCHCl₂ |
| Z-6088 | —C(═O)—c-Hex | —C(═O)OCCl₃ | Z-6089 | —C(═O)—Ph | —C(═O)OCCl₃ | Z-6090 | —C(═O)—4-OCF₃—Ph | —C(═O)OCCl₃ |
| Z-6091 | —C(═O)—c-Hex | —C(═O)OCH₂Br | Z-6092 | —C(═O)—Ph | —C(═O)OCH₂Br | Z-6093 | —C(═O)—4-OCF₃—Ph | —C(═O)OCH₂Br |
| Z-6094 | —C(═O)—c-Hex | —C(═O)OCHBr₂ | Z-6095 | —C(═O)—Ph | —C(═O)OCHBr₂ | Z-6096 | —C(═O)—4-OCF₃—Ph | —C(═O)OCHBr₂ |
| Z-6097 | —C(═O)—c-Hex | —C(═O)OCBr₃ | Z-6098 | —C(═O)—Ph | —C(═O)OCBr₃ | Z-6099 | —C(═O)—4-OCF₃—Ph | —C(═O)OCBr₃ |
| Z-6100 | —C(═O)—c-Hex | —C(═O)OCH₂l | Z-6101 | —C(═O)—Ph | —C(═O)OCH₂l | Z-6102 | —C(═O)—4-OCF₃—Ph | —C(═O)OCH₂l |
| Z-6103 | —C(═O)—c-Hex | —C(═O)OCHl₂ | Z-6104 | —C(═O)—Ph | —C(═O)OCHl₂ | Z-6105 | —C(═O)—4-OCF₃—Ph | —C(═O)OCHl₂ |
| Z-6106 | —C(═O)—c-Hex | —C(═O)OCH₂CF₂H | Z-6107 | —C(═O)—Ph | —C(═O)OCH₂CF₂H | Z-6108 | —C(═O)—4-OCF₃—Ph | —C(═O)OCH₂CF₂H |
| Z-6109 | —C(═O)—c-Hex | —C(═O)OCH₂CF₃ | Z-6110 | —C(═O)—Ph | —C(═O)OCH₂CF₃ | Z-6111 | —C(═O)—4-OCF₃—Ph | —C(═O)OCH₂CF₃ |
| Z-6112 | —C(═O)—c-Hex | —C(═O)OCH₂CH₂CF₂H | Z-6113 | —C(═O)—Ph | —C(═O)OCH₂CH₂CF₂H | Z-6114 | —C(═O)—4-OCF₃—Ph | —C(═O)OCH₂CH₂CF₂H |
| Z-6115 | —C(═O)—c-Hex | —C(═O)OCH₂CH₂CF₃ | Z-6116 | —C(═O)—Ph | —C(═O)OCH₂CH₂CF₃ | Z-6117 | —C(═O)—4-OCF₃—Ph | —C(═O)OCH₂CH₂CF₃ |
| Z-6118 | —C(═O)—c-Hex | —C(═O)OCH₂CH₂CH₂CF₂H | Z-6119 | —C(═O)—Ph | —C(═O)OCH₂CH₂CH₂CF₂H | Z-6120 | —C(═O)—4-OCF₃—Ph | —C(═O)OCH₂CH₂CH₂CF₂H |
| Z-6121 | —C(═O)—c-Hex | —C(═O)OCH₂CH₂CH₂CF₃ | Z-6122 | —C(═O)—Ph | —C(═O)OCH₂CH₂CH₂CF₃ | Z-6123 | —C(═O)—4-OCF₃—Ph | —C(═O)OCH₂CH₂CH₂CF₃ |
| Z-6124 | —C(═O)—c-Hex | —C(═O)OCF₂CF₂H | Z-6125 | —C(═O)—Ph | —C(═O)OCF₂CF₂H | Z-6126 | —C(═O)—4-OCF₃—Ph | —C(═O)OCF₂CF₂H |
| Z-6127 | —C(═O)—c-Hex | —C(═O)OCF₂CF₃ | Z-6128 | —C(═O)—Ph | —C(═O)OCF₂CF₃ | Z-6129 | —C(═O)—4-OCF₃—Ph | —C(═O)OCF₂CF₃ |
| Z-6130 | —C(═O)—c-Hex | —C(═O)OCFHCF₃ | Z-6131 | —C(═O)—Ph | —C(═O)OCFHCF₃ | Z-6132 | —C(═O)—4-OCF₃—Ph | —C(═O)OCFHCF₃ |
| Z-6133 | —C(═O)—c-Hex | —C(═O)OCH₂CF₂CF₂H | Z-6134 | —C(═O)—Ph | —C(═O)OCH₂CF₂CF₂H | Z-6135 | —C(═O)—4-OCF₃—Ph | —C(═O)OCH₂CF₂CF₂H |
| Z-6136 | —C(═O)—c-Hex | —C(═O)OCH₂CF₂CF₃ | Z-6137 | —C(═O)—Ph | —C(═O)OCH₂CF₂CF₃ | Z-6138 | —C(═O)—4-OCF₃—Ph | —C(═O)OCH₂CF₂CF₃ |
| Z-6139 | —C(═O)—c-Hex | —C(═O)OCF₂CF₂CF₃ | Z-6140 | —C(═O)—Ph | —C(═O)OCF₂CF₂CF₃ | Z-6141 | —C(═O)—4-OCF₃—Ph | —C(═O)OCF₂CF₂CF₃ |
| Z-6142 | —C(═O)—c-Hex | —C(═O)OCH₂CF₂CF₂CF₃ | Z-6143 | —C(═O)—Ph | —C(═O)OCH₂CF₂CF₂CF₃ | Z-6144 | —C(═O)—4-OCF₃—Ph | —C(═O)OCH₂CF₂CF₂CF₃ |
| Z-6145 | —C(═O)—c-Hex | —C(═O)OCF₂CF₂CF₂CF₃ | Z-6146 | —C(═O)—Ph | —C(═O)OCF₂CF₂CF₂CF₃ | Z-6147 | —C(═O)—4-OCF₃—Ph | —C(═O)OCF₂CF₂CF₂CF₃ |
| Z-6148 | —C(═O)—c-Hex | —C(═O)OCH₂CF₂CF₂CF₂CF₃ | Z-6149 | —C(═O)—Ph | —C(═O)OCH₂CF₂CF₂CF₂CF₃ | Z-6150 | —C(═O)—4-OCF₃—Ph | —C(═O)OCH₂CF₂CF₂CF₂CF₃ |
| Z-6151 | —C(═O)—c-Hex | —C(═O)O—c-Pr | Z-6152 | —C(═O)—Ph | —C(═O)O—c-Pr | Z-6153 | —C(═O)—4-OCF₃—Ph | —C(═O)O—c-Pr |
| Z-6154 | —C(═O)—c-Hex | —C(═O)O—c-Bu | Z-6155 | —C(═O)—Ph | —C(═O)O—c-Bu | Z-6156 | —C(═O)—4-OCF₃—Ph | —C(═O)O—c-Bu |
| Z-6157 | —C(═O)—c-Hex | —C(═O)O—c-Pent | Z-6158 | —C(═O)—Ph | —C(═O)O—c-Pent | Z-6159 | —C(═O)—4-OCF₃—Ph | —C(═O)O—c-Pent |
| Z-6160 | —C(═O)—c-Hex | —C(═O)O—c-Hex | Z-6161 | —C(═O)—Ph | —C(═O)O—c-Hex | Z-6162 | —C(═O)—4-OCF₃—Ph | —C(═O)O—c-Hex |
| Z-6163 | —C(═O)—c-Hex | —C(═O)O—c-Hept | Z-6164 | —C(═O)—Ph | —C(═O)O—c-Hept | Z-6165 | —C(═O)—4-OCF₃—Ph | —C(═O)O—c-Hept |
| Z-6166 | —C(═O)—c-Hex | —C(═O)O—c-Oct | Z-6167 | —C(═O)—Ph | —C(═O)O—c-Oct | Z-6168 | —C(═O)—4-OCF₃—Ph | —C(═O)O—c-Oct |
| Z-6169 | —C(═O)—c-Hex | —C(═O)OCH═CH₂ | Z-6170 | —C(═O)—Ph | —C(═O)OCH═CH₂ | Z-6171 | —C(═O)—4-OCF₃—Ph | —C(═O)OCH═CH₂ |
| Z-6172 | —C(═O)—c-Hex | —C(═O)OCH₂CH═CH₂ | Z-6173 | —C(═O)—Ph | —C(═O)OCH₂CH═CH₂ | Z-6174 | —C(═O)—4-OCF₃—Ph | —C(═O)OCH₂CH═CH₂ |
| Z-6175 | —C(═O)—c-Hex | —C(═O)OCH═CHCH₃ | Z-6176 | —C(═O)—Ph | —C(═O)OCH═CHCH₃ | Z-6177 | —C(═O)—4-OCF₃—Ph | —C(═O)OCH═CHCH₃ |
| Z-6178 | —C(═O)—c-Hex | —C(═O)OCH₂C(CH₃)═CH₂ | Z-6179 | —C(═O)—Ph | —C(═O)OCH₂C(CH₃)═CH₂ | Z-6180 | —C(═O)—4-OCF₃—Ph | —C(═O)OCH₂C(CH₃)═CH₂ |
| Z-6181 | —C(═O)—c-Hex | —C(═O)OCH₂CH₂CH═CH₂ | Z-6182 | —C(═O)—Ph | —C(═O)OCH₂CH₂CH═CH₂ | Z-6183 | —C(═O)—4-OCF₃—Ph | —C(═O)OCH₂CH₂CH═CH₂ |
| Z-6184 | —C(═O)—c-Hex | —C(═O)OCH₂CH═CHCH₃ | Z-6185 | —C(═O)—Ph | —C(═O)OCH₂CH═CHCH₃ | Z-6186 | —C(═O)—4-OCF₃—Ph | —C(═O)OCH₂CH═CHCH₃ |
| Z-6187 | —C(═O)—c-Hex | —C(═O)OCH═CHCH₂CH₃ | Z-6188 | —C(═O)—Ph | —C(═O)OCH═CHCH₂CH₃ | Z-6189 | —C(═O)—4-OCF₃—Ph | —C(═O)OCH═CHCH₂CH₃ |
| Z-6190 | —C(═O)—c-Hex | —C(═O)OCH₂CH═C(CH₃)₂ | Z-6191 | —C(═O)—Ph | —C(═O)OCH₂CH═C(CH₃)₂ | Z-6192 | —C(═O)—4-OCF₃—Ph | —C(═O)OCH₂CH═C(CH₃)₂ |
| Z-6193 | —C(═O)—c-Hex | —C(═O)OCH2CH₂CH═C(CH₃)₂ | Z-6194 | —C(═O)—Ph | —C(═O)OCH2CH₂CH═C(CH₃)₂ | Z-6195 | —C(═O)—4-OCF₃—Ph | —C(═O)OCH2CH₂CH═C(CH₃)₂ |
| Z-6196 | —C(═O)—c-Hex | —C(═O)OCH═CFH | Z-6197 | —C(═O)—Ph | —C(═O)OCH═CFH | Z-6198 | —C(═O)—4-OCF₃—Ph | —C(═O)OCH═CFH |
| Z-6199 | —C(═O)—c-Hex | —C(═O)OCH═CF₂ | Z-6200 | —C(═O)—Ph | —C(═O)OCH═CF₂ | Z-6201 | —C(═O)—4-OCF₃—Ph | —C(═O)OCH═CF₂ |
| Z-6202 | —C(═O)—c-Hex | —C(═O)OCH═CCl₂ | Z-6203 | —C(═O)—Ph | —C(═O)OCH═CCl₂ | Z-6204 | —C(═O)—4-OCF₃—Ph | —C(═O)OCH═CCl₂ |
| Z-6205 | —C(═O)—c-Hex | —C(═O)OCH₂CH═CFH | Z-6206 | —C(═O)—Ph | —C(═O)OCH₂CH═CFH | Z-6207 | —C(═O)—4-OCF₃—Ph | —C(═O)OCH₂CH═CFH |
| Z-6208 | —C(═O)—c-Hex | —C(═O)OCH₂CH═CF₂ | Z-6209 | —C(═O)—Ph | —C(═O)OCH₂CH═CF₂ | Z-6210 | —C(═O)—4-OCF₃—Ph | —C(═O)OCH₂CH═CF₂ |
| Z-6211 | —C(═O)—c-Hex | —C(═O)OCH₂CH═CCl₂ | Z-6212 | —C(═O)—Ph | —C(═O)OCH₂CH═CCl₂ | Z-6213 | —C(═O)—4-OCF₃—Ph | —C(═O)OCH₂CH═CCl₂ |
| Z-6214 | —C(═O)—c-Hex | —C(═O)OCH₂CH₂CH═CF₂ | Z-6215 | —C(═O)—Ph | —C(═O)OCH₂CH₂CH═CF₂ | Z-6216 | —C(═O)—4-OCF₃—Ph | —C(═O)OCH₂CH₂CH═CF₂ |
| Z-6217 | —C(═O)—c-Hex | —C(═O)OCH₂CH₂CH₂CH═CF₂ | Z-6218 | —C(═O)—Ph | —C(═O)OCH₂CH₂CH₂CH═CF₂ | Z-6219 | —C(═O)—4-OCF₃—Ph | —C(═O)OCH₂CH₂CH₂CH═CF₂ |
| Z-6220 | —C(═O)—c-Hex | —C(═O)OCH₂CH₂CH₂CH₂CH═CF₂ | Z-6221 | —C(═O)—Ph | —C(═O)OCH₂CH₂CH₂CH₂CH═CF₂ | Z-6222 | —C(═O)—4-OCF₃—Ph | —C(═O)OCH₂CH₂CH₂CH₂CH═CF₂ |
| Z-6223 | —C(═O)—c-Hex | —C(═Q)OCH₂C≡CH | Z-6224 | —C(═O)—Ph | —C(═Q)OCH₂C≡CH | Z-6225 | —C(═O)—4-OCF₃—Ph | —C(═Q)OCH₂C≡CH |
| Z-6226 | —C(═O)—c-Hex | —C(═O)OCH₂C≡CCH₃ | Z-6227 | —C(═O)—Ph | —C(═O)OCH₂C≡CCH₃ | Z-6228 | —C(═O)—4-OCF₃—Ph | —C(═O)OCH₂C≡CCH₃ |
| Z-6229 | —C(═O)—c-Hex | —C(═O)OCH₂CH₂C≡CH | Z-6230 | —C(═O)—Ph | —C(═O)OCH₂CH₂C≡CH | Z-6231 | —C(═O)—4-OCF₃—Ph | —C(═O)OCH₂CH₂C≡CH |
| Z-6232 | —C(═O)—c-Hex | —C(═O)OCH₂C≡CCH₂CH₃ | Z-6233 | —C(═O)—Ph | —C(═O)OCH₂C≡CCH₂CH₃ | Z-6234 | —C(═O)—4-OCF₃—Ph | —C(═O)OCH₂C≡CCH₂CH₃ |
| Z-6235 | —C(═O)—c-Hex | —C(═O)OC(CH₃)₂C≡CH | Z-6236 | —C(═O)—Ph | —C(═O)OC(CH₃)₂C≡CH | Z-6237 | —C(═O)—4-OCF₃—Ph | —C(═O)OC(CH₃)₂C≡CH |
| Z-6238 | —C(═O)—c-Hex | —C(═O)OCH₂C≡CHCF₂H | Z-6239 | —C(═O)—Ph | —C(═O)OCH₂C≡CHCF₂H | Z-6240 | —C(═O)—4-OCF₃—Ph | —C(═O)OCH₂C≡CHCF₂H |
| Z-6241 | —C(═O)—c-Hex | —C(═O)OCH₂C≡CCF₃ | Z-6242 | —C(═O)—Ph | —C(═O)OCH₂C≡CCF₃ | Z-6243 | —C(═O)—4-OCF₃—Ph | —C(═O)OCH₂C≡CCF₃ |
| Z-6244 | —C(═O)—c-Hex | —C(═O)Ph | Z-6245 | —C(═O)—Ph | —C(═O)Ph | Z-6246 | —C(═O)—4-OCF₃—Ph | —C(═O)Ph |
| Z-6247 | —C(═O)—c-Hex | —C(═O)(2-Py) | Z-6248 | —C(═O)—Ph | —C(═O)(2-Py) | Z-6249 | —C(═O)—4-OCF₃—Ph | —C(═O)(2-Py) |
| Z-6250 | —C(═O)—c-Hex | —C(═O)(3-Py) | Z-6251 | —C(═O)—Ph | —C(═O)(3-Py) | Z-6252 | —C(═O)—4-OCF₃—Ph | —C(═O)(3-Py) |
| Z-6253 | —C(═O)—c-Hex | —C(═O)(4-Py) | Z-6254 | —C(═O)—Ph | —C(═O)(4-Py) | Z-6255 | —C(═O)—4-OCF₃—Ph | —C(═O)(4-Py) |
| Z-6256 | —C(═O)—c-Hex | —C(═O)CF₂Me | Z-6257 | —C(═O)—Ph | —C(═O)CF₂Me | Z-6258 | —C(═O)—4-OCF₃—Ph | —C(═O)CF₂Me |
| Z-6259 | —C(═O)—c-Hex | —C(═O)NMe2 | Z-6260 | —C(═O)—Ph | —C(═O)NMe2 | Z-6261 | —C(═O)—4-OCF₃—Ph | —C(═O)NMe2 |
| Z-6262 | —C(═O)—c-Hex | —C(═O)—(1-CF₃—c-Pr) | Z-6263 | —C(═O)—Ph | —C(═O)—(1-CF₃—c-Pr) | Z-6264 | —C(═O)—4-OCF₃—Ph | —C(═O)—(1-CF₃—c-Pr) |
| Z-6265 | —C(═O)—c-Hex | —C(═O)—(1-F—c-Pr) | Z-6266 | —C(═O)—Ph | —C(═O)—(1-F—c-Pr) | Z-6267 | —C(═O)—4-OCF₃—Ph | —C(═O)—(1-F—c-Pr) |
| Z-6268 | —C(═O)—c-Hex | —SO₂Me | Z-6269 | —C(═O)—Ph | —SO₂Me | Z-6270 | —C(═O)—4-OCF₃—Ph | —SO₂Me |
| Z-6271 | —C(═O)—c-Hex | —C(═O)CF═CH₂ | Z-6272 | —C(═O)—Ph | —C(═O)CF═CH₂ | Z-6273 | —C(═O)—4-OCF₃—Ph | —C(═O)CF═CH₂ |
| Z-6274 | —C(═O)—c-Hex | —C(═O)—(4-Cl—Ph) | Z-6275 | —C(═O)—Ph | —C(═O)—(4-Cl—Ph) | Z-6276 | —C(═O)—4-OCF₃—Ph | —C(═O)—(4-Cl—Ph) |
| Z-6277 | —C(═O)—c-Hex | —C(═O)—(3-Cl—Ph) | Z-6278 | —C(═O)—Ph | —C(═O)—(3-Cl—Ph) | Z-6279 | —C(═O)—4-OCF₃—Ph | —C(═O)—(3-Cl—Ph) |
| Z-6280 | —C(═O)—c-Hex | —C(═O)—(3-CF₃—Ph) | Z-6281 | —C(═O)—Ph | —C(═O)—(3-CF₃—Ph) | Z-6282 | —C(═O)—4-OCF₃—Ph | —C(═O)—(3-CF₃—Ph) |
| Z-6283 | —C(═O)—c-Hex | —C(═O)—(2-Cl—Ph) | Z-6284 | —C(═O)—Ph | —C(═O)—(2-Cl—Ph) | Z-6285 | —C(═O)—4-OCF₃—Ph | —C(═O)—(2-Cl—Ph) |
| Z-6286 | —C(═O)—c-Hex | —C(═O)—(2-CF₃—Ph) | Z-6287 | —C(═O)—Ph | —C(═O)—(2-CF₃—Ph) | Z-6288 | —C(═O)—4-OCF₃—Ph | —C(═O)—(2-CF₃—Ph) |
| Z-6289 | —C(═O)—c-Hex | —C(═O)—(4-CF₃—Ph) | Z-6290 | —C(═O)—Ph | —C(═O)—(4-CF₃—Ph) | Z-6291 | —C(═O)—4-OCF₃—Ph | —C(═O)—(4-CF₃—Ph) |
| Z-6292 | —C(═O)—c-Hex | —C(═O)—(3-F—Ph) | Z-6293 | —C(═O)—Ph | —C(═O)—(3-F—Ph) | Z-6294 | —C(═O)—4-OCF₃—Ph | —C(═O)—(3-F—Ph) |
| Z-6295 | —C(═O)—c-Hex | —C(═O)—(4-F—Ph) | Z-6296 | —C(═O)—Ph | —C(═O)—(4-F—Ph) | Z-6297 | —C(═O)—4-OCF₃—Ph | —C(═O)—(4-F—Ph) |
| Z-6298 | —C(═O)—c-Hex | —C(═O)—(2-F—Ph) | Z-6299 | —C(═O)—Ph | —C(═O)—(2-F—Ph) | Z-6300 | —C(═O)—4-OCF₃—Ph | —C(═O)—(2-F—Ph) |
| Z-6301 | —C(═O)—c-Hex | —C(═O)—(4-OCF₃—Ph) | Z-6302 | —C(═O)—Ph | —C(═O)—(4-OCF₃—Ph) | Z-6303 | —C(═O)—4-OCF₃—Ph | —C(═O)—(4-OCF₃—Ph) |
| Z-6304 | —C(═O)—c-Hex | —C(═O)—(6-Cl—3-Py) | Z-6305 | —C(═O)—Ph | —C(═O)—(6-Cl—3-Py) | Z-6306 | —C(═O)—4-OCF₃—Ph | —C(═O)—(6-Cl—3-Py) |
| Z-6307 | —C(═O)—c-Hex | —C(═O)—(6-CF3—2-Py) | Z-6308 | —C(═O)—Ph | —C(═O)—(6-CF3—2-Py) | Z-6309 | —C(═O)—4-OCF₃—Ph | —C(═O)—(6-CF3—2-Py) |
| Z-6310 | —C(═O)—c-Hex | —C(═O)—(1-CN—c-Pr) | Z-6311 | —C(═O)—Ph | —C(═O)—(1-CN—c-Pr) | Z-6312 | —C(═O)—4-OCF₃—Ph | —C(═O)—(1-CN—c-Pr) |
| Z-6313 | —C(═O)—c-Hex | —C(═O)—(3-Cl—2-Py) | Z-6314 | —C(═O)—Ph | —C(═O)—(3-Cl—2-Py) | Z-6315 | —C(═O)—4-OCF₃—Ph | —C(═O)—(3-Cl—2-Py) |
| Z-6316 | —C(═O)—c-Hex | —C(═O)—(2-Me—Ph) | Z-6317 | —C(═O)—Ph | —C(═O)—(2-Me—Ph) | Z-6318 | —C(═O)—4-OCF₃—Ph | —C(═O)—(2-Me—Ph) |
| Z-6319 | —C(═O)—c-Hex | —C(═O)—(3-Me—Ph) | Z-6320 | —C(═O)—Ph | —C(═O)—(3-Me—Ph) | Z-6321 | —C(═O)—4-OCF₃—Ph | —C(═O)—(3-Me—Ph) |
| Z-6322 | —C(═O)—c-Hex | —C(═O)—(4-Me—Ph) | Z-6323 | —C(═O)—Ph | —C(═O)—(4-Me—Ph) | Z-6324 | —C(═O)—4-OCF₃—Ph | —C(═O)—(4-Me—Ph) |
| Z-6325 | —C(═O)—c-Hex | —C(═O)—(2-MeO—Ph) | Z-6326 | —C(═O)—Ph | —C(═O)—(2-MeO—Ph) | Z-6327 | —C(═O)—4-OCF₃—Ph | —C(═O)—(2-MeO—Ph) |
| Z-6328 | —C(═O)—c-Hex | —C(═O)—(3-MeO—Ph) | Z-6329 | —C(═O)—Ph | —C(═O)—(3-MeO—Ph) | Z-6330 | —C(═O)—4-OCF₃—Ph | —C(═O)—(3-MeO—Ph) |
| Z-6331 | —C(═O)—c-Hex | —C(═O)—(4-MeO—Ph) | Z-6332 | —C(═O)—Ph | —C(═O)—(4-MeO—Ph) | Z-6333 | —C(═O)—4-OCF₃—Ph | —C(═O)—(4-MeO—Ph) |
| Z-6334 | —C(═O)—c-Hex | —C(═O)—(4-Cl—2-Py) | Z-6335 | —C(═O)—Ph | —C(═O)—(4-Cl—2-Py) | Z-6336 | —C(═O)—4-OCF₃—Ph | —C(═O)—(4-Cl—2-Py) |
| Z-6337 | —C(═O)—c-Hex | —C(═O)—(5-Cl—2-Py) | Z-6338 | —C(═O)—Ph | —C(═O)—(5-Cl—2-Py) | Z-6339 | —C(═O)—4-OCF₃—Ph | —C(═O)—(5-Cl—2-Py) |
| Z-6340 | —C(═O)—c-Hex | —C(═O)—(6-Cl—2-Py) | Z-6341 | —C(═O)—Ph | —C(═O)—(6-Cl—2-Py) | Z-6342 | —C(═O)—4-OCF₃—Ph | —C(═O)—(6-Cl—2-Py) |
| Z-6343 | —C(═O)—c-Hex | —C(═O)—(2-Cl—3-Py) | Z-6344 | —C(═O)—Ph | —C(═O)—(2-Cl—3-Py) | Z-6345 | —C(═O)—4-OCF₃—Ph | —C(═O)—(2-Cl—3-Py) |
| Z-6346 | —C(═O)—c-Hex | —C(═O)—(2-Cl—4-Py) | Z-6347 | —C(═O)—Ph | —C(═O)—(2-Cl—4-Py) | Z-6348 | —C(═O)—4-OCF₃—Ph | —C(═O)—(2-Cl—4-Py) |
| Z-6349 | —C(═O)—c-Hex | —C(═O)—(3-Cl—4-Py) | Z-6350 | —C(═O)—Ph | —C(═O)—(3-Cl—4-Py) | Z-6351 | —C(═O)—4-OCF₃—Ph | —C(═O)—(3-Cl—4-Py) |
| Z-6352 | —C(═O)—c-Hex | —C(═O)—(3,4-di-Me—Ph) | Z-6353 | —C(═O)—Ph | —C(═O)—(3,4-di-Me—Ph) | Z-6354 | —C(═O)—4-OCF₃—Ph | —C(═O)—(3,4-di-Me—Ph) |
| Z-6355 | —C(═O)—c-Hex | —C(═O)—(3,5-di-Me—Ph) | Z-6356 | —C(═O)—Ph | —C(═O)—(3,5-di-Me—Ph) | Z-6357 | —C(═O)—4-OCF₃—Ph | —C(═O)—(3,5-di-Me—Ph) |
| Z-6358 | —C(═O)—c-Hex | —C(═O)—(4-Cl—3-Py) | Z-6359 | —C(═O)—Ph | —C(═O)—(4-Cl—3-Py) | Z-6360 | —C(═O)—4-OCF₃—Ph | —C(═O)—(4-Cl—3-Py) |
| Z-6361 | —C(═O)—c-Hex | —C(═O)—(5-Cl—3-Py) | Z-6362 | —C(═O)—Ph | —C(═O)—(5-Cl—3-Py) | Z-6363 | —C(═O)—4-OCF₃—Ph | —C(═O)—(5-Cl—3-Py) |
| Z-6364 | —C(═O)—c-Hex | —C(═O)—(4-Pyrimidine) | Z-6365 | —C(═O)—Ph | —C(═O)—(4-Pyrimidine) | Z-6366 | —C(═O)—4-OCF₃—Ph | —C(═O)—(4-Pyrimidine) |
| Z-6367 | —C(═O)—c-Hex | —C(═O)—(2-Cl—4-Pyrimidine) | Z-6368 | —C(═O)—Ph | —C(═O)—(2-Cl—4-Pyrimidine) | Z-6369 | —C(═O)—4-OCF₃—Ph | —C(═O)—(2-Cl—4-Pyrimidine) |
| Z-6370 | —C(═O)—c-Hex | —C(═O)—(4-Et—Ph) | Z-6371 | —C(═O)—Ph | —C(═O)—(4-Et—Ph) | Z-6372 | —C(═O)—4-OCF₃—Ph | —C(═O)—(4-Et—Ph) |
| Z-6373 | —C(═O)—c-Hex | —C(═O)—(2-Me—4-Pyrimidine) | Z-6374 | —C(═O)—Ph | —C(═O)—(2-Me—4-Pyrimidine) | Z-6375 | —C(═O)—4-OCF₃—Ph | —C(═O)—(2-Me—4-Pyrimidine) |
| Z-6376 | —C(═O)—c-Hex | —C(═O)—(6-Me—4-Pyrimidine) | Z-6377 | —C(═O)—Ph | —C(═O)—(6-Me—4-Pyrimidine) | Z-6378 | —C(═O)—4-OCF₃—Ph | —C(═O)—(6-Me—4-Pyrimidine) |
| Z-6379 | —C(═O)—c-Hex | —C(═O)—(6-Me—2-Py) | Z-6380 | —C(═O)—Ph | —C(═O)—(6-Me—2-Py) | Z-6381 | —C(═O)—4-OCF₃—Ph | —C(═O)—(6-Me—2-Py) |
| Z-6382 | —C(═O)—c-Hex | —C(═O)—(2-CF₃—4-Pyrimidine) | Z-6383 | —C(═O)—Ph | —C(═O)—(2-CF₃—4-Pyrimidine) | Z-6384 | —C(═O)—4-OCF₃—Ph | —C(═O)—(2-CF₃—4-Pyrimidine) |
| Z-6385 | —C(═O)—c-Hex | —C(═O)—(3-Pyridazine) | Z-6386 | —C(═O)—Ph | —C(═O)—(3-Pyridazine) | Z-6387 | —C(═O)—4-OCF₃—Ph | —C(═O)—(3-Pyridazine) |
| Z-6388 | —C(═O)—c-Hex | —C(═O)—(1-Me—c-Pr) | Z-6389 | —C(═O)—Ph | —C(═O)—(1-Me—c-Pr) | Z-6390 | —C(═O)—4-OCF₃—Ph | —C(═O)—(1-Me—c-Pr) |
| Z-6391 | —C(═O)—c-Hex | —C(═O)—(1-CF₃—c-Bu) | Z-6392 | —C(═O)—Ph | —C(═O)—(1-CF₃—c-Bu) | Z-6393 | —C(═O)—4-OCF₃—Ph | —C(═O)—(1-CF₃—c-Bu) |
| Z-6394 | —C(═O)—c-Hex | —C(═O)—(2-Pyrimidine) | Z-6395 | —C(═O)—Ph | —C(═O)—(2-Pyrimidine) | Z-6396 | —C(═O)—4-OCF₃—Ph | —C(═O)—(2-Pyrimidine) |
| Z-6397 | —C(═O)—c-Hex | —C(═O)—(2-Pyrazine) | Z-6398 | —C(═O)—Ph | —C(═O)—(2-Pyrazine) | Z-6399 | —C(═O)—4-OCF₃—Ph | —C(═O)—(2-Pyrazine) |
| Z-6400 | —C(═O)—c-Hex | —C(═O)CH═CHOEt | Z-6401 | —C(═O)—Ph | —C(═O)CH═CHOEt | Z-6402 | —C(═O)—4-OCF₃—Ph | —C(═O)CH═CHOEt |
| Z-6403 | —C(═O)—c-Hex | —C(═O)CH₂CHCF₃CF₃ | Z-6404 | —C(═O)—Ph | —C(═O)CH₂CHCF₃CF₃ | Z-6405 | —C(═O)—4-OCF₃—Ph | —C(═O)CH₂CHCF₃CF₃ |
| Z-6406 | —C(═O)—c-Hex | —C(═O)CH₂—(c-Pr) | Z-6407 | —C(═O)—Ph | —C(═O)CH₂—(c-Pr) | Z-6408 | —C(═O)—4-OCF₃—Ph | —C(═O)CH₂—(c-Pr) |
| Z-6409 | —C(═O)—c-Hex | —C(═O)—(2,2,3,3-tetra-Me—c-Pr) | Z-6410 | —C(═O)—Ph | —C(═O)—(2,2,3,3-tetra-Me—c-Pr) | Z-6411 | —C(═O)—4-OCF₃—Ph | —C(═O)—(2,2,3,3-tetra-Me—c-Pr) |
| Z-6412 | —C(═O)—c-Hex | —C(═O)CMe₂CF₃ | Z-6413 | —C(═O)—Ph | —C(═O)CMe₂CF₃ | Z-6414 | —C(═O)—4-OCF₃—Ph | —C(═O)CMe₂CF₃ |

Hereinafter, methods for producing compounds represented by Formula (1-1) and Formula (1-2) are exemplified in a production method A to a production method AP. The method for producing a compound of the present invention is not limited thereto.

### Production Method A

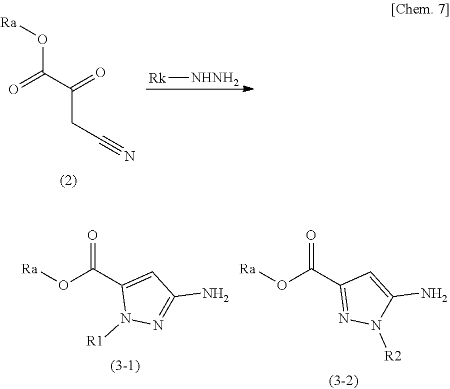

In the formula, Rk is R1 or R2, R1 and R2 represents a hydrogen atom, a C1 to C6 alkyl group optionally substituted with a substituent A, a C1 to C6 haloalkyl group, a C3 to C8 cycloalkyl group optionally substituted with a substituent B, a C2 to C6 alkenyl group optionally substituted with a substituent A, a C2 to C6 haloalkenyl group, a C2 to C6 alkynyl group optionally substituted with a substituent A, a C2 to C6 haloalkynyl group, a phenyl group optionally substituted with 0 to 5 substituents B, a 5- or 6-membered aromatic heterocyclic group optionally substituted with 0 to 4 substituents B, Rx1Rx2NC(═O)— (where Rx1 and Rx2 each independently represent a hydrogen atom, a hydroxyl group, a cyano group, a C1 to C6 alkyl group optionally substituted with a substituent A, a C1 to C6 haloalkyl group, a C3 to C8 cycloalkyl group optionally substituted with a substituent B, a C2 to C6 alkenyl group optionally substituted with a substituent A, a C2 to C6 haloalkenyl group, a C2 to C6 alkynyl group optionally substituted with a substituent A, a C2 to C6 haloalkynyl group, a C1 to C6 alkoxy group, a C1 to C6 haloalkoxy group, a C3 to C8 cycloalkoxy group, a phenyl group optionally substituted with 0 to 5 substituents B, or a 5- or 6-membered aromatic heterocyclic group optionally substituted with 0 to 4 substituents B, or Rx1 and Rx2 together with a nitrogen atom to which they are bonded form an aziridinyl group, an azetidinyl group, a pyrrolidinyl group, a piperidinyl group, a piperazinyl group, a morpholinyl group, a thiomorpholinyl group, a dioxothiomorpholinyl group, a thiazolidinyl group, an azepanyl group, or an azocanyl group), Rx3C(═O)— (where Rx3 represents a hydrogen atom, a hydroxyl group, a halogen atom, a C1 to C6 alkyl group optionally substituted with a substituent A, a C1 to C6 haloalkyl group, a C3 to C8 cycloalkyl group optionally substituted with a substituent B, a C2 to C6 alkenyl group optionally substituted with a substituent A, a C2 to C6 haloalkenyl group, a C2 to C6 alkynyl group optionally substituted with a substituent A, a C2 to C6 haloalkynyl group, a phenyl group optionally substituted with 0 to 5 substituents B, or a 5- or 6-membered aromatic heterocyclic group optionally substituted with 0 to 4 substituents B), Rx3OC(═O)— (where Rx3 has the same meaning as above), Rx4S(O)p- (where Rx4 represents a hydroxyl group, a halogen atom, a C1 to C6 alkyl group optionally substituted with a substituent A, a C1 to C6 haloalkyl group, a C3 to C8 cycloalkyl group optionally substituted with a substituent B, a C2 to C6 alkenyl group optionally substituted with a substituent A, a C2 to C6 haloalkenyl group, a C2 to C6 alkynyl group optionally substituted with a substituent A, a C2 to C6 haloalkynyl group, or a phenyl group optionally substituted with 0 to 5 substituents B, and p represents an integer of 0, 1, or 2), or a 3- to 6-membered ring group containing one or two oxygen atoms, and Ra represents a hydrogen atom or a C1 to C6 alkyl group optionally substituted with a substituent A.

A production method A is a method for obtaining compounds represented by Formula (3-1) and Formula (3-2) including a production intermediate of the compound of the present invention, the production method comprising reacting a compound represented by Formula (2) with Rk-NHNH₂ in a solvent.

Regarding the compound represented by Formula (3-1) and the compound represented by Formula (3-2), a ratio is not particularly limited, and either one may be used alone or a mixture may be used in any ratio.

Rk-NHNH₂ used in the present reaction can be obtained as a commercially available product or produced by a known method. Rk-NHNH₂ may form a salt with an acidic compound such as hydrochloric acid or hydrobromic acid, and is not particularly limited as long as the intended reaction proceeds.

The amount of Rk-NHNH₂ used in the present reaction may be 1 equivalent or more with respect to the compound represented by Formula (2), is not particularly limited as long as the intended reaction proceeds, and is preferably 1 equivalent or more and 20 equivalents or less.

The present reaction can be performed in the presence of an acid. Examples of the acid to be used include inorganic acids such as hydrochloric acid and sulfuric acid, and organic acids such as acetic acid, trifluoroacetic acid, methanesulfonic acid, and p-toluenesulfonic acid, and are not particularly limited as long as the intended reaction proceeds, but acetic acid or trifluoroacetic acid is preferable. In addition, when a salt of Rk-NHNH₂ and an acidic compound is used, the use of an acid is not essential.

The amount of acid used in the present reaction may be 1 equivalent or more with respect to the compound represented by Formula (2), is not particularly limited as long as the intended reaction proceeds, and is preferably 1 equivalent or more and 50 equivalents or less. In addition, when the acid to be used is a liquid, it can also be used as a solvent.

A solvent can be used in the present reaction, but is not necessarily required.

The solvent used in the present reaction is not particularly limited as long as the intended reaction proceeds, and examples thereof include acidic solvents such as acetic acid and methanesulfonic acid, ether-based solvents such as diethyl ether, diisopropyl ether, methyl-t-butyl ether, dimethoxyethane, tetrahydrofuran, and dioxane, alcohol-based solvents such as methanol, ethanol, isopropanol, and trifluoroethanol, benzene-based solvents such as benzene, toluene, xylene, mesitylene, chlorobenzene, and dichlorobenzene, ester-based solvents such as ethyl acetate, isopropyl acetate, and butyl acetate, nitrile-based solvents such as acetonitrile, amide-based solvents such as N-methylpyrrolidone, N,N-dimethylformamide, and N,N-dimethylacetamide, urea-based solvents such as 1,3-dimethyl-2 imidazolidinone, and halogen-based solvents such as dichloromethane, dichloroethane, chloroform, and carbon tetrachloride. These solvents can be used alone or as a mixture of two or more kinds thereof in an arbitrary ratio. The solvent is preferably an acidic solvent or an alcohol-based solvent, and more preferably ethanol or trifluoroethanol.

The amount of solvent used in the present reaction is not particularly limited as long as the desired reaction proceeds, and is usually 1 to 200 times by weight with respect to the compound represented by Formula (2)

The temperature for performing the present reaction is not particularly limited as long as the intended reaction proceeds, but is usually 0° C. or higher and 180° C. or lower or below a boiling point of the solvent.

As the post-treatment of the reaction, a liquid separation operation can be performed by adding water or an appropriate aqueous solution to the reaction mixture. When an aqueous solution is used, an acidic aqueous solution in which hydrochloric acid, sulfuric acid, ammonium chloride, or the like is dissolved, an alkaline aqueous solution in which potassium hydroxide, sodium hydroxide, sodium carbonate, potassium carbonate, sodium hydrogen carbonate, potassium hydrogen carbonate, or the like is dissolved, a saline solution, or the like can be arbitrarily used. In the liquid separation operation, it is possible to add a solvent that is not compatible with water, such as a benzene-based solvent such as toluene, xylene, benzene, chlorobenzene, or dichlorobenzene, an ester-based solvent such as ethyl acetate, isopropyl acetate, or butyl acetate, an ether-based solvent such as diethyl ether, diisopropyl ether, or methyl-t-butyl ether, a halogen-based solvent such as dichloromethane, dichloroethane, chloroform, or carbon tetrachloride, or a hydrocarbon-based solvent such as hexane, heptane, cyclohexane, or methylcyclohexane, as necessary. In addition, these solvents can be used alone, or two or more kinds thereof can be mixed at an arbitrary ratio. The number of times of liquid separation is not particularly limited, and the liquid separation can be performed according to the intended purity and yield.

In the reaction mixture containing the compounds represented by Formula (3-1) and Formula (3-2) obtained above, moisture can be removed with a desiccant such as sodium sulfate or magnesium sulfate, but it is not essential.

The reaction mixture containing the compounds represented by Formula (3-1) and Formula (3-2) obtained above can be subjected to solvent distillation under reduced pressure as long as the compounds are not decomposed.

A reaction mixture containing the compounds represented by Formula (3-1) and Formula (3-2) obtained after solvent distillation can be purified by washing, reprecipitation, recrystallization, column chromatography, or the like with an appropriate solvent. After purification, the compound of Formula (3-1) and the compound of Formula (3-2) may be separated, and may be appropriately set according to the intended purity.

### Production Method B

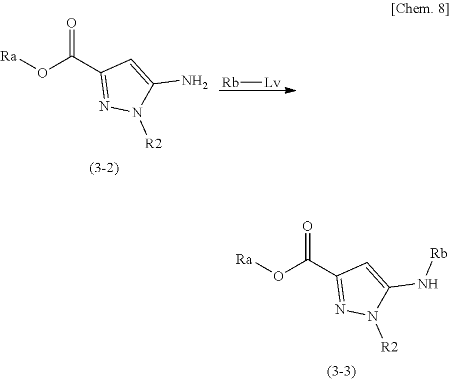

In the formula, Rb represents a C1 to C6 alkyl group optionally substituted with a substituent A, a C1 to C6 haloalkyl group, a C3 to C8 cycloalkyl group optionally substituted with a substituent B, a C2 to C6 alkenyl group optionally substituted with a substituent A, a C2 to C6 haloalkenyl group, a C2 to C6 alkynyl group optionally substituted with a substituent A, a C2 to C6 haloalkynyl group, Rx1Rx2NC(═O)— (where Rx1 and Rx2 have the same meanings as above), Rx3C(═O)— (where Rx3 has the same meaning as above), Rx3OC(═O)— (where Rx3 has the same meaning as above), or Rx4S(O)p- (where Rx4 and p have the same meanings as above), Lv represents a leaving group such as a methanesulfonyl group, a trifluoromethanesulfonyl group, a p-toluenesulfonyl group, a halogen atom, or an acid anhydride, and R2 and Ra have the same meanings as above.

A production method B is a method for obtaining a compound represented by Formula (3-3) including a production intermediate of the compound of the present invention, the production method comprising reacting a compound represented by Formula (3-2) with Rb-Lv in a solvent in the presence of a base.

Rb-Lv used in the present reaction can be obtained as a commercially available product or produced by a known method. When a compound in which Rb is Rx3C(═O)— (where Rx3 has the same meaning as above) or Rx4S(O)p- (where Rx4 and p have the same meanings as above) is used, Rb-Lv may use an acid anhydride.

The amount of Rb-Lv used in the present reaction may be 1 equivalent or more with respect to the compound represented by Formula (3-2), is not particularly limited as long as the intended reaction proceeds, and is preferably 1 equivalent or more and 10 equivalents or less.

Examples of the base used in the present reaction include inorganic bases such as sodium hydroxide, potassium hydroxide, sodium carbonate, potassium carbonate, cesium carbonate, and sodium hydride, organic bases such as triethylamine, tributylamine, diisopropylethylamine, 1,8-diazabicyclo[5.4.0]-7-undecene, 1,4-diazabicyclo[2.2.2]octane, and dimethylaminopyridine, metal hydrides such as sodium hydride, organic lithiums such as methyllithium, butyllithium, sec-butyllithium, t-butyllithium, and hexyllithium, and metal amides such as lithium diisopropylamide, hexamethyldisilazane lithium, hexamethyldisilazane sodium, and hexamethyldisilazane potassium.

The amount of base used in the present reaction may be 1 equivalent or more with respect to the compound represented by Formula (3-2), is not particularly limited as long as the intended reaction proceeds, and is preferably 1 equivalent or more and 10 equivalents or less.

The solvent used in the present reaction is not particularly limited as long as the intended reaction proceeds, and examples thereof include ether-based solvents such as diethyl ether, diisopropyl ether, methyl-t-butyl ether, dimethoxyethane, tetrahydrofuran, and dioxane, benzene-based solvents such as benzene, toluene, xylene, mesitylene, chlorobenzene, and dichlorobenzene, ester-based solvents such as ethyl acetate, isopropyl acetate, and butyl acetate, nitrile-based solvents such as acetonitrile, amide-based solvents such as N-methylpyrrolidone, N,N-dimethylformamide, and N,N-dimethylacetamide, urea-based solvents such as 1,3-dimethyl-2 imidazolidinone, and halogen-based solvents such as dichloromethane, dichloroethane, chloroform, and carbon tetrachloride. These solvents can be used alone or as a mixture of two or more kinds thereof in an arbitrary ratio.

The amount of solvent used in the present reaction is not particularly limited as long as the desired reaction proceeds, and is usually 3 to 200 times by weight with respect to the compound represented by Formula (3-2)

The temperature for performing the present reaction is not particularly limited as long as the intended reaction proceeds, but is usually −10° C. or higher and 180° C. or lower or below a boiling point of the solvent.

As the post-treatment of the reaction, a liquid separation operation can be performed by adding water or an appropriate aqueous solution to the reaction mixture. When an aqueous solution is used, an acidic aqueous solution in which hydrochloric acid, sulfuric acid, ammonium chloride, or the like is dissolved, an alkaline aqueous solution in which potassium hydroxide, sodium hydroxide, sodium carbonate, potassium carbonate, sodium hydrogen carbonate, potassium hydrogen carbonate, or the like is dissolved, an aqueous solution containing salts containing sulfur atoms such as sodium thiosulfate and sodium sulfite, a saline solution, or the like can be arbitrarily used. In the liquid separation operation, it is possible to add a solvent that is not compatible with water, such as a benzene-based solvent such as toluene, xylene, benzene, chlorobenzene, or dichlorobenzene, an ester-based solvent such as ethyl acetate, isopropyl acetate, or butyl acetate, an ether-based solvent such as diethyl ether, diisopropyl ether, or methyl-t-butyl ether, a halogen-based solvent such as dichloromethane, dichloroethane, or chloroform, or a hydrocarbon-based solvent such as hexane, heptane, cyclohexane, or methylcyclohexane, as necessary. In addition, these solvents can be used alone, or two or more kinds thereof can be mixed at an arbitrary ratio. The number of times of liquid separation is not particularly limited, and the liquid separation can be performed according to the intended purity and yield.

In the reaction mixture containing the compound represented by Formula (3-3) obtained above, moisture can be removed with a desiccant such as sodium sulfate or magnesium sulfate, but it is not essential.

The reaction mixture containing the compound represented by Formula (3-3) obtained above can be subjected to solvent distillation under reduced pressure as long as the compounds are not decomposed.

A reaction mixture containing the compound represented by Formula (3-3) obtained after solvent distillation can be purified by washing, reprecipitation, recrystallization, column chromatography, or the like with an appropriate solvent. It may be appropriately set according to the intended purity.

### Production Method C

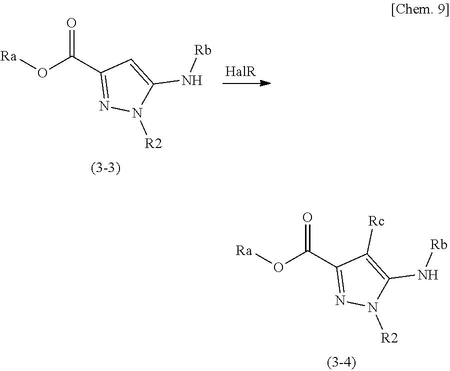

In the formula, Rc represents a halogen atom, HalR represents a halogenating agent, and R2, Ra, and Rb have the same meanings as above.

A production method C is a production method for obtaining a compound represented by Formula (3-4) in which Rc represents a halogen atom, the production method comprising reacting a compound represented by Formula (3-3) with a halogenation agent (HalR) in a solvent.

Examples of the halogenating agent used in the present reaction include selectfluor (N-fluoro-N′-triethylenediamine bis(tetrafluoroborate)), N-chlorosuccinimide, N-bromosuccinimide, N-iodosuccinimide, 1,3-dichloro-5,5-dimethylhydantoin, 1,3-dibromo-5,5-dimethylhydantoin, 1,3-diiodo-5,5-dimethylhydantoin, bromine, and iodine.

The amount of halogenating agent used in the present reaction may be 1 equivalent or more with respect to the compound represented by Formula (3-3), is not particularly limited as long as the intended reaction proceeds, and is preferably 1 equivalent or more and 10 equivalents or less. However, the amount of the halogenating agent containing hydantoin is not particularly limited as long as it is 0.5 equivalent or more and the desired reaction proceeds, and is preferably 1 equivalent or more and 5 equivalents or less.

When the halogenating agent used in the present reaction is an iodinating agent, an acid such as an inorganic acid such as hydrochloric acid or sulfuric acid, or an organic acid such as acetic acid, trifluoroacetic acid, methanesulfonic acid, or trifluoromethanesulfonic acid can be added.

When the amount of acid used when the halogenating agent used in the present reaction is an iodinating agent is 0.01 equivalents or more with respect to the compound represented by Formula (3-1), is not particularly limited as long as the intended reaction proceeds, and is preferably 0.1 equivalents or more and 3 equivalents or less.

The solvent used in the present reaction is not particularly limited as long as the intended reaction proceeds, and examples thereof include acidic solvents such as sulfuric acid, acetic acid, trifluoroacetic acid, methanesulfonic acid, and trifluoromethanesulfonic acid, ether-based solvents such as diethyl ether, diisopropyl ether, methyl-t-butyl ether, dimethoxyethane, tetrahydrofuran, and dioxane, alcohol-based solvents such as methanol, ethanol, and isopropanol, benzene-based solvents such as benzene, toluene, xylene, mesitylene, chlorobenzene, and dichlorobenzene, ester-based solvents such as ethyl acetate, isopropyl acetate, and butyl acetate, nitrile-based solvents such as acetonitrile, amide-based solvents such as N-methylpyrrolidone, N,N-dimethylformamide, and N,N-dimethylacetamide, urea-based solvents such as 1,3-dimethyl-2 imidazolidinone, and halogen-based solvents such as dichloromethane, dichloroethane, chloroform, and carbon tetrachloride. These solvents can be used alone or as a mixture of two or more kinds thereof in an arbitrary ratio.

The amount of solvent used in the present reaction is not particularly limited as long as the desired reaction proceeds, and is usually 1 to 200 times by weight with respect to the compound represented by Formula (3-3).

The temperature for performing the present reaction is not particularly limited as long as the intended reaction proceeds, but is usually 0° C. or higher and 150° C. or lower or below a boiling point of the solvent.

As the post-treatment of the reaction, a liquid separation operation can be performed by adding water or an appropriate aqueous solution to the reaction mixture. When an aqueous solution is used, an acidic aqueous solution in which hydrochloric acid, sulfuric acid, ammonium chloride, or the like is dissolved, an alkaline aqueous solution in which potassium hydroxide, sodium hydroxide, sodium carbonate, potassium carbonate, sodium hydrogen carbonate, potassium hydrogen carbonate, or the like is dissolved, an aqueous solution containing salts containing sulfur atoms such as sodium thiosulfate and sodium sulfite, a saline solution, or the like can be arbitrarily used. In the liquid separation operation, it is possible to add a solvent that is not compatible with water, such as a benzene-based solvent such as toluene, xylene, benzene, chlorobenzene, or dichlorobenzene, an ester-based solvent such as ethyl acetate, isopropyl acetate, or butyl acetate, an ether-based solvent such as diethyl ether, diisopropyl ether, or methyl-t-butyl ether, a halogen-based solvent such as dichloromethane, dichloroethane, chloroform, or carbon tetrachloride, or a hydrocarbon-based solvent such as hexane, heptane, cyclohexane, or methylcyclohexane, as necessary. In addition, these solvents can be used alone, or two or more kinds thereof can be mixed at an arbitrary ratio. The number of times of liquid separation is not particularly limited, and the liquid separation can be performed according to the intended purity and yield.

In the reaction mixture containing the compound represented by Formula (3-4) obtained above, moisture can be removed with a desiccant such as sodium sulfate or magnesium sulfate, but it is not essential.

The reaction mixture containing the compound represented by Formula (3-4) obtained above can be subjected to solvent distillation under reduced pressure as long as the compounds are not decomposed.

A reaction mixture containing the compound represented by Formula (3-4) obtained after solvent distillation can be purified by washing, reprecipitation, recrystallization, column chromatography, or the like with an appropriate solvent. It may be appropriately set according to the intended purity.

### Production Method D

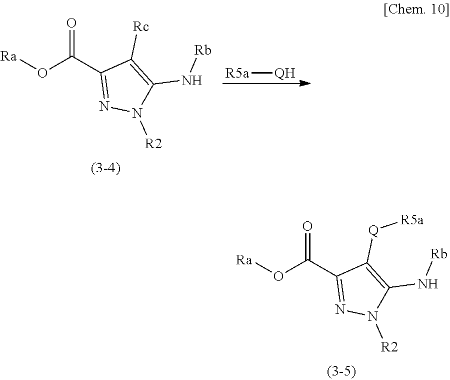

In the formula, R5a represents a C1 to C6 alkyl group optionally substituted with a substituent A, a C1 to C6 haloalkyl group, or a C3 to C8 cycloalkyl group optionally substituted with a substituent B, Q represents an oxygen atom or a sulfur atom, and R2, Ra, Rb, and Re have the same meanings as above.

A production method D is a method for synthesizing a compound represented by Formula (3-5), the production method comprising reacting a compound represented by Formula (3-4) with R5a-QH (where R5a has the same meaning as above) in a solvent in the presence of a base.

In the compound represented by Formula (3-4), Re is preferably a chlorine atom, a bromine atom, or an iodine atom, more preferably a bromine atom or an iodine atom, and particularly preferably an iodine atom.

R5a-QH (where R5a and Q have the same meanings as above) used in the present reaction can be obtained as a commercially available product or produced by a known method.

R5a-OH is preferably methanol, ethanol, propanol, isopropanol, 2,2,2-trifluoroethanol, or 2,2,3,3,3-pentafluoropropanol, and more preferably methanol, ethanol, or 2,2,2-trifluoroethanol.

R5a-SH is preferably methyl mercaptan, ethyl mercaptan, propyl mercaptan, isopropyl mercaptan, or butyl mercaptan, more preferably methyl mercaptan, ethyl mercaptan, or isopropyl mercaptan, and particularly preferably ethyl mercaptan.

The amount of R5a-QH used in the present reaction may be 1 equivalent or more with respect to the compound represented by Formula (3-4), is not particularly limited as long as the intended reaction proceeds, and is preferably 1 equivalent or more and 20 equivalents or less.

The present reaction can be performed by a coupling reaction using transition metals, and the transition metals used in the present reaction may have a ligand and are palladium such as palladium acetate, [1,1′-bis(diphenylphosphino)ferrocene]palladium dichloride, tris(dibenzylideneacetone)dipalladium, tetrakis(triphenylphosphine)palladium, or bis(triphenylphosphine)palladium dichloride.

The amount of transition metals used in the present reaction is usually 0.001 or more equivalents and 1 or less equivalent with respect to the compound represented by Formula (3-4), but is not particularly limited as long as the desired reaction proceeds.

In order to allow the present reaction to efficiently proceed, a phosphine ligand such as triphenylphosphine, 1,1′-bis(diphenylphosphino)ferrocene, 2-dicyclohexylphosphino-2′,4′,6′-triisopropylbiphenyl, 2-di-t-butylphosphino-2′,4′,6′-triisopropylbiphenyl, or 4,5-bis(diphenylphosphino)-9,9-dimethylxanthene can be added.

The amount of phosphine ligand used in the present reaction is usually 0.001 or more equivalents and 1 or less equivalent with respect to the compound represented by Formula (3-4), but is not particularly limited as long as the desired reaction proceeds.

Examples of the base used in the present reaction include inorganic bases such as sodium carbonate, potassium carbonate, and cesium carbonate, and organic bases such as triethylamine, tributylamine, and diisopropylethylamine.

The amount of base used in the present reaction may be 1 equivalent or more with respect to the compound represented by Formula (3-4), is not particularly limited as long as the intended reaction proceeds, and is preferably 1 equivalent or more and 50 equivalents or less.

The solvent used in the present reaction is not particularly limited as long as the intended reaction proceeds, and examples thereof include alcohol solvents such as methanol, ethanol, and t-butyl alcohol, ether-based solvents such as diethyl ether, diisopropyl ether, methyl-t-butyl ether, dimethoxyethane, tetrahydrofuran, and dioxane, and benzene-based solvents such as benzene, toluene, xylene, mesitylene, chlorobenzene, and dichlorobenzene. These solvents can be used alone or as a mixture of two or more kinds thereof in an arbitrary ratio.

The amount of solvent used in the present reaction is not particularly limited as long as the desired reaction proceeds, and is usually 3 to 200 times by weight with respect to the compound represented by Formula (3-4).

The temperature for performing the present reaction is not particularly limited as long as the intended reaction proceeds, but is usually 30° C. or higher and 180° C. or lower or below a boiling point of the solvent.

As the post-treatment of the reaction, a liquid separation operation can be performed by adding water or an appropriate aqueous solution to the reaction mixture. When an aqueous solution is used, an acidic aqueous solution in which hydrochloric acid, sulfuric acid, ammonium chloride, or the like is dissolved, an alkaline aqueous solution in which potassium hydroxide, sodium hydroxide, sodium carbonate, potassium carbonate, sodium hydrogen carbonate, potassium hydrogen carbonate, or the like is dissolved, a saline solution, or the like can be arbitrarily used. In the liquid separation operation, it is possible to add a solvent that is not compatible with water, such as a benzene-based solvent such as toluene, xylene, benzene, chlorobenzene, or dichlorobenzene, an ester-based solvent such as ethyl acetate, isopropyl acetate, or butyl acetate, an ether-based solvent such as diethyl ether, diisopropyl ether, or methyl-t-butyl ether, a halogen-based solvent such as dichloromethane, dichloroethane, chloroform, or carbon tetrachloride, or a hydrocarbon-based solvent such as hexane, heptane, cyclohexane, or methylcyclohexane, as necessary. In addition, these solvents can be used alone, or two or more kinds thereof can be mixed at an arbitrary ratio. The number of times of liquid separation is not particularly limited, and the liquid separation can be performed according to the intended purity and yield. In addition, it is also possible to remove insoluble matters by performing a filtration operation, but it is not essential.

In the reaction mixture containing the compound represented by Formula (3-5) obtained above, moisture can be removed with a desiccant such as sodium sulfate or magnesium sulfate, but it is not essential.

The reaction mixture containing the compound represented by Formula (3-5) obtained above can be subjected to solvent distillation under reduced pressure as long as the compounds are not decomposed.

A reaction mixture containing the compound represented by Formula (3-5) obtained after solvent distillation can be purified by washing, reprecipitation, recrystallization, column chromatography, or the like with an appropriate solvent. It may be appropriately set according to the intended purity.

### Production Method E

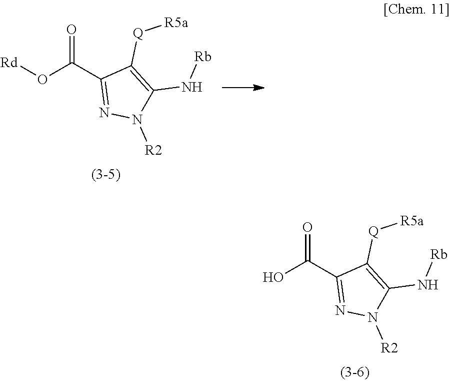

In the formula, Rd represents a C1 to C6 alkyl group optionally substituted with a substituent A, and R2, R5a, Q, and Rb have the same meanings as above.

A production method E is a method for synthesizing a compound represented by Formula (3-6), the production method comprising reacting a compound represented by Formula (3-5) in a solvent under acidic or basic conditions.

First, the reaction under acidic conditions will be described.

Examples of the acid used in the present reaction include inorganic acids such as hydrochloric acid, hydrobromic acid, and phosphoric acid, and organic acids such as acetic acid, methanesulfonic acid, p-toluenesulfonic acid, and trifluoroacetic acid. There is no particular limitation as long as the intended reaction proceeds.

The amount of acid used in the present reaction may be a catalytic amount, is not particularly limited as long as the intended reaction proceeds, and is preferably 0.01 equivalents or more with respect to the compound represented by Formula (3-5). In addition, a liquid acid can also be used as a solvent.

The solvent used in the present reaction is not particularly limited as long as the intended reaction proceeds, and examples thereof include an aqueous solvent, acidic solvents such as acetic acid and methanesulfonic acid, ether-based solvents such as diethyl ether, diisopropyl ether, methyl-t-butyl ether, dimethoxyethane, tetrahydrofuran, and dioxane, alcohol-based solvents such as methanol, ethanol, and isopropanol, benzene-based solvents such as benzene, toluene, xylene, mesitylene, chlorobenzene, and dichlorobenzene, ester-based solvents such as ethyl acetate, isopropyl acetate, and butyl acetate, nitrile-based solvents such as acetonitrile, amide-based solvents such as N-methylpyrrolidone, N,N-dimethylformamide, and N,N-dimethylacetamide, urea-based solvents such as 1,3-dimethyl-2 imidazolidinone, and halogen-based solvents such as dichloromethane, dichloroethane, chloroform, and carbon tetrachloride. These solvents can be used alone or as a mixture of two or more kinds thereof in an arbitrary ratio.

The amount of solvent used in the present reaction is not particularly limited as long as the desired reaction proceeds, and is usually 3 to 200 times by weight with respect to the compound represented by Formula (3-5).

The temperature for performing the present reaction is not particularly limited as long as the intended reaction proceeds, but is usually 0° C. or higher and 180° C. or lower or below a boiling point of the solvent.

Next, the reaction under basic conditions will be described.

Examples of the base used in the present reaction include inorganic bases such as lithium hydroxide, sodium hydroxide, and potassium hydroxide, and are not particularly limited as long as the intended reaction proceeds.

The base used in the present reaction may be 1 equivalent or more with respect to the compound represented by Formula (3-5), is not particularly limited as long as the intended reaction proceeds, and is preferably 1 equivalent or more and 30 equivalents or less.

The solvent used in the present reaction is not particularly limited as long as the intended reaction proceeds, and examples thereof include an aqueous solvent, ether-based solvents such as diethyl ether, diisopropyl ether, methyl-t-butyl ether, dimethoxyethane, tetrahydrofuran, and dioxane, alcohol-based solvents such as methanol, ethanol, and isopropanol, benzene-based solvents such as benzene, toluene, xylene, mesitylene, chlorobenzene, and dichlorobenzene, ester-based solvents such as ethyl acetate, isopropyl acetate, and butyl acetate, nitrile-based solvents such as acetonitrile, amide-based solvents such as N-methylpyrrolidone, N,N-dimethylformamide, and N,N-dimethylacetamide, urea-based solvents such as 1,3-dimethyl-2 imidazolidinone, and halogen-based solvents such as dichloromethane, dichloroethane, chloroform, and carbon tetrachloride. These solvents can be used alone or as a mixture of two or more kinds thereof in an arbitrary ratio.

The amount of solvent used in the present reaction is not particularly limited as long as the desired reaction proceeds, and is usually 3 to 200 times by weight with respect to the compound represented by Formula (3-5).

The temperature for performing the present reaction is not particularly limited as long as the intended reaction proceeds, but is usually −20° C. or higher and 180° C. or lower or below a boiling point of the solvent.

In the post-treatment of the reaction, the reaction under acidic conditions and the reaction under basic conditions can be performed by a common method. A liquid separation operation can be performed by adding water or an appropriate aqueous solution to the reaction mixture. When an aqueous solution is used, an acidic aqueous solution in which hydrochloric acid, sulfuric acid, or the like is dissolved, an alkaline aqueous solution in which potassium hydroxide, sodium hydroxide, sodium carbonate, potassium carbonate, sodium hydrogen carbonate, potassium hydrogen carbonate, or the like is dissolved, a saline solution, or the like can be arbitrarily used. In the liquid separation operation, it is possible to add a solvent that is not compatible with water, such as a benzene-based solvent such as toluene, xylene, benzene, chlorobenzene, or dichlorobenzene, an ester-based solvent such as ethyl acetate, isopropyl acetate, or butyl acetate, an ether-based solvent such as diethyl ether, diisopropyl ether, or methyl-t-butyl ether, a halogen-based solvent such as dichloromethane, dichloroethane, chloroform, or carbon tetrachloride, or a hydrocarbon-based solvent such as hexane, heptane, cyclohexane, or methylcyclohexane, as necessary. In addition, these solvents can be used alone, or two or more kinds thereof can be mixed at an arbitrary ratio. The number of times of liquid separation is not particularly limited, and the liquid separation can be performed according to the intended purity and yield.

In the reaction mixture containing the compound represented by Formula (3-6) obtained above, moisture can be removed with a desiccant such as sodium sulfate or magnesium sulfate, but it is not essential.

The reaction mixture containing the compound represented by Formula (3-6) obtained above can be subjected to solvent distillation under reduced pressure as long as the compounds are not decomposed.

A reaction mixture containing the compound represented by Formula (3-6) obtained after solvent distillation can be purified by washing, reprecipitation, recrystallization, column chromatography, or the like with an appropriate solvent. It may be appropriately set according to the intended purity.

### Production Method F

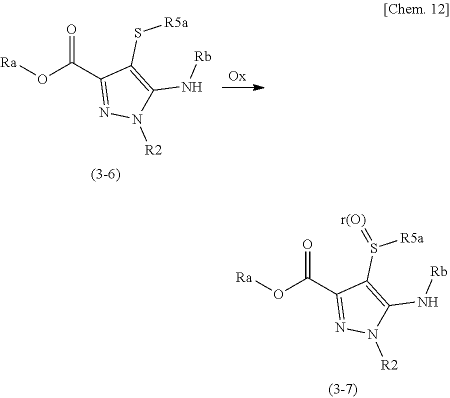

In the formula, r represents an integer of 1 or 2, Ox represents an oxidizing agent, and R2, R5a, Ra, and Rb have the same meanings as above.

A production method F is a method for obtaining a compound represented by Formula (3-7), the production method comprising reacting a compound represented by Formula (3-6) with an oxidizing agent (Ox) in a solvent.

Examples of the oxidizing agent used in the present reaction include hydrogen peroxide water and peroxides such as meta-chloroperbenzoic acid. In addition, transition metals such as sodium tungstate can also be added.

The amount of oxidizing agent used in the present reaction is usually 1.0 equivalent or more and less than 1.2 equivalents with respect to the compound represented by Formula (3-6) when producing a compound in which r is an integer of 1 in Formula (3-7), and is usually 2 equivalents or more and 10 equivalents or less when producing a compound in which r is an integer of 2 in Formula (3-7). In addition, when the transition metals are added, the amount of transition metals used is usually 0.001 equivalents or more and 1 equivalent or less.

The solvent used in the present reaction is not particularly limited as long as the intended reaction proceeds, and examples thereof include an aqueous solvent, acidic solvents such as acetic acid, benzene-based solvents such as benzene, toluene, xylene, mesitylene, chlorobenzene, and dichlorobenzene, nitrile-based solvents such as acetonitrile, and halogen-based solvents such as dichloromethane, dichloroethane, chloroform, and carbon tetrachloride. These solvents can be used alone or as a mixture of two or more kinds thereof in an arbitrary ratio.

The amount of solvent used in the present reaction is not particularly limited as long as the desired reaction proceeds, and is usually 3 to 200 times by weight with respect to the compound represented by Formula (3-6).

The temperature for performing the present reaction is not particularly limited as long as the intended reaction proceeds, but is usually −10° C. or higher and 120° C. or lower or below a boiling point of the solvent.

As the post-treatment of the reaction, a liquid separation operation can be performed by adding water or an appropriate aqueous solution to the reaction mixture. When an aqueous solution is used, an acidic aqueous solution in which hydrochloric acid, sulfuric acid, ammonium chloride, or the like is dissolved, an alkaline aqueous solution in which potassium hydroxide, sodium hydroxide, sodium carbonate, potassium carbonate, sodium hydrogen carbonate, potassium hydrogen carbonate, or the like is dissolved, an aqueous solution containing salts containing sulfur atoms such as sodium thiosulfate and sodium sulfite, a saline solution, or the like can be arbitrarily used. In the liquid separation operation, it is possible to add a solvent that is not compatible with water, such as a benzene-based solvent such as toluene, xylene, benzene, chlorobenzene, or dichlorobenzene, an ester-based solvent such as ethyl acetate, isopropyl acetate, or butyl acetate, an ether-based solvent such as diethyl ether, diisopropyl ether, or methyl-t-butyl ether, a halogen-based solvent such as dichloromethane, dichloroethane, chloroform, or carbon tetrachloride, or a hydrocarbon-based solvent such as hexane, heptane, cyclohexane, or methylcyclohexane, as necessary. In addition, these solvents can be used alone, or two or more kinds thereof can be mixed at an arbitrary ratio. The number of times of liquid separation is not particularly limited, and the liquid separation can be performed according to the intended purity and yield.

In the reaction mixture containing the compound represented by Formula (3-7) obtained above, moisture can be removed with a desiccant such as sodium sulfate or magnesium sulfate, but it is not essential.

The reaction mixture containing the compound represented by Formula (3-7) obtained above can be subjected to solvent distillation under reduced pressure as long as the compounds are not decomposed.

A reaction mixture containing the compound represented by Formula (3-7) obtained after solvent distillation can be purified by washing, reprecipitation, recrystallization, column chromatography, or the like with an appropriate solvent. It may be appropriately set according to the intended purity.

### Production Method G

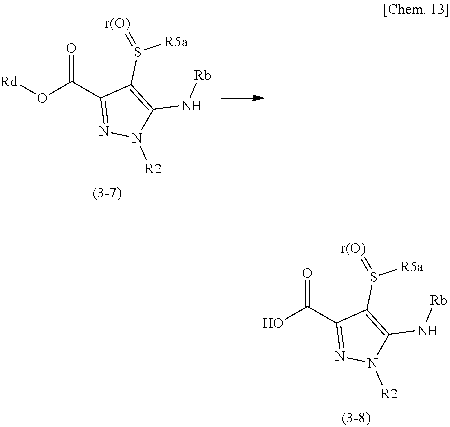

In the formula, Rd represents a C1 to C6 alkyl group optionally substituted with a substituent A, and R2, R5a, Rb, and r have the same meanings as above.

A production method G is a method for synthesizing a compound represented by Formula (3-8), the production method comprising reacting a compound represented by Formula (3-7) in a solvent under acidic or basic conditions.

First, the reaction under acidic conditions will be described.

Examples of the acid used in the present reaction include inorganic acids such as hydrochloric acid, hydrobromic acid, and phosphoric acid, and organic acids such as acetic acid, methanesulfonic acid, p-toluenesulfonic acid, and trifluoroacetic acid. There is no particular limitation as long as the intended reaction proceeds.

The amount of acid used in the present reaction may be a catalytic amount, is not particularly limited as long as the intended reaction proceeds, and is preferably 0.01 equivalents or more with respect to the compound represented by Formula (3-7). In addition, a liquid acid can also be used as a solvent.

The solvent used in the present reaction is not particularly limited as long as the intended reaction proceeds, and examples thereof include an aqueous solvent, acidic solvents such as acetic acid and methanesulfonic acid, ether-based solvents such as diethyl ether, diisopropyl ether, methyl-t-butyl ether, dimethoxyethane, tetrahydrofuran, and dioxane, alcohol-based solvents such as methanol, ethanol, and isopropanol, benzene-based solvents such as benzene, toluene, xylene, mesitylene, chlorobenzene, and dichlorobenzene, ester-based solvents such as ethyl acetate, isopropyl acetate, and butyl acetate, nitrile-based solvents such as acetonitrile, amide-based solvents such as N-methylpyrrolidone, N,N-dimethylformamide, and N,N-dimethylacetamide, urea-based solvents such as 1,3-dimethyl-2 imidazolidinone, and halogen-based solvents such as dichloromethane, dichloroethane, chloroform, and carbon tetrachloride. These solvents can be used alone or as a mixture of two or more kinds thereof in an arbitrary ratio.

The amount of solvent used in the present reaction is not particularly limited as long as the desired reaction proceeds, and is usually 3 to 200 times by weight with respect to the compound represented by Formula (3-7).

The temperature for performing the present reaction is not particularly limited as long as the intended reaction proceeds, but is usually 0° C. or higher and 180° C. or lower or below a boiling point of the solvent.

Next, the reaction under basic conditions will be described.

Examples of the base used in the present reaction include inorganic bases such as lithium hydroxide, sodium hydroxide, and potassium hydroxide, and are not particularly limited as long as the intended reaction proceeds.

The base used in the present reaction may be 1 equivalent or more with respect to the compound represented by Formula (3-7), is not particularly limited as long as the intended reaction proceeds, and is preferably 1 equivalent or more and 30 equivalents or less.

The solvent used in the present reaction is not particularly limited as long as the intended reaction proceeds, and examples thereof include an aqueous solvent, ether-based solvents such as diethyl ether, diisopropyl ether, methyl-t-butyl ether, dimethoxyethane, tetrahydrofuran, and dioxane, alcohol-based solvents such as methanol, ethanol, and isopropanol, benzene-based solvents such as benzene, toluene, xylene, mesitylene, chlorobenzene, and dichlorobenzene, ester-based solvents such as ethyl acetate, isopropyl acetate, and butyl acetate, nitrile-based solvents such as acetonitrile, amide-based solvents such as N-methylpyrrolidone, N,N-dimethylformamide, and N,N-dimethylacetamide, urea-based solvents such as 1,3-dimethyl-2 imidazolidinone, and halogen-based solvents such as dichloromethane, dichloroethane, chloroform, and carbon tetrachloride. These solvents can be used alone or as a mixture of two or more kinds thereof in an arbitrary ratio.

The amount of solvent used in the present reaction is not particularly limited as long as the desired reaction proceeds, and is usually 3 to 200 times by weight with respect to the compound represented by Formula (3-7).

The temperature for performing the present reaction is not particularly limited as long as the intended reaction proceeds, but is usually −20° C. or higher and 180° C. or lower or below a boiling point of the solvent.

In the post-treatment of the reaction, the reaction under acidic conditions and the reaction under basic conditions can be performed by a common method. A liquid separation operation can be performed by adding water or an appropriate aqueous solution to the reaction mixture. When an aqueous solution is used, an acidic aqueous solution in which hydrochloric acid, sulfuric acid, or the like is dissolved, an alkaline aqueous solution in which potassium hydroxide, sodium hydroxide, sodium carbonate, potassium carbonate, sodium hydrogen carbonate, potassium hydrogen carbonate, or the like is dissolved, a saline solution, or the like can be arbitrarily used. In the liquid separation operation, it is possible to add a solvent that is not compatible with water, such as a benzene-based solvent such as toluene, xylene, benzene, chlorobenzene, or dichlorobenzene, an ester-based solvent such as ethyl acetate, isopropyl acetate, or butyl acetate, an ether-based solvent such as diethyl ether, diisopropyl ether, or methyl-t-butyl ether, a halogen-based solvent such as dichloromethane, dichloroethane, chloroform, or carbon tetrachloride, or a hydrocarbon-based solvent such as hexane, heptane, cyclohexane, or methylcyclohexane, as necessary. In addition, these solvents can be used alone, or two or more kinds thereof can be mixed at an arbitrary ratio. The number of times of liquid separation is not particularly limited, and the liquid separation can be performed according to the intended purity and yield.

In the reaction mixture containing the compound represented by Formula (3-8) obtained above, moisture can be removed with a desiccant such as sodium sulfate or magnesium sulfate, but it is not essential.

The reaction mixture containing the compound represented by Formula (3-8) obtained above can be subjected to solvent distillation under reduced pressure as long as the compounds are not decomposed.

A reaction mixture containing the compound represented by Formula (3-8) obtained after solvent distillation can be purified by washing, reprecipitation, recrystallization, column chromatography, or the like with an appropriate solvent. It may be appropriately set according to the intended purity.

### Production Method H

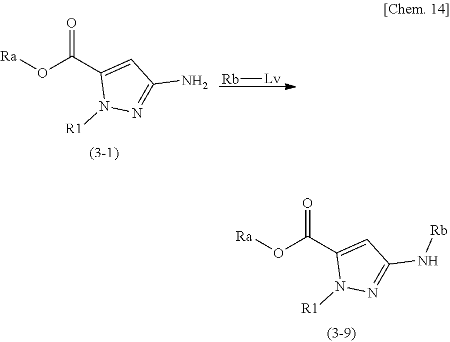

In the formula, Ra, R1, Rb, and Lv have the same meanings as above.

A production method H is a method for obtaining a compound represented by Formula (3-9) including a production intermediate of the compound of the present invention, the production method comprising reacting a compound represented by Formula (3-1) with Rb-Lv in a solvent in the presence of a base.

The production method H can be performed in accordance with the production method B by using the compound represented by Formula (3-2) instead of the compound represented by Formula (3-1) in the production method B.

### Production Method I

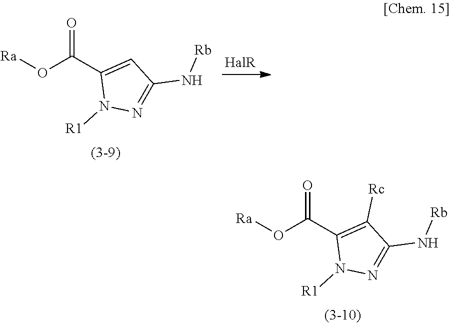

In the formula, Ra, R1, Rb, Re, and HalR have the same meanings as above.

A production method I is a production method for obtaining a compound represented by Formula (3-10) in which Rc represents a halogen atom, the production method comprising reacting a compound represented by Formula (3-9) with a halogenation agent (HalR) in a solvent.

The production method I can be performed in accordance with the production method C by using the compound represented by Formula (3-3) instead of the compound represented by Formula (3-9) in the production method C.

### Production Method J

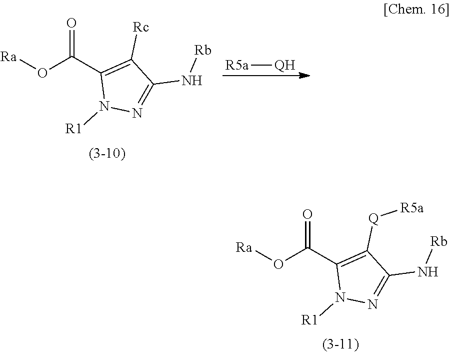

In the formula, R1, R5a, Q, Ra, Rb, and Rc have the same meanings as above.

A production method J is a method for synthesizing a compound represented by Formula (3-11), the production method comprising reacting a compound represented by Formula (3-10) with R5a-QH (where R5a and Q have the same meanings as above) in a solvent in the presence of a base.

The production method J can be performed in accordance with the production method D by using the compound represented by Formula (3-4) instead of the compound represented by Formula (3-10) in the production method D.

### Production Method K

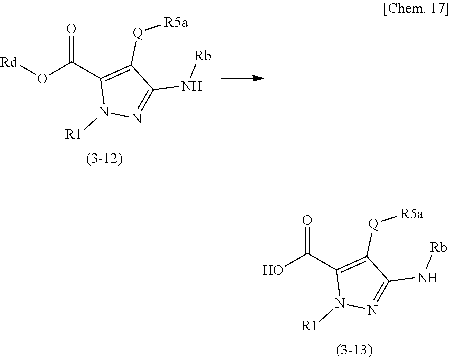

In the formula, R1, R5a, Rb, and Rd have the same meanings as above.

A production method K is a method for synthesizing a compound represented by Formula (3-13), the production method comprising reacting a compound represented by Formula (3-12) in a solvent under acidic or basic conditions.

The production method K can be performed in accordance with the production method E by using the compound represented by Formula (3-5) instead of the compound represented by Formula (3-12) in the production method E.

### Production Method L

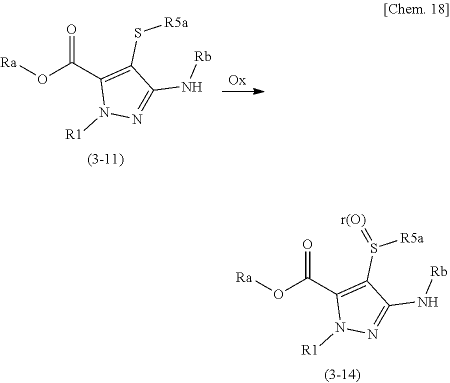

In the formula, R1, R5a, Ra, Rb, r, and Ox have the same meanings as above.

A production method L is a method for obtaining a compound represented by Formula (3-14), the production method comprising reacting a compound represented by Formula (3-11) and an oxidizing agent (Ox) in a solvent.

The production method L can be performed in accordance with the production method F by using the compound represented by Formula (3-6) instead of the compound represented by Formula (3-11) in the production method F.

### Production Method M

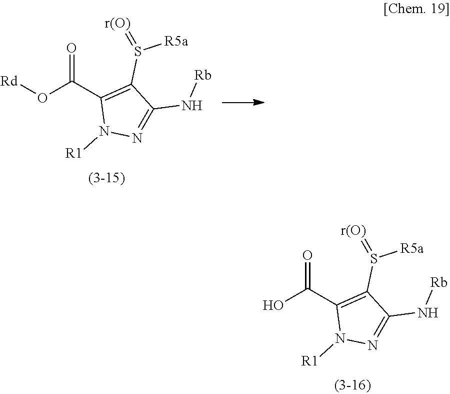

In the formula, R1, R5a, Rb, Rd, and r have the same meanings as above.

A production method M is a method for synthesizing a compound represented by Formula (3-16), the production method comprising reacting a compound represented by Formula (3-15) in a solvent under acidic or basic conditions.

The production method M can be performed in accordance with the production method G by using the compound represented by Formula (3-7) instead of the compound represented by Formula (3-15) in the production method G.

### Production Method N

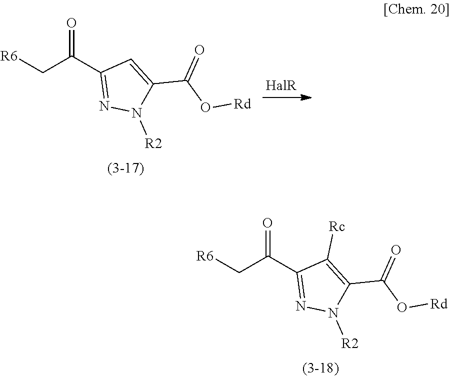

R6 in the formula represents a hydrogen atom, a cyano group, a halogen atom, a C1 to C6 alkyl group optionally substituted with a substituent A, a C1 to C6 haloalkyl group, a C3 to C8 cycloalkyl group optionally substituted with a substituent B, a C2 to C6 alkenyl group optionally substituted with a substituent A, a C2 to C6 haloalkenyl group, a C2 to C6 alkynyl group optionally substituted with a substituent A, or a C2 to C6 haloalkynyl group, and R2, Re, Rd, and HalR have the same meanings as above.

A production method N is a production method for obtaining a compound represented by Formula (3-18) in which Re represents a halogen atom, the production method comprising reacting a compound represented by Formula (3-17) with a halogenation agent (HalR) in a solvent.

The production method N can be performed in accordance with the production method C by using the compound represented by Formula (3-3) instead of the compound represented by Formula (3-17) in the production method C.

### Production Method 0

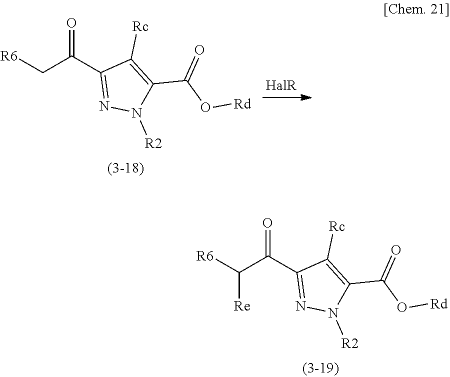

In the formula, Re represents a halogen atom, HalR represents a halogenating agent, and R2, R6, Rc, and Rd have the same meanings as above.

A production method O is a production method for obtaining a compound represented by Formula (3-19) in which Re represents a halogen atom, the production method comprising reacting a compound represented by Formula (3-18) with a halogenation agent (HalR) in a solvent.

Examples of the halogenating agent used in the present reaction include selectfluor (N-fluoro-N′-triethylenediamine bis(tetrafluoroborate)), N-chlorosuccinimide, N-bromosuccinimide, N-iodosuccinimide, 1,3-dichloro-5,5-dimethylhydantoin, 1,3-dibromo-5,5-dimethylhydantoin, 1,3-diiodo-5,5-dimethylhydantoin, bromine, iodine, trimethylphenylammonium tribromide, and benzyltrimethylammonium tribromide.

The amount of halogenating agent used in the present reaction may be 1 equivalent or more with respect to the compound represented by Formula (3-18), is not particularly limited as long as the intended reaction proceeds, and is preferably 1 equivalent or more and 10 equivalents or less. However, the amount of the halogenating agent containing hydantoin is not particularly limited as long as it is 0.5 equivalent or more and the desired reaction proceeds, and is preferably 1 equivalent or more and 5 equivalents or less.

When the halogenating agent used in the present reaction is an iodinating agent, an acid such as an inorganic acid such as hydrochloric acid or sulfuric acid, or an organic acid such as acetic acid, trifluoroacetic acid, methanesulfonic acid, or trifluoromethanesulfonic acid can be added.

When the amount of acid used when the halogenating agent used in the present reaction is an iodinating agent is 0.01 equivalents or more with respect to the compound represented by Formula (3-18), is not particularly limited as long as the intended reaction proceeds, and is preferably 0.1 equivalents or more and 3 equivalents or less.

The solvent used in the present reaction is not particularly limited as long as the intended reaction proceeds, and examples thereof include acidic solvents such as sulfuric acid, acetic acid, trifluoroacetic acid, methanesulfonic acid, and trifluoromethanesulfonic acid, ether-based solvents such as diethyl ether, diisopropyl ether, methyl-t-butyl ether, dimethoxyethane, tetrahydrofuran, and dioxane, alcohol-based solvents such as methanol, ethanol, and isopropanol, benzene-based solvents such as benzene, toluene, xylene, mesitylene, chlorobenzene, and dichlorobenzene, ester-based solvents such as ethyl acetate, isopropyl acetate, and butyl acetate, nitrile-based solvents such as acetonitrile, amide-based solvents such as N-methylpyrrolidone, N,N-dimethylformamide, and N,N-dimethylacetamide, urea-based solvents such as 1,3-dimethyl-2 imidazolidinone, and halogen-based solvents such as dichloromethane, dichloroethane, chloroform, and carbon tetrachloride. These solvents can be used alone or as a mixture of two or more kinds thereof in an arbitrary ratio.

The amount of solvent used in the present reaction is not particularly limited as long as the desired reaction proceeds, and is usually 1 to 200 times by weight with respect to the compound represented by Formula (3-18).

The temperature for performing the present reaction is not particularly limited as long as the intended reaction proceeds, but is usually 0° C. or higher and 150° C. or lower or below a boiling point of the solvent.

As the post-treatment of the reaction, a liquid separation operation can be performed by adding water or an appropriate aqueous solution to the reaction mixture. When an aqueous solution is used, an acidic aqueous solution in which hydrochloric acid, sulfuric acid, ammonium chloride, or the like is dissolved, an alkaline aqueous solution in which potassium hydroxide, sodium hydroxide, sodium carbonate, potassium carbonate, sodium hydrogen carbonate, potassium hydrogen carbonate, or the like is dissolved, an aqueous solution containing salts containing sulfur atoms such as sodium thiosulfate and sodium sulfite, a saline solution, or the like can be arbitrarily used. In the liquid separation operation, it is possible to add a solvent that is not compatible with water, such as a benzene-based solvent such as toluene, xylene, benzene, chlorobenzene, or dichlorobenzene, an ester-based solvent such as ethyl acetate, isopropyl acetate, or butyl acetate, an ether-based solvent such as diethyl ether, diisopropyl ether, or methyl-t-butyl ether, a halogen-based solvent such as dichloromethane, dichloroethane, chloroform, or carbon tetrachloride, or a hydrocarbon-based solvent such as hexane, heptane, cyclohexane, or methylcyclohexane, as necessary. In addition, these solvents can be used alone, or two or more kinds thereof can be mixed at an arbitrary ratio. The number of times of liquid separation is not particularly limited, and the liquid separation can be performed according to the intended purity and yield.

In the reaction mixture containing the compound represented by Formula (3-19) obtained above, moisture can be removed with a desiccant such as sodium sulfate or magnesium sulfate, but it is not essential.

The reaction mixture containing the compound represented by Formula (3-19) obtained above can be subjected to solvent distillation under reduced pressure as long as the compounds are not decomposed.

A reaction mixture containing the compound represented by Formula (3-19) obtained after solvent distillation can be purified by washing, reprecipitation, recrystallization, column chromatography, or the like with an appropriate solvent. It may be appropriately set according to the intended purity.

### Production Method P

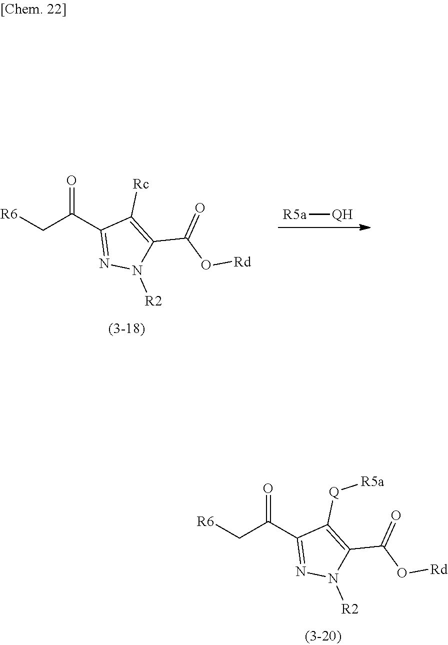

In the formula, R2, R5a, R6, Rc, and Rd have the same meanings as above.

A production method P is a method for synthesizing a compound represented by Formula (3-20), the production method comprising reacting a compound represented by Formula (3-18) with R5a-QH (where R5a and Q have the same meanings as above) in a solvent in the presence of a base.

The production method P can be performed in accordance with the production method D by using the compound represented by Formula (3-4) instead of the compound represented by Formula (3-18) in the production method D.

### Production Method Q

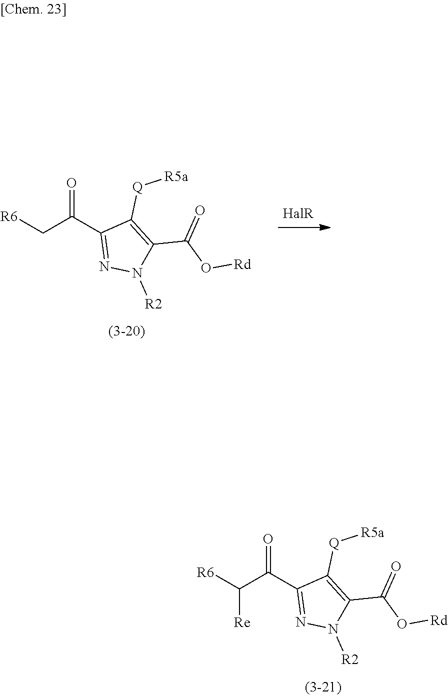

In the formula, HalR represents a halogenating agent, and R2, R5a, Q, R6, Rd, and Re have the same meanings as above.

A production method Q is a production method for obtaining a compound represented by Formula (3-21) in which Re represents a halogen atom, the production method comprising reacting a compound represented by Formula (3-20) with a halogenation agent (HalR) in a solvent.

The production method Q can be performed in accordance with the production method O by using the compound represented by Formula (3-18) instead of the compound represented by Formula (3-20) in the production method O.

### Production Method R

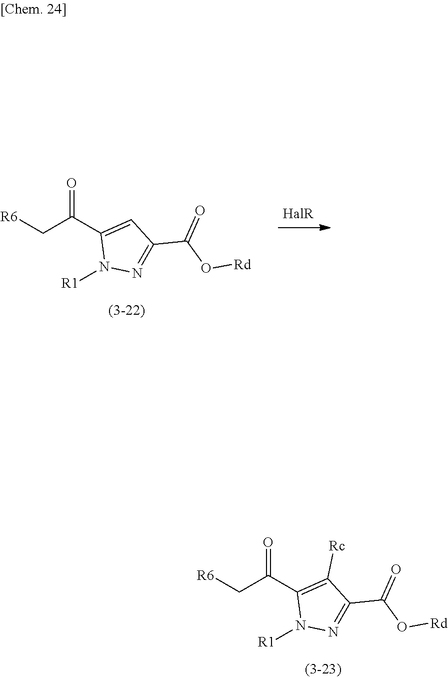

In the formula, R1, R6, Re, Rd, and HalR have the same meanings as above.

A production method R is a production method for obtaining a compound represented by Formula (3-23) in which Rc represents a halogen atom, the production method comprising reacting a compound represented by Formula (3-22) with a halogenation agent (HalR) in a solvent.

The production method R can be performed in accordance with the production method C by using the compound represented by Formula (3-3) instead of the compound represented by Formula (3-22) in the production method C.

### Production Method S

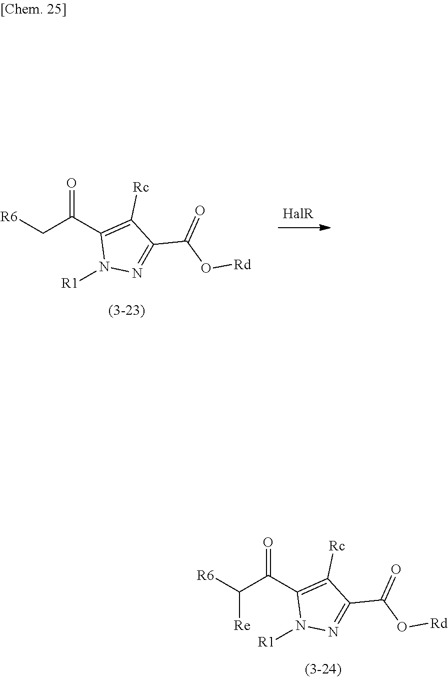

In the formula, R1, R6, Rc, Rd, Re, and HalR have the same meanings as above.

A production method S is a production method for obtaining a compound represented by Formula (3-24) in which Re represents a halogen atom, the production method comprising reacting a compound represented by Formula (3-23) with a halogenation agent (HalR) in a solvent.

The production method S can be performed in accordance with the production method O by using the compound represented by Formula (3-18) instead of the compound represented by Formula (3-23) in the production method O.

### Production Method T

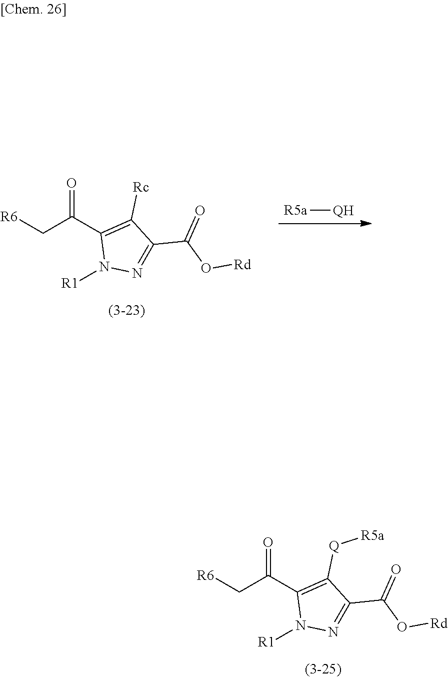

In the formula, R1, R5a, Q, R6, Re, and Rd have the same meanings as above.

A production method T is a method for synthesizing a compound represented by Formula (3-25), the production method comprising reacting a compound represented by Formula (3-23) with R5a-QH (where R5a has the same meaning as above) in a solvent in the presence of a base.

The production method T can be performed in accordance with the production method D by using the compound represented by Formula (3-4) instead of the compound represented by Formula (3-23) in the production method D.

### Production Method U

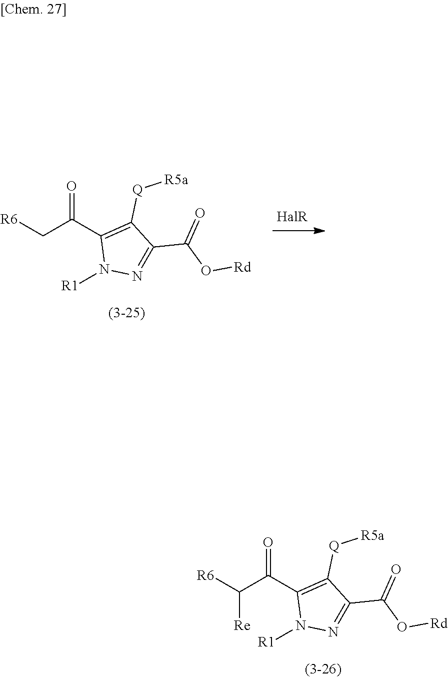

In the formula, HalR represents a halogenating agent, and R1, R5a, Q, R6, Rd, and Re have the same meanings as above.

A production method U is a production method for obtaining a compound represented by Formula (3-26) in which Re represents a halogen atom, the production method comprising reacting a compound represented by Formula (3-25) with a halogenation agent (HalR) in a solvent.

The production method U can be performed in accordance with the production method O by using the compound represented by Formula (3-18) instead of the compound represented by Formula (3-25) in the production method O.

### Production Method V

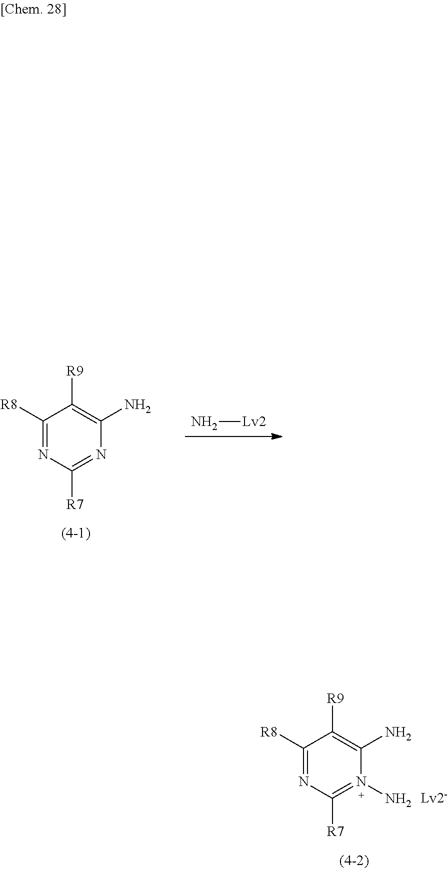

In the formula, R7 represents a hydrogen atom, a hydroxyl group, a cyano group, a halogen atom, a C1 to C6 alkyl group optionally substituted with a substituent A, a C1 to C6 haloalkyl group, a C3 to C8 cycloalkyl group optionally substituted with a substituent B, a C2 to C6 alkenyl group optionally substituted with a substituent A, a C2 to C6 haloalkenyl group, a C2 to C6 alkynyl group optionally substituted with a substituent A, a C2 to C6 haloalkynyl group, a C1 to C6 alkoxy group optionally substituted with a substituent A, a C1 to C6 haloalkoxy group, a C3 to C8 cycloalkoxy group optionally substituted with a substituent A, or Rx4S(O)p- (where Rx4 represents a hydroxyl group, a halogen atom, a C1 to C6 alkyl group optionally substituted with a substituent A, a C1 to C6 haloalkyl group, a C3 to C8 cycloalkyl group optionally substituted with a substituent B, a C2 to C6 alkenyl group optionally substituted with a substituent A, a C2 to C6 haloalkenyl group, a C2 to C6 alkynyl group optionally substituted with a substituent A, a C2 to C6 haloalkynyl group, or a phenyl group optionally substituted with 0 to 5 substituents B, and p represents an integer of 0, 1, or 2),
R8 represents a hydrogen atom, a hydroxyl group, a cyano group, a halogen atom, a C1 to C6 alkyl group optionally substituted with a substituent A, a C1 to C6 haloalkyl group, a C3 to C8 cycloalkyl group optionally substituted with a substituent B, a C1 to C6 alkoxy group optionally substituted with a substituent A, a C1 to C6 haloalkoxy group, a C3 to C8 cycloalkoxy group optionally substituted with a substituent A, or Rx4S(O)p- (where Rx4 and p have the same meanings as above),
R9 represents a hydrogen atom, a hydroxyl group, a cyano group, a halogen atom, a C1 to C6 alkyl group optionally substituted with a substituent A, a C1 to C6 haloalkyl group, a C3 to C8 cycloalkyl group optionally substituted with a substituent B, a C1 to C6 alkoxy group optionally substituted with a substituent A, a C1 to C6 haloalkoxy group, a C3 to C8 cycloalkoxy group optionally substituted with a substituent A, or Rx4S(O)p- (where Rx4 and p have the same meanings as above), and
Lv2 represents a leaving group such as a sulfonate group, a mesitylsulfonate group, a 2,4-dinitrophenoxy group, or a diphenylphosphinate group.

A production method V is a production method for obtaining a compound represented by Formula (4-2), the production method comprising reacting a compound represented by Formula (4-1) with an aminating agent (NH₂—Lv2) in a solvent.

Examples of the aminating agent used in the present reaction include hydroxylamine-O-sulfonic acid, O-(diphenylphosphinyl)hydroxylamine, (0-(mesitylsulfonyl)hydroxylamine), and O-(2,4-dinitrophenyl)hydroxylamine.

The amount of aminating agent used in the present reaction may be 1 equivalent or more with respect to the compound represented by Formula (4-1), is not particularly limited as long as the intended reaction proceeds, and is preferably 1 equivalent or more and 10 equivalents or less, and more preferably 1 equivalent or more and 5 equivalents or less.

The solvent used in the present reaction is not particularly limited as long as the intended reaction proceeds, and examples thereof include acidic solvents such as sulfuric acid, acetic acid, trifluoroacetic acid, methanesulfonic acid, and trifluoromethanesulfonic acid, ether-based solvents such as diethyl ether, diisopropyl ether, methyl-t-butyl ether, dimethoxyethane, tetrahydrofuran, and dioxane, alcohol-based solvents such as methanol, ethanol, and isopropanol, benzene-based solvents such as benzene, toluene, xylene, mesitylene, chlorobenzene, and dichlorobenzene, ester-based solvents such as ethyl acetate, isopropyl acetate, and butyl acetate, nitrile-based solvents such as acetonitrile, amide-based solvents such as N-methylpyrrolidone, N,N-dimethylformamide, and N,N-dimethylacetamide, urea-based solvents such as 1,3-dimethyl-2 imidazolidinone, and halogen-based solvents such as dichloromethane, dichloroethane, chloroform, and carbon tetrachloride. These solvents can be used alone or as a mixture of two or more kinds thereof in an arbitrary ratio.

The amount of solvent used in the present reaction is not particularly limited as long as the desired reaction proceeds, and is usually 1 to 200 times by weight with respect to the compound represented by Formula (4-1).

The temperature for performing the present reaction is not particularly limited as long as the intended reaction proceeds, but is usually 0° C. or higher and 150° C. or lower or below a boiling point of the solvent.

As the post-treatment of the reaction, a liquid separation operation can be performed by adding water or an appropriate aqueous solution to the reaction mixture. When an aqueous solution is used, an acidic aqueous solution in which hydrochloric acid, sulfuric acid, ammonium chloride, or the like is dissolved, an alkaline aqueous solution in which potassium hydroxide, sodium hydroxide, sodium carbonate, potassium carbonate, sodium hydrogen carbonate, potassium hydrogen carbonate, or the like is dissolved, an aqueous solution containing salts containing sulfur atoms such as sodium thiosulfate and sodium sulfite, a saline solution, or the like can be arbitrarily used. In the liquid separation operation, it is possible to add a solvent that is not compatible with water, such as a benzene-based solvent such as toluene, xylene, benzene, chlorobenzene, or dichlorobenzene, an ester-based solvent such as ethyl acetate, isopropyl acetate, or butyl acetate, an ether-based solvent such as diethyl ether, diisopropyl ether, or methyl-t-butyl ether, a halogen-based solvent such as dichloromethane, dichloroethane, chloroform, or carbon tetrachloride, or a hydrocarbon-based solvent such as hexane, heptane, cyclohexane, or methylcyclohexane, as necessary. In addition, these solvents can be used alone, or two or more kinds thereof can be mixed at an arbitrary ratio. The number of times of liquid separation is not particularly limited, and the liquid separation can be performed according to the intended purity and yield.

In the reaction mixture containing the compound represented by Formula (4-2) obtained above, moisture can be removed with a desiccant such as sodium sulfate or magnesium sulfate, but it is not essential.

The reaction mixture containing the compound represented by Formula (4-2) obtained above can be subjected to solvent distillation under reduced pressure as long as the compounds are not decomposed.

A reaction mixture containing the compound represented by Formula (4-2) obtained after solvent distillation can be purified by washing, reprecipitation, recrystallization, column chromatography, or the like with an appropriate solvent. It may be appropriately set according to the intended purity.

### Production Method W

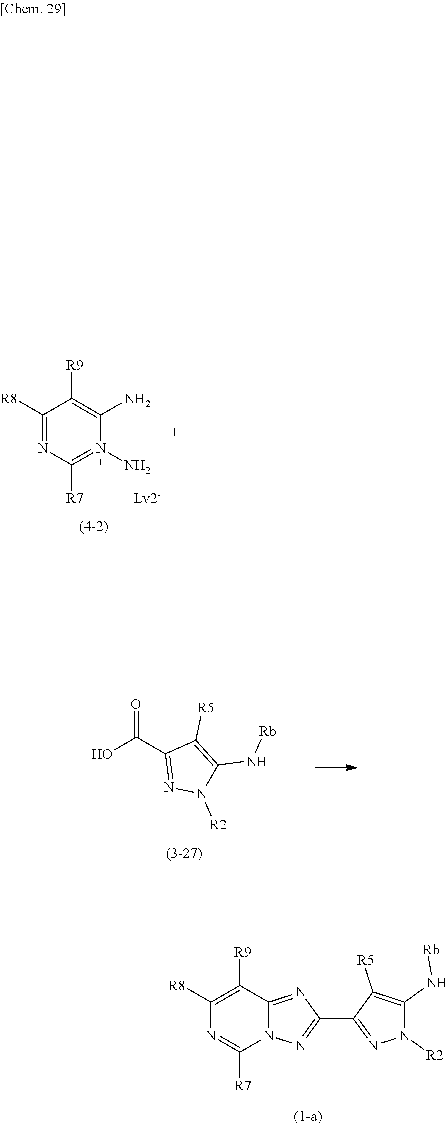

R5 in the formula represents a cyano group, a halogen atom, a C1 to C6 alkoxy group optionally substituted with a substituent A, a C1 to C6 haloalkoxy group, a C3 to C8 cycloalkoxy group optionally substituted with a substituent A, a C2 to C6 alkenyl group optionally substituted with a substituent A, a C2 to C6 haloalkenyl group, a C2 to C6 alkynyl group optionally substituted with a substituent A, a C2 to C6 haloalkynyl group, or Rx4S(O)p- (where Rx4 and p have the same meanings as above), and R2, R7, R8, R9, Rb, and Lv2 have the same meanings as above.

A production method W is a production method for obtaining a compound represented by Formula (1-a) of the present invention, the production method comprising reacting a compound represented by Formula (4-2) with a compound represented by Formula (3-27) in a solvent.

In the present reaction, a pyrazole carboxylic acid represented by Formula (3-27) can be converted into a carboxylic acid halide, and then reacted with the compound represented by Formula (4-2) in the presence of a base. In addition, the pyrazole carboxylic acid represented by Formula (3-27) and the compound represented by Formula (4-2) can be reacted in the presence of a base using a condensing agent.

First, a reaction for synthesizing the compound represented by Formula (3-27) into a carboxylic acid halide will be described.

Examples of the halogenating agent for synthesizing the carboxylic acid halide include thionyl chloride, oxalyl chloride, phosphoryl chloride, sulfuryl chloride, phosphorus trichloride, phosphorus pentachloride, and phosphorus tribromide.

The amount of halogenating agent used in the present reaction may be 1 equivalent or more with respect to the compound represented by Formula (3-27), is not particularly limited as long as the intended reaction proceeds, and is preferably 1 equivalent or more and 10 equivalents or less.

When a carboxylic acid halide is synthesized, N,N-dimethylformamide can coexist as a catalyst. The amount of catalyst used in the present reaction is not particularly limited as long as the intended reaction proceeds, and is preferably 0.01 equivalents or more and 10 equivalents or less with respect to the compound represented by Formula (3-27).

The solvent used in the present reaction is not particularly limited as long as the intended reaction proceeds, and examples thereof include ether-based solvents such as diethyl ether, diisopropyl ether, methyl-t-butyl ether, dimethoxyethane, tetrahydrofuran, and dioxane, benzene-based solvents such as benzene, toluene, xylene, mesitylene, chlorobenzene, and dichlorobenzene, ester-based solvents such as ethyl acetate, isopropyl acetate, and butyl acetate, amide-based solvents such as N-methylpyrrolidone, N,N-dimethylformamide, and N,N-dimethylacetamide, urea-based solvents such as 1,3-dimethyl-2 imidazolidinone, and halogen-based solvents such as dichloromethane, dichloroethane, chloroform, and carbon tetrachloride. These solvents can be used alone or as a mixture of two or more kinds thereof in an arbitrary ratio.

The amount of solvent used in the present reaction is not particularly limited as long as the desired reaction proceeds, and is usually 1 to 200 times by weight with respect to the compound represented by Formula (3-27).

The temperature for performing the present reaction is not particularly limited as long as the intended reaction proceeds, but is usually −10° C. or higher and 150° C. or lower or below a boiling point of the solvent.

The reaction mixture containing the carboxylic acid halide obtained above can be dehydrated with a drying agent such as sodium sulfate or magnesium sulfate, but it is not essential.

The reaction mixture containing the carboxylic acid halide obtained above can be subjected to solvent distillation under reduced pressure as long as the compounds are not decomposed.

A reaction mixture containing the carboxylic acid halide obtained after solvent distillation can be purified by washing, reprecipitation, recrystallization, column chromatography, or the like with an appropriate solvent. It may be appropriately set according to the intended purity.

Next, the reaction between the compound represented by Formula (4-2) and the carboxylic acid halide obtained above in the presence of a base will be described.

The amount of carboxylic acid halide used in the present reaction is not particularly limited as long as the intended reaction proceeds, and is preferably 1 equivalent or more and 10 equivalents or less with respect to the compound represented by Formula (4-2).

Next, the reaction in the presence of a base will be described.

Examples of the base used in the present reaction include inorganic bases such as sodium hydroxide, potassium hydroxide, sodium carbonate, potassium carbonate, cesium carbonate, and sodium hydride, organic bases such as triethylamine, tributylamine, diisopropylethylamine, 1,8-diazabicyclo[5.4.0]-7-undecene, and 1,4-diazabicyclo[2.2.2]octane, metal hydrides such as sodium hydride, organic lithiums such as methyllithium, butyllithium, sec-butyllithium, t-butyllithium, and hexyllithium, and metal amides such as lithium diisopropylamide, hexamethyldisilazane lithium, hexamethyldisilazane sodium, and hexamethyldisilazane potassium.

The base used in the present reaction may be 1 equivalent or more with respect to the compound represented by Formula (3-27), is not particularly limited as long as the intended reaction proceeds, and is preferably 1 equivalent or more and 30 equivalents or less.

In the present reaction, a catalyst can be used as an activator. Examples of the catalyst include 4-dimethylaminopyridine and 4-pyrrolidinopyridine.

The amount of catalyst used in the present reaction is not particularly limited as long as the intended reaction proceeds, and is preferably 0.01 equivalents or more and 10 equivalents or less with respect to the compound represented by Formula (4-2).

The solvent used in the present reaction is not particularly limited as long as the intended reaction proceeds, and examples thereof include acidic solvents such as sulfuric acid, acetic acid, trifluoroacetic acid, methanesulfonic acid, and trifluoromethanesulfonic acid, ether-based solvents such as diethyl ether, diisopropyl ether, methyl-t-butyl ether, dimethoxyethane, tetrahydrofuran, and dioxane, benzene-based solvents such as benzene, toluene, xylene, mesitylene, chlorobenzene, and dichlorobenzene, ester-based solvents such as ethyl acetate, isopropyl acetate, and butyl acetate, amide-based solvents such as N-methylpyrrolidone, N,N-dimethylformamide, and N,N-dimethylacetamide, urea-based solvents such as 1,3-dimethyl-2 imidazolidinone, and halogen-based solvents such as dichloromethane, dichloroethane, chloroform, and carbon tetrachloride. These solvents can be used alone or as a mixture of two or more kinds thereof in an arbitrary ratio.

The amount of solvent used in the present reaction is not particularly limited as long as the desired reaction proceeds, and is usually 1 to 200 times by weight with respect to the compound represented by Formula (4-2).

The temperature for performing the present reaction is not particularly limited as long as the intended reaction proceeds, but is usually −10° C. or higher and 150° C. or lower or below a boiling point of the solvent.

As the post-treatment of the reaction, a liquid separation operation can be performed by adding water or an appropriate aqueous solution to the reaction mixture. When an aqueous solution is used, an acidic aqueous solution in which hydrochloric acid, sulfuric acid, ammonium chloride, or the like is dissolved, an alkaline aqueous solution in which potassium hydroxide, sodium hydroxide, sodium carbonate, potassium carbonate, sodium hydrogen carbonate, potassium hydrogen carbonate, or the like is dissolved, a saline solution, or the like can be arbitrarily used. In the liquid separation operation, it is possible to add a solvent that is not compatible with water, such as a benzene-based solvent such as toluene, xylene, benzene, chlorobenzene, or dichlorobenzene, an ester-based solvent such as ethyl acetate, isopropyl acetate, or butyl acetate, an ether-based solvent such as diethyl ether, diisopropyl ether, or methyl-t-butyl ether, a halogen-based solvent such as dichloromethane, dichloroethane, chloroform, or carbon tetrachloride, or a hydrocarbon-based solvent such as hexane, heptane, cyclohexane, or methylcyclohexane, as necessary. In addition, these solvents can be used alone, or two or more kinds thereof can be mixed at an arbitrary ratio. The number of times of liquid separation is not particularly limited, and the liquid separation can be performed according to the intended purity and yield. In addition, it is also possible to remove insoluble matters by performing a filtration operation, but it is not essential.

In the reaction mixture containing the compound represented by Formula (1-a) obtained above, moisture can be removed with a desiccant such as sodium sulfate or magnesium sulfate, but it is not essential.

The reaction mixture containing the compound represented by Formula (1-a) obtained above can be subjected to solvent distillation under reduced pressure as long as the compounds are not decomposed.

A reaction mixture containing the compound represented by Formula (1-a) obtained after solvent distillation can be purified by washing, reprecipitation, recrystallization, column chromatography, or the like with an appropriate solvent.

Next, the method for reacting the compound represented by Formula (3-27) with the compound represented by Formula (4-2) in the presence of a base using a condensing agent will be described.

The amount of Formula (3-27) used in the present reaction is not particularly limited as long as the intended reaction proceeds, and is preferably 1 equivalent or more and 10 equivalents or less with respect to the compound represented by Formula (4-2).

Examples of the condensing agent used in the present reaction include a carbodiimide condensing agent such as 1-ethyl-3-(3-dimethylaminopropyl)carbodiimide hydrochloride, 1-[3-(dimethylamino)propyl]-3-ethylcarbodiimide, N,N′-dicyclohexylcarbodiimide, or N,N′-diisopropylcarbodiimide, an imidazole dehydration condensation agent such as N,N′-carbonyldiimidazole or 1,1′-carbonyldi(1,2,4-triazole), a triazine condensing agent such as 4-(4,6-dimethoxy-1,3,5-triazin-2-yl)-4-methylmorpholinium chloride, a phosphonium dehydration condensation agent such as 1H-benzotriazol-1-yloxytris(dimethylamino)phosphonium hexafluorophosphate, 1H-benzotriazol-1-yloxytripyrrolidinophosphonium hexafluorophosphate, or 1-[bis(dimethylamino)methylene]-1H-1,2,3-triazolo[4,5-b]pyridinium 3-oxide hexafluorophosphate, an uronium condensing agent such as {{[(1-cyano-2-ethoxy-2-oxoethylidene)amino]oxy}-4-morphoninomethylene}dimethylammonium hexafluorophosphate, O-(benzotriazol-1-yl)-N,N,N′,N′,-tetramethyluronium hexafluorophosphate, or O-(7-azabenzotriazol-1-yl)-N,N,N′,N′,-tetramethyluronium hexafluorophosphate, a halouronium condensing agent such as 2-chloro-1,3-dimethylimidazolinium hexafluorophosphate or 1-(chloro-1-pyrrolidinylmethylene)pyrrolidinium hexafluorophosphate, and 2-chloro-1-methylpyridinium iodide.

The amount of condensing agent used in the present reaction is not particularly limited as long as the intended reaction proceeds, and is preferably 1 equivalent or more and 10 equivalents or less with respect to the compound represented by Formula (3-27).

In the present reaction, an additive can be used together with a condensing agent. Examples of the additive include 1-hydroxybenzotriazole, 1-hydroxy-7-azabenzotriazole, N-hydroxysuccinimide, N,N′-disuccinimidyl carbonate, and dimethylaminopyridine.

The amount of additive used in the present reaction is not particularly limited as long as the intended reaction proceeds, and is preferably 0.1 equivalents or more and 10 equivalents or less with respect to the compound represented by Formula (3-27).

Examples of the base used in the present reaction include inorganic bases such as sodium hydroxide, potassium hydroxide, sodium carbonate, potassium carbonate, cesium carbonate, and sodium hydride, organic bases such as triethylamine, tributylamine, diisopropylethylamine, 1,8-diazabicyclo[5.4.0]-7-undecene, and 1,4-diazabicyclo[2.2.2]octane, metal hydrides such as sodium hydride, organic lithiums such as methyllithium, butyllithium, sec-butyllithium, t-butyllithium, and hexyllithium, and metal amides such as lithium diisopropylamide, hexamethyldisilazane lithium, hexamethyldisilazane sodium, and hexamethyldisilazane potassium.

The base used in the present reaction may be 1 equivalent or more with respect to the compound represented by Formula (3-27), is not particularly limited as long as the intended reaction proceeds, and is preferably 1 equivalent or more and 30 equivalents or less.

The solvent used in the present reaction is not particularly limited as long as the intended reaction proceeds, and examples thereof include ether-based solvents such as diethyl ether, diisopropyl ether, methyl-t-butyl ether, dimethoxyethane, tetrahydrofuran, and dioxane, benzene-based solvents such as benzene, toluene, xylene, mesitylene, chlorobenzene, and dichlorobenzene, ester-based solvents such as ethyl acetate, isopropyl acetate, and butyl acetate, nitrile-based solvents such as acetonitrile, amide-based solvents such as N-methylpyrrolidone, N,N-dimethylformamide, and N,N-dimethylacetamide, urea-based solvents such as 1,3-dimethyl-2 imidazolidinone, halogen-based solvents such as dichloromethane, dichloroethane, chloroform, and carbon tetrachloride, and organic base solvents such as triethylamine, tributylamine, diisopropylethylamine, pyridine, and 2,6-lutidine. These solvents can be used alone or as a mixture of two or more kinds thereof in an arbitrary ratio.

The amount of solvent used in the present reaction is not particularly limited as long as the desired reaction proceeds, and is usually 1 to 200 times by weight with respect to the compound represented by Formula (3-27).

The temperature for performing the present reaction is not particularly limited as long as the intended reaction proceeds, but is usually −10° C. or higher and 150° C. or lower or below a boiling point of the solvent.

As the post-treatment of the reaction, a liquid separation operation can be performed by adding water or an appropriate aqueous solution to the reaction mixture. When an aqueous solution is used, an acidic aqueous solution in which hydrochloric acid, sulfuric acid, ammonium chloride, or the like is dissolved, an alkaline aqueous solution in which potassium hydroxide, sodium hydroxide, sodium carbonate, potassium carbonate, sodium hydrogen carbonate, potassium hydrogen carbonate, or the like is dissolved, a saline solution, or the like can be arbitrarily used. In the liquid separation operation, it is possible to add a solvent that is not compatible with water, such as a benzene-based solvent such as toluene, xylene, benzene, chlorobenzene, or dichlorobenzene, an ester-based solvent such as ethyl acetate, isopropyl acetate, or butyl acetate, an ether-based solvent such as diethyl ether, diisopropyl ether, or methyl-t-butyl ether, a halogen-based solvent such as dichloromethane, dichloroethane, chloroform, or carbon tetrachloride, or a hydrocarbon-based solvent such as hexane, heptane, cyclohexane, or methylcyclohexane, as necessary. In addition, these solvents can be used alone, or two or more kinds thereof can be mixed at an arbitrary ratio. The number of times of liquid separation is not particularly limited, and the liquid separation can be performed according to the intended purity and yield. In addition, it is also possible to remove insoluble matters by performing a filtration operation, but it is not essential.

In the reaction mixture containing the compound represented by Formula (1-a) obtained above, moisture can be removed with a desiccant such as sodium sulfate or magnesium sulfate, but it is not essential.

The reaction mixture containing the compound represented by Formula (1-a) obtained above can be subjected to solvent distillation under reduced pressure as long as the compounds are not decomposed.

A reaction mixture containing the compound represented by Formula (1-a) obtained after solvent distillation can be purified by washing, reprecipitation, recrystallization, column chromatography, or the like with an appropriate solvent. It may be appropriately set according to the intended purity.

### Production Method X

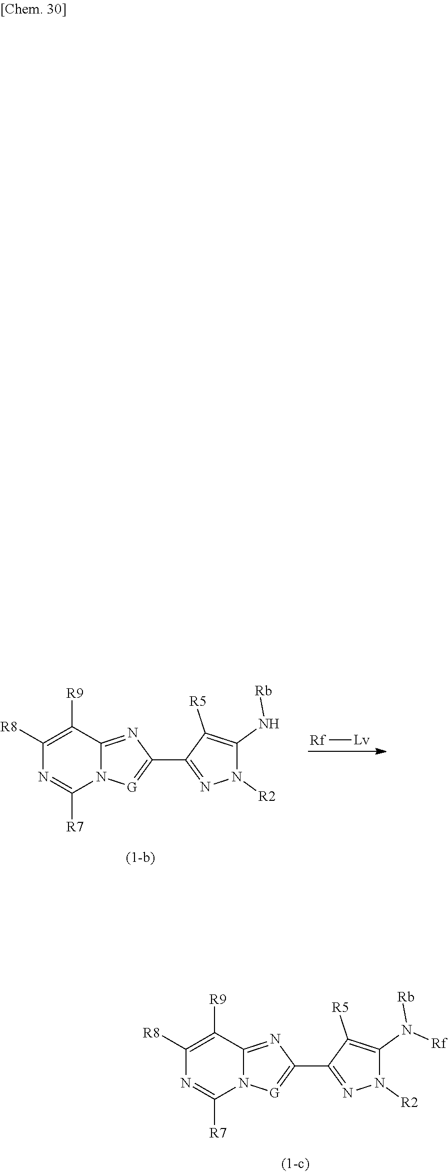

In the formula, G represents C—R6 or a nitrogen atom, R6 represents a hydrogen atom, a cyano group, a halogen atom, a C1 to C6 alkyl group optionally substituted with a substituent A, a C1 to C6 haloalkyl group, a C3 to C8 cycloalkyl group optionally substituted with a substituent B, a C2 to C6 alkenyl group optionally substituted with a substituent A, a C2 to C6 haloalkenyl group, a C2 to C6 alkynyl group optionally substituted with a substituent A, or a C2 to C6 haloalkynyl group, Rf represents a C1 to C6 alkyl group optionally substituted with a substituent A, a C1 to C6 haloalkyl group, a C3 to C8 cycloalkyl group optionally substituted with a substituent B, a C2 to C6 alkenyl group optionally substituted with a substituent A, a C2 to C6 haloalkenyl group, a C2 to C6 alkynyl group optionally substituted with a substituent A, a C2 to C6 haloalkynyl group, Rx1Rx2NC(═O)— (where Rx1 and Rx2 have the same meanings as above), Rx3C(═O)— (where Rx3 has the same meaning as above), Rx3OC(═O)— (where Rx3 has the same meaning as above), or Rx4S(O)p- (where Rx4 and p have the same meanings as above), and R2, R5, R7, R8, R9, Rb, and Lv have the same meanings as above.

A production method X is a method for obtaining a compound represented by Formula (1-c) including the compound of the present invention, the production method comprising reacting a compound represented by Formula (1-b) with Rf-Lv in a solvent in the presence of a base.

Rf-Lv used in the present reaction can be obtained as a commercially available product or produced by a known method. When a compound in which Rf is Rx3C(═O)— (where Rx3 has the same meaning as above) is used, Rf-Lv may use an acid anhydride.

The amount of Rf-Lv used in the present reaction may be 1 equivalent or more with respect to the compound represented by Formula (1-b), is not particularly limited as long as the intended reaction proceeds, and is preferably 1 equivalent or more and 10 equivalents or less.

Examples of the base used in the present reaction include inorganic bases such as sodium hydroxide, potassium hydroxide, sodium carbonate, potassium carbonate, cesium carbonate, and sodium hydride, organic bases such as triethylamine, tributylamine, diisopropylethylamine, 1,8-diazabicyclo[5.4.0]-7-undecene, 1,4-diazabicyclo[2.2.2]octane, and dimethylaminopyridine, metal hydrides such as sodium hydride, organic lithiums such as methyllithium, butyllithium, sec-butyllithium, t-butyllithium, and hexyllithium, and metal amides such as lithium diisopropylamide, hexamethyldisilazane lithium, hexamethyldisilazane sodium, and hexamethyldisilazane potassium.

The amount of base used in the present reaction may be 1 equivalent or more with respect to the compound represented by Formula (1-b), is not particularly limited as long as the intended reaction proceeds, and is preferably 1 equivalent or more and 10 equivalents or less.

The solvent used in the present reaction is not particularly limited as long as the intended reaction proceeds, and examples thereof include ether-based solvents such as diethyl ether, diisopropyl ether, methyl-t-butyl ether, dimethoxyethane, tetrahydrofuran, and dioxane, benzene-based solvents such as benzene, toluene, xylene, mesitylene, chlorobenzene, and dichlorobenzene, ester-based solvents such as ethyl acetate, isopropyl acetate, and butyl acetate, nitrile-based solvents such as acetonitrile, amide-based solvents such as N-methylpyrrolidone, N,N-dimethylformamide, and N,N-dimethylacetamide, urea-based solvents such as 1,3-dimethyl-2 imidazolidinone, and halogen-based solvents such as dichloromethane, dichloroethane, chloroform, and carbon tetrachloride. These solvents can be used alone or as a mixture of two or more kinds thereof in an arbitrary ratio.

The amount of solvent used in the present reaction is not particularly limited as long as the desired reaction proceeds, and is usually 3 to 200 times by weight with respect to the compound represented by Formula (1-b).

The temperature for performing the present reaction is not particularly limited as long as the intended reaction proceeds, but is usually −10° C. or higher and 180° C. or lower or below a boiling point of the solvent.

As the post-treatment of the reaction, a liquid separation operation can be performed by adding water or an appropriate aqueous solution to the reaction mixture. When an aqueous solution is used, an acidic aqueous solution in which hydrochloric acid, sulfuric acid, ammonium chloride, or the like is dissolved, an alkaline aqueous solution in which potassium hydroxide, sodium hydroxide, sodium carbonate, potassium carbonate, sodium hydrogen carbonate, potassium hydrogen carbonate, or the like is dissolved, an aqueous solution containing salts containing sulfur atoms such as sodium thiosulfate and sodium sulfite, a saline solution, or the like can be arbitrarily used. In the liquid separation operation, it is possible to add a solvent that is not compatible with water, such as a benzene-based solvent such as toluene, xylene, benzene, chlorobenzene, or dichlorobenzene, an ester-based solvent such as ethyl acetate, isopropyl acetate, or butyl acetate, an ether-based solvent such as diethyl ether, diisopropyl ether, or methyl-t-butyl ether, a halogen-based solvent such as dichloromethane, dichloroethane, or chloroform, or a hydrocarbon-based solvent such as hexane, heptane, cyclohexane, or methylcyclohexane, as necessary. In addition, these solvents can be used alone, or two or more kinds thereof can be mixed at an arbitrary ratio. The number of times of liquid separation is not particularly limited, and the liquid separation can be performed according to the intended purity and yield.

In the reaction mixture containing the compound represented by Formula (1-c) obtained above, moisture can be removed with a desiccant such as sodium sulfate or magnesium sulfate, but it is not essential.

The reaction mixture containing the compound represented by Formula (1-c) obtained above can be subjected to solvent distillation under reduced pressure as long as the compounds are not decomposed.

A reaction mixture containing the compound represented by Formula (1-c) obtained after solvent distillation can be purified by washing, reprecipitation, recrystallization, column chromatography, or the like with an appropriate solvent. It may be appropriately set according to the intended purity.

### Production Method Y

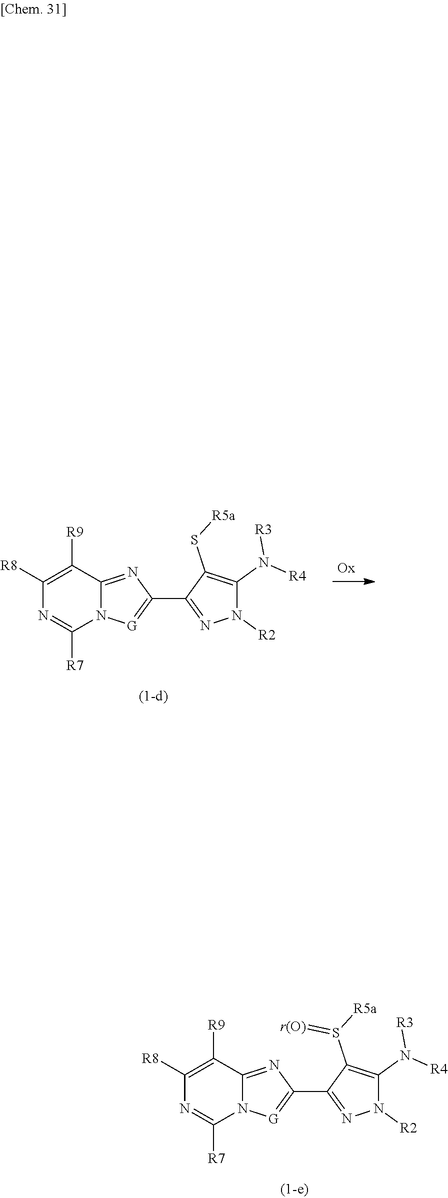

In the formula, G, R2, R3, R4, R5a, R6, R7, R8, R9, r, and Ox have the same meanings as above.

A production method Y is a method for obtaining a compound represented by Formula (1-e), the production method comprising reacting a compound represented by Formula (1-d) with an oxidizing agent (Ox) in a solvent.

The production method Y can be performed in accordance with the production method F by using the compound represented by Formula (3-6) instead of the compound represented by Formula (1-d) in the production method F.

### Production Method Z

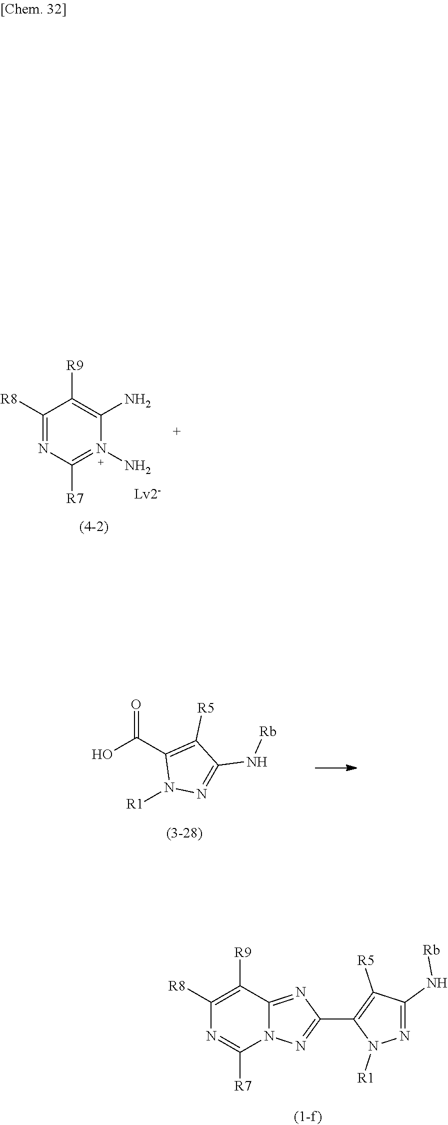

In the formula, R1, R5, R7, R8, R9, and Rb have the same meanings as above.

A production method Z is a production method for obtaining a compound represented by Formula (1-f) of the present invention, the production method comprising reacting a compound represented by Formula (4-2) with a compound represented by Formula (3-28) in a solvent.

In the present reaction, a pyrazole carboxylic acid represented by Formula (3-28) can be converted into a carboxylic acid halide, and then reacted with the compound represented by Formula (4-2) in the presence of a base. In addition, the pyrazole carboxylic acid represented by Formula (3-28) and the compound represented by Formula (4-2) can be reacted in the presence of a base using a condensing agent.

The production method Z can be performed in accordance with the production method W by using the compound represented by Formula (3-27) instead of the compound represented by Formula (3-28) in the production method W.

### Production Method AA

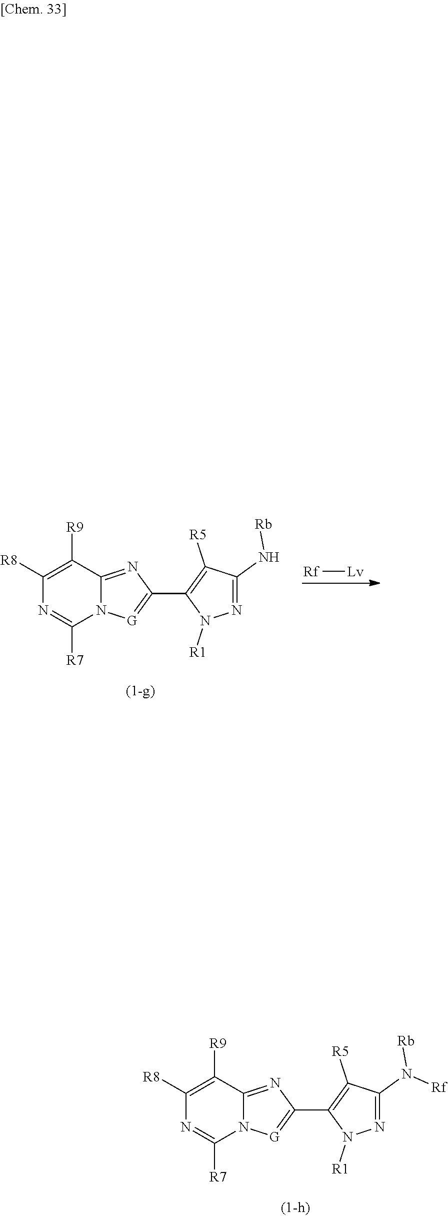

In the formula, G, R1, R5, R6, R7, R8, R9, Rb, Rf, and Lv have the same meanings as above.

A production method AA is a method for obtaining a compound represented by Formula (1-h) including the compound of the present invention, the production method comprising reacting a compound represented by Formula (1-g) with Rf-Lv in a solvent. The production method AA can be performed in accordance with the production method X by using the compound represented by Formula (1-b) instead of the compound represented by Formula (1-g) in the production method X.

### Production Method AB

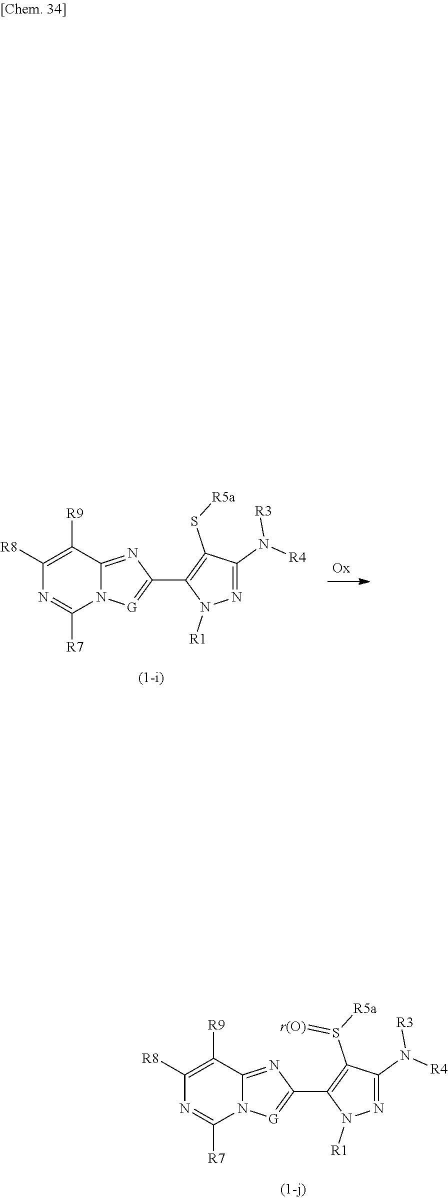

In the formula, G, R1, R3, R4, R5a, R7, R8, R9, r, and Ox have the same meanings as above.

A production method AB is a method for obtaining a compound represented by Formula (1-j), the production method comprising reacting a compound represented by Formula (1-i) with an oxidizing agent (Ox) in a solvent.

The production method AB can be performed in accordance with the production method F by using the compound represented by Formula (3-6) instead of the compound represented by Formula (1-i) in the production method F.

### Production Method AC

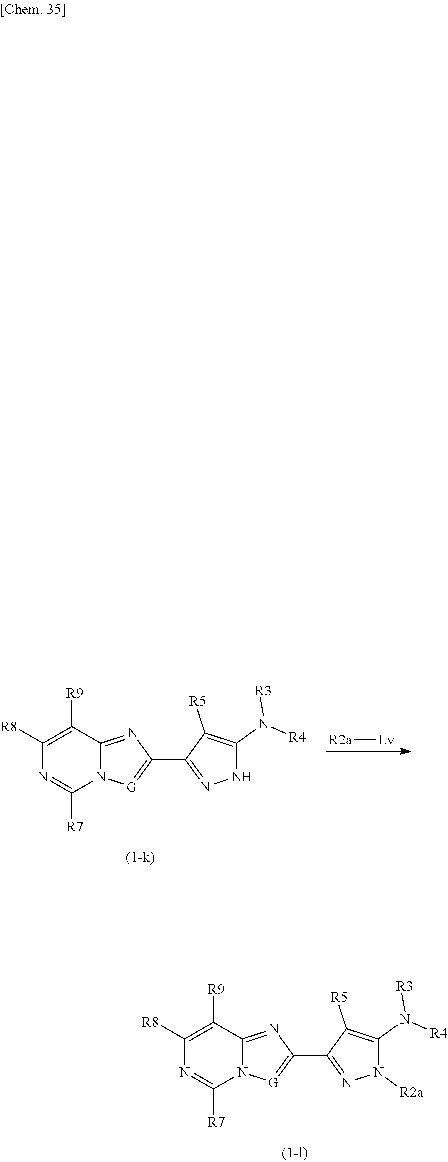

In the formula, R2a represents a C1 to C6 alkyl group optionally substituted with a substituent A, a C1 to C6 haloalkyl group, a C3 to C8 cycloalkyl group optionally substituted with a substituent B, a C2 to C6 alkenyl group optionally substituted with a substituent A, a C2 to C6 haloalkenyl group, a C2 to C6 alkynyl group optionally substituted with a substituent A, a C2 to C6 haloalkynyl group, a phenyl group optionally substituted with 0 to 5 substituents B, a 5- or 6-membered aromatic heterocyclic group optionally substituted with 0 to 4 substituents B, Rx1Rx2NC(═O)— (where Rx1 and Rx2 have the same meanings as above), Rx3C(═O)— (where Rx3 has the same meaning as above), Rx3OC(═O)— (where Rx3 has the same meaning as above), Rx4S(O)p- (where Rx4 and p have the same meanings as above), or a 3- to 6-membered ring group containing one or two oxygen atoms, and G, R3, R4, R5, R6, R7, R8, R9, and Lv have the same meanings as above.

A production method AC is a method for obtaining a compound represented by Formula (1-1), the production method comprising reacting a compound represented by Formula (1-k) with R2a-Lv in a solvent in the presence of a base.

The compound represented by Formula (1-k) includes a tautomer represented by Formula (1-m)

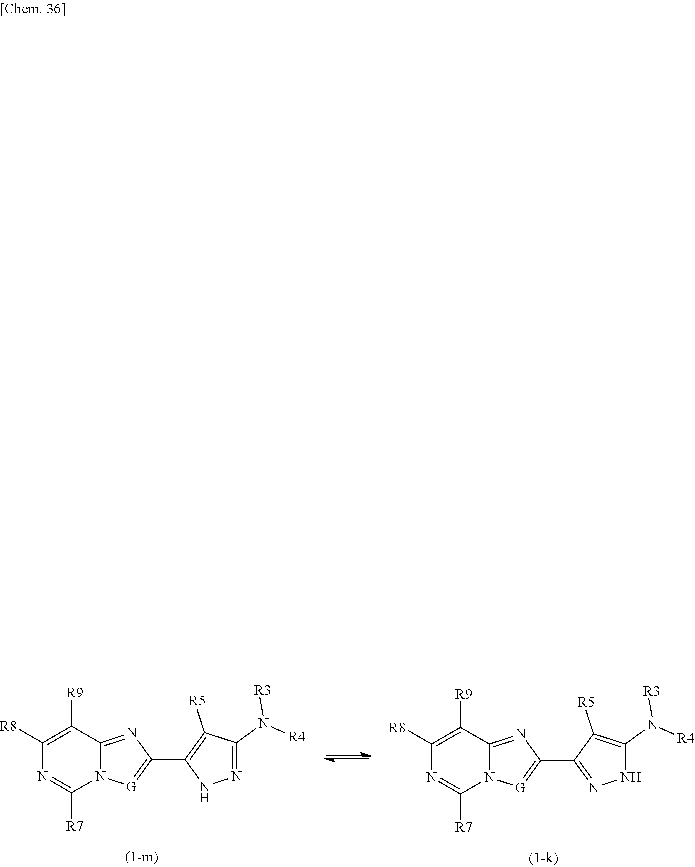

(in the formula, G, R3, R4, R5, R6, R7, R8, and R9 have the same meanings as above).

The compound represented by Formula (1-k) can be handled in the same manner as the compound represented Formula (1-m), and the production method AC can be applied. It may be a mixture of the compound represented by Formula (1-k) and the compound represented by Formula (1-m), and these isomers may be used alone or as a mixture in an arbitrary ratio.

R2a-Lv used in the present reaction can be obtained as a commercially available product or produced by a known method.

The amount of R2a-Lv used in the present reaction may be 1 equivalent or more with respect to the compound represented by Formula (1-k), is not particularly limited as long as the intended reaction proceeds, and is preferably 1 equivalent or more and 10 equivalents or less.

Examples of the base used in the present reaction include inorganic bases such as sodium hydroxide, potassium hydroxide, sodium carbonate, potassium carbonate, cesium carbonate, and sodium hydride, organic bases such as triethylamine, tributylamine, diisopropylethylamine, 1,8-diazabicyclo[5.4.0]-7-undecene, and 1,4-diazabicyclo[2.2.2]octane, metal hydrides such as sodium hydride, organic lithiums such as methyllithium, butyllithium, sec-butyllithium, t-butyllithium, and hexyllithium, and metal amides such as lithium diisopropylamide, hexamethyldisilazane lithium, hexamethyldisilazane sodium, and hexamethyldisilazane potassium.

The amount of base used in the present reaction may be 1 equivalent or more with respect to the compound represented by Formula (1-k), is not particularly limited as long as the intended reaction proceeds, and is preferably 1 equivalent or more and 10 equivalents or less.

The solvent used in the present reaction is not particularly limited as long as the intended reaction proceeds, and examples thereof include ether-based solvents such as diethyl ether, diisopropyl ether, methyl-t-butyl ether, dimethoxyethane, tetrahydrofuran, and dioxane, benzene-based solvents such as benzene, toluene, xylene, mesitylene, chlorobenzene, and dichlorobenzene, ester-based solvents such as ethyl acetate, isopropyl acetate, and butyl acetate, nitrile-based solvents such as acetonitrile, amide-based solvents such as N-methylpyrrolidone, N,N-dimethylformamide, and N,N-dimethylacetamide, urea-based solvents such as 1,3-dimethyl-2 imidazolidinone, and halogen-based solvents such as dichloromethane, dichloroethane, chloroform, and carbon tetrachloride. These solvents can be used alone or as a mixture of two or more kinds thereof in an arbitrary ratio.

The amount of solvent used in the present reaction is not particularly limited as long as the desired reaction proceeds, and is usually 3 to 200 times by weight with respect to the compound represented by Formula (1-k).

The temperature for performing the present reaction is not particularly limited as long as the intended reaction proceeds, but is usually −10° C. or higher and 180° C. or lower or below a boiling point of the solvent.

As the post-treatment of the reaction, a liquid separation operation can be performed by adding water or an appropriate aqueous solution to the reaction mixture. When an aqueous solution is used, an acidic aqueous solution in which hydrochloric acid, sulfuric acid, ammonium chloride, or the like is dissolved, an alkaline aqueous solution in which potassium hydroxide, sodium hydroxide, sodium carbonate, potassium carbonate, sodium hydrogen carbonate, potassium hydrogen carbonate, or the like is dissolved, an aqueous solution containing salts containing sulfur atoms such as sodium thiosulfate and sodium sulfite, a saline solution, or the like can be arbitrarily used. In the liquid separation operation, it is possible to add a solvent that is not compatible with water, such as a benzene-based solvent such as toluene, xylene, benzene, chlorobenzene, or dichlorobenzene, an ester-based solvent such as ethyl acetate, isopropyl acetate, or butyl acetate, an ether-based solvent such as diethyl ether, diisopropyl ether, or methyl-t-butyl ether, a halogen-based solvent such as dichloromethane, dichloroethane, or chloroform, or a hydrocarbon-based solvent such as hexane, heptane, cyclohexane, or methylcyclohexane, as necessary. In addition, these solvents can be used alone, or two or more kinds thereof can be mixed at an arbitrary ratio. The number of times of liquid separation is not particularly limited, and the liquid separation can be performed according to the intended purity and yield.

In the reaction mixture containing the compound represented by Formula (1-1) obtained above, moisture can be removed with a desiccant such as sodium sulfate or magnesium sulfate, but it is not essential.

The reaction mixture containing the compound represented by Formula (1-1) obtained above can be subjected to solvent distillation under reduced pressure as long as the compounds are not decomposed.

A reaction mixture containing the compound represented by Formula (1-1) obtained after solvent distillation can be purified by washing, reprecipitation, recrystallization, column chromatography, or the like with an appropriate solvent. It may be appropriately set according to the intended purity.

### Production Method AD

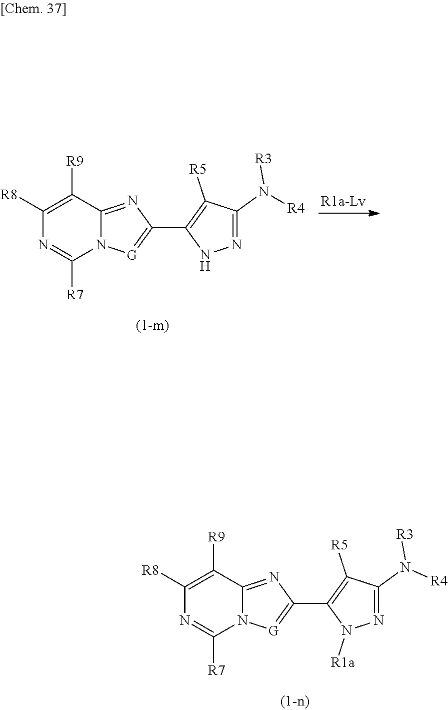

In the formula, R1a represents a C1 to C6 alkyl group optionally substituted with a substituent A, a C1 to C6 haloalkyl group, a C3 to C8 cycloalkyl group optionally substituted with a substituent B, a C2 to C6 alkenyl group optionally substituted with a substituent A, a C2 to C6 haloalkenyl group, a C2 to C6 alkynyl group optionally substituted with a substituent A, a C2 to C6 haloalkynyl group, a phenyl group optionally substituted with 0 to 5 substituents B, a 5- or 6-membered aromatic heterocyclic group optionally substituted with 0 to 4 substituents B, Rx1Rx2NC(═O)— (where Rx1 and Rx2 have the same meanings as above), Rx3C(═O)— (where Rx3 has the same meaning as above), Rx3OC(═O)— (where Rx3 has the same meaning as above), Rx4S(O)p- (where Rx4 and p have the same meanings as above), or a 3- to 6-membered ring group containing one or two oxygen atoms, and G, R3, R4, R5, R6, R7, R8, R9, and Lv have the same meanings as above.

A production method AD is a method for obtaining a compound represented by Formula (1-n), the production method comprising reacting a compound represented by Formula (1-m) with R1a-Lv in a solvent in the presence of a base.

The production method AD can be performed in accordance with the production method AC by using the compound represented by Formula (1-k) instead of the compound represented by Formula (1-m) and using R2a-Lv instead of R1a-Lv in the production method AC.

### Production Method AE

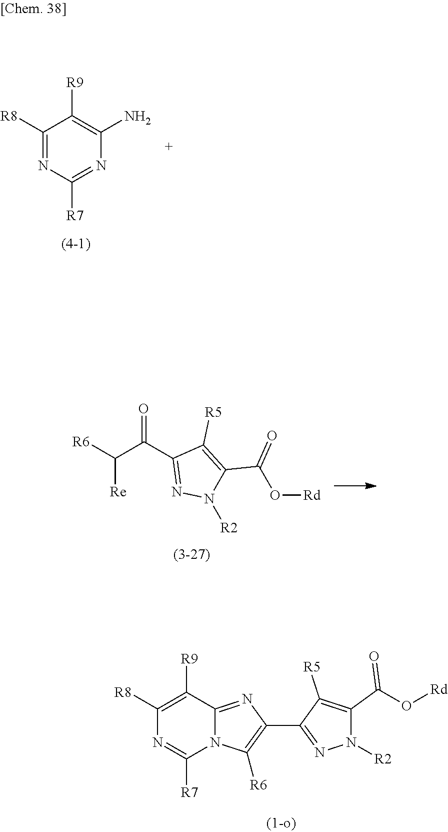

In the formula, R2, R5, R6, R7, R8, R9, Rd, and Re have the same meanings as above.

A production method AE is a method for obtaining a compound represented by Formula (1-o), the production method comprising reacting a compound represented by Formula (4-1) with a compound represented by Formula (3-27) in a solvent.

A base can be used in the present reaction. Examples of the base used include inorganic bases such as sodium hydroxide, potassium hydroxide, sodium carbonate, potassium carbonate, cesium carbonate, and sodium hydride, organic bases such as triethylamine, tributylamine, diisopropylethylamine, 1,8-diazabicyclo[5.4.0]-7-undecene, and 1,4-diazabicyclo[2.2.2]octane, metal hydrides such as sodium hydride, organic lithiums such as methyllithium, butyllithium, sec-butyllithium, t-butyllithium, and hexyllithium, and metal amides such as lithium diisopropylamide, hexamethyldisilazane lithium, hexamethyldisilazane sodium, and hexamethyldisilazane potassium.

The amount of base used in the present reaction may be 1 equivalent or more with respect to the compound represented by Formula (3-27), is not particularly limited as long as the intended reaction proceeds, and is preferably 1 equivalent or more and 10 equivalents or less.

The solvent used in the present reaction is not particularly limited as long as the intended reaction proceeds, and examples thereof include ether-based solvents such as diethyl ether, diisopropyl ether, methyl-t-butyl ether, dimethoxyethane, tetrahydrofuran, and dioxane, benzene-based solvents such as benzene, toluene, xylene, mesitylene, chlorobenzene, and dichlorobenzene, ester-based solvents such as ethyl acetate, isopropyl acetate, and butyl acetate, nitrile-based solvents such as acetonitrile, amide-based solvents such as N-methylpyrrolidone, N,N-dimethylformamide, and N,N-dimethylacetamide, urea-based solvents such as 1,3-dimethyl-2 imidazolidinone, and halogen-based solvents such as dichloromethane, dichloroethane, chloroform, and carbon tetrachloride. These solvents can be used alone or as a mixture of two or more kinds thereof in an arbitrary ratio.

The amount of solvent used in the present reaction is not particularly limited as long as the desired reaction proceeds, and is usually 3 to 200 times by weight with respect to the compound represented by Formula (3-27).

The temperature for performing the present reaction is not particularly limited as long as the intended reaction proceeds, but is usually −10° C. or higher and 180° C. or lower or below a boiling point of the solvent.

As the post-treatment of the reaction, a liquid separation operation can be performed by adding water or an appropriate aqueous solution to the reaction mixture. When an aqueous solution is used, an acidic aqueous solution in which hydrochloric acid, sulfuric acid, ammonium chloride, or the like is dissolved, an alkaline aqueous solution in which potassium hydroxide, sodium hydroxide, sodium carbonate, potassium carbonate, sodium hydrogen carbonate, potassium hydrogen carbonate, or the like is dissolved, an aqueous solution containing salts containing sulfur atoms such as sodium thiosulfate and sodium sulfite, a saline solution, or the like can be arbitrarily used. In the liquid separation operation, it is possible to add a solvent that is not compatible with water, such as a benzene-based solvent such as toluene, xylene, benzene, chlorobenzene, or dichlorobenzene, an ester-based solvent such as ethyl acetate, isopropyl acetate, or butyl acetate, an ether-based solvent such as diethyl ether, diisopropyl ether, or methyl-t-butyl ether, a halogen-based solvent such as dichloromethane, dichloroethane, or chloroform, or a hydrocarbon-based solvent such as hexane, heptane, cyclohexane, or methylcyclohexane, as necessary. In addition, these solvents can be used alone, or two or more kinds thereof can be mixed at an arbitrary ratio. The number of times of liquid separation is not particularly limited, and the liquid separation can be performed according to the intended purity and yield.

In the reaction mixture containing the compound represented by Formula (1-o) obtained above, moisture can be removed with a desiccant such as sodium sulfate or magnesium sulfate, but it is not essential.

The reaction mixture containing the compound represented by Formula (1-o) obtained above can be subjected to solvent distillation under reduced pressure as long as the compounds are not decomposed.

A reaction mixture containing the compound represented by Formula (1-o) obtained after solvent distillation can be purified by washing, reprecipitation, recrystallization, column chromatography, or the like with an appropriate solvent. It may be appropriately set according to the intended purity.

### Production Method AF

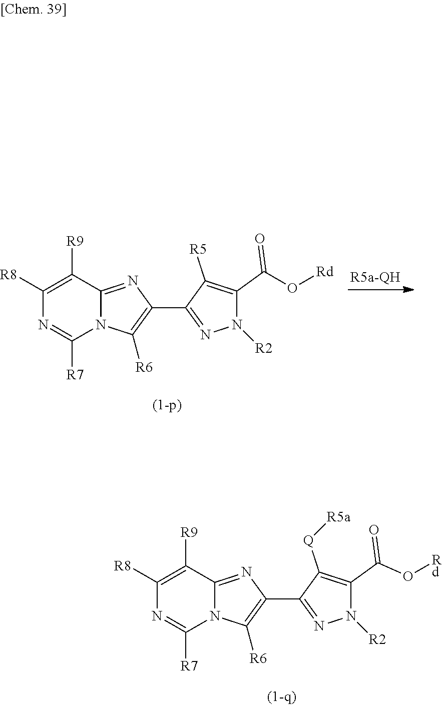

In the formula, R2, R5a, R6, R7, R8, R9, Q, Rc, and Rd have the same meanings as above.

A production method AF is a method for synthesizing a compound represented by Formula (1-q), the production method comprising reacting a compound represented by Formula (1-p) with R5a-QH (where R5a has the same meaning as above) in a solvent in the presence of a base.

The production method AF can be performed in accordance with the production method P by using the compound represented by Formula (3-18) instead of the compound represented by Formula (1-p) in the production method P.

### Production Method AG

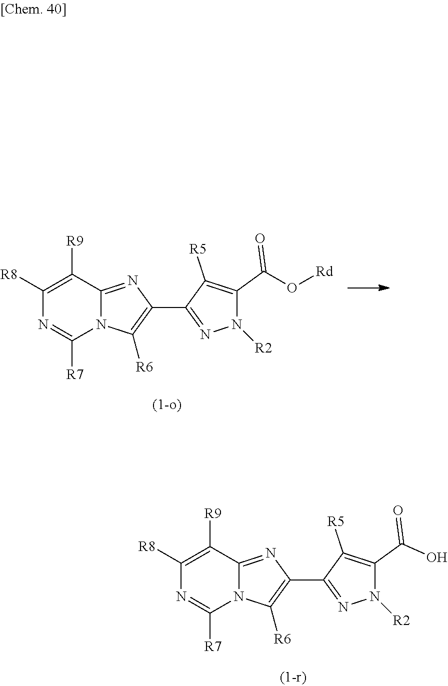

In the formula, R2, R5, R6, R7, R8, R9, and Rd have the same meanings as above.

A production method AG is a method for synthesizing a compound represented by Formula (1-r), which is a production intermediate of the compound of the present invention, the production method comprising reacting a compound represented by Formula (1-o) in a solvent under acidic or basic conditions.

First, the reaction under acidic conditions will be described.

Examples of the acid used in the present reaction include inorganic acids such as hydrochloric acid, hydrobromic acid, and phosphoric acid, and organic acids such as acetic acid, methanesulfonic acid, p-toluenesulfonic acid, and trifluoroacetic acid. There is no particular limitation as long as the intended reaction proceeds.

The amount of acid used in the present reaction may be a catalytic amount, is not particularly limited as long as the intended reaction proceeds, and is preferably 0.01 equivalents or more with respect to the compound represented by Formula (1-o). In addition, a liquid acid can also be used as a solvent.

The solvent used in the present reaction is not particularly limited as long as the intended reaction proceeds, and examples thereof include an aqueous solvent, acidic solvents such as acetic acid and methanesulfonic acid, ether-based solvents such as diethyl ether, diisopropyl ether, methyl-t-butyl ether, dimethoxyethane, tetrahydrofuran, and dioxane, alcohol-based solvents such as methanol, ethanol, and isopropanol, benzene-based solvents such as benzene, toluene, xylene, mesitylene, chlorobenzene, and dichlorobenzene, ester-based solvents such as ethyl acetate, isopropyl acetate, and butyl acetate, nitrile-based solvents such as acetonitrile, amide-based solvents such as N-methylpyrrolidone, N,N-dimethylformamide, and N,N-dimethylacetamide, urea-based solvents such as 1,3-dimethyl-2 imidazolidinone, and halogen-based solvents such as dichloromethane, dichloroethane, chloroform, and carbon tetrachloride. These solvents can be used alone or as a mixture of two or more kinds thereof in an arbitrary ratio.

The amount of solvent used in the present reaction is not particularly limited as long as the desired reaction proceeds, and is usually 3 to 200 times by weight with respect to the compound represented by Formula (1-0).

The temperature for performing the present reaction is not particularly limited as long as the intended reaction proceeds, but is usually 0° C. or higher and 180° C. or lower or below a boiling point of the solvent.

Next, the reaction under basic conditions will be described.

Examples of the base used in the present reaction include inorganic bases such as lithium hydroxide, sodium hydroxide, and potassium hydroxide, and are not particularly limited as long as the intended reaction proceeds.

The base used in the present reaction may be 1 equivalent or more with respect to the compound represented by Formula (1-o), is not particularly limited as long as the intended reaction proceeds, and is preferably 1 equivalent or more and 30 equivalents or less.

The solvent used in the present reaction is not particularly limited as long as the intended reaction proceeds, and examples thereof include an aqueous solvent, ether-based solvents such as diethyl ether, diisopropyl ether, methyl-t-butyl ether, dimethoxyethane, tetrahydrofuran, and dioxane, alcohol-based solvents such as methanol, ethanol, and isopropanol, benzene-based solvents such as benzene, toluene, xylene, mesitylene, chlorobenzene, and dichlorobenzene, ester-based solvents such as ethyl acetate, isopropyl acetate, and butyl acetate, nitrile-based solvents such as acetonitrile, amide-based solvents such as N-methylpyrrolidone, N,N-dimethylformamide, and N,N-dimethylacetamide, urea-based solvents such as 1,3-dimethyl-2 imidazolidinone, and halogen-based solvents such as dichloromethane, dichloroethane, chloroform, and carbon tetrachloride. These solvents can be used alone or as a mixture of two or more kinds thereof in an arbitrary ratio.

The amount of solvent used in the present reaction is not particularly limited as long as the desired reaction proceeds, and is usually 3 to 200 times by weight with respect to the compound represented by Formula (1-o).

The temperature for performing the present reaction is not particularly limited as long as the intended reaction proceeds, but is usually −20° C. or higher and 180° C. or lower or below a boiling point of the solvent.

In the post-treatment of the reaction, the reaction under acidic conditions and the reaction under basic conditions can be performed by a common method. A liquid separation operation can be performed by adding water or an appropriate aqueous solution to the reaction mixture. When an aqueous solution is used, an acidic aqueous solution in which hydrochloric acid, sulfuric acid, or the like is dissolved, an alkaline aqueous solution in which potassium hydroxide, sodium hydroxide, sodium carbonate, potassium carbonate, sodium hydrogen carbonate, potassium hydrogen carbonate, or the like is dissolved, a saline solution, or the like can be arbitrarily used. In the liquid separation operation, it is possible to add a solvent that is not compatible with water, such as a benzene-based solvent such as toluene, xylene, benzene, chlorobenzene, or dichlorobenzene, an ester-based solvent such as ethyl acetate, isopropyl acetate, or butyl acetate, an ether-based solvent such as diethyl ether, diisopropyl ether, or methyl-t-butyl ether, a halogen-based solvent such as dichloromethane, dichloroethane, chloroform, or carbon tetrachloride, or a hydrocarbon-based solvent such as hexane, heptane, cyclohexane, or methylcyclohexane, as necessary. In addition, these solvents can be used alone, or two or more kinds thereof can be mixed at an arbitrary ratio. The number of times of liquid separation is not particularly limited, and the liquid separation can be performed according to the intended purity and yield.

In the reaction mixture containing the compound represented by Formula (1-r) obtained above, moisture can be removed with a desiccant such as sodium sulfate or magnesium sulfate, but it is not essential.

The reaction mixture containing the compound represented by Formula (1-r) obtained above can be subjected to solvent distillation under reduced pressure as long as the compounds are not decomposed.

A reaction mixture containing the compound represented by Formula (1-r) obtained after solvent distillation can be purified by washing, reprecipitation, recrystallization, column chromatography, or the like with an appropriate solvent. It may be appropriately set according to the intended purity.

The compound represented by Formula (1-r) can be a useful intermediate for obtaining the compound represented by Formula (1-2) which is the compound of the present invention. A compound represented by Formula (1-2) can be obtained by subjecting the compound represented by Formula (1-r) to a Curtius rearrangement reaction.

Specific examples of the production intermediate represented by Formula (1-r) are represented by a combination of Structural Formulas E-1 to E-24 (where R2 and p have the same meanings as above) and HetA (where HetA represents a structural formula of any one of C-1 to C-224 in Table 1) shown in Table 5. These compounds are for illustrative purposes and the present invention is not limited thereto.

TABLE 5

| | |
|---|---|
| 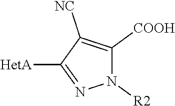 | E-1 |
| 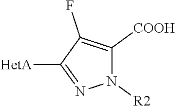 | E-2 |
| 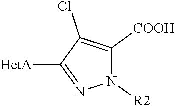 | E-3 |
| 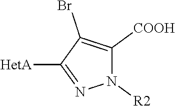 | E-4 |
| 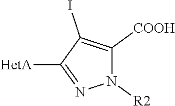 | E-5 |
| 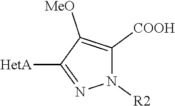 | E-6 |
| 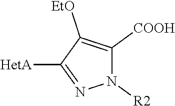 | E-7 |
| 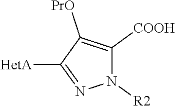 | E-8 |
| 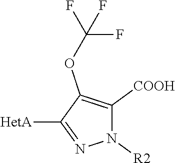 | E-9 |
| 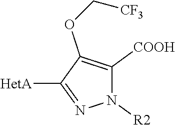 | E-10 |
| 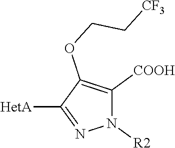 | E-11 |
| 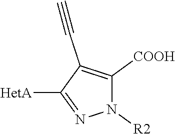 | E-12 |
| 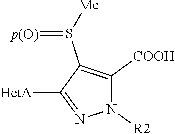 | E-13 |
| 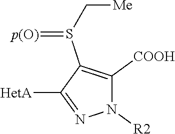 | E-14 |
| 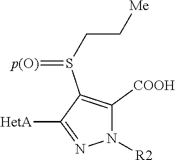 | E-15 |
| 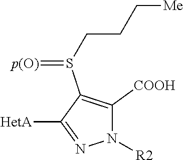 | E-16 |
| 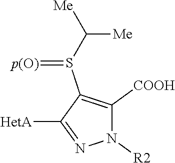 | E-17 |
| 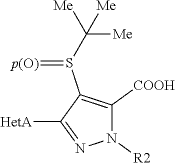 | E-18 |
| 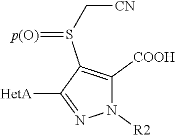 | E-19 |
| 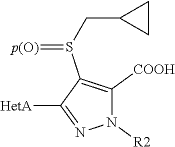 | E-20 |
| 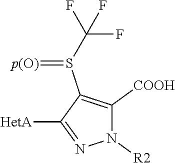 | E-21 |
| 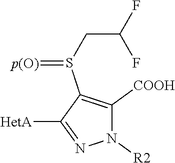 | E-22 |
| 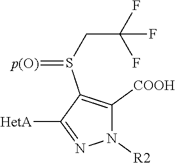 | E-23 |
| 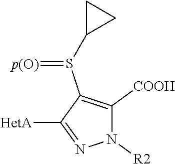 | E-24 |

Various reaction conditions are known for the Curtius rearrangement reaction (Non Patent Literature: L. Kuirti, B. Czakó, “Strategic Applications of Named Reactions in Organic Synthesis” Elsevier, Amsterdam, 2005). Conditions for obtaining the compound represented by Formula (1) of the present invention may be appropriately set. Here, an example in which t-butanol is used in the production method AH will be described.

### Production Method AH

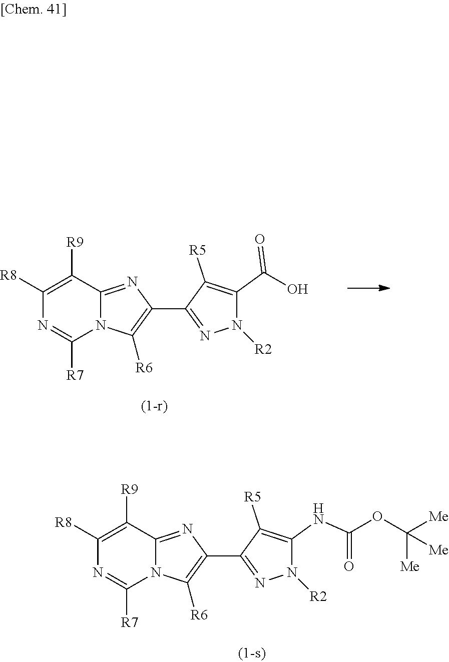

In the formula, R2, R5, R6, R7, R8, and R9 have the same meanings as above.

A production method AH is a method for obtaining a compound represented by Formula (1-s), the production method comprising reacting the compound represented by Formula (1-r) with an azide compound in a solvent in the presence of t-butanol.

Examples of the azide compound used in the present reaction include diphenyl phosphate azide.

The amount of azide compound used in the present reaction may be 1 equivalent or more with respect to the compound represented by Formula (1-r), is not particularly limited as long as the intended reaction proceeds, and is usually 1 equivalent or more and 10 equivalents or less.

The amount of t-butanol used in the present reaction may be 1 equivalent or more with respect to the compound represented by Formula (1-r), and is not particularly limited as long as the intended reaction proceeds. t-butanol itself can also be used as a solvent.

Examples of the solvent used in the present reaction include alcohol-based solvents such as t-butanol, ether-based solvents such as diisopropyl ether, methyl-t-butyl ether, dimethoxyethane, tetrahydrofuran, and dioxane, and benzene-based solvents such as toluene, xylene, benzene, chlorobenzene, and dichlorobenzene. These solvents can be used alone or as a mixture of two or more kinds thereof in an arbitrary ratio.

The amount of solvent used in the present reaction is not particularly limited as long as the desired reaction proceeds, and is usually 3 to 200 times by weight with respect to the compound represented by Formula (1-r).

The temperature for performing the present reaction is not particularly limited as long as the intended reaction proceeds, but is usually 0° C. or higher and 150° C. or lower or below a boiling point of the solvent.

As the post-treatment of the reaction, a liquid separation operation can be performed by adding water or an appropriate aqueous solution to the reaction mixture. When an aqueous solution is used, an acidic aqueous solution in which hydrochloric acid, sulfuric acid, ammonium chloride, or the like is dissolved, an alkaline aqueous solution in which potassium hydroxide, sodium hydroxide, sodium carbonate, potassium carbonate, sodium hydrogen carbonate, potassium hydrogen carbonate, or the like is dissolved, an aqueous solution containing salts containing sulfur atoms such as sodium thiosulfate and sodium sulfite, a saline solution, or the like can be arbitrarily used. In the liquid separation operation, it is possible to add a solvent that is not compatible with water, such as a benzene-based solvent such as toluene, xylene, benzene, chlorobenzene, or dichlorobenzene, an ester-based solvent such as ethyl acetate, isopropyl acetate, or butyl acetate, an ether-based solvent such as diethyl ether, diisopropyl ether, or methyl-t-butyl ether, a halogen-based solvent such as dichloromethane, dichloroethane, or chloroform, or a hydrocarbon-based solvent such as hexane, heptane, cyclohexane, or methylcyclohexane, as necessary. In addition, these solvents can be used alone, or two or more kinds thereof can be mixed at an arbitrary ratio. The number of times of liquid separation is not particularly limited, and the liquid separation can be performed according to the intended purity and yield.

In the reaction mixture containing the compound represented by Formula (1-s) obtained above, moisture can be removed with a desiccant such as sodium sulfate or magnesium sulfate, but it is not essential.

The reaction mixture containing the compound represented by Formula (1-s) obtained above can be subjected to solvent distillation under reduced pressure as long as the compounds are not decomposed.

A reaction mixture containing the compound represented by Formula (1-s) obtained after solvent distillation can be purified by washing, reprecipitation, recrystallization, column chromatography, or the like with an appropriate solvent. It may be appropriately set according to the intended purity.

### Production Method AI

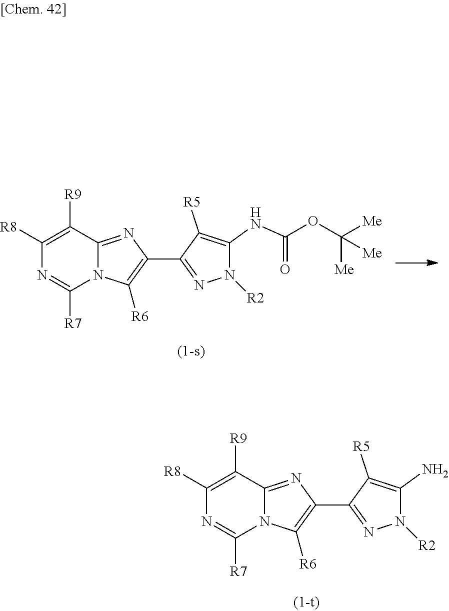

In the formula, R2, R5, R6, R7, R8, and R9 have the same meanings as above.

A production method AI is a method for obtaining a compound represented by Formula (1-t), the production method comprising reacting a compound represented by Formula (1-s) with an acid in a solvent.

Examples of the acid used in the present reaction include inorganic acids such as hydrochloric acid and hydrobromic acid, and organic acids such as methanesulfonic acid, p-toluenesulfonic acid, and trifluoroacetic acid. There is no particular limitation as long as the intended reaction proceeds.

The amount of acid used in the present reaction may be a catalytic amount with respect to the compound represented by Formula (1-s), is not particularly limited as long as the intended reaction proceeds, is usually 1 equivalent or more, and can be used as a solvent.

The solvent used in the present reaction is not particularly limited as long as the intended reaction proceeds, and examples thereof include acid solvents such as hydrochloric acid and trifluoroacetic acid, ether-based solvents such as diethyl ether, diisopropyl ether, methyl-t-butyl ether, dimethoxyethane, tetrahydrofuran, and dioxane, alcohol-based solvents such as methanol, ethanol, and isopropanol, benzene-based solvents such as benzene, toluene, xylene, mesitylene, chlorobenzene, and dichlorobenzene, ester-based solvents such as ethyl acetate, isopropyl acetate, and butyl acetate, and halogen-based solvents such as dichloromethane, dichloroethane, chloroform, and carbon tetrachloride. These solvents can be used alone or as a mixture of two or more kinds thereof in an arbitrary ratio.

The amount of solvent used in the present reaction is not particularly limited as long as the desired reaction proceeds, and is usually 3 to 200 times by weight with respect to the compound represented by Formula (1-s).

The temperature for performing the present reaction is not particularly limited as long as the intended reaction proceeds, but is usually −20° C. or higher and 180° C. or lower or below a boiling point of the solvent.

As the post-treatment of the reaction, a liquid separation operation can be performed by adding water or an appropriate aqueous solution to the reaction mixture. When an aqueous solution is used, an acidic aqueous solution in which hydrochloric acid, sulfuric acid, ammonium chloride, or the like is dissolved, an alkaline aqueous solution in which potassium hydroxide, sodium hydroxide, sodium carbonate, potassium carbonate, sodium hydrogen carbonate, potassium hydrogen carbonate, or the like is dissolved, an aqueous solution containing salts containing sulfur atoms such as sodium thiosulfate and sodium sulfite, a saline solution, or the like can be arbitrarily used. In the liquid separation operation, it is possible to add a solvent that is not compatible with water, such as a benzene-based solvent such as toluene, xylene, benzene, chlorobenzene, or dichlorobenzene, an ester-based solvent such as ethyl acetate, isopropyl acetate, or butyl acetate, an ether-based solvent such as diethyl ether, diisopropyl ether, or methyl-t-butyl ether, a halogen-based solvent such as dichloromethane, dichloroethane, chloroform, or carbon tetrachloride, or a hydrocarbon-based solvent such as hexane, heptane, cyclohexane, or methylcyclohexane, as necessary. In addition, these solvents can be used alone, or two or more kinds thereof can be mixed at an arbitrary ratio. The number of times of liquid separation is not particularly limited, and the liquid separation can be performed according to the intended purity and yield.

In the reaction mixture containing the compound represented by Formula (1-t) obtained above, moisture can be removed with a desiccant such as sodium sulfate or magnesium sulfate, but it is not essential.

The reaction mixture containing the compound represented by Formula (1-t) obtained above can be subjected to solvent distillation under reduced pressure as long as the compounds are not decomposed.

A reaction mixture containing the compound represented by Formula (1-t) obtained after solvent distillation can be purified by washing, reprecipitation, recrystallization, column chromatography, or the like with an appropriate solvent. It may be appropriately set according to the intended purity.

### Production Method AJ

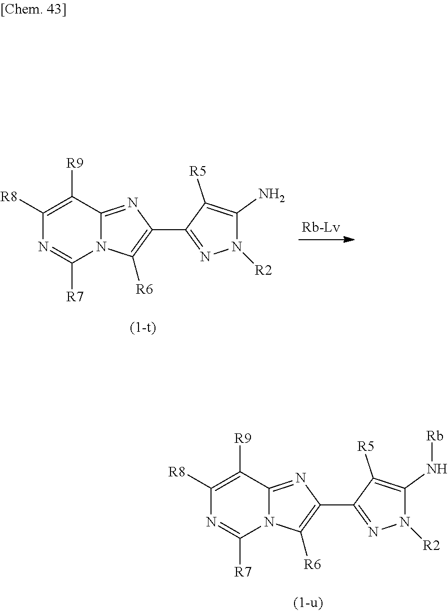

In the formula, R2, R5, R6, R7, R8, R9, Rb and Lv have the same meanings as above.

A production method AJ is a method for obtaining a compound represented by Formula (1-u), the production method comprising reacting a compound represented by Formula (1-t) with a compound represented by Rb-Lv in a solvent.

The production method AJ can be performed in accordance with the production method B by using the compound represented by Formula (3-2) instead of the compound represented by Formula (1-t) in the production method B.

### Production Method AK

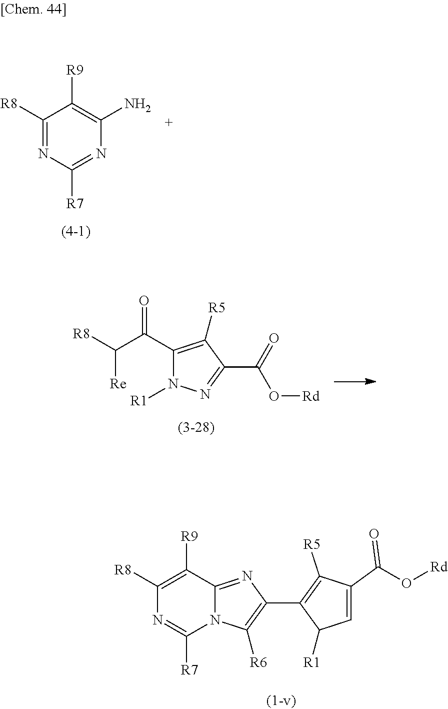

In the formula, R1, R5, R6, R7, R8, R9, Rd, and Re have the same meanings as above.

A production method AK is a method for obtaining a compound represented by Formula (1-v), the production method comprising reacting a compound represented by Formula (4-1) with a compound represented by Formula (3-28) in a solvent.

The production method AK can be performed in accordance with the production method AE by using the compound represented by Formula (3-27) instead of the compound represented by Formula (3-28) in the production method AE.

### Production Method AL

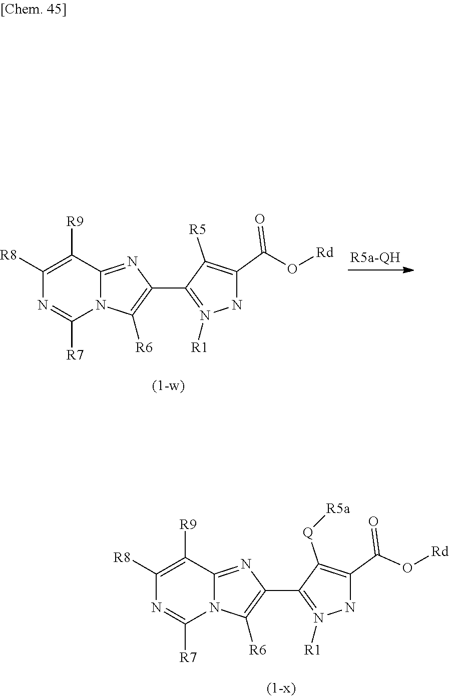

In the formula, R1, R5a, R6, R7, R8, R9, Rc, Rd, and Q have the same meanings as above.

A production method AL is a method for synthesizing a compound represented by Formula (1-x), the production method comprising reacting a compound represented by Formula (1-w) with R5a-QH (where R5a has the same meaning as above) in a solvent in the presence of a base.

The production method AF can be performed in accordance with the production method T by using the compound represented by Formula (3-23) instead of the compound represented by Formula (1-w) in the production method T.

### Production Method AM

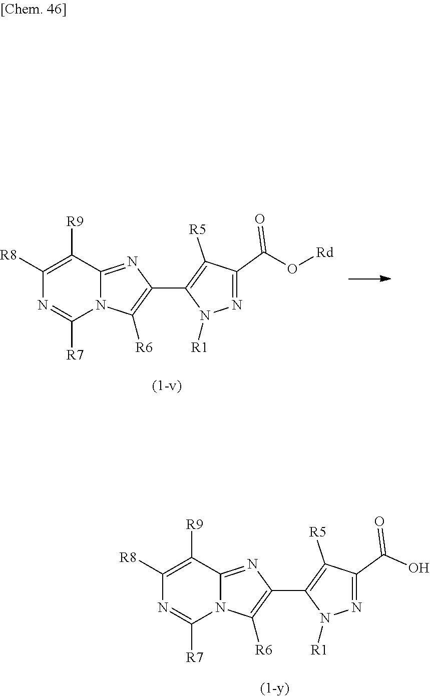

In the formula, R1, R5, R6, R7, R8, R9, and Rd have the same meanings as above.

A production method AM is a method for synthesizing a compound represented by Formula (1-y), the production method comprising reacting a compound represented by Formula (1-v) in a solvent under acidic or basic conditions.

The production method AM can be performed in accordance with the production method AG by using the compound represented by Formula (1-o) instead of the compound represented by Formula (1-v) in the production method AG.

The compound represented by Formula (1-y) can be a useful intermediate for obtaining the compound represented by Formula (1-1) which is the compound of the present invention. A compound represented by Formula (1-1) can be obtained by subjecting the compound represented by Formula (1-y) to a Curtius rearrangement reaction.

Specific examples of the production intermediate represented by Formula (1-y) are represented by a combination of Structural Formulas D-1 to D-24 (where R1 and p have the same meanings as above) and HetA (where HetA represents a structural formula of any one of C-1 to C-224 in Table 1) shown in Table 6. These compounds are for illustrative purposes and the present invention is not limited thereto.

TABLE 6
Table 6
D-1 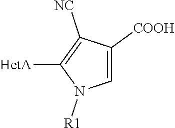
D-2 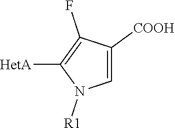
D-3 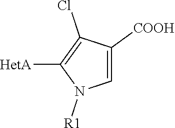
D-4 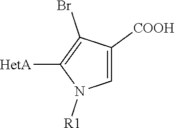
D-5 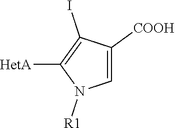
D-6 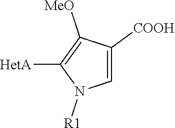
D-7 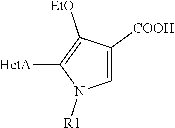
D-8 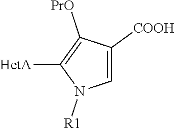
D-9 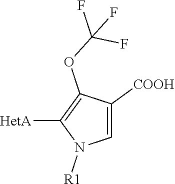
D-10 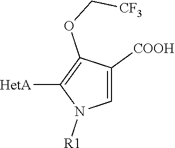
D-11 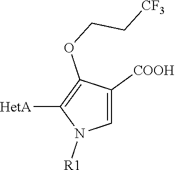
D-12 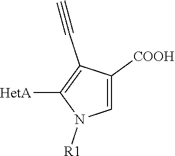
D-13 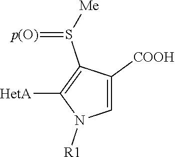
D-14 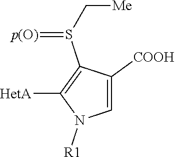
D-15 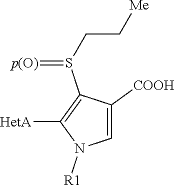
D-16 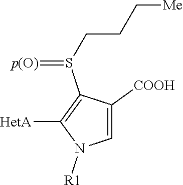
D-17 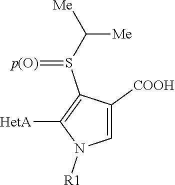
D-18 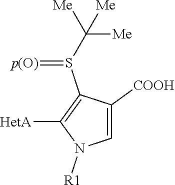
D-19 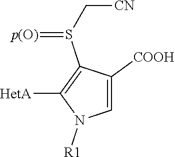
D-20 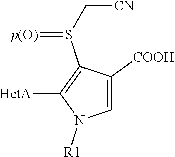
D-21 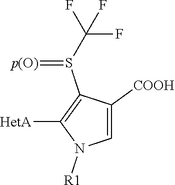
D-22 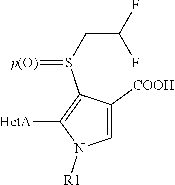
D-23 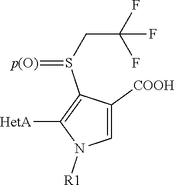
D-24

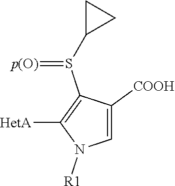

Various reaction conditions are known for the Curtius rearrangement reaction (Non Patent Literature: L. Küirti, B. Czak6, “Strategic Applications of Named Reactions in Organic Synthesis” Elsevier, Amsterdam, 2005). Conditions for obtaining the compound represented by Formula (1-1) of the present invention may be appropriately set. Here, an example in which t-butanol is used in the production method AN will be described.

### Production Method AN

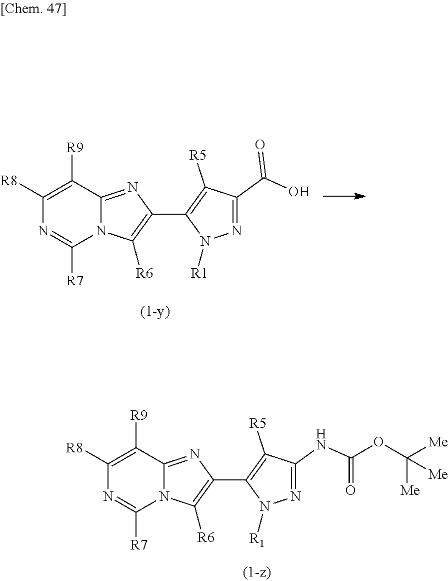

In the formula, R1, R5, R6, R7, R8, and R9 have the same meanings as above.

A production method AN is a method for obtaining a compound represented by Formula (1-z), the production method comprising reacting the compound represented by Formula (1-y) with an azide compound in a solvent in the presence of t-butanol.

Examples of the azide compound used in the present reaction include diphenyl phosphate azide.

The amount of azide compound used in the present reaction may be 1 equivalent or more with respect to the compound represented by Formula (1-y), is not particularly limited as long as the intended reaction proceeds, and is usually 1 equivalent or more and 10 equivalents or less.

The amount of t-butanol used in the present reaction may be 1 equivalent or more with respect to the compound represented by Formula (1-y), and is not particularly limited as long as the intended reaction proceeds. Tertiary butanol itself can also be used as a solvent.

Examples of the solvent used in the present reaction include alcohol-based solvents such as t-butanol, ether-based solvents such as diisopropyl ether, methyl-t-butyl ether, dimethoxyethane, tetrahydrofuran, and dioxane, and benzene-based solvents such as toluene, xylene, benzene, chlorobenzene, and dichlorobenzene. These solvents can be used alone or as a mixture of two or more kinds thereof in an arbitrary ratio.

The amount of solvent used in the present reaction is not particularly limited as long as the desired reaction proceeds, and is usually 3 to 200 times by weight with respect to the compound represented by Formula (1-y).

The temperature for performing the present reaction is not particularly limited as long as the intended reaction proceeds, but is usually 0° C. or higher and 150° C. or lower or below a boiling point of the solvent.

As the post-treatment of the reaction, a liquid separation operation can be performed by adding water or an appropriate aqueous solution to the reaction mixture. When an aqueous solution is used, an acidic aqueous solution in which hydrochloric acid, sulfuric acid, ammonium chloride, or the like is dissolved, an alkaline aqueous solution in which potassium hydroxide, sodium hydroxide, sodium carbonate, potassium carbonate, sodium hydrogen carbonate, potassium hydrogen carbonate, or the like is dissolved, an aqueous solution containing salts containing sulfur atoms such as sodium thiosulfate and sodium sulfite, a saline solution, or the like can be arbitrarily used. In the liquid separation operation, it is possible to add a solvent that is not compatible with water, such as a benzene-based solvent such as toluene, xylene, benzene, chlorobenzene, or dichlorobenzene, an ester-based solvent such as ethyl acetate, isopropyl acetate, or butyl acetate, an ether-based solvent such as diethyl ether, diisopropyl ether, or methyl-t-butyl ether, a halogen-based solvent such as dichloromethane, dichloroethane, or chloroform, or a hydrocarbon-based solvent such as hexane, heptane, cyclohexane, or methylcyclohexane, as necessary. In addition, these solvents can be used alone, or two or more kinds thereof can be mixed at an arbitrary ratio. The number of times of liquid separation is not particularly limited, and the liquid separation can be performed according to the intended purity and yield.

In the reaction mixture containing the compound represented by Formula (1-z) obtained above, moisture can be removed with a desiccant such as sodium sulfate or magnesium sulfate, but it is not essential.

The reaction mixture containing the compound represented by Formula (1-z) obtained above can be subjected to solvent distillation under reduced pressure as long as the compounds are not decomposed.

A reaction mixture containing the compound represented by Formula (1-z) obtained after solvent distillation can be purified by washing, reprecipitation, recrystallization, column chromatography, or the like with an appropriate solvent. It may be appropriately set according to the intended purity.

### Production Method AO

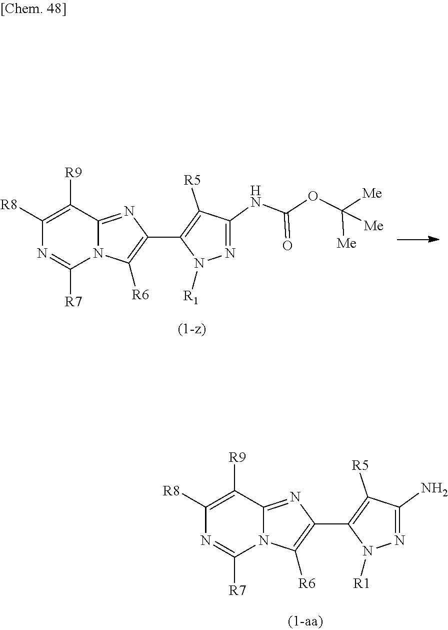

In the formula, R1, R5, R6, R7, R8, and R9 have the same meanings as above.

A production method AO is a method for obtaining a compound represented by Formula (1-aa), the production method comprising reacting a compound represented by Formula (1-z) with an acid in a solvent.

Examples of the acid used in the present reaction include inorganic acids such as hydrochloric acid and hydrobromic acid, and organic acids such as methanesulfonic acid, p-toluenesulfonic acid, and trifluoroacetic acid. There is no particular limitation as long as the intended reaction proceeds.

The amount of acid used in the present reaction may be a catalytic amount with respect to the compound represented by Formula (1-z), is not particularly limited as long as the intended reaction proceeds, is usually 1 equivalent or more, and can be used as a solvent.

The solvent used in the present reaction is not particularly limited as long as the intended reaction proceeds, and examples thereof include acid solvents such as hydrochloric acid and trifluoroacetic acid, ether-based solvents such as diethyl ether, diisopropyl ether, methyl-t-butyl ether, dimethoxyethane, tetrahydrofuran, and dioxane, alcohol-based solvents such as methanol, ethanol, and isopropanol, benzene-based solvents such as benzene, toluene, xylene, mesitylene, chlorobenzene, and dichlorobenzene, ester-based solvents such as ethyl acetate, isopropyl acetate, and butyl acetate, and halogen-based solvents such as dichloromethane, dichloroethane, chloroform, and carbon tetrachloride. These solvents can be used alone or as a mixture of two or more kinds thereof in an arbitrary ratio.

The amount of solvent used in the present reaction is not particularly limited as long as the desired reaction proceeds, and is usually 3 to 200 times by weight with respect to the compound represented by Formula (1-z).

The temperature for performing the present reaction is not particularly limited as long as the intended reaction proceeds, but is usually −20° C. or higher and 180° C. or lower or below a boiling point of the solvent.

As the post-treatment of the reaction, a liquid separation operation can be performed by adding water or an appropriate aqueous solution to the reaction mixture. When an aqueous solution is used, an acidic aqueous solution in which hydrochloric acid, sulfuric acid, ammonium chloride, or the like is dissolved, an alkaline aqueous solution in which potassium hydroxide, sodium hydroxide, sodium carbonate, potassium carbonate, sodium hydrogen carbonate, potassium hydrogen carbonate, or the like is dissolved, an aqueous solution containing salts containing sulfur atoms such as sodium thiosulfate and sodium sulfite, a saline solution, or the like can be arbitrarily used. In the liquid separation operation, it is possible to add a solvent that is not compatible with water, such as a benzene-based solvent such as toluene, xylene, benzene, chlorobenzene, or dichlorobenzene, an ester-based solvent such as ethyl acetate, isopropyl acetate, or butyl acetate, an ether-based solvent such as diethyl ether, diisopropyl ether, or methyl-t-butyl ether, a halogen-based solvent such as dichloromethane, dichloroethane, chloroform, or carbon tetrachloride, or a hydrocarbon-based solvent such as hexane, heptane, cyclohexane, or methylcyclohexane, as necessary. In addition, these solvents can be used alone, or two or more kinds thereof can be mixed at an arbitrary ratio. The number of times of liquid separation is not particularly limited, and the liquid separation can be performed according to the intended purity and yield.

In the reaction mixture containing the compound represented by Formula (1-aa) obtained above, moisture can be removed with a desiccant such as sodium sulfate or magnesium sulfate, but it is not essential.

The reaction mixture containing the compound represented by Formula (1-aa) obtained above can be subjected to solvent distillation under reduced pressure as long as the compounds are not decomposed.

A reaction mixture containing the compound represented by Formula (1-aa) obtained after solvent distillation can be purified by washing, reprecipitation, recrystallization, column chromatography, or the like with an appropriate solvent. It may be appropriately set according to the intended purity.

### Production Method AP

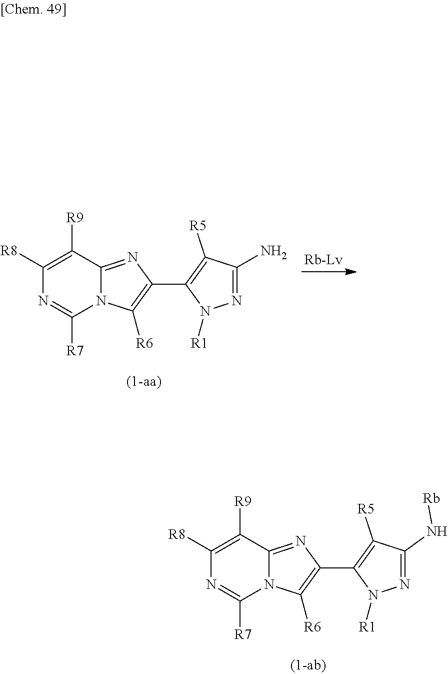

In the formula, R1, R5, R6, R7, R8, R9, Rb and Lv have the same meanings as above.

A production method AP is a method for obtaining a compound represented by Formula (1-ab), the production method comprising reacting a compound represented by Formula (1-aa) with a compound represented by Rb-Lv in a solvent.

The production method AP can be performed in accordance with the production method AJ by using the compound represented by Formula (1-t) instead of the compound represented by Formula (1-aa) in the production method AJ.

The compound represented by Formula (1-1) or Formula (1-2) can be produced by any combination of the production method A to the production method AP described above. Alternatively, the compound represented by Formula (1-1) or Formula (1-2) can be produced by any combination of the known method and the production method A to the production method AP described above.

The compound of the present invention can be used as an agricultural chemical because it can control organisms harmful to plants. Specific examples thereof include an insecticide, a bactericide, an herbicide, and a plant growth regulator. The compound of the present invention is preferably an insecticide.

Examples of the method for applying the composition containing the compound of the present invention include a method in which the composition is brought into contact with a plant body or a seed, and a method in which the composition is contained in cultivation soil and brought into contact with a root or a rhizome of a plant. Specific examples of the composition application method include a foliage spraying treatment onto individual plants, an injection treatment, a seedling box treatment, a cell tray treatment, a spraying treatment onto plant seeds, a smearing treatment to plant seeds, a dipping treatment to plant seeds, a powder coating treatment to plant seeds, a spraying treatment onto soil surface, soil mixing after a spraying treatment onto soil surface, an injection treatment into soil, soil mixing after an injection treatment in soil, a soil irrigation treatment, and soil mixing after a soil irrigation treatment. Usually, any application method available to those skilled in the art is sufficiently effective.

The “plant” referred to in the present invention refers to a thing that performs photosynthesis and lives without exercise. Specific examples thereof include rice, wheat, barley, corn, coffee, banana, grape, apple, pear, peach, cherry, persimmon, citrus, soybean, bean, cotton, strawberry, potato, cabbage, lettuce, tomato, cucumber, eggplant, watermelon, sugar beet, spinach, pea, pumpkin, sugar cane, tobacco, green pepper, sweet potato, aroid, konjac, cotton, sunflower, rose, tulip, chrysanthemum, grass, and F1 varieties thereof. In addition, genetically modified crops that are produced by artificially manipulating genes and the like and are not originally present in nature are also included, and examples thereof include agricultural and horticultural crops such as soybean, corn, cotton and the like to which herbicide resistance is imparted, rice, tobacco and the like adapted to cold regions, corn, cotton and the like to which insecticidal substance production ability is imparted. Furthermore, examples thereof include trees such as pine, ash, ginkgo, maple, oak, poplar, and zelkova. In addition, the “plant body” referred to in the present invention is a generic term for all parts constituting the plant individual, and examples thereof include a stem, a leaf, a root, a seed, a flower, and a fruit.

The term “seed” referred to in the present invention refers to a thing that stores nutrients for germination of a young plant and is used for agricultural breeding. Specific examples thereof include seeds of corn, soybean, cotton, rice, sugar beet, wheat, barley, sunflower, tomato, cucumber, eggplant, spinach, pea, pumpkin, sugar cane, tobacco, green pepper, oilseed rape, and F1 varieties thereof, Seed potatoes such as aroid, potato, sweet potato, and konjac, bulbs such as edible lily and tulip, seed bulbs such as Chinese scallion, and seeds and tubers of genetically modified crops.

The compound of the present invention has an excellent effect as an active ingredient of a pest control agent or an arthropod pest control agent, and can be used for pests to be controlled, particularly arthropod pests, in various agricultural scenes where a dry field, a paddy field, a tea garden, an orchard, a pasture, a lawn, a forest, a garden, a street tree, or the like is damaged.

By applying the compound of the present invention to plant seeds, it is possible to prevent damage caused by pests such as agricultural pests occurring in plants after seeding.

The compound of the present invention exhibits a control effect on stored grain pests that damage grain and the like stored in a warehouse.

The compound of the present invention exhibits a control effect on insects such as wood pests which damage wood such as buildings, furniture, and stored wood.

The compound of the present invention has an exterminating effect on hygienic pests adversely affecting the living environment of humans such as houses.

The compound of the present invention has an exterminating effect on external or internal parasites of mammals and birds.

The compound of the present invention can control any pest of mites, crustaceans, mollusc, and nematodes which are generated and infested in the same scene as described above.

The compound of the present invention is effective against pests such as arthropods including insects, mites, crustaceans, arachnids and centipedes, mollusc, and nematodes. Specific examples of pest names are shown below, but are not limited thereto.

Examples thereof include Hemiptera pests such as Aleurocanthus camelliae, Aleurocanthus spiniferus, Aleurolobus taonabae, Amrasca biguttulabiguttula, Aonidiella aurantii, Aphis fabae, Aphisglycines, Aphis gossypii, Aphis pomi, Aphis spiraecola, Arboridia apicalis, Aulacorthum solani, Balclutha saltuella, Bemisia argentifolii, Bemisia tabaci, Blissus leucopterus,
Brachycaudus helichrysi, Brevicoryne brassicae, Cacopsylla chinensis, Cacopsylla pyricola, Cacopsylla pyrisuga, Cavelerius saccharivorus, Ceratovacuna lanigera, Ceroplastes ceriferus, Ceroplastes rubens, Cimex hemipterus, Cimex lectularius, Cletus punctiger, Cofana spectra, Comstockaspis perniciosa, Dalbulus maidis, Dialeurodes citri, Diaphorina citri, Dichelops melacanthus, Dolycoris baccarum, Drosicha corpulenta, rosy apple aphid (Dysaphis plantaginea), Dysdercus cingulatus, Empoasca fabae, Empoasca nipponica, Empoasca onukii, Empoasca sakaii, Epiacanthus stramineus, Eriosoma lanigerum, Eurydema rugosum, Euschistus heros, Eysarcoris aeneus, Eysarcoris lewisi, Eysarcoris ventralis,
Fiorinia theae, Glaucias subpunctatus, Halticus insularis, Halyomorpha halys, Hyalopterus pruni, Icerya purchasi, Javesella pellucida, Laodelphax striatella, Leptocorisa acuta, Leptocorisa chinensis, Leptoglossus australis, Lipaphis erysimi, Lygus lineolaris, Lygus lineolaris, Macrosiphum euphorbiae, Macrosteles striifrons), Mahanarva funbriolata, Mahanarva posticata, Melanaphis sacchari, Myzus persicae, Nasonovia ribisnigri, Nephotettix cinctinceps, Nephotettix nigropictus, Nephotettix virescens, Nezara antennata,
Nezara viridula, Nilaparvata lugens, Oebalus pugnax, Peregrinus maidis, Perkinsiella saccharicida, Phenacoccus solani, Phenacoccus solenopsis, Phylloxera devastatrix, Phylloxera notabilis, Phylloxera russelae, Piezodorus guildinii, Piezodorus hybneri, Planococcus citri, Planococcus kuraunhiae, Plautia crossota, Pseudaonidia duplex, Pseudaonidia paeoniae, Pseudaulacaspis pentagona, Pseudaulacaspis prunicola, Pseudococcus calceolariae, Pseudococcus comstocki, Pseudococcus longispinus, Psuedatomoscelis seriatus, Psylla pyrisuga, Pulvinaria aurantii, Quesada gigas, Recilia dorsalis, Rhopalosiphum maidis, Rhopalosiphum padi, Rhopalus msculatus, Riptortus clavatus, Scaptocoris castanea, Scotinophora lurida, Sitobion avenae,
Sogatella furcifera, Stenodema sibiricum, Stenotus rubrovittatus, Stephanitis nashi, Stephanitis pyrioides, Tagosodes orizicolus, Togo hemipterus, Toxoptera aurantii, Toxoptera citricida, Trialeurodes vaporariorum, Triatoma dimidiata, Triatoma infestans, Triatoma rubrofasciata, Trigonotylus caelestialium, Trioza erytreae, Unaspis citri,
Unaspis euonymi, Unaspis yanonensis, and Daktulosphaira Vitifoliae;
Lepidoptera pests such as Acrolepiopsis sapporensis, Acrolepiopsis suzukiella, Adoxophyes honmai, Adoxophyes orana faciata, Adris tyrannus, Aedia leucomelas, Agrotis ipsilon, Agrotis segetum, Alabama argillacea, Anarsia lineatella, Anticarsia gemmatalis, Archips breviplicanus, Archips fuscocupreanus, Argyresthia conjugella, Ascotis selenaria, Autographa nigrisigna, Bucculatrix pyrivorella, Caloptilia theivora, Caloptilia zachrysa, Carposina niponensis, Carposina sasakii, Chilo polychrysus, Chilo suppressalis, Choristoneura magnanima, Cnaphalocrocis medinalis, Conogethes punctiferalis, Ctenoplusia agnata, Cydia pomonella, Diaphania indica, Diatraea saccharalis, Elasmopalpus lignosellus, Endopiza viteana, Ephestia elutella, Etiella zinckenella, Euproctis pseudoconspersa, Glyphodes pyloalis, Grapholita dimorpha, Grapholita inopinata, Grapholita molesta, Helcystogramma triannulella, Helicoverpa armigera, Helicoverpa assulta, Helicoverpa zea, Heliothis virescens, Hellula undalis, Homona magnanima, Hyphantria cunea, Leguminivora glycinivorella, Lemyra imparilis, Leucinodes orbonalis, Leucoptera coffeella, Lobesia botrana, Lymantria dispar, Lyonetia clerkella, Lyonetia prunifoliella malinella, Mamestra brassicae, Manduca quinquemaculata, Manduca sexta, Matsumuraeses phaseoli, Mythimna separata, Naranga aenescens, Nymphula depunctalis, Olethreutes mori, Orgyia thyellina, Ostrinia furnacalis, Ostrinia nubilalis, Ostrinia scapulalis, Pandemis heparana, Papilio xuthus, Parapediasia teterrella, Pamara guttata, Pectinophora gossypiella, Phthorimaea operculella, Phyllocnistis citrella, Phyllonorycter ringoniella, Pieris brassicae, Pieris rapae, Plodia interpunctella, Plusia festucae, Plutella xylostella, Pseudoplusia includens, Scirpophaga incertulas, Scirpophaga innotata, Sesamia inferens, Spodoptera depravata, Spodoptera eridania, Spodoptera exigua, Spodoptera frugiperda, Spodoptera littoralis, Spodoptera litura, Spodoptera mauritia, Spulerrina astaurota, Stathmopoda masinissa, Synanthedon tenuis, Tinea translucens, Trichoplusia ni, and Tuta absoluta;
Coleoptera pests such as Agriotes fuscicollis, Alphitobius diaperinus, Anomala cuprea, Anomala rufocuprea, Anoplophora malasiaca, Anthonomus grandis, Anthrenus verbasci, Aromia bungii, Aulacophora femoralis, Callosobruchus chinensis, Cerotoma trifurcata, Chaetocnema concinna, Chaetocnema confinis, Chaetocnema pulicaria, Cryptolestes ferrugineus, Cylas formicarius, Dermestes maculates, Diabrotica balteata, Diabrotica barberi, Diabrotica speciosa, Diabrotica undecimpunctata, Diabrotica virgifera, Diabrotica virgifera zeae,
Dicladispa armigera, Diloboderus abderus, Echinocnemus squameus, Epilachna varivestis, Epilachna varivestis, Epilachna vigintioctopunctata, Epitrix hirtipennis, Epuraea domina, Euscepes postfasciatus, Gametis jucunda, Heptophylla picea, Hypera postica, Hypothenemus hampei, Lasioderma serricorne, Leptinotarsa decemlineata, Lissorhoptrus oryzophilus, Lyctusbrunneus,
Melanotus fortnumi, Melanotus legatus, Melanotus okinawensis, Melanotus tamsuyensis, Migdolus fryanus, Monochamus alternatus, Oryzaephilus surinamensis, Otiorhynchus sulcatus, Oulema melanopus, Oulema oryzae, Paederus fuscipes, Phyllophaga crinita, Phyllotreta cruciferae, Phyllotreta pusilla, Phyllotreta striolata, Popillia japonica, Psacothea hilaris, Psylliodes angusticollis, Psylliodes chrysocephala, Psylliodes punctulata, Rhizopertha dominica, Rhynchites heros, Sitophilus granarius, Sitophilus oryzae, Sitophilus zeamais, Sphenophorus callosus, Sphenophorus levis, Sphenophorus venatus vestitus, Stegobium paniceum, Sternechus subsignatus, Tenebrio molitor, Tomicus piniperda, Tribolium castaneum, Tribolium confusum, Trogoderma granarium, Xylotrechus pyrrhoderus;
Thysanoptera pests such as Frankliniella intonsa, Frankliniella occidentalis, Haplothrips aculeatus, Heliothrips haemorrhoidalis, Ponticulothrips diospyrosi, Scirtothrips dorsalis, Thrips flavus, Thrips palmi, and Thrips tabaci;
Diptera pests such as Aedes aegypti, Aedes albopicutus, Agromyza oryzae, Anopheles albimanus, Anopheles arabiensis, Anopheles coluzzii, Anopheles darlingi, Anopheles farauti, Anopheles funestus, Anopheles gambiae, Anopheles minimus, Anopheles sinesis, Anopheles stephensi, Anopheles sundaicus, Asphondylia yushimai, Bactrocera cucurbitae, Bactrocera dorsalis, Bactrocera latifrons, Bactrocera oleae, Bactrocera tryoni, Bradysia difformis, Ceratitis capitata, Chironomus plumosus, Chironomus yoshimatsui, Chlorops oryzae, Chromatomyia horticola, Clogmia albipunctata, Culex pipiens molestus, Culex pipiens pallens, Culex pipiens pipiens, Culex quinquefasciatus, Culex vishnui, Delia antique, Delia platura, Drosophila melanogaster, Drosophila suzukii, Glossina morsitans, Glossina palpalis, Haematobia irritans,
Hydrellia griseola, Hydrellia philippina, Hydrellia sasakii, Hypoderma bovis, Hypoderma lineatum, Leptoconops nipponensis, Liriomyza bryoniae, Liriomyza chinensis, Liriomyza sativae, Liriomyza trifolii, Mayetiola destructor, Melophagus ovinus, Musca domestica, Muscina stabulans, Oestrus ovis, Pegomya cunicularia, Prosimulium yezoensis, Rhagoletis pomonella, Simulium damnosum, Simulium japonicum, Sitodiplosis mosellana, Stomoxys calcitrans, Tabanus trigonus, Telmatoscopus albipunctatus, Tetanops myopaeformis, Tipula aino, Tipula oleracea, and Tipula paludosa;
Hymenoptera pests such as Apethymus kuri, Arge pagana, Athalia japonica, Athalia rosae, Atta capiguara, Camponotus japonicus, Camponotus obscuripes, Dryocosmus kuriphilus, Eciton burchelli, Eciton schmitti, Linepithema humile, Monomorium pharaonis, Myrmecia spp., Neodiprion sertifer, Polistes jokahamae, Solenopsis geminata, Solenopsis invicta, Solenopsis spp., Urocerus gigas, Vespa analis, Vespa mandarina, Vespa simillima, and Vespa velutina;
Orthoptera pests such as Acheta domestica, Gryllotalpa orientalis, Locusta migratoria, Melanoplus bivittatus, Melanoplus differentialis, Melanoplus femurrubrum, Melanoplus sanguinipes, Oxya japonica, Oxya yezoensis, Schistocerca gregaria, and Teleogryllus emma;
Collembola pests such as Bourletiella hortensis, Onychiurus folsomi, and Onychiurus sibiricus;
Blattodea pests such as Blatta orientalis, Blattella germanica, Coptotermes formosanus, Coptotermes guangzhouensis, Cornitermes cumulans, Odontotermes formosanus, Periplaneta Americana, Periplaneta australasiae, Periplaneta brunnea, Periplaneta fuliginosa, Periplaneta japonica, Reticulitermes amamianus, Reticulitermes kanmonensis, Reticulitermes miyatakei, and Reticulitermes speratus;
Sihonaptera pests such as Ctenocephalides canis, Ctenocephalides felis, Echidnophaga gallinacea, Pulex irritans, Tunga penetrans, and Xenopsylla cheopis;
Psocodea pests such as Bovicola bovis, Bovicola ovis, Damalinia forficula, Felicola subrostratus, Haematopinus eurystemus, Haematopinus suis, Linognathus ovillus, Linognathus vituli, Liposcelis bostrychophila, Liposcelis corrodens, Liposcelis entomophila, Liposcelis pearmani, Menacanthus stramineus, Menopon gallinae, Solenopotes capillatus, and Trichodectes canis;
Acari pests such as Acaphylla theavagrans, Aceria diospyri, Aceria tosichella, Aceria tulipae, Aculops lycopersici, Aculops pelekassi, Aculus schlechtendali, Amblyomma americanum, Amblyomma maculatum, Amblyomma spp., Argas persicus, Boophilus microplus, Brevipalpus phoenicis, Cheyletiella blakei, Cheyletiella yasguri, Demodex canis, Demodex cati, Dermacentor andersoni, Dermacentor occidentalis, Dermacentor recticulatus, Dermacentor spp., Dermacentor taiwanensis, Dermacentor variabilis, Dermanyssus gallinae, Dermatophagoides farinae, Dermatophagoides pteronyssinus, Eriophyes chibaensis, Haemaophysalis campanulata, Haemaophysalis flava, Haemaphysalis japonica, Haemaphysalis longicornis, Haemaphysalis megaspinosa, Ixodes holocyclus, Ixodes nipponensis, Ixodes ovatus, Ixodes pacifcus, Ixodes pacificus, Ixodes persulcatus, Iodes ricinus, Ixodes scapularis,
Knemidocoptes spp., Listrophorus gibbus, Notoedres cati, Notoedres muris, Oligonychus shinkajii, Ornithonyssus bacoti, Ornithonyssus sylviarum, Otodectes cynotis, Panonychus citri, Panonychus mori, Panonychus ulmi, Penthaleus erythrocephalus, Penthaleus major, Phyllocoptruta citri, Phyllocoptruta oleivora, Phytonemus pallidus, Polyphagotarsonemus latus, Psoroptes equi, Psoroptes ovis, Rhipicephalus annulatus,
Rhipicephalus appendiculatus, Rhipicephalus decoloratus, Rhipicephalus sanguineus, Rhizoglyphus robini, Sarcoptes scabiei, Tarsonemus bilobatus, Tetranychus evansi, Tetranychus kanzawai, Tetranychus urticae, Tyrophagus putrescentiae, Tyrophagus similis, and Varroa jacobsoni;
Thysanura pests such as Ctenolepisma villosa and Lepisma saccharina;
Araneae pests such as Latrodectus hasseltii;
crustacean pests Armadillidium vulgare; gastropod pests such as Achatina fulica, Acusta despecta sieboldiana, Euhadra peliomphala, Limax Valentiana, Meghimatium bilineatum, and Pomacea canaliculata;
Nematoda pests such as Aphelenchoides besseyi, Bursaphelenchus xylophilus, Ditylenchus dipsaci, Globodera rostochiensis, Heterodera glycines, guava root-knot nematodes (Meloidogyne enterolobii), Meloidogyne hapla, Meloidogyne incognita, Meloidogyne javanica, Prathylenchus coffeae, Prathylenchus penetrans, Prathylenchus vulnus, Pratylenchus brachyurus, Pratylenchus coffeae, Pratylenchus neglectus, Radopholus similis, Rotylenchulus reniformis, Tylenchulus semipenetrans, and Xiphinema index, but the present invention is not limited thereto.

The pest is preferably an arthropod pest, more preferably a Hemiptera pest or a Lepidoptera pest, and particularly preferably Nilaparvata lugens, Myzus persicae, Spodoptera litura, or Sogatella furcifera.

The compound of the present invention has a remarkable control effect on the pests that damage paddy field crops, dry field crops, fruit trees, vegetables, other crops, flowers and ornamental plants, and the like, and the effect as the pest control agent of the present invention is obtained by treating paddy field crops, dry field crops, fruit trees, vegetables, other crops, paddy field water such as flowers and ornamental plants, foliage, or soil before the occurrence of pests or at the time when the occurrence of pests is confirmed in accordance with the time when the occurrence of pests is predicted.

The compound of the present invention has a remarkable control effect on grain storing pests and the like generated during storage of a harvested product. That is, a pest control agent or an arthropod pest control agent containing the compound of the present invention as an active ingredient may be applied to a harvested product or a preservation place of the harvested product by a post-harvest treatment such as spraying, smearing, coating, dipping, powder coating, fumigation/smoking, or pressurized injection.

By applying the compound of the present invention to plant seeds, it is possible to prevent damage caused by pests such as agricultural pests occurring in plants after seeding. That is, the plant seeds may be subjected to the treatment such as spraying, smearing, dipping or powder coating with an amount effective for pest control of the pest control agent or the arthropod pest control agent containing the compound of the present invention as an active ingredient as it is or in the form of being appropriately diluted or suspended with water or the like, and thereby the compound of the present invention may be brought into contact with the plant seeds.

The compound of the present invention has a remarkable control effect on wood pests such as termites, Lyctus brunneus, Rhizopertha dominica, Anobiidae, and a longicorn beetle, and the wood pests can be controlled by treating wood such as soil or buildings.

The compound of the present invention exhibits a control effect on various pests or arthropod pests, and exhibits an excellent control effect as an insecticide or a miticide at a low dosage together with an effect of protecting useful crops, and thus has an effect of greatly contributing to a reduction in load on the environment. Furthermore, the compound of the present invention exhibits an excellent control effect by being mixed with other an agricultural or horticultural insecticide, a miticide, a nematicide, a bactericide, an herbicide, a plant growth regulator, an agricultural chemical, and the like.

The compound of the present invention may be used alone, but can be preferably mixed with a solid carrier, a liquid carrier, a gas carrier, a surfactant, a fixing agent, a dispersant, a stabilizer, or the like, and used as a composition of a dustable powder, a wettable powder, a water dispersible granule, a water soluble powder, a water soluble granule, a granule, an emulsifiable concentrate, a soluble concentrate, a microemulsion, an aqueous suspension formulation, an aqueous emulsion formulation, a suspoemulsion, or the like. The composition is not limited thereto as long as the effect is exhibited.

Specific formulation examples are shown below, but the formulation examples are not limited thereto.

The compound of the present invention may be used alone, but is preferably mixed with an appropriate solid carrier or liquid carrier, and further added with a surfactant, a penetrant, a spreading agent, a thickener, an anti-freezing agent, a binder, an anti-caking agent, a disintegrant, a defoaming agent, an antiseptic agent, an anti-decomposition agent, and the like as desired, and as a result, the compound can be put into practical use in a pharmaceutical composition in any dosage form such as a soluble concentrate, an emulsifiable concentrate, a wettable powder, a water soluble powder, a water dispersible granule, a water soluble granule, a suspension concentrate, a concentrated emulsion, a suspoemulsion, a microemulsion, a dustable powder, a granule, a tablet, and an emulsifiable gel. In addition, from the viewpoint of labor saving and improvement of safety, a formulation of any of the above dosage forms can be enclosed in a water-soluble package such as a water-soluble capsule and a bag of a water-soluble film. The composition is not limited thereto as long as the effect is exhibited.

The inert carrier that can be used in the present invention may be either a solid or a liquid, and examples of a material that can be used as a solid inert carrier include soybean power, grain power, wood power, bark powder, saw powder, tobacco stem powder, walnut shell powder, bran, fiber powder, residues after extracting plant extracts, a synthetic polymer such as a pulverized synthetic resin, clays (for example, kaolin, bentonite, acid clay, and the like), talcs (for example, talc, pyrophyllite, and the like), silicas (for example, diatomaceous earth, silica sand, mica, and white carbon [highly dispersed synthetic silicic acid, also known as hydrated fine powder silicon or hydrated silicic acid, and some products contain calcium silicate as a main component], inorganic mineral powders such as activated carbon, sulfur powder, pumice, calcined diatomaceous earth, crushed bricks, fly ash, sand, calcium carbonate, and calcium phosphate, chemical fertilizers such as ammonium sulfate, ammonium phosphorus, ammonium nitrate, urea, and ammonium chloride, and compost, which are used alone or in the form of a mixture of two or more kinds thereof.

A material that can be used as a liquid inter carrier is selected from materials that themselves have solvent ability, as well as materials that do not have solvent ability but can disperse the active ingredient compound with aid of an auxiliary agent, for example, the following carriers can be exemplified as representative examples and used alone or in the form of a mixture of two or more kinds thereof, and examples thereof include water, alcohols (for example, methanol, ethanol, isopropanol, butanol, ethylene glycol, and the like), ketones (for example, acetone, methyl ethyl ketone, methyl isobutyl ketone, diisobutyl ketone, cyclohexanone, and the like), ethers (for example, diethyl ether, dioxane, cellosolve, diisopropyl ether, tetrahydrofuran, and the like), aliphatic hydrocarbons (for example, kerosene, mineral oil, and the like), aromatic hydrocarbons (for example, benzene, toluene, xylene, solvent naphtha, alkylnaphthalene, and the like), halogenated hydrocarbons (for example, dichloromethane, chloroform, carbon tetrachloride, chlorobenzene, and the like), esters (for example, ethyl acetate, butyl acetate, ethyl propionate, diisobutyl phthalate, dibutyl phthalate, dioctyl phthalate, and the like), amides (for example, dimethylformamide, diethylformamide, dimethylacetamide, and the like), and nitriles (for example, acetonitrile and the like). These solid and liquid carriers may be used alone or in combination of two or more thereof.

Examples of the surfactant include nonionic surfactants such as polyoxyethylene alkyl ether, polyoxyethylene alkyl (mono- or di)phenyl ether, polyoxyethylene (mono-, di-, or tri)styrylphenyl ether, a polyoxyethylene polyoxypropylene block copolymer, a polyoxyethylene fatty acid (mono- or di)ester, sorbitan fatty acid ester, polyoxyethylene sorbitan fatty acid ester, a castor oil ethylene oxide adduct, acetylene glycol, acetylene alcohol, an ethylene oxide adduct of acetylene glycol, an ethylene oxide adduct of acetylene alcohol, and alkyl glycosides, anionic surfactants such as alkyl sulfate ester salt, alkylbenzene sulfonate, lignin sulfonate, alkyl sulfosuccinate, naphthalene sulfonate, alkylnaphthalene sulfonate, a salt of a formalin condensate of naphthalene sulfonic acid, a salt of a formalin condensate of alkylnaphthalene sulfonic acid, polyoxyethylene alkyl ether sulfate or phosphate ester salt, polyoxyethylene (mono- or di)alkylphenyl ether sulfate or phosphate ester salt, polyoxyethylene (mono-, di- or tri)styrylphenyl ether sulfate or phosphate ester salt, polycarboxylate salts (for example, polyacrylates, polymaleates, copolymers of maleic acid and olefins, and the like), and polystyrene sulfonate, cationic surfactants such as alkylamine salt and alkyl quaternary ammonium salt, amphoteric surfactants such as amino acid type and betaine type, silicone-based surfactants, and fluorine-based surfactants.

A content of these surfactants is not particularly limited, and is desirably usually in a range of 0.05 to 20 parts by weight with respect to 100 parts by weight of the formulation of the present invention. In addition, these surfactants may be used alone or in combination of two or more thereof.

An application rate and an application concentration of the composition containing the compound of the present invention vary depending on various factors, for example, a purpose, a target pest, a growth situation of crops, an occurrence tendency of pests, weather, environmental conditions, a dosage form, an application method, an application site, an application time, and the like, and in general, the amount of the active ingredient is appropriately 0.0001 to 5,000 ppm, and is preferably used at a concentration of 0.01 to 1,000 ppm. In addition, the application rate per 10a is generally preferably used at a concentration of 1 to 300 g of the active ingredient. In addition, the amount of the active ingredient used in the case of the seed treatment is 0.0001 to 1,000 g, and preferably 0.001 to 100 g, per 1 kg of seeds. When the composition containing the compound of the present invention is used as a foliage spraying treatment onto a plant individual, a spraying treatment onto a soil surface, an injection treatment into soil, or a soil irrigation treatment, the treatment may be performed after the composition is diluted with an appropriate carrier at an appropriate concentration. When the composition containing the compound of the present invention is brought into contact with a plant seed, the composition may be diluted to an appropriate concentration, then immersed in the plant seed, dusted, sprayed or smeared, and used. The amount of the composition used in the case of immersion, powder coating, spraying, or smearing treatment is usually about 0.05 to 50% of the dry plant seed weight as the amount of the active ingredient, and preferably and appropriately 0.1 to 30%. However, the amount may be appropriately set depending on the form of the composition and the type of plant seed to be treated, and is not limited to these ranges.

The amount of the active ingredient of the compound of the present invention represented by Formula (1-1) or Formula (1-2) is 0.1 to 90 wt %, preferably 1 to 90 wt %, more preferably 1 to 50 wt %, and particularly preferably 3 to 50 wt %. The amount of the active ingredient of the compound of the present invention represented by Formula (1-1) or Formula (1-2) are usually 0.1 to 20 wt % in a dustable powder, 5 to 50 wt % in an emulsifiable concentrate, 3 to 90 wt % in a wettable powder, 0.1 to 20 wt % in a granule, 5 to 90 wt % in a flowable formulation, and 3 to 90 wt % in a water dispersible granule. On the other hand, the amount of the carrier in each of the dosage forms is usually 60 to 99.9 wt % in a dustable powder, 40 to 95 wt % in an emulsifiable concentrate, 10 to 90 wt % in a wettable powder, 80 to 99.9 wt % in a granule, 10 to 95 wt % in a flowable formulation, and 10 to 90 wt % in a water dispersible granule. In addition, the amount of the auxiliary agent is usually 0.1 to 20 wt % in a dustable powder, 1 to 20 wt % in an emulsifiable concentrate, 0.1 to 20 wt % in a wettable powder, 0.1 to 20 wt % in a granule, 0.1 to 20 wt % in a flowable formulation, and 0.1 to 20 wt % in a water dispersible granule.

The composition containing the compound of the present invention can be used by being mixed with another agricultural chemical as necessary, for example, an agricultural chemical such as a bactericide, an insecticide, a miticide, a nematicide, an herbicide, a biological agricultural chemical, or a plant growth regulator, a disease control agent containing a nucleic acid as an active ingredient (WO 2014/062775 A), a soil conditioner, or a fertilizer. Examples of the method for mixing and using the compound of the present invention and another agricultural chemical include a method in which the compound of the present invention and another agricultural chemical are formulated into one dosage form for use, a method in which both of them formulated in separate dosage forms are mixed and used before use, a method in which both of them formulated in separate dosage forms are used simultaneously, and a method in which either one of them is used and then the other is used for both of them formulated in separate dosage forms.

The components contained in the insecticide are as shown in the following group b, and include salts, isomers, and N-oxides thereof. However, the known insecticides are not limited thereto.

Group b:

### <b-1: Carbamate-Based Acetylcholinesterase (AChE) Inhibitor>

b-1: Examples of the carbamate-based acetylcholinesterase (AChE) inhibitor include [b-1.1] phosphocarb, [b-1.2] alanycarb, [b-1.3] butocarboxim, [b-1.4] butoxycarboxim, [b-1.5] thiodicarb, [b-1.6] thiofanox, [b-1.7] aldicarb, [b-1.8] bendiocarb, [b-1.9] benfuracarb, [b-1.10] carbaryl, [b-1.11] carbofuran, [b-1.12] carbosulfan, [b-1.13] ethiofencarb, [b-1.14] fenobucarb, [b-1.15] formetanate, [b-1.16] furathiocarb, [b-1.17] isoprocarb, [b-1.18] methiocarb, [b-1.19] methomyl, [b-1.20] oxamyl, [b-1.21] pirimicarb, [b-1.22] propoxur, [b-1.23] trimethacarb, [b-1.24] XMC (3,5-xyly1 methylcarbamate), [b-1.25] allyxycarb, [b-1.26] aldoxycarb, [b-1.27] bufencarb, [b-1.28] butacarb, [b-1.29] carbanolate, [b-1.30] metolcarb, [b-1.31] xylylcarb, and [b-1.32] fenothiocarb.

### <b-2: Organophosphorus Acetylcholinesterase (AChE) Inhibitor>

b-2: Examples of the organophosphorus acetylcholinesterase (AChE) inhibitor include [b-2.1] acephate, [b-2.2] azamethiphos, [b-2.3] azinphos-methyl, [b-2.4]azinphos-ethyl, [b-2.5] ethephon, [b-2.6] cadusafos, [b-2.7] chlorethoxyfos, [b-2.8] chlorfenvinphos, [b-2.9] chlormephos, [b-2.10] chlorpyrifos, [b-2.11] chlorpyrifos-methyl, [b-2.12] coumaphos, [b-2.13] cyanophos, [b-2.14] demeton-S-methyl, [b-2.15] diazinon, [b-2.16] dichlofenthion, [b-2.17] dichlorvos, [b-2.18] dicrotophos, [b-2.19] dimethoate, [b-2.20] dimethylvinphos, [b-2.21] disulfoton, [b-2.22] O-ethyl 0-4-nitrophenyl phenylphosphonothioate, [b-2.23] ethion, [b-2.24] ethoprophos, [b-2.25] famphur, [b-2.26] fenamiphos, [b-2.27] fenitrothion, [b-2.28] fenthion, [b-2.29] fosthiazate, [b-2.30] heptenophos, [b-2.31] isofenphos-methyl, [b-2.32] isocarbophos, [b-2.33] isoxathion, [b-2.34] malathion, [b-2.35] mecarbam, [b-2.36] methamidophos, [b-2.37] methidathion, [b-2.38] mevinphos, [b-2.39] monocrotophos, [b-2.40] naled, [b-2.41] omethoate, [b-2.42] oxydemeton-methyl, [b-2.43] parathions, [b-2.44] parathion-methyl, [b-2.45] phenthoate, [b-2.46] phorate, [b-2.47] phosalone, [b-2.48] phosmet, [b-2.49] phosphamidon, [b-2.50] phoxim, [b-2.51] pirimiphos-methyl, [b-2.52] profenofos, [b-2.53] propetamphos, [b-2.54] prothiofos, [b-2.55] pyraclofos, [b-2.56] pyridaphenthion, [b-2.57] quinalphos, [b-2.58] sulfotep, [b-2.59] tebupirimfos, [b-2.60] temephos, [b-2.61] terbufos, [b-2.62] thiometon, [b-2.63] triazophos, [b-2.64] trichlorfon, [b-2.65] vamidothion, [b-2.66] chlorothion, [b-2.67] bromfenvinfos,

[b-2.68] bromophos, [b-2.69] bromophos-ethyl, [b-2.70] butathiofos, [b-2.71] carbophenothion, [b-2.72] chlorphoxim, [b-2.73] sulprofos, [b-2.74] diamidafos, [b-2.75] tetrachlorvinphos, [b-2.76] propaphos, [b-2.77] mesulfenfos, [b-2.78] dioxabenzofos, [b-2.79] etrimfos, [b-2.80] oxydeprofos, [b-2.81] formothion, [b-2.82] fensulfothion, [b-2.83] isazofos, [b-2.84] imicyafos, [b-2.85] isamidofos, [b-2.86] thionazin, and [b-2.87] fosthietan.

### <b-3: Cyclic Diene Organochlorine GABAergic Chloride Ion Channel Blocker>

b-3: Examples of the cyclic diene organochlorine GABAergic chloride ion channel blocker include [b-3.1] chlordane, [b-3.2] endosulfan, [b-3.3] lindane), and [b-3.4] dienochlor.

### <b-4: Phenylpyrazole-Based GABAergic Chloride Ion Channel Blocker>

b-4: Examples of the phenylpyrazole-based GABAergic chloride ion channel blocker include [b-4.1] ethiprole, [b-4.2] fipronil, and [b-4.3] acetoprole.

### <b-5: Sodium Channel Modulator>

b-5: Examples of the sodium channel modulator include [b-5.1] acrinathrin, [b-5.2] allethrin, [b-5.3] bifenthrin, [b-5.4] bioallethrin, [b-5.5] bioallethrin S-cyclopentenyl isomer, [b-5.6] bioresmethrin, [b-5.7] cycloprothrin, [b-5.8] cyfluthrin, [b-5.9] beta-cyfluthrin, [b-5.10] cyhalothrin, [b-5.11] gamma-cyhalothrin, [b-5.12] lambda-cyhalothrin, [b-5.13] cypermethrin, [b-5.14] alpha-cypermethrin, [b-5.15] beta-cypermethrin, [b-5.16] theta-cypermethrin, [b-5.17] zeta-cypermethrin, [b-5.18] cyphenothrin [(1R)-trans-isomer], [b-5.19] deltamethrin, [b-5.20] empenthrin [(EZ)-(1R)-isomer], [b-5.21] esfenvalerate, [b-5.22] ethofenprox, [b-5.23] fenpropathrin, [b-5.24] fenvalerate, [b-5.25] flucythrinate, [b-5.26] flumethrin, [b-5.27] tau-fluvalinate, [b-5.28] halfenprox, [b-5.29] imiprothrin, [b-5.30] methothrin, [b-5.31] metofluthrin, [b-5.32] epsilon-metofluthrin, [b-5.33] momfluorothrin, [b-5.34] epsilon-momfluorothrin, [b-5.35] permethrin, [b-5.36] phenothrin [(1R)-trans-isomer], [b-5.37] prallethrin, [b-5.38] pyrethrin, [b-5.39] resmethrin, [b-5.40] kadethrin, [b-5.41] silafluofen, [b-5.42] tefluthrin, [b-5.43] tetramethrin, [b-5.44] tetramethrin [(1R)-isomer], [b-5.45] tralomethrin, [b-5.46] transfluthrin, [b-5.47] ZX18901 (3-(4-bromophenoxy)phenyl]-cyanomethyl 4-(difluoromethoxy)-α-(1-methylethyl)benzeneacetate), [b-5.48] biopermethrin, [b-5.49] furamethrin, [b-5.50] profluthrin, [b-5.51] flubrocythrinate, [b-5.52] dimefluthrin, [b-5.53] phenothrin, [b-5.54] fluvalinate, [b-5.55] dichloro-diphenyl-trichloroethane (DDT), and [b-5.56]methoxychlor.

<b-6: Nicotinic Acetylcholine Receptor (nAChR) Competitive Modulator>

b-6: Examples of the nicotinic acetylcholine receptor (nAChR) competitive modulator include [b-6.1] acetamiprid, [b-6.2] clothianidin, [b-6.3] dinotefuran, [b-6.4] imidacloprid, [b-6.5] nitenpyram, [b-6.6] thiacloprid, [b-6.7] thiamethoxam, [b-6.8] nicotine, [b-6.9] nicotine sulfate, [b-6.10] sulfoxaflor, [b-6.11] flupyradifurone, [b-6.12] triflumezopyrim, and [b-6.13] flupyrimin.

<b-7: Nicotinic Acetylcholine Receptor (nAChR) Allosteric Modulator-Site I>

b-7: Examples of the nicotinic acetylcholine receptor (nAChR) allosteric modulator-site I include [b-7.1] spinosad and [b-7.2] spinetoram.

### <b-8: Glutamatergic Chloride Channel (GluCl) Allosteric Modulator>

b-8: Examples of the glutamatergic chloride ion channel (GluCl) allosteric modulator include [b-8.1] abamectin, [b-8.2] emamectin benzoate, [b-8.3] lepimectin, and [b-8.4] milbemectin.

### <b-9: Juvenile Hormone Analogue>

b-9: Examples of the juvenile hormone analogue include [b-9.1] hydroprene, [b-9.2] kinoprene, [b-9.3] methoprene, [b-9.4] fenoxycarb, and [b-9.5] pyriproxyfen.

<b-10: Other Non-Specific (Multisite) Inhibitor>

b-10: Examples of the other non-specific (multisite) inhibitor include [b-10.1] methyl bromide, [b-10.2] chloropicrin, [b-10.3] cryolite, [b-10.4] sulfuryl fluoride, [b-10.5] borax, [b-10.6] boric acid, [b-10.7] disodium octaborate, [b-10.8] sodium metaborate, [b-10.9] tartar emetic, [b-10.10] dazomet, [b-10.11] metam, and [b-10.12] metham sodium.

<b-11: Chordotonal Organ TRPV Channel Modulator>

b-11: Examples of the chordotonal organ TRPV channel modulator include [b-11.1] pymetrozine, [b-11.2] pyrifluquinazon, and [b-11.3] afidopyropen.

<b-12: Mite Growth Inhibitor That Acts on CHS1>

b-12: Examples of the mite growth inhibitor that acts on CHS1 include [b-12.1] clofentezine, [b-12.2] diflovidazin, [b-12.3] hexythiazox, and [b-12.4] etoxazole.

<b-13: Microorganism-Derived Insect Midgut Membrane Disrupting Agent>

b-13: Examples of the microorganism-derived insect midgut membrane disrupting agent include [b-13.1] Bacillus thuringiensis, [b-13.2] B.t. subsp. Israelensis, [b-13.3] B.t. subsp. Aizawai, [b-13.4] B.t. subsp. kurstaki, [b-13.5] B.t. subsp. tenebrionis, [b-13.6] B.t. crop proteins: Cry1Ab, Cry1Ac, Cry1Fa, Cry1A.105, Cry2Ab, Vip3A, mCry3A, Cry3Ab, Cry 3Bb, and Cry34Ab1/Cry35Ab1, and [b-13.7] Bacillus sphaericus.

<b-14: Mitochondrial ATP Synthase Inhibitor>

b-14: Examples of the mitochondrial ATP synthase inhibitor include [b-14.1] diafenthiuron, [b-14.2] azocyclotin, [b-14.3] cyhexatin, [b-14.4] fenbutatin oxide, [b-14.5] propargite, and [b-14.6] tetradifon.

<b-15: Oxidative Phosphorylation Uncoupler that Perturbs Proton Gradient>

b-15: Examples of the oxidative phosphorylation uncoupler that perturbs a proton gradient [b-15.1] chlorfenapyl, [b-15.2] dinitro-ortho-cresol (DNOC), [b-15.3] binapacryl, and [b-15.4] sulfluramid.

<b-16: Nicotinic Acetylcholine Receptor (nAChR) Channel Blocker>

b-16: Examples of the nicotinic acetylcholine receptor (nAChR) channel blocker include [b-16.1] bensultap, [b-16.2] cartap hydrochloride, [b-16.3] thiocyclam, [b-16.4] thiosultap, [b-16.5] Thiosultap-sodium, and [b-16.6] monosultap.

<b-17: Chitin Biosynthesis Inhibitor That Acts on CHS1>

b-17: Examples of the chitin biosynthesis inhibitor that acts on CHS1 include [b-17.1] bistrifluron, [b-17.2] chlorfluazuron, [b-17.3] diflubenzuron, [b-17.4] flucycloxuron, [b-17.5] flufenoxuron, [b-17.6] hexaflumuron, [b-17.7] lufenuron, [b-17.8] novaluron, [b-17.9] noviflumuron, [b-17.10] teflubenzuron, and [b-17.11] triflumuron.

<b-18: Chitin Biosynthesis Inhibitor Type 1>

b-18: Examples of the chitin biosynthesis inhibitor type 1 include [b-18.1] buprofezin.

<b-19: Diptera Insect Molting Inhibitor>

b-19: Examples of the diptera insect molt inhibitor include [b-19.1] cyromazine.

<b-20: Moulting Hormone (Ecdysone) Receptor Agonist>

b-20: Examples of the molting hormone (ecdysone) receptor agonist include [b-20.1] chromafenozide, [b-20.2] halofenozide, [b-20.3] methoxyfenozide, and [b-20.4] tebufenozide.

<b-21: Octopamine Receptor Agonist>

b-21: Examples of the octopamine receptor agonist include [b-21.1] amitraz.

<b-22: Mitochondrial Electron Transport Chain Complex III Inhibitor>

b-22: Examples of the mitochondrial electron transport chain complex III inhibitor include [b-22.1] hydramethylnon, [b-22.2] acequinocyl, [b-22.3] fluacrypyrim, and [b-22.4] bifenazate.

<b-23: Mitochondrial Electron Transport Chain Complex Inhibitor (METI)>

b-23: Examples of the mitochondrial Electron Transport Chain Complex Inhibitor (METI) include [b-23.1] fenazaquin, [b-23.2] fenpyroximate, [b-23.3]pyridaben, [b-23.4] pylimidifen, [b-23.5] tebufenpyrad, [b-23.6] tolfenpyrad, and [b-23.7] rotenone).

<b-24: Potential-Dependent Sodium Channel Blocker>

b-24: Examples of the potential-dependent sodium channel blocker include [b-24.1] indoxacarb and [b-24.2] metaflumizone.

<b-25: Acetyl CoA Carboxylase Inhibitor>

b-25: Examples of the acetyl CoA carboxylase inhibitor include [b-25.1] spirodiclofen, [b-25.2] spiromesifen, [b-25.3] spiropidion, and [b-25.4] spirotetramat.

<b-26: Mitochondrial Electron Transport Chain Complex IV Inhibitor>

b-26: Examples of the mitochondrial electron transport chain complex IV inhibitor include [b-26.1] aluminum phosphide, [b-26.2] calcium phosphide, [b-26.3] phosphine, [b-26.4] zinc phosphide, [b-26.5] calcium cyanide, [b-26.6] potassium cyanide, and [b-26.7] sodium cyanide.

<b-27: Mitochondrial Electron Transport Chain Complex II Inhibitor>

b-27: Examples of the mitochondrial electron transport chain complex II inhibitor include [b-27.1] cyenopyrafen, [b-27.2] cyflumetofen, and [b-27.3] pyflubumide.

<b-28: Ryanodine Receptor Modulator>

b-28: Examples of the ryanodine receptor modulator include [b-28.1] chlorantraniliprole, [b-28.2] cyantraniliprole, [b-28.3] flubendiamide, [b-28.4]cyclaniliprole, and [b-28.5] tetraniliprole.

<b-29: Chordotonal Organ Modulator Target Site Unidentified>

b-29: Examples of the choronic organ modulator target site unspecified insecticide include [b-29.1] flonicamid.

<b-30: GABAergic Chloride Ion Channel Allosteric Modulator>

b-30: Examples of the GABAergic chloride ion channel allosteric modulator include [b-30.1] broflanilide and [b-30.2] fluxametamide.

<b-31: Baculovirus>

b-31: Examples of the baculovirus include [b-31.1] Cydia pomonella (GV), [b-31.2] Thaumatotibia leucotreta (GV), [b-31.3] Anticarsia gemmatalis (MNPV), and [b-31.4] Helicoverpa armigera (NPV).

<b-32: Nicotinic Acetylcholine Receptor (nAChR) Allosteric Modulator-Site II>

b-32: Examples of the nicotinic acetylcholine receptor (nAChR) allosteric modulator-Site II include [b-32.1] GS-omega/kappa HXTX-Hvla peptide.

<b-33: Other Insecticides>

b-33: Examples of the other insecticides [b-33.1] azadirachtin, [b-33.2]benzoximate, [b-33.3] phenisobromolate, [b-33.4] chinomethionat, [b-33.5] dicofol, [b-33.6] lime sulfur mixture (CaSx), [b-33.7] manzeb, [b-33.8] pyridalyl, [b-33.9] sulfur, [b-33.10] bromopropylate, [b-33.11] (Burkholderia spp, Wolbachia pipientis (Zap)), [b-33.12] Chenopodium ambrosioides near ambrosioides extract, [b-33.13] Fatty acid monoesters with glycerol or propanediol, [b-33.14] Neem oil, [b-33.15] Beauveria bassiana strains, [b-33.16] Metarhizium anisopliae strain F52, [b-33.17] Paecilomyces fumosoroseus Apopka strain 97, [b-33.18] diatomite, [b-33.19] triazamate, [b-33.20] dicyclanil, [b-33.21] dinobuton, [b-33.22] dinocap, [b-33.23] hydrogen cyanide, [b-33.24] methyl iodide, [b-33.25] karanjin, [b-33.26] mercury chloride, [b-33.27] methyl isothiocyanate, [b-33.28] pentachlorophenol, [b-33.29] phosphine, [b-33.30] piperonyl butoxide, [b-33.31] polynactins, [b-33.32] sabadilla, [b-33.33] sulcofuron-sodium, [b-33.34] tribufos, [b-33.35] aldrin, [b-33.36] amidithion, [b-33.37] amidothioate, [b-33.38] aminocarb, [b-33.39] amiton, [b-33.40] aramite, [b-33.41] athidathion, [b-33.42] azothoate, [b-33.43] barium polysulphide, [b-33.44] benclothiaz, [b-33.45] 5-1,3-benzodioxole-5-yl-3-hexylcyclohexa-2-enone, [b-33.46] 1,1-bis 4-chlorophenyl-2-ethoxyethanol, [b-33.47] butonate, [b-33.48] butopyronoxyl, [b-33.49] 2-2-butoxyethoxy ethyl thiocyanate, [b-33.50] camphechlor, [b-33.51] chlorbenside, [b-33.52] chlordecone, [b-33.53] chlordimeform, [b-33.54] chlorfenethol, [b-33.55] chlorfenson, [b-33.56] fluazuron, [b-33.57] metaldehyde, [b-33.58] bialaphos, [b-33.59] levamisol, [b-33.60] amidoflumet, [b-33.61] pyrafluprole, [b-33.62] pyriprole, [b-33.63] tralopyril, [b-33.64] flupyrazofos, [b-33.65] diofenolan, [b-33.66] chlorobenzilate, [b-33.67] flufenzine, [b-33.68] benzomate, [b-33.69] flufenerim, [b-33.70] albendazole, [b-33.71] oxibendazole, [b-33.72] fenbendazole, [b-33.73] metam-sodium, [b-33.74] 1,3-dichloropropene, [b-33.75] ethylene dibromide, [b-33.76] acrylonitrile, [b-33.77] bis (2-chloroethyl)ether, [b-33.78] 1-bromo-2-chloroethane, [b-33.79] 3-bromo-1-chloroprop-1-ene, [b-33.80] bromocyclen, [b-33.81] carbon disulfide, [b-33.82] tetrachloromethane, [b-33.83] nemadectin, [b-33.84] cymiazole, [b-33.85] cytokinin, [b-33.86] 2-(octylthio)ethanol, [b-33.87] potassium oleate, [b-33.88] sodium oleate, [b-33.89] machine oil, [b-33.90] tar oil, [b-33.91] anabasine, [b-33.92] morantel tartrate, [b-33.93] Pyrethrum, [b-33.94] rape seed oil, [b-33.95] soybean lecithin, [b-33.96] starch, [b-33.97]hydroxypropylstarch,

[b-33.98] decanoyloctanoylglycerol, [b-33.99] diatomite, [b-33.100] tripropyl isocyanurate (TPIC), [b-33.101] D-D(1,3-Dichloropropene), [b-33.102] peroxocarbonate, [b-33.103] MB-599 (verbutin), [b-33.104] bis(2,3,3,3-tetrachloropropyl) ether), [b-33.105] bis(2-chloro-1-methylethyl)ether (DCIP), [b-33.106] ENT-8184 (N-(2-Ethylhexyl)bicyclohept-5-ene-2,3-dicarboximide), [b-33.107] Bayer 22408 (O,O-diethyl O-naphthalimido phosphorothioate), [b-33.108] Bayer 32394 (tris(1-dodecyl-3-methyl-2-phenylbenzimidazolium)hexacyanoferrate), [b-33.109] flometoquin, [b-33.110] dicloromezotiaz, [b-33.111] fluazaindolizine, [b-33.112] cyhalodiamide, [b-33.113] tioxazafen, [b-33.114] fluhexafon, [b-33.115] fluralaner, [b-33.116] tetrachlorantraniliprole, [b-33.117] sarolaner, [b-33.118] lotilaner, [b-33.119] tigolaner, [b-33.120] cycloxaprid, [b-33.121] fluensulfone, [b-33.122] benzpyrimoxan, [b-33.123] acynonapyr, [b-33.124] fenmezoditiaz, [b-33.125] tyclopyrazoflor, [b-33.126] oxazosulfyl, [b-33.127] isocycloseram, [b-33.128] dimpropyridaz, [b-33.129] cyproflanilide, [b-33.130] nicofluprole, [b-33.131] cyclobutrifluram, [b-33.132] cycloxylidin, [b-33.133] paichongding, [b-33.134] guadipyr, [b-33.135] cyetpyrafen, [b-33.136] flupentiofenox, [b-33.137] pyriminostrobin, [b-33.138] chloroprallethrin, [b-33.139] kappa-bifenthrin, [b-33.140] kappa-tefluthrin, [b-33.141] heptafluthrin,
a compound represented by [b-33.142] Formula (s1)

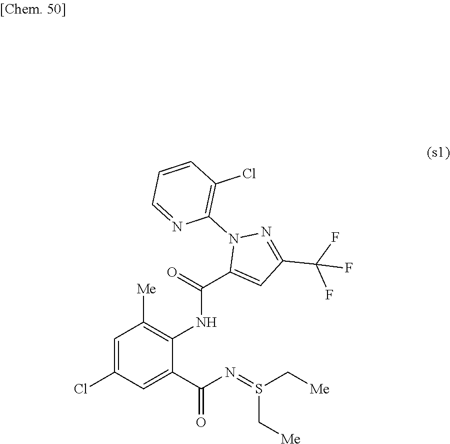 [SMILES: CCS(CC)=NC(=O)c1cc(Cl)cc(C)c1NC(=O)c1cc(C(F)(F)F)nn1-c1ncccc1Cl]

(see WO 2020/018362 A),
a compound represented by [b-33.143] Formula (s2)

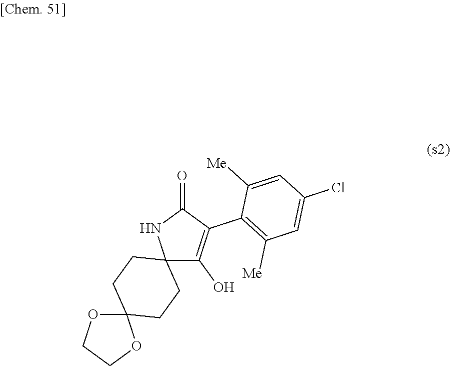 [SMILES: Cc1cc(Cl)cc(C)c1C1=C(O)C2(CCC3(CC2)OCCO3)NC1=O]

(see WO 2006/089633 A),
a compound represented by [b-33.144] Formula (s3)

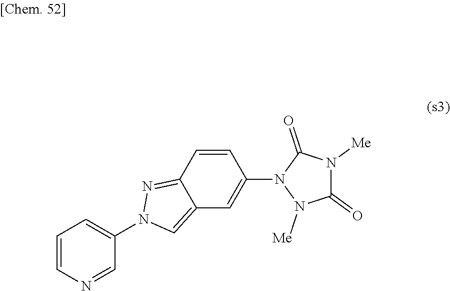

(see WO 2018/011111 A),
a compound represented by [b-33.145] Formula (s4)

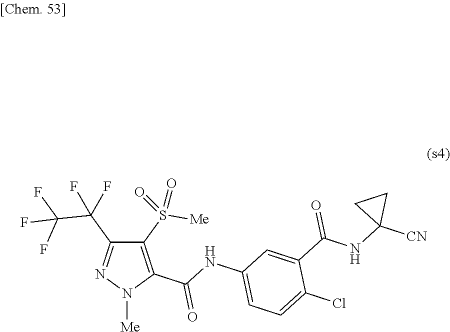 [SMILES: Cn1nc(C(F)(F)C(F)(F)F)c(S(C)(=O)=O)c1C(=O)Nc1ccc(Cl)c(C(=O)NC2(C#N)CC2)c1]

(see WO 2012/126766 A),
a compound represented by [b-33.146] Formula (s5)

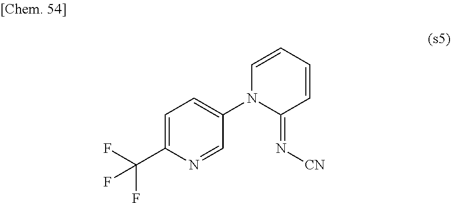 [SMILES: N#C/N=c1\ccccn1-c1ccc(C(F)(F)F)nc1]

(see WO 2019/215076 A),
a compound represented by [b-33.147] Formula (s6)

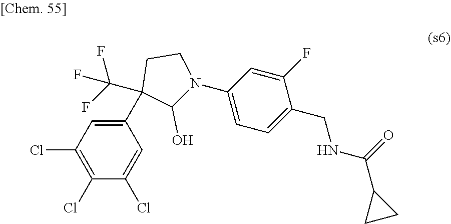 [SMILES: O=C(NCc1ccc(N2CCC(c3cc(Cl)c(Cl)c(Cl)c3)(C(F)(F)F)C2O)cc1F)C1CC1]

(see WO 2016/180802 A),
a compound represented by [b-33.148] Formula (s7)

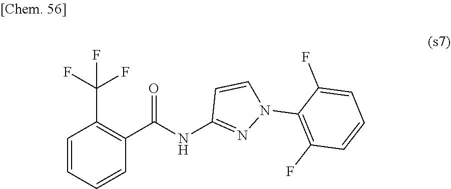 [SMILES: O=C(Nc1ccn(-c2c(F)cccc2F)n1)c1ccccc1C(F)(F)F]

(see WO 2014/053450 A),
a compound represented by [b-33.149] Formula (s8)

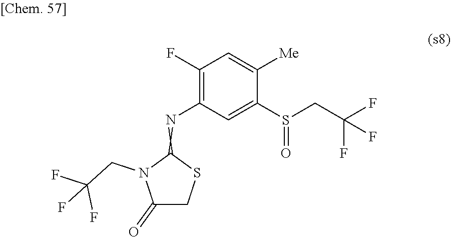 [SMILES: Cc1cc(F)c(N=C2SCC(=O)N2CC(F)(F)F)cc1S(=O)CC(F)(F)F]

(see WO 2013/092350 A),
a compound represented by [b-33.150] Formula (s9)

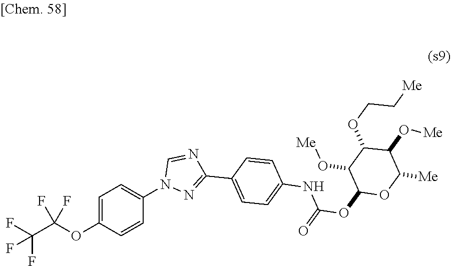

(see WO 2012/027521 A),
a compound represented by [b-33.151] Formula (s10)

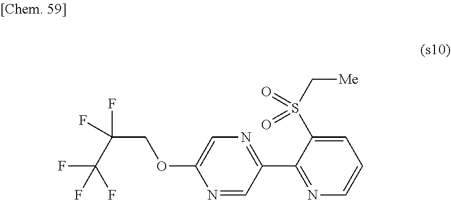 [SMILES: CCS(=O)(=O)c1cccnc1-c1cnc(OCC(F)(F)C(F)(F)F)cn1]

(see WO 2017/065228 A),
a compound represented by [b-33.152] Formula (s11)

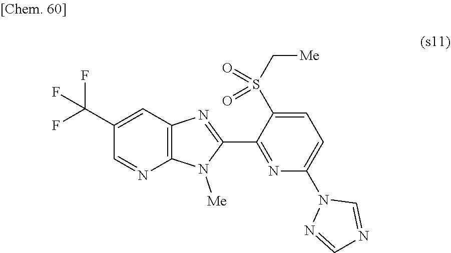 [SMILES: CCS(=O)(=O)c1ccc(-n2cncn2)nc1-c1nc2cc(C(F)(F)F)cnc2n1C]

(see WO 2015/133603 A),

a compound represented by [b-33.153] Formula (s12)

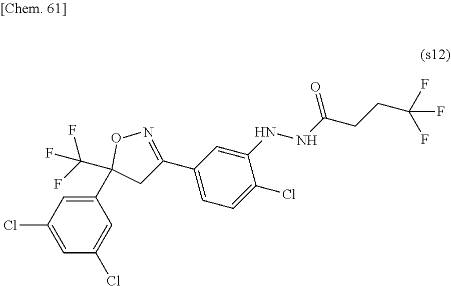 [SMILES: O=C(CCC(F)(F)F)NNc1cc(C2=NOC(c3cc(Cl)cc(Cl)c3)(C(F)(F)F)C2)ccc1Cl]

(see WO 2010/090344 A),
a compound represented by [b-33.154] Formula (s13)

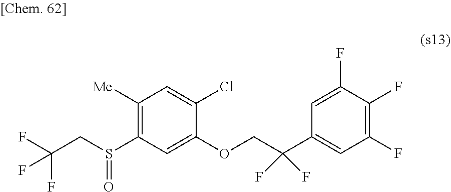 [SMILES: Cc1cc(Cl)c(OCC(F)(F)c2cc(F)c(F)c(F)c2)cc1S(=O)CC(F)(F)F]

(see WO 2015/025826 A), and
a compound represented by [b-33.155] Formula (s14)

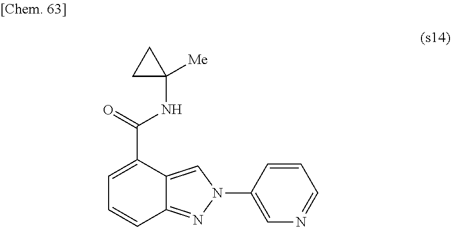 [SMILES: CC1(NC(=O)c2cccc3nn(-c4cccnc4)cc23)CC1]

(see WO 2016/144351 A).

The components contained in the bactericide are as shown in the following group c, and include salts, isomers, and N-oxides thereof. However, the known bactericides are not limited thereto.

### Group c:

### <c-1: Phenylamide-Based Bactericide>

c-1: Examples of the phenylamide-based bactericide include [c-1.1] benalaxyl, [c-1.2] benalaxyl-M or kiralaxyl, [c-1.3] furalaxyl, [c-1.4] metalaxyl, [c-1.5] metalaxyl-M or mefenoxam, [c-1.6] oxadixyl, and [c-1.7] ofurace.

### <c-2: Hydroxy(2-Amino)Pyrimidine-Based Bactericide>

c-2: Examples of the hydroxy(2-amino)pyrimidine-based bactericide include [c-2.1] bupirimate, [c-2.2] dimethirimol, and [c-2.3] ethirimol.

### <c-3: Aromatic Eeterocyclic Bactericide>

c-3: Examples of the aromatic heterocyclic bactericide include [c-3.1] hymexazole and [c-3.2] oxolinic acid.

### <c-4: Carboxylic Acid Bactericide>

c-4: Examples of the carboxylic acid bactericide include [c-4.1] octhilinone.

### <c-5: β-Tubulin Polymerization Inhibitor>

c-5: Examples of the β-tubulin polymerization inhibitor include [c-5.1] benomyl, [c-5.2] carbendazim, [c-5.3] fuberidazole, [c-5.4] thiabendazole, [c-5.5]thiophanate, [c-5.6] thiophanate-methyl, [c-5.7] diethofencarb, [c-5.8] zoxamide, and [c-5.9] ethaboxam.

### <c-6: Cell Division Inhibitor>

c-6: Examples of the cell division inhibitor include [c-6.1] pencycuron.

c-7: Delocalization of Spectrin-Like Protein

c-7: Examples of the bactericide related to delocalization of spectrin-like protein include [c-7.1] fluopicolide and [c-7.2] fluopimomide.

c-8: Actin/Myosin/Fibrin Function

c-8: Examples of the bactericide related to the actin/myosin/fibrin function include [c-8.1] Phenamacril, [c-8.2] metrafenone, and [c-8.3] pyriopenone.

### <c-9: Complex I: NADH Oxidoreductase Inhibitor>

c-9: Complex I: Examples of the NADH oxidoreductase inhibitor include [c-9.1] diflumetorim, [c-9.2] tolfenpyrad, and [c-9.3] fenazaquin.

<c-10: SDHI Agent (Succinate Dehydrogenase Inhibitor)>

c-10: Examples of the SDHI agent (succinate dehydrogenase inhibitors) include [c-10.1] benodanil, [c-10.2] benzovindiflupyr, [c-10.3] bixafen, [c-10.4] boscalid, [c-10.5] carboxin, [c-10.6] fenfuram, [c-10.7] fluopyram, [c-10.8] flutolanil, [c-10.9] fluxapyroxad, [c-10.10] furametpyr, [c-10.11] isofetamid, [c-10.12] isopyrazam, [c-10.13] mepronil, [c-10.14] oxycarboxin, [c-10.15] penthiopyrad, [c-10.16] penflufen, [c-10.17] pydiflumetofen, [c-10.18] sedaxane, [c-10.19] thifluzamide, [c-10.20] pyraziflumid, [c-10.21] isoflucypram, [c-10.22] fluindapyr, [c-10.23] inpyrfluxam.

<c-11: QoI Agent (Quinone External Inhibitor)>

c-11: Examples of the QoI agent (quinone external inhibitor) include [c-11.1] azoxystrobin, [c-11.2] coumoxystrobin, [c-11.3] dimoxystrobin, [c-11.4] enoxastrobin, [c-11.5] famoxadone, [c-11.6] fenamidone, [c-11.7] fenaminstrobin, [c-11.8] flufenoxystrobin, [c-11.9] fluoxastrobin, [c-11.10] kresoxim-methyl, [c-11.11] mandestrobin, [c-11.12] metominostrobin, [c-11.13] orysastrobin, [c-11.14] picoxystrobin, [c-11.15] pyraclostrobin, [c-11.16] pyrametostrobin, [c-11.17] pyraoxystrobin, [c-11.18] pyribencarb, [c-11.19] triclopyricarb, [c-11.20] trifloxystrobin, and [c-11.21] metyltetraprole.

<c-12: QiI Agent (Quinone Internal Inhibitor)>

c-12: Examples of the QiI agent (quinone internal inhibitor) include [c-12.1] amisulbrom, [c-12.2] cyazofamid, and [c-12.3] fenpicoxamid.

<c-13: Oxidative Phosphorylation Uncoupling Inhibitor>

c-13: Examples of the oxidative phosphorylation uncoupling inhibitor include [c-13.1] binapacryl, [c-13.2] meptyldinocap, [c-13.3] dinocap, [c-13.4] fluazinam, and [c-13.5] ferimzone.

<c-14: ATP Synthase Inhibitor>

c-14: Examples of the ATP synthase inhibitor include [c-14.1] fentin acetate, [c-14.2] fentin chloride, and [c-14.3] fentin hydroxide.

<c-15: ATP Transport>

c-15: Examples of the bactericide related to ATP transport include [c-15.1] silthiofam.

<c-16: QoSI Agent (Quinone External Stigmaterin-Binding Subsite Inhibitor)>

c-16: Examples of the QoSI agent (quinone external stigmaterin-binding subsite inhibitor) include [c-16.1] ametoctradin.

<c-17: Methionine Biosynthesis Inhibitor>

c-17: Examples of the methionine biosynthesis inhibitor include [c-17.1] cyprodinil, [c-17.2] mepanipyrim, and [c-17.3] pyrimethanil.

<c-18: Protein Biosynthesis Inhibitor>

c-18: Examples of the protein biosynthesis inhibitor include [c-18.1] blasticidin-S, [c-18.2] kasugamycin, [c-18.3] streptomycin, and [c-18.4] oxytetracycline.

<c-19: Signal Transduction Inhibitor>

c-19: Examples of the signal transduction inhibitor include [c-19.1] quinoxyfen, [c-19.2] proquinazid, [c-19.3] fenpiclonil, [c-19.4] fludioxonil, [c-19.5] chlozolinate, [c-19.6] dimethachlone, [c-19.7] iprodione, [c-19.8] procymidone, and [c-19.9] vinclozolin.

<c-20: Lipid and Cell Membrane Biosynthesis Inhibitor>

c-20: Examples of the lipid and cell membrane biosynthesis inhibitor include [c-20.1] edifenphos, [c-20.2] iprobenfos, [c-20.3] isoprothiolane, [c-20.4] pyrazophos, [c-20.5] biphenyl, [c-20.6] chloroneb, [c-20.7] dicloran, [c-20.8] quintozene, [c-20.9] tecnazene, [c-20.10] tolclofos-methyl, [c-20.11] echlomezol or etridiazole, [c-20.12] iodocarb, [c-20.13] propamocarb, and [c-20.14] prothiocarb.

<c-21: Cell Membrane Disturbance Agent>

c-21: Examples of the bactericide related to the cell membrane disturbance agent include [c-21.1] an extract from Melaleuca alternifolia and [c-21.2] plant oils (mixtures): eugenol, geraniol, and thymol.

<c-22: Ergosterol Binder>

c-22: Examples of the bactericide related to an ergosterol binding include [c-22.1] natamycin.

<c-23: Oxysterol-Binding Protein Inhibitor>

c-23: Examples of the oxysterol-binding protein inhibitor include [c-23.1] oxathiapiprolin and [c-23.2] fluoxapiprolin.

<c-24: DMI Agent (Demethylation Inhibitor)>

c-24: Examples of the DMI agent (demethylation inhibitor) include [c-24.1] azaconazole, [c-24.2] bitertanol, [c-24.3] bromuconazole, [c-24.4] cyproconazole, [c-24.5] difenoconazole, [c-24.6] diniconazole, [c-24.7] diniconazole-M, [c-24.8] epoxiconazole, [c-24.9] etaconazole, [c-24.10] fenarimol, [c-24.11] fenbuconazole, [c-24.12] fluquinconazole, [c-24.13] quinconazole, [c-24.14] flusilazole, [c-24.15] flutriafol, [c-24.16] hexaconazole, [c-24.17] imazalil, [c-24.18] imibenconazole, [c-24.19] ipconazole, [c-24.20] metconazole, [c-24.21] myclobutanil, [c-24.22] nuarimol, [c-24.23] oxpoconazole, [c-24.24] oxpoconazole fumarate, [c-24.25] pefurazoate, [c-24.26] penconazole, [c-24.27] prochloraz, [c-24.28] propiconazole, [c-24.29] prothioconazole, [c-24.30] pyrifenox, [c-24.31] pyrisoxazole, [c-24.32] simeconazole, [c-24.33] tebuconazole, [c-24.34] tetraconazole, [c-24.35] triadimefon, [c-24.36] triadimenol, [c-24.37] triflumizole, [c-24.38] triforine, [c-24.39] triticonazole, [c-24.40] mefentrifluconazole, and [c-24.41] ipfentrifluconazole.

<c-25: Amine-Based Bactericide>

c-25: Examples of the amine-based bactericide include [c-25.1] aldimorph, [c-25.2] dodemorph, [c-25.3] fenpropimorph, [c-25.4] tridemorph, [c-25.5] fenpropidin, [c-25.6] piperalin, and [c-25.7] spiroxamine.

<c-26: 3-Keto Reductase Inhibitor in C4 Demethylation of Sterol Biosynthesis>

c-26: Examples of the 3-keto reductase inhibitor in the C4 demethylation of sterol biosynthesis include [c-26.1] fenhexamid and [c-26.2] fenpyrazamine.

<c-27: Squalene Epoxidase Inhibitor of Sterol Biosynthesis>

c-27: Examples of the squalene epoxidase inhibitor for sterol biosynthesis include [c-27.1] pyributicarb, [c-27.2] naftifine, and [c-27.3] terbinafine.

<c-28: Cell Wall Biosynthesis Inhibitor>

c-28: Examples of the cell wall biosynthesis inhibitor include [c-28.1] polyoxins, [c-28.2] dimethomorph, [c-28.3] flumorph, [c-28.4] pyrimorph, [c-28.5] benthiavalicarb, [c-28.6] benthivalicarb-isopropyl, [c-28.7] iprovalicarb, [c-28.8] mandipropamid, and [c-28.9] valifenalate.

<c-29: Melanin Biosynthesis Inhibitor>

c-29: Examples of the melanin biosynthesis inhibitor include [c-29.1] phthalide or fthalide, [c-29.2] pyroquilone, [c-29.3] tricyclazole, [c-29.4] carpropamid, [c-29.5] diclocymet, [c-29.6] fenoxanil, and [c-29.7] tolprocarb.

<c-30: Host Plant Resistance Inducer>

c-30: Examples of the host plant resistance inducer include [c-30.1] acibenzolar-S-methyl, [c-30.2] probenazole, [c-30.3] tiadinil, [c-30.4] isotianil, [c-30.5] laminarin, [c-30.6] an extract from Reynoutria sachalinensis (giant knotweed), [c-30.7] Bacillus mycoides isolate J, [c-30.8] Cell walls of Saccharomyces cerevisiae strain LAS117), [c-30.9] fosetyl, [c-30.10] phosphorous acid, [c-30.11] sodium phosphite, [c-30.12] ammonium phosphite, and [c-30.13] potassium phosphite.

<c-31: Dithiocarbamate-Based Bactericide>

c-31: Examples of the dithiocarbamate-based bactericide include [c-31.1] mancozeb, [c-31.2] manzeb, [c-31.3] maneb, [c-31.4] metiram, [c-31.5] propineb, [c-31.6] thiram, [c-31.7] zinc thiazole, [c-31.8] zineb, [c-31.9] ziram, and [c-31.10] ferbam.

<c-32: Phthalimide-Based Bactericide>

c-32: Examples of the phthalimide-based bactericide include [c-32.1] captan, [c-32.2] captafol, [c-32.3] folpet, and [c-32.4] fluorofolpet.

<c-33: Guanidine-Based Bactericide>

c-33: Examples of the guanidine-based bactericides include [c-33.1] guazatine, [c-33.2] iminoctadine, [c-33.3] iminoctadine albesilate, and [c-33.4] iminoctadine triacetate.

<c-34: Multi-Action Point Contact Active Bactericide>

c-34: Examples of the multi-action point contact active bactericide include [c-34.1] chlorothalonil, [c-34.2] dichlofluanid, [c-34.3] tolylfluanid, [c-34.4] copper oxychloride, [c-34.5] copper II hydroxide, [c-34.6] copper hydroxide sulfate, [c-34.7] organocopper compound, [c-34.8] dodecylbenzenesulphonic acid bisethylenediamine copper [II] salt, [c-34.9] sulphur, [c-34.10] fluoroimide, [c-34.11] anilazine, [c-34.12] dithianon, [c-34.13] chinomethionat or quinomethionate, and [c-34.14] methasulfocarb.

<c-35: Biological Agricultural Chemical Having Plurality of Action Mechanisms>

c-35: Examples of the biological agricultural chemical having a plurality of action mechanisms include [c-35.1] an extract from cotyledon of lupine plantlets (BLAD), [c-35.2] an extract from Swinglea glutinosa, [c-35.3] Trichoderma atroviride strain 1-1237, [c-35.4] Trichoderma atroviride strain LU132, [c-35.5] Trichoderma atroviride strain SC1, [c-35.6] Trichoderma asperellum strain T34, [c-35.7] Gliocladium catenulatum strain J1446, [c-35.8] Clonostachys rosea strain CR-7, [c-35.9] Bacillus amyloliquefaciens strain QST713, [c-35.10] Bacillus amyloliquefaciens strain FZB24, [c-35.11] Bacillus amyloliquefaciens strain MB1600, [c-35.12] Bacillus amyloliquefaciens strain D747, [c-35.13] Bacillus amyloliquefaciens strain F727, [c-35.14] Bacillus subtilis strain AFS032321, [c-35.15] Pseudomonas chlororaphis strain AFS009, [c-35.16] Streptomyces griseovirides strain K61, and [c-35.17] Streptomyces lydicus strain WYEC108.

<c-36: Other Bactericide>

c-36: Examples of the other bactericides include [c-36.1] tecloftalam, [c-36.2] triazoxide, [c-36.3] flusulfamide, [c-36.4] diclomezine, [c-36.5] cyflufenamid, [c-36.6] dodine, [c-36.7] flutianil, [c-36.8] tebufloquin, [c-36.9] validamycins, [c-36.10] cymoxanil, [c-36.11] picarbutrazox, [c-36.12] quinofumelin, [c-36.13] aminopyrifen, [c-36.14] pyridachlometyl, [c-36.15] ipflufenoquin, [c-36.16] florylpicoxamid, [c-36.17] dichlobentiazox, [c-36.18] pyrapropoyne, [c-36.19] dipymetitrone, [c-36.20] D-tagatose, [c-36.21] mineral oils, organic oils, inorganic salts, material of biological origin, [c-36.22] Chloroinconazide, and [c-36.23] Flubeneteram,
a compound represented by [c-36.24] Formula (s16)

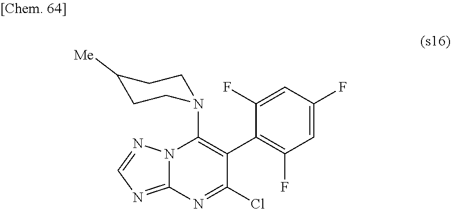 [SMILES: CC1CCN(c2c(-c3c(F)cc(F)cc3F)c(Cl)nc3ncnn23)CC1]

(see WO 98/046607 A),
a compound (gougerotin) represented by [c-36.25] Formula (s17)

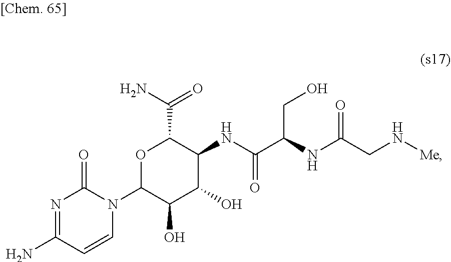 [SMILES: CNCC(=O)N[C@H](CO)C(=O)N[C@H]1[C@H](O)[C@@H](O)C(n2ccc(N)nc2=O)O[C@@H]1C(N)=O]

a compound (ningnanmycin) represented by [c-36.26] Formula (s18)

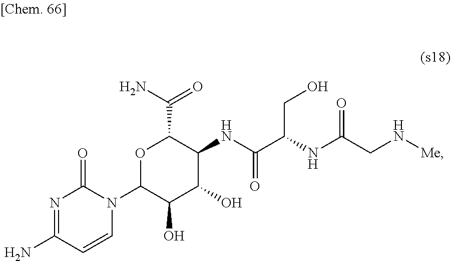 [SMILES: CNCC(=O)N[C@@H](CO)C(=O)N[C@H]1[C@H](O)[C@@H](O)C(n2ccc(N)nc2=O)O[C@@H]1C(N)=O]

a compound represented by [c-36.27] Formula (s19)

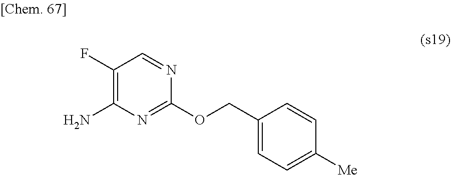 [SMILES: Cc1ccc(COc2ncc(F)c(N)n2)cc1]

(see WO 2011/085084 A),

a compound represented by [c-36.28] Formula (s20)

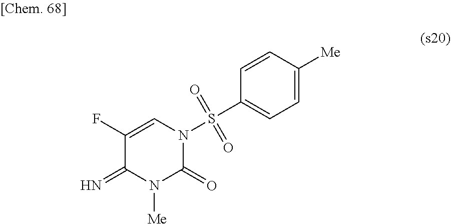 [SMILES: Cc1ccc(S(=O)(=O)n2cc(F)c(=N)n(C)c2=O)cc1]

(see WO 2011/137002 A),
a compound represented by [c-36.29] Formula (s21)

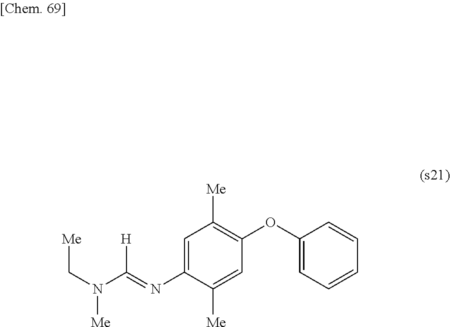 [SMILES: CCN(C)/C=N/c1cc(C)c(Oc2ccccc2)cc1C]

(see WO 2008/110313 A),
a compound represented by [c-36.30] Formula (s22)

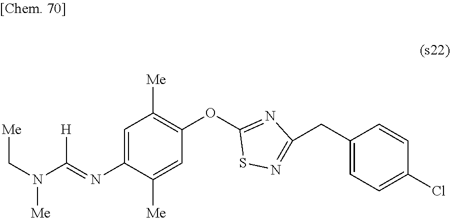 [SMILES: CCN(C)/C=N/c1cc(C)c(Oc2nc(Cc3ccc(Cl)cc3)ns2)cc1C]

(see WO 2009/156098 A),
a compound represented by [c-36.31] Formula (s23)

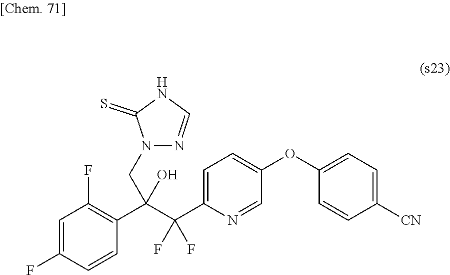 [SMILES: N#Cc1ccc(Oc2ccc(C(F)(F)C(O)(Cn3nc[nH]c3=S)c3ccc(F)cc3F)nc2)cc1]

(see WO 2016/187201 A),
a compound represented by [c-36.32] Formula (s24)

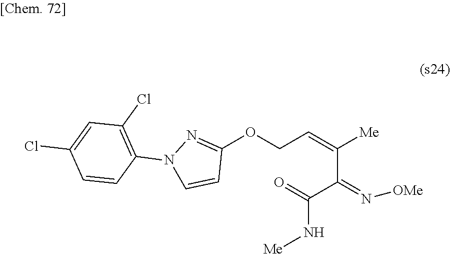 [SMILES: CNC(=O)C(=N/OC)/C(C)=C\COc1ccn(-c2ccc(Cl)cc2Cl)n1]

(see WO 2013/092224 A),
a compound represented by [c-36.33] Formula (s25)

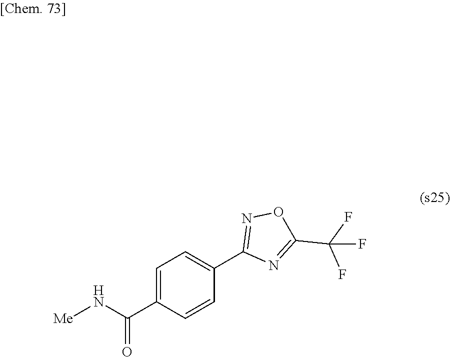 [SMILES: CNC(=O)c1ccc(-c2noc(C(F)(F)F)n2)cc1]

(see WO 2015/185485 A),
a compound represented by [c-36.34] Formula (s26)

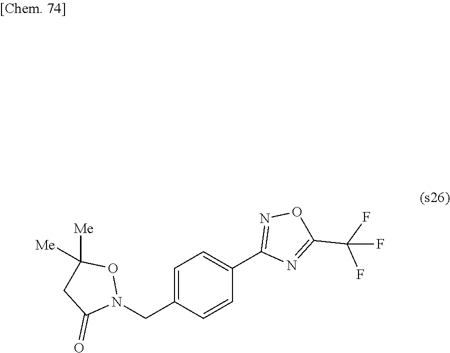 [SMILES: CC1(C)CC(=O)N(Cc2ccc(-c3noc(C(F)(F)F)n3)cc2)O1]

(see WO 2017/093348 A),
a compound represented by [c-36.35] Formula (s27)

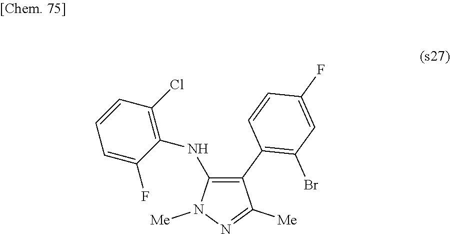 [SMILES: Cc1nn(C)c(Nc2c(F)cccc2Cl)c1-c1ccc(F)cc1Br]

(see WO 2012/031061 A),
compounds represented by [c-36.36] Formula (s28)

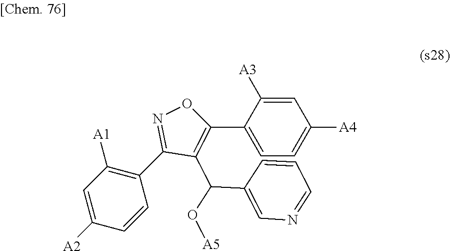

[in the formula, A1, A2, A3 and A4 each independently represent a hydrogen atom or a halogen atom, and A5 represents a hydrogen atom, an acetyl group, or a benzoyl group](see WO 2006/031631 A and WO 2010/069882 A),
compounds represented by [c-36.37] Formula (s29)

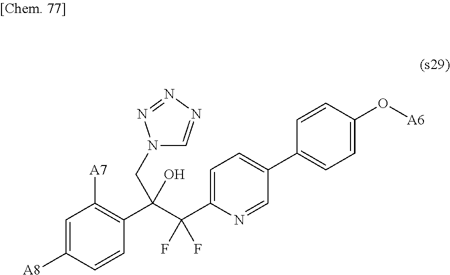

[in the formula, A6 represents a C1 to C6 haloalkyl group, and A7 and A8 each independently represent a hydrogen atom or a halogen atom] (see WO 2014/043376 A),
a compound represented by [c-36.38] Formula (s30)

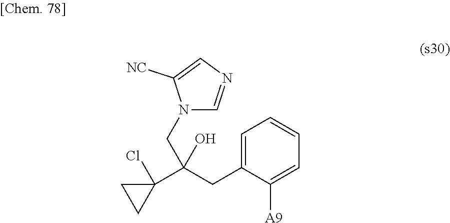

[in the formula, A9 represents a halogen atom] (see WO 2016/156290 A),
a compound represented by [c-36.39] Formula (s31)

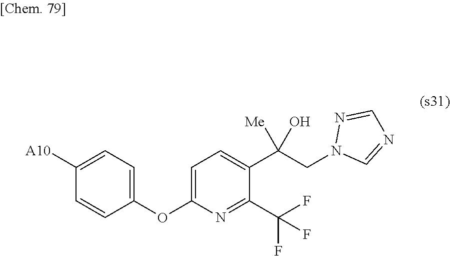

[in the formula, A10 represents a halogen atom] (see WO 2017/029179 A),
a compound represented by [c-36.40] Formula (s32)

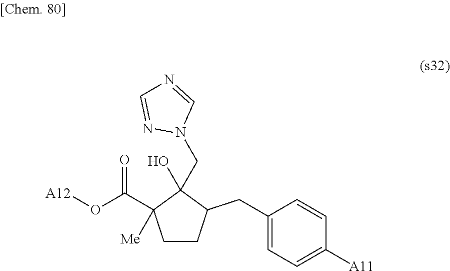

[in the formula, A11 represents a hydrogen atom or a halogen atom, and A12 represents a C1 to C6 alkyl group] (see WO 2011/070771 A),
a compound represented by [c-36.41] Formula (s33)

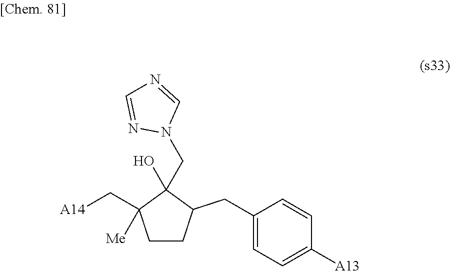

[in the formula, A13 and A14 represent a halogen atom] (see WO 2011/070771 A),
a compound represented by [c-36.42] Formula (s34)

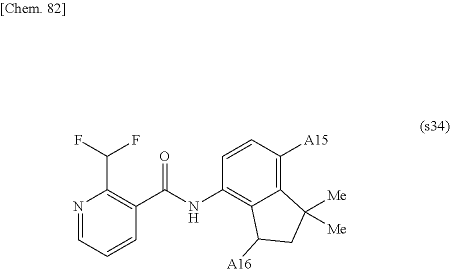

[in the formula, A15 represents a hydrogen atom or a halogen atom, and A16 represents a methyl group, an ethyl group, or a propyl group] (see WO 2014/095675 A),
a compound represented by [c-36.43] Formula (s35)

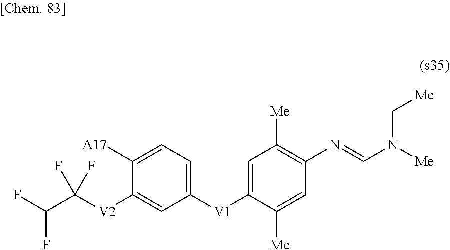

[in the formula, A17 represents a hydrogen atom or a halogen atom, and V1 and V2 each independently represent an oxygen atom or a sulfur atom] (see WO 2012/025450 A),
a compound represented by [c-36.44] Formula (s36)

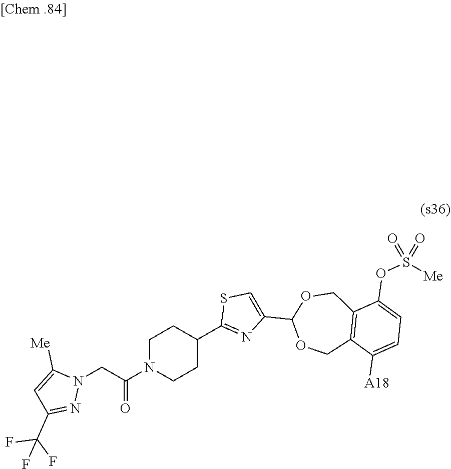

[in the formula, A18 represents a hydrogen atom or a fluorine atom] (see WO 2016/024350 A),
a compound represented by [c-36.45] Formula (s37)

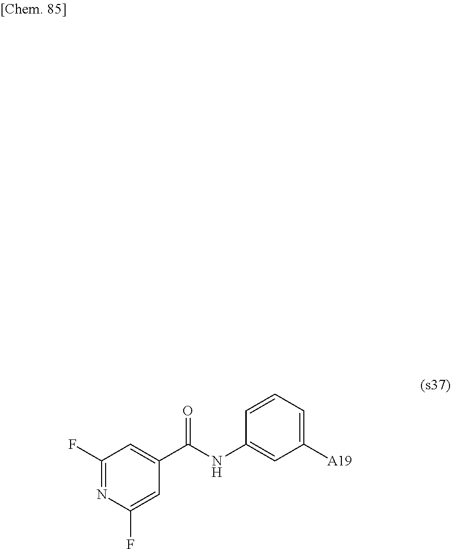

[in the formula, A19 represents a C1 to C6 alkoxy group] (see WO 2018/169038 A),
a compound represented by [c-36.46] Formula (s38)

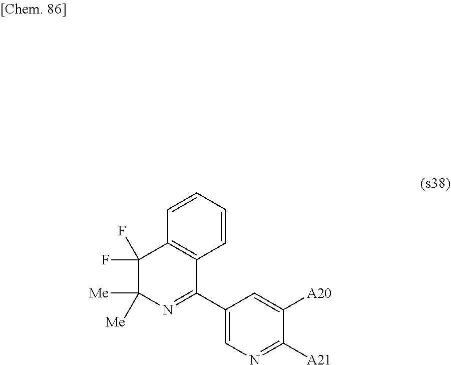

[in the formula, A20 represents a methyl group or a methoxy group, and A21 represents a methyl group or a difluoromethyl group] (see WO 2017/016915 A),
a compound represented by [c-36.47] Formula (s39)

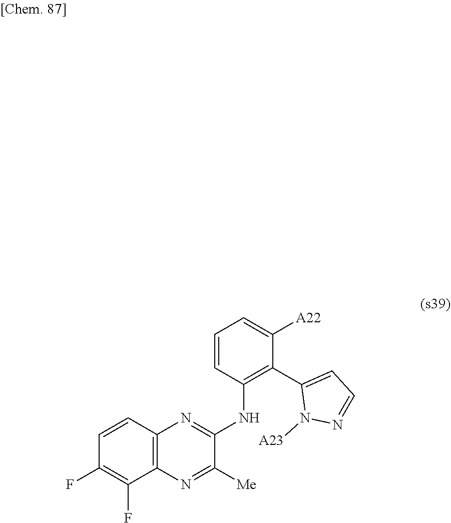

[in the formula, A22 represents a hydrogen atom or a halogen atom, and A23 represents an ethyl group or a propyl group] (see WO 2018/197692 A),
a compound represented by [c-36.48] Formula (s40)

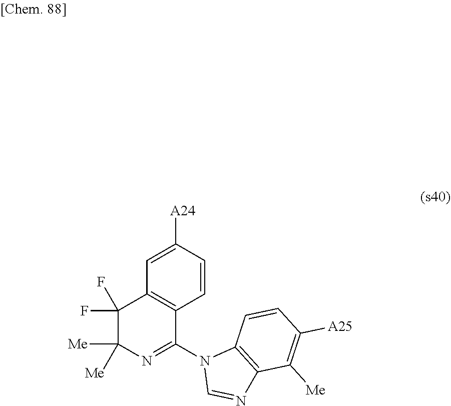

[in the formula, A24 represents a hydrogen atom or a halogen atom, and A25 represents a methyl group, a fluorine atom, or a chlorine atom] (see WO 2016/156085 A),
a compound represented by [c-36.49] Formula (s41)

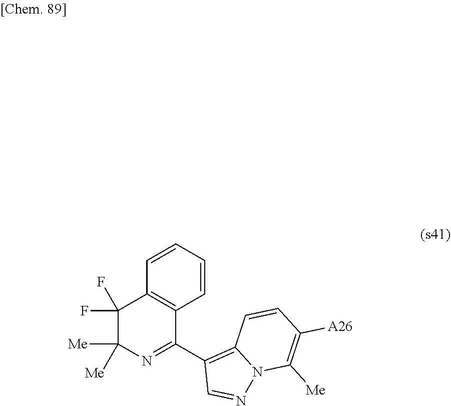

[in the formula, A26 represents a methyl group, a fluorine atom, or a chlorine atom](see WO 2017/025510 A),
a compound represented by [c-36.50] Formula (s42)

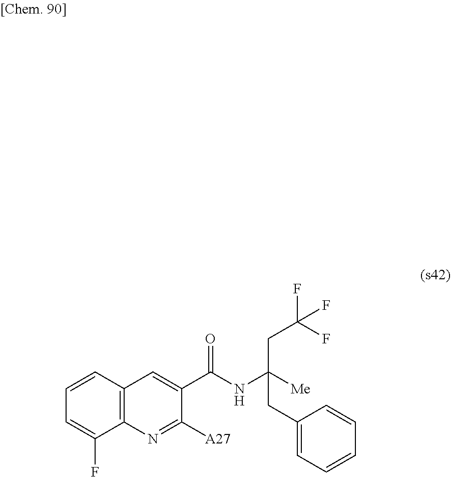

[in the formula, A27 represents a hydrogen atom or a methyl group] (see WO 2017/153380 A),
a compound represented by [c-36.51] Formula (s43)

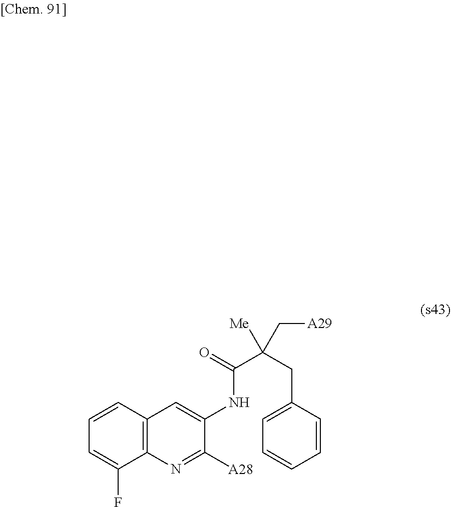

[in the formula, A28 represents a hydrogen atom or a methyl group, and A29 represents an isopropyl group or a trifluoromethyl group] (see WO 2018/172133 A), and
a compound represented by [c-36.52] Formula (s44)

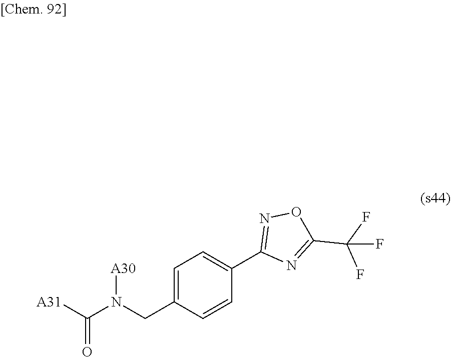

[in the formula, A30 a hydrogen atom or a methoxy group, and A31 represents an ethyl group or a cyclopropyl group] (see WO 2017/055469 A).

A mixing ratio of the compound of the present invention to the other insecticide and bactericide in the mixture of the compound of the present invention, and the other insecticide and bactericide is not particularly limited, and for example, the compound of the present invention:the other insecticide and bactericide is 0.01:100 to 100:0.01, 0.1:100 to 100:0.1, 1:100 to 100:1, 1:10 to 10:1, or 1:1 (all in terms of weight). Further, a range of an arbitrary combination of the upper limit value and the lower limit value is also included as the mixing ratio. When there are two or more other insecticides and bactericides, the above mixing ratio refers to a ratio of the total value of the other insecticides and bactericides to the compound of the present invention.

The compound of the present invention has a remarkable exterminating effect on hygiene pests such as Diptera pests (Culex pipiens pallens, Culex pipiens f. molestus, Chironomidae, Musca domestica, Psychodidae, Tabanus trigonus, and the like) and Blattodea pests (Blattella germanica, Periplaneta fuliginosa, Periplaneta Americana, and the like).

Furthermore, the compound of the present invention can also be used as an anthelmintic for arthropods or the like that directly harm or are mediators of diseases in the surrounding environment in which these pests may be latent by a method such as spraying, injecting, irrigating and applying of an oil agent, an emulsifiable concentrate, a wettable powder and the like; spraying of a dustable powder and the like; a treatment with a fumigant, a mosquito coil, a self-combustion type fumigant, a heated atomizer such as a chemical reaction type atomizer, a smoking agent such as fogging, a ULV agent and the like; installation of granules, tablets and poisonous baits; or dropping of a floating dustable powder, granules and the like into a water passage, a well, a water reservoir, a water storage tank and other flowing water or staying water. Furthermore, it is possible to control Lymantriidae and the like, which are agricultural and forest pests, in the same manner as the method described above, or it is also effective to use a method in which the compound of the present invention is mixed with livestock feed to be mixed with feces for flies and the like, and a method for volatilizing the compound of the present invention into the air with an electric mosquito trap and the like for mosquitoes and the like.

Note that the formulations in these use forms can also be present as a mixture with other active compounds, for example insecticides, miticides, nematicides, bactericides, repellents, or synergists, and it is appropriate that these formulations contain the compound of the present invention in a total amount of, for example, 0.0001 wt % to 95 wt %.

The compound of the present invention can control external or internal parasites of mammals other than human beings and birds such as livestock and pets, such as Ctenocephalides felis, Ctenocephalides canis, Haemaphysalis longicornis, and Filariidae nematodes.

Extermination of external and internal parasites is achieved by oral administration, parenteral administration, or transdermal administration. As oral administration, the compound of the present invention may be mixed in a small amount in a meal, feed, or the like, or may be prepared as an oral administration agent such as an appropriate orally ingestible formulated pharmaceutical composition, for example, an orally administered agent such as a pharmaceutically acceptable carrier, a tablet containing a coating substance, a pill, a capsule, a sustained-release large pill, a paste, a gel, a medicinal beverage, a medicinal feed, medicinal drinking water, an animal feed, a medicinal supplement, or a sustained-release device retained in the gastrointestinal tract, and these agents may be orally administered to livestock and pets.

When administered orally as medicinal drinking water, the medicinal drinking water may be prepared as a solution, suspension, or dispersion in a suitable nontoxic solvent or water, usually together with a suspending agent such as bentonite or a wetting agent or other excipients. In addition, medicinal drinking water generally further contains an antifoaming agent. The medicinal drinking water can generally contain 0.01 wt % to 1.0 wt % and preferably 0.01 wt % to 0.1 wt % of the compound of the present invention.

When oral administration in a unit use form of a dried individual is desirable, capsules, pills, or tablets containing a predetermined amount of the active ingredient are usually used. These use forms are produced by homogeneously mixing the active ingredient with a diluent, a filler, a disintegrant, and/or a binder, for example, starch, lactose, tale, magnesium stearate, vegetable rubber, or the like that are appropriately finely grinded. Such a dry solid unit use formulation can widely vary the mass and content of the anthelmintic depending on the type of host animal to be treated, the extent of infection, and the type of parasite and the weight of the host.

When administered by animal feed, it can be homogeneously dispersed in the feed or used as a top dressing or in pellet form. In order to achieve the normally desired anti-parasitic effect, the compound of the invention can also be contained in the final feed, for example, in an amount of 0.0001 wt % to 0.05 wt % and preferably 0.0005 wt % to 0.01 wt %.

The transdermal administration may be performed transdermally or topically through a transdermal administration agent such as spray, powder, grease, cream, ointment, emulsion, lotion, spot-on, pour-on, or shampoo. As a method of transdermal administration or topical administration, a device attached to an animal so as to control arthropods locally or systemically (for example, a collar, a medallion, an ear tag, or the like) can also be used.

In order to achieve the effect of such transdermal administration or topical administration, the compound of the present invention may be administered in an amount of generally 0.0001 wt % to 0.1 wt % and preferably 0.001 wt % to 0.01 wt %. Note that the compound of the present invention dissolved or dispersed in the liquid carrier excipient can be parenterally administered to an animal by anterior intragastric, intramuscular, intratracheal, or subcutaneous injection. For parenteral administration, the active compound is suitably mixed with a suitable vegetable oil such as peanut oil, cottonseed oil. Such a formulation can generally contain 0.05 wt % to 50 wt % and preferably 0.1 wt % to 5.0 wt % of the compound of the present invention. In addition, the compound of the present invention can be locally administered by mixing with an appropriate carrier such as dimethyl sulfoxide or a hydrocarbon solvent. The formulation is applied directly to the external surface of the animal by spraying or direct injection.

### EXAMPLES

Hereinafter, the present invention will be described in more detail with reference to Synthesis Examples, Reference Examples, and Test Examples, but the present invention is not limited thereto.

### Reference Example 1

### Synthesis of ethyl 3-ethyl-1H-pyrazole-5-carboxylate

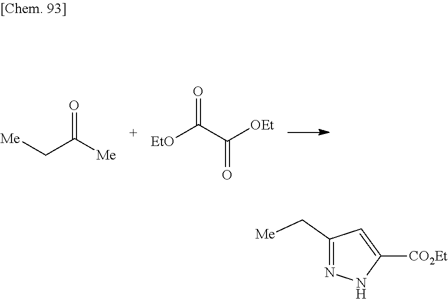 [SMILES: CCC(C)=O ; CCOC(=O)C(=O)OCC ; CCOC(=O)c1cc(CC)n[nH]1]

A mixed solution of 8.90 ml of 2-butanone, 13.5 ml of diethyl oxalate, and 100 ml of ethanol was added to 38.7 ml of sodium ethoxide (20% ethanol solution) at room temperature. Next, the solution was heated to 70° C., and the solution was stirred for 1 hour and 30 minutes. 7.54 g of hydrazine hydrochloride was added to the obtained solution, and the mixture was stirred under heating reflux for 2.5 hours. After cooling to room temperature, water and a saturated sodium bicarbonate aqueous solution were added to the reaction mixture. Ethyl acetate was added to the obtained reaction mixture, and the organic layer was extracted. After separating the organic layer and the aqueous layer, the obtained organic layer was washed with saturated brine and dried with sodium sulfate. After distilling off the solvent under reduced pressure, the obtained residue was purified by silica gel column chromatography. The title compound was obtained as 9.32 g of an orange oil.

¹H-NMR (CDCl₃) δ: 6.63 (1H, s), 4.38 (2H, q, J=7.1 Hz), 2.72 (2H, q, J=7.6 Hz), 1.39 (3H, t, J=7.1 Hz), 1.28 (3H, t, J=7.6 Hz).

### Reference Example 2

### Synthesis of ethyl 3-ethyl-1-methyl-1H-pyrazole-5-carboxylate

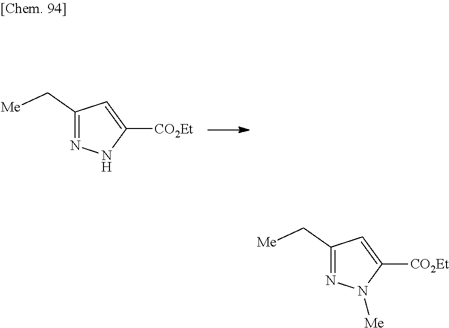 [SMILES: CCOC(=O)c1cc(CC)n[nH]1 ; CCOC(=O)c1cc(CC)nn1C]

45.0 ml of a dimethylformamide solution containing 9.32 g of ethyl 3-ethyl-1H-pyrazole-5-carboxylate and 6.31 ml of dimethyl sulfate was heated to 70° C. and stirred for 2 hours. After cooling to room temperature, water and ethyl acetate were added to the reaction mixture, and the organic layer was extracted. After separating the organic layer and the aqueous layer, the obtained organic layer was washed with saturated brine and dried with sodium sulfate. After distilling off the solvent under reduced pressure, the obtained residue was purified by silica gel column chromatography. The title compound was obtained as 6.92 g of a yellow oil.

¹H-NMR (CDCl₃) δ: 6.64 (1H, s), 4.33 (2H, q, J=7.1 Hz), 4.12 (3H, s), 2.64 (2H, q, J=7.6 Hz), 1.37 (3H, t, J=7.1 Hz), 1.24 (3H, t, J=7.6 Hz).

### Reference Example 3

### Synthesis of ethyl 4-bromo-3-ethyl-1-methyl-1H-pyrazole-5-carboxylate

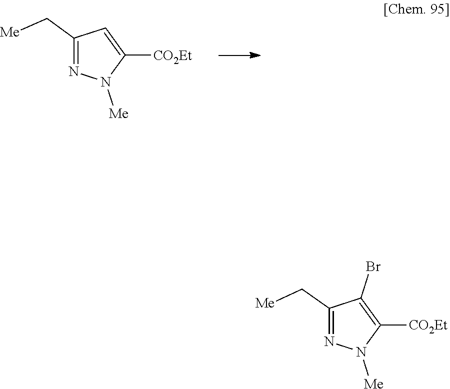 [SMILES: CCOC(=O)c1cc(CC)nn1C ; CCOC(=O)c1c(Br)c(CC)nn1C]

4.30 ml of bromine was added to 76.0 ml of a dichloromethane solution containing 7.64 g of ethyl 3-ethyl-1-methyl-1H-pyrazol-5-carboxylate cooled to 0° C. The solution was warmed to room temperature and stirred for 25 minutes. A 1% sodium thiosulfate aqueous solution and dichloromethane were added to the reaction mixture, and the organic layer was extracted. The organic layer and the aqueous layer were separated, and then the obtained organic layer was dried with sodium sulfate. After distilling off the solvent under reduced pressure, the obtained residue was purified by silica gel column chromatography. The title compound was obtained as 10.7 g of a colorless oil.

¹H-NMR (CDCl₃) δ: 4.40 (2H, q, J=7.1 Hz), 4.11 (3H, s), 2.65 (2H, q, J=7.5 Hz), 1.42 (3H, t, J=7.0 Hz), 1.24 (311, t, J=7.6 Hz).

### Reference Example 4

### Synthesis of ethyl 4-bromo-3-(1-bromoethyl)-1-methyl-1H-pyrazole-5-carboxylate

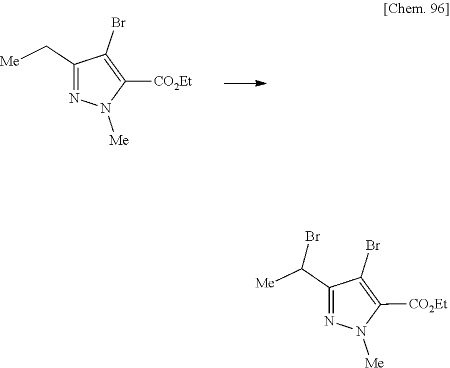

70 ml of chlorobenzene containing 7.04 g of ethyl 4-bromo-3-ethyl-1-methyl-1H-pyrazol-5-carboxylate, 5.28 g of N-bromosuccinimide, and 221 mg of azobisisobutyronitrile was stirred under heating and reflux for 1 hour and 10 minutes. After the reaction mixture was cooled to room temperature, water and dichloromethane were added, and the organic layer was extracted. The organic layer and the aqueous layer were separated, and then the obtained organic layer was dried with sodium sulfate. After the solvent was distilled off under reduced pressure, the title compound was obtained as 10.9 g of a yellow-brown oil. It was used in the next reaction without further purification.

¹H-NMR (CDCl₃) δ: 5.24 (1H, q, J=7.0 Hz), 4.41 (2H, q, J=7.1 Hz), 4.16 (3H, s), 2.11 (3H, d, J=7.0 Hz), 1.42 (3H, t, J=7.1 Hz).

### Reference Example 5

### Synthesis of ethyl 3-acetyl-4-bromo-1-methyl-1H-pyrazole-5-carboxylate

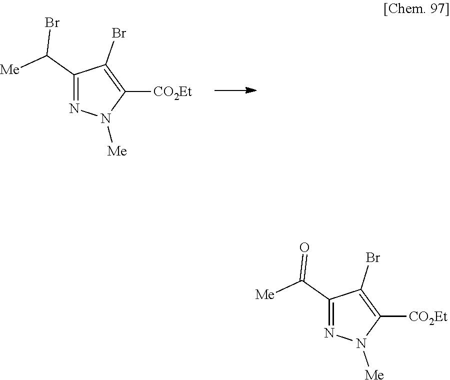 [SMILES: CCOC(=O)c1c(Br)c(C(C)Br)nn1C ; CCOC(=O)c1c(Br)c(C(C)=O)nn1C]

90 ml of acetonitrile containing 10.9 g of the ethyl 4-bromo-3-(1-bromoethyl)-1-methyl-1H-pyrazole-5-carboxylate obtained in Reference Example 4 and 9.49 g of N-methylmorpholine-N-oxide was stirred at 100° C. for 2.5 hours. After the reaction mixture was cooled to room temperature, a 1% sodium thiosulfate aqueous solution and ethyl acetate were added, and the organic layer was extracted. After separating the organic layer and the aqueous layer, the obtained organic layer was washed with saturated brine and dried with sodium sulfate. After distilling off the solvent under reduced pressure, the residue was purified by silica gel column chromatography. The title compound was obtained as 4.42 g of a light yellow solid.

¹H-NMR (CDCl₃) δ: 4.43 (2H, q, J=7.2 Hz), 4.22 (3H, s), 2.60 (3H, s), 1.44 (3H, t, J=7.2 Hz).

### Reference Example 6

### Synthesis of ethyl 4-bromo-3-(2-bromoacetyl)-1-methyl-1H-pyrazole-5-carboxylate

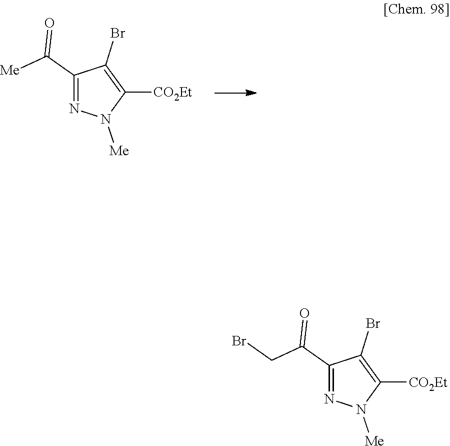 [SMILES: CCOC(=O)c1c(Br)c(C(C)=O)nn1C ; CCOC(=O)c1c(Br)c(C(=O)CBr)nn1C]

32.0 ml of tetrahydrofuran containing 3.21 g of ethyl 3-acetyl-4-bromo-1-methyl-1H-pyrazole-5-carboxylate and 4.82 g of trimethylphenylammonium tribromide was stirred at 50° C. for 20 minutes. After the reaction mixture was cooled to room temperature, water and ethyl acetate were added to the reaction mixture and then washed, and then the organic layer was extracted. After separating the organic layer and the aqueous layer, the obtained organic layer was washed with saturated brine and dried with sodium sulfate. After distilling off the solvent under reduced pressure, the residue was purified by silica gel column chromatography. The title compound was obtained as 4.41 g of a white solid.

¹H-NMR (CDCl₃) δ: 4.55 (111, s), 4.44 (1H, q, J=7.1 Hz), 4.24 (1H, s), 1.44 (2H, t, J=7.2 Hz).

### Reference Example 7

### Synthesis of ethyl 4-bromo-1-methyl-3-(7-(trifluoromethyl)imidazo[1,2-c]pyrimidin-2-yl)-1H-pyrazol-5-carboxylate

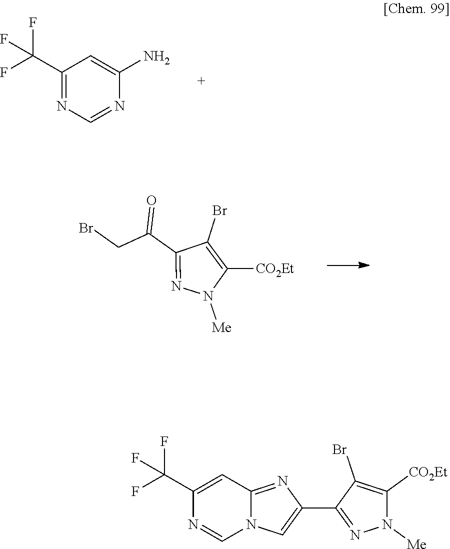 [SMILES: Nc1cc(C(F)(F)F)ncn1 ; CCOC(=O)c1c(Br)c(C(=O)CBr)nn1C ; CCOC(=O)c1c(Br)c(-c2cn3cnc(C(F)(F)F)cc3n2)nn1C]

16.6 ml of chlorobenzene containing 4.41 g of the ethyl 4-bromo-3-(2-bromoacetyl)-1-methyl-1H-pyrazole-5-carboxylate obtained in Reference Example 6 and 1.66 g of 6-(trifluoromethyl)pyrimidin-4-amine was stirred under heating and reflux for 3 hours. After the reaction mixture was cooled to room temperature, a saturated sodium bicarbonate aqueous solution and dichloromethane were added, and liquid separation was performed. The obtained organic layer was dried with sodium sulfate. After the solvent was distilled off under reduced pressure, the obtained residue was washed with diisopropyl ether to give the title compound as 3.02 g of a yellow solid.

¹H-NMR (DMSO-D₆) 6: 9.62 (1H, d, J=0.9 Hz), 8.72 (1H, s), 8.27 (1H, s), 4.39 (2H, q, J=7.1 Hz), 4.17 (311, s), 1.36 (311, t, J=7.1 Hz).

### Reference Example 8

### Synthesis of ethyl 4-(ethylthio)-1-methyl-3-(7-(trifluoromethyl)imidazo[1,2-c]pyrimidin-2-yl)-1H-pyrazole-5-carboxylate

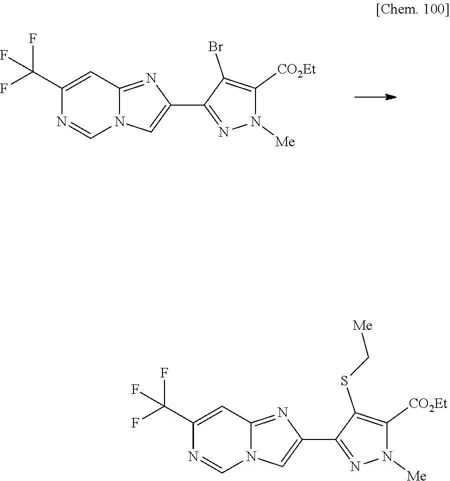 [SMILES: CCOC(=O)c1c(Br)c(-c2cn3cnc(C(F)(F)F)cc3n2)nn1C ; CCOC(=O)c1c(SCC)c(-c2cn3cnc(C(F)(F)F)cc3n2)nn1C]

27.0 ml of 1,4-dioxane containing 2.73 g of ethyl 4-bromo-1-methyl-3-(7-(trifluoromethyl)imidazo[1,2-c]pyrimidin-2-yl)-1H-pyrazole-5-carboxylate, 2.41 ml of ethanethiol, 1.20 g of tris(dibenzylideneacetone)dipalladium, 1.51 g of 4,5-bis(diphenylphosphino)-9,9-dimethylxanthene, and 5.63 ml of diisopropylethylamine was stirred at 110° C. for 9 hours. After cooling to room temperature, saturated ammonium chloride and ethyl acetate were added to the reaction mixture. After filtering the obtained residue through Celite, the Celite residue was washed with ethyl acetate. After separating the organic layer and the aqueous layer, the obtained organic layer was washed with saturated brine and dried with sodium sulfate. After distilling off the solvent under reduced pressure, the obtained residue was purified by silica gel column chromatography. The title compound was obtained as 2.50 g of a light yellow solid.

¹H-NMR (CDCl₃) δ: 9.15 (1H, d, J=0.7 Hz), 8.82 (1H, s), 7.95 (1H, d, J=0.5 Hz), 4.48 (2H, q, J=7.2 Hz), 4.27 (3H, s), 2.84 (2H, q, J=7.4 Hz), 1.47 (3H, t, J=7.2 Hz), 1.18 (3H, t, J=7.3 Hz).

### Reference Example 9

### Synthesis of 4-(ethylthio)-1-methyl-3-(7-(trifluoromethyl)imidazo[1,2-c]pyrimidin-2-yl)-1H-pyrazole-5-carboxylic acid (Compound No.: I-1)

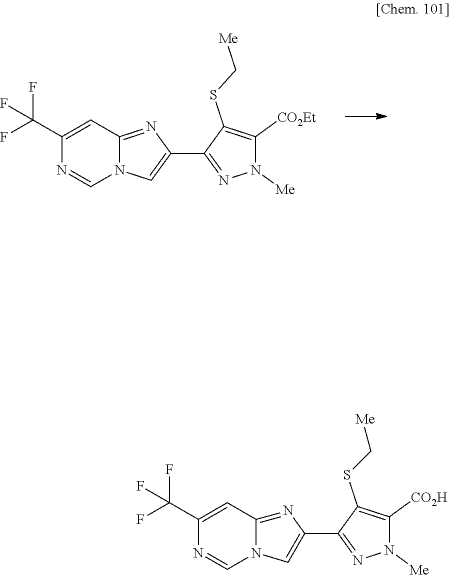 [SMILES: CCOC(=O)c1c(SCC)c(-c2cn3cnc(C(F)(F)F)cc3n2)nn1C ; CCSc1c(-c2cn3cnc(C(F)(F)F)cc3n2)nn(C)c1C(=O)O]

A mixed solution of 12.5 ml of ethanol containing 2.50 g of ethyl 4-(ethylthio)-1-methyl-3-(7-(trifluoromethyl)imidazo[1,2-c]pyrimidin-2-yl)-1H-pyrazol-5-carboxylate and 376 mg of sodium hydroxide, 12.5 ml of tetrahydrofuran, and 12.5 ml of water was stirred at room temperature for 30 minutes. 125 mg of sodium hydroxide was added to the solution, and the mixture was further stirred at room temperature for 1 hour. Water and dichloromethane were added to the reaction mixture, and liquid separation was performed. The obtained water layer was acidified with 1 N hydrochloric acid water, and then extracted by adding dichloromethane. The organic layer and the aqueous layer were separated, and then the obtained organic layer was dried with sodium sulfate. The solvent was distilled off under reduced pressure to obtain the title compound as 1.98 g of a white solid.

¹H-NMR (CDCl₃) δ: 9.17 (1H, s), 8.53 (1H, s), 7.99 (1H, s), 4.37 (3H, s), 2.95 (2H, q, J=7.4 Hz), 1.26 (3H, t, J=7.4 Hz).

### Synthesis Example 1

### Synthesis of t-butyl(4-(ethylthio)-1-methyl-3-(7-(trifluoromethyl)imidazo[1,2-c]pyrimidin-2-yl)-1H-pyrazol-5-yl)carbamate (Compound No.: 26)

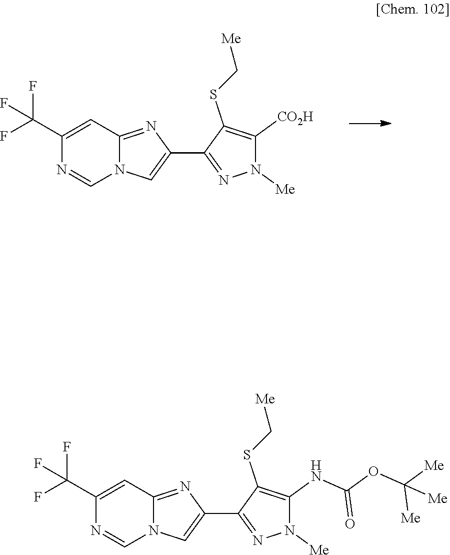 [SMILES: CCSc1c(-c2cn3cnc(C(F)(F)F)cc3n2)nn(C)c1C(=O)O ; CCSc1c(-c2cn3cnc(C(F)(F)F)cc3n2)nn(C)c1NC(=O)OC(C)(C)C]

20.0 ml of a t-butyl alcohol solution containing 1.98 g of 4-(ethylthio)-1-methyl-3-(7-(trifluoromethyl)imidazo[1,2-c]pyrimidin-2-yl)-1H-pyrazole-5-carboxylic acid, 1.50 ml of triethylamine, and 2.29 ml of diphenylphosphate azide was stirred at room temperature for 20 minutes. The solution was heated to 80° C., and the mixture was further stirred for 2.5 hours. The reaction mixture was cooled to room temperature, and then water and ethyl acetate were added thereto for extraction. The organic layer and the aqueous layer were separated, and then the obtained organic layer was dried with sodium sulfate. The solvent was distilled off under reduced pressure to obtain the title compound as 1.48 g of a transparent oil.

### Synthesis Example 2

### Synthesis of t-butyl(4-(ethylthio)-1-methyl-3-(7-(trifluoromethyl)imidazo[1,2-c]pyrimidin-2-yl)-1H-pyrazol-5-yl)(methyl)carbamate

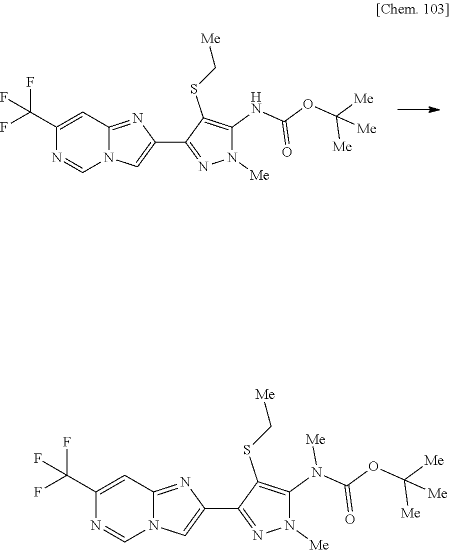 [SMILES: CCSc1c(-c2cn3cnc(C(F)(F)F)cc3n2)nn(C)c1NC(=O)OC(C)(C)C ; CCSc1c(-c2cn3cnc(C(F)(F)F)cc3n2)nn(C)c1N(C)C(=O)OC(C)(C)C]

4.60 ml of a dimethylformamide solution containing 462 mg of t-butyl(4-(ethylthio)-1-methyl-3-(7-(trifluoromethyl)imidazo[1,2-c]pyrimidin-2-yl)-1H-pyrazol-5-yl)carbamate, 325 μl of methyl iodide, and 62.6 mg of sodium hydride (oil-based, approximately 60% purity) was stirred at 0° C. for 10 minutes. The reaction mixture was heated to room temperature and then further stirred for 35 minutes. A saturated ammonium chloride aqueous solution and ethyl acetate were added to the reaction mixture, and liquid separation was performed. The obtained organic layer was washed with brine and dried with sodium sulfate. After distilling off the solvent under reduced pressure, the obtained residue was purified by silica gel column chromatography. The title compound was obtained as 295 mg of a transparent oil.

### Synthesis Example 3

### Synthesis of 4-(ethylthio)-N,1-dimethyl-3-(7-(trifluoromethyl)imidazo[1,2-c]pyrimidin-2-yl)-1H-pyrazol-5-amine (Compound No.: 29)

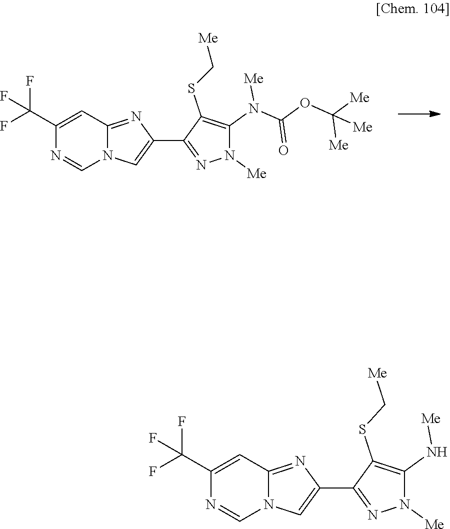 [SMILES: CCSc1c(-c2cn3cnc(C(F)(F)F)cc3n2)nn(C)c1N(C)C(=O)OC(C)(C)C ; CCSc1c(-c2cn3cnc(C(F)(F)F)cc3n2)nn(C)c1NC]

3.00 ml of a trifluoroacetic acid solution containing 287 mg of t-butyl(4-(ethylthio)-1-methyl-3-(7-(trifluoromethyl)imidazo[1,2-c]pyrimidin-2-yl)-1H-pyrazol-5-yl)(methyl)carbamate was stirred at room temperature for 20 minutes. To the reaction mixture, a saturated sodium bicarbonate aqueous solution and ethyl acetate were added, and liquid separation was performed. The obtained organic layer was washed with brine and dried with sodium sulfate. After distilling off the solvent under reduced pressure, the obtained residue was purified by silica gel column chromatography. The title compound was obtained as 105 mg of a transparent oil.

### Synthesis Example 4

### Synthesis of N-(4-(ethylthio)-1-methyl-3-(7-(trifluoromethyl)imidazo[1,2-c]pyrimidin-2-yl)-1H-pyrazol-5-yl)-2,2,2-trifluoro-N-methylacetamide (Compound No.: 30)

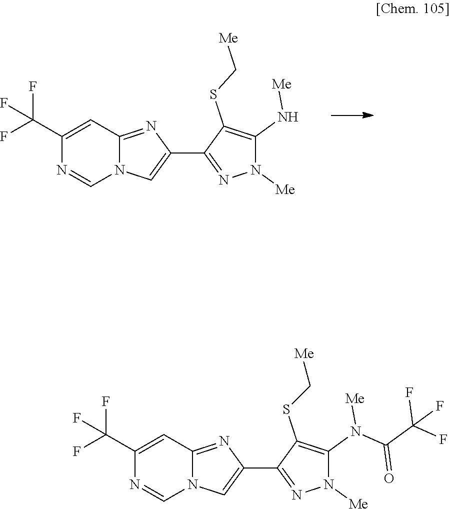 [SMILES: CCSc1c(-c2cn3cnc(C(F)(F)F)cc3n2)nn(C)c1NC ; CCSc1c(-c2cn3cnc(C(F)(F)F)cc3n2)nn(C)c1N(C)C(=O)C(F)(F)F]

3.00 ml of dichloromethane containing 96.7 mg of 4-(ethylthio)-N,1-dimethyl-3-(7-(trifluoromethyl)imidazo[1,2-c]pyrimidin-2-yl)-1H-pyrazol-5-amine, 114 μl of trifluoroacetic anhydride, and 114 μl of triethylamine was stirred at 0° C. for 30 minutes. To the reaction mixture, a saturated sodium bicarbonate aqueous solution and ethyl acetate were added, and liquid separation was performed. The obtained organic layer was washed with brine and dried with sodium sulfate. After distilling off the solvent under reduced pressure, the obtained residue was purified by silica gel column chromatography. The title compound was obtained as 115 mg of a white solid.

### Synthesis Example 5

### Synthesis of 4-(ethylthio)-1-methyl-3-(7-(trifluoromethyl)imidazo[1,2-c]pyrimidin-2-yl)-1H-pyrazol-5-amine (Compound No.: 39)

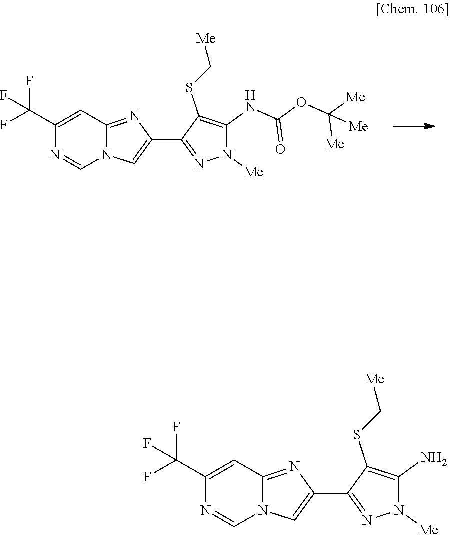 [SMILES: CCSc1c(-c2cn3cnc(C(F)(F)F)cc3n2)nn(C)c1NC(=O)OC(C)(C)C ; CCSc1c(-c2cn3cnc(C(F)(F)F)cc3n2)nn(C)c1N]

8.00 ml of a dichloromethane solution containing 880 mg of t-butyl(4-(ethylthio)-1-methyl-3-(7-(trifluoromethyl)imidazo[1,2-c]pyrimidin-2-yl)-1H-pyrazol-5-yl)carbamate and 8.00 ml of trifluoroacetic acid was stirred at room temperature for 3 hours. To the reaction mixture, a saturated sodium bicarbonate aqueous solution and dichloromethane were added, and liquid separation was performed. The obtained organic layer was washed with brine and dried with sodium sulfate. The solvent was distilled off under reduced pressure to obtain the title compound as 643 mg of a gray solid.

### Synthesis Example 6

### Synthesis of 4-(ethylsulfonyl)-1-methyl-3-(7-(trifluoromethyl)imidazo[1,2-c]pyrimidin-2-yl)-1H-pyrazol-5-amine (Compound No.: 100)

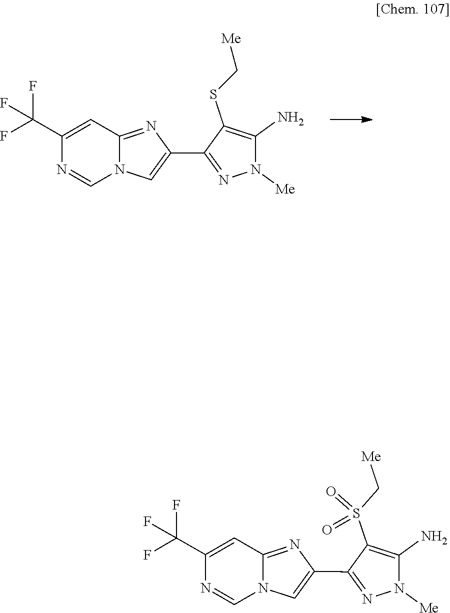 [SMILES: CCSc1c(-c2cn3cnc(C(F)(F)F)cc3n2)nn(C)c1N ; CCS(=O)(=O)c1c(-c2cn3cnc(C(F)(F)F)cc3n2)nn(C)c1N]

7.00 ml of a dichloromethane solution containing 40.0 mg of 4-(ethylthio)-1-methyl-3-(7-(trifluoromethyl)imidazo[1,2-c]pyrimidin-2-yl)-1H-pyrazol-5-amine and 57.6 mg of 3-chloroperbenzoic acid (ca. 70% purity) was stirred at room temperature for 4 hours. Water and ethyl acetate were added to the reaction mixture, and liquid separation was performed. The obtained organic layer was washed with a saturated sodium bicarbonate aqueous solution, and then dried with sodium sulfate. After distilling off the solvent under reduced pressure, the obtained residue was purified by silica gel column chromatography. The title compound was obtained as 29.5 mg of a yellow solid.

### Synthesis Example 7

### Synthesis of N-(4-(ethylthio)-1-methyl-3-(7-(trifluoromethyl)imidazo[1,2-c]pyridin-2-yl)-1H-pyrazol-5-yl)-2,2,2-trifluoroacetamide (Compound No.: 27)

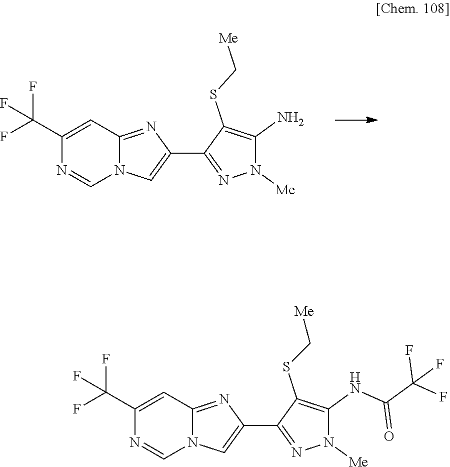 [SMILES: CCSc1c(-c2cn3cnc(C(F)(F)F)cc3n2)nn(C)c1N ; CCSc1c(-c2cn3cnc(C(F)(F)F)cc3n2)nn(C)c1NC(=O)C(F)(F)F]

1.50 ml of dichloromethane containing 150 mg of 4-(ethylthio)-1-methyl-3-(7-(trifluoromethyl)imidazo[1,2-c]pyrimidin-2-yl)-1H-pyrazol-5-amine, 183 μl of trifluoroacetic anhydride, and 184 μl of triethylamine was stirred at room temperature for 2 hours. To the reaction mixture, a saturated sodium bicarbonate aqueous solution and ethyl acetate were added, and liquid separation was performed. The obtained organic layer was washed with brine and dried with sodium sulfate. After distilling off the solvent under reduced pressure, the obtained residue was purified by silica gel column chromatography. The title compound was obtained as 184.5 mg of a white solid.

### Synthesis Example 8

### Synthesis of N-(4-(ethylsulfonyl)-1-methyl-3-(7-(trifluoromethyl)imidazo[1,2-c]pyrimidin-2-yl)-1H-pyrazol-5-yl)-2,2,2-trifluoroacetamide (Compound No.: 1)

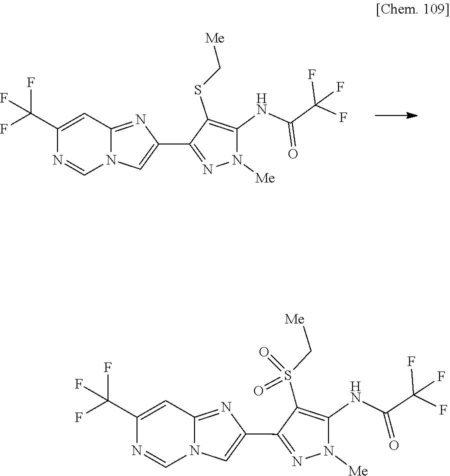 [SMILES: CCSc1c(-c2cn3cnc(C(F)(F)F)cc3n2)nn(C)c1NC(=O)C(F)(F)F ; CCS(=O)(=O)c1c(-c2cn3cnc(C(F)(F)F)cc3n2)nn(C)c1NC(=O)C(F)(F)F]

1 ml of a dichloromethane solution containing 100 mg of N-(4-(ethylthio)-1-methyl-3-(7-(trifluoromethyl)imidazo[1,2-c]pyridin-2-yl)-1H-pyrazol-5-yl)-2,2,2-trifluoroacetamide and 135 mg of 3-chloroperbenzoic acid (ca. 70% purity) was stirred at room temperature for 3 hours. To the reaction mixture, a sodium thiosulfate aqueous solution and ethyl acetate were added, and liquid separation was performed. The obtained organic layer was washed with a saturated sodium bicarbonate aqueous solution, and then dried with sodium sulfate. After distilling off the solvent under reduced pressure, the obtained residue was purified by silica gel column chromatography. The title compound was obtained as 68.1 mg of a white solid.

### Synthesis Example 9

### Synthesis of N-(3-(3-chloro-7-(trifluoromethyl)imidazo[1,2-c]pyrimidin-2-yl)-4-(ethylsulfonyl)-1-methyl-1H-pyrazol-5-yl)-2,2,2-trifluoroacetamide (Compound No.: 53)

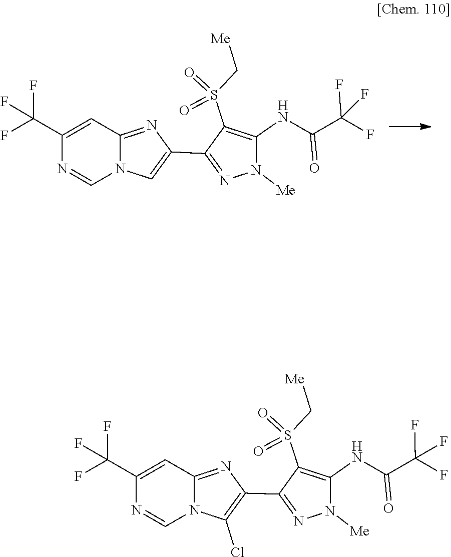 [SMILES: CCS(=O)(=O)c1c(-c2cn3cnc(C(F)(F)F)cc3n2)nn(C)c1NC(=O)C(F)(F)F ; CCS(=O)(=O)c1c(-c2nc3cc(C(F)(F)F)ncn3c2Cl)nn(C)c1NC(=O)C(F)(F)F]

1 ml of an acetonitrile solution containing 27.0 mg of N-(4-(ethylsulfonyl)-1-methyl-3-(7-(trifluoromethyl)imidazo[1,2-c]pyrimidin-2-yl)-1H-pyrazol-5-yl)-2,2,2-trifluoroacetamide, 10.0 mg of N-chlorosuccinimide, and chlorotrimethylsilane was stirred at room temperature for 30 minutes. To the reaction mixture, a sodium thiosulfate aqueous solution and ethyl acetate were added, and liquid separation was performed. The obtained organic layer was dried with sodium sulfate. After distilling off the solvent under reduced pressure, the residue was purified by silica gel column chromatography. The title compound was obtained as 9.6 mg of a white solid.

### Synthesis Example 10

### Synthesis of N-(3-(3-bromo-7-(trifluoromethyl)imidazo[1,2-c]pyrimidin-2-yl)-4-(ethylsulfonyl)-1-methyl-1H-pyrazol-5-yl)-2,2,2-trifluoroacetamide (Compound No.: 54)

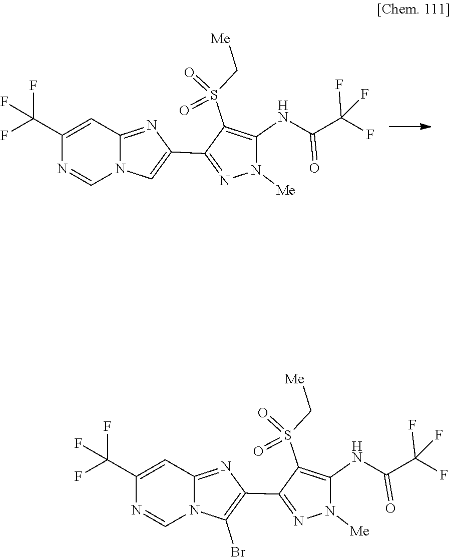 [SMILES: CCS(=O)(=O)c1c(-c2cn3cnc(C(F)(F)F)cc3n2)nn(C)c1NC(=O)C(F)(F)F ; CCS(=O)(=O)c1c(-c2nc3cc(C(F)(F)F)ncn3c2Br)nn(C)c1NC(=O)C(F)(F)F]

2 ml of a dimethylformamide solution containing 40.0 mg of N-(4-(ethylsulfonyl)-1-methyl-3-(7-(trifluoromethyl)imidazo[1,2-c]pyrimidin-2-yl)-1H-pyrazol-5-yl)-2,2,2-trifluoroacetamide and 19.8 mg of N-bromosuccinimide was stirred at room temperature for 1.5 hours. To the reaction mixture, a sodium thiosulfate aqueous solution and ethyl acetate were added, and liquid separation was performed. The obtained organic layer was dried with sodium sulfate. After distilling off the solvent under reduced pressure, the residue was purified by silica gel column chromatography. The title compound was obtained as 34.8 mg of a white solid.

### Synthesis Example 11

### Synthesis of N-(4-(ethylthio)-1-methyl-3-(7-(trifluoromethyl)imidazo[1,2-c]pyrimidin-2-yl)-1H-pyrazol-5-yl)picolinamide (Compound No.: 84)

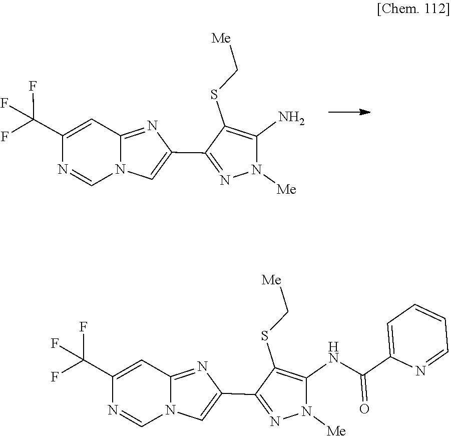 [SMILES: CCSc1c(-c2cn3cnc(C(F)(F)F)cc3n2)nn(C)c1N ; CCSc1c(-c2cn3cnc(C(F)(F)F)cc3n2)nn(C)c1NC(=O)c1ccccn1]

1.00 ml of a dichloroethane solution containing 60.0 mg of 4-(ethylthio)-1-methyl-3-(7-(trifluoromethyl)imidazo[1,2-c]pyrimidin-2-yl)-1H-pyrazol-5-amine, 93.5 mg of pyridine-2-carbonyl chloride hydrochloride, and 84.8 μl of pyridine was stirred at room temperature for 2 hours. To the reaction mixture, a saturated sodium bicarbonate aqueous solution and ethyl acetate were added, and liquid separation was performed. The obtained organic layer was dried with sodium sulfate. After distilling off the solvent under reduced pressure, the residue was purified by silica gel column chromatography. The title compound was obtained as 41.5 mg of a white solid.

### Synthesis Example 12

### Synthesis of N-(4-(ethylsulfonyl)-1-methyl-3-(7-(trifluoromethyl)imidazo[1,2-c]pyrimidin-2-yl)-1H-pyrazol-5-yl)picolinamide (Compound No.: 86)

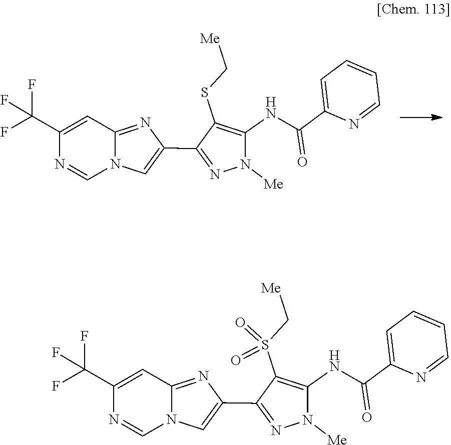 [SMILES: CCSc1c(-c2cn3cnc(C(F)(F)F)cc3n2)nn(C)c1NC(=O)c1ccccn1 ; CCS(=O)(=O)c1c(-c2cn3cnc(C(F)(F)F)cc3n2)nn(C)c1NC(=O)c1ccccn1]

1 ml of a dichloromethane solution containing 31.2 mg of N-(4-(ethylthio)-1-methyl-3-(7-(trifluoromethyl)imidazo[1,2-c]pyrimidin-2-yl)-1H-pyrazol-5-yl)picolinamide and 41.2 mg of 3-chloroperbenzoic acid (ca. 70% purity) was stirred at room temperature for 2 hours. To the reaction mixture, a 3% sodium thiosulfate aqueous solution, a saturated sodium bicarbonate aqueous solution, and dichloromethane were added, and liquid separation was performed. The obtained organic layer was dried with magnesium sulfate. After distilling off the solvent under reduced pressure, the obtained residue was purified by silica gel column chromatography. The title compound was obtained as 27.9 mg of a white solid.

### Synthesis Example 10

### Ethyl 5-amino-1-methyl-1H-pyrazole-3-carboxylate

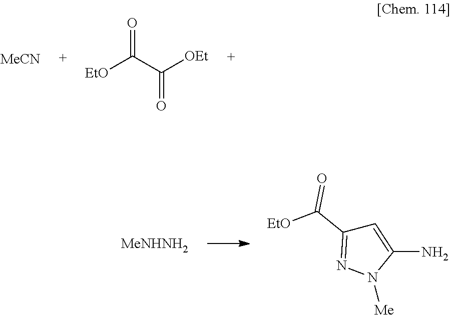 [SMILES: "MeCN" ; "MeNHNH2" ; CCOC(=O)C(=O)OCC ; CCOC(=O)c1cc(N)n(C)n1]

To 60.0 ml of an acetonitrile solution containing 15.0 g of diethyl oxalate, 11.6 g of t-butoxy potassium was added at 0° C. The reaction mixture was heated to room temperature and then stirred for 30 minutes. After filtering the obtained reaction mixture, the residue was washed with acetonitrile to obtain 10.6 g of 1-cyano-3-ethoxy-3-oxopro-1-pen-2-noate as a white solid. 3.72 ml of methylhydrazine, 13.6 ml of trifluoroacetic acid, and 100 ml of ethanol were added to the solid, and the mixture was stirred at 80° C. for 2.5 hours. After the reaction mixture was cooled to room temperature, a saturated sodium bicarbonate aqueous solution and ethyl acetate were added, and liquid separation was performed. The obtained organic layer was dried with sodium sulfate. After distilling off the solvent under reduced pressure, the residue was purified by silica gel column chromatography. The title compound was obtained as 6.98 g of a brown oil.

¹H-NMR (CDCl₃) δ: 6.09 (1H, s), 4.37 (2H, q, J=7.2 Hz), 3.75 (3H, s), 1.38 (3H, t, J=7.2 Hz).

### Reference Example 11

### Ethyl 5-(bis(t-butoxycarbonyl)amino)-1-methyl-1H-pyrazole-3-carboxylate

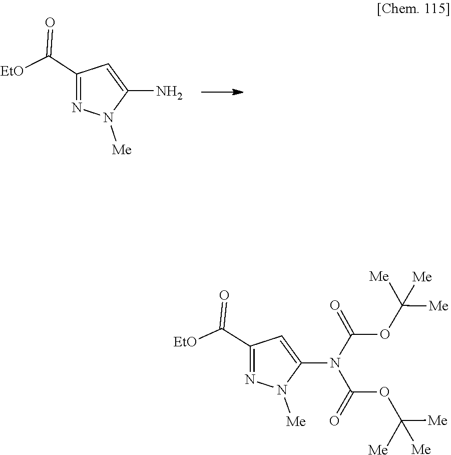 [SMILES: CCOC(=O)c1cc(N)n(C)n1 ; CCOC(=O)c1cc(N(C(=O)OC(C)(C)C)C(=O)OC(C)(C)C)n(C)n1]

70.0 ml of a tetrahydrofuran solution containing 6.98 g of ethyl 5-amino-1-methyl-1H-pyrazole-3-carboxylate, 23.4 ml of dii-t-butyl dicarbonate, and 1.01 g of dimethylaminopyridine was stirred at room temperature for 15 minutes. Ethyl acetate and an ammonium chloride aqueous solution were added to the reaction mixture, and liquid separation was performed. The obtained organic layer was washed with brine and dried with sodium sulfate. After the solvent was distilled off under reduced pressure, the title compound was obtained as 21.0 g of a brown oil. It was used in the next reaction without further purification.

¹H-NMR (CDCl₃) δ: 6.66 (1H, s), 4.43-4.38 (2H, m), 3.75 (3H, s), 1.42-1.40 (21H, m).

### Reference Example 12

### Ethyl 5-((t-butoxycarbonyl)amino)-1-methyl-1H-pyrazole-3-carboxylate

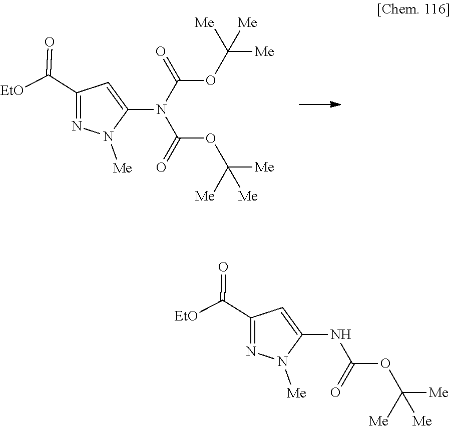 [SMILES: CCOC(=O)c1cc(N(C(=O)OC(C)(C)C)C(=O)OC(C)(C)C)n(C)n1 ; CCOC(=O)c1cc(NC(=O)OC(C)(C)C)n(C)n1]

150 ml of an acetonitrile solution containing 21.0 g of ethyl 5-(bis(t-butoxycarbonyl)amino)-1-methyl-1H-pyrazole-3-carboxylate and 5.38 g of lithium bromide was heated and refluxed for 40 minutes. After the reaction mixture was cooled to room temperature, a saturated sodium bicarbonate aqueous solution and ethyl acetate were added, and liquid separation was performed. Ethyl acetate and an ammonium chloride aqueous solution were added to the reaction mixture, and liquid separation was performed. The obtained organic layer was washed with brine and dried with sodium sulfate. After distilling off the solvent under reduced pressure, the residue was purified by silica gel column chromatography. The title compound was obtained as 7.34 g of a brown oil.

¹H-NMR (CDCl₃) δ: 6.69 (111, s), 6.18 (111, br s), 4.39 (211, q, J=7.2 Hz), 3.83 (3H, s), 1.51 (9H, s), 1.38 (3H, t, J=7.2 Hz).

### Reference Example 13

### Ethyl 5-((t-butoxycarbonyl)amino)-4-iodo-1-methyl-1H-pyrazole-3-carboxylate

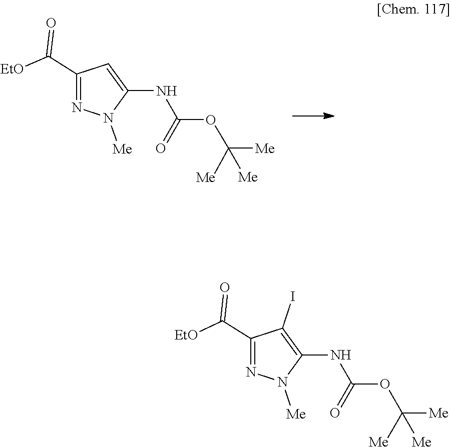 [SMILES: CCOC(=O)c1cc(NC(=O)OC(C)(C)C)n(C)n1 ; CCOC(=O)c1nn(C)c(NC(=O)OC(C)(C)C)c1I]

37.0 ml of dimethylformamide containing 7.34 g of ethyl 5-((t-butoxycarbonyl)amino)-1-methyl-1H-pyrazole-3-carboxylate and 9.20 g of N-Iodosuccinimide was stirred at 50° C. for 1.5 hours. After cooling to room temperature, a 1% sodium thiosulfate aqueous solution and ethyl acetate were added to the reaction mixture, and liquid separation was performed. The obtained organic layer was washed with saturated brine and dried with sodium sulfate. After distilling off the solvent under reduced pressure, the obtained residue was purified by silica gel column chromatography. The title compound was obtained as 9.84 g of a brown oil.

¹H-NMR (CDCl₃) δ: 5.97 (1H, br s), 4.43 (2H, q, J=7.1 Hz), 3.92 (3H, s), 1.50 (9H, s), 1.42 (3H, t, J=7.1 Hz).

### Reference Example 14

### Synthesis of ethyl 5-((t-butoxycarbonyl)amino)-4-(ethylthio)-1-methyl-1H-pyrazole-3-carboxylate

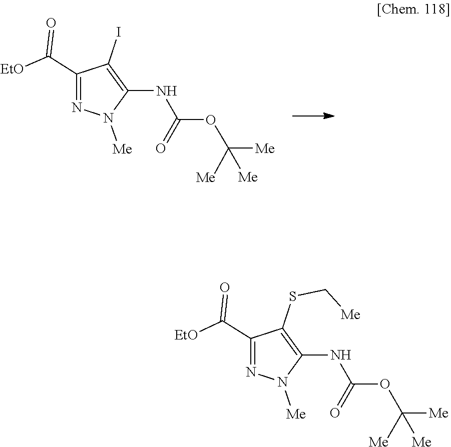 [SMILES: CCOC(=O)c1nn(C)c(NC(=O)OC(C)(C)C)c1I ; CCOC(=O)c1nn(C)c(NC(=O)OC(C)(C)C)c1SCC]

98.0 ml of 1,4-dioxane containing 9.84 g of ethyl 5-((t-butoxycarbonyl)amino)-4-iodo-1-methyl-1H-pyrazole-3-carboxylate, 5.52 ml of ethanethiol, 1.14 g of tris(dibenzylideneacetone)dipalladium, 1.44 g of 4,5-bis(diphenylphosphino)-9,9-dimethylxanthene, and 12.9 ml of diisopropylethylamine was stirred at 110° C. for 1.5 hours. After cooling to room temperature, saturated ammonium chloride and ethyl acetate were added to the reaction mixture. After filtering the obtained residue through Celite, the Celite residue was washed with ethyl acetate. After separating the organic layer and the aqueous layer, the obtained organic layer was washed with saturated brine and dried with sodium sulfate. After distilling off the solvent under reduced pressure, the obtained residue was purified by silica gel column chromatography. The title compound was obtained as 7.38 g of an orange oil.

¹H-NMR (CDCl₃) δ: 6.30 (111, br s), 4.43 (211, q, J=7.1 Hz), 3.88 (3H, s), 2.77 (2H, q, J=7.3 Hz), 1.50 (9H, s), 1.42 (3H, t, J=7.1 Hz), 1.14 (3H, t, J=7.3 Hz).

### Reference Example 15

### Synthesis of 5-((t-butoxycarbonyl)amino)-4-(ethylthio)-1-methyl-1H-pyrazole-3-carboxylic acid

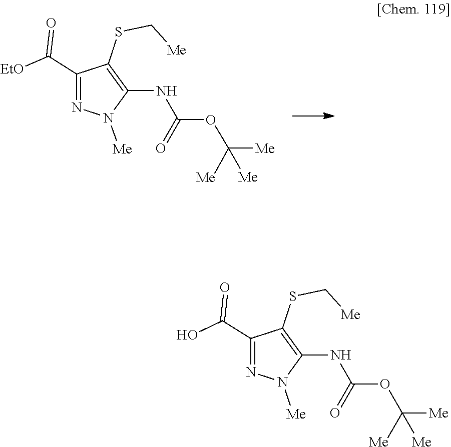 [SMILES: CCOC(=O)c1nn(C)c(NC(=O)OC(C)(C)C)c1SCC ; CCSc1c(C(=O)O)nn(C)c1NC(=O)OC(C)(C)C]

30.0 ml of ethanol containing 7.38 g of ethyl 5-((t-butoxycarbonyl)amino)-4-(ethylthio)-1-methyl-1H-pyrazole-3-carboxylate and 1.79 g of sodium hydroxide, and a mixed solvent of 30.0 ml of tetrahydrofuran and 30.0 g of water were stirred at 50° C. for 30 minutes. 1 N aqueous hydrochloric acid and dichloromethane were added to the reaction mixture, and the organic layer was separated. The obtained organic layer was dried with sodium sulfate. The solvent was distilled off under reduced pressure to obtain the title compound as 6.67 g of a brown solid.

¹H-NMR (CDCl₃) δ: 6.40 (111, s), 3.89 (3H, s), 2.77 (2H, q, J=7.3 Hz), 1.51 (9H, s), 1.16 (3H, t, J=7.3 Hz).

### Reference Example 16

### Synthesis of 1,6-diamino-4-(trifluoromethyl)pyrimidin-1-ium-2,4,6-trimethylbenzenesulfonate

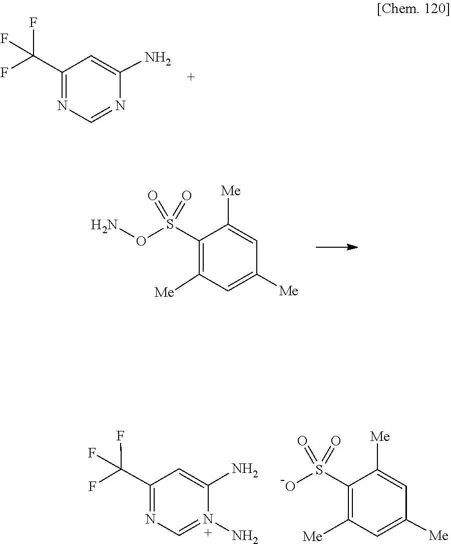

To 10.0 ml of a dichloromethane solution containing 1.37 g of O-(mesitylsulfonyl)hydroxylamine, 10.0 ml of a dichloromethane solution containing 1.03 g of 6-(trifluoromethyl)pyrimidin-4-amine was added dropwise at 0° C. The reaction mixture was heated to room temperature and then stirred for 2.5 hours. The obtained reaction mixture was filtered, and then the residue was washed with dichloromethane. The title compound was obtained as 2.29 g of a white solid.

¹H-NMR (DMSO-D₆) 8: 10.03 (1H, s), 9.53 (1H, s), 8.92 (1H, s), 7.46 (1H, s), 6.81 (211, s), 6.75 (2H, s), 2.49 (6H, s), 2.17 (3H, s).

### Synthesis Example 13

### Synthesis of t-butyl (4-(ethylthio)-1-methyl-3-(7-(trifluoromethyl)-[1,2,4]triazolo[1,5-c]pyrimidin-2-yl)-1H-pyrazol-5-yl)carbamate (Compound No.: 4)

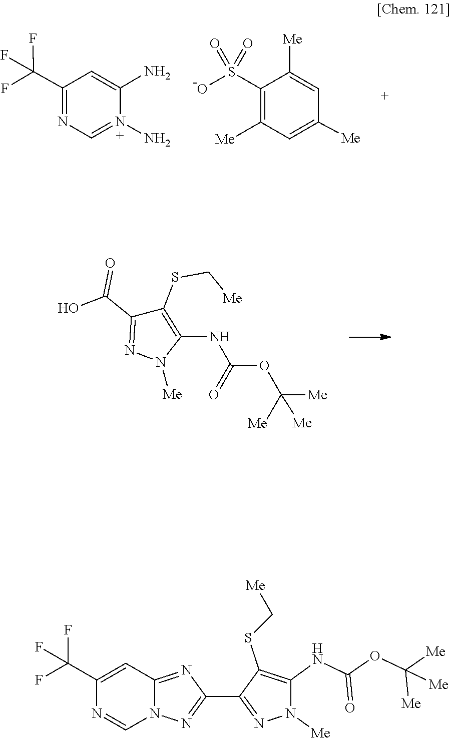

12.0 ml of pyridine containing 1.21 g of 1,6-diamino-4-(trifluoromethyl)pyrimidin-1-ium-2,4,6-trimethylbenzenesulfonate, 1.86 g of 5-((t-butoxycarbonyl)amino)-4-(ethylthio)-1-methyl-1H-pyrazole-3-carboxylic acid, 797 mg of 1-(3-dimethylaminopropyl)-3-ethylcarbodiimide hydrochloride was stirred at room temperature for 10 minutes. The reaction mixture was heated to 120° C. and then further stirred for 2 hours. After cooling to room temperature, a saturated sodium bicarbonate aqueous solution and ethyl acetate were added, and liquid separation was performed. The obtained organic layer was washed with saturated brine and dried with sodium sulfate. After distilling off the solvent under reduced pressure, the obtained residue was purified by silica gel column chromatography. The title compound was obtained as 221 mg of a white solid.

### Synthesis Example 14

### Synthesis of 4-(ethylthio)-1-methyl-3-(7-(trifluoromethyl)-[1,2,4]triazolo[1,5-c]pyrimidin-2-yl)-1H-pyrazol-5-amine (Compound No.: 5)

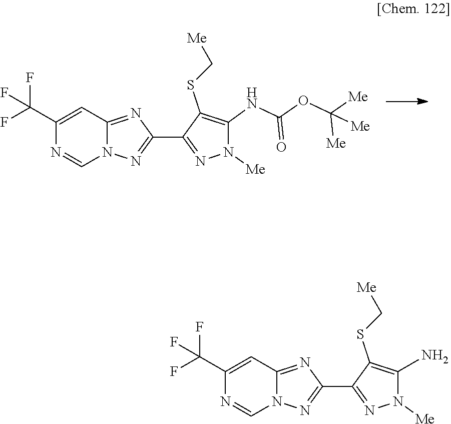 [SMILES: CCSc1c(-c2nc3cc(C(F)(F)F)ncn3n2)nn(C)c1NC(=O)OC(C)(C)C ; CCSc1c(-c2nc3cc(C(F)(F)F)ncn3n2)nn(C)c1N]

3.00 ml of trifluoroacetic acid containing 221 mg of t-butyl (4-(ethylthio)-1-methyl-3-(7-(trifluoromethyl)-[1,2,4]triazolo[1,5-c]pyrimidin-2-yl)-1H-pyrazol-5-yl)carbamate was stirred at room temperature for 45 minutes. After adding toluene to the reaction mixture, the solvent was distilled off under reduced pressure to obtain the title compound as 225 mg of a brown oil. It was used in the next reaction without further purification.

### Synthesis Example 15

### Synthesis of N-(4-(ethylthio)-1-methyl-3-(7-(trifluoromethyl)-[1,2,4]triazolo[1,5-c]pyrimidin-2-yl)-1H-pyrazol-5-yl)acetamide (Compound No.: 10)

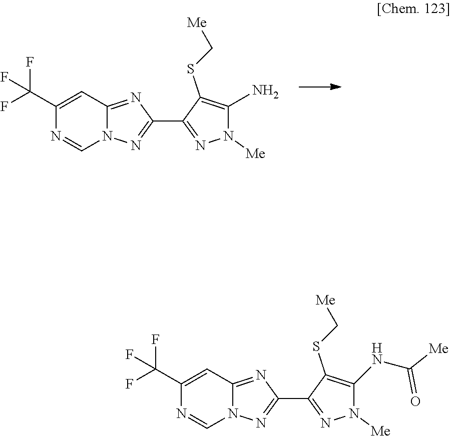 [SMILES: CCSc1c(-c2nc3cc(C(F)(F)F)ncn3n2)nn(C)c1N ; CCSc1c(-c2nc3cc(C(F)(F)F)ncn3n2)nn(C)c1NC(C)=O]

177 μl of acetyl chloride was added to 3.00 ml of dichloromethane containing 225 mg of 4-(ethylthio)-1-methyl-3-(7-(trifluoromethyl)-[1,2,4]triazolo[1,5-c]pyrimidin-2-yl)-1H-pyrazol-5-amine and 201 μl of pyridine at 0° C. The reaction mixture was heated to room temperature and then stirred for 1 hour.

To the reaction mixture, a saturated sodium bicarbonate aqueous solution and ethyl acetate were added, and the organic layer was separated. The obtained organic layer was washed with saturated brine and dried with sodium sulfate. After distilling off the solvent under reduced pressure, the obtained residue was purified by silica gel column chromatography. The title compound was obtained as 135 mg of a white solid.

### Synthesis Example 16

### Synthesis of N-(4-(Ethylsulfonyl)-1-methyl-3-(7-(trifluoromethyl)-[1,2,4]triazolo[1,5-c]pyrimidin-2-yl)-1H-pyrazol-5-yl)acetamide (Compound No.: 11)

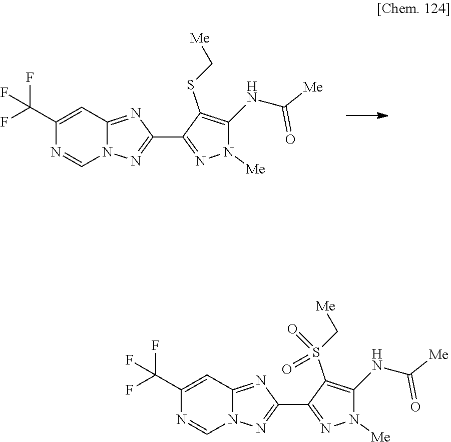 [SMILES: CCSc1c(-c2nc3cc(C(F)(F)F)ncn3n2)nn(C)c1NC(C)=O ; CCS(=O)(=O)c1c(-c2nc3cc(C(F)(F)F)ncn3n2)nn(C)c1NC(C)=O]

3.00 ml of a dichloromethane solution containing 127 mg of N-(4-(ethylthio)-1-methyl-3-(7-(trifluoromethyl)-[1,2,4]triazolo[1,5-c]pyrimidin-2-yl)-1H-pyrazol-5-yl)acetamide and 195 mg of 3-chloroperbenzoic acid (ca. 70% purity) was stirred at 0° C. for 30 minutes. To the reaction mixture, a 1% sodium thiosulfate aqueous solution, a saturated sodium bicarbonate aqueous solution, and ethyl acetate were added, and liquid separation was performed. The obtained organic layer was washed with saturated brine and dried with sodium sulfate. After the solvent was distilled off under reduced pressure, the obtained residue was washed with ethanol. The title compound was obtained as 99.8 mg of a white solid.

Table 7 shows the compounds synthesized according to the Examples described above, but the present invention is not limited thereto.

In Table 7, Ph represents a phenyl group, 2-Py represents a pyridin-2-yl group, 3-Py represents a pyridin-3-yl group, 4-Py represents a pyridin-4-yl group, 1-Pyra represents a 1H-pyrazol-1-yl group, and 1-Tria represents a 1H-triazol-1-yl group.

In Table 7, the structure A is as follows.

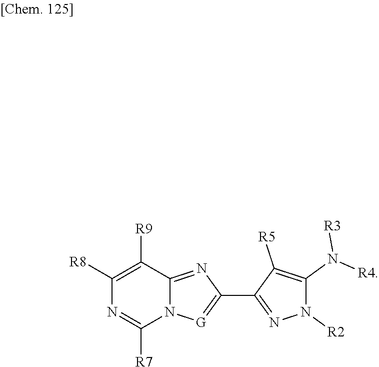

In Table 7, the structure B is as follows.

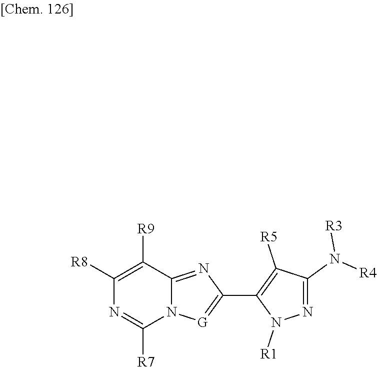

TABLE 7-1
Table 7

| Compound | | | | | | | | | | |
|---|---|---|---|---|---|---|---|---|---|---|
| No. | Structure | R1 | R2 | R3 | R4 | R5 | G | R7 | R8 | R9 |
| 1 | A | — | Me | H | —C(═O)CF₃ | SO₂Et | C—H | H | CF₃ | H |
| 2 | A | — | Me | H | —C(═O)CF₂H | SO₂Et | C—H | H | CF₃ | H |
| 3 | A | — | Me | H | —C(═O)Me | SO₂Et | C—H | H | CF₃ | H |
| 4 | A | — | Me | H | —C(═O)O—t-Bu | SEt | N | H | CF₃ | H |
| 5 | A | — | Me | H | H | SEt | N | H | CF₃ | H |
| 6 | A | — | Me | H | —C(═O)CF₃ | SEt | N | H | CF₃ | H |
| 7 | A | — | Me | H | —C(═O)CF₂H | SEt | N | H | CF₃ | H |
| 8 | A | — | Me | H | —C(═O)CF₃ | SO₂Et | N | H | CF₃ | H |
| 9 | A | — | Me | H | —C(═O)CF₂H | SO₂Et | N | H | CF₃ | H |
| 10 | A | — | Me | H | —C(═O)Me | SEt | N | H | CF₃ | H |
| 11 | A | — | Me | H | —C(═O)Me | SO₂Et | N | H | CF₃ | H |
| 12 | A | — | Me | Me | —C(═O)Me | SO₂Et | N | H | CF₃ | H |
| 13 | A | — | Me | H | —C(═O)OMe | SEt | N | H | CF₃ | H |
| 14 | A | — | Me | H | —C(═O)OMe | SO₂Et | N | H | CF₃ | H |
| 15 | A | — | Me | H | —C(═O)CF₂CF₃ | SEt | C—H | H | CF₃ | H |
| 16 | A | — | Me | H | —C(═O)CF₂CF₂CF₃ | SEt | C—H | H | CF₃ | H |
| 17 | A | — | Me | H | —C(═O)CF₂CF₃ | SO₂Et | C—H | H | CF₃ | H |
| 18 | A | — | Me | H | —C(═O)CF₂CF₂CF₃ | SO₂Et | C—H | H | CF₃ | H |
| 19 | A | — | Me | H | —C(═O)CF₂CF₃ | SEt | N | H | CF₃ | H |
| 20 | A | — | Me | H | —C(═O)CF₂CF₂CF₃ | SEt | N | H | CF₃ | H |
| 21 | A | — | Me | H | —C(═O)Et | SEt | N | H | CF₃ | H |
| 22 | A | — | Me | H | —C(═O)Pr | SEt | N | H | CF₃ | H |
| 23 | A | — | Me | H | —C(═O)CF₂CF₃ | SO₂Et | N | H | CF₃ | H |
| 24 | A | — | Me | H | —C(═O)CF₂CF₂CF₃ | SO₂Et | N | H | CF₃ | H |
| 25 | A | — | Me | H | —C(═O)Pr | SO₂Et | N | H | CF₃ | H |
| 26 | A | — | Me | H | —C(═O)O—t-Bu | SEt | C—H | H | CF₃ | H |
| 27 | A | — | Me | H | —C(═O)CF₃ | SEt | C—H | H | CF₃ | H |
| 28 | A | — | Me | Me | —C(═O)O—t-Bu | SEt | C—H | H | CF₃ | H |
| 29 | A | — | Me | H | Me | SEt | C—H | H | CF₃ | H |
| 30 | A | — | Me | Me | —C(═O)CF₃ | SEt | C—H | H | CF₃ | H |
| 31 | A | — | Me | H | Me | SO₂Et | C—H | H | CF₃ | H |
| 32 | A | — | Me | Me | —C(═O)CF₃ | SO₂Et | C—H | H | CF₃ | H |
| 33 | A | — | Me | Me | —C(═O)CF₂CF₃ | SEt | C—H | H | CF₃ | H |
| 34 | A | — | Me | Me | —C(═O)CF₂CF₃ | SO₂Et | C—H | H | CF₃ | H |
| 35 | A | — | Me | Me | —C(═O)CF₂CF₂CF₃ | SEt | C—H | H | CF₃ | H |
| 36 | A | — | Me | Me | —C(═O)CF₂CF₂CF₃ | SO₂Et | C—H | H | CF₃ | H |
| 37 | A | — | Me | Me | —C(═O)CF₂H | SEt | C—H | H | CF₃ | H |
| 38 | A | — | Me | Me | —C(═O)CF₂H | SO₂Et | C—H | H | CF₃ | H |
| 39 | A | — | Me | H | H | SEt | C—H | H | CF₃ | H |
| 40 | A | — | Me | H | —C(═O)OMe | SEt | C—H | H | CF₃ | H |
| 41 | A | — | Me | H | —C(═O)Et | SEt | C—H | H | CF₃ | H |
| 42 | A | — | Me | H | —C(═O)Et | SO₂Et | C—H | H | CF₃ | H |
| 43 | A | — | Me | H | —C(═O)OMe | SO₂Et | C—H | H | CF₃ | H |
| 44 | A | — | Me | H | —C(═O)OEt | SO₂Et | C—H | H | CF₃ | H |
| 45 | A | — | Me | H | —C(═O)OPr | SO₂Et | C—H | H | CF₃ | H |
| 46 | A | — | Me | H | —C(═O)CF₂CF₂CF₂CF₃ | SEt | N | H | CF₃ | H |
| 47 | A | — | Me | H | —C(═O)Pr | SEt | C—H | H | CF₃ | H |
| 48 | A | — | Me | H | —C(═O)Pr | SO₂Et | C—H | H | CF₃ | H |
| 49 | A | — | Me | H | —C(═O)—i-Pr | SEt | C—H | H | CF₃ | H |
| 50 | A | — | Me | H | —C(═O)Bu | SEt | C—H | H | CF₃ | H |
| 51 | A | — | Me | H | —C(═O)—i-Pr | SO₂Et | C—H | H | CF₃ | H |
| 52 | A | — | Me | H | —C(═O)Bu | SO₂Et | C—H | H | CF₃ | H |
| 53 | A | — | Me | H | —C(═O)CF₂CF₃ | SO₂Et | C—Cl | H | CF₃ | H |
| 54 | A | — | Me | H | —C(═O)CF₂CF₃ | SO₂Et | C—Br | H | CF₃ | H |
| 55 | A | — | Me | H | —C(═O)CF₂CF₂CF₂CF₃ | SEt | C—H | H | CF₃ | H |
| 56 | A | — | Me | H | —C(═O)CF₂CF₂CF₂CF₃ | SO₂Et | C—H | H | CF₃ | H |
| 57 | A | — | Me | H | —C(═O)CF₂CF₂CF₂CF₃ | SO₂Et | N | H | CF₃ | H |
| 58 | A | — | Me | H | —C(═O)CF₂CF₂H | SEt | C—H | H | CF₃ | H |
| 59 | A | — | Me | H | —C(═O)CF₂CF₂H | SO₂Et | C—H | H | CF₃ | H |
| 60 | A | — | Et | H | H | SEt | C—H | H | CF₃ | H |
| 61 | A | — | Et | H | —C(═O)CF₃ | SEt | C—H | H | CF₃ | H |
| 62 | A | — | Et | H | —C(═O)CF₂CF₃ | SEt | C—H | H | CF₃ | H |
| 63 | A | — | Et | H | —C(═O)CF₂CF₂CF₃ | SEt | C—H | H | CF₃ | H |
| 64 | A | — | Et | H | —C(═O)CF₃ | SO₂Et | C—H | H | CF₃ | H |
| 65 | A | — | Et | H | —C(═O)CF₂CF₃ | SO₂Et | C—H | H | CF₃ | H |
| 66 | A | — | Et | H | —C(═O)CF₂CF₂CF₃ | SO₂Et | C—H | H | CF₃ | H |
| 67 | A | — | Me | H | —C(═O)—c-Pr | SEt | C—H | H | CF₃ | H |
| 68 | A | — | Me | H | —C(═O)—c-Pr | SO₂Et | C—H | H | CF₃ | H |
| 69 | A | — | Me | H | —C(═O)CH₂CF₃ | SEt | C—H | H | CF₃ | H |
| 70 | A | — | Me | H | —C(═O)CH₂CF₃ | SO₂Et | C—H | H | CF₃ | H |
| 71 | A | — | Me | H | —C(═O)—i-Bu | SEt | C—H | H | CF₃ | H |
| 72 | A | — | Me | H | —C(═O)—i-Bu | SO₂Et | C—H | H | CF₃ | H |
| 73 | A | — | Me | H | —C(═O)CH₂Cl | SO₂Et | C—H | H | CF₃ | H |
| 74 | A | — | Me | H | 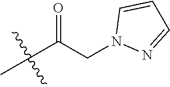 [SMILES: *C(=O)Cn1cccn1] | SO₂Et | C—H | H | CF₃ | H |
| 75 | A | — | Me | H | 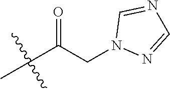 [SMILES: *C(=O)Cn1cncn1] | SO₂Et | C—H | H | CF₃ | H |
| 76 | A | — | Et | H | —C(═O)Me | SEt | C—H | H | CF₃ | H |
| 77 | A | — | Et | H | —C(═O)Me | SO₂Et | C—H | H | CF₃ | H |
| 78 | A | — | Et | H | —C(═O)Et | SEt | C—H | H | CF₃ | H |
| 79 | A | — | Et | H | —C(═O)Pr | SEt | C—H | H | CF₃ | H |
| 80 | A | — | Et | H | —C(═O)Et | SO₂Et | C—H | H | CF₃ | H |
| 81 | A | — | Et | H | —C(═O)Pr | SO₂Et | C—H | H | CF₃ | H |
| 82 | A | — | Et | H | H | SO₂Et | C—H | H | CF₃ | H |
| 83 | A | — | Me | H | 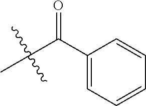 [SMILES: *C(=O)c1ccccc1] | SEt | C—H | H | CF₃ | H |
| 84 | A | — | Me | H | 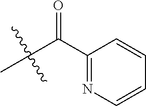 [SMILES: *C(=O)c1ccccn1] | SEt | C—H | H | CF₃ | H |
| 85 | A | — | Me | H | 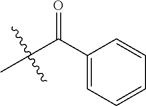 [SMILES: *C(=O)c1ccccc1] | SO₂Et | C—H | H | CF₃ | H |
| 86 | A | — | Me | H | 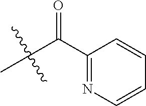 [SMILES: *C(=O)c1ccccn1] | SO₂Et | C—H | H | CF₃ | H |
| 87 | A | — | Me | H | 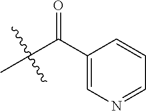 [SMILES: *C(=O)c1cccnc1] | SEt | C—H | H | CF₃ | H |
| 88 | A | — | Et | Me | —C(═O)O—t-Bu | SEt | C—H | H | CF₃ | H |
| 89 | A | — | Et | H | Me | SEt | C—H | H | CF₃ | H |
| 90 | A | — | Et | H | Me | SO₂Et | C—H | H | CF₃ | H |
| 91 | A | — | Me | H | —C(═O)CF₂CF₂CF₂CF₂CF₃ | SEt | C—H | H | CF₃ | H |
| 92 | A | — | Me | H | —C(═O)CF₂CF₂CF₂CF₂CF₃ | SO₂Et | C—H | H | CF₃ | H |
| 93 | A | — | Pr | H | H | SEt | C—H | H | CF₃ | H |
| 94 | A | — | Pr | H | —C(═O)CF₃ | SEt | C—H | H | CF₃ | H |
| 95 | A | — | Pr | H | —C(═O)CF₂CF₃ | SEt | C—H | H | CF₃ | H |
| 96 | A | — | Pr | H | —C(═O)CF₂CF₂CF₃ | SEt | C—H | H | CF₃ | H |
| 97 | A | — | Pr | H | —C(═O)CF₃ | SO₂Et | C—H | H | CF₃ | H |
| 98 | A | — | Pr | H | —C(═O)CF₂CF₃ | SO₂Et | C—H | H | CF₃ | H |
| 99 | A | — | Pr | H | —C(═O)CF₂CF₂CF₃ | SO₂Et | C—H | H | CF₃ | H |
| 100 | A | — | Me | H | H | SO₂Et | C—H | H | CF₃ | H |
| 101 | A | — | Pr | H | —C(═O)Me | SEt | C—H | H | CF₃ | H |
| 102 | A | — | Pr | H | —C(═O)Me | SO₂Et | C—H | H | CF₃ | H |
| 103 | A | — | Pr | H | —C(═O)Et | SEt | C—H | H | CF₃ | H |
| 104 | A | — | Pr | H | —C(═O)Et | SO₂Et | C—H | H | CF₃ | H |
| 105 | A | — | Pr | H | —C(═O)Pr | SEt | C—H | H | CF₃ | H |
| 106 | A | — | Pr | H | —C(═O)Pr | SO₂Et | C—H | H | CF₃ | H |
| 107 | A | — | Me | H | —C(═O)CF₂Me | SEt | C—H | H | CF₃ | H |
| 108 | A | — | Me | —C(═O)Me | —C(═O)Me | SEt | C—H | H | CF₃ | H |
| 109 | A | — | Me | H | —C(═O)CF₂Me | SO₂Et | C—H | H | CF₃ | H |
| 110 | A | — | Me | —C(═O)Me | —C(═O)Me | SO₂Et | C—H | H | CF₃ | H |
| 111 | A | — | Et | H | —C(═O)CF₂H | SEt | C—H | H | CF₃ | H |
| 112 | A | — | Et | H | —C(═O)CF₂H | SO₂Et | C—H | H | CF₃ | H |
| 113 | A | — | Et | H | —C(═O)CF₂CF₂H | SEt | C—H | H | CF₃ | H |
| 114 | A | — | Pr | H | —C(═O)CF₂H | SEt | C—H | H | CF₃ | H |
| 115 | A | — | Et | H | —C(═O)CF₂CF₂H | SO₂Et | C—H | H | CF₃ | H |
| 116 | A | — | Pr | H | —C(═O)CF₂H | SO₂Et | C—H | H | CF₃ | H |
| 117 | A | — | Me | H | —C(═O)CClF₂ | SEt | C—H | H | CF₃ | H |
| 118 | A | — | Me | H | —C(═O)CHCl₂ | SEt | C—H | H | CF₃ | H |
| 119 | A | — | Me | H | —C(═O)CClF₂ | SO₂Et | C—H | H | CF₃ | H |
| 120 | A | — | Me | H | —C(═O)CHCl₂ | SO₂Et | C—H | H | CF₃ | H |
| 121 | A | — | Pr | H | —C(═O)CF₂CF₂H | SEt | C—H | H | CF₃ | H |
| 122 | A | — | Pr | H | —C(═O)CF₂CF₂H | SO₂Et | C—H | H | CF₃ | H |
| 123 | A | — | Me | H | —C(═O)CF₃ | SO₂Et | C—Br | H | CF₃ | H |
| 124 | A | — | Me | H | —C(═O)CF₃ | SO₂Et | C—Cl | H | CF₃ | H |
| 125 | A | — | Me | H | —C(═O)CF₂CClF₂ | SEt | C—H | H | CF₃ | H |
| 126 | A | — | Me | H | —C(═O)CF₂CClF₂ | SO₂Et | C—H | H | CF₃ | H |
| 127 | A | — | Et | Me | —C(═O)CF₃ | SO₂Et | C—H | H | CF₃ | H |
| 128 | A | — | 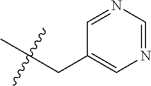 [SMILES: *Cc1cncnc1] | H | H | SEt | C—H | H | CF₃ | H |
| 129 | A | — | 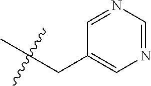 [SMILES: *Cc1cncnc1] | H | —C(═O)CF₃ | SEt | C—H | H | CF₃ | H |
| 130 | A | — | 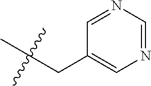 [SMILES: *Cc1cncnc1] | H | —C(═O)CF₃ | SO₂Et | C—H | H | CF₃ | H |
| 131 | A | — | Me | H | —C(═O)CCl₃ | SEt | C—H | H | CF₃ | H |
| 132 | A | — | Me | H | —C(═O)CCl₃ | SO₂Et | C—H | H | CF₃ | H |
| 133 | A | — | Me | H | —C(═O)NMez | SEt | C—H | H | CF₃ | H |
| 134 | A | — | Me | H | —C(═O)OCH₂CF₃ | SEt | C—H | H | CF₃ | H |
| 135 | A | — | Me | H | —C(═O)CH₂CF₂H | SEt | C—H | H | CF₃ | H |
| 136 | A | — | Me | H | —C(═O)CH₂CF₂H | SO₂Et | C—H | H | CF₃ | H |
| 137 | B | CH₂CF₃ | — | H | —C(═O)CF₃ | SEt | C—H | H | CF₃ | H |
| 138 | B | CH₂CF₃ | — | H | —C(═O)CF₃ | SO₂Et | C—H | H | CF₃ | H |
| 139 | A | — | Me | H | 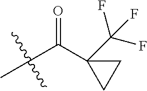 [SMILES: *C(=O)C1(C(F)(F)F)CC1] | SEt | C—H | H | CF₃ | H |
| 140 | B | 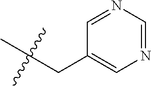 [SMILES: *Cc1cncnc1] | H | H | —C(═O)CF₃ | SEt | C—H | H | CF₃ | H |
| 141 | B | 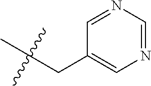 [SMILES: *Cc1cncnc1] | H | H | —C(═O)CF₃ | SO₂Et | C—H | H | CF₃ | H |
| 142 | B | 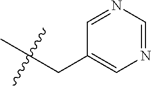 [SMILES: *Cc1cncnc1] | H | H | —C(═O)CF₂CF₂CF₃ | SEt | C—H | H | CF₃ | H |
| 143 | B | 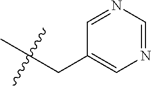 [SMILES: *Cc1cncnc1] | H | H | —C(═O)CF₂CF₂CF₃ | SO₂Et | C—H | H | CF₃ | H |
| 144 | A | — | Me | H | —C(═O)OCH₂CF₃ | SO₂Et | C—H | H | CF₃ | H |
| 145 | A | — | Me | H | 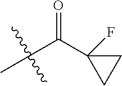 [SMILES: *C(=O)C1(F)CC1] | SEt | C—H | H | CF₃ | H |
| 146 | A | — | Me | H | 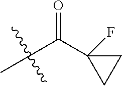 [SMILES: *C(=O)C1(F)CC1] | SO₂Et | C—H | H | CF₃ | H |
| 147 | A | — | Me | H | —C(═O)CCl₂F | SEt | C—H | H | CF₃ | H |
| 148 | A | — | Me | H | —SO₂Me | SEt | C—H | H | CF₃ | H |
| 149 | A | — | CH₂CF₃ | H | H | SEt | C—H | H | CF₃ | H |
| 150 | A | — | CH₂CF₃ | H | —C(═O)CF₃ | SEt | C—H | H | CF₃ | H |
| 151 | A | — | CH₂CF₃ | H | —C(═O)CF₃ | SO₂Et | C—H | H | CF₃ | H |
| 152 | A | — | Me | H | —C(═O)NMe₂ | SEt | C—H | H | CF₃ | H |
| 153 | A | — | Me | H | —C(═O)CCl₂F | SO₂Et | C—H | H | CF₃ | H |
| 154 | A | — | Me | H | —SO₂Me | SO₂Et | C—H | H | CF₃ | H |
| 155 | A | — | CH₂CF₃ | H | —C(═O)CF₂CF₃ | SEt | C—H | H | CF₃ | H |
| 156 | A | — | CH₂CF₃ | H | —C(═O)CF₂CF₃ | SO₂Et | C—H | H | CF₃ | H |
| 157 | A | — | CH₂CF₃ | H | —C(═O)CF₂CF₂CF₃ | SEt | C—H | H | CF₃ | H |
| 158 | A | — | CH₂CF₃ | H | —C(═O)CF₂CF₂CF₃ | SO₂Et | C—H | H | CF₃ | H |
| 159 | A | — | CH₂CF₃ | H | —C(═O)Me | SEt | C—H | H | CF₃ | H |
| 160 | A | — | CH₂CF₃ | H | —C(═O)Me | SO₂Et | C—H | H | CF₃ | H |
| 161 | A | — | CH₂CF₃ | H | —C(═O)Et | SEt | C—H | H | CF₃ | H |
| 162 | A | — | CH₂CF₃ | H | —C(═O)Et | SO₂Et | C—H | H | CF₃ | H |
| 163 | A | — | CH₂CF₃ | H | —C(═O)Pr | SEt | C—H | H | CF₃ | H |
| 164 | A | — | CH₂CF₃ | H | —C(═O)Pr | SO₂Et | C—H | H | CF₃ | H |
| 165 | A | — | Me | H | —C(═O)CH═CH₂ | SEt | C—H | H | CF₃ | H |
| 166 | A | — | Me | H | —C(═O)CH═CH₂ | SO₂Et | C—H | H | CF₃ | H |
| 167 | A | — | Me | H | —C(═O)NMe₂ | SO₂Et | C—H | H | CF₃ | H |
| 168 | A | — | Et | H | —C(═O)O-t-Bu | SEt | N | H | CF₃ | H |
| 169 | A | — | Me | H | —C(═O)CF₃ | SO₂Et | C—Me | H | CF₃ | H |
| 170 | A | — | Me | H | —C(═O)CF═CH₂ | SEt | C—H | H | CF₃ | H |
| 171 | A | — | Et | H | —C(═O)CF₃ | SEt | N | H | CF₃ | H |
| 172 | A | — | Et | H | —C(═O)CF₃ | SO₂Et | N | H | CF₃ | H |
| 173 | A | — | Me | H | —C(═O)CF═CH₂ | SO₂Et | C—H | H | CF₃ | H |
| 174 | A | — | Me | H | —C(═O)NHMe | SEt | C—H | H | CF₃ | H |
| 175 | A | — | Et | H | —C(═O)CF₂CF₃ | SEt | N | H | CF₃ | H |
| 176 | A | — | Et | H | —C(═O)CF₂CF₃ | SO₂Et | N | H | CF₃ | H |
| 177 | A | — | Et | H | —C(═O)CF₂CF₂CF₃ | SEt | N | H | CF₃ | H |
| 178 | A | — | Et | H | —C(═O)CF₂CF₂CF₃ | SO₂Et | N | H | CF₃ | H |
| 179 | A | — | Me | H | —C(═O)CCl₃ | SEt | N | H | CF₃ | H |
| 180 | A | — | Me | H | —C(═O)CF₂CF₂H | SEt | N | H | CF₃ | H |
| 181 | A | — | Me | H | H | SO₂Et | N | H | CF₃ | H |
| 182 | A | — | Me | H | —C(═O)CCl₃ | SO₂Et | N | H | CF₃ | H |
| 183 | A | — | Me | H | —C(═O)CF₂CF₂H | SO₂Et | N | H | CF₃ | H |
| 184 | A | — | Et | H | —C(═O)CF₂CF₂H | SEt | N | H | CF₃ | H |
| 185 | A | — | Et | H | —C(═O)CF₂CF₂H | SO₂Et | N | H | CF₃ | H |
| 186 | A | — | Et | H | —C(═O)CCl₃ | SEt | N | H | CF₃ | H |
| 187 | A | — | Et | H | —C(═O)CCl₃ | SO₂Et | N | H | CF₃ | H |
| 188 | A | — | Et | H | —C(═O)Me | SEt | N | H | CF₃ | H |
| 189 | A | — | Et | H | —C(═O)Me | SO₂Et | N | H | CF₃ | H |
| 190 | A | — | Me | H | 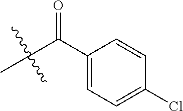 [SMILES: *C(=O)c1ccc(Cl)cc1] | SEt | C—H | H | CF₃ | H |
| 191 | A | — | Me | H | 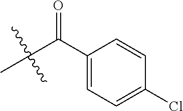 [SMILES: *C(=O)c1ccc(Cl)cc1] | SO₂Et | C—H | H | CF₃ | H |
| 192 | A | — | Me | H | 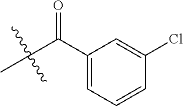 [SMILES: *C(=O)c1cccc(Cl)c1] | SEt | C—H | H | CF₃ | H |
| 193 | A | — | Me | H | H | SEt | C—H | Me | CF₃ | H |
| 194 | A | — | Me | H | —C(═O)CF₂CF₂CF₃ | SEt | C—H | Me | CF₃ | H |
| 195 | A | — | Me | H | 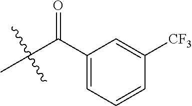 [SMILES: *C(=O)c1cccc(C(F)(F)F)c1] | SEt | C—H | H | CF₃ | H |
| 196 | A | — | Me | H | 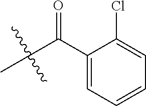 [SMILES: *C(=O)c1ccccc1Cl] | SEt | C—H | H | CF₃ | H |
| 197 | A | — | Me | H | 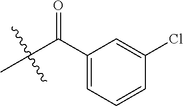 [SMILES: *C(=O)c1cccc(Cl)c1] | SO₂Et | C—H | H | CF₃ | H |
| 198 | A | — | Me | H | 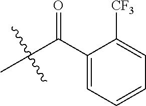 [SMILES: *C(=O)c1ccccc1C(F)(F)F] | SEt | C—H | H | CF₃ | H |
| 199 | A | — | Me | H | 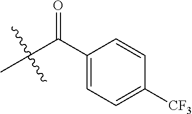 [SMILES: *C(=O)c1ccc(C(F)(F)F)cc1] | SEt | C—H | H | CF₃ | H |
| 200 | A | — | Me | H | 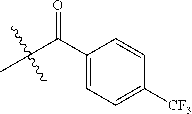 [SMILES: *C(=O)c1ccc(C(F)(F)F)cc1] | SO₂Et | C—H | H | CF₃ | H |
| 201 | A | — | Me | H | 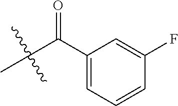 [SMILES: *C(=O)c1cccc(F)c1] | SEt | C—H | H | CF₃ | H |
| 202 | A | — | Me | H | 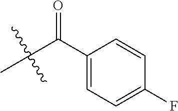 [SMILES: *C(=O)c1ccc(F)cc1] | SEt | C—H | H | CF₃ | H |
| 203 | A | — | Me | H | 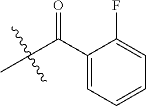 [SMILES: *C(=O)c1ccccc1F] | SEt | C—H | H | CF₃ | H |
| 204 | A | — | Me | H | 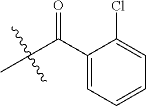 [SMILES: *C(=O)c1ccccc1Cl] | SO₂Et | C—H | H | CF₃ | H |
| 205 | A | — | Me | H | —C(═O)CF₂CF₂CF₃ | SO₂Et | C—H | H | CF₃ | H |
| 206 | A | — | Me | H | 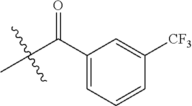 [SMILES: *C(=O)c1cccc(C(F)(F)F)c1] | SO₂Et | C—H | H | CF₃ | H |
| 207 | A | — | Me | H | 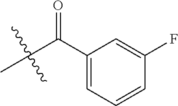 [SMILES: *C(=O)c1cccc(F)c1] | SO₂Et | C—H | H | CF₃ | H |
| 208 | A | — | Me | H | 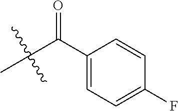 [SMILES: *C(=O)c1ccc(F)cc1] | SO₂Et | C—H | H | CF₃ | H |
| 209 | A | — | Me | H | 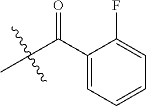 [SMILES: *C(=O)c1ccccc1F] | SO₂Et | C—H | H | CF₃ | H |
| 210 | A | — | Me | H | 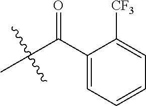 [SMILES: *C(=O)c1ccccc1C(F)(F)F] | SO₂Et | C—H | H | CF₃ | H |
| 211 | A | — | Me | H | 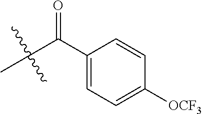 [SMILES: *C(=O)c1ccc(OC(F)(F)F)cc1] | SEt | C—H | H | CF₃ | H |
| 212 | A | — | Me | H | 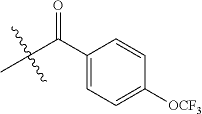 [SMILES: *C(=O)c1ccc(OC(F)(F)F)cc1] | SO₂Et | C—H | H | CF₃ | H |
| 213 | A | — | Me | —C(═O)—(4-OCF₃—Ph) | 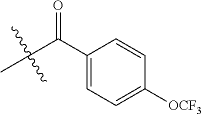 [SMILES: *C(=O)c1ccc(OC(F)(F)F)cc1] | SEt | C—H | H | CF₃ | H |
| 214 | A | — | Me | —C(═O)—c-Hex | —C(═O)—c-Hex | SEt | C—H | H | CF₃ | H |
| 215 | A | — | Me | H | —C(═O)—c-Bu | SEt | C—H | H | CF₃ | H |
| 216 | A | — | Me | H | —C(═O)—c-Hex | SO₂Et | C—H | H | CF₃ | H |
| 217 | A | — | Me | H | —C(═O)—c-Pent | SO₂Et | C—H | H | CF₃ | H |
| 218 | A | — | Me | H | —C(═O)—c-Bu | SO₂Et | C—H | H | CF₃ | H |
| 219 | A | — | Me | H | —C(═O)CF₂CF₃ | SEt | C—H | Me | CF₃ | H |
| 220 | A | — | Me | H | —C(═O)CF₃ | SEt | C—H | Me | CF₃ | H |
| 221 | A | — | Me | H | —C(═O)Me | SEt | C—H | Me | CF₃ | H |
| 222 | A | — | Me | H | —C(═O)CF₂CF₃ | SO₂Et | C—H | Me | CF₃ | H |
| 223 | A | — | Me | H | —C(═O)Me | SO₂Et | C—H | Me | CF₃ | H |
| 224 | A | — | Me | H | —C(═O)CF₃ | SEt | C—H | SMe | CF₃ | H |
| 225 | A | — | Me | H | —C(═O)CCl₃ | SEt | C—H | Me | CF₃ | H |
| 226 | A | — | Me | H | —C(═O)CF₂CF₂H | SEt | C—H | Me | CF₃ | H |
| 227 | A | — | Me | H | —C(═O)CF₃ | SO₂Et | C—H | Me | CF₃ | H |
| 228 | A | — | Me | H | —C(═O)CF₂CF₂H | SO₂Et | C—H | Me | CF₃ | H |
| 229 | A | — | Me | H | —C(═O)CF₃ | SEt | C—H | H | CF₂CF₃ | H |
| 230 | A | — | Me | H | —C(═O)CCl₃ | SO₂Et | C—H | Me | CF₃ | H |
| 231 | A | — | Me | H | —C(═O)CF₃ | SEt | C—H | H | CHF₂ | H |
| 232 | A | — | Me | H | —C(═O)CF₃ | SO₂Et | C—H | H | CF₂CF₃ | H |
| 233 | A | — | Me | H | —C(═O)CF₃ | SO₂Et | C—H | SO₂Me | CF₃ | H |
| 234 | A | — | Me | H | —C(═O)CF₃ | SOEt | C—H | SO₂Me | CF₃ | H |
| 235 | A | — | Me | H | —C(═O)CF₂CF₂CF₃ | SEt | C—H | H | CHF₂ | H |
| 236 | A | — | Me | H | —C(═O)CF₂CF₂H | SEt | C—H | H | CF₂CF₃ | H |
| 237 | A | — | Me | H | —C(═O)CF₂CF₂H | SO₂Et | C—H | H | CF₂CF₃ | H |
| 238 | A | — | Me | H | —C(═O)CF₃ | SO₂Et | C—H | H | CHF₂ | H |
| 239 | A | — | Me | H | —C(═O)CF₂CF₂H | SEt | C—H | H | CHF₂ | H |
| 240 | A | — | Me | H | —C(═O)CF₂CF₂H | SO₂Et | C—H | H | CHF₂ | H |
| 241 | A | — | Me | H | —C(═O)CF₂CF₂CF₃ | SO₂Et | C—H | H | CHF₂ | H |
| 242 | A | — | Me | H | —C(═O)CF₂CF₃ | SEt | C—H | H | CHF₂ | H |
| 243 | A | — | Me | H | —C(═O)CF₂CF₃ | SO₂Et | C—H | H | CHF₂ | H |
| 244 | A | — | Me | H | —C(═O)CF₃ | SEt | C—H | Cl | CF₃ | H |
| 245 | A | — | Me | H | —C(═O)CF₃ | SEt | C—H | OMe | CF₃ | H |
| 246 | A | — | Me | H | —C(═O)CF₃ | SO₂Et | C—H | OMe | CF₃ | H |
| 247 | A | — | Et | H | —C(═O)CCl₂F | SEt | C—H | H | CF₃ | H |
| 248 | A | — | Et | H | —C(═O)CCl₂F | SO₂Et | C—H | H | CF₃ | H |
| 249 | A | — | Me | H | —C(═O)CF₃ | SO₂Et | C—H | Cl | CF₃ | H |
| 250 | A | — | Me | H | —C(═O)CCl₃ | SEt | C—H | H | CHF₂ | H |
| 251 | A | — | Me | H | —C(═O)CF₂CF₃ | SEt | C—H | H | CF₂CF₃ | H |
| 252 | A | — | Et | H | —C(═O)CCl₃ | SEt | C—H | H | CF₃ | H |
| 253 | A | — | Et | H | —C(═O)CCl₃ | SO₂Et | C—H | H | CF₃ | H |
| 254 | A | — | Me | H | —C(═O)CCl₃ | SO₂Et | C—H | H | CHF₂ | H |
| 255 | A | — | Me | H | —C(═O)CF₂CF₃ | SO₂Et | C—H | H | CF₂CF₃ | H |
| 256 | A | — | Me | H | —C(═O)CF₂CF₂CF₃ | SEt | C—H | H | CF₂CF₃ | H |
| 257 | A | — | Me | H | —C(═O)CF₂CF₂CF₃ | SO₂Et | C—H | H | CF₂CF₃ | H |
| 258 | A | — | Me | H | —C(═O)O—t-Bu | SEt | C—H | c-Pr | CF₃ | H |
| 259 | A | — | Me | H | H | SEt | C—H | c-Pr | CF₃ | H |
| 260 | A | — | Me | H | —C(═O)CF₃ | SEt | C—H | c-Pr | CF₃ | H |
| 261 | A | — | Me | H | —C(═O)CF₂CF₂H | SEt | C—H | c-Pr | CF₃ | H |
| 262 | A | — | Me | H | —C(═O)CF₂CF₃ | SEt | C—H | c-Pr | CF₃ | H |
| 263 | A | — | Me | H | H | SO₂Et | C—H | c-Pr | CF₃ | H |
| 264 | A | — | Me | H | —C(═O)CF₃ | SO₂Et | C—H | c-Pr | CF₃ | H |
| 265 | A | — | Me | H | —C(═O)CF₂CF₂H | SO₂Et | C—H | c-Pr | CF₃ | H |
| 266 | A | — | Me | H | —C(═O)CF₂CF₃ | SO₂Et | C—H | c-Pr | CF₃ | H |
| 267 | A | — | Me | H | —C(═O)CCl₃ | SEt | C—H | H | CF₂CF₃ | H |
| 268 | A | — | Me | H | —C(═O)CF₃ | SEt | C—H | OEt | CF₃ | H |
| 269 | A | — | Me | H | —C(═O)CF₃ | SO₂Et | C—H | OEt | CF₃ | H |
| 270 | A | — | Me | H | —C(═O)CF₂CF₂CF₃ | SEt | C—H | c-Pr | CF₃ | H |
| 271 | A | — | Me | H | —C(═O)CF₂CF₂CF₃ | SO₂Et | C—H | c-Pr | CF₃ | H |
| 272 | A | — | Me | H | —C(═O)CF₃ | SO₂Et | C—H | OPr | CF₃ | H |
| 273 | A | — | Me | H | —C(═O)CCl₃ | SO₂Et | C—H | H | CF₂CF₃ | H |
| 274 | A | — | Et | H | —C(═O)CF₃ | SEt | C—H | H | CF₂CF₃ | H |
| 275 | A | — | Et | H | —C(═O)CF₃ | SO₂Et | C—H | H | CF₂CF₃ | H |
| 276 | A | — | Me | H | —C(═O)CF₃ | SEt | C—H | OCH₂CF₃ | CF₃ | H |
| 277 | A | — | Me | CH₂CF₃ | —C(═O)CF₃ | SEt | C—H | OH | CF₃ | H |
| 278 | A | — | Me | H | —C(═O)CF₃ | SO₂Et | C—H | OCH₂CF₃ | CF₃ | H |
| 279 | A | — | Me | H | —C(═O)O—t-Bu | SEt | C—H | H | CF₂CF₃ | H |
| 280 | A | — | Me | H | 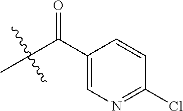 [SMILES: *C(=O)c1ccc(Cl)nc1] | SEt | C—H | H | CF₃ | H |
| 281 | A | — | Me | H | H | SEt | C—H | H | CF₂CF₃ | H |
| 282 | A | — | Me | H | 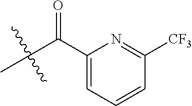 [SMILES: *C(=O)c1cccc(C(F)(F)F)n1] | SEt | C—H | H | CF₃ | H |
| 283 | A | — | Me | H | 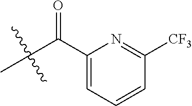 [SMILES: *C(=O)c1cccc(C(F)(F)F)n1] | SO₂Et | C—H | H | CF₃ | H |
| 284 | A | — | Me | H | 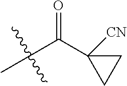 [SMILES: *C(=O)C1(C#N)CC1] | SEt | C—H | H | CF₃ | H |
| 285 | A | — | Me | H | 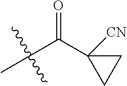 [SMILES: *C(=O)C1(C#N)CC1] | SO₂Et | C—H | H | CF₃ | H |
| 286 | A | — | Et | H | H | SEt | C—H | H | CF₂CF₃ | H |
| 287 | A | — | Et | H | —C(═O)O—t-Bu | SEt | C—H | H | CF₂CF₃ | H |
| 288 | A | — | Et | H | 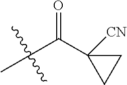 [SMILES: *C(=O)C1(C#N)CC1] | SEt | C—H | H | CF₃ | H |
| 289 | A | — | Et | H | 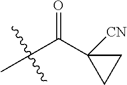 [SMILES: *C(=O)C1(C#N)CC1] | SO₂Et | C—H | H | CF3 | H |
| 290 | A | — | Et | H | —C(═O)CF₂CF₃ | SEt | C—H | H | CF₂CF₃ | H |
| 291 | A | — | Et | H | —C(═O)CF₂CF₂CF₃ | SEt | C—H | H | CF₂CF₃ | H |
| 292 | A | — | Et | H | —C(═O)CF₂CF₃ | SO₂Et | C—H | H | CF₂CF₃ | H |
| 293 | A | — | Et | H | H | SO₂Et | C—H | H | CF₂CF₃ | H |
| 294 | A | — | Et | H | —C(═O)CF₂CF₂CF₃ | SO₂Et | C—H | H | CF₂CF₃ | H |
| 295 | A | — | Me | H | 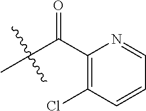 [SMILES: *C(=O)c1ncccc1Cl] | SEt | C—H | H | CF₃ | H |
| 296 | A | — | Me | H | 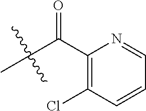 [SMILES: *C(=O)c1ncccc1Cl] | SO₂Et | C—H | H | CF₃ | H |
| 297 | A | — | Me | H | 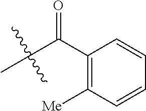 [SMILES: *C(=O)c1ccccc1C] | SEt | C—H | H | CF₃ | H |
| 298 | A | — | Me | H | 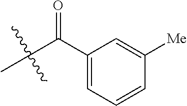 [SMILES: *C(=O)c1cccc(C)c1] | SEt | C—H | H | CF₃ | H |
| 299 | A | — | Me | H | 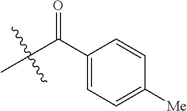 [SMILES: *C(=O)c1ccc(C)cc1] | SEt | C—H | H | CF₃ | H |
| 300 | A | — | Me | H | 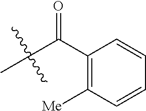 [SMILES: *C(=O)c1ccccc1C] | SO₂Et | C—H | H | CF₃ | H |
| 301 | A | — | Me | H | 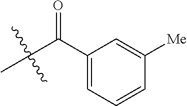 [SMILES: *C(=O)c1cccc(C)c1] | SO₂Et | C—H | H | CF₃ | H |
| 302 | A | — | Me | H | 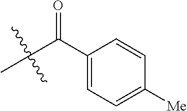 [SMILES: *C(=O)c1ccc(C)cc1] | SO₂Et | C—H | H | CF₃ | H |
| 303 | A | — | Me | H | —C(═O)CF₃ | SEt | C—H | OH | CF₃ | H |
| 304 | A | — | Me | H | —C(═O)CF₃ | SO₂Et | C—H | OH | CF₃ | H |
| 305 | A | — | Me | CH2CF3 | —C(═O)CF₃ | SO₂Et | C—H | OH | CF₃ | H |
| 306 | A | — | Et | H | 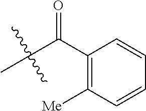 [SMILES: *C(=O)c1ccccc1C] | SEt | C—H | H | CF₃ | H |
| 307 | A | — | Et | H | 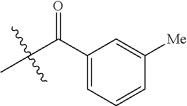 [SMILES: *C(=O)c1cccc(C)c1] | SEt | C—H | H | CF₃ | H |
| 308 | A | — | Et | H | 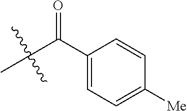 [SMILES: *C(=O)c1ccc(C)cc1] | SEt | C—H | H | CF₃ | H |
| 309 | A | — | Et | H | 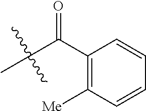 [SMILES: *C(=O)c1ccccc1C] | SO₂Et | C—H | H | CF₃ | H |
| 310 | A | — | Et | H | 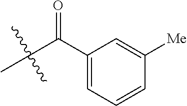 [SMILES: *C(=O)c1cccc(C)c1] | SO₂Et | C—H | H | CF₃ | H |
| 311 | A | — | Et | H | 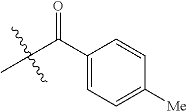 [SMILES: *C(=O)c1ccc(C)cc1] | SO₂Et | C—H | H | CF₃ | H |
| 312 | A | — | Me | H | 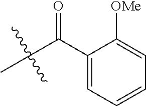 [SMILES: *C(=O)c1ccccc1OC] | SEt | C—H | H | CF₃ | H |
| 313 | A | — | Me | H | 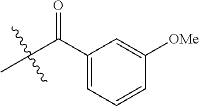 [SMILES: *C(=O)c1cccc(OC)c1] | SEt | C—H | H | CF₃ | H |
| 314 | A | — | Me | H | 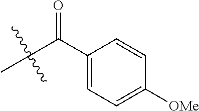 [SMILES: *C(=O)c1ccc(OC)cc1] | SEt | C—H | H | CF₃ | H |
| 315 | A | — | Me | H | 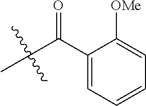 [SMILES: *C(=O)c1ccccc1OC] | SO₂Et | C—H | H | CF₃ | H |
| 316 | A | — | Me | H | 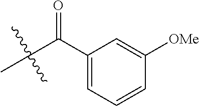 [SMILES: *C(=O)c1cccc(OC)c1] | SO₂Et | C—H | H | CF₃ | H |
| 317 | A | — | Me | H | 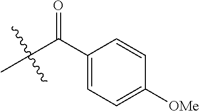 [SMILES: *C(=O)c1ccc(OC)cc1] | SO₂Et | C—H | H | CF₃ | H |
| 318 | A | — | Et | H | 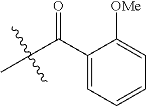 [SMILES: *C(=O)c1ccccc1OC] | SEt | C—H | H | CF₃ | H |
| 319 | A | — | Et | H | 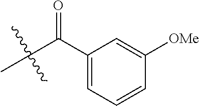 [SMILES: *C(=O)c1cccc(OC)c1] | SEt | C—H | H | CF₃ | H |
| 320 | A | — | Et | H | 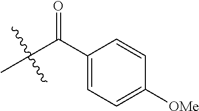 [SMILES: *C(=O)c1ccc(OC)cc1] | SEt | C—H | H | CF₃ | H |
| 321 | A | — | Et | H | 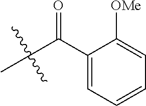 [SMILES: *C(=O)c1ccccc1OC] | SO₂Et | C—H | H | CF₃ | H |
| 322 | A | — | Et | H | 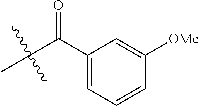 [SMILES: *C(=O)c1cccc(OC)c1] | SO₂Et | C—H | H | CF₃ | H |
| 323 | A | — | Et | H | 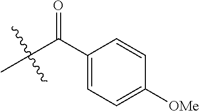 [SMILES: *C(=O)c1ccc(OC)cc1] | SO₂Et | C—H | H | CF₃ | H |
| 324 | A | — | Et | H | —C(═O)CF₂CF₂H | SEt | C—H | H | CF₂CF₃ | H |
| 325 | A | — | Et | H | —C(═O)CCl₃ | SEt | C—H | H | CF₂CF₃ | H |
| 326 | A | — | Me | H | —C(═O)CF₃ | SEt | C—H | H | CF₂CF₂CF₃ | H |
| 327 | A | — | Me | H | —C(═O)CF₂CF₂H | SEt | C—H | H | CF₂CF₂CF₃ | H |
| 328 | A | — | Et | H | —C(═O)CF₂CF₂H | SO₂Et | C—H | H | CF₂CF₃ | H |
| 329 | A | — | Et | H | —C(═O)CCl₃ | SO₂Et | C—H | H | CF₂CF₃ | H |
| 330 | A | — | c-Pr | H | H | SEt | C—H | H | CF₃ | H |
| 331 | A | — | Me | H | 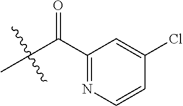 [SMILES: *C(=O)c1cc(Cl)ccn1] | SEt | C—H | H | CF₃ | H |
| 332 | A | — | Me | H | 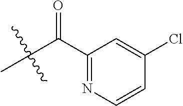 [SMILES: *C(=O)c1cc(Cl)ccn1] | SO₂Et | C—H | H | CF₃ | H |
| 333 | A | — | Me | H | 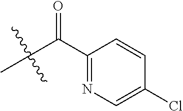 [SMILES: *C(=O)c1ccc(Cl)cn1] | SEt | C—H | H | CF₃ | H |
| 334 | A | — | Me | H | 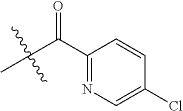 [SMILES: *C(=O)c1ccc(Cl)cn1] | SO₂Et | C—H | H | CF₃ | H |
| 335 | A | — | Me | H | 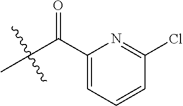 [SMILES: *C(=O)c1cccc(Cl)n1] | SEt | C—H | H | CF₃ | H |
| 336 | A | — | Me | H | 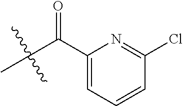 [SMILES: *C(=O)c1cccc(Cl)n1] | SO₂Et | C—H | H | CF₃ | H |
| 337 | A | — | Me | H | 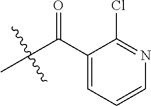 [SMILES: *C(=O)c1cccnc1Cl] | SEt | C—H | H | CF₃ | H |
| 338 | A | — | Me | H | 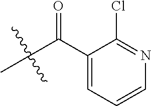 [SMILES: *C(=O)c1cccnc1Cl] | SO₂Et | C—H | H | CF₃ | H |
| 339 | A | — | Me | H | 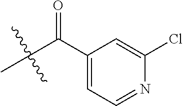 [SMILES: *C(=O)c1ccnc(Cl)c1] | SEt | C—H | H | CF₃ | H |
| 340 | A | — | Me | H | 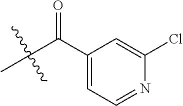 [SMILES: *C(=O)c1ccnc(Cl)c1] | SO₂Et | C—H | H | CF₃ | H |
| 341 | A | — | c-Pr | H | —C(═O)CF₃ | SEt | C—H | H | CF₃ | H |
| 342 | A | — | Me | H | —C(═O)CF₃ | SO₂Et | C—H | H | CF₂CF₂CF₃ | H |
| 343 | A | — | Me | H | —C(═O)CF₂CF₂H | SO₂Et | C—H | H | CF₂CF₂CF₃ | H |
| 344 | A | — | c-Pr | H | —C(═O)CF₃ | SO₂Et | C—H | H | CF₃ | H |
| 345 | A | — | Me | H | 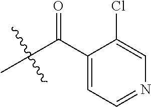 [SMILES: *C(=O)c1ccncc1Cl] | SEt | C—H | H | CF₃ | H |
| 346 | A | — | Me | H | 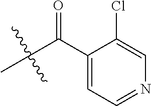 [SMILES: *C(=O)c1ccncc1Cl] | SO₂Et | C—H | H | CF₃ | H |
| 347 | A | — | Me | H | 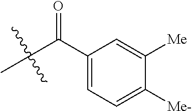 | SEt | C—H | H | CF₃ | H |
| 348 | A | — | Me | H | 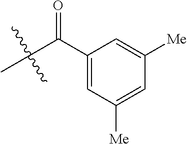 [SMILES: *C(=O)c1cc(C)cc(C)c1] | SEt | C—H | H | CF₃ | H |
| 349 | A | — | Me | H | 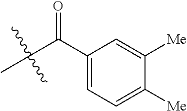 [SMILES: *C(=O)c1ccc(C)c(C)c1] | SO₂Et | C—H | H | CF₃ | H |
| 350 | A | — | Me | H | 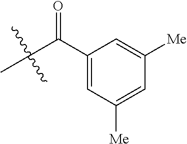 [SMILES: *C(=O)c1cc(C)cc(C)c1] | SO₂Et | C—H | H | CF₃ | H |
| 351 | A | — | c-Pr | H | H | SO₂Et | C—H | H | CF₃ | H |
| 352 | A | — | Me | H | 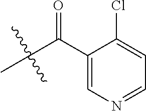 [SMILES: *C(=O)c1cnccc1Cl] | SEt | C—H | H | CF₃ | H |
| 353 | A | — | Me | H | 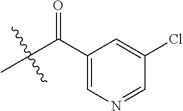 [SMILES: *C(=O)c1cncc(Cl)c1] | SEt | C—H | H | CF₃ | H |
| 354 | A | — | Me | H | 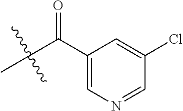 [SMILES: *C(=O)c1cncc(Cl)c1] | SO₂Et | C—H | H | CF₃ | H |
| 355 | A | — | Me | H | 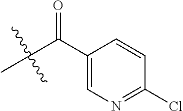 [SMILES: *C(=O)c1ccc(Cl)nc1] | SO₂Et | C—H | H | CF₃ | H |
| 356 | A | — | Me | H | 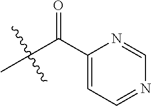 [SMILES: *C(=O)c1ccncn1] | SEt | C—H | H | CF₃ | H |
| 357 | A | — | Me | H | 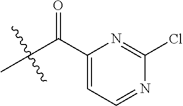 [SMILES: *C(=O)c1ccnc(Cl)n1] | SEt | C—H | H | CF₃ | H |
| 358 | A | — | Me | H | 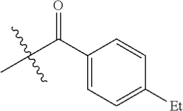 [SMILES: *C(=O)c1ccc(CC)cc1] | SEt | C—H | H | CF₃ | H |
| 359 | A | — | Me | H | 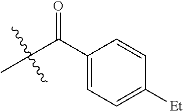 [SMILES: *C(=O)c1ccc(CC)cc1] | SO₂Et | C—H | H | CF₃ | H |
| 360 | A | — | Me | H | 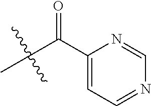 [SMILES: *C(=O)c1ccncn1] | SO₂Et | C—H | H | CF₃ | H |
| 361 | A | — | Me | H | 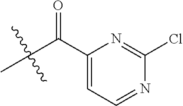 [SMILES: *C(=O)c1ccnc(Cl)n1] | SO₂Et | C—H | H | CF₃ | H |
| 362 | A | — | Me | H | 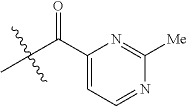 [SMILES: *C(=O)c1ccnc(C)n1] | SEt | C—H | H | CF₃ | H |
| 363 | A | — | Me | H | 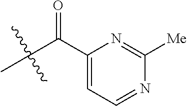 [SMILES: *C(=O)c1ccnc(C)n1] | SO₂Et | C—H | H | CF₃ | H |
| 364 | A | — | Me | H | 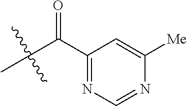 [SMILES: *C(=O)c1cc(C)ncn1] | SEt | C—H | H | CF₃ | H |
| 365 | A | — | Me | H | 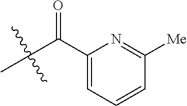 [SMILES: *C(=O)c1cccc(C)n1] | SEt | C—H | H | CF₃ | H |
| 366 | A | — | Me | H | 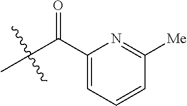 [SMILES: *C(=O)c1cccc(C)n1] | SO₂Et | C—H | H | CF₃ | H |
| 367 | A | — | Me | H | —C(═O)Me | SEt | C—H | H | CF₃ | H |
| 368 | A | — | c-Pr | H | —C(═O)CF₂CF₃ | SEt | C—H | H | CF₃ | H |
| 369 | A | — | c-Pr | H | —C(═O)CF₂CF₂CF₃ | SEt | C—H | H | CF₃ | H |
| 370 | A | — | c-Pr | H | —C(═O)CF₂CF₃ | SO₂Et | C—H | H | CF₃ | H |
| 371 | A | — | c-Pr | H | —C(═O)CF₂CF₂CF₃ | SO₂Et | C—H | H | CF₃ | H |
| 372 | A | — | Me | H | 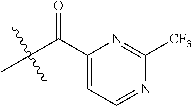 [SMILES: *C(=O)c1ccnc(C(F)(F)F)n1] | SEt | C—H | H | CF₃ | H |
| 373 | A | — | Me | H | 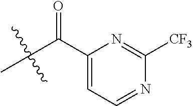 [SMILES: *C(=O)c1ccnc(C(F)(F)F)n1] | SO₂Et | C—H | H | CF₃ | H |
| 374 | A | — | Me | H | 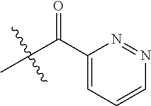 [SMILES: *C(=O)c1cccnn1] | SEt | C—H | H | CF₃ | H |
| 375 | A | — | Me | H | 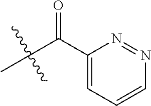 [SMILES: *C(=O)c1cccnn1] | SO₂Et | C—H | H | CF₃ | H |
| 376 | A | — | Me | H | —C(═O)CF₂CF₃ | SEt | C—H | H | CF₂CF₂CF₃ | H |
| 377 | A | — | Me | H | 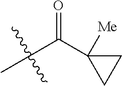 [SMILES: *C(=O)C1(C)CC1] | SEt | C—H | H | CF₃ | H |
| 378 | A | — | Me | H | 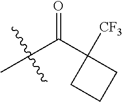 [SMILES: *C(=O)C1(C(F)(F)F)CCC1] | SEt | C—H | H | CF₃ | H |
| 379 | A | — | Me | H | 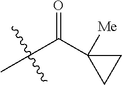 [SMILES: *C(=O)C1(C)CC1] | SO₂Et | C—H | H | CF₃ | H |
| 380 | A | — | Me | H | 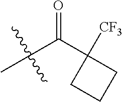 [SMILES: *C(=O)C1(C(F)(F)F)CCC1] | SO₂Et | C—H | H | CF₃ | H |
| 381 | A | — | Me | H | 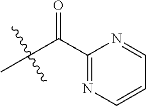 [SMILES: *C(=O)c1ncccn1] | SEt | C—H | H | CF₃ | H |
| 382 | A | — | c-Pr | H | —C(═O)CF₂CF₂H | SEt | C—H | H | CF₃ | H |
| 383 | A | — | c-Pr | H | —C(═O)CF₂CF₂H | SO₂Et | C—H | H | CF₃ | H |
| 384 | A | — | Me | H | —C(═O)CF₃ | SEt | C—H | H | CHCl₂ | H |
| 385 | A | — | Me | H | —C(═O)CF₂CF₂CF₃ | SEt | C—H | H | CF₂CF₂CF₃ | H |
| 386 | A | — | Me | H | —C(═O)CF₃ | SO₂Et | C—H | H | CHCl₂ | H |
| 387 | A | — | Me | H | —C(═O)CF₂CF₃ | SO₂Et | C—H | H | CF₂CF₂CF₃ | H |
| 388 | A | — | Me | H | 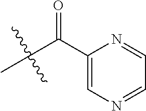 [SMILES: *C(=O)c1cnccn1] | SEt | C—H | H | CF₃ | H |
| 389 | A | — | Me | H | —C(═O)CF₂CF₂CF₃ | SO₂Et | C—H | H | CF₂CF₂CF₃ | H |
| 390 | A | — | Me | H | —C(═O)CF₃ | SEt | C—H | H | CClF₂ | H |
| 391 | A | — | Me | H | —C(═O)CF₃ | SO₂Et | C—H | H | CClF₂ | H |
| 392 | A | — | Me | H | H | SEt | C—H | H | CClF₂ | H |
| 393 | A | — | Me | H | H | SEt | C—H | H | CMe₂F | H |
| 394 | A | — | Me | H | 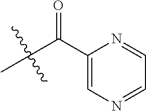 [SMILES: *C(=O)c1cnccn1] | SO₂Et | C—H | H | CF₃ | H |
| 395 | A | — | Me | H | 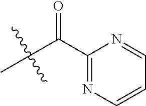 [SMILES: *C(=O)c1ncccn1] | SO₂Et | C—H | H | CF₃ | H |
| 396 | A | — | Me | H | —C(═O)CF₃ | SEt | C—H | H | CMe₂F | H |
| 397 | A | — | Me | H | —C(═O)CF₃ | SO₂Et | C—H | H | CMe₂F | H |
| 398 | A | — | Me | H | —C(═O)CH═CHOEt | SEt | C—H | H | CF₃ | H |
| 399 | A | — | Me | H | H | SO₂Et | C—H | H | CClF₂ | H |
| 400 | A | — | Me | H | —C(═O)CH═CHOEt | SO₂Et | C—H | H | CF₃ | H |
| 401 | A | — | Me | H | —C(═O)CH₂CH₂CF₃ | SEt | C—H | H | CF₃ | H |
| 402 | A | — | Me | H | —C(═O)CH₂CH₂CF₃ | SO₂Et | C—H | H | CF₃ | H |
| 403 | A | — | Et | H | —C(═O)CF₂Me | SEt | C—H | H | CF₃ | H |
| 404 | A | — | Et | H | —C(═O)CF₂CClF₂ | SEt | C—H | H | CF₃ | H |
| 405 | A | — | Et | H | —C(═O)CF₂Me | SO₂Et | C—H | H | CF₃ | H |
| 406 | A | — | Et | H | —C(═O)CF₂CClF₂ | SO₂Et | C—H | H | CF₃ | H |
| 407 | A | — | Et | H | —C(═O)CClF₂ | SO₂Et | C—H | H | CF₃ | H |
| 408 | A | — | Me | H | —C(═O)CCl₂F | SEt | C—H | H | CF₂CF₃ | H |
| 409 | A | — | Me | H | —C(═O)CClF₂ | SEt | C—H | H | CF₂CF₃ | H |
| 410 | A | — | Me | H | —C(═O)CCl₂F | SO₂Et | C—H | H | CF₂CF₃ | H |
| 411 | A | — | Me | H | —C(═O)CHCl₂ | SEt | C—H | H | CF₂CF₃ | H |
| 412 | A | — | Me | H | —C(═O)CF₂CF₂CF₂CF₃ | SEt | C—H | H | CF₂CF₃ | H |
| 413 | A | — | Me | H | —C(═O)CClF₂ | SO₂Et | C—H | H | CF₂CF₃ | H |
| 414 | A | — | Me | H | —C(═O)CF₂H | SEt | C—H | H | CF₂CF₃ | H |
| 415 | A | — | Me | H | —C(═O)CF₂CClF₂ | SEt | C—H | H | CF₂CF₃ | H |
| 416 | A | — | Me | H | —C(═O)CF₂H | SO₂Et | C—H | H | CF₂CF₃ | H |
| 417 | A | — | Me | H | —C(═O)CF₂CClF₂ | SO₂Et | C—H | H | CF₂CF₃ | H |
| 418 | A | — | Me | H | —C(═O)CH₂CHCF₃CF₃ | SEt | C—H | H | CF₃ | H |
| 419 | A | — | Me | H | —C(═O)CF₂Me | SEt | C—H | H | CF₂CF₃ | H |
| 420 | A | — | Me | H | —C(═O)CHCl₂ | SO₂Et | C—H | H | CF₂CF₃ | H |
| 421 | A | — | Me | H | —C(═O)CF₂CF₂CF₂CF₃ | SO₂Et | C—H | H | CF₂CF₃ | H |
| 422 | A | — | Me | H | —C(═O)CF₂Me | SO₂Et | C—H | H | CF₂CF₃ | H |
| 423 | A | — | Et | H | —C(═O)CHCl₂ | SEt | C—H | H | CF₃ | H |
| 424 | A | — | Et | H | —C(═O)CHCl₂ | SO₂Et | C—H | H | CF₃ | H |
| 425 | A | — | Et | H | —C(═O)CF₂CF₂CF₂CF₃ | SEt | C—H | H | CF₃ | H |
| 426 | A | — | Et | H | —C(═O)CF₂CF₂CF₂CF₃ | SO₂Et | C—H | H | CF₃ | H |
| 427 | A | — | Me | H | —C(═O)—i-Pent | SEt | C—H | H | CF₃ | H |
| 428 | A | — | Me | H | —C(═O)CH₂—c-Pr | SEt | C—H | H | CF₃ | H |
| 429 | A | — | Me | H | 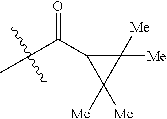 [SMILES: *C(=O)C1C(C)(C)C1(C)C] | SEt | C—H | H | CF₃ | H |
| 430 | A | — | Me | H | —C(═O)—i-Pent | SO₂Et | C—H | H | CF₃ | H |
| 431 | A | — | Me | H | —C(═O)CH₂—c-Pr | SO₂Et | C—H | H | CF₃ | H |
| 432 | A | — | Me | H | 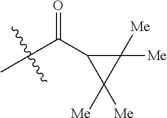 [SMILES: *C(=O)C1C(C)(C)C1(C)C] | SO₂Et | C—H | H | CF₃ | H |
| 433 | A | — | Me | H | —C(═O)Me | SO₂Et | C—H | H | CF₂CF₃ | H |
| 434 | A | — | Me | H | —C(═O)CMe₂CF₃ | SEt | C—H | H | CF₃ | H |
| 435 | A | — | Me | H | —C(═O)CH₂CHCF₃CF₃ | SO₂Et | C—H | H | CF₃ | H |
| 436 | A | — | Me | H | —C(═O)CMe₂CF₃ | SO₂Et | C—H | H | CF₃ | H |

Next, ¹H-NMR data of the compounds listed in Table 7 are shown in Table 8.

TABLE 8

| Compound | |
|---|---|
| No. | ¹H-NMR |
| 1 | ¹H-NMR (ACETONE-D₆) δ: 10.58 (1H, br s), 9.57 (1H, br |
| | s), 8.59 (1H, br s), 8.09-8.09 (1H, m), 3.94 (3H, s), 3.76 |
| | (2H, q, J = 7.4 Hz), 1.23 (3H, t, J = 7.4 Hz). |
| 2 | ¹H-NMR (DMSO-D₆) δ: 11.19 (1H, s), 9.65 (1H, d, J = 0.7 |
| | Hz), 8.61 (1H, s), 8.29 (1H, s), 6.56 (1H, t, J = 53.0 Hz), |
| | 3.79 (3H, s), 3.64 (2H, q, J = 7.4 Hz), 1.17 (3H, t, J = 7.4 |
| | Hz). |
| 3 | ¹H-NMR (DMSO-D₆) δ: 10.17 (1H, s), 9.61 (1H, s), 8.55 |
| | (1H, s), 8.25 (1H, s), 3.71 (3H, s), 3.57 (2H, q, J = 7.5 Hz), |
| | 2.09 (3H, s), 1.13 (3H, t, J = 7.5 Hz). |
| 4 | ¹H-NMR (CDCl₃) δ: 9.55 (1H, d, J = 0.7 Hz), 8.10 (1H, d, |
| | J = 1.0 Hz), 6.39 (1H, br s), 3.96 (3H, s), 2.84 (2H, q, J = |
| | 7.4 Hz), 1.53 (9H, s), 1.16 (3H, t, J = 7.4 Hz). |
| 5 | ¹H-NMR (CDCl₃) δ: 9.54 (1H, s), 8.08 (1H, d, J = 1.0 Hz), |
| | 4.09 (2H, br s), 3.85 (3H, s), 2.80 (2H, q, J = 7.3 Hz), 1.19 |
| | (3H, t, J = 7.3 Hz). |
| 6 | ¹H-NMR (CDCl₃) δ: 9.56 (1H, d, J = 0.7 Hz), 8.13 (1H, d, |
| | J = 0.7 Hz), 8.02 (1H, br s), 3.98 (3H, s), 2.89 (2H, q, J = |
| | 7.3 Hz), 1.16 (3H, t, J = 7.3 Hz). |
| 7 | ¹H-NMR (CDCl₃) δ: 9.56 (1H, s), 8.12 (1H, s), 8.00 (1H, |
| | br s), 6.16 (1H, t, J = 53.7 Hz), 3.97 (3H, s), 2.88 (2H, q, |
| | J = 7.3 Hz), 1.16 (3H, t, J = 7.3 Hz). |
| 8 | ¹H-NMR (DMSO-D₆) δ: 10.09 (1H, s), 8.69 (1H, s), 3.88 |
| | (3H, br s), 3.79-3.77 (2H, br m), 1.23 (3H, t, J = 7.4 Hz). |
| 9 | ¹H-NMR (DMSO-D₆) δ: 10.09 (1H, s), 8.69 (1H, s), 6.56 |
| | (1H, t, J = 52.9 Hz), 3.84 (3H, s), 3.76 (2H, q, J = 7.5 Hz), |
| | 1.22 (3H, t, J = 7.5 Hz). |
| 10 | ¹H-NMR (CDCl₃) δ: 9.55 (1H, s), 8.11 (1H, s), 7.21 (1H, |
| | br s), 3.94 (3H, s), 2.85 (2H, q, J = 7.3 Hz), 2.32 (3H, s), |
| | 1.16 (3H, t, J = 7.3 Hz). |
| 11 | ¹H-NMR (DMSO-D₆) δ: 10.29 (1H, br s), 10.08 (1H, s), |
| | 8.68 (1H, s), 3.78 (3H, s), 3.71 (2H, q, J = 7.5 Hz), 2.12 |
| | (3H, s), 1.22 (3H, t, J = 7.5 Hz). |
| 12 | ¹H-NMR (CDCl₃) δ: 9.56 (1H, s), 9.53 (1H, s), 8.16 (1H, |
| | s), 8.13 (1H, s), 4.07-4.04 (1H, m), 3.95 (3H, s), 3.88 (3H, |
| | s), 3.84-3.82 (1H, m), 3.74-3.71 (2H, m), 3.40 (3H, s), |
| | 3.27 (3H, s), 2.35 (3H, s), 1.97 (3H, s), 1.43 (3H, t, J = 7.5 |
| | Hz), 1.39 (3H, t, J = 7.5 Hz). |
| 13 | ¹H-NMR (CDCl₃) δ: 9.55 (1H, s), 8.11 (1H, s), 3.97 (3H, |
| | s), 3.85 (3H, s), 2.86 (2H, q, J = 7.3 Hz), 1.16 (3H, t, J = |
| | 7.3 Hz). |
| 14 | ¹H-NMR (DMSO-D₆) δ: 10.08 (1H, d, J = 0.9 Hz), 9.88 |
| | (1H, br s), 8.68 (1H, d, J = 0.9 Hz), 3.82 (3H, s), 3.74 (2H, |
| | q, J = 7.3 Hz), 3.71 (3H, br s), 1.25 (3H, t, J = 7.3 Hz ). |
| 15 | ¹H-NMR (CDCl₃) δ: 9.15 (1H, s), 8.63 (1H, s), 8.17 (1H, |
| | br s), 7.96 (1H, s), 3.91 (3H, s), 2.65 (2H, q, J = 7.3 Hz), |
| | 1.13 (3H, t, J = 7.3 Hz). |
| 16 | ¹H-NMR (CDCl₃) δ: 9.14 (1H, s), 8.62 (1H, s), 8.20 (1H, |
| | br s), 7.95 (1H, s), 3.90 (3H, s), 2.65 (2H, q, J = 7.3 Hz), |
| | 1.13 (3H, t, J = 7.3 Hz). |
| 17 | ¹H-NMR (DMSO-D₆) δ: 12.03 (1H, br s), 9.64 (1H, d, J = |
| | 0.6 Hz), 8.61 (1H, s), 8.29 (1H, s), 3.80 (3H, s), 3.67 (2H, |
| | q, J = 7.3 Hz), 1.15 (3H, t, J = 7.3 Hz). |
| 18 | ¹H-NMR (DMSO-D₆) δ: 12.04 (1H, br s), 9.64 (1H, d, J = |
| | 0.7 Hz), 8.61 (1H, s), 8.29 (1H, s), 3.79 (3H, s), 3.67 (2H, |
| | q, J = 7.2 Hz), 1.16 (3H, t, J = 7.2 Hz). |
| 19 | ¹H-NMR (CDCl₃) δ: 9.56 (1H, s), 8.15 (1H, s), 8.13 (1H, |
| | d, J = 1.2 Hz), 3.96 (3H, s), 2.88 (2H, q, J = 7.3 Hz), 1.15 |
| | (3H, t, J = 7.3 Hz). |
| 20 | ¹H-NMR (CDCl₃) δ: 9.56 (1H, d, J = 0.6 Hz), 8.13-8.12 |
| | (2H, m), 3.96 (3H, s), 2.89 (2H, q, J = 7.3 Hz), 1.15 (3H, t, |
| | J = 7.3 Hz). |
| 21 | ¹H-NMR (CDCl₃) δ: 9.55 (1H, d, J = 0.8 Hz), 8.11 (1H, d, |
| | J = 0.8 Hz), 7.20 (1H, s), 3.94 (3H, s), 2.84 (2H, q, J = 7.3 |
| | Hz), 2.55 (2H, q, J = 7.4 Hz), 1.32 (3H, t, J = 7.4 Hz), 1.15 |
| | (3H, t, J = 7.3 Hz). |
| 22 | ¹H-NMR (CDCl₃) δ: 9.55 (1H, d, J = 1.0 Hz), 8.11 (1H, d, |
| | J = 1.0 Hz), 7.20 (1H, s), 3.93 (3H, s), 2.84 (2H, q, J = 7.3 |
| | Hz), 2.50 (2H, t, J = 7.3 Hz), 1.89-1.78 (2H, m), 1.15 (3H, |
| | t, J = 7.3 Hz), 1.08 (3H, t, J = 7.3 Hz). |
| 23 | ¹H-NMR (DMSO-D₆) δ: 12.15 (1H, br s), 10.09 (1H, s), |
| | 8.70 (1H, s), 3.86 (3H, s), 3.81-3.75 (2H, m), 1.22 (3H, t, |
| | J = 7.5 Hz). |
| 24 | ¹H-NMR (CD3OD) δ: 9.84 (1H, s), 8.38 (1H, s), 3.89 (3H, |
| | s), 3.85 (2H, q, J = 7.5 Hz), 1.34 (3H, t, J = 7.5 Hz). |
| 25 | ¹H-NMR (CD3OD) δ: 9.73 (1H, s), 8.27 (1H, s), 3.76 (3H, |
| | s), 3.71 (2H, q, J = 7.5 Hz), 2.39 (2H, t, J = 7.3 Hz), 1.73- |
| | 1.63 (2H, m), 1.23 (3H, t, J = 7.5 Hz), 0.96 (3H, t, J = 7.5 |
| | Hz). |
| 26 | ¹H-NMR (CDCl₃) δ: 9.13 (1H, s), 8.65 (1H, s), 7.96 (1H, |
| | s), 6.30 (1H, br s), 3.92 (3H, s), 2.64 (2H, q, J = 7.3 Hz), |
| | 1.52 (9H, s), 1.16 (3H, t, J = 7.3 Hz). |
| 27 | ¹H-NMR (CDCl₃) δ: 9.15 (1H, s), 8.63 (1H, s), 7.99-7.97 |
| | (2H, m), 3.93 (3H, s), 2.66 (2H, q, J = 7.2 Hz), 1.14 (3H, t, |
| | J = 7.2 Hz). |
| 28 | ¹H-NMR (CDCl₃) δ: 9.14 (1H, s), 8.68 (1H, s), 7.93 (1H, |
| | s), 3.81 (3H, s), 3.29 (3H, s), 2.68 (2H, q, J = 7.3 Hz), 1.40 |
| | (9H, s), 1.16 (3H, t, J = 7.3 Hz). |
| 29 | ¹H-NMR (CDCl₃) δ: 9.12 (1H, s), 8.65 (1H, s), 7.92 (1H, |
| | s), 3.89 (3H, s), 2.96 (3H, s), 2.58 (2H, q, J = 7.3 Hz), 1.17 |
| | (3H, t, J = 7.3 Hz). |
| 30 | ¹H-NMR (CDCl₃) δ: 9.15 (1H, mixture, s), 8.64 (1H, |
| | minor, s), 8.62 (1H, major, s), 7.97 (1H, mixture, s), 3.88 |
| | (3H, major, s), 3.83 (3H, minor, s), 3.58 (3H, minor, s), |
| | 3.39 (3H, major, s), 2.79-2.76 (1H, mixture, m), 2.70-2.66 |
| | (1H, mixture, m), 1.21 (3H, major, t, J = 7.5 Hz), 1.15 |
| | (3H, minor, t, J = 7.3 Hz). |
| 31 | ¹H-NMR (CDCl₃) δ: 9.12 (1H, s), 8.56 (1H, s), 7.93 (1H, |
| | s), 5.72-5.70 (1H, br m), 3.94 (3H, s), 3.26 (2H, q, J = 7.3 |
| | Hz), 3.01 (3H, d, J = 3.7 Hz), 1.26 (3H, t, J = 7.3 Hz). |
| 32 | ¹H-NMR (CDCl₃) δ: 9.17-9.16 (1H, mixture, m), 8.60 (1H, |
| | minor, s), 8.52 (1H, major, s), 7.97 (1H, mixture, s), 3.92 |
| | (3H, major, s), 3.89 (3H, minor, s), 3.61-3.59 (2H, minor, |
| | br m), 3.57 (3H, minor, s), 3.40 (3H, major, s), 3.30-3.28 |
| | (2H, major, br m), 1.27-1.25 (3H, mixture, m). |
| 33 | ¹H-NMR (CDCl₃) δ: 9.16-9.15 (1H, mixture, m), 8.65 (1H, |
| | minor, s), 8.62 (1H, major, s), 7.97 (1H, mixture, s), 3.88 |
| | (3H, major, s), 3.82 (3H, minor, s), 3.61 (3H, minor, s), |
| | 3.38 (3H, major, s), 2.79-2.75 (2H, mixture, m), 2.70-2.66 |
| | (2H, mixture, m), 1.21 (3H, major, t, J = 7.3 Hz), 1.14 |
| | (3H, minor, t, J = 7.3 Hz). |
| 34 | ¹H-NMR (CDCl₃) δ: 9.17 (1H, major, s), 9.15 (1H, minor, |
| | s), 8.60 (1H, minor, s), 8.52 (1H, major, s), 7.97 (1H, |
| | mixture, s), 3.92 (3H, major, s), 3.88 (3H, minor, s), 3.60 |
| | (3H, minor, t, J = 2.5 Hz), 3.59-3.57 (1H, major, m), 3.40- |
| | 3.37 (1H, minor, m), 3.39 (3H, major, s), 3.32-3.30 (1H, |
| | major, m), 3.26-3.24 (1H, minor, m), 1.36 (3H, major, t, |
| | J = 7.5 Hz), 1.24 (3H, minor, t, J = 7.5 Hz). |
| 35 | ¹H-NMR (CDCl₃) δ: 9.15 (1H, mixture, s), 8.65 (1H, |
| | minor, s), 8.62 (1H, major, s), 7.96 (1H, mixture, s), 3.87 |
| | (3H, major, s), 3.82 (3H, minor, s), 3.59 (3H, minor, s), |
| | 3.38 (3H, major, s), 2.81-2.74 (1H, mixture, m), 2.69-2.66 |
| | (1H, mixture, m), 1.21 (3H, major, t, J = 7.5 Hz), 1.15 |
| | (3H, minor, t, J = 7.3 Hz). |
| 36 | ¹H-NMR (CDCl₃) δ: 9.17 (1H, major, s), 9.15 (1H, minor, |
| | s), 8.60 (1H, minor, s), 8.52 (1H, major, s), 7.97 (1H, |
| | mixture, s), 3.91 (3H, major, s), 3.87 (3H, minor, s), 3.60 |
| | (3H , minor, t, J = 2.6 Hz), 3.57-3.55 (1H, major, , m), |
| | 3.39-3.37 (1H, minor, m), 3.39 (3H, major, s), 3.33-3.24 |
| | (1H, mixture, m), 1.35 (3H, major, t, J = 7.2 Hz), 1.25 |
| | (3H, minor, t, J = 7.2 Hz). |
| 37 | ¹H-NMR (CDCl₃) δ: 9.16 (1H, major, s), 9.15 (1H, minor, |
| | s), 8.65 (1H, minor, s), 8.61 (1H, major, s), 7.96 (1H, |
| | mixture, s), 6.28 (1H, minor, t, J = 53.2 Hz), 5.91 (1H, |
| | major, t, J = 53.2 Hz), 3.89 (3H, major, s), 3.82 (3H, |
| | minor, s), 3.58 (3H, minor, s), 3.35 (3H, major, s), 2.79- |
| | 2.67 (2H, mixture, m), 1.21 (3H, major, t, J = 7.3 Hz), |
| | 1.15 (3H, minor, t, J = 7.5 Hz). |
| 38 | ¹H-NMR (CDCl₃) δ: 9.17 (1H, major, s), 9.16 (1H, minor, |
| | s), 8.60 (1H, minor, s), 8.50 (1H, major, s), 7.97 (1H, |
| | mixture, s),6.29 (1H, minor, t, J = 53.0 Hz), 6.14 (1H, |
| | major, t, J = 53.5 Hz), 3.92 (3H, major, s), 3.88 (3H, |
| | minor, s), 3.63-3.57 (1H, major, m), 3.55 (3H, minor, s), |
| | 3.39-3.36 (1H, mixture, m), 3.34 (3H, major, s), 3.28-3.25 |
| | (1H, minor, m), 1.34 (3H, major, t, J = 7.3 Hz), 1.26 (3H, |
| | minor, t, J = 7.3 Hz). |
| 39 | ¹H-NMR (CDCl₃) δ: 9.12 (1H, s), 8.66 (1H, s), 7.92 (1H, |
| | s), 4.03 (2H, br s), 3.81 (3H, s), 2.61 (2H, q, J = 7.4 Hz), |
| | 1.18 (3H, t, J = 7.4 Hz). |
| 40 | ¹H-NMR (CDCl₃) δ: 9.14 (1H, s), 8.64 (1H, s), 7.96 (1H, |
| | s), 6.48 (1H, br s), 3.93 (3H, s), 3.84 (3H, s), 2.64 (2H, q, |
| | J = 7.3 Hz), 1.15 (3H, t, J = 7.3 Hz). |
| 41 | ¹H-NMR (CDCl₃) δ: 9.13 (1H, s), 8.62 (1H, s), 7.96 (1H, |
| | s), 7.13 (1H, br s), 3.89 (3H, s), 2.63 (2H, q, J = 7.2 Hz), |
| | 2.55-2.53 (2H, br m), 1.32 (3H, t, J = 7.3 Hz), 1.14 (3H, t, |
| | J = 7.2 Hz). |
| 42 | ¹H-NMR (CDCl₃) δ: 9.13 (1H, s), 8.52 (1H, s), 8.26 (1H, |
| | br s), 7.95 (1H, s), 3.89 (3H, s), 3.37 (2H, q, J = 7.4 Hz), |
| | 2.55 (2H, q, J = 7.6 Hz), 1.30 (3H, t, J = 7.6 Hz), 1.24 (3H, |
| | t, J = 7.4 Hz). |
| 43 | ¹H-NMR (CDCl₃) δ: 9.13 (1H, s), 8.53 (1H, s), 7.95 (1H, |
| | s), 7.69 (1H, br s), 3.94 (3H, s), 3.86 (3H, s), 3.36 (2H, q, |
| | J = 7.4 Hz), 1.26 (3H, t, J = 7.4 Hz). |
| 44 | ¹H-NMR (CDCl₃) δ: 9.14 (1H, s), 8.54 (1H, s), 7.95 (1H, |
| | s), 7.65 (1H, br s), 4.29 (2H, q, J = 7.2 Hz), 3.94 (3H, s), |
| | 3.35 (2H, q, J = 7.2 Hz), 1.35 (3H, t, J = 7.2 Hz), 1.26 (3H, |
| | t, J = 7.2 Hz). |
| 45 | ¹H-NMR (CDCl₃) δ: 9.13 (1H, s), 8.54 (1H, s), 7.95 (1H, |
| | s), 7.66 (1H, br s), 4.19 (2H, t, J = 6.7 Hz), 3.94 (3H, s), |
| | 3.35 (2H, q, J = 7.3 Hz), 1.75-1.73 (2H, m). 1.26 (3H, t, |
| | J = 7.2 Hz), 1.00 (3H, t, J = 7.3 Hz). |
| 46 | ¹H-NMR (CDCl₃) δ: 9.56 (1H, d, J = 0.9 Hz), 8.17 (1H, br |
| | s), 8.13 (1H, d, J = 0.9 Hz), 3.96 (3H, s), 2.89 (2H, q, J = |
| | 7.3 Hz), 1.15 (3H, t, J = 7.3 Hz). |
| 47 | ¹H-NMR (CDCl₃) δ: 9.13 (1H, s), 8.63 (1H, s), 7.96 (1H, |
| | s), 7.14 (1H, br s), 3.89 (3H, s), 2.63 (2H, q, J = 7.4 Hz), |
| | 2.48 (2H, t, J = 7.2 Hz), 1.84-1.81 (2H, m), 1.14 (3H, t, J = |
| | 7.4 Hz), 1.08 (3H, t, J = 7.2 Hz). |
| 48 | ¹H-NMR (CDCl₃) δ: 9.13 (1H, s), 8.52 (1H, s), 8.26 (1H, |
| | br s), 7.95 (1H, s), 3.89 (3H, s), 3.37 (2H, q, J = 7.4 Hz), |
| | 2.49 (2H, t, J = 7.4 Hz), 1.82-1.78 (2H, m), 1.24 (3H, t, J = |
| | 7.4 Hz), 1.05 (3H, t, J = 7.4 Hz). |
| 49 | ¹H-NMR (CDCl₃) δ: 9.13 (1H, s), 8.63 (1H, s), 7.96 (1H, |
| | s), 7.16 (1H, br s), 3.87 (3H, s), 2.72-2.68 (1H, m), 2.62 |
| | (2H, q, J = 7.3 Hz), 1.34 (6H, d, J = 6.8 Hz), 1.14 (3H, t, |
| | J = 7.3 Hz). |
| 50 | ¹H-NMR (CDCl₃) δ: 9.13 (1H, s), 8.63 (1H, s), 7.97 (1H, |
| | s), 7.12 (1H, br s), 3.89 (3H, s), 2.63 (2H, q, J = 7.2 Hz), |
| | 2.50 (2H, t, J = 7.6 Hz), 1.81-1.73 (2H, m), 1.48-1.46 |
| | (2H, m), 1.15 (3H, t, J = 7.2 Hz), 0.99 (3H, t, J = 7.1 Hz). |
| 51 | ¹H-NMR (CDCl₃) δ: 9.13 (1H, s), 8.52 (1H, s), 8.31 (1H, |
| | br s), 7.95 (1H, s), 3.87 (3H, s), 3.36 (2H, q, J = 7.3 Hz), |
| | 2.72-2.70 (1H, m), 1.32 (6H, d, J = 7.0 Hz), 1.23 (3H, t, |
| | J = 7.3 Hz). |
| 52 | ¹H-NMR (CDCl₃) δ: 9.13 (1H, s), 8.52 (1H, s), 8.26 (1H, |
| | br s), 7.95 (1H, s), 3.89 (3H, s), 3.37 (2H, q, J = 7.2 Hz), |
| | 2.51 (2H, t, J = 7.5 Hz), 1.78-1.72 (2H, m), 1.46-1.43 |
| | (2H, m), 1.24 (3H, t, J = 7.2 Hz), 0.98 (3H, t, J = 7.5 Hz). |
| 53 | ¹H-NMR (DMSO-D₆) δ: 12.07 (1H, br s), 9.52 (1H, d, J = |
| | 0.7 Hz), 8.40 (1H, d, J = 0.7 Hz), 3.83 (3H, s), 3.76 (2H, q, |
| | J = 7.4 Hz), 1.19 (3H, t, J = 7.4 Hz). |
| 54 | ¹H-NMR (DMSO-D₆) δ: 12.07 (1H, br s), 9.48 (1H, s), |
| | 8.38 (1H, s), 3.82 (3H, s), 3.73 (2H, q, J = 7.4 Hz), 1.19 |
| | (3H, t, J = 7.4 Hz). |
| 55 | ¹H-NMR (CDCl₃) δ: 9.14 (1H, s), 8.63 (1H, s), 8.13 (1H, |
| | br s), 7.96 (1H, s), 3.91 (3H, s), 2.66 (2H, q, J = 7.3 Hz), |
| | 1.14 (3H, t, J = 7.3 Hz). |
| 56 | ¹H-NMR (DMSO-D₆) δ: 12.05 (1H, br s), 9.64 (1H, s), |
| | 8.61 (1H, s), 8.29 (1H, s), 3.78 (3H, s), 3.68-3.66 (2H, br |
| | m), 1.15 (3H, t, J = 7.3 Hz). |
| 57 | ¹H-NMR (DMSO-D₆) δ: 12.16 (1H, br s), 10.09 (1H, s), |
| | 8.70 (1H, s), 3.84 (3H, s), 3.78 (2H, q, J = 7.5 Hz), 1.22 |
| | (3H, t, J = 7.5 Hz). |
| 58 | ¹H-NMR (CDCl₃) δ: 9.15 (1H, s), 8.64 (1H, s), 8.13 (1H, |
| | br s), 7.96 (1H, s), 6.24 (1H, tt, J = 52.7, 5.0 Hz), 3.90 |
| | (3H, s), 2.65 (2H, q, J = 7.0 Hz), 1.14 (3H, t, J = 7.0 Hz). |
| 59 | ¹H-NMR (CDCl₃) δ: 9.31 (1H, br s), 9.15 (1H, s), 8.51 |
| | (1H, s), 7.96 (1H, s), 6.20 (1H, tt, J = 52.7, 4.8 Hz), 3.93 |
| | (3H, s), 3.44 (2H, q, J = 7.4 Hz), 1.25 (3H, t, J = 7.4 Hz). |
| 60 | ¹H-NMR (CDCl₃) δ: 9.12 (1H, s), 8.66 (1H, s), 7.92 (1H, |
| | s), 4.13 (2H, q, J = 7.3 Hz), 4.02 (2H, br s), 2.60 (2H, q, |
| | J = 7.3 Hz), 1.48 (3H, t, J = 7.3 Hz), 1.18 (3H, t, J = 7.3 |
| | Hz). |
| 61 | ¹H-NMR (CDCl₃) δ: 9.14 (1H, d, J = 0.6 Hz), 8.65 (1H, s), |
| | 7.97 (1H, s), 7.88 (1H, br s), 4.19 (2H, q, J = 7.3 Hz), 2.67 |
| | (2H, q, J = 7.3 Hz), 1.55 (11H, t, J = 7.3 Hz), 1.15 (3H, t, |
| | J = 7.3 Hz). |
| 62 | ¹H-NMR (CDCl₃) δ: 9.14 (1H, d, J = 0.6 Hz), 8.65 (1H, s), |
| | 8.03 (1H, br s), 7.97 (1H, s), 4.18 (2H, q, J = 7.3 Hz), 2.66 |
| | (2H, q, J = 7.3 Hz), 1.54 (3H, t, J = 7.3 Hz), 1.14 (3H, t, |
| | J = 7.3 Hz). |
| 63 | ¹H-NMR (CDCl₃) δ: 9.14 (1H, s), 8.65 (1H, s), 8.00 (1H, |
| | br s), 7.97 (1H, s), 4.18 (2H, q, J = 7.3 Hz), 2.66 (2H, q, |
| | J = 7.3 Hz), 1.53 (3H, t, J = 7.3 Hz), 1.14 (3H, t, J = 7.3 |
| | Hz). |
| 64 | ¹H-NMR (DMSO-D₆) δ: 11.90 (1H, br s), 9.64 (1H, d, J = |
| | 0.6 Hz), 8.62 (1H, s), 8.30 (1H, s), 4.14 (2H, q, J = 7.3 |
| | Hz), 3.64 (2H, q, J = 7.3 Hz), 1.37 (3H, t, J = 7, 3 Hz), |
| | 1.16 (3H, t, J = 7.3 Hz). |
| 65 | ¹H-NMR (DMSO-D₆) δ: 12.02 (1H, s), 9.64 (1H, s), 8.62 |
| | (1H, s), 8.30 (1H, s), 4.11 (2H, q, J = 7.3 Hz), 3.65 (2H, q, |
| | J = 7.3 Hz), 1.36 (3H, t, J = 7.3 Hz), 1.15 (3H, t, J = 7.3 |
| | Hz). |
| 66 | ¹H-NMR (DMSO-D₆) δ: 12.03 (1H, br s), 9.64 (1H, s), |
| | 8.62 (1H, s), 8.30 (1H, s), 4.09 (2H, q, J = 7.3 Hz), 3.65 |
| | (2H, q, J = 7.3 Hz), 1.36 (3H, t, J = 7.3 Hz), 1.16 (3H, t, |
| | J = 7.3 Hz). |
| 67 | ¹H-NMR (CDCl₃) δ: 9.13 (1H, s), 8.64 (1H, s), 7.95 (1H, |
| | s), 7.34 (1H, br s), 3.88 (3H, s), 2.65-2.63 (2H, br m), |
| | 1.71-1.69 (1H, br m), 1.16-1.14 (5H, m), 0.99-0.97 (2H, br |
| | m). |
| 68 | ¹H-NMR (CDCl₃) δ: 9.13 (1H, s), 8.53 (1H, s), 8.48 (1H, |
| | br s), 7.95 (1H, s), 3.87 (3H, s), 3.36 (2H, q, J = 7.3 Hz), |
| | 1.75-1.72 (1H, m), 1.24 (3H, t, J = 7.3 Hz), 1.15-1.12 (2H, |
| | m), 1.03-1.01(2H, m). |
| 69 | ¹H-NMR (CDCl₃) δ: 9.71 (1H, br s), 9.16 (1H, s), 8.67 |
| | (1H, s), 7.94 (1H, s), 3.85 (3H, s), 3.40 (2H, q, J = 10.3 |
| | Hz), 2.66 (2H, q, J = 7.3 Hz), 1.14 (3H, t, J = 7.3 Hz). |
| 70 | ¹H-NMR (DMSO-D₆) δ: 10.66 (1H, br s), 9.63 (1H, s), |
| | 8.58 (1H, s), 8.27 (1H, s), 3.73 (3H, s), 3.67 (2H, q, J = |
| | 10.8 Hz), 3.60 (2H, q, J = 7.3 Hz), 1.14 (3H, t, J = 7.3 Hz). |
| 71 | ¹H-NMR (CDCl₃) δ: 9.13 (1H, s), 8.62 (1H, s), 7.94 (1H, |
| | s), 7.12 (1H, br s), 3.89 (3H, s), 2.63 (2H, q, J = 7.4 Hz), |
| | 2.37 (2H, d, J = 6.8 Hz), 2.30-2.23 (1H, m), 1.15 (3H, t, |
| | J = 7.3 Hz), 1.09 (6H, d, J = 6.6 Hz). |
| 72 | ¹H-NMR (CDCl₃) δ: 9.13 (1H, s), 8.51 (1H, s), 8.26 (1H, |
| | br s), 7.95 (1H, s), 3.89 (3H, s), 3.37 (2H, q, J = 7.3 Hz), |
| | 2.38 (2H, d, J = 7.3 Hz), 2.28-2.22 (1H, m), 1.25 (3H, t, |
| | J = 7.3 Hz), 1.06 (6H, d, J = 6.7 Hz). |
| 73 | ¹H-NMR (CDCl₃) δ: 9.16 (1H, br s), 9.13 (1H, d, J = 0.7 |
| | Hz), 8.52 (1H, s), 7.95 (1H, s), 4.27 (2H, s), 3.92 (3H, s), |
| | 3.40 (2H, q, J = 7.3 Hz), 1.26 (3H, t, J = 7.3 Hz). |
| 74 | ¹H-NMR (CDCl₃) δ: 9.11 (1H, s), 8.79 (1H, br s), 8.52 |
| | (1H, s), 7.94 (1H, s), 7.74 (1H, dd, J = 2.0, 0.5 Hz), 7.60 |
| | (1H, dd, J = 2.4, 0.5 Hz), 6.45 (1H, dd, J = 2.4, 2.0 Hz), |
| | 5.07 (2H, s), 3.87 (3H, s), 3.31 (2H, q, J = 7.4 Hz), 1.21 |
| | (3H, t, J = 7.4 Hz). |
| 75 | ¹H-NMR (CDCl₃) δ: 10.27 (1H, br s), 9.21 (1H, s), 8.59 |
| | (1H, s), 8.33 (1H, s), 8.02 (1H, s), 7.94 (1H, s), 5.20 (2H, |
| | s), 3.86 (3H, s), 3.35 (2H, q, J = 7.5 Hz), 1.23 (2H, t, J = |
| | 7.5 Hz). |
| 76 | ¹H-NMR (CDCl₃) δ: 9.13 (1H, s), 8.64 (1H, s), 7.96 (1H, |
| | s), 7.09 (1H, s), 4.18 (2H, q, J = 7.2 Hz), 2.64 (2H, q, J = |
| | 7.2 Hz), 2.30 (3H, s), 1.53 (3H, t, J = 7.2 Hz), 1.15 (3 H, t, |
| | J = 7.2 Hz). |
| 77 | ¹H-NMR (CDCl₃) δ: 9.13 (1H, d, J = 0.6 Hz), 8.50 (1H, d, |
| | J = 0.6 Hz), 8.12 (1H, br s), 7.96 (1H, t, J = 0.6 Hz), 4.19 |
| | (2H, q, J = 7.3 Hz), 3.40 (2H, q, J = 7.3 Hz), 2.29 (3H, s), |
| | 1.55 (3H, t, J = 7.3 Hz), 1.25 (3H, t, J = 7.3 Hz). |
| 78 | ¹H-NMR (CDCl₃) δ: 9.13 (1H, s), 8.65 (1H, s), 7.96 (1H, |
| | s), 7.04 (1H, br s), 4.18 (2H, q, J = 7.3 Hz), 2.63 (2H, q, |
| | J = 7.3 Hz), 2.54 (2H, q, J = 7.3 Hz), 1.53 (4H, t, J = 7.3 |
| | Hz), 1.32 (3H, t, J = 7.3 Hz), 1.14 (3H, t, J = 7.3 Hz). |
| 79 | ¹H-NMR (CDCl₃) δ: 9.13 (1H, s), 8.65 (1H, s), 7.95 (1H, |
| | s), 7.05 (1H, br s), 4.18 (2H, q, J = 7.2 Hz), 2.63 (2H, q, |
| | J = 7.2 Hz), 2.48 (2H, t, J = 7.2 Hz), 1.85-1.81 (2H, m), |
| | 1.53 (3H, t, J = 7.2 Hz), 1.14 (3H, t, J = 7.2 Hz), 1.08 (3H, |
| | t, J = 7.2 Hz). |
| 80 | ¹H-NMR (CDCl₃) δ: 9.13 (1H, d, J = 0.7 Hz), 8.50 (1H, s), |
| | 8.14 (1H, s), 7.96 (1H, s), 4.18 (2H, q, J = 7.2 Hz), 3.39 |
| | (2H, q, J = 7.4 Hz), 2.54 (2H, q, J = 7.6 Hz), 1.55 (3H, t, |
| | J = 7.2 Hz), 1.29 (3H, t, J = 7.6 Hz), 1.24 (3H, t, J = 7.4 |
| | Hz). |
| 81 | ¹H-NMR (CDCl₃) δ: 9.13 (1H, d, J = 0.7 Hz), 8.50 (1H, s), |
| | 8.14 (1H, s), 7.96 (1H, s), 4.18 (2H, q, J = 7.3 Hz), 3.39 |
| | (2H, q, J = 7.3 Hz), 2.48 (2H, t, J = 7.4 Hz), 1.85-1.75 |
| | (2H, m), 1.55 (3H, t, J = 7.3 Hz), 1.24 (3H, t, J = 7.3 Hz), |
| | 1.05 (3H, t, J = 7.3 Hz). |
| 82 | ¹H-NMR (CDCl₃) δ: 9.12 (1H, s), 8.58 (1H, s), 7.93 (1H, |
| | s), 5.08 (2H, s), 4.09 (2H, q, J = 7.3 Hz), 3.29 (2H, q, J = |
| | 7.3 Hz), 1.49 (3H, t, J = 7.3 Hz), 1.28 (3H, t, J = 7.3 Hz). |
| 83 | ¹H-NMR (CDCl₃) δ: 9.13 (1H, s), 8.65 (1H, s), 8.01-7.97 |
| | (2H, m), 7.95 (1H, s), 7.92 (1H, br s), 7.69-7.63 (1H, m), |
| | 7.60-7.54 (2H, m), 3.97 (3H, s), 2.63 (2H, q, J = 7.3 Hz), |
| | 1.13 (3H, t, J = 7.3 Hz). |
| 84 | ¹H-NMR (CDCl₃) δ: 9.91 (1H, s), 9.14 (1H, d, J = 0.9 Hz), |
| | 8.71 (1H, dq, J = 4.8, 0.9 Hz), 8.69 (1H, s), 8.29 (1H, dt, |
| | J = 7.6, 1.1 Hz), 7.99-7.95 (2H, m), 7.59 (1H, ddd, J = 7.6, |
| | 4.8, 1.1 Hz), 3.99 (3H, s), 2.64 (2H, q, J = 7.2 Hz), 1.14 |
| | (3H, t, J = 7.2 Hz). |
| 85 | ¹H-NMR (DMSO-D₆) δ: 10.56 (1H, s), 9.65 (1H, d, J = 0.7 |
| | Hz), 8.61 (1H, s), 8.29-8.27 (1H, m), 8.03-7.99 (2H, m), |
| | 7.69-7.64 (1H, m), 7.61-7.56 (2H, m), 3.81 (3H, s), 3.63 |
| | (2H, q, J = 7.4 Hz), 1.15 (3H, t, J = 7.4 Hz). |
| 86 | ¹H-NMR (DMSO-D₆) δ: 10.87 (1H, s), 9.64 (1H, d, J = 0.6 |
| | Hz), 8.81-8.78 (1H, m), 8.61 (1H, s), 8.28 (1H, t, J = 0.6 |
| | Hz), 8.17 (1H, dt, J = 7.7, 1.2 Hz), 8.10 (1H, td, J = 7.7, |
| | 1.7 Hz), 7.75 (1H, ddd, J = 7.7, 4.7, 1.2 Hz), 3.81 (3H, s), |
| | 3.67 (2H, q, J = 7.3 Hz), 1.16 (3H, t, J = 7.3 Hz). |
| 87 | ¹H-NMR (CDCl₃) δ: 9.25 (1H, br s), 9.14 (1H, s), 8.88 |
| | (1H, dd, J = 4.9, 1.5 Hz), 8.64 (1H, s), 8.32 (1H, d, J = 8.0 |
| | Hz), 8.13 (1H, br s), 7.94 (1H, s), 7.53 (1H, dd, J = 8.0, |
| | 4.9 Hz), 3.96 (3H, s), 2.64 (2H, q, J = 7.2 Hz), 1.13 (3H, t, |
| | J = 7.2 Hz). |
| 88 | ¹H-NMR (CDCl₃) δ: 9.14 (1H, s), 8.69 (1H, s), 7.95 (1H, |
| | s), 4.14-4.08 (2H, m), 3.26 (3H, s), 2.74-2.66 (2H, m), |
| | 1.52 (3H, t, J = 7.2 Hz), 1.39 (9H, s), 1.17 (3H, t, J = 7.3 |
| | Hz). |
| 89 | ¹H-NMR (CDCl₃) δ: 9.12 (1H, s), 8.66 (1H, s), 7.93 (1H, |
| | s), 4.21 (2H, q, J = 7.3 Hz), 3.86 (1H, br s), 2.96 (3H, s), |
| | 2.59 (2H, q, J = 7.3 Hz), 1.52 (4H, t, J = 7.3 Hz), 1.16 |
| | (3H, t, J = 7.3 Hz). |
| 90 | ¹H-NMR (CDCl₃) δ: 9.12 (1H, s), 8.55 (1H, s), 7.94 (1H, |
| | s), 5.69 (1H, br s), 4.25 (2H, q, J = 7.3 Hz), 3.28 (2H, q, |
| | J = 7.3 Hz), 3.00 (3H, d, J = 5.6 Hz), 1.56-1.52 (3H, m), |
| | 1.26 (3H, t, J = 7.3 Hz). |
| 91 | ¹H-NMR (CDCl₃) δ: 9.14 (1H, d, J = 0.7 Hz), 8.63 (1H, |
| | s), 8.24 (1H, br s), 7.95 (1H, s), 3.90 (3H, s), 2.65 (2H, q, |
| | J = 7.3 Hz), 1.14 (3H, t, J = 7.3 Hz). |
| 92 | ¹H-NMR (DMSO-D₆) δ: 12.05 (1H, br s), 9.64 (1H, d, |
| | J = 0.6 Hz), 8.61 (1H, s), 8.29 (1H, s), 3.79 (3H, s), 3.67 |
| | (2H, q, J = 7.4 Hz), 1.15 (3H, t, J = 7.4 Hz). |
| 93 | ¹H-NMR (CDCl₃) δ: 9.11 (1H, s), 8.67 (1H, s), 7.92 (1H, |
| | s), 4.04-4.01 (4H, m), 2.60 (2H, q, J = 7.3 Hz), 1.97-1.90 |
| | (2H, m), 1.17 (3H, t, J = 7.3 Hz), 0.98 (3H, t, J = 7.5 Hz). |
| 94 | ¹H-NMR (CDCl₃) δ: 9.14 (1H, s), 8.65 (1H, s), 7.97 (1H, |
| | s), 7.85 (1H, br s), 4.12-4.09 (2H, m), 2.67 (2H, q, J = 7.3 |
| | Hz), 2.02-1.95 (2H, m), 1.14 (3H, t, J = 7.3 Hz), 0.95 (3H, |
| | t, J = 7.5 Hz). |
| 95 | ¹H-NMR (CDCl₃) δ: 9.14 (1H, s), 8.65 (1H, s), 7.97 (1H, |
| | s), 7.95 (1H, br s), 4.10-4.07 (2H, m), 2.66 (2H, q, J = 7.3 |
| | Hz), 2.01-1.93 (2H, m), 1.14 (3H, t, J = 7.3 Hz), 0.94 (3H, |
| | t, J = 7.5 Hz). |
| 96 | ¹H-NMR (CDCl₃) δ: 9.14 (1H, s), 8.65 (1H, s), 7.97 (1H, |
| | s), 7.95 (1H, br s), 4.10-4.07 (2H, m), 2.67 (2H, q, J = 7.3 |
| | Hz), 2.00-1.93 (2H, m), 1.14 (3H, t, J = 7.3 Hz), 0.93 (3H, |
| | t, J = 7.3 Hz). |
| 97 | ¹H-NMR (DMSO-D₆) δ: 11.91 (1H, br s), 9.63 (1H, d, J = |
| | 0.7 Hz), 8.61 (1H, s), 8.29 (1H, s), 4.07 (2H, t, J = 7.0 Hz), |
| | 3.65 (2H, q, J = 7.4 Hz), 1.85-1.76 (2H, m), 1.16 (3H, t, |
| | J = 7.4 Hz), 0.85 (3H, t, J = 7.4 Hz). |
| 98 | ¹H-NMR (DMSO-D₆) δ: 12.04 (1H, br s), 9.63 (1H, d, J = |
| | 0.7 Hz), 8.62 (1H, s), 8.29 (1H, s), 4.03 (2H, t, J = 7.1 Hz), |
| | 3.65 (2H, q, J = 7.4 Hz), 1.84-1.75 (2H, m), 1.16 (3H, t, |
| | J = 7.4 Hz), 0.84 (3H, t, J = 7.4 Hz). |
| 99 | ¹H-NMR (DMSO-D₆) δ: 12.03 (1H, br s), 9.63 (1H, s), |
| | 8.62 (1H, s), 8.29 (1H, s), 4.03-4.00 (2H, m), 3.66 (2H, q, |
| | J = 7.3 Hz), 1.83-1.76 (2H, m), 1.16 (3H, t, J = 7.3 Hz), |
| | 0.84 (3H, t, J = 7.3 Hz). |
| 100 | ¹H-NMR (CDCl₃) δ: 9.12 (1H, s), 8.58 (1H, s), 7.92 (1H, |
| | s), 5.07 (2H, s), 3.77 (3H, s), 3.28 (2H, q, J = 7.3 Hz), 1.28 |
| | (3H, t, J = 7.3 Hz). |
| 101 | ¹H-NMR (CDCl₃) δ: 9.13 (1H, s), 8.64 (1H, s), 7.95 (1H, |
| | s), 7.05 (1H, s), 4.09 (2H, t, J = 7.5 Hz), 2.64 (2H, q, J = |
| | 7.2 Hz), 2.02-1.94 (2H, m), 1.15 (3H, t, J = 7.2 Hz), 0.94 |
| | (3H, t, J = 7.5 Hz). |
| 102 | ¹H-NMR (CDCl₃) δ: 9.13 (1H, s), 8.50 (1H, s), 8.08 (1H, |
| | br s), 7.96 (1H, s), 4.12-4.09 (2H, m), 3.39 (2H, q, J = 7.4 |
| | Hz), 2.29 (3H, s), 2.03-1.95 (2H, m), 1.25 (3H, t, J = 7.4 |
| | Hz), 0.94 (3H, t, J = 7.4 Hz). |
| 103 | ¹H-NMR (CDCl₃) δ: 9.13 (1H, s), 8.65 (1H, s), 7.95 (1H, |
| | s), 7.01 (1H, br s), 4.10 (2H, t, J = 7.3 Hz), 2.64 (2H, q, J = |
| | 7.0 Hz), 2.56-2.51 (2H, m), 2.01-1.94 (2H, m), 1.34-1.31 |
| | (3H, m), 1.14 (3H, t, J = 7.2 Hz), 0.93 (3H, t, J = 7.3 Hz). |
| 104 | ¹H-NMR (CDCl₃) δ: 9.13 (1H, d, J = 0.7 Hz), 8.51 (1H, s), |
| | 8.10 (1H, s), 7.96 (1H, s), 4.12-4.08 (2H, m), 3.38 (2H, q, |
| | J = 7.4 Hz), 2.54 (2H, q, J = 7.4 Hz), 2.04-1.94 (2H, m), |
| | 1.29 (3H, t, J = 7.4 Hz), 1.24 (3H, t, J = 7.4 Hz), 0.94 (3H, |
| | t, J = 7.4 Hz). |
| 105 | ¹H-NMR (CDCl₃) δ: 9.13 (1H, s), 8.65 (1H, s), 7.95 (1H, |
| | s), 7.00 (1H, br s), 4.10 (2H, t, J = 7.3 Hz), 2.64 (2H, q, J = |
| | 7.3 Hz), 2.48 (2H, t, J = 7.0 Hz), 2.01-1.94 (2H, m),1.85- |
| | 1.81 (2H, m), 1.14 (3H, t, J = 7.3 Hz), 1.08 (3H, t, J = 7.5 |
| | Hz), 0.93 (3H, t, J = 7.3 Hz). |
| 106 | ¹H-NMR (CDCl₃) δ: 9.13 (1H, d, J = 0.7 Hz), 8.51 (1H, s), |
| | 8.10 (1H, s), 7.96 (1H, s), 4.12-4.08 (2H, m), 3.38 (2H, q, |
| | J = 7.4 Hz), 2.48 (2H, t, J = 7.4 Hz), 2.03-1.94 (2H, m), |
| | 1.85-1.75 (2H, m), 1.24 (3H, t, J = 7.4 Hz), 1.05 (3H, t, J = |
| | 7.4 Hz), 0.93 (3H, t, J = 7.4 Hz). |
| 107 | ¹H-NMR (CDCl₃) δ: 9.14 (1H, s), 8.64 (1H, s), 8.04 (1H, |
| | br s), 7.95 (1H, s), 3.90 (3H, s), 2.64 (2H, q, J = 7.3 Hz), |
| | 1.96 (3H, t, J = 19.3 Hz), 1.14 (3H, t, J = 7.3 Hz). |
| 108 | ¹H-NMR (CDCl₃) δ: 9.15 (1H, s), 8.65 (1H, s), 7.96 (1H, |
| | s), 3.81 (3H, s), 2.65 (2H, q, J = 7.3 Hz), 2.38 (6H, s), 1.15 |
| | (3H, t, J = 7.3 Hz). |
| 109 | ¹H-NMR (DMSO-D₆) δ: 11.03 (1H, s), 9.63 (1H, s), 8.60 |
| | (1H, s), 8.28 (1H, s), 3.78 (3H, s), 3.64 (2H, q, J = 7.3 Hz), |
| | 1.89 (3H, t, J = 19.7 Hz), 1.16 (3H, t, J = 7.3 Hz). |
| 110 | ¹H-NMR (DMSO-D₆) δ: 9.65 (1H, s), 8.63 (1H, s), 8.31 |
| | (1H, s), 3.84 (3H, s), 3.68 (2H, q, J = 7.4 Hz), 2.31 (6H, s), |
| | 1.10 (3H, t, J = 7.3 Hz). |
| 111 | ¹H-NMR (CDCl₃) δ: 9.14 (1H, s), 8.66 (1H, s), 7.97 (1H, |
| | s), 7.87 (1H, br s), 6.15 (1H, t, J = 53.8 Hz), 4.20 (2H, q, |
| | J = 7.3 Hz), 2.66 (2H, q, J = 7.3 Hz), 1.54 (3H, t, J = 7.3 |
| | Hz), 1.15 (3H, t, J = 7.3 Hz). |
| 112 | ¹H-NMR (CDCl₃) δ: 9.14 (1H, s), 8.95 (1H, br s), 8.50 |
| | (1H, s), 7.97 (1H, s), 6.12 (1H, t, J = 53.5 Hz), 4.21 (2H, q, |
| | J = 7.4 Hz), 3.46 (2H, q, J = 7.4 Hz), 1.59-1.56 (24H, |
| | m), 1.27 (4H, t, J = 7.4 Hz). |
| 113 | ¹H-NMR (CDCl₃) δ: 9.14 (1H, s), 8.66 (1H, s), 8.02 (1H, |
| | s), 7.97 (1H, s), 6.24 (1H, tt, J = 52.7, 5.1 Hz), 4.18 (2H, q, |
| | J = 7.3 Hz), 2.65 (2H, q, J = 7.3 Hz), 1.53 (3H, t, J = 7.3 |
| | Hz), 1.14 (3H, t, J = 7.3 Hz). |
| 114 | ¹H-NMR (CDCl₃) δ: 9.14 (1H, s), 8.65 (1H, s), 7.96 (1H, |
| | s), 7.87 (1H, s), 6.15 (1H, t, J = 53.7 Hz), 4.12-4.09 (2H, |
| | m), 2.66 (2H, q, J = 7.3 Hz), 2.02-1.94 (2H, m), 1.14 (3H, |
| | t, J = 7.3 Hz), 0.94 (3H, t, J = 7.3 Hz). |
| 115 | ¹H-NMR (CDCl₃) δ: 9.19 (1H, br s), 9.14 (1H, s), 8.49 |
| | (1H, s), 7.97 (1H, s), 6.20 (1H, tt, J = 52.6, 4.7 Hz), 4.19 |
| | (2H, q, J = 7.3 Hz), 3.46 (2H, q, J = 7.3 Hz), 1.57 (3H, t, |
| | J = 7.3 Hz), 1.26 (3H, t, J = 7.3 Hz). |
| 116 | ¹H-NMR (DMSO-D₆) δ: 11.15 (1H, s), 9.63 (1H, d, J = 0.7 |
| | Hz), 8.60 (1H, s), 8.28 (1H, t, J = 0.6 Hz), 6.54 (1H, t, J = |
| | 53.0 Hz), 4.03 (2H, t, J = 7.1 Hz), 3.61 (2H, q, J = 7.3 Hz), |
| | 1.85-1.76 (2H, m), 1.16 (3H, t, J = 7.3 Hz), 0.85 (3H, t, |
| | J = 7.3 Hz). |
| 117 | ¹H-NMR (CDCl₃) δ: 9.14 (1H, d, J = 0.9 Hz), 8.62 (1H, s), |
| | 8.07 (1H, br s), 7.96 (1H, s), 3.92 (3H, s), 2.66 (2H, q, J = |
| | 7.3 Hz), 1.15 (3H, t, J = 7.3 Hz). |
| 118 | ¹H-NMR (CDCl₃) δ: 9.14 (1H, d, J = 0.6 Hz), 8.65 (1H, s), |
| | 8.27 (1H, br s), 7.95 (1H, s), 6.16 (1H, s), 3.91 (3H, s), |
| | 2.66 (2H, q, J = 7.3 Hz), 1.16 (3H, t, J = 7.3 Hz). |
| 119 | ¹H-NMR (DMSO-D₆) δ: 11.78 (1H, br s), 9.64 (1H, s), |
| | 8.61 (1H, s), 8.29 (1H, s), 3.80 (3H, s), 3.66 (2H, q, J = 7.3 |
| | Hz), 1.16 (3H, t, J = 7.3 Hz). |
| 120 | ¹H-NMR (DMSO-D₆) δ: 11.03 (1H, br s), 9.64 (1H, s), |
| | 8.60 (1H, s), 8.28 (1H, s), 6.82 (1H, s), 3.75 (3H, s), 3.62 |
| | (2H, q, J = 7.4 Hz), 1.15 (3H, t, J = 7.4 Hz). |
| 121 | ¹H-NMR (CDCl₃) δ: 9.14 (1H, s), 8.66 (1H, s), 8.04 (1H, |
| | br s), 7.96 (1H, s), 6.24 (1H, tt, J = 52.7, 5.1 Hz), 4.10- |
| | 4.07 (2H, m), 2.65 (2H, q, J = 7.3 Hz), 2.00-1.93 (2H, m), |
| | 1.14 (3H, t, J = 7.3 Hz), 0.93 (3H, t, J = 7.3 Hz). |
| 122 | ¹H-NMR (CDCl₃) δ: 9.14-9.14 (2H, m), 8.50 (1H, s), 7.97 |
| | (1H, s), 6.20 (1H, tt, J = 52.7, 4.7 Hz), 4.12-4.09 (2H, m), |
| | 3.45 (2H, q, J = 7.3 Hz), 2.03-1.96 (2H, m), 1.25 (3H, t, |
| | J = 7.3 Hz), 0.95 (3H, t, J = 7.3 Hz). |
| 123 | ¹H-NMR (CDCl₃) δ: 9.14 (1H, s), 7.93 (1H, s), 3.96 (3H, |
| | s), 3.86 (2H, q, J = 7.3 Hz), 1.34 (3H, t, J = 7.3 Hz). |
| 124 | ¹H-NMR (CDCl₃) δ: 9.10 (1H, d, J = 0.6 Hz), 7.94 (1H, s), |
| | 3.96 (3H, s), 3.86 (2H, q, J = 7.3 Hz), 1.34 (3H, t, J = 7.3 |
| | Hz). |
| 125 | ¹H-NMR (CDCl₃) δ: 9.14 (1H, d, J = 1.0 Hz), 8.63 (1H, d, |
| | J = 0.5 Hz), 8.17 (1H, br s), 7.95 (1H, s), 3.90 (3H, s), 2.65 |
| | (2H, q, J = 7.3 Hz), 1.14 (3H, t, J = 7.3 Hz). |
| 126 | ¹H-NMR (DMSO-D₆) δ: 11.92 (1H, br s), 9.64 (1H, d, J = |
| | 1.0 Hz), 8.60 (1H, s), 8.28 (1H, s), 3.79 (3H, s), 3.66 (2H, |
| | q, J = 7.3 Hz), 1.16 (3H, t, J = 7.3 Hz). |
| 127 | ¹H-NMR (CDCl₃) δ: 9.16-9.15 (2H, m, mixture), 8.59 (1H, |
| | s, minor), 8.51 (1H, s, major), 7.98 (2H, s, mixture), 4.22- |
| | 4.10 (4H, m, mixture), 3.68-3.27 (10H, m, mixture), 1.61- |
| | 1. 54 (6H, m, mixture), 1.38-1.25 (6H, m, mixture). |
| 128 | ¹H-NMR (CDCl₃) δ: 9.18 (1H, s), 9.14 (1H, s), 8.70 (1H, |
| | s), 8.69 (2H, s), 7.93 (1H, s), 5.37 (2H, s), 3.98 (2H, s), |
| | 2.63 (2H, q, J = 7.3 Hz), 1.18 (3H, t, J = 7.3 Hz). |
| 129 | ¹H-NMR (CDCl₃) δ: 9.17 (1H, s), 9.16 (1H, d, J = 0.6 Hz), |
| | 8.67-8.64 (4H, m), 7.97 (1H, s), 5.45 (2H, s), 2.68 (2H, q, |
| | J = 7.3 Hz), 1.14 (3H, t, J = 7.3 Hz). |
| 130 | ¹H-NMR (CDCl₃) δ: 9.21 (1H, s), 9.15 (1H, s), 8.76 (2H, |
| | s), 8.40 (1H, s), 7.97 (1H, s), 5.43 (2H, s), 3.59 (2H, q, J = |
| | 7.2 Hz), 1.30-1.25 (3H, m). |
| 131 | ¹H-NMR (CDCl₃) δ: 9.14 (1H, s), 8.65 (1H, s), 8.37 (1H, |
| | s), 7.95 (1H, s), 3.94 (3H, s), 2.67 (2H, q, J = 7.3 Hz), 1.16 |
| | (3H, t, J = 7.3 Hz). |
| 132 | ¹H-NMR (DMSO-D₆) δ: 11.34 (1H, s), 9.64 (1H, s), 8.61 |
| | (1H, s), 8.29 (1H, s), 3.79 (3H, s), 3.65 (2H, q, J = 7.5 Hz), |
| | 1.18 (3H, t, J = 7.5 Hz). |
| 133 | ¹H-NMR (CDCl₃) δ: 9.11 (1H, d, J = 0.7 Hz), 8.70 (1H, s), |
| | 8.36 (1H, s), 7.91 (1H, s), 3.81 (3H, s), 3.11 (3H, s), 3.08 |
| | (3H, s), 2.56 (2H, q, J = 7.3 Hz), 1.11 (3H, t, J = 7.3 Hz). |
| 134 | ¹H-NMR (CDCl₃) δ: 9.14 (1H, d, J = 0.7 Hz), 8.63 (1H, s), |
| | 7.95 (1H, s), 6.87 (1H, br s), 4.62 (2H, q, J = 8.2 Hz), 3.92 |
| | (3H, s), 2.65 (2H, q, J = 7.3 Hz), 1.15 (3H, t, J = 7.3 Hz). |
| 135 | ¹H-NMR (CDCl₃) δ: 9.13 (1H, s), 8.62 (1H, s), 7.94 (1H, |
| | s), 7.65 (1H, s), 6.30 (1H, tt, J = 55.5, 4.5 Hz), 3.86 (3H, |
| | s), 3.12 (2H, td, J = 16.3, 4.5 Hz), 2.63 (2H, q, J = 7.3 |
| | Hz), 1.13 (3H, t, J = 7.3 Hz). |
| 136 | ¹H-NMR (DMSO-D₆) δ: 10.54 (1H, s), 9.63 (1H, s), 8.58 |
| | (1H, s), 8.27 (1H, s), 6.38 (1H, tt, J = 55.7, 4.8 Hz), 3.73 |
| | (3H, s), 3.59 (2H, q, J = 7.3 Hz), 3.16 (2H, td, J = 16.7, 4.8 |
| | Hz), 1.14 (3H, t, J = 7.3 Hz). |
| 137 | ¹H-NMR (CDCl₃) δ: 9.22 (1H, d, J = 0.7 Hz), 8.78 (1H, s), |
| | 8.30 (1H, br s), 8.00 (1H, s), 5.72 (2H, q, J = 8.2 Hz), 2.63 |
| | (2H, q, J = 7.3 Hz), 1.14 (3H, t, J = 7.3 Hz). |
| 138 | ¹H-NMR (CDCl₃) δ: 9.99 (1H, br s), 9.24 (1H, s), 8.65 |
| | (1H, d, J = 0.7 Hz), 8.00 (1H, s), 5.52 (2H, q, J = 8.1 Hz), |
| | 3.02 (2H, q, J = 7.3 Hz), 1.20 (3H, t, J = 7.3 Hz). |
| 139 | ¹H-NMR (CDCl₃) δ: 9.13 (1H, d, J = 0.7 Hz), 8.65 (1H, s), |
| | 7.94 (1H, s), 7.80 (1H, br s), 3.86 (3H, s), 2.63 (2H, q, J = |
| | 7.3 Hz), 1.47-1.43 (2H, m), 1.29-1.24 (2H, m), 1.14 (3H, t, |
| | J = 7.3 Hz). |
| 140 | ¹H-NMR (CDCl₃) δ: 9.20 (1H, d, J = 0.6 Hz), 9.10 (1H, s), |
| | 8.80 (1H, s), 8.78 (2H, s), 8.34 (1H, br s), 7.98 (1H, s), |
| | 6.12 (2H, s), 2.62 (2H, q, J = 7.3 Hz), 1.15 (3H, t, J = 7.3 |
| | Hz). |
| 141 | ¹H-NMR (CDCl₃) δ: 10.04 (1H, br s), 9.23 (1H, d, J = 1.0 |
| | Hz), 9.16 (1H, s), 8.80 (2H, s), 8.66 (1H, d, J = 0.7 Hz), |
| | 8.02 (1H, s), 5.86 (2H, s), 3.00 (2H, q, J = 7.3 Hz), 1.19 |
| | (3H, t, J = 7.3 Hz). |
| 142 | ¹H-NMR (CDCl₃) δ: 9.20 (1H, s), 9.10 (1H, s), 8.80 (1H, |
| | s), 8.78 (2H, s), 8.44 (1H, s), 7.98 (1H, s), 6.12 (2H, s), |
| | 2.61 (2H, q, J = 7.3 Hz), 1.14 (3H, t, J = 7.3 Hz). |
| 143 | ¹H-NMR (CDCl₃) δ: 10.17 (1H, s), 9.23 (1H, s), 9.16 (1H, |
| | s), 8.81 (2H, s), 8.66 (1H, s), 8.03 (1H, s), 5.87 (2H, s), |
| | 2.99 (2H, q, J = 7.4 Hz), 1.18 (3H, t, J = 7.4 Hz). |
| 144 | ¹H-NMR (CDCl₃) δ: 9.14 (1H, s), 8.52 (1H, s), 7.96 (1H, |
| | s), 7.92 (1H, br s), 4.63 (2H, q, J = 8.3 Hz), 3.94 (3H, s), |
| | 3.39 (2H, q, J = 7.4 Hz), 1.26 (3H, t, J = 7.4 Hz). |
| 145 | ¹H-NMR (CDCl₃) δ: 9.13 (1H, d, J = 0.7 Hz), 8.65 (1H, s), |
| | 8.11 (1H, d, J = 4.4 Hz), 7.94 (1H, s), 3.92 (3H, s), 2.65 |
| | (2H, q, J = 7.3 Hz), 1.54 (2H, s), 1.52-1.48 (2H, m), 1.16 |
| | (3H, t, J = 7.3 Hz). |
| 146 | ¹H-NMR (DMSO-D₆) δ: 10.54 (1H, s), 9.63 (1H, s), 8.59 |
| | (1H, s), 8.27 (1H, s), 3.77 (3H, s), 3.62 (2H, q, J = 7.3 Hz), |
| | 1.55-1.47 (2H, m), 1.35-1.30 (2H, m), 1.16 (3H, t, J = 7.3 |
| | Hz). |
| 147 | ¹H-NMR (CDCl₃) δ: 9.14 (1H, s), 8.64 (1H, s), 8.15 (1H, |
| | br s), 7.95 (1H, s), 3.93 (3H, s), 2.66 (2H, q, J = 7.3 Hz), |
| | 1.15 (3H, t, J = 7.3 Hz). |
| 148 | ¹H-NMR (CDCl₃) δ: 9.15 (1H, s), 8.56 (1H, s), 7.97 (1H, |
| | s), 6.59 (1H, br s), 4.04 (3H, s), 3.18 (3H, s), 2.75 (2H, q, |
| | J = 7.3 Hz), 1.18 (3H, t, J = 7.3 Hz). |
| 149 | ¹H-NMR (CDCl₃) δ: 9.13 (1H, s), 8.69 (1H, s), 7.94 (1H, |
| | s), 4.74 (2H, q, J = 8.7 Hz), 4.16 (2H, br s), 2.63 (2H, q, |
| | J = 7.3 Hz), 1.18 (3H, t, J = 7.3 Hz). |
| 150 | ¹H-NMR (CDCl₃) δ: 9.16 (1H, s), 8.66 (1H, s), 8.16 (1H, |
| | s), 7.98 (1H, s), 4.94 (2H, q, J = 8.4 Hz), 2.71 (2H, q, J = |
| | 7.3 Hz), 1.15 (3H, t, J = 7.3 Hz). |
| 151 | ¹H-NMR (CDCl₃) δ: 9.16 (1H, s), 8.45 (1H, s), 7.98 (1H, |
| | s), 5.04 (2H, q, J = 8.3 Hz), 3.63 (2H, q, J = 7.4 Hz), 1.31 |
| | (3H, t, J = 7.4 Hz). |
| 152 | ¹H-NMR (DMSO-D₆) δ: 9.62 (1H, s), 8.76 (1H, s), 8.35 |
| | (1H, s), 8.19 (1H, s), 3.68 (3H, s), 2.96 (6H, s), 2.65 (2H, |
| | q, J = 7.3 Hz), 1.05 (3H, t, J = 7.3 Hz). |
| 153 | ¹H-NMR (DMSO-D₆) δ: 11.56 (1H, s), 9.64 (1H, d, J = 0.6 |
| | Hz), 8.61 (1H, s), 8.29 (1H, s), 3.79 (3H, s), 3.66 (2H, q, |
| | J = 7.3 Hz), 1.17 (3H, t, J = 7.3 Hz). |
| 154 | ¹H-NMR (DMSO-D₆) δ: 10.30 (1H, br s), 9.63 (1H, s), |
| | 8.57 (1H, s), 8.28 (1H, s), 3.86 (3H, s), 3.68 (2H, q, J = 7.4 |
| | Hz), 3.20 (3H, s), 1.19 (3H, t, J = 7.4 Hz). |
| 155 | ¹H-NMR (CDCl₃) δ: 9.16 (1H, s), 8.66 (1H, s), 8.26 (1H, |
| | br s), 7.98 (1H, s), 4.91 (2H, q, J = 8.4 Hz), 2.71 (2H, q, |
| | J = 7.3 Hz), 1.15 (3H, t, J = 7.3 Hz). |
| 156 | ¹H-NMR (CDCl₃) δ: 9.16 (1H, s), 8.46 (1H, s), 7.98 (1H, |
| | s), 5.03 (2H, q, J = 8.3 Hz), 3.62 (2H, q, J = 7.3 Hz), 1.30 |
| | (3H, t, J = 7.3 Hz). |
| 157 | ¹H-NMR (CDCl₃) δ: 9.16 (1H, s), 8.66 (1H, s), 8.24 (1H, |
| | br s), 7.98 (1H, s), 4.91 (2H, q, J = 8.4 Hz), 2.71 (2H, q, |
| | J = 7.3 Hz), 1.15 (3H, t, J = 7.3 Hz). |
| 158 | ¹H-NMR (CDCl₃) δ: 9.16 (1H, s), 8.45 (1H, s), 7.98 (1H, |
| | s), 5.02 (2H, q, J = 8.2 Hz), 3.63 (2H, q, J = 7.4 Hz), 1.31 |
| | (3H, t, J = 7.4 Hz). |
| 159 | ¹H-NMR (CDCl₃) δ: 9.14 (1H, s), 8.65 (1H, s), 7.97 (1H, |
| | s), 4.95 (2H, q, J = 8.5 Hz), 2.67 (2H, q, J = 7.3 Hz), 2.31 |
| | (3H, s), 1.15 (3H, t, J = 7.3 Hz). |
| 160 | ¹H-NMR (CDCl₃) δ: 9.14 (1H, s), 8.52 (1H, br s), 8.47 |
| | (1H, s), 7.97 (1H, s), 5.00 (2H, q, J = 8.5 Hz), 3.52 (2H, q, |
| | J = 7.3 Hz), 2.31 (3H, s), 1.28 (3H, t, J = 7.3 Hz). |
| 161 | ¹H-NMR (CDCl₃) δ: 9.14 (1H, s), 8.65 (1H, s), 7.97 (1H, |
| | s), 4.96 (2H, q, J = 8.5 Hz), 2.67 (2H, q, J = 7.3 Hz), 2.57- |
| | 2.51 (2H, m), 1.31 (3H, t, J = 7.6 Hz), 1.14 (3H, t, J = 7.3 |
| | Hz). |
| 162 | ¹H-NMR (CDCl₃) δ: 9.15 (1H, s), 8.55 (1H, s), 8.48 (1H, |
| | s), 7.97 (1H, s), 5.02 (2H, q, J = 8.5 Hz), 3.51 (2H, q, J = |
| | 7.4 Hz), 2.55 (2H, q, J = 7.4 Hz), 1.30 (3H, t, J = 7.4 Hz), |
| | 1.27 (3H, t, J = 7, 4 Hz). |
| 163 | ¹H-NMR (CDCl₃) δ: 9.14 (1H, s), 8.65 (1H, s), 7.97 (1H, |
| | s), 4.96 (2H, q, J = 8.5 Hz), 2.66 (2H, q, J = 7.3 Hz), 2.49 |
| | (2H, t, J = 7.3 Hz), 1.85-1.78 (2H, m), 1.14 (3H, t, J = 7.3 |
| | Hz), 1.07 (3H, t, J = 7.3 Hz). |
| 164 | ¹H-NMR (CDCl₃) δ: 9.14 (1H, s), 8.56 (1H, s), 8.47 (1H, |
| | s), 7.97 (1H, s), 5.03 (2H, q, J = 8.5 Hz), 3.51 (2H, q, J = |
| | 7.3 Hz), 2.49 (2H, t, J = 7.5 Hz), 1.83-1.76 (2H, m), 1.27 |
| | (3H, t, J = 7.3 Hz), 1.04 (3H, t, J = 7.3 Hz). |
| 165 | ¹H-NMR (CDCl₃) δ: 9.13 (1H, s), 8.63 (1H, s), 7.94 (1H, |
| | s), 7.34 (1H, br s), 6.54 (1H, d, J = 17.1 Hz), 6.47-6.30 |
| | (1H, m), 5.94 (1H, d, J = 10.5 Hz), 3.91 (3H, s), 2.63 (2H, |
| | q, J = 7.3 Hz), 1.13 (3H, t, J = 7.3 Hz). |
| 166 | ¹H-NMR (DMSO-D₆) δ: 10.39 (1H, s), 9.63 (1H, s), 8.58 |
| | (1H, s), 8.27 (1H, s), 6.51 (1H, dd, J = 17.0, 10.2 Hz), 6.33 |
| | (1H, dd, J = 17.0, 1.5 Hz), 5.89 (1H, dd, J = 10.2, 1.5 Hz), |
| | 3.73 (3H, s), 3.60 (2H, q, J = 7.3 Hz), 1.14 (3H, t, J = 7.3 |
| | Hz). |
| 167 | ¹H-NMR (CDCl₃) δ: 9.12 (1H, s), 8.52 (1H, s), 7.95 (1H, |
| | s), 7.87 (1H, s), 3.90 (3H, s), 3.37 (2H, q, J = 7.5 Hz), 3.10 |
| | (6H, s), 1.25 (3H, t, J = 7.5 Hz). |
| 168 | ¹H-NMR (CDCl₃) δ: 9.55 (1H, d, J = 0.9 Hz), 8.11 (1H, |
| | d, J = 0.9 Hz), 6.27 (1H, br s), 4.27 (2H, q, J = 7.3 Hz), |
| | 2.85 (2H, q, J = 7.3 Hz), 1.56 (3H, t, J = 7.3 Hz), 1.52 (9H, |
| | s), 1.17 (3H, t, J = 7.3 Hz). |
| 169 | ¹H-NMR (CDCl₃) δ: 9.45 (1H, br s), 8.94 (1H, s), 7.91 |
| | (1H, s), 3.97-3.92 (5H, m), 2.83 (3H, s), 1.34 (3H, t, J = |
| | 7.3 Hz). |
| 170 | ¹H-NMR (CDCl₃) δ: 9.14 (1H, s), 8.65 (1H, s), 7.95 (1H, |
| | s), 7.92 (1H, d, J = 3.7 Hz), 5.90 (1H, dd, J = 47.1, 3.6 |
| | Hz), 5.39 (1H, dd, J = 14.8, 3.6 Hz), 3.93 (3H, s), 2.64 |
| | (2H, q, J = 7.3 Hz), 1.14 (3H, t, J = 7.3 Hz). |
| 171 | ¹H-NMR (CDCl₃) δ: 9.57 (1H, d, J = 0.7 Hz), 8.13 (1H, d, |
| | J = 0.7 Hz), 7.92 (1H, br s), 4.24 (2H, q, J = 7.3 Hz), 2.89 |
| | (2H, q, J = 7.3 Hz), 1.58 (3H, t, J = 7.3 Hz), 1.16 (3H, t, |
| | J = 7.3 Hz). |
| 172 | ¹H-NMR (CDCl₃) δ: 9.55 (1H, d, J = 1.0 Hz), 9.25 (1H, br |
| | s), 8.15 (1H, d, J = 1.0 Hz), 4.25 (2H, q, J = 7.3 Hz), 3.83 |
| | (2H, q, J = 7.3 Hz), 1.63 (3H, t, J = 7.3 Hz), 1.36 (3H, t, |
| | J = 7.3 Hz). |
| 173 | ¹H-NMR (DMSO-D₆) δ: 10.68 (1H, s), 9.64-9.63 (1H, m), |
| | 8.59 (1H, s), 8.29-8.27 (1H, m), 5.79 (1H, dd, J = 47.6, 3.9 |
| | Hz), 5.57 (1H, dd, J = 15.4, 3.9 Hz), 3.78 (3H, s), 3.63 2H, |
| | q, J = 7.4 Hz), 1.16 (3H, t, J = 7.4 Hz). |
| 174 | ¹H-NMR (DMSO-D₆) δ: 9.63-9.61 (1H, m), 8.75 (1H, s), |
| | 8.19 (2H, s), 6.45 (1H, q, J = 4.7 Hz), 3.69 (3H, s), 2.68- |
| | 2.62 (5H, m), 1.05 (3H, t, J = 7.3 Hz). |
| 175 | ¹H-NMR (CDCl₃) δ: 9.57 (1H, d, J = 0.8 Hz), 8.13 (1H, d, |
| | J = 0.8 Hz), 8.03 (1H, br s), 4.22 (2H, q, J = 7.3 Hz), 2.89 |
| | (2H, q, J = 7.3 Hz), 1.57 (3H, t, J = 7.3 Hz), 1.15 (3H, t, |
| | J = 7.3 Hz). |
| 176 | ¹H-NMR (CDCl₃) δ: 9.55 (1H, d, J = 1.0 Hz), 9.40 (1H, br |
| | s), 8.15 (1H, d, J = 1.0 Hz), 4.23 (2H, q, J = 7.3 Hz), 3.83 |
| | (2H, q, J = 7.3 Hz), 1.61 (3H, t, J = 7.3 Hz), 1.35 (3H, t, |
| | J = 7.3 Hz). |
| 177 | ¹H-NMR (CDCl₃) δ: 9.57 (1H, d, J = 0.8 Hz), 8.13 (1H, d, |
| | J = 0.8 Hz), 8.01 (1H, br s), 4.22 (2H, q, J = 7.3 Hz), 2.89 |
| | (2H, q, J = 7.3 Hz), 1.56 (3H, t, J = 7.3 Hz), 1.16 (3H, t, |
| | J = 7.3 Hz). |
| 178 | ¹H-NMR (CDCl₃) δ: 9.55 (1H, d, J = 1.0 Hz), 9.39 (1H, br |
| | s), 8.15 (1H, d, J = 1.0 Hz), 4.23 (2H, q, J = 7.2 Hz), 3.84 |
| | (2H, q, J = 7.4 Hz), 1.61 (3H, t, J = 7.2 Hz), 1.36 (3H, t, |
| | J = 7.4 Hz). |
| 179 | ¹H-NMR (CDCl₃) δ: 9.57-9.56 (1H, m), 8.38 (1H, s), 8.13- |
| | 8.13 (1H, m), 3.99 (3H, s), 2.89 (2H, q, J = 7.3 Hz), 1.17 |
| | (3H, t, J = 7.3 Hz). |
| 180 | ¹H-NMR (CDCl₃) δ: 9.56-9.56 (1H, m), 8.13-8.12 (2H, |
| | m), 6.24 (1H, tt, J = 52.6, 5.0 Hz), 3.95 (3H, s), 2.87 (2H, |
| | q, J = 7.4 Hz), 1.15 (3H, t, J = 7.4 Hz). |
| 181 | ¹H-NMR (CDCl₃) δ: 9.51-9.50 (1H, m), 8.10-8.10 (1H, |
| | m), 5.18 (2H, s), 3.80 (3H, s), 3.79 (2H, q, J = 7.5 Hz), |
| | 1.39 (3H, t, J = 7.5 Hz). |
| 182 | ¹H-NMR (DMSO-D₆) δ: 11.45 (1H, s), 10.10-10.09 (1H, |
| | m), 8.70-8.69 (1H, m), 3.85 (3H, s), 3.78 (2H, q, J = 7.4 |
| | Hz), 1.25 (3H, t, J = 7.4 Hz). |
| 183 | ¹H-NMR (DMSO-D₆) δ: 11.81 (1H, s), 10.09-10.09 (1H, |
| | m), 8.70-8.69 (1H, m), 6.87 (1H, tt, J = 51.5, 5.4 Hz), 3.83 |
| | (3H, s), 3.77 (2H, q, J = 7.4 Hz), 1.22 (3H, t, J = 7.4 Hz). |
| 184 | ¹H-NMR (CDCl₃) δ: 9.57 (1H, d, J = 1.1 Hz), 8.13 (1H, d, |
| | J = 1.1 Hz), 8.06 (1H, br s), 6.24 (1H, tt, J = 52.7, 5.0 Hz), |
| | 4.22 (2H, q, J = 7.3 Hz), 2.88 (2H, q, J = 7.3 Hz), 1.56 |
| | (3H, t, J = 7.3 Hz), 1.15 (3H, t, J = 7.3 Hz). |
| 185 | ¹H-NMR (CDCl₃) δ: 9.55 (1H, d, J = 1.0 Hz), 9.31 (1H, br |
| | s), 8.15 (1H, d, J = 1.0 Hz), 6.21 (1H, tt, J = 52.7, 4.8 Hz), |
| | 4.23 (2H, q, J = 7.3 Hz), 3.81 (2H, q, J = 7.4 Hz), 1.61 |
| | (3H, t, J = 7.3 Hz), 1.34 (3H, t, J = 7.4 Hz). |
| 186 | ¹H-NMR (CDCl₃) δ: 9.57 (1H, d, J = 1.0 Hz), 8.29 (1H, br |
| | s), 8.13 (1H, d, J = 1.0 Hz), 4.25 (2H, q, J = 7.3 Hz), 2.89 |
| | (2H, q, J = 7.3 Hz), 1.59 (3H, t, J = 7.3 Hz), 1.18 (3H, t, |
| | J = 7.3 Hz). |
| 187 | ¹H-NMR (CDCl₃) δ: 9.58 (1H, br s), 9.55 (1H, d, J = 1.0 |
| | Hz), 8.15 (1H, d, J = 1.0 Hz), 4.25 (2H, q, J = 7.2 Hz), |
| | 3.82 (2H, q, J = 7.4 Hz), 1.64 (3H, t, J = 7.2 Hz), 1.37 (3H, |
| | t, J = 7.4 Hz). |
| 188 | ¹H-NMR (CDCl₃) δ: 9.56 (1H, s), 8.12 (1H, s), 4.23 (2H, |
| | q, J = 7.3 Hz), 2.85 (2H, q, J = 7.3 Hz), 2.32 (3H, s), 1.55 |
| | (3H, t, J = 7.3 Hz), 1.16 (3H, t, J = 7.3 Hz). |
| 189 | ¹H-NMR (CDCl₃) δ: 9.54 (1H, d, J = 1.0 Hz), 8.25 (1H, br |
| | s), 8.14 (1H, d, J = 1.0 Hz), 4.23 (2H, q, J = 7.3 Hz), 3, 79 |
| | (2H, q, J = 7.3 Hz), 2.31 (3H, s), 1.59 (3H, t, J = 7. 3 Hz), |
| | 1.35 (3H, t, J = 7.3 Hz). |
| 190 | ¹H-NMR (CDCl₃) δ: 9.14 (1H, s), 8.64 (1H, s), 7.95 (1H, |
| | s), 7.93 (2H, d, J = 8.6 Hz), 7.82 (1H, s), 7.55 (2H, d, J = |
| | 8.6 Hz), 3.96 (3H, s), 2.63 (2H, q, J = 7.4 Hz), 1.12 (3 H, |
| | t, J = 7.4 Hz). |
| 191 | ¹H-NMR (CDCl₃) δ: 9.15 (2H, d, J = 7.0 Hz), 8.54 (1H, s), |
| | 8.01-7.89 (3H, m), 7.54 (2H, d, J = 8.9 Hz), 3.99 (3H, s), |
| | 3.39 (2H, q, J = 7.0 Hz), 1.22 (3H, t, J = 7.0 Hz). |
| 192 | ¹H-NMR (CDCl₃) δ: 9.14 (1H, s), 8.65 (1H, s), 7.97 (1H, |
| | t, J = 1.8 Hz), 7.95 (1H, s), 7.85 (1H, d, J = 7.4 Hz), 7.81 |
| | (1H, s), 7.64 (1H, ddd, J = 8.0, 2.1, 1.2 Hz), 7.52 (1H, t, |
| | J = 7.8 Hz), 3.96 (3H, s), 2.64 (2H, q, J = 7.4 Hz), 1.13 |
| | (3H, t, J = 7.4 Hz). |
| 193 | ¹H-NMR (CDCl₃) δ: 8.46 (1H, s), 7.83 (1H, s), 4.02 (2H, |
| | br s), 3.81 (3H, s), 2.94 (3H, s), 2.62 (2H, q, J = 7.4 Hz), |
| | 1.18 (3H, t, J = 7.4 Hz). |
| 194 | ¹H-NMR (CDCl₃) δ: 8.43-8.43 (1H, m), 8.09 (1H, br s), |
| | 7.87 (1H, s), 3.91 (3H, s), 2.95 (3H, s), 2.68 (2H, q, J = 7.4 |
| | Hz), 1.14 (3H, t, J = 7.4 Hz). |
| 195 | ¹H-NMR (CDCl₃) δ: 9.14 (1H, s), 8.65 (1H, s), 8.26 (1H, |
| | s), 8.17 (1H, d, J = 7.8 Hz), 7.95-7.90 (3H, m), 7.73 (1H, |
| | t, J = 7.8 Hz), 3.97 (3H, s), 2.64 (2H, q, J = 7.4 Hz), 1.13 |
| | (3H, t, J = 7.4 Hz). |
| 196 | ¹H-NMR (CDCl₃) δ: 9.14 (1H, s), 8.68 (1H, s), 8.01 (1H, |
| | s), 7.95 (1H, s), 7.88 (1H, dd, J = 7.4, 1.5 Hz), 7.56-7.49 |
| | (2H, m), 7.46 (1H, td, J = 7.4, 1.5 Hz), 4.01 (3H, s), 2.68 |
| | (2H, q, J = 7.4 Hz), 1.18 (3H, t, J = 7.4 Hz). |
| 197 | ¹H-NMR (CDCl₃) δ: 9.15 (1H, d, J = 1.2 Hz), 9.14 (1H, s), |
| | 8.54 (1H, s), 8.00 (1H, t, J = 1.8 Hz), 7.97 (1H, s), 7.86 |
| | (1H, ddd, J = 7.8, 1.8, 1.2 Hz), 7.64 (1H, ddd, J = 7.8, 1.8, |
| | 1.2 Hz), 7.51 (1H, t, J = 7.8 Hz), 3.99 (3H, s), 3.40 (2H, q, |
| | J = 7.4 Hz), 1.23 (3H, t, J = 7.4 Hz). |
| 198 | ¹H-NMR (CDCl₃) δ: 9.14 (1H, s), 8.64 (1H, s), 7.94 (1H, |
| | s), 7.85 (1H, d, J = 7.2 Hz), 7.79 (1H, d, J = 7.2 Hz), 7.77- |
| | 7.66 (2H, m), 7.50 (1H, s), 4.02 (3H, s), 2.70 (2H, q, J = |
| | 7.3 Hz), 1.19 (3H, t, J = 7.3 Hz). |
| 199 | ¹H-NMR (CDCl₃) δ: 9.14 (1H, s), 8.65 (1H, s), 8.11 (2H, |
| | d, J = 7.8 Hz), 7.96 (1H, s), 7.85 (2H, d, J = 7.8 Hz), 7.86 |
| | (1H, s), 3.98 (3H, s), 2.64 (2H, q, J = 7.3 Hz), 1.13 (3H, t, |
| | J = 7.3 Hz). |
| 200 | ¹H-NMR (CDCl₃) δ: 9.24 (1H, s), 9.15 (1H, s), 8.53 (1H, |
| | s), 8.13 (2H, d, J = 8.3 Hz), 7.97 (1H, s), 7.84 (2H, d, J = |
| | 8.3 Hz), 4.01 (3H, s), 3.42 (2H, q, J = 7.4 Hz), 1.22 (3H, d, |
| | J = 7.4 Hz). |
| 201 | ¹H-NMR (CDCl₃) δ: 9.14 (1H, s), 8.65 (1H, s), 7.95 (1H, |
| | s), 7.85 (1H, s), 7.75 (1H, d, J = 7.6 Hz), 7.69 (1H, dt, J = |
| | 8.3, 2.4 Hz), 7.56 (1H, td, J = 8.3, 5.4 Hz), 7.36 (1H, tdd, |
| | J = 8.3, 2.4, 1.0 Hz), 3.96 (3H, s), 2.63 (2H, q, J = 7.3 Hz), |
| | 1.13 (3H, t, J = 7.3 Hz). |
| 202 | ¹H-NMR (CDCl₃) δ: 9.14 (1H, s), 8.65 (1H, s), 8.04-7.98 |
| | (2H, m), 7.95 (1H, s), 7.80 (1H, s), 7.29-7.21 (2H, m), |
| | 3.96 (3H, s), 2.63 (2H, q, J = 7.3 Hz), 1.12 (3H, t, J = 7.3 |
| | Hz). |
| 203 | ¹H-NMR (CDCl₃) δ: 9.16 (1H, s), 8.70 (1H, s), 8.45 (1H, |
| | d, J = 15.6 Hz), 8.22 (1H, td, J = 7.8, 1.8 Hz), 7.97 (1H, s), |
| | 7.69-7.62 (1H, m), 7.40 (1H, td, J = 7.8, 1.2 Hz), 7.35-7.26 |
| | (1H, m), 3.99 (3H, s), 2.67 (2H, q, J = 7.2 Hz), 1.17 (3H, t, |
| | J = 7.2 Hz). |
| 204 | ¹H-NMR (CDCl₃) δ: 9.15 (1H, s), 8.77 (1H, s), 8.56 (1H, |
| | s), 7.97 (1H, s), 7.82 (1H, dd, J = 7.8, 1.8 Hz), 7.55-7.49 |
| | (2H, m), 7.46-7.42 (1H, m), 4.03 (3H, s), 3.41 (2H, q, J = |
| | 7.4 Hz), 1.29 (3H, t, J = 7.4 Hz). |
| 205 | ¹H-NMR (DMSO-D₆) δ: 12.06 (1H, br s), 8.53 (1H, s), |
| | 8.18 (1H, s), 3.82-3.77 (5H, m), 2.92 (3H, s), 1.18 (3H, t, |
| | J = 7.4 Hz). |
| 206 | ¹H-NMR (CDCl₃) δ: 9.22 (1H, s), 9.15 (1H, s), 8.54 (1H, |
| | s), 8.30 (1H, s), 8.17 (1H, d, J = 8.0 Hz), 7.97 (1H, s), 7.93 |
| | (1H, d, J = 8.0 Hz), 7.73 (1H, t, J = 7.8 Hz), 4.00 (3H, s), |
| | 3.42 (2H, q, J = 7.4 Hz), 1.23 (3H, t, J = 7.4 Hz). |
| 207 | ¹H-NMR (CDCl₃) δ: 9.16 (1H, s), 9.15 (1H, s), 8.54 (1H, |
| | s), 7.97 (1H, s), 7.77 (1H, ddd, J = 8.0, 1.5, 0.9 Hz), 7.72 |
| | (1H, ddd, J = 8.9, 2.5, 1.8 Hz), 7.56 (1H, td, J = 8.0, 5.2 |
| | Hz), 7.37 (1H, tdd, J = 8.5, 2.5, 0.9 Hz), 3.99 (3H, s), 3.40 |
| | (2H, q, J = 7.4 Hz), 1.22 (3H, d, J = 7.4 Hz). |
| 208 | ¹H-NMR (CDCl₃) δ: 9.15 (1H, s), 9.14 (1H, s), 8.54 (1H, |
| | s), 8.05-8.00 (2H, m), 7.96 (1H, s), 7.25-7.22 (2H, m), |
| | 3.99 (3H, s), 3.38 (2H, q, J = 7.4 Hz), 1.22 (3H, t, J = 7.4 |
| | Hz). |
| 209 | ¹H-NMR (CDCl₃) δ: 9.39 (1H, d, J = 13.5 Hz), 9.14 (1H, |
| | s), 8.59 (1H, s), 8.17 (1H, td, J = 7.8, 2.1 Hz), 7.96 (1H, s), |
| | 7.67-7.61 (1H, m), 7.31-7.26 (1H, m), 7.37 (1H, t, J = 8.1 |
| | Hz), 3.97 (3H, s), 3.34 (2H, q, J = 7.4 Hz), 1.24 (3H, t, J = |
| | 7.4 Hz). |
| 210 | ¹H-NMR (CDCl₃) δ: 9.15 (1H, s), 8.53 (2H, s), 7.97 (1H, |
| | s), 7.88-7.82 (2H, m), 7.77-7.68 (2H, m), 4.02 (3H, s), |
| | 3.47 (2H, q, J = 7.5 Hz), 1.31 (3H, t, J = 7.4 Hz) |
| 211 | ¹H-NMR (CDCl₃) δ: 9.14 (1H, s), 8.65 (1H, s), 8.05 (2H, |
| | d, J = 8.6 Hz), 7.95 (1H, s), 7.83 (1H, s), 7.41 (2H, d, J = |
| | 8.6 Hz), 3.97 (3H, s), 2.63 (2H, q, J = 7.4 Hz), 1.13 (3H, t, |
| | J = 7.4 Hz). |
| 212 | ¹H-NMR (CDCl₃) δ: 9.18 (1H, s), 9.15 (1H, s), 8.54 (1H, |
| | s), 8.06 (2H, d, J = 8.9 Hz), 7.97 (1H, s), 7.40 (2H, d, J = |
| | 8.0 Hz), 3.99 (3H, s), 3.40 (2H, q, J = 7.4 Hz), 1.23 (3H, t, |
| | J = 7.4 Hz). |
| 213 | ¹H-NMR (CDCl₃) δ: 9.11 (1H, s), 8.59 (1H, s), 7.93 (1H, |
| | s), 7.85 (4H, d, J = 8.9 Hz), 7.23 (4H, d, J = 8.0 Hz), 3.91 |
| | (3H, s), 2.63 (2H, q, J = 7.5 Hz), 1.13 (3H, t, J = 7.5 Hz). |
| 214 | ¹H-NMR (CDCl₃) δ: 9.15 (1H, s), 8.69 (1H, s), 7.95 (1H, |
| | s), 3.76 (3H, s), 2.75 (2H, tt, J = 11.3, 3.7 Hz), 2.62 (2H, q, |
| | J = 7.4 Hz), 1.99-1.64 (10H, m), 1.55-1.43 (4H, m), 1.25- |
| | 1.18 (6H, m), 1.14 (3H, t, J = 7.4 Hz). |
| 215 | ¹H-NMR (CDCl₃) δ: 9.13 (1H, s), 8.63 (1H, s), 7.94 (1H, |
| | s), 7.02 (1H, s), 3.89 (3H, s), 3.33 (1H, tt, J = 8.3, 8.0 Hz), |
| | 2.62 (2H, q, J = 7.4 Hz), 2.51-2.28 (4H, m), 2.14-2.06 |
| | (1H, m), 2.04-1.94 (1H, m), 1.14 (3H, t, J = 7.4 Hz). |
| 216 | ¹H-NMR (CDCl₃) δ: 9.13 (1H, s), 8.53 (1H, s), 8.29 (1H, |
| | s), 7.95 (1H, s), 3.86 (3H, s), 3.35 (2H, q, J = 7.4 Hz), 2.42 |
| | (1H, tt, J = 11.5, 3.7 Hz), 2.12-1.65 (7H, m), 1.40-1.28 |
| | (3H, m), 1.23 (3H, t, J = 7.4 Hz). |
| 217 | ¹H-NMR (CDCl₃) δ: 9.13 (1H, s), 8.53 (1H, s), 8.29 (1H, |
| | s), 7.95 (1H, s), 3.88 (3H, s), 3.35 (2H, q, J = 7.4 Hz), 2.88 |
| | (1H, tt, J = 8.3, 8, 1 Hz), 2.11-1.65 (8H, m), 1.23 (3H, t, |
| | J = 7.4 Hz). |
| 218 | ¹H-NMR (CDCl₃) δ: 9.13 (1H, s), 8.53 (1H, s), 8.18 (1H, |
| | s), 7.95 (1H, s), 3.89 (3H, s), 3.40-3.30 (3H, m), 2.46-2.30 |
| | (4H, m), 2.15-2.13 (1H, m), 2.01-1.92 (1H, m), 1, 23 (3H, |
| | t, J = 7.5 Hz). |
| 219 | ¹H-NMR (CDCl₃) δ: 8.43 (1H, s), 8.08 (1H, s), 7.87 (1H, |
| | s), 3.91 (3H, s), 2.95 (3H, s), 2.68 (2H, q, J = 7.3 Hz), 1.14 |
| | (3H, t, J = 7.3 Hz). |
| 220 | ¹H-NMR (CDCl₃) δ: 8.43 (1H, s), 7.98 (1H, s), 7.87 (1H, |
| | s), 3.92 (3H, s), 2.95 (3H, s), 2.68 (2H, q, J = 7.3 Hz), 1.14 |
| | (3H, t, J = 7.3 Hz). |
| 221 | ¹H-NMR (CDCl₃) δ: 8.42 (1H, s), 7.86 (1H, s), 7.16 (1H, |
| | br s), 3.89 (3H, s), 2.94 (3H, s), 2.66 (2H, q, J = 7.3 Hz), |
| | 2.30 (3H, s), 1.15 (3H, t, J = 7.3 Hz). |
| 222 | ¹H-NMR (DMSO-D₆) δ: 12.04 (1H, s), 8.53 (1H, s), 8.18 |
| | (1H, s), 3.82-3.77 (5H, br m), 2.92 (3H, s), 1.17 (3H, t, J = |
| | 7.4 Hz). |
| 223 | ¹H-NMR (CDCl₃) δ: 8.31 (1H, s), 8.28 (1H, br s), 7.85 |
| | (1H, s), 3.89 (3H, s), 3.48 (2H, q, J = 7.4 Hz), 2.93 (3H, s), |
| | 2.30 (3H, s), 1.26 (3H, t, J = 7.4 Hz). |
| 224 | ¹H-NMR (CDCl₃) δ: 8.42 (1H, s), 8.12 (1H, br s), 7.71 |
| | (1H, s), 3.91 (3H, s), 2.86 (3H, s), 2.66 (2H, q, J = 7.4 Hz), |
| | 1.14 (3H, t, J = 7.4 Hz). |
| 225 | ¹H-NMR (CDCl₃) δ: 8.45 (1H, s), 8.32 (1H, s), 7.87 (1H, |
| | s), 3.94 (3H, s), 2.96 (3H, s), 2.69 (2H, q, J = 7.3 Hz), 1.16 |
| | (3H, t, J = 7.3 Hz). |
| 226 | ¹H-NMR (CDCl₃) δ: 8.44 (1H, s), 8.17 (1H, s), 7.87 (1H, |
| | s), 6.24 (1H, tt, J = 52.5, 5.1 Hz), 3.90 (3H, s), 2.95 (3H, |
| | s), 2.66 (2H, q, J = 7.3 Hz), 1.13 (3H, t, J = 7.3 Hz). |
| 227 | ¹H-NMR (CDCl₃) δ: 9.29 (1H, s), 8.29-8.29 (1H, m), 7.86 |
| | (1H, s), 3.94 (3H, s), 3.57 (2H, q, J = 7.4 Hz), 2.94 (3H, s), |
| | 1.28 (3H, t, J = 7.4 Hz). |
| 228 | ¹H-NMR (DMSO-D₆) δ: 11.70 (1H, s), 8.53 (1H, s), 8.17 |
| | (1H, s), 6.98-6.75 (1H, m), 3.80-3.74 (5H, m), 2.92 (3H, |
| | s), 1.18 (3H, t, J = 7.5 Hz). |
| 229 | ¹H-NMR (CDCl₃) δ: 9.16-9.15 (1H, m), 8.62 (1H, s), 8.13 |
| | (1H, br s), 8.01-8.00 (1H, m), 3.92 (3H, s), 2.66 (2H, q, J = |
| | 7.4 Hz), 1.14 (3H, t, J = 7.4 Hz). |
| 230 | ¹H-NMR (DMSO-D₆) δ: 11.35 (1H, s), 8.54 (1H, s), 8.17 |
| | (1H, s), 3.80-3.76 (5H, m), 2.92 (3H, s), 1.20 (3H, t, J = |
| | 7.4 Hz). |
| 231 | ¹H-NMR (CDCl₃) δ: 9.11 (1H, s), 8.57 (1H, s), 8.10 (1H, |
| | s), 7.85 (2H, s), 6.67 (1H, t, J = 55.3 Hz), 3.92 (3H, s), |
| | 2.65 (2H, q, J = 7.4 Hz), 1.14 (3H, t, J = 7.4 Hz). |
| 232 | ¹H-NMR (DMSO-D₆) δ: 11.94 (1H, br s), 9.66-9.64 (1H, |
| | m), 8.60 (1H, s), 8.34 (1H, s), 3.80 (3H, s), 3.66 (2H, q, |
| | J = 7.4 Hz), 1.16 (3H, t, J = 7.4 Hz). |
| 233 | ¹H-NMR (CDCl₃) δ: 9.33 (1H, br s), 9.05 (1H, s), 8.13 |
| | (1H, s), 3.95 (3H, s), 3.66 (2H, q, J = 7.4 Hz), 3.63 (3H, s), |
| | 1.31 (3H, t, J = 7.4 Hz). |
| 234 | ¹H-NMR (CDCl₃) δ: 9.37 (1H, s), 9.34 (1H, br s), 8.00 |
| | (1H, s), 3.94 (3H, s), 3.66 (2H, dq, J = 2.4, 7.3 Hz), 3.29 |
| | (3H, s), 1.31 (3H, t, J = 7.3 Hz). |
| 235 | ¹H-NMR (CDCl₃) δ: 9.11 (1H, s), 8.58 (1H, s), 8.19 (1H, |
| | br s), 7.85 (1H, s), 6.67 (1H, t, J = 55.3 Hz), 3.90 (3H, s), |
| | 2.65 (2H, q, J = 7.4 Hz), 1.13 (3H, t, J = 7.4 Hz). |
| 236 | ¹H-NMR (CDCl₃) δ: 9.15 (1H, s), 8.64 (1H, s), 8.15 (1H, |
| | br s), 8.00 (1H, s), 6.24 (1H, tt, J = 52.7, 5.1 Hz), 3.90 |
| | (3H, s), 2.65 (2H, q, J = 7.4 Hz), 1.14 (3H, t, J = 7.4 Hz). |
| 237 | ¹H-NMR (DMSO-D₆) δ: 11.72 (1H, br s), 9.65 (1H, s), |
| | 8.61 (1H, s), 8.34 (1H, s), 6.87 (1H, t, J = 51.6 Hz), 3.77 |
| | (3H, s), 3.66 (2H, q, J = 7.4 Hz), 1.15 (3H, t, J = 7.4 Hz). |
| 238 | ¹H-NMR (CDCl₃) δ: 9.11 (1H, s), 8.45 (1H, s), 7.86 (1H, |
| | s), 6.67 (1H, t, J = 55.2 Hz), 3.95 (3H, s), 3.47 (2H, q, J = |
| | 7.5 Hz), 1.28-1.25 (3H, m). |
| 239 | ¹H-NMR (CDCl₃) δ: 9.11 (1H, s), 8.59 (1H, s), 8.17 (1H, |
| | br s), 7.85 (1H, s), 6.67 (1H, t, J = 55.3 Hz), 6.24 (1H, tt, |
| | J = 52.7, 5.1 Hz), 3.90 (3H, s), 2.64 (2H, q, J = 7.3 Hz), |
| | 1.13 (3H, t, J = 7.4 Hz). |
| 240 | ¹H-NMR (DMSO-D₆) δ: 11.70 (1H, s), 9.58 (1H, s), 8.53 |
| | (1H, s), 7.96 (1H, s), 7.12-6.77 (2H, m), 3.76 (3H, s), 3.68- |
| | 3.63 (2H, m), 1.15 (3H, t, J = 7.4 Hz). |
| 241 | ¹H-NMR (DMSO-D₆) δ: 12.05 (1H, s), 9.58 (1H, s), 8.53 |
| | (1H, s), 7.96 (1H, s), 7.01 (1H, t, J = 54.5 Hz), 3.78 (3H, |
| | s), 3.70-3.65 (2H, m), 1.17-1.13 (3H, m). |
| 242 | ¹H-NMR (CDCl₃) δ: 9.11 (1H, s), 8.58 (1H, s), 7.85 (1H, |
| | s), 6.67 (1H, t, J = 55.3 Hz), 3.90 (3H, s), 2.64 (2H, q, J = |
| | 7.4 Hz), 1.13 (3H, t, J = 7.4 Hz). |
| 243 | ¹H-NMR (DMSO-D₆) δ: 12.02 (1H, s), 9.57 (1H, s), 8.53 |
| | (1H, s), 7.96 (1H, s), 7.01 (1H, t, J = 54.6 Hz), 3.79 (3H, |
| | s), 3.68-3.65 (2H, m), 1.15 (3H, t, J = 7.3 Hz). |
| 244 | ¹H-NMR (CDCl₃) δ: 8.65 (1H, s), 7.97 (1H, br s), 7.93 |
| | (1H, s), 3.93 (3H, s), 2.69 (2H, q, J = 7.4 Hz), 1.15 (3H, t, |
| | J = 7.4 Hz). |
| 245 | ¹H-NMR (CDCl₃) δ: 8.46 (1H, d, J = 0.6 Hz), 7.97 (1H, br |
| | s), 7.59 (1H, s), 4.33 (3H, s), 3.92 (3H, s), 2.65 (2H, q, J = |
| | 7.4 Hz), 1.13 (3H, t, J = 7.4 Hz). |
| 246 | ¹H-NMR (CDCl) δ: 9.26 (1H, br s), 8.34 (1H, s), 7.59 (1H, |
| | s), 4.32 (3H, s), 3.93 (3H, s), 3.48 (2H, q, J = 7.4 Hz), 1.25 |
| | (3H, t, J = 7.4 Hz). |
| 247 | ¹H-NMR (CDCl₃) δ: 9.15 (1H, d, J = 0.9 Hz), 8.67 (1H, s), |
| | 7.97 (1H, s), 7.91 (1H, br s), 4.21 (2H, q, J = 7.4 Hz), 2.67 |
| | (2H, q, J = 7.4 Hz), 1.56 (3H, t, J = 7.4 Hz), 1.16 (3H, t, |
| | J = 7.4 Hz). |
| 248 | ¹H-NMR (CDCl₃) δ: 9.16-9.11 (2H, m), 8.50 (1H, s), 7.97 |
| | (1H, s), 4.21 (2H, q, J = 7.4 Hz), 3.48 (2H, q, J = 7.4 Hz), |
| | 1.60 (3H, t, J = 7.4 Hz), 1.28 (3H, t, J = 7.4 Hz). |
| 249 | ¹H-NMR (CDCl₃) δ: 9.29 (1H, br s), 8.48 (1H, s), 7.94 |
| | (1H, s), 3.95 (3H, s), 3.58 (2H, q, J = 7.5 Hz), 1.29 (3H, t, |
| | J = 7.5 Hz). |
| 250 | ¹H-NMR (CDCl₃) δ: 9.11 (1H, s), 8.61 (1H, s), 8.35 (1H, |
| | s), 7.85 (1H, s), 6.67 (1H, t, J = 55.3 Hz), 3.94 (3H, s), |
| | 2.67 (2H, q, J = 7.4 Hz), 1.16 (3H, t, J = 7.4 Hz). |
| 251 | ¹H-NMR (CDCl₃) δ: 9.15 (1H, s), 8.64 (1H, s), 8.24 (1H, |
| | br s), 8.00 (1H, s), 3.91 (3H, s), 2.66 (2H, q, J = 7.4 Hz), |
| | 1.14 (3H, t, J = 7.4 Hz). |
| 252 | ¹H-NMR (CDCl₃) δ: 9.15 (1H, d, J = 0.9 Hz), 8.67 (1H, s), |
| | 8.22 (1H, s), 7.97 (1H, s), 4.21 (2H, q, J = 7.3 Hz), 2.68 |
| | (2H, q, J = 7.3 Hz), 1.57 (3H, t, J = 7.3 Hz), 1.17 (3H , t, |
| | J = 7.3 Hz). |
| 253 | ¹H-NMR (DMSO-D₆) δ: 11.35 (1H, s), 9.64 (1H, s), 8.62 |
| | (1H, s), 8.29 (1H, s), 4.14-4.07 (2H, m), 3.68-3.58 (2H, |
| | m), 1.41-1.34 (3H, m), 1.19-1.15 (3H, m). |
| 254 | ¹H-NMR (DMSO-D₆) δ: 11.34 (1H, s), 9.58 (1H, s), 8.54 |
| | (1H, s), 7.96 (1H, s), 7.01 (1H, t, J = 54.5 Hz), 3.79 (3H, |
| | s), 3.68-3.63 (2H, m), 1.17 (3H, dd, J = 12.3, 7.0 Hz). |
| 255 | ¹H-NMR (DMSO-D₆) δ: 12.04 (1H, br s), 9.65 (1H, s), |
| | 8.61 (1H, s), 8.34 (1H, s), 3.80 (3H, s), 3.67 (2H, q, J = 7.4 |
| | Hz), 1.15 (3H, t, J = 7.4 Hz). |
| 256 | ¹H-NMR (CDCl₃) δ: 9.15 (1H, s), 8.63 (1H, s), 8.26 (1H, |
| | br s), 8.00 (1H, s), 3.90 (3H, s), 2.66 (2H, q, J = 7.4 Hz), |
| | 1.14 (3H, t, J = 7.4 Hz). |
| 257 | ¹H-NMR (DMSO-D₆) δ: 12.05 (1H, br s), 9.65 (1H, d, J = |
| | 0.8 Hz), 8.62 (1H, s), 8.34 (1H, s), 3.79 (3H, s), 3.68 (2H, |
| | q, J = 7.4 Hz), 1.16 (3H, t, J = 7.4 Hz). |
| 258 | ¹H-NMR (CDCl₃) δ: 8.70 (1H, s), 7.73 (1H, s), 6.30 (1H, |
| | br s), 3.91 (3H, s), 2.66 (2H, q, J = 7.4 Hz), 2.38-2.32 (1H, |
| | m), 1.52 (9H, s), 1.49-1.45 (2H, m), 1.34-1.30 (2H, m), |
| | 1.16 (3H, t, J = 7.4 Hz). |
| 259 | ¹H-NMR (CDCl₃) δ: 8.71 (1H, s), 7.71 (1H, s), 4.01 (2H, |
| | s), 3.81 (3H, s), 2.62 (2H, q, J = 7.3 Hz), 2.38-2.32 (1H, |
| | m), 1.48-1.44 (2H, m), 1, 34-1.29 (2H, m), 1.18 (3H, t, J = |
| | 7.3 Hz). |
| 260 | ¹H-NMR (CDCl₃) δ: 8.67 (1H, s), 8.04 (1H, br s), 7.75 |
| | (1H, s), 3.92 (3H, s), 2.68 (2H, q, J = 7.4 Hz), 2.38-2.32 |
| | (1H, m), 1.50-1.46 (2H, m), 1.36-1.31 (2H, m), 1.14 (3H, |
| | t, J = 7.4 Hz). |
| 261 | ¹H-NMR (CDCl₃) δ: 8.69-8.69 (1H, m), 8.10 (1H, br s), |
| | 7.75 (1H, s), 6.24 (1H, tt, J = 52.6, 5.1 Hz), 3.90 (3H, s), |
| | 2.67 (2H, q, J = 7.3 Hz), 2.38-2.32 (1H, m), 1.50-1.46 |
| | (2H, m), 1.36-1.31 (2H, m), 1.14 (3H, t, J = 7.3 Hz). |
| 262 | ¹H-NMR (CDCl₃) δ: 8.68-8.68 (1H, m), 8.11 (1H, br s), |
| | 7.75 (1H, s), 3.90 (3H, s), 2.68 (2H, q, J = 7.3 Hz), 2.38- |
| | 2.32 (1H, m), 1.50-1.46 (2H, m), 1.36-1.31 (2H, m), 1.14 |
| | (3H, t, J = 7.3 Hz). |
| 263 | ¹H-NMR (CDCl₃) δ: 8.64-8.64 (1H, m), 7.71 (1H, s), 5.07 |
| | (2H, s), 3.77 (3H, s), 3.35 (2H, q, J = 7.4 Hz), 2.37-2.32 |
| | (1H, m), 1.47-1.44 (2H, m), 1.34-1.25 (5H, m). |
| 264 | ¹H-NMR (DMSO-D₆) δ: 11.93 (1H, s), 8.84 (1H, s), 8.07 |
| | (1H, s), 3.83-3.76 (5H, m), 2.87-2.82 (1H, m), 1.30-1.22 |
| | (4H, m), 1.19 (3H, t, J = 7.4 Hz). |
| 265 | ¹H-NMR (DMSO-D₆) δ: 11.70 (1H, s), 8.84 (1H, s), 8.07 |
| | (1H, s), 6.87 (1H, tt, J = 51.6, 5.3 Hz), 3.80-3.75 (5H, m), |
| | 2.87-2.81 (1H, m), 1.30-1.22 (4H, m), 1.18 (3H, t, J = 7.4 |
| | Hz). |
| 266 | ¹H-NMR (DMSO-D₆) δ: 12.04 (1H, s), 8.83 (1H, br s), |
| | 8.06 (1H, s), 3.82-3.76 (5H, br m), 2.85-2.81 (1H, br m), |
| | 1.30-1.22 (4H, m), 1.17 (3H, t, J = 7.3 Hz). |
| 267 | ¹H-NMR (CDCl₃) δ: 9.15 (1H, d, J = 1.2 Hz), 8.65 (1H, s), |
| | 8.36 (1H, br s), 8.00 (1H, s), 3.94 (3H, s), 2.68 (2H, q, J = |
| | 7.4 Hz), 1.17 (3H, t, J = 7.4 Hz). |
| 268 | ¹H-NMR (CDCl₃) δ: 8.42 (1H, s), 8.11 (1H, br s), 7.58 |
| | (1H, s), 4.78 (2H, q, J = 7.1 Hz), 3.91 (3H, s), 2.66 (2H, q, |
| | J = 7.4 Hz), 1.58 (3H, t, J = 7.1 Hz), 1.13 (3H, t, J = 7.4 |
| | Hz). |
| 269 | ¹H-NMR (CDCl₃) δ: 9.27 (1H, br s), 8.31 (1H, s), 7.57 |
| | (1H, s), 4.78 (2H, q, J = 7.1 Hz), 3.93 (3H, s), 3.53 (2H, q, |
| | J = 7.4 Hz), 1.56 (3H, t, J = 7.1 Hz), 1.26 (3H, t, J = 7.4 |
| | Hz). |
| 270 | ¹H-NMR (CDCl₃) δ: 8.68 (1H, s), 8.17 (1H, br s), 7.75 |
| | (1H, s), 3.90 (3H, s), 2.68 (2H, q, J = 7.4 Hz), 2.38-2.32 |
| | (1H, m), 1.50-1.47 (2H, m), 1.36-1.32 (2H, m), 1.14 (3H, |
| | t, J = 7.4 Hz). |
| 271 | ¹H-NMR (DMSO-D₆) δ: 12.06 (1H, s), 8.83 (1H, s), 8.06 |
| | (1H, s), 3.81-3.72 (5H, br m), 2.86-2.80 (1H, br m), 1.30- |
| | 1.22 (4H, m), 1.17 (3H, t, J = 7.4 Hz). |
| 272 | ¹H-NMR (CDCl₃) δ: 9.32 (1H, br s), 8.30 (1H, s), 7.57 |
| | (1H, s), 4.66 (2H, t, J = 6.7 Hz), 3.93 (3H, s), 3.55 (2H, q, |
| | J = 7.4 Hz), 1.99-1.92 (2H, m), 1.27 (3H, t, J = 7.4 Hz), |
| | 1.11 (3H, t, J = 7.4 Hz). |
| 273 | ¹H-NMR (DMSO-D₆) δ: 11.35 (1H, s), 9.65 (1H, d, J = 1.1 |
| | Hz), 8.62 (1H, s), 8.34 (1H, s), 3.80 (3H, s), 3.66 (2H, q, |
| | J = 7.4 Hz), 1.18 (3H, t, J = 7.4 Hz). |
| 274 | ¹H-NMR (CDCl₃) δ: 9.16 (1H, s), 8.65 (1H, s), 8.22 (1H, |
| | br s), 8.02 (1H, s), 4.18 (2H, q, J = 7.3 Hz), 2.66 (2H, q, |
| | J = 7.4 Hz), 1.54 (3H, t, J = 7.3 Hz), 1.14 (3H, t, J = 7.4 |
| | Hz). |
| 275 | ¹H-NMR (DMSO-D₆) δ: 11.90 (1H, br s), 9.65 (1H, d, J = |
| | 1.1 Hz), 8.62 (1H, s), 8.35 (1H, s), 4.14 (2H, q, J = 7.3 |
| | Hz), 3.65 (2H, q, J = 7.4 Hz), 1.37 (3H, t, J = 7.3 Hz), 1.16 |
| | (3H, t, J = 7.4 Hz). |
| 276 | ¹H-NMR (CDCl₃) δ: 8.49 (1H, s), 8.01 (1H, br s), 7.69 |
| | (1H, s), 5.10 (2H, q, J = 8.0 Hz), 3.92 (3H, s), 2.68 (2H, q, |
| | J = 7.3 Hz), 1.14 (3H, t, J = 7.3 Hz). |
| 277 | ¹H-NMR (CDCl₃) δ: 8.64 (1H, s), 7.99 (1H, br s), 7.29 |
| | (1H, s), 4.84 (2H, q, J = 7.5 Hz), 3.91 (3H, s), 2.64 (2H, q, |
| | J = 7.4 Hz), 1.14 (3H, t, J = 7.4 Hz). |
| 278 | ¹H-NMR (CDCl₃) δ: 9.33 (1H, br s), 8.34 (1H, d, J = 0.8 |
| | Hz), 7.69 (1H, s), 5.09 (2H, q, J = 7.9 Hz), 3.93 (3H, s), |
| | 3.61 (2H, q, J = 7.4 Hz), 1.28 (3H, t, J = 7.4 Hz). |
| 279 | ¹H-NMR (CDCl₃) δ: 9.13 (1H, d, J = 1.1 Hz), 8.65 (1H, s), |
| | 7.98-7.97 (1H, m), 6.32 (1H, br s), 3.92 (3H, s), 2.64 (2H, |
| | q, J = 7.4 Hz), 1.52 (9H, s), 1.16 (3H, t, J = 7.4 Hz). |
| 280 | ¹H-NMR (CDCl₃) δ: 9.14 (1H, s), 9.01 (1H, d, J = 2.7 Hz), |
| | 8.64 (1H, s), 8.26 (1H, dd, J = 8.0, 2.7 Hz), 7.96 (1H, s), |
| | 7.94 (1H, s), 7.55 (1H, d, J = 8.0 Hz), 3.97 (3H, s), 2.64 |
| | (2H, q, J = 7.3 Hz), 1.13 (3H, t, J = 7.3 Hz). |
| 281 | ¹H-NMR (CDCl₃) δ: 9.12 (1H, s), 8.66 (1H, s), 7.95 (1H, |
| | s), 4.03 (2H, br s), 3.81 (3H, s), 2.60 (2H, q, J = 7.4 Hz), |
| | 1.18 (3H, t, J = 7.4 Hz). |
| 282 | ¹H-NMR (CDCl₃) δ: 9.72 (1H, s), 9.14 (1H, s), 8.70 (1H, |
| | s), 8.50 (1H, d, J = 8.0 Hz), 8.20 (1H, t, J = 8.0 Hz), 7.98 |
| | (1H, dd, J = 8.0, 0.8 Hz), 7.95 (1H, s), 4.00 (3H, s), 2.66 |
| | (2H, q, J = 7.4 Hz), 1.17 (3H, t, J = 7.4 Hz). |
| 283 | ¹H-NMR (CDCl₃) δ: 10.50 (1H, s), 9.14 (1H, s), 8.61 (1H, |
| | s), 8.47 (1H, d, J = 7.6 Hz), 8.19 (1H, t, J = 7.6 Hz), 7.98 |
| | (1H, dd, J = 7.6, 0.8 Hz), 7.96 (1H, s), 4.01 (3H, s), 3.32 |
| | (2H, q, J = 7.4 Hz), 1.22 (3H, t, J = 7.4 Hz). |
| 284 | ¹H-NMR (CDCl₃) δ: 9.14 (1H, d, J = 1.1 Hz), 8.65 (1H, s), |
| | 8.10 (1H, s), 7.94 (1H, s), 3.89 (3H, s), 2.65 (2H, q, J = 7.4 |
| | Hz), 1.86-1.84 (2H, m), 1.76-1.74 (2H, m), 1.16 (3H, t, |
| | J = 7.4 Hz). |
| 285 | ¹H-NMR (CDCl₃) δ: 9.14 (1H, d, J = 0.8 Hz), 9.10 (1H, br |
| | s), 8.53 (1H, s), 7.95 (1H, s), 3.89 (3H, s), 3.40 (2H, q, J = |
| | 7.4 Hz), 1.85-1.76 (4H, m), 1.26 (3H, t, J = 7.4 Hz). |
| 286 | ¹H-NMR (CDCl₃) δ: 9.12 (1H, s), 8.67 (1H, s), 7.96 (1H, |
| | s), 4.13 (2H, q, J = 7.4 Hz), 4.02 (2H, br s), 2.61 (2H, q, |
| | J = 7.3 Hz), 1.48 (3H, t, J = 7.3 Hz), 1.18 (3H, t, J = 7.4 |
| | Hz). |
| 287 | ¹H-NMR (CDCl₃) δ: 9.13 (1H, s), 8.67 (1H, s), 7.99 (1H, |
| | s), 6.19 (1H, br s), 4.23 (2H, q, J = 7.4 Hz), 2.66 (2H, q, |
| | J = 7.3 Hz), 1.54 (3H, t, J = 7.4 Hz), 1.51 (9H, s), 1.16 |
| | (3H, t, J = 7.3 Hz). |
| 288 | ¹H-NMR (CDCl₃) δ: 9.14 (1H, d, J = 0.8 Hz), 8.66 (1H, s), |
| | 7.99 (1H, br s), 7.96 (1H, s), 4.17 (2H, q, J = 7.4 Hz), 2.65 |
| | (2H, q, J = 7.4 Hz), 1.86-1.83 (2H, m), 1.76-1.73 (2H, m), |
| | 1.53 (3H, t, J = 7.4 Hz), 1.16 (3H, t, J = 7.4 Hz). |
| 289 | ¹H-NMR (CDCl₃) δ: 9.14 (1H, d, J = 0.8 Hz), 8.89 (1H, br |
| | s), 8.52 (1H, s), 7.96 (1H, s), 4.17 (2H, q, J = 7.4 Hz), 3.41 |
| | (2H, q, J = 7.4 Hz), 1.84-1.80 (2H, m), 1.79-1.75 (2H, m), |
| | 1.56 (3H, t, J = 7.4 Hz), 1.25 (3H, t, J = 7.4 Hz). |
| 290 | ¹H-NMR (CDCl₃) δ: 9.15 (1H, d, J = 1.1 Hz), 8.65 (1H, s), |
| | 8.02 (1H, br s), 8.01 (1H, s), 4.17 (2H, q, J = 7.3 Hz), 2.66 |
| | (2H, q, J = 7.3 Hz), 1.54 (3H, t, J = 7.3 Hz), 1.14 (3H, t, |
| | J = 7.3 Hz). |
| 291 | ¹H-NMR (CDCl₃) δ: 9.15 (1H, d, J = 1.5 Hz), 8.65 (1H, s), |
| | 8.01 (1H, s), 7.98 (1H, br s), 4.17 (2H, q, J = 7.3 Hz), 2.67 |
| | (2H, q, J = 7.3 Hz), 1.53 (3H, t, J = 7.3 Hz), 1.15 (3H, t, |
| | J = 7.3 Hz). |
| 292 | ¹H-NMR (DMSO-D₆) δ: 12.03 (1H, br s), 9.65 (1H, d, J = |
| | 1.1 Hz), 8.63 (1H, s), 8.35 (1H, s), 4.11 (2H, q, J = 7.3 |
| | Hz), 3.66 (2H, q, J = 7.4 Hz), 1.36 (3H, t, J = 7.3 Hz), 1.15 |
| | (3H, t, J = 7.4 Hz). |
| 293 | ¹H-NMR (CDCl₃) δ: 9.13 (1H, s), 8.58 (1H, s), 7.97 (1H, |
| | s), 5.09 (2H, br s), 4.09 (2H, q, J = 7.4 Hz), 3.31 (2H, q, |
| | J = 7.4 Hz), 1.49 (3H, t, J = 7.4 Hz), 1.28 (3H, t, J = 7.4 |
| | Hz). |
| 294 | ¹H-NMR (DMSO-D₆) δ: 12.11 (1H, br s), 9.73 (1H, s), |
| | 8.71 (1H, s), 8.42 (1H, s), 4.17 (2H, q, J = 7.3 Hz), 3.73 |
| | (2H, q, J = 7.4 Hz), 1.44 (3H, t, J = 7.3 Hz), 1.23 (3H, t, |
| | J = 7.4 Hz). |
| 295 | ¹H-NMR (CDCl₃) δ: 9.85 (1H, s), 9.13 (1H, s), 8.68 (1H, |
| | s), 8.62 (1H, dd, J = 4.6, 1.5 Hz), 7.95 (1H, s), 7.94 (1H, |
| | dd, J = 8.2, 1.3 Hz), 7.53 (1H, dd, J = 8.2, 4.4 Hz), 4.00 |
| | (3H, s), 2.65 (2H, q, J = 7.4 Hz), 1.14 (3H, t, J = 7.3 Hz). |
| 296 | ¹H-NMR (CDCl₃) δ: 10.69 (1H, s), 9.14 (1H, s), 8.65 (1H, |
| | dd, J = 4.4, 1.3 Hz), 8.60 (1H, s), 7.96 (1H, s), 7.93 (1H, |
| | dd, J = 8.2, 1.3 Hz), 7.53 (1H, dd, J = 8.2, 4.4 Hz), 4.02 |
| | (3H, s), 3.33 (2H, q, J = 7.4 Hz), 1.23 (3H, t, J = 7.4 Hz). |
| 297 | ¹H-NMR (CDCl₃) δ: 9.13 (1H, s), 8.64 (1H, d, J = 2.7 Hz), |
| | 7.95-7.87 (1H, m), 7.71-7.65 (1H, m), 7.65-7.55 (1H, br |
| | m), 7.49-7.42 (1H, m), 7.38-7.33 (1H, m), 7.35-7.30 (1H, |
| | m), 3.99 (3H, d, J = 4.5 Hz), 2.67 (2H, dq, J = 1.7, 7.3 Hz), |
| | 2.58 (3H, d, J = 1.2 Hz), 1.17 (3H, td, J = 7.3, 1.7 Hz). |
| 298 | ¹H-NMR (CDCl₃) δ: 9.13 (1H, d, J = 1.1 Hz), 8.65 (1H, s), |
| | 7.94 (1H, s), 7.89 (1H, s), 7.80 (1H, s), 7.76 (1H, d, J = 7.3 |
| | Hz), 7.48-7.42 (2H, m), 3.96 (3H, s), 2.63 (2H, q, J = 7.4 |
| | Hz), 2.48 (3H, s), 1.13 (3H, t, J = 7.4 Hz). |
| 299 | ¹H-NMR (CDCl₃) δ: 9.13 (1H, s), 8.65 (1H, s), 7.94 (1H, |
| | s), 7.88 (2H, d, J = 8.4 Hz), 7.86 (1H, s), 7.36 (2H, d, J = |
| | 8.4 Hz), 3.96 (3H, s), 2.62 (2H, q, J = 7.3 Hz), 2.47 (3H, |
| | s), 1.12 (3H, t, J = 7.3 Hz). |
| 300 | ¹H-NMR (DMSO-D₆) δ: 9.72 (1H, s), 8.68 (1H, s), 8.36 |
| | (1H, s), 7.79-7.75 (1H, m), 7.55-7.50 (1H, m), 7.46-7.40 |
| | (2H, m), 3.93 (3H, s), 3.75 (2H, q, J = 7.4 Hz), 2.54 (3H, |
| | s), 1.27 (3H, t, J = 7.4 Hz). |
| 301 | ¹H-NMR (DMSO-D₆) δ: 10.47 (1H, s), 9.59 (1H, s), 8.56 |
| | (1H, s), 8.23 (1H, s), 7.79-7.73 (2H, m), 7.44-7.40 (2H, |
| | m), 3.74 (3H, s), 3.58 (2H, q, J = 7.4 Hz), 2.36 (3H, s), |
| | 1.09 (3H, t, J = 7.4 Hz). |
| 302 | ¹H-NMR (DMSO-D₆) δ: 9.72 (1H, s), 8.68 (1H, s), 8.36 |
| | (1H, s), 7.99 (2H, d, J = 8.0 Hz), 7.46 (2H, d, J = 8.0 Hz), |
| | 3.87 (3H, s), 3.70 (2H, q, J = 7.4 Hz), 2.48 (3H, s), 1.22 |
| | (3H, t, J = 7.4 Hz). |
| 303 | ¹H-NMR (DMSO-D₆) δ: 12.87 (1H, br s), 11.72 (1H, br s), |
| | 8.33 (1H, s), 7.31 (1H, br s), 3.77 (3H, s), 2.66 (2H, q, J = |
| | 7.4 Hz), 1.03 (3H, t, J = 7.4 Hz). |
| 304 | ¹H-NMR (DMSO-D₆) δ: 12.93 (1H, br s), 11.91 (1H, br s), |
| | 8.25 (1H, s), 7.34 (1H, s), 3.81 (3H, s), 3.57 (2H, q, J = 7.4 |
| | Hz), 1.14 (3H, t, J = 7.4 Hz). |
| 305 | ¹H-NMR (CDCl₃) δ: 9.26 (1H, br s), 8.45 (1H, s), 7.28 |
| | (1H, s), 4.83 (2H, q, J = 7.5 Hz), 3.92 (3H, s), 3.51 (2H, q, |
| | J = 7.4 Hz), 1.27 (3H, t, J = 7.4 Hz). |
| 306 | ¹H-NMR (CDCl₃) δ: 9.14 (1H, d, J = 0.8 Hz), 8.67 (1H, s), |
| | 7.96 (1H, d, J = 0.8 Hz), 7.72-7.67 (1H, m), 7.45 (2H, d, |
| | J = 8.0 Hz), 7.35 (2H, d, J = 8.0 Hz), 4.30 (2H, q, J = 7.4 |
| | Hz), 2.68 (2H, t, J = 7.4 Hz), 2.59 (3H, s), 1.60 (3H, d, J = |
| | 7.4 Hz), 1.18 (3H, t, J = 7.4 Hz). |
| 307 | ¹H-NMR (CDCl₃) δ: 9.14 (1H, d, J = 0.8 Hz), 8.67 (1H, s), |
| | 7.97 (1H, d, J = 0.8 Hz), 7.81-7.75 (3H, m), 7.48-7.44 (2H, |
| | m), 4.26 (2H, q, J = 7.4 Hz), 2.63 (2H, q, J = 7.4 Hz), 2.48 |
| | (3H, s), 1.56 (3H, t, J = 7.4 Hz), 1.13 (3H, t, J = 7.4 Hz). |
| 308 | ¹H-NMR (CDCl₃) δ: 9.14 (1H, d, J = 0.8 Hz), 8.67 (1H, s), |
| | 7.97 (1H, d, J = 0.8 Hz), 7.88 (2H, d, J = 8.0 Hz), 7.76 |
| | (1H, s), 7.36 (2H, d, J = 8.0 Hz), 4.27 (2H, q, J = 7.4 Hz), |
| | 2.62 (2H, q, J = 7.4 Hz), 2.47 (3H, s), 1.57 (3H, t, J = 7.4 |
| | Hz), 1.12 (3H, t, J = 7.4 Hz). |
| 309 | ¹H-NMR (DMSO-D₆) δ: 10.53 (1H, s), 9.64 (1H, d, J = 0.8 |
| | Hz), 8.62 (1H, d, J = 0.8 Hz), 8.30 (1H, s), 7.70-7.66 (1H, |
| | m), 7.47-7.43 (1H, m), 7.38-7.33 (2H, m), 4.18 (2H, q, J = |
| | 7.3 Hz), 3.65 (2H, q, J = 7.3 Hz), 2.45 (3H, s), 1.42 (3H, t, |
| | J = 7.3 Hz), 1.19 (3H, t, J = 7.3 Hz). |
| 310 | ¹H-NMR (DMSO-D₆) δ: 10.43 (1H, s), 9.59 (1H, d, J = 0.8 |
| | Hz), 8.57 (1H, d, J = 0.8 Hz), 8.24 (1H, s), 7.77-7.73 (2H, |
| | m), 7.43-7.40 (2H, m), 4.07 (2H, d, J = 7.4 Hz), 3.55 (2H, |
| | d, J = 7.4 Hz), 2.36 (3H, s), 1.34 (3H, t, J = 7.4 Hz), 1.09 |
| | (3H, t, J = 7.4 Hz). |
| 311 | ¹H-NMR (DMSO-D₆) δ: 10.43 (1H, s), 9.64 (1H, s), 8.62 |
| | (1H, s), 8.29 (1H, s), 7.92-7.89 (2H, m), 7.40-7.36 (2H, |
| | m), 4.12 (2H, q, J = 7.4 Hz), 3.60 (2H, q, J = 7.4 Hz), 2.40 |
| | (3H, s), 1.39 (3H, t, J = 7.4 Hz), 1.14 (3H, t, J = 7.4 Hz). |
| 312 | ¹H-NMR (CDCl₃) δ: 9.81 (1H, s), 9.14 (1H, s), 8.68 (1H, |
| | s), 8.27 (1H, dd, J = 7.8, 1.7 Hz), 7.95 (1H, d, J = 1.7 Hz), |
| | 7.60 (1H, ddd, J = 7.8, 7.8, 1.7 Hz), 7.18 (1H, ddd, J = |
| | 7.8, 7.8, 1.7 Hz), 7.12 (1H, dd, J = 7.8, 1.7 Hz), 4.13 (3H, |
| | s), 3.96 (3H, s), 2.64 (2H, q, J = 7.4 Hz), 1.14 (3H, t, J = |
| | 7.4 Hz). |
| 313 | ¹H-NMR (CDCl₃) δ: 9.14 (1H, d, J = 0.8 Hz), 8.65 (1H, s), |
| | 7.95 (1H, s), 7.89 (1H, s), 7.55-7.51 (2H, m), 7.49-7.45 |
| | (1H, m), 7.20-7.17 (1H, m), 3.97 (3H, s), 3.91 (3H, s), |
| | 2.63 (2H, q, J = 7.3 Hz), 1.13 (3H, t, J = 7.3 Hz). |
| 314 | ¹H-NMR (CDCl₃) δ: 9.13 (1H, d, J = 1.1 Hz), 8.65 (1H, s), |
| | 7.98-7.93 (3H, m), 7.81 (1H, br s), 7.06-7.02 (2H, m), 3.96 |
| | (3H, s), 3.91 (3H, s), 2.62 (2H, q, J = 7.4 Hz), 1.12 (3H, t, |
| | J = 7.4 Hz). |
| 315 | ¹H-NMR (DMSO-D₆) δ: 10.42 (1H, s), 9.64 (1H, s), 8.61 |
| | (1H, s), 8.28 (1H, s), 7.91-7.87 (1H, m), 7.63-7.60 (1H, |
| | m), 7.28-7.25 (1H, m), 7.15-7.10 (1H, m), 3.98 (3H, s), |
| | 3.80 (3H, s), 3.63 (2H, q, J = 7.4 Hz), 1.16 (3H, t, J = 7.4 |
| | Hz). |
| 316 | ¹H-NMR (DMSO-D₆) δ: 10.55 (1H, s), 9.64 (1H, d, J = |
| | 0.8 Hz), 8.61 (1H, d, J = 0.8 Hz), 8.29 (1H, s), 7.62-7.46 |
| | (3H, m), 7.25-7.22 (1H, m), 3.84 (3H, s), 3.80 (3H, s), |
| | 3.63 (2H, q, J = 7.4 Hz), 1.15 (3H, t, J = 7.4 Hz). |
| 317 | ¹H-NMR (DMSO-D₆) δ: 10.38 (1H, s), 9.64 (1H, d, J = 0.8 |
| | Hz), 8.61 (1H, s), 8.28 (1H, d, J = 0.8 Hz), 8.01-7.98 (2H, |
| | m), 7.13-7.09 (2H, m), 3.85 (3H, s), 3.79 (3H, s), 3.62 |
| | (2H, d, J = 7.3 Hz), 1.14 (3H, t, J = 7.3 Hz). |
| 318 | ¹H-NMR (CDCl₃) δ: 9.69 (1H, s), 9.14 (1H, d, J = 0.8 Hz), |
| | 8.70 (1H, s), 8.28 (1H, dd, J = 7.8, 1.7 Hz), 7.97 (1H, s), |
| | 7.62-7.57 (1H, m), 7.18 (1H, ddd, J = 7.8, 7.8, 1.7 Hz), |
| | 7.12 (1H, dd, J = 7.8, 1.7 Hz), 4.27 (2H, q, J = 7.3 Hz), |
| | 4.12 (3H, s), 2.65 (2H, q, J = 7.3 Hz), 1.55 (3H, t, J = 7.3 |
| | Hz), 1.14 (3H, t, J = 7.3 Hz). |
| 319 | ¹H-NMR (CDCl₃) δ: 9.14 (1H, d, J = 0.8 Hz), 8.67 (1H, s), |
| | 7.97 (1H, s), 7.81 (1H, s), 7.54-7.51 (2H, m), 7.49-7.45 |
| | (1H, m), 7.20-7.17 (1H, m), 4.27 (2H, q, J = 7.3 Hz), 3.9 |
| | 1 (3H, s), 2.63 (2H, q, J = 7.3 Hz), 1.57 (3H, t, J = 7.3 Hz), |
| | 1.13 (3H, t, J = 7.3 Hz). |
| 320 | ¹H-NMR (CDCl₃) δ: 9.14 (1H, d, J = 1.1 Hz), 8.67 (1H, |
| | s), 7.98-7.93 (3H, m), 7.72 (1H, br s), 7.06-7.02 (2H, m), |
| | 4.26 (2H, q, J = 7.3 Hz), 3.91 (3H, s), 2.62 (2H, q, J = 7.3 |
| | Hz), 1.56 (3H, t, J = 7.3 Hz), 1.12 (3H, t, J = 7.3 Hz). |
| 321 | ¹H-NMR (DMSO-D₆) δ: 10.34 (1H, s), 9.64 (1H, d, J = 0.8 |
| | Hz), 8.62 (1H, d, J = 0.8 Hz), 8.29 (1H, s), 7.84-7.80 (1H, |
| | m), 7.61-7.58 (1H, m), 7.27-7.23 (1H, m), 7.13-7.10 (1H, |
| | m), 4.13 (2H, q, J = 7.3 Hz), 3.96 (3H, s), 3.61 (2H, q, J = |
| | 7.4 Hz), 1.42 (3H, t, J = 7.3 Hz), 1.18-1.15 (3H, m). |
| 322 | ¹H-NMR (DMSO-D₆) δ: 10.50 (1H, s), 9.65 (1H, d, J = 0.8 |
| | Hz), 8.63 (1H, d, J = 0.8 Hz), 8.29 (1H, s), 7.60-7.46 (3H, |
| | m), 7.26-7.21 (1H, m), 4.13 (2H, q, J = 7.3 Hz), 3.84 (3H, |
| | s), 3.60 (2H, q, J = 7.3 Hz), 1.40 (3H, t, J = 7.3 Hz), 1.15 |
| | (3H, q, J = 7.3 Hz). |
| 323 | ¹H-NMR (DMSO-D₆) δ: 10.41 (1H, s), 9.71 (1H, d, J = 0.8 |
| | Hz), 8.69 (1H, d, J = 0.8 Hz), 8.36 (1H, s), 8.08-8.04 (2H, |
| | m), 7.20-7.16 (2H, m), 4.18 (2H, q, J = 7.3 Hz), 3.92 (3H, |
| | s), 3.66 (2H, q, J = 7.3 Hz), 1.46 (3H, t, J = 7.3 Hz), 1.21 |
| | (3H, t, J = 7.3 Hz). |
| 324 | ¹H-NMR (CDCl₃) δ: 9.15 (1H, d, J = 1.5 Hz), 8.66 (1H, d, |
| | J = 0.8 Hz), 8.03 (1H, br s), 8.01-8.01 (1H, m), 6.24 (1H, |
| | tt, J = 52.7, 5.1 Hz), 4.17 (2H, q, J = 7.4 Hz), 2.66 (2H, q, |
| | J = 7.4 Hz), 1.53 (3H, t, J = 7.4 Hz), 1.14 (3H, t, J = 7.4 |
| | Hz). |
| 325 | ¹H-NMR (CDCl₃) δ: 9.15 (1H, d, J = 1.5 Hz), 8.67 (1H, d, |
| | J = 0.8 Hz), 8.23 (1H, br s), 8.02-8.01 (1H, m), 4.21 (2H, |
| | q, J = 7.3 Hz), 2.69 (2H, q, J = 7.3 Hz), 1.57 (3H, t, J = 7.3 |
| | Hz), 1.17 (3H, t, J = 7.3 Hz). |
| 326 | ¹H-NMR (CDCl₃) δ: 9.15 (1H, d, J = 1.5 Hz), 8.62 (1H, s), |
| | 8.16 (1H, br s), 7.99-7.98 (1H, m), 3.92 (3H, s), 2.66 (2H, |
| | q, J = 7.3 Hz), 1.14 (3H, t, J = 7.3 Hz). |
| 327 | ¹H-NMR (CDCl₃) δ: 9.15 (1H, d, J = 1.1 Hz), 8.64 (1H, s), |
| | 8.16 (1H, br s), 7.98 (1H, s), 6.24 (1H, tt, J = 52.7, 5.0 |
| | Hz), 3.90 (3H, s), 2.66 (2H, q, J = 7.3 Hz), 1.14 (3H, t, J = |
| | 7.3 Hz). |
| 328 | ¹H-NMR (DMSO-D₆) δ: 11.68 (1H, s), 9.65 (1H, d, J = 1.1 |
| | Hz), 8.62 (1H, s), 8.34 (1H, s), 6.87 (1H, tt, J = 51.4, 5.0 |
| | Hz), 4.08 (2H, q, J = 7.3 Hz), 3.64 (2H, q, J = 7.4 Hz), |
| | 1.36 (3H, t, J = 7.3 Hz), 1.16 (3H, t, J = 7.4 Hz). |
| 329 | ¹H-NMR (DMSO-D₆) δ: 11.34 (1H, s), 9.65 (1H, d, J = |
| | 1.1 Hz), 8.63 (1H, s), 8.34 (1H, s), 4.11 (2H, q, J = 7.3 |
| | Hz), 3.64 (2H, q, J = 7.3 Hz), 1.39 (3H, t, J = 7.3 Hz), 1.18 |
| | (3H, t, J = 7.3 Hz). |
| 330 | ¹H-NMR (CDCl₃) δ: 9.11 (1H, s), 8.65 (1H, s), 7.93 (1H, |
| | s), 4.32 (2H, br s), 3.29-3.23 (1H, m), 2.60 (2H, q, J = 7.4 |
| | Hz), 1.30-1.26 (2H, m), 1.17 (3H, t, J = 7.3 Hz), 1.12-1.08 |
| | (2H, m). |
| 331 | ¹H-NMR (CDCl₃) δ: 9.80 (1H, s), 9.14 (1H, s), 8.68 (1H, |
| | s), 8.61 (1H, dd, J = 5.2, 0.6 Hz), 8.30 (1H, dd, J = 2.1, 0.6 |
| | Hz), 7.95 (1H, s), 7.59 (1H, dd, J = 5.3, 2.3 Hz), 3.97 (3H, |
| | s), 2.64 (2H, q, J = 7.3 Hz), 1.13 (3H, t, J = 7.3 Hz). |
| 332 | ¹H-NMR (CDCl₃) δ: 10.61 (1H, s), 9.14 (1H, s), 8.64 (1H, |
| | dd, J = 5.2, 0.6 Hz), 8.58 (1H, s), 8.27 (1H, dd, J = 2.3, 0.8 |
| | Hz), 7.96 (1H, s), 7.58 (1H, dd, J = 5.3, 1.9 Hz), 3.99 (3H, |
| | s), 3.32 (2H, q, J = 7.5 Hz), 1.21 (3H, t, J = 7.4 Hz). |
| 333 | ¹H-NMR (CDCl₃) δ: 9.71 (1H, s), 9.13 (1H, s), 8.68 (1H, |
| | s), 8.66 (1H, dd, J = 2.3, 0.8 Hz), 8.24 (1H, dd, J = 8.4, 0.8 |
| | Hz), 7.95 (1H, s), 7.94 (1H, dd, J = 8.2, 2.5 Hz), 3.98 (3H, |
| | s), 2.64 (2H, q, J = 7.3 Hz), 1.13 (3H, t, J = 7.4 Hz). |
| 334 | ¹H-NMR (CDCl₃) δ: 10.53 (1H, s), 9.14 (1H, s), 8.69 (1H, |
| | dd, J = 2.3, 0.6 Hz), 8.59 (1H, s), 8.21 (1H, dd, J = 8.4, 0.6 |
| | Hz), 7.96 (1H, s), 7.93 (1H, dd, J = 8.4, 2.3 Hz), 4.00 (3H, |
| | s), 3.31 (2H, q, J = 7.5 Hz), 1.20 (3H, t, J = 7.5 Hz). |
| 335 | ¹H-NMR (CDCl₃) δ: 9.57 (1H, s), 9.14 (1H, s), 8.69 (1H, |
| | s), 8.22 (1H, dd, J = 7.4, 1.0 Hz), 7.95 (1H, s), 7.94 (1H, |
| | dd, J = 7.4, 8.0 Hz), 7.62 (1H, dd, J = 8.0, 1.1 Hz), 3.9 |
| | 7 (3H, s), 2.65 (2H, q, J = 7.3 Hz), 1.15 (3H, t, J = 7.4 Hz). |
| 336 | ¹H-NMR (CDCl₃) δ: 10.34 (1H, s), 9.14 (1H, s), 8.60 (1H, |
| | s), 8.19 (1H, dd, J = 7.6, 0.7 Hz), 7.96 (1H, s), 7.92 (1H, t, |
| | J = 7.6 Hz), 7.61 (1H, dd, J = 7.6, 0.7 Hz), 3.98 (3H, s), |
| | 3.32 (2H, q, J = 7.4 Hz), 1.22 (3H, t, J = 7.4 Hz). |
| 337 | ¹H-NMR (CDCl₃) δ: 9.14 (1H, s), 8.66 (1H, s), 8.61 (1H, |
| | dd, J = 4.7, 1.9 Hz), 8.35 (1H, s), 8.30 (1H, dd, J = 7.7, 1.9 |
| | Hz), 7.95 (1H, s), 7.48 (1H, dd, J = 7.7, 4.7 Hz), 3.99 (3H, |
| | s), 2.68 (2H, q, J = 7.3 Hz), 1.17 (3H, t, J = 7.3 Hz). |
| 338 | ¹H-NMR (CDCl₃) δ: 9.15 (1H, s), 8.94 (1H, s), 8.60 (1H, |
| | dd, J = 5.0, 1.9 Hz), 8.53 (1H, s), 8.20 (1H, dd, J = 7.6, 1.9 |
| | Hz), 7.97 (1H, s), 7.46 (1H, dd, J = 7.6, 5.0 Hz), 4.02 (3H, |
| | s), 3.44 (2H, q, J = 7.2 Hz), 1.26 (3H, t, J = 7.2 Hz). |
| 339 | ¹H-NMR (CDCl₃) δ: 9.14 (1H, s), 8.66 (1H, d, J = 5.0 Hz), |
| | 8.63 (1H, s), 7.98 (1H, s), 7.96 (1H, s), 7.87 (1H, s), 7.73 |
| | (1H, d, J = 5.0 Hz), 3.96 (3H, s), 2.65 (2H, d, J = 7.5 Hz), |
| | 1.13 (3H, t, J = 7.5 Hz). |
| 340 | ¹H-NMR (CDCl₃) δ: 9.26 (1H, s), 9.15 (1H, s), 8.66 (1H, |
| | dd, J = 5.0, 0.8 Hz), 8.50 (1H, s), 7.97 (1H, s), 7.89 (1H, |
| | dd, J = 1.5, 0.8 Hz), 7.74 (1H, dd, J = 5.0, 1.5 Hz), 3.99 |
| | (3H, s), 3.46 (2H, d, J = 7.3 Hz), 1.24 (3H, t, J = 7.3 Hz). |
| 341 | ¹H-NMR (CDCl₃) δ: 9.14 (1H, d, J = 0.8 Hz), 8.61 (1H, s), |
| | 8.01 (1H, br s), 7.98 (1H, s), 3.56-3.51 (1H, m), 2.67 (2H, |
| | q, J = 7.4 Hz), 1.29-1.26 (2H, m), 1.14 (3H, t, J = 7.4 Hz), |
| | 1.11-1.06 (2H, m). |
| 342 | ¹H-NMR (DMSO-D₆) δ: 11.93 (1H, br s), 9.66 (1H, d, J = |
| | 1.5 Hz), 8.62 (1H, s), 8.35 (1H, s), 3.83 (3H, s), 3.68 (2H, |
| | q, J = 7.4 Hz), 1.17 (3H, t, J = 7.4 Hz). |
| 343 | ¹H-NMR (DMSO-D₆) δ: 11.71 (1H, br s), 9.66 (1H, d, J = |
| | 1.5 Hz), 8.62 (1H, s), 8.35 (1H, s), 6.87 (1H, tt, J = 51.5, |
| | 5.2 Hz), 3.77 (3H, s), 3.67 (2H, q, J = 7.4 Hz), 1.16 (3H, t, |
| | J = 7.4 Hz). |
| 344 | ¹H-NMR (DMSO-D₆) δ: 12.01 (1H, br s), 9.62 (1H, d, J = |
| | 0.8 Hz), 8.60 (1H, s), 8.30 (1H, s), 3.68-3.61 (3H, m), 1.16 |
| | (3H, t, J = 7.4 Hz), 1.14-1.04 (4H, m). |
| 345 | ¹H-NMR (CDCl₃) δ: 9.14 (1H, s), 8.81 (1H, s), 8.72 (1H, |
| | d, J = 5.0 Hz), 8.65 (1H, s), 8.13 (1H, s), 7.95 (1H, s), 7.75 |
| | (1H, d, J = 5.0 Hz), 4.00 (3H, s), 2.68 (2H, q, J = 7. 4 Hz), |
| | 1.17 (3H, t, J = 7.4 Hz). |
| 346 | ¹H-NMR (CDCl₃) δ: 9.15 (1H, s), 8.88 (1H, s), 8.80 (1H, |
| | s), 8.71 (1H, d, J = 4.9 Hz), 8.52 (1H, s), 7.97 (1H, s), 7.70 |
| | (1H, d, J = 4.9 Hz), 4.02 (3H, s), 3.46 (2H, q, J = 7.4 Hz), |
| | 1.25 (3H, t, J = 7.4 Hz). |
| 347 | ¹H-NMR (CDCl₃) δ: 9.13 (1H, s), 8.65 (1H, s), 7.95 (1H, |
| | s), 7.85 (1H, s), 7.76 (1H, s), 7.70 (1H, d, J = 8.0 Hz), 7.31 |
| | (1H, d, J = 8.0 Hz), 3.96 (3H, s), 2.62 (2H, q, J = 7.3 Hz), |
| | 2.38 (6H, s), 1.12 (3H, t, J = 7.3 Hz). |
| 348 | ¹H-NMR (CDCl₃) δ: 9.14 (1H, s), 8.66 (1H, s), 7.95 (1H, |
| | s), 7.83 (1H, s), 7.58 (2H, s), 7.28 (1H, s), 3.96 (3H, s), |
| | 2.63 (2H, q, J = 7.3 Hz), 2.43 (6H, s), 1.13 (3H, t, J = 7.3 |
| | Hz). |
| 349 | ¹H-NMR (DMSO-D₆) δ: 10.42 (1H, s), 9.64 (1H, s), 8.61 |
| | (1H, s), 8.28 (1H, s), 7.80 (1H, s), 7.75 (1H, d, J = 8.0 Hz), |
| | 7.34 (1H, d, J = 8.0 Hz), 3.79 (3H, s), 3.62 (2H, d, J = 7.3 |
| | Hz), 2.31 (6H, s), 1.14 (3H, t, J = 7.3 Hz). |
| 350 | ¹H-NMR (DMSO-D₆) δ: 10.47 (1H, s), 9.65 (1H, s), 8.61 |
| | (1H, s), 8.28 (1H, s), 7.62 (2H, s), 7.30 (1H, s), 3.79 (3H, |
| | s), 3.63 (2H, d, J = 7.3 Hz), 2.37 (6H, s), 1.14 (3H, t, J = |
| | 7.3 Hz). |
| 351 | ¹H-NMR (CDCl₃) δ: 9.11 (1H, s), 8.52 (1H, s), 7.93 (1H, |
| | s), 5.37 (2H, s), 3.33 (2H, q, J = 7.4 Hz), 3.23-3.17 (1H, |
| | m), 1.30-1.24 (5H, m), 1.20-1.15 (2H, m). |
| 352 | ¹H-NMR (CDCl₃) δ: 9.14 (1H, s), 9.04 (1H, s), 8.69 (1H, |
| | d, J = 5.3 Hz), 8.66 (1H, s), 8.11 (1H, s), 7.94 (1H, s), 7.50 |
| | (1H, d, J = 5.3 Hz), 4.00 (3H, s), 2.68 (2H, q, J = 7.3 Hz), |
| | 1.18 (3H, t, J = 7.3 Hz). |
| 353 | ¹H-NMR (DMSO-D₆) δ: 9.65 (1H, s), 9.13 (1H, d, J = 2.0 |
| | Hz), 8.93 (1H, d, J = 2.0 Hz), 8.80 (1H, s), 8.50 (1H, s), |
| | 8.23 (1H, s), 3.80 (3H, s), 2.70 (2H, q, J = 7.3 Hz), 1.05 |
| | (3H, t, J = 7.3 Hz). |
| 354 | ¹H-NMR (DMSO-D₆) δ: 10.95 (1H, s), 9.65 (1H, s), 9.08 |
| | (1H, d, J = 1.9 Hz), 8.93 (1H, d, J = 2.3 Hz), 8.93 (1H, s), |
| | 8.62 (1H, s), 8.45 (1H, dd, J = 2.3, 1.9 Hz), 8.29 (1H, s), |
| | 3.86 (3H, s), 3.66 (2H, q, J = 7.4 Hz), 1.16 (3H, t, J = 7.4 |
| | Hz). |
| 355 | ¹H-NMR (DMSO-D₆) δ: 10.91 (1H, s), 9.65 (1H, s), 8.99 |
| | (1H, d, J = 2.5 Hz), 8.62 (1H, s), 8.38 (1H, dd, J = 8.4, 2.5 |
| | Hz), 8.29 (1H, s), 7.79 (1H, d, J = 8.4 Hz), 3.84 (3H, s), |
| | 3.65 (2H, q, J = 7.3 Hz), 1.17 (3H, t, J = 7.3 Hz). |
| 356 | ¹H-NMR (DMSO-D₆) δ: 11.02 (1H, s), 9.64 (1H, s), 9.49 |
| | (1H, d, J = 1.3 Hz), 9.18 (1H, d, J = 5.2 Hz), 8.79 (1H, s), |
| | 8.22 (1H, s), 8.16 (1H, dd, J = 5.2, 1.3 Hz), 3.77 (3H, s), |
| | 2.69 (2H, q, J = 7.3 Hz), 1.03 (3H, t, J = 7.3 Hz). |
| 357 | ¹H-NMR (DMSO-D₆) δ: 10.96 (1H, s), 9.64 (1H, s), 9.11 |
| | (1H, d, J = 5.0 Hz), 8.79 (1H, s), 8.22 (1H, s), 8.16 (1H, d, |
| | J = 5.0 Hz), 3.77 (3H, s), 2.69 (2H, q, J = 7.4 Hz), 1.03 |
| | (3H, t, J = 7.4 Hz). |
| 358 | ¹H-NMR (CDCl₃) δ: 9.13 (1H, d, J = 1.0 Hz), 8.64 (1H, d, |
| | J = 0.5 Hz), 7.95-7.89 (4H, m), 7.41-7.36 (2H, m), 3.95 |
| | (3H, s), 2.76 (2H, d, J = 7.4 Hz), 2.62 (2H, q, J = 7.3H z), |
| | 1.30 (3H, t, J = 7.4 Hz), 1.12 (3H, t, J = 7.3 Hz). |
| 359 | ¹H-NMR (DMSO-D₆) δ: 10.53 (1H, s), 9.72 (1H, s), 8.69 |
| | (1H, s), 8.35 (1H, s), 8.01 (2H, d, J = 8.4 Hz), 7.49 (2H, d, |
| | J = 8.4 Hz), 3.87 (3H, s), 3.70 (2H, q, J = 7.4 Hz), 2.78 |
| | (2H, q, J = 7.6 Hz), 1.28 (3H, d, J = 7.6 Hz), 1.22 (3H, t, |
| | J = 7.4 Hz). |
| 360 | ¹H-NMR (DMSO-D₆) δ: 11.13 (1H, s), 9.65 (1H, s), 9.51 |
| | (1H, d, J = 1.5 Hz), 9.19 (1H, d, J = 5.0 Hz), 8.62 (1H, s), |
| | 8.29 (1H, s), 8.17 (1H, dd, J = 5.0, 1.5 Hz), 3.83 (3H, s), |
| | 3.68 (2H, q, J = 7.4 Hz), 1.18 (3H, t, J = 7.4 Hz). |
| 361 | ¹H-NMR (DMSO-D₆) δ: 11.08 (1H, s), 9.65 (1H, s), 9.13 |
| | (1H, d, J = 5.0 Hz), 8.62 (1H, s), 8.29 (1H, s), 8.17 (1H, d, |
| | J = 5.0 Hz), 3.82 (3H, s), 3.67 (2H, q, J = 7.5 Hz), 1.17 |
| | (3H, t, J = 7.5 Hz). |
| 362 | ¹H-NMR (DMSO-D₆) δ: 10.84 (1H, s), 9.65 (1H, s), 9.05 |
| | (1H, d, J = 5.0 Hz), 8.80 (1H, s), 8.23 (1H, s), 7.94 (1H, d, |
| | J = 5.0 Hz), 3.77 (3H, s), 2.82 (3H, s), 2.69 (2H, q, J = 7.3 |
| | Hz), 1.03 (3H, t, J = 7.3 Hz). |
| 363 | ¹H-NMR (DMSO-D₆) δ: 10.96 (1H, s), 9.65 (1H, s), 9.06 |
| | (1H, d, J = 5.0 Hz), 8.62 (1H, s), 8.29 (1H, s), 7.95 (1H, d, |
| | J = 5.0 Hz), 3.82 (3H, s), 3.67 (2H, q, J = 7.4 Hz), 2.82 |
| | (3H, s), 1.17 (3H, t, J = 7.4 Hz). |
| 364 | ¹H-NMR (DMSO-D₆) δ: 10.95 (1H, s), 9.65 (1H, s), 9.31 |
| | (1H, s), 8.80 (1H, s), 8.23 (1H, s), 8.07 (1H, s), 3.76 (3H, |
| | s), 2.69 (2H, q, J = 7.3 Hz), 2.64 (3H, s), 1.03 (3H, t, J = |
| | 7.3 Hz). |
| 365 | ¹H-NMR (DMSO-D₆) δ: 10.53 (1H, s), 9.65 (1H, s), 8.80 |
| | (1H, s), 8.23 (1H, s), 7.97 (1H, d, J = 1.7 Hz), 7.96 (1H, s), |
| | 7.61-7.60 (1H, m), 3.76 (3H, s), 2.69 (2H, q, J = 7.4 Hz), |
| | 2.65 (3H, s), 1.04 (3H, t, J = 7.4 Hz). |
| 366 | ¹H-NMR (DMSO-D₆) δ: 10.72 (1H, s), 9.65 (1H, s), 8.61 |
| | (1H, s), 8.28 (1H, s), 7.98-7.97 (2H, m), 7.61 (1H, dd, J = |
| | 4.6, 4.2 Hz), 3.81 (3H, s), 3.66 (2H, q, J = 7.4 Hz), 2.64 |
| | (3H, s), 1.15 (3H, t, J = 7.4 Hz). |
| 367 | ¹H-NMR (CDCl₃) δ: 9.13 (1H, s), 8.62 (1H, s), 7.94 (1H, |
| | s), 7.19 (1H, s), 3.89 (3H, s), 2.64 (2H, q, J = 7.4 Hz), 2.30 |
| | (3H, s), 1.15 (3H, t, J = 7.4 Hz). |
| 368 | ¹H-NMR (CDCl₃) δ: 9.14 (1H, s), 8.62 (1H, s), 8.11 (1H, |
| | br s), 7.98 (1H, s), 3.55-3.50 (1H, m), 2.67 (2H, q, J = 7.4 |
| | Hz), 1.27-1.24 (2H, m), 1.14 (3H, t, J = 7.3 Hz), 1.09-1.04 |
| | (2H, m). |
| 369 | ¹H-NMR (CDCl₃) δ: 9.13 (1H, s), 8.61 (1H, s), 8.03 (1H, |
| | br s), 7.98 (1H, s), 3.55-3.50 (1H, m), 2.67 (2H, q, J = 7.4 |
| | Hz), 1.29-1.25 (2H, m), 1.14 (3H, t, J = 7.4 Hz), 1.09-1.04 |
| | (2H, m). |
| 370 | ¹H-NMR (DMSO-D₆) δ: 12.08 (1H, br s), 9.62 (1H, s), |
| | 8.61 (1H, s), 8.30 (1H, s), 3.65 (2H, q, J = 7.4 Hz), 3.58- |
| | 3.52 (1H, m), 1.15 (3H, t, J = 7.4 Hz), 1.13-1.04 (4H, m). |
| 371 | ¹H-NMR (DMSO-D₆) δ: 12.07 (1H, br s), 9.62 (1H, s), |
| | 8.61 (1H, s), 8.30 (1H, s), 3.65 (2H, q, J = 7.3 Hz), 3.56- |
| | 3.51 (1H, m), 1.15 (3H, t, J = 7.3 Hz), 1.13-1.03 (4H, m). |
| 372 | ¹H-NMR (CDCl₃) δ: 9.64 (1H, s), 9.29 (1H, d, J = 5.0 Hz), |
| | 9.15 (1H, s), 8.69 (1H, s), 8.41 (1H, d, J = 5.0 Hz), 7.96 |
| | (1H, s), 3.99 (3H, s), 2.66 (2H, q, J = 7.3 Hz), 1.16 (3H, t, |
| | J = 7.3 Hz). |
| 373 | ¹H-NMR (CDCl₃) δ: 10.46 (1H, s), 9.28 (1H, d, J = 5.0 |
| | Hz), 9.15 (1H, s), 8.58 (1H, s), 8.38 (1H, d, J = 5.0 Hz), |
| | 7.97 (1H, s), 4.01 (3H, s), 3.38 (2H, q, J = 7.4 Hz), 1.23 |
| | (3H, t, J = 7.4 Hz). |
| 374 | ¹H-NMR (CDCl₃) δ: 9.94 (1H, s), 9.44 (1H, dd, J = 5.0, |
| | 1.5 Hz), 9.14 (1H, s), 8.69 (1H, s), 8.43 (1H, dd, J = 8.4, |
| | 1.5 Hz), 7.96 (1H, s), 7.81 (1H, dd, J = 8.4, 5.0 Hz), 4.00 |
| | (3H, s), 2.66 (2H, q, J = 7.3 Hz), 1.14 (3H, t, J = 7.3 Hz). |
| 375 | ¹H-NMR (CDCl₃) δ: 10.62 (1H, s), 9.44 (1H, dd, J = 5.0, |
| | 1.6 Hz), 9.15 (1H, s), 8.60 (1H, s), 8.38 (1H, dd, J = 8.4, |
| | 1.6 Hz), 7.97 (1H, s), 7.78 (1H, dd, J = 8.4, 5.0 Hz), 4.01 |
| | (3H, s), 3.34 (2H, q, J = 7.4 Hz), 1.23 (3H, t, J = 7.4 Hz). |
| 376 | ¹H-NMR (CDCl₃) δ: 9.15 (1H, s), 8.63 (1H, s), 8.19 (1H, |
| | br s), 7.98 (1H, s), 3.91 (3H, s), 2.66 (2H, q, J = 7.4 Hz), |
| | 1.14 (3H, t, J = 7.4 Hz). |
| 377 | ¹H-NMR (CDCl₃) δ: 9.14 (1H, d, J = 0.8 Hz), 8.65 (1H, s), |
| | 7.95 (1H, d, J = 0.8 Hz), 7.33 (1H, s), 3.87 (3H, s), 2.80- |
| | 2.74 (2H, m), 2.66-2.58 (3H, m), 2.64 (2H, q, J = 7.3 Hz), |
| | 2.11-2.06 (2H, m), 1.15 (3H, t, J = 7.3 Hz). |
| 378 | ¹H-NMR (CDCl₃) δ: 9.14 (1H, d, J = 0.8 Hz), 8.65 (1H, s), |
| | 7.95 (1H, s), 7.33 (1H, s), 3.87 (3H, s), 2.80-2.74 (2H, m), |
| | 2.66-2.60 (2H, m), 2.64 (2H, q, J = 7.3 Hz), 2.12-2.05 (2H, |
| | m), 1.15 (3H, t, J = 7.3 Hz). |
| 379 | ¹H-NMR (DMSO-D₆) δ: 9.63 (1H, d, J = 0.7 Hz), 9.46 |
| | (1H, s), 8.58 (1H, s), 8.26 (1H, d, J = 0.7 Hz), 3.70 (3H, s), |
| | 3.57 (2H, d, J = 7.3 Hz), 1.40 (3H, s), 1.19-1.14 (2H, m) , |
| | 1.13 (3H, t, J = 7.3 Hz), 0.76-0.72 (2H, m). |
| 380 | ¹H-NMR (DMSO-D₆) δ: 10.38 (1H, s), 9.64 (1H, d, J = 0.7 |
| | Hz), 8.60 (1H, s), 8.27 (1H, d, J = 0.7 Hz), 3.71 (3H, s), |
| | 3.64 (2H, q, J = 7.3 Hz), 2.76-2.66 (2H, m), 2.50-2.41 (2H, |
| | m), 2.01-1.93 (2H, m), 1.17 (3H, t, J = 7.3 Hz). |
| 381 | ¹H-NMR (DMSO-D₆) δ: 10.88 (1H, s), 9.63 (1H, s), 9.08 |
| | (2H, d, J = 4.8 Hz), 8.79 (1H, s), 8.21 (1H, s), 7.81 (1H, t, |
| | J = 4.8 Hz), 3.77 (3H, s), 2.69 (2H, q, J = 7.4 Hz), 1.04 |
| | (3H, t, J = 7.4 Hz). |
| 382 | ¹H-NMR (CDCl₃) δ: 9.14 (1H, d, J = 1.1 Hz), 8.63 (1H, s), |
| | 8.15 (1H, br s), 7.98 (1H, d, J = 0.8 Hz), 6.25 (1H, tt, J = |
| | 52.7, 5.0 Hz), 3.54-3.49 (1H, m), 2.66 (2H, q, J = 7. Hz), |
| | 4 1.28-1.24 (2H, m), 1.14 (3H, t, J = 7.4 Hz), 1.09-1.04 |
| | (2H, m). |
| 383 | ¹H-NMR (DMSO-D₆) δ: 11.72 (1H, s), 9.62 (1H, d, J = 1.1 |
| | Hz), 8.60 (1H, s), 8.29 (1H, s), 6.88 (1H, tt, J = 51.4, 5.4 |
| | Hz), 3.63 (2H, q, J = 7.4 Hz), 3.54-3.49 (1H, m), 1.15 (3H, |
| | t, J = 7.4 Hz), 1.12-1.04 (4H, m). |
| 384 | ¹H-NMR (CDCl₃) δ: 9.10-9.09 (1H, m), 8.55 (1H, d, J = |
| | 0.8 Hz), 8.15 (1H, s), 7.83 (1H, d, J = 0.8 Hz), 6.74 (1H, |
| | s), 3.92 (3H, s), 2.65 (2H, q, J = 7.4 Hz), 1.14 (3H, t, J = |
| | 7.4 Hz). |
| 385 | ¹H-NMR (CDCl₃) δ: 9.16 (1H, d, J = 1.1 Hz), 8.64 (1H, d, |
| | J = 0.8 Hz), 8.21 (1H, br s), 7.99-7.98 (1H, m), 3.91 (3H, |
| | s), 2.67 (2H, q, J = 7.4 Hz), 1.14 (3H, t, J = 7.4 Hz). |
| 386 | ¹H-NMR (CDCl₃) δ: 9.30 (1H, br s), 9.09 (1H, d, J = 1.5 |
| | Hz), 8.42 (1H, d, J = 0.8 Hz), 7.86 (1H, s), 6.74 (1H, s), |
| | 3.94 (3H, s), 3.48 (2H, q, J = 7.4 Hz), 1.26 (3H, t, J = 7.4 |
| | Hz). |
| 387 | ¹H-NMR (DMSO-D₆) δ: 12.04 (1H, br s), 9.66 (1H, d, J = |
| | 1.1 Hz), 8.62 (1H, s), 8.35 (1H, s), 3.80 (3H, s), 3.68 (2H, |
| | q, J = 7.2 Hz), 1.16 (3H, t, J = 7.2 Hz). |
| 388 | ¹H-NMR (CDCl₃) δ: 9.59 (1H, s), 9.51 (1H, d, J = 1.3 Hz), |
| | 9.14 (1H, s), 8.91 (1H, d, J = 2, 7 Hz), 8.70 (1H, dd, J = |
| | 2.7, 1.3 Hz), 8.68 (1H, s), 7.95 (1H, s), 3.99 (3H, s), 2.65 |
| | (2H, q, J = 7.4 Hz), 1.13 (3H, t, J = 7.4 Hz). |
| 389 | ¹H-NMR (DMSO-D₆) δ: 12.14 (1H, br s), 9.74 (1H, d, J = |
| | 1.1 Hz), 8.70 (1H, s), 8.43 (1H, s), 3.87 (3H, s), 3.76 (2H, |
| | q, J = 7.4 Hz), 1.24 (3H, t, J = 7.4 Hz). |
| 390 | ¹H-NMR (CDCl₃) δ: 9.14 (1H, d, J = 1.1 Hz), 8.61 (1H, d, |
| | J = 0.8 Hz), 8.20 (1H, br s), 7.91-7.90 (1H, m), 3.92 (3H, |
| | s), 2.65 (2H, q, J = 7.4 Hz), 1.14 (3H, t, J = 7.4 Hz). |
| 391 | ¹H-NMR (DMSO-D₆) δ: 11.92 (1H, br s), 9.63 (1H, d, J = |
| | 1.5 Hz), 8.59 (1H, d, J = 0.8 Hz), 8.19 (1H, s), 3.82 (3H, |
| | s), 3.67 (2H, q, J = 7.4 Hz), 1.16 (3H, t, J = 7.4 Hz). |
| 392 | ¹H-NMR (CDCl₃) δ: 9.12 (1H, d, J = 0.8 Hz), 8.64 (1H, d, |
| | J = 0.8 Hz), 7.86-7.85 (1H, m), 4.03 (2H, br s), 3.81 (3H, |
| | s), 2.60 (2H, q, J = 7.3 Hz), 1.18 (3H, t, J = 7.3 Hz). |
| 393 | ¹H-NMR (CDCl₃) δ: 8.97-8.97 (1H, m), 8.49 (1H, d, J = |
| | 0.8 Hz), 7.67-7.67 (1H, m), 4.00 (2H, br s), 3.79 (3H, s), |
| | 2.59 (2H, q, J = 7.4 Hz), 1.76 (3H, s), 1.71 (3H, s), 1.17 |
| | (3H, t, J = 7.4 Hz). |
| 394 | ¹H-NMR (CDCl₃) δ: 10.46 (1H, s), 9.49 (1H, d, J = 1.4 |
| | Hz), 9.14 (1H, s), 8.91 (1H, d, J = 2.5 Hz), 8.73 (1H, dd, |
| | J = 2.5, 1.4 Hz), 8.58 (1H, s), 7.97 (1H, s), 4.02 (3H, s), |
| | 3.35 (2H, q, J = 7.4 Hz), 1.22 (3H, t, J = 7.4 Hz). |
| 395 | ¹H-NMR (CDCl₃) δ: 10.69 (1H, s), 9.14 (1H, s), 9.03 (2H, |
| | d, J = 4.9 Hz), 8.57 (1H, s), 7.97 (1H, s), 7.60 (1H, t, J = |
| | 4.9 Hz), 4.03 (3H, s), 3.37 (2H, q, J = 7.4 Hz), 1.23 (3H, t, |
| | J = 7.4 Hz). |
| 396 | ¹H-NMR (CDCl₃) δ: 9.01 (1H, s), 8.47 (1H, s), 8.22 (1H, |
| | br s), 7.74 (1H, s), 3.90 (3H, s), 2.64 (2H, q, J = 7.4 Hz), |
| | 1.76 (3H, s), 1.71 (3H, s), 1.13 (3H, t, J = 7.4 Hz). |
| 397 | ¹H-NMR (DMSO-D₆) δ: 9.46-9.45 (1H, m), 8.33 (1H, d, |
| | J = 0, 8 Hz), 7.50-7.50 (1H, m), 3.49 (3H, s), 3.45 (2H, q, |
| | J = 7.4 Hz), 1.71 (3H, s), 1.66 (3H, s), 1.10 (3H, t, J = 7.4 |
| | Hz). |
| 398 | ¹H-NMR (CDCl₃) δ: 9.13 (1H, s), 8.64 (1H, s), 7.94 (1H, |
| | s), 7.72 (1H, d, J = 12.2 Hz), 6.90 (1H, br s), 5.43 (1H, br |
| | s), 4.00 (2H, br s), 3.91 (3H, s), 2.63 (2H, q, J = 7.4 Hz), |
| | 1.38 (3H, t, J = 7.4 Hz), 1.14 (3H, t, J = 7.4 Hz). |
| 399 | ¹H-NMR (CDCl₃) δ: 9.12 (1H, d, J = 1.5 Hz), 8.57 (1H, s), |
| | 7.87-7.86 (1H, m), 5.08 (2H, br s), 3.77 (3H, s), 3.28 (2H, |
| | q, J = 7.4 Hz), 1.28 (3H, t, J = 7.4 Hz). |
| 400 | ¹H-NMR (CDCl₃) δ: 9.13 (1H, s), 8.54 (1H, s), 8.12 (1H, |
| | s), 7.95 (1H, s), 7.72 (1H, d, J = 11.8 Hz), 5.43 (1H, d, J = |
| | 11.8 Hz), 4.02 (2H, q, J = 7.0 Hz), 3.91 (3H, s), 3.33 (2H, |
| | q, J = 7.4 Hz), 1.40 (3H, t, J = 7.0 Hz), 1.24 (3H, t, J = 7.4 |
| | Hz). |
| 401 | ¹H-NMR (CDCl₃) δ: 9.13 (1H, s), 8.62 (1H, s), 7.94 (1H, |
| | s), 7.27 (1H, s), 3.88 (3H, s), 2.78 (2H, t, J = 7.6 Hz), 2.68- |
| | 2.55 (4H, m), 1.13 (3H, t, J = 7.4 Hz). |
| 402 | ¹H-NMR (CDCl₃) δ: 9.14 (1H, s), 8.50 (1H, s), 8.33 (1H, |
| | s), 7.95 (1H, s), 3.88 (3H, s), 3.40 (2H, q, J = 7.4 Hz), 2.79 |
| | (2H, t, J = 7.4 Hz), 2.67-2.54 (2H, m), 1.25 (3H, t, J = 7.4 |
| | Hz). |
| 403 | ¹H-NMR (CDCl₃) δ: 9.14 (1H, s), 8.66 (1H, s), 7.96 (1H, |
| | s), 7.92 (1H, s), 4.18 (2H, q, J = 7.3 Hz), 2.65 (2H, q, J = |
| | 7.3 Hz), 1.96 (3H, t, J = 19.3 Hz), 1.54 (3H, t, J = 7.3 Hz), |
| | 1.14 (3H, t, J = 7.3 Hz). |
| 404 | ¹H-NMR (CDCl₃) δ: 9.15 (1H, d, J = 0.8 Hz), 8.66 (1H, s), |
| | 8.18 (1H, s), 7.98 (1H, s), 4.19 (2H, q, J = 7.3 Hz), 2.66 |
| | (2H, q, J = 7.3 Hz), 1.53 (3H, t, J = 7.3 Hz), 1.14 (3H, t, |
| | J = 7.3 Hz). |
| 405 | ¹H-NMR (CDCl₃) δ: 9.14 (1H, s), 8.86 (1H, s), 8.51 (1H, |
| | s), 7.97 (1H, s), 4.20 (2H, q, J = 7.3 Hz), 3.43 (2H, q, J = |
| | 7.3 Hz), 1.94 (3H, t, J = 19.3 Hz), 1.57 (3H, t, J = 7.3 Hz), |
| | 1.25 (3H, t, J = 7.3 Hz). |
| 406 | ¹H-NMR (CDCl₃) δ: 9.27 (1H, s), 9.15 (1H, d, J = 0.8 Hz), |
| | 8.48 (1H, s), 7.97 (1H, s), 4.18 (2H, q, J = 7.3 Hz), 3.49 |
| | (2H, q, J = 7.3 Hz), 1.57 (3H, t, J = 7.3 Hz), 1.27 (3H, t, |
| | J = 7.3 Hz). |
| 407 | ¹H-NMR (CDCl₃) δ: 9.14 (1H, s), 9.09 (1H, s), 8.49 (1H, |
| | s), 7.97 (1H, s), 4.20 (2H, q, J = 7.3 Hz), 3.49 (2H, q, J = |
| | 7.3 Hz), 1.59 (3H, t, J = 7.3 Hz), 1.28 (3H, t, J = 7.3 Hz). |
| 408 | ¹H-NMR (DMSO-D₆) δ: 11.35 (1H, s), 9.65 (1H, s), 8.79 |
| | (1H, s), 8.27 (1H, s), 3.75 (3H, s), 2.70 (2H, q, J = 7.4 Hz), |
| | 1.05 (3H, t, J = 7.4 Hz). |
| 409 | ¹H-NMR (DMSO-D₆) δ: 11.62 (1H, s), 9.72 (1H, s), 8.86 |
| | (1H, s), 8.35 (1H, s), 3.84 (3H, s), 2.77 (2H, q, J = 7.4 Hz), |
| | 1.12 (3H, t, J = 7.3 Hz). |
| 410 | ¹H-NMR (DMSO-D₆) δ: 11.57 (1H, s), 9.65 (1H, d, J = 1.1 |
| | Hz), 8.62 (1H, s), 8.34 (1H, s), 3.79 (3H, t, J = 12.8 Hz), |
| | 3.67 (2H, q, J = 7.3 Hz), 1.17 (3H, t, J = 7.4 Hz). |
| 411 | ¹H-NMR (CDCl₃) δ: 9.14 (1H, s), 8.65 (1H, s), 8.18 (1H, |
| | br s), 8.00 (1H, s), 6.13 (1H, s), 3.91 (3H, s), 2.67 (2H, q, |
| | J = 7.4 Hz), 1.16 (3H, t, J = 7.4 Hz). |
| 412 | ¹H-NMR (CDCl₃) δ: 9.15 (1H, s), 8.63 (1H, s), 8.05 (1H, |
| | br s), 8.00 (1H, s), 3.91 (3H, s), 2.67 (2H, q, J = 7.4 Hz), |
| | 1.15 (3H, t, J = 7.4 Hz). |
| 413 | ¹H-NMR (DMSO-D₆) δ: 11.78 (1H, s), 9.65 (1H, d, J = 1.1 |
| | Hz), 8.61 (1H, s), 8.34 (1H, s), 3.78 (3H, t, J = 9.9 Hz), |
| | 3.69-3.65 (2H, m), 1.17 (3H, t, J = 7.3 Hz). |
| 414 | ¹H-NMR (CDCl₃) δ: 9.15 (1H, s), 8.64 (1H, s), 8.00 (1H, |
| | s), 7.98 (1H, s), 6.15 (1H, t, J = 53.7 Hz), 3.92 (3H, s), |
| | 2.66 (2H, q, J = 7.3 Hz), 1.15 (3H, t, J = 7.3 Hz). |
| 415 | ¹H-NMR (CDCl₃) δ: 9.15 (1H, s), 8.64 (1H, s), 8.16 (1H, |
| | s), 8.00 (1H, s), 3.91 (3H, s), 2.66 (2H, q, J = 7.4 Hz), 1.14 |
| | (3H, t, J = 7.4 Hz). |
| 416 | ¹H-NMR (DMSO-D₆) δ: 11.18 (1H, s), 9.65 (1H, s), 8.60 |
| | (1H, s), 8.33 (1H, s), 6.54 (1H, t, J = 52.9 Hz), 3.78 (3H, |
| | s), 3.64 (2H, q, J = 7.3 Hz), 1.16 (3H, t, J = 7.4 Hz). |
| 417 | ¹H-NMR (DMSO-D₆) δ: 11.94 (1H, s), 9.65 (1H, s), 8.61 |
| | (1H, s), 8.34 (1H, s), 3.79 (3H, s), 3.69-3.65 (2H, m), 1.17- |
| | 1.14 (3H, m). |
| 418 | ¹H-NMR (CDCl₃) δ: 9.14 (1H, s), 8.62 (1H, s), 7.95 (1H, |
| | s), 7.38 (1H, s), 4.04-3.90 (1H, m), 3.86 (3H, s), 2.96 (2H, |
| | d, J = 6.1 Hz), 2.63 (2H, q, J = 7.4 Hz), 1.13 (3H, t, J = |
| | 7.4 Hz). |
| 419 | H-NMR (CDCl₃) δ: 9.14 (1H, d, J = 1.1 Hz), 8.64 (1H, s), |
| | 8.00-7.99 (1H, m), 7.96 (1H, br s), 3.91 (3H, s), 2.65 (2H, |
| | q, J = 7.3 Hz), 1.96 (3H, t, J = 19.5 Hz), 1.14 (3H, t, J = |
| | 7.3 Hz). |
| 420 | ¹H-NMR (DMSO-D₆) δ: 11.04 (1H, s), 9.65 (1H, s), 8.60 |
| | (1H, s), 8.34 (1H, s), 6.82 (1H, s), 3.75 (3H, s), 3.63 (2H, |
| | q, J = 7.4 Hz), 1.15 (3H, t, J = 7.4 Hz). |
| 421 | ¹H-NMR (DMSO-D₆) δ: 12.05 (1H, br s), 9.65 (1H, s), |
| | 8.62 (1H, s), 8.34 (1H, s), 3.79 (3H, s), 3.68 (2H, q, J = 7.4 |
| | Hz), 1.16 (3H, t, J = 7.4 Hz). |
| 422 | ¹H-NMR (DMSO-D₆) δ: 11.11 (1H, br s), 9.73 (1H, d, J = |
| | 1.5 Hz), 8.68 (1H, s), 8.41 (1H, s), 3.86 (3H, s), 3.73 (2H, |
| | q, J = 7.4 Hz), 1.97 (3H, t, J = 19.7 Hz), 1.24 (3H, t, J = |
| | 7.4 Hz). |
| 423 | ¹H-NMR (CDCl₃) δ: 9.14 (1H, s), 8.67 (1H, s), 8.07 (1H, |
| | s), 7.97 (1H, s), 6.12 (1H, s), 4.19 (2H, q, J = 7.5 Hz), |
| | 2.66 (2H, q, J = 7.3 Hz), 1.54 (3H, t, J = 7.3 Hz), 1.16 (3H, |
| | t, J = 7.3 Hz). |
| 424 | ¹H-NMR (DMSO-D₆) δ: 11.02 (1H, s), 9.64 (1H, s), 8.62 |
| | (1H, s), 8.30 (1H, s), 6.82 (1H, s), 4.07 (2H, q, J = 7.4 Hz), |
| | 3.61 (2H, q, J = 7.3 Hz), 1.38 (3H, t, J = 7.3 Hz), 1.16 (3H, |
| | t, J = 7.4 Hz). |
| 425 | ¹H-NMR (CDCl₃) δ: 9.14 (1H, s), 8.65 (1H, s), 8.02 (1H, |
| | br s), 7.97 (1H, s), 4.17 (2H, q, J = 7.4 Hz), 2.66 (2H, q, |
| | J = 7.4 Hz), 1.53 (3H, t, J = 7.3 Hz), 1.14 (3H, t, J = 7.3 |
| | Hz). |
| 426 | ¹H-NMR (DMSO-D₆) δ: 12.04 (1H, br s), 9.65 (1H, s), |
| | 8.63 (1H, s), 8.31 (1H, s), 4.10 (2H, q, J = 7.3 Hz), 3.66 |
| | (2H, q, J = 7.3 Hz), 1.37 (3H, t, J = 7.3 Hz), 1.16 (3H, t, |
| | J = 7.3 Hz). |
| 427 | ¹H-NMR (CDCl₃) δ: 9.13 (1H, s), 8.63 (1H, s), 7.94 (1H, |
| | s), 7.13 (1H, br s), 3.89 (3H, s), 2.64 (2H, q, J = 7.4 Hz), |
| | 2.30-2.23 (1H, m), 1.83-1.74 (2H, m), 1.70-1.62 (2H, m), |
| | 1.15 (3H, t, J = 7.3 Hz), 1.06 (6H, t, J = 7.4 Hz). |
| 428 | ¹H-NMR (CDCl₃) δ: 9.13 (1H, s), 8.63 (1H, s), 7.94 (1H, |
| | s), 7.70 (1H, br s), 3.90 (3H, s), 2.63 (2H, q, J = 7.3 Hz), |
| | 2.43 (2H, d, J = 7.3 Hz), 1.20-1.14 (1H, m), 1.15 (3H, t, |
| | J = 7.4 Hz), 0.81-0.77 (2H, m), 0.41-0.37 (2H, m). |
| 429 | ¹H-NMR (CDCl₃) δ: 9.12 (1H, s), 8.63 (1H, s), 7.93 (1H, |
| | s), 7.14 (1H, br s), 3.88 (3H, s), 2.63 (2H, q, J = 7.3 Hz), |
| | 1.32 (6H, br s), 1.28 (6H, s), 1.18-1.12 (1H, m), 1.15 (3H, |
| | t, J = 7.3 Hz). |
| 430 | ¹H-NMR (DMSO-D₆) δ: 10.20 (1H, s), 9.64 (1H, s), 8.58 |
| | (1H, s), 8.28 (1H, s), 3.72 (3H, s), 3.61 (2H, q, J = 7.4 Hz), |
| | 2.41-2.34 (1H, m), 1.69-1.61 (2H, m), 1.54-1.47 (2H, m), |
| | 1.15 (3H, t, J = 7.4 Hz), 0.94 (6H, t, J = 7.3 Hz). |
| 431 | ¹H-NMR (DMSO-D₆) δ: 10.06 (1H, s), 9.64 (1H, s), 8.59 |
| | (1H, s), 8.28 (1H, s), 3.73 (3H, s), 3.59 (2H, q, J = 7.4 Hz), |
| | 2.30 (2H, d, J = 7.3 Hz), 1.20-1.12 (1H, m), 1.15 (3H, t, |
| | J = 7.4 Hz), 0.54-0.49 (2H, m), 0.28-0.23 (2H, m). |
| 432 | ¹H-NMR (DMSO-D₆) δ: 10.07 (1H, s), 9.64 (1H, s), 8.58 |
| | (1H, s), 8.28 (1H, s), 3.71 (3H, s), 3.59 (2H, q, J = 7.3 Hz), |
| | 1.45 (1H, s), 1.23 (6H, s), 1.22 (6H, s), 1.15 (3H, t, J = 7.3 |
| | Hz). |
| 433 | ¹H-NMR (DMSO-D₆) δ: 10.19 (1H, br s), 9.64 (1H, s), |
| | 8.57 (1H, s), 8.32 (1H, s), 3.72 (3H, s), 3.59 (2H, q, J = 7.4 |
| | Hz), 2.11 (3H, s), 1.15 (3H, t, J = 7.4 Hz). |
| 434 | ¹H-NMR (CDCl₃) δ: 9.13 (1H, s), 8.64 (1H, s), 7.94 (1H, |
| | s), 7.54 (1H, s), 3.85 (3H, s), 2.62 (2H, q, J = 7.4 Hz), 1.59 |
| | (6H, s), 1.13 (3H, t, J = 7.4 Hz). |
| 435 | ¹H-NMR (CDCl₃) δ: 9.14 (1H, s), 8.49 (1H, s), 8.47 (1H, |
| | s), 7.96 (1H, s), 3.98-3.89 (1H, m), 3.88 (3H, s), 3.41 (2H, |
| | q, J = 7.4 Hz), 2.97 (2H, d, J = 5.7 Hz), 1.24 (3H, t, J = |
| | 7.4 Hz). |
| 436 | ¹H-NMR (CDCl₃) δ: 9.13 (1H, s), 8.66 (1H, s), 8.52 (1H, |
| | s), 7.95 (1H, s), 3.84 (3H, s), 3.39 (2H, q, J = 7.4 Hz), 1.58 |
| | (6H, s), 1.23 (3H, t, J = 7.4 Hz). |

Table 9 shows the synthesis intermediates of the compounds of the present invention synthesized according to the Examples described above, but the synthesis intermediates are not limited thereto.

In Table 9, the structure C is as follows.

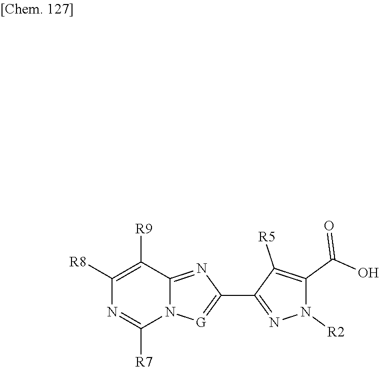

In Table 9, the structure D is as follows.

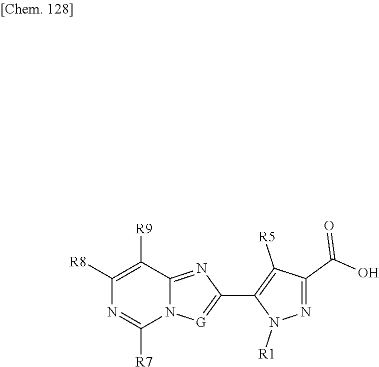

TABLE 9

| Compound No. | Structure | R1 | R2 | R5 | G | R7 | R8 | R9 |
|---|---|---|---|---|---|---|---|---|
| I-1 | C | — | Me | SEt | C—H | H | CF₃ | H |
| I-2 | C | — | Et | SEt | C—H | H | CF₃ | H |
| I-3 | C | — | Pr | SEt | C—H | H | CF₃ | H |
| I-4 | C | — | CH₂CF₃ | SEt | C—H | H | CF₃ | H |
| I-5 | C | — | 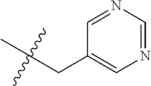 [SMILES: *Cc1cncnc1] | SEt | C—H | H | CF₃ | H |
| I-6 | D | CH₂CF₃ | — | SEt | C—H | H | CF₃ | H |
| I-7 | D | 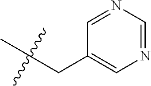 [SMILES: *Cc1cncnc1] | — | SEt | C—H | H | CF₃ | H |

Next, ¹H-NMR data of the synthesis intermediate compounds listed in Table 9 are shown in Table 10.

TABLE 10

| Compound | |
|---|---|
| No. | ¹H-NMR |
| I-1 | ¹H-NMR (CDCl₃) δ: 9.17 (1H, s), 8.53 (1H, s), 7.99 |
| | (1H, s), 4.37 (3H, s), 2.95 (2H, q, J = 7.4 Hz), 1.26 |
| | (3H, t, J = 7.4 Hz). |
| I-2 | ¹H-NMR (DMSO-D₆) δ: 13.95 (1H, br s), 9.66 (1H, d, |
| | J = 0.8 Hz), 8.88 (1H, s), 8.24 (1H, s), 4.51 (2H, q, |
| | J = 7.1 Hz), 2.82 (2H, q, J = 7.4 Hz), 1.41 (3H, t, |
| | J = 7.1 Hz), 1.08 (3H, t, J = 7.4 Hz). |
| I-3 | ¹H-NMR (DMSO-D₆) δ: 9.64 (1H, d, J = 0.6 Hz), |
| | 8.86 (1H, s), 8.22 (1H, s), 4.43 (2H, t, J = 7.0 Hz), |
| | 2.80 (2H, q, J = 7.3 Hz), 1.84-1.78 (2H, m), 1.06 |
| | (3H, t, J = 7.3 Hz), 0.84 (3H, t, J = 7.3 Hz). |
| I-4 | ¹H-NMR (CDCl₃) δ: 9.18 (1H, s), 8.59 (1H, s), 8.02 |
| | (1H, s), 5.53-5.46 (2H, m), 3.01 (2H, q, J = 7.4 Hz), |
| | 1.26 (3H, t, J = 7.4 Hz). |
| I-5 | ¹H-NMR (DMSO-D₆) δ: 9.64 (1H, d, J = 1.0 Hz), 9.13 |
| | (1H, s), 8.89 (1H, s), 8.71 (2H, s), 8.23 (1H, d, |
| | J = 0.7 Hz), 5.79 (2H, s), 2.82 (2H, q, J = 7.4 Hz), |
| | 1.06 (3H, t, J = 7.4 Hz). |
| I-6 | ¹H-NMR (DMSO-D₆) δ: 9.72 (1H, s), 9.01 (1H, s), |
| | 8.37 (1H, s), 6.91 (1H, br s), 5.83 (2H, q, J = 8.9 Hz), |
| | 2.83 (2H, q, J = 7.3 Hz), 1.16 (3H, t, J = 7.3 Hz). |
| I-7 | ¹H-NMR (CDCl₃) δ: 9.22 (1H, s), 9.14 (1H, s), 8.83 |
| | (1H, s), 8.79 (2H, s), 7.99 (1H, s), 6.17 (2H, s), |
| | 2.90 (2H, q, J = 7.4 Hz), 1.14 (3H, t, J = 7.4 Hz). |

Next, formulation examples containing the compound of the present invention as an active ingredient will be shown, but the present invention is not limited thereto. Note that, in the formulation examples, “part(s)” refers to “part(s) by weight”.

### Formulation Example 1

10 parts of the compound of the present invention represented by Formula (1-1) or Formula (1-2), 30 parts of polyoxyethylene aryl phenyl ether sulfate ammonium salt, and 160 parts of 1-butyl-2 pyrrolidone (NBP) were uniformly mixed to obtain an emulsion.

### Formulation Example 2

10 parts of the compound of the present invention represented by Formula (1-1) or Formula (1-2), 2 parts of sodium lauryl sulfate, 2 parts of dialkyl sulfosuccinate, 1 part of p-naphthalenesulfonic acid formalin condensate sodium salt, and 85 parts of diatomaceous earth were uniformly stirred and mixed to obtain a wettable powder.

### Formulation Example 3

0.3 parts of the compound of the present invention represented by Formula (1-1) or Formula (1-2) and 0.3 parts of white carbon were uniformly mixed, 99.2 parts of clay and 0.2 parts of DORILES A (manufactured by Sankyo Agro Co., Ltd.) were added, and uniformly pulverized and mixed to obtain a dustable powder.

### Formulation Example 4

3 parts of the compound of the present invention represented by Formula (1-1) or Formula (1-2), 1.5 parts of a polyoxyethylene/polyoxypropylene condensate, 3 parts of carboxymethylcellulose, 64.8 parts of clay, and 27.7 parts of talc were uniformly pulverized and mixed, water was added and kneaded, and then granulation and drying were performed, thereby obtaining granules.

### Formulation Example 5

10 parts of the compound of the present invention represented by Formula (1-1) or Formula (1-2), 3 parts of β-naphthalenesulfonic acid formalin condensate sodium salt, 1 part of tristyrylphenol, 5 parts of propylene glycol, 0.5 parts of a silicone-based antifoaming agent, and 33.5 parts of water were sufficiently stirred and mixed, and 0.3 parts of xanthan gum and 46.7 parts of water were mixed and stirred and mixed again, thereby obtaining a flowable.

### Formulation Example 6

20 parts of the compound of the present invention represented by Formula (1-1) or Formula (1-2), 6 parts of naphthalene sulfonic acid formaldehyde condensate metal salt, 1 part of dialkyl sulfosuccinic acid metal salt, and 73 parts of calcium carbonate were uniformly pulverized and mixed, and then water was added, kneaded, and granulated and dried, thereby obtaining water dispersible granules.

When using the formulation obtained above, the formulation is diluted 1 to 10,000 times with water to be sprayed, or directly sprayed without dilution.

Next, the usefulness of the compound of the present invention as a pest control agent will be specifically described in the following test examples, but the present invention is not limited thereto.

### Test Example 1

Insecticidal Test Against Nilaparvata lugens

2.5 ml of an acetone solution prepared to have a predetermined concentration of the test compound was sprayed on a rice (Oryza sativa) seedling, and the rice seedling was transferred to a glass tube in which water was retained. After air drying, 10 young larvae were released into the glass tube, and the glass tube was covered with a sponge stopper. The glass tube was allowed to stand in a thermostatic chamber at 25° C., and the numbers of living insects and dead insects were examined after 6 days.

As a result of the above test, Compound Nos. 1, 2, 3, 4, 7, 8, 9, 10, 11, 12, 13, 14,15,16,17, 18, 23, 24, 25, 27, 28, 29, 31, 32, 33, 34, 35, 36, 37, 38, 39, 40, 41, 42, 43, 44, 45, 47, 48, 49, 50, 51, 52, 53, 54, 55, 56, 57, 58, 59, 60, 61, 62, 63, 64, 65, 66, 67, 68, 70, 72, 73, 74, 75, 76, 77, 78, 79, 80, 81, 82, 83, 84, 85, 86, 87, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 131, 132, 134, 135, 136, 139, 141, 144, 145, 146, 147, 150, 151, 153, 154, 155, 156, 157, 158, 161, 162, 163, 169, 171, 172, 173, 175, 176, 177, 178, 180, 181, 182, 183, 184, 185, 186, 187, 188, 189, 192, 193, 194, 195, 200, 205, 206, 217, 219, 220, 221, 222, 223, 224, 225, 226, 227, 228, 229, 230, 232, 234, 235, 236, 237, 238, 240, 241, 242, 243, 245, 246, 247, 248, 249, 250, 251, 252, 253, 254, 255, 256, 257, 260, 261, 262, 263, 264, 265, 266, 267, 268, 269, 270, 271, 272, 273, 274, 275, 277, 278, 279, 281, 282, 283, 284, 285, 286, 287, 288, 289, 290, 291, 292, 293, 294, 295, 296, 300, 301, 302, 303, 305, 306, 307, 308, 309, 310, 311, 314, 315, 316, 319, 321, 322, 323, 324, 325, 326, 327, 328, 329, 330, 332, 335, 336, 337, 338, 339, 340, 341, 342, 343, 344, 345, 346, 347, 349, 350, 351, 352, 353, 354, 356, 358, 360, 361, 362, 363, 364, 366, 367, 368, 369, 370, 371, 372, 373, 374, 375, 376, 377, 378, 380, 381, 382, 383, 385, 387, 388, 389, 390, 391, 392, 393, 394, 395, 397, 398, 399, 400, 401, 402, 403, 404, 405, 406, 407, 408, 409, 410, 411, 412, 413, 414, 415, 416, 417, 419, 420, 421, 422, 423, 424, 425, 426, 428, 429, 430, 431, 433, 434, 435, and 436 showed a mortality rate of 50% or more at a concentration of 100 ppm.

### Test Example 2

Insecticidal Test Against Nilaparvata lugens

12.5 ml of a 2% acetone aqueous solution in which the test compound was adjusted to a predetermined concentration was placed in a container, and a root part of a rice (Oryza sativa) seedling fixed with a sponge and a plastic cylinder was immersed. 10 young larvae were released into the container, and the container was covered with a plastic lid. The container was allowed to stand in a thermostatic chamber at 25° C., and the numbers of living insects and dead insects were examined after 6 days.

As a result of the above test, Compound Nos. 1, 2, 3, 8, 9, 11, 12, 14, 15, 16, 17, 18, 20, 25, 30, 31, 32, 33, 37, 38, 39, 40, 41, 42, 43, 47, 48, 50, 51, 52, 53, 54, 56, 59, 60, 61, 62, 63, 64, 65, 67, 70, 73, 74, 76, 77, 78, 79, 80, 81, 82, 89, 90, 94, 97, 100, 101, 103, 108, 119, 120, 126, 132, 134, 135, 136, 145, 146, 147, 151, 153, 159, 164, 169, 172, 173, 176, 181, 183, 185, 187, 188, 199, 208, 218, 222, 223, 227, 228, 232, 236, 237, 238, 240, 243, 251, 255, 256, 275, 279, 281, 285, 286, 287, 289, 290, 292, 293, 294, 296, 322, 327, 328, 330, 334, 338, 341, 351, 362, 367, 370, 373, 383, 388, 390, 391, 392, 394, 399, 402, 403, 405, 406, 407, 410, 411,413,414,415,416,417, 419, 421, 422, 423, 424, 425, 426, 428, 431, 433, and 436 showed a mortality rate of 50% or more at a concentration of 100 ppm.

### Test Example 3

Insecticidal Test Against Myzus persicae

2.5 ml of a 20% acetone aqueous solution prepared to have a predetermined concentration of the test compound was sprayed on the leaves of radish (Raphanus sativus) in which the roots retained water and 25 young larvae were parasitized. After air drying, it was allowed to stand in a thermostatic chamber at 25° C., and the number of living insects and the number of dead insects were examined after 5 days.

As a result of the above test, Compound Nos. 1, 2, 3, 5, 6, 8, 9, 11, 14, 15, 16, 17, 18, 23, 25, 27, 31, 32, 34, 36, 38, 39, 40, 42, 43, 44, 45, 48, 52, 53, 54, 56, 58, 59, 60, 61, 62, 63, 64, 65, 66, 67, 68, 69, 70, 73, 74, 75, 86, 90, 97, 98, 99, 100, 102, 104, 107, 109, 110, 111, 112, 113, 115, 116, 117, 118, 119, 120, 121, 122, 123, 124, 125, 126, 127, 131, 132, 133, 136, 139, 144, 146, 148, 150, 151, 153, 160, 162, 167, 169, 171, 172, 173, 175, 176, 178, 181, 182, 183, 185, 187, 188, 189, 193, 204, 205, 206, 209, 212, 217, 218, 219, 220, 221, 222, 223, 225, 226, 227, 228, 229, 230, 232, 233, 234, 236, 237, 238, 240, 241, 243, 245, 246, 247, 248, 249, 251, 253, 254, 255, 256, 257, 260, 261, 263, 264, 265, 266, 267, 269, 271, 272, 273, 274, 275, 277, 281, 283, 285, 286, 289, 290, 291, 292, 293, 294, 296, 300, 301, 302, 305, 309, 310, 311, 316, 322, 324, 326, 327, 328, 329, 330, 332, 334, 336, 341, 342, 343, 344, 351, 360, 361, 363, 368, 370, 371, 373, 375, 376, 379, 382, 383, 387, 389, 390, 391, 394, 395, 397, 399,400,403,404,405,406,407,408,409,410,411,413,414,415,416,417,419, 420, 421, 422, 424, 426, 431, 433, and 436 showed a mortality rate of 50% or more at a concentration of 100 ppm.

### Test Example 4

Insecticidal Test Against Myzus persicae

12.5 ml of a 2% acetone aqueous solution in which the test compound was adjusted to a predetermined concentration was placed in a container, and a root part of a young radish (Raphanus sativus) seedling that was fixed with a sponge and parasitized with 25 young larvae was immersed. The container was allowed to stand in a thermostatic chamber at 25° C., and the numbers of living insects and dead insects were examined after 5 days.

As a result of the above test, Compound Nos. 1, 2, 3, 8, 9, 56, 58, 59, and 64 showed a mortality rate of 50% or more at a concentration of 100 ppm.

### Test Example 5

Insecticidal Test Against Spodopteta litura

Cabbage (Brassicae oleracea) leaf pieces were immersed in a chemical solution prepared to have a predetermined concentration of the test compound for 30 seconds, air-dried, then placed in a 7 cm polyethylene cup with a filter paper laid thereon, and five 2nd instar larvae of Spodoptera litura were released. The container was allowed to stand in a thermostatic chamber at 25° C., and the numbers of living insects and dead insects were examined after 6 days.

As a result of the above test, Compound Nos. 65, 67, 70, 77, 84, 86, 122, 129, 137, 144, 161, 162, 174, 179, 180, 181, 190, 191, 196, 198,201,202,203,210,211, 213, 214, 216, 217, 227, 232, 255, 258, 263, 266, 275, 292, 293, 328, 333, 343, 353, 355, 356, 358, 359, 364, 383, 407, 410, 413, 416, 420, 422, and 427 showed a mortality rate of 50% or more at a concentration of 1,000 ppm.

### Test Example 6

Insecticidal Test Against Sogatella furcifera

2.5 ml of an acetone solution prepared to have a predetermined concentration of the test compound was sprayed on a rice (Oryza sativa) seedling, and the rice seedling was transferred to a glass tube in which water was retained. After air drying, 10 young larvae were released into the glass tube, and the glass tube was covered with a sponge stopper. The glass tube was allowed to stand in a thermostatic chamber at 25° C., and the numbers of living insects and dead insects were examined after 6 days.

As a result of the above test, Compound Nos. 1, 3, 8, 9, 11, 12, 15, 16, 17, 18, 19, 20, 21, 22, 23, 24, 25, 26, 27, 29, 30, 31, 32, 33, 34, 35, 36, 37, 38, 39, 40, 41, 42, 43, 44, 46, 47, 48, 49, 50, 52, 53, 54, 55, 56, 57, 58, 59, 60, 61, 62, 63, 64, 65, 66, 67, 68, 69, 71, 73, 74, 75, 76, 77, 78, 79, 80, 81, 82, 88, 89, 90, 92, 93, 94, 95, 96, 97, 98, 99, 100, 101, 103, 107, 108, 109, 110, 111, 112, 113, 114, 115, 116, 117, 118, 119, 120, 121, 122, 123, 124, 125, 126, 127, 128, 130, 131, 132, 134, 135, 136, 138, 139, 140, 141, 142, 143, 144, 145, 146, 147, 149, 151, 152, 153, 165, 168, 169, 170, 171, 172, 173, 175, 176, 177, 178, 179, 180, 181, 182, 183, 184, 185, 186, 187, 189, 193, 194, 205, 207, 213, 215, 217, 219, 220, 222, 223, 225, 226, 227, 228, 229, 230, 231, 232, 234, 235, 236, 237, 238, 239, 240, 241, 242, 243, 244, 245, 246, 247, 248, 249, 250, 251, 252, 253, 254, 255, 256, 257, 259, 260, 261, 262, 263, 264, 265, 266, 267, 268, 269, 270, 271, 273, 274, 275, 276, 277, 278, 280, 281, 282, 283, 284, 286, 288, 290, 291, 292, 293, 294, 295, 296, 298, 299, 300, 301, 302, 305, 306, 307, 309, 310, 313, 316, 317, 319, 320, 321, 322, 324, 325, 326, 327, 328, 329, 330, 336, 337, 338, 339, 340, 341, 342, 343, 344, 346, 348, 351, 352, 353, 356, 358, 360, 361, 363, 364, 365, 367, 368, 369, 370, 371, 372, 373, 374, 375, 376, 381, 382, 383, 385, 386, 387, 388, 389, 390, 391, 392, 393, 394, 395, 396, 397, 399, 401, 403, 404, 405, 406, 407, 408, 409,410, 411, 412, 413, 414, 415, 416, 417, 418, 419, 420, 421, 422, 423, 424, 425, 426, 428, 431, 432, 433, and 436 showed a mortality rate of 50% or more at 100 ppm.

### INDUSTRIAL APPLICABILITY

Since the pyrazole compound of the present invention is a novel compound and can control pests, particularly arthropod pests, the pyrazole compound has utility value as an agricultural chemical, for example, an agricultural or horticultural pest control agent, particularly an agricultural or horticultural insecticide.

All documents, patent applications, and technical standards described in the present specification are incorporated herein by reference to the same extent as if each individual document, patent application, and technical standard were specifically and individually indicated to be incorporated by reference.

1. A compound represented by Formula (1-1) or Formula (1-2), 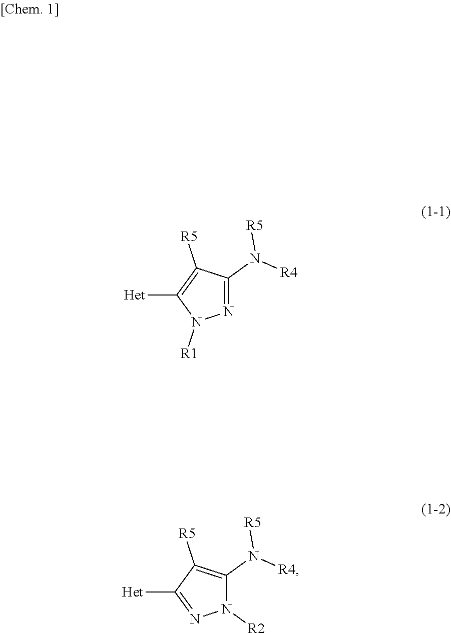
or a salt thereof
[wherein, R1 represents
a hydrogen atom,
a C1 to C6 alkyl group optionally substituted with a substituent A,
a C1 to C6 haloalkyl group,
a C3 to C8 cycloalkyl group optionally substituted with a substituent B,
a C2 to C6 alkenyl group optionally substituted with a substituent A,
a C2 to C6 haloalkenyl group,
a C2 to C6 alkynyl group optionally substituted with a substituent A,
a C2 to C6 haloalkynyl group,
a phenyl group optionally substituted with 0 to 5 substituents B,
a 5- or 6-membered aromatic heterocyclic group optionally substituted with 0 to 4 substituents B,
Rx1Rx2NC(═O)— (where Rx1 and Rx2 each independently represent a hydrogen atom, a hydroxyl group, a cyano group, a C1 to C6 alkyl group optionally substituted with a substituent A, a C1 to C6 haloalkyl group, a C3 to C8 cycloalkyl group optionally substituted with a substituent B, a C2 to C6 alkenyl group optionally substituted with a substituent A, a C2 to C6 haloalkenyl group, a C2 to C6 alkynyl group optionally substituted with a substituent A, a C2 to C6 haloalkynyl group, a C1 to C6 alkoxy group, a C1 to C6 haloalkoxy group, a C3 to C8 cycloalkoxy group, a phenyl group optionally substituted with 0 to 5 substituents B, or a 5- or 6-membered aromatic heterocyclic group optionally substituted with 0 to 4 substituents B, or Rx1 and Rx2 together with a nitrogen atom to which they are bonded form an aziridinyl group, an azetidinyl group, a pyrrolidinyl group, a piperidinyl group, a piperazinyl group, a morpholinyl group, a thiomorpholinyl group, a dioxothiomorpholinyl group, a thiazolidinyl group, an azepanyl group, or an azocanyl group),
Rx3C(═O)— (where Rx3 represents a hydrogen atom, a hydroxyl group, a halogen atom, a C1 to C6 alkyl group optionally substituted with a substituent A, a C1 to C6 haloalkyl group, a C3 to C8 cycloalkyl group optionally substituted with a substituent B, a C2 to C6 alkenyl group optionally substituted with a substituent A, a C2 to C6 haloalkenyl group, a C2 to C6 alkynyl group optionally substituted with a substituent A, a C2 to C6 haloalkynyl group, a phenyl group optionally substituted with 0 to 5 substituents B, or a 5- or 6-membered aromatic heterocyclic group optionally substituted with 0 to 4 substituents B),
Rx3OC(═O)— (where Rx3 has the same meaning as above),
Rx4S(O)p- (where Rx4 represents a hydroxyl group, a halogen atom, a C1 to C6 alkyl group optionally substituted with a substituent A, a C1 to C6 haloalkyl group, a C3 to C8 cycloalkyl group optionally substituted with a substituent B, a C2 to C6 alkenyl group optionally substituted with a substituent A, a C2 to C6 haloalkenyl group, a C2 to C6 alkynyl group optionally substituted with a substituent A, a C2 to C6 haloalkynyl group, or a phenyl group optionally substituted with 0 to 5 substituents B, and p represents an integer of 0, 1, or 2), or
a 3- to 6-membered ring group containing one or two oxygen atoms;
R2 represents
a hydrogen atom,
a C1 to C6 alkyl group optionally substituted with a substituent A,
a C1 to C6 haloalkyl group,
a C3 to C8 cycloalkyl group optionally substituted with a substituent B,
a C2 to C6 alkenyl group optionally substituted with a substituent A,
a C2 to C6 haloalkenyl group,
a C2 to C6 alkynyl group optionally substituted with a substituent A,
a C2 to C6 haloalkynyl group,
a phenyl group optionally substituted with 0 to 5 substituents B,
a 5- or 6-membered aromatic heterocyclic group optionally substituted with 0 to 4 substituents B,
Rx1Rx2NC(═O)— (where, Rx1 and Rx2 have the same meanings as above),
Rx3C(═O)— (where, Rx3 has the same meaning as above),
Rx3OC(═O)— (where, Rx3 has the same meaning as above),
Rx4S(O)p- (where, Rx4 and p have the same meanings as above), or
a 3- to 6-membered ring group containing one or two oxygen atoms;
R3 represents
a hydrogen atom,
a C1 to C6 alkyl group optionally substituted with a substituent A,
a C1 to C6 haloalkyl group,
a C3 to C8 cycloalkyl group optionally substituted with a substituent B,
a C2 to C6 alkenyl group optionally substituted with a substituent A,
a C2 to C6 haloalkenyl group,
a C2 to C6 alkynyl group optionally substituted with a substituent A, or
a C2 to C6 haloalkynyl group,
Rx1Rx2NC(═O)— (where, Rx1 and Rx2 have the same meanings as above),
Rx3C(═O)— (where, Rx3 has the same meaning as above),
Rx3OC(═O)— (where, Rx3 has the same meaning as above), or
Rx4S(O)p- (where, Rx4 and p have the same meanings as above);
R4 represents
a hydrogen atom,
a C1 to C6 alkyl group optionally substituted with a substituent A,
a C1 to C6 haloalkyl group,
a C3 to C8 cycloalkyl group optionally substituted with a substituent B,
a C2 to C6 alkenyl group optionally substituted with a substituent A,
a C2 to C6 haloalkenyl group,
a C2 to C6 alkynyl group optionally substituted with a substituent A,
a C2 to C6 haloalkynyl group,
Rx1Rx2NC(═O)— (where, Rx1 and Rx2 have the same meanings as above),
Rx3C(═O)— (where, Rx3 has the same meaning as above),
Rx3OC(═O)— (where, Rx3 has the same meaning as above), or
Rx4S(O)p- (where, Rx4 and p have the same meanings as above);
R5 represents
a cyano group,
a halogen atom,
a C1 to C6 alkoxy group optionally substituted with a substituent A,
a C1 to C6 haloalkoxy group,
a C3 to C8 cycloalkoxy group optionally substituted with a substituent A,
a C2 to C6 alkenyl group optionally substituted with a substituent A,
a C2 to C6 haloalkenyl group,
a C2 to C6 alkynyl group optionally substituted with a substituent A,
a C2 to C6 haloalkynyl group, or
Rx4S(O)p- (where, Rx4 and p have the same meanings as above);
Het represents Formula (Het); 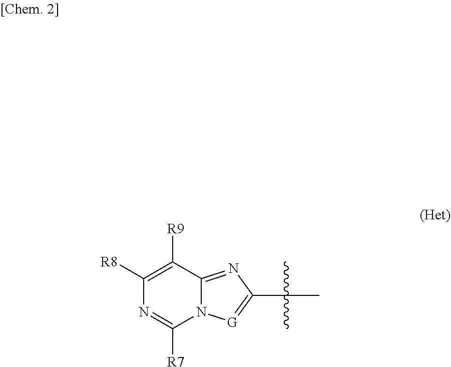
G represents C—R6 or a nitrogen atom;
R6 represents
a hydrogen atom,
a cyano group,
a halogen atom,
a C1 to C6 alkyl group optionally substituted with a substituent A,
a C1 to C6 haloalkyl group,
a C3 to C8 cycloalkyl group optionally substituted with a substituent B,
a C2 to C6 alkenyl group optionally substituted with a substituent A,
a C2 to C6 haloalkenyl group,
a C2 to C6 alkynyl group optionally substituted with a substituent A, or
a C2 to C6 haloalkynyl group;
R7 represents
a hydrogen atom,
a hydroxyl group,
a cyano group,
a halogen atom,
a C1 to C6 alkyl group optionally substituted with a substituent A,
a C1 to C6 haloalkyl group,
a C3 to C8 cycloalkyl group optionally substituted with a substituent B,
a C2 to C6 alkenyl group optionally substituted with a substituent A,
a C2 to C6 haloalkenyl group,
a C2 to C6 alkynyl group optionally substituted with a substituent A,
a C2 to C6 haloalkynyl group,
a C1 to C6 alkoxy group optionally substituted with a substituent A,
a C1 to C6 haloalkoxy group,
a C3 to C8 cycloalkoxy group optionally substituted with a substituent A, or
Rx4S(O)p- (where, Rx4 and p have the same meanings as above);
R8 represents
a hydrogen atom,
a hydroxyl group,
a cyano group,
a halogen atom,
a C1 to C6 alkyl group optionally substituted with a substituent A,
a C1 to C6 haloalkyl group,
a C3 to C8 cycloalkyl group optionally substituted with a substituent B,
a C1 to C6 alkoxy group optionally substituted with a substituent A,
a C1 to C6 haloalkoxy group,
a C3 to C8 cycloalkoxy group optionally substituted with a substituent A, or
Rx4S(O)p- (where, Rx4 and p have the same meanings as above);
R9 represents
a hydrogen atom,
a hydroxyl group,
a cyano group,
a halogen atom,
a C1 to C6 alkyl group optionally substituted with a substituent A,
a C1 to C6 haloalkyl group,
a C3 to C8 cycloalkyl group optionally substituted with a substituent B,
a C1 to C6 alkoxy group optionally substituted with a substituent A,
a C1 to C6 haloalkoxy group,
a C3 to C8 cycloalkoxy group optionally substituted with a substituent A, or
Rx4S(O)p- (where, Rx4 and p have the same meanings as above);
the substituent A is at least one selected from the group consisting of
a hydroxyl group,
a cyano group,
a nitro group,
a halogen atom,
a C3 to C8 cycloalkyl group,
a C1 to C6 alkoxy group,
a C1 to C6 haloalkoxy group,
a C3 to C8 cycloalkoxy group,
a phenyl group optionally substituted with 0 to 5 substituents C,
a 5- or 6-membered aromatic heterocyclic group optionally substituted with 0 to 4 substituents C, and
a 3- to 6-membered ring group containing one or two oxygen atoms;
the substituent B is at least one selected from the group consisting of
a hydroxyl group,
a cyano group,
a nitro group,
a halogen atom,
a C1 to C6 alkyl group,
a C1 to C6 haloalkyl group,
a C3 to C8 cycloalkyl group,
a C1 to C6 alkoxy group,
a C1 to C6 haloalkoxy group,
a C3 to C8 cycloalkoxy group, and
Rx5S(O)p- (where Rx5 represents a hydroxyl group, a halogen atom, a C1 to C6 alkyl group, a C1 to C6 haloalkyl group, a C3 to C8 cycloalkyl group, a C2 to C6 alkenyl group, a C2 to C6 haloalkenyl group, a C2 to C6 alkynyl group, or a C2 to C6 haloalkynyl group, and p has the same meaning as above); and
the substituent C is at least one independently selected from the group consisting of
a cyano group,
a halogen atom,
a C1 to C6 alkyl group,
a C1 to C6 haloalkyl group,
a C3 to C8 cycloalkyl group,
a C1 to C6 alkoxy group,
a C1 to C6 haloalkoxy group,
a C3 to C8 cycloalkoxy group, and
Rx5S(O)p- (where, Rx5 and p have the same meanings as above)].

2. The compound or the salt thereof according to claim 1, wherein R1 represents
a hydrogen atom,
a C1 to C6 alkyl group optionally substituted with a substituent A,
a C1 to C6 haloalkyl group,
a C3 to C8 cycloalkyl group optionally substituted with a substituent B,
a C2 to C6 alkenyl group optionally substituted with a substituent A,
a C2 to C6 alkynyl group optionally substituted with a substituent A, or
Rx1Rx2NC(═O)— (where Rx1 and Rx2 each independently represent a hydrogen atom, a hydroxyl group, a cyano group, a C1 to C6 alkyl group optionally substituted with a substituent A, a C1 to C6 haloalkyl group, a C3 to C8 cycloalkyl group optionally substituted with a substituent B, a C2 to C6 alkenyl group optionally substituted with a substituent A, a C2 to C6 haloalkenyl group, a C2 to C6 alkynyl group optionally substituted with a substituent A, a C2 to C6 haloalkynyl group, a C1 to C6 alkoxy group, a C1 to C6 haloalkoxy group, a C3 to C8 cycloalkoxy group, a phenyl group optionally substituted with 0 to 5 substituents B, or a 5- or 6-membered aromatic heterocyclic group optionally substituted with 0 to 4 substituents B, or Rx1 and Rx2 together with a nitrogen atom to which they are bonded form an aziridinyl group, an azetidinyl group, a pyrrolidinyl group, a piperidinyl group, a piperazinyl group, a morpholinyl group, a thiomorpholinyl group, a dioxothiomorpholinyl group, a thiazolidinyl group, an azepanyl group, or an azocanyl group);
R2 represents
a hydrogen atom,
a C1 to C6 alkyl group optionally substituted with a substituent A,
a C1 to C6 haloalkyl group,
a C3 to C8 cycloalkyl group optionally substituted with a substituent B,
a C2 to C6 alkenyl group optionally substituted with a substituent A,
a C2 to C6 alkynyl group optionally substituted with a substituent A, or
Rx1Rx2NC(═O)— (where Rx1 and Rx2 have the same meanings as above);
R3 represents
a hydrogen atom,
a C1 to C6 alkyl group optionally substituted with a substituent A,
a C1 to C6 haloalkyl group,
a C3 to C8 cycloalkyl group optionally substituted with a substituent B,
a C2 to C6 alkenyl group optionally substituted with a substituent A,
a C2 to C6 alkynyl group optionally substituted with a substituent A,
Rx1Rx2NC(═O)— (where Rx1 and Rx2 have the same meanings as above),
Rx3C(═O)— (where Rx3 represents a hydrogen atom, a hydroxyl group, a halogen atom, a C1 to C6 alkyl group optionally substituted with a substituent A, a C1 to C6 haloalkyl group, a C3 to C8 cycloalkyl group optionally substituted with a substituent B, a C2 to C6 alkenyl group optionally substituted with a substituent A, a C2 to C6 haloalkenyl group, a C2 to C6 alkynyl group optionally substituted with a substituent A, a C2 to C6 haloalkynyl group, a phenyl group optionally substituted with 0 to 5 substituents B, or a 5- or 6-membered aromatic heterocyclic group optionally substituted with 0 to 4 substituents B), or
Rx3OC(═O)— (where Rx3 has the same meaning as above);
R4 represents
a hydrogen atom,
a C1 to C6 alkyl group optionally substituted with a substituent A,
a C1 to C6 haloalkyl group,
a C3 to C8 cycloalkyl group optionally substituted with a substituent B,
a C2 to C6 alkenyl group optionally substituted with a substituent A,
a C2 to C6 alkynyl group optionally substituted with a substituent A,
Rx1Rx2NC(═O)— (where Rx1 and Rx2 have the same meanings as above),
Rx3C(═O)— (where Rx3 has the same meaning as above),
Rx3OC(═O)— (where Rx3 has the same meaning as above), or
Rx4S(O)p- (where Rx4 represents a hydroxyl group, a halogen atom, a C1 to C6 alkyl group optionally substituted with a substituent A, a C1 to C6 haloalkyl group, a C3 to C8 cycloalkyl group optionally substituted with a substituent B, a C2 to C6 alkenyl group optionally substituted with a substituent A, a C2 to C6 haloalkenyl group, a C2 to C6 alkynyl group optionally substituted with a substituent A, a C2 to C6 haloalkynyl group, or a phenyl group optionally substituted with 0 to 5 substituents B, and p represents an integer of 0, 1, or 2);
R5 represents
a cyano group,
a halogen atom,
a C1 to C6 alkoxy group optionally substituted with a substituent A,
a C1 to C6 haloalkoxy group,
a C3 to C8 cycloalkoxy group optionally substituted with a substituent A, or
Rx4S(O)p- (where Rx4 and p have the same meanings as above);
R6 represents
a hydrogen atom,
a cyano group,
a halogen atom,
a C1 to C6 alkyl group optionally substituted with a substituent A,
a C1 to C6 haloalkyl group, or
a C3 to C8 cycloalkyl group optionally substituted with a substituent B;
R7 represents
a hydrogen atom,
a hydroxyl group,
a cyano group,
a halogen atom,
a C1 to C6 alkyl group optionally substituted with a substituent A,
a C1 to C6 haloalkyl group,
a C3 to C8 cycloalkyl group optionally substituted with a substituent B,
a C1 to C6 alkoxy group optionally substituted with a substituent A,
a C1 to C6 haloalkoxy group,
a C3 to C8 cycloalkoxy group optionally substituted with a substituent A, or
Rx4S(O)p- (where Rx4 and p have the same meanings as above);
R8 represents
a hydrogen atom,
a halogen atom,
a C1 to C6 alkyl group optionally substituted with a substituent A,
a C1 to C6 haloalkyl group,
a C3 to C8 cycloalkyl group optionally substituted with a substituent B,
a C1 to C6 haloalkoxy group, or
Rx4S(O)p- (where Rx4 and p have the same meanings as above); and
R9 represents
a hydrogen atom,
a halogen atom,
a C1 to C6 alkyl group optionally substituted with a substituent A,
a C1 to C6 haloalkyl group, or
Rx4S(O)p- (where Rx4 and p have the same meanings as above).

3. The compound or the salt thereof according to claim 2, wherein R1 represents
a hydrogen atom,
a C1 to C6 alkyl group optionally substituted with a substituent A, or
a C1 to C6 haloalkyl group;
R2 represents
a hydrogen atom,
a C1 to C6 alkyl group optionally substituted with a substituent A,
a C1 to C6 haloalkyl group,
a C3 to C8 cycloalkyl group optionally substituted with a substituent B, or
Rx1Rx2NC(═O)— (where Rx1 and Rx2 each independently represent a hydrogen atom, a hydroxyl group, a cyano group, a C1 to C6 alkyl group optionally substituted with a substituent A, a C1 to C6 haloalkyl group, a C3 to C8 cycloalkyl group optionally substituted with a substituent B, a C2 to C6 alkenyl group optionally substituted with a substituent A, a C2 to C6 haloalkenyl group, a C2 to C6 alkynyl group optionally substituted with a substituent A, a C2 to C6 haloalkynyl group, a C1 to C6 alkoxy group, a C1 to C6 haloalkoxy group, a C3 to C8 cycloalkoxy group, a phenyl group optionally substituted with 0 to 5 substituents B, or a 5- or 6-membered aromatic heterocyclic group optionally substituted with 0 to 4 substituents B, or Rx1 and Rx2 together with a nitrogen atom to which they are bonded form an aziridinyl group, an azetidinyl group, a pyrrolidinyl group, a piperidinyl group, a piperazinyl group, a morpholinyl group, a thiomorpholinyl group, a dioxothiomorpholinyl group, a thiazolidinyl group, an azepanyl group, or an azocanyl group);
R3 represents
a hydrogen atom,
a C1 to C6 alkyl group optionally substituted with a substituent A,
a C1 to C6 haloalkyl group,
a C3 to C8 cycloalkyl group optionally substituted with a substituent B,
Rx1Rx2NC(═O)— (where Rx1 and Rx2 have the same meanings as above),
Rx3C(═O)— (where Rx3 represents a hydrogen atom, a hydroxyl group, a halogen atom, a C1 to C6 alkyl group optionally substituted with a substituent A, a C1 to C6 haloalkyl group, a C3 to C8 cycloalkyl group optionally substituted with a substituent B, a C2 to C6 alkenyl group optionally substituted with a substituent A, a C2 to C6 haloalkenyl group, a C2 to C6 alkynyl group optionally substituted with a substituent A, a C2 to C6 haloalkynyl group, a phenyl group optionally substituted with 0 to 5 substituents B, or a 5- or 6-membered aromatic heterocyclic group optionally substituted with 0 to 4 substituents B), or
Rx3OC(═O)— (where Rx3 has the same meaning as above);
R4 represents
a hydrogen atom,
a C1 to C6 alkyl group optionally substituted with a substituent A,
a C1 to C6 haloalkyl group,
a C3 to C8 cycloalkyl group optionally substituted with a substituent B,
Rx1Rx2NC(═O)— (where Rx1 and Rx2 have the same meanings as above),
Rx3C(═O)— (where Rx3 has the same meaning as above),
Rx3OC(═O)— (where Rx3 has the same meaning as above), or
Rx4S(O)p-(where Rx4 represents a hydroxyl group, a halogen atom, a C1 to C6 alkyl group optionally substituted with a substituent A, a C1 to C6 haloalkyl group, a C3 to C8 cycloalkyl group optionally substituted with a substituent B, a C2 to C6 alkenyl group optionally substituted with a substituent A, a C2 to C6 haloalkenyl group, a C2 to C6 alkynyl group optionally substituted with a substituent A, a C2 to C6 haloalkynyl group, or a phenyl group optionally substituted with 0 to 5 substituents B, and p represents an integer of 0, 1, or 2);
R5 represents
a cyano group,
a halogen atom,
a C1 to C6 alkoxy group optionally substituted with a substituent A, or
Rx4S(O)p- (where Rx4 and p have the same meanings as above);
R6 represents
a hydrogen atom,
a halogen atom,
a C1 to C6 alkyl group optionally substituted with a substituent A, or
a C1 to C6 haloalkyl group;
R7 represents
a hydrogen atom,
a hydroxyl group,
a cyano group,
a halogen atom,
a C1 to C6 alkyl group optionally substituted with a substituent A,
a C1 to C6 haloalkyl group,
a C3 to C8 cycloalkyl group optionally substituted with a substituent B,
a C1 to C6 alkoxy group optionally substituted with a substituent A,
a C1 to C6 haloalkoxy group, or
Rx4S(O)p- (where Rx4 and p have the same meanings as above);
R8 represents
a halogen atom,
a C1 to C6 haloalkyl group,
a C1 to C6 haloalkoxy group, or
Rx4S(O)p- (where Rx4 and p have the same meanings as above); and
R9 represents
a hydrogen atom,
a halogen atom, or
a C1 to C6 haloalkyl group.

4. The compound or the salt thereof according to claim 3, wherein R1 represents
a C1 to C6 alkyl group optionally substituted with a substituent A, or
a C1 to C6 haloalkyl group;
R2 represents
a C1 to C6 alkyl group optionally substituted with a substituent A,
a C1 to C6 haloalkyl group, or
a C3 to C8 cycloalkyl group optionally substituted with a substituent B;
R3 represents
a hydrogen atom,
a C1 to C6 alkyl group optionally substituted with a substituent A,
a C1 to C6 haloalkyl group, or
Rx3C(═O)— (where Rx3 represents a hydrogen atom, a hydroxyl group, a halogen atom, a C1 to C6 alkyl group optionally substituted with a substituent A, a C1 to C6 haloalkyl group, a C3 to C8 cycloalkyl group optionally substituted with a substituent B, a C2 to C6 alkenyl group optionally substituted with a substituent A, a C2 to C6 haloalkenyl group, a C2 to C6 alkynyl group optionally substituted with a substituent A, a C2 to C6 haloalkynyl group, a phenyl group optionally substituted with 0 to 5 substituents B, or a 5- or 6-membered aromatic heterocyclic group optionally substituted with 0 to 4 substituents B);
R4 represents
a hydrogen atom,
a C1 to C6 alkyl group optionally substituted with a substituent A,
Rx1Rx2NC(═O)— (where Rx1 and Rx2 each independently represent a hydrogen atom, a hydroxyl group, a cyano group, a C1 to C6 alkyl group optionally substituted with a substituent A, a C1 to C6 haloalkyl group, a C3 to C8 cycloalkyl group optionally substituted with a substituent B, a C2 to C6 alkenyl group optionally substituted with a substituent A, a C2 to C6 haloalkenyl group, a C2 to C6 alkynyl group optionally substituted with a substituent A, a C2 to C6 haloalkynyl group, a C1 to C6 alkoxy group, a C1 to C6 haloalkoxy group, a C3 to C8 cycloalkoxy group, a phenyl group optionally substituted with 0 to 5 substituents B, or a 5- or 6-membered aromatic heterocyclic group optionally substituted with 0 to 4 substituents B, or Rx1 and Rx2 together with a nitrogen atom to which they are bonded form an aziridinyl group, an azetidinyl group, a pyrrolidinyl group, a piperidinyl group, a piperazinyl group, a morpholinyl group, a thiomorpholinyl group, a dioxothiomorpholinyl group, a thiazolidinyl group, an azepanyl group, or an azocanyl group),
Rx3C(═O)— (where Rx3 has the same meaning as above),
Rx3OC(═O)— (where Rx3 has the same meaning as above), or
Rx4S(O)p- (where Rx4 represents a hydroxyl group, a halogen atom, a C1 to C6 alkyl group optionally substituted with a substituent A, a C1 to C6 haloalkyl group, a C3 to C8 cycloalkyl group optionally substituted with a substituent B, a C2 to C6 alkenyl group optionally substituted with a substituent A, a C2 to C6 haloalkenyl group, a C2 to C6 alkynyl group optionally substituted with a substituent A, a C2 to C6 haloalkynyl group, or a phenyl group optionally substituted with 0 to 5 substituents B, and p represents an integer of 0, 1, or 2);
R5 represents
Rx4S(O)p- (where Rx4 and p have the same meanings as above);
R6 represents
a hydrogen atom,
a halogen atom, or
a C1 to C6 alkyl group optionally substituted with a substituent A;
R7 represents
a hydrogen atom,
a hydroxyl group,
a halogen atom,
a C1 to C6 alkyl group optionally substituted with a substituent A,
a C3 to C8 cycloalkyl group optionally substituted with a substituent B,
a C1 to C6 alkoxy group optionally substituted with a substituent A,
a C1 to C6 haloalkoxy group, or
Rx4S(O)p- (where Rx4 and p have the same meanings as above);
R8 represents
a C1 to C6 haloalkyl group; and
R9 represents
a hydrogen atom.

5. A pest control agent comprising the compound or the salt thereof according to claim 1 as an active ingredient.

6. The pest control agent according to claim 5, wherein the pest is an arthropod pest.

7. A method for controlling a pest, comprising applying the pest control agent according to claim 5 to a plant, a seed of a plant, or soil for cultivating a plant.

8. The method for controlling a pest according to claim 7, wherein the pest is an arthropod pest.
